(19) United States
(12) Patent Application Publication
(10) Pub. No.: US 2023/0340469 A1
(43) Pub. Date: Oct. 26, 2023

(54) TRANS-SPLICING SYSTEM FOR TISSUE-SPECIFIC REPLACEMENT OF RNA SEQUENCES

(71) Applicant: Tacit Therapeutics, Inc., South San Francisco, CA (US)

(72) Inventor: David A. Nelles, San Francisco, CA (US)

(21) Appl. No.: 18/189,896

(22) Filed: Mar. 24, 2023

Related U.S. Application Data
(63) Continuation of application No. PCT/US2021/052315, filed on Sep. 28, 2021.
(60) Provisional application No. 63/084,027, filed on Sep. 28, 2020.

(51) Int. Cl. C12N 15/11; C12N 15/86

(52) U.S. Cl. CPC C12N 15/111; C12N 15/86; C12N 2750/14143; C12N 2310/124; C12N 2320/33

(57) ABSTRACT

Disclosed are compositions comprising: a trans-splicing nucleic acid comprising (a) one or more replacement domains that encode a therapeutic sequence; and (b) one or more intronic domains that promote RNA splicing of the replacement domain primarily in a specific tissue or cell type; and (c) one or more antisense domains that promote binding to a target RNA molecule, wherein an RNA trans-splicing reaction promotes insertion of the replacement domain into a target RNA. Methods of making and methods of using compositions of the disclosure are also provided, including but not limited to compositions of the disclosure that may be used in the treatment of a disease or disorder in a patient or subject. Example disease or disorders of the disclosure include genetic and epigenetic diseases or disorders.

### CROSS-REFERENCE

This application is a continuation of International Application No. PCT/US2021/052315, filed on Sep. 28, 2021, which claims priority to U.S. provisional application No. 63/084,027, filed on Sep. 28, 2020, the entirety of which are hereby incorporated by reference herein.

### SEQUENCE LISTING

The instant application contains a Sequence Listing which has been submitted electronically in XML format is hereby incorporated by reference in its entirety. Said XML copy, created on Jul. 18, 2023, is named 63827_702_301_SL.xml and is 1,655,569 bytes in size.

### FIELD OF THE DISCLOSURE

The is a disclosure directed to gene therapy, molecular biology, and compositions and methods for altering the sequence composition of RNA molecules.

### BACKGROUND

There is a long-felt but unmet need in the art for, 1) controlling the location and timing of gene therapy activity in patients, and 2) replacing disease-causing RNA sequences. The disclosure provides compositions and methods for restricting the activity of an RNA trans-splicing system to specific and chosen tissues. Specifically, the disclosures provides compositions and methods for insertion of chosen RNA sequences into target RNAs using trans-splicing mediated by introns that induce cell type-specific RNA splicing patterns.

Current approaches to achieve tissue- or cell type-specific activity of gene therapies rely on the use of tissue-restricted promoters. But an ideal therapeutic would involve the high expression levels associated with non-specific promoters while maintaining tissue specificity and extend the range of tissue-restricted systems beyond those available using tissue-restricted promoters. Alternative exon choice during RNA splicing follows highly tissue-restricted patterns.

### SUMMARY

This disclosure provides a composition comprising a nucleic acid sequence encoding a trans-splicing RNA molecule comprising (a) at least one domain that promotes trans-splicing in a tissue-restricted manner (“Intronic Domain”), (b) at least one binding domain (“Antisense Domain”) that consists of a sequence complementary to a pre-mRNA present in a human cells (“Target RNA”), and (c) one coding domain that is inserted into the Target RNA via trans-splicing (“Replacement Domain”).

In some embodiments, the Antisense Domain is complementary to a gene (corresponding disease) that can cause disease in the brain and is selected from the group consisting of: SOD1, TARDBP, FUS, MATR3, SOD1, C9ORF72 (Amyotrophic lateral sclerosis); MAPT, PGRN (Frontotemporal dementia with parkinsonism); CDH23, MYO7A, USH2A (Usher's syndrome); GALC (Krabbe disease); SMPD1, NPC1, NPC2 (Niemann Pick disease); PRNP (prion disease); SCN1A (Dravet syndrome); PINK1, ATPGAP2 (early-onset Parkinson's disease); ATXN1, ATXN2, ATXN3, PLEKHG4, SPTBN2, CACNA1A, ATXN7, TTBK2, PPP2R2B, KCNC3, PRKCG, ITRP1, TBP, KCND1, FGF14 (spinocerebellar ataxics); SCN1A, SCN2A, CACNA1A, GRIN2B, GRIN2A, MECP2, FOXG1, SLC6A1, PRRT2, PTEN, KCNQ2, KCNQ3, STARD7, CLRN1 (genetic epilepsy disorders); ATM (Ataxia-telangiectasia); GLB1 (GM1 gangliosidosis); GBA (Gaucher disease); GM2A (GM2 gangliosidosis); UBE3A (Angelman syndrome); SLC2A1 (glucose transporter deficiency type 1); GLA (Fabry disease); MYOC, OPTN, TBK1, WDR36, CYPIB1 (Open Angle Glaucoma); IDUA (Hurler syndrome or Mucopolysaccharidosis 1); IDS (Hunter syndrome or Mucopolysaccharidosis 2); and CLN3 (Batten disease).

In some embodiments, the Intronic Domains promote trans-splicing primarily in brain tissue and are derived or isolated from a gene selected from the group consisting of: UBB, GFAP, STMN1, RPL13A, UCHL1, TUBB2B, ALDOA, LRRC75A-AS1, DYNLRB1, TPI1, PKM, NDRG2, MT3, RTN3, SKP1, RTN1, NRGN, DBNDD2, BSCL2, ALDOC, S100B, GNG3, FXYD6, PRDX1, HSP90AA1, SRP14, ATF4, NDUFB8, CAPZB, ATF5, ADD1, ERGIC3, GUK1, DCTN1, GSN, MORF4L1, GNAI2, SPP1, CRIP2, RNASE1, EIF4G2, PAQR6, DCTN3, CALB2, COMMD6, MEST, STXBP1, CLDND1, SH3GLB2, NREP, ABCA2, SUN2, UBXN6, DBI, CDK2AP1, NFIX, SF1, HNRNPF, GPM6B, SQSTM1, MOBP, ANAPC11, POLD2, SAMD4B, IST1, TPD52, SHD, STK25, ABR, PQBP1, PHB, SEPT9, S100A13, APBB1, RANBP1, ARHGEF2, DLGAP4, GNAO1, SEZ6L2, FDFT1, CBX1, EIF4G1, FAM213A, MAPK8IP1, PSME1, CCAR2, TKT, SYN1, OGDH, OCIAD1, SLC3A2, SLC1A5, PTTG1, DNM1, MAP6, KDM1A, PTN, FN1, CSNK1E, TMEM9, MATR3, AHCYL1, ATP6V0A1, CDIP1, CYFIP2, SLIRP, SCG5, TPX2, DMTN, SSBP1, PFKL, KIAA0930, CAMTA1, PRC1, CHTF8, TMPRSS3, BIN1, CCT8, AMZ2, TM7SF2, SEC11A, CLPTM1, AMPD2, DHX9, GAB2, CLTC, PSME3, RPP21, DYNC1I2, CDC20, NOSIP, ATP13A2, AIF1L, SURF4, PTPRN2, STK24, SDR39U1, HMOX2, UBAP2L, NRCAM, CCDC3, GPT2, TNPO2, NRXN2, PPP1R7, CAMK2A, UBE2L6, DBNL, ADAR, RNF220, MCM2, DTNA, PDIA6, PSMG2, URM1, SNX17, SV2A, SPG21, REEP2, HSD17B4, GSTO1, TRAPPC3, ZNF385A, SHISA5, FAM107B, MID1IP1, SERPINE2, CDK5RAP3, COMMD4, ANAPC15, CABP1, PTPRA, NPTN, RERE, VPS29, SPTBN2, MT1G, DTX3, GOLGA7, TPD52L2, EPB41L3, SPIN1, FARP1, NUP62, CNOT1, TMEM108, DUT, MAPRE2, PSD, NEBL, UBE2L3, KIF5C, TCF19, WDR54, CPSF7, GIT1, L1CAM, ITPR3, RHPN2, HMGA1, KCNAB2, HID1, SHF, CD99L2, C19ORF25, PAF1, B4GALT2, PTGES2, CAPG, SEMA4D, TTYH2, EXOC7, CNDP1, TACC2, BACE1, GABBR1, LMNB1, SLAIN1, GLG1, APBA2, CADM3, PC, PHLDB1, HHATL, SOGA1, CNTNAP2, RFC2, MAPK8IP3, CD200, REC8, TACC3, SYT13, SRP68, KLC4, CORO1C, ATPAF1, NARF, AP3B2, NUP93, FAM102A, SCN1B, NDUFS2, CBFA2T2, PSMA5, EPB41, and BCAR1.

In some embodiments, the Antisense Domain is complementary to a gene that can cause disease in the liver (corresponding disease) and is selected from the group consisting of: GLB1 (GM1 gangliosidosis); GBA (Gaucher disease); GM2A (GM2 gangliosidosis); PCSK9, LDLR, APOB, APOE (Familial hypercholesterolemia); GAA (Pompe disease); MYOC, OPTN, TBK1, WDR36, CYPIB1 (Open Angle Glaucoma); IDS (Hunter syndrome or Mucopolysaccharidosis 2); IDUA (Hurler syndrome or Mucopolysaccharidosis 1); CLN3 (Batten disease); F9 (Hemophilia B); and F8 (Hemophilia A).

In some embodiments, the Intronic Domains promote trans-splicing primarily in liver tissue and are derived or isolated from a gene selected from the group consisting of: ORM1, ORM2, PLG, ALDOB, CYP2C8, CPS1, ADH4, PON1, CFHR3, C6, DBI, ITIH3, ITIH4, C4BPB, PTGR1, C5, LBP, PRDX1, HPD, UBB, HPR, ADH6, RPL15, CXCL2, DPYS, HAL, SLC39A14, AQP9, SERPINA10, ADH1A, HABP2, SERPINF2, ECI2, APOM, KLKB1, ACSM5, SLCO1B3, ASGR2, IL1RAP, PSMA1, GRB14, LRRC75A-AS1, BCHE, EPHX1, DMGDH, MST1, ACSM3, SEPT2, TFR2, NDUFC1, NR1H4, AFMID, HNMT, TTC39C, RDH16, GPAM, SLC13A5, SERF2, PICALM, PCSK6, TOM1L1, ANPEP, EBNA1BP2, RCL1, DEPDC7, BTG1, KRT8, ABCA6, GJB1, KMO, HSD17B13, LEAP2, PECR, LEPR, CTNND1, ENSA, MASP2, KYNU, ABCB4, DNASE1L3, ACSL5, UBE2V1, SPTBN1, RBPMS, RAB13, IL1R1, SERPINA5, FGGY, TMEM220, ACSS2, VNN1, CTAGE5, MS4A6A, MFSD2A, SKAP1, VDAC2, SLC22A7, PPP2R5C, SORBS2, CDV3, CSAD, TRPM8, TOMM5, SLCO2B1, MICU1, NCOR1, GSAP, CBWD5, ENPP1, SPATS2L, FKBP5, NR3C1, IGF2, IGF1, PLSCR4, MYLK, SLC27A5, ALDH5A1, TXLNGY, PSMB5, MOCOS, PET100, SGK1, AZ12, TJP2, FMO4, STEAP1, DMD, RNF14, RBM34, PABPC1L, ZCCHC9, ANO6, AGMAT, MASP1, IL17RB, MRPS27, MLIP, MON2, ETF1, SULT1B1, GBP7, PRKACA, PI4K2B, SP100, TMED6, NFU1, RMDN2, IP6K2, CCNB1IP1, PCBP1-AS1, SLC39A1, and PHF11

In some embodiments, the Antisense Domain is complementary to a gene that can cause disease in the heart (corresponding disease) and is selected from the group consisting of: LAMP2 (Danon disease); GLA (Fabry disease); SLC2A1 (glucose transporter deficiency type 1); UBE3A (Angelman syndrome); MYOC, OPTN, TBK1, WDR36, CYPIB1 (Open Angle Glaucoma); IDUA (Hurler syndrome or Mucopolysaccharidosis 1); IDS (Hunter syndrome or Mucopolysaccharidosis 2); CLN3 (Batten disease); LMNA (Limb-girdle muscular dystrophy type 1B); DMD (Duchenne muscular dystrophy); DYSF (Limb-girdle muscular dystrophy type 2B); SGCB (Limb-girdle muscular dystrophy type 2E); SGCG (Limb-girdle muscular dystrophy type 2C); SGCA (Limb-girdle muscular dystrophy type 2D); SGCD (Limb-girdle muscular dystrophy type 2F); DUX4, D4Z4 (Facioscapulohumeral muscular dystrophy); F9 (Hemophilia B); F8 (Hemophilia A).

In some embodiments, the Intronic Domains promote trans-splicing primarily in heart tissue and are derived or isolated from a gene selected from the group consisting of: TNNI3, TNNT2, MB, FHL2, CSRP3, FHL1, UBB, MYOM2, CD36, GSN, PDHA1, CRIP2, POPDC2, SORBS2, CPT1B, RYR2, ANXA11, ITGA7, SORBS1, NDUFC1, NEXN, MYH7B, ABLIM1, NEBL, CAST, NME2, CYB5R3, TRDN, TACC2, NDUFA5, MYL12B, FHOD3, CALCOCO2, NCAM1, GPD1L, SGCA, AQP1, AKAP1, ARL6IP4, LRRC39, TTN, TSC22D1, PPP1R12B, OBSL1, MYO15B, PKIG, HMOX2, CDK18, BTNL9, ITGB1BP2, SEPT2, CAV2, DTNA, C15orf41, ATP1A1, BCO2, MYL4, ADCY5, CLIC5, SLC8A1, RRAD, AKAP13, AK3, MYO1C, FGF12, LAMA5, ADAM15, WNK2, SUN1, KLHL7, RNF207, HIF3A, HSPG2, LPCAT4, ADH1B, ENY2, RGS3, FRMD3, CCNB1IP1, PHC2, STAB1, HAND2-AS1, PTPN3, PARVB, PKD1, SPEG, SYNE2, ZNF638, SEPT4, DAG1, SLC12A7, FBXO18, FAM107A, RBM17, MYPN, GRIP2, SCN5A, WFS1, RABGAP1L, SYNE1, HLA-DRB5, SNRPN, MEG3, RAPGEF3, SEC13, C1QTNF1, UCKL1, FRY, IP6K2, CUX1, ANK3, MYH14, ADCY4, C3orf18, MYLK3, PXN, TSPAN18, PDE1C, C6orf48, BCS1L, ATG4D, HRAS, TENM2, PLXNB1, N4BP2L2, TNXB, LTBP1, MAPK8IP3, COL1A1, GOAT, ACOT11, MTUS2, VPS45, and KCNIP2.

In some embodiments, the Antisense Domain is complementary to a gene that can cause disease in the retina (corresponding disease) and is selected from the group consisting of: USHA2A, RPGR, RP2, RHO, PRPF31, USH1F, PRPF3, PRPF6 (Retinitis pigmentosa)

In some embodiments, the Intronic Domains promote trans-splicing primarily in retinal tissue and are derived or isolated from a gene selected from the group consisting of: ADGRV1, AMPH, ATP1B2, BSG, CASK, CC2D2A, CEP290, CLTC, DMD, DOC2B, EFR3A, ERP44, FARP2, FN3KRP, GRAMD1B, GSS, HCN1, IFT140, IMPDH1, INTS13, KCTD5, KIF1B, KIF1B, KMT2D, MADD, MAN2A2, MARCH1, MSI2, NAP1L4, PPP3CC, PROM1, PTPRF, PTPRF, RIMS2, SLC17A7, SLC4A7, STXBP5, TTC8, UNC13B, UNC13B, WDR47, and YKT6.

In some embodiments, the Antisense Domain is complementary to a gene that can cause disease in muscle (corresponding disease) and is selected from the group consisting of: LAMP2 (Danon disease); GLA (Fabry disease); SLC2A1 (glucose transporter deficiency type 1); UBE3A (Angelman syndrome); MYOC, OPTN, TBK1, WDR36, CYPIB1 (Open Angle Glaucoma); IDUA (Hurler syndrome or Mucopolysaccharidosis 1); IDS (Hunter syndrome or Mucopolysaccharidosis 2); CLN3 (Batten disease); LMNA (Limb-girdle muscular dystrophy type 1B); DMD (Duchenne muscular dystrophy); DYSF (Limb-girdle muscular dystrophy type 2B); SGCB (Limb-girdle muscular dystrophy type 2E); SGCG (Limb-girdle muscular dystrophy type 2C); SGCA (Limb-girdle muscular dystrophy type 2D); SGCD (Limb-girdle muscular dystrophy type 2F); DUX4, D4Z4 (Facioscapulohumeral muscular dystrophy); F9 (Hemophilia B); and F8 (Hemophilia A)

In some embodiments, the Intronic Domains promote trans-splicing primarily in muscle tissue and are derived or isolated from a gene selected from the group consisting of: ALDOA, TNNT3, MYBPC1, ATP2A1, NEB, STAC3, CMYA5, MYH7, TNNI1, CA3, ASB5, PLIN2, TRDN, OBSCN, RYR1, RAP1A, SPARCL1, CSNK1A1, NPEPPS, AKR1C3, IDI2, MBNL1, FNDC5, TMOD4, ATP6V1F, DAP3, KPNB1, NOMO2, BEST3, HP1BP3, SEPT2, TIMM23, MYPN, DHRS7C, PALLD, POLR2J3, KANSL3, THOC7, MEF2C, NOMO1, YWHAZ, ZBTB16, SPAG9, CCZ1, MYLK2, ASAH1, GTF2I, CCPG1, SURF4, SMARCA2, CLTCL1, SLC6A6, CHRNB1, MAFG, COMMD6, SLC7A8, CDK11B, and DNASE1L1.

In some embodiments, the Antisense Domain is complementary to a gene (corresponding accession numbers in brackets, corresponding disease in parentheses) that encodes a Target RNA that sometimes carry disease-causing mutations and is selected from the group consisting of: TNFRSF13B [ENSG00000240505] (common variable immune deficiency); ADA, CECR1 [ENSG00000196839, ENSG00000093072] (Adenosine deaminase deficiency); IL2RG [ENSG00000147168] (X-linked severe combined immunodeficiency); HBB [ENSG00000244734] (Beta-thassalemia); HBA1, HBA2 [ENSG00000206172, ENSG00000188536] (alpha-thassalemia); U2AF1 [ENSG00000160201] (myelodysplastic syndrome); SOD1, TARDBP, FUS, MATR3, SOD1, C9ORF72 [ENSG00000142168, ENSG00000120948, ENSG00000089280, ENSG00000015479, ENSG00000142168, ENSG00000147894] (Amyotrophic lateral sclerosis); MAPT, PGRN [ENSG00000186868, ENSG00000030582] (Frontotemporal dementia with parkinsonism); CDH23, MYO7A, USH2A [ENSG00000107736, ENSG00000137474, ENSG00000042781] (Usher's syndrome); GALC [ENSG00000054983] (Krabbe disease); SMPD1, NPC1, NPC2 [ENSG00000166311, ENSG00000141458, ENSG00000119655] (Niemann Pick disease); PRNP [ENSG00000171867] (prion disease); SCN1A [ENSG00000144285] (Dravet syndrome); PINK1, ATPGAP2 [ENSG00000158828] (early-onset Parkinson's disease); ATXN1, ATXN2, ATXN3, PLEKHG4, SPTBN2, CACNA1A, ATXN7, TTBK2, PPP2R2B, KCNC3, PRKCG, ITPR1, TBP, KCND1, FGF14 [ENSG00000124788, ENSG00000204842, ENSG00000066427, ENSG00000196155, ENSG00000173898, ENSG00000141837, ENSG00000163635, ENSG00000128881, ENSG00000156475, ENSG00000131398, ENSG00000126583, ENSG00000150995, ENSG00000112592, ENSG00000102057, ENSG00000102466] (spinocerebellar ataxias); SCN1A, SCN2A, CACNA1A, GRIN2B, GRIN2A, MECP2, FOXG1, SLC6A1, PRRT2, PTEN, KCNQ2, KCNQ3, STARD7, CLRN1 [ENSG00000144285, ENSG00000136531, ENSG00000141837, ENSG00000273079, ENSG00000183454, ENSG00000169057, ENSG00000176165, ENSG00000157103, ENSG00000167371, ENSG00000171862, ENSG00000075043, ENSG00000184156, ENSG00000084090, ENSG00000163646] (genetic epilepsy disorders); ATM [ENSG00000149311] (Ataxia-telangiectasia); GLB1 [ENSG00000170266] (GM1 gangliosidosis); GBA [ENSG00000177628] (Gaucher disease); GM2A [ENSG00000196743] (GM2 gangliosidosis); UBE3A [ENSG00000114062] (Angelman syndrome); SLC2A1 [ENSG00000117394] (glucose transporter deficiency type 1); LAM P2 [ENSG00000005893] (Danon disease); GLA [ENSG00000102393] (Fabry disease); PKD1, PKD2 [ENSG00000008710, ENSG00000118762] (Autosomal dominant polycystic kidney disease); GAA [ENSG00000171298] (Pompe disease); PCSK9, LDLR, APOB, APOE [ENSG00000169174, ENSG00000130164, ENSG00000084674, ENSG00000130203] (Familial hypercholesterolemia); MYOC, OPTN, TBK1, WDR36, CYPIB1 [ENSG00000034971, ENSG00000123240, ENSG00000183735, ENSG00000134987, ENSG00000138061] (Open Angle Glaucoma); IDUA [ENSG00000127415] (Hurler syndrome or Mucopolysaccharidosis 1); IDS [ENSG00000010404] (Hunter syndrome or Mucopolysaccharidosis 2); CLN3 [ENSG00000188603] (Batten disease); DMD [ENSG00000198947] (Duchenne muscular dystrophy); LMNA [ENSG00000160789] (Limb-girdle muscular dystrophy type 1B); DYSF [ENSG00000135636] (Limb-girdle muscular dystrophy type 2B); SGCA [ENSG00000108823] (Limb-girdle muscular dystrophy type 2D); SGCB [ENSG00000163069] (Limb-girdle muscular dystrophy type 2E); SGCG [ENSG00000102683] (Limb-girdle muscular dystrophy type 2C); SGCD [ENSG00000170624] (Limb-girdle muscular dystrophy type 2F); DUX4 [ENSG00000260596] (Facioscapulohumeral muscular dystrophy); F9 [ENSG00000101981] (Hemophilia B); F8 [ENSG00000185010] (Hemophilia A); USHA2A, RPGR, RP2, RHO, PRPF31, USH1F, PRPF3, PRPF6 [ENSG00000156313, ENSG00000102218, ENSG00000163914, ENSG00000105618, ENSG00000150275, ENSG00000117360, ENSG00000101161] (Retinitis pigmentosa); CFTR [ENSG00000001626] (cystic fibrosis); GJB2, GJB6, STRC, DFNA1, WFS1 [ENSG00000165474, ENSG00000121742, ENSG00000242866, ENSG00000131504, ENSG00000109501] (autosomal dominant hearing impairment); POU3F3 [ENSG00000198914] (nonsyndromic hearing loss).

In some embodiments, the Replacement domain is derived or isolated from the target RNA.

In some embodiments, the Intronic Domains carry binding sites that a preferentially-targeted by RNA-binding proteins with disease-causing mutations. In some embodiments, the dissociation constant of these mutated RNA-binding proteins and the Intronic Domain is lower than the dissociation constant of the non-mutated RNA-binding protein and the Intronic Domain.

In some embodiments, the Intronic Domains carry binding sites for RNA-binding proteins that promote trans-splicing primarily in 1, 2, 3, 4, 5, 6, 7, 8, 9, 10 or more tissues but not other tissues. In some embodiments, the RNA-binding protein binding sites in the Intronic Domain are targeted by RNA-binding proteins selected from the group consisting of: DDX39, DDX3X, DDX3Y, DDX41, DDX5, DHX16, DHX8, DHX9, INTS6, DDX48, HNRNPA0, HNRNPA1, HNRNPA2B1, HNRPA3, HNRPAB, HNRPC, HNRPDL, HNRPF, HNRPH1, HNRPH2, HNRPLL, HNRPM, HNRPUL1, PTBP1, PTBP2, RBMXL2, SYNCRIP, CLK3, CLK4, SRPK1, SRPK2, SRPK3, ARS2, BUB3, C7ORF30, CIRBP, CRNKL1, CSDA, DNAJC8, EEF1A1, EIF3S10, FMR1, GRSF1, GTL3, IGF2BP3, IK, ILF2, ILF3, MATR3, MBNL1, MBNL2, MBNL3, MOV10, MSI1, MSI2, MYEF2, NHP2L1, PABPC1, PCBP1, PCBP2, PCBP4, PPIE, PPIL2, PPIL2, PPM1G, PPWD1, PRPF6, PSEN1, RALY, RBM15, RBM9, RBMS1, RBMX, RDBP, ROD1, SSB, THOC3, THOC4, TNPO1, TOP1MT, ZFP207, EFTUD2, LSM1, LSM2, LSM3, LSM4, LSM7, LSM8, NA, SART1, SF3A1, SF3A2, SF3A3, SF3B1, SF3B2, SNRPA, SNRPA1, SNRPB, SNRPC, SNRPD1, SNRPD2, SNRPG, SNRPN, TXNL4, U2AF1L1, U2AF1L2, USP39, WDR57, PRPF31, PRPF4, PRPF40A, RBM17, NA, A2BP1, ACIN1, ASCC3L1, BCAS2, BRUNOL4, BUD13, BUD31, CD2BP2, CDC40, CDC5L, CRKRS, CUGBP2, ELAVL1, ELAVL1, ELAVL1, ELAVL2, ELAVL3, ELAVL4, FUSIP1, HSPA1B, HSPAS, HSPA8, NCBP2, NONO, NOVA1, NUDT21, PLRG1, PRPF19, PSIP1, RBM8A, RBMS1, SMU1, SNW1, TCERG1, TIA1, TIAL1, TNRC4, WBP11, WTAP, XAB2, YBX1, SFRS1, SFRS10, SFRS16, SFRS3, SFRS4, and SFRS6

In some embodiments, the trans-splicing RNA further comprises a 5′ untranslated region. In some embodiments, the 5′ untranslated region increases the stability of the trans-splicing nucleic acid. In some embodiments, the 5′ untranslated region reduces the stability of the trans-splicing nucleic acid. In some embodiments, the 5′ untranslated region alters the localization of the trans-splicing nucleic acid. In some embodiments, the 5′ untranslated region alters the processing of the trans-splicing nucleic acid.

In some embodiments, the trans-splicing RNA further comprises a 3′ untranslated region. In some embodiments, the 3′ untranslated region increases the stability of the trans-splicing nucleic acid. In some embodiments, the 3′ untranslated region reduces the stability of the trans-splicing nucleic acid. In some embodiments, the 3′ untranslated region alters the localization of the trans-splicing nucleic acid. In some embodiments, the 3′ untranslated region alters the processing of the trans-splicing nucleic acid.

In some embodiments, the intronic domains promote trans-splicing of the trans-splicing nucleic acid in a spatially-restricted manner so that trans-splicing occurs primarily in one tissue selected from the group consisting of: muscle cells, cardiac muscle cells, neurons, kidney cells, liver cells, epithelial cells, striatal tissue, basal ganglia tissue, substantia nigra tissue, lung tissue, spinal cord tissue, cranial nerve tissue, optic nerve tissue, oligodendrocytes, astrocytes, ependymal cells, microglia, airway tissue, inner eye tissue, retinal tissue, cerebellar tissue, pineal gland, thymus gland, pituitary gland, thyroid gland, adrenal gland, apocrine gland, holocrine gland, merocrine gland, serous gland, mucous gland, sebaceous gland, naïve T cells, effector T cells, helper T cells, memory T cells, regulatory T cells, gamma-delta T cells, natural killer cells, B cells, dendrocytes and macrophages.

In some embodiments, the Intronic Domain promote trans-splicing primarily in 2, 3, 4, 5, 6, 7, 8, 9, 10, 11, 12, 13, 14, 15, 16, 17, 18, 19 or more tissues or cell types but not trans-spliced in other cell types or tissues.

In some embodiments, the Intronic Domains broadly promote trans-splicing in many tissues but do not promote trans-splicing in a tissue selected from the following group consisting of: skeletal muscle cells, cardiac muscle cells, neurons, kidney cells, liver cells, epithelial cells, striatal tissue, basal ganglia tissue, substantia nigra tissue, lung tissue, spinal cord tissue, cranial nerve tissue, optic nerve tissue, oligodendrocytes, astrocytes, ependymal cells, microglia, airway tissue, inner eye tissue, retinal tissue, cerebellar tissue, pineal gland, thymus gland, pituitary gland, thyroid gland, adrenal gland, apocrine gland, holocrine gland, merocrine gland, serous gland, mucous gland, sebaceous gland, naïve T cells, effector T cells, helper T cells, memory T cells, regulatory T cells, gamma-delta T cells, natural killer cells, B cells, dendrocytes and macrophages. In some embodiments, the Intronic Domains do not promote trans-splicing in 2, 3, 4, 5, 6, 7, 8, 9, 10, 11, 12, 13, 14, 15, 16, 17, 18, 19 or more tissues but promote trans-splicing in other cell types or tissues.

In some embodiments, the Replacement Domain is comprised of sequence derived or isolated from a human gene. In some embodiments of the compositions of the disclosure, the sequence comprising the Replacement Domain has at least 50%, 55%, 60%, 65%, 70%, 75%, 80%, 87%, 90%, 95%, 97%, 99% or any percentage in between of identity with a human gene. In some embodiments, the Replacement Domain has 100% identity with a sequence derived or isolated from a human gene. In some embodiments, the Replacement Domain comprises or consists of 2 nucleotides, 5 nucleotides, 10 nucleotides, 20 nucleotides, 30 nucleotides, 40 nucleotides, 50 nucleotides, 60 nucleotides, 70 nucleotides, 80 nucleotides, 90 nucleotides, 100 nucleotides, 110 nucleotides, 120 nucleotides, 130 nucleotides, 140 nucleotides, 150 nucleotides, 160 nucleotides, 170 nucleotides, 180 nucleotides, 190 nucleotides, 200 nucleotides, 210 nucleotides, 220 nucleotides, 230 nucleotides, 240 nucleotides, 250 nucleotides, 260 nucleotides, 270 nucleotides, more than 270 nucleotides, or any number of nucleotides in between.

In some embodiments of the compositions of the disclosure, the sequence comprising the Antisense Domain has at least 50%, 55%, 60%, 65%, 70%, 75%, 80%, 87%, 90%, 95%, 97%, 99% or any percentage in between of complementarity to the Target RNA sequence. In some embodiments, the Antisense Domain has 100% complementarity to the Target RNA sequence. In some embodiments, the Antisense Domain comprises or consists of 20 nucleotides, 30 nucleotides, 40 nucleotides, 50 nucleotides, 60 nucleotides, 70 nucleotides, 80 nucleotides, 90 nucleotides, 100 nucleotides, 110 nucleotides, 120 nucleotides, 130 nucleotides, 140 nucleotides, 150 nucleotides, 160 nucleotides, 170 nucleotides, 180 nucleotides, 190 nucleotides, 200 nucleotides, 210 nucleotides, 220 nucleotides, 230 nucleotides, 240 nucleotides, 250 nucleotides, 260 nucleotides, 270 nucleotides, more than 270 nucleotides, or any number of nucleotides in between the complementary to the Target RNA sequence.

In some embodiments of the compositions of the disclosure, the sequence comprising the trans-splicing RNA further comprises a sequence encoding a promoter capable of expressing the trans-splicing RNA in a eukaryotic cell.

In some embodiments of the compositions of the disclosure, the eukaryotic cell is an animal cell. In some embodiments, the animal cell is a mammalian cell. In some embodiments, the animal cell is a human cell.

In some embodiments of the compositions and methods of the disclosure, a vector of the disclosure is a viral vector. In some embodiments, the viral vector comprises a sequence isolated or derived from a retrovirus. In some embodiments, the viral vector comprises a sequence isolated or derived from a lentivirus. In some embodiments, the viral vector comprises a sequence isolated or derived from an adenovirus. In some embodiments, the viral vector comprises a sequence isolated or derived from an adeno-associated virus (AAV). In some embodiments, the viral vector is replication incompetent. In some embodiments, the viral vector is isolated or recombinant. In some embodiments, the viral vector is self-complementary.

In some embodiments of the compositions and methods of the disclosure, the viral vector comprises a sequence isolated or derived from an adeno-associated virus (AAV). In some embodiments, the viral vector comprises an inverted terminal repeat sequence or a capsid sequence that is isolated or derived from an AAV of serotype AAV1, AAV2, AAV3, AAV4, AAV5, AAV6, AAV7, AAV8, AAV9, AAV10, AAV11 or AAV12. In some embodiments, the viral vector is replication incompetent. In some embodiments, the viral vector is isolated or recombinant (rAAV). In some embodiments, the viral vector is self-complementary (scAAV).

In some embodiments of the compositions and methods of the disclosure, a vector of the disclosure is a non-viral vector. In some embodiments, the vector comprises or consists of a nanoparticle, a micelle, a liposome or lipoplex, a polymersome, a polyplex, an exosome or a dendrimer. In some embodiments, the vector is an expression vector or recombinant expression system. As used herein, the term “recombinant expression system” refers to a genetic construct for the expression of certain genetic material formed by recombination.

In some embodiments of the compositions and methods of the disclosure, an expression vector, viral vector or non-viral vector provided herein, includes without limitation, an expression control element. An “expression control element” as used herein refers to any sequence that regulates the expression of a coding sequence, such as a gene. Exemplary expression control elements include but are not limited to promoters, enhancers, microRNAs, post-transcriptional regulatory elements, polyadenylation signal sequences, 5′ or 3′ untranslated regions, and introns.

Expression control elements may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. In some embodiments, expression control by a promoter is tissue-specific. Non-limiting exemplary promoters include CMV, CBA, CAG, Cbh, EF-1a, PGK, UBC, GUSB, UCOE, hAAT, TBG, Desmin, MCK, C5-12, NSE, Synapsin, PDGF, MecP2, CaMKII, mGluR2, NFL, NFH, n132, PPE, ENK, EAAT2, GFAP, MBP, H1 and U6 promoters. In some embodiments, the promoter is a sequence isolated or derived from a promoter capable of driving expression of a transfer RNA (tRNA). In some embodiments, the promoter is isolated or derived from an alanine tRNA promoter, an arginine tRNA promoter, an asparagine tRNA promoter, an aspartic acid tRNA promoter, a cysteine tRNA promoter, a glutamine tRNA promoter, a glutamic acid tRNA promoter, a glycine tRNA promoter, a histidine tRNA promoter, an isoleucine tRNA promoter, a leucine tRNA promoter, a lysine tRNA promoter, a methionine tRNA promoter, a phenylalanine tRNA promoter, a proline tRNA promoter, a serine tRNA promoter, a threonine tRNA promoter, a tryptophan tRNA promoter, a tyrosine tRNA promoter, or a valine tRNA promoter. In some embodiments, the promoter is isolated or derived from a valine tRNA promoter.

An “enhancer” is a region of DNA that can be bound by activating proteins to increase the likelihood or frequency of transcription. Non-limiting exemplary enhancers and post-transcriptional regulatory elements include the CMV enhancer and WPRE.

In some embodiments of the compositions and methods of the disclosure, an expression vector, viral vector or non-viral vector provided herein, includes without limitation, vector elements such as an IRES or 2A peptide sites for configuration of “multicistronic” or “polycistronic” or “bicistronic” or tricistronic” constructs, i.e., having double or triple or multiple coding areas or exons, and as such will have the capability to express from mRNA two or more proteins from a single construct. Multicistronic vectors simultaneously express two or more separate proteins from the same mRNA. The two strategies most widely used for constructing multicistronic configurations are through the use of an IRES or a 2A self-cleaving site. An “IRES” refers to an internal ribosome entry site or portion thereof of viral, prokaryotic, or eukaryotic origin which are used within polycistronic vector constructs. In some embodiments, an IRES is an RNA element that allows for translation initiation in a cap-independent manner. The term “self-cleaving peptides” or “sequences encoding self-cleaving peptides” or “2A self-cleaving site” refer to linking sequences which are used within vector constructs to incorporate sites to promote ribosomal skipping and thus to generate two polypeptides from a single promoter, such self-cleaving peptides include without limitation, T2A, and P2A peptides or sequences encoding the self-cleaving peptides.

In some embodiments, the vector is a viral vector. In some embodiments, the vector is an adenoviral vector, an adeno-associated viral (AAV) vector, or a lentiviral vector. In some embodiments, the vector is a retroviral vector, an adenoviral/retroviral chimera vector, a herpes simplex viral I or II vector, a parvoviral vector, a reticuloendotheliosis viral vector, a polioviral vector, a papillomaviral vector, a vaccinia viral vector, or any hybrid or chimeric vector incorporating favorable aspects of two or more viral vectors. In some embodiments, the vector further comprises one or more expression control elements operably linked to the polynucleotide. In some embodiments, the vector further comprises one or more selectable markers. In some embodiments, the AAV vector has low toxicity. In some embodiments, the AAV vector does not incorporate into the host genome, thereby having a low probability of causing insertional mutagenesis. In some embodiments, the AAV vector can encode a range of total polynucleotides from 0.3 kb to 4.75 kb. In some embodiments, exemplary AAV vectors that may be used in any of the herein described compositions, systems, methods, and kits can include an AAV1 vector, a modified AAV1 vector, an AAV2 vector, a modified AAV2 vector, an AAV3 vector, a modified AAV3 vector, an AAV4 vector, a modified AAV4 vector, an AAV5 vector, a modified AAV5 vector, an AAV6 vector, a modified AAV6 vector, an AAV7 vector, a modified AAV7 vector, an AAV8 vector, an AAV9 vector, an AAV.rh10 vector, a modified AAV.rh10 vector, an AAV.rh32/33 vector, a modified AAV.rh32/33 vector, an AAV.rh43 vector, a modified AAV.rh43 vector, an AAV.rh74 vector, a modified AAV.rh74 vector, an AAV.rh64R1 vector, and a modified AAV.rh64R1 vector and any combinations or equivalents thereof. In some embodiments, the lentiviral vector is an integrase-competent lentiviral vector (ICLV). In some embodiments, the lentiviral vector can refer to the transgene plasmid vector as well as the transgene plasmid vector in conjunction with related plasmids (e.g., a packaging plasmid, a rev expressing plasmid, an envelope plasmid) as well as a lentiviral-based particle capable of introducing exogenous nucleic acid into a cell through a viral or viral-like entry mechanism. Lentiviral vectors are well-known in the art (see, e.g., Trono D. (2002) Lentiviral vectors, New York: Spring-Verlag Berlin Heidelberg and Durand et al. (2011) Viruses 3(2):132-159 doi: 10.3390/v3020132). In some embodiments, exemplary lentiviral vectors that may be used in any of the herein described compositions, systems, methods, and kits can include a human immunodeficiency virus (HIV) 1 vector, a modified human immunodeficiency virus (HIV) 1 vector, a human immunodeficiency virus (HIV) 2 vector, a modified human immunodeficiency virus (HIV) 2 vector, a sooty mangabey simian immunodeficiency virus (SIVSM) vector, a modified sooty mangabey simian immunodeficiency virus (SIVSM) vector, a African green monkey simian immunodeficiency virus (SIVAGM) vector, a modified African green monkey simian immunodeficiency virus (SIVAGM) vector, an equine infectious anemia virus (EIAV) vector, a modified equine infectious anemia virus (EIAV) vector, a feline immunodeficiency virus (FIV) vector, a modified feline immunodeficiency virus (FIV) vector, a Visna/maedi virus (VNV/VMV) vector, a modified Visna/maedi virus (VNV/VMV) vector, a caprine arthritis-encephalitis virus (CAEV) vector, a modified caprine arthritis-encephalitis virus (CAEV) vector, a bovine immunodeficiency virus (BIV), or a modified bovine immunodeficiency virus (BIV).

In some embodiments, the trans-splicing nucleic acid is RNA, DNA, a DNA/RNA hybrid, and/or comprises at least one of a nucleic acid analog, a chemically-modified nucleic acid, or a chimera composed of two or more nucleic acids or nucleic acid analogs. As used herein, the term “nucleic acid analog” refers to a compound having structural similarity to a canonical purine or pyrimidine base occurring in DNA or RNA. The nucleic acid analog may contain a modified sugar and/or a modified nucleobase, as compared to a purine or pyrimidine base occurring naturally in DNA or RNA. In some embodiments, the nucleic acid analog is a 2′-deoxyribonucleoside, 2′-ribonucleoside, 2′-deoxyribonucleotide or a 2′-ribonucleotide, wherein the nucleobase includes a modified base (such as, for example, xanthine, uridine, oxanine (oxanosine), 7-methlguanosine, dihydrouridine, 5-methylcytidine, C3 spacer, 5-methyl dC, 5-hydroxybutynl-2′-deoxyuridine, 5-nitroindole, 5-methyl iso-deoxycytosine, iso deoxyguanosine, deoxyuradine, iso deoxycytidine, other 0-1 purine analogs, N-6-hydroxylaminopurine, nebularine, 7-deaza hypoxanthine, other 7-deazapurines, and 2-methyl purines). In some embodiments, the nucleic acid analog may be selected from the group consisting of inosine, 7-deaza-2′-deoxyinosine, 2′-aza-2′-deoxyinosine, PNA-inosine, morpholino-inosine, LNA-inosine, phosphoramidate-inosine, 2′-O-methoxyethyl-inosine, and 2′-OMe-inosine. In other embodiments the nucleic acid analog is a nucleic acid mimic (such as, for example, artificial nucleic acids and xeno nucleic acids (XNA).

### BRIEF DESCRIPTION OF THE DRAWINGS

FIG. 1 is an illustration of the unmet need addressed by the present invention. FIG. 1A indicates that human disease can affect some tissue and not others, or can primarily occur in some tissues compared to others. FIG. 1B demonstrates that therapeutic activity in a patient typically occurs in not only affected tissues but also unaffected tissues. FIG. 1C demonstrates the present invention which limits the activity of a trans-splicing therapeutic to the target tissue and not others.

FIG. 2 describes an example embodiment of the present invention along with a schematic to illustrate the activity. FIG. 2A describes, in general, human disease caused by genetic mutations. FIG. 2B describes a example embodiment of the present invention that involves 2 antisense domains, 2 intronic domains, and one replacement domain. FIG. 3C describes the tissue-restricted activity of the present invention so that trans-splicing occurs only in the target tissue resulting in replacement of the defective RNA sequence (top box).

FIG. 3 is a non-limiting illustration of the origin and structure of intronic domains in some embodiments of the present invention. FIG. 3A illustrates intronic domains that are taken from a gene that experiences tissue-specific alternative splicing of an adjacent exon. FIG. 3B illustrates intronic domains that are composed of combinations of introns derived from genes that experience tissue-specific alternative splicing of adjacent alternative exons.

FIG. 3C illustrates intronic domains that are composed of combinations of sequences that comprise binding sites for RNA-binding proteins that promote tissue-specific splicing patterns.

FIG. 4 provides a schematic of an experiment that results in identification of intronic domains that promote tissue- or cell type-specific trans-splicing. FIG. 4A illustrates that two functional components of the experiment: a reporter that reveals tissue-specific trans-splicing activity and a trans-splicing RNA that carries the candidates intronic domains. The reporter carries a promoter and incomplete coding sequence for GFP (N-terminal and C-terminal portions of GFP or “N-GFP” and “C-GFP”) that requires the internal section of GFP from the trans-splicing reaction to form a complete and fluorescent GFP protein. The trans-splicing RNA carries this internal GFP section (“int-GFP”) that is flanked by intronic domains and antisense domains. When the reporter is transfected into a human cell, there is little or no GFP signal (FIG. 4B, top). When the reporter and trans-splicing RNA are co-transfected in a non-target cell type, there is similarly little or no GFP signal (FIG. 4B, middle). When the reporter and trans-splicing RNA are co-transfected in a target cell type, GFP signal is produced (FIG. 4B, bottom). In this manner, intronic domains that promote trans-splicing in target cell types or tissues but not others can be identified.

FIG. 5 provides a schematic of an experiment that results in identification of trans-splicing systems that reverse the molecular caused of disease LGMD1B specifically in muscle tissue. FIG. 5A describes the LGMD1B reporter that carries that characteristic mutation near exon 9 of gene LM NA that promotes inclusion of exon 9 and resulting premature termination of the coding sequence. To reverse this disease-causing mutation, the trans-splicing RNA (FIG. 5A, bottom) targets exon 9 and replaces it with a short replacement domain that does not contain a stop codon (FIG. 5C, bottom). In a non-target cell type, the system is not active (FIG. 5B, top). In this manner, a high-quality trans-splicing system that can efficiently replace this defective sequence in LMNA can be identified.

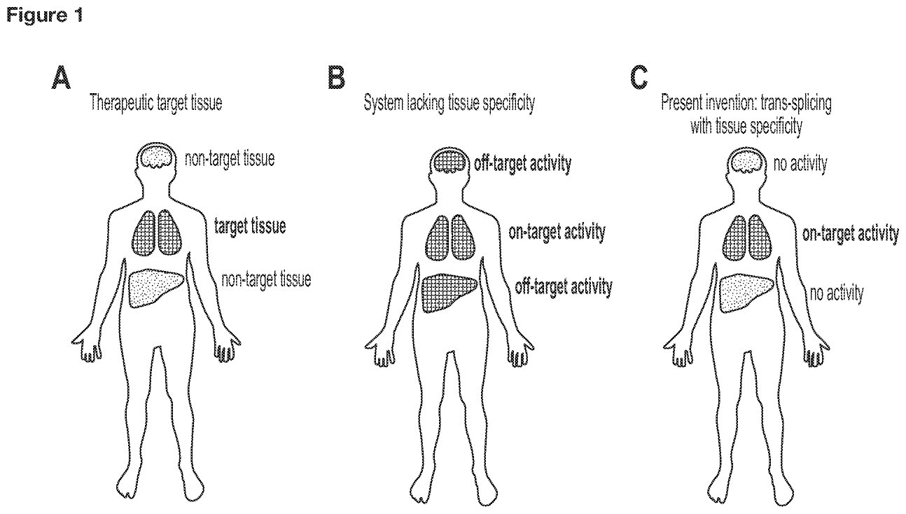

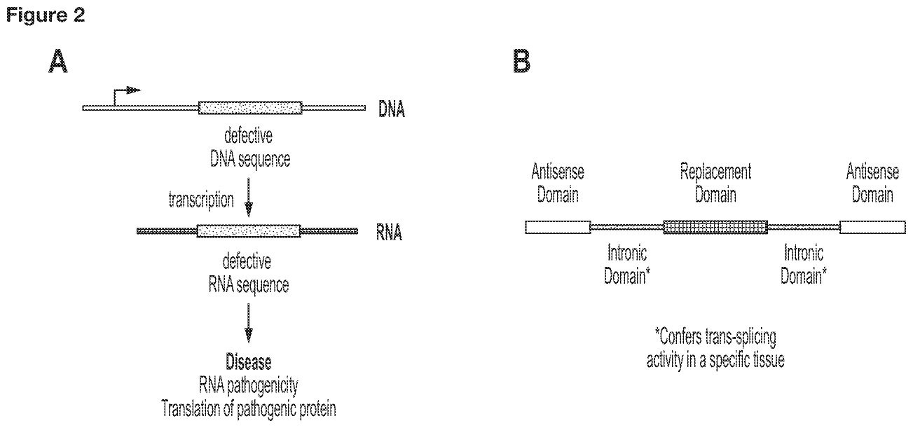

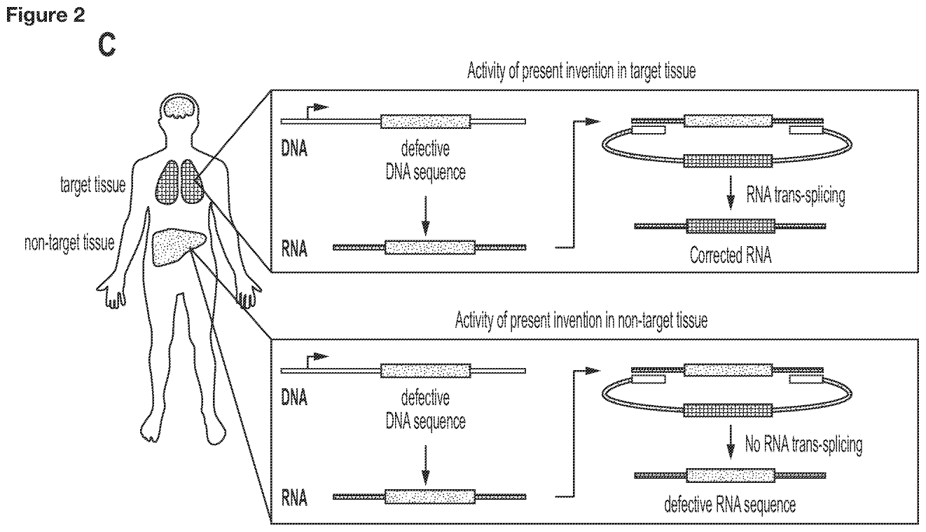

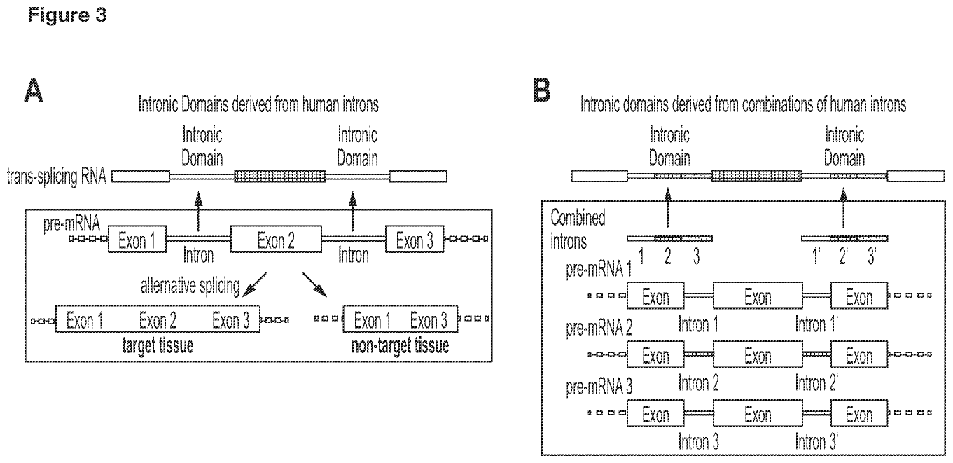

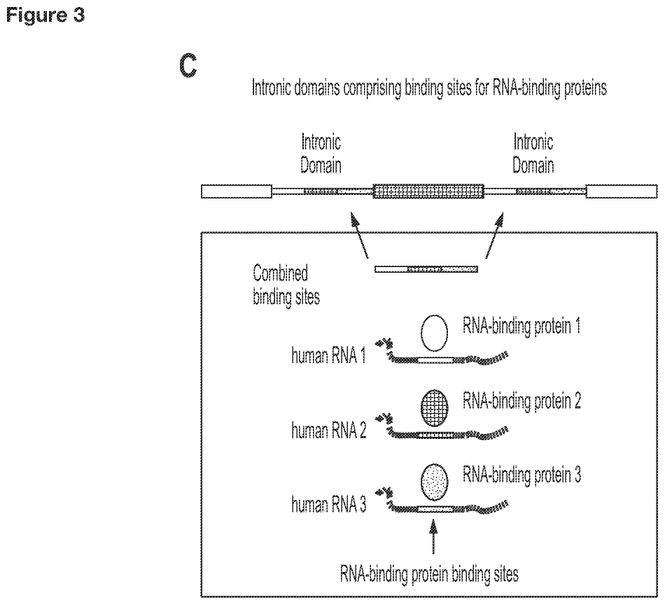

### DETAILED DESCRIPTION

This disclosure provides a therapeutic combination of replacement of disease-causing RNA sequences and tissue-restricted therapeutic activity. Specifically, the disclosure provides compositions and methods for specifically targeting disease-causing RNA molecules in specific, chosen tissues and replacing disease-causing RNA sequences within these RNA molecules. In particular, the disclosure provides a composition comprising a nucleic acid sequence encoding a trans-splicing therapeutic systems comprising (a) at least one domain that promotes trans-splicing in a tissue-restricted manner, (b) at least one binding domain that consists of a sequence complementary to a pre-mRNA present in a human cells, and (c) one coding domain that is inserted into the Target RNA via trans-splicing, wherein the target RNA causes disease, and wherein the intronic domain promotes trans-splicing specifically in chosen tissue but not others, and wherein the replacement domain encodes a non-mutated sequence. This disclosure provides vectors, compositions and cells comprising the trans-splicing compositions and methods of using the trans-splicing compositions.

### Intronic Domains

Compositions comprising intronic domains disclosed herein include any sequences that promote trans-splicing in a manner restricted to specific cell or tissue types or groups of cell or tissue types.

Compositions comprising intronic domains disclosed herein include any sequence that promotes trans-splicing primarily in brain tissue. In some embodiments, the intronic domains are derived or isolated from human genes that experience brain-specific alternative splicing events. Exemplary intronic domains that promote brain-specific trans-splicing include without limitation genes such as: UBB, GFAP, STMN1, RPL13A, UCHL1, TUBB2B, ALDOA, LRRC75A-AS1, DYNLRB1, TPI1, PKM, NDRG2, MT3, RTN3, SKP1, RTN1, NRGN, DBNDD2, BSCL2, ALDOC, S100B, GNG3, FXYD6, PRDX1, HSP90AA1, SRP14, ATF4, NDUFB8, CAPZB, ATF5, ADD1, ERGIC3, GUK1, DCTN1, GSN, MORF4L1, GNAI2, SPP1, CRIP2, RNASE1, EIF4G2, PAQR6, DCTN3, CALB2, COMMD6, MEST, STXBP1, CLDND1, SH3GLB2, NREP, ABCA2, SUN2, UBXN6, DBI, CDK2AP1, NFIX, SF1, HNRNPF, GPM6B, SQSTM1, MOBP, ANAPC11, POLD2, SAMD4B, IST1, TPD52, SHD, STK25, ABR, PQBP1, PHB, SEPT9, S100A13, APBB1, RANBP1, ARHGEF2, DLGAP4, GNAO1, SEZ6L2, FDFT1, CBX1, EIF4G1, FAM213A, MAPK8IP1, PSME1, CCAR2, TKT, SYN1, OGDH, OCIAD1, SLC3A2, SLC1A5, PTTG1, DNM1, MAP6, KDM1A, PTN, FN1, CSNK1E, TMEM9, MATR3, AHCYL1, ATP6V0A1, CDIP1, CYFIP2, SLIRP, SCG5, TPX2, DMTN, SSBP1, PFKL, KIAA0930, CAMTA1, PRC1, CHTF8, TMPRSS3, BIN1, CCT8, AMZ2, TM7SF2, SEC11A, CLPTM1, AMPD2, DHX9, GAB2, CLTC, PSME3, RPP21, DYNC1I2, CDC20, NOSIP, ATP13A2, AIF1L, SURF4, PTPRN2, STK24, SDR39U1, HMOX2, UBAP2L, NRCAM, CCDC3, GPT2, TNPO2, NRXN2, PPP1R7, CAMK2A, UBE2L6, DBNL, ADAR, RNF220, MCM2, DTNA, PDIA6, PSMG2, URM1, SNX17, SV2A, SPG21, REEP2, HSD17B4, GSTO1, TRAPPC3, ZNF385A, SHISA5, FAM107B, MID1IP1, SERPINE2, CDK5RAP3, COMMD4, ANAPC15, CABP1, PTPRA, NPTN, RERE, VPS29, SPTBN2, MT1G, DTX3, GOLGA7, TPD52L2, EPB41L3, SPIN1, FARP1, NUP62, CNOT1, TMEM108, DUT, MAPRE2, PSD, NEBL, UBE2L3, KIF5C, TCF19, WDR54, CPSF7, GIT1, L1CAM, ITPR3, RHPN2, HMGA1, KCNAB2, HID1, SHF, CD99L2, C19ORF25, PAF1, B4GALT2, PTGES2, CAPG, SEMA4D, TTYH2, EXOC7, CNDP1, TACC2, BACE1, GABBR1, LMNB1, SLAIN1, GLG1, APBA2, CADM3, PC, PHLDB1, HHATL, SOGA1, CNTNAP2, RFC2, MAPK8IP3, CD200, REC8, TACC3, SYT13, SRP68, KLC4, CORO1C, ATPAF1, NARF, AP3B2, NUP93, FAM102A, SCN1B, NDUFS2, CBFA2T2, PSMA5, EPB41, and BCAR1.

Compositions comprising intronic domains disclosed herein include any sequence that promotes trans-splicing primarily in liver tissue. In some embodiments, the intronic domains are derived or isolated from human genes that experience liver-specific alternative splicing events. Exemplary intronic domains that promote liver-specific trans-splicing include without limitation genes such as: ORM1, ORM2, PLG, ALDOB, CYP2C8, CPS1, ADH4, PON1, CFHR3, C6, DBI, ITIH3, ITIH4, C4BPB, PTGR1, C5, LBP, PRDX1, HPD, UBB, HPR, ADH6, RPL15, CXCL2, DPYS, HAL, SLC39A14, AQP9, SERPINA10, ADH1A, HABP2, SERPINF2, ECI2, APOM, KLKB1, ACSM5, SLCO1B3, ASGR2, IL1RAP, PSMA1, GRB14, LRRC75A-AS1, BCHE, EPHX1, DMGDH, MST1, ACSM3, SEPT2, TFR2, NDUFC1, NR1H4, AFMID, HNMT, TTC39C, RDH16, GPAM, SLC13A5, SERF2, PICALM, PCSK6, TOM1L1, ANPEP, EBNA1BP2, RCL1, DEPDC7, BTG1, KRT8, ABCA6, GJB1, KMO, HSD17B13, LEAP2, PECR, LEPR, CTNND1, ENSA, MASP2, KYNU, ABCB4, DNASE1L3, ACSL5, UBE2V1, SPTBN1, RBPMS, RAB13, IL1R1, SERPINA5, FGGY, TMEM220, ACSS2, VNN1, CTAGE5, MS4A6A, MFSD2A, SKAP1, VDAC2, SLC22A7, PPP2R5C, SORBS2, CDV3, CSAD, TRPM8, TOMM5, SLCO2B1, MICU1, NCOR1, GSAP, CBWD5, ENPP1, SPATS2L, FKBP5, NR3C1, IGF2, IGF1, PLSCR4, MYLK, SLC27A5, ALDH5A1, TXLNGY, PSMB5, MOCOS, PET100, SGK1, AZ12, TJP2, FMO4, STEAP1, DMD, RNF14, RBM34, PABPC1L, ZCCHC9, ANO6, AGMAT, MASP1, IL17RB, MRPS27, MLIP, MON2, ETF1, SULT1B1, GBP7, PRKACA, PI4K2B, SP100, TMED6, NFU1, RMDN2, IP6K2, CCNB1IP1, PCBP1-AS1, SLC39A1, and PHF11

Compositions comprising intronic domains disclosed herein include any sequence that promotes trans-splicing primarily in heart tissue. In some embodiments, the intronic domains are derived or isolated from human genes that experience heart-specific alternative splicing events. Exemplary intronic domains that promote heart-specific trans-splicing include without limitation genes such as: TNNI3, TNNT2, MB, FHL2, CSRP3, FHL1, UBB, MYOM2, CD36, GSN, PDHA1, CRIP2, POPDC2, SORBS2, CPT1B, RYR2, ANXA11, ITGA7, SORBS1, NDUFC1, NEXN, MYH7B, ABLIM1, NEBL, CAST, NME2, CYB5R3, TRDN, TACC2, NDUFA5, MYL12B, FHOD3, CALCOCO2, NCAM1, GPD1L, SGCA, AQP1, AKAP1, ARL6IP4, LRRC39, TTN, TSC22D1, PPP1R12B, OBSL1, MYO15B, PKIG, HMOX2, CDK18, BTNL9, ITGB1BP2, SEPT2, CAV2, DTNA, C15orf41, ATP1A1, BCO2, MYL4, ADCY5, CLIC5, SLC8A1, RRAD, AKAP13, AK3, MYO1C, FGF12, LAMA5, ADAM15, WNK2, SUN1, KLHL7, RNF207, HIF3A, HSPG2, LPCAT4, ADH1B, ENY2, RGS3, FRMD3, CCNB1IP1, PHC2, STAB1, HAND2-AS1, PTPN3, PARVB, PKD1, SPEG, SYNE2, ZNF638, SEPT4, DAG1, SLC12A7, FBXO18, FAM107A, RBM17, MYPN, GRIP2, SCN5A, WFS1, RABGAP1L, SYNE1, HLA-DRB5, SNRPN, MEG3, RAPGEF3, SEC13, C1QTNF1, UCKL1, FRY, IP6K2, CUX1, ANK3, MYH14, ADCY4, C3orf18, MYLK3, PXN, TSPAN18, PDE1C, C6orf48, BCS1L, ATG4D, HRAS, TENM2, PLXNB1, N4BP2L2, TNXB, LTBP1, MAPK8IP3, COL1A1, GCAT, ACOT11, MTUS2, VPS45, and KCNIP2.

Compositions comprising intronic domains disclosed herein include any sequence that promotes trans-splicing primarily in muscle tissue. In some embodiments, the intronic domains are derived or isolated from human genes that experience muscle-specific alternative splicing events. Exemplary intronic domains that promote muscle-specific trans-splicing include without limitation genes such as: ALDOA, TNNT3, MYBPC1, ATP2A1, NEB, STAC3, CMYA5, MYH7, TNNI1, CA3, ASB5, PLIN2, TRDN, OBSCN, RYR1, RAP1A, SPARCL1, CSNK1A1, NPEPPS, AKR1C3, IDI2, MBNL1, FNDC5, TMOD4, ATP6V1F, DAP3, KPNB1, NOMO2, BEST3, HP1BP3, SEPT2, TIMM23, MYPN, DHRS7C, PALLD, POLR2J3, KANSL3, THOC7, MEF2C, NOMO1, YWHAZ, ZBTB16, SPAG9, CCZ1, MYLK2, ASAH1, GTF2I, CCPG1, SURF4, SMARCA2, CLTCL1, SLC6A6, CHRNB1, MAFG, COMMD6, SLC7A8, CDK11B, and DNASE1L1.

Compositions comprising intronic domains disclosed herein include any sequence that promotes trans-splicing primarily in retina tissue. In some embodiments, the intronic domains are derived or isolated from human genes that experience retina-specific alternative splicing events. Exemplary intronic domains that promote retina-specific trans-splicing include without limitation genes such as: ADGRV1, AMPH, ATP1B2, BSG, CASK, CC2D2A, CEP290, CLTC, DMD, DOC2B, EFR3A, ERP44, FARP2, FN3KRP, GRAMD1B, GSS, HCN1, IFT140, IMPDH1, INTS13, KCTD5, KIF1B, KIF1B, KMT2D, MADD, MAN2A2, MARCH1, MSI2, NAP1L4, PPP3CC, PROM1, PTPRF, PTPRF, RIMS2, SLC17A7, SLC4A7, STXBP5, TTC8, UNC13B, UNC13B, WDR47, and YKT6.

Intronic Domains that Promote Trans-Splicing Primarily in Brain Tissue

In some embodiments of the compositions of the disclosure, there may be an intronic domain that promotes trans-splicing primarily in brain tissue.

In some embodiments of the compositions of the disclosure, the intronic domain is derived or isolated from human genes that feature exons that are primarily included in brain tissue during the process of RNA splicing.

In some embodiments, the intronic domain that promotes trans-splicing primarily in the brain comprises or consists of sequences from the gene PSMA5. In some embodiments, the sequences from gene PSMA5 comprise or consist of:

(SEQ ID NO: 1)
CGAAGGGACTCGTCCGCGGCGCCGGCTCAGACCCACTGTAGCCGA
CTCATTACCGCCCAAAGTCCTGGAGCCCTCGACCGGGCATGGAGA
ACACCGTGGAGGAACCCTAAGAGAGGGAATCCTGCGAAGAGAAGT
CAGGTGGTGGCCTTGCCGGAAACGGGACCAAAGTCCTCTAGCTGG
GATTACGGGAGCCGGCGAGGGACCTATTATGTCAGGGTTCAGACA
ACCCAGCTCTGCATGCAAGCCTTTCCAATTGCCAGTCTTGGGGCG
GGCTCACATCTTCTCTACAAGGCGAACTCACTACACGCCTTGGGT
GCCTATCGTCCCTAGCAACCACCCGCGATTTAAGGCGGAAGTACC
TCCCTGGTCCTCCTCCTCCTCCGGACCAACCAAGCCCTCGTGAGG
AAGCGCGTTGGCCCAATCACCTAGGCGGGAACTACTACCAGCCAA
TAGGAAGCAGGCACAGGAGGGCGGGCTAAAGAGGTCGCGGTCGCA
CGCG

In some embodiments, the intronic domain that promotes trans-splicing primarily in the brain comprises or consists of sequences from the gene GUK1. In some embodiments, the sequences from gene GUK1 comprise or consist of:

(SEQ ID NO: 2)
TCAAGCGACCGTCCCACGTCAGCCTCCCGAGTAGCAAGGACTACA
GGCACAAAAAAGTTTTTAAATTTTTTGGTACAGAAGGGGTTTCCC
TATGATCCCCAGGCTGGTCTCCAATTCCTGGCCTCAAGCGGTCCT
CCCGGCTCGGACTCCCAAAGAGCTGGGATCACAGACGTGAACCAC
GCGCCCGGCCACATTCCCCAATTAATGGTTTTTGGAGAGTGGCCG
GTTTGGAACGGGCTAGCGGAGTCGTTCGTTAGTTAGGTCCCTGCA
GTGTAGATGGAGGACGGAAACGGCGCTGTGACATAAGCGGGAGGA
GGCGGGCTGAGGCAGCGCTGTGACGTAGCAGATGAAAGCCGGGCC
CGCGCTGCACTGAGGTACTTCTCTGAGTGACAAGCGGGACAAGTA
CGCCGTGGGCGGGGCCGGCGGGTCGTGATGTTAGTTCGCGCTGTG
TGGAGGCGGGCCGGCGTGGCGCTGTGACGCAGCGGGAGAAGACGG
GCTG

In some embodiments, the intronic domain that promotes trans-splicing primarily in the brain comprises or consists of sequences from the gene KCNAB2. In some embodiments, the sequences from gene KCNAB2 comprise or consist of:

(SEQ ID NO: 3)
GGCGCGGCCAGGCGGGGTCCGCGGACTCCGGCCTGGGAAGGATGC
GCGCGGACTAGGGCGCGGGGAGCCAAGGCCCAGAACATCGGTACC
GCGCTCCCTTCAGCCGAGGGGCTGCAGAGAGGCCAGGGGGCAGGG
GGCAGGGGGCAGGGGGCAGGGGGCAAAGGGCAAGGGCAGAAAGGA
ACCCACGTAGCCAAACGGCACCCAGCGGCCCCGACCCCAAGTGTC
CCGACGAGGGGGGACGCCTGCGACTAACGCGGGGACAGCCCCCGC
CGCGCGCCACCGCCGCACCCCAGGCACCGGGCCCCCACGCAGGGA
CGGCGTGGACCACCCTACCCCCGAACCCCGAGGCTCTGGCCAACA
CTGAGGCCCCCTGTGCCTCGAGGCCGCGTCCAACCTCCCCCGCGG
CGCCGGCCCACATGGTTCCGCCCGCCGCGGGCCGCGTTACCTAGG
AGACCGACCCGCCGCGGCCGCGCCGGAGGCCACGGAGCCCACGCT
TTTC

In some embodiments, the intronic domain that promotes trans-splicing primarily in the brain comprises or consists of sequences from the gene KCNAB2. In some embodiments, the sequences from gene KCNAB2 comprise or consist of:

(SEQ ID NO: 4)
TGAGTACCGGGGAGACCCCGGCGGGGCGCGCTTTCCACCAAGCTG
CTCCGGGACGGCGGCGGCGGGTATCCGCGAGGCCCGCGCTGCGGC
AGGCGCCGCGGTCCCGTCTCCATCTCTTCTTCCCCGGCCCTGCGT
CCCTGCCTCCGCCGCTGTCTCACCTCCTCTCCAGCCTCCTCACCT
CCTTCTCCATCGTCTCCCTTCCCCCATCCCTTTCTGTCTTTCCTT
CCCTGTCCTCCCGCCCGCCCGACCCTTCAGCTCGCATCCCTGACC
CCTGCCCCTGCCGCCGTCCCTCTTCCCGGTGCACCTCTTCCCCAT
CTCTGCCCGCTCCGGTCCTAGCAGCCTCGCCCTCCCCTTTCCTCC
ATCCTCCTCTCATCCCCTCTCCCGGCCTCTGCGCAGCCCCACCGG
GGCGCTGCTGTTCCCGGTCCTCCCCATCTCTCCTCCTGATCGCTT
CTCAAGGCTCATCCGTCGTTCTCGCTGCCTGTCCCTGTGTCGCTC
CGGT

In some embodiments, the intronic domain that promotes trans-splicing primarily in the brain comprises or consists of sequences from the gene CAMTA1. In some embodiments, the sequences from gene CAMTA1 comprise or consist of:

(SEQ ID NO: 5)
CGCCCCACCCTGTCCCCCCTGGTTGGGCTCGTACCGGCCAGGGCC
CCCTGGCCAGGAAAGGTCTGTAGCATGTATGTTTCATGTGTTTAA
TGAATTCTGTAACCATCAGCCATTCCCTGTACCCCAAAGGGTCTT
AACCTAGGCTCTAGGAATGAACTACAGAGTAACTCCAAGCCCCTA
AAATTATAGGAAAATGCATACGTAGGTACATTTCCCTGGTCCTTG
GCTGTCCTGGGTTCTCGACGCCTCTATGGCTCCCGGAGGGTAAGG
CCCCAGCTGCCCATCGAGTCTGCCGGCTGGTGTGTCCCCACGGGG
CTTCGTAGGTGCTGTCCCAGAGCCTTAGTCTTGGAGCTGGAGCCA
GACGCAGAGGTGCCCTCGGTCTCCCTGGGCCGCTGCCAGGCGGCT
GTGGCCCCTTCTTGCTCCTTCTCTGTTGCGCCGTCTTCCTTCCTT
CCTTCCTTCCCTCTTTCCAACTGAATTCTCGTTTTTCCTCTCTCC
CTAG

In some embodiments, the intronic domain that promotes trans-splicing primarily in the brain comprises or consists of sequences from the gene CAMTA1. In some embodiments, the sequences from gene CAMTA1 comprise or consist of:

(SEQ ID NO: 6)
TAGGTGGCTGGGACTGGATTCTTCTTCTGTCTCTGGTGCTCGGGA
AGGGTCTGTGGAGGGCACTGAGGGCGCGGGGCTCCGCATGCGACA
CGGCTTCGGGCTGAGAGCCCTGGTGAGCTTGCCTAGGGAGACTTC
GGCTGTTGCGGCACTGAGGCCAGCTCAGTGGCTGGGCTGGTGTTG
TCATGTGTCAGGAAGGGGAGAAGGCCTGGTGGCAGGGGTAGCTGT
CACCCACTGATGGGAAATAAACCTAGGTGCTTCCAGCTTCCAAGG
CGCCTCAGTTTTCTTTTGATGAGGACAAGAAAGAAGGCCGAGTAT
CCTGCCCTGAAATGCCTGTTGGCTGAAAGCTGTACATTCATTTTC
TCTCATGGGTTAGTTACAAGGCACATGTGGAAAACAGGCTCCCGG
AGGTTGCCAGGCACAGCTGGATTGATTGGGATAGCTGTGGGGAAA
GGAGGAAGGAGCTCCGTAATCGATTTGTGATGTCTGCTGTGGCAG
GGAT

In some embodiments, the intronic domain that promotes trans-splicing primarily in the brain comprises or consists of sequences from the gene RERE. In some embodiments, the sequences from gene RERE comprise or consist of:

(SEQ ID NO: 7)
GTCAGTATTTGAAATTTTCTCCTTGAGTGGCTATTATAAGAGGAT
CCCGTAAGAATTGATTACTGTGAAATTAGCCAACTACATAGTGTA
TTTCAAAGATAAGGTAAACTGTTGGACTTGTGATTACCTGGTTTC
TGGTATGAATTACTGAACTTATGTTTGTTTTTATGTGCATAGATA
AAGCTATATGCTTGATACTCTTTCAATTATAAACTTAATGGGTTA
TAAGAATTTATAACTTGGGCTTCATTAAGTGAAGAAGGCGCTCTT
ATACTTTGTCTTTTTGTTTTGTTTTCTTTTTTTTTTTCCTTCTTT
TGAGACAGGGTCTTGCTTTGTTGCCCAGGCTGGAGTGGTGCAATT
ATAGTTCACTGCAGCCTTGACCTCCTGGGCTCAAGCGATCCTCCC
ACCTTGGCCTTCCAAAGTATTAGGATTACAGGTGTGAACTATCAT
GCCCGGCCTAATTTATCTTTGTTAAGCTTCACCTGTTCTGTTGAT
GATA

In some embodiments, the intronic domain that promotes trans-splicing primarily in the brain comprises or consists of sequences from the gene RERE. In some embodiments, the sequences from gene RERE comprise or consist of:

(SEQ ID NO: 8)
GCCTCCTGGGTTCTAGCAATTCTCCTGCCTCGGCCTCCAAAGTAG
CTGAGATTACAGGCGCCCGCCACCATGCCCAGCTAATTTTTGTAT
TTGTAGTAGAAACGGGGTTTCACCGTGTTGGCCAGGCTGGTCTCG
AACTCCTGACCTCAAGTGATCCGCCCACCTCGGCCTCCCAAAGTG
CTGGGATTACAAGCGTGAGCCACTGCACCCGGCCTTCCTGCTTCT
TTATGTTCCGCTATCCGATCCTCACAGCTTCTTTAAACCTTCCTA
ATATTGAGAAACATACCCGATGTAAGACACTGTTAAAAAGAATCC
AGCATCTGGGGGAGGTGTTAGGGAATTGAATGTTGCTAAAGCACC
AAAGAGAGAGGAGTTTGACTATTTTGAGCTTGTCTGTGACACCTG
TTTTGTTTGGAAAATGCTAATGAAACTAGTTGGCAGAGCTGCATT
TTTGAAGTTGTTTCATTTAGTCACAGTGATAGTCATCTGTCTTCT
TTTA

In some embodiments, the intronic domain that promotes trans-splicing primarily in the brain comprises or consists of sequences from the gene ATP13A2. In some embodiments, the sequences from gene ATP13A2 comprise or consist of:

(SEQ ID NO: 9)
GTGAGGCAGCCCCATTGCCCCTTCCCAGAAGCCCCTGCTACCCTG
GCCTCCCCAACTTCCCCAACCCTCGGCCCCCCCATAAAGGCTGAC
TCTGGCGCCTCCCATCCTAGGGAGGGAAGATGGCTCTGTGAGCAC
AGGGAGGTGCCAGGGCAGCTCCCAGAGCTCGAGAGTAACCCTCAC
CTGCTTCCCCAGCCTGGAGCCGTCCCCACAGTCCCAGGCAGAGGA
TGGCCGGAGCCAGGCGGCAGTTGGGGCGGTACCAGAGGGTGCCTG
GAAGGATACGGCCCAGCTCCACAAGAGCGAGGAGGCGGTGAGTGT
CGGACAGGTGGGTTCTGAAAAAGTCCTTGCCCCTCTCTCGGCCTC
GGTTTCATGATCTGTGCAATGGGAGAGTCAGACTTCATGTTCTCC
CACCACCTTTTAGTTCTGTTGTCCCAAACATCCAGGATGCTGTCT
GAGCACGTTTACACTTTCAGGCCTCTCCCCACCTCTCTGTCTGGT
GACA

In some embodiments, the intronic domain that promotes trans-splicing primarily in the brain comprises or consists of sequences from the gene ATP13A2. In some embodiments, the sequences from gene ATP13A2 comprise or consist of:

(SEQ ID NO: 10)
CTCGCTGCGTTCGCAGAGGCTCAGCGGCTACTGTGGCAGTCCATG
GAGGGTCATCGGCTATCACGTCGTGGTCTGGATGATGGCTGGGAT
CCCTTTGCTGCTCTTCCGTTGGAAGCCCCTGTGGGGGGTGCGGCT
GCGGCTCCGGCCCTGCAACCTGGCCCACGCCGAAACACTCGTTAT
CGAAATAAGAGACAAAGAGGTGAGAGTCAGGCTGAGCCTGGGGCT
AGAGAAGCGCAGTGAGGGTCAGGGTCGGGGGTGGAGGATGCTTGG
GTGCCAACCCGGCCACTGACTTTCCATGGGACATTAGGCAGGTCC
CGCTCTTCTCTGGACCTCAGCTTTCCCATCTGAGCATTGGGCAGA
AGGGACGGGTCGGGTGGTTTCTGGAGGGCTCTCCATAGCCTTGAT
TTCTGCCTTCTCCAGCAGGGGCCGCCTGGCTGTAGGACAGGGCCT
GGGAGGGGAGGGGACTCTTCTGAGACTGAGTCCATACCTCTGTGT
TCCA

In some embodiments, the intronic domain that promotes trans-splicing primarily in the brain comprises or consists of sequences from the gene CAPZB. In some embodiments, the sequences from gene CAPZB comprise or consist of:

(SEQ ID NO: 11)
GTGAGCAGAACGCCTAGGGAGAGTGCTGGCTCCGCTCGTGGTGTT
TGTTTTGGTAAAGAAGGCAGATGCTATCCAGACTTTTGAGGAACC
TGGGGTTTCACATAGGCCCCCACAGATAGACCTGCCCTCCTGCCA
CTTGACCCTTCTGCAGGACAGGCCCAGCACTTAGGTTGGAAGGGC
CAGAGAACTTCTTCCTGACAGGATCAAGGGATTTTTGGAGCATGT
TTGTATGTGGACTTTGGGCCTCAAAGTGTTTTGAGGACCATGGTT
CCTTGTTTAGTCAAGGGATGTTGCTGCTGAACGAGGGTGAGTAGT
TGAGATGGGTTAAGCTTTCTTTTGATGCCTTCCTGTATGCTGATT
AAAAACATGGGCAATGCCACTTTTAAGGCCTTCGGATTTGTCCAG
TAACTTTGGCTGAAAACTCCTTGTTTGGGGAGGTGGGCCTGGCCC
CAGGCCTTGATGGAGAACGACTTCTCCAGGGCAGTGGTGGGTGAG
GACT

In some embodiments, the intronic domain that promotes trans-splicing primarily in the brain comprises or consists of sequences from the gene CAPZB. In some embodiments, the sequences from gene CAPZB comprise or consist of:

(SEQ ID NO: 12)
AGCTGGAGTCCCACTCACTACCTCTGTGTTCAGGCTGGTCTCTCC
CATAGCCCAGGCTAGACCCTCCCTCCCGAACCGTGTCCCCAGCCA
GGCACCCCCTCGGTGACCCTGGCGTGGGCCCCCTTCCTCCCCCGG
TCTAGGGCTAGGTCTGCTTTCCCCCGTCTCTCCGGCCCAGGCTAG
GCCGTCGTTTCTCCCTGCCATGGCCCAGGCTAGGCCGCTGCCTCC
CTCTGTCCCTCCCCCTTTGCCTTCTCCATGGCCTCAGGCTGGACC
CCGGTCAGCCCCTTCTGCAGCGGCCTAGGTTTGCTTCCCCTCTTC
TCCGTGGCCTCAGCCTGAATCTCCTCTTCTCTCTGTGAACTTAAA
TCTGGACCTCCTCTTCTCCGCCCATGGCCCCCGTCTGGACCTCAC
TGCCCCCTCCCCACCATGATCCCCTTCCCTGGACTTTTCGCCACT
CCTAGCGTAACCTGCGTCAGCCCCGCTCGGTCCCCGCCCTGAGCC
TCTC

In some embodiments, the intronic domain that promotes trans-splicing primarily in the brain comprises or consists of sequences from the gene KDM1A. In some embodiments, the sequences from gene KDM1A comprise or consist of:

(SEQ ID NO: 13)
CTAGGTGGAAATGGATGGGGCCTGGTGGCTGACAATCTCACATAA
TCTCATACAACTGAGCCTCGGGTTAGAATGGTGTACCGGAGATGC
AGTCCTGTCATCTGGTGTCAGTCAAATGTGGCAGCAATCTTTTGG
TTTGGTTTTTGTTTGTTTGATTTTGTTGTTGTTGTTGTTGTTGTT
GTTACTGTTCTGTGTTTTTCCTTTCCTCTTTTGAGTTTTGCTTTA
TTCCTCACTGCTTTCTGACACTTCCCCCTCACTCCCCTGTCTTTT
GTTAACTGCCTTTCTTTTGGCCATTTCTGTGCCTTGACTTTCCCA
TTTGGTCTCTGTACAATCTGTTCCTTATTTCTCTGGTGGTTGCAG
CTTCTGAGCTAACAGAATGCTTCTGTCATGCTATGTGTTTAAAGC
CTTGATTTTATTGCTGTCATACAAAACTAAACTTGATGTGGCTGT
TGGGCTGTTATTTACAGTTTATTTCTCTCTCTGCTGCACTGGTTA
AAAG

In some embodiments, the intronic domain that promotes trans-splicing primarily in the brain comprises or consists of sequences from the gene KDM1A. In some embodiments, the sequences from gene KDM1A comprise or consist of:

(SEQ ID NO: 14)
TATTTTTTTCATAATTTTTCAAATCATTTTCCTCACTGTTTACAA
TAACCCATTAAAAGCTTGAAACCTAAATCTTTTCCTTTCAAAGAT
CCTCTCCATCATACATTCACACTGGTTTGTTGTTGTAGAGTTGCG
CCTATATGTTGGGCTGGTGCCATTTGAGTTTTTGTCATTGAAAGC
ACGTGTAAAACATTAGGTTTTTTGTGGGTGGTGTGGGTGGACTAA
GGGGGAAAGTGTAATGAATCCAGTGGGCTGCTCAGCAAACAGAGG
GAAGAAACAGGTCAAATTTGGTCCCTCTAACCTTGACAGTGTCCC
TTTAAATATTTCTCTACCACACACCCTGGACCCCAGTTACTGAGC
CTGAGTGCTGTGTACTTTTTCAGTATGAACTTCCCTTTGTGATTG
TCATCCTAGTTGCAAAGATTAAAGCTTGTCTGAAGCAAGCCATCC
AGTTGACACTGGAAGAGCTTTAATCTGTACCATTTGCACACTTGT
GTTT

In some embodiments, the intronic domain that promotes trans-splicing primarily in the brain comprises or consists of sequences from the gene STMN1. In some embodiments, the sequences from gene STMN1 comprise or consist of:

(SEQ ID NO: 15)
GTTTGAAGATGTCAGTTGCCATGTCAATTTTTATATTATTATTTT
TTCCATTTCCTGGTAATGAGATGCTATGTTATCTTTATTTATTTA
TTTATTTATTTGTTAGAGACGGGGTCTCGCTTCGTCGCCCAGGCT
GCAGGGTAGTGGCGCAACTATAAGCTCACTGCTGTCTGGAATTCC
TGGGTTCAAGTGAACCTCCCACCTCAGCCTCAGCAGCTGGGACTA
AAGGCACATAATACCACACCCAGCTAATATTTAAATGTTTTGTAG
AGACTGTGTCTCACCATGTTGCCCAGGCTATTCTCCAACTCCTGG
CTGAAGCAATCCTCTGGCCTCAGCCTCCCATATTCTTTTTTAAAC
AGCTTCTTTAGAGCACAATTTACATACCATAAAGTTGACCTCTTT
TAAAAGTGTACATTGCGCCGGGCGCGGTGGCTCACACCTGTAATC
CCAGCACTTTGGGAGGCCAAGGAGGGTGGATCACGAGGTCAGGAG
ATCG

In some embodiments, the intronic domain that promotes trans-splicing primarily in the brain comprises or consists of sequences from the gene STMN1. In some embodiments, the sequences from gene STMN1 comprise or consist of:

(SEQ ID NO: 16)
GAAGCCATTTTTCCAAGGTCACATAGAAAGCTAATGATGGAGAAT
AGACTCAAACTTGCTGCACTGCCTGAGTTTTGGATTTTCTTTTTT
TCCTTCCATTCCTTCCCTCTGCCCTTCCCTCCCACTGTCCTCCAA
TTCATATTTGTCACATGCCTACTTTGCACCAGCCCAGTACCCTGA
CCTCCTTGCCTTAGAACCTTAGGCTTCCAGAGCCTAGGTCATCAC
CACCTCTTCCTATCAAACCTCCTCCCCACACCCCGTTCCTGCCTG
ACCTTTCCTACTCGTTAAAAATCCAGATGCCTAGGTTGCTTCCCA
TACCAACAAGATCAGAATGTCTGGGGGTGGGGGCCAGGTGTCAGT
ATTTTTTAAAGATCCCTGGATGATTCCATTTGTAGCAAAGTTTGA
GAACCACTGTTTTAGCCCTGATGAAGGGCCCCTCCACCTCCTGTC
CCTTGGCCTCTGGGTTGCAGATGACCTGGTGATTTTTCTTTTTCT
TCCA

In some embodiments, the intronic domain that promotes trans-splicing primarily in the brain comprises or consists of sequences from the gene EPB41. In some embodiments, the sequences from gene EPB41 comprise or consist of:

(SEQ ID NO: 17)
AACGCCTACTGTGTACCAGACCTATGCTTGACATTTAACTTACAT
CGCTAATTCTCAGGAAAACATTGTAAGATTTATGGTTGACGAAAT
TCAGATCCAGAGAAGTGACTTGTTCAGGGTCACGCCGGACGAAGT
GGTAGGACAGGCGATCCAGGTGAGTCTCCTCAGGGTCACTTCCCT
AGATCCCCTCTGACCAACCCCTCTACCTAGTAACCTTAGGAGCAG
TACCACGTGTCAAATTGCTTCACACTTTTGTCATATGACGTCACA
CCAGTCAATCCAGGAACTCCCTGCCCTTCCCTCCCCTGACCTCTG
GGGTCTATCAAAGCAGCCCCCGGCTCAGTGTCCCCGCCGGACGAG
GCTACACCTGCAGCCCCCGCCCCTTCCCAACTGCGACTCTCGCAC
TCCTCTGCCCGGGCCTCCCCCTCCCCGCCCCGCTGATCGGCAGTT
GCCTGCGCCAATCAGAGGGCCTAGCGGGCGGGGCGGGGCCTCACG
ATGG

In some embodiments, the intronic domain that promotes trans-splicing primarily in the brain comprises or consists of sequences from the gene EPB41. In some embodiments, the sequences from gene EPB41 comprise or consist of:

(SEQ ID NO: 18)
TGAGCCTGGACCACCTGGGGGGCGACCCTCGGTCCCCGGGAGGGA
CGGGGTTAGGGACAGAAGAACCTACCCCCAAGGGCTCGGACCGTC
CCGGGAGAGGCGAGACCAGGGTCGAAGGGTCCAGGGCTGAGGGGT
CCAGCGGTCCCGAATTCCAGAATCCGAACTTGGGGTCCAAAGGAG
TCTGGGCATCTTAAAGTTCAGGGTGCAGGGAGGGGCGTGGGAGTC
TGGAGAGGGGGTCCGGGAGCTCGGATCCGGAGCTAGACACGTCCG
GGTCCGGCCGGTCCTGGCTGTCTGGGGCGGGGGTCCTGCATTCGG
TGTCCGCGGGAGAGTCCCTGCAGGTCGGCGCAGCCCCCGGCCGCC
CCCTAGCCCCGCCTTGCCCGGCCCCGCATGCTCCTGCCGGGCCCG
CAGCAGCGCTGGAGCGGGCTGGCGGCTAGCAGCGGGAGGGGGCTC
CGGGGCCTGGAGCCCCGCGCCCCGCTCCGGCCCTTACGTAACTGA
CTTT

In some embodiments, the intronic domain that promotes trans-splicing primarily in the brain comprises or consists of sequences from the gene TRAPPC3. In some embodiments, the sequences from gene TRAPPC3 comprise or consist of:

(SEQ ID NO: 19)
GGGCTTTAACATTGGAGTCCGGCTGATTGAAGATTTCTTGGCTCG
GTCAAATGTTGGGAGGTGCCATGACTTTCGGGAAACTGCGGATGT
CATTGCCAAGGTCATTATTCTGGGCCACCTGTCCATGCTGAAGAA
TTGCTGCTGAGAGGCACTGCTTACCACTCTTTCTATCTCCCTTAG
AGAGGCCTTTAATCTCCCAAAAGACTGGCTAGGCAGCTCTTTGGG
TCAAAAAAACATTCCAGGTCGTTAGGGAATAGCATAGATTATTTA
ATCCCTGTGGATTAAAGCCCCACTGAAATCAAAGCCTGAGCTTTT
CCACTGTGAAGGTGTAGCAGAGTAGCATGGGGGTAAAGAATACAG
ACTCGGTCGGGCGTGGTGGCTCACATCTGTAATCCTAGCACTTTG
GGAGGCCCAGGCGGGTGGAACACAAGATCAGGAGTTCAAGACCAG
CCTGGCCAAGATGGTGAAACCCCATCTCTACTAAAAATACAAAAG
TTTG

In some embodiments, the intronic domain that promotes trans-splicing primarily in the brain comprises or consists of sequences from the gene TRAPPC3. In some embodiments, the sequences from gene TRAPPC3 comprise or consist of:

(SEQ ID NO: 20)
CCCTGCTGCTTTGACCTTGCAGGCCAAATTTCCTGGCTCCTCCAA
AGGGGATGATGTTCTTTCCCTCCAGACACAGTTAGAGGGAATCCT
GACAGTTGCATCAAGGCTGGTCCAGACTCTCACCACGCTTTCTCT
TTTGTGCTGCTTGGTCCTGACTGCCTTTTGAATCTCTTTTAGAGC
TCTGAGCTCTTCACCCTGACCTATGGTGCCCTGGTCACCCAGCTA
TGTAAGGACTATGAAAATGATGAAGATGTGAATAAACAGCTGGAC
AAAATGTGAGTGAGCTCCTTCATAGGAGAGACAGAAATGGGGCCT
CCCAGCACACTGAGTCTACATTGTCCTTTAGAGCTTAAAACAGGG
TTCTCTCTCTTTCTTAGCCAACATTTTGGGTACATTTCTCACTAA
ACTCATTTACTGGGATGTTTTTGAGATTATCCCCATTTTCCCTTT
ACCACCAACAACCTTATGTTCAAACATAAGACATTTAGACTCCAT
CATA

In some embodiments, the intronic domain that promotes trans-splicing primarily in the brain comprises or consists of sequences from the gene CDC20. In some embodiments, the sequences from gene CDC20 comprise or consist of:

(SEQ ID NO: 21)
GTGGCACTGGACAACAGTGTGTACCTGTGGAGTGCAAGCTCTGGT
GACATCCTGCAGCTTTTGCAAATGGAGCAGCCTGGGGAATATATA
TCCTCTGTGGCCTGGATCAAAGAGGGCAACTACTTGGCTGTGGGC
ACCAGCAGTGCTGAGGTGCAGGTGAGACGTGTCCAGTGCTGTCAT
TCTCACCAGTCCCAATGGGCTTGCACAGCTGGAGGATATTAATGC
CAAGTCCCAATCTCTGATCCTAGTGGGCTTCTCTCTCTAGCTATG
GGATGTGCAGCAGCAGAAACGGCTTCGAAATATGACCAGTCACTC
TGCCCGAGTGGGCTCCCTAAGCTGGAACAGCTATATCCTGTCCAG
GTCAGTGGTTTTTGTTGGTCTATGGTAGTCTGATATTTGCCCACC
CTCCCCTTGACTGTACACCCCTGAACTGAACCAGCTCTGGCTTGC
TTGCATTTGGTGCTGCCACAGAACCTGATTCCCTTCTTTCCTCCT
CCAG

In some embodiments, the intronic domain that promotes trans-splicing primarily in the brain comprises or consists of sequences from the gene CDC20. In some embodiments, the sequences from gene CDC20 comprise or consist of:

(SEQ ID NO: 22)
TGAGTAGGGTTGGGCTGAAGCCTCTGCAGACCCTCACCTATAATC
TGGGGACTGTTAAGGGGAGAAGGGATGGTAGGATTGGACTGGGTT
AATCTGTGACCCCTAGAGCTCCTTGTCTGGCTCTAGTACCTGCTG
ACCCCACCTTTATTCCATCTAGGCCGTAGCATGGTGTCCCTGGCA
GTCCAATGTCCTGGCAACAGGAGGGGGCACCAGTGATCGACACAT
TCGCATCTGGAATGTGTGCTCTGGGGCCTGTCTGAGTGCCGTGGA
TGCCCATTCCCAGGTAATCTTTTGCCTGTTCCTGCCTCTCCCACA
TGCATTCTTACCGGGCAACTACATTCACTCCAATGGCTTCACCAA
CTCTATGCCCTGGTGATTCCCAAACTTCTAACTCAGCTCTCACTC
ATGAGCTTCATTTCCAACAGTCTGTGGCTATCTCCAATCCTATGT
CCTATAGGCACCACCACCTTGATCTGTCCCAAATTAACCTTGTCA
CTTC

In some embodiments, the intronic domain that promotes trans-splicing primarily in the brain comprises or consists of sequences from the gene B4GALT2. In some embodiments, the sequences from gene B4GALT2 comprise or consist of:

(SEQ ID NO: 23)
GGATGTATGGCTGTGGAAGTCCAGGAGCAGTGGCCTTGTTTGTGA
GTGTGGGACTTAGTGTGTTACTGTCACCCGGGTGTGTCTGTGACT
GTAGTTCTCTGTGTGTCTGAGTGTGGGTGACCAGCTGGGACCAGG
GTGTCCCTGTGATTCTGTGTGCCTGTGAGACTCACCCTGCTCCCT
GAAGCACTGCCAGAAGTGGCTGCCGCTCTCTTGGAACCACAGCTG
GAGCCATCGCCCAGGCAAGACCCCCTTCCTGCCATGCCCAGTGCT
GTGCCCATCTCCAGGCCCAAGCATCCCGTTCCGGGAATCAGAACC
AGCTGTGCTGGGCAGGACCCAGAGTCTTGGTCCCAGTCCCAAGGG
GCTGGCTTTGAGGTCTTAAGTGAGGGGTGTCCCCTGCCTTGCCCC
GTATTTTGACGTTTTGCTCCAGTGGGAAGGGATTCTGTAAACATC
CAGTCTGTATTTGGGCCCCCCGGATGCTGAGGGCAACTTCCCAGC
CTGG

In some embodiments, the intronic domain that promotes trans-splicing primarily in the brain comprises or consists of sequences from the gene B4GALT2. In some embodiments, the sequences from gene B4GALT2 comprise or consist of:

(SEQ ID NO: 24)
TAGGTGCACAGTATTCCGTCTCTTCCCTCTTCCCCAAGCCATGAG
TTCTGGGTTAAGGATGCTGCCATGTGAGGTGTCTGTTCTCAGGGG
CACAGGGGCACATGGGGGTCTATAGGTAAACATGTCTCCATGCTT
AGGTACAAACCAGTGTGGTTAGAGTTCACGTGTCTGTGTTCCTGG
GAATGCGTGAGTGTGTGTGTAGAGTTGTGCAATAAGGGTGAGTGC
CTGAGTGTGTGTGTCAGTGAGGTGGAGGAAGGTAAATGTATGTGG
CTTAGTAGTGGGGTGTGCTTGAGGATGTGAGTACTGAGTTGCCGG
GAACTGTGCCTGGGTGTCTGTTTGGAGGTAAATTCTTGAGTCTGT
GAGTGTGAAAGGGTATGAGCAGGTCAGTGTGAGGTGTGGCCCACG
AGTGTGAGCAGCTGAGTGGGAGGTAGGTGGGCAGCCTTGCCTGTC
CTGCCCTGACCTGCTGGATCTGTTTCCCTCCCACCCTGCTCAGGC
CAGC

In some embodiments, the intronic domain that promotes trans-splicing primarily in the brain comprises or consists of sequences from the gene RNF220. In some embodiments, the sequences from gene RNF220 comprise or consist of:

(SEQ ID NO: 25)
GAGAAGCATTCCAGGAACAAAGCAGCAGGTAAGATTTTTCTGGGA
AAAGACAGAGATTTTGAGGGACCCAGGATTTGGGGGTCATTTAAG
CAGCAGTGGATGTCAGTCTTCAAGCTTTGCCCATTTACTCTTGGT
CCCTTTTGGTTCAGGAAAGGAAGAACAACAAGCACGAAACCATTC
ACTGAGACCTTTGCGGGTGGGGGAGGAGGGATGTAAACAAAAGCC
AACGCTCTCTTTAGTTCACAATGGCTCGCGACCCCAAGACTTCCA
AGCTCTTTGGTGAGGGCCAGCAGGTCTGTTCTCCAGGAGTGCCCT
GGGTCTGGACTTCATTTCCCAGAGTCCCTTAGTGCCCTAATGTAT
TCAGTGCTCAGAGTGCCTGTATGTGTGTGAGTGTGAGAGAATGTG
TGCGTGCGTGTGTGTGTGCGCGCGTGTGTGTGTCTCCGAGTCCCT
GGGAGAGTGGAGGCTCATTCACTGATTAGATCCAGCGCTGAGAGG
CAGC

In some embodiments, the intronic domain that promotes trans-splicing primarily in the brain comprises or consists of sequences from the gene RNF220. In some embodiments, the sequences from gene RNF220 comprise or consist of:

(SEQ ID NO: 26)
TATGGAGATCAGAAAGGGGTGTGGACGTGTGTGCGTACCCAAGAG
GGGTGGGTGCGAGGGCGGCCGGCTTGTTCAGGAACCTGGCTAACT
TTCCGGGTCCGTTTCCTAGGGATTCGGGCGGTTCCCTCTGGACGG
GGGCAGGGACTGGGACTCGGACTGGCGCGTCTTGGATCATCGCGA
CGGAGCCTCTCGGCATCGGCGCCCTGCGCGCTCCGTGCGCTGCCT
CGCTCAAACTTTCCGTCTTGAGTTTTCCTTAATGTGTTTTATTTG
GGGAGGGGGGGAGGTGCAAGGGGGAGGGACCAGACGCGGGAATTG
GACAAGTTGGGAGGGTGAGGGCACTGCAAGGGAAGGGAGCAGGAC
TTCTTTTCTCCGTCTCTTAATAAGCCCCATTCCCACTTTGTTCGG
GTCGATATCGAGAATGCCTTCTCGCCGGGCTCTCTGGTGCACCCC
CCGGTGATTTCCGGGCTCCCCTATGTACCCCGCGTTGATTTCCGG
CGAG

In some embodiments, the intronic domain that promotes trans-splicing primarily in the brain comprises or consists of sequences from the gene PRDX1. In some embodiments, the sequences from gene PRDX1 comprise or consist of:

(SEQ ID NO: 27)
GCAAGCGTGGACTCGAGTCGAGTCTTCCCCCGGCAACTCGAGCAC
GTGGCGCGGCGTCTGGGCGGGCTGGTGGTGGGGGTCTCCGAGACT
AGCCTCCTGCCGGACGGGGTGGGTTCGGGAAGCTTCTGGAATGGA
CCTGTATCCAGACTCGTGGCTGTAGAAGACAGCCTCTATGTATTT
GGCTGTTCCGGGTCCTGAGCCTTGACTAGCTGCGGAGCTTCGGGT
ACCCTTTCCCCGTCTTTATTTTCCTGTACGTGGAATGGAGTTAGC
GATGAGCCCAAATGTCCCCGCTGAAGCACTCTCTCTGCTGGTGCT
TTCTCCAGTGCTGGGGAGGGACAGGGGGCACCTGAAGTGGGGTTT
AGGGATGTGGCCCTCTACGCTGGGCAGATCCCCTGAAGGGGCCCG
GTGGCGGCCTGAATCCCAGCAGCCTTTACCCCGAATTGGTTGGGT
TTTGTACTTGGGGGCCACTTGGGAAAGAAGGATTGGACGAGAAGG
CCAT

In some embodiments, the intronic domain that promotes trans-splicing primarily in the brain comprises or consists of sequences from the gene PRDX1. In some embodiments, the sequences from gene PRDX1 comprise or consist of:

(SEQ ID NO: 28)
AAGCGTTCGGGTTCGGGAGCGGCCACAAGGGCGCCTCACTCCAAC
CTCAGCCATCCGCAACAGGGTGGGGTATTAGTATTTTTCCGCACC
CGCCCCAACGAGGGAAAGCGCCGAGTCATTCCGGCGGGGCGCGCC
GGGCGGGGCTCGGGCCACATCCCCCTCCTCTCTCTCCTCCTCCGG
CACCCCCGTCCGTTCCGACTCTCGCGAGATCCCTACTGGCTATAA
AGGCAGCGCCCCGGAGAGCTCTTGCGCGTCTTGTTCTTGCCTGGT
GTCGGTGGTTAGTTTCTGCGACTTGTGTTGGGACTGGTGAGTGTG
GGCAGTGCGGCCCCTGCGGAGTGAGGCGCGGCGCGCCCTTCTTGC
CTGTTGCCTCTTCCTCCTCCTGTCCGGGGCCCGCCCGCGCTCGGG
TGGGGGTGCTGTGATGCGTGAGGCAGCCGGGGGAGGCCCGGAGTC
CGAGACTGCTTGAGCGCTGCGCACACCCCTCTCGTGGGCCCCCCA
CGTA

In some embodiments, the intronic domain that promotes trans-splicing primarily in the brain comprises or consists of sequences from the gene ATPAF1. In some embodiments, the sequences from gene ATPAF1 comprise or consist of:

(SEQ ID NO: 29)
GTAGTTACAGTAGAAAATTGGGATTCTACATTGTGTTTTAAAGTC
AAATTAGAGGTGTTTATTTTACTTGGAAAATACTTTTTTTGCCTT
GGGTATCCTGAATTTTAAGTGAATCTGCTGAGTCTTAATGACTAC
TACTCATCTGACAAAGGGCTAATATCCAGAATCTACAGTGAACTC
AAACAAATTTACAAGAAAAAAAAAACCCCATCAAAAAGTGGACAA
AGGATACGAACAGACACTTCTCAAAAGAAGACATTTATGCAGCCA
AAAGACACATGAAAAAATGCTCATCATCACTGGCCATTGGAGAAA
TGCAAATCAAAACCACAATGAGATACCATCTCACACCAGTTAGAA
TGGCAATCATTAAAAAGTCAGGAAACAACAGGTGCTGGAGAGGAT
GTGGAGAAATAGGAACACTTTTACACTGTTGGTGGGACTGTAAAC
TAGTTCAAACATTGTGGAAGTCAGTGTGGCGATTCCTTAGGGATC
TAGA

In some embodiments, the intronic domain that promotes trans-splicing primarily in the brain comprises or consists of sequences from the gene ATPAF1. In some embodiments, the sequences from gene ATPAF1 comprise or consist of:

(SEQ ID NO: 30)
TGAATCCTAACCCTTCCACTTACTAGCTGGGTGACTCTGAGCAAA
TTGTTACTTCTCCATGCCTCAGTTTGCTTATCTGTAAAACAGGAA
TAATAACAGTACCTACTTCACAGAGTTGTGCTGAAGAGATAATAC
CATTTAAAACTCTTTAGCGCCGTCTCTGGCATAAGTACTTAATAT
ATATTAGCCATTATTATCCTTAAATAGGAATCAAAGCTGAGGAAA
AGCATGTTGAAAACTGTGTCTGGGAAGATTATTCTAATGCAGTGT
TTTTCAAACTTTTTGACCATGACCACAATAAGAAATATATTTTAC
ATTGCAATTCAGTACACATGTAGGTATATGATTGTAACTGAAAAA
AAAATGTATAAAATAATGCTAACTTTTACAACATGCAGTCCATTC
TTATTACTTTTTATTCTCTTTCATCTTTTAAAAAACTCTGATCAC
AATCCACTAAATGTATTTCACAACCTATTATTTGAAAAATTCTAA
CCTA

In some embodiments, the intronic domain that promotes trans-splicing primarily in the brain comprises or consists of sequences from the gene PSMA5. In some embodiments, the sequences from gene PSMA5 comprise or consist of:

(SEQ ID NO: 31)
GTGAGTTGGCTGCCGGTGAGTTGGGTGCCGGTGGAGTCGTGTTGG
TCCTCAGAATCCCCGCGTAGCCGCTGCCTCCTCCTACCCTCGCCA
TGTTTCTTACCCGGTCTGAGTACGACAGGTCCGTGCAGCTGGGGG
ACGTCGGGTGGGAATTATGGGTGGGCAGGCCCGGGACCTGGCCGC
GCGGCCTGGGTTCTGCCGTGTCATGGGGCTCCCCAGGAACCCGAG
ACTGGCCGGACCTGGGGATGTTATGCCCTCCTCAGGCGCCCGCCG
TCCTTCCCCAAGCTAGACCACATCGGCGGCACTTGTCCTACCGCT
GAGTCACCCCTGGGTCCTAACTCGGTGCGGAAGCGAGAAGCTCCG
GCTGGTGCTTGGTAAAGAAGGCGTGAGGAGCCGGAACTCCTACCT
TGAACCCGGAGAAGGCCAAGGGTCACTCTTTGCTCCGTTCATCTT
TTCGTGCCTAGTATGACTGCCTTGAACAAACAAACAAAAAGTTAA
ACGT

In some embodiments, the intronic domain that promotes trans-splicing primarily in the brain comprises or consists of sequences from the gene AMPD2. In some embodiments, the sequences from gene AMPD2 comprise or consist of:

(SEQ ID NO: 32)
GGAGCAGAGCCAGAGTGTGCAGAGACAGAGGAAAAATCCAAAGAG
GCTAGGGTCTCTGTCCCAGACCCCCAGACAAGGGGGTCTCCTGGC
CTTCCTGCCTTCCTGGGAGGTAGCTATTTGGCGACGGTGGAATTT
TCCCGGGACAGGCCCCTCCCTCCTGGGCTGGGATTCTCGGTTCTT
CCCCCTCCTCCCAGAGCTGGGCTACCCCGGGAGCTGAGGGTCAGA
TGTGGAAGGAAAGTGGGTGCTGGGTTTTCACGGAGTATGCCTGCC
CTGCCCCGGGAGGACTGTGTTTGAGCTTGGACTCGCTCGCTGCTG
AATTCCTGTTTGTTATTCTTTTTTCTTCCAGGCTAGCTGGAAGGA
CCAGCTAGCCTGGACTTTGGCTTCTTTGCCCGGGAGCCCTGGCAT
GATGGGGTGAGGGCTCTTGGTGGGTTAGTGGAGAGCTGGGGGCTG
GCCCTTCCCCATCTTCTTTTCTACCCACCCCCTCCCCGCCCCCCG
CCAG

In some embodiments, the intronic domain that promotes trans-splicing primarily in the brain comprises or consists of sequences from the gene AMPD2. In some embodiments, the sequences from gene AMPD2 comprise or consist of:

(SEQ ID NO: 33)
TGAGTGTGTGCTTCCTGGCCTCAGGATAGATGCTGGGCTCTGTCT
TGCCACCTCTCGCCCAAGGTGCTGGTCCCCTCAGAGGGGGCCACC
TCCTCCGGCATCCTCTCTGTGGTGGTGCCCCTCAACCTCAGTCCC
AGGGACAGCCGGCTTGGAAGGTGGGGTGAGGGGTGGTGGGGGGCG
GGGGGTGGATCTGAGATATCTGTGCCAGGGATGCTGTCCTCTGTC
ACAAGGAGTGGGCATGGTGTGGGTGCAGCAAGAGGTGTGTATGCA
CACGTGTCAGCGTGTGCATGTGCTAAGACTGCGTGTGAAGCATGT
GTGAATGGAGCGGGGGTGTGTGGGACTGACTGTTTTGTGGACTGG
CCACTCTGACTGGCGCCTGTGCCTTTACCAGGCAGGCTTAGGCTG
TTCTGTGGCTAGTTCCACTTTTTAGGACCCCACATCTAGATCCCC
CTCACTGGGGGTCTGACATCTGGGCACAACCATTGCTGGCTGGCA
TCCC

In some embodiments, the intronic domain that promotes trans-splicing primarily in the brain comprises or consists of sequences from the gene AHCYL1. In some embodiments, the sequences from gene AHCYL1 comprise or consist of:

(SEQ ID NO: 34)
GTGACGTCACGACAGAGTGCCCTTTCTCCCCGCCTCTTCCCCCTC
CCGGGAGCTGCCAGTACTTGACGTGGCGTCACCGCCCTCTACCCT
CGCTTTGCGTGCGTGTTTGCGTACAGCGGAGGTGGCGGCGCGGGC
AGGTCGGAGCTCGGAGCTGCTGTTCTGGTTCTCTTGTGGCCGCCG
TCGCTGTCCGGCTGCCTTGGGCTGCCGAACAGACAAGGCGTGGGC
CACAGCACCTCAGAAGCCGACGCAGCTCGACGCAGGGGCCGGCAG
GAGGGTGGGCGATCGCGTGTCGGAGGGCGCCGCGCGGGCAGGCGG
GCGGGCGCCAGAGGGGGAAAGAGGCGGGGGCGGCGGGTCAGCCGC
TGGCCGGGCCGGCCGGGGAATGTCGATGCCTGACGCGATGCCGCT
GCCCGGGGTCGGGGAGGAGCTGAAGCAGGCCAAGGAGATCGAGGA
CGCCGAGAAGTACTCCTTCATGGCCACCGTCACCAAGGCGCCCAA
GAAG

In some embodiments, the intronic domain that promotes trans-splicing primarily in the brain comprises or consists of sequences from the gene AHCYL1. In some embodiments, the sequences from gene AHCYL1 comprise or consist of:

(SEQ ID NO: 35)
TGAGTGGGTAAATCTGGCAGAACTTCCATCAAATCTTTGGGAGCA
GTGGCCGCTTTATCCAACAACTGCGTGATAGCTGGGAGGCAAGAG
AAAAGAATGCGTGAAGCTAGCTCTGATATACAGGTGAAAGCGATG
AGGGTAACATGAAACTGAAAATCTATGATTTTCACTTTCTACAGC
ATTCCAATTACTTCGAAATATGTAAGAAATTGACATTTGGAGGTT
GGTGACAGCAGACACTGTCATGGATACAGGTGGATCCTAGAGTCA
TGTGAATAGATCACTTAGGCATCCCTGGAGAAAGGAGCTTGGGTG
GGTTGGAGTAGTGGGTGGTAAGGGAAGATTGCTCACTCTTGACTG
TACTTTTGAGGCAGAACAAGTTTGATCTATTGTGCTGTAGGTTGA
CTCAAAGGACACCCTTCGAATTCTCTCATAAATGGCAACCTTTGG
AGATTTTAGTCTGACTAGCTCTCCGAGTGGTGGAATTGGGATCCT
TATT

In some embodiments, the intronic domain that promotes trans-splicing primarily in the brain comprises or consists of sequences from the gene SV2A. In some embodiments, the sequences from gene SV2A comprise or consist of:

(SEQ ID NO: 36)
GTAGTTGGAATGTGTTGGAGGCAGGGCAGTGGATTCAGGGGAGGG
GTGGGGTGAGGAAGGAGGGCTGAATGGGAAGAGCATCCTTAGGGG
TGAGAGGATATACATTTCCATTTTCTCCTCTAAAATCTGGTAGCT
TTGGCCGGGCACGGTGACTCATGCCTGTAATCCCAGTGCTTTGGG
AGGCCGAGGCAGGCAGATCACCTGAGGTCAGGAGTTTGAGACCAG
CCTGGCCAACATGGTGAAACCCTGTCTGTACTAAAAATACAAAAA
GTTAGCTGGGTGCCTGTAATCGCAGCTACTCAGGAGGCTAAGGCA
GGAGAATTGCTTGAACCCGGGAGGTGGAGCTTTCAGTGAGCCGAG
ATCGCACCACTGCACTCCAGCCTGGGCAACAGAGCAAGACTCAGT
CTTAACTGTTAACCTCAATTTTTGGTCTCCTTTC

In some embodiments, the intronic domain that promotes trans-splicing primarily in the brain comprises or consists of sequences from the gene SV2A. In some embodiments, the sequences from gene SV2A comprise or consist of:

(SEQ ID NO: 37)
TCTCATGTTTTCTGTTTAGGGGGTGCTGCAGGATGGGGTGGGGAC
TGGCACAGAAGCTTCTCTGGGGGTAAAGGAGGTAAGCAGAATTGC
AAGTGGTTAGGGCACAGGCTTTGAGGTTATGGGTTTTGAATCTCA
GCTCCACCACCTGCCTCTGGCCTCTGGCAAATTACCTCTATGAGC
CTGGTTGTTTCCTCTGCAAAATGGAGGTGATAATAGTACCTGTGT
CATAGGGTTGATGTGCTAGATCAATGCATGTAAAGCATCAATTGC
CTTGGCCCATGGCTGGCATTGAATAAATGATGGTTACTGATATTA
TTAATAAACAATTATAAGGGAGGTTGAGGAGGAGTGAAAAGCCTT
CCTTTTGGATACCTTGGTTGGATACCTTCGGAACAAGTCTCATCC
TGGGCTATCTGAAAGCCAGCTGATGGGACAGGGTGGGCTGTGGGT
GGGTTTCACTGTGGCCAGGCTCATGTTGCTCTCTCCTCTCCTGGC
TCCA

In some embodiments, the intronic domain that promotes trans-splicing primarily in the brain comprises or consists of sequences from the gene S100A13. In some embodiments, the sequences from gene S100A13 comprise or consist of:

(SEQ ID NO: 38)
AATCGACCACGGAAATTTGACACCTCCGGGCTTGGAAGCAGCTCT
CTCCTCCTTCCCCGCTGCTTATAAACCTCAGCCCTGAGGCTCCAG
CTCACTCTACCCCATCTCCTTGCCGGGTGAGTCTCATGGCTGACC
CTGGAGGGAGGCTGGAGATCATTTTTTCTTCCTGGGCTCCCAGGT
CCCTCCCCCAGAGCCAGCTCAGACAGGAATTCAGGAAGGAGCTTT
GGATGATCGAGCAACAGCGCGTGCAGGAGTGTGTGACGGGGTGGA
CAGAAAAGAGAATGCCTCTTCCAGCCCCACCCGCCGCACCCCAGG
GGGTTGTGAGTGCCTGTACTAAACCCGTCCTGGAGCAGGGAGTCA
GAGACGCCCCGTTCCAGGACCCTTGTCACCGGGAGACAGGAGCGG
GGAGGATCTGTGGGGTCCTGGGTTCAGCACTCTGCCCTGCTGCCG
CTGAGGAGAGGTAAGGGTGGAGCGGGGCAGAGAAAATGGGTCTGT
TCAG

In some embodiments, the intronic domain that promotes trans-splicing primarily in the brain comprises or consists of sequences from the gene S100A13. In some embodiments, the sequences from gene S100A13 comprise or consist of:

(SEQ ID NO: 39)
TGGCTGTGCTGGTTGAGCCCGGTAAGGGGGGAATTCTGCTGAGGG
GGTCTTAGTCTGTGGCTCCTCCATGGGGCCTCACTGAGAGGACCC
TCAGTGAGAAACTTAGTCTTGGGAGTCTGAGAACCCACAGAGGGA
TTTCAATCCAGGACTCTTCTCTGGGGGTCTCAACCTGGATCCCCC
TCTGTGTGTGGGGCCTCAGCCTGGGGTTCTCTGAGGGATCTTGGT
CTGTAGCATCCTTTGTGGGATCTCATTCCAAGGGGCCTCTGTGTG
TGTCTCAGATTGGGCCTCTCAGTGTTGAGCTCCCTTGATTGGTTG
CCTCCCTCTTCCCCCCTCAGCATACACACTCCTGATTAGACATGG
GTATGGTGGCAGCAGCCCCCAGTATTGAGAGGACTAGGACCCTGG
GACCCCTGCCGAACGAGATCTGTCCAGGCAGCTCCTCTCCCTGAC
CAAGTGGGGGCGCTCGTGTGGCTGGCAGGGAGCCCGGGTCCAGAC
CTGA

In some embodiments, the intronic domain that promotes trans-splicing primarily in the brain comprises or consists of sequences from the gene UBAP2L. In some embodiments, the sequences from gene UBAP2L comprise or consist of:

(SEQ ID NO: 40)
ACTGAGGCACGAGAATCGCTTGAACCTGGGAGGTGGAGGTTGCAG
TGAGCCAAGATCACACCACTGCACTCCAGCCTGGGCAACAGAGCG
AGACTCTGTCTCAAAAATAAATAAATAAATAAATATAAATAAATA
AAAAATAAAGGACTTTACTTTTTGTAGTGATATGTATAAAATATT
TAAACAGAAGTAAAAGCAAACTAATGGAAGTGTTTTAGACATGGA
TGCTGAGTGGAGAATGGTTAAGTCAATCAGTCATCCCAGCTTTCA
TCTCTAGTGTCCTGATAGCACTCCATATTGATTAGATATGAATGC
TCTACCCTTCTATAGCATTCCTGGGAGATGGTCGGGAAGAAGAAG
GGAGTCTCAGGCCAGAAGGATGGTGGCCAGACGGAATCCAATGAG
GAAGGCAAAGAAAATCGAGACCGGGACAGAGACTATAGTCGGCGA
CGTGGTGGGCCACCAAGACGGGGGAGAGGTGCCAGCCGTGGACGA
GAGT

In some embodiments, the intronic domain that promotes trans-splicing primarily in the brain comprises or consists of sequences from the gene UBAP2L. In some embodiments, the sequences from gene UBAP2L comprise or consist of:

(SEQ ID NO: 41)
TCAGTAATGTCTAGACTCTAGGGATGTGCCAGGGCCCTGCTAGTC
AGGACCTAGGAACCATTATATTATCCTTTTGCTTTTCTTGCCGTC
GTCTGTTCTGAGACCTTGACATCTCTTGCATGTGGCCTTTTCTAC
CAACTGCCACCAGTCTGGCATGAGATTACCCTGTAGTCATTTATA
AAGTTAAATTTGACTTGCAGCTCTCCATTAAGGCACATTACCATT
CCCTGAGAAACATGTTCCCATCCTTCCAGCTCATTTTCTCTTCGT
TGTTTTTTTAATTCCCAATACCATATTACATACCATTTATATATT
TGTATATATCCTGTTGACTGTGCTCTGGCCATCTGTGGTTTGGTT
TTTTTGTTGTTGTTGTTGTTTTAATTTTTATTTTTATTTCAGTTC
GAGGTCAGGAAAATGGATTGGATGGCACCAAGAGTGGAGGGCCTT
CTGGAAGAGGAACAGAAAGAGGCAGAAGGGGCCGTGGCCGAGGCA
GAGG

In some embodiments, the intronic domain that promotes trans-splicing primarily in the brain comprises or consists of sequences from the gene ADAR. In some embodiments, the sequences from gene ADAR comprise or consist of:

(SEQ ID NO: 42)
GTAAGAGCCTCTGTCCTTCTCGGCTACACCTGCCTGGGCTGGAAC
GCGCGGCCCATGCGGCCTCTCCAGTCTCTGGCGTTGATGTTAGAG
GAAGCGTGGGGGCGCCCCGGCAGGGCACTGAGGGTGGCCGCAGGG
CTGGGTGGGGACGCAGCTGGTAGGGGCACAGTGGCCGGTCTCGGC
AGCCTTCCAGGAGGCGGACGCCCGGGCCGGTGTACTTTTGTGCGT
GTGTGCGCGCCCGTGTGTGCGCGAGTGTGGGCGGCAGAGGCTGCG
CACGGATGCTCCGGCGCTCGTGCCAGCCGGAGCCCAGCAGCTGGG
TACCAAAGGCCCAACAGCTGGGTACCAAAGGCCCTTGTTTCCCTC
TCGCCGGCTCCCCCGCCTCGGAGAGTGACTGGAGAGTGAGCTGCC
TGGGACTCGCCGCGGTAGGCGCTTTTGCTGCGCCTTCTACCACAA
ACTGCGTTAGGACCGGCCCTTTATCCCAGAGATAACATCGCCGCC
CTGG

In some embodiments, the intronic domain that promotes trans-splicing primarily in the brain comprises or consists of sequences from the gene ADAR. In some embodiments, the sequences from gene ADAR comprise or consist of:

(SEQ ID NO: 43)
TTCAGGATGGGAGTTACTTCTGATGGAGAGGCAGTGGCACAAGAT
GGGGACCACTTACAAGCTGATGAAAGTTAATTGATAATATTTTAG
TTCTTGGTTAGCATGTCAAGTTAAAGAAGGTGTTCATTATATTAT
TTTAAAAATGAATTCGTTTAATTTAATTTAAACGGGCCATTCAAA
GACTACTGGTTATAATGTATTATTGGCTAATTCTGTGGTCTTTTT
AAAAGAGGATTGTACCTACGTATCTTCTCAGGCCTTCCGGCCCAA
CCACGTTATAAATGACTAAGGAGTTCAATCAAGGCCATGGGCAAA
AATGCAGCATCCTAAACCCATGTACCTACTCCCCACTGTACCCAA
AACAGCGGCATTAACACAACGTAAAAAACCAAGGAAGACGTAGCA
ACAAAAACCTTAATTTCCTAGAGCGCAAACAAAAGAGGTTGAATC
ACGTGGTTCTTCCTCCCACGCCCGGCGCGCCAATGATGTCACCAA
TCTG

In some embodiments, the intronic domain that promotes trans-splicing primarily in the brain comprises or consists of sequences from the gene ARHGEF2. In some embodiments, the sequences from gene ARHGEF2 comprise or consist of:

(SEQ ID NO: 44)
GTAAGGGACGGAGCGGGAAGAGGGAGGTGTGTGTCTCGCGGGTGG
GGGCGGTGGAAGCTCCGGCACCTCCTCTCACCCCGGGCCCCACCG
GCTTGGGCTTGGCGTGGCGACCCCCCATCACTCCCCAATCGCTGG
CTTGGTCGTTCCCCCTTTGTCCCCGCCATCCGGCGGAGGGGACGG
GAGGGGGTGGTTTCCCGGCTTCCTCCCACCACCACCACCACCCCT
CCAAGACAAAAGTCAGCTCCTCCTCTCCTCGCAAATGGACTTTCC
AGCTGTGATTCAGTTCTCCCCTCTGGGTGGGCGGGTGGCTGGGGT
CGGGACGGCCGAGGGACGGCTTACTTCCCCCAAACCATCACCTCT
AGGTGAAGGGAGCAGAGGTCTTTGTGTAGCTGGGCTCCTTTCCTC
CATCCTGAGCCCTTCTGCCTGGAACATAGGGCCTCCATCCCTATC
TGCCCCGCCAGCCTCCTAGCTCTTCCTTCTCCCAACCTGAAACCC
TTAG

In some embodiments, the intronic domain that promotes trans-splicing primarily in the brain comprises or consists of sequences from the gene ARHGEF2. In some embodiments, the sequences from gene ARHGEF2 comprise or consist of:

(SEQ ID NO: 45)
ACAAACCCTGGCGTCCCGTCCTGCGCGCGGGCACCCCGCCCTCCC
TCCGCTCCGCCTACCCCCTTCCTGAGCTTGGGCGGGGGAGGAGAC
TCGGGTTTGAGGAACTGGTGCTGGGTGCCGGGCGAGGGGAGTCAG
ACTTCCTGTCCCCGAGACCAACGCGTGCGGGCCGAACCCCTCCCC
CCGCCTTCCCCCAACAATACAGGACGCCGGGGTCCGCGCCGCGTC
CTCCCTGGTCCCCCCGTCCGATTATGTCTCGGATCGAATCCCTCA
CGCGGGCGCGGATCGACCGGAGCAGAGAGCTGGCGAGCAAGGTGG
GCTCGGAAACCCCTCTCCTTTCGCCGCGGTCCTCGGGTGCTCCCC
GAACCCCGGCACCCGCGGTGCATCTCCTGTCTCCCCGCCTGCTTT
CTGGGCGCCAAAATCGGGCGGAGCGGTGGATCGGCGAGCGGCGGG
ACGGGGTGGGTGGGGACTGCGAGCGAGTGGGTAGGGGTGGGGGGA
GCGG

In some embodiments, the intronic domain that promotes trans-splicing primarily in the brain comprises or consists of sequences from the gene PAQR6. In some embodiments, the sequences from gene PAQR6 comprise or consist of:

(SEQ ID NO: 46)
AGATTCCTCTTCTGGAGTTTATTTGGGAGCAGCTGGGATGATGGG
GACCCCACATCCATAGGGCTGGGAGGTCAGGGCAAGGGCAAGGGG
AAGAGGAAAGAAGGGTGCCTGGAGAGGAGCAGAACTGGGGTTGCC
GGCCAGGCCAGCAGAGCGACACCCTAGACCGGGCCGTAGAAGCGC
CGATAGGCCTCCTGAAAGCCGATGTGGTCAGCCAACTCGTCACAG
TCCGGATTGAGCTCACACACCTCCCTCCTGGGCTCCAGGGGATCC
GGGTAGGGGACTGGGGCTCTGAGACACCCGACGTGGTGGCATCAG
CCCCCGTGCAAAATGCCCATCCCTCCTCTCCCTACACATGGGGCT
CCAGGGTGCCCACCAGGGCAGGCTGCCTGTACCCCCACCACAACC
CAGAGAGGCCACTCCCTGCTGCAGCTCCCCCACAGACCATCCCGG
AGAGGCAGGGCCTTGGCCCAGCTCTGCCTTTTCTCTCACCCCAGC
CATT

In some embodiments, the intronic domain that promotes trans-splicing primarily in the brain comprises or consists of sequences from the gene PAQR6. In some embodiments, the sequences from gene PAQR6 comprise or consist of:

(SEQ ID NO: 47)
GTGCTGGGGCAGGGGCCACGGCTGTGGGCAGGAGGCCCTGAGCAC
CAGCCATGGCTACCATCTCTTCTGCGCGCTGCTCACTGGCTTCCT
CTTCGCCTCCCACCTGCCTGAAAGGCTGGCACCAGGACGCTTTGA
TTACATCGGTGAGGGCACGCCTGGCCCGGCCCGGGAAGAGGCAGG
GGCAGATGCCTTCCCAGAGCACAGAATGAACTGGGTAAATGGGTA
TTACAGCCCCAGGATGGAGGCAAATTATAGGAGGGACTTCCCTGT
CTTCCTATAACGAGAGCATCACTGGAAGGGTCCTCCACCTGCCAC
TTCTCAGGTAGCAAAACTAGTAGCATGTGTGCTGGGGCCACATGC
ACACATGCTCATTCATTCCTTTCAGTCACTCTGGGAGTCAGGGTT
TCTTATTCTCAATTTATGGGGGAGTAGCAGGCCCAGAGGATGGGG
TGACTTGCACTGGGAAAGGTGTGGCTCTGACCTGCCCATCCTCTC
CACA

In some embodiments, the intronic domain that promotes trans-splicing primarily in the brain comprises or consists of sequences from the gene CADM3. In some embodiments, the sequences from gene CADM3 comprise or consist of:

(SEQ ID NO: 48)
AGTATCTCATGAGTTATCTTTCTCCGTGAAAACCATGGGCTAGAG
AAGGGGACTGCAGACACCGAGGGGAACTGTTCCTGTCATCCAGAG
GCTGAGAAACATGGTGGTCTGCTGTTTGAGGATGTAACAAGCCAT
GAGTTCCCCAACTGGTTCCAGTATTGCAGCTCTGATTTACAGCAT
GCATAATCTTACAGGGTTCTGGAGTGACTTCACCCCACAGGAGGA
ACTCTAACAGGAAGCTGAAGTCAGTGTTGCCCTAGGTATCTGACA
ATGAAGCTCACGAAATCCAAACTATTTTCTGAGTAGCTCTTAGGC
TACTATGTCTAATGACCCAGAAAAGTGATTGAATAACTTAAAGAC
TGCTTAAACTCAGGAGCAGTTATTTTTTCCCCACCCACCATACTA
GACCCCTTCCCCCAAAGAAGCTGGCCTGGGATTGGAAGGTAAACA
TGAAAGGTGCCAGTAAAATCCTAGCTTTCCATTCTCTTGATTTCC
CCAG

In some embodiments, the intronic domain that promotes trans-splicing primarily in the brain comprises or consists of sequences from the gene CADM3. In some embodiments, the sequences from gene CADM3 comprise or consist of:

(SEQ ID NO: 49)
TGAGACTCCCAAACCCCAGTGTCTCTACAGCATGCTTCCTTGCAA
ACAAACCTCCCTAGATGGGTCCCTGAAGCAGCTGGGAGCCAGGCA
AGTCTCCAAGCACTTTAGGAAAGTTCAGCCTGTGTTCCCCTGGCA
ATGGGATAAAATGTAAAAAGAAATGGGTTTTAATCCTTTGTGCTC
TACTTGCTGTTTGATCTAGGACAAGGTTCTTACTCTCTTTGAGTC
TGAATTTTCTCTTATAACAGAGAGATGATCATACCCCTTCTACTT
ACCTCCCAAGATGGTGTGAGGAAAACACAAGACATGCATGTGTTA
TAATTTCCAAAATAAGTACCCCAGTTGCTTACATGCTCTCATCTT
CATCCTATATTTCATCCTATATTTCAACTTCCTTCTTTGCGTCTC
TGAGACTCACACTATTTCTGTGGCCACCCAATTGTGCTGTAAGTG
GAGGGTCATATATCATGCCTGTGCTATGGCATTGCTCCTCACTGC
CATG

In some embodiments, the intronic domain that promotes trans-splicing primarily in the brain comprises or consists of sequences from the gene NDUFS2. In some embodiments, the sequences from gene NDUFS2 comprise or consist of:

(SEQ ID NO: 50)
GGTGCCCAGGGGTCTGGCTTCTCCAAACTCTCCTCCTGAGCCCTC
CTTTCCTGGATCTTTGTCTCTCCCTGCCTGTGTCTTCTGCAGCTT
CTCTGGCCTCTCCCTCTGTAGGACTCTGTCTGGGCCGCTTCTGTG
CCTGCCCTGTCTCTGCCTTCTTCCGCTGTGCCTTTGTCTCTGTCT
CTTTCCTCCTCTGTCTCTCTCGTGCATATGTGTCTGTGGGTCTCC
CTGTGGGTTCTCCCCAGCCTCTCTCCGCGCCAGCTCTTTTAAGCA
TGGTGAGCCTCTGCGATTGATTGGCTGATCTCTAAGCCACTCAGC
TGAGCTCTGTCGGTGGGACTCAGATGGGTGTATCATCGCTTCCCA
AAAAAATGGGACTTGCCCAGGGGAGGGGAATCTCTAGCAGCCGAA
TGGATAATAGGCACTGGGCCAGCTGATGACATAGCCTTTTCCCAA
ACCCCACAATCAGCCCTCAGCTGTCTCTTTCCCTTTTTGGTGGGA
CAGA

In some embodiments, the intronic domain that promotes trans-splicing primarily in the brain comprises or consists of sequences from the gene NDUFS2. In some embodiments, the sequences from gene NDUFS2 comprise or consist of:

(SEQ ID NO: 51)
TAAGGAGGCATCAAGAGGGAAGAACAAATAAAAGAGGTAGCTTGA
CATGGCTCAGGATAGAACTGAGCAGCTGGGAAGAGTAAAAAAGGC
AGAACATTGGGGGAGGCAAGGAGACAGTGATCAGGGAGTTGACTG
CCCAAAGGTAGGATGATCCTGCTTGGAATGAACATAACAGAGGCG
GCAGATGACAAGGCAGGAGGGGTTAACGACTTTTCCTTTTATAGA
AGGCGGATCCAGGGTGGCACCTCCTCTTTTCTAACGTCCCTTCTC
TCCCTCAGGATCCCCCTTCCTGAGTTACCACTACCCACCCCCTCC
AAAGCCCAGAACAGCCATTCCGCCTCCATTTCCCCCCAGGGTTCC
CCCTCCCCACTTCCTGACTCCCCATTTCCCTGGCCCAGCTCCCAG
TAGCTTCCCCCAGGCTTTTGGCCCTGCTCCTACCCACTCCCAACT
GCCCTAAACCCTCCCCCAGCCTCAAGTCAGCCAGCCCCCAGCATA
GGCA

In some embodiments, the intronic domain that promotes trans-splicing primarily in the brain comprises or consists of sequences from the gene DHX9. In some embodiments, the sequences from gene DHX9 comprise or consist of:

(SEQ ID NO: 52)
GATCAAAATGATATTTAGAGCATGGTTCCCCCTTCCTCCCACTGT
TTTGTTCAATCCTGAATTCTCAGATATTTCTGGTGTCAGCCTTTT
TAAAACCGTCAGGTTGCAAAGGAGTGATACATTTGTTGATGATTT
GTTTTCTTTCATATTTTGTCTTTCTGAACGCATTGGGCTTGGTAG
CACTTGGTGACTTTAAGAACAATGAGATACCGAAGTGTGCCTGAA
GCTCATCGTTGCAGACATTTTTTGTTATCAGTATCTTATCATAGA
TTTCTGATACAAAGCAGTGAAGAATTCAGGAAAAGACAATGTGGG
AGGCACAGACAGTTGGGGGAGCCAGGACAGCATTGCTCTTTGTAT
GTCCTTTTATTTAAAGGCATAACAGACAGCAACACCAGAGGTCTG
TGTTCATACACACATGTGCTGCTGCTTAGGCTGAACTAAGCAATC
ACATGGGAAACTATTGTAAACTGATTTCTGGTGTCGCTGTTCCCC
CTAG

In some embodiments, the intronic domain that promotes trans-splicing primarily in the brain comprises or consists of sequences from the gene DHX9. In some embodiments, the sequences from gene DHX9 comprise or consist of:

(SEQ ID NO: 53)
TCAGTTCACACGAGGTATAGGAAGGAGTCTAACATTTATTTTTTT
CTTAAGCCTCACCTAGCCTTTTATTCACACCACCAAGTCTAGGTT
AGAATGTATCTGAGCAGTGAAAGGACCAGAATGCATAGTTATCAA
AGGGCAGTTTTTCCCTTGCTAAAACCTAAATTCATTTTTTAGACT
CTGCTAGCAGTTTTAGATATAATGTTACAACATGTGTTTTGTTCT
CCAGGACCTGATTTAAATAAATGTGATAAAAATAGGCTAAGTTTT
TTGTCATCCAGGGCTGCTTCCAACTGCAGGCAGATGTGAGAACTC
TGCCTCAGGAGCCACTGATATGCACAAAGTGTATGTGCATGTGTG
AGTTTGCGTATATTAGCTTAATAGATCGCAGTCATTAATGGGTGG
CCTATGCAAAGGAATCTTAAAATAATTATAACCGTAATGTAAAAC
ATTTGGGGCCTACTTTATTTCTTGGCTCCAAGCTAAGTGGAAAAT
TCCT

In some embodiments, the intronic domain that promotes trans-splicing primarily in the brain comprises or consists of sequences from the gene TMEM9. In some embodiments, the sequences from gene TMEM9 comprise or consist of:

(SEQ ID NO: 54)
GTGAGGTCTGATTATTTGCCCTGTTTAAACAGGCGTGAACGCGGT
GCCCCAATCAGGTGGGACATTGGTCCAGTGGAACTTGGCTATTGG
CTGAGCGGGGCACCCAGCTTGAAAGTCCTGAGGTTCTTTTTTTCA
CTCTTCTTCACAGTCTAGGTAGGGCAGGGCTGAGATCAGAAGCCG
GCAATGGCCAGGCATTGTGGAATTAGGAAAAGCATTTGAGAGGGA
GTCAGCAAGTCTGGGTTTGAGTCACACCTCCACCATCTGGTAGCC
TTGGGATCACAGGTGATCTAGCCCAGCAGTTCTCAATCAGATGAT
TACAGCCCCCCCAGGGGGCATTTAGAGGTGGGTGAAGGTGCTTTT
GTGGTCCCAATAACAGGTGTATTACCAACACTGTTACAGGGACCA
TGGATGCTAAATGTCCCACCCAAAATCCCAGAGCACCCACATTGA
GAAACATTGAATTTGCCTCATTAAATCTGTCTTTTCTGTAAAAAC
AAAA

In some embodiments, the intronic domain that promotes trans-splicing primarily in the brain comprises or consists of sequences from the gene TMEM9. In some embodiments, the sequences from gene TMEM9 comprise or consist of:

(SEQ ID NO: 55)
TGCACATCACCACACCCAGCTAATTTTTTAAATTTTTGGTAGAGA
CAGGGTCTCACTGTCTTGCCCAGGCTTGTCTCGAACTCCTGGCCT
CAAGCACTGCTCCAGCCTTGGCTTCCCGAAGTGCTGGGATTATAG
GTGTGAGCTCCATGCCTGGCCTAATGTAGCCCTTTCTGTTCCAGT
TTGTAGCATTTTTCTGCTTGTCCTTTGAAGAGTCTATAAATAGCA
TTGGAAAATAATTCTCATATTTAAAGGCACTGCTGGGTCCTCCAT
CAGGTTTCTCCTTTCGAGGCACATTTAGGGGGCGGTGGTCTTCAC
TGCTGTCCTTCCTGCCTATTCCAGGGAGCCCTGTGCTGTAGCTCC
TCTGTGTTCTCACCACTCAGGGAAAAAGCAGCTGTCTGTCCTTCA
CTCCCAGCCAGTGCCCTGTGCAGACCCAACTCCTTTCATCCACCT
GAGGCAGCCCCTCCTGCAACTGTCATTCTCCTTCTCTGAACTTGC
TCCA

In some embodiments, the intronic domain that promotes trans-splicing primarily in the brain comprises or consists of sequences from the gene GUK1. In some embodiments, the sequences from gene GUK1 comprise or consist of:

(SEQ ID NO: 56)
TAAGTACTTCCCTAAGGGGCGGGGAAGAACGAGTGAGACATGGGC
GGGGCAGTCGCCTCATGACGTCAGTCTCGCGCGCGGGCGGAGGTG
GGCCGGTGCGGCGCTGTCACGTAGGTTCAGTGGGCGGAAGAGGTG
GCCCCGGATGCTGCGGCGCCCGCTGGCCGGGCTGGCTGCGGCCGC
CCTGGGCCGGGCCCCACCGGACGGTGAGTACGACAAGCGCGATCG
CGAGGGTGACTCGGGTCGAGGCGGCAGCGGTCCCTGCGCTTTGCG
GTCGCCCAGTCCACGCCGCCTCCGGATCTCCTCCTAGCCGAGACG
CCCTGTGCCGCATTCAACTGTGGGCCCTGAGAACGGGGGAGCTCT
GGAGGGCGGACGGGCTGGCAACGCAGCGGAGCCCGGGTCCCGGAG
GGCGGGGCGCTCCTCAGGAGCTGGGCAGGCCGGCAGTTATCGCAG
TGTCGAGCTGCTCCCCGGGCGTATAGGAGCGCTCGGTGTCTTCCC
GACA

In some embodiments, the intronic domain that promotes trans-splicing primarily in the brain comprises or consists of sequences from the gene PDIA6. In some embodiments, the sequences from gene PDIA6 comprise or consist of:

(SEQ ID NO: 57)
GTAAGTAGAGGAAACTCCCAGAGTAGGAGCCTCCATCTGTTCACA
ACAGCCAAAAGGTGAAAACAACCCAAGTGTCCACTAACAGACGGA
TGGATAAACAAAATGAGGTCTAGTCATATGATGGAATGTGACTCT
GCTTTAAGAAGGAAGGAAATTGACACATGCCACAGCATGAATGGA
CCTTGAAGACGTTAGGCTAAGTAAAATCAATCACTCACAAAAGGA
CAGTACTATAGGATTCTACTTACATGAGATGTCTAGAGTAGTTAA
ATTTATAAAGACAGAAAGTAGGATGACAGTTGCCAGGGGTCAGGG
GGAGGGGGAATGAGGATTTGGCATTTAATGGGGATAGAGTTTCAG
TTTGGGGTGATAAAAAGCTCTGGAGATGAAGGTTGTACAACGATG
TGAGCGCATTTAATGCCATGGAACTGTACACTTAAAAATGGTTAA
AGGCCAGGTATGGTGGCTCACGCCTATAATCCCAGCACTTTGGGA
GGCT

In some embodiments, the intronic domain that promotes trans-splicing primarily in the brain comprises or consists of sequences from the gene PDIA6. In some embodiments, the sequences from gene PDIA6 comprise or consist of:

(SEQ ID NO: 58)
CTAACCAAGCAAGCCAACAGGATTACCATAGGGTGTTGGAGAAGG
GAAAGTTGTAGGTAAGATTTAAATAAAGCAGTGTTTAATAGGCTC
AAAGCAAAACACAACTGTGTATAAAGGAATTACGGTCAGACCTGG
GCTACGAAGTGGATGGAGGTTCCTCTTTCCTTGGAAATGATGAAG
TTAAGACAAATGTGCAATGTTGTGTCCAGAAGGCCCTGGGTTGGA
AATCAAGGCAGCTTCTCAGTGTTAAGATGACCCAGGTCCTTCAGA
CAAAAGAAGGATGTTCCATTCTTACTCTATTTATCTCAAATAGCA
CAGTTTATAACAGTCTGTGCTTTTTCAGGAATAAGATTTTTCAGC
ACTATAACTTTTCGTTGATTAACAAAAGCAGTTTTAGCAGGTTCA
CAGATGTAAATACGTGGAGCCTGGCATATGGTGAACATTCGGTAA
TAGTTTCCCCATCCCCACCTCACTCATCTCCTCTTCTCTCTCACT
TTCA

In some embodiments, the intronic domain that promotes trans-splicing primarily in the brain comprises or consists of sequences from the gene SNX17. In some embodiments, the sequences from gene SNX17 comprise or consist of:

(SEQ ID NO: 59)
TCTCAAACTCCTGACCTCAGGTGATCCACCCACCTTGCCTCCCAA
AATGCTGAGATTACAGGCATGAGGCCTGTAGAAACTGATTTCTTT
GGGCCTCAGTTTCTACAACTGAAAGATGGATTCTTTGATCTGCAA
AGTCCCTTTAAGTCCTAAGATATTTTGTGAACAGACTTAGCTATT
AACTGTGCACTTTATAAAGCCCTTTCCTTCCTTTAAGGGTTCAGT
GAACAGTCTTGCTAGGTGGTCAAGGGGCTCCCTATAGGTCCTGGC
CCAGTACAAATCCTCCCCGTACTGTGGAGGGGCTGACTTGTACAC
AGTAAAATAGAGCACAACTTATATCTAGGACAGCTCCCTTGAGGT
GCTACCGCTGTCAAAGGAATTGTTTCTCAGATGACCAGGGTAGAC
AGGGGAACAGGGACTGAGGGAAGGGAGGGGCACCCAAGAGAACAT
TATGAGCATATGTAGATTGCCTCATCTCATTTTCTGTGTTTATGT
GAAG

In some embodiments, the intronic domain that promotes trans-splicing primarily in the brain comprises or consists of sequences from the gene SNX17. In some embodiments, the sequences from gene SNX17 comprise or consist of:

(SEQ ID NO: 60)
TTCGGAAGGAGTATGGGGCCAATGTGCTTCCTGCATTCCCCCCAA
AGAAGCTTTTCTCTCTGACTCCTGCTGAGGTAGAACAGAGGAGAG
AGCAGTTAGAGAAGTACATGCAAGCTGGTGAGTGGTTGCAGGAAA
CTAGGTTGACTATATTGAGGACTATGGGGAGAGACTTAAAAACAG
CTGGTTCTGTGGAAGGGCCTGATTTAATTTCTAGATCTAGGAAGG
CTCTTGTTTGATCTGTTATGCAGTTAGCAAAGTAAATAGTGTACG
AGGATGTGTAACTCTGTGTACTAGCAAAACAATTTGGGCACTAAC
ATCTATGAGAAATACTAGTATAACACTAGTCTTAATATCAGTGGA
GAGAACTTAATTTGTATCTGATGTGCTTTAGATTAACATCATTGT
TATTTCCCTAGTTCTATAGACTGGACTCACCTAGATGGCAGCCAA
GGGTGGGGCAGGTGAAGAATTTATGCAAACAGTGAGTGAGGTGAG
GCCA

In some embodiments, the intronic domain that promotes trans-splicing primarily in the brain comprises or consists of sequences from the gene DCTN1. In some embodiments, the sequences from gene DCTN1 comprise or consist of:

(SEQ ID NO: 61)
GTAGAGACCCACTCTGGCCGGGGGTTACCAGGCTGGTGGGAGTTG
GAGCTAAGGGAGCAGGGTGGAGGGGGAAGCCAGGACTGGAGATGG
GTGATGGGGGGATGAGGAAAGGGGAAGAGTAGGAATGGAGCCCAA
TTTTCAACCCTTTCCACTTGGGGCTGATGAGATTTTTTATGGTCC
CCACAGGAGCCATCCCTGGGCAGGCTCCAGGGTCTGTGCCAGGCC
CAGGGCTGGTGAAGGACTCACCACTGCTGCTTCAGCAGATCTCTG
CCATGAGGCTGCACATCTCCCAGCTCCAGCATGAGAACAGCATCC
TCAAGGTGAGGGAGCCACGGGGAGGACTGGGATGGGAGATGGAGC
ATATTTGCCCCATTGATGATAGATGACAGGGGCCTCCTAGCACCT
CTTACCACTATCCTTCTTTTTTCATCCACAGGGAGCCCAGATGAA
GGCATCCTTGGCATCCCTGCCCCCTCTGCATGTTGCAAAGCTATC
CCAT

In some embodiments, the intronic domain that promotes trans-splicing primarily in the brain comprises or consists of sequences from the gene DCTN1. In some embodiments, the sequences from gene DCTN1 comprise or consist of:

(SEQ ID NO: 62)
AACATAGTTCTGAATTTATGAAACTTCCCACCAAAAAAGAAGGTC
TTTTGCCTCAGAGCAATGGGGAATCCTTAGGAGCAAGGGAGTGTG
TTTGCATCCCCAACTTAAGCTTTGCTGCTGGTGAGCACTAAGGGT
GCAGACATATTGGGGTCTCTGGAGCATTCTCCGTGCCCTTCTTGG
AGATGGCATCTGAGACAGGGAGGCAGGAACAGCCTCATGTTCCAA
GTACTTTGTACAGTGCACACTCTCTTTGCCACTGTTCTTCACACA
TTGTACCCTGTCTTGTGGCCTCACTCCTGTTTGGTGTCTTTCTTT
AACACTTATCTGCTCGTCCCCAAAGATAGGTGGCAGAGAGTCTTG
TTAGAGAAAGTATGATGATTAGGAAAAGGGTTTTCCTGCAGGTTG
TGTCCAGGATTCCTCTGGTGTGTCCCCGTAACCCCCAAATCACCT
AACTCCTCTCTCCTCTTTTCCTGCTCCCCCATGGGCTATCCCGAG
CACA

In some embodiments, the intronic domain that promotes trans-splicing primarily in the brain comprises or consists of sequences from the gene WDR54. In some embodiments, the sequences from gene WDR54 comprise or consist of:

(SEQ ID NO: 63)
GCTCGCCCCGCCCACAGTTGCCCCGGCAACCGCGCCGCCCGCGGC
ACGTTCCGGCCGCTCACCCCGCCCAAGGGCCGTGCGTACGTGCGT
CGTCTCTATGGTGGCGGCGGATTTGGAGGGACCCTACGAACCAGG
AGTCAGGCGAGCCGATCTGGGGCTGCAGGTGTTACCTCTGATCTA
GGCCGGGGGCTTCAGGGATCCGAGCCGAGGGAGAAAGCCTTGGGG
GCTTCATCACACTTATTTGGCTCGCGGGCTTGGGGCCATCGGGTG
GTCCTGTGTCATCCCCATTCCTCCTCTGCGATTCCCCCGTCGCCC
CAAAACAGCCCTGGGACTCCCCTGTCTTGCTCCTCGAGACCCTCC
CCCATCACCGTCCTCTGGGCACCCCATCCTATGACCCAGTCTCAG
GCATCTTCCGAAATGCTCGATGATAGCCGCCCTCGGGCATCTCCG
CTGCGTCTGTTTGTAGCGCGCCTGGTGATTCGGCTGCACCCCCAC
ACAG

In some embodiments, the intronic domain that promotes trans-splicing primarily in the brain comprises or consists of sequences from the gene WDR54. In some embodiments, the sequences from gene WDR54 comprise or consist of:

(SEQ ID NO: 64)
TGAGGCATGGAGCTGGGAGTGATGTTGCTGAACCCAGTCCTACCC
CCAGCCTTTTCTCTCCAAACCTTCAGGAGCAGGCATGTCCTAACC
TCAGAATCTCCCCAGGCATGTCCTAACCTCAGAATCTCCCCTCTC
CTGCCGGGCGCGGTGGCTCACGCCTGTAATCCCAACACTTTGGGA
GGCTGAGACGGGCACTTGAGGCCAGGAGTTCGAGACCAGCCTGGC
CAACATGGTGAAACCTCTTCTCTACTAAGAATACAAAAAAAATTA
ACCGGGTGTGGTGGCGTGCACCTGTAATCCCAGCTACTTGGGTGG
CTGAGGCAGGAGAATCGCTTGAACCCAGGAGGCTGAGGTTGCAGT
GAGCCGAGATGGCGCCACTGCACTCCAGCCTGGGCCACAGAGCAA
GACTCCGTCCCCCAAAAAAAAAAAAAAAACAAACAAACTCATCTT
CCCTGCTTTGATTTTCTCCCTACACTGATGCACCTCCCTCCAGGT
CCAC

In some embodiments, the intronic domain that promotes trans-splicing primarily in the brain comprises or consists of sequences from the gene CAPG. In some embodiments, the sequences from gene CAPG comprise or consist of:

(SEQ ID NO: 65)
GTAGGAGGAAGAAAGGAAAGTAGGAGTGTTGAAAGGTGAAGGTGG
CAGGGTGGCTGCCAGCGCGGCTGGCTGGTACCCTCCTGGGTGGGC
AGGCGAGGGCTGGCTGTGGTGGCCTGGGAACAGAGACCCACATTT
CTGGATACCACATTGTGCCAGGGTGGAGTTCCCCCAGCCTGCTGT
CCTGCCAGCCTTATGCCAGGCTCCAGGCACCTGATCTGATGGCAG
CTGTGAAGCTGCCCGGGAGGATGGGTGGGCTGTGCCCATGCTGCA
CATGGAGGAACTCTTGGGTCAGGGCTTGTCCCAGCTGTGCCAGAC
CTCCTGACGGAGGCCACCACACTGCTCCTGAAGGCCCTCAGCCAG
ACTGGAGGTGAAATCAGATTAGAAGCTGCCTGTCCCCAAGACAGG
ACTGTGGTGTGATGCTATTTGGGTGGGAGTGGCTTGGGGAGGGGG
CAGGGCAGTCCTAGCCATTTGAATTCAAATGCAAGTGGCACATTT
GATT

In some embodiments, the intronic domain that promotes trans-splicing primarily in the brain comprises or consists of sequences from the gene CAPG. In some embodiments, the sequences from gene CAPG comprise or consist of:

(SEQ ID NO: 66)
AGAATGTTTTATGGCCTCAGCCATTCCCTGAGAACTGAGCTTAGC
CAGGTGCAGTAGCCCACGCCTGTAATCCCAGCACTTTGGGAGGCC
AAAGTGGGAGGATTGCTTCAGTTCAGGAGTTCTAGACCAGCCTGA
GTAGCAAGACTCTGTCTCCAGAAAAAATAAAAAAATTAGTCGGGC
ATGTTGGTGCAGGCCTGTGGTGTCAGCTGCTCAGGAGGCTGAGGC
AGGAGGATCGCTTGAGCCCAGGAGTTCAAGGCTGCGGTTAGCGGT
GATAATGCCACGGCACTCCAGCTTGCGTGACAGTGAACCCTGTCT
CAGGAAAAAAAAAAAAACAAAAAAAACCAAAAAACTGATCTTAAA
AAAAATCAAATACTTGCATTCAGGGCAAGGAGAAAGACAAACTGA
GGGAGGAGGAAAAAGTAATGTGAAGCCAGTGACCCTGCCCAGAAA
ATGTGACTAACCCAGCAGCGGTGGGGGCTGTGCCGCGCCTGCCCC
AGGC

In some embodiments, the intronic domain that promotes trans-splicing primarily in the brain comprises or consists of sequences from the gene DBI. In some embodiments, the sequences from gene DBI comprise or consist of:

(SEQ ID NO: 67)
CAGCAAAACTAAGCACCCTACTTCCGTTGTCCCCACCTGTTCCCG
GCGTCCCCTTCGGCTACTCCCGGCGTTTGCGCAAGCGGTCCCACG
TGGGCTCGGGCGGGGCTAGCGCCGCGGCGGGGGCTGGGCACGCCC
CTAGCGCATAGCTGGCTTCTGATTGGCTTTCCGGTGCTCGCCCGA
GCAGGGTTGGGGCGAGTGGACCGCGCCTCTAAAGGCGCTTGCCAG
TGCAATCTGGGCGATCGCTTCCTGGTCCTCGCCTCCTCCGCTGTC
TCCCTGGAGTTCTTGCAAGTCGGCCAGGATGTCTCAGGTACAGCG
CGTGCACAGCCAGGCTGCGAAGGTGCAGCGGGCGGGAGGCCCGTT
GGGGGCTCAGCCGGCTGCCAGAAGCTCTCGGGCTCTTTCCTTCCG
TGCCCCTCACTTGCTCATGGGCCCATGCCTAGCCCTGATTCGTTG
GACAGAGCCTTGTGAGCGGGATTTTCCGTTTGGGGATTTCTAAAT
CTGC

In some embodiments, the intronic domain that promotes trans-splicing primarily in the brain comprises or consists of sequences from the gene DBI. In some embodiments, the sequences from gene DBI comprise or consist of:

(SEQ ID NO: 68)
ACAGGGCAGGACGTCGCGGCGGAGTGGGGAAGCGAGGAGTCCGTG
GCCGAGAGCTTGGAGGTCAGGGGAAGTACGGGGCCGGCTGCTCAG
AGTGCGGGACGAGGAGAATCGCGGCCCGGGGAGAGGTGACCCAGG
GGCCCCTCCCTTCTCTCCAGTGTAGACCCTTGTCTGAGACCGAGC
TATGTGGGGCGACCTCTGGCTCCTCCCGCCTGCCTCTGCCAATCC
GGGCACTGGGACAGAGGTCGGTGTTGAACGCGCGGGCCCCAGGGG
GAGGGAGGGGACCAACGGGCTCCGGCGCTGACACCGCGGCACTCA
TGCCCTGTCCCCTTTCAGCTGTTTCCAGCATACTGTGCCCCGTCT
GTCCTCAGGCCAGGGCTTCGCTGCAGCCCCGGCCACTCCCTAGTG
CCTGGCCCGGTGGTGGCCAGGCAGTTGGCCGCGCTGCTTCTCCCG
CAGAGGGGACCCCCACTGGGGGCGAAGGCTTGGCCTGCCCTCTTC
ACTG

In some embodiments, the intronic domain that promotes trans-splicing primarily in the brain comprises or consists of sequences from the gene BIN1. In some embodiments, the sequences from gene BIN1 comprise or consist of:

(SEQ ID NO: 69)
GTAAGTGCCCACCTCCAGCCCCTGTCTGGCTTGTCCCCAGTCTCT
AGGGGTGCAGCATGGAAGGAGAGCCCCGAGGAGGGGTTGCAGGAG
GGACCAGGCCACCATGGATGTGAGGGTGAGGACAGGGTCCTGAGC
TAGGCTGCCCCAGCACGGGCTTGTCACCAAGGCTGCCAGGGATGA
GTGAGCGCAGCTGGGCGCATCAGCCCCTCCTGCTTGCCCACCCCA
GCCCAGCCTCCCAGCGCAGGAGGAGCATTTAGAGCACCTACTGGT
TTATGCCAGGCACTTTCCAGAATCTTCTCATTTAATCTTCGTCAT
CACCTTTACAGCCAGACACTTCCTCCTTTTACAGATGAGGAGATG
GGGGCTAGGAGGGTTGAGTAACTTGGTTCCAGCACTCAGAAGTGG
CCAAGTCTGAGTGTCGGCCCAGGTCAGCCCAGCTCTGGGGTTCCT
GCAGGGCCTGCCGTGGTGCCCGTGCTGCGGTGCCCGTGCTGCGGT
GCCC

In some embodiments, the intronic domain that promotes trans-splicing primarily in the brain comprises or consists of sequences from the gene BIN1. In some embodiments, the sequences from gene BIN1 comprise or consist of:

(SEQ ID NO: 70)
AGAGGCTCAGGGAAGGCTCAGTACTTGCCCAAGAACTGGTCATGG
TAGAGCCAGAATTCCAACAGGGTCTCCTGGGCTCTGTCCCTGAGA
CCCCCTGATACAGGCAGAGATGCTGGGAGGGGCAGGCGGGTGTGC
AGGCGCCCTTGGGGCATGCGCTGGCAGCCCAGGCTCCTGGGAGCT
CTGGAGGCTCCACCGCAGGATTTCCCTCTGGAGGAAGCCAGAAAG
AGCCAGCCTGGTGCGAGCTGGTAGGGCCATTTTGACAAGTGGATT
TCGGTAGGTGCTGAGCTTGGGCAGCACAGTCACACCTGCCTGTCC
CTTTGACAGTGGTAGGAGAGAGGATGTGGGAGGCGGGTGGCTGGC
CGGGCTCCGCTGGTACCCACCCTGCCCCCACCAACCCCAGCCGCT
GGTGACATTTTCTCTTGTCTTGTGATCCTGCCCATTGCCTTTCCA
CCCCGGCCTCCCCGCCCCCTCCCTGTTCTCTCCTCGTGGCCTGTT
ACCA

In some embodiments, the intronic domain that promotes trans-splicing primarily in the brain comprises or consists of sequences from the gene KIF5C. In some embodiments, the sequences from gene KIF5C comprise or consist of:

(SEQ ID NO: 71)
ACTGCTTTCTTTTCTCTCTGGTAGGTCAGCAAAACTGGTGCCGAG
GGAGCTGTTCTTGACGAAGCTAAAAATATCAATAAGTCTTTGTCT
GCTCTTGGAAATGTGATCTCTGCTTTGGCAGAAGGGACAGTAAGT
GATCCTGCCCCCATCTATTAAGTAATATTATGAGAAACCACCTTT
TGGGGCCTCATAGTCCCTTTGCCACACACCCCAAAGGCTGGTTTG
TTTTTCTGTTTGGAAGAGGACAGAGGGAATCCTGGCTATGGATGC
CTGCTTGCTGGGAGTTCCCATCTACTAAAAAGTATGAATGGTGAT
GCTAATTTCCTATGACATAGGTCAAAGAATGGGCATTCTAAACTT
TGGCAAGATCCAAAGACCCAGCCACAAATTCTTGTTTTACTCGGT
TTCTAAGCTCAGTGTCTTCATCCCTGACTTGCTTGCCTCTGTGTT
TTTCTCAGAATGGGCTGTCAAAACCAATACTTTTTCTTTTCCTAA
ATAG

In some embodiments, the intronic domain that promotes trans-splicing primarily in the brain comprises or consists of sequences from the gene KIF5C. In some embodiments, the sequences from gene KIF5C comprise or consist of:

(SEQ ID NO: 72)
TACGTGTGGTCTCTCAGGACCCATCCTCTGTGCTAGGTCTTGGGT
CTTAAGAGTGAATGGCAAGGCTGGGTGCAATGGCTCACACCTGTA
ATCCCAGCACTTTGGGAGGCCAAGGCGGGTGGATTGCCTGAGGTC
AGGAGTTTGAGACCAGCCTGGCCCACATGGTGAAACCCTGTCTCT
ACTAAAAATACAAAAATTAGCCGGCCGTGGTGGCATGCGCCTGTG
GTCCCAGCTACTTGGGAGGCTGAGTCAGGAGAATTGCTTGAACTC
AGGAGGTGGAGGTTGCAGTGAGCCAAGATCATACCACTGCACTCC
AGCCTGGGCGACAGAGCGAGACTGTCTCAGGGACAAACAGCCTTT
TACTAAATTACATCTAGCAGGTGATATGTAAACTTGAGGCATCAC
TTATTGAAGCCAAAGCAATAATTATATTTTAAAAT

In some embodiments, the intronic domain that promotes trans-splicing primarily in the brain comprises or consists of sequences from the gene DYNC1I2. In some embodiments, the sequences from gene DYNC1I2 comprise or consist of:

(SEQ ID NO: 73)
AGCCCACTGTTTTTACTGCAGCGTGTATAAACATTTAGACATAAT
TTTAAGGTTCAATGAATGTGACTATCCTAAAAAACACAGACTTTT
AATTAAATACTTATTTCTTTAGTTCCTATATAACTAGGAACTTGC
CTTCTTTTCAGTAGACTGTTAAAAGAAGGGCTTTCAGCTCAACAT
AAACTGTATTAGTCACTTAATATCATACACTGAAAAATTATTTTG
AAAAGATACTTGGGATTGGGGACATTTATGATAAAATTTAACCAG
AAGATGGCAGTGATGATCCTCCTATACTCCCTGCAACGGTAATCA
AAAAGTTTTACAAATGTCATAGCATGAAATGTGCACGTTCAGCTG
TTGTTATATAATGTTAAAAGTTGAGGATCTAGGGGAAAGCACTTG
CTGTTTATTTTATATACTATGAAAAATGTGTATTTTTCTATAAGA
AGGGATTAAGAATATTTTGGGTGTCTGTATCTTTGTCTGTACACT
TCAG

In some embodiments, the intronic domain that promotes trans-splicing primarily in the brain comprises or consists of sequences from the gene DYNC1I2. In some embodiments, the sequences from gene DYNC1I2 comprise or consist of:

(SEQ ID NO: 74)
TAAGGAAGTTCCCATAAGTCTTGCCTTGTTTATTTGCATGCATCA
CTTTATGTTATGCATAATGTCTAGAAGGCATGCTGTTTTATTGCC
TGTTTGCATTTTTCACCCAAATTGCTGGAAACTAATGAACATTTT
AAAGGAGCCATTCCTTTAGCTGCAGTCATTTCTCTAAATTGATAT
CTTTTTTTGGGGGGGAGGCAGTGAAATCATTTTAAACATTACTTT
TTGAAAGAAAAAATTATTAGACAAAAGTTAATATTTTAAAATAAA
TCTTGAGAAACTAGAGATTAAATAACTTAATGAAACATATTTCAG
AGTTGAATTTTGGCAGCAATTGTAAATAATATTTATAATCTGGTG
ATATTAAAAGTGAAAAGTTACAAGGGGGAGAAGGAATGCTCTCCT
TTGAAAATTAGTGGCCACATTTGGTGATGGATGAGATGAAGATGC
ATCCTTCCATAATCTTGCCTTTTTAAAAAAATTATAAAGCTCAAA
TGCA

In some embodiments, the intronic domain that promotes trans-splicing primarily in the brain comprises or consists of sequences from the gene FN1. In some embodiments, the sequences from gene FN1 comprise or consist of:

(SEQ ID NO: 75)
GGGGATTTTGGTTTTGGTTTTGGGTTTTGTTTTTTTGCGGTGGGG
GGGCTGGTATTTGGAAGAATTTAGCTCTTTATGTTACAGAAATCT
TTTTTGCAAGGACTTAGAAATGATAATGCTTAAGATTGTTCTTGC
CCAATGTGGGAAGAGAATCTAAGGTTTTTATATGTCTTGCAACCT
CATCAAAGGAAAATTACTGGCATCATTTTCATAATTTGAAAAAAA
AAGCCAAATTAATATATTTCTTTTTTGATTCACTTTTTAAGTGAT
CATTTTTAAAACTTTACTTTTGACCCACTGAATTTATTTAGATAG
AAGGAAAAGAGATGATGGGAGGGAAGTTTAGATAAAGGATGGAAG
TTGGTTTTATTTAAACAATAGCCCTGTGATTTCCTAATGAGAAGT
GACTAGAAATTGAAGAAACCAAATAAGGAGGATATTGGTCAATTT
AGCTTTAGTTTCTCTTACTCTCTCAAGCCTGCCCTGTTTAACTCC
AAAG

In some embodiments, the intronic domain that promotes trans-splicing primarily in the brain comprises or consists of sequences from the gene FN1. In some embodiments, the sequences from gene FN1 comprise or consist of:

(SEQ ID NO: 76)
ATACAATAAGTAAATGAAGTCTGTGTTCTGTATTACATTATTTTT
GTAGGATCGTTTCACAATTTTCTCACAATTCTTACTTTGATAGTA
GAGAAAATAAAAGCAAGCCAAGAGACTTTTTTTTAAAAATATATT
TTATTTGCTATAAACAACTATGATTTGCATTTCTCATGTGAAGAA
TAATAATTATCGGCTAAAAATGGATTTGCTGCATTGATTTTCAAC
GTAAATATTTAAAAGATTAATGCCAGATAATTTTATTATACTCAC
ATTTAACGACAAGCAAAGCTGTTTATAATGATGACTGTCCATGTA
CACAGTTTTAAGTTGAAGTGAGTGAATATTCAAGATAATTAAATG
CTACATTTTCATTTTCAGGCTCAAGTGGTCCTGTCGAAGTATTTA
TCACTGAGACTCCGAGTCAGCCCAACTCCCACCCCATCCAGTGGA
ATGCACCACAGCCATCTCACATTTCCAAGTACATTCTCAGGTGGA
GACC

In some embodiments, the intronic domain that promotes trans-splicing primarily in the brain comprises or consists of sequences from the gene SERPINE2. In some embodiments, the sequences from gene SERPINE2 comprise or consist of:

(SEQ ID NO: 77)
GTGAGTGGCCGCAGTATGTCATTTGATTGCATAGTTTGTGAATGA
AGCATGAGTTAGACTCCAGGGTTCAAAGTGGTGTCTGCAAGTCCA
ATTCAATGGCCTGTGGGTTGAGAAACTCCTGGACCAAGGAAGCTC
TATTCAGTCTCCCTCCTTCCCCTTCAATCTGCCATTGCTCACTGT
GCTCCTGGGAGGTGTTTCTCCTGTAGTTTGGGCAACAGAACAGAA
TCCAGCCCCTCCACTGTCTCCTCTCCTTGCCATATCTGGTCTCCA
GCATCCTTGCCCACAGGCTCTTGTTGATTTTGAAGGTTTTCTTGC
TCATTCCTTCCTGCTTTTGCATCTGCCTCTCCCTTTACCTGGAAT
GTCCTCCTCATCCTTCCAGGTTGATTGATCTCTTTGCCTTCCATG
CCTCTTGCTTCTGCCATTGCCAAAGACATAGGTACCCTGTTGTCC
GTTATGATTGGCACATGATTACAACTTTTACACCCCCCCGATTAC
AGCC

In some embodiments, the intronic domain that promotes trans-splicing primarily in the brain comprises or consists of sequences from the gene SERPINE2. In some embodiments, the sequences from gene SERPINE2 comprise or consist of:

(SEQ ID NO: 78)
GCTTCAGTGGTTAGACTTTAACCCCACTCCCTCCTTTTCTTAAGAA
ATAGGCCCCTGAGAGAACAGAAGACTTAGTTTCTTGCTACTCATA
GCTATCCTGTTTATACTGTTCTGTTATTTTTATTTTTGAGACAGG
GTCTTGCTTAGTCACCCAGGCTGTATGCAGGGGCCCGATCATGGC
TTACTGCAGCCTCGACTTCCCAGGCTCCAGCGATCCTCCCACCTC
AGCCTCCTCAAGTAGTTGGTATCACAGGCGTGCCACCACACCCAG
TTAATTTTGTATTTTTGGTAGAGACAGGGTTTTGTCATATTACCC
AGGTTGTTCTTAAACTCCTGAGCTCAAGCAATCCACCCACCTCAG
CCTCTCAAATTGCTGGGATTACAGGTGTGAGCCACTGCACCTGGC
CTATACTGTTCTTACTTGCTGCAACATAAAAGATTTTAAAAACTT
TTCTAGATTCTTATGGAAGTATTGCTGTATCTTCTGATTTCTCTT
TTA

In some embodiments, the intronic domain that promotes trans-splicing primarily in the brain comprises or consists of sequences from the gene PPP1R7. In some embodiments, the sequences from gene PPP1R7 comprise or consist of:

(SEQ ID NO: 79)
GCCGGCTGCGGTGAAGAGAGCGCAGGGTCCCGAGGCGACCCGAGG
CGCGGGGCCTCGGCGCCCACCGGGCAGGCTACACTGAAGGGCACC
CCGAAGACGCTGGGGGCGCGGGGCCCAGGGCCAACGGCCGCAACC
CCGCCGCGATTTGGGCACGGAGAGGACGCGGGCTCGGCGGTGCGC
GAAGGGCGACGGGCGCAGGGGCGGGAGACCCGGGGTCGGGCTGGT
CTAAGAGTCGGGCGGCGGATCCTCCTGGGCCAGGGGCGCGGAGCC
GCGCAGGCCCCACAGCCGCCCTGGGGAGATGGCGGAGCCCGGCCC
GCTCTCCCGAGCGCAGGTATCTCACCTGGATCAGGCGAGGGTCAC
GCCAAGCCGGCTACACACCACGGAAAGGAGCCCTTCCGCGCTTTT
ATCCCACCGACCAATCGCACAGCGACTAGCACCGATTGACAAGCT
CCACGGACCAGCCACTTGCGCGTATGGGCGGGACTAAGCTAGCCA
GAGT

In some embodiments, the intronic domain that promotes trans-splicing primarily in the brain comprises or consists of sequences from the gene PPP1R7. In some embodiments, the sequences from gene PPP1R7 comprise or consist of:

(SEQ ID NO: 80)
GCCCCCGGGCCGGGCAGATGCGGTTTCCTCCCGACTCGCGATCAA
AATGGGTGAGTAGCGCAGAGCTCGCCTCTTGGAGGCCTCTTTCTG
AAGGCGGAGGACGCGGGGCGGCTCGTTCATGGGCCGGGTGGCTCC
GCAGGGTAGACGAAGGCTGCAGCGTCCGCACTGGGCTGGGAACGA
CTTGCAAGGGGAGCCCAGCTGGCGCCCCGGAGCCAGCCAGCTGGC
TAGCGGCCCCACCAGCGCAAGTGCCCCGCACCTGCCCGCCTGCTC
CGAGCGTCAAGAGAAGGCGGCATTTCGCATGGGTAAAATCCGCGC
GGGGACGCCGGGAGAATGTTGTTGCTCTTGCCGTTCGGCCCATTG
TACAGACGCAGCCAGCGAGGCTCGCAGAGGTCAGGGATGCACGTA
GGACTGGCCCGCACCTTGCAGCCACGGAAAGATCCGCCTGGCCCG
CGGCCCCTCCACGCCTCCGAGGTCGAGACAGTGACATAAGTCAAC
TCTC

In some embodiments, the intronic domain that promotes trans-splicing primarily in the brain comprises or consists of sequences from the gene STK25. In some embodiments, the sequences from gene STK25 comprise or consist of:

(SEQ ID NO: 81)
GTAAGGAAACTGAGGCAGGAGAGGCGAAGCAACTCGCCCAAGGAA
TGGGCTGTGCCGGTAGCTTCTCCAAGGCCTGGTGCGCCCTGGCCT
GTGCGCGTGATCCCGGGCGGGGTCTCAGTACGAACAGGGGCTTCG
CGATGTGGCCAGCGCGGGCTTATCTCCGTGCCGTGACGACAAGGC
TAGAAGCCTGGGCGGCCGCGGTGGATCCTCGTGGCGAGGCGGGGA
GGGCGCCGGGCCTCACTGGCCCGCTCGAACCAGGGCTGCGCAGGG
CAGAGCCCAAGGGAGAGGGCCGCGCGGCCGCCGTAACGTCGGAGA
CGACGCCTGGCAGGCGCCAGCAAACGCAGAAGACCCGCGAGGGCG
AGGCAGGGAGGGCGCGACGCGACAACGACCACGTGCCCCGCCCGC
CCAGGCGCGCGGCCTGCCGGGAGACGCAGTCCAAACCTTTCGGGA
GCAGGACTAGGGGTCCCAGAGAGCCTCGCGGCGCGCATGCGAGCC
CCGG

In some embodiments, the intronic domain that promotes trans-splicing primarily in the brain comprises or consists of sequences from the gene STK25. In some embodiments, the sequences from gene STK25 comprise or consist of:

(SEQ ID NO: 82)
TTTCACCATGTTGGCTAGGCTGGCCCCGAACTCCCAACCTCAAGT
GATCTGCCTGCCTCAGCCTCCCAAAGTGCTGGGATTACAGGTGTG
AGCCACTGCTCCCAGCCTCTTCTAGGCACTTCGGAAAAGTGCAGC
GATTACTGACCACACGAAGGCCAGAGATGCAGCAGGCATCTATTT
ACATGCATTAAATACATATATTATCTTCATGCATTTACACATTCA
TTCAACAAACATTACTGAGCGCCTGCCGTGGGCTGGCATGGTCCT
GGGCACAGCTCCGCTTGCAGGAAGCCCACTTTCTCCTGGGGAGTC
AAAGCACAAACAAAGCCAAGTCAGTACGTAAAGAAGCAGGGGTGA
TGAGAGATGCGGACCCAGGAGGGGTTCAAGGGGCTGTCCTGGACG
CATGACAGGGAAGCATAGCTTCTTAAAGGACTCGGCAGTGCACCA
TGAGAAGACACGCACCAGGGAGCAGAGAGAAACGCCAGAACCCTC
TCGA

In some embodiments, the intronic domain that promotes trans-splicing primarily in the brain comprises or consists of sequences from the gene MOBP. In some embodiments, the sequences from gene MOBP comprise or consist of:

(SEQ ID NO: 83)
GTTCAATGGTATGTCTGTGAGATCAAAATGTGTTGATGTATGTCA
GCAGGTCAGCAGTTTGTTCTTTTTCCTTGTGTAGTATTTTATTGC
ATGAATGATGATTGTTTTAATAATAAATAATAATAAATTTTTTAT
TTTGCTTTTTAGAGAAAAGGCTTTTCCTAACTCTAGAATATTATT
TAGTCAAGGATATGTTTGGTTTTAAATGTTTTCCTATCTTACATG
GGTCTCTAAAACATAAGGAAAACAAGAGCCCATGTTTCTTTTTCT
CCTTAAAAACACGATTTTTAAAAATACACAAATGAAAATTAATGA
ACTGTTAGTTGAGTTCCTTTCCACAGAGTTGGGGGGAAGTTGGTC
TTCCAGGGTGTTGCTCTGTAGGGCCCCCCTGAATGTTACCTTGGA
TTATGGGAGTAGCAGGGGGCTTCCAGAGTAGAGGGCTCCCTTTCC
TGATGTGCGTTTATGTCTCCTCCTGTCTCCTTGCATCGGCGATTT
CCAG

In some embodiments, the intronic domain that promotes trans-splicing primarily in the brain comprises or consists of sequences from the gene MOBP. In some embodiments, the sequences from gene MOBP comprise or consist of:

(SEQ ID NO: 84)
TAAGCGGCCGCCCAGCCCCGCGGCACCAGTTGGGCACAGCGCGTG
GTCTCGGCTCCCAGCACGCCCCTCCCGCTCCGCACCCCACTCTTC
CCCCTAGTCGGCTCCGGGTTAGGCTCCGACACCGGAAGGCACTCC
AGGGAGACTGGAAGGTGGGTGGGGGAGCAGGGCCTTCCTACCTGT
TTCCAGCTCCTGCCAGCGTCGCTTAAGCAGCAGAGGAGAGCCCTG
GCTCCCGCCTCCAGCTTCTTTTGGCCCTCTCAGAACCAGCCGCCG
TGCCAAGTCCCCTCAGAGGCCCAAGCAACAGCCAGCTGCGCCCCC
CGCGGTGGTCAGAGCGCCAGCCAAGCCACGGTCCCCTCCGAGGTC
TGAGCGTCAGCCACGGTCCCCTCCGAGGTCTGAGCGTCAGCCACG
GTCCCCTCCGAGGTCTGAGCGTCAGCCACGGTCCCCTCCGAGGTC
TGAGCGTCAGCCACGTCCCCGCCCAGAGGTCCGACCACCGCCAGC
CAAG

In some embodiments, the intronic domain that promotes trans-splicing primarily in the brain comprises or consists of sequences from the gene HHATL. In some embodiments, the sequences from gene HHATL comprise or consist of:

(SEQ ID NO: 85)
GCAGGGACTGGGATGTGACAGGCTGGAGCTCAGGGACTTGTTGGG
GGAATGGGGGTCAGTGACAGGCTGGAGGCTTAATGCGAGGATGGA
GGCTTGGCTCTAGGGGCAGCACCTGGGGTGGTCATGGGATCTCAG
GGAGGGGAGTGGGAGTCAGAGAAAGGGTCTTAGAGAAGCTGGGGC
TTTGTGAGGAGCTAAGGGAGACTCACTGATCGAGGTGGGGACCCT
GCAGGCAGAAACCTGACTGTAGTGATAGAGGTGGGGTTTCTCCGC
ACGCAGGTCATGGGGGCCTTGGCAGGAGCGGTTTTGGCAGAGGGT
GGGGCCGGTGCCTCTGGAAGGTATGAAGATGTAAATGAAGGTGAG
GGAGGCAGGCTGCGAGTGAGGGCTCTGCGGGCAAGAGCCTTGATG
GGTGGGGTGGGGGCAAGAGCAGCAGCTGGGATGGGGCACTGGTGT
TCGGTGGCCTGTGAACTGTGGGGGGCACAGGGGCAGGCAGAGTGA
TGGT

In some embodiments, the intronic domain that promotes trans-splicing primarily in the brain comprises or consists of sequences from the gene HHATL. In some embodiments, the sequences from gene HHATL comprise or consist of:

(SEQ ID NO: 86)
ACCTTGACAGGTCCCTGCCCCTATCTGGCCCATGACCCCTCCATC
TGTACGAGGAGACCGTCGGCTGCTGGGGGGCCCAGGGGAGATGGC
TACAGGGCTGGGGATGAAGATTTAGGGGAGGATCAAGGGTGGGCA
AAAGGGCCACATCAGCCACCTCTTGGCCCCGTCAGCCCAAGAGTT
GCCTGGACATGTTCCCTGCTTCCTCCCTTTAATTTGGGTCCTATG
TCTGTATCCATGGAGTCCTTGGGAAGCCCCTGAGCCAGGCTGAAG
CGGGATAGGAAGGGTAAAGGTGCTAGCTGAGCCGTAGTACGGCCC
AGCAGTTCTCCAGGGGACAAGCTAGGCCCCCGCTGGAGGGAGTGG
TGAGACTCAGCCGGGGCCTGATTCTGGTCAGCACCTTGGACAGCA
GCCAGTGCCCTCCCTGGCCCCGCCTGGCCCCGCCTGGCCCCACAT
CCGCTACTCCTGGGCACCTCCTCAAATAGCACAGCCTCCAGCTGG
TGCC

In some embodiments, the intronic domain that promotes trans-splicing primarily in the brain comprises or consists of sequences from the gene SHISA5. In some embodiments, the sequences from gene SHISA5 comprise or consist of:

(SEQ ID NO: 87)
GAGGAGCAGCCGCGCCCTACCCCGCCAGCCAGCCTCCTTACAACC
CGGCCTACATGGATGCCCCGAAGGCGGCCCTCTGAGCATTCCCTG
GCCTCTCTGGCTGCCACTTGGTTATGTTGTGTGTGTGCGTGAGTG
GTGTGCAGGCGCGGTTCCTTACGCCCCATGTGTGCTGTGTGTGTC
CAGGCACGGTTCCTTACGCCCCATGTGTGCTGTGTGTGTCCTGCC
TGTATATGTGGCTTCCTCTGATGCTGACAAGGTGGGGAACAATCC
TTGCCAGAGTGGGCTGGGACCAGACTTTGTTCTCTTCCTCACCTG
AAATTATGCTTCCTAAAATCTCAAGCCAAACTCAAAGAATGGGGT
GGTGGGGGGCACCCTGTGAGGTGGCCCCTGAGAGGTGGGGGCCTC
TCCAGGGCACATCTGGAGTTCTTCTCCAGCTTACCCTAGGGTGAC
CAAGTAGGGCCTGTCACACCAGGGTGGCGCAGCTTTCTGTGTGAT
GCAG

In some embodiments, the intronic domain that promotes trans-splicing primarily in the brain comprises or consists of sequences from the gene SHISA5. In some embodiments, the sequences from gene SHISA5 comprise or consist of:

(SEQ ID NO: 88)
GTCTAGCTCTTTGGCAGGTTCGGAGCGACCTTGGCCGTTGGCCTG
ACCATCTTTGTGCTGTCTGTCGTCACTATCATCATCTGCTTCACC
TGCTCCTGCTGCTGCCTTTACAAGACGTGCCGCCGACCACGTCGT
AAGCGTGCCCACCCTGCCCCACTGTGACCCCCTGGGGTGGTAAGG
GAAGCTGACCAGGCCCTGCTTTGGCTAAGCTGGAGGGATGGGTGG
CAAGCCTGGCTCTCAAGACCTGGGGAGATGGAGGAGGGTCCTGAC
TGGAATTCTCTTTGCAGCGGTTGTCACCACCACCACATCCACCAC
TGTGGTGCATGCCCCTTATCCTCAGCCTCCAAGTGTGCCGCCCAG
CTACCCTGGACCAAGCTACCAGGGCTACCACACCATGCCGCCTCA
GCCAGGGATGCCAGCAGCACCCTACCCAATGCAGTACCCACCACC
TTACCCAGCCCAGCCCATGGGCCCACCGGCCTACCACGAGACCCT
GGCT

In some embodiments, the intronic domain that promotes trans-splicing primarily in the brain comprises or consists of sequences from the gene GNAI2. In some embodiments, the sequences from gene GNAI2 comprise or consist of:

(SEQ ID NO: 89)
ACACAGGATTAGCCAGCGACCTAATCCGCCAAGCTATGCAAAAGG
AGGCCTCTGCTGACCCCTGGCGGTCACCAGCCGGAGTCTGGCTGC
GCGGTGACCCACCACACGTTGCCCAGCCGTGTGGCTGGGATGGTC
GGTACCCATGGCCGTGCGGTCGAGCCTTGATGCTCCAGGACAGTC
CTGGAAGGCTTCCTGGCAGATCCGTGGGAAGAAAGCCGACAGAAG
GGCGGGAGGCGGGAGGGGAAAGGTCCCCCGGGATGGGTAGGGCCT
CAACGCTGGGCATCGGCGCAGGTGTGTACTGCAGGGAGCCAGCCA
GACCCCCCACGGACTGCCTGACCTTGGGGGAAGCCCGGAGTAGAG
GAAGAGGCCGGACCCCCAGACCCGCCATTGACTATGTCCGCCATT
GACTATGTGTTCTGAGAGAAATTTTTTTTTTTTTTTTTGAGACTG
AGTCTCACTCTTTCGCCCAGGCCGGAGTGCAGTGGCGCGATCTCG
GCTC

In some embodiments, the intronic domain that promotes trans-splicing primarily in the brain comprises or consists of sequences from the gene GNAI2. In some embodiments, the sequences from gene GNAI2 comprise or consist of:

(SEQ ID NO: 90)
TAAGCCGATGGCGGCTATCGCGGAGGAGACCCTGGCGAGGTGGGG
CCCCGCGCGGGGCAAGGGGGATGGGGTGCCACAGAGGGCTAGTTG
CAAGAGGACGCGTGACGGCTGCAGCTCTGCGGAGACCGGGCCTCG
ACGGCCAAGGAGTGGCAGGTCGGCGGAGGCGGGGGCCAGGCCCGG
AGGCAGAGCGAGACAGGAGAGCAGGCAAGGCGGGGGCCACCGTGA
GTGGGGGCCGCGAGGCGGGGGCTGGGGCGCGGGGAGGTGCTAGGT
CGGCCTCGGCTCCCGCGCCGCACCCCAGGCCCGCCAGGAGGCGCC
GCTGCCCCGCGAAGCCGTTCCGGCCTCTCCCGGCCACCGAAATCC
CGCGCTCCGGCCTAGAGAGGCCCCGGAAGAGGCGGTGCAGCGCAC
CCCGAGGCCGCTTCCGGCTTCGGCTCTGCAGGAGAGGCCAGAATC
GGGGGAGGACGCCTTGGGCGGGGCGTGGGTGAGAGAACCTGGGGG
AGGT

In some embodiments, the intronic domain that promotes trans-splicing primarily in the brain comprises or consists of sequences from the gene TKT. In some embodiments, the sequences from gene TKT comprise or consist of:

(SEQ ID NO: 91)
GGCAATTGCCCCCCACTTAGCTACCCAGCCAGGTCTAGCAGTGCC
AGAGGCTGTGCCTGTGTTTGCCTCAACACCAATTCCAGAGGGACC
AGGCATGGGGTGATGTGGTTTGGTCCCAGGGGGGTGCCCTGGGCT
GATGCCTGTTCACCAGTGTGTTGAGGGCAGACCTGGGATTGGAAC
CTGGGTCTCTGTGAGTCTAGAGATCCCACCTTCTGTACCCTACTG
CCCCTAGGGCCCTCAAAAGAGGAATCATGCAGTAGGCTCCCAGGA
GGGCCCTGGTCCCATAGGATGATGGGAACAGCCTGGGGCTCAGGT
CCTGTGCGTCTCCAGAGCCCACAGGCCCCTGCAGAGAATCTAGAG
GCAAATACTGGGACAAGGACACCTGAGCCATAGGCACAGGCCCAG
CCAGCAGCCTGGTGAGCCCATCTACACTGGCTAGCCATGCCCCAA
GGACCTGGGAGGCCTGGCTAACCCAAAGTAGAGGGACAGGAGGGG
CAGC

In some embodiments, the intronic domain that promotes trans-splicing primarily in the brain comprises or consists of sequences from the gene TKT. In some embodiments, the sequences from gene TKT comprise or consist of:

(SEQ ID NO: 92)
TGTCTGTCTCTTTAAAATAAAAAAAATCAATGAGTTATGTGATGG
GCTCATGGCTACAGGTGGAGAAAGGCAGTGCATATGCAGCCTCCT
CCATCCTTGACTAAGGCTGACAGAGGGCTGGGCCCACCACTGCTC
ACCCTGAGGCCTCGTCTTCTGACTCCCCTCCTTTCATTTCTAGGT
GGCATTGGTGAGGCTGTGTCCAGTGCAGTAGTGGGCGAGCCTGGC
ATCACTGTCACCCACCTGGCAGTTAACCGGGTACCAAGAAGTGGG
AAGCCGGCTGAGCTGCTGAAGATGTTTGGTATCGACAGGGATGCC
ATTGCACAAGCTGTGAGGGGCCTCATCACCAAGGCCTAGGGCGGG
TATGAAGTGTGGGGCGGGGGTCTATACATTCCTGAGATTCTGGGA
AAGGTGCTCAAAGATGTACTGAGAGGAGGGGTAAATATATGTTTT
GAGAAAAATGAATTGGCCCTGAAATGTTTTCTTTTTTGATGTGTT
TGCA

In some embodiments, the intronic domain that promotes trans-splicing primarily in the brain comprises or consists of sequences from the gene CLDND1. In some embodiments, the sequences from gene CLDND1 comprise or consist of:

(SEQ ID NO: 93)
GTATGTGAGCATTCAATAAGTACTTTTATGAGAATGAGTTTTGTT
TTAAGCTTAATTAAAAACATGCTTGCTTTCTAAAAAAAATTCTTA
ATGGTTATATGTACGAAGGGATGGGGAATTGGGTAAAGGGTGCAT
TTGGAATAATTCAAAATAATCTTTCTTATTTGTCCGTAAACTCTA
ACTTATCTTCTTTCTTCAACTTCTAAACAGAGCAGTCTGAATGCC
AGAATGGATAACCGTTTTGCTACAGCATTTGTAATTGCTTGTGTG
CTTAGCCTCATTTCCACCATCTACATGGCAGCCTCCATTGGCACA
GACTTCTGGTATGAATATCGAAGTCCAGTTCAAGAAAATTCCAGT
GATTTGAATAAAAGCATCTGGGATGAATTCATTAGTGATGAGGCA
GATGAAAAGACTTATAATGATGCACTTTTTCGATACAATGGCACA
GTGGGATTGTGGAGACGGTGTATCACCATACCCAAAAACATGCAT
TGGT

In some embodiments, the intronic domain that promotes trans-splicing primarily in the brain comprises or consists of sequences from the gene CLDND1. In some embodiments, the sequences from gene CLDND1 comprise or consist of:

(SEQ ID NO: 94)
TATGTTGCACTGAGGATTTAGGGAGAGAGCAAGATTTCTGTTGAG
GAGGTTCAGAAGTATAAGGAATCGGGAGAGTAGATAGGAGTGAAG
CATTTGGCAGCGTAAAGAAGCTTGGCTTAGCTTTAATGGATTAGG
GAGAGATTGCTATCTAGCATTATTACATGTTATTGTTCTCTTTGC
TATTTTATACACCTAGAACAACCTTTCCCTCGGGGTTAACAAACA
TCTTCAACTCTGTTTTCTGTATGGCTCCTGACTCTTACCTTTGTT
TTAGGAGGCTTCCCTCTTCAGAGGAAACTGGGAGAAGCCAATTTC
CACTGCTTCTTTTTCCTGTGCCTTTTTAACACTCAAACCCAGAAC
ACTTAATCCTCTGCAGCTCGTGCAAAAATTCAGTCTGTAATTTAA
AATGTGTGCACGGTACACTGCTGACCATGGGCATGTCTATTGTTT
GTATCTGTAATGTGTATGTCCATTTCAGTGTTGTCTGTTTTAGTA
TGCA

In some embodiments, the intronic domain that promotes trans-splicing primarily in the brain comprises or consists of sequences from the gene CD200. In some embodiments, the sequences from gene CD200 comprise or consist of:

(SEQ ID NO: 95)
TGGAGGCAGACACCTCTGTTCCACCTGCTTCCTTTGTAGATTGTG
TATGTGTGTGTGTTTGTGTGGAATGCATAGGGCTGCTCCATCTTC
TTTACTTACAGTGGTCACTCTGCCTGTTATCCTTTGGCCTTTTCC
TGCTCTTTGCTTCTACTTCTCCAAGCATGGACAAGATTGGTGAAG
CTGCTATCCTACCTGGCAGTTTACATCTTCAATCTCATTGTACCT
ACTTCGCAGCTTACACAGATCATTCACAATTTCTTGTAACTGAGA
GTTGCTCTCTTGTTTTTCTACTCTGCTACTATTCTTTAAAGATTG
AACAAACATATTTGAACCTATATATTTCTCTGGCATCACGTAGGA
ATGTAAATAAATGTTTTCTTTTTTGCATTTTCTCTTTCTTTTCTA
TATTTGACATTGAATATACATTTTATTTATAATCAGGAAAAAGTG
TATGTGTGTTACAATCTTTAAATATAAATGTTCTTTTATGATTCC
ATAG

In some embodiments, the intronic domain that promotes trans-splicing primarily in the brain comprises or consists of sequences from the gene CD200. In some embodiments, the sequences from gene CD200 comprise or consist of:

(SEQ ID NO: 96)
TGAGAATCTCTGAGAATCATTGTCTGTGTCTGGAAATACTATTTG
CAAGAATGTTTGGAATATAGCCGTAGTGCCCAGTTTTTCAGGATT
TTAACCACAGAAAGGGTCATGAGAAGATAGCCTTTCTTGTCTACT
ATAGCTGTGTTTATGATTATTTGGAGAGTTCATGGGGCTACACTG
AGTTCTTTGGCTGGAGCTATCTTTTCTCTGAGGGATCCTGCTGTC
AACAGGAGAAATTTGAGGACAATGGTGAGGGCAACAAGTTCTCCA
TAAGCAGTGATATGATTCAGACCTGGCTTTGCCACTAGCAATCAT
GCGACCCTAGAAGGGTCCTATCCACTCTCCACTGGAGTTTCATTG
TCTGGACAATAAGTGTGAAATAGATCATTTCTAGGGTCACTTACA
ACTTCAACTTTTTGGATCTAAGGACAGAGCCAGATTGGACTAGCC
TTGAGCCAGTTATGATTGTCCTTCTCATTTTGCTTGGGGAGATAG
CTCT

In some embodiments, the intronic domain that promotes trans-splicing primarily in the brain comprises or consists of sequences from the gene MCM2. In some embodiments, the sequences from gene MCM2 comprise or consist of:

(SEQ ID NO: 97)
TGGGAGCCCACGGGGTCCCCAGGAGCCGATCTGAGGAAGTCATTG
TGCAGCAGAGGGTTCCCCTCTTCTGCATATCCTGCCAGAGTGGGG
AACAGTGAAAGTGGGACCTGGGACACCTGGGTTTCCTGTTGAGTC
ATGTTCCTGGAATCACCTTGGATGTTCTCAGAAGGCATGGGAGGA
GAGCCCAGTGCCCCTCTGGCCAGGATGGAGTTGGCTGTTGGTCTC
AGCAGCGAATGCGTAAAAGAACCCAGGCTCTGTCACCCACTGTGT
TCTTGGTTTTTCCTCCTGGGGAAGCAGTTATGCTTTCTGTCTAAA
TTAGGCTATTTTGGGCCCTGGTTAGGGTAAGCTCTTTGCTGTCTC
AGACAGCAGGTGCTAAGTACCCACCTATGTCTTCCTCTTCCACTG
CCCTCATCCCCTTCTGCCATCAGAGCAGCCTGGGGTCCCTGAGAT
GGGAGGATAGGGGAATCCACCCTGATGGAGGTGCTCCCCTGTGTT
TCAG

In some embodiments, the intronic domain that promotes trans-splicing primarily in the brain comprises or consists of sequences from the gene MCM2. In some embodiments, the sequences from gene MCM2 comprise or consist of:

(SEQ ID NO: 98)
TATAGCTCACATGTGCCCGGTTTCCATGAACTGTGGTTTGGGGAC
CTCAGGTGAGGCTTGGGGTCATACAGTGTGCCCAACACAGGGGAC
AGGTGCTGCAGGGGCCATAGGCTGTTTTCTAGTCCTGTTCCCTCG
GTCTCTTCTCATGTCCAGAAGTCGCCCTGATTTCTCTTTCCTTCA
CAGCACCCCCGACCCCCACCCTACATAACCCACAATCCACCAAAT
CCTGTTGTCCCAACTCCTGAGCCTACCCACCTCCCTCACTTCTGC
CTCTGACTTGAGGCACTACCATCATCTGAACTGGCCTCCTGGCAC
CCATGTAGACTGTCTTCCAGTTGAAGGCATAAATCAGATCAGCCC
CGTCCTTAACAACTGCTTGAGGCTTTCTGCTGTCATCAGAATAGA
ATCCAGGGTCCTCCCCTCCGGCCCGCACCCGCCATCTCTGCAGCT
ACCTCCTCCTCGCTCCCTTCCCTCACCTGCACCTGCACCTGCCCT
TCTC

In some embodiments, the intronic domain that promotes trans-splicing primarily in the brain comprises or consists of sequences from the gene TMEM108. In some embodiments, the sequences from gene TMEM108 comprise or consist of:

(SEQ ID NO: 99)
AATGGGATAAACCTGGTTTTCTGAGTGGAGGCAGCTATTGATAAT
TTTACTTTTCTTATAGCTGAAGATGAAGGAGATTTGAGGTTCTCT
GAGAATTAAAATTCGGCTGGAGTTAGCCTAGGGTGAGACCCTCAG
AGCTGTCTGTTCATGCCATTGCCTCAAGCCTGTGGACGTTTTTTC
ACCCTGGGGCAAGAAGAGGCTCCAGTTTGCCCAACTGCTGTGTGT
GTTCCAGGCCAGTGTCCAGTGAGGCTGACTTGGCAGCTGCCCTTT
CCACAGCCAGCCAGCCAGCCCCTGAGTGTGTTTTAAAAGATAAGC
GACAAAAAGCCAACCCCTTTTAGAGGGGCTGGAACAGGCTAGTAG
TTGCTAGGCAAGTTAAAGTAATTCAGCGTCCTTGCCAAACGTCTG
CCAGGCAAATAGGGAACTTTCAGAGAAGGGGAGTGAGATGGTGGT
GTGTGTTGGTTGCCATAGGAATGTTTCCCTGATTGGAACAAGACC
ACAG

In some embodiments, the intronic domain that promotes trans-splicing primarily in the brain comprises or consists of sequences from the gene TMEM108. In some embodiments, the sequences from gene TMEM108 comprise or consist of:

(SEQ ID NO: 100)
TAGGTATTTTGTATTTATGTAAATATTATGTCAGATTATGTCTTT
ATTTTAGCTCTTAAGGTAGTAATTAAATATCTGTTCATGAATTAT
AGTTTTTATCGAGTTCTGATTTATAGGGGGAAAAGGCACAGTTGA
CCAAATTGTATGAAACCTTGGACCTTGAAGAGACCCTCTGATGTG
GGCTTTACTGCCGCATTAACAAGCACCCTGTGCATCTGGTGGCAG
CTTCCAGAATGACAGCTGAGGGTCTGAGTGGAAATAAGTGGCTTT
TAGCAAATGTTGAACCTCGAATGTGCTATTTAACCTCCCTGATTA
ATTGATTATCCAGCTCCGAAAATAAGAGGTGATATAGCTATCCCC
TTATCTCAGCATGGATGCTGTTCTCATTTGAATACAATTTGGGGG
GTAGGTGGGGGTAGTATCAGATCCTAGGGTACATGCTATGATAAT
GACAGGCTTAATTATCAGTCAGTCAATATTGTTTAGAACAAATGT
TTTC

In some embodiments, the intronic domain that promotes trans-splicing primarily in the brain comprises or consists of sequences from the gene EIF4G1. In some embodiments, the sequences from gene EIF4G1 comprise or consist of:

(SEQ ID NO: 101)
GCCAGGTGAGGGGCTGTGGGGAGGGGAGTAGGGGACCTGGGTGGG
AGCAGTGGAAAGCTTTGGCCAGTTTAGGTCTAGGATGGAAACTGG
AGGCCCCATACCTGGCCTTAATCCTCTGTGTTCTTTCATGCGGCT
CTGCGCCTTGCCCTGTTGATTACTCACGGTGGCTGGTCCCTTGGA
TAGTATGTTGTCAATGAGGGTGGCATTACTGGGATCCTAGTGCCT
CTGAGAATTGGGGGCAGTGCTTTGGGCCCTGCTGCCAGCTCTTGC
TTTCCTATTTGCTCCTTTTACCGGAGGCTGCTATTGCTCTATTGC
TCTCTCTGCTTTCTCTTTGGACTGTGGTGAGGTAAACACTCCAGC
TGGTCCTTGCCTTTCTCTGTTCCCCAGCTTCTTCCTTACTAAGTG
GGAGGATTCTTGTCCTGTCTTATTGCCTTCATTCCCTCCCCCCGC
CATCACCTTGGGGGTAATTCTCTTCTTTCCTATCCCCATGTGCTC
TCAG

In some embodiments, the intronic domain that promotes trans-splicing primarily in the brain comprises or consists of sequences from the gene EIF4G1. In some embodiments, the sequences from gene EIF4G1 comprise or consist of:

(SEQ ID NO: 102)
TAATATCCCTTTCCCAACCCTGGACAGTCTTCTGGTCTCATCCTT
ACCCTCCACCCTAGTCAGGGGCTAGACACAGGGAATGGGGATCTT
CAGGGTAGTGACTTGTTTGGTGTCCAGGTTCTCAGTTGGAGGCCG
AGTGATGCATGTTAGGGGCTACATCTTGAAGAACGCGCATCTGGG
GACTGGGATGCTGTTGCATGTTATGGTTAGCTCCTCATTACATGC
TGGTGGAGCAGTGCATGCTGGGGATATGTTGCCCGAAAGGGCCTT
ACTTAGGCATGATGCATCTCGGTGTTAGGAAGCCTCTGCAGAGGG
GTAGTTTGGTAGAGGTGACGTGTGTATGCATGGCAGTGAGAGAGT
AAATCTCTGCAGGATGCAAAGTGTACATCTGAATTTGGAGGTTTG
GAGGGCATGTGTGTGTACAAGAAGCCGCTTAGCCATTTGGTCAAG
TGGTCTTGGAAGGATTGGTCGCCATGTTCTGAAGCCCACAGTACT
TCTT

In some embodiments, the intronic domain that promotes trans-splicing primarily in the brain comprises or consists of sequences from the gene TACC3. In some embodiments, the sequences from gene TACC3 comprise or consist of:

(SEQ ID NO: 103)
CTATGTCTAGCAGCAGCCTCTGTAGGTGTCTGGCAGGACGGGAAC
ACCAGGCCTCATCTGGCCTCCCAAAAGCTTGGGCTCCTGGAGGTG
CTACTGTGGGGATCTGTCTTAGGCTGAGTGTCAGCCGGCGGCAGG
TGACCGCCTGGCCCTTGCAGTCCCTCCAGGACCACCACGAAGCAC
GACGCACATCCTGGAGCTCGCCATACCGGCTCCCAGGCCTCACCC
TAGGCTCTAGGGTGTTGGGCGCCGCCTCTTCAGGGACCTCTGGCC
CAGGCTCCAGTTCCCTGCGGATCTGATGTGGAGGAAACAGCTGTT
TCCTGGGCTTTGCATCCGGCCTAGAAGATCCTGGGTGGAGGGACC
CATCACCCTCCGAGGCTCACACCCACTGCCCACCTCTCCTCCTGG
CGCTCGAGTCCCTTGCCTCATTCCTTGGGCTGTCTCTGGCACCCA
TCGTTTCGGTTGCCTCCTCATCCTGAACGTTTGCTTTTCTTTTCT
CAAG

In some embodiments, the intronic domain that promotes trans-splicing primarily in the brain comprises or consists of sequences from the gene TACC3. In some embodiments, the sequences from gene TACC3 comprise or consist of:

(SEQ ID NO: 104)
TATGTCTCCGCCACCCTGGTGTCCTCACCTCGGAGGCTGATGGAC
TCATGGTCCAAGCCAGCGGGGCTACAAGCTGCTGTCTTTGCACCT
TGGTCCTTGGCCAAGCCTCCCCTTGCCACGGGGGCATGGGATGAC
CTGTGAACGAGACAGGGTGCAGGAGTCACAGCATAGCTGGTTTGC
GAGGCAGCTGAGGGCGGCAGGAGGCAGAGTGAGCAGGGAGGCCAT
CTGAGCCGTCCGCTGTTGTGTGACAGACGACTCGGCTATGATCAG
GCATCTGTGAGATTTGAGACAGCGCTGGTGCACCGGGGCCTGGGG
CACCTCGGAAAGGTGTCTGGGCTGTCCGAGCGCTTGGGTCCCAAG
GGTTGGGGACCCAGGGCATGCAGTATCCAAGGCGGGAGGCCCACT
GAAAGGTGGTCACGGCTGTGCAAGGCCACCCTGGTGTCCTCCTGG
CCACGGGACTCTGAGCGCCCCCCACCTTCTGTGCTGGGCAAGGCC
CACA

In some embodiments, the intronic domain that promotes trans-splicing primarily in the brain comprises or consists of sequences from the gene ADD1. In some embodiments, the sequences from gene ADD1 comprise or consist of:

(SEQ ID NO: 105)
GCCGGCCGGGGTGTACTCCGCGCGGGACCCTGGGCCTCCGCAGGC
CCCGGCGGGGTCGCCCCGAGGCGGGCCTGGCGAGTCCAATCGCCC
TGCCCGCAGGGGACCCGCCCCTCATGGGTCCCGCCAATCGCAGGG
TACGTTGCTCGGCAGTGCTGCGCGGCTTGGCCTATGGGACGGCGC
GTTGTTGGGCTGCAGCGCCGCAGGCCGCACCCAGGTCGGGCGGTG
GGGGCGAGCGGAGGGGCTGAGGGGCGGAGAGGCCTGGCGGGCCGC
TGCTGCGGGCCAGGGGACGGGGGCGGAGCCGGAGCCGGAGCCGAC
GGGCGGTGGCCGCACTGGGACCCCGGAATCCCGCGCGCTGCCCAC
GATTCGCTTCTGAGGTGAGGCTAGCTAGCGGGGGCGGCGGGGGCC
CGGTTCGGGCTGAGGCGGCGGCGGGGGCGCGGGGAACTGGGCCGC
GGTTGCGGCGGGCGGGGGCCGCGGGGCCTGCGGCGGCCCGGGCAG
CCTC

In some embodiments, the intronic domain that promotes trans-splicing primarily in the brain comprises or consists of sequences from the gene ADD1. In some embodiments, the sequences from gene ADD1 comprise or consist of:

(SEQ ID NO: 106)
TAAGTTAAAGAAAGATTTTTTGTGTGAAAAATTGACTGAGATCTT
AATGGGGATAGGAAATAGAATGCTGATAGGTCGACTGTGTCTATG
AGTAGAGGCGCCTAGTGAAAACAAGCTTGGAGTGGATGAGTAGGA
GTTCAACAGGCACCAGAGAGAGGAGGGCATTTCAGGCAGAGGGTG
TGAATTGTATGAAAGTGAGTTTGTAAACTTTGGAGCCTTGAATGG
ACAAAGGCCCGCGATGATACCTTTTTTTTTTGAGACGGAGTCTCG
CTCTGTCGCCAGGCTGGAGTACAGTGGCGCGATCTCCAGTCACTG
CAATCTCCGCCTCCCAGGTTCAAGCAATTCTCCTACCTCAGCCTC
CCGAGTAGCTGGGACTACAGGCGCGCGCCACCACGCCCAGCTAAT
TTTTGTATTTTTAGTAGAGACGGGGTTTCACCATGTTGGCCAGGA
TGGTCTCAGTCTCTTGACCTCGTGATCCGTCCGCCTAGGCCTCCA
AAAG

In some embodiments, the intronic domain that promotes trans-splicing primarily in the brain comprises or consists of sequences from the gene UCHL1. In some embodiments, the sequences from gene UCHL1 comprise or consist of:

(SEQ ID NO: 107)
CCTCCCGAGTAGCTGGGATTACAAGTACACACCACCACACCTACT
AATTTTTATATTTTTTGTAGAGATGGGGTTTCACCATGTTGTTCA
GGCTGGTCTTGAACTCTTGTGAGCTCAAGTGATCTGCCTGCCTCA
GCCTCCCAAAGTGCTAGTATTGCAGGTGTGAGCCACCGTGCCTAG
CCAGATCTCAGTGTCTTTATAACTACAACTGGCTGCCACTTTCTA
GCATTGACCTTGGTTGAGATAGTTAGCCTCTCTAAGCCTCAATTT
TCTCCTCAGTAAAAATCCTGTAGTAATAGCATCCTCATAGAGTTG
TTCTGACAGTTAACAGTATATTTACCTTAGTGGGCTTAGAATAGG
GCTTAATGTAAGACATACATTAAATATTAGCTATAATTTCTAAAA
ATCAAGTCAGAAAAATTTCTTGACTTTCTTTAG

In some embodiments, the intronic domain that promotes trans-splicing primarily in the brain comprises or consists of sequences from the gene UCHL1. In some embodiments, the sequences from gene UCHL1 comprise or consist of:

(SEQ ID NO: 108)
TATGTTTTACTCCATTTTTGGAACCCAGTGTAGTTTCATGTGTTC
TTTCAGACTGAATTTCTCTTGATATATTGTGTGACTTTATGGCAC
TTGGCATATCATTGTTTATAAAGCCACAATAACAAAGTATTCTCA
TGAGGGCACTTAACCCCTTATCATCCCCAGAGCAACTTCCTCAGG
CAACTGACTAGCATCCCCTAGTATCATCCTTTCTGGGCAAAGAGT
ACGTCTGAAAAAGGATCAGTGCCTGAATATGACCTTACTTGTCTA
AAAAGAATCTGAACATGTTGGAATAACTTCTATTAACTAGTTTAT
GGCATCAAGAAAGACGACCCACCTTGTAAGATTCATTCCTTTTGT
ATTAATGGAGAAAGACTTGTAACTCTTGTCTCAAGCCAAGATACG
ACCTACAGGCTTACAAAGGCAAAGGAATAGTGAAGGCAATCTACA
GTCATTGGAGGGCAGATAAAATCTCAGGCGTCTCCTCCTCTCGCC
TCCA

In some embodiments, the intronic domain that promotes trans-splicing primarily in the brain comprises or consists of sequences from the gene OCIAD1. In some embodiments, the sequences from gene OCIAD1 comprise or consist of:

(SEQ ID NO: 109)
ATTCCTAGGCTGCTGGCTCTCAGACCCGCAGTGAGGTCTGACGTC
ATTTCCTGCCGTAAGTATACAGTGCCTCCGGGTCGCGGTCATTTT
GAGCCCCTGTCTGGATGACTTCTTGCGGCTGTTCTACCCCTCCCC
CTCCCCGCGGTACCTTGCACTTTTCTCCCTCCCTGCCCCCTCTCG
AGTCCACCCTCCGGGCCTTCTGCCCCTGATCGCTTGGTTTTCCTT
GCAGTCGCCTGCTGCTGTCGTCGGGAGGTGGGTGAGGTGACGCAA
ACAGCCCCGTTGTTGCCCTCCGCGTATCCCCTCACCACCTTTGCG
GCCATCCACGACTTTCGCACCTTCCGCCATTTTCCTGCCTGTGAG
GGTGGACAGATCGCGCTCGGGTCTCGGCCTCCTGAGTGCCGGTGA
CTGCGGGAGGCGACGGAGTGCTTCTGGGGGTGTGAGCTGGGGAAG
TTCGTGGTCACGGATGCGTGTGGGGTTGCTGCTCAGTCTGTAACG
GCAG

In some embodiments, the intronic domain that promotes trans-splicing primarily in the brain comprises or consists of sequences from the gene OCIAD1. In some embodiments, the sequences from gene OCIAD1 comprise or consist of:

(SEQ ID NO: 110)
TAATCAGCGGTTGTAGGCCGCTTAGGCTTTAATCCAGCGTTGTTT
TGTGTAAAGAACTTAAAATGGTATTGTCTTAAGCGCCGTGTCAGC
CCTGACAGTCTTCCTGGTGTTTCTAGGAAGGATGCCGTTATGGGA
AATAAGAGGGTTTCATTTTTTCTCATACGGTTGTAGGGTAAAGGT
AGTTTATTCAGCAGGCTCCATCCATCCATGAATTCAGCTGACCTT
TTTTATTTCTTTTCTCTAATTAAGGGGTTGGCATGTAGTAGGCCT
TCATGGCTTATTGACTGAATGAATTATGGAAGGGCTCAGTTGCTG
TCCTCATTGTGTATGCCATTTCCTGTTTCCATGCAAATTGTTTTT
AAAATGAGAATAATCATGATCCCACGTAAACGTTCTGTAAAATAC
GGTTACTTGATCTGTTGGTCTGAGTCCCAATATATGAGTTTTTAA
AAAAAGAACCAAATAAAAAATGTATATTCTTGGAATTTTATGCAT
TTTT

In some embodiments, the intronic domain that promotes trans-splicing primarily in the brain comprises or consists of sequences from the gene SPP1. In some embodiments, the sequences from gene SPP1 comprise or consist of:

(SEQ ID NO: 111)
ACGTATTTGCTGCGATATTACTTATCTTCTCAGTCAAATCCATTG
GTTTACAAGTATTGATTGACTGCCTGCTATGAATCTAGGCCAGTA
CCAAGCACAGTATAGTTTTTAATAAATATAAGTTTATAAAACCAA
CCCAGATATTTTAAATATAATAATATCTAGGCATGTATGATGAGT
TATCGCATGTAAGATAAGTTATATGAAGTTGTGTGACTTTTTTTC
CATTAGTCCACATACTGATCTAAAAGCAGAAAATTCCAGCTTTTG
CTTTGTTTAGTGGATTGCTAAGTTTAAAATTCACATTGGATATTA
GTCAGAACTGTTTGTATGACCATAATATTCACAATATTGTCTGAG
ATATTAGCTGAGAAGCCCATTGTGAAAAGAAAGTCTATGTGTGCT
GTTTGTATCTATTGTGATTGTCAGCTGATGTTAGATCACATTTTC
TAACCAAACATAAGACCAACCAAACTCTTTATTATAATTATTTGA
CCAG

In some embodiments, the intronic domain that promotes trans-splicing primarily in the brain comprises or consists of sequences from the gene SPP1. In some embodiments, the sequences from gene SPP1 comprise or consist of:

(SEQ ID NO: 112)
TATTCTGTGTTCGTTTGTGTGTGGAGGGGTGTGTGTGTCTGTTTT
TCAACTGATTTGAAAATACAGGTGTTGAATCCTAATAATAAACCA
GAAAAATTAACATCTCCAGAGAAGATAGAGGTCATACTATTTGAG
GCAAGAATTAGCGTCTTTTTAATAAACGAAAATATGGCAAAGATG
CATTTTAGAAGGCACGTGGAGCTATAACAATTTAAGAAATACGTG
AAGAGCTCAAGGCTCAGCCTTCTAGAATCCCAGAAACTTAAAGCT
AGTAAAAAATTGGGGAAGTCTCTAAGGATATATGCCTGAAAATAC
ACACTGGTTATCTGTGAGTGTTAGGATTACTGGGTGGTTTTTAGT
CTATTCATTTTGCTTACCTTTATTTTCTTCATATTAGTTTTTAAA
AATTATAAATGTAACTTATACATCCATTCTCTCTGAGCCTGTATT
ACATGTGTCATGAGAATAGATAGATAGATATGAAAAAGTGAAGAG
AAAA

In some embodiments, the intronic domain that promotes trans-splicing primarily in the brain comprises or consists of sequences from the gene NREP. In some embodiments, the sequences from gene NREP comprise or consist of:

(SEQ ID NO: 113)
GCAGGGAGATGATCTCCTCCTCCTTTTTCCAAGGCTGCACTTCTT
GGAAGTGAAGCCGGTTTGGGTTTTGTTTCCCTTCCCCCTCCTCCC
TCCTCTTCCTCCACCCCTTTCCCCTCCCCCTCCCTCCCCCAATCC
CCCCTCCCCCCCCAATCTGTCTTTCTAGCATGTTGCCCTTTTTCA
ACCACATTTGTGTTTCAGGTGTAGAGAGGAGAGAGAGTGAACAGG
GAGCGGGGCTTTTGTCTGTAAGTATATGCCATTCCTATCCCCGGC
TGGGAGCGCTGGTTTCTCTTTTGTGTCGATTCCTTGCCGTTGCTC
TTAGCTGGACCCTTCAACCCTGCAGAGTTCATGCTTCTTCCCTCT
AAATTTTAAAGAGCTGAAGCGTGTACCTTGGAGAAATTCTGGCAT
TTTGGTTGTGCTGCATTCAGGCTGGAATTTAACTTGGGTTTTTTC
TAAAATGCACAAGTTCGGAGGGAGAAATGAATTCTTTTGGGAAGG
GGTG

In some embodiments, the intronic domain that promotes trans-splicing primarily in the brain comprises or consists of sequences from the gene NREP. In some embodiments, the sequences from gene NREP comprise or consist of:

(SEQ ID NO: 114)
ACGGCCCTGGCGGGCCGCCGGGCTGTGCAGCACCCTGGCCGCCTT
CACGCTTGGCGCGCCCCCAAACCGCCTCCTCCACCTCCTTATTTG
TCTGAAAAGCAACCGCACGTCGAGGCGGAGAGGCTGGCCGGGGGC
GGGGAGGAAGGCCGGGGATTTGCGCGCGCCGTGCTCCCGGGGCGG
GGCCGGGGCGCAGAGCTGCGGGTCCGCTTTGTCTCCCCGGGGCGC
CGCGCGGTGCAGTGGGGACGGCGCGGCGAGCGGGGCGCCCGGGGC
GCGGGTGCGGGGTGTCGGGGCGCGCGCGCCTGTTGGCCGCTCGCC
GACGGGGCCCAGAGCCGCCGATTCCTGCCGAGTAAGCAGTCTGGC
TCCTAGGCGGCTGCACAGTGGCAGCAAGGCTGGGGCGCTGGCTCC
GCGCCAGCTCCATCAGAATTAGCTCATTGTTCACTAGCACCCTAG
CGGGAGAGAGAGGGAAAGGGAGAGAGAGAGACTCTTAGAGAAAGA
GAGA

In some embodiments, the intronic domain that promotes trans-splicing primarily in the brain comprises or consists of sequences from the gene HSD17B4. In some embodiments, the sequences from gene HSD17B4 comprise or consist of:

(SEQ ID NO: 115)
CAAATCTTTACCATCCCTAAGGGGGACTTTCCTTCCCAGGGGCTA
TCTCTGGAAGCCCCTCAAGGATAGGGGCCGCATGCTGTTTCTCTA
GGTCAGCAACTAAACCCAGAAAACGTTTATTGAGTGAATGATGAA
ACGACAGGTGAATAGATGAACGCAAGGTGTCGAGTTAACTATTCT
TCTACACAAGTCCTAGCAGCTCCCATTGCTTCCAGCCGCAGAAAT
GGCCCCTGGAAGGCAAGTCTTCCAGCGAGTGGAGTCACTCTTAAC
TACATTTCCCAGGATTCCAAGGGAGCCGCGCGCTCTGCGCTCATC
TTCCTACCAGAAATCGGCAAGTCACTGACCCTCGTCCCGCCCCCG
CCATTCCCCGCCTCCTCCTGTCCCGCAGTCGGCGTCCAGCGGCTC
TGCTTGTTCGTGTGTGTGTCGTTGCAGGCCTTATTCATGGGCTCA
CCGCTGAGGTTCGACGGGCGGGTGGTACTGGTCACCGGCGCGGGG
GCAG

In some embodiments, the intronic domain that promotes trans-splicing primarily in the brain comprises or consists of sequences from the gene HSD17B4. In some embodiments, the sequences from gene HSD17B4 comprise or consist of:

(SEQ ID NO: 116)
TACAGCCCGCTTCTCCCCAGCACCCCGGTGTGGGCTTCCCAAGGTCCTG
CCTGAGAGGAGAGGCCAGGCTGGGCTGCTGATTGCAAAACTGGGTGAAA
GTTCTCCCTGACCCTTATCTGTGGGCATCGATTGTTACTCTTCCTGCAA
TTAACTCTCTTAGATCTTTGCCTAGTCTTTTAAAGGACTGAAAAGCCGC
GAGGGGCGGGGGCTGGAATTCGCCCCCTGAAGCGCAGAGATGTCAGCTC
CTGAAAAGTCATTCGGTCGTTCAGTGTTTGTTTCCCTCTGTCGTAAGAT
TTTAAGTTCGTGAGAGGACCTTCTTTAAAGAGGGCGTCTGATAAGAGCC
CTTCCCCGTTGGAGTTTGTATGCTTAGCAAGTCACAATCTGTTCTCGAA
ATCCACTGGAGTCTTGGCAGAGGTTGTAAGCTCAAATGCGCACAGGGGT
CAGGCGTATGATGGAGAAAGAAAATGGGAGTAGGATGGGCACATCTGAG
GAACTGGAG

In some embodiments, the intronic domain that promotes trans-splicing primarily in the brain comprises or consists of sequences from the gene LMNB1. In some embodiments, the sequences from gene LMNB1 comprise or consist of:

(SEQ ID NO: 117)
TGGCCGGGGACACTCTGAGTTTCGCGTGTGGCTTTTAGGGACGTTTATA
TTTGAATTTCCCTGAACCGCCGAGTGTGGGCGGTGGCGCAGATCCGTCC
CGGAAACCTCCGGGCTCCTTCCCGCCTTTCTCAGGCCCGGCCCCTCCAA
GGGGTCCCCGCGGGGCGGCGGGAGGGCCCTGGGCCCAGAGCCGCGCGGG
TGGGCAGTCCCAGGCGTCCTTCCTTACAGCCCTGAGCCTGGTCCGGGAA
CCGCCCAGCCGGGAGGGCCGAGCTGACGGTTGCCCAAGGGCCAGATTTT
AAATTTACAGGCCCGGCCCCCGAACCGCCGAAGCGCGCTGCCTGCTCCC
CATTGGCCCATGGTAGTCACGTGGAGGCGCCGGGGCGTGCCGGCCATGT
TGGGGAGTGCGGCGCCGCGGCCCGCGCCACCTCCGCCCCCCGCGGCTTG
CCTCCAGCCCGCCCCTCCCGGCCCTCCTCCCCCCGCCCGCCGCTCCGTG
CAGCCTGAG

In some embodiments, the intronic domain that promotes trans-splicing primarily in the brain comprises or consists of sequences from the gene LMNB1. In some embodiments, the sequences from gene LMNB1 comprise or consist of:

(SEQ ID NO: 118)
TGAGTGGGTGTGTGTGTTTTCTTACAAAGGGTATTTCGCGATCGATCGA
TTGATTCGTAGTTCCCCCCCGCGCGCCTTTGCCCTTTGTGCTGTAATCG
AGCTCCCGCCATCCCAGGTGCTTCTCCGTTCCTCTAAACGCCAGCGTCT
GGACGTGAGCGCAGGTCGCCGGTTTGTGCCTTCGGTCCCCGCTTCGCCC
CCTGCCGTCCCCTCCTTATCACGGTCCCGCTCGCGGCCTCGCCGCCCCG
CTGTCTCCGCCGCCCGCCATGGCGACTGCGACCCCCGTGCCGCCGCGGA
TGGGCAGCCGCGCTGGCGGCCCCACCACGCCGCTGAGCCCCACGCGCCT
GTCGCGGCTCCAGGAGAAGGAGGAGCTGCGCGAGCTCAATGACCGGCTG
GCGGTGTACATCGACAAGGTGCGCAGCCTGGAGACGGAGAACAGCGCGC
TGCAGCTGCAGGTGACGGAGCGCGAGGAGGTGCGCGGCCGTGAGCTCAC
CGGCCTCAA

In some embodiments, the intronic domain that promotes trans-splicing primarily in the brain comprises or consists of sequences from the gene SKP1. In some embodiments, the sequences from gene SKP1 comprise or consist of:

(SEQ ID NO: 119)
GTACGCAAAGAGAACCAGTGGTGTGAAGAGAAGTGAAATGTTGTGCCTG
ACACTGTAACACTGTAAGGATTGTTCCAAATACTAGTTGCACTGCTCTG
TTTATAATTGTTAATATTAGACAAACAGTAGACAAATGCAGCAGCAAGT
CAATTGTATTAGCAGAATATTGTCCTCATTGCATGTGTAGTTTGAGCAC
AGATCCCAAACCTTACGGCCAAGTTTCTTCTAGTATGATGGAAAGTTTC
TTTTTTCTTTGCTCTGAATAAAACTGAACTGTGGGTTCTCTATAAGTGG
CATTTTGGGCTTTCCCTCTTTTTTGTAAAGCAATGTCTGCCTAGTTTAT
TGTCCAGTTAACTTTAGTGACCTTTTAAAAGTTGGCATTGTAAATAAAA
CAACTTGCAAAAAAGTTTTCTGGAATAGAATTAACAAAATATTATCTTT
ATTCATGAGTTGGAAACTGGAAAAAGGCTTCTTGAAGTAAATGTTCTGA
GTGGAGCTA

In some embodiments, the intronic domain that promotes trans-splicing primarily in the brain comprises or consists of sequences from the gene SKP1. In some embodiments, the sequences from gene SKP1 comprise or consist of:

(SEQ ID NO: 120)
TATAGTGCTATAACTGCTTACGTTTGAACTCTTTAAGTGTGGACAAATA
TTTTTGCCAAGACCTGATGTGGTATTTGTGTGCATGTGTACACACATAC
ACAACAGTAGTGGCCGTTATTTCACATTTGAGCTAGTATAGTTTGGTTA
AGGAAGCAGAAGGATTCAACATTATAGAGGTATAACAAAGAAGGTCACA
TAAAAGTCTTTCACAGAAGAACTTAGGAGTGATTTATTCTTCTCCCTTT
AATTTCTTAAAATTAGCATTTTAAGCTTTATTAGATACGGAGTGTCAAT
CATTAACTTTGGATTTAGTTTAAAACATCAAGTATACTTTAAGGAAAAC
AACTGTCTCTTTACAGGCTGCAAACTACTTAGACATCAAAGGTTTGCTT
GATGTTACATGCAAGACTGTTGCCAATATGATCAAGGGGAAAACTCCTG
AGGAGATTCGCAAGACCTTCAATATCAAAAATGACTTTACTGAAGAGGA
GGAAGCCCA

In some embodiments, the intronic domain that promotes trans-splicing primarily in the brain comprises or consists of sequences from the gene REEP2. In some embodiments, the sequences from gene REEP2 comprise or consist of:

(SEQ ID NO: 121)
CCTTTCAGATTAGAAAGGAGCAGTCCCATGAGCCCCATGGGGACCCCAG
TGCACTAAGAGTGAGGGGCTGTGCAGGGTCACACATGGGGCCGGTGCTG
CTGGACACAGGGCACCTCCCAACTCCCTGCCCTGTACTCTGCGCTTGCC
CCTGTCCCAACAGGTTCCCCTTCTACTTTGAACTGAAGATCGCCTTCGT
GATATGGCTGCTGTCCCCTTACACCAAGGGCTCCAGCGTGCTCTACCGC
AAGTTCGTGCACCCAACGCTGTCCAACAAGGAGAAGGTTTGCCCCCACT
CTCAGCTCACCTCCCAGCCTGCCCCCAGCCAAGGCAGTCCAGAGCCTGC
AGCCTCATACAGACTGGCCCTCCCTGTGGGGCCCAGAGCCCAAAGTGAC
TTGGCAGGGCTGCCGTGGCCTCCCGGAGCAGGCAGGGGCCTCTGTCCAG
GGGTGAACAAGGGTAGGGCAGGACCTCTCCTCTTCTCCCTTCCCCTCCA
ATCCCATAG

In some embodiments, the intronic domain that promotes trans-splicing primarily in the brain comprises or consists of sequences from the gene REEP2. In some embodiments, the sequences from gene REEP2 comprise or consist of:

(SEQ ID NO: 122)
TGAGATGGGGGCAGGCTCAGACTCCCAGGGCCCCTACCTTGTACAAAGG
GCCCTGGCCAGGCCCCTTTGCATCCACCCTGTACTCGGGGCTGTAGAGA
CTCTGGGCTCATGCAGCTGGAGGCTCCAGTCCAGGCTCTACTGCTATCT
GGCCGAGTCACTTTGATTGTCCGGCCATTTCCCCATCTGTCCCCAGAGG
CAAAGTGTGGCCCTGCCCCAGCCCTCAGCTCTGTCTGTGTGGGACTGGG
TGAGGACAGTGTGGGGAGGGCACCGAGCCTGGGGCTGCTGCCAGCACCA
TGGTGACCTCTATCTCCACCCCGCCCCTTCCCCCCGGCTCTCCCGGTGC
GTGGTGGTGACCCTAGGGCCAGGGGGTGCTGTCAGAGAAGCTCCGCAGC
TTCAGCATGCAGGACCTGACCCTGATCCGGGACGAGGACGCACTGCCCC
TGCAGAGGCCTGACGGCCGCCTCCGACCCAGCCCTGGCAGCCTCCTGGA
CACCATCGA

In some embodiments, the intronic domain that promotes trans-splicing primarily in the brain comprises or consists of sequences from the gene MATR3. In some embodiments, the sequences from gene MATR3 comprise or consist of:

(SEQ ID NO: 123)
CCCTTTGTGGCCCCAATATCTGGCGCTTTCCACCATTCCTTGGCAAACA
GAACAACCAGAGAGCATAAAAGTCGAGTCTAAGCCAGGGACTCTCGCGA
ATCCCTTCTAAGGCATACCCTATTTAAACTTTGTATTATGCTATTCAGG
AGGTTGACAAACAGCCTTTTCGGGAATGGGACTCGAACTCGTAAGCCAC
GTCTGTTCCTCCATTTCCCGCCTCAGCTGGATGACGGAGAGGGATCTCC
GGGATTAAAGAACAACGACCGCAGGCGAGTGCACGAGCGAGGTGCGCGC
GCTGAGTAGGGGGAGGGGCCTGGCGCGCGCCGAACCAGATCGAGGGGGT
GGGGGCGAGCTCTCTCGCGAGAGTGGGTTTGTTCTTGGGCTGCAGCCGC
TGCCGCCGCTTCTCGCCAGCGCCGTTGCTGCGGGGGATTGTGGGAGTCT
CCGCGTCCCGCTCGCTGGGAGAGAGGTACCTCTCCTTTTCCCTCTCCCT
TTCCCTAAG

In some embodiments, the intronic domain that promotes trans-splicing primarily in the brain comprises or consists of sequences from the gene MATR3. In some embodiments, the sequences from gene MATR3 comprise or consist of:

(SEQ ID NO: 124)
TGAGCGGTCCGGGAGGGAAACACGCGGCCGGCCAAGGGCCCAGGGTGCG
GCGGGAGATTTTGGGTGAAGAGGTGCGCTGACCGGCCGAGCTTCCTGCG
CGTCCTGGGCTCGTTGTGGGCGGGGGCGGGACGACTAGCCCGTTACACG
CGGGCCGCGGCGCTGAGGGGGAGGGAGGAGCTCGCTCGAGGCCTGAGGG
ACAGACTGTACACTGGGGGCTTGCCGTTTGAGGAGGATGGTTAAGATAT
CTAAGTCCTTGGGTAGCGCCAGTGCCAAATAACCCGTTCGCCTTTTTTT
CTCTGAAAATGTTCCCCCAAAATGGAGGATTTCGCAGACTCTGAAGAGC
CTGCTTGTTTGAGCAGCGTTTCTTTCTTTTCCCGGTGTCTCGGCCTTCA
CGCCGCTCCTTGCCCATCGCCTCAGAGCGCCCCCTAAGACTCCCGGCCC
ACACGCGCGCGCGCGCGCGGCGTTAGCTGGACATCAGAAACAATTCCTC
CCCGCCCCC

In some embodiments, the intronic domain that promotes trans-splicing primarily in the brain comprises or consists of sequences from the gene CAMK2A. In some embodiments, the sequences from gene CAMK2A comprise or consist of:

(SEQ ID NO: 125)
GTAACAGGTCCTCACCCTCCACAGTCCTGGCCATGGGACTAGCTCTGCC
TGGGTCTCCACAGAGGCCAGGAGACTCGCTCACTAATTCTTTCCTTCAT
TGATTCAATAAGCATTTATTGAACACCTACTGCATACTGGACAGCAGGT
TGGGCTTGGGGAGTGAGCGGTGAATGACTGAGCCCTTGCTCTAGTGGAG
AGGACAGACAAGTTCCTGGGGCGTTATGGGGCAGTGGTGAGTGCCATGC
CTGGGAAAGCCTCAAGATGGTGGCAGAATGCAGCAGGACAATGGTATTC
TGGGTCTGGAGCCAGGGGCGTTTAAGCTGAGACCTTGGAGGATAAGCAG
GTATTAGCCAAGTTAGAGTCAGAATGGGAAAGTGTGGCCGGGCGCAGTG
ACTCATGCCTGTAATCCCAACACTTTGGGAGGCTGAGGTGGGCAGATCA
CCTGAGGTCAGAAGTTCAAGACCAGCCTGGCCAATGAGGTGAAACCCTG
TCTCTACCA

In some embodiments, the intronic domain that promotes trans-splicing primarily in the brain comprises or consists of sequences from the gene CAMK2A. In some embodiments, the sequences from gene CAMK2A comprise or consist of:

(SEQ ID NO: 126)
TCCCATCGCCGGAATGGGACACTGTCACCCCGGAAGCCAAGGATCTGAT
CAATAAGATGCTGACCATTAACCCATCCAAACGCATCACAGCTGCCGAA
GCCCTTAAGCACCCCTGGATCTCGGTGAGCCTTCCTCAGCAGGACTTCT
CCCAGGAGCCCCTCCAGACCTCATGATCCAGGCCCCCACAGAGTTCTGG
CCCTTCCCCTGGCCCATGGGGCCAAGAGGGCTTAATCCAAGAGACTGCC
CCTGCTGTGGTTCTCAGGGGTAATGTGGAAACGAGGAGTGAGTGTTCTG
GGTTGGCATACAACTGATGGTGCTACTGGCATTGAATGTCTGGTGCCAG
GATGCTAAACACCCTGAAGCATCTCCTAGAGCAATGAATCGTTCTTCCC
AAGATGTCAACCACACCTCCATTAAGATGCCCTGCTGAAGGGTACCCTT
GGGTGGGCCTGTCTTCCAGCCTGCCTTCCACTCACAGCCCTCTCCCCTA
CTTCCTCCA

In some embodiments, the intronic domain that promotes trans-splicing primarily in the brain comprises or consists of sequences from the gene CYFIP2. In some embodiments, the sequences from gene CYFIP2 comprise or consist of:

(SEQ ID NO: 127)
AAACCAGTTTTCAGAGAATTAGAGTTGGATATCAACTGGACAGCACCCA
CAACAGATTAGACTTGGATTCTTCCTTTGAGGACTTGAGAGGCCGTCAT
GAGAAGAGTGCTTCTCGGCTCCTAGGGATGGTGAGACAGGCCCTCCAAC
AGGGCTGCAGATGGGGGACTGTCTGAGGCACTGACGGCCACTGTGGCTG
CTTTCCCACAAAGGAAGCCCTGTGGCAGGGATGGTGGGTCACACGTTTC
TGTGGCTGCTCTACCCGCATCAGTGCCAGGCATGTGATGGGGGAGATCC
CAGGGAAGCTCCAGAGGCAGCGAAGACGAACGCGGTTGCTGGGAGGTTG
GAGTGTCTCAGAGGTGGCCCCCGGCAGTCGCGGCGGCTTTCCCACCGTA
TACAATACCTCTTGCTTTTCTCTCTCTCTCTCTCTTTCTCTCTCTCCCT
CTTCCCTGTCTCTCCTGCCCTCCCATTCCCTCTTCCTTCTCTCCTATCT
ATGTCTTAG

In some embodiments, the intronic domain that promotes trans-splicing primarily in the brain comprises or consists of sequences from the gene CYFIP2. In some embodiments, the sequences from gene CYFIP2 comprise or consist of:

(SEQ ID NO: 128)
TATCACACCGCCTGGCTGGGTTGGTTTGGGTACCCTTCTCGAGGAGGCA
GCAGGAATGGACTAGCCTGGCTCCTCAAGGCCCCTTCCAGCCCTAAGAT
TCACAGACAAATGATGTGGAGAGATCTAGATTAGCAAAGAGAACCACCC
CACCGCACTTGCATCCTTTGCACTTAAGGCACCCTGTAAGCGGGAGTTA
ATTGTCTAGTAGCCTCCTGTGTAAGTACCAATGTTAACCCGCCTTCCAC
CCTGAACTTCAGCAGGCTGTGCAGTGCAGCTGATGCCACCAAGGGGGCG
CCATCGCTCAGCCATGGGGTCAAAACGCTGAGGGTCAGGAACAGGTGTG
GGAGGGGGTTCGTTCTGATCTTATCCCATTTCTCAGTTCCTCAGAATGT
TAGTGCTGAAAGGGACTGGTGTGAGAAGGCATCTGGTCCAACCCCCTCA
TGTTACATGAGACAACTGAGGCCCAGAGTGGGGGCTTTGTTGCCTAAGG
TCACACAGC

In some embodiments, the intronic domain that promotes trans-splicing primarily in the brain comprises or consists of sequences from the gene PTTG1. In some embodiments, the sequences from gene PTTG1 comprise or consist of:

(SEQ ID NO: 129)
GCCAATTGGGCCGCGAGTTGTGGTTTAAACCAGGAGTGCGCCGCGTCCG
TTCACCGCGGCCTCAGATGAATGCGGCTGTTAAGACCTGCGTGAGTGAA
TGGGAGGGTCGCGGGTGGTTAGTTGAGCCGGCTCCGGCGGGGAAGGAGG
CGGGCTGCGGCTGCGGCTGGGGCTGAAGCTGGGGCTGGGGTTGGGGGAC
TGCCCGGGGCTTAGATGGCTCCGAGCCCGTTTGAGCGTGGTCTCGGACT
GCTAACTGGACCAACGGCAACTGTCTGATGAGTGCCAGCCCCAAACCGC
GCGCTGCTCGGGACCTTAGAGCCTCTGACTCAGGCTGGAAGATTTGAGA
GCTGGATTAAGTACTTGTTGGCTCACGCCCGTGACTGTTCCGCTGTTTA
GCTCTTGTTTTTTGTGTGGACACTCCTAGGATAGAAAGTTTGGTATGTT
GCTATACCTTTGCTTCTCCCACCTTCCCCAATATCTAATATGTATTTCT
CATTCTTAG

In some embodiments, the intronic domain that promotes trans-splicing primarily in the brain comprises or consists of sequences from the gene PTTG1. In some embodiments, the sequences from gene PTTG1 comprise or consist of:

(SEQ ID NO: 130)
TAAGTATACAAGGCTGCAGTCGGATACACTGGTATTGTGGACGTGGCCT
GGAGCTGGACGAGACATTTAGTGTACTTTTTGGGCAATTGGAGTCGTTT
GTTATTGGTCCTTTTTCATTTTTAATATCTTAATGAGATGATTTAAGGA
AGTTACTGAATCTCTGCTATTAGGCCTATCATAGCCATGCCACTACCAA
AAGTGGGGGGTGGGTTTGGGGGTGAGAGGTCAAGTTTATATACACAGTA
TTTAAGTTGTACAGGTATTTTGCTGCTGGAATATTCTTATGGTAAGACT
TTTTCTTCAGCAATCAAAGCCTTAGATGGGAGATCTCAAGTTTCAACAC
CACGTTTTGGCAAAACGTTCGATGCCCCACCAGCCTTACCTAAAGCTAC
TAGAAAGGCTTTGGGAACTGTCAACAGAGCTACAGAAAAGTCTGTAAAG
ACCAAGGGACCCCTCAAACAAAAACAGCCAAGCTTTTCTGCCAAAAAGG
TAAGTGTTG

In some embodiments, the intronic domain that promotes trans-splicing primarily in the brain comprises or consists of sequences from the gene SQSTM1. In some embodiments, the sequences from gene SQSTM1 comprise or consist of:

(SEQ ID NO: 131)
GCCGCGTACCAGGACAGCGAGAGGAAGGCGCACAGGCAGAAGAGC
AGCAGCGTCAGGAAGGTGCCATTGCGGAGCCTCATCTCCTCGGGT
GCGCGGCGGGCGCCCGCGGGGCCGAGGCTGCATGGCCCGGGGGAC
CGGGGCCGGGGCGCAGGGGTCGGAAGGCGGCGGCGGCGGCGGCAG
GGGCCCCGGCCCCGGGTCGGGGAGGGGCGGGGGGCCCGGGGCCGG
GCGGGGACCGGGCCAGGGAGCGCGCCGGCCGCCCCTCAGGGCGCA
AGCTTTGTGCCCTGTACTCAGGGAAGAGGAACAGGCTCAGAAGGG
CAGAGGCAGGTATCAGGCTCACTGCAGATATCAGGGGCGCGGGAC
ACTGGCGGCCTCGCCTCCGCGGCAGGGCCGGGCCGGGCCGGGCTG
GGCTGGGCTGGGCGGCGAGAGCCGCGGCCCGGCCTGGATCTGGGG
CCTGGATCTGGGGCCGCCGCGGAGTCGACGGCGCAGGGCGGGGCG
GCCC

In some embodiments, the intronic domain that promotes trans-splicing primarily in the brain comprises or consists of sequences from the gene SQSTM1. In some embodiments, the sequences from gene SQSTM1 comprise or consist of:

(SEQ ID NO: 132)
TAAGGCTGGAGTGGGAGTGCAGGTCGACCGCAGCCGGGGCGGGGG
GCGGGCGGCGGGGGCGGCGCTCCGGGACCCCGGTACCCTTCTCAG
CAACATCTCCTCCGCGCGCGGACCCCGACCCCATTCGCACGTTCT
CGGGGCTCTTTCCCGGGCGTGAGGGGCTCTGGGTGGCGCGGGGGA
CTGGGCGGTTGAAGCCGGGAGCGACCGCTGCCTTCGCTGCCGCCC
AGGGCGCTTCCCCGCTCAGGAGCTTCCTCTGGGCCTCTGGACGGA
GGCGCGCAGGGGCCCGGGAGGCGGGAACGATGGGCCTTTCTAGGC
GGTATCAGGGCCGATGCTCGTGGATGCAGAGCAGTGACCAGCCCA
GAACCGTACCGGCTTCCCGGGGCAGGGGCGGCCCGAGAGCGCCGC
TAACGGGAGCAACGGGCCCTGCCCCCGGTGGTGTAGGGGCCACCT
CGCCCCGCCCAGCCCAGCCCGATATTGATGGGGGCCCACGCCTTC
ATTG

In some embodiments, the intronic domain that promotes trans-splicing primarily in the brain comprises or consists of sequences from the gene TUBB2B. In some embodiments, the sequences from gene TUBB2B comprise or consist of:

(SEQ ID NO: 133)
GTGAGTGCCTGGTGTGGCTGAGAGCATGAGGGATTCATTTTACGC
TGGGCAGTGGAGGCTGAAGAGGTGTGATTGCCAGAGGGAAAGCAT
GAAGAACATCCGCGGTGTGCCAACTTAGCTTTAATATAGTGTAAA
ATTGTTTGCCTTTCAGCTGCTAAGAGCTTGGTGTCCTGGCCTTCC
TTATTTATAATTATATTCATGAACAAATATTTTAACGTTTGGCCA
TTTTGGTGATAATCTGAAATAGTCTTATCTGAGCATTGACTCACT
GATCTATATTGAGGGGTTTGAGGTAAGGTATTTAGTACAATCACC
GGTGACTTAAGATTTCTCTTTCCCTCTGGCAGGCCAGAGTGGAGC
CGGGAATAACTGGGCCAAGGGCCACTACACAGAGGGAGCCGAGCT
GGTCGACTCGGTCCTGGATGTGGTGAGGAAGGAGTCAGAGAGCTG
TGACTGTCTCCAGGGCTTCCAGCTGACCCACTCTCTGGGGGGCGG
CACG

In some embodiments, the intronic domain that promotes trans-splicing primarily in the brain comprises or consists of sequences from the gene TUBB2B. In some embodiments, the sequences from gene TUBB2B comprise or consist of:

(SEQ ID NO: 134)
GAAATGTTGGGTTGTCGGAACTGAGTCGGGGACATTTCATTGTGA
GCCTTGGCGTTTCTGGGGGCATCGTTCATGGCTAAATGCAGCCTT
TCTGTCTCAGTTTTGGGAGGTCATCAGTGATGAGCATGGGATTGA
CCCCACTGGCAGTTACCATGGAGACAGTGATTTGCAGCTGGAGAG
AATCAATGTTTACTACAATGAAGCCACTGGTAATCACCCTTGCCC
CACCCCCACTCCCTTCAGTTTTTCCACCTTCTCCCCTTTCCCTTG
CGTGGGACCCCAGGGGTGTGGCCCTGGGAGAGGGTGGACTATTCA
GTTTAGGATCCCCGCTCTTTCCCTGCAGTATTTCATTGGGCTTCC
TTATGCCAACAGAGCCCTTTGAGAACCTGATGGTGGGTCTCCCTT
TGTTTGGGGCAACATCTGCATGATGGCAGCAGGCGCCAAGCCCTT
CTCTGCAGAGGTCCTTGCCTGAGGGTCTAAGTCACTGTTGTTCCT
TGCA

In some embodiments, the intronic domain that promotes trans-splicing primarily in the brain comprises or consists of sequences from the gene GABBR1. In some embodiments, the sequences from gene GABBR1 comprise or consist of:

(SEQ ID NO: 135)
GTGTGTGGGATGTGGGCAAAGGAGGGCAGGGATGCACAAAGGCAG
GAGGGAAGGCAGGGGTAGAGGGCTTGGAGGGAGAGGGGTCTTTGG
AAGAGGAGGTAGAGAGCTTGTCAACCCAGTTTGAACACCCTACTC
TTTGTTATTGCACTAATCTTTTCTGAGAATAGGGGAGAGTTGCTC
TTTTGCTATGAGGAGCTTAGGGCCCAAAGCACAGAAAGCACAGAT
GAAGAACTTGTGTTCAGCAGAGGAACAAGTGGGGGTAACCCCACC
TCCAGACTTGACATTATCTTTTAGATCCCCCTTGGCCTTATTAGC
ATTGTTCGATTCATGGTCACAAATTGCAAACCTACCCTCTGCCTG
GAAAGCCACCTTCCCACCTGTAGGGTAAGGGTGAGGCATGTGTGG
CCCAGACTGGCCTATTTCTAGATATTCAACAAGCCCTTGCCTGAC
TGACAGCAGCTTGCCACCATTGCTTTCCTGTGTGAATCCCAGGAA
AAAG

In some embodiments, the intronic domain that promotes trans-splicing primarily in the brain comprises or consists of sequences from the gene GABBR1. In some embodiments, the sequences from gene GABBR1 comprise or consist of:

(SEQ ID NO: 136)
GTATGCCACAGTGGGCCTGCTGGTGGGCATGGATGTCCTCACTCT
CGCCATCTGGCAGATCGTGGACCCTCTGCACCGGACCATTGAGGT
ACCACTGGAGAGGAGGTGCTATGGTCAGGAGAATGAGCAGGGCTC
AGTGGCCATCAGGGCCCTGGGGCTGTGTGTGTCTTGAGGGATGAA
GCTACTTGGAGAGAGTGCCTTCCTCGTATTGGAAGCTCTTCCTTT
CCTTCCTAGAAGGAGCCCCTCATAGGCCTCCAGATTCAGCTGAAG
AAAGGAAGGGGTGGGAATCTGGGAAGGGTGTGTAGAACTTCCAGG
CATCAGGGAAAGTGGGGAACAAGCACCTCCAAGGGTTCAGGAAAA
CATTCTTAGGCCTAGAATGAGATTTGGCATCAGCATTGAGGGTCT
CATAGGAAAACAGTTGGAAGCCAGAGACTGAGAAGCGTTGAGGAG
AGGAGGGGAGGCTGGCAACCATCTTTCTTGTGACCTTGTTTCTGC
CCTA

In some embodiments, the intronic domain that promotes trans-splicing primarily in the brain comprises or consists of sequences from the gene RPP21. In some embodiments, the sequences from gene RPP21 comprise or consist of:

(SEQ ID NO: 137)
TTGAGGAAGACAAAAGCACTGAAAATTCATAAAGGGACCCTGGGC
ATGGATTGCTGGGGTTGTGTTTAGAAACCGATGAGTTTGTGAGGC
CTCCGGGAGGCTCCCGAGGGCGCGGGGACTACGTTTCCCAGGAGG
CCTCGCGCGGACGCCCGGGCGGGGCTGTGCGAGGGGTGGGGCTGC
GGGAGGCCCTGGAGCGCGGCGGTGATGGCGGGGCCGGTGAAGGAC
CGCGAGGCCTTCCAGAGGCTCAACTTCCTGTACCAGGTGAGTCTG
CGACAAGGGCCCCACGGGGACGGTGCTCGGCGTCCCAGAGTGACT
GCTCCCCTCCCGCAGGCCGCCCATTGTGTCCTTGCCCAGGACCCC
GAGAACCAGGCGCTGGCGAGGTTTTACTGCTACACTGAGAGGACC
ATTGCGAAGCGGCTCGTCTTGCGGCGGTGAGACAGCCACGGGGCG
GGCGGCGGGCGGGACGCGGGAGGAACGCGAGAGGGAGCGCGGGCG
CCAG

In some embodiments, the intronic domain that promotes trans-splicing primarily in the brain comprises or consists of sequences from the gene RPP21. In some embodiments, the sequences from gene RPP21 comprise or consist of:

(SEQ ID NO: 138)
GATCCCTCGGTGAAGAGGACTCTCTGTCGAGGCTGCTCTTCCCTC
CTCGTCCCGGGCCTCACCTGCACCCAGCGCCAGAGACGTGAGTGC
TCCAACGGAGGTGGAAGACTGCGGAGCATTGGGGGCGCGGAGGGG
GGCGGGGTGGGGGGCGGGCACTGGAGGCCAACAGCGCCTTTCTCA
CTGTAGATGGATGTTGGGTGTGGGATTCGCAGGAGTCTTCCTTCT
TCGGGTTTGGATTAAGTTCCTAACGCCACTTGCACAAACTAGGGT
TTGGGCTCGGCTGTTTTTTTTTTTTTTCTTCCAGTGTGGGCAATA
AATAATAACTTTTAAGAGGCAACCCCACCCATGCACAATAATAGA
TGTTGTTCGGCTTTGTGGAGGACGATTCCCATCACCATTCATTTA
TTAAGCAAATACTTATTTTCTAAAATGTGTCAGGTACTGTGCTAG
ATTCATTATTCTCATTGAAATTACGGTCTGATGGGACAGACTAAG
AAAC

In some embodiments, the intronic domain that promotes trans-splicing primarily in the brain comprises or consists of sequences from the gene TCF19. In some embodiments, the sequences from gene TCF19 comprise or consist of:

(SEQ ID NO: 139)
GGAAAACTTGTGAACTAATCAGAAAAAGTGGAAGGCGGGAGATCT
TGGGGCGCTGTCCAATGGCGCGGAAGAGAACACATGAGCTGGCCA
ATCGGGAACGGCACGGGGGCGGGCTCGCTCGGCGCGAAGTTCGGG
CCCGGGAATTCCGAAGGAGGGGTAGGCGCTGCCCGCGCGCAGAGG
CCGCGCCCCTCCTGGCCCCGGCTTCTTGGCTGTCAAACAGATGCA
GCAACGTCGGCTCCTGCCGAGGAGCCCAAGGGGTCCCGGGATCCG
CCGCACAGGCTGGCACTGCTTGAAGAGGAGGCTACTCGGAGACTG
CGCCGCGCGGGTAGATCCGAAACGGGGCTGGGGCGGAGTGGGAAA
AGGCCGGGTATGCCTTGCATGATCGCGGGGAGCTCCTTCCTGTTT
TTATCCCACCTAGAGAAGCCGGGAAGTAGGGGTTTAGGTCCAATT
TGTTGGAGTACTTAAGGACTCGTTTGCACTTTCTTTTGGGGGATG
ACAG

In some embodiments, the intronic domain that promotes trans-splicing primarily in the brain comprises or consists of sequences from the gene TCF19. In some embodiments, the sequences from gene TCF19 comprise or consist of:

(SEQ ID NO: 140)
TTGGAGGCTCCCCAAATATTTTGCGATCTGAGGATCCAGCTCAAG
TGAGGTGCCATAGGACGTGTTCCTGAGTTTGCATTGCACGGAGAC
CTTCCTGGAATTTTTCATTTGCAAGTCGGCTTAACCAATTTTGCA
TTGAGTCCTAGGCTGCTTGCACTCTGAATTTGGGCTATTCAGGTA
GTGTGCTCAAAGTTGAAACCGCATACAGCACAACTCAAGTTTGCA
TCAGACTGGGAAGCGAACTTAAGCCAGCGGTGCGTGGCCCAGGAG
TGGGAAAGGAAATGGATGCCTGAAGTGGAAGAGGTGGTGCAGAGG
GGGCACCGCCCATGCTGCCCTGCTTCCAACTGCTGCGCATAGGGG
GCGGCAGGGGCGGTGATCTCTACACCTTCCACCCCCCCGCCGGGG
CTGGCTGCACCTATCGCTTGGGCCACAGGGCCGACCTGTGTGATG
TGGCCCTGCGGCCCCAGCAGGAGCCTGGCCTCATCTCTGGGATCC
ACGC

In some embodiments, the intronic domain that promotes trans-splicing primarily in the brain comprises or consists of sequences from the gene ITPR3. In some embodiments, the sequences from gene ITPR3 comprise or consist of:

(SEQ ID NO: 141)
GCGCCGCTGCCCTGCTGGCCCACTCGCTGGCTGCACGTTCCTTCC
TCAGCAAGCGATTTCCTCAGGCACATGTGCTGTGCCAGGCACTGT
TTTGGGGCCTAGGGTCCAGTAGGGCACCAGACAGCAGGGCCGGGT
TCATGGGAATGGGACCTGGCCCGGTGCTCACAAGGGCCTTGCACT
GGGTTAGATGCTCTGCTGCTGAGCAAGGGTCCTGCTCTTTCATTC
TGCACTGGGCCCTACAAGTTATGTAGCCAATCCTTCCACACAAAC
AAGGTCCCTGCCCTCGTGGAGGTTTCGCTCTGGTGGGAAACAGCC
CACAAGCTAAATAACTATGTAATGTAAGCCACTCTTCTGCCCAGC
GATGCTTCCTTTCCTGGACCTCTGCCTTTCTAGGGTATTGGGTCC
TTACCTCGGGGTTTGGGGGCAGCGTTCCAGAGCAGAGCTGGGCCC
TTGGCCCACCTGTGACTCTCTGTGACCCCCAGCCTGTCTGCCCCC
CCAG

In some embodiments, the intronic domain that promotes trans-splicing primarily in the brain comprises or consists of sequences from the gene ITPR3. In some embodiments, the sequences from gene ITPR3 comprise or consist of:

(SEQ ID NO: 142)
TGGGCCCGAACCCCCTCCCCGGCCGGCGCCTGCTCCTCCCTCCTC
CCTTGCCTGGGTAGAAACTCTGCTGGTTGTGTGAACTATTCCTGC
ACCTGTTGGTAACTGCCACCCTGAATAGCACTGGGTGGCTCAGCA
CTGGGAGGCCCTAGCCTCCCTAGCCCTTCTCTGGGATCCCCTAAA
ATTTGGCCCACTCTGGCAACAATGGGAATGACTCCTTGTACCACT
AGAGGCCCCCAAGACCTTCTCCAGTGACAAAGTCAGAACCTGTAT
TGAACCAGTTGTTAAATATTTTGAATATCACCTCGGCCCTTGCGC
TTCCTCACCCTGTCCGTCTGAACCCCCATCTAGCCTTCCCGGACC
CCAGGGTGATGCCCTGGGGCTTTCCCTCCTTGGGGAGCTGGGAGT
CCATCCCACCCATCTCTAAGCCTTGGGAGGGAGCTAGTTCCAGGG
TGGCGGTGCTGCCAGCTGGGGAAGGGGAAGGGTGGGCTCTGTCCT
TAGA

In some embodiments, the intronic domain that promotes trans-splicing primarily in the brain comprises or consists of sequences from the gene HMGA1. In some embodiments, the sequences from gene HMGA1 comprise or consist of:

(SEQ ID NO: 143)
CCTGCAACGGCCGGGCGCAGACACTTGCAGAGTCACCCTCGGGCC
GGGTCTGGAGCCTGATGCCTCGGGGGTTTAGCCCTAGCCGCTAGG
CGGGTAGGCAAAGTGTCGGGTTGAAAGGGTCTCCGGGAACCTACT
TCTTCCTGCTCGCCAACTTGCTGTGTGACCTTAGGCAACTCAAGG
CTTCTCTGGGCTACTTTTTTACGGGGGACGGGGAAGAAAACAAGG
GAACCCCCAAGATAATTTCCTAGGGCCTTTCCTGCAGAGTCCTTA
AGTATTTGAGGAGCACAGTTTGCATGCACAGTGTATGGAGGAGAC
AGGCTTGAGGCCCAAGCGGGCTTATTACAAAGTGAGGGATTGGGG
TTAGGCCCTAAATGTAGTTCCTCCAAGACAGGCCTCTGATGTTCA
GAAACAGGTGGAAAAGACGAACGTTTCTTGGCCTCACTGGAAAGG
GCCTCGGGCCTTCATTTAGTTCGTGGGGGGTTGGCTGAGGGGATT
AAAG

In some embodiments, the intronic domain that promotes trans-splicing primarily in the brain comprises or consists of sequences from the gene HMGA1. In some embodiments, the sequences from gene HMGA1 comprise or consist of:

(SEQ ID NO: 144)
TCAGCACTCGTTTAGTTGATTAATTTTGATGTTTAGTTTGAAGGG
GGACCTGTGGTGTAATATAAGATTCTAATCACTGCCTGTAATTGC
AGAGCAGGCCAAGCCACTAATGACGGAGCAGGATAAATCACCAAC
CACCTTGTTTAAAAGGGTCTCCAAGTCTGAAACATTATAAGATAG
AATAATTACCAAAGTCCTGCTGTCTGGAGGGTCTCTTGATGCTTG
AAAATGGAGGTCCTGGGCCCTCTAGAAATCACAAGTTTGCAAAAA
TGTTTTTTTCCTCTCATTGGGGTAAAAACTCTTTTTCTTCTTTTT
TTTTTTTCTTTTAAATTAGACATGGGAGTCCCACCGTATTGTCCA
GGCTGGTCTCGAACTCCTGACCTCAAGCAGTCCTCCTGCTTTGGC
ATCCCAAAGTGCTGGGATTACAGGCATGAGTCACCTCACCTGGCC
CATAAATCATTTTCAAATGGTCTAGTTCTTATTTTGGTTAGAGTG
AAGA

In some embodiments, the intronic domain that promotes trans-splicing primarily in the brain comprises or consists of sequences from the gene KLC4. In some embodiments, the sequences from gene KLC4 comprise or consist of:

(SEQ ID NO: 145)
TTTGGCTTTCATTTTTCCTCTTTACAGGGTACCTCTCCTCCCTGT
TGGGCCTCCCAGATACACCCCAACAGGGTGCTTCTTTATGTGCCA
GAACTCTCACTTTTCCCATACCTAAGCATTTGCTCATCCCTTGGG
ATCCTTGTTCCTCTAATCTCTCTTCTGGGGAGTGCTCTCTCCCTC
TCTTCCCCCTGCCCCCTTGCTTCTTATAACTTGTGGCCTCCTCTT
GGGATGGCAGTGCCCTGCATTCTGGTATGAGTATGTCAGTGAAGC
CCCCTTACTGTCCACGCCAGGCTTCCTGTCTCCTCATGGAGGTCT
CCAATCCTTAAATGTACTCAGTCCTTCCAAAGGGAAGGAGGGAAG
GAGAAGGCTTGCGGGTTCAGGGTAACTCCTGTCACTTTTGTCTTC
TTCCAGTGACCAGAATAAGTATAAGGAAGCTGCCCACCTGCTGAA
TGATGCCCTTAGCATCCGGGAGAGCACCTTGGGACCTGACCATCC
TGCT

In some embodiments, the intronic domain that promotes trans-splicing primarily in the brain comprises or consists of sequences from the gene KLC4. In some embodiments, the sequences from gene KLC4 comprise or consist of:

(SEQ ID NO: 146)
AGTAATTTGTAAGGACAATATATACAATGTAATATAATAAGGTAA
AAATACAAAACAACAGTAGTTGTATTGAGTACAACTTTGTTAAAA
GAAATAGCAAAGTGGCCTGGGCACAGTGGCTCACGAGTGTAATCC
CAGCACTAATCCCAGCACTTTGGGAGGCTGAGGAGGGTGGATCAC
GAGGTCAGGAGATCGAGACCATCCTGGCTAACATGGTGAAACCCC
ATCTCTACTAAAAAAAATACAAAAAATGCCGGGCGTGGTGGCGGG
CGCCTGTAGTCCTAGCTACTCGGGAGGCTAAGGCAGGAGAATGGC
GTGAACCCGGGAGGCGGAGCTTGCAGTGAGCTGAGATCGTGCCAT
TGCACTCCAGCCAGGGTGACAGAGCGAGACTCCGTCTCAAAAAAA
AAAAAAAAAAATGAAAATATAGGTGGACAGGCCGGGCGCGGTGGC
TCACGCCTGTAATCCCAGCACTTTGGGAGGCCGAGGCGGGCGGAT
CACG

In some embodiments, the intronic domain that promotes trans-splicing primarily in the brain comprises or consists of sequences from the gene DBNL. In some embodiments, the sequences from gene DBNL comprise or consist of:

(SEQ ID NO: 147)
AGTGAGCTCATGCAGCATCCACTCTCCTTGGTGCCCATTACAGAT
GGTCACACTGAGGCTCGGGTAAGTTAAGCCACAAGGCTAATGATC
GACTGGCTCTGGTGCCCGTCTTTGGCCATGTGCCTAAAACTCAGT
CCTGGGCAGGGGATTAGGCTGAAGTGGCAGCATAGGGCTGAGCGG
GCAGTGGCTCTCCCTGCAGAAGGAGGAGGAGAACCGTCGGCTGGA
GGAAAAGCGGCGGGCCGAGGAGGCACAGCGGCAGCTGGAGCAGGA
GCGCCGGGAGCGTGAGCTGCGTGAGGCTGCACGCCGGGAGCAGCG
CTATCAGGAGCAGGGTGGCGAGGCCAGCCCCCAGAGGTGAGCCAG
AGGTGGAGGCTGGCCTGGGGGACCCACCCTGAGGCATTGCTGGGC
ACCTGCTCCATCCCATGGAGGCGAGGAGGACTAAGGGGTGTGGCA
TGAGAAGCTGTGACTGTGCCTCAGCTGTGCCCCACCCGGCCCTTC
CCAG

In some embodiments, the intronic domain that promotes trans-splicing primarily in the brain comprises or consists of sequences from the gene DBNL. In some embodiments, the sequences from gene DBNL comprise or consist of:

(SEQ ID NO: 148)
ACGTGGGAGCAGCAGCAAGAAGTGGTTTCAAGGAACCGAAATGAG
CAGGTAAGATGGGGGTGCTCTACTTGTTTGGACCTGTCCTGGCCA
CACGCAGAAGTCCCTGATCTCGGATTGAGGGCCCAGCCCAGACCT
GGGCAGAGGCTGCCCTGCAGTCAGCTGGGGCAGGTTGGAATCTGG
GCACCTCAAGAGGTGGCAGTAGAGAGGAAAGCCAAAGGCGGAAGC
GTCGGGCTTGGACCACACCTGGTCCTGGGGGAGGCCCTGGGAGCC
CCTTGGCTTCTGTGTTTTACTTCCTTTTTTAACGTTACTTTTTAT
TTTTAAATGACTTCTCTCCTGAGAACATGTTTTGCCTCCTGGCCC
CACACTCACCTTTGAGGGGCTACTGGGCCGACAGCTGGAGGGGCT
GTGATCTGGGGAGAGGTGGTGAAGGTTTTGCCCACTGCAGGGGTC
AACATGTGCTTCCCTCCAGGAGTCTGCCGTGCACCCGAGGGAGAT
TTTC

In some embodiments, the intronic domain that promotes trans-splicing primarily in the brain comprises or consists of sequences from the gene POLD2. In some embodiments, the sequences from gene POLD2 comprise or consist of:

(SEQ ID NO: 149)
GGTGAGACATCGGTATCCAGGCACGATAAATTTCCAAGTGGACAC
AATGTCTGGTGTCAACTACAGCTGTTCTCCTTCTTTTCCCAGTAT
CCTTTGGGTGCAGTGAGACACCAGGAGAGCTGCTGCTTTGGGGGA
TGGACAGGGGCAGCAGGAATGCCTTTGTGTTTTCGCAGTGAACCT
CCTTGGCCTGGGCGAAGCTGTGTGGACCAAGCAAGTCAGGAGTGT
GGCCATGTTTTCTGAGCAGGCTGCCCAGAGGGCCCACACTCTACT
GTCCCCACCATCAGCCAACAATGCCACCTTTGCCCGGGTGCCAGT
GGCAACCTACACCAACTCCTCACAACCCTTCCGGCTAGGAGAGCG
CAGCTTTAGCCGGCAGTATGCCCACATTTATGCCACCCGCCTCAT
CCAAATGAGACCCTTCCTGGAGAACCGGGCCCAGCAGCACTGGGG
TAAGTGAGAGTTTGGGAAGGTGCTTCCCCCACAGCATCCCTGAAC
TTAG

In some embodiments, the intronic domain that promotes trans-splicing primarily in the brain comprises or consists of sequences from the gene POLD2. In some embodiments, the sequences from gene POLD2 comprise or consist of:

(SEQ ID NO: 150)
TCAGCTCTCCTGGAGCTTATGGTCTAGTTGGAGCAGGCAGACAAT
ACATTTAAAATATACAGTTTGTTAGAAGGTAAATGTTGTAAACAA
CAATAACAGTTGAAGTACTGGGGAGAGTTGCAGTTGTAAATCAGA
TGGGCAGGGCACAAGGTAACATTTGAGTAAAGATGTAAGAACTTG
AAGGAGATGGGCAAGTGAGCTCTATAAGTATACGGGAGAGGGGCA
AGCAAGAGTTCAGAGGCCCCTTGCTGTGGGGAGGGATCCAAGGTG
GAGGAGTGGGAACCAGGAGGGGAGAGGACCAGTGGAGCAGATCTC
ATAGGCAGTTGTAAGGACTTGGGGCCTTATTCAATGAAATGAGGA
CACTTTGGAGAGTTTTGAACAGAGCAGTGACTGATTTATGTTTTG
GTTTTGGTTTAGTTCTATTATTATTTAATAATAGGCTTATTATTT
CACAGAAGTTTTATTTAATAAGGCAGACCTCTTGTCTGGAAATGA
GACA

In some embodiments, the intronic domain that promotes trans-splicing primarily in the brain comprises or consists of sequences from the gene OGDH. In some embodiments, the sequences from gene OGDH comprise or consist of:

(SEQ ID NO: 151)
TCTTTAAATTTGATTTATTTTAAATTGAGGTGGGATCTCACTGTG
TTGCCCAGGTTGGCCTCAAACTCCTAGGCTCAAGAGATCCTCCTA
CCTCAGCCTCCCAAGTAGCTGGAACTACAGGCATGTGCCACTGTA
CCCAGCACCTTAGGGAAATGTTTATGTTCTGAGCTGAGGCAGGCT
TCACATTGGAGTAAGAATTGCCCAGCATATCTCATTTCTGTTTAT
CCATGGAAACTGTATAAAGATCACAAGAAATGGATCCTTTCAGAC
TCCTAAGTTTATCATAACCTGAGTTTATCTCTTTTTTATAAAAGT
ACATTTATGCACAATAGTGCTTTTAAAATAAGTTATTCCAAGACT
TTTAAAAACTAATTTTGTCGTATGGGTTTATTGGTGCTGTGTTCC
TCTTTGGAAAAGGTTTGCATAACCTGTGACTCTTTTAACCCTCCC
CACAGCTCACTGGATCTGCTTAATGACTTGCTTGTGTTATTGCTT
CCAG

In some embodiments, the intronic domain that promotes trans-splicing primarily in the brain comprises or consists of sequences from the gene OGDH. In some embodiments, the sequences from gene OGDH comprise or consist of:

(SEQ ID NO: 152)
TGAGAATTAAGCTGTAAATGCTAATTTTAATGTAATTTTACTTTT
TTTTTACCCCTTCCCTCTTTTTTTTTCTTCTGTCCTTTTGTGTGT
GTCCTTCCCTCTCATCGTTGGCCACTCATAGATACGAGGGCACCA
TGTAGCACAGCTGGACCCCCTGGGGATTTTGGATGCTGATCTGGA
CTCCTCCGTGCCCGCTGACATTATCTCATCCACAGACAAACTTGG
TGAGGGTCTGAGAGCAGTCAGCTGCGTTGCTTGAGCTCCTCTCGC
TGTGCCACGACCTGTGGTAGCCAGGATGTCCAGGCAGGAGGGAAA
GGCTCTTAAGTTTCCTTTCATTCTGATCACTTGAAATTTATCGGT
ATTGTAGCCTTAGTGTGTTTTGGGCAAGTTATTTTTATCTTACTT
TTTCAGCCAGATTGTCTTAAGTCTCAAAATTTGAAAATAAAATTT
TGGACAGAAAGGTTTGTGGCACAGGGAAAAAGCTTGTCCACCTGG
GGCA

In some embodiments, the intronic domain that promotes trans-splicing primarily in the brain comprises or consists of sequences from the gene RFC2. In some embodiments, the sequences from gene RFC2 comprise or consist of:

(SEQ ID NO: 153)
GGCCCTCCAGGAACCGGCAAGACCACAAGCATTCTGTGCTTGGCC
CGGGCCCTGCTGGGCCCAGCACTCAAAGATGCCATGTTGGAACTC
AATGCTTCAAATGACAGGTAGGTCTTAGAGTCAGACCTGTAGGGG
GGCGGGTGCTCTGCATTGAGAATTGTTACAGAAACAACCTTTCTC
AGCATTGGGTTGTATGCGAAGCCTTGCCAGCTATGTGATTTCCTC
CACCCCTTCCCAGTGAAAGAGCGTTGCTCACTTGTCAACTTGCCG
GTTTATTTTTTTTGTTTTCATGGAAATTTTCAAAAACTGTTCTTC
TCAGCCAGGCATGGTGGCTCACACCTGTAATTCCAGCACTTTGGG
AGGCCGAGGCAGGTAGATCACCTGAGGTCAGGAATTCGAGACCAT
CCTAGCCAACATGGTGAAACCCCGTCTCCACTAAAAATACAAAAC
TTAGCTGGGCGTGGTGCTGGGTGCCTGTAATCCCAGCTACTCAGG
AGGT

In some embodiments, the intronic domain that promotes trans-splicing primarily in the brain comprises or consists of sequences from the gene RFC2. In some embodiments, the sequences from gene RFC2 comprise or consist of:

(SEQ ID NO: 154)
GCCTGTTGAGTGTCTCCATGTCTCATCCCACTGTCCGCTGAACTG
CTTTCTTGTGCACAGCGCTGCCTCCCTGGAGAGCCCCTTCCCATT
CCCCCGGCAAAGGGAAGTGTTTCACTCTAGTTGGGAGGCCTGGGG
TTGGTTGCTGTATTTTTTTTTATTTATTTATTTATTTTTTTTTCT
GAAACGGAGTCTCACTCTGTCACCCAGGCTAGAGTGCAGTGGTGC
AATCTCTGCTCACCGCAACCTCTCCCTCCCAGGTTCAAGCGATTC
TTGTACCTCAGCCTCCCGACTAGCTGGGATTACAGGTGCCTGCCA
TCACGCCCAGCTAAGTTTTGTATTTTTAGTAGAGACGGGGTTTCG
CCATGTTGGCCAGTCTGGTCTTGAACTCCTGACCTCAGGTGATCT
GCCCGCCTTGGCCTCCCAAAGTGCTGGGATTATAGGTGTGAGCCA
CTGTCTCCGGCCAGCTGTATTGATCTGTGTGTGCTGTGGGTGGAA
TGCA

In some embodiments, the intronic domain that promotes trans-splicing primarily in the brain comprises or consists of sequences from the gene NRCAM. In some embodiments, the sequences from gene NRCAM comprise or consist of:

(SEQ ID NO: 155)
GTAAGAAAGAAAGATTAAAATTGCTTTATAATGCTACATAATAAT
CCATTAAACTAAAAAGATCATGGGGGAAATCATGTAGATTGTAAT
ATTTTGTCTTCCCAGAGCCATGAGTTTGGACTTTCTCTTCAAATG
ACTTTTGATCCCTTTTTATATAATGGCAATCTAAACATTGTCAAT
GATCATAAGGCATTTGGAGGAGTAAATCAATCAGTTCTCTGAAAA
CATTAGTGAGCCTACTTTCTTAGACTCCACATAGGTTATGGATGC
TGAAGTAACACCTAAAACCAACTGGCAAGGAGAGAGTTGCAGGGA
AGGTTCATGGAGTTTTTCAAAGGAAGTAATGCATGAACTAAGTCT
TCATATTGTGATGTGTAACATTGCTTTCCCTATAAGGTAATGGTT
TATTCAATTTAGTGGGCCCATTAAATTTATATTTTCTTTACCTGA
CCATTTTCCCTTCAGTGACAAGAAAAAAAAAATCTGTGAGAGAAT
ACTC

In some embodiments, the intronic domain that promotes trans-splicing primarily in the brain comprises or consists of sequences from the gene NRCAM. In some embodiments, the sequences from gene NRCAM comprise or consist of:

(SEQ ID NO: 156)
TTAACAGATTTCTAAAAAATACTGAAAATGGCAACAGCCTTGCAA
GGACAGTTTAGCCTCTAAAAGCAGTTCCTTTGAAGAGAAAATACT
CAATTAGATGTATACATTCTGTTCTGTTTAAAACTCAGTTTTGTA
TTTTTCTCATTATACTTAATGTAAAATGTATCCTGTTAACAGTGA
ATACTTTAATTGGTTTGGGAATTTGTTTTTATTGAGAATATATAG
TGAAAATAACTGTGAACACAGTGTTCACCAGGGGTTTTTAGCATT
TAATGTGATGATTATTCAGATTTCTCTACTTGAATTTTTTAATGA
ATGAAGATTTTTCAAAGTTAATTATTCATATAATTCCTGATTAAT
AATTGACAGATCCTAATTTGGGGCCACATGTAAAATATATGAAAG
CTCACATAACGTGGCAATTTTTTACTCTGAGAGACTGCAGCAGTT
CCCATCTCCCCTTAACCTATTGCCACCAACACCATTGATTTGTTT
TCCA

In some embodiments, the intronic domain that promotes trans-splicing primarily in the brain comprises or consists of sequences from the gene MEST. In some embodiments, the sequences from gene MEST comprise or consist of:

(SEQ ID NO: 157)
TGGTACGTGCCTAGCAGTTTTTTAGAGGTTACCTAATAAAACAAG
CTAGGTTTTGTATCACACAAAAAATTATTTATAGAAAATATTTTC
TTGGGCTGGGTGAGGATGCTCACGTCTGTAATCCTAGCTTTTTGG
GAGGCTGAGGTGGGAGGACTGGTTGAGCCCAGGAGTTTGGCACCA
CTCTGGGCAACATAGTGAGAAAAATTTTTCTCTACAGATAATTTA
AAAGTTAGCCAAGTGTGGTGTTGCACACCTGTATGTAGTCTCAGC
TACTGAGGACACTGAGGTGGGAGGATCGCTTGGGCCAACGAGTTT
GAGGTTACAGTGAGCTATGACTGCACCACTGCACTCCAGCCTGAG
CAAGGGAGAGAGAGACCTTGCCTATTATAATAAAAAAAGGAAGAA
GAAAACATTTCTTTTCGGAGAGGAGTTAATTTTAGGGTGGGAGTA
GAACAGATGTGAGAGCTGTTAAGTATTTCATTCCTTTTCTCTTTT
CTAG

In some embodiments, the intronic domain that promotes trans-splicing primarily in the brain comprises or consists of sequences from the gene MEST. In some embodiments, the sequences from gene MEST comprise or consist of:

(SEQ ID NO: 158)
TGAGTCTCCCCGAGAGAAGTCTATGTTTTGTTAGTATCTCATCCT
GACAGTGGTAAACAGAATTGCCTGTTGAGGGCTATTGGCTCTAGC
CATTGTCTTTAACCTCGCTGGCTGTTCATCCAGGAAACAATAAAA
AGATTGAGTTGAGGAATCTTATTCTAACATAATTGTAGTATCAAG
ACATTCAGTTATACGTTCTCTTGATTTATACATAGTCTGTTTTCA
CTGTCATGACTGCTACATATTCCTTGGTGTACAAAAAGCTCTCAT
GTGAGTCAAAGGTTGAAAGTGGATATACCTACAAAGTGTTTGTTT
GTTTTAGTCTGATTGTAAAGCCAATTCATTGGTTTAGGCTGAGCA
GATGGCAAGGAGAGATTGAGAAGAGCTCATTGTGGAAAAGTGCCT
TCTCATTGGTTGCTATTCTCTCTAGGTACTTACTTGGAAGGTATC
AGAATATGAAATCTTGATTATGTCACATAAGAATGTTCCATACCT
TCCT

In some embodiments, the intronic domain that promotes trans-splicing primarily in the brain comprises or consists of sequences from the gene PTN. In some embodiments, the sequences from gene PTN comprise or consist of:

(SEQ ID NO: 159)
GTAAGTGAGAGGAACGTACGATTTCTCGGAGGGCTCTTCGATCGA
GTTTGCCTTCACCCTGTGCCACTTTTCTGGGTGCCAGCAGATGAT
GGTGGTAGGGGTGAGAGAGGCTGCCTGGTGTCTTTGCTGATAGGA
CTGCTTTCAAAACTGACTTGCTCCAGAGGCCAAGGGAGGCATATT
TTTGGGGTTTCAGGTTAGGTTAGTTCGGGTGTGCTTAGGTAAGTG
AAAGGAAAGAAGTTTTTGTGATGTTAAGCATGTATTTGGACCGTG
TTCCAGCTACCAGCTGGAGAGCAAGACAGGGTGGGCTGGAGAGTG
CATCTTTGCTTTTTGTAAAACCTGGAGCTTTAGTAGGTGTGTCTC
TGGCTGGGTAGGTGCAAAGAAAGTAAGGCAGCCTGAACAAGCAGC
TCTCATTTTCCCAGCCTCTCTCTGATTCTCTGATTGCCCCAGAGC
CTGTCAGCCCATGACTCTTAGCTGGCATTTCTAGGTAAGCTGTGG
GGGC

In some embodiments, the intronic domain that promotes trans-splicing primarily in the brain comprises or consists of sequences from the gene PTN. In some embodiments, the sequences from gene PTN comprise or consist of:

(SEQ ID NO: 160)
AAGGCACCAAGGAAACCAAAAAAGGAAAGAGCTCTCCCTAAAGGT
AAGTAGCACTTAATTGGGAACTTTGAGACGTCTGTCACTCTTTGT
GACAATTGAGAAAAAGTACTTTGTACATTTTGTGCTTTGCAGGTG
AGATGTGACTGACACCTTACTGTCTCTGTGTTTAGAGAGGGCTTT
CGTGGGTGGAAGAAGAGGCTGGTTTGAAGAGAGTGCTTTTCCATT
CTGAATATCATTAAAGGATTGTGGGGGGCCAAAAATGTAACCTAA
AATGCCATCAATTGTCCATTTAAAATATCCTATTTGGCAAAGTGG
CTTTGCACTCATCTGAAGAATAGATCATGGGTGGAGTTTGGAAAA
GAAATGTTGTGTTTCGTCTCTGCTAAAAACAAAACAAGGTACCAT
CTCATGGCCTCTTGGTTCAAATCCGTTTTCTTGCTCCGCAGGGAG
AGACTGCCCCTGGAGTATTCTGCTTTTAATAAGCTTCCCAATCAG
CTCT

In some embodiments, the intronic domain that promotes trans-splicing primarily in the brain comprises or consists of sequences from the gene SSBP1. In some embodiments, the sequences from gene SSBP1 comprise or consist of:

(SEQ ID NO: 161)
TTAGAAACCTCATCACATAATTTCATTTCTTTCCAAACTTGAAAC
CTTCTAAACTTGAAACCTTCTAAACTTGAAACCTTCCAAACTCAC
CACTTGGCGGCGTTGATCCATCCGCCCGCGCACAAAGAGAAGATC
GCGGAACTTCAGAGTTAAGGGGAGCCGTTTCCCGCAGCGCTAGCC
GGCAGTATTTCCAAGGCGCAAGTTGCGGAGTTTCTGTTTCCTTTT
TCCTCTGGCGAGCTTTGCGTTCCCTGTGCGCCGGAAGTGATCCCC
TGCGTGGCTGGGCTGCTCGGGTTAGATCGTCAGGTGAGGGAGGAA
GGGATAGCCAGCGCGAAGGAAGTGCTGGAGTCGTGTGTTTTGGCT
GCGCGTGATCCTGCGTGGGTCGGGAGGTGTTTCTGTGTAGGTGTC
TGGCCCTTTCATCAGTCGTGCGGAGGACCGCGTGATTTCCTTCCA
GTTCTCCTCGGTTTTCAGGTGGTGGCGCCATCTTCGGTAAAGGGT
GTCC

In some embodiments, the intronic domain that promotes trans-splicing primarily in the brain comprises or consists of sequences from the gene SSBP1. In some embodiments, the sequences from gene SSBP1 comprise or consist of:

(SEQ ID NO: 162)
TACGTTGTGGTCACAAAATTGGAGTGCATAAAAATTATGAATATA
GTTAGGAATTTAGGACTGGTGAGTTGTACTTTTGCCATAAGAGGT
AATGTTTTTTTCTTATTTTGTTTTTTTCCTTCAGGAAAAGCCTAA
AGATTAGACTGTAAGAAAAGAAAATAGAAGCCATGTTTCGAAGAC
CTGTATTACAGGTAGTCACTTGTCTGTATTAATACTGAGATGTAT
TACTATCAGCCACAGTGATCAGAAGACTTCTTTAGGCTTTTAACT
ACAGGGGCAAAAGACCTTTGAGCTCACCCACCCACCTCTATACTC
ATTTGATATGGAGGATTGGGGACGATTTTGCCAGTGTAAATTATG
TTCACTAAAACAAAAAAAAAGTTCAGGTTTATTCACACGTAATAG
GAATCATTAATTTTTAAAATGTGTGTGTGTTGCTTGAATTTAACC
AGTGAGAGGACATTAAAGGTCTTTAGTGACCTAAAGACTGTAGGA
ATCA

In some embodiments, the intronic domain that promotes trans-splicing primarily in the brain comprises or consists of sequences from the gene CNTNAP2. In some embodiments, the sequences from gene CNTNAP2 comprise or consist of:

(SEQ ID NO: 163)
ATTTCCCTAAAATATCAGTTTCATAAATTGCAAACATATGACCAT
AGTCCCCACTGAGACCAGAGTAATATGTTAATTAATGTTGGCATT
GACCACCCAGAATAGGACAGAGCATCTCTCTCCCTGACTTATGTT
CGGCCAGCAGTAGCTTTGCTGTCTTCAGAAAGTCTAGTGATCCAC
AGTCACACCCACAACACACATACACACTTTACAACTCATGGTCAG
CCTATCACATCAGGCTGTCTGGCTGTCAATCCCATGGTGGAAGCT
GCTTGTTATAGTTAGACAGCCAACTGCTGCTCAGTAGTAGGTCTT
ACACTCCAAGTAGATCCGTAGTGGTGCATCCAGTCACTGAATCCA
TGCTCTGCTGCTGTGTGTGAGTTTAGCTTAGGCTCAGGCTGTGCT
TCAAAACTTGTAGAACTATACTTTCATCAGTGGTCTGACATGGAT
GTTCATTTTATTCTGTGTTTTCCTCAGAGCCTGTCTTTCTATTTT
ACAG

In some embodiments, the intronic domain that promotes trans-splicing primarily in the brain comprises or consists of sequences from the gene CNTNAP2. In some embodiments, the sequences from gene CNTNAP2 comprise or consist of:

(SEQ ID NO: 164)
TCAGTGAAACCTATTTGACATTTGTTCCTGAAACTTATTGCAATT
TCCAGAGTTAATGTTGTGCTTTGTTTGGTTTTGCTGGTGTTACAC
GCTCAGGATTAGGTTTTAATTCAGATCTTCAAGACGCTTACTTGG
TTGTAGTGTGTGCTGATTGAATAAAGAAAACTTGGAAAGTTGTTG
AGAGAGCAAGGCATGGGTGAAGGAGAAAATTGGTTGAGATTGGAA
AAGCAAAATCTATTCCTTACCAATGCTAGTTTATTTTTAATAGAT
AAATTGCTCTTCTTTTAAGTGAATAATTTATGTTTAAAAACAAGG
AATGTTTATTTTAAAAACACAATTGTATCTGGTATCTTTTCTCTT
TATCAACATTCGTGTACCTTTTACTATTAGTAACAAGATTATACT
AATTTTAACTTTAAGGAATATTCCACTGCTGGAATCAGAACTTCA
AAATGATCTTGCTAAAATTATGCAAATGGTTGGAAAAGAAAACGA
AAGT

In some embodiments, the intronic domain that promotes trans-splicing primarily in the brain comprises or consists of sequences from the gene PTPRN2. In some embodiments, the sequences from gene PTPRN2 comprise or consist of:

(SEQ ID NO: 165)
GTATGACGCGCGGCCCGCCGGCAGGGCAGTGCGCGCTGAGCGCGC
GGCACAGGGGAGCGAGGCTTCGGTGCTCCTGAGACGCCCCTGGCG
CCGAGGGCCGCGTCTGTGTCGGCGCCGCAGGGGGGAGGGGAAGGG
AAGCGAGAGGCGGGAAGACTCCGTTAACGGCACGTGTTAATCGTG
AGCGATTTTCTAGCTTCAGAGGTATTAAAATGTGAGGGGGAATAG
GCATCTTCGAACTGTGGAAATAGGGCCATTATTTTTGATAAGGTT
CTTCAGTTGACAAAGAGCCTAAAGTGTATGTGCCACAGGATATTG
ATGCATAAAGCTCGGATGTTCAGTTGGATTCTCATCTGGCTGCAT
GGGATGAAAGAGGTAGCGAGCGCAGGCCCAGATACCACCCTGCGT
GATCTTGCAGGGAAGCCCGGAGACACTTAGCCCGTGGCTGTCTGT
ATTTTTAGGGCTATTTTGAAACTGACCCTCTTTAAAAAATTCAGT
TTTT

In some embodiments, the intronic domain that promotes trans-splicing primarily in the brain comprises or consists of sequences from the gene PTPRN2. In some embodiments, the sequences from gene PTPRN2 comprise or consist of:

(SEQ ID NO: 166)
GAAGTGCCTGCGCTGGGGTTCACATCAGCGCGACTCCGTGAGCCT
TTTGCTTTTCCTGCTCTAGACCCGGGTGTGCGCAGCTGCGACGGA
GTCTCACTTAGAGTGGAACCACTGAGCAGAACATGTAGTCGTGTC
GCTGCTTTTGTTGAAAAGTGTAATTTTTCTCTGGTTCCTTTTTTT
GAGGGTTTATGTAACATGGACATGCAAAAGACAGAAAGTAGGATA
GTTTTGGATTGTAACCTTTTAAAAAATTATTTGACGCTCAAGCCC
CTAAATAACACTAAGCACACACACCCGCACGGAGCTAACTTCAGT
GCGTTAACACTGGGAAGGTGGGAGGGGGCCGCGAAGCCGAGGGAA
CCCACCTGCGCTGTAAAATGTGCCCTCTTCGCAGCGCCCCCTTCT
CTGGTCAGACGTGGCCCTGGGTTCCTCAGTGCCTGAGGTTCGGTT
CACAATGTCCACACCTGTCTCTGTCCCCTCCCTCCGCTCCTCCCT
GCCA

In some embodiments, the intronic domain that promotes trans-splicing primarily in the brain comprises or consists of sequences from the gene FDFT1. In some embodiments, the sequences from gene FDFT1 comprise or consist of:

(SEQ ID NO: 167)
TAGAGAGGGGGCAGCTGAAGCTCCAGGAGTGTGTGGGGCTCCCTG
GGGCTGGATGGCGGTGGCGGGCAGGCGAGCTGGGCTGTGCTCGGG
TGTGTTACAGTAAAGACGCCCAGCTTGGCGCTGGCCCGGCCTTTT
CACGGTTTTAGGCTCTACAGAGAGCGGCTGCAGAGCTCACCCGGC
TGGCAGGAGCCACCGAGGCCGGACACGTGGGCGACTTATTGACCA
AGTGGGGAGGAAGCAGCCCCGCACTGCTCTCCCGACTGCGGACCA
CCGTTGGGCTCCTGCGCATCCTAAGCCCCACCGCCTCACCTCCAG
TCCCCACAGCGTTCGCGCTCCCAGCCGGGGTAAGCGGAAGAAAAC
AAAGGCCCGGCTCCATCAGGGCACCAATCCCGCTCGTCGGCCTCT
TTCTCGGCCTCCAATGAGCTTCTAGAGTGTTATCACGCCAGTCTC
CTTCCGCGACTGATTGGCCGGGGTCTTCCTAGTGTGAGCGGCCCT
GGCC

In some embodiments, the intronic domain that promotes trans-splicing primarily in the brain comprises or consists of sequences from the gene FDFT1. In some embodiments, the sequences from gene FDFT1 comprise or consist of:

(SEQ ID NO: 168)
TCCAGCCGGCCGGTGAGCGCCTGGGGACCGCAGAGGTGAGAGTCG
CGCCCGGGAGTCCGCCGCCTGCGCCAGGATGGAGTTCGTGAAATG
CCTTGGCCACCCCGAAGAGTTCTACAACCTGGTGCGCTTCCGGAT
CGGGGGCAAGCGGAAGGTGATGCCCAAGATGGACCAGGTGGGCCG
AGCCTCCCTGCTTGCCCGGGGCGGGGAAGGAGCTCGCTGGGCCGG
CCTCAGGGCCTGAGCGGCCGGGCCCGGATCTGGGGCAAGGGGCGC
GGCGAGCAGGGCCGACGCCTGGGTGTTCCCGTCCCCCTTTCCTCG
AGCCTTCCCCCTGTAGGGCCCGGGTGGACGCGGCCGTCCTGGCTG
ACCTGTCCCTGCCCCCGCAAGCCGCCCTGGGCATGAGCGACTTTT
GCGTGGTTCCCGGTGGTTGCGCTCCCCGTTTCGTCCCCTCCGTGA
GCATCGGCGCTTACCGGTATTTTAACCCGAGGGTTACACATCTGA
GGCA

In some embodiments, the intronic domain that promotes trans-splicing primarily in the brain comprises or consists of sequences from the gene DMTN. In some embodiments, the sequences from gene DMTN comprise or consist of:

(SEQ ID NO: 169)
CATGGTGGGGGACAGTGGGAATGGGGCCTTGAGGAATTGGAAGAG
AGCTGTCCCTTGCTCTGAGTTTTCTCAGGTGGAATGAGAGAGGGT
TTGGGACCAGGTGAAGGTGCTCACAGAGACGGCAGAGGTGTGGGG
CCCGCAGCCGCGTGCCCAGTGCAGCCAGCGCCCAGGGGCCTCTGA
ACCCTGCCGGAGCAACCCCTCTGGCCCTTCAGCACAAGGACTGTC
CCTGCCTTGCTGCCTGTCATTGTCCCGAGCTCCACACTGGGCTTC
CTGGAGTGGGGACTCTCCCCAGTGCTGCATACTCTTCAAGACTGG
CCTGCTCTCCAGCCCTGAATTAGGGTTCTCCTGGGGTCACCCCTA
AAGCCAATTCTAGAGCCTTATGAAAGGAAGTGGGGGGATGGGGTA
CTTGGTGGGGTTCACTGGGCACCCTAGCTCCTGAAGCTGTCCCCT
AACCCCCAGCCCCAGCACTGAGGACCCACCCTCAGGCCCCCTCCC
TCCA

In some embodiments, the intronic domain that promotes trans-splicing primarily in the brain comprises or consists of sequences from the gene DMTN. In some embodiments, the sequences from gene DMTN comprise or consist of:

(SEQ ID NO: 170)
TACCGAGCACCCTCCCCTCCTTCTCCCCTCCGCCCTTTCCCCTTG
ACTCCCCCTACTCTGCCCTACATGCACTGGCTTCAATAGGAGGAG
GCACCCCTCCTCCCAGGCCCAGCGATTCTACCCATTCCTCATCAC
CCACCTCGTTCCAGGCCCCTGGCCACAGCTGTCCTTCCTGGCATC
AGCTCATTCCAACTCTACCTCTGGGGACCCTTGCACCAGAAGACA
GCTCTCCTGGGGTGGGGCCTGGTGGGGGAGGGGCAATACAGGGGA
GGAACCCAGGTCGGGGTGCGGCGGGGATCAGGAATTCTAAAGGGC
TGAGGTGTCAAAGCCCCAAGTACCTGGTCCGCTGGAGGGACCGAG
AGAGATCATCTAGGGCTGGAGTGGGGCTGGGGCTTCCCCTCTGTC
TCTCAGACTCTGATTAGAGGGAAGTAAGGGTGGGGTTGAAGGTAC
AGAAAGCCGAGAGGCTGGGCTGAGGCCGGGCTGGTTGTGGAGCGA
TGGC

In some embodiments, the intronic domain that promotes trans-splicing primarily in the brain comprises or consists of sequences from the gene CCAR2. In some embodiments, the sequences from gene CCAR2 comprise or consist of:

(SEQ ID NO: 171)
TTTGAGCCCGGAACCAATGATCCGAAGCCTTCTTTCCTGATGGGA
TTCTTTAGCGAGGACACTACCGAGGCGCGACTTCCTGACGCGCAT
CCGATCCGGGAAGGCGCACTTCCGCTCCCCAGTTGCCGCGGGATG
GCAAGCCGGCGTGGGGGAAGGGAGAGGTCGGCGGAGACGAGGGAG
GGACCATATCCGGGAGAGCGGCAGCTTCCGACCAGTGGTCGCGCT
TCCGGAGAGGCGCTTCCGGTGGCGGCGGCAGCAGCGGCTGTGGTG
GTTCCGGGTGTCTTTGTCCCCCCGGTGTCGCTGCCCTGGCCCGCA
GGTGGGTTGGGGGGCCCCCTGCCGCCCCTCTGCCCCTTCGCCCCT
CCGCCCCTCCGCTGGCCGAGGGGCTGCGAGGGCCGGGCGGCGAAG
ATGCGTGGGGCGCGGAAGGGGCCGAGCCCCTCTTTGGACTGCAAG
TCCCGTCTCCCTGGCAACGGCCTCGGCCTTGGGGGCGGGCGGGAG
GAGG

In some embodiments, the intronic domain that promotes trans-splicing primarily in the brain comprises or consists of sequences from the gene CCAR2. In some embodiments, the sequences from gene CCAR2 comprise or consist of:

(SEQ ID NO: 172)
TGAGTAGCAGTTTTGTCACTACCTGGTTATGTGGCCCCGGGCAAG
TCTCTTGATCTCTCTGAGCATCAGTTTCCTTGTCTGTAAAACAGC
ATGACAGTATCATGCACCACTAGGTTATTGTCAGGGCAAACTGGA
ACAATGTAATTTCTTGTTTCATTTCTTTCTTCTCTATATTCTCTC
CATCCATTGCTTTCATTCATTCTTATTACCCACCTCCCTGTTTTC
CGAAATATCCTTACTTAAGATTCTCCACTTTTCCGGGTATAGATG
GAAATTTCCCTTATAAGCTGTTAATTTCTTTCTTTTTGGATTGAA
GCCTTTTCCCCACGACTCTGAAAGAGGACAGCGTTCCCAATGTCC
CAGTTTAAGCGCCAGCGGATCAACCCGCTTCCAGGGGGACGCAAC
TTCTCAGGTGATCACTGTTCTCCCTACCTGGCCTCATCCTGGGAA
GTATGTGCTCTGGAGTTAGTTTGGGGTTTCCCTGGTGGAGAGCAA
TTTG

In some embodiments, the intronic domain that promotes trans-splicing primarily in the brain comprises or consists of sequences from the gene GOLGA7. In some embodiments, the sequences from gene GOLGA7 comprise or consist of:

(SEQ ID NO: 173)
CCTGCTTTCTACTTCTGAACACAATGAGCAGTTTGTGTTTGAGGG
TTTGGGGAGGGGGTGTTGTTTTTTCTTTATGTTTTGTTTTTTGTT
TTGTGTATGAGTTGGGTGGCATAACCCACACGTAAGCTGCGACGT
CCAATCAAGAACGAAAATGACCCCTCACAATCAGTGATCGGGAAC
CACCTGCCACAATCACTAGGGAAGTGACACAGCCGGGAAACTCCG
CCCCTCACACCCCCGCGCGCTCGCGCCGGCCGCAGCCCCACAGCT
CGCCCCGGCCGCAGCCCCACAGCTCGCCCCGCCTCTCTCCTTCCG
GAAACAATCCCCGCGCGGGCGGAAGGCGGGGTCGGCCCTGGCCCC
GCCCACATCATTTCCCTTCCTCGGCCCAGCCCCACAGCGCGCAGC
TTCGGCAGGGTGGCTTTACGGCCGGAGGAGAGGGTGGGTCCCAGA
CCTGAGGTAGGAGGCCGAGCTGAGGGGCGGGGAAGAGGCTTTTTG
CGGC

In some embodiments, the intronic domain that promotes trans-splicing primarily in the brain comprises or consists of sequences from the gene GOLGA7. In some embodiments, the sequences from gene GOLGA7 comprise or consist of:

(SEQ ID NO: 174)
TTTGTGTTTCCCGGGCCGAACCGGGTTGTGGGGGGCGCGGGGCCT
GGGCCAGGCGGCAGCTAAGGCCCGCGGTGACAGCATGGGTGAAGG
GGAGCGGGGCAGAGGAGGCCTTGGGCTGTTTTCGGCGGCGGGTGG
GGGCGAGGGGCTGGCGGGTCAGAGTCCCGGGTCCAGGCCGGGGCT
CTGACTCGCGGTTGGTGTTCCCCCGACCCCGCAGCGCGGGGTGTC
CTGTCCTCGCCATGAGGCCGCAGCAGGCGCCGGTGTCCGGAAAGG
TGTTCATTCAGCGAGACTACAGCAGTGGCACACGCTGCCAGTTCC
AGACCAAGTTCCCTGCGGAGCTGGAGAACCGGGTACGCAACCTGG
CTCCCCACGCCTGCTCTGGCGAGGGAGAGAGACTCACCGGGGACG
GGAAGGGTGTGGGCGACAACCGGGCCTTTGAGGGTCCCGCAGTTC
TGGGGAGGTCCAGGCACAGAAGGGGCCTTGGCCAAACGTGTAAGA
AGAG

In some embodiments, the intronic domain that promotes trans-splicing primarily in the brain comprises or consists of sequences from the gene TPD52. In some embodiments, the sequences from gene TPD52 comprise or consist of:

(SEQ ID NO: 175)
GTAAAGTATATTTTTATGAAATGTAGTTGGTCTTATGCTTTATTT
TTCTTTATTTAGATGTTTAACTTGAACTGTTCTGAATTAGGGAAC
TAGAATTCCCCTTGAAACAAGCTGTGTGTTTAACACTCTATCAGT
TTTCAGCTACACTTCCTCTGATGTTGGGATGGGTGCAAGAGATCC
GTTAAATGTTTTGATTTTGTTTTTAAAAAAATTATTTCTATTTTA
TTTTGTAAAACCTTTGTTTCCTGTCTGAAATCTGTTCACAAAGTG
AGCACTACTTGTTAACAAACCTTCTTTTGTCCCGCCACCTCTGTC
TGCATTCGCTTTAGTTACATTCTCCTTTTCTTCATCCTTCCCATT
TTCACTTCCCCCATCCCACTGTGACATTTGGAGCCAACTATGATT
TAATTCTACTATCTGCTTTCATTAGCACTTGCAAGTGATATTACC
TCACCTAACCTCTCTAGAGAGTTAGAGGATTTTTAACAATCTGTG
CTTG

In some embodiments, the intronic domain that promotes trans-splicing primarily in the brain comprises or consists of sequences from the gene TPD52. In some embodiments, the sequences from gene TPD52 comprise or consist of:

(SEQ ID NO: 176)
GGTAACCTTGAACCTGAGTGGTCAGTGAACTGCATTGTCCTTGAG
TGGGCAGTTGCCTCCTTGTCTTACTAAGAGCCCTTCCCTGGCCTT
GCTTGTGTGTGTATGTGTGTATGTGTGTGTGTGCATGCTTCATTT
TTGAGGGGAGGCGATAAACACTGAATTAGACCCAAGTTCTATTCT
TGATTCTGCTGTGACCTACGGTAACTCATTTAACACCTATATGCC
TTACAGCTCCGGGTGAAAGCACTTTAGTTTTCAAAGTGCTGTAAA
AATGCAAGGTGGTATTGTTAGCTCTAGTAACTCCTGGGAATTAAT
TGGCTCCTGAATAAAAGTAAATTATGTTGGAATAACTTTGATCCC
CTCCACCTCAAAAAGTAGGTAATTCTTCATGGCGCCAAGAGAATA
AAGCTCCTGGGGAGCTAAGTTTTGTCAGCTTTTAATAATGTTGAA
GCACCACTATACTAACCTTATTTCTATCTCTTTTTAATGCTGCCT
CTTA

In some embodiments, the intronic domain that promotes trans-splicing primarily in the brain comprises or consists of sequences from the gene DCTN3. In some embodiments, the sequences from gene DCTN3 comprise or consist of:

(SEQ ID NO: 177)
GACCAGTGTGTGGAAATCACTGAGGAGTCCAAGGCTCTCCTGGAG
GAATACAACAAGACTGTATCCTTTCTGCTCAACTAGGACCCTAAT
GGGTGCTGTCCCTAAGCCCTGTCCTTTTGTTCCACCTCTAGCTCT
CTCTCATCCTATCCTACCCACCTATGTTTGCCTGTTCCCCACTTT
CCATGAACTTCCTCACTCAGATTCCTATTCTCACCTTGACCTACA
ACCAGACAATGCTTCTCTCCAAGCAATTCGTGCAGTGGGATGAGC
TACTTTGCCAGCTAGAGGCCGCCACGCAAGTGAAGCCAGCAGAGG
AGTGATAGCTGCTCCCCATCCCAAAGTGGGCCTGGGCAGTCAGGC
TCCAGGGCCCTATGCCAACCTGCCTTTGTTACAAGGCAGAGGAAG
CTTTGTATTTATTGGCTTCAAGGCCCACCTCTCTGTACTCTGGGC
TCTAAAGTTGGAGGTCAGGTTACCTGAGGTTTGCAATTTGCAACA
CCCA

In some embodiments, the intronic domain that promotes trans-splicing primarily in the brain comprises or consists of sequences from the gene DCTN3. In some embodiments, the sequences from gene DCTN3 comprise or consist of:

(SEQ ID NO: 178)
CAGCTTCAGGCCTTTCTGTTGTAACCTCAGCAGAAACTTTGGGGT
TTATCCTTTATCTTGCATATGCCCCAGGGAGGTAAAGGGACTTGC
CTAGGTTCATGTAC
AAACTTTCTGCTTCCAGAGCAGGACTTTATTGGGACACCATAAGG
TTGAGGCTGGGAGGTACCCAAGAATTCCTGGGGATGGTATGTCAT
TGACTGTGGGTAGAGGGGCTCTTTTTTCACTATTCCCAGGCCCTA
GTGTAAAGTTCAAAGAGAGTTCAGAAATAAGGACAGAAGAAGAGA
CATACACTAGGGGGAATGTTTACTTAATGTATGGCCACTCCCTTT
TCCATCCGTCTTTGGACTGCTGTTAGGGCACCCATCCTGGAGCCT
GAAAGTGCTCCCATAGCCATTTGCTCCTTTGGCTTTGGGGTTAAG
CGTCATCTTTTTTAGGCTCCATGGGGAGTGGGAGTCCGTGATGAG
GCAGGAAGTTTAGTGGAAGATGTGGGCTTTGCCCA

In some embodiments, the intronic domain that promotes trans-splicing primarily in the brain comprises or consists of sequences from the gene SPIN1. In some embodiments, the sequences from gene SPIN1 comprise or consist of:

(SEQ ID NO: 179)
TGGTGAAGACGTCTCTTCCAGGGAGTCACAGGAAGCAAATCCATC
CTTTTAAAGGGCTCGCGTCCCGCGGCCCTGAACCGGCGCGCTGGA
GCAGTAATGCGACGTTCTCCGAGGTTCAAGGCTCACGGCCCAGCC
CTGTGATCCAGTTTCCTCAGTCTTCGCGGTGTGCACCACTCAGGA
CGACTTGCGGCTTACGTCCAGGACAAAGGGGGCTTGTCCTTTCTT
CAACGTCCTACTGCGCAAAAACGAAGCCCTTCTCAGGCTGCCTGC
CTTCCAGGAAAAGCTGCGCCCCCTCAGGAACCTGTGCCCCGCGCG
CCTGCCGCCGCCCATTCCCGGCGCGCACCTGGCAGTCCCGGGCTC
GAGCCCCGCCCTGCCGCCAGCCAGGTGAGAGGGGCCAGGCCCCGC
CCTCGCCGGCCCCGCCCCGCCCCACTGCGCCTGCGCAGTCGGCCG
TCGGCGCCAGGCGCCGGGGCGGCGCGGCCGTCGCAGTTCGGCGGG
CGCA

In some embodiments, the intronic domain that promotes trans-splicing primarily in the brain comprises or consists of sequences from the gene SPIN1. In some embodiments, the sequences from gene SPIN1 comprise or consist of:

(SEQ ID NO: 180)
TGAGGCCCGCGCAGGGCGGGCGTGGGTCGTGAGGGAGTGCGGGCT
GCGGGCGGGCCAGCGGTGCGGCGGTCTGCCCGGGGGCGCAGGTGA
GCGCGGCCCGCGCGGGGCCCGGCGGAGCATTGTCCAGCGGGCGGC
GCCCGCATAGCGCCCCCTGCCGGCTGGGCCGCGGCGGGGACCCGG
GCTGGGCGGGCCTGGGCCTCGGGGCGGGCGCGCGCTCCTTTGTTC
GCCGGCCCCTACTCCTCCGTGCCCCCATCCCCGCCGCCCCCCGCC
AACATGGCCGCCACCCTCTCGTCCCCAGTTCCCCGGCCGCCGCCG
GCCCCTTAGCGCGCGCTTTGTTTGGAGCCGGGCGGGGAGGCCGGC
GGGCAGGGGGCGGCGGCGCTGCGCAGTCGGGCGCGGGGAGGGGGC
GCTGCGCCAGAATGCAGGCGGGGAGGGGGAACGTTGCATAATCCC
GGGCCGGCGACGGGGGCGGGGCGGGGGGAGGGGCGGGACCCCGGC
GCGA

In some embodiments, the intronic domain that promotes trans-splicing primarily in the brain comprises or consists of sequences from the gene SEMA4D. In some embodiments, the sequences from gene SEMA4D comprise or consist of:

(SEQ ID NO: 181)
GTAACGGGCTGGGCTGCCCTTCCCCTCTGTTCGTGGGTGGGTGGT
TGCACAGCTCCTCCCTCCTGTCTGCCCCAGTGCTGTCTTCCCCAG
CACATGCAAGCCAAGCTGATCCTGCGTCTAAGGAAGTCTAGAAGG
TTTAAACTCACAATAATTTGTTTTCAGTGCCTTTCTAGGAGTGTT
GCTGAGTTTAAGTGGATGCACAGTTTAAGGGCTTAAATAGCCTTG
TTTCCCATTATTTTCACAGCAAAGATTTCTGAAGACGTCTTTCTA
GAAATGACTGGAAAGTTTCAAGAGGCATAAGATACAGGTAAAACC
ACTTTATTCATGATCTGTAATTTCAGTTGAGCAGAGTTGATAATG
TGTAGAGGCCTCAGTTGGATCTGGCAGGTGGTTGGTTGCTTGGTC
CCTTGGTCCCTGATATCCTGTGGGGCAAAGCTTCAGACTTGGCTG
CCATCCCTTGGGCCCGGCATGGGGAGGTGGGTGCTGGGCACCCAC
AGTG

In some embodiments, the intronic domain that promotes trans-splicing primarily in the brain comprises or consists of sequences from the gene SEMA4D. In some embodiments, the sequences from gene SEMA4D comprise or consist of:

(SEQ ID NO: 182)
CAAAAAAAGAGCCGTGATCATATAAGCTCTGAAAGCAAAAGGCAA
ACAAAAAAGAAACCCTCCTCACCACAGAATATGCTGGAGTCTGAT
TCCAAGGTTTAACAATTACGTGGCTCCTGTGTTAATAGAATCATG
GAGGGATCAGCGTCGGCTCCTGAGCGTCTGCAGTCCTGCACGTGG
CTCTGGTTTTGCGATTCTGATTCTCTTGATCTAATTGTTGGTGCT
GCTACTGCACCCCACGATCCCTGTCTTGAGACTGATCTCCCACAC
TGCAAAGGAGTGACAGCCATGCTATGGAACCTTGCCACACCACGG
TGGACACCGTTCTGTTTGGCTGAAAACCAGCCATCCAAAAATAAA
GGAAGATGAACAGCTCTTTAGTGCTTGTAATTATGCCATATGTGT
TTCTGCTTTTTCCTTCTTTGCCTCATGAAGATGAATTTTAAAGGA
TACATATCTGGAGCTAATAGACAATTAGATAGTACTTTTTTGGTT
CACA

In some embodiments, the intronic domain that promotes trans-splicing primarily in the brain comprises or consists of sequences from the gene GSN. In some embodiments, the sequences from gene GSN comprise or consist of:

(SEQ ID NO: 183)
TGCTTAATGGTCCACTTTAAGGACCAGGGATGCACTTGGCATTTT
CGTTACTTCATTCGGAATGAGCTGGGAATCGCTCTTCCTCCTTAT
TCCCTTCCGTGGGAGGGAATCTCCCCTGCTTTGCATGCAGCCTAT
GTCACTCTTCGAGGGCTTTGCCTCCTGAAGTCCGTTCTCTCCAGG
GCTGAAATCTGCCTCCGCTGGTGCTAGTTGAGCATTGCTCGCTTG
CCCGTCCCATCTGAGCTCTTTGGCCCCAGCCCAGCTCAGGAGCTG
CCCTGGGTCTCCTCCCCGACCCTAGGCCTCTTCAGATCTCCCCTC
CGTGCTCCGGGCCTCAGTTTCCTCCAGGGGAAAGCAGAGGGCTTG
GTTTGGTGCCTTCCTGCGAGGTGAAGCGAGGGGTCCCCCTGGACG
AAGGCGAGCTTGGAGCCACCCGCCCAGCAGCCGACGGGAGGGCCT
GGCGCTCCTCCCCCACCCGCCTGCCCGGCCGCCCCGCCCCGAGGG
CGGC

In some embodiments, the intronic domain that promotes trans-splicing primarily in the brain comprises or consists of sequences from the gene GSN. In some embodiments, the sequences from gene GSN comprise or consist of:

(SEQ ID NO: 184)
TAGGGGAGGCGCGCGGCCCCGGCCCCGGGCATCTGGGGGTCCCGG
GCTGATTTGAGTCCTTCCTCTCCCTTCCTTCCCGGTCCTATCTCG
CCCGCGCCTGGAGCTCGGCCCGGCCGCGGGGGCATCGCTGGAAAA
GGGCTTCCTCGCTCACTTTTCCCGTCTTCCAAGGGACCAAAGAGT
GTGGATGGGAGTTGGGGGATGGGGAGACGCCGGAGCAGGAGAGGA
GAGGGGCCAGGCCTCCACCCCGTGATTCCACTGTGCTGCTAGCAC
CTGAGCTAATAACCTCGACCCCGCCCCCGGGATGGGGGAACTGGC
CCGGGGTCACCCCTGCACTTGGGTTGTCTGACCCGGGCATGCAGG
GGACCACCAGCTACGGTCTGGGCGGTGACTCTGCTGGGGAGTCCA
CTCAGAGGACCCTTGCCTCCTGGACGATACCTGTGCCTGCTCTCC
TCTCCAGCTGCTTCCTCTTTCTGGGGACTGTTAGAAAGTTCTTCC
TTAC

In some embodiments, the intronic domain that promotes trans-splicing primarily in the brain comprises or consists of sequences from the gene STXBP1. In some embodiments, the sequences from gene STXBP1 comprise or consist of:

(SEQ ID NO: 185)
GTCAGGTCACCAGCTCTCCACATAACTGTTTTTCCCTTCTGTTGT
GTAGCCCCGGTGATAAAGCTCTCTGCTCCCTGCTTGCATATGTAT
TGCATGAATGTAGTGAGTAATTCTGAGCCCAAACACAGCTTTTCT
GGACAGAAGCTGTCACAGCCAGGCTTTGGGTGTAAATCTTCCCCT
TCATCTTTAGACCTGACTTGAGGTTCCATATATCGGGAGGTGGGA
ACCTGGGGGTATGCAGTTGCCCATGAAGATTCATGGAGGGGCCTC
GCGCAGACAGCTGACGTCCGCTGTGTGGCCTCCCCTGCTTTGTGC
ATGCCAGCCTCCTTGCTACCATGTGTGTTCATGCGCGCTTGTGTG
TGTGTGTATGCATGCGCACACATGTGTGTGTGTCTTGCTTTCTCA
TACCTCATTGGCTGCATAAAATGTAGGCCTAAGTTGGCATGTTCC
TGTGACGTACCTTTGTTCAAGCAGTCAGCTGGCCTCTGTTCTCCC
ACAG

In some embodiments, the intronic domain that promotes trans-splicing primarily in the brain comprises or consists of sequences from the gene STXBP1. In some embodiments, the sequences from gene STXBP1 comprise or consist of:

(SEQ ID NO: 186)
TAAAAGCAGCTTATTACAGAAAAGAAACTCTTCCACTCTGAAGGG
CTTTCTTTGATTATTCCGTCACTCTTTTTCTTTATTTCTTCCTTT
TTAGTTTATTATTTATTTGATTTTGTTTCAACAAGAAAATGCTTG
CAGTTCTCAACACCGATTGAAAATGTGTCCAGTGCTGCCCTCACA
GCCCTGCCATGAACCCTCTGGGCTCTGACCTGCTGGCTCACCCAT
GCCTTGGGTTCCGCACTGCTTAGTCAGACGCCTGCAGGCTGCTGG
GTGGAAACTAAAAATGATAATGACTGCTGTGAACAGTTGTCAGAG
AGGGAGCTGAAGTTTTGGGGGATGCCTGAGGCACAGTGCCCAACC
CTGCTGGTCTGGGATGAATTTGTGGCCCCCACAAGGAATGTAGAG
TGTCCTGGTTGTGCTGGGTGTGGGGGTCTGAGAGCGAGGCCAGTG
GACATTCAGGGAACACCATGTCCAGGAAGGCAGGCGGTGGTGGAG
ATGG

In some embodiments, the intronic domain that promotes trans-splicing primarily in the brain comprises or consists of sequences from the gene FAM102A. In some embodiments, the sequences from gene FAM102A comprise or consist of:

(SEQ ID NO: 187)
CTGGGCTTCGCTGACTTGAACCTGGCCGAGTTTGCGGGCTCGGGC
TCCACGGTGCGCTGCTGCCTGCTCGAGGGATATGACACGAAGAAC
ACTCGCCAGGACAACTCCATCCTTAAGGTACCAGGGATCCTGCCA
CCTCTGCCACCCTGACCACGGGATGGGGACAAACCAGGCTGCTCA
TCAGAATCCTCTGGAGGAGGGTTGTTAAAAATACAGGCCTTACCT
GAATAGGTAGACATCGGGAAGGGGCGAGCCCTGGAATCAGGAAAC
CACTATCAGAGGGAGGCCTGGCTCCGCTGTTGGCCTCAGTTTCCC
CATCAAGTTCAGAGAGGGGGCCCTGACAGTCTCCAGGGTTTGGCA
AGCACCTGCAGTGGGTTCACGTCATGCTGATTGCATGCCCTCTCT
TCTCTTGTGGTCCCCTGACAGCAGCCCCACGAGCTGTGGTATCTT
TGATTAGTCCCAGCTTCAAATGGGAGGGAGTTAAGGCACAGACGT
TAAG

In some embodiments, the intronic domain that promotes trans-splicing primarily in the brain comprises or consists of sequences from the gene FAM102A. In some embodiments, the sequences from gene FAM102A comprise or consist of:

(SEQ ID NO: 188)
ACCACCTCGTCTCAATCTCACTTTCCCCTCCAGGTCTAGCAGACT
TGGCCTCCATCCACAGGGCCTTCTTAGCGCATGCCCTCTTCGCAC
TTACTCCCACTTCCCTCCCCATCCCTCGATCCTCTGTCCAGAGGT
CCCAAGGGCCTGTTCAGACCCTGAACTCTGCCCAGCAGCCCCTCA
CTCTGTCCTCTACCAGGGCGCCTTCCCTGGGGCTCCTGCTCATCC
TGCAGTGCCCCTCCCCTCCCAACCCCTGTGGGGGTGTCCCATCAC
TTTCCCCAGCACCCACTTCCCACAGGCCTTCCCTCCTCCCCTGCC
TGCTGACCCGGCAGCTCCCGACAGCACGGCCTTGTTTCCCAGGGA
ACAATGCATCTGTGTCACGGACAACAATGTCCTTTCATGTCAGGC
GCACTGGCCCCGGTGCACGCCGGGACGTGGCACAAAGGGTGCTTT
GTGCAGCTCAGGGATGTGAGTTCCTGGCTTTGCCATGTTGGTGCT
GCCG

In some embodiments, the intronic domain that promotes trans-splicing primarily in the brain comprises or consists of sequences from the gene PTGES2. In some embodiments, the sequences from gene PTGES2 comprise or consist of:

(SEQ ID NO: 189)
GTACGTGTTACAGACGGCCACGCCGCCCTTTAGGCGGTCAAGGTG
GGGCGAGGAGACGTTCGCCCCCCTGCAGTCGGCCGGGTCACTACC
CAAGAGCCTTTGGAGGCGGAAGCATGGAACGGTCTGCAAACGTTC
CCGAGCGGGCCTCTGCGGCTCTGGCGGGCGTTTCGAACTTGGGCG
CCGGGCACACGCCCAGTCCCGAGAGCGCTGAGGGTTCCCTTAGCG
TCGCCCTCACCCCGGCCAACCCGCGGGGCGCCAGAGTCCTGGCCC
TTTAAACGCCGCGCGTGCCTCGGCGTCTTCGTTTCGCGCGCCCGC
CCGCGGCGCCGGCGGAGCGAACATGGACCCGGCTGCGCGGGTGGT
GCGGGCGCTGTGGCCTGGTGGGTGCGCCTTGGCCTGGAGGCTGGG
AGGCCGCCCCCAGCCGCTGCTACCCACGCAGAGCCGGGCTGGCTT
CGCGGGGGCGGCGGGCGGCCCGAGCCCCGTGGCTGCAGCTCGTAA
GGGG

In some embodiments, the intronic domain that promotes trans-splicing primarily in the brain comprises or consists of sequences from the gene PTGES2. In some embodiments, the sequences from gene PTGES2 comprise or consist of:

(SEQ ID NO: 190)
CCGAAGCCGTCGTCCTCGCCTTCCGCTCTCGTCTTCACCGGCATA
CTCAGGTCCCCGCTGGGGACTGACACTGCGGGCTCGGTCGCCGCA
AGGACTGGAAGGAAGGTGGAAGATGCGGGGCGAAAGCGGAGCGAG
AAGAGTGGGACGCGGCGGAGCAGCAGAGCCGGGACTCAGAGCTCC
CCCCTCGCCCCGCCCCTCTCCCCGCCCACTCTGCGAGTCCCGCCC
CCTCTCGGGCGCCGGGCGGGGCCATCCCGGGGCTGTCCGCGGAGA
CGCCTATGCGGTGGAGGCTCCGGGCTTCAGCTAGGGCGGGGGAGC
CCAGCAGAAGGACCGAGCAGCCTGGCACCCCACTTTGCCATCCTC
TCCCTGGAAATCTCGGGGTCGGCGGGCCGGCCGCTTCGCGTGGGC
GAAATCAGAGACACGTGGTTTCCAAGGCCCCTTCGGGTTCGGGAA
AATTTTATGGTTCGGGTCACAGTAGGAAGCGGACAATGAGGCGGG
AGGG

In some embodiments, the intronic domain that promotes trans-splicing primarily in the brain comprises or consists of sequences from the gene DNM1. In some embodiments, the sequences from gene DNM1 comprise or consist of:

(SEQ ID NO: 191)
ATGAAGCAAATTCTGGGACACTGGGAAACGTAGAATTGAACAGGA
TGGGTGGATGAGGTCCTGGCCTTGTCCTGGCCTGGGTCAGCTCCT
TAGGAGAACCCAAGGCTGTGGACAAAGACGGCACTGGGAGAGAGC
AGGCAGCCTCAGTGAGGCCGAGGAGCAGGGGAGCATCCCTGGGAA
GAGGATCTGAACTCAATAGGGGTCTTGTACGGAGCAGGGGCTTGG
TCCCTCGTACCTCTGGCCATACCTATGGAGCCCAGGGGATGCTTG
GCAGCACCTGGGAGGTGCCAACCCCGGGTGGCAAGGGAGGGCCGG
TCCCACGCTCACATTGTCTTCTGTTCTCTCTCTCTCTTTATCTGT
GTCGATGTCTCTCTCTCTTCCCCCGTGCCCGTGCCATCCTCTCCA
CCCCTGGATTCCTGTCTCTGCTTGGCTTTCACCCACTTCTCCTCC
CCACCCACGGCTGCTCCTCCTCCTGTCCCCACCTCCTCCCCGGGT
GCAG

In some embodiments, the intronic domain that promotes trans-splicing primarily in the brain comprises or consists of sequences from the gene DNM1. In some embodiments, the sequences from gene DNM1 comprise or consist of:

(SEQ ID NO: 192)
TATGACGGCCGCCTGGGCGGGGCTGGGCCTGGCCGTCCATTCCTT
GTGGCCACAGCCTCCCGTGGGCAGAAGGATCTGCTGAGCCGGCCT
CACGGCTACCCGCAGGGACCCAGCCCTAGTGTTTCCTGCCAGTTT
CTAACCCTGGGTACTTGCACTCATGACCCCTCCAGGCCCCCATCC
CAGAAGACTTGACTCCAACCCAAGCCTCCTTGGTGGCACCTATGC
TAGTGATGAAGATGATGTTAAGGAGATGGCAGCTGTTTACTGAGC
ACCTACTATGTGCCAAGCACACGCTAAGTGCTTGCCCTTACTATC
TGACTCAGTCCTCTCAACCACCCTAAGACGTGGGTAGTGTTGTTA
TTCCCATTTTGCAGATGGCAAAACAGAGTCTCAGAAAAGAGAAGC
AGAGTGTGATTCAGTTTTAGGAAGGACAGAGGAAGGGGTCTGAGG
TCAGGGCCTCCTGGGCAGGGGGAGCTGTCCTAGTTCCTCAAAACC
AATT

In some embodiments, the intronic domain that promotes trans-splicing primarily in the brain comprises or consists of sequences from the gene URM1. In some embodiments, the sequences from gene URM1 comprise or consist of:

(SEQ ID NO: 193)
CTGGAGAGATAAAGGTGGAGGGGGCTCTTCAAGGTCCTCTCAGAA
GTCCAGCCATGGCAGTTGGAGACTGGCTTCCAGGACTTGTGGGTA
GGGGCCTGTCTCCTAACCCTTTAGATGCGACAGTCCTCCCGCCGT
CTGCCTTGAATCCCCCCACTATAGGTAAATCCCATTGCCTCTTCT
CCAGGACTTCAGCAGTGCCAGGTGCAGCTTTCTCTGAAGATCCAC
CTGGCAAGTCACTGGCCTGTCCTAACCTGTTTCCTCACCTTTGGA
ATCTACAAGTTCATGGGCTATCACAGCCTGGTCTAAAAGAAGCCC
TCTAAACACCGTGTGTATTTCCTTGTGGGCCAGGCAAGGCTCTGG
GGGTGGGCAGGTGCTATTTGGGTTTGACAAGAGTTTGTTCTGTGC
ATTCCTTTCCAGGGGACATCCGGAACCTGCTCATCTGGATCAAGA
AGAATTTGCTAAAAGAGCGGCCAGAGTTGTTCATCCAGGGAGACA
GCGT

In some embodiments, the intronic domain that promotes trans-splicing primarily in the brain comprises or consists of sequences from the gene URM1. In some embodiments, the sequences from gene URM1 comprise or consist of:

(SEQ ID NO: 194)
CGGCCAGGAATTCTGGTGCTGATTAACGATGCCGACTGGGAGCTA
CTGGTCAGTACCTTGGGGGACATCCCTCCCCCAGCCCCTGCCCTT
GCTGCTTCAGTGGGAAAGCGTTGGGCCTCTCCTCAGGCACATATA
GAGTGGCTGGGTAATCCTCCGCCCCACTCCAGCCCCACACTGAGG
CTGCTGGAGTCTCCCACTCCAGGTGAGGAAGGGATGGGTAGCTGG
GGACATGGGAGTACTCCTCCATCCTGAAGATTTGCAGGTTGAGGT
ATAGGATACCCATTTTTCTGGTAGAGTCTGTCTCTTCCAGAGCAG
CCAGTCCTCAGCCCCTGTTCTCCCAACTCCTGGGGTGGACCTGGG
AAGGTGGCCCTGAGGGTCTCCCGCTCCCCTCTCTCCCGCACCAGG
GTGAGCTGGACTACCAGCTTCAGGACCAGGACAGCGTCCTCTTCA
TCTCCACTCTGCACGGCGGCTGAGGGCCCTTCTCTGGGCCTGGGC
ACCC

In some embodiments, the intronic domain that promotes trans-splicing primarily in the brain comprises or consists of sequences from the gene SH3GLB2. In some embodiments, the sequences from gene SH3GLB2 comprise or consist of:

(SEQ ID NO: 195)
GTGAGTAGCCGGGGCTGGAGTGGGGCTCCTGGGGCTGGGGAGGGG
AGTTTGCCTCTGGGGTCCCTTCTGGGACTCCCACCTGCTAAGATG
TATCCCTCCTCTCCAGATTTCCCGGCACCTTCGTGGGCACCACAG
AGCCCGCCTCCCCACCCCTGAGCAGCACCTCACCCACCACTGCTG
CGGCCACTATGCCTGTGGTGCCCTCTGTGGCCAGCCTGGCCCCTC
CGGGGGAGGCCTCGCTCTGCCTGGAAGAGGTGGCCCCCCCTGCCA
GTGGGACCCGCAAAGCTCGGGTGCTCTATGACTACGAGGCAGCCG
ACAGCAGTGAGCTGGCCCTGCTGGCTGATGAGGTGGGCCCGGTGC
CGCAAGGTGGGGCAGGAATGGGCTGGGTGAGGAGCAGCCTGGGCA
CTGGGATCAGGCACATGGGGCCTGGAGGGGCCCTGTTGGCAAAGT
GGGGACCAGCAGGTTAAAGAGGGCATATCCAGCTCTGAGGAAAGG
ATCC

In some embodiments, the intronic domain that promotes trans-splicing primarily in the brain comprises or consists of sequences from the gene SH3GLB2. In some embodiments, the sequences from gene SH3GLB2 comprise or consist of:

(SEQ ID NO: 196)
GCCGGAGCAAGGTCTGGTAGAGGACGGGGATGGGGACGGAATTTT
TTGTTCTGAGGGCGGGAGGTTAGAAGTCAGCCTCTGGCCTCTTCC
CCGCAGGTGAACCACCTGCGCTGCCTCCACGAGTTCGTCAAGTCT
CAGACAACCTACTACGCACAGTGCTACCGCCACATGCTGGACTTG
CAGAAGCAGCTGGGCAGGTGCGGGGCCAGCACTGCTGAACCCACT
GGGGCCCCCTGGCTCCCATACCCTGTCCGTGGGCAGCATGAGCTC
CTCTGGGCAAGTCCTGTGACACAAAGCTGGGTTGGAGGGCTCTGC
TGGAACCCACTCTGGGTGCGGGCATCTCATGTGTATGAGGGTGGA
GTGGCTGGCCTTACTGACGGCCAAGTCCCCTGGAGCCCCTGAGGG
TTTCTAGGGAATGCCTGGGTGGGGAGGATGGCAGGGACCAGGGGG
TGGGGCCATCTCACCCTTCCCTTGCTTTGCTTTTCCCTTTGTGCT
GCCA

In some embodiments, the intronic domain that promotes trans-splicing primarily in the brain comprises or consists of sequences from the gene AIF1L. In some embodiments, the sequences from gene AIF1L comprise or consist of:

(SEQ ID NO: 197)
CTAAGCCCCTCCAGCCTCTGTCATGCGTGGGCACACACTTTTGCA
AAGCTGCCTGATCGTTGCAGGGGTGAGGATGGAGAAAATAATGGA
CCGAATGGCAGGCCTGTCCATTCATTCAATGGATGTTTTGTTGTT
GTTGTTGTTGCTTTTTTTTTTTTTTTAGAGACAGGGTCTGGCTGG
AGTGCAGTGGTGCAGTCAAAGCTCATTGCAGCCTTCACCTCCTGG
ATTCAAGTGATCCTCCAGCCTCAGCCTCCCAAGTAGCTGGGACTA
CAGGCACGCACCACCACACCCAGCTAATTTTTTAATTTTTTGTAG
AGATGGGATCTCCCTATGTTGCTCAGGCTGACTTCAAACTTCTGG
CCTCAAGTGATCCTCCCTCCTCGGGCTCCTCAACAGATGTTTATT
GAGTGTCTACGATATGCTGGGCACTTCGACGCTAAACCTGTGGCT
GGGGCCCTGCCTCCTCCGAGCTGCCTCCTGATACCCACTTCCTCC
TCAG

In some embodiments, the intronic domain that promotes trans-splicing primarily in the brain comprises or consists of sequences from the gene AIF1L. In some embodiments, the sequences from gene AIF1L comprise or consist of:

(SEQ ID NO: 198)
CAAGTTAGTTAACCTGAGTCTCAACTCCTCCACTCATCCTGCACA
TGTTTATTGAGCGTCTGCTATGTGGAACCTACATTCTAGCCGGGG
AGGCAGAGAGGGTGTAGTAACCTCAGTAAGCAGTGGCAGTGGAAA
TCATGTTTGGAGGTGACCCATCCTAAGGAGCTGGATAAGGGCGAT
TGGAAGTCGGGGCAGCTGAAGTGGGCAGGCAGCAGTATAAAACAG
GGTGGACAGGGCTGGGCATGGTGGCTCACACCTATAATCCTAGCA
CTTTGGGAGGCCGAGGCAGGCAGATCACTTGAGCCCAGGAGTTTA
AGACCAGCCTGGGCAACAGAGGGAGACCCCATCTCTACAAAAAAT
ACAAACATTAGCTGGGTGTGGTGGCATACACCCGTAGTTCTAGCT
GCTCTGGAGGGCTGAGGTGGGAGGATTGTTTGAGCCCAGGCAGGT
CGAGGCTGCAGTGAGCTGTGATCGTGCCAACAAAGAATAAATAGG
GTCG

In some embodiments, the intronic domain that promotes trans-splicing primarily in the brain comprises or consists of sequences from the gene SURF4. In some embodiments, the sequences from gene SURF4 comprise or consist of:

(SEQ ID NO: 199)
GTGAGTTGTGTAATTATTTCATTATGTATTACAACATAATAATAG
AAATGAACTGCACACTAAGTGTAATGTGCTTGAATCATCCCAAAA
CCATCCTCCCGCCTCCATCTGTGGAAAAATTGTCTTCCACAAAAC
TGGTCCCTTGTGCCAAAAAGGTTGGGGACTGCTATTTTTTTTTTT
CTTTCTGAGACAGAGTTTTGCTCTTGTTGCCCAGGCTGGAGTCCA
ATGGCGCTATCTTGGCTCACCACAACCTCTGCCTCCTGGGTTCAA
GCGATTCTCCAGCCTCAGCCTCCCGAGTAGCTGTGATTACAGGCA
TGCGCCACCACACCTGGCTAATTTTGTATTTTTAGTAGAGACAGT
TTCTCCATGTTGGTCAGGCTGGTCTTGAACTCCCGATCTCAGGTG
ATCCGCCCGCCTTGGCCTCCCAAAGTGTTGGGGATTACAGGCGTG
AGCCACCGAGCCCGGCCAGGGACTGCTGTTCTAAGCCACAGCGGG
CTGA

In some embodiments, the intronic domain that promotes trans-splicing primarily in the brain comprises or consists of sequences from the gene SURF4. In some embodiments, the sequences from gene SURF4 comprise or consist of:

(SEQ ID NO: 200)
TAGAGCCTGTGATTTGAGCCAAATCTGGAGAGATGGCCACCAAGC
CAGAACTCTGTGCAGAGACCAAGTGCTATGGGGCAGGGGGCTGGG
CCGGGGATTGCTGGAGGCTGACATTCATCAGAGGATTGGGGGAGG
GAGGGCAGGACCCAGAGGAGGAGATATGAGGATGGAAGCCTGAGC
CTAGGGACTCTCGGAGCCCATAAAAGTCTTAGGAGTCTGTAATCC
AGCCTTGTGAGCAGCCTGCTCATGACACTGGATATTCTAAGCCAG
GGGTCCCCAACCTAGTCCGTGGCCTGTTAGGAACCAGGCTGCACA
GCAGGAGGTGAGCAGCAGGCAGTGGAGCAAAGCTTCATCTGTATT
TACAGCCGCTCCCCATGGCTCGCATTACCACCCAAGCTCCACCTC
CTGTCAGCTCAGCAGCAGCATTAGATTCTCATGGGAGTGTGAACC
CTATTGTGAACAGTGCATGTGAGGGATCCAGGTTGCATGCTCCTT
ATGA

In some embodiments, the intronic domain that promotes trans-splicing primarily in the brain comprises or consists of sequences from the gene ABCA2. In some embodiments, the sequences from gene ABCA2 comprise or consist of:

(SEQ ID NO: 201)
GCCAGCCTTCTACACAGCGGCGCCCCTGACGTCTGCCGGCATCCT
GCCTGTCATGCAATCGCTGTGCCCGGACGGCCAGCGAGACGAGTT
CGGCTTCCTGCAGTACGCCAACTCCACGTGAGTGCCCACCCGCCC
CTCGGAGCACCCACCCCTCCCCAGCCCATCCAGTGAGTGACATCC
GCCATGGTGCAGGGTCACGCAGCTGCTTGAGCGCCTGGACCGCGT
GGTGGAGGAAGGCAACCTGTTTGACCCAGCGCGGCCCAGCCTGGG
CTCAGAGCTCGAGGCCCTACGCCAGCATCTGGAGGCCCTCAGTGC
GGGCCCGGGCACCTCGGGGAGCCACCTGGACAGATCCACAGGTGT
GCCCTGTGCAGGGAAGTTGGGGGGCAGGGCTGCGGGCAAAGCTCT
GCTGTCACTGGGCTGAAGCCCACATCCACTCTGGGCCCCTCCACC
TGCACTGAGTTCCCAGCCACGCACAGGCTGCAGTGGCAGAGCTGT
GGTG

In some embodiments, the intronic domain that promotes trans-splicing primarily in the brain comprises or consists of sequences from the gene ABCA2. In some embodiments, the sequences from gene ABCA2 comprise or consist of:

(SEQ ID NO: 202)
CGGCTTCTGGGCTGAGTCTGGGCTGAGGCTGGGCTGGCCTGGGCT
GGGGCTGGAGCGGCCTGGCTACTCTCCCTTCATACTCCTTCAGGC
TCTTGCACTCACTCTGCTAACCAGCTGGCCAGAGGTCAGAGGTCA
CTGCCCAGAGGGGGTCCTTTGTCCTCCCCTGGGCTCTGTAGGCAG
TGCCACACCTGAAACGTTTTCGGGGGGAGAGTGCCAGGGCATGTG
GGCCTGCACTGGCTGAAAGGAAAGGAATGCTCTGGGAGTTATGGC
AGAAGGGGTGGTGAGTGGCTCTTTGACCTCTGACCTGCTGGAGTC
ATTCGCCCCAGAACGCTTGGCCTGTAGCCCAGCTGGGGCCAGGGC
TCTGTGCCGCCTTCTCCCTAACGCTGCTTCTCCTTCCTGACTTTC
TCTAAATTTCTCCTCTCGGCCTCTGTCTCCCTCCCCTTCTGGATT
CCTCTCTCTCTCTCTCCCCTTTCACCCCCGACCCTGCCCTCTCCC
GTGG

In some embodiments, the intronic domain that promotes trans-splicing primarily in the brain comprises or consists of sequences from the gene GPM6B. In some embodiments, the sequences from gene GPM6B comprise or consist of:

(SEQ ID NO: 203)
GTACTTCCTTCTAATTCACCCCTCCCCCAGTTATTTCATTATCCT
CTCGTGCATATGTCTGGACCTAATTATTACATGCGCATCCTTTTA
ATTGTCATGATCTGTTTCATCCCTTCTATTAGAGTAATTTTATAC
TTTTCCCTTCAATATGCTATTTTCTGGGTGGAAACTTCAGGGAGG
CTTTTAGCTTGCTAGGTATCAAAATTTCGTTATATTTTCATGTTA
AGTGTGTAGTGAAAGAAGCTTTTCTTAATATGAAACTACTTTATA
ATGCAGTTCAATCAAAAAAGACTTCTGGATCTCCGCTACAAAAAG
ATTGTATTTTTTCTTTATTTTCAGTTTTATAACATCCAACTTTCC
ATTCAAAATAATATTGCACACATTGTATAATTCAATTAATTTGCA
CATCCAACTCCTCAAATCTGGTCTTTTTAAAATGACCAACTGCTT
AAACTTCATCTGTATTGAAAAATACAACATTAGATCCAGCCATAT
TTTA

In some embodiments, the intronic domain that promotes trans-splicing primarily in the brain comprises or consists of sequences from the gene GPM6B. In some embodiments, the sequences from gene GPM6B comprise or consist of:

(SEQ ID NO: 204)
GGCAATTGCTATTCTCTGTGAGCCAACCTGTTACGAAAAATAAAG
GTAACTTTTGAGAAATTAATATGAAGTTATAATTCAGTGGTATAT
GGAATACATCCAGAATCTATCCCATGTTAAGTAGCATGTTCTGCA
TTTTCAGAACGGCTATTTAGAAAGAGCACCTCTTGAGAGGCTGCC
ACTGATTAGCTCTGTGACCTTGGATGAGTGAGATCACCTCTTGGG
GTCTCCTGTCCTTGTTAGTTAAGAGTGACCCGAAAGGCCCTGACA
TTGCTTCGGGCACTCCAATTCTAGATCCTCTAGCTCTGGGGAATG
TGGCTGGCTCTTACTTCCCCTGCTCCCGTGCTGGCTTTTCCTTCT
TGCTACCAATGCCGGGGCGAGGGTCCCCACAATGTGTTCCACAGG
TCAGTATTGGGCCTGAAAATGCACAATCCTTCCACTTAGGGGACA
CACATCTACCTGTTTCAGTGATGATTGTCCTTCCTCTTGTTTTGG
CCTA

In some embodiments, the intronic domain that promotes trans-splicing primarily in the brain comprises or consists of sequences from the gene MIDI IP1. In some embodiments, the sequences from gene MIDI IP1 comprise or consist of:

(SEQ ID NO: 205)
CTCAGACAAGGCTTTTACCGGAAATTTAGCATATAGGATAGAAAG
CAGAGACCAAAGACCTCTTGCTTTGAGGCTGTTGGCGTGCTACAA
GGACTCGAAAGTTATTTGTTCAACGGATGACTTCTGTTTTCTGGA
GGCAAACTGAATTCCGGGCAGGTGGCTCAGCACCTCCAGCCTGTT
CCAGATCGGGACAGGGAAGGTGCCTGGGGCAATCAGTGAGGTTTC
CAACCTTCCTTAGGCCTGGGCCAGCCCGCTCCCCTCCCATGTGAC
CCCAGGCAACCATTTTAATTTTTTTACAGCCAAGCGTAGTGGAGA
ACTGGCTGCGCGCCGGTTGCCAGGGACGCGGCAGCCAGCTTGGCG
GGGCTGGGGTGAAAGAGGACGCTGCGTGGGGGAGGGCTCCGCGGC
TTCTGACTGGCTGGAGCATCAGGATTCATTTGCATAATATTTTCT
GACGGCTTCTGATTGGCTGAAGCCAGAGGGGCGGTGGTGGGGTGA
TTCC

In some embodiments, the intronic domain that promotes trans-splicing primarily in the brain comprises or consists of sequences from the gene MIDI IP1. In some embodiments, the sequences from gene MIDI IP1 comprise or consist of:

(SEQ ID NO: 206)
TGAGTTGTTGCGGGGTCTGGGGGAGCTGGGAGGCCCGGTTTGGGA
AGTTTTGCAGGCAGGTGCATGAGCCATCTTTCTCTGAAAGTGTTT
AAGAAATGTTCAAGAACAGCGGCCAGAGTCAAGAAACTGGGGGAT
GAAGGGTTATCTCGAGACTAGACGGAAGGAAACGTATCAGGGGAG
GGGTGGAGGCCAGAAGGAAGGAGTTAGGTAGGGGCCCTCTCCCAA
GCTGGCGTTTTTGCTGGGTGGAATCCTTTTGCTAGATCCCTTGGA
TTTCCGTTTTCCGTGTGTTATCACACTGTTGGAACTGGGAAGAAG
AGAAAAAGAGGCAAGATTTTGGCATTTAGTAAGTTAAGTCTGAAT
ACCGAGTTTTCATCCGGGTGCATACGTACTTAAAATACCAAATGG
GTCCAGGAAGGGGGACAAAGTACGCTTTAATTTATGAGAAGATCA
GCTACTAGAAACTGTATATCTGTTTTTCTCGAGTTTTCTTCTTGC
AGTG

In some embodiments, the intronic domain that promotes trans-splicing primarily in the brain comprises or consists of sequences from the gene SYN1. In some embodiments, the sequences from gene SYN1 comprise or consist of:

(SEQ ID NO: 207)
GTGAGTTTGGGGAAAATAGAGTGGGCAGGAGGGAAGAGTCATAAC
TACTGCAATAATATTAAATATTGGTATATTATCAGCAGTGCGATA
TCAAGAGTGTGGCTGCTGTGTGCACGTGGAGTCATGAAGTTGCTT
TGTGATAGGGTCTACCGTGAATTTCCTTATGCTTACAAGGAAAAC
TAAATCATGATCCATTCTAATATGTATCAGATCCTGTTACTCATG
TTGTCTATTTTTGCCAGTATTAAAAACCTATTTCTAATGTGAATT
GACAGAATTAATGTAATGAATCAGAATATATTACATATAGAAATT
AAGTCATTATTACTAACATTATCAAATATTCCTTCTGATAGAAAT
TTTACCTATATTCAAATTAGGATTGCCAATAACATTTATCAATAA
TAATGTTATAAATTAAATTGTTGGCAAGGCACGGTGGCTCATGCC
TGTAATCCCAGCACTTTGAGAGGCTGAGGTGAGCAGATCATGAGG
TCAG

In some embodiments, the intronic domain that promotes trans-splicing primarily in the brain comprises or consists of sequences from the gene SYN1. In some embodiments, the sequences from gene SYN1 comprise or consist of:

(SEQ ID NO: 208)
GATCACCAGGTGGGCAGAGGGTAGAAAGGGTGCACTAGGCAGAGG
GAACAGCTCACACATGTGTGCAGAGGTGAGAAACCACTTTGCAAT
TTCTGGGAGCTATGGAGATTTAAAAACTTGGTCTTTGTTTCGAGA
GTGATTGGGAGCTTCTGGGGGAACTCTCAACACTCCTACCCCTGC
CACACTAGGTCTCTGAAGCCGGATTTTGTGCTGATCCGCCAGCAC
GCCTTCAGCATGGCACGCAACGGAGACTACCGCAGTTTGGTCATT
GGGCTGCAGTATGCTGGAATCCCCAGTGTTAACTCCTTGCATTCT
GTCTACAACTTCTGTGACAAGCCCTGGGTGGTAAGTAACAGGAAG
GGTGCCACTGGCAGAGGGAACAGCTCATGCATGTATGTGGGAGCT
TGAAACCAGTCTGCGATTTGGGGAAGTGAGGGTGTTCAGAAACTT
ACAGATTCGTGGTGAAGGACTCACTGACCCAGCTCTCCTGGTTCT
CATA

In some embodiments, the intronic domain that promotes trans-splicing primarily in the brain comprises or consists of sequences from the gene PQBP1. In some embodiments, the sequences from gene PQBP1 comprise or consist of:

(SEQ ID NO: 209)
GCGTCTGGCCGCGCAGTCAGGCCACGCCCCCAGCGTAGACGCACA
CCGGCGTCGGAGCTTTCCGCCTATTACCGGGCAGCGATTGGGTCG
CTATACCGCATGTCACGCCAAGGACGCACTCTCATTGGCCAATCG
AGTTTCTGCCATTTGTTCATTGCCTCCTGAGCGTAGTCCAGTTAC
TTTCAGGCTCGGGGAGTGAAGGCCTCGTTGAGAGAAGGTCTCATT
CGGTGTTTTGGGAAGAGAGTCGTGTGGGCCCAGGTATCGTAGCGG
CGACACGAGAGAGACGGGCGGTGTGACAGCCTTCCACTACCTGCA
CGAGTGTATTGGTAACGTTGGGGTGGGTGCACTCTTTTGGAGCTG
GAGGAAGTGCTGCCCTCTGCTCCCCCATCCCAGCGCTTTGGTTTC
TCCCACCCGGCAGTCATGGGGACGCTGGATGAGCCCATTTCTGAA
GTGCGGAGGGGCCGGGCTTCTTAGGCTGTGGGCGGAGCCTGGGCC
TGGG

In some embodiments, the intronic domain that promotes trans-splicing primarily in the brain comprises or consists of sequences from the gene PQBP1. In some embodiments, the sequences from gene PQBP1 comprise or consist of:

(SEQ ID NO: 210)
TGCGTATGTTCTGTCCACCCAGCTACTGGTTCATTTGAGTAGATC
GGAGTCAGATGAGTACATGTTTACGGGAGGCGCTTGGTTACGGGA
TCCAGATAGGAATCTTTATCTGAGCTGCTTGTCAGTTTGTTCGTC
TGTCCCTAGGTCTGTCTGCTATCAGCTATGCCGCTGCCCGTTGCG
CTGCAGACCCGCTTGGCCAAGAGAGGCATCCTCAAACATCTGGAG
CCTGGTGAGACAGCTAAAAGCAGATGGTCCTAACACGTGCCAGCC
TCGACAGGCACTTTTGTTGTTGATACTGACGCTTGATACCTAGCA
TGGGCCAACTGTGCACCAAGCAATGGGGTGGCAGGCATTTTTCGT
TGTTAACAATGTGGATTCCTGACATCCATCCCTACCGCGTGTCAA
GTGGAGGGGGTTGGGGGAATAGATATTTCATTCTTTTTTTTTTTT
TTTTTTTTTTTTTTTTTTTTTGAGGCAGAGTCTTATTGTGCACCC
AGGC

In some embodiments, the intronic domain that promotes trans-splicing primarily in the brain comprises or consists of sequences from the gene CD99L2. In some embodiments, the sequences from gene CD99L2 comprise or consist of:

(SEQ ID NO: 211)
GTAACGCTGCTTTGCCTTCCTCACTTTCCTTTTGCTTCAGCCTTG
ATGCTTCCTCATTCGCCCCCTGCTCTGGCGCTTCCTTGCCGCTCT
TCTCCCTCCTGCTTCCCTGGCACTCACCGACGTGGCTGTAACCAG
GACCATCTTTAGCTTACCCTGATGCGTGTGAGAGGATGCCACCTG
CCAGACACTCTGTCCCCAGGCTGGATGTGGCAGGGGTGGGGGGAG
AGTTGAGGTGCCATGGTGAGGACTCAGGTGCCCACTGGTGGTCAT
GCCTGGGGAGCACATAGAGGGCCACCCACAGGAAGGAGACGATCC
AGCGCCCACTGGGTTGGCAGCTAGCAGTGATCACGAGGCTCAGGG
TGTGTCTCCTGGCCCCCACCTGTCAGCTGGGAAGGTGCACCTGGG
GCAGCTTCCACTGCACTCATCACCTGCCCCGCTCTGCGTGGGGCC
GGCCACACCTGCCAAAGAGCAACGCCCGCATTTGCCACAGGCCCA
TTTG

In some embodiments, the intronic domain that promotes trans-splicing primarily in the brain comprises or consists of sequences from the gene CD99L2. In some embodiments, the sequences from gene CD99L2 comprise or consist of:

(SEQ ID NO: 212)
CATGGGGCGGGATTGGGCCGGTTGGTTTCTGAGTGTGCGACTCCC
AGCTCGCCTTGCCCCCTCCGTGATCAGGAGTCTCTGGACCGTCCA
TTCCGTGGCGGGGCTGCGTGTACATCATGTCCCTGGCACCTGGCC
CGGGCCAGAATCAGGAGGAGTCCCGCAGACGTTTGTGGGGTGGCT
GAAAACACTGAGAAGCTGGGGGTTGGGCCTCCTGTCCCCACTCTT
TCCCCTGAGTCTCAGCCACTGCCACATGGAGATGGCGGTAGAGGG
GACCGGGGTGCAGAACACCAAGCTCTGGGGAGACAGCAGGAGGTG
GCTGCGAGGAAGGCCTGCCCCCACCACTGTGGCCCTCAGGTGCTC
TCGGCCTCTGCTGCTGCCAAAAGGCCCATGCTGGGGACCCTTGGC
TTCCCTTGCCTTTGCCCTCAGTGTGGAAAGCCCGCATGTTTGCTG
TCAGGAGCGCTCTTACTCCAGGGATTCTAAGCTTTGTGTCTTGCC
TTCA

In some embodiments, the intronic domain that promotes trans-splicing primarily in the brain comprises or consists of sequences from the gene L1CAM. In some embodiments, the sequences from gene L1CAM comprise or consist of:

(SEQ ID NO: 213)
GTAAGGCAGGGGCGGTGGGGAGGGGCCAACAGCAAGGTCTCCCTAT
AGACAATGTGCGCCTGGTGTGCCATTGTCCCCTGGCTGCCGGGGC
TCTGACAGGACTCTCACTTCCCAGCCCCCAGGCCCGGCAGCTCCT
CATTTCAGGGAGAGGCCAACACGCCCCAGCCCCTCCTCTCCACAC
AGATACCTTCACTGCTCCCCTCTCCCCAAGGGAGGCCCCAGACAG
CTTCCCAGACAGGAACCAGGCCCCATAATCCCCTGGCCGAGCGCA
CATTTTCTCCCTCACAGTCCCCAGGATCTGGGGAATGCCCAAAGA
TGACAGCTCCAGACCTGCCTGCCAGGGTGGCCAGAGCTGCTCTTT
CTGAGCGCATTCTAAACAAATGGAAGGCAGGCGGGCGTCTGCCTC
ACCCCCAGCCAGGCTCCTGCAGGGGCTCGGCAGTGCTCTCACTCG
CACCTGCCCTGTGCCTCTGCAGTGACAACGAGGAGAAGGCCTTTG
GCA

In some embodiments, the intronic domain that promotes trans-splicing primarily in the brain comprises or consists of sequences from the gene L1CAM. In some embodiments, the sequences from gene L1CAM comprise or consist of:

(SEQ ID NO: 214)
GCCTCTGAGCCCCACGTGACGGGGGTGGAGCCGAGGGCAGAGAGG
AGCACCTCGCCCCTTCCACCCTTCTGCAAGGCCTCCTGGATTCCC
CGAGCTCCCCACAAGATGACAGGCGCATGGTGGGGGCAGGCAGGG
GGATCATCCTCCCAGGCCGGGGTTGGGGCAACAGGCAAGTGGTGG
CTGGACCGCAGGGGGTGAGCGGTTTCTTTCAAGGCCTCTTGCCTC
TGGGCGCTGTTGAGACAGAGTGCTGGGGGTGCGGGGCGTAGAGGG
GCCTGGAGCCCCGGGTCAGGCTGGGGCGGGAGAAGAAGCTGTCCC
TCTCTGTGTCTCCAGTGAAGGATAAGGAGGACACCCAGGTGGACT
CTGAGGCCCGACCGATGAAAGATGAGACCTTCGGCGAGTACAGGT
GAGCCGGGGCAGGAGTGGGTGCTGGCACTCAGCTCCACCCTGGCC
TTCACATCTCACCCCCTCTCTCTCTTCTCTGTGCTGCCGGGTGAC
TTCA

In some embodiments, the intronic domain that promotes trans-splicing primarily in the brain comprises or consists of sequences from the gene CCDC3. In some embodiments, the sequences from gene CCDC3 comprise or consist of:

(SEQ ID NO: 215)
GTAAGTAAGCCATTTGATTTCTGCTGCCTTTGTTTTCCCATAAAA
GGGCGTTTAAAAAAATATAATTCTCCAAGAAATCTCTAGTTGTTT
TGAAATAAAAAGTTACTTTTCGCTCTTCATCTCATTTATTCCAGC
TATTCTCTGCTTTGCCCTCCTTGGTTTGTGGAGCCTCATACAAAT
TCATTAGTACTTTTCCAGCGTCACAGGTTGGAACTGAGGGTTCCA
AAGAGCAAAATGCATTTTGGCTGAAGCAGAGCCAGGAATCCATGT
TGTTTGCCATTGGCGCCATCCAATTCACTTGTTCTGAATAAGGGA
ATAAAGTTAATATTTATTCCCTTCATGATTCTTGTATTTCAAGAA
AAAAAAGTGCTCTACAGGCTTTTTCCCCCCAGCAGGTACTGATTG
ATTTGTTTGAATTTTGAGCTGCAATCAAGGATGGAGAATCTAACT
GATCTCCTTATTCACCAAATATTTATGATTTGTGCATAGAATATT
GACT

In some embodiments, the intronic domain that promotes trans-splicing primarily in the brain comprises or consists of sequences from the gene CCDC3. In some embodiments, the sequences from gene CCDC3 comprise or consist of:

(SEQ ID NO: 216)
TGATGACTATTACCAGTTTTCTCTAAGCCTAGGCCCAGCCCTGGC
CCATGGCCTCAGCCCTTGGATTGATATGGCTTATTTAGGTATTTA
AAGGTCGACACCTGCCTCCTGCTGTTTAATTCCCTGCAGTCTGAC
AGATTCACCAATTACCAAGGAGAAAGCCTGCCTTCCTTGTGCTGT
GTGTAAATGAGGCTTCCATCAGCACAGACACACACACAGGCTGCA
AATGGACCAGCCCTGCCCCCAATTCCACACCCACGGCCACAGCAA
AGAATGCTGTGTTCCCCTTCCCCAGCCCTCACACCCAGGTTAATA
CCTGGGCTCACAGCCTCAGCCCCACACCCAGGTTAATACCTGGGC
TAATTTCATTTTTTCACTTTTTGGGTTTTCAGATGTACACCCCAG
CGGAATCAAAATGACAGATTTATGTCACATTGTGGGTTTGGATCA
GAAAGCACCCTTGAGCAACACTTTGATTCTGTGTTTCTTCCCGTC
CTCA

In some embodiments, the intronic domain that promotes trans-splicing primarily in the brain comprises or consists of sequences from the gene FAM107B. In some embodiments, the sequences from gene FAM107B comprise or consist of:

(SEQ ID NO: 217)
GTAAGAATAAACACTGGGACTTTTCTATGGCTGTTGAATAATGTA
GTCCTTCTCTAGAAAATGTTGTGTGAACTTAGACTTGTTAACACA
ATAGCAGACTTGTATGAGACTAAATAAAAATTATATGTTGTTATA
GTGAGTGGTATTGAATAGTGTGTCTTTGCCAGGAAGAGAGCTTTG
TAATAGGCTGTGTCTTGTGTAGTTGTGAACTATTTTTTTTTCCTA
AAATTTCTTCAGATGCCAACCATGTCTGTCAAACTCTGGATGAGT
CGCCTTTTACTTAGCTTTTTAAGAACTCTTTTTGGATTCACTAAC
TTTTATCTGCATTATTTTATCCATGCTCTATTAAGTGTCTAGGAT
CCTGATAATTATGCATTTTCTCTGAAGATATCTATTTCCAGTAAA
ATTTGACTATCTAGTGAAGATTGTAGGCCTGATAATATATTTTGA
AGTACTTGTTATGATCTGAATGTGTGTGTCACCCCCAAAAATGCA
TATG

In some embodiments, the intronic domain that promotes trans-splicing primarily in the brain comprises or consists of sequences from the gene FAM107B. In some embodiments, the sequences from gene FAM107B comprise or consist of:

(SEQ ID NO: 218)
CGAGAACTTGTCCTCCTCCAATTGGAGGTTGACTCACGCATGCTT
CCTGCAGTCAGGTTGTGTGAGTGTTGCAACCTGGTACCCACCCAG
GGCTGGAGAGTCTGCGTGCATATTTGCTTACAAGTAGAGGAGCTG
ATTTTTGTACAGACAAGTACATTTCACCAGTGCTGCCAGCACGTT
CTCTTGGTCTGGAAGGTCAGTAACTGGTACCAGAGTTGGTACGGT
TTGTTTTTCATTGTTTGCTCTGGCTTCTGTGCCGGATGAGGTCAT
TCAGTTGCATACAGTTCCTGCTTTCAGAAGTTGCTAGAGTGTTTC
TCTTTGAACTTGAACTGTGGCTCTTTTGATATCCAGAAAGAGAGT
TCTTTGTGGGGATGGTTAATTCTTGTTTTGGGTGCTTTTCTTCAC
CGCTCTAAGGAACTACCACAGTCAGAAGGTGCATTCTTACTGCGA
TCTTTTTATTTGCTACCTTGGGCTTAAGGAGCCTGCCTATATCTA
CTGA

In some embodiments, the intronic domain that promotes trans-splicing primarily in the brain comprises or consists of sequences from the gene NEBL. In some embodiments, the sequences from gene NEBL comprise or consist of:

(SEQ ID NO: 219)
GTAAGCTGACGGCGTTTGTCGGCCGGTGTGTGTGGTCGAAAAGAT
CATGCTTGCTCTAGCTTCAGAATTACCCTCTTGAGTCATCTGACT
CCTCATCACATGTTGGTTGTGGTACCCAATTTAAAATGGACCGCA
GGTAAAGTATTATTTTTGTACGATGTGGAATGGGATTTCTGGCAT
TCACCTGGTATCATCTCAAGTCTTTTCTATTATTGGAAGCTCTGT
CTTTAATACCTTGCTAATTTTAAGAATGTTCCTCCTTGTATCACA
CACCAGGGCCTGTTGTGAGGTGGGGGGAGGGGGGAGGGATAGCAT
TAGGAGATACACCTAATGTAAATGACGAGTTAAGGGTGCAGCACA
CCAACGTGGCACATGTATACGTATGTAACAAACCTGCATGTTGTG
CACATGTACCCTAGAACTTCAAGTATAATAATAATAAAAAAAAGA
ATGTTCCTCCTTGTTAAGCAAAGAAGCAGCAGAGATATGAGTGAT
CACA

In some embodiments, the intronic domain that promotes trans-splicing primarily in the brain comprises or consists of sequences from the gene NEBL. In some embodiments, the sequences from gene NEBL comprise or consist of:

(SEQ ID NO: 220)
AAAAAAAGTATATTTTGAAGTATAAGTTTTTGTAATTATATTAAA
AGTGGTATATATTGTTACCTATAAAATTTCAAACTGATTTTAAAT
AATGCCATTTTCATGAATGATTGCCTGGAAATAGATATCACTTTA
TTGCATACTGAAGTTGAAGAGATAGGAAATTTAATTTATATTTAA
ATTAGTATATTTTAGGAAGTAACCAGGATTTGAGAATATGTATTT
TTACAAGCTCTCATTAATAATATATATATAGTTTTATATATAATT
TAAAATAGATATGAGCTATAATGATTAAAAACTTTGTGAAAATAT
CAGCACTTGATTTCTGATGCTAAGAAACTGAAGTTCCAGACTTTA
GTTTTGGAGTATTTGGAAAAGATACATACATGTAGACAATCTGCA
CGCAGCCAGTGCAGTGTACACCTGTCATTTAATAAAAATGTTGTG
AAAGAGGTAACTATCCGCAAAATTCAGTATGATGATTTTTCCTTT
TTCA

In some embodiments, the intronic domain that promotes trans-splicing primarily in the brain comprises or consists of sequences from the gene HNRNPF. In some embodiments, the sequences from gene HNRNPF comprise or consist of:

(SEQ ID NO: 221)
GTGAGGGGCGGCCGAAGTCTCCGAGGTCGAAGGCCGCACACCCGG
GACCTAGCGGGGACCGGGTCCCCCTGGGGGTGGGGACGGGGGGTG
GGATTTGAGATTAGGGAGACTATCCGAAGTCCAGGGCCCCGTACC
CGGACTACCTGGGGACTGGGCCTATTGGGGTGGGGATCGTTGGGC
TGGGGCTGGGATTTGGGATCAGGGAAGAGCGATCTGAGTTTAAGC
GCCGCAAACCTGGGACCGAGCGAGGACTGCACATAATGAGGGGCC
GTGGCGCTGAGATTTGGGTGACGGGATTCGAGGTCGAGGGGCCCG
TACGTGGGACCGGCAAGAACTGGACCCACTGGGATCGGGAGACGC
AGCGTCGGAGGTCGAGAGCCGCACGCCCAGACAGTGGTCCCACGC
GGTGAGCACCGCGGGCTGAGATTTTGGAGTCAGGAAAGCGCGGGG
AAGGCACGGACGGCCGGGTGCGTTAGTCCGGCCCCCTGGCGGGGA
GCTG

In some embodiments, the intronic domain that promotes trans-splicing primarily in the brain comprises or consists of sequences from the gene HNRNPF. In some embodiments, the sequences from gene HNRNPF comprise or consist of:

(SEQ ID NO: 222)
CCCTATCCCCTCCCCTCCCCTCCCTTCTCCTCTCCAGAAACGTCC
TCGCTTGTCCGCCTCCACCGCCTCCCCCGCCAGCCGTGTCGGCTC
TGCTCCGCGCCCAGCCGGCCAACCGGCGGCTCAGCTCTGGCGCGT
CACAATGCGCCGTCGCGGCCCCGCCCCCGGACGCCGGACGCAGAA
ACTCCGCCCTGCCGCGGTCCTTGCGTCCTTCCGGCTCCGTCGTGG
AAGCAGGACTGCGCCGCGTCTTCCTCAAGGTGGGGTTCGGGGGGC
CACCGAATCGCTGTCACGCCGGCAGGTAGCAGGGGGTGGGGACGC
GGGCTCCTTGGGCTTCTGTTTCCTGGTGTCCAGGGCAAAGGGCTG
AGCCGGGCAGTAGGGCCTAGTCGGGGCCTGGCTCGCCGGGAGCAG
GAGGGCTTCCGGCAAAGCCGCGTGCGCTCGACGGGCAGGCGCTGA
GGATTGGGGCCGGCAGCGCGCCTGACCCCGCCCCTCGTAGGCTCC
GCCC

In some embodiments, the intronic domain that promotes trans-splicing primarily in the brain comprises or consists of sequences from the gene FAM213A. In some embodiments, the sequences from gene FAM213A comprise or consist of:

(SEQ ID NO: 223)
AGGGAGTGTGGGTTGAGGGAAGCCACCGATGTGGCAGAAAAGGAA
GGGAAAGCAAAAGGAGGTAAAGAATAGACAGCAGATGAGGGGCCT
CAGAGGTGCCAGCTGACTTTCCAGGCCTGGGGTGAGGAATGGGGC
AAGGGAGCAGGCTGCACGAGGAGCCACAGAGAAAAGGACATCTTC
TTTTATTTGCGCTAGTATGACATGTGGGCTGTGTTACGAGCTTTG
CTTCTTTAGTCTGCACCGGATAGCAGAGGAGTCTGGTGGCACTTG
GGAAATGTTATAATCTGCATGACTGCAAATGACTCAAAGCTTTGC
TCAACTTTTTGAAGGGCTAGGGGGCCTTGGCAGCTTTCCATTTAG
ATGTTTTCTGGAGACTGACTGTTGGCAGCTGAAGGAGCCCCAGGG
TTTCTGGGTGGCCTGGTCCTGCCTATGGAGTGACTGAGCCTAGGG
ACCTAGTCCCGCCTCCAGCCCTCCCCTTGCCGCCTGCCCAGGCCC
GTCT

In some embodiments, the intronic domain that promotes trans-splicing primarily in the brain comprises or consists of sequences from the gene FAM213A. In some embodiments, the sequences from gene FAM213A comprise or consist of:

(SEQ ID NO: 224)
TGAGTGCCTTGTTCCCAAGTTTGAAGCGTCTGAGGTTGGGGGTAA
GAGACCGTTTGAGGAAGGGAGTTGCCTTGTATAGGAAAGAGACAG
GGTCTCGAAGCTGGAAACTGAATTTGGGGCGGTGGGGGCAGTGTT
GAGGAGCCTAGAATGTTCTGACTCCAAAGTGACCTGTTTTTGTTA
GACTCATAGGATGTCACTGGGGATCATTAGGAACTTCCTCATGGA
GTTGAGAAAAATGGGGCCTGGGGAGGAATTGATTCAGCTTAGCCT
AGGGAGCCCGACTGGGTCTCGTCCTCTTGCACCCATGTTCAGACC
AGAGCTGCCACTCTGAGCTGCCACAGCTGTTGTTTGTTTGTGGCT
TTATCCTACTTCACCTTCTGTGGGAGAGGGTGGATCAGATAAGCC
CAGCAGTGGGGAACTGAATGCTATAGCTGTGTCAGGTGGGGGCTG
GCTCTTCTGTAAACAACCCTACTATTCTGTTTCAGAGAAACTGAC
CCTT

In some embodiments, the intronic domain that promotes trans-splicing primarily in the brain comprises or consists of sequences from the gene NDUFB8. In some embodiments, the sequences from gene NDUFB8 comprise or consist of:

(SEQ ID NO: 225)
GTGAAGATGGCGGTGGCCAGGGCCGGGGTCTTGGGAGTCCAGTGG
CTGCAAAGGGCATCCCGGAACGTGATGCCGCTGGGCGCACGGACA
GGTCCGCGAGAACGGGCGAGACCTCAGTGTGGGAAGGGGGTACCT
GAAATTTGGTGTATCGGGAGGGGGGATCGTAGATGCCGCGGCGGC
AGCCTCCGGGTAGGGAGTGGAGGTGGCGTTGATATATCCAGAAAA
TGGGGTGGAGGATCGCACTGATCCTGCGAAGTCCCGGGCTCGGAC
CTCTGCAGCCCCTGCGACTTGAAGCCTTCGGAGCGGCTGGAGAGC
GGGCTCGCTTCTGACCTGTTCTCTTCTGCGTTCTAGCCTCCCACA
TGACCAAGGACATGTTCCCGGGGCCCTATCCTAGGACCCCAGAAG
AACGGGCCGCCGCCGCCAAGAAGTATAATATGCGTGTGGAAGACT
ACGAACCTTACCCGGATGATGGCATGGGGTGAGACAGAGAGGGTA
TGCT

In some embodiments, the intronic domain that promotes trans-splicing primarily in the brain comprises or consists of sequences from the gene NDUFB8. In some embodiments, the sequences from gene NDUFB8 comprise or consist of:

(SEQ ID NO: 226)
TGGGTTGAGGTGACTGAGACATGCAGATGGAAACACGGACTGAAA
CTAAGGAGCCAATTCAGGGCCGAATTGTGTGTTGCACTGGCGTCT
AAGTGAAGATCAACCCATTTGGAGAACAGAACAACAAGCAAGACC
AACAGGTGAATTTGAAGAATGATCATGTTTGGGGATGGAAGGAAG
TGCCCGTGAAGGAGAGGAAAGCAAGCAGCCAGAGAAGAGAGTAAG
ACAGGCTCATTTCACTAGTACAGCTCCTGGGGGAGGTGGTTAAGC
TGTCTACCTGAGTTGCTCAGGTTCCGAGTTCCCATCATCCCTGGA
TGGGTCTAAGTCAGCTCAGGGAGAAAAAGGACACAACATTCTGAA
ATGTCAGAGACCTGCCTGGTTTTCCCTTCTTCCCGCTTATCCTTC
CCAGCCTCTATCCAACCCTTGGCTTCCCAACTGCGTATTTAAACC
CCGCCCCTGTTGAACCCTTCGGCTTTTCTTCACTCCCGGCTTCCT
CCGT

In some embodiments, the intronic domain that promotes trans-splicing primarily in the brain comprises or consists of sequences from the gene PSD. In some embodiments, the sequences from gene PSD comprise or consist of:

(SEQ ID NO: 227)
GTGAGTGCCCTCCTCCTGCCCATCCCGGACCCCTTCCCCCACCTC
CCATCCTGCTCCCCTCCCTGTCCCTGGATTTTTTCATTCCTTCTC
AGGCCACACTTGAGCCAGGACTGGCATCCTGGGACAGAAACCCTG
GGCCCAGGTCCCAAAGGGCTGTGCTTTCTCCCCAGGAGCCTGGAG
CAGATGCAGTCCTGGATCACTCGCATCAATGTAGTAGCCGCTATG
TTCTCTGCGCCCCCCTTCCCAGCTGCTGTTAGCTCCCAAAAGAAG
TTCAGCCGCCCTCTCCTGCCCAGCGCTGCCACCCGCCTCTCCCAG
GTAGCCCAAGCTCTGTCCACCTAGTGCCAGTGGGATGGAGGGTGT
TAGGCTCAGGCCCACTGCACTCAAGGCTGCTGCGTGTGATCGTGG
AGATGTCCCCTGCACCAGGATGCTGGCCACAGGGGTGAGCAGGGA
CTGAAATCCAGCCCCCACTGGGTCCCGCTTGCCGGCTGGGAACCC
AAAC

In some embodiments, the intronic domain that promotes trans-splicing primarily in the brain comprises or consists of sequences from the gene PSD. In some embodiments, the sequences from gene PSD comprise or consist of:

(SEQ ID NO: 228)
GAGGTGATCACACCTGCAGAGACGAGGAGGAGCTGAGACGCTCTC
TGTCTGAGTTGGCCGACCCCAACCCCAAGGTCATCAAGCGGATCA
GCGGGGGCAGTGGCAGTGGCTCCAGCCCTTTCCTGGACCTGACTC
CCGAGCCTGGGGCTGCCGTCTACAAGCACGGGGCCCTGGTGCGAA
AGGTGCACGCAGACCCTGACTGCAGGAAGAGTATGTGGCCGAGTT
GCGGGGGGCGGGCGTCTAGGGGATGGATGGGAGTGGGCCTGTTCC
AGGGCCCCCTGACCTGTCTTCTCTCCCCGCAGCACCTCGGGGCAA
GCGGGGCTGGAAGAGCTTCCACGGGATCCTCAAGGGCATGATCCT
CTACCTGCAGAAGGTGCGGGGTCAGGCCAGGGGGCTGGGGCTGGG
ACTGAGTTGGCTGGTGGGTGTGGGGTGATGGGTGGGAATAGGAGG
GGCTGGGCCAGGCTGCATTTGGGGCCAGGACAAGATGGACACCCC
TGCA

In some embodiments, the intronic domain that promotes trans-splicing primarily in the brain comprises or consists of sequences from the gene GSTO1. In some embodiments, the sequences from gene GSTO1 comprise or consist of:

(SEQ ID NO: 229)
AGAGGCCCTCCCAGTAGTCACGTTAAGCTAGTACTGACAGAAAAA
GGAAAAGCCTGTCCAGGTGGCTGGGCAACCCTGCAGCGAGGCATA
GATGAGCTTGTAAATCAAAGGAAATGACCAAGGTGAGTCTCAATC
CTTTTAGAGGTTTATTTTGCCAAGGTTGAGGACACATCTGAGAAA
AAGGAACACAAAATCACAGGAACATCTGTGATCTGTGCTCTTTCC
AAAGAGGGCTTGAGGACTTCAATATTTAAAGGGAAAGAGCAGGCA
GTAGGGGAAAGTGGAAAGTAAAAAGGGAAGGTAGATAAAAGGGGA
GAGTAGTTGCATTCTTTGGGGCCTTTGATCAGCATTCACTGAATG
CGCATGTTACATTCGTGAAAGGAGGTCGTAGAGGAATAGTCAGTT
ATGCATTCGTCTAGTGCTTGGTGAATCTGCATTTTTACAAGATTA
AATATAGGGTAGAGGAAGCGGTCAAATTGCATTTTTCTCCAGCGA
GAGG

In some embodiments, the intronic domain that promotes trans-splicing primarily in the brain comprises or consists of sequences from the gene GSTO1. In some embodiments, the sequences from gene GSTO1 comprise or consist of:

(SEQ ID NO: 230)
TGAGTGAGCCATGAGGTCGGGCCACAGGCTCTGAAGTCGTGAAGC
CTTGGGACGGAGCGGGTGGTGCTTCGAGGTCAGTGTCACGGGAGG
GAGGTCAGGGTCAGGGTCAGACGTGGAGCCCCGTGGAGTGCGGAG
TGGTGACCCCTTCGTTCGGGGGCGGAGGGATGACTGAGCATTTAT
AACTTTTGTGTATCTCCCCGTGGGTGCAGCCCTTGGCCAGCGAGA
TGCTTTGACACAGCCCCTTAAGATGTTTTAAGGGCTATCCCATAA
AGCCGGGAAGGCACAACGGGTGGAGCTGGCGGCCGCCGGGGGCAG
GCACTTTTGAGCTAAGGAGGAAGCGGGGGAGGGAAGGAGGGCGGG
AAGGACGCGCCACCTACTTCCTGAATCCCCTGCAAACCCCAGAGG
AGCTCGGCCTGCGCTGCGCCACGATGTCCGGGGAGTCAGCCAGGA
GCTTGGGGAAGGGTGAGGCCTGCCCGCCGCGAAGAGGGGGTGATC
TCGG

In some embodiments, the intronic domain that promotes trans-splicing primarily in the brain comprises or consists of sequences from the gene TACC2. In some embodiments, the sequences from gene TACC2 comprise or consist of:

(SEQ ID NO: 231)
CGTATGCCTTCCTGCAGGATTTCTCTGTCTTCCTGTCCCACTCTT
GACCCTTGCTTTAGCCACACTGGCCTCCTCGCTGTTTCTGTAACG
CACCAGGTGCTCCTGCCGCAGAGCCTTTACTCGCACTATTCCTTC
TACCCAGGATGCTTCCTCCCCAGGAGTCCTCCTGTTTCATGCACT
TACCTTGATTCTGGCCTTTGCTTAGATGTCTCCTCAGAAGATCTT
GCCTGACTACCTTATTTTAAAAGTGAAACTCTCCTATCCCGGCTT
TATTTTTCTCCTTAGCATGTATCACCCTCTGAAATTCTGTACATT
GTTTGTTTGTTTTTGAGACAGAGTCTCACTCACTCTGTCACCCAG
GCTAGAGTACAGTGGCGCAATCTCGGCTCTCTGCAACCTCTGGCT
CCCAGGCTCAAGCGATTCTCCTGCCTCAGGCTCCGGAATAGCTGG
GATTACAGGCGCTCACCACCATGCCTGGCTAATTTTTGTGTTTTT
TTAG

In some embodiments, the intronic domain that promotes trans-splicing primarily in the brain comprises or consists of sequences from the gene TACC2. In some embodiments, the sequences from gene TACC2 comprise or consist of:

(SEQ ID NO: 232)
TGTGAGACACCATGCCCGGCCCTGGTGGTTTATTCGCTATCTTCC
CCTGAAGAATGTGACTTCCAGGAAGGGAAAGGTTTTGTCTATTTC
TGTTCCTTGTTGAATCTCTAGTACCTAGAACCATGTCTGGTCCTG
ATTAGGCACTTGGTGAATGTTTTTGAATGAATACTCTGAGCTGTT
CTTTAATCGGTCCTCATGATCCTCATTGTACAGATGAGGGAACTC
TGGGCGAACCTGGCCTGGGTCTTGAGCTAATTACGGGTAGGTCAG
GTTCTGTACCCAAGTAGTACACACAGTGATGGGCGGGGTGGCCTG
GGGCCGTGGTTTGTCAACCCCTACGCTGGAGGGTGATGTTTTGGT
ACAAGAGGAGAGGTGCCCCAATGCGTCCTGTGTCTGTAATTGATG
GCGTTGTCTGTGTTTCCCCAGGATGTGGTTCTGAGACAGTCCCTG
TCCCTGATGGCCCACGGAGCGACTCGGTGGAAGGAAGTCCCTTCC
GTCC

In some embodiments, the intronic domain that promotes trans-splicing primarily in the brain comprises or consists of sequences from the gene TM7SF2. In some embodiments, the sequences from gene TM7SF2 comprise or consist of:

(SEQ ID NO: 233)
TGCCTAGGGGACGGGCCCTCGCGCTGGAGTTAAGCGGCCGGGGGT
GGAGCTCCAGGCCTAGCGGGGAGGTCCACGGAGATTGGCCCCCAC
CCCAGGCTCCTTTGTGTGCCTCTGTTACGCCCAGGACAGACGCCG
GGGGCTCTCCCTATGCCCCTCGTCCCCACAGCCTTACCCCATTTA
CTGCTACTCCGTGCACCACAGCAGTGGCTGGGGTGATCCTCTGAA
GGCCAGGGTGGCAGCCTAATGCTTGGGGCAGGGTCTGTGGCCCCC
CTGAGTTTTGGGCTCTGCGTGTGTTTGCGGGGTGGGGATGTGGAG
GCGCGCAGCCCCAGGGCTCACTGTGCGCTGTGGTTCAGGCTTCCA
GGCCCTGGTGCTGACAGCCCTGTTGGTGGGGCTGGGGATGTCAGC
GGGGCTGCCTCTGGGGGCGCTCCCGGAAATGCTCCTGCCCTTGGC
GTTTGTCGCCACCCTCACCGCTTTCATCTTCAGCCTCTTTCTCTA
CATG

In some embodiments, the intronic domain that promotes trans-splicing primarily in the brain comprises or consists of sequences from the gene APBB1. In some embodiments, the sequences from gene APBB1 comprise or consist of:

(SEQ ID NO: 234)
GAATAGGGCTTGGGATTCGAGGGTGGGAGTTCATCCAGCATCAGA
GTCCTCCTCTCTAACTCTGTTATTTGCCTCTCTTGTGCTGCTGGA
CCCAGAGAGAGGTACTATGCCTATTTTCCCCCTCCTACACCTGGA
CCAGATGCCTGGAAGCACCCAAGCTCCCCTCTTCTGCCCTGCCCC
TTCCCACCCGCTATGCTCCACCATTACCCTCCACCTCAGCATGCT
TGCCTGTCTACCCCTCCCCCCCAAGAAGGCAGCACCTATGGGGGA
CCAGCTGTGGGCAGAAGCCCTGGAGGAGCTGTGATGGGCAGGGAG
GACGTGGGTGGGGTGGGGACCAGGCTCAGCACTGGATGGTACTGG
GAGACACTGAGGTGCCCCTTCCCCAACAGGGACTTTGCCTACGTA
GCTCGTGATAAGCTGACCCAGATGCTCAAGTGCCACGTGTTTCGC
TGTGAGGCACCTGCCAAGAACATCGCCACCAGCCTGCATGAGATC
TGCT

In some embodiments, the intronic domain that promotes trans-splicing primarily in the brain comprises or consists of sequences from the gene APBB1. In some embodiments, the sequences from gene APBB1 comprise or consist of:

(SEQ ID NO: 235)
GGGGAAGTAAGGACCTGAGCCGGGGGTAGGGGGTGGTACTGTGAG
AGTGTGAAGGGAATTATGAGAGATGGGAGGTCTGACCTGCCCACA
AGCAGGCTGGGGAAGGGGGATATTGGCCCATCCTAAGTGGCAGCC
CTGTGGGATGGATGAGCGACCCCAGGCCAGGACCTTGGGAGATAG
TCGCCTAACAACGGTGGAGTCAATGTGAGAGATGACACGTGACCT
CCACTAGAGTGAGGCCTGCAGGGCGGGCGGTCTGACACCAAAGCA
GGGGCCCCAGAGCTGTGGCTAACATCCCCTGAGCTGCAGGGGTCA
GAGATCAGCCACCATCCTGGCTGGAGTCCCACTGAGTGCCTGCTT
CTGGTCCTGCAGGGAAAGGATCTGCTACTGCAGCTGGAGGATGAG
ACACTAAAGCTAGTGGAGCCACAGAGCCAGGCACTGCTGCACGCC
CAACCCATCATCAGCATCCGCGTGTGGGGCGTCGGGCGGGACAGT
GGAA

In some embodiments, the intronic domain that promotes trans-splicing primarily in the brain comprises or consists of sequences from the gene EIF4G2. In some embodiments, the sequences from gene EIF4G2 comprise or consist of:

(SEQ ID NO: 236)
GTGGCTAAACAAAATGTAATTGTCAGTGTTTTGAGTACATTTTAG
GTTAGTCTTACAGATCTGCAATGCAGTGACAGTGCCAGGATCTGA
AATACGTTGTAATTGAATGAAGGCTTATAGATCTGTATGTTAGCA
GTGTCTAATTTTGGTGCTCTTTCTCGTTCCAGGTATTTCATTTCT
CCTGTCCCCTCCCCTCCCCACCCCATCTATTAATATTATTCTTTT
GAAGATTCTTCGTTGTCAAGCCGCCAAAGTGGAGAGTGCGATTGC
AGAAGGGGGTGCTTCTCGTTTCAGGTACTTGTAGATTTATTTTGG
GGAATCCTTTTGAAAAGGCCAGTCTCAAAAAGTAGTTAACACAGC
AAATAAGTCCGACTTAATGGGCTGCAATTTTCATTTGAAAAGTTC
TTAAGTTTTCATTGACAGCCTTCTAAGAACTGCCATCTGAAGAAT
AAATAAAATCAATTATTTGCCTTTCCTTTGGGTCCGAGTTCAGTC
TTCA

In some embodiments, the intronic domain that promotes trans-splicing primarily in the brain comprises or consists of sequences from the gene EIF4G2. In some embodiments, the sequences from gene EIF4G2 comprise or consist of:

(SEQ ID NO: 237)
AGTGTTGGCCAGGCCGCCGTGCGGTCATGGCCGGATGGCCGACGC
CTGCAGGGACGAGGCCGTGGCGGACCGAGGCACGGCTGTGCTCGG
ACGGCGAGGGAGCGTGGCTCGGACCGCTAGCGCCGTGGCGGGAGG
CACGCGGGCCGGCAGGGAAGGCCAGGCCGTTGGCGCGGGACCAGG
GAACCGCAGGCGTCCCGGAGCCTGGCCTCCCGGCGGGGAGGTGTG
TAGGACCTGGAGTCGGCAGGGGCTGCCCTTCGTCGGGGAGGAAAG
AGAGAGTGATGATTTCTGCCCCTTTTGTCAGCAGAGGAGGCCGTG
GCGAGGCGGTTGTCAGTCCCGGGGGGATTTATGGTGCAGGAGGGG
GCCCGCTTGGAGGCGGCGGGTGGGCAGGAGGCCGCCTTCCTGCTT
GGAGAACAAAGAGCTGGGCGCCGGGCTGGCGGGGGTGGCCTCAGC
TGCTCCTCCCCGTCGAGCGACCCGGGCCTCGCGGGGAGGTTGTCG
GAAG

In some embodiments, the intronic domain that promotes trans-splicing primarily in the brain comprises or consists of sequences from the gene SYT13. In some embodiments, the sequences from gene SYT13 comprise or consist of:

(SEQ ID NO: 238)
GTAGGGGTCTCCTGCCCCCTGCACTTCCATGGGCAGCACTCCAGT
GGGGCTTGTCAATGTCTCTGTAGATGCTGTGTGTGGCCTGATGAG
GAGGCCGAGTGCCCCGGCATTTGACGCTGGCCAAGCAGATGGGCA
CTGGGCCTCAGAATTATGGTCCTTTCTTCCTGACAACATGTTGCC
ATACCACTTCTCCCCCAGACCGCCAACTTTCCACTGGAAAATGGA
GTCCCTTTCTAGTCATATGTGTGCAACTATGAGAGAGGATAAAGA
GCCATGGTTAAAACCATGGGCTTCAGAGTCAAGGGTTTAATTTGC
TGCCCTTTCATACACTTATTGTGTGGCATGGGTCATGCTGTAACT
CCATGAACCTCGGTTTCTTCATCTATAAAATGAGACTAGACTATT
ATCGAGCCTGGCACAAAGGAGGTAACAAAAAACAT

In some embodiments, the intronic domain that promotes trans-splicing primarily in the brain comprises or consists of sequences from the gene SYT13. In some embodiments, the sequences from gene SYT13 comprise or consist of:

(SEQ ID NO: 239)
CAGGGAGCTCAGTGACTGGAGATGAGCAAAGGAAGGATTGGGGGC
GATATCAGAGAGATGAGCAGGATCAAGCTAGGCAAGCTGCAAGGA
GTCTGGATTTTGTGTGGTGGGAGCCACTGGCATTTCTGAGCCATT
AGGTGGAGAAGAGGAGGGAGGAAGGGAGAAGGGAGAAGGAAGAGG
CAAGGGGAGCAGATGAGAGCAGCAGAGTGGGGACCACCTGCAGCA
GGTCCAGGCAATATCCATGGGCGCAGCCATAGGATGGATGGGCCG
ATGGATGGAGGAGGTATGGATGAACAAGGCCCACATCTCAGTGGA
TTGTGGATTTGAGTGGGTTTGGAGATGATGTTAGACCTTCCTAGG
TTGTGCTCTTCAGAGCAGAATGAAACCACAACTCTCCCTCTGCAT
CCTACAACAGGTCAGTTCACCGTTTCTCCCTTCCATGGGGGGAAA
CAGACAAGAGGGCTCCTTTGTGGACTAATCAGAGTAATTTGTGTT
TGCA

In some embodiments, the intronic domain that promotes trans-splicing primarily in the brain comprises or consists of sequences from the gene MAPK8IP1. In some embodiments, the sequences from gene MAPK8IP1 comprise or consist of:

(SEQ ID NO: 240)
CTGCTAGGTGAACGTGTACTCCAGATCTCAGCCAGCCAGGTGGGG
GGCTGAGTGGAAGTGATTTTAGGTCCTTTTCACGATCAAGCAAAG
TGAGGCCCGGCCAAGTTGGGCAGCCAGGGATTGTGGCAGCCTCTG
TGGGCTCTGCCATTCCCCGTGCCTCACCCACCCTCCTTCACTTGG
CTGCTCAGCTCCCTCCTGCTCTTTCTGCCCCTCCTCAATTCACGC
TTGCTTTCCAGCCCTGGCCAAAAACAGTGACTGGGTGGACCAGTT
CCGGGTGAAGTTCCTGGGCTCAGTCCAGGTTCCCTATCACAAGGG
CAATGACGTCCTCTGTGCTGCTATGCAAAAGGTACCTGAGCCCTC
TCCCTTCTCCTCCCTTGGATGGGTCCCTGGGGCAGAGGGACGCAG
GTGTCTAGAGGCAGTAAAGGGCTCAGGCCCTGGGACAGGAGGGAT
CAGCAGAGGAACAGACAGCAGCTGACGTGGCTCCATTTGTCACCT
GTAG

In some embodiments, the intronic domain that promotes trans-splicing primarily in the brain comprises or consists of sequences from the gene MAPK8IP1. In some embodiments, the sequences from gene MAPK8IP1 comprise or consist of:

(SEQ ID NO: 241)
TGACTTCTTCCAACCCAGCCCCTTCCTTCCATGGCCCCAAGCTCT
CCCCCAAGACTTGTGATGAAGAGGCCATCTCCTGTCACCCTCACT
GCAGGCCAGGTGACCGCCCTCTTGCTTCTTTTCTCCCTCCTGTAG
GGGAATAAATGTAGCCACTTTTTCCAGTTAAAAAACATCTCTTTC
TGCGGATATCATCCAAAGAACAACAAGTAAGTGGGGGTGGGATGG
CAGTGGAGGAGGCACGGGTGGTCTGCAGCCTTGAGGTGGGTGGGT
GTGGGCCGAGCCCCCGTCCCAGCACAGACAGACCTGTCCCTGCAG
GTACTTTGGGTTCATCACCAAGCACCCCGCCGACCACCGGTTTGC
CTGCCACGTCTTTGTGTCTGAAGACTCCACCAAAGCCCTGGCAGA
GTCCGTGGGGTACGTGTACACCCTGCTGAGCACCCAGCCCGAGGG
AGCACGCCCAATCCCAGGCCCTGGGGCTGCTTCCTGGCGCTCAGC
TTTG

In some embodiments, the intronic domain that promotes trans-splicing primarily in the brain comprises or consists of sequences from the gene UBE2L6. In some embodiments, the sequences from gene UBE2L6 comprise or consist of:

(SEQ ID NO: 242)
GTGAGTCCCGGGTCCGAGCCTTCCGCGCGGGGATCGGGCGGTTGT
CCGGCGGGTCGTCCTTCGACGGCCTCCTGGCCTCGCCGGAGCGGG
TGCGGGGAGGCCCACGCAGCGACGGCTGGAGGAGGGGTGGGCGGG
GCTGCCGGGGGTGGGGTCCCCGGGGCGGGGCGGGGCGCGCTGTGT
CGCGGGTCGGAGCTCGGTCCTGCTGGAGGCCACGGGTGCCACACA
CTCGGTCCCGACATGATGGCGAGCATGCGAGTGGTGAAGGTAACC
GCGTATAGGATGACCCCTTTCTCTTGGCTCCCCTCGCCTCCCGCA
TTCCCTCCTCCCTCCATCCCCTCCGCGGCTCTCCGAGCACATTTA
TTTATTTGTCTGGGCACCAGGGCTGGCTGAATCCCTGTCCTCCCC
GCCCTCTACTCAGAGGAGGACTGGCCTGCAAGAGTGGCCAGAGCT
GGGGTTTTGGGGGTTTGGAGGCCTCTGCTTGAGCCGTTCGCGTGG
TGAA

In some embodiments, the intronic domain that promotes trans-splicing primarily in the brain comprises or consists of sequences from the gene UBE2L6. In some embodiments, the sequences from gene UBE2L6 comprise or consist of:

(SEQ ID NO: 243)
CTCCAGTCAGGGCGACAGTGAAACTCTGTCAGAAAAACAAGAAAC
AAAAAAACTGTGAGGTACTGAGATCACAAACTGGTGCTCAAAATA
TACTGATTTCCTTTCTCGTCTGGACTAAACGGGGGTAGGGAAGGG
TGAGAAGGAGGGGATCGGCCCTCCCAACAAGTCTCATCTTCCCCA
GATGCTGCTTGGTGCTCAGGGGCTGCAAGTACTCTGTCCTTGCAC
AGAGGGTTGGGGACTCCAGCTGTGCATGGGGGCACACATTAGGCC
CACAGCTTGTAATGGACCAGTAGTAAAGTAAGGGAGGGGAAGCAG
ACTCTCTCTTGGAAAGGAGGCTCCTGAGTCCTTCTTGGGGCTTCC
CCCACCAACTGTTTCCCAAACCAGCATACAGTAGGCACTTAGTAA
ATACTAAACCTAGTAAAGGACTGAATGATGGTAGACCGAACCCCC
AAAGCAAAAGAGAAAGGGTCCAACAGCGCCTCCTTGCGGTGCTCT
GCCC

In some embodiments, the intronic domain that promotes trans-splicing primarily in the brain comprises or consists of sequences from the gene CPSF7. In some embodiments, the sequences from gene CPSF7 comprise or consist of:

(SEQ ID NO: 244)
GTTTGTCGGGAGGGAGCGGGGCGGGCCTAGCTGGGGGCGAAGAGT
AGGCCTGGGTTGGGGGCAGAGGCGGCTGGAGCTGCTTGACACCTG
GGGGCTCTGGCCGGAGCCGCCTTGGGGGCTAGGCAGGTGCAGCGG
ATAGGGGCTGCGGGCTTCGCCGCGGTCATTCTTCGCAACGCCGTC
CTAGGGCCACTAGGACCCGAGGGTGGGACTTTTCCGTCGCGCCAT
AGGCAGCTGCGGCGCAGACGCGGCGCTACCCGGGTCGGGCCCGAG
GGTGATGGGCAGCGACCCCCGGCGGCCTGGGAGAGCGGTGGGCGG
CGCGAGGCCGGGGGCGGGGCGGAGCGGGTGGGGCCGAGCTGGGGC
CGCCAGGATGCTGCGTCTCTGGAGATGGGAGGTTAGCAATCATTA
CCCTCTTCCCAGCCCAGGACCCTCGTGCCTTCTAATTCTTGCATT
TTTCCGAATCCCGCAGTGGCATCTTCCTTACTTTGTCCATCCTCC
GGAC

In some embodiments, the intronic domain that promotes trans-splicing primarily in the brain comprises or consists of sequences from the gene CPSF7. In some embodiments, the sequences from gene CPSF7 comprise or consist of:

(SEQ ID NO: 245)
CGATGACTGATCGTTTAAGCAGATATTTCCAGACCGGCACACAGT
AGGCTCACCAAAGAATGGAAGCGACCTTTATTTCCTGAGTTGCAG
GATCGGCCTCTCCCTGGGCCTGCCCTTGCCCTGCGCTATCGGGCA
GACGAACTCTCCCCAAGATAGAGACCACAGAGGAAGACAAAGGGT
AATCCCCGCTGCCGCCCGCCCCGGACGCTAGAACGTTCTCTCTCC
TCACCAGCGACGAAGTCGAGAACACTGTAGACACCGCCATTTTCC
CCACCTGCACCGGAAACCGGCTGCGCGGGCACTTCCGGGAACTCC
TAGCCGCCCGGCCAGCCGACCACGGCAGCCACGGAGGAGCTGAGC
CGGAAGGTGCGTCCCTGCGCATGCCGGAAGTGGCGGTGCGTGTGA
CGCCGGCGGCGGGGGGGGCGGGACCTGGGGCGGAGAGCGGTTCGC
GCGCTTCGGGCCTAGTCCGGACTCGCCGCCGCCGCCGCCATATTC
CCGG

In some embodiments, the intronic domain that promotes trans-splicing primarily in the brain comprises or consists of sequences from the gene BSCL2. In some embodiments, the sequences from gene BSCL2 comprise or consist of:

(SEQ ID NO: 246)
GGACCCTCCATTTTCCATATCCAGGAAAATGTGATGCGCCACAGG
TATCAGCGTCTGGATCGCCACTTCACGTTTTAGCCACAAGTGACT
CAGTGGAAGATCCAGAGTCAACAGAGGCTCGTCAGGAAGATGTCT
ACAGAAAAGGTAGACCAAAAGGAGGAAGCTGGGGAAAAAGAGGTG
TGCGGAGACCAGATCAAAGGACCGGACAAAGAGGAGGTAGGGGCC
CTTGTGGGGACAGTCAGCAGAAATATACTGGAAATAGGTGGGCTG
GCGTGAAGGGGGCGGGGGGATGGATTGGTTTTAAGAATTTCATTC
ATTTTGAATAGATTACTTCCCACGCCCGCAGCAGTGTGCAAAGAT
GTACACAACACTGGCCTTGCCTCTGAAGGACTTTATCTGGGGTGA
TTAGACGTCTACAAAATAGAAGACTGCCAAAAGCTAGTCTAGATA
ATGTGCTGTGGGGACTCTGAGGGAGAGTGTATCCAGTTAGGGTCA
GAGG

In some embodiments, the intronic domain that promotes trans-splicing primarily in the brain comprises or consists of sequences from the gene BSCL2. In some embodiments, the sequences from gene BSCL2 comprise or consist of:

(SEQ ID NO: 247)
GTCATACCCCAATATTTTGTTCCCCTCCTATCCTCCTCCTCCTCC
TCCAGTGGACTGTCTCATCCCTGTATTCCTCATGGAAAAAAATAG
GGTGGGATGAGGTGCTCCCCAAACTCCCTCATGGCCTGGGCCACA
GATCATTAAACCTTACACAAATGATTTAAGGCTTTTCAGCCCTTC
AAACCATGTGTGTTCCCAGCTCTGAAGACCTCAACCAACCCTGAT
GAGGAGGGCACATCACCCCTGTCTGCTCCAGTGAGCCCCAACAGG
AGAAAAACAGGCCATTACTTACCTGAAGCCACAAGCGGCAGTAGC
AGCCCTGGGTGTAGGGTCAGTGGAGGGTACCAAAACCTTCTGCCT
GAAATGGGTGCCCCACCCAGTAGGGAGTTGCAGCTGAAAGTATCT
GATGCAGAAGCAGTGACTATACGAAAGGAGGCCCCTGGGGAGGGC
CAGGCCAGAGGTCTGGTCTCAGTTTCTGCAGCTCAAGCACTGTCT
GGAT

In some embodiments, the intronic domain that promotes trans-splicing primarily in the brain comprises or consists of sequences from the gene GNG3. In some embodiments, the sequences from gene GNG3 comprise or consist of:

(SEQ ID NO: 248)
CTGCATCAGATACTTTCAGCTGCAACTCCCTACTGGGTGGGGCAC
CCATTTCAGGCAGAAGGTTTTGGTACCCTCCACTGACCCTACACC
CAGGGCTGCTACTGCCGCTTGTGGCTTCAGGTAAGTAATGGCCTG
TTTTTCTCCTGTTGGGGCTCACTGGAGCAGACAGGGGTGATGTGC
CCTCCTCATCAGGGTTGGTTGAGGTCTTCAGAGCTGGGAACACAC
ATGGTTTGAAGGGCTGAAAAGCCTTAAATCATTTGTGTAAGGTTT
AATGATCTGTGGCCCAGGCCATGAGGGAGTTTGGGGAGCACCTCA
TCCCACCCTATTTTTTTCCATGAGGAATACAGGGATGAGACAGTC
CACTGGAGGAGGAGGAGGAGGATAGGAGGGGAACAAAATATTGGG
GTATGACCACAGCCTTGTAAAGGTTGGTGTGGAGTGAGCATGGAG
GGGGGCCTCCAGCTGAGACTGTGCTCTGAGAGGTCCCTCTTTTTT
CCAG

In some embodiments, the intronic domain that promotes trans-splicing primarily in the brain comprises or consists of sequences from the gene GNG3. In some embodiments, the sequences from gene GNG3 comprise or consist of:

(SEQ ID NO: 249)
TAGGTGGGACCCTGGCTGGCTCCCATTAGTTGGCCAGGCTGTGCT
GTGCTGCAGGTTCCCAAAGCTCAAGATAAGAGATGCGTTCTTTCC
TTCACTCCCTGAGGTCAGGGGAGGAGTCTGGGGCTCTGTAGAGAG
AGCTGTCCCCAGGCCTCTGGGGGGGATGAGGGAGAAGACAAGTCG
TAAGGAGCCCTGGAGATGGCAAAAGAGAACCCATTTGGGGAGGGA
GGCTGCAGTCCCCTTCAGAGGTCCTGGCTAACAATACCCTTCCTG
GCTGTGTCCCAGGTGTCCAAGGCAGCAGCAGACCTGATGACTTAC
TGTGATGCCCACGCCTGTGAGGATCCCCTCATCACCCCTGTGCCC
ACTTCGGAGAACCCCTTCCGGGAGAAGAAGTTCTTCTGTGCTCTC
CTCTGAGCTCCCCTGTCCCTTCTCACAACTCCTCCCTTTTCCCTC
TCCTGGGCCCTTCCTTAGGTCAGTAATTGTTGTGAGCCCCTTAGG
CTCC

In some embodiments, the intronic domain that promotes trans-splicing primarily in the brain comprises or consists of sequences from the gene SLC3A2. In some embodiments, the sequences from gene SLC3A2 comprise or consist of:

(SEQ ID NO: 250)
ACTTTCTTTATATACTTGGCCCCCTGCATTTCAGGGCTCCACCGC
CATCTGTCTGTCTGAGTGTAAAGTGGACCTTATTGGCATCTGGGC
TGCCAAACTGCTTTTTTTCCTTTGCTTTGCCCAGCCTTCTCTTGA
AGGCTAAATTCTTGAAAAATTATATTCTTTGTGTTCCTATAGTAA
ATATTAAGCTTTATATCAACACATATGGCAGGATCTGAGGCTCTT
TACTCACCTCTGGTCCATTTCTCTAGTCTTTTCTCTGTAGTCTTT
TTGTTTTCCTGTCCTCCATCTCCTAATTTATTAGTTTTATTATTA
TATTTTATATTATTTTTTTGAGACAGAGTTTTGCTCTTGTTGCCC
AGGCTGGAGTGCAATGGCGCGATCTTGGCTCACTGCAATCCCTGC
CTCATGGGTTCAAGTGATTTGTCTGCCACAGCCTCCTGAGTAGCT
GAAATTACAGGCACGCACCACCATACCTGGCTAATTTTTGTATTT
TTAG

In some embodiments, the intronic domain that promotes trans-splicing primarily in the brain comprises or consists of sequences from the gene SLC3A2. In some embodiments, the sequences from gene SLC3A2 comprise or consist of:

(SEQ ID NO: 251)
TGAGCCACAGTGCCTGGCAGTTTGTTTTATTTTGGTTGAACAGTT
ACAAGTCTTGGTTTGTTTTCCTGCAGCAACAGGTTTATCTTGATA
TGATACAGCGTTATTTAAATTCCTTTTCATTTTCCTGCCTTTGTC
TTCACATGCTAGTTTTTTTCAGTTGTTTCTTTTTTCTTCTTCCTT
TTCTTTTTTTTTTTTTTTGAGAGATAGTCTTGCTGTGTTGTCCAG
GTTGCAGTACAGTGGTGCGATCTGGGCTCACTGCAACCTCTGCCT
CCTGGGTTCAAGTGATTCTCTCACCTCAGCCTCCGGAGTAGCTGG
GATTACAGGTGCCCACCACCACGCCTGGCTAATTTTTGTATTTTT
AGTAGAGACAGCGTTTCACCATGTTGGCCAGGCTGGTCTTGAACT
CCTGACCTCAATTGATCTGCCTGCCTTGGTCTCCCAAAGTGCTGA
TTACAGGTGTGATCCACCGTGCCTAGCCTGTTTAGTTGTTTCTAA
GAAG

In some embodiments, the intronic domain that promotes trans-splicing primarily in the brain comprises or consists of sequences from the gene RTN3. In some embodiments, the sequences from gene RTN3 comprise or consist of:

(SEQ ID NO: 252)
CTAGTTTTTTTCCTTTTCAGATTGTGAGACTAACAGATTTATAGA
GGCAGTCTGTGCTCTGAATTGTTCAGCTTACAGATTGAAGTCTAT
CAACCCTTTATAGTATCAGGTTATTTGTTTTGTATAAATTATTTT
GCTTTTGTTGATTAGCTCTATCAAAGAATAAAAAACCTCCCAAGA
TTTCTCAATCTTACCGCCCTTCTTTATGCTTTCTCTGTACAATGG
AAAAGTAGACCAAAAGTATTTGTCATGAAAATTTTTTATCATTTG
CAGTTTGTGGCATGATATGGCTTTTCTGTTTCACTGTTGGCAGTT
TTATGCATGTCCTGTAGGATAACTTGATCGTTTTGTTACATATTT
ATGTGTGTATATATACATCCTGTGTGTGTATATATATATTTATGT
AATTTAAAAAATAAAAAGCATGCTGCTTGGCTTGGCTAGCTAATG
TGTAATTGTACCTGGTAAACAATTTTTTTCTTTGAAATTCTGATC
ATAG

In some embodiments, the intronic domain that promotes trans-splicing primarily in the brain comprises or consists of sequences from the gene RTN3. In some embodiments, the sequences from gene RTN3 comprise or consist of:

(SEQ ID NO: 253)
CAGTTTCCCAGAGCATCCTGCTTTTCTCTCAAAGAAAATTGGTCA
AGTGGAAGAGCAAATAGATAAAGAGACCAAGAACCCAAATGGGGT
ATCAAGTAGGGAGGCTAAAACTGCATTGGATGCTGATGACAGATT
CACTTTGCTGACAGCCCAGAAACCACCTACTGAGTACTCTAAGGT
AGAAGGCATTTATACATATTCTTTGTCTCCATCCAAAGTTTCAGG
AGATGATGTTATTGAAAAGGATTCCCCTGAATCACCATTTGAAGT
AATTATTGACAAAGCAGCATTTGACAAAGAATTTAAAGACTCATA
TAAGGAGAGCACAGATGATTTTGGTAGCTGGTCTGTGCACACTGA
TAAAGAATCATCCGAAGACATTTCAGAGACTAATGACAAGCTTTT
TCCACTGAGAAATAAAGAGGCAGGACGTTACCCAATGTCTGCATT
GCTCAGTAGGCAGTTTTCACACACAAATGCAGCACTGGAAGAGGT
GTCC

In some embodiments, the intronic domain that promotes trans-splicing primarily in the brain comprises or consists of sequences from the gene NRXN2. In some embodiments, the sequences from gene NRXN2 comprise or consist of:

(SEQ ID NO: 254)
GTAGGCGGCGCGGGCCCGCGGAGGAGGCGCGGTGGCGGGGCGGGT
GGCGGCCTCTGATAGGAGTGCGGCCGCACCGGGGCGGGGCTAAGC
GGCTCTGGGGCGGGGCTAAGCGGCTCTGGGGCGGGGCTACTGGAG
AGACGAAGCTTGCGAGGCGGGACTAGGGCAGGGGCGTGGCTAGGA
AAAGTCGGGATTTGTGGCGGCGGGGCGAGACCGTGGTGGTGCGAA
TGCGCCCCCTATGGGGGAGGGTGCAGGGCTGAGCTGGGAAGGTTG
CAAGGCCAGGGTCCAGTTTGGGGGTGGGGGGAGAAGTGGAGGGAG
GGTAGAGGTGAGGAGAATCTGATGCGATTGCGGAAAGGTTGAGTT
CAGAAATGAGGGAGACAGATGCAGAAACAGGAGAGAAAATCCAGA
AGTAATATAAAACGTGTCTTGGCAAGGAGACAGGGCTGTTACCAC
GGGCAGAGCCAAGGAAAGAACCGAGGTCCTTATAAAGAGCAAGTC
AGAG

In some embodiments, the intronic domain that promotes trans-splicing primarily in the brain comprises or consists of sequences from the gene NRXN2. In some embodiments, the sequences from gene NRXN2 comprise or consist of:

(SEQ ID NO: 255)
GCCTGCTTGGTACAGAGCATACCAGGGAGGAGAGGCTGGAGACTG
GGGACTGGGGAGGGGCTGGGGGTCAGACCTTCCTCTGGCAGAGGC
CGGCTTCTGTGGACTTCTCTGCTGATCTTGCCTTAGAGAGGCCTC
TGCCCTCCATGCCCCGAGCTGGTCCCCAGCTTGGCCCGATCACCC
CTGTCTGGGCCCAGTGGCCCTTGCACAGAGTAGGTGCTCTGTGAA
TACTTGTGAATATCCAGGACAAACCAAAAGGCAAGAGACAGGTGT
GGTTAGTGGAAGGCGCTCTGGGCCCAGGCTCTGCACGCACCTCCG
GCCTCAAGTTACTCCCTTGCATGTGGGGAAGGGGAGCGAGCCTGC
CCACCTCCTCCAGCGGTTGAGACCTGGGAAAGGTGCCTTAAGTGC
TGCGGAGCTGGGCTAGTGGTGGTCACAGGAGTAGCCACTAGAAGG
GATGAATGGTTGCTCCCTCTGGTCGCTGACCTCCACCTCCATGTC
CCCA

In some embodiments, the intronic domain that promotes trans-splicing primarily in the brain comprises or consists of sequences from the gene SF1. In some embodiments, the sequences from gene SF1 comprise or consist of:

(SEQ ID NO: 256)
GTTGGCATGCTTCAGATGATGGTTGGGTCTGATCTTTCTTGGTCT
CTTTTGGGGAGAGAATTTTAGTTTGTTTTTTTTTTTAAATCTGTT
CAGTATGTTTAGATCTCAGTCTGATCGGCTGCCCTGTCGCTTTGT
TTTAAAAAGCTTTTCCCTCTGATCCTGTACCCCTTGGGAAATAGA
AGGACTCTTTTGTGGCAAATGTAGGGAGTTTCTGCCTATGTACGG
GGAAAGGAGAACTTGGCTGCTATTGTATAGGTTGTTGAATTTTTG
ATAGATCCTTTTAAGAGTTCAGACCCTCAGTGTCCAACTGGCCAG
GAAAACTGAATGCTGATCTTGGCTAAACTTATGAAGACAAAGGTG
ATCTTGTTGAAGAATTGGGCTTTTCCCACTTGGTAGGGTGTAACT
AGGTTTAGTTGTGGTCCGGCCTCTCGTACAAAAGTGACTGGTGAG
GGGAGTGGAGGTGGGTGGAGCCCCCAGTCCCATCAGGACTGCTTT
ACAA

In some embodiments, the intronic domain that promotes trans-splicing primarily in the brain comprises or consists of sequences from the gene SF1. In some embodiments, the sequences from gene SF1 comprise or consist of:

(SEQ ID NO: 257)
CCCGGGCCGCCGCCGCCCCCGCCCGTGGGCTCGATGGGGGCCCTG
ACCGCGGCCTTCCCCTTCGCGGCGCTGCCTCCGCCGCCTCCGCCG
CCGCCCCCTCCGCCTCCCCAGCAGCCGCCGCCGCCTCCACCGCCA
CCGTCCCCCGGCGCCTCGTACCCGCCGCCGCAGCCGCCCCCTCCG
CCGCCGCTCTACCAGCGCGTGTCGCCGCCGCAGCCGCCGCCACCC
CAGCCGCCGCGTAAGGACCAGCAGCCGGGCCCGGCCGGCGGCGGA
GGAGGTGAGTCGGGCCGAGAGAGCGCGAGAGGCGTCGCGCGGACG
GGCCCGGCCGCCTGAGGGGCGCGCGGCGGCGGCGACGGCGGCTGC
ACGCGGCAGGGGCGCGCGTGCGCGCACGGGGAGGCCCGGGCGGAG
GCGCCCCTGGGCGCGCGCGGCCTGGTTCTAGGGCCTCGAGTGCGC
ACGCGCGCTGTGGGGGGAGGGCTGGGCGGGCGGGAGGCGCTCTGT
GCGC

In some embodiments, the intronic domain that promotes trans-splicing primarily in the brain comprises or consists of sequences from the gene TM7SF2. In some embodiments, the sequences from gene TM7SF2 comprise or consist of:

(SEQ ID NO: 258)
TGGGGGCGGGGGACTAAGATGGACGCCTGGGAAGGGAACTGGGAG
GCAGCGGGGTGCCTGGGGGCCGAGGGCTGAGGACGGGGTGCGGAG
GCGCACTCTGGGAATGCCGAGAGGGTCCCGCAGAGACGTCAGGGC
GCCCGTGCGGGCCGGCGGGGAGCTGGGGGGCTAGGGGCGGACGCC
CGACGTGATGGCCCTTCCCGCAGGCGCCGCGGCTCTGCTACTGCT
GCTGCCCGCCACCATGTTCCACCTGCTCCTGGCGGCCCGTTCGGG
CCCCGCGCGCCTGCTGGGTCCACCCGCGTCCCTGCCGGGGCTGGA
GGTGCTGTGGAGCCCACGGGCGCTGCTGCTGTGGCTCGCCTGGCT
CGGCCTGCAGGCGGCGCTCTACCTACTGCCGGCGCGCAAGGTGCG
GGCCCCGCTCGCGGACGCTCGGGGGAGGGAAGCGAATGGGCTCGG
CGAGGGAAAGGACGCCCCGGGCCTTATCAGAGCCCCCTTGGACCC
GCAG

In some embodiments, the intronic domain that promotes trans-splicing primarily in the brain comprises or consists of sequences from the gene SPTBN2. In some embodiments, the sequences from gene SPTBN2 comprise or consist of:

(SEQ ID NO: 259)
GTGAGGGACACAGGACCCCGGATGCCCACTCCAACCTGCTCCCCT
GACCTGTGCTGGCTTCTGCTTGGAGAAGACATGTCTTCTCTCTTC
CCACTGCACCAGTGGCCCCCAGATGTGAGCTGAGAGTGCCATTGT
CACCACACCTTTAGGAGGTGTCAGTTCCCCTGGGTGATGCCGAGA
CACCTTCTCCCGCTTTGTTTCTTGTCCCCACCATCTTCTCAAATT
CTGTTCTCCTCTTATCATATTTCATCAAATTAGAGTTGAGACATT
TCAGCTCTCCCTGTTTCCCTGGCCCTCTTACACGCAACCTTCTAC
ACGCTGAATGCAGCATTTTTTGACAGCTAGAGGAGCGGGAGAAGG
AGCGAAAGAGAAAGAGGGAGGAGGAGGAGCGGCGGAAACAGCCGC
CTGCTCCCGAACCCACAGCCAGTGTGCCTCCAGGGGACCTGGTGG
GCGGCCAGACAGCTTCTGACACCACCTGGGACGGGTGAGAGCCAG
GATG

In some embodiments, the intronic domain that promotes trans-splicing primarily in the brain comprises or consists of sequences from the gene SPTBN2. In some embodiments, the sequences from gene SPTBN2 comprise or consist of:

(SEQ ID NO: 260)
TGAGCTGAGATTGCGCCACTGCATTCCAGCCTTGGTGATAAGAGC
GAAACTCCGTCTCAAAAAAAAGAAAGAAAGAAAGAAATCACCTGT
CCCCCTGGATGACTCCCCAGGGGCCGTGGAAAGATCTCACATCCT
GGTGCTAACTCATATCTCTGCCCCCCGCCCCCAGATCTCAGAGAA
GCTGTCTCAGCTGCAGGCACGGCGCCAGGAGACAGCTGAGAAGTG
GCAGGAGAAGATGGACTGGCTTCAGCTGGGTGAGCTGCCAAGGGG
GCCCCAGGCCCTGTGGGGAGTGGGGGGCATCCTGCACCCTGTGGG
TTCCAGAGTAGGTGAGACTAGGAACCCTGGGTGTGAAACTCACGA
TGCCCAATCTTCGTGCTGCCTGGCACAGCTCTGGGGCAGTGGCTT
TCTCTGTGTCCCTGTTCTTGAGGTTGCCATGGCCACTGTGCCCTG
CCAGTGGCCACTCTGACCCACCATCTTCCTGCAACCCCCGATCCT
GCCA

In some embodiments, the intronic domain that promotes trans-splicing primarily in the brain comprises or consists of sequences from the gene PC. In some embodiments, the sequences from gene PC comprise or consist of:

(SEQ ID NO: 261)
GTGAGTGTCCGCCGCGCTCCGCACGGTCCTCTTCCCGCGGCGGGT
CGGAGCGGGTGGACGTGCGCCGGCACCGCTGCGAGGGACGCTTAT
GTAAGGGTCGCGCGCGCAAAGGCGGCGCGGGCTCCGCGAGTGACG
GCTGGAGGAGACAGCGACGGGCTGTGCGCGGTGCGCGACGCGACG
CAGCGGACGGCGCCCCTCCCCCTCGCGCGGGGGCTCGGCCGGCTG
GACCCGCCCCCGGACCCGCCCCCGGCTGCAGCCCCCGAGCGCCCC
TCCGCAGCGTCCGCCTGCGGGCTGCCTCATTCTTGCTCTCGGGCC
CATTTTGCAGATGGAGAAACTGACTAAAGCGCCTCGCGCGAGGTC
CCTGAGGGTTCGAACCCAAGTCTGTCAGGATCGGGAGCCCAAGTT
GTCCCCATCGTACTGGATGTGGCTCAGCTCTGCAGGAAGCCGCGG
CGTGTAAGGCGAGGGCTTTCCTTGTTCCTTGCTCTCGCACTCCGC
CTCC

In some embodiments, the intronic domain that promotes trans-splicing primarily in the brain comprises or consists of sequences from the gene PC. In some embodiments, the sequences from gene PC comprise or consist of:

(SEQ ID NO: 262)
TCTTCTCCGCTAGCCAGCGCGTATCTGCGTCTAGCCGGGATGCCT
GAGCCCCAGTGCGAACTGTTGTGCTTGCAGCTTCGGGAGACACGT
GCTACTCGGGTGAATGAATCGAACGTCCCCACCCCCGCCGACAGC
CAATAACTGCGAGCCACAGCCCGGCCACTTCCGCCTATTGCGGGC
GTCGGCTAGGGTCCGGCGGCCCACGTGAGGCTCCGGAGACAAGGG
AGTAGGCGGTGGTGCGCCAGGGGCGGGCTCTCCCAGCCTCGCCAC
TTATCCAGGCGCTCGCCGGGGACGGGAGGGGCGGGGCTGACGTGG
GGCGCCAAGGCTTAAACGTGACGGACAGGCGGGCACAGGGAGGAG
TCCAGAGCCTAGGGGCGAGGAGTGACGGAGAACACTGCCCAATAA
CGGGAGGGGTTGGGCTGTCTGGGCCAATAGGAAGTCCGTAAGGCG
GGGCCGGGACTGCAGCAAGTTCGGTTGCACGGAGACCGCAGCTGT
TCTC

In some embodiments, the intronic domain that promotes trans-splicing primarily in the brain comprises or consists of sequences from the gene ANAPC15. In some embodiments, the sequences from gene ANAPC15 comprise or consist of:

(SEQ ID NO: 263)
GTTAGTTCATCCATTCAATCTTCCATCTACTTATTTATCCACTTA
TTCATGCCTCCTAGAATGTCTTACTGAACCCTGCTTGCATATGCT
ACAATCCCTCCACCCCGACATTTGGCTGTGATTCATGAGAGGTCG
TTAGTGTCCTAAATTTCTGACACTGGGTGGGTGTGGTATGGGCCA
TCATAAAGGGTCAAAAAGCAGGTTTTAGTGTGTGGAGGAGGTAGG
GATAGTTTCAGTAGAACCCTGCTACCTCTAATTTTGTATAATTTC
ATAATGCCAGTCTGCATTCTGGATTCCTATCCCTACACTTGACTC
CCATTCTCATCCCTCCTTGAACAGCCTGCAAACAGCTTTGTTGAT
CCATTATTTACTTAATTATCAGAATAACTTTGACAGTGGTATAAT
TGTTATTATCCCCATTTTACAGATGAAGACACTGAGGCTCAGAGA
GGTTAAGTGACTCAGCCAAGGTCAAACAGCTAGTAAGTGGTGGAG
CCAG

In some embodiments, the intronic domain that promotes trans-splicing primarily in the brain comprises or consists of sequences from the gene ANAPC15. In some embodiments, the sequences from gene ANAPC15 comprise or consist of:

(SEQ ID NO: 264)
TGGGTTCTTCCGCTCAGGCTGGGTGAAGCGCTTCCGGGTCGCCGC
CGGCAGCAGCCTCCCGGCGCGGTAAGTACAGACGCAAGGCTGCAA
CCGTCTCCTTGCCCCTTCATTCCTTTTTCCCCCAGAGAAACCCAT
TTCCTTTGACCCGACTCCCCAGGAGCTCCATATTGAGAGTCCTTA
ACCCTTAAAGAGCTCCAGCTCATTGGCCGCTTCCTCGAGACTAAG
CCCCACCCCGTCACACAAGGTCCGTCATTCATTGGTGTCACTTCC
TGTCACCCAGCATTTCTTTCAGTTAATTGGTCCTACCCAGGCCCC
GCTTTTTTCGCGTTTAGCAACCCTATAAGGCCAAATTTCTGTCTG
AGACCCGCCCTGCTTTCTTTCTGTTGGTCAACTGCATGGTCATTA
TCGTAATGAGCAAAGAAAGGGCGGTGGCTTTGCGACGGTGGGTTG
GGCTTTCGGAGCCGAGATCGCTGGTCTGTCAGTACTTCTTGGACA
TGAG

In some embodiments, the intronic domain that promotes trans-splicing primarily in the brain comprises or consists of sequences from the gene MAP6. In some embodiments, the sequences from gene MAP6 comprise or consist of:

(SEQ ID NO: 265)
GCAATGTTTCTCTCTGTCTCTGTTGGGCAGGGGTGCATACATGTG
TATAGGTGGGCCTGTCAGGCTCTGCATTCACCCGTCTGGACCAGA
GGGGTCTGAGAAGCACCAGTTCCTGTGAGTTGTCAGCATGAGATG
GGAGTGGGTAGGCGATGCTAGATGGAAACAGGACCCCATCACGGA
GGTGGGCAGGATAAGGCAAGGGTCAGATACCATGACAGCCTCACA
AGAGGTGCCAAATCCCAGCAGGCTGAGCCCTGGGTAGCTGAGTTG
AAGCCTGCAGATGAAACAGGAGCAGGGACCCACCTGGCACAGGGG
AATTCTGGATGACAGTTGTTTGCCAAGAGCTCAGCCTTAAAGCTC
ATCCAGGGGTCAAACTTCTTGAGGAAACCAGGCTCTGATCATAAG
TCTCAGGACACTGTATAACCTAACACCTGCTAATCCTCATTCAAT
ATCCATGACAGCTCAACTGAGAGCTTCTAAAGAATATACCTAGAG
CCAG

In some embodiments, the intronic domain that promotes trans-splicing primarily in the brain comprises or consists of sequences from the gene MAP6. In some embodiments, the sequences from gene MAP6 comprise or consist of:

(SEQ ID NO: 266)
CTTAACAAGGGGGTCAGGGAGTCAGGGAGGCAAAAGAACTTTGAA
GGCCCTGAGAACAAGAAAAGAGCACCCTGGCCTGTGGGGGCATCT
GGTGGGCAGCTGAGATGGGGCTCCCAGGATGGGCCCAACGATCCT
AGAAGAAAAAGTAGGGACCTGGATGGAATGTGGGGTGGACGGGCT
GCTGGGATGCCTACTGGATGACTCCGAGTCCAGCTACAGTACTGC
CATTGTCACCAGCGTGCCACCCCCTGTGCCACGTGCAGGTTATAA
TCAATTGGCAAGCACATTGAATGACTGATTCCTGAGCCTTCACCT
CACCCCCCTCCCTCACCCCCGCCCACATTCTGTACTAAGAACACA
GAAATCATATTCTCCTGGCTCTCCAGCAAGAGCTGCCCGGCCCAG
GCTCCACTAGGCCAAGACTTATTAGGCAATCTGTACTCTGCCCTG
TCCCTGCCTGAGCTCTACTCTCACAGTGATCACCTCTCTCTCCTT
TGCA

In some embodiments, the intronic domain that promotes trans-splicing primarily in the brain comprises or consists of sequences from the gene GAB2. In some embodiments, the sequences from gene GAB2 comprise or consist of:

(SEQ ID NO: 267)
GTGAGTTTTGGGAAACAAAGAGTATGGCGGGCAAGTACCTAGCAG
GTTCTGAATATTTGTTCTTTAGGAGTCACCATTTAAACAAGACCC
CAAGAGTGACATTAGGTGTAACCAAAGTGAAATTGTTGACTACTT
GATGAGATTCGTGAGGATAGGCATTAAATGATATCTTTTAAGAAA
CCATTGGCAGTCCTGGAGCTGATGACCCTAGTGCAGGATCATTAT
GGTTGCTTCTTTCATCATCTCAATTTGATGACATATTTGAATTGA
ACTTAAACTATACCTAAGCCTTAATCCCAGGGTCTACTAGTGATT
CAGCTCAGCACATCTAAAGGCAATGATTATTCAAGAGGATTACAT
TGGTATCTCCTGTCACACCAGCATTTAGTATTCTATACTTATTCC
CTAAATATATCTCGTTGTCATTCTCCGATATTTACCATTTACCAA
TATTACCAATATGCCTAGAGCATAGCAATTGCTCAATAAAAGTGT
ATTG

In some embodiments, the intronic domain that promotes trans-splicing primarily in the brain comprises or consists of sequences from the gene GAB2. In some embodiments, the sequences from gene GAB2 comprise or consist of:

(SEQ ID NO: 268)
AGGAGGGAGGGTTTATATTGTATTTTAAAGATCAAGGACAACAGC
CTAGAGGAGGTGGCATTTAAGGTAAGCCTTGAAGAAGGAGTTTAA
TTTCAACAGAGAGTAAGGCTGGGAATAATGCGATGTGAGAATGAT
CTTGAGAAGAGGCACAGAGGGTAGGTTCAGGGCAGATCCGTTGAT
AGCATTCACTAGAGTGTAAGCAAAAGGTGAGGAAAACTGAAATTG
TAGGTTGAAATTTTTGTTTCTGCGAGTATACTTATTTGCATTTGC
ACAGAAGGTGGTGATCCTTATATTTATTTCTGCTGTGCATCAATA
GGGGAGGTGGAGATAAATAATTTCATTGTTTTGGCTCTGGCCAGA
ATTCATTAGGCACAGTTTGTTGTCCATAGGGAGGGGAACACAACA
CACACTGGCCTTTGTCTCCCTTCCACACAGGCTGGTGGAGATTCT
GAGGCATTTTTAAACATTGCACTTAGCCATTCAGATGATAACTTG
GATC

In some embodiments, the intronic domain that promotes trans-splicing primarily in the brain comprises or consists of sequences from the gene BACE1. In some embodiments, the sequences from gene BACE1 comprise or consist of:

(SEQ ID NO: 269)
GTGAGTCTAGGGATGTTTTGCTGAGCAGCCTGAAAGCAGACTCTA
GGATTCCCCTGAGATGAGATAAGGTGGTGTTTGGGAGTAAGTTTA
ACTTAGATCCTTATACCAGTTGTGGTTATGCATCCACAGATCTCT
GCCTTCTTTCCACAGGTCCAGCACATACCGGGACCTCCGGAAGGG
TGTGTATGTGCCCTACACCCAGGGCAAGTGGGAAGGGGAGCTGGG
CACCGACCTGGTAAGCATCCCCCATGGCCCCAACGTCACTGTGCG
TGCCAACATTGCTGCCATCACTGAATCAGACAAGTTCTTCATCAA
CGGCTCCAACTGGGAAGGCATCCTGGGGCTGGCCTATGCTGAGAT
TGCCAGGGTGAGAGGAACAGGGCTTGGCCCTACACACTCTATGCA
CACTGCACTATACAGAGAAGGAATTAGGAGAGAGAAGGGGAATTA
GCAGAAGGAGGGCAACAATCAGTTTAAAGGATTATTTAACAGGGG
TATA

In some embodiments, the intronic domain that promotes trans-splicing primarily in the brain comprises or consists of sequences from the gene BACE1. In some embodiments, the sequences from gene BACE1 comprise or consist of:

(SEQ ID NO: 270)
AAGAAGAAGGGATGAGAGGACACCAATATCTGAGACATCAGTGTG
TGCAAACGTGAGTAGAGGCTTAAATCCTATCCTTGGACTTGTAGG
CAGCTGCAAAACGCACCTTCCCTGTTTCCTCTAATGTCCCAATCT
AAAAGAAGCAGTCAGGGAGGAGGGTGCAGGTAGGATCGATGAAAG
GGCATTCACTCCAGTTCCTCGTTCCAGTTCCATCACTGCCCCCAC
CACACACAGAAGCCCTGGTTAGTGGTGTGATTAAACACAGGTGGG
GCTGCCACATGTTGAGCACGTTTCACAGAAGTGGTGTGTAATACC
AACCTGCTTTTCTGAGATGCTTGAACAAACCCCTTCTCTAGAACT
TGCTGTAGAGGTTCCACATTCACCCTGCCTGGCACTTTCTCTACC
ACTCGGATGGTACCACTAGGTTCCAAGGCAGTAAGAGAGTCAATG
GTTCCTTTTCTCGGGGAGCTTGACGGAGCCAGAGGGCGGAAGAGA
TGCC

In some embodiments, the intronic domain that promotes trans-splicing primarily in the brain comprises or consists of sequences from the gene FXYD6. In some embodiments, the sequences from gene FXYD6 comprise or consist of:

(SEQ ID NO: 271)
GTAAGGACCATCCCCCCAGCTCCCCACCTGGCTCTAGCTGAAAAT
GACAACTACAACTGGAGCTGGCTCCTGCGCCCACGACACAGCCAT
TCATGCTTTTGGTGCACATTCCCCTTAATTACCAAGGCTCCCAAT
TAACGTGGGCTGCTGTGTCTGGTGCTGGGTTATTTTCTCCTATTT
TTCCGTTCCTCGGGGTTTACTATTGCAGAGAGACACCAGGTCTGT
CATTGTGCCTGTCTTGCTCCCTTCCCACCCTAACCAGAGGGAAAC
CGAAAAGCAACAGCTGATGGCAAATCCGCTCCGGCGCTTGCACGC
CTCCTCGGCGGCAGTCTCCTGTTGCTTCTCGGAGCTTGACTGCCC
TTTGGAAAAATCTCTCCGCCCCTCGAGAGGAGAGAGGAGCAGAGG
ACGCGCGCGAGCACAGAGCGGGAGGACGGACAGGGAGAGGAGGAG
AGGGGGTCTGCGCGCGGCCGCTACCCAGAAGCCAGCGGACGGCAG
CACG

In some embodiments, the intronic domain that promotes trans-splicing primarily in the brain comprises or consists of sequences from the gene FXYD6. In some embodiments, the sequences from gene FXYD6 comprise or consist of:

(SEQ ID NO: 272)
TATATTTCCTGCCTTGGGGTTTTGAGACCTGACCTTTTATTAAAA
TAATTAGATCATCTAGGGCAGAGCTGGAAGAGACCTCAGGATCAT
CTAGTCCATCTCCCTATTTTCCCAAATGAGAAAACAGGCCCAGAA
CTGAGGGCAGCAGAGGCAGGGCTAGGTTTCTTCCTAGCAGTACCC
ATCATCCCCGGCTTGGGGTTTGTAAGGAACTCTCCGTGGCCCTAC
CTAGGCTTGGACAATCACAGCCTTTCTCAGTAGCTCCTCCCTCCA
CCCGGCTCGGCCATACACACACCTTGACTGAGGCTGGACAGCCTC
CATCTCAGCATGGCTCCTGGCTACTGACACTTGTGCCTGGCCTCT
GCCCCTTCCTACAAGCTCTGCCCCTAATAGTAAGAGCCAATTTCT
TGAGCACTCACTATGAGCTAAGCCTCCCATGGGGTCAGTCTCTAA
TCACCATTTTCCCACGTGAGGAAATAGACTCAGGAGCTGAGTGCA
GAGA

In some embodiments, the intronic domain that promotes trans-splicing primarily in the brain comprises or consists of sequences from the gene PHLDB1. In some embodiments, the sequences from gene PHLDB1 comprise or consist of:

(SEQ ID NO: 273)
CTGGGTACCCTCAGTAGATGTTCTGGAGATGTTGAGAGCATGTTG
TAGGGGCTTCGATTTGGGAGGTTCAGTGGGAGCCCGTGGAGGTTT
CAGGCATCTAAGGCAAAAAGAAAGAGGGGAGAGTTTGGGGGAGGG
AGAACGGATTGTCAAAAACACCCTCTCAAAAGCGTGTTAAAGCCT
GGGAGTGGAGGGTGGTCTAGGAGAGTCGGGAGGGCAGGGTGGGAG
GAGAGGGGGATGTGTGGTGGTGGTGGTGGTGGTGGTGGTGGTGGT
GGTGGTGGTGGTGGTGGTGGTGGTGATGGTGGAGAGCCCAGCTGC
AAAGATGAACAAAGCGGGCGCTGTGCAGGCTGGGGGAGGTGCTGC
TGTGGGAAAGGGTGGGTGGGGGGACCTTGGGCTCCAGCCCCACAA
GGGCGTCTTCTTTGGCTGGCCAGGCGGACGGGACGGGGGACGGGG
GCGGGGACTGGCGGGAGGGGGGCGGGACAGGCTCGCTCTCCTCCT
ACTT

In some embodiments, the intronic domain that promotes trans-splicing primarily in the brain comprises or consists of sequences from the gene PHLDB1. In some embodiments, the sequences from gene PHLDB1 comprise or consist of:

(SEQ ID NO: 274)
TAAGAGCGCCCCGTCCCACTACACTTGCGGGTCCCATTCGCTGCG
GTGCTAGGACTGGATAAGGGGAAGTCCCCGGGGCCTGGCGAGAGC
CCTGAGATCAGCTCTAGGCTAGGGAGCTCGGCAGAAACCCGTGGG
GGAGAGAGGGCACCCCAGGTGAGTGCGTGGGGGCGAAAGGAGCGT
TATGGGGAGTTTTCCTGTGAGCCTCTTGCTTTTGCCTTCCTCGGC
CGCTGCCAACGCCACCGCCAGAGGGGGCCGTGACGCTCTCCCCCC
CTTGGAGAAACTCTGATCCTGGCCCTATCCAGACTCTCCCGCCGG
CACCCAGGCGGCCGGGCGGACACTCGCGGGGTATCGGGCGGCGAG
CGCGCGCGCGGGTTTTCGTCCTCCAGCGCCCCACACCTCCCCCTC
TCCGCCCACAGCAGGACCTTGGGGCGGGGGTATCTCCTGTGACGT
CTCCCCGCCTGTCCCCAGGCCGGCCGCCTTGATGGACCAGGAAGG
GATT

In some embodiments, the intronic domain that promotes trans-splicing primarily in the brain comprises or consists of sequences from the gene NRGN. In some embodiments, the sequences from gene NRGN comprise or consist of:

(SEQ ID NO: 275)
ACCCAGTGACCCCACAAGAACCCCCCTGCTTCGCCCCCAGGAGAA
CGCCTGCTCCAAGCCGGACGACGACATTCTAGACATCCCGCTGGA
CGATCCCGGCGCCAACGCGGCCGCCGCCAAAATCCAGGCGAGTTT
TCGGGGCCACATGGCGCGGAAGAAGATAAAGAGCGGAGAGCGCGG
CCGGAAGGGCCCGGGCCCTGGGGGGCCTGGCGGAGCTGGGGTGGC
CCGGGGAGGCGCGGGCGGCGGCCCCAGCGGAGACTAGGCCAGGTG
AGGCGGGCGGCGCGCGGCTGGCTGACAGCTGCCCTTCCCCAGCCC
TCCCCAGGAGCAGGGGGAGAATAAGGGCGGGTTGGAGGTGCAGGG
GGCGTGGAGGGAGCTAAGGGTTGGGGGATAGAAATCCGAGATGGG
AGGTGGGTGGGAAGAGGCTGAAGGTTGCGCGGATTCCAGGAGCTC
ACCTGTTTCTCCCTCTGCATCCTCCCTTCTGCCCCGCCCTGGACC
GAAG

In some embodiments, the intronic domain that promotes trans-splicing primarily in the brain comprises or consists of sequences from the gene NRGN. In some embodiments, the sequences from gene NRGN comprise or consist of:

(SEQ ID NO: 276)
ACTGAGCATTTTCAAAGGTAATGAACACGGCCCCTAGGCGGCGGG
GTGGGACCCGTGAGTGGGAGAAAAGGATCGAATCCCCAGCCCTTC
CCTCTGCCCACCCTCACCCCTCCCAGCCGGGGAAAATGCACCCTG
CCCCTGCGGGTTTTCTGATACATCCAGACGCCCGAGGCTGCGACG
GGGACATACCTGGACCAAGACATGCGCAGCCACCCCATTTGGCTG
CACAAGCCCGGCCAAGGGGCAGAAATACATCGCGGCGCGCACACG
TACCTGCCGCGGGGCTCATTTCAGCGCTTTGACTCGGGGTGGGGG
TTCTGGGTGACTGGGGCCCTCTGGCACTCACCGCCCAAAGAATCC
TGGTACTGAAAGGGGGAAGGGTGGGGGGAAGAGGAAGTAGCAGAA
GAGGGCAGGTTCTCAGACCTTTTCTCTCCTCAGTTCCCGAGGAGA
GATGGATGCCGCGTCCCCTTCGCAGCGACGAGACTTCCCTGCCGT
GTTT

In some embodiments, the intronic domain that promotes trans-splicing primarily in the brain comprises or consists of sequences from the gene TPI1. In some embodiments, the sequences from gene TPI1 comprise or consist of:

(SEQ ID NO: 277)
GGGACTCAGTCCCTGCAGTTCCTGCCAGGCCTTGCCAGCCGGGGC
GAGGGTTGGGATGATCCTGGCGGCCTATGCCTGTGTGGGCTGCCC
CTCCCGCTGTGAACCCTGCATTTGTCCCGCAAGTTTTCACTCAGG
TAGACTCCCTGGGTACAAGGGTGCCTGCTCAGCAGTCGGGCATGA
GCTGCTCCGATGGGCGAAGGAGGTTGTCTATCCCACAGTTGGAGA
GGGGCCCTCTCTGCCCCAGTGGGCGATCTGGGCTACGGCCAAGTT
GCCACCAGCTAGTTCCGCTTGAAAACCACTTCTGGCCCCGTGGGG
GACTCAAGTCGCCAAGCGAGGGTTCCCCTGAGCGCCGGAGCTCAC
AGGTCTCGCCTTGTCCCGAAAGCCCCGCAATCGAGGCGGAGGCGA
CCGAGCCCCCGACTCTCCTAGAACGTTGCCACAAGAAGGGGGAAC
GTCGGAACAGTGCATCATCGGGCGGCGGCCGGGGCGGCGGCAGGA
GGGC

In some embodiments, the intronic domain that promotes trans-splicing primarily in the brain comprises or consists of sequences from the gene TPI1. In some embodiments, the sequences from gene TPI1 comprise or consist of:

(SEQ ID NO: 278)
CGCAGACACTGACCTTCAGCGCCTCGGCTCCAGCGCCATGGCGCC
CTCCAGGAAGTTCTTCGTTGGGGGAAACTGGAAGATGAACGGGCG
GAAGCAGAGTCTGGGGGAGCTCATCGGCACTCTGAACGCGGCCAA
GGTGCCGGCCGACACCGGTAAGCCCTCGCCGAGGAGGGGTCTGGC
CGGGCCGGGGCCGGGGCCGGGGCAGGAGTGGCAGCGCCCTCTCCC
GAGGCCCCGAGGCCCCGAGGCCGGTATCCGCGCGGACCTGATGCA
GGGCTGTGGGACGAGGGCCGCTGGGGTCCGGGCAGGGGCCTCGCA
GCCGCAGCCCCGTCGGTGCGTCGAGGGGGCAGGGCGGAGCACATG
ATGCCCCTTGGACTATGGGGCAGGTAAGGACGTTTTGGGTCTCCT
GGAGGAAGGTGGCCCCGGGGCGCGCACTGGGGCTGTGCCCGCCAG
GCGACGGGGTTAGGAGCGGAGCCCGAGGCTCTGCGGGAGACCGGG
GGAG

In some embodiments, the intronic domain that promotes trans-splicing primarily in the brain comprises or consists of sequences from the gene ZNF385A. In some embodiments, the sequences from gene ZNF385A comprise or consist of:

(SEQ ID NO: 279)
GTATGAAAGGGGGTGGTGTCTGGAGGGAACAGGAAGGGATCCTGG
AGACCTTCTGCTCTAGTAAAGTCAAGGGAGGACGGGCCAAGCACG
CAGCCTCCTCGGTTTGCCTTTGTTCCTGGGAACCCCTGTTGCTGT
CAGGGGGTTACTGGAGCAGTGAAGGGCGGCCAGAACTCCCCAGCT
GGTGCACAGCAGGGTCGCAGAAGGGAGCTGGGCCAGTGGAAGCCC
AGAAGGGCTCTGAGGACCTTTGGGCTGCCCCTTTGGAGTCTGGTG
GAGTTCCTCCCGTGACTCTGAGAGGAGGAGGAGAAGGTTTCAGAG
AAGGGTAGTTGAATGGCTGGCCTGGACTTTCATTTCCCCTTGGGG
TCAGACCCAGGCATTCCCCGCCCATAGGCCAGGTCGAGGTTCCTC
CTGCAGAACTGGACCTGTAGCTGAGCATTCCTGGTGAGACAAAAG
GGCATGGGGGAAGACTGAGGAAGGTTGGGAGAAGGGGCTCTGAGA
ATGG

In some embodiments, the intronic domain that promotes trans-splicing primarily in the brain comprises or consists of sequences from the gene ZNF385A. In some embodiments, the sequences from gene ZNF385A comprise or consist of:

(SEQ ID NO: 280)
ACTGTCCGATTCCTCAGTCACCTATTCCTGCTGCTACCACTCTCC
CATCCTCTCACTCACTTTCATCTGTCCGTCTCTCTGTGTGTGTGT
GTGTGTGTGTGTGTGTGTGTGTGTGTCCTCTCTCCACCCCATCCC
AACCTTGAAGGTGACAGAGGAGGGAATAGACACAAAGCATACAGA
CCCCCAGCTACTTAAGGGTCAAAGCCAAGGATCTTACCCTCTCCT
TTAGCCCTGAGAATTCTGAAGCCCAAACTCCCAGTAGCCTTGACC
CCCTGCTCTGTCTCAGTCACCCTGACCACCCATCCTGGCATGGGG
TGAAAGAGACAGACCCCTCCAGACCTTTCTGGACCAGCTCCCAGC
CCCCAGCTGTGCCTGAGCCCTCCCCACCCCCTCCCTGTCGCCTCA
GATTCCCCTAATCCCTGCTCCCCTGCCCTCCCCTCTGCCCCACTT
TTCCAGGATTCTGATTGGCTGATTCTACTGACTGGGCTGAGGGGG
AGGG

In some embodiments, the intronic domain that promotes trans-splicing primarily in the brain comprises or consists of sequences from the gene DTX3. In some embodiments, the sequences from gene DTX3 comprise or consist of:

(SEQ ID NO: 281)
ATGGGCTGCTGGGTTGGGGGCCATGGGAATTGGGAGAAGGGGTTT
CTCACTTTCCTTCCATTTTTCCGCCCTACCAGGTCACACGACCTA
CAAGTAGGGAGCAGGGAAAGAAGAGTGAGCCTGCATGCCAATTCT
AAGCTCTTCAGGATCAAAGTAAGCAAAAAGGAGGGGCAAAGAGTC
TCCCTCCCCTTTAAACAGCCCATTTTTCCCCAAGAGGTGGGAGGG
GCAAGTTGTTTCTATGATCTGGGAGAAATGACAGGAGAAACAAGA
CTGATACTTGGCTTAGATGATAATATAGCTCTGGAAACTTTTTTG
CTCTTCAGTGGGTTTTGGGTTTGGAAGATAAGGGGAGACAGAATA
TGAATTTGGGTGAAGGGTTGGGGATTAGGCTTTGAGAAAACAGAG
GTAATAGAGGGTTTGGAAGGAACCTGAAGTAGGAGGCACTGATAC
TTGTCAAATGGGGACCCCCCACCCTGAGTCCCTTTCACCCTGGGG
GCAG

In some embodiments, the intronic domain that promotes trans-splicing primarily in the brain comprises or consists of sequences from the gene DTX3. In some embodiments, the sequences from gene DTX3 comprise or consist of:

(SEQ ID NO: 282)
ATGGCAGCCTGTGGAGGCACCTGCAAGAACAAAGTGACTGTGTCC
AAGCCCGTGTGGGACTTCCTGAGCAAAGAGACCCCAGCCCGGCTG
GCCCGGCTTCGGGAGGAGCACCGTGTGTCCATCCTCATAGATGGC
GAGACTTCTGACATCTATGTTCTCCAGCTTTCCCCACAGGGTCCT
CCCCCGGCCCCTCCAAATGGGCTCTACCTAGCCCGGAAGGCTCTC
AAGGGGCTGCTAAAAGAGGCAGAGAAAGAGCTGAAGAAAGCTCAG
AGGCAGGGGGAGCTGATGGGCTGCCTGGCTCTGGGGGGTGGAGGG
GAGCACCCTGAGATGCACCGCGCAGGCCCACCCCCTCTCCGAGCA
GCCCCACTTCTGCCCCCAGGAGCTCGGGGGCTCCCCCCTCCTCCT
CCCCCCCTGCCCCCACCTCTTCCTCCTCGCCTTCGGGAGGAGGCA
GAAGAGCAGGAGAGCACCTGCCCCATCTGTCTGGGGGAGATCCAG
AATG

In some embodiments, the intronic domain that promotes trans-splicing primarily in the brain comprises or consists of sequences from the gene CORO1C. In some embodiments, the sequences from gene CORO1C comprise or consist of:

(SEQ ID NO: 283)
GTGTGTTTCCCTAATAGCGGGGCAGCTGGCGAGGGACGCAGTAAA
AAGGAGCGAGGTGCAGGTGGGGCCCCGCTGCGCCGAAATGACTGA
GGATGAGGTCAGACGTGGAGGGTGGGAATGCAGAAACCGAGCTCT
CCCGCCTGTCCCCACCGGCGCACGCTCCCCAGCTCTCCGTCTCTC
AAATGATTTGAATGTGTTGGGGGAAGTGGCAGGCTGCGGTGAGAG
CTGAGAAGAAAGGGGCTTCCTCTTGGAGGGAGGCGCTGGCCCTGG
CATCCTTTTTTGGGAGTGTTCAAAGGATTTTGAACGCTGGAAAAG
GAATCGTAGGCTTTACTGCATCACGATTTAGAAACACATCCTATA
TTTGGCCGTAATGTAGGCTGCCCTTGTTTTGTGGTGTCTAAAGTG
CTTGCTCTACATGTTGCAAACGTGGGATTCAGAGTGCGAAGCAAA
TTGCAAGTTGTTCATTTCCCCCGGCATGGTAAGATACAAAGGCTT
TTCC

In some embodiments, the intronic domain that promotes trans-splicing primarily in the brain comprises or consists of sequences from the gene CORO1C. In some embodiments, the sequences from gene CORO1C comprise or consist of:

(SEQ ID NO: 284)
ACAGGAGCCGGCGGGGCGAGGGGCAGGGCTAGGTGGAGGGGACAG
TGCTGGGTGGGCGCGCGGGGCGCGGGTTCACGGGCTGGGTTGCAA
GGGGTTAGGGCCGGGGGCTGCTGTCGGGGGCTCGGGGAAAGGGAG
TTGGGGACCGAACCAGGTGGCGTGGGGAGCGTGAAGCAGGGTCCC
TGTGGGGAGGAGGCTTTCGAGGTCCGGAACGCTCCTGGCCGGGTG
CGGAGCCGCGGGCCGAGGTGGGACGAGGCTGCCCAGGGAAGGCTT
TTAGGGGTCAGGGATCGCCTGCTTGGGCGAGGTGCTGGGAGGGAA
GGGCTGCTTCGGGCGAGGCGTAGAGCCCGGGGGCTCCAGGCCTGG
GGACTGGGTTTGGGCCGCCCCAGGGAACGAGTGGGTGGGGGCGGG
TAGGGGCCGGGCTCACGTGGGGCCGGGGCGAGCTGGGCCCTGCGT
CGGGAGGGGCGAGGGCGGCCGCCTGGCCTGCGGGTTTCCTGGCTT
TGGG

In some embodiments, the intronic domain that promotes trans-splicing primarily in the brain comprises or consists of sequences from the gene VPS29. In some embodiments, the sequences from gene VPS29 comprise or consist of:

(SEQ ID NO: 285)
GTACTTTTATGTTGATTACCAGTGAGGGTGGAAACTTCATAGTGG
TTTTTAGTTTTACAGATGTTTTGTCCACTGAGATCTGCATTGAAA
CAATTAATATAGGTCATACAACAAATACCTGCTGAAACTAGTCTT
TATAAAGAAGAATCTTACTTAACAATTTTAAAGTGTGCTAATTTC
ATAACCTTGTGTTACTTTGAGATTACAAACTATGTCCCGATAACT
CTGTGTCCTGGTAACGGTTCCTTTTCTTTGCAATAATTTTAATTT
TTGTTGCAAGATAAATCAAGCTTCTTTTGGTAATCATTTTAATTT
CATACATTTTGTTTGTTTGATTAATTTTCATTTACAAAGTCTGCA
GTGGCAAAACTATTTTGTTTTATCCTATTAGCTATTTAATGGTTT
CTTTGATAATATTAAAGAATAGTCATGCCTTTAAAAGCAGTTAAA
GGGCATGTACATTTATGCATGCAATATCTGTAAAGGAAAACATTT
TGTA

In some embodiments, the intronic domain that promotes trans-splicing primarily in the brain comprises or consists of sequences from the gene VPS29. In some embodiments, the sequences from gene VPS29 comprise or consist of:

(SEQ ID NO: 286)
TTAAAGGACTCTATCATCTATGTCTATCCCGATTTGTCAAAGTGT
GGAATTTGGGCGGGAACATGTTTCAAAGTATGACACGTGTTATGT
AACACTATTTCCCCATAACTTTGTCATCAGCAGGAAACCAGAGGA
TTCTGTCCTAGTAAGGATCCCTACTAATTTGAAATGATTGTGTGG
TCATTCATACAGTTATATCTTTAGACTGCTAATAGTCTTGAGTCT
TGGAGATAATCCACAGTACTTTATAGAATTAGGTCATCAATCATT
ATAAAGTACCATGTCTTACTAATGTTCTTTCTGGTACATTCAGAT
TGAACAGCTCATTCATTATTAGTACCAAACATTTCAACCTGTTGT
AGACATATACCCTTTTATGAGTTTGGGGTGGTGGTTGTTGTTGTT
GTTCTTCTTCTTCTTTTAAATATAGAAATCTATTATTTTTACCTT
TTTCTCAAAGCAAGATTCCCATACTAACTATGTACTTCAATCCAT
ATCA

In some embodiments, the intronic domain that promotes trans-splicing primarily in the brain comprises or consists of sequences from the gene CABP1. In some embodiments, the sequences from gene CABP1 comprise or consist of:

(SEQ ID NO: 287)
GGGTCGTCCTAGTCCCCCCGCACGGTATTGGGAGTGGAGGCAGCC
CCACACGGAGGCGCTGGGAGACTTTCCTCCCAGCAGTGTCCTGTC
ACTGCATTCCGGCCCCTGCGTTTGTAGGCAGTGATGTCACAGAGT
GCCTTCATGCTCCTCGGGTCTCCGGTTCTCCCCGGACCTCTGTAG
TCCTCATTGCCAAAGTTGTACCCCCTGGGGAGTGCACCCTGCCTG
CATTTCTGGAGGTTCGGGCGGTTCCGGGGCTCTGCAGGAGCCTCC
AGGCGTGATAAAGAGCGCTGCCGGCACTCCCGGGGTCTCGGCCTC
TTTGGGGCCCATTGCCAAGCCCCTTCCCTCCTCGTGCTCCCGCTC
CGCGTTGCAGCGTCCCCTTCGCCGCCAGGGGCCGCCCGGAAGCGG
GGCTCCCCCAGGGCGGGGCGGGGCAAGGGGCGGGGCGCGGCGGTA
CCTGGGCCGCCAGGGAGCCGGGGGTGGGCTCTCCGGGGACAGCCG
GCAG

In some embodiments, the intronic domain that promotes trans-splicing primarily in the brain comprises or consists of sequences from the gene CABP1. In some embodiments, the sequences from gene CABP1 comprise or consist of:

(SEQ ID NO: 288)
TAAGGGCCGCGCCTCCCGTCAGCGCTCCCGGGAAAGGCGCTCGGG
ACCCTGCCGGCCGCGGTTGCGCGTCCACAGCCTCCTCCCGCGGCC
CGTGGTCCCCCACGGATCACGCCTCGGCTCACCTCGTCCTCCCCG
GGCCGGCGCCCTTGCCGGCACTCCTCCTCCCCGTGCCTGATTCAG
CACCCCGTGCGCTGTCCACGCTCCGGGCCTCTTGGGGCAAGGCCC
TCCCCGCTAGCCTCCCTCATACCCGCCAGAACCCGCCAGTCCTGG
AAGAGAGCGACCTCTACGACCGTCCTGGAGATTTGCGGGGGTCAA
GGAGGGGCGCTCCGAGCAGGTGGCGCCAAACCCACTCCTCTGGCC
TCTGGCCAGTACCGCCAGGGAGGGACTCAGGAGAGCAAGCGGTTT
GGAGTCTGGGGGTCAAGAGAGGGGACCTCAGAGAACACAGAATTA
CTTGAGGGCGAGGGACACTGGCCACTGCCTATCATGTCCCTGAAG
GCCT

In some embodiments, the intronic domain that promotes trans-splicing primarily in the brain comprises or consists of sequences from the gene CDK2AP1. In some embodiments, the sequences from gene CDK2AP1 comprise or consist of:

(SEQ ID NO: 289)
GTGAGCGCGGAGTGAACGAAGTTGGCGCAGCTCTGGGGCTGCTGG
GGCCCGCGCGCGGGCGTGGGGGCAGTGGGTCACGTGGACGCCCCA
TCTGGGAGCCCCCAAGGGCTGGCCCTGGAAGGCCCTCGATGCAGA
AGCCGGGAGGGGCTCCCCTCAAGCTTCCTGGGGCTGCGGCCGCAG
CCGGGCGCTCCCAGGAAACTCCTCCAGAAGTTGTCAGGCAGCTTC
GGGCTGAGCCCAGGGGACCGAGGGAGCCATGGGGAGTGGGCACAA
CATTGAGGGATTCGTAGGTGCCGGGCTTTGTGACCTCACTGGGGC
CCTTCTTCTCTGACACTTGGTCGGAAACAGACTTCCCAAAAGGCG
GTTGTGTTGCGGGGCGGGATGGGGGGAAGGACTCGACGGGGAGAG
GAGTGGCCCCGTTGGTCACCGGTGCCACGCTGGCTCTCCTGCTCG
AGTTTTGGGCCTTGTTTGCAAACGTTGACCTCGTAACCCCCCAAA
TCGG

In some embodiments, the intronic domain that promotes trans-splicing primarily in the brain comprises or consists of sequences from the gene CDK2AP1. In some embodiments, the sequences from gene CDK2AP1 comprise or consist of:

(SEQ ID NO: 290)
ACTTGGCCGCGCACATGCCCGCCGCCGCCCTCAACGCCGGTGAGT
GAGCCGCGTGCGACGCGCCCCAAGCGCCGCCAAGTTGGGGGTCCG
CGCGCCCGGCCCCGGCCCGGGGATGCCCGGGGCGCCCGCGGAGGC
CGGGGGCGGGGGGCTCAGGGTCCCGGCCCGGGGCAGCGCTGCCGC
CGCCATGATTCCCGAGCCCGCGCCGCGAAAGTTCTCTGGTGCGCC
CCGCACGCCGAGGGCCTCGGCTGGGGGTGCGGGGCGGGCGGCCCG
GGGTGACCCCCGCGCCGGCGGCTGCGGATCGCAGGGAGCGCGGCG
GGCGAGGGCGAGGGCGGGGGAGGCTTGTTCTCGGCTCCTTTTTCC
TCCGGCTTCTGGGCGGCTCGGCGAGGGCCCCTGCCGGCGGGAGTC
GGGAGCGGCCCGGGCCGCGCCGGGAGCGGGGCGCGGGGCCCCGCC
GCCCTGGGGAGCGGGGTCGTGAAGCAGCCCGGGCGGGGGGCGCTG
CGGG

In some embodiments, the intronic domain that promotes trans-splicing primarily in the brain comprises or consists of sequences from the gene COMMD6. In some embodiments, the sequences from gene COMMD6 comprise or consist of:

(SEQ ID NO: 291)
GTGAGTTTGACTTTTTTAGATTCCACATATAAGTGAGATCACGCA
GTATTAGCCTTTCTGTGTCTGGCTTATTTCACTTTGCACAATGTC
CTCCAGGTTCATCCATGTTGTTGCAAATGGCAGGATTTCCTTCTT
TTTTATGGCTGTGTGATATTCCATTGTGTGTGTATGTATACACAT
ACATAGTATATACATATAGACATACATTATAATCATATTAAATAT
AAATATACACAAAAACATTTTCATTAGAGGCAACTCTTTTAAAGA
GAGTTTTTACATGGCTTAAAACTAACCACGAGATAGCCTGTTAAT
CACATTACATGACTGAGAGCTAAGGAACATTCTTTGAGCTTTTAA
TTTATATCTTTTTGATAACTAAAATTTTGAACATCAGTACATATT
TTTAAAGAAATAGTTACACTTTGCTTCATTCTGTGACAAATTTGC
ATATAAATTAAAACAATACAAATCCTCTTAAGTAGCAAAATAGAA
AATT

In some embodiments, the intronic domain that promotes trans-splicing primarily in the brain comprises or consists of sequences from the gene COMMD6. In some embodiments, the sequences from gene COMMD6 comprise or consist of:

(SEQ ID NO: 292)
AAAGAGAATACACGTTTTGGTCTTTTTTGTAATTCACAGTGCTGG
CCTAATTGTTATTATTCTGTGCAGCATTTGTGCTGTCCTGATTCC
ACCTCTAGAAATTTTTTCCTATAGGAAAAAAAAAATCACAATTGG
ATTAACTTTTTTTCCCCAGCTTTATTGAGGTACAGTTGACAAAAT
TGTGTATTTTGAAGGTTTCACAATGTGATGATATGATAAAAGATA
TACATTGTGTAAAGATTACCACAGTCACTCTAATTAACACATCCA
CCCCCACACAGTTACCTTTTTTTTTTTTGGAGAGAGTGTGTGCAT
GTATGTGTGTGTGTGTGTGTGTGTGTGTGTGTGTGCTGAGGGCAT
GTAAAATGTTTTTTAGCAAATTTCAAGTAAACAATAACAATATTA
CTGTAGTCACCATATTGTACATTAGATCTCAGACTTACTCATTTT
GTAACCAAAAGTTTGTACCTTTTTACTAACATTTCCCCATTGACC
CCCA

In some embodiments, the intronic domain that promotes trans-splicing primarily in the brain comprises or consists of sequences from the gene SLAIN1. In some embodiments, the sequences from gene SLAIN1 comprise or consist of:

(SEQ ID NO: 293)
GGTCATCACTGAGCCTCTTGAATCATTTCACATCATTTTATATAC
CAAAAATCCACTGAGATAATTGGTTGAAAAAGATAAAATTGCTCT
TTTTTACAAGTTCTTTTTGTTTGTTTTATTCTTTATAAAAGTGCT
TCCTGTTACAATTCTTTAGCTATGTACTGAAGAACAACTAGGATG
TGATAGCTTCCACTCCTGTCCTCCTCCTCCTCCACTGTAACCTTG
GGCACTAGTGGAGCATTATTGTTTTGTTAGCCTGCCTTTACAATA
ATTTTCCTCCCCTTTCTGAAAAGCTTCAATGCTAAACAACAATGT
AGAGCATTCTTTCTTGCAATAGACGCTAATTGTGTTCCTAGTTGG
GATCTTGCTGTCATAGCCACAATAACTGTGTCAATTGTGCAGGGA
GGCTGGGTCACAGTGGTTATTGTGCAGAGTGATGAATGCTGCTTA
CTTGTGATGTGCTGCCGCCCGCATCTGTGGGTGAGAACACGCAGT
TACT

In some embodiments, the intronic domain that promotes trans-splicing primarily in the brain comprises or consists of sequences from the gene SLAIN1. In some embodiments, the sequences from gene SLAIN1 comprise or consist of:

(SEQ ID NO: 294)
TAAGTTACGGGTTGGATAAGTGACATATACGACTGGTGTCAATTG
GCAACCAGGCTTCACAGTTTCATGAAATGTGTGGATTCTGAAAGT
GTTGTCATTGCGAATGTGGTTTCCTTGCCTAGTTCTTCCACATAC
TTAAGGAAAAGATATTTTTAATGTAATTGTTAGAATGTGTTGGTA
CGTAATGATTCTGAAGTAAAAGGAATTTTTTTTTTTTTTTTTTTA
AGTAAACTTGGTGTTTTGCAAGGATCTGAAGAATCCCAGTTTGGA
GCTGATTATTTTCTTGACTGTCCACATCCCACAGTTTTTTTTTTT
TCTCTCCATCCCACTGCCCATTTTAAAGCACTTTTCAGCTGTCTG
ATTGCTCATTAGCATTGCTCATTAAAAGTGGTCTGGGGGCATGCA
TTTCAAAGCAGTCCTTTTAATACTTGGTTTTTCAAGTTTTGTCAA
ATTCCCAGTGAATACAAACCAAGTTATTTCTTGAAGTTTAGCCTT
GTTT

In some embodiments, the intronic domain that promotes trans-splicing primarily in the brain comprises or consists of sequences from the gene FARP1. In some embodiments, the sequences from gene FARP1 comprise or consist of:

(SEQ ID NO: 295)
ATCTTATTTCTAGATTTTTTTTTTTCCTAAGCAATCTCTTGAAAA
GGAATATCAGTGAGCAGATTTCTCTGGGGCCATTTGAACTTCATT
AAAATTTCAGAGAGGGCTTTTCAATGAGAATATATTCATGTAAGC
ACTGGAGAGTTTTGCATAATTTTAAAGTTGAACAGAATTGGGTAC
AATTAGTGATGAAAATACAGTCCATCCCAGATTCCTAGCTGATTG
CCATTTTGGTTTTCCCAGCAAACACCCTTTCTACCCCACTTCCTA
GCTTTATTGAGATATATCTGGTATATAAAAAATTGTACATAACTA
ATGTATACAATTTGGTGAGTTTGGATATAAGTATAGGCTGGTGTT
ACCATCACCCAGTCAAGGTAATAAACATATCCATCACTTCCAAAA
CTTTGCTGTGTCTCTTTATTGTTACTGTTTTTTAAAAAACAACAA
CAAAAAGCGCCTTTTCAAGGGAGTAGCATTTGCTCTCCTCTCTTC
CCAG

In some embodiments, the intronic domain that promotes trans-splicing primarily in the brain comprises or consists of sequences from the gene FARP1. In some embodiments, the sequences from gene FARP1 comprise or consist of:

(SEQ ID NO: 296)
ACATGTTTGTCTTACCAGCTTATTGATAAGCACTTTGTGGGATGT
TTATGTGAGAACAGTTGATTTTAATGAGAAAACAGATTTTCAGCT
CCCATGTTCAGTGTTTTATGTGACAAAATCATACACTGCAGCAAC
TAAGTAATACGAAGTGAGTACTTTAATTGAAACAATTAGAGTGAC
ACAGGAGGCATTTTCTACTTGTGTAAACGCAAAGCTTAATTTTTA
AAAAGCCTCCCCCAGTTTTCCTTCCTAAAGGAAGTGGATGATCAA
GCCTGAGCTAGATAGAATAGCAGATGTGTATTTCATTATTAATAC
AAGAGGAATCTTTTTGGGTTAATAGAACTATTCTTTAGAAGAAGA
ACTTGTTTTGTGTTACAAAGGTGTTACATGGTTACTTACAGTAAC
AATTTCGAGCAGCACAGAAAATGCAGAAATGTATCCCATTCTTAG
GATTTTTGAGATAAATTCTTTTTTTTATTTGGAGAGGGAGTCTCA
CTCT

In some embodiments, the intronic domain that promotes trans-splicing primarily in the brain comprises or consists of sequences from the gene STK24. In some embodiments, the sequences from gene STK24 comprise or consist of:

(SEQ ID NO: 297)
GTAAGGGAGAGGGCATGAAAGCAGCGAGGATAGTAGCTGCTGAAA
ACGGGGTATGTTTCATTATAGCTAAAAAAGGATGCGCTGCTTCCC
CCGCATCCCCGAAAGCTGCTCATCCTAGCAGCTTGTGTTAGAGGA
GGTGGAGGTGCAGGTTCAAGGCCGCGTGTAGGGGGAGTCAGGTGG
GAGGTACCCACCCGGGCATTCTGTGGCATTCCCAAAAGTGACTGG
TGATACTCAGCCTGGGGTATTGATAAGCTTGAAAATAGGAAGAGG
AGGATGTTGTAGGGCTGCTCACGAGCTCCAAAAACAGTCTTCACT
TGAAAAAATTATCCTGGGCTTTGATTGTGGGTCATTGACTATCTT
GGTAAACCCAGGAGTCTTGGACTGTGTTTTTTAAAACCTGTAAAC
ACACAGCCAGGCTTCCTCCTAAGTATGACCGTCATAAGACATTTG
AAATGTAGTGCCGTGACTGAAAACAATTGAGCAATTGTTCAGGAA
AAAA

In some embodiments, the intronic domain that promotes trans-splicing primarily in the brain comprises or consists of sequences from the gene STK24. In some embodiments, the sequences from gene STK24 comprise or consist of:

(SEQ ID NO: 298)
TTCCGTGCACACATGTTGAGCTAGAGATGTCTGGGTAGAAAGAGA
GAATGCTTGGGAAGCAGCTGGTCTGGGGGGTTCCTGTTCAGAAGG
AAGGCCTGAGCTAGCGCTTTGAAAGCTGTCATGTACGTGAGTCTT
TAACACAGCCTTTCCTTCAAGGTGGCAATTGATCAGAATCCATTG
GGGTGTTTTAAAAACATGGATTATTCACCTCCTCTTCTGAACTCC
CATATATTTCTGCAAGAATGTCAGATGTCCTAGGTGGTTCTGTTC
GGGCAGGTTTGGGAAACAAGGGCCCCAGACCCTGAAACTACCGGG
CACCCCTGGGGAGAAGGAGTAGAGGCCCAGGCTGGGACCTAGAGT
AAGGGCCAGCCAAGGTGGAGTCTGCGTGAAGATCCTTCTGGAACT
AGAGGGACTGGAAGAAGCCAGCGGGAGTGGGGCTGTGGATGCTAA
AGGACTGGAAAGTTACCCAGAGGAGGGGGAGGGCGTGGTTGACAA
GGCT

In some embodiments, the intronic domain that promotes trans-splicing primarily in the brain comprises or consists of sequences from the gene RNASE1. In some embodiments, the sequences from gene RNASE1 comprise or consist of:

(SEQ ID NO: 299)
GTCACTTTGTAGGTCACCACCTAGAGGGGAGGAAGACCTCGCTTT
GGAGAGTGGGAATAAAACGCTCGTGGAAAAGGGTACACGTGAGTG
TCTCATTACACGAAAAACCTCAGGAAGTAGGATGGACGTCTTTCT
GGCTGACCTGAAACAGAAAGTGTAAACCAGGCAAGCCATAGGTGG
GAGCTGGAGTTCTTTTTCTAAGAGGGCTCTTTTCCTTCTCTCTCC
TTTTCTCAATTAGGCTTTTCTGGGAAAGTGAGGCCACCATGGCTC
TGGAGAAGTCTCTTGTCCGGCTCCTTCTGCTTGTCCTGATACTGC
TGGTGCTGGGCTGGGTCCAGCCTTCCCTGGGCAAGGAATCCCGGG
CCAAGAAATTCCAGCGGCAGCATATGGACTCAGACAGTTCCCCCA
GCAGCAGCTCCACCTACTGTAACCAAATGATGAGGCGCCGGAATA
TGACACAGGGGCGGTGCAAACCAGTGAACACCTTTGTGCACGAGC
CCCT

In some embodiments, the intronic domain that promotes trans-splicing primarily in the brain comprises or consists of sequences from the gene RNASE1. In some embodiments, the sequences from gene RNASE1 comprise or consist of:

(SEQ ID NO: 300)
AAGCCAAACCCCTTTGTAATCTGTATAAGGTCCACACCCCGGGAG
CTGAGTGATTGCAGAAACTGGCCTTCCATCTCTCTCAGACACCAA
GCTGCAGATCCAGGTAAGCAGCCCAAGAGGTGTTCTCAGGTGCCG
CTTCTGCTTCAGGGTTGCAGAAGAGGGAGCTAGGGCCAGTCCACA
GCACTGAGAAAGATTGGGTGATGCTAGCGCTGAAAAAATGTGGCC
CAGGCAAACATCAGCTGGGGGCAGAGGGGTCAGGGTGGAACGTGC
AGGCTGCAGAGCATCCATGGGACTGGAGAAGAAGGAGCCTCCAAA
TTGGGGGAGGCCAGCGTTTCTCTCTGGAATACAATGGAATCTTTT
ATGATGCCAGTGTCAGGCACTGGCCTGAGTTCCCTTATGCTCCAA
GACAACAAGGCTCTGTCACCCATGGCCCTGGATGTCGGGGATACA
ATTTGGTTCATGATTTATTCAAACTGATGTTTCGATGAGCCATTT
GAGC

In some embodiments, the intronic domain that promotes trans-splicing primarily in the brain comprises or consists of sequences from the gene NDRG2. In some embodiments, the sequences from gene NDRG2 comprise or consist of:

(SEQ ID NO: 301)
GTCGTGGATGGGGCGGTGCTGAGGGCAGCCGAGGGATCCCTCTCA
GGGCTGAGGGATCCCCGCACTCCCCACACCGCCCCAATGCGGGGA
GGGGGTGGTGGAATGTTTTGGTGAGCCGAGCAGAGCCTGGAAGCG
TCCAGCCGCCCGCCCCTCGCCCCCCGGCCCCAGCACAACCCTTTC
CTGAGCGGCAGTCTGGGCGGGGTGAAAGGGGGGGCGTGTTTACTG
GGGGTCTGGGGCGGGTCTGCGGGGAGCCTAGCGCCCGTGGGCGGG
CTGGGGCGGGCCGGGGGCGGGTCTGCGGGAAGCCCGAGCCGGGGC
GGGCCCGAGTCAAAGGCAAGTGAAGGTGGAAGCGGCCGCGGCGGC
AGCAGGTAGGGGGAGGGGCAGGCGAGGGGCCGTAGGGCCTGGAAG
GCGCCAGCCGGGCCGGCGGGCGGTGTGATTGATCCGCGTCCCCTG
GAGCTGGAGGCTCGGGGGAAAGGGCCAGCACGGAGCGGGCGCTCG
GTTG

In some embodiments, the intronic domain that promotes trans-splicing primarily in the brain comprises or consists of sequences from the gene NDRG2. In some embodiments, the sequences from gene NDRG2 comprise or consist of:

(SEQ ID NO: 302)
GTGTGTGTGTAAAGTCTACAGTGGCAAACCCACTCGGGCACCGAG
AGGGACGCGGCAGACCCTGAGACCATCCCCCGGGCATTGCGATTC
AGCGGGCCTGAGCTGGTCTCCTATTCTGGGCCGGACTGGGAGGGG
TTAGCGGCGAAGCCACAGGGTCTCTTGGGTCCCAGCCCAGGACAC
TGCGTCCTCCCCAAGCCCCTACTCCACCCCCGCAGGGTTAACTCC
GCCCCTGCGCCTGTTTACCCAGGAGTCAGAGCCTCTGGGGGTCCC
CGGCCCCTCCCCCGCGCCCCTTGAGGCATTGACCCCAGAGTCCCT
GTTGGCCATCCTCTCCCCACCCCCCGCCTGCCCGCGATCGCCCTC
TATTTATAGCGGCCCCCCGCATTCCCCGCCCCCACCTCCCCGCCT
TGTTGTCCAACTTCTCCCGGAGCAGCCGGAGAGCAGGCGTCGGGA
CGCAGCAAAGAGAGGAGAGGTACCAGGACTCTGGGCACGAGGGAG
TCGG

In some embodiments, the intronic domain that promotes trans-splicing primarily in the brain comprises or consists of sequences from the gene PSME1. In some embodiments, the sequences from gene PSME1 comprise or consist of:

(SEQ ID NO: 303)
CAGATACCTCGGATTGAGGATGGTAACAATTTTGGAGTGGCTGTC
CAGGTGAGAGCGCTGCCCCACTTCCCTGCTCTTTTCTAGTCCATG
CTTCCTTCCACTTTCCCCCTTGCTTTTTTTCCCTAGGAGAAGGTG
TTTGAGCTGATGACCAGCCTCCACACCAAGCTAGAAGGCTTCCAC
ACTCAAATCTCTAAGTGAGTGACCACCCATGTGCACACTGTTTTT
GTTTTGGGAGACCTCCTTCTTCTACTCCATACACACTTCTCCTTC
CACAGGTATTTCTCTGAGCGTGGTGATGCAGTGACTAAAGCAGCC
AAGCAGCCCCATGTGGTAGGTGAGGCCCAGGTCAGGGTGCATGGG
GGAAGGACACATGTAAGGTCAGGCCTGACCCGAGCTTCCCACAGG
GTGATTATCGGCAGCTGGTGCACGAGCTGGATGAGGCAGAGTACC
GGGACATCCGGCTGATGGTCATGGAGATCCGCAATGCTTATGTGA
GGAG

In some embodiments, the intronic domain that promotes trans-splicing primarily in the brain comprises or consists of sequences from the gene PSME1. In some embodiments, the sequences from gene PSME1 comprise or consist of:

(SEQ ID NO: 304)
CTGTGTTATATGACATCATCCTGAAGAACTTCGAGAAGCTCAAGA
AGCCCAGGGGAGAAACAAAGGGAATGATCTATTGAGAGCCCTCTC
TCCCATTCTGTGATGAGTACAGCAGAGACCTTCCTGCTTTTTACT
GGGGACTCCAGATTTTCCCCAAACTTGCTTCTGTTGAGATTTTTC
CCTCACCTTGCCTCTCAGGCACAATAAATATAGTTATACCACTGC
CCATCAGCCCAAGTCTCTTTATTGGAACCGGGCCCCGCTGGCCCT
GGCTCAGGCATTTCCATTTCCATTAGTGGGACCAACCCACTGGGT
GATTTGAGTGTTGAGCCGCTGGTTGGTCCAGTCCTGCCGGATGTC
ATCAAGGTGGCAGTCAAAGTCCACAAGGTGCTGGTGGGCCCGATC
TTCCAGTAGAGCTCCCACCATCTGCCGTGACTCTTCCCAGTCCCT
CCACATCACTCTGAAATAGTAACCCCCATGGAGGTCAAACAGCAA
GTAG

In some embodiments, the intronic domain that promotes trans-splicing primarily in the brain comprises or consists of sequences from the gene REC8. In some embodiments, the sequences from gene REC8 comprise or consist of:

(SEQ ID NO: 305)
CCAGAGTCCTGCCTTTTCTGTCTCCATTTCCCTATTCTCTGTCCC
TGGCACTTGAGCACCCAGTGGCTTCCTTGAACCTGGCCCTGTGGC
ATGCCTCTGGGATCCACTCTCCTGGATCCACTTGCCTTTTTCAGA
AATATTATTGAATCCCCTCCCCTTGCTCTTCCTCTCTGGACAGGG
TGAACGGGAGCTCCCAGAGGTCAGCCGCCGAGAACTGGACCTGCT
GATCGCAGAGGAAGAAGCTATCTTGTTAGAAAGTAGGTGTCTCCG
GCAGCGTAGGGCCCGCCTGGAGCTGGATAGTCAGACCCCTGGCGT
GGGCATTCTGGGACTGGGCAATGCTCCCATTTCTCTTTTTCTGTC
CTCTGAACTCTGATTCTCTCTCCACAGTCCCGCGGCTCCCACCTC
CAGCTCCTGCAGAGTAAGGGCAAGAACTCCTAGACCAGGTAGGGT
GTCAGTGCTGGGAAGGGTCTCCTCATTTCTCTTGCCCATTTCCCC
TCAG

In some embodiments, the intronic domain that promotes trans-splicing primarily in the brain comprises or consists of sequences from the gene REC8. In some embodiments, the sequences from gene REC8 comprise or consist of:

(SEQ ID NO: 306)
TGAGTGCCCACCATGCCCCAGGGGCTTTTCTGGGAGTACCTGGAT
ACTGCTGCAGACAAGGGCTTTATATCCCAACTTGCTAAAGGGAGG
ACCCTGCAGTTTCTTGCCCTGGCATCTGCAAGGGGTAAGGGGCTT
ATGGGACAGAGCCCCTTGGGTGTTGTTGCAGAGGTGACCCCCCCG
GAGGAGCTGCGTCTGCCAGCCCCACCCAGCCCAGAGGTGAGTAGC
CTCCCTTCTAATCCTCCTCCTCCTCCTCTTTGCCCTCCCCCACAA
GGACTGCCTCCCCAAGCCCAGGGCCACTGCTAGCTTACAGGGGTC
CTTAATGCAGATGATAAAAGATGCCCCTTTCTACGCCCCATCGTA
TCTGGCCTACGCTTCCCAGATGGGTACCCTTATGCAAGGTATGAG
CTGCTCAAGTGCATGGAGACCCCGCACAAGCCCTCTCCAGGTTCC
TCTAGGGGCGGGTGTGGGGTCCTGTTAGCAGTCCACCAGAATGAC
AGGA

In some embodiments, the intronic domain that promotes trans-splicing primarily in the brain comprises or consists of sequences from the gene SDR39U1. In some embodiments, the sequences from gene SDR39U1 comprise or consist of:

(SEQ ID NO: 307)
GTGGCGGGACAGGCTTCATTGGGACAGCCCTAACCCAGCTGCTGA
ATGCCAGAGGCCACGAAGTGACGTTGGTCTCCCGAAAGCCCGGGC
CCGGCCGGATCACGTGGGTAAGTCCATCCTCTGGAAGCGGGTGGG
AGGGCAGAGTTGGGCGGCGCAGGGCGGGGCAGGGGCACTGTGTGC
TTTCTCCGACAGGATGAGCTCGCTGCATCGGGGCTGCCGAGCTGC
GATGCCGCCGTCAACCTGGCCGGAGAGAACATCCTCAACCCTCTC
CGAAGGTCAGCCCGGGCCCTAAAGCTGATACCCACTAGAGCACAG
GGAGGACAGTGCCCCACTGATGAGAACCTGTGAGCTATGGGTAAT
GGAGCTCCCAGATTCCGAACTAAACCGATATCCCAGACTACTCAT
TCTGCTCCACTCCTCCACCCCCACCTGCTCCTCCACCCAGTCAGC
CAATTGGCTTAAGTCCTCACGTATAACTCAGGATGCCCAGGAAAT
GAAT

In some embodiments, the intronic domain that promotes trans-splicing primarily in the brain comprises or consists of sequences from the gene SDR39U1. In some embodiments, the sequences from gene SDR39U1 comprise or consist of:

(SEQ ID NO: 308)
CTTGCTATTGAAACACCCAGCCTATATTTACAAGTTTTTCCGGGT
CGCCGAGAAGGGTCACGCACAGGATTGCGACCTCGATTGGAACGC
GCTTCCCGAAAGGGGCATGCGCAGAGAGGTCGTAGTCGCGTCTGA
GGTGAGGTAAACTCTAAACTTTACGCAGGCGCGTTAGGTCCTAGT
CGCTATGCGTGTGCTTGTGGGTGAGGGAGGGCAGAAAGGGAGAGT
GCTGGGCGGGCTTAGTCGGAGATTGAGGACTGGGAATCCGCTTCC
GGGAGGGCACTGTCTAGTGCACAGGCAACCTGGCCTTGGCCTCCT
AGCCCGAGAAGCCGAATCTCCCTAATCCCTGTGACCTGTGTCACC
TCTGCATCGCGAGGAGGGGGATAAGTGGGGAGAAGCCTGGTGTCA
GATGGGATGGCGCCGGAAGAGGGTGCCACAGCGGGGACGGAAGGC
GCCCCCACCCCAACTCCACGGGAATATAAACAATTTGTATTTTCC
GATC

In some embodiments, the intronic domain that promotes trans-splicing primarily in the brain comprises or consists of sequences from the gene RTN1. In some embodiments, the sequences from gene RTN1 comprise or consist of:

(SEQ ID NO: 309)
GTATAGTTGCTTAAGCTGTTCACTTCACAGTCTTGACTGTTATTT
TCCCTAAAGGTGTAATACTGTAAACTGCTACATCAAGAAGAATAA
AAAGCAAGTGCTAATTATATTCTAGAGTCAGAAGACCTCCAGTTG
TATGGCACCATGAACCAGTCTAATCCCTTCATTGTACCAATGATG
AAAATGTGGCTGAGACATTTGTCCAGGGTTGCCCAGTGAGTCACT
GGCAGAGCTGGAATCAAACCCAGGTCACATAGCTTCTAGCTCTAT
AGTCTTTACAACACACCAGATTGTCTCCTTAAACCATTTTTAGTT
CCTCCGACAATTTCCCAAAATTAATATTAAGATAAAAAATCTAGA
TAGGGAATGTGAAAACCATATATACTTTTGCATTGAAGGAAACTC
TAGATTCTACTAATATTAATAGTAAGCCCTCCAAGTGTGCTTATG
TAGTTACAGCTCTGAGGCTAGAGGTATGCCCAGCCTCTCACTTGA
TGAG

In some embodiments, the intronic domain that promotes trans-splicing primarily in the brain comprises or consists of sequences from the gene RTN1. In some embodiments, the sequences from gene RTN1 comprise or consist of:

(SEQ ID NO: 310)
TATGGCCCAGGTCACTTGAGTTTTCAATGCTCTTTCTATAAACCT
GGAAGTAAATTCTAGAATTTAAACATGAATTCTTAGAGATCTGAT
GAGGAACAGTTGGCAGCTAGGATTTTGAGCTATCTCAACTACTTA
AAAAAAGATAATTTATCTCCATGAAGTCATGTGTGGAGAATTCAG
CAAATGAGCTGATTTGGCACTAATTTTTTTTCTCTGGTCCCAGCT
GGTTTCTCTGAATTTTCAGCCCACCCATGCTGTGTCTTAGGTAGC
CAGAAACAGTCACAATGCTCCACTGAGAACCATGCTTTCTGTGTT
CTCAGTGCATGGAAGCAGGTGTGTACGGTGCCCTGTGGCCCTCAC
TGGCTGGCATAACCCCCCAGAGATGACTCCTGCTCTCACCCTCAG
AGTGAGAGTAGGAACGCTTAGCTGTTACTGGGGTTCGGCAGCCTG
TCTCATGATTCCCTCTGACGGAATTTTCTGTGGGAATGGTTTGTT
GACA

In some embodiments, the intronic domain that promotes trans-splicing primarily in the brain comprises or consists of sequences from the gene SLIRP. In some embodiments, the sequences from gene SLIRP comprise or consist of:

(SEQ ID NO: 311)
ATAGATATTATAAAAAGTAAATCAGTGAAGTACAAGCACTGTAGT
TCAGAAGAGAAACAGATCGTGTAGTTTTTAGTCTTTAAGATTATA
TACTCTAAATCTTTGAAGTCTCACAGGGCTGGTACATTGTCTTGT
ACTGTGATAGGAGCCCAATAAAGTAATACTCAGTGGATGTACTTG
GTTTGGGTCTTGAAGGATAGTTATAGTGTTACAAGTTAGCCAGGG
GGAGATAGGATTACCAACATATGGAGGGGAGAGAACAACATATGC
AAAGAGGGTAACCCGCAGGACAGATAAAGATAATTCAATTAGTTG
TACTAGAGAGTAACTAGGGGCTAGCAGTGGGAAATAAGGCAGGAA
GGGTAGGTGAGGGCAACTCAACACATTTTACTAAATTGTGAAAGA
CCAGTAAGTTGGGATTTTATTTGGAAATCTTCAGTATACCGGGAA
ACACACTGGATTGTAATTTGTCTTTTTCTTCATTGTGATCTGCTC
ACAG

In some embodiments, the intronic domain that promotes trans-splicing primarily in the brain comprises or consists of sequences from the gene SLIRP. In some embodiments, the sequences from gene SLIRP comprise or consist of:

(SEQ ID NO: 312)
TACATTATGGCTTTATAGTCAGTACCATAGCTGCTTCCTAAATCT
TAAGCCTTTAAGGAAATGCAAAGTAGTTCCTGTCCACAAGAAATC
TGGTGACCCTGTCTTTCTACAGTCTAAGATAGAGAATCCAAAATA
TAGTAACTACTTTTAATGTTTTCTTCTGCCACACAGGTCAGCTGA
AAGAACACTTTGCACAGTTCGGCCATGTCAGAAGGTGCATTTTAC
CTTTTGTAAGTATTAAGGAAAAGTAGGTGGGAGGTGGGGATGGCT
AATGGGTACAAAAAAAATAGAATGAATAAGACCTATTTGATAGTA
CAACAGGGTGACTATGGTCAATAATAATTGTACATTTTAAAATAA
CTCAGAGTGTAATTGGATTATTTGTAACTCAAAGGATAAATGCCT
GAGGGGATGGATACCCCATAAGTCAAAGGATAAATGCTTGAGGGG
ATAGATACCCCATGCTCCATGATGTGCTTAATTTACATTGCATGC
CTAT

In some embodiments, the intronic domain that promotes trans-splicing primarily in the brain comprises or consists of sequences from the gene HSP90AA1. In some embodiments, the sequences from gene HSP90AA1 comprise or consist of:

(SEQ ID NO: 313)
GTAAGTGGACTAAGGTTTAAAATTACTATCCTAAACATTTCTAGC
TAATGTTAACCATCCATCCAACCAACCGGTGGTTGGAGTCACAGA
AAGCTAAAACAATGTCACTCTCTTTATTGTTTATTCCCTGCCTAC
CCATCCACTGCATTGACATCTACTGAGGGTCCACTACAGGCCCAG
GCAATGTCCAAGAGAGTCCCAGGTCCCTGCCTGGAGTGCATGTGA
CAATCATCATAATACAGGGCTATAGCGCATATATGACAAAGTGCT
GTGGGAGAAAAGAGAAAGACAATCCAATCCCTTGGGCTCCCAGAA
GAGGTGGCTTGTGAGCTGCGCCTTGAGCACCCACTCACGGAGGAC
AGCATAAGCAAAAGTGGGGAGGAATGTGGGCAGGAGGGGCGTGAA
GAAGCGGGCAGTGGGGTTTGGAGTGACTGAACCCTAGGAGGGTGG
TGGTGGCAAGAGATGATGGGGGAGTGTTGGAGGGGCCTGGGCAGG
CTAT

In some embodiments, the intronic domain that promotes trans-splicing primarily in the brain comprises or consists of sequences from the gene HSP90AA1. In some embodiments, the sequences from gene HSP90AA1 comprise or consist of:

(SEQ ID NO: 314)
TTGATGCATTTAGCTTACTTTCCTAAGGGGCTTTCAAGGGGGACA
CTTCCCCTGGATTCAATTTTCCCCTTAGCCACTAACTTTGAAACC
CAACCAGAATTCCAGACCCTTCAGTGCAGCTCTCCTGCAGTTGGT
TGAGTCATTAGGATAAGTCTGAATGGAGCCTGGAATAATAACAAC
TAAGTTCTAGTTTTTGCTGCAGGCCCTCTAGCCCAGTCCAAGAGC
ACCTGGCCACCCAGAGAAGCACTGCTGGAGTGGGGGATCAGGCCA
TACAGCATCCACAGAGAAGCCCAGCTCCTTACTGGGACCCTGGTC
CATTTCCCAAGTTGAACTTCCTGCAGGAAGAGAACATATCACATA
TTTTAATCCATTTGACAGTCACAGAGGAAGTCTAAGGAAGCCAGG
GCATGCCTGCTTGGGGCTGTCCAAGTTCATCCTCTGCCGCCCTAT
CTCTGTTATGTTTGGTTTGGAAGTCCAGAAGATTGTGTTTATGTT
CCCA

In some embodiments, the intronic domain that promotes trans-splicing primarily in the brain comprises or consists of sequences from the gene CRIP2. In some embodiments, the sequences from gene CRIP2 comprise or consist of:

(SEQ ID NO: 315)
GTCCCAGCTTCCTTGAGTGGAGTGGTTCTGGTCAGAGGGCTCTAA
GCTGGTGCAGCCTTGTGGTTGGGCTGGTGTCCTGGAGGCTGGCCC
TGGCCATGCCCAGAGGGTTGTACCAGGGCCACGGAGAGCCAGTAT
TCACAGCCCTCTACCTAGTAGGCCACCTGGGGGATCCCATCTCAC
TGCCAGGAGCAGCACCCTTGGGCCTCTCAGAGTGGGCTTCGCCCT
AGCCAGATGCCCCTCCCCGATATCTGCCAGACAAAGGGGGCTGGG
CCACTTGCTGAGAAAGGGAGCTGGGAGAGCCACTGGTCCTCGGCC
ACCTGAGGGGCTTGGCCAGATTCTGCCGGGCAGGGTGGGCCCAGC
CTGCATGCTCCCTCCCCCCAGGGCCTGCAGCTTGTCCCCTTCCCT
TCAGAATTCCGGCTCTGCTGGGCCCTTCCTGCCCCTCCCCACCTC
TTCCTGCCTGGCTCCCTAGGGAGGCCCCTGCTTCCATCCACTGGG
AAGC

In some embodiments, the intronic domain that promotes trans-splicing primarily in the brain comprises or consists of sequences from the gene CRIP2. In some embodiments, the sequences from gene CRIP2 comprise or consist of:

(SEQ ID NO: 316)
TGTGTGAGTGTAAGAGAAGTGTTTGAGGGAATGACAGGTGGGGGA
GATGCATATGTGTGGGGGGAGGCAGGTGTGTGGGGGGAAGCGGGT
GTGTGTCTGAGGAAGCGGGTGTGTGTGGGGGGAAGCGGGTGTGTG
GAGGCGGGCGTGTGGGGGAGGCGGGTGTGTGTGTGAGAGGAAGGT
GTGTGGGAGGAGGCAGATTTGTGGGGGGAGGCGGGCGTGTGGGGG
AGGCGGGTGTGAGGCGGGTGTCCGGGAGGCAGGTGTTGGAGCGCG
GGCAGGTGTTGGAGCGCGGGCAGGTGTTGGAGCGCGGGCAGGTGT
TGGAGCGCGGGCAGGTGTTGGAGCGCGGGCAGGCTCGAGCACAGG
CCCCTTCACCGCAGGCACTGAGGGGACTAACAGGGCAGCCCCGGG
CCCCTTCTCAAAGGCGGCTCTAGCGGGGTTCCAGGGCTGGGGGCG
CTGAGACCCAGAGGGAGAACAGGGCCTGGGGGCGACGGGCTCCTC
CGGG

In some embodiments, the intronic domain that promotes trans-splicing primarily in the brain comprises or consists of sequences from the gene APBA2. In some embodiments, the sequences from gene APBA2 comprise or consist of:

(SEQ ID NO: 317)
AGGGAGAGGATGGGATAAGCCTGAAGGGGAAGGTACCCAAGATAG
TCTTTGAGGGGTCATGGATTTAGATTGGGAAGAGTTATTGTCCCT
CCTCATTGGTCCTCATTATGTCTTCTTAATGAAAGCTGCTCATTT
TCTTCGTGAGAGCAAACAACCTGATCTTCTGACCATTTGTCTTGG
TCACCTGGGCTACCCAAACTGAGTGAGAAGTCAGGAATGAGCCGG
TAGGTTCCTGGGGAGAGGCGTGTGTCCTCGTGAGTTGCCGGTGCC
TCCCTTGACTGCTTTTCCCGGAGCTCTGTGTGCCCTCGTTCTCCT
TGGTGTGGATGCGTGTTGTTCTGATGATGAGTTAGCGGGGGTGGG
GATGGCGCACTGTTTGAACAGATGTGCCCTCACATGGCTGCGGGA
GGACGTGGTGGGAGGTGCTGTGGTGAAGTGGCCCCACACATGGCT
GGGCTTTGTGGGGTTTTGACTCTGTCCTTCCCAATGTTCCTCCCC
ACAG

In some embodiments, the intronic domain that promotes trans-splicing primarily in the brain comprises or consists of sequences from the gene APBA2. In some embodiments, the sequences from gene APBA2 comprise or consist of:

(SEQ ID NO: 318)
TAGGACCCTGGCTGTCCTGGGGAAGGGAGCAGAGGGGCCCGAGAG
CAAGGGACCTCAGGGTACAGGCCTTGCAGATGCTGAAGCGAGGCG
GTGGGGGGTGCTGGGTGCCTCACAGTTCTAATGGTGGCTGAGCTC
TTCATTGGTCCAGTTGGGAGACATGTTGCGTGGATGCTCCGGCCA
CTCTTAAGCTCACCGCTCAGACTCAGGACTAAAGCCGGTTGAGGG
CTGAGTGGCAACTCGTGTCTCGCAGAAGACACCCCTCCTCCCACC
CTAGAAGAGAGGTTCGATTTCTTGCCCACTGCCCTGGTCTGCTCA
TGGTGGGGCAGTATGTGTGGGAGAGCCCCTCAATCCTCAGGGGTG
TACTCAGACCAGCAGGGCAGTGGCAAAGACGTGGGGGTGCTGGTG
CTCACCCACTGCTGCGGCCAGTGTTGGGTGGCCTGGTGGCTGGGG
TCGGGGCCAGAGTGCATTTCACAATGGACGGGTCTCTGAAAGCCC
GGGG

In some embodiments, the intronic domain that promotes trans-splicing primarily in the brain comprises or consists of sequences from the gene SCG5. In some embodiments, the sequences from gene SCG5 comprise or consist of:

(SEQ ID NO: 319)
AATGTTTCCCTGCCACCACTCCCAACCTTCCTGAGCTTCTTGATT
TGTGTCTTCCTGACTCCCTCCTGTTAGACTTTTATTCCCAGAGAC
TGGGAAAACCCAAGATGCCACTATCTCTTTCTCAAAATGTAAGCA
AGTTTGGTTTATTTGTTTTAGTAAGAGAGGGAGAGAGTGATGAAG
TTAGGAGTAATGCAGAACTTTCAGGGAGCTACAAGGAGGATAAAA
ATTATGAGTGAGAACCGCCATTCCAGCCTAGCTTTCTCAAGAATG
CAAACAGAGGGAATTGGATACCTGCAACTGTTTTGTTTAATACCT
TTCTGCAATGATGCTCTGCCTAGCATGGAAACTTAAGACAAAAGC
AACCTCCTAAGGATTCTTTGTTACAACCCCTTTCTGGGTGGTCCT
CGAACCACAACCTGGAGTGGTTGCATCATTATAATTGTATTATCA
CAATTGCCGATTGTAGCCTATCAGTATACATTTGGTCTTTTATCT
GCAG

In some embodiments, the intronic domain that promotes trans-splicing primarily in the brain comprises or consists of sequences from the gene SCG5. In some embodiments, the sequences from gene SCG5 comprise or consist of:

(SEQ ID NO: 320)
TATTTACTGTGTTCTGATGGTTTGAAGTTTCCGTTAGCATTTTAA
ATAATATATTTGCACACTTCTCACAACAACCTTATAAGTAGATGC
CTTTATTATCCTATTTTTTTCAGAGGAGGAAGCTAATTTTTAAGA
GACTCTACTTTCTCATGGTTTTCCCTTTCTTCTTCTACACAGTGT
CTACATACTTACATATACACACCCACATGAGACTATAAGTCCTGT
GAGAGCAGGGACCTTATCTATCTAATTCATAGCTGTAGCCCTAGC
ACCTAGCACAATGTCTGGTACATAATAGGTGCTCAATTAATGTTT
GTTGAATGAATGAAGGAGCATCTAGTGTTATCACTAAAGAAAGAT
TAGAACCCAGACATGATGTTTTCTCCTCACTGCTGATCTTCAGGA
AATTGTGTCATCAGATAGCTGCCTTTTGAGTCAATGCCAGCCAGT
ACTTGTAAAAGAGCTTCAAAATGTAGAACCTACAGGATCTGACTT
GATT

In some embodiments, the intronic domain that promotes trans-splicing primarily in the brain comprises or consists of sequences from the gene SRP14. In some embodiments, the sequences from gene SRP14 comprise or consist of:

(SEQ ID NO: 321)
GTGAGAAACAAAGCTTTTTTTTTTTTTTTCCCCAAAAGGAGCCTA
CTGCCTTTCACCTCTTGGTGCAATTCTCATGTGGAAAAAGATGCA
GAGCTCCTTTTCTTGTTCTCTAGGCAGAGGAAAGTAGAGTAGAAA
AATAACCATTCGTGTATTATGAACCCTGAAGTTGGGGAGAAGTAT
TTTAAAGATAAGGATATTGTCTTCCGGAGGCTTTTGAGATACTAT
CTGGGGTGGAGAAGAGGATGCTTATATGGATAATAGGTATGACCT
CTTTTTTCCTTCTTTTTCAGGTGAGCTCCAAGGAAGTGAATAAGT
TTCAGATGGTGAGTTTTGGGTGTTCCTTATGTTTCCCTTCTCAGG
GCAAGAGTCAGTATGATAGTGAACACTGGGTAAGATTGATACTTG
GGTTCACTGCTCTCCGGAGATAAGTTTTACCAGTATGTTTCAAGT
AAAAGCTGCCAGGGTGTGAATTTCAATGCTAATACCGTCTTCCTT
TCTA

In some embodiments, the intronic domain that promotes trans-splicing primarily in the brain comprises or consists of sequences from the gene SRP14. In some embodiments, the sequences from gene SRP14 comprise or consist of:

(SEQ ID NO: 322)
CTAGTCTTTCAGTGGAGGTTGAATAAGGCAAAGGAAGAGGCTCCT
TGAATTGAGTGAATACTACTTTATTCTGCTGTGGCAGAAATCACA
TTAGTGTAGGATACTGACTGCATGAAAATGTAGACTATACTTAGT
TGGAAGTCTTGAGTTATCTTGAGAGGACTTCTACAGTAAAACAAG
CTTATGTGGTTTTACAGATTAAGTTAGATATCATGAAATAACAAA
AACCTAAGGACAAGTTATCTTGAGAGGACTTCTACAGTAAAACAA
GCTTATGTGGTTTTACAGATTAAGTTAGATATCATGAAATAACAA
AAACCTAAGGAGTGTTCTTCAGAGGTAGAATTTATTTTGTGGTAG
CTCGCATTGTGCTTAAATATGCTTGAATTAATGGAAGGCAGGTTT
AAAAATAAGATGGGGAGGGAGTAGTTTAAATATATCTTCTATTAA
TGTATAAAAAATTAATACTGAGAAGTCCTCTGACTTTTCTCAGAA
CCCA

In some embodiments, the intronic domain that promotes trans-splicing primarily in the brain comprises or consists of sequences from the gene SHF. In some embodiments, the sequences from gene SHF comprise or consist of:

(SEQ ID NO: 323)
GTGGGTCCTGGGGGGCTTCTGGCCAGGGTGACATGTCCTCACCCC
TCCAGGCCCTGTGGGTTGGGTTGGAGGAGCTGAGAGGTGGGGCAG
GATTTGGAGACCCCAGGAAGAATCTCATTTGGGAAGAAGGAAGAG
ACAAAATGCACGTTCCTCCCTTCCCCACAGCTGTGGGATGGTGTC
TTCTACAGGGAGGCCCCTTTCTAATGTGTTCCTCACCATGCTACA
AAAATCCACCATTTTAAATTCACTTTGGGGAGAGCTTTGCTTGAC
AAGCTCCTTCAGCAATGCAAATACTCCTGAGCTGGGGGTGGGACA
GACACAGCAAGTGTGGAACTCCTTGGCGCAGAGGAGCAGATCTGA
CCTTGCATCTGGTGACTGAAAGAGAGTAAGGGCTTCAAAGTGCTA
CAAGGAGAGAAAAAGAGCCCAAATGAGGAGAAAGAACGCACTTAA
TTCTTCCTCTATGTTCAGTTCTGCCCAGAATGGGCACCCAAGGGT
GTGT

In some embodiments, the intronic domain that promotes trans-splicing primarily in the brain comprises or consists of sequences from the gene SHF. In some embodiments, the sequences from gene SHF comprise or consist of:

(SEQ ID NO: 324)
CCTCAAGTCTGAGTAATCCTGACAACCTGAGGCGAGGCTGGCTCT
TTCTTGCTGCCTGCGAGTTCCCAGGCTGTGGGGAAAAGGAGAAAG
GGAGGCAAGGCTGTGGGTGCCAGGAGGCTATGGGAACCCAGATGT
GGTGTTGGGAGGTGCTGGTCACTCTTAGTCCTTCATTTCCTTTGT
TCCTGGTACCTTTGGGCCCTGGAGGCTTGTGAGAGGTCAAATGAA
GGGGACCCACGGGCTGACAGTGCAAGCCCCTGCATCCTGCTGCTT
GCCACCCAAGCCTCCCTCCCTCTGACCAGGCCAACCTGGTGTGGT
CAACGTTGGCTCTGCCCAGCCCTTGAGTGCCTCCCCAAAAGCTGA
CCTGCCTCTGCCTCTGCCTCTGCCCCTCCTCATTTCTAGGCTGTC
CTGTGTCCTGGCTCTGGGTTTTGGTGTCTCAACCTCTACCTCTTT
CTGTCTCTGTCACTGTCTGTCTCTCTTCTCACTTCTGCTGCTGGC
TCCA

In some embodiments, the intronic domain that promotes trans-splicing primarily in the brain comprises or consists of sequences from the gene DUT. In some embodiments, the sequences from gene DUT comprise or consist of:

(SEQ ID NO: 325)
TCCTAGCCAAGGGGTTCCCACTGCGTTTCTGTTGGGTTATCCCAA
ACCTGTGATAGGATAGTAGCGCCCTCCCTGCCTCCGGTCGAATAA
ACACTACAACGTCTGACACTCGGTGCTGAAAGGAAATCGGGCGCG
GTGTCTTGCCCACAGCTTGGGGAGCGGCCCCGGGCGCAATAACTG
TCACCGGCGCCGAGATGCGGTTCCGGCGCTTAGGGCGCCGCTAAA
CTCAGAGCCCGGGAGTCATGGCTGCGGGCGGTGCCGCCCCAGGTA
AATCAGTCCAGGAGCAGGGCCCGGGCCTGGCGTACACTCTCGGAA
AAATGGGGGCCAGAGCAAACAAGAAGAGCGAAAGCAAGAGGGCTA
GGCAGCCAGAGGCGGCAGCAAGACTCAAGACGCCAACGGCGCCGT
CTTCCTGGGGCCCCAGGGCCTGCGCCATCCCTGGGCTGCCGGGGC
ACCGCCTCTCCACGCCCCTCGTCCGGCGGCGGCTGCGACTGCTTC
CGAG

In some embodiments, the intronic domain that promotes trans-splicing primarily in the brain comprises or consists of sequences from the gene DUT. In some embodiments, the sequences from gene DUT comprise or consist of:

(SEQ ID NO: 326)
TGCCGAGGACCCAACCAGCCCAAACTCTGGGGGAAATGACTCCCC
TCTGCCCTCGCCCCGCGCTCTGCTACCATTTCCTTACGTCTCTGC
TTCGCTCAGCGATGCAAAACGCGCGAGGCGCACGGCAGAGGGCCG
AAGCCGCGGTACTCTCCGGGCCAGGCCCGCCCCTCGGCCGCGCCG
CGCAGCACGGGATTCCCCGGCCGCTGTCCAGCGCTGGCCGCCTGA
GCCAAGGCTGCCGCGGAGCCAGTACAGTCGGGGCCGCTGGCTGGA
AGGGCGAGCTTCCTAAGGCGGGGGGAAGCCCGGCGCCGGGGCCGG
GTAGGAAAGGCGGGGGAGGGGCTCCGGCCGTCTGGAAGGAATCCA
CGCGGCTTGAGGCTGTGGGGGAAGTAGGGTGGCGAGCGGTCCTTC
TGCGCGCGGGGGGCGGGGGGGGTGGGGTGGTCCATTAGGGTCCCC
TGGCGAGGGGGCGGCTTTCTAGTGTGTGAGGGCGACGCCCTAGAA
GCTC

In some embodiments, the intronic domain that promotes trans-splicing primarily in the brain comprises or consists of sequences from the gene SPG21. In some embodiments, the sequences from gene SPG21 comprise or consist of:

(SEQ ID NO: 327)
GTGAGTCTCCAGCGGGAGGTGGGGGCGGCACAGGGCTTAACCGGG
GCATCCTCCTGTAAACCCATCCTGGGGCTACACGACTGCAGCCCA
GGAACATTTCTTTCTTTCTTTCTTTCTTTTTTTTTTTCTCAGCAA
ACTTTTAAGAACTGTGAGCAGAGGCCAGTAATTTTGGAGACAGGA
CCTCGGCCTGAAAGTGATAGATGTCTTTGTTGTTACTGAGTGTGG
GCCAGGTGTAAGGTTTGTAAACGGGGGATGAGGAACAGAGCGGGA
CCGGAGTGTCTCATTTTTCCTCTTCTCGTTTTCCTGGGGAGTTAT
AAGTTAGACATTTATGCTGCTTCCTTTGTCTCCACCTACACTGGT
TCAGCTCCTTTTGGCACAAAGATTCAACAAATTAAGGAGTAACTT
TTTTTTCATCCCAGCTCACTGCAAGCTCTGTCTGCT

In some embodiments, the intronic domain that promotes trans-splicing primarily in the brain comprises or consists of sequences from the gene SPG21. In some embodiments, the sequences from gene SPG21 comprise or consist of:

(SEQ ID NO: 328)
TCCCCCCGCGCCCGTCGCCGTGGCGACCGCCGGGGCCACGCAGCA
CCGCCCCTTCCTCGCGACGTGGCCCCGCCCTCTGAGAACCCGGAA
GCGGGAGTCTCGCGCCAGCTGAGCGGCGGTCGGGCGGGGGGAGCG
CGGCGGCCCAAGCTCGCTCGGCCACGCGCCCCGCCCCCTCAGAGG
CCGGCCTCCCGCACGCACCGCGCAGCCTGCTGTGCCCGTGGGTCC
CGAGTGCTCCGCCGCCCGCCCCGACCCGGGCCCAGCCGCCTCCAC
GGCCCGCGCTCGTACTGGAGCGAAGAGCGGCCTCCTGAAGGAGGG
GAAGGGACGTGGGGGCGGCCACGGCAGGTGAGGGTGGGCGAAGCC
CTGGGAAGCGGCTGGGTGTGCGGTATGCGGGGTTGTAGGTGTGTG
TGTGTGGCGCGTGGCTGCCTTGGTTGGTGTGACTTTGTGAGTGTG
TTCTGAGTGGGCCGGTCCATGTGACCACGCACGTGATCACGTGCC
CTTG

In some embodiments, the intronic domain that promotes trans-splicing primarily in the brain comprises or consists of sequences from the gene PKM. In some embodiments, the sequences from gene PKM comprise or consist of:

(SEQ ID NO: 329)
GTAAGGGCATCCCTTGGCTTCTTAATCTACGTACTCCAGACCCAGG
GCTTGGACCTTTGCTGGTACGGGTTAAAGTGAGGCCGACAGTGAA
GGTCCACGTGGAGAGAGACATTGGGAAGTTGTCAAAAAGGCGTTT
AGAATCAGGTTTGACTCATTAATTTCCTGAGACTCTGAAGGAGTT
CAGCCTCTACACCTCAGTTTCCCCTGGGAAAACTGGGAGTAACAT
TTTCCTCATGATGGTTGAAAGGATTCATGCTTACGTGGATGTGGC
ACACTCAGTAAGTGTCTAATCTCTGGCGAAATACCCAGAAGAAAA
GCCTGTGACTGAATATATTATTTTTGAGCACAATTACTTAGTCCA
CAATTGAGCATAAGGGCCTTGATCCATTGTCTTCCTACTGTCCCC
AAACGCCTACAGCTAATCGTGAGGAATCAGGCTCTCTGAATAACC
ACGTGTCTCCTGGAAACCAGTTCCCCAAGGGATCTGGTTTAATAT
TGA

In some embodiments, the intronic domain that promotes trans-splicing primarily in the brain comprises or consists of sequences from the gene PKM. In some embodiments, the sequences from gene PKM comprise or consist of:

(SEQ ID NO: 330)
CGGTGCCGTCCCCTGTGGCCGGGATTAGATGGGCGGCCTGCGAGG
GCCTGGGAATGGCTCGGGGCCCGAGAGCTCGACCGGCCCTTGCCG
GGTGGCCGCCAGGGACCACGCTCCATCTCGCCGCGGCCGGGCTGC
ACGTAGCGGCCGCGCCCAGGGCCCACCCCGCTTCACCGGGCGATG
GCCTTGGGCCTCGTAACGGGCGGGATAAACCTCTGCAGGCTTTGC
TGGGGCCTCCTGGCCCTCGCCCGTTCCGCGCCTCTCCGGCACGTT
TCTTTCTTTTCCTTTCTTTTTTCTTTCTTTTTTTTTTTTTTAATT
ATCCGGCACATTTTTTAACAAATGCGTCCTGATTGTGGAACGCGG
AGGCCGCGGGGTGGGGTGGGGATCTGGTTACGGAGGGGGCAGGAA
ATCTGTCGCGTTCACTGAACGCAAACGGTGTGGGTCAAGGGCTGT
TTGGGGGTCAGAGTTAGAGACCAGGATGACTAGACGAGTCATAGC
CCAC

In some embodiments, the intronic domain that promotes trans-splicing primarily in the brain comprises or consists of sequences from the gene NPTN. In some embodiments, the sequences from gene NPTN comprise or consist of:

(SEQ ID NO: 331)
GTGAGTCCAGCACCCTACAGTAGTAGTACTGTAGTCATTAGAGGT
GGCCCAATGACCCTTCCCTTCTGCATCCTTTCCCTCTTCCCCAGT
ATGATCACTCACCCCACCCCTTGTCTTCGTTTTTTTTTCAATCCC
ATGACCCTTCTGGTTGTAGTGTATGAAAACATCTGTGGCAACTTT
TTTTCTTCTTTTCTTTGCCGTTCTCTCTGCCTCCCTTATCCCCCA
ACCCTTCTCTGTGTCTTTCTTCTCAGGAACAAAGAACTTGAGGAA
ATCCGTGACTGTCCAAAATCATCTGTGTTGGGAAGAGTTGGGGAG
GGAGAACTGGGAAGCTTAAATTAAAACTCATAAAACCAGGCAGTC
CAAGGAGGGTAGTGTTGAAATGGTTTTCCCTCCCCTTTGATTTTG
TAGTAGGTTTCCTGCTATACTTTTGCTGATTTCTTCTCTGGTGCC
TTTCTATTTTTGTTTCTCTCCCCAGTCTAACTACTTTTTATTTCT
TCTG

In some embodiments, the intronic domain that promotes trans-splicing primarily in the brain comprises or consists of sequences from the gene NPTN. In some embodiments, the sequences from gene NPTN comprise or consist of:

(SEQ ID NO: 332)
TTGTTGAGGCTTGCAAAAGACACTGCATTTCTAACAAAGCGTTTG
CCCATCCAAAAGAGAGATCCTGATTTGGCATATAAAACTACTGCC
TCTTCTGGTTATCTTTCATCACTTGCCTGAGTTTAAAAAGGTAAT
AAATGGTTTGTGTTCTAAGGAAAGAGGCTCTTCCCAAGACCTGAG
GCTTTCTCATTCCTACCAGCTGGGCCCATTGCTAAAATGCTTTCC
AGTGGGCTTGCTTCTGCCTGGCCAGCACCTGGGCTGCCCCAGCTT
GTAATTAGCCTGCCTTTGCTTCATAAGCTTCCCAGGGTGTGGTTC
TTCTTCTAAAAGCTTGTATGAATTGCTGTACTGTCTCTTTATAGT
TTAAAAATGAATCCTTTTTTGTTCTTTGCAACTCATTTGCTTTTT
TCTTTTCTTTTCTTTAAATTACTATTAACACCTGTTGATTCTTTT
TTTTTTTTCTGATTGGCCTGTCACTGCATTCCTGCTCCCCCTCCC
TCTA

In some embodiments, the intronic domain that promotes trans-splicing primarily in the brain comprises or consists of sequences from the gene COMMD4. In some embodiments, the sequences from gene COMMD4 comprise or consist of:

(SEQ ID NO: 333)
TACCTGGGTGCTGGTTATGTGAGGTGTACAGTTGATGAAAACTTA
TTAAGCTTTATAATAGGTACAATTTTCTGTATGTGTAGTAGACGT
CAATAGTTTTTAAAATGTCTTTTGCCACAGCCCGCAGCTGTGAAT
AATGCTATTCGCTTAACAGTGGAAAACGTGTCAATGACCTGAAAC
ACCCTGGCTCCCGGGATCCTACCCCCTTAAGTTAAACAAATGAAA
TAACTTCCACTGTCTTTCCTAATGTCTTAATTCGGTCCTCAGTTA
AACTGGCGGTTGGCGCCAGTTTTTTCCAGGGAAGGATTCTGGGAG
ACGGAGGAGGAGACGCCAGCATTTCGCCCCCTGCACCTCCAGCGG
GCTGCGGTCCGCTCCCGGCCTCACTCGAGCAGGCCCCGCCCTCCC
AGAGGCAGTTAGTCCAAGTCACGTGATCGTCGACTCAGCTGACCC
TGCGGGACCGGAAAAAGAAATTCCCGGGCCCTGGCTTCTTGGCGC
GATG

In some embodiments, the intronic domain that promotes trans-splicing primarily in the brain comprises or consists of sequences from the gene COMMD4. In some embodiments, the sequences from gene COMMD4 comprise or consist of:

(SEQ ID NO: 334)
TAGGGAAAAGTCCACCACTCTCCCGCTCCCGAGACGGGGGCGGGG
GTACGGGGCGGGTAAGACAGAGCAGGCCGGCCGGCTTAGAGTCCC
GGTGCTTCCCTGGCGGAAGGAAGGGCCCCTGCCTCCCGGGGCAGG
AACTAGGGCTTGTCTGGAGCTGGGAGTCCTTTCAGGTCTTCCCCA
TCCCCAAGAGGACCTCCCAAGGATACCCCCTTCCCCAGCCCTGCC
GTGGGGCTTGTACAAGAAGGTGCTTAGAATCAGGCTCACTCTTCC
ACACTGTTAGGAAGCCCCTCCGCTCTTTCCAGAGCCAGAAAGTAG
TAGTTTTGGGGTTGAGACTTATCCATCCATCCATCCAATCCATCC
ATCCGTACGTTCTAAGCGCCTGGTCTATACCATGAAGTGTGCTAG
GCACTGGGAGGACTTGAGCTGCCAAGGGAAGGGGAAATCGGAGGC
TTGAATTGGAGTCATAGCTAAGGCTCCAGGGGCAGAGACCTAACT
GCGC

In some embodiments, the intronic domain that promotes trans-splicing primarily in the brain comprises or consists of sequences from the gene MORF4L1. In some embodiments, the sequences from gene MORF4L1 comprise or consist of:

(SEQ ID NO: 335)
CTCCGCCTTAACCTCCCACGAAATCAAATCCCCGCTTTACCAACC
CGCAGCAGCCGGGGGCATGGAGAGAGAAGCGGGGGTGGGGTGGGG
TAGGGTAGCCGGCCCCTCCCCCACCCGGGGGGGGGGGGGGGGCGC
CTCCACCGACTCCGAACTACCAGTCCCGGCGTGCCCTGGGGCGGC
GCGGGCGCAGGGGCGCGTGCGCGGCGGGCTGTCGTTGGCTGGAGC
AGCGGCTGCGCGGGTCGCGGTGCTGTGAGGTCTGCGGGCGCTGGC
AAATCCGGCCCAGGATGTAGAGCTGGCAGTGCCTGACGGCGCGTC
TGACGCGGAGTTGGGTGGGGTAGAGAGTAGGGGGCGGTAGTCGGG
GGTGGTGGGAGAAGGAGGAGGCGGCGAATCACTTATAAATGGCGC
CGAAGCAGGACCCGAAGCCTAAATTCCAGGAGGGTGAGTGTGCGC
CTTTGGGAAAAAGGCACCTAACGGCGCAGGAGATAGAGGCGGGCT
CGAG

In some embodiments, the intronic domain that promotes trans-splicing primarily in the brain comprises or consists of sequences from the gene MORF4L1. In some embodiments, the sequences from gene MORF4L1 comprise or consist of:

(SEQ ID NO: 336)
TGCGTCATTGTGAGAAAGCGCTTTGTGAAGCGAGAGTGCTGCGCA
GGCGTTAGAGTGGGAGAGAGAGCGTTTGGCGCGTGGCACACCCTC
GTCAAGTGTTTGTTATTGTTCTGAGGAAGAGGGCGCGGAGAGAGC
AGCCTATTGTAGGGAGGCGACGGGAGCGCCATGGCGGCGGTTAGG
GGAAGTGATGTGGCGGCGGGGGGGGAGGGGCGGTGGCTGTTTCGG
GCGGTCCTGCGGCGCGCCCGGCCGCTTGGGCAGCTACGACGGGTT
TTTCGGTGCTTCCTGGAGCCCCGGGGTGGTTGCGTTCGGTGGCAA
CTCAGCTGCGGATGGGGGTGGGTTTGGCGCCACGGGGCGGGAGAA
GTGCGTTGTAAAATGGCCGTTGCAAGATGGCGGCGCCATCATGGC
CGCCGGCGTCTCGGTCCCGTATGGAGGCACAGCTTACGGGCAGAT
GCAGCGCCCCCTTCCACGACGACCAGAAGGTTGCCGGGGCCCTCC
GCAT

In some embodiments, the intronic domain that promotes trans-splicing primarily in the brain comprises or consists of sequences from the gene AP3B2. In some embodiments, the sequences from gene AP3B2 comprise or consist of:

(SEQ ID NO: 337)
GCTGCTGGCTCACCTTTCCTTCTAGGGGTTTGTCAGGAACAACTC
TTGTTGAATCCCCAAGGTAACCATCTCTGGGTCAGGATGCCTAGG
AGTCTGCAGGCATCTCTGTGGAAGAGACATCTGGGCTGGGGTGGC
AAGGCAGAGCTGGAAGAGCACCTTGGTCCTTTTTATAGCCTCTGC
AAAAACAAGTCCTCTTCAATGGCTGGCAGACCCCCTTAGTTCTTC
TTACGGTCCCTTGGGAAGGAAGGACTCATAACATGGGGACTTCCC
CAAACACAGGTGCTCAGTTCAACATATGTCAACTACTCCAGTTAG
GGGGCCTGGAGGCCCCACCTGGCTGGGGTACTGACCCACTGGTCA
TCTTAGTGGAAGAATAGAAGATGCATAACTGTACTTCTCAAACCA
CTTTGTGGCAAGTCACCTGGGGATCCCATCACATGCAGATTCTAT
TAGTAGGTCTAGGGTGGGGTCTGAAATTCTGCATTTTTTTTTTAA
GTGA

In some embodiments, the intronic domain that promotes trans-splicing primarily in the brain comprises or consists of sequences from the gene AP3B2. In some embodiments, the sequences from gene AP3B2 comprise or consist of:

(SEQ ID NO: 338)
GATAAAGGCCTGGGTGGGAGGGGTAGAGAGGCTGACAAAGGCTTC
CTGAGGGAGGGCCCAGTCTGAGAGGGAAGTACCTTCTCCCCAAGA
CTGATGATTTAAATTCTGACCTGCCCTTTGCATGATACCTTCCCA
GCCTGGGAGAACTTGTTGCTCAGTAAGCAGACCCAGGTGCTGGCC
TCTGTACCCACCATGGCCTCTGCCCTCATCACGACCTCTACCCCC
AGGAAAGCTGATGGGCATGAATGAGATCACAGAGAAACTCATGCT
GCCAGACACCTGTCGGAGTGACCACATTGTGGTGCAGAAAGTGAC
TGCCACTGCCAACCTGGGTCGTGTTCCTTGTGGGACATCTGATGA
GTACAGGTACTAGGCCACCAGTAGAGGGGAAATGATGAGCATGGG
GGCCCTGGTTTGAAGGAGGAGCCCCAGCTGCTGATCCCCTTCCAA
ACACAGTCACCCTTAGCAGCTCTTCCTCACCCCTTCTATTCCCAC
CTCA

In some embodiments, the intronic domain that promotes trans-splicing primarily in the brain comprises or consists of sequences from the gene SEC11A. In some embodiments, the sequences from gene SEC11A comprise or consist of:

(SEQ ID NO: 339)
GTGATTATTTAAACTCTCCAATCTCTTTGAGTCTTTGTTTTTCCT
CAAATTATAAATGTACAGATTCATGTAGAAACATAAGAAAATATC
AATAATCAAAAAGCAAAGTCACTTCTTGTCTCCTTTTTCTTGATC
TCCATAAATATTTACTATTAACTTTTTGGTTTATATTAGCCTTTG
TGTGTAGAGATAAAAATATTAATGAGTACTTTTTTTTGCCTTTTT
TTTTTTTGGAGACAGTCTTGCTCTGTCACCCAGGCTAGAGTGTGG
GTGGCATGATCTCAGCTCACTGCAACCTCTGCCTCCTGGGCTTAA
AAGATTCTTGTGCCTCAGCCTCCCGAGTAGCTGGGATTACAGGTG
TGTGTCACCACACCCAGCTAATTTTTTGTATTTTTAGTAGAGATG
GGGTCTCGATATGTTGCCCAGGCTGGTCTCAAACTCCCAGCTTCA
AATGATCCACCTGTCTTGGCCTCTCAAAGTGCTGAGATTACAGGC
ATGA

In some embodiments, the intronic domain that promotes trans-splicing primarily in the brain comprises or consists of sequences from the gene SEC11A. In some embodiments, the sequences from gene SEC11A comprise or consist of:

(SEQ ID NO: 340)
CAGAACAACCCTAATATTCCCATTCTACAGATGAGGAAACATAAA
TGAGTTAGGTTATGTATCTAAGATTATACTGATGGTCGTAAGTGG
CTGGCTGCATAGTCCACCTTACCATCATACAAAAATTACTAGCGT
AACATTTCTGAAATGGATAGTTGCTAATTCTCACTAGTTAGATGC
CACATGGCACTTTTGCCACTGTCAGAACCTCTGCTCCTTTTGACC
TAGAACAGACTGTATGGATACAGAGTTATTACACCAGTAAGCAGG
TCTGAGATACGCAGTTCCCATAGAGAAGGAAGTATGAGTTGCTAA
GTGAAAGCTTCCTCAGTTACAGCACGAGAGATGCTCAGTTTGGGG
CCAAAGAGATCAGATCAGAACAGGTATCATTGTGCTTTGCCTGCT
GCAGAGCTGTGGTTGTTATTGTTGTTTTAATATGTCCCCGCTTTA
AGGAACTTTTCCTTTGATGGAGCTAAAAATACTTTTATAATCCAA
ATTA

In some embodiments, the intronic domain that promotes trans-splicing primarily in the brain comprises or consists of sequences from the gene PRC1. In some embodiments, the sequences from gene PRC1 comprise or consist of:

(SEQ ID NO: 341)
GTTAGTACAAGAGTGAGGTCTTTGGTTTGTTTGTTTGGAGACAGG
TCTTACTCTGTTGCCCAGCCTAGTCTCAAGTTCCTAGGCTCAAAC
AATCCTCCCAACTTGGCCTCCCAAAGTGTTGGGCCACCACACCCA
GCCAAAAGTGGGGTCTTCATGTGGCCTGCTCACTAGTTTTTAAGA
CATCCAGACATTCAACAGGAAGTATATCCTTTTCTGAACAGATAA
AGCCACCTTTTCAAGACATTTAGCATAATTGCCATTCCTGTATGA
AAAAGCATTTGCAAATTTTATTGGGAAATATTGGTCTCTTGCTTT
CTGAAGCAATATGTCACTGTAGCCTCTAGCTGTGAAGCTTAAGTT
TTTATCAGGTGCTTACTTTTTGCCAGGTGCAGTGTCAAACCCTTA
ATATTATTTCACATCCTCATGTCAAGAGGTGAGGATACTAAAGCA
CATAAATGCTGAGTCATTTAACCCAGAGACATATAATGGTAAATG
CCAG

In some embodiments, the intronic domain that promotes trans-splicing primarily in the brain comprises or consists of sequences from the gene PRC1. In some embodiments, the sequences from gene PRC1 comprise or consist of:

(SEQ ID NO: 342)
AAAGCGGTAAGAAACCCAGTGGGTACTGGGTCAGCAGTCTTCTCT
TAGCACTTCTGTGTTAGTCATAAAGACTGTTGTTGATTTGGCCAG
GCTCAGTGGTTCATGCCTCTAATCCCAGCACTTTGGGAGGCCGAG
GCAGGTGAATCATTTGAGGTCAGGAGTTCGAGACCAGCCTGGACA
ACATGGCGAAACCCTGTATATACTAGCCAGGTGGTAGTGGCGTGC
GCCTGTAATCCCAGCTACTTGGGAGGCTGAGACAGGAGAATCGCT
TGAGCCTGGGAGGTGGAGGGTTGCAGTGAGCCAGAATCGCGCCAC
TGCACTCCTGTCTGGGCGAGAGAGTGAGACCCTGTCTCAAAAAAA
AAAAAAAAAAAAAAAAAGTGTTGATTTATAATACCTTTGTGGCTT
TGCATTATAATTTTACTTAAAAGGGGGGGATCTTTTGCAAACCTG
AGTGAATATAAAACTATGGCAGCCACCCTTAACAAGTTTCTTTCT
TTCA

In some embodiments, the intronic domain that promotes trans-splicing primarily in the brain comprises or consists of sequences from the gene MT3. In some embodiments, the sequences from gene MT3 comprise or consist of:

(SEQ ID NO: 343)
TAGGCCTGGCTCGGCCTCAGGCTTATTCTGGGACCCAGGCACATT
TCTGCTTCTCTGGCCTCACTTTTTCTTATCTGTATAATGGAGGGT
TAGGAGTCAAGGATCTGAAGATGCCAGGCAGGACAGCGGGGAAAT
GGAACACGCCCCTTGCCAGTGACACTCGGAGGGCGCAAGAGTCAG
TGTCGGGCTCATCGTGAGCAGGTTCTTTCTGCCCCCAGTTCAGCA
ACCCGTCTGAACAGATCTGGCGTCCTGGAGAATTCTAGAATGAAG
GGGAAGAGAGGCAGGGAAGAGCTGGGAAATACGCAAAGCGCCTTT
TTCTCCACTTTCGGAGATGGTACGTGCGCGCTTCCACGCAGTGGC
GGCTGCTGCGGCGAGCACGTCCCCTGCGGGACCCACGCGGGGAGT
GGGCTGGCAGTGCGCGCACAGCGGCGGCGAGTGGGTCGTGCACGC
GGATGCGGGGTGGGAGTGGGGGCGCACGCGCGGGCGTGGGCGAGC
GGGC

In some embodiments, the intronic domain that promotes trans-splicing primarily in the brain comprises or consists of sequences from the gene MAPK8IP3. In some embodiments, the sequences from gene MAPK8IP3 comprise or consist of:

(SEQ ID NO: 344)
CTGTCAGGCACACCCCAGAAGTCAGGGGTCCTTGAAGCCCCTACG
AAGCCCAGCCCGGGGTCCCCTCCCCACGTGGGTGGACAGGCCGAG
TCCTGCTCTCTGGCTGCACAGCAGGAGGCTCCGAGGCCCCACCCA
GAGGCTGTCCAGATGCAGCCCCCCAGCCTCCAGGCACAGCCCATC
CCCACAGGGGTGCCTTCTGCCCTCAGCCCCACCCAGCCCAGAATT
TGGGGTGAGATGTTAGCTTCCTGCTCCACAGGTGACACTGGCCTG
GCAGTCTTCATGTAACCCCAAGGATAAACTTTCCAGAAGGAATAG
CTGGCTCCATCCCATCTCCTGCCCTCCTAACTCCCTCCTGCACCC
ACCCTAACCCAGCAGACCCAGCTCTGAATTCCTCCCAGCCTGCTG
TCCACACGGGCTTAACGCGCTTCTCTTCTCTCCCTTTCTCCCTCT
CCTCCCGCCCTGGTTGCCCATCTCCTGTGGGACGGGGACGGCTCC
CTAG

In some embodiments, the intronic domain that promotes trans-splicing primarily in the brain comprises or consists of sequences from the gene MAPK8IP3. In some embodiments, the sequences from gene MAPK8IP3 comprise or consist of:

(SEQ ID NO: 345)
TAAGGCTGAGGCCCCGTTCCAGCGTGCGTCGCTCCTCCACCCCGA
CATGGTCTCCTGCTTCATGAGCCGTTTGCTTTGTTCTGCATGATG
GGGACAGTGTTGGGGCCGCCGGGGTCAGGGGGGCAGCCCTGAGTG
AAGCTCGCTCTGTCCAGGCTGCCTCTGGGCTGTGGGAGGTGGCGA
CTTCCCCAAGGGACAGACTTGAAGGCCCGAGGAGGGCAGCCCCAG
CCTCTCTGAACCCCACAAGCGCCGGGCAGACCCTGCCTGTGGGGA
GGAAGGCCCATGTTTGAGGCAAGGGAGGTCATAATAGAGACAGCA
GGCAGGACCCCTGAGCCCAGGCACAGAGGCGTGTGATGCTGTTTC
ATGACTGCATACACAGTGGGGGAGTGTGTGGGAGGGCAGGACCTT
GGAGTTTCTTTCCATCCTTTTGGGGTTTCTGGGGCATGCTTGCAG
GGAGATCAGTCCAGGTCCACTTGGGGCCTCGTGGTAGAGGAGCCT
TCAG

In some embodiments, the intronic domain that promotes trans-splicing primarily in the brain comprises or consists of sequences from the gene HMOX2. In some embodiments, the sequences from gene HMOX2 comprise or consist of:

(SEQ ID NO: 346)
CGGGGTTCCCGCCAGGAGCGACAAAGGACACCCCCATTGTCTATG
CATCGAGTATGTGTCGAAGGGGGCGGCTGGGCCCGGAGCGGCAAG
ACGCCTGTCCCGAGATATCGGGGTCCCGCGACCCACCTCGTGTCC
CTTGGGCCCGATCCCCTCCTCCTTCCCGCCTCCCCCGGTTCCAGT
CACCCGCCCCCCGGCCCGGGTCCCACCGGCTGGAGTGACCTCCTC
GCCGCGGCCTGGGCGGGACAAGTTCCAGGAGCCCGGGACCCCGAT
TCTGCGGCCGAGTCCACTGCAGAGGCGGGGATGGAGCTGCGCGCT
AGGCGCCAACTCCCTCCCCGCAGCTCCGGCCCCACCTGGCAGAGC
GCCGCTCACGGAAGCCCAGCGTCGGAGGACCCCTGGCTGCAGCCG
CCAATCCCGACGTGTCGGCCGAAACCCCTCCTCCGGCGCAGTTGG
CTCCTCCTGGATCCCGCGGGACTCGGCGTCGTTTCCGGGTTTGCC
CGCC

In some embodiments, the intronic domain that promotes trans-splicing primarily in the brain comprises or consists of sequences from the gene HMOX2. In some embodiments, the sequences from gene HMOX2 comprise or consist of:

(SEQ ID NO: 347)
TATTGGGCGTCAGGCAAAGATAATTAAAGCTATGTCCATGAGTCT
TATCCCTTGAGGCCTTGTCCGCTTGATATTTTCTTTGCCTAACTC
ACTTTTAAAATTTTATTATTTTTATTTTTTTGAGACAGTGTCTTG
GTCCGTCGCCCAGGCTGGTGTGCAGTGGCGCGATCTCGGCTTACT
GCAACCTCCGCCCCCCGGGTTCAAGAGATTCTCCTGCCTCAGCCT
CCCGAGTAGCTGGGATTACAAGCATGTGCCACCAAGCCCGGCTAA
TTTTTGTATTTTTTAGTAGAGACGGGGTTTCACCAGGTTGACCAG
GCAGGTCTCGAACTCCTGACTTAAAGTGATCCACCCACCTCAGCC
TCCCAAAGTGCTGGGAGTACAGGTGGGAGCCACCGTGCCCAGCCC
TAACTCACTTCTGAAGTTGCTGCTACTTGCACTGATTTCAGTTTT
TTTTTGTTTTTGTTTTTTGTTTTCAAGGTGGAGTCTTGCTCTGTG
GCCC

In some embodiments, the intronic domain that promotes trans-splicing primarily in the brain comprises or consists of sequences from the gene CDIP1. In some embodiments, the sequences from gene CDIP1 comprise or consist of:

(SEQ ID NO: 348)
GTAAAGCGGGGCCGGCGGTTGGGGCGCGGCATGGCGGGGCCCGTC
CTCCCCTCAGGGCGCTGCCGAGACCTAGCGCGGCCCTGGGGATGT
CGAAGCCGCTGCGGTGGCGGGGCCCGGGCGGGCGCCCCGCTGTGT
GCCGCGGGGTTGTGCGCCCGGAATGGCAGCCTGCTCGCCCGCACG
TTTTTCCGTCAGGGAAGGTTGTGGAAATTGCCATCTCCGTCTTGC
AGGGCGAGACGGGGAGGGGAGGGGTGGGAGTTGTTTTCTCAAGGA
AAGGGGGTCGAGGAGGAGCCCCGTGGCCGAGCCAGCCCCTGCCGA
GCCCCGCGGGTGACCTTGGCCGTCCCGGAGCCCAGCAGTGTCCTG
CACCCGCGCCCGGCTCGCCGACGTGTGGAGTCGGGATGTGGCGCT
CGGCCGCGCATCCCCGGCGGAGATGGCGACGGCCTGGCCTAGCTT
GGGGCTAGGAGTCGCGCCTTCGAGGACCTTGGCTTAGCGAGTTCC
TTGG

In some embodiments, the intronic domain that promotes trans-splicing primarily in the brain comprises or consists of sequences from the gene CDIP1. In some embodiments, the sequences from gene CDIP1 comprise or consist of:

(SEQ ID NO: 349)
TTCCCTATTCTCCAGCCTCGTGTACATTGGGCAAACAGGCCCCGA
CCATCCCACGTGCTCAGCCCCAGCTCTAGGCCAAATCGCCTCCGC
TCCCACTGACGTCATCACGGCGCATCACGTGCCTCCCGAGACCCC
ACCCCCCACCGTGACGTCCCGGGCCTCGGCCCTGGTCCAAAGTCT
ACCCGCCTCCTTGTGACAGAAGTGCGACTGCCAGCTGCCGAGGCG
TTCGGTCCTGCTGTTGCGGCCGCTGCCCCAGGGCTGCGGGGACGG
TGAGTCGACTGGACGCATTCGGAGACGGCTTCGCGCGGGAGCCCT
CCTCCCCGGGGGAAGGGTCTGGGCGCCAGCTCTCCTCAGGTCGGG
GCGTGACCTCGGACCGTGGCGCCCCCTCCCCGCCCCCGCCCGCGG
CGCCTGGGCGGTGCATGCCGGGCACCGTAGTCCCCAGCCTCTCAG
AGCGGGTTGTGGTGGGGGGGGTGACCGGGCGGACTACAAGTCCTA
GAAG

In some embodiments, the intronic domain that promotes trans-splicing primarily in the brain comprises or consists of sequences from the gene SEZ6L2. In some embodiments, the sequences from gene SEZ6L2 comprise or consist of:

(SEQ ID NO: 350)
GTAAGCACCTTGCCTGCCCCTGAAGAGCCACCACTTGGCTCTCCC
AGGACTCCATAACCTTCTCGCAAGGTCTCTGGAATCTCTGTCTTG
GGAATTTCAAACTCAGCTGCCCCCAGAGGCCATGTCAATGAGCAT
AGCAAGCCATGTGTATACAATAGGGAGTGGTGAGGACTGTGGCTA
AGTGGAGGACTTGTGCCCCATCTCAAGAATACGGCTCTACCCCAT
AGTCGAAACTGAGGCACAATTGGCAGATCTTCCAATTATTCAAGA
CAAGCCAGAAATTCAGATTTTTGTGTCATTTTACGACACCGTAGG
ATGAACCAAACACATTCACTGGCCCCCCAGCCCCTAGAGGTCAAT
GTTACTTTAAAACCCAGGATCGTTGCCCCTCCTCCCATTCGGCTG
CCTCTTCCACCCCTATCAGACTCCAACAACTGACCTGATCTCTCT
GTCTTCAGGATACGCGGGAGTATGAAGTTTCCATCTGAACCCCAA
GACT

In some embodiments, the intronic domain that promotes trans-splicing primarily in the brain comprises or consists of sequences from the gene SEZ6L2. In some embodiments, the sequences from gene SEZ6L2 comprise or consist of:

(SEQ ID NO: 351)
CTGGCTCAGACCCCATCCCCTCCCCCTCCTCTCCCTGCAGTTGCC
TATGAGGAGCTCCTGGACAACCGAAAACTGGAAGGTCAGTGAGAG
CACAGGTGGAGACCCAGGCTGGCCAGGTTGGGAGGGCAAGGCAGA
GGCTGGGGCAGGAGGCCAGGCCTTGGAGGGATGAAGCCCAGGGTT
TGAGGCCCAGGGCAGGAGGCTGGGAGAGCCCAGGACCGGGCCCAG
CCACAGCTCACCGGCCTCTCCTCCTCCTGTAGTGACCCAGACCAC
AGATCCATCACGGCAGCTGGAAGGGGGGAACCTGGCCCTGGCCAT
CCTGCTGCCTCTAGGCTTGGTCATTGTCCTCGGCAGTGGCGTTTA
CATCTACTACACCAAGTAAGTAGCCTGCTTGGGGACTGCCGGCCA
GGGCAGACGTCTTGCGAGCCTGGCACTGGAGCCTGGGTCAGGGGA
GCTCAGGGGCTTACCTGTTGATAAGCTCCTCTGTCCTCTCCCTTT
CCCA

In some embodiments, the intronic domain that promotes trans-splicing primarily in the brain comprises or consists of sequences from the gene ALDOA. In some embodiments, the sequences from gene ALDOA comprise or consist of:

(SEQ ID NO: 352)
GGAGGCTGAGGCAGGTGAATCACTTGAACACGGGAGGCGGAGGTT
GCAGTGAGCCGAGATCGCGCCACTGCACTGCAGCCTGGGCGACAG
AGCGAGACTCCGTCTCAAAAATAAATAAATAAATAAATAAATAAA
TAAATAAATAAATATAAATAAAATATAAAAAGTAGCATTGATGTT
TACAATATCCCTGAGCGGCAGGCGTTTCTCCCTTTTGCAGATGCT
CAGTGGCTTGGCCGGAGATACATCCAAGTGGTGGGTGTAGCCTGG
GGGTTCAATCCTCCTGTGGCGCCCCAGAACCCGGTGCCTCCTCCA
ACGTCCGGCATCTGATGAGGATCCGACCCAGGCGGGCGGCGGCGG
GATTCGCTCTTCCCCTTCGCTCCCCGCGAGAAAGCCCCGAGGGCC
GCGGCGGCGCAGAGCCGGTGACAGTTGAAGCTTAGGCGGGAAGAG
GGAGGCGCGAGGCGGGAAGAGGGAGTTTGGGCCTCGGCAGCCGCC
GTAC

In some embodiments, the intronic domain that promotes trans-splicing primarily in the brain comprises or consists of sequences from the gene ALDOA. In some embodiments, the sequences from gene ALDOA comprise or consist of:

(SEQ ID NO: 353)
TGATGGGTGCTGACCGACTGGGGAAGCCCGGAGTGTGGGGACTGA
GGAGGGGAGTGGCCTGGGGCGCGCTGGAGCCTGCGAGGAAGGCGC
CGCCTCGGATCCCCCGCCCCCCATTCCCCTTCCCGAATTCCACCC
TCCGGCCCAGCCATGGCCTCAGTTTCCCCAAACAGGAAAGGGAAG
GAGGGTGGGCACCCCGGTCTAACGGTGCCTCTCAGCCTCTGAGAC
CCAGAACCTTCCTTCTGCAGCTCCCGGACTGACTGGCTCTGCCCT
TCCCCATGGACGTAAGTCCGGGCCCCTTCCCCTCTCTGGGCCCCC
TCAGCGCCCTCTCCTAACCCCGAATGGGCCTGTCTTCCAGCACGG
ACTGGGGAGGGGGCTTCTACCCAACAGAGACTACAACCTGCCAGC
TCAGGACGAGAGCTGTCAGGAAGAGTCCAGGAATGGACTTCCCAC
GGGAGGGCACATTTCTGGTATTCCTGGCAAGATAAGGAGTTGACT
AAGT

In some embodiments, the intronic domain that promotes trans-splicing primarily in the brain comprises or consists of sequences from the gene GPT2. In some embodiments, the sequences from gene GPT2 comprise or consist of:

(SEQ ID NO: 354)
CGCTGGGCCCCGGGGAGGCTGGGGCCGCTGAGCAAGGGAAAAACC
GCAGCAGCCCCAGCTGGGGTCCTTCCCACGACGTCCACCCCCATG
GCCAGCTCCTCAGTCAGCCAAGCCTGGCTAAAACGAGAGAATGCC
CCCTCCCGCCCGTTCACTTACTGGTGCCCAGCACCGGGCACTCAG
TGAGGCCTTGCAATACGGTTCACTTGTTGAATTGAATTCGTCAAT
TGTTGAGCGGGCCGTGCTGCCAGGCACCGCACGTTGTGTGTCTGC
CCTACTCTCGTGGAACCAAGGTCCAGTGGAGAGAGGAACATAAGC
GAATGAAGAAAGAAGCAATAGAGTCTCAGTGATAAGTTCTTGGAA
AAAAGTAAAACGGAAGAGGGTTGGGATGGGGGCGGTGGGAGGGAA
ACCATTTTAGGTAGGAGGGGAGACGGGGTGGGGGGGGCTCTGCGG
AAAGTTGGTTCTGTTCCCCAGTCTCTTTCTCCGTGTCTTTGGCCT
TTAG

In some embodiments, the intronic domain that promotes trans-splicing primarily in the brain comprises or consists of sequences from the gene GPT2. In some embodiments, the sequences from gene GPT2 comprise or consist of:

(SEQ ID NO: 355)
TGGGGAACCAGAGAGTTGGGCCTGTCGGGCTTTGGTAAGGGGGCC
CTGGGACTTTCTTCATCTTGGTGGTCGGCCGGCTGGCTAGGATCT
TGGCTTGAGTTTATAGCTTCATGTAAGGTGAGCCCACAGCCAGGA
AGGATTTCTGCCTCCCCACAACCAGGATGTCTGGGGTGGGTGATA
GAGGGGTCCCTAGGACCTAGGAGAGAGTTATAGCCTGGGTGGAAT
TCAGGAGGCAGAGCCAGGAGGGGTTATCTTAAAAAGTAAATACTC
CTAAGATTTGTAAGAAGCTAATAATCACCTAGCTGCTTGCTAGAG
AGAGTCTGACTGAGAATAGAAGTGGGAAGGAGGGTTCCTTGATCT
TGCCCTCACCCGGGTCACTAGTGTCCCATGAATGCATGGCAGTAA
GCTTCAGGGGGATCATGACACTTGGTCCCATTCCAGTGGGGACTT
GGGGGTTTTAATGAGCTCCTGGGCCTCCTGAGACCTTGTTGAGAC
ACTC

In some embodiments, the intronic domain that promotes trans-splicing primarily in the brain comprises or consists of sequences from the gene GNAO1. In some embodiments, the sequences from gene GNAO1 comprise or consist of:

(SEQ ID NO: 356)
ACATAGCCCAGTTCTAGGGACTGCATTCCACCCTGTGTAAGAAGT
ATAGAAAATTACAGCTCAGCCAAGAACATTGATTTTTTTCTCCCC
CGAACAAAATAACCGAGTTTTCAGGATGAAAGCAGAGCTGCTGGT
TGAGTTGGAGGGTGGAGAGCAGAGCCAGGCCCACTCTGCCTTCCT
GCTGGTTGAGTTGGAGAGTGGAGAACAGAGCCGGGCCCACTCTCC
CTTCCTGCCCCAGAAAACTCTGGGGCCCCATGGGCTGCAGCTTGG
AGCTCCTGCTTTAGGACATCCATGTGGTAGAAGGCCCCACGGGCC
CCAGGAACCCCAACTGTGCATGCTCAGGTCGTGGCCCTCCCTGGA
GCAGGCTGTGGGCTGAGCTGCGGTCCTGCTGCACTGGCTGGGCTC
TCATCACAGTCCCCGCTAGGGGAGAGCCCTTGGCTGGCAGAGGTC
TTCTGTCCCCACCAAAGCGTCAAAGCCCACCATCCACCTCTCCTC
ACAG

In some embodiments, the intronic domain that promotes trans-splicing primarily in the brain comprises or consists of sequences from the gene GNAO1. In some embodiments, the sequences from gene GNAO1 comprise or consist of:

(SEQ ID NO: 357)
TGAGTGTCCCAGCGGGCGCATGGCCTGGAGCCGGGCAGTGATGCG
GGAGTGGAAGGGACTGGTGATGGGGATTGGCAGAGCAAGGAGGAT
TCTCGGCTGAGGTCACGCCTGCCGGGAGATGTTCTCCCATAGGTA
GAGGGTGTCAGGAAGGAATGAGAAGGGGCCTCTCTTCCTGCACCC
CAGGAGGAGAAAGAGGCCAGAGGAGGCAGAGGACGCAAGAGACAA
CCAAACAGTGGAAATTCCCAGAGGGCCGGGACCTTGTCACTGGCT
TGTCCTGGAGAGTGCCTGGCACTGCAAAAAGACAGCTTTCAAGTG
TAGCAGCTTTGTTTTTTTATAGGGAGCCCCTGACCGCATAAATAC
AAACCAAAACAACAGCCATATAGGCAGAAAAGAAATGGTGCCATT
TTGTTTTATTTTAGAAAAAATAATAAGCTTTCCATTCCCCATCTT
TAAAATTAAGAATAAGGAATATGTAAGTCTAATTAATATAATAAA
TGTA

In some embodiments, the intronic domain that promotes trans-splicing primarily in the brain comprises or consists of sequences from the gene MT3. In some embodiments, the sequences from gene MT3 comprise or consist of:

(SEQ ID NO: 358)
TGAGCCCCCGCCCCCGCTCGCATCCTGCGCACTGCGCGCCCTTGT
ACCTGCAAAGAAACCCACGCCCTGCGCCTTCGCTCAAGGACACTT
GGGGGAAGGGCCCCTGATTCCCTATTCTTCACCTCGTGAAGGGCG
GGCATGCCTGTGTCGCGGAGAACAGGGAGACTTGGCACCCCATCT
CCTCGTGACAGGCGTGGGGACCCGAGTTCGTCCACATTAACCCTT
CCTGTGGCGTCGCCCTCTCTAGGTGGCTCCTGCACCTGCGCGGAC
TCCTGCAAGTGCGAGGGATGCAAATGCACCTCCTGCAAGAAGAGT
GAGTGCGGGGACCCTTCCCCTCTGCCGCCGCCCCCTCTGCTCTGC
GGAGTCGGTGTCTCACCACGCAGGATGTGGAGAGACAGCCAGGCC
CCGATCCCGTGGTTTCTGACTCTTGCTGGAATAGAACCACCCGGG
CAGACATTAAAATACAGATGCCCCTGCCCCACACCCAGGAATTGA
TGGC

In some embodiments, the intronic domain that promotes trans-splicing primarily in the brain comprises or consists of sequences from the gene MT1G. In some embodiments, the sequences from gene MT1G comprise or consist of:

(SEQ ID NO: 359)
GTAAGGGACGCCCGGGTTCTGTGCCTTGGGATGCCAAATTCCCAG
ACACCATAGAGAGTGTCCCTGGGTTTGAGGAGGTCGTATTTTGCT
ATCAGAGGTAAAGGGGACTCCTTTATTGGTCCAGTGCTTTCCTGT
TGGCCAAGCTCCTGAGGGCATTTTCCTCCTCCCTGTTCCTCTATG
TCAGAGTTGAGGGTCCTGAGGCTCAAGGCTGTCCTGCTCCACGTC
ACCTGGTTGGTCACAGGGCTGCTGGCTGAGCCCCAATTCTCTAAC
CTGACTCTGAGCTACCGGATTGGATAGGAGACATTGGATAGGAGG
GGACATTGCCTCTTCCAGGTTCAGGACAGAAAGTCGAAGTCTTCC
TGGGCCATGTCTGCAGCCTGGGACTTTCCTTTGGAGTAGAAATAG
GAGGGTGCTTGGTTTTCCCAGCATGAATGGAGAGGACATGGGGCT
TCTCTTCCTCTGCTCTGAGTGGGAAAGGAGCTCTGAGGGCTGGCT
CTGC

In some embodiments, the intronic domain that promotes trans-splicing primarily in the brain comprises or consists of sequences from the gene MT1G. In some embodiments, the sequences from gene MT1G comprise or consist of:

(SEQ ID NO: 360)
GGATACTGCCAGAGATGCGAAGTTCGCACCCTTGGCCTGCCCTTT
GCACACTACTCCCTTGCTATGCTCCCCGCTTTGGCAGATCCAGAA
AGTGGAGCACAAGACTGGGGTTGCCCTCGACTCCAGGAAAGGCTT
AGCTATTGACTAGAGACCTGGGCGTTGGTGAAGGTGAGGGGAAAG
CCAGAATACTGGGGTAGGCGGAACGCCAAGGGGCGGGTGTAGCAG
GCAACCTCAGGGAAGCTGGGAAGGCCTGGCAGGGTGAGAGAAGCC
GCACACGGGGGGCACAGCGCCTCCCTCGCGAGCCGGTGCGAAAGG
GGCCGTCCTGCGGTGTGCGCCCAGCTGCGCTCAAGGGACCTTGCA
CTTGGCCCATCTCCTGCGCACAGCCCAGGCCGGGACCGCGGGCGG
TGCGGACTCAGCGGGCTGGGTGCAAGGGCGGGGCGGGGCGTCTGC
GCCCGGCCCCGTCTCCTGACTATAAAAACAGCCGCTGGCTGTTGG
GCTC

In some embodiments, the intronic domain that promotes trans-splicing primarily in the brain comprises or consists of sequences from the gene NUP93. In some embodiments, the sequences from gene NUP93 comprise or consist of:

(SEQ ID NO: 361)
TAATCTGAATAGTTATTAAACAGTATCAAAATCTTTTAACTACTA
AGAGGCCAAACATTTAAGATGGCGAATTTACTGTTCTGCTTGAAG
GACAGTACAATTGACAAGGGCACAATGATCCGAGATGCTTCTGTA
TGTTGTATCTACACATACTGTAGACTAAAATGGCAGAATCTGTCT
GCTGCTGTATAAAATCTTTTAACTACCATTTATTTTAGCATCATG
GCATGTGCAAATTCCTGCTGCTCAGTTAAGGGACCAGCTTCACAA
CTGGTGTTACAATCTGTGGCCACAGCACTGCATCACAATGCAGCA
AATGCATTTTCCACATTAACCAAACATGACAGCTGAAGCGATTAT
TAACCATGCAAGATGTCGGAAGTCATCAAAGATTCACATCAGAGT
AATTGCAGTGGTTTGGGTTGGGGAAAAAAAGTATCATAGCATTCC
TTGCCTACATGTTGTCTCTCCCCCTCTTAAATCTTAACTTGGTAC
AGTC

In some embodiments, the intronic domain that promotes trans-splicing primarily in the brain comprises or consists of sequences from the gene NUP93. In some embodiments, the sequences from gene NUP93 comprise or consist of:

(SEQ ID NO: 362)
TAGGATTGGCCGTGTTTTGTAAAGAAATTAAGACAAATGCACCCT
GGTAAATAGAGTGGCAGCATGTTGTTGCAGTGCCTCTGAAAGACA
AGGTCTTGGTTTGTTTGAAAATTTGAGATAAATTTGGAAGCAGTT
AACTCATTTGATAGAAGAAGTACTTGATTCTTCTGAGAATTGTTC
AGATTTGTATCTTGCCATTCTAATATTTTTTCTTTTAAAAATGTA
TTGGGTGGGGCAGAATTTGCTTAAATATTTAAATAGTTGGTTATT
GGGATTTTTGTGAACTTCTTCTTTAAGTAGCAAGTAGTGTGGTCT
TTAGGATTTTTAAAGGAGATACATGTGAAGTGCAAAATTCTTGTA
TGAGGTGGAATATGAAAATAGAATTTATTTTATTTACTAAAACCA
GATCCTTCTTTGGGATTGCAGGTGAGAGTGAGTGAGTGCGTGTGT
GTGTTTGTTTGTGTCTGTGGTGTGTCTGTGTGTAGGATTCTTTAG
GAAT

In some embodiments, the intronic domain that promotes trans-splicing primarily in the brain comprises or consists of sequences from the gene CNOT1. In some embodiments, the sequences from gene CNOT1 comprise or consist of:

(SEQ ID NO: 363)
GTTGGTTTTGAGCTCCTCACCTCAAGTAATCTGCCTGCTTCGGCC
TCCCAAAGTGCTGGGATTACAGGTGTGAGCCACCACGCCCTGCCA
TAGTCTCCATATTTCACAGTTTTGTGTCTGTGCACTTGTGCAGTT
GTTCCTTGGTCCCTTGCTATTCTTGGAGGATTGGTTCTAGGACCC
ACTGGGACACCAAATTTTAAGTATGCTTAAGTCCTGTATGTAAAG
TGGCATATAACCTTTGCATATCCCCTTAAATGACATCTGTAGTGC
TTATGATACCTAATGTAGTGTAATGCTATTATGGAAATAACTATT
TTAAACTATATTAATAACTTTTTAAAATTTGTGTTTTTTATTGTT
GTATTATTATTTATTTGTATTTATTTATTTATTTTTGAGACAGAG
TCTCACTCTGTCACCCAGGCTGGAGTGCAGTGGCGCAATCTCAGC
TCACTGCAACTTCCGCCTCCTGGGGTCCAGTGATTCTCCTGCCTC
AGCC

In some embodiments, the intronic domain that promotes trans-splicing primarily in the brain comprises or consists of sequences from the gene CNOT1. In some embodiments, the sequences from gene CNOT1 comprise or consist of:

(SEQ ID NO: 364)
AGTTCCTTTGTGATTACCATTATGGGTTCTGTGATGTGATCCCAC
CTAATTGTATCCAGTTAAGAAATTTGATCCTGAGTGCCTTTCCAA
GAAACATGAGGCTCCCCGACCCATTCACTCCTAATCTAAAGGTAA
GGTTCCAACTAGTTAAAGGAAAACTTATGTCTTAATATTTCAGTC
CTTTCTCTGATAAGTAGACTGTTCAGCACTTTCCTACCCAACATA
ACACGCACACTATTAGTTAAGGGTACTCTTTAAAGGAAATAATGT
TATTAAGCCTGATAGAAGGTATGATATTGTTGCTATTGCTGTTGC
TGGTGAATATTGGTTCACAGCATTTTTCTAGTTGCTAGTTTTCAT
ATTAATTTTGATATTAAATTTAGCTCTATTTACAGAACCACAGCT
TAGGTGTTGGGGCAGTCATTTGCTGGGTGTGCCCCACATGGCAAA
GAATTGCAGAACAAACAGACCTTTGTAATCTTTTTCTTTTTTTGA
GGCA

In some embodiments, the intronic domain that promotes trans-splicing primarily in the brain comprises or consists of sequences from the gene CHTF8. In some embodiments, the sequences from gene CHTF8 comprise or consist of:

(SEQ ID NO: 365)
GTACGTGCGGGAAGGGGCTCTTCCCGGGGTTGGGAGGGAGCCCAG
CCCGCTATGCTTGCGGGAGCGGAGTAGAACTGGAACCTAGGAAGT
AGCGGGAAGAGCAGAAAGGGGAGTTCGGGGTGGGGGTTAGGACCG
TGAGGTTTTGTTCCAGGTAACATTTGTAATCGTGGAGGGGGGAGG
CTTCTGGGAGAGGTAATTGGAAGGGTGGTTGGAGAGTCATTCCAG
GTTCTTTTGTGGGAGGGAAGCCGAGAGACGAGTGGGGTGGGGTCC
TGGAGGAGTGGAGTACTTGAGAACCGGGAACAATTTGGAGAACGA
GGGTGGTAGAGAAAGGACGTTTAAAGAGTAATCTTTGGGAAGAGG
GTGAACGGTTTGGAAGACCACTCCAGTGGGTGGTGTGGAGGAAGG
CGGTTTAAAGAATAGTTTCCCTGGGAGGCAGGAAAAGGGGTTAAA
ACAGCTTGTAATTTGGAGGGGGGTGGAGAAGAGGGGGGAAGCGGA
TTGA

In some embodiments, the intronic domain that promotes trans-splicing primarily in the brain comprises or consists of sequences from the gene CHTF8. In some embodiments, the sequences from gene CHTF8 comprise or consist of:

(SEQ ID NO: 366)
CCCGGTGCTCGCCTCTCTCCGCCCCGGCCCCACGTGCGCGCGCGC
TCTCAGGCTCTGCCCCGGAAGTGCTCCTGGCGCATAGTCGGAAGT
GCGCAGGCAGTGCTCGCGGCGGCGGCGACGGCGGCGGGAGGTTCG
GTTGTCGCCCGTTGGCCGCGCGGCGCCGCGCTCGGCGGCCGCCAT
TGCAGGTCAGGCCGCGGGCGGGGGGCACGGGAGGCTGCGGGGAGC
GAGGGCCGAGCCGGGATTGGGGGCGAGGGCGCGAGGGAGCGGGAA
GCTAGGGAGCCGGGGCCGGGCGGGGCGCGCGGCTCGGAGAGAAGA
ATGGCGAGCCGAGGGGCGGGGAGCGCGGGCGGGGGAGGGGTGGCC
GGGAAGGGGGAGGGCGGCGGGAGGGCGGCGCGGAGGTGGGGGCAA
GCCCGGAGGGGCCTCCGGGGTGGGAGGGCCAGGAGTGTGGGGGAA
GGGAGCGGTGTGGAGACGGAAGGCGGAGGGAAGGAAGGGAGCGAG
CGGG

In some embodiments, the intronic domain that promotes trans-splicing primarily in the brain comprises or consists of sequences from the gene CALB2. In some embodiments, the sequences from gene CALB2 comprise or consist of:

(SEQ ID NO: 367)
GACCACTGGATTTAGGGCCCACCCCAATCCAGTATGACTCATCTT
AACTAATGACATCTGCAATGACCCTGTTTCCAAATAAGGTCCCAT
TCTGGGGGAGCACTCCAACCCGCTACACCTTCTGAACTCCAGGGA
CCCCAGTTCTGCCTCTGACCAGGTCCCGCCACTTGCTGATGACCT
GTCACACCTCTGCCACCTCGTGCTTGCCCAGACATTAAGGACAGA
TTCGGAAGCTGCCAACTTCTGCTCTCCTCTGTCCTCTCGGACCCT
GTGCTAGTTCCTCCCAGTATTTTCTAGCTGCTGAATGTATGTTGT
TCATCTTGGATCTCTAGGTGCAAATGCCCTGAGGGCTGGACCCAC
ACCCCTAGCTCCACTGGCCTAGACACCTGCCTGTTGGCCAGCTGG
GAGAAGACATTCTCCACGAAGCCCCCGACATGCCCACCCCGTGCC
CCCCTTACTATTTAGTGCTGAGATTGATTTTTTCTCTCTCTTTTT
ACAG

In some embodiments, the intronic domain that promotes trans-splicing primarily in the brain comprises or consists of sequences from the gene CALB2. In some embodiments, the sequences from gene CALB2 comprise or consist of:

(SEQ ID NO: 368)
TAAGCCCAGCCCTGTCTCCGATTGTGTGGGATTTTCCTGACCTAT
GCCTTTGAGCATGCTGTGTCCCAGTTATGTATGCCATAAGCAGGC
CTCATATCGCTGGTCTTGACACAGCATTTTATTTGTAAGGATAAA
AAATAGACTGTCCCCTAAGAGTACCCTATCTCAATATCCTTGGGT
GTCTGGGATTGAGGGGTATTCATGAGTGGACACCAGGGGTTGATG
AATCCTCCTGCAAAGCTGCAAAGCTTTGTCTGCGTATGCGTATAT
TAATATATATGTAGTATTTTTTTGTCCCTAGGGACAGAGTCCATA
GCTTCCCTCAGGTGCTTAGGGGTCATGATTCCAGATTGGATGCCC
AGGAGGTTTCAAGTGCCCTGATTCTAGACCAGAGTTTCTCAGCCT
CAGCGCTGTTGGCATTTGAGGCTGAATAATTTGTTGCAGGGGCTG
CCCTGTGCGCTGTAGGAGACTAGCAGCATCCCGGGATCTACCCAT
GAGA

In some embodiments, the intronic domain that promotes trans-splicing primarily in the brain comprises or consists of sequences from the gene IST1. In some embodiments, the sequences from gene IST1 comprise or consist of:

(SEQ ID NO: 369)
GGTATGTGCCTACACGCCTGGCTAATTTTTTCTTTTTTTTGTATT
TTTTTTAGTAGAGACGAGATTTCACAATGTTGGTCAGGCTGGTCT
TGAACTCCTGACCTCAGGTGATCCACCTGCCTCAGTCTCCCCAAG
TGCTGGGATTACAGGCATGTGCCACCGCGCCTGGCATGGAACCTG
GTTTTATCTTGAAATGTTGCAAACGTTTATAAAGATACAGTATTT
CTAAATTCACATCAACATCTTATGATAAGGGCTAGCTCCTACTTG
TCATATGACTGCCCCGGTACAAGCATTAGATGCAGCCTCCACAGC
AGGGAGCAGATGACTTCCTTCTGGAGGCCCCTTTTTCTTTTTTCC
CTTTTTTTTTTTGAGACGTTTCGCTCTTGTTGCCCAGGCTGGAGG
GCAATCGCCGGATCTCGGCTCACTGTAACTCGAGAATCAAGCGAT
TCTCCTGCCTCAGCCTCCGGAGTAGCTGGGATTACAGGCTTGCGC
CACC

In some embodiments, the intronic domain that promotes trans-splicing primarily in the brain comprises or consists of sequences from the gene IST1. In some embodiments, the sequences from gene IST1 comprise or consist of:

(SEQ ID NO: 370)
TGATACACCTGCCTCGGCCTCCCAAAATGTTGAGATTACAGGCGT
GAGCCACCGCACCCGGCCCTTTTTCTTTTTTTCTGTAGCCTAGGC
TGGAGTGCTGTGACGTGATCACGGTTTACTGCAGCAGCGACCTTC
TGGGCTCAAGCGATCTTCCTGCCTCAGCCTCCCGAGTAGCTGAGA
CCACAAGTGCTCACTGCTGCTCCCCTAATTTTTAACTTTTTTTTT
TTTTTTTTTGTAGCGATGCGGTTTCACTATGGTGTCCAGGCTGGT
CTGCAACTCCCGGGCTCAAGCGATCCTCCCGCCCCGCCTCTCAAA
GTGCTGGGAAGGTCCCTTCCAGGCTTAAGCAGCGATAATGGTTTT
CAAGTTAAAAACAAAGGTAACCAAGGAAAAAGTTTTCTCTGAATA
CCGAGATAAATTAGGGAAATCGCCAGAGACTGTGGTGCTGAGGAA
ACACTACTGAATTATACCACACAGACCGGAGCGATGCTGGTCCAA
AGGG

In some embodiments, the intronic domain that promotes trans-splicing primarily in the brain comprises or consists of sequences from the gene GLG1. In some embodiments, the sequences from gene GLG1 comprise or consist of:

(SEQ ID NO: 371)
GTATGTATTAGATTATTTGCCCATCAGATATTACTGATTCAGAAAGAGA
AACTGCTCCTCCTCCACCCTGGCTCTTTCTAAAGCAGAAGTATTAATAT
ATTTAATTTTCTACAGGATATTCAAATAGAAGCCTTGCTGATGAGAGCC
TGTGAGCCCATAATTCAGAACTTCTGCCACGTAAGTGATGTCTGGAGGA
GCAAGGGTTAAAAACAGAAACAAATTGACAGGGACTCTCCCCTGAGTAT
CTCAGACTCACCTCTGTCTGCAGCATGTATTACTCTTGAGTGTTAGCCA
GCTTTGACAAGTTAAAATTAAAAATGTAAACAGGCCGGGTGTGGTGACC
AATACCTGTAATCCCAGCACTTTGAAGCCAAGGAGGAGCACTTGAGCCC
AGGATTTCAAGACCAGCCTGGGCAACATGGCAAAACTCTGTCTCTACAA
AAAATAGAAAAAATTAGCTGGGCGTGGTGGTACACACCTGTAGTTCCAG
TTACCTAGG

In some embodiments, the intronic domain that promotes trans-splicing primarily in the brain comprises or consists of sequences from the gene GLG1. In some embodiments, the sequences from gene GLG1 comprise or consist of:

(SEQ ID NO: 372)
ATTTTGTGGGGCCAGAAATCAGAGAAGTGGGGAACTACCATTTTGTCTT
AAACCTTTTAGTACTCTATTTGAATTGTCATAATGTAAAATTACGTTTC
TCAACCAGTTCTTTTTTGCTTTCTAGTTTTCAGAAGTTCATTGCCTTCT
CTCATTTCGTCGTCATCTCTCACATTATCATTGTCATCATGGACTTATG
AGAGCAGTCTGGGTGTATAGCTCTTTAAAGGAAGGTGTTTTTTCAGGAG
CTAGTGTTGCACGGGTTTCTCTCTTTAAAGGCATTATTAGTATGTGTTC
CCCAGTAACAGTGGTTTGGCATCTGCTTTGAGGGCAGTGCTCTGGCTTA
TGAGCTTATAAACTGAAGATGGATATCAGTTACCCCTGTTTTCATTTTG
AACTACTTAGCATTTTGGGAAAATCTGCAAAGGCTTTGCATACTCTAAT
GTTATTGCTACATGTGTTTTTACCTCACTTTTCTTAATTCTGCCTTCTC
ATTTGTTCA

In some embodiments, the intronic domain that promotes trans-splicing primarily in the brain comprises or consists of sequences from the gene BCAR1. In some embodiments, the sequences from gene BCAR1 comprise or consist of:

(SEQ ID NO: 373)
GTAAGAGCACCTGGGGAGGGGAGGGCTGGCCCTGGACCCACTTCCCCCA
CTAGGCCTCATCCTTTCAGGTCGCTGGGGATCTTACCTGCTGCAGGCAG
CTCCCCCGGGGCAGGCGCGGCAGGGAGGGGGCCTTGGTCCCAGCTGCTG
CGCCCCCCAGCACACACCCAACCTGCCCCTCTGGCCCTGTCCTCCAGAG
TCTCCCCATGCTGTGCTGTGGGGCAGGGGGTGCTAGGCTCTATCTGGTC
CTGGGCCGGATGCAGTCCCCTGCTGGGCCAACCCTTCTGAGGAGGCTAC
AGCTAGGTGGTGTGGGCAGCAGACTCCCAGGCCTCCGACGTGGCCTCTT
GTCCTGTGGGGCTAGGCGCCCCAGTAACGGGGGGTTCCTTGCCTGTGGG
TAGTGCCCTCCAGAGCCAGGGCCTCAGCCCCTCACCCCACCGGTGTCAT
CAGCTCATACCCATGCTCCTCTGTGGAGCCTTCTGGGTCAGCAGGCTGT
AGAGACGTT

In some embodiments, the intronic domain that promotes trans-splicing primarily in the brain comprises or consists of sequences from the gene BCAR1. In some embodiments, the sequences from gene BCAR1 comprise or consist of:

(SEQ ID NO: 374)
CATTTTGCAGAAGGGGGAGACCAGCCACTGAGCTCCAGGGCGGGGGCCC
ACTTTGTCCCCTTCCTCCGACCTGACACCTGCCTCTGGGGCCCTTACCG
GGAGGCTGGGATGTATTCCTGAGTTCGCTTGTCCCCCCGCTTGATGCCT
GATAGTAGGGGAGGAGAGGGGCGGCACCCTGGCCCTGGGGCTGCCTGGG
TTCTGTGCGTGCACCTGGCAGTGGGCCTGTGCTGTGTTTTCTCTGCATT
GTATGAGGCATGTGTGTCAGAGACCGTGTGGGTCTTATGTCAGTGACTG
TGCCTCTGTTCTCTCCTCCTGGGCTCTGGCTGTGTTCCCCTAACCCCCG
GGGGACCTCCACGGCCTGCTAGTGGGGTCTCAGGTGTGCAGCAGGGTGG
CCTGTGATTAGCAGTGGGGCTGAGTCATTCCCCTAAAGAGCTTTTCCCC
CTGGTGGCTGCGTTGCTCTGTTTTTCTAACAGAAACATTTGGTCACTGT
GGCTGCCTA

In some embodiments, the intronic domain that promotes trans-splicing primarily in the brain comprises or consists of sequences from the gene ABR. In some embodiments, the sequences from gene ABR comprise or consist of:

(SEQ ID NO: 375)
GTAGGTACACCGGCTTTTCTTTCTGGGTTTGGGGAGGGGTGACGGGAAG
GCCTGGGCATCGTGGGCCAGGCCCTGCTTGGGTTGGTCTCGAGAGATTG
GGACTGGAGACGTTTGGACCTGGCCGTTCTTGGTGCCTCGTCATCTCGT
CCTGGCCCTGGACCCCTCTCAGGCTCGGGCTTCCCTCCCCACCTGCTTT
GCCAACGCTGCCGCCCCTGTCTCTCCCCGTCTACTAGCCGTCTGCTCCC
CAGGCCTCCCAGACCCCTCCGTGCCTCTTTCAGCTTTCCCCTGTTAGCC
TGTCTCCTCCAGAGGACCCAGGAGAGGACCAGGCTCTCCTCCCCTCTGC
ACCCAGGCCAGGCAGTGGCTGCATGCGTGGCCCGTGGGCTCCAGGTGTT
CAGATGCTGCTCCCGCAAAGCCAGCCTGGGCCTGGTGCTCCCTGTGGGT
GCAATGAGCTGCCCGCTCCAGCCCTCCTCTGTTCACTCTGGAAGGGAAC
AAACTCTGA

In some embodiments, the intronic domain that promotes trans-splicing primarily in the brain comprises or consists of sequences from the gene ABR. In some embodiments, the sequences from gene ABR comprise or consist of:

(SEQ ID NO: 376)
CTCCAGAACCTGCCCTGGACCCCTGGAGGGAGTGGGCTCAGTACAAATG
GTACCCAAGGAGGAGGGCCACAGGAAGTGACATCGACCGGCCCCCCCCA
CCTCCTTATTCCCTATAAAAAGCAAAATGGTGACTCAGAGAGGAGAGTT
GGAAGCGTCCTGCCTCTTGCTCCCACTGTTAATTTTTACACTTGGAATT
AGGCAGAAGGCAAAGAAAACAGCAGCTTGAAATACGCGGCTGACATGCT
CTTCATCGGGTAGCAACAGCAGAGCCGCTCCTCCCCGCCTCCCAGCAGC
CCCGGAGCGGGCAGGGCCCGGTGCCTGCTGGCTCTCCCCCGCCCCTCAC
CAGGCCCAGGCCCCCGCACAGCCCCCGCCCGGGCCAGGCGTGGATGGTG
CGGCCGGGGCCATGGCTGCCGGGGGCCGCCGCCGGCGCCCACTACGCTA
CCAGTCCCTGGCAGCTCTGGTGGAGGATTCTCAGTGGCCTTTCTTGTTC
CTTGTCTCA

In some embodiments, the intronic domain that promotes trans-splicing primarily in the brain comprises or consists of sequences from the gene UBB. In some embodiments, the sequences from gene UBB comprise or consist of:

(SEQ ID NO: 377)
CATTGCACTCCAGCCTGGGAAACAAGAGCGAAACTCCGTCTCAAGAAAA
AAAAGGAAAGACCCCCCCTCCTTCTCCCGCCGGAAATACCCTCTTTCAG
GACGGCGCGCCTGTGCGGCGCACGCGCGCTCAGTTACTTAGCAACCTCG
GCGCTAAGCCACCCCAGGTGGAGCCCAGCAACAACAGAGCCACCGCGTC
CCCCACCAATCAGCGCCGACCTCGCCTTCGCAGGCCTAACCAATCAGTG
CCGGCGCTGCAAGGAAGTTTCCAGAGCTTTCGAGGAAGGTTTCTTCAAC
TCAAATTCATCCGCCTGATAATTTTCTTATATTTTCCTAAAGAAGGAAG
AGAAGCGCATAGAGGAGAAGGGAAATAATTTTTTAGGAGCCTTTCTTAC
GGCTATGAGGAATTTGGGGCTCAGTTGAAAAGCCTAAACTGCCTCTCGG
GAGGTTGGGCGCGGCGAACTACTTTCAGCGGCGCACGGAGACGGCGTCT
ACGTGAGGG

In some embodiments, the intronic domain that promotes trans-splicing primarily in the brain comprises or consists of sequences from the gene UBB. In some embodiments, the sequences from gene UBB comprise or consist of:

(SEQ ID NO: 378)
GGCGGTTGGCTTTGTTGGGTGAGCTTGTTTGTGTCCCTGTGGGTGGACG
TGGTTGGTGATTGGCAGGATCCTGGTATCCGCTAACAGGTACTGGCCCG
CAGCCGTAACGACCTTGGGGGGGTGTGAGAGGGGGGAATGGGTGAGGTC
AAGGTGGAGGCTTCTTGGGGTTGGGTGGGCCGCTGAGGGGAGGGCGTGG
GGGAGGGGAGGGCGAGGTGACGCGGCGCTGGGCCTTTCCGGGACAGTGG
GCCTTGTTGACCTGAGGGGGGCGAGGGCGGTTGGCGCGCGCGCGCGTTG
ACGGAAACTAACGGACGCCTAACCGATCGGCGATTCTGTCGAGTTTACT
TCGCGGGGAAGGCGGAAAAGAGGTAGTTTGTGTGGTTTCTGGAAGCCTT
TACTTTGGAATCCCAGTGTGAGAAAGGTGCCCCTTCTTGTGTTTCAATG
GGATTTTTATTTCGCGAGTCTTGTGGGTTTGGTTTTGTTTTCAGTTTGC
CTAACACCG

In some embodiments, the intronic domain that promotes trans-splicing primarily in the brain comprises or consists of sequences from the gene LRRC75A-AS1. In some embodiments, the sequences from gene LRRC75A-AS1 comprise or consist of:

(SEQ ID NO: 379)
CTGGCGTAGCGATACAGAACGTCGTCTAGCGAGGTCGACTCCATGCCCA
GGTCCTGCAAGAGCAAAGGATGCCCAGAGGTCAGGGCTGGCTGCCCACC
CAGCTCGCCCACCATCCCCAAGAGAAGTAGGCACAGACCCCTAGAGGCG
ACTCTGCCTCCCAGAGCCCCGGTGGGGAGCATCTGCAGAACTTCCCTCA
GGGGCTGGGGCAGGCCTCTCCTGTTTGTCTTTGCCTCCAAGTTGAGGAA
GAGCAGGGACTGGCCTCTGCAGGCTCTGCAGTTTGTGCAGGTCCTGGCT
TGTGGCCCTCTCCTAGGACAGCCCCTGACCTTTGTCTCTCTGGTTTTCC
AGAGGAGGAAGCCAAGGGTGGCCTGCCTCGGTGCAAGCAGCTGCTTCTC
TATGGTGGCAAAATTTGGAATTAACTGCCTTGTGACAGGATGGTTTAAT
GATATTTATTTGTGTTTTCTTCCTGCTTCTGACGTTGTTTTTACTGCTC
CTTCTGTAG

In some embodiments, the intronic domain that promotes trans-splicing primarily in the brain comprises or consists of sequences from the gene LRRC75A-AS1. In some embodiments, the sequences from gene LRRC75A-AS1 comprise or consist of:

(SEQ ID NO: 380)
TACTGAGTGGAATCTTCCCGATGGGAGGGGAGCAGCTTGGAGGCGAGTG
CTACTAAGTGTGGTGGGGCCGCGGCAGAAACCGCTGGAGCAGAGCTTCC
CTGGGTCTGAGGAAGGAATTGGGTTTGGGGGCTCAGGACACACCACCCC
AGAATATGGTACCCTGGCATCTGAGGAGGCAGCTGACACAGGAAGGTCT
CTCTGACTGCTGTTCTCCCCCAAAGCAGGCCCTGAAGAATCCTGTGACC
TTCATCTAAAGTAGGTCATGAGACCCCTGAGCTTAAAACGGAGTGATCG
GCTTTGGAGAAGTAAGGAGACATGACGTGCCTGCCCTGGGGTGGGGACT
AAACTTCGTGCCACTCCACGTGAAAGGGGGAGGTACGCCTGCTCCCTTT
CCTCAAGCCCCCCCCTCCCAATTTCTGCCATCCTTGGGTATGAGCTACA
CGACAGTCCCTGACCCTAGCTTGGTGCCCTGCACAGACCAGGCAATGCT
GACATTCCC

In some embodiments, the intronic domain that promotes trans-splicing primarily in the brain comprises or consists of sequences from the gene ALDOC. In some embodiments, the sequences from gene ALDOC comprise or consist of:

(SEQ ID NO: 381)
GTAAGGCCAGGCAAGGCGGGAGCCACTCAACATCTCCGCTTCATTTCCT
TGGCTCTTCCCCTGCCATCCTCGTCCTGCTACCTGGGACTCCGGGATGT
GCCCTTTCGACCCTTTCCTAACATCTTTGCTCCTTTCCGCGTACTTGAA
ACCCCATGGCTCAACCTCTTCTGTTCTATCCCTCTTCAGCTCTCCCAGC
TGGACCTCAGCTTTTCCAATCCCAAATCCTCCTCTACTTGTTGGATTTT
TCCTTGGAGTCTGTCTCTCCTACCCAGAGCATTTCCTTCTCAGCCTGCT
CCCTCTCCTCCTAGGCTAGGTCCTCTCTCCAGTTCTCCCGCCTTCTCTG
CCCCCCGGTCTAGGTCTCTCCCGCTGACTGGTTAGCCTGCATCACCACT
AGCTCCCCTCAGTCTCATCTCTCTCTCAGGCTCTCTACTCCTTTCAGCC
TTGGTCCTCTGCCCTCCCGTCTGGGTGTCTGAGTTGTGGGGAATTGAAG
TGCTTCCTA

In some embodiments, the intronic domain that promotes trans-splicing primarily in the brain comprises or consists of sequences from the gene ALDOC. In some embodiments, the sequences from gene ALDOC comprise or consist of:

(SEQ ID NO: 382)
CCTGCCCTTCCTCCCTTCCTCTGCTTTCACCAAAACTCTGGGCACTGGA
CTGCTTGTTCCCTGGCTCACCGCCTGGAGGAAACAGAAAGGCTTCATTC
TCTTACTTTCCCCAGCCCCCTCGGGGGTATACCCTGGCAAATATGGGGG
TGAGGACAGAGAAGCTCAGTTCATTACTTCCTGCCCTGAACTTGGGCAC
CTTGAAGATGCTTCTGCCCCACCTACTCAACCTGTTGTTACTGTTCTGG
TGCCTCCTCTTCACCAGACTAAGCTTAATCCCCAGCATCCAGCAAGTGC
CCGGCACGTGGTAAAGGGATTGTGGCAGAAATGAACGCAAGGTCATGGC
CAGGCTCCCCGAGGTGGGAGGGCAGTCCCTAACAGCCACGGATGCCTGG
CGGGGCGCTCACTCCACCTCGCCGCCCCGCCTCTGAGGGCGTGGTCTTG
CCCCGGACACCAGTCCTGGGGAGGGGGTGTGGTCAGGGCGGGGCATGCA
GGCCACGCC

In some embodiments, the intronic domain that promotes trans-splicing primarily in the brain comprises or consists of sequences from the gene GIT1. In some embodiments, the sequences from gene GIT1 comprise or consist of:

(SEQ ID NO: 383)
GTATATGGTGCCTGCCGGGGCAGCAGGCCTGAAGGTGGGGTAGGGCGGG
GAGGCATTTGGTGTGCAGCATCTGGGGTGCCATCTCCCCGGGATCCCTG
CATGTTGGGGAGGTTGCTGGCATGGGCCTGGCCACCTCTCAGGTCTCTC
CTCTCCTCCCAATTCCCATCTCCCTCTGCCCTCAGCCTTGACTTATCCG
AATTGGCCAAAGCTGCTAAGAAGAAGCTGCAGGCGGTAAGTCTGCTCCA
ACAGTGCCCTGACACTTGAAGCTGGCCCCTCTCTGATCCCCCACCCTCT
GCTCTTGCTTCCTCTGCCTCTGACTCGATGACCTTTTCCAAGTCTTTGA
CCTGTCCCCTCACTGGCCTCAGCAGTCCCCTCTCCCTGACCTCCCTACA
AGGGGATGGACTGGTCCCTTCTCTCCCACCTTGGAGTTTGGAACATGAT
CCCTTTCCACTTTCCCATTCTGAGACTGTAGGGGCTGGGCAGCACAAGG
AAGGCCACC

In some embodiments, the intronic domain that promotes trans-splicing primarily in the brain comprises or consists of sequences from the gene GIT1. In some embodiments, the sequences from gene GIT1 comprise or consist of:

(SEQ ID NO: 384)
CAGGTGTGAGCCACTGTGCCCGTCCCTAGGGTATTCTGATGAAGTATGT
TTGGAGTGTGCCATATGCACCTGTTATCACTGCAGTGCCTTAAGGCAAG
GGGTAGTGCAGCCAGGTGTATCCAAAAAATCACCCTGGCTGCTGGGTGG
AACAGGATTGTAGGGACAAAACTAGAAGGAGGGAGATTAAGAAACTTAC
AAAGCCCAGGCAACAGGTGATTGAGGAACCTGGGGAGCTTCACTAGGAC
TGGAGGGTGAGGGAGGCTTCAGGTGACTGCCAGGTGGCTGACACTGGTG
GTGGACAAACAGGTCACTGAGAGGAAGAGCAAGCCAGAGGAGGAGGGGT
GGGGGACGCGGTGAGATGAATTCAGCTTTGTCCCTTCTGGGCCGGGTGT
GCGTGGAGATGGGTCTGGGCCTGATGTGAAAAGGCGTGTGCTGCCCTGC
TCCTGGGTAACTGCCGCCTCTGTCCTGTTCTTCCCTCTGCTGCCTTCCT
CTGGACTTA

In some embodiments, the intronic domain that promotes trans-splicing primarily in the brain comprises or consists of sequences from the gene ATP6V0A1. In some embodiments, the sequences from gene ATP6V0A1 comprise or consist of:

(SEQ ID NO: 385)
TCTTGCTGCAGATATTTTGTTTGAGCTTTTTTGGTGTCATTTGCCTACT
TTTTACAGGGAGAGAGAGAATTAGGCCAAACTAGACTTTTCCTTAAGAT
ACCACCTCCAAGCCCACTCTCCTTGCCTGACCCCTTGTAGCACAAAGCC
TCTGAACTGAACTATTAAATCTCATCTACTCATTATGTTCTTTTTAAAG
CAAATAAAATAGGAGTTGAGGACTCAAGTTGGACTTTTCAGTCTGTCTG
ACCACTTTCACTATATATGATTTTTTAAAAAAGAAATCACAAGGGTTGT
GGATGCCTTTAAAAAATATGTATCTTAATGCTCTAAATTCCTGCTGATT
TCGCTTTGTGTATATGCTGAGGAGGCCTGCCTCCAACGTGCTAGCCCAT
TTAACATGAACAGTCCTCCCCAAGAAGCATTCAGTAGCCTTGTGCTCCT
AACTTGTGACCTTGTGTGTGGCGTGGCTCCCCACTAAGTGTAAATTTGT
GTTTTCAAG

In some embodiments, the intronic domain that promotes trans-splicing primarily in the brain comprises or consists of sequences from the gene ATP6V0A1. In some embodiments, the sequences from gene ATP6V0A1 comprise or consist of:

(SEQ ID NO: 386)
TAAGATGAAAGCTGGCGTTGCCTTCGTGTTTATCCGGGCTTCTCTCTTCC
CATCCTTGTGATCACTCTGCTGACCCTGCCACACCCTGGGGCCATACACA
TGACTCCTGTGCCTCTGCATCTCACTGGCCATTTCAAACCAGTTCGTGAG
CCTGTGTTCTAAAAAGCTCATCTCCATTAACTGCATCTCATGTCGGTCAC
CTCTGTCTTTGTTTCATTGACTTTGCTGATTTAACACCTCTGAGAATGGC
TTTGAAAGGAGCTACAAGTAATGTCTTCACTGGCATTCTTGAAGTGACTC
CTCTTGGGGGTTAAGTCCCATTGGCCTCTGCCTTCCTCTCCTGTGATGTT
GTGGTTGATCTACTCCCAGCCACCGGGAGTAGCCGTTTCCTCCCTGCTCC
TCCTAGTGCTTGTGAGCAAACCTTACCCTTCCTCCCTGTCTGAGCCAGCC
TCTTCCTGCTGCCCTCCTCAGCTTGATGATGCTTCAACTTAGAAGTGGT

In some embodiments, the intronic domain that promotes trans-splicing primarily in the brain comprises or consists of sequences from the gene PSME3. In some embodiments, the sequences from gene PSME3 comprise or consist of:

(SEQ ID NO: 387)
CCTACCCGACACTTGACCCCGGCAGTGTGAACCCCGCTGGGAGCGTCCAA
AGCCATGCCTCATCGCGGCTGCGCAGCGCACGTGCACAGCCTCGCAGGAG
TCTCGGCGATTGGCTGTGACGCAGTTTCCGGCGTGAGCGGCGAAAGCCGG
GAGGGCGAGCGAGAGAGCAAGCAGGCAGCAGGCTGCCGGCGGGCGGGCGG
ACGGCACAGAGGGAGGGAGCGAGCGAGCAGTGAGTAAGCCAGCAAGGGCG
GTCGGGTCCCGAGGTCAGCCGAGATTTCTCAGGTCCCTCCGGCCCCCTCC
CTGGAGTCCACAGCGCCTCCGGTGTCCAGAGGATCGGACACGGCCCGGCC
CGGCCATGGCCTCGTTGCTGAAGGTGGATCAGGAAGTGAAGCTCAAGGTA
GCGGCACCGGTCCGGCCTTTTTGCCCCTCGCTTTGATCCCCCAGCAGTCT
GACCGGCTTCCTACTCCCCATGGCGTCTTTGTCTCCCTGGGGATACCAG

In some embodiments, the intronic domain that promotes trans-splicing primarily in the brain comprises or consists of sequences from the gene PSME3. In some embodiments, the sequences from gene PSME3 comprise or consist of:

(SEQ ID NO: 388)
TAATCCCCCAGCCCAACAACGTCCCTGGACACTGCCCCTGCCCTTGGCGG
CTGGCCTGCCTAGTATCATAGACTAGGTCTGGGTGGGGGTTGGCACGTTT
GTGAGCTCACATCTTCCCTAACTACCTGTCTTTTGTCTGCTTATCAAATT
CAGACAGCTTCAGACACAGGAGGAAGGGAGGGAAGGGGGAGGACAGAGGA
GACAGGCAGGTGCTGTCCTCAAAGCCCTGCGTTCTTCTGAGATGGAAAAA
TGGATCCTCAAAAAAATAAAGTATTTGCAGTCTGGGGGCCTCTCAGCTTC
TTATTACAGTTACAAGGTGAGGGAAAGACAGTTGGATTGGGATTCTCCAT
CCTGCTCTTACCTCTGTCCCCAGGTACTGAATTGCTCTCTTTGTTAATTT
TAGGTTGATTCTTTCAGGGAGCGGATCACAAGTGAGGTGAGTGAAATAAA
TAGAAAAATGGTTGTTGGTTGAGAATTTCCCAAAGAGGAGATACCTGGA

In some embodiments, the intronic domain that promotes trans-splicing primarily in the brain comprises or consists of sequences from the gene GFAP. In some embodiments, the sequences from gene GFAP comprise or consist of:

(SEQ ID NO: 389)
GTAGCCCTGCCTGTGGGCCAGCCTGCCTCCTCCAGGCAGCCCTTCTTGCT
CTGTACAAGAGAATGGGGGAAGGAGGGTGGAGGAAGCTGGGAGAATATCC
TCTCCAGGTGACTGACTTTCACCAGAGAAGTGAGATGGCTTGCCCAGGGT
CACACAGGCACTATTGCTTTCAAAAGTACCCATTATTATTACTGCTACTA
TTGCTATTATTAGTAGTAGCTGATACTGTTCTGTTCTAAGCCCTGTGCTT
TTCATGGATTACCTTACTATGAAGTAGGTGCTGTTCTTTGACAGATGAGA
GAGCCGAAGTCCAGAGAGGTTAACTAATTTGCTCAAAGTCACACAGCTCC
TAAGTGGTAGAGTTGGATTTCCAACTCTAGCACATCTCACTTGGGAGCCT
GCATTCCATGCCTCTGCCAGTCTGTTTTGGGTTACTGAGCCTCTACCATA
TGCCAGGCACCCCCCAGGACACAGAGAGGTGTCTGACCTGGAAGGTGTG

In some embodiments, the intronic domain that promotes trans-splicing primarily in the brain comprises or consists of sequences from the gene GFAP. In some embodiments, the sequences from gene GFAP comprise or consist of:

(SEQ ID NO: 390)
CTGCTCCCCACTGCCCCAGAAGGAGGGGTAAGGCAAGGCTGAGGAATGGG
GAGAAGGAGCCTGGGGCCCCAGTGGCCCTCTACTCCTCACCCCTCCTGCC
ACTCCAGGAAGCAGATGAAGCCACCCTGGCCCGTCTGGATCTGGAGAGGA
AGATTGAGTCGCTGGAGGAGGAGATCCGGTTCTTGAGGAAGATCCACGAG
GAGGTGAGGCCAGGGCAGAGGGAGGAAAGCTCAACACAGAGATTGCAACT
GAGAGAGGGAGAGACAGAGACAGAGAAGCTGAGACTGAGAGAGACACTCA
GAGAGAGGAGAGAAACAGAGATTGGCAGGTAGAGACAGGAAATGAAAGAC
AAACAGAAAGACACTAAGGGAGACGGAAAGAGACAAGAGAGCATTCGAAC
TCCTGGCCGAGGGGCAGGCAGAAGAGGACACAATGGCGTGGGGAACCCCA
GCTGTGGCCTGTACCCTGAGCCCTGGTACCGCTTCTCTCACCCTGGTCA

In some embodiments, the intronic domain that promotes trans-splicing primarily in the brain comprises or consists of sequences from the gene CDK5RAP3. In some embodiments, the sequences from gene CDK5RAP3 comprise or consist of:

(SEQ ID NO: 391)
TCCTCATATCTTGTGTCCCAGCTCAGGCTATAATCCCTCGATAACAACCT
CCTTCCAGCCTCCTTGTCCCCAGTCTTATCTCCCATGCATCCATCATCCC
AGCAGCCTATCTGTACAGGTTCCTCCCTGCCTCAAACCCCTTCTGTGATT
CCTCTGTCTGAAGGGATGAAGTCCAAGTTCCTTAATAAGTCTCCCTTTGA
TATGGCCTTTCCACCCTTACAGCCTCTCCTCTAGCCACTCGCTGCCTCAA
ACCATCTACGGTATAGAGGTTGGCCTGCCCGCGGAGCCGACTTCATCTTG
AAATTCTTCATCATTTTGTAACAAGCAGCTGTGCATTTCCATTTTGCACT
GGACCTCACAAATTACGTTGCCCGTCCTGCCTGCCTGTTGTTGCTTGCAC
CCCTCTTGCAGTTTGTGGCCACCTAAGCTTTGTCTGTGTAGTCTCCTCCA
CCTAAAACTCCTCTTCCCACATCACCCAGCTATCTTCAGCTCTTCGTTT

In some embodiments, the intronic domain that promotes trans-splicing primarily in the brain comprises or consists of sequences from the gene CDK5RAP3. In some embodiments, the sequences from gene CDK5RAP3 comprise or consist of:

(SEQ ID NO: 392)
TATTTGCAACGGCCTCCCAGATCGGCGCTAGGACCCCGCCAGCAGACGTC
TCAATCTGCGTGGAGAAGACCCAGTGCCCGCCAGGGGAAGCGGCTGCCAG
GCTGGGCTCTCGAGCGCCGCCTGTCGCAATCCCACGGCCGCTGCAGCGCA
CCCCCTCCCGTCCCCTGCCCTGAGCCCGCCTCGCGTCTCCAATTGGCTGT
CTCCATTCTCCCGCCCCTCCCGAGCTCAGCCATTCGGAGCCTGGGGTGGG
CGGGGCTTGAGGCCTGAGTGGAGCGTAAACCCTGATTGGCTGTACGGAAG
GCGGCGACGTGGCCGAAGGATGCCCGTTTGTGTCTAAACGGAGGCTCGGC
CACAACGCCACTGGATTGGTGGTAGGGCGGGGCGGGCCACAGTCTCCAGC
CTGAAGCGGAAGTGGAGGAAAGATGGAGGTGTGGGGACAGGAGCTGGGTG
TGCTGGGGACTGGCCGCGGACCCCTAACCTGTGTCTCCGGTCTCCCTCC

In some embodiments, the intronic domain that promotes trans-splicing primarily in the brain comprises or consists of sequences from the gene CBX1. In some embodiments, the sequences from gene CBX1 comprise or consist of:

(SEQ ID NO: 393)
GTGTGCTGATTGGAGGAGGAGATGTGTGGAATAAATTGGGAAAGGGGACA
CGGAGGGGGAGGGGCAGGCGTTTGCTGGGCCTGACTCTGGAAGGGGAGGA
GCTGTTAGGATAAGTAGGGCGGAGATCCCGAGAATATTTCTATAAGGTAT
GTAAGAAAAAAGGGGAAAGCAGGCCGCCCTTGCATCCTGATGGGTCCTTA
CAGTGGGGAGGCTGTAAGAATAATGTGCGAAGAAAGGATGGAATACGCTT
TTTTCAGTTTTCCAGGTTTCCTACTACCTTCACTCCATGATTCCTTTGGG
GAGTTAAAAAAGTCCAAGTGCAGTTCTTGCTTTGATTCAAAATTAGTTCC
GTACGTTTTTGTGTGGGATCGTAACTGGTCTCACAAAGGTGATGAAATTG
TAAAAATATGTAGATGAGTTAATAGTTGAGGAACGGCGTGAGGTAGTGAG
AGATTGTGAGATCGTGTGAGGTGCGAGAAATTGGGAGACATAGGAAAAT

In some embodiments, the intronic domain that promotes trans-splicing primarily in the brain comprises or consists of sequences from the gene CBX1. In some embodiments, the sequences from gene CBX1 comprise or consist of:

(SEQ ID NO: 394)
GATGACAGGCGTGAGCGACTGCCCCTGGCCCATATGTTCTAATATTCTAG
GTTACAACGCTCTACGCTGCAATCTCTCCACCGTTACAGTTTAAAAACTA
TAAGTCCCTACCACCCTCGCCTACTGTGCGTGCGCGGCGCGCGGCGCTTC
GGGCCTTGTAGTCTTGGACAGACCCCCGAGGCCCATGGTGCCGTGCGGCG
GGTCGTTGCGCCTGCGCGGTGCGAGCGGCGGGCAGCGCAGACTGCGAGGC
TCTTTTGTTCGGCTGAGGGGAGGGCCGTTGGCCGGGGCCTGCGGTACGCC
GCTTCAGTGAGGGACGCCACTGCGGCCACCCGGCTTGCTGCCTTCCTGGG
CGCCACTCCCCCAGGCGACCCGACGCGACGCGCCAGCAGCGCAGCACCGA
TTCCTCTCGGGCTCTTGGGCGCTGCTCTGAGGTGAGGAGCCCGCTGGAGG
CGGGAGAGCTGGGGGAGGGGGCGCGGCGGCGGCGGCGGCGGGAGCCCTG

In some embodiments, the intronic domain that promotes trans-splicing primarily in the brain comprises or consists of sequences from the gene PHB. In some embodiments, the sequences from gene PHB comprise or consist of:

(SEQ ID NO: 395)
GTGAGTCCGACTTGTGAGGGAGGGGAGAAGGGGCTGAGCCCGGGACGAGC
CAGGGGTTGCTCAGAGTAAGGGAGGTGTCCATGGAGGCAGGGTGAGGAAT
AATCCAGAAGCTATTACAAATGTAAAGGGCCGGGTGTCCCAGCCTCAGAG
AAGGAAGATTTAAATGCACTGGACGAGATCAGGGTAGTCTCAGGAGTTGA
GGTCTGGGAAGTAGGGAGGGAGGATTTGAGACTGGAGCGGGCAACGACGG
TGGGGCGGAGCGTTAGAAAGTTACATGCTGGCGTGATTTCTAGTTAGGTC
AACTGTGCTTATGCCCACCCCGCCTCAGCCCCACCCTCCCAGTTATTCCA
GAGCTCACTGTCCCTGTGCAGCTAGTTAGAGCCTTTCTCCCAAATGGGTT
CTTCAGTTATCTTGGCCCCAGGATGTCATCCAGCTCCTGCTTCCATAAGA
AGCATGTCGTTCTTAATACACGATGTTGACAAGCAGTATGGTGAGGAGG

In some embodiments, the intronic domain that promotes trans-splicing primarily in the brain comprises or consists of sequences from the gene PHB. In some embodiments, the sequences from gene PHB comprise or consist of:

(SEQ ID NO: 396)
AAGACGGCCCTTCGTTTCCCCCAAAGGGTCTCAGCCTGCCGGTGATTTCA
CCACGACTTCCTCCAGAGTTCCACCTGTCCTCTTCATCAGCCATGAACTT
GCCCCTACTAATTTTTGCCACCAGCTGGACGCCCACCTCCTCTTTCCCGG
AGCCCTAAGGCTCTCCTTGCGGCAGTCTTAGCCTCTGTACAGGATAGGCA
CGTGCATTTAGCCCCAGAAAACTACAGCCCCCAAAAGGCTTTGCGGCAAC
ATCCTCGCGTTGATCACCAAAGAGAAAACGCTTTTCTCCGCGGGCCCGCC
GCTGCATAGCCTTTTGGGAGTTGTAGTTCATCTGCTAAGAGCCGCAGAAC
CAGGGTGAGGTTCTAAGCCACCCTTCCCAGAACTCACAGCGCTCTTCCAC
ACCAATAGGAAGCGTGGATCTTGGAAAGCGGGCGGGGCAGAGGAGCTCAT
GCGCAGTATGTGTGGTTGGGGAATTCATGTGGAGGTCAGAGTGGAAGCA

In some embodiments, the intronic domain that promotes trans-splicing primarily in the brain comprises or consists of sequences from the gene CLTC. In some embodiments, the sequences from gene CLTC comprise or consist of:

(SEQ ID NO: 397)
ATTACCTCAAATCCCTTGAACCATGCATGCTACCTTGCAGATGTTGCTCT
TTCCTTTAACTTCATCTGTATTTGTTCTGCCTCTTATTTGAAACAGTTGA
ATGGCATGTTTGTTTGAAGAAGAGAAGGTCCAATACTTGGCTATTAATAG
TCTCTCTTTAATTAACATAATGTAGATGTTATTGTGACCTTGAAGCATTA
ATGTTAAGTGAGGATTTGTGTTGATGAGCATATATGAATGTCAAGTCTTT
GGAATCCACTTGGTAACATGGTTTCTTATTATTTTTTCTAGCTCCAGAAC
CTGGGTATCAACCCAGCAAACATTGGCTTCAGTACCCTGACTATGGAGTC
TGACAAATTCATCTGCATTAGAGAAAAAGTAGGAGAGCAGGCCCAGGTGG
TAATCATTGATATGAATGACCCAAGTAATCCAATTCGAAGACCAATTTCA
GCAGACAGCGCCATCATGAATCCAGCTAGCAAAGTAATTGCACTGAAAG

In some embodiments, the intronic domain that promotes trans-splicing primarily in the brain comprises or consists of sequences from the gene CLTC. In some embodiments, the sequences from gene CLTC comprise or consist of:

(SEQ ID NO: 398)
TGGGTTTTCCTTAAAACTCTTCCTATGTTTTTGTTTTTTTTTGTTTTTTT
TTTGTTTGTTTGTTTGTTTTTGAGACGGAGTTTTACTCTTGTCACCCAGG
CTGGAGTGCAATGGCACGATCTTGGCTCACTGCAATCTCCACCCCCCAGG
TTCAAGCGATTCTCTTGCCTCAGCCTCCCGAGTTGCTGGAATTACAGGTG
CCTGCCACCACAACCAGCTAGTTTTTTTGTATTTTTAGTAGAGATGGGGT
TTCACTATGTTGGCCAGGCTGGTCTCGAACTCCTAACCTCATGATCCGCC
TGCCTCGGCCTCCCAGAGTGCTGAGATTACAGGCGTGAGCCACCACGCCC
AGCCAGCTCTTCCTATGTATTTATGCAAGTACAATAACTAGGGTTTACCT
TTTTAAGTATATTGATTAAGTTTGGTTAAAGAAAATAGATCTGCTAATAC
CGAACTTGATTTAAACCACTTTAATTACTTTTACTTGGAATGTATCTGT

In some embodiments, the intronic domain that promotes trans-splicing primarily in the brain comprises or consists of sequences from the gene AMZ2. In some embodiments, the sequences from gene AMZ2 comprise or consist of:

(SEQ ID NO: 399)
CCGCGGCCCCCAACAAATAAAGAAGGGAAAGTGCTGAGGGTGACGGCCCC
GGGGAGCGCTGCGGCTCTACGTCAACCTGCGGCGGCCGCCGACTCATTTG
GGGCCACGCTGGTTGCATTCGTCACGCCGGCGATGCCTCTCAAACCCGCG
GCCTGCCGAGGACGTTCCCACACGGGAGACCCCAGCGACGCGGGCGCATC
TGTGGCTCTCGAGAACCGGGCCGCGGAGCCGCCGCGAGCGCAAGCGAGGA
ATCGGCGACTGCGGGGGTGGACAGCTGGGGCTTGTAGTCCCCTCGCTACC
CTCTATTCTGGAAGAGGCGGGTCGCGGCCGCTGAACTCCAGCTCTGCGCC
TGCCCAGGCGGCCGCACGCTCAGGGGCGTGGCATGGGTGGGTCGTGAGTT
GGGCGGGGCCCACAGGGCGTGCGCGACGCAGCGGCGCGGCGCGTGGCGTA
AGGGGCGTGGCGCCAGTGGGCGTGGCGTGGCGCAGTGCGAAGGGACGCG

In some embodiments, the intronic domain that promotes trans-splicing primarily in the brain comprises or consists of sequences from the gene AMZ2. In some embodiments, the sequences from gene AMZ2 comprise or consist of:

(SEQ ID NO: 400)
TGAGGGCTGCCGCGGGTGGGTGGTATCGAGGCCTGTCGGGTCAGGGCGGT
TCGCGGGTGCTGTCAGAGCTGGGCCGGGGCCCCTAGGCAGGGTAGCCGGG
TCGTAGAGGCGGGGGCCGGTCGCGGTCGGTGGAGCGGGATGAGGATGTAG
GAGGGGCGGACGTGGCGGAAGCCGCGGGGTCCGCGGGGTCGGTGCCTCTA
GGGAGCCAGGGAGGCCTTTCCCGAGGCTCCTGGGGAAGAAGAGGCGAAGC
GAGAGTCCCTGGGGAACCCCCACTCCACTCCCAGCTGGAGACTGGGTTGT
GTCTGCATGGACCAGAGCCCACAGTGCGAGTTGCTATAGGCAACCAGCCA
GGGTGGCCAGCTCCTTCCCGTTTGCCCGTGATGTTCTGGTTTTGGGACCA
AAGCATCCTAGGCCTCCAGCCCACTGCAGTGACCGAATTCTGCGCCCCCT
GCCCATCTTCTCCCGCAGCTTCCCTAGATTAGGCTTGGGAGGCAAGAGG

In some embodiments, the intronic domain that promotes trans-splicing primarily in the brain comprises or consists of sequences from the gene TTYH2. In some embodiments, the sequences from gene TTYH2 comprise or consist of:

(SEQ ID NO: 401)
GTGTGCGCGTGCGTGCATGTGGGGCTCTCGGGAAGCTCTGTATCT
GGAGGGGTGTGTTATTCCGCACCCTCGCTATACACACATTTTCTA
GACCGTAAGTGCTGTGATGCACTAACCTCTAGGTGCTAGAACCCT
GCACGGCACTGAGTAAGTACTCCATAAATGTCTATCAACTTAGCA
AGTTAGTGAGTGATGCTCTATTGTGGACCCCTGAGGCAGTGGCTC
CTGAAGGAGGACGGGGCCACCGGCAGAGCAGGGCACTCAGAGGGG
TCCCCTGCCCGGGGAGCATGTGCCAGAGGAGGGGGACATCCCTAA
GGAACAGGCCAGAGGACCTGAGCTCTCCCTGGCTGCTCTGGAAGG
CGGAGGCCCCCCAGTTTATCCAGTCCCTAGAGCACCCCAGGCCAG
ATCTGGGGTGCGCCTGGCTTGGAGGCCATGTCCTTGCTGCCGCCT
CTCCTGGGGCCTGCGTCTGCTCCCACCCTGCCGCATGGGCCTTAG
CAAT

In some embodiments, the intronic domain that promotes trans-splicing primarily in the brain comprises or consists of sequences from the gene TTYH2. In some embodiments, the sequences from gene TTYH2 comprise or consist of:

(SEQ ID NO: 402)
CCACCAGTGACTTCTGTGTGGCTCCTGACACCTTCATCCTGAACG
TCACGGAGGGCCAGATCAGCACAGGTAACTACACACTCTCAGGCT
GCTGCTGTGGATGCATAGGTGGCCGGACTCTGTGCCCCTACTTAC
CTCTTTCAGCCCTGTAGCCATCCAACCCCTCCTCAACCCTGAACT
TCAAGTCCAGCTCATGCTCTAGGCCATTTCCTAGAAAGTGCCTCC
CTTGGGAGCTCAGGGACGGGGAGGGAGGTCTGGCTTGGCCACCAA
GGATGATAGAGATCTCATCTGGTCATTTGTGAGCTGCCTAACGTT
ATGCCGTTGCCCTGCAGAGGTGACTCGCTACTACCTGTATTGCAG
CCAGAGTGGAAGCAGCCCCTTCCAGCAGGTATGGCCCCCCGAGGC
CTGCCCTCACCCTGCCCACAGCCGCTCCCCCTTGACCCTAAGCCT
CACCACTGACCAAGATGGCGGAGGTGGCAGGGCCTTGCTTGCCTC
ACTG

In some embodiments, the intronic domain that promotes trans-splicing primarily in the brain comprises or consists of sequences from the gene HID1. In some embodiments, the sequences from gene HID1 comprise or consist of:

(SEQ ID NO: 403)
GTGCGCCACGGGGGCCCCGGCGTCCCCATCCACCTGCCGGGCTGG
GGGCTGGGCAGGGCCGGGGGTACGCGAACTTGGCTCGTCGGGAGA
TGGGGGGACGGGCGGGCCCCGAGCGTAACGCCAAGGCCAGCGCCG
GAACGGGACCCCGCGGCCTGTACCTGGAAGCTCCATCCCCTGGCC
CTAGTCCCCCACCCTCAGCCTCCTCTCCTGGGAACACCCCTCTCC
CCGAAGCCGCTGCTGCCACCTTAGGCTGACGTGTGCCTACTCCCC
GAGCCGGCGACACAGCTGTCCGCCCCCTGTCCCGCCCCGCGGCGT
CCCCAGCTTCGCATCTCCAGCCTCCCGCCGCCCCCACCCACCCAT
CCACCGTCCGCTCCCCTCCCTGCCCCTCATTGCCCGCGGGTCCCG
GACGCACAGTCCCTGTTGACTCAATGCAAACAAAGCCTGGCTGGC
GTCCGCCCGGGTCGGAGAAACAGCTGGTGTGCGCGGCAGCGGCCT
CGCT

In some embodiments, the intronic domain that promotes trans-splicing primarily in the brain comprises or consists of sequences from the gene HID1. In some embodiments, the sequences from gene HID1 comprise or consist of:

(SEQ ID NO: 404)
AGGGGTCGGGACCGCGAGGGACTCTCGCGCTCAGCAAGGCGAGGA
GCTGCTTTCCTGGAGTATGGAAACTGAGTCAGCGCCTCCCTCCCT
CATAACCAAGGGCCTGCGGGACTGTCTGAACACGTGGGTTTGACA
GGTCAGAAAAAGGGAGAACCAAGAGAGTGGATGCTCAGAAAACAT
GAATGGGAAAGACAGACTGAGAAGGAGGAAAGTGGGATGGAGAGG
GCCAGCTGAGACGTGGGCCAGGAGCCGGAGCGGAGATGAAGGGTG
AGGACCGGGGCGGGGGACGAGCTGCGAGGAGGGTTTTGGGCCCTG
GGGACTGCGTGGTAGCAGCGGAGGCGGGTACAATGCTCCGACGCA
GAAGGAAGAGCGTCGAGGCTCCGGGTGGGACCTGAGAAGGCACCC
GGCGTGCGGCGGCGCTGGGTTGGAACCGCCGACTCCGCGGGTCTT
CGGCGGCGGGCGCTGCAGGTGCAGGAGGCGGAGCCACGCGGGGGC
GGGG

In some embodiments, the intronic domain that promotes trans-splicing primarily in the brain comprises or consists of sequences from the gene SRP68. In some embodiments, the sequences from gene SRP68 comprise or consist of:

(SEQ ID NO: 405)
GTAAGACGAAGTTCGCTCTTTTCTTGAACGCCGGTTGCTCCAACC
ACTTAACCCCACTTGTCTGAGAAGGGTGTTGGCATGTTTGTTGGA
TTGCATGGAATTAAAACGGGCGCCGTAGCTCTTCCTTTCTGCTGA
GAATGTGGCTGTGGTATATGTGTGTTTCTTTTTTTTTTTTTTTTT
TTTGAGACAGGGTCTCACCCTGTCACCCAGGCCAAAGTGCAGTGG
CACAATCTCAGCTCACTGCAACCTCCACCTCCTGGTCTCAGTTGG
TCCTCCCACCTTTGCCTCCTGGAATAGCTGGGACCACAGGCATGC
ACCACCACACCTGGCTAATTTTTTTTGCATTTTTTGTAGACATGG
GGTTTTGTCATGTTGCCCAGGCTGGTCTCAAACTCCTGGACTCAA
GTGATCTGCCCACATCTGCCTCCCAAAGTGCTGGGATTACAGGCG
TGAGCCACCGCGTCCGACGCCATGTGTGTTTCTTAAGTAGTGGTG
CCAA

In some embodiments, the intronic domain that promotes trans-splicing primarily in the brain comprises or consists of sequences from the gene SRP68. In some embodiments, the sequences from gene SRP68 comprise or consist of:

(SEQ ID NO: 406)
AGACTGCAGAAAAGCCCATCCTGAAGGTTTCAAGTTTTGTAAAAG
CTGACCTAGGATTGATTGATTCATTTGGACTAATCAGTAACATAT
TACTTCTGGGCTTAAGATTTCTCACTGTCATAGGCACACCTGGGA
AAAGGTCTAGTCAGACCACCAATTTTTGTGCAGGGAGTTTTGCCA
CTTATGTTTAAGAGCACGTTCTGGTTGGTCCAGTGGCTGGCGGAG
AAGGTCGGTGTACAGGTGGCCAGGAATCCTGCCTCTGCGTGTTGA
GAGGAGTTTCTTATTCCACTGTCCTTTCAGGAATCATCTTAGCCT
TGAATATAGATTTATAGAATAAAAGTGTAAATAGCAAAATATATT
GAAGGCTTCCTGTTTGGAGACATTTTGCCCTGCCCAGGGAGAATC
ATGAGAGCTTCTCCTTCAGAGATTAGTGGTCTGAGTCATTGCAGT
GTGGGCGTCGTAAGAAAAACACAACTGACTTAAGTCCCGCCCTTC
AAAT

In some embodiments, the intronic domain that promotes trans-splicing primarily in the brain comprises or consists of sequences from the gene EXOC7. In some embodiments, the sequences from gene EXOC7 comprise or consist of:

(SEQ ID NO: 407)
GTAAGCAGTGCGGTTCTGCAGCCCCCTCCTGTCCCTCTGAAGCTT
GGTGGGTGAGGTTCCCTGGGGACTGACTCTCAGTACCTACTGGGG
AGCCTGAGTCAGAGGGGACATGAGTTTTCTCGGGGTGCTCTCAAC
TCTCTCTGCTCCCTTCAAGGCCACTCTGTCTCTGAGCTTATCCGC
AGCACCTGCCCAGGCTAGAGCCCTGGATACACCCCCTAGGCTCGC
CCGGCCTCCCTGCCTTGCTGTCCCCACTCGGCCTTCCCCACTGTG
CAGGCCGCAGCTTCGTCTGTTCAGTAGGGTCTGGTAGAGGGGATG
TTCGAATGTAGGGGAGAAGGTGGAGAGGCTGTGGGGAGCCCATTC
TAAGACTCTGCTGTTGACAAAAGGAAAAAGGGGGCCTTGTAGAAA
CCACCCAGGGTGGGTGGGGGCAGGAGGGGACAGGGCGCTGGCATC
TCACTCTTTTCCTGCCAGCTTTTCTGCTGACATGGGCCAGCTCCA
TCCC

In some embodiments, the intronic domain that promotes trans-splicing primarily in the brain comprises or consists of sequences from the gene EXOC7. In some embodiments, the sequences from gene EXOC7 comprise or consist of:

(SEQ ID NO: 408)
TGCTGGTTGGTTGGAAGGTGTCCTTTCTGCCCCTGCCCCCCTCCC
AAATCCTTCTCTCGTGTCTGCCTGCTCTCCTAATCAGGCTCGCTC
AGCTGGCCTTCAGAGGGTAGCAAGTGTCTCAGGGCCCGCTGGGCG
TCTTCCTGCTGGCCCTGCCCTGGGACTGGAGCTCAGGGGGTTGGC
CTGTAAGCAAGAGCACCTTTCTGGGTCATTCCAAGCACTGAGGTC
CCTGGGCCCTCTCCTCCCGTCCCATTTTGGGGGCAAGGAGCCTGT
GGGCAGCCTCCAGCCTGCAGTTCTCAGCTCTCTACTTTCCAGGGT
GTTCCCTGGTCCTCAGAGGGTCCCGCCTGCTGTGTCTTGCTATCT
GTGTTTTGGCTGTCGTGGGGAGAGGTCTGCTTAATTCGGGTTGAC
ATGCCGTTTCTAACCTGTTTTTTGCACTTTGCATCTTTCTCCCTC
CTTTGTCTCTCCCTCTGTCCCCTCTGTGTGCACTGCCTTTCACTC
CGTA

In some embodiments, the intronic domain that promotes trans-splicing primarily in the brain comprises or consists of sequences from the gene SEPT9. In some embodiments, the sequences from gene SEPT9 comprise or consist of:

(SEQ ID NO: 409)
CAGTCTTCTCCCAGACACTCCCCCGGCTCTGGCCTGGCGCCCGGA
GACCAGTAGCAGCAGCTCCCCACGGCCGTTCACTGTGAGGGTGGG
GAGGGCCGTGGGACCAGGAGGCCTTAGGGGGAAAGTTGGCCATGA
GGAGTGGGAAGCGACGGGTCAGATGCCAGTGACTGCTGGCTGGGG
AGCCGCTGGACGTGGCTGGCCTGTTTGGCCAGTGTGGCGGGTGCC
CCGGGGCTTCAGTCACCCCCAGCCACGTGTGGGGGTGCGGGATGG
GAAAGGTAAGAGTGTGTGGAGCCCAGTCAGCACTCAACATGAGGT
CCCGTGGGGGCTGCCTGAGGGCGTAGCTTCCAGAATTCAGCCCCT
GGCCCTCAGAAGGTGTCACCAAAGGCTTCTTTCCATTTGAGTGAA
TCCCTCCCAGCCCCCAGCAAGCCCTCGGAACGAGCCCACTCCCAG
GCGCTCTCTCCTAGTGTGGGAGTGGCCACGCCCCTGCTGGGAGTG
ACTC

In some embodiments, the intronic domain that promotes trans-splicing primarily in the brain comprises or consists of sequences from the gene SEPT9. In some embodiments, the sequences from gene SEPT9 comprise or consist of:

(SEQ ID NO: 410)
TGAGCTGCGCCCCCATCCCCTGCCCCTCCTCTCCTGCTCCTTCTC
CCTTCCATGGTCCCAGCCAGCAAGCACCTGGGGTAGAGGGGACAA
ACCCAGGTGGCTGTGTTCCAGCCCTGGCTGCAGGTCTGAATGGCT
TTCTGGGGTGGCTGGCCATGCTCCCTGAGAGCCCAGCTGTGGCGA
TGTCTGAGCAGGTAGGTGGGGGAGCACCTAGGAAGCAGGGGTGTC
AGGCAGAGCACAAGGAGAGAGGGTGTCCAGGTCAGTTTCAGGACC
TGGCTGAGAGGAGGGGGCTCCTCACGGGCACCGCCTCTGGCAAGC
ACAGGGACAAGGGCAAGGACGGCATGGCCAGAGGTCCCTGGGAGC
CTCTTCCCCTCTCTTCTTCCTAGCAGCTCCCCCTCACTCTTCCCA
GGGACCCTGTCACTTTCCTTTAGCGTGTGGCAGCTCCTTGGCGTC
CCTCCCGTGCCTTCAGGTTGCTTCTGCGCCGGGCCTGCCGCTGGG
CGCC

In some embodiments, the intronic domain that promotes trans-splicing primarily in the brain comprises or consists of sequences from the gene ANAPC11. In some embodiments, the sequences from gene ANAPC11 comprise or consist of:

(SEQ ID NO: 411)
GGCCTGCGCCCCACCGCCGCGGCCGCCCTGGGAGCCGGCCCGGCC
GCGGCCCCGGCCCCCGCCCCGGCCGCCTCGCTGGCTCCGGTTCAG
TTTAATGATGTCGTCCAGAGACATGTCCATTTTGTCGGCCATGGC
GGGCGCGGAATCGGGCATCGGCTCGAGCCCGCGCGTCAGCACGCC
GGCACGTCACTTCCGGCGCCGCGTGAGGCCTTCCGGTGCCACGGC
CTCGGAGCGGCTGCTGATTGGCCGCTGGCGGACGGCTGCGCGCGC
GGGACGGGGTGAGGCGGGGAGGCGCGTGCGCACTGGCGTGCGAGA
CTCGGCGGGCGCTGTTGAGGGAGTCGGGCCGCGACTGTGGTCGTT
TTTATACCTTCCCGCGCGGACGCCGGCGCTGCCAACGGAAGGGCG
GGTAGGGCGGTGCGTGATTAGGTTGGCGAAGGTGCGAGGGCGCAG
GGGGTGGGGCGGCGGCCGTTGGAGGCGGAGCCTACGCGCCCCTCC
CTCC

In some embodiments, the intronic domain that promotes trans-splicing primarily in the brain comprises or consists of sequences from the gene ANAPC11. In some embodiments, the sequences from gene ANAPC11 comprise or consist of:

(SEQ ID NO: 412)
TTGGTTTTTTGGTCTCCACCTGCAGCCCCCCCAAGTCTGCACTGT
CACATTTTGCCAGTCATATCCTTTACAAGTTTTCTTGCGGGCCGG
GCGCGGGCCTCACGCCTGTAATCCCAGCACTTTGGGAGGCCGAGG
CAAGAGGTTCGCTTCAGCCCAGGAGTTTGAGACCAGCCGGGGCAA
CATCGTGAGACCCCCATCTCTAAAAAAAAAAATGAGCTGGGCATG
GTGGTGCACTTCTGTACTACCAGCTACTTGGGAGGCCGAGGTGGG
AGGATTGCTTGAGCCCAGGAGGTCGAGGCTGCAGTGAGCCGTGGT
GGCACCACTGCACTCCAGCCTGGGCGACAGAGCCAGACCCTGTCT
CCAAAAAAAAATTATTTTTGCAACTTATTAATGAAAACCAGTGTG
CCATTTCATTGATTTTTTTTTTTTTTTTTTGAGGCAGTCTCATTC
TGTCGCCCAGGCTGGAGTGCAGTGGGGCGATCTCGGCTCACTGCA
ACCT

In some embodiments, the intronic domain that promotes trans-splicing primarily in the brain comprises or consists of sequences from the gene NARF. In some embodiments, the sequences from gene NARF comprise or consist of:

(SEQ ID NO: 413)
TGTCATATAAATGTAGCTGGGTACAATGGCTCAGACCTCTAATTC
CAGCCACTCAGGAGGCTATGGCTGGAGGATCACTTGAGCCCAGGA
GTTTGAGACCTTGCTGGGCAACACAGTGAGACCTCGTCTCTTTTT
TTTTATTTTTTGAGACGGAGTCTCACTCTGTCACCCAGGCTCTGC
AGTGGCTCCATCTTGGCTCACTACAACCTCCGCGCCTCCTAGGAT
CAAGCGATTCTCCTCCCTCAGCCTCCCGAGTAGCTGGGATTACAG
GTGCGCCCCACCACACCAGGCTAATTTTTGTGTTTTTAGTAGAGA
GGGGGTTTCGCCATGTTGGCTAGACTGGCGACCTTGTTTCTTAAA
AAAAAAAGGGCTGGGTGCAGTGGCTGACACGTGTAATCCCAGCAC
TTCAGGAGGCTGAGGCAGGCTGGTCACCTGAGGTCAGGAGTTCGA
GACCAGTCTGGCCAACATGGTGAAACCCCCGTCTCTACTAAAAAT
ACAA

In some embodiments, the intronic domain that promotes trans-splicing primarily in the brain comprises or consists of sequences from the gene NARF. In some embodiments, the sequences from gene NARF comprise or consist of:

(SEQ ID NO: 414)
TGAGACTCCATCTCAAAAAAAAAAAAATGCAACCAGTGTATATTT
CATCATCCTATCTCTGTGAGCTTGTTATACACAAAAGTTTTCTGT
TTGTTTCATGCAAAGTAGGCCAAAAAAGTTTACAGATTTTCCACA
TGGGAAAATCATGGAGAGATTGTTTACATAAATCCCACAGTTCAA
GGGCAGAAAGAGGAATGGAAAGAGGAGTTAGAATTGAATTCTGCT
TACGTTTTATTAGTGTTTCATTTTATTGGCCTCACTTTGCCCTTT
AGATGGACTCCTGCTGTGTGTGATGGAGATTTCATAAGAGGGAGG
TCACCCAGGGTCACTAATGTCAAATCTCCTGCAGGGGAAGAAAAG
TTCCTGTGGCCACCTGTGTCTTTTCAGTGTCTTACTTCGTTTGTC
TGCAGGTGAAATTGCTCAAATAATGGAGCAAGGTGACCTCTCAGT
GAGAGATGCTGCCGTCGACACTCTGTAAGTGGCTTCTCTGGGGAG
AGTC

In some embodiments, the intronic domain that promotes trans-splicing primarily in the brain comprises or consists of sequences from the gene EPB41L3. In some embodiments, the sequences from gene EPB41L3 comprise or consist of:

(SEQ ID NO: 415)
GTGGCCATTTGTTTTCGTCCTTGGTGTTTCTGAATGTGCCCATGT
GCCTTTCCTTACCCTTTTTTTGGGCGTGAGTGTGTGGTTGCATCC
AGCTGGCTTTGCCTTCAACATCTCAAAGGTGTCTTCACTTATTGT
CCTTTGTGTCTGCTTGGCATATCTGTCACATGCCTTTAGTTGCAT
CTCAAGAATGTACTTCACTATTCGAAAGGCGATCATATTGAGAAA
TTTTAATAGAATTAAATATCTAAAAGCATTATGTCTCACCAGTTG
AGTCTAAGAAAGCAGCCAAAACCAACGTGAATGCGCTGCAATGCT
TTGGTTTTTGTTTTGTTAATTGCACTTTATCCTGCCTTCCGTTTG
TTTTGCGGATGTCCTGAAGCAGCAGCGCAGGTTCAAGTGCATGCA
CCAGGGGCAGGGGCAGGCATCAGAGATTTCAGTGGGAGAAATGAA
AGCTAATTTATATTTTTCGTCAGTGTTTCTGGTTATAAGCACTTG
CTGC

In some embodiments, the intronic domain that promotes trans-splicing primarily in the brain comprises or consists of sequences from the gene EPB41L3. In some embodiments, the sequences from gene EPB41L3 comprise or consist of:

(SEQ ID NO: 416)
GTGTCACTCATTGGGGCCACAGTTTGCAATTCCAGCATGACAGTT
TTTGAGGCAGTCCCCTTCCACCCCAAACCTTTCCGTTTCTCTTGC
TGCTTGGCTTTATGTTGAGAGCAGCCAACCCAGCTTCTTAATTTT
TTTTCATGGTTAAATCAATACCGCAGTGCAAAGTCAGCATTAAAA
CACTTTCCATTTTATTTCCAGCTCCCTGTTCCCAAGATGTGATAG
TGCTGCTGTGGCTTGCAATGAAGGGGAAAAAATATATATATCTGC
TTAAATCGGCGCCACCCTGATCCTAATAACCGTCTCTGCTTTACC
AAACAAGAGCACCTGGTTTTCTATTTTATTTCCCATTCTTTTCGT
TTCCAAACTCTGAGATTCCTTCGTTCTTCCTCCTCCCTGTCTCCT
CTGCCTACGATTTTGTCTAACAAGCTGTGAATGTTTATGTGCCTG
AGTTTGTGTCTCTCTTTTGTGCTTGACTCTGCCTCTCTTTGTTCA
CTCA

In some embodiments, the intronic domain that promotes trans-splicing primarily in the brain comprises or consists of sequences from the gene PSMG2. In some embodiments, the sequences from gene PSMG2 comprise or consist of:

(SEQ ID NO: 417)
GAGATGGCCGCACGGTCTCTAGCCCGCAGGGCTCTCAGAGTCGCA
GGCGGTGACGCCCGGTCGGGGGGCCCGGTCGCAAGGGACACACAG
CTGGGGGCGCAATGGTGAGGGCGGCGACGCACGGTCCGGAGGGGA
TGCGCGGCCGGGTAGGGCGTGCAGGGTTGTGGGGCGTGGGGGACA
CGCGGGGCCGGGCGCGCGGTCGGGGGGCGCAGGGGCAAGAGGGTG
TCGCATTTATCAGGGAAGTCCCCGGCCGACTCTGGCGGGGAACTG
GGCGCGCCAACCCGGGTCACCGCCCTGCACAGCGGGAGAAGTCAC
CCGCATTGACGTTCCCTAGGCGCTGGATCGCCGAAGGTCTGGTTG
AAAACTTGAACCCGCCTGGAGCGGATGCCTAAGAAAATGCAGGCC
CGCTGAAGCCTCCCCGCCGGGCCCTGGGCCCTGAAAGCCTCCGGC
AGTTTCCCGTGACCCCGCGGCCGCTCTGCGCGTTTTCCGATTTCT
TGCT

In some embodiments, the intronic domain that promotes trans-splicing primarily in the brain comprises or consists of sequences from the gene PSMG2. In some embodiments, the sequences from gene PSMG2 comprise or consist of:

(SEQ ID NO: 418)
TAAGCACCGTAAAGACGGGGATCTTTACGGGTTTTCCTCACTGAC
TTCTCCCAAAAGCTAAACCAGCGCCTGGAATATAAACATGTACAT
AATTTGCAGGTGTTAGTTTTAGCGTCCTTCCGGATTCATTTGTAA
CATTGGTCCCTTCCCTCACTAAGGTAATGTCTCAAGTACCACACT
CATCAGAGCATGTGGAATGAATATCACAGTGGTAAAATTGAACTG
AGGGCAATTAGCCGTAGAAAGAAAGAATTCCGCCCCCCCAACCTA
TTAAATATGCAATAAAACAGATAATTCTTAATCACCCATTGGTAG
ATTAATTTAATCGATAAAATTCCACTGAGGCTAGTCCTGTGTGAA
AGCCCTACAGAGTGTTGAAAGCATCAACCAAATAGGCAATGTATA
TTAAGTTCCAGTAAACCTTTTACGGCAGTTTTATGTAAAATGTTA
GAAATCACGCTCTCAACAATTGCATCTTGCGGTAGAAACTACAAC
AGTC

In some embodiments, the intronic domain that promotes trans-splicing primarily in the brain comprises or consists of sequences from the gene DTNA. In some embodiments, the sequences from gene DTNA comprise or consist of:

(SEQ ID NO: 419)
TAATTCAAATTTCCTACCAAAGAAAGTTAACCGTCTCTTGAGGCA
TTTGAATTATTTAGAACTGTTTTCAGATAATTTGCACATCTTTTT
TAGGGCCCTATACACCTGCCTCAGGAAAGAAAAAAAAATCTGTTT
GTCAGATGTAGGCAACACCACTCGTTAAGTAAGCCATTAGTCACT
CTATTTTTTAAAAAATAACTTAATTATCTCATCCACACCAGTTTT
CCATTCTCTGCCCATCTTCCTTTCTTTTTTCTTCCTCTCATTCTC
CGTGTCTCTGATATTCGCTCTCTTCCCACTGGACAATCAGCATTC
ACCCCTTGTGCTTCTGAGTCTTCCAGTGAGCTGTGCTGGTCTCCT
GGAGACAGAGAGCAGTTTGGGGTAAAGGCAAGTCTGTTTGGCTAA
TTTAAATTCTTGTAATGTGAGAGATGGGAATAGAAAGAGCAAATG
GCATTACAGAGATTTTTGTTGTTAAAAGGACACCTCTTCTCCATC
CCAT

In some embodiments, the intronic domain that promotes trans-splicing primarily in the brain comprises or consists of sequences from the gene DTNA. In some embodiments, the sequences from gene DTNA comprise or consist of:

(SEQ ID NO: 420)
CTTCCATCCGGTTGAGTGTTCCTACTGCCACAGTGAGAGTATGAT
GGGATTTCGCTACCGATGCCAACAGTGTCACAATTACCAGCTCTG
TCAGGACTGCTTCTGGAGGGGACATGCCGGTGGTTCTCATAGCAA
CCAGCACCAAATGAAAGAGTACACGTCATGGGTAAGGCAAGGTCC
AGGCAATACTTGGAGTTGGATGTGTGAGTGTTGCCCAGAGTTAAA
GGGAATATCAAGGATGGTGGCAGAATTAAAGGGCAAACTTACCTT
CCATCCCAGGTCTAGTATTCAGTCCCCCTTCCTCCCACTCTCCAC
CCTACTGCGTGCTCTTACTTATTCTGTTGGAACCCAGTGTAGCTT
CCTGAGATTTAAAATATAGGAAGACTCAAAACTATGTTACTTCTT
GGAATATAGATAATTCAGTACTGATTTAAATAACAACTGAATAGC
AAGGACCTTCTGGAACATAATCTTACATTCACAGTAAGTTAAAAT
CTTT

In some embodiments, the intronic domain that promotes trans-splicing primarily in the brain comprises or consists of sequences from the gene MAPRE2. In some embodiments, the sequences from gene MAPRE2 comprise or consist of:

(SEQ ID NO: 421)
AGCCTGCACTCCATTTTCCAAATAGAACCCAAATATCAACAGCAT
CAACAAGCAGATTTGAAACCCCCCAAAAACACGCTATTATTAAAT
TGTTAATTGTATTAGAATTGCCTAGTTTAATTAGTAATATTCATG
ACAAAACTAGTTTGCCCCTTATCATCAATCGTGCTCCAGGTAGGA
GTACAAACACTGGGTGTAAAAAGCAGATGCACCCCAGTTGGTGTG
CAAGCCCCCGTTTGCAGGTAATTAATTCACAGTGTGGCGGCGCCT
TTTCAAGCCCCCTCTGCAAGCCGCGCCGGCCTCCTCCCTCTCGAG
CGGGGTCAAACCACTTAGTTTATTTCACCCGCTGGCACTTTCTTC
CCTGTCACGAATTCAGCCTGGCTTCCTTTGGCCTGAGACAGCGTC
TTCAGGCGGGTCGGCTCGGACCTGCCCAGTGGGGCGCCCACCTGC
ACCCGCACCCGCACCCGCACCCGCGGGAAGGGGCGGCGCTGGCCG
GACT

In some embodiments, the intronic domain that promotes trans-splicing primarily in the brain comprises or consists of sequences from the gene MAPRE2. In some embodiments, the sequences from gene MAPRE2 comprise or consist of:

(SEQ ID NO: 422)
TGAGTCAAGTGCATTTAAAGCGGTACCGCCCTGGCGCACCCCGAC
ACCCCCTGCACTGAAGCAGTCCCTCCCAGGCCAGGGGCTGGCTGC
GCCTCCTCCTGCCGCTGAGGTCTGCAGCCCGCGGGGCTCGGAGCA
ACCGGAACCGGGGGTCCAGAGTCTAGCAAGAAGTTGTGCAAGGGT
CGGCTAGGCGGTCTCCGGGTGTGCACCCCGCCGCTCTCCCGCGGG
CCACATCTCTGGTTCGGTGCCCCAGCTCTGTGCACCCCAGGAAAG
TGGAGGGAGGGAGTTGGGGCTGCCACGCTGGCAGAAGCGAAGTGG
ACTGGTTCTGAGGGGTGAGGCTGGGGCTGTCCAGGGTAGGAACAC
GGCGTTGGTGAAGGAGGGGGGCTCGCGACTGCGCGACCGCGGCGC
ACGACCCTCCAGGCTCTAGCTGGGGCGCCGGCACTGAGAGAGCTG
GGGGCTCTGGCAGGGGCTGCGCGCTTGGGGGCAGATCAGGAAGTG
CAGC

In some embodiments, the intronic domain that promotes trans-splicing primarily in the brain comprises or consists of sequences from the gene CNDP1. In some embodiments, the sequences from gene CNDP1 comprise or consist of:

(SEQ ID NO: 423)
CCACTCCTTGTGGAACCTCAAGAGGGTGTCTCAGCTTCCCAACCC
CATTAGGGATTGGTCCTTCCTTCTGAAGGCTCCCATGTCACTTAA
AACTAGGAGCAAATCAATCTGTTATGCCTCTTCTCCAATTCAACT
GCCCTTTGCAAGCTGATTTTGCAGCGAACCTTCAGAGAGCTGGGA
AAACTCTACCCTCGGCCCCTGCACAGCCCTAGGAAGCTAATCCTC
GGCCGTAACATTCCATCACAAGGGCCCACAGCCACAGTGTGTGGG
GGCCTGTCATGTGAGGGGGCTGTCACCTGAGGGGGCTGTGCCGCT
GGCTCAGCTCTGCCTGCTGGCCGGGGCCCTACTCCCTCTCAGAGA
TGACACACAAGTGCCCCCAAAGAAGAACCCGAGGAGTCGCAGGCT
GGGCCAGGCTGCTGGCTTCCAGTGACATCACATGACTTTGGGGCC
ACTGCCTCATCCCGTATCTGCGCGTATGAGATGCATTGTCTCTTC
CTCT

In some embodiments, the intronic domain that promotes trans-splicing primarily in the brain comprises or consists of sequences from the gene CNDP1. In some embodiments, the sequences from gene CNDP1 comprise or consist of:

(SEQ ID NO: 424)
TGAGTAGGACCTCCTCCATCAGAGTCCGTGCATTGGGTGCCAGAA
TTCCATTTGTCCCGGGAGCATGTGCGTTAAGCCCAGTTGGGCAGG
GCAGGCACCCAGCGTGGCCCCAGATGCTGCTCCTCATGGTGTTCT
TAGGTGCTGTCTTGGCCGAGGAATGTGGTATTTGGAATTATATTT
TATCAGTAATGCTTTTGGGTAGTTGTGTGTTCTTAACAATAATTA
AATGGCATTATTGTCAAGTAGTTCATATTCAAATTGTTCATGAAC
TCAGTCTTCTTAAATCAATTTTTTTCTGTACTTTTTCTCTTTAAA
AAAAATGAGTTTGCTTCCTGGAAGAATTTGCCTAAATAATGGTAA
CAGACAGTGTGCTGTTTGTCCTGGTCCCAGGTTCCCGACTTAAGG
GCTAGTTTGCAAGAGCTAATCACCGGGCTAAATCACCCCAGGCCT
TTCTCGGCGAGGTTTCCATGGTTCAGGTGCAAACCTGAGTGTGGG
TGTG

In some embodiments, the intronic domain that promotes trans-splicing primarily in the brain comprises or consists of sequences from the gene C19orf25. In some embodiments, the sequences from gene C19orf25 comprise or consist of:

(SEQ ID NO: 425)
GTGGGAAGAGGCCGGCGACTGGGGTGACCGCGGGGAAACTGAGGC
AGAGGTTGAGCCAGACCCTGAAGCCTTGGCCACCGTGTTTTTGGG
CGGAGGGAGGGTCAGGGCCTGGGGCGAGCTCAGAGGGGCGGGACA
GGGCGGGCTTCCCCGGGAGCGGGACCGCGACGGGTGCGTACGAGT
TCGCTGGGCGGGAGGAGCAGCCCGGGCAAGGGCGGAGAGGTGGAC
GCCCCGGTCAGCGCCCCCTTCCCCGCCTTCAGAGATGGGCTCCAA
GGCAAAGAAGCGCGTGCTGCTGCCCACCCGCCCAGCGCCCCCCAC
GGTGGAGCAGATCCTGGAGGATGTGCGGGGTGCGCCGGCAGAGGA
TCCAGTGTTCACCATCCTGGCCCCGGAAGGTAGGGGGAGCCCGGT
CCCGGGGAAAAGCGGACCTGAGACCCCGGCAGCAAGGGGACGGGA
GAGAAGCACACCCCTAACTCCTGACCCCACGCCCTGGGAACCCCC
GCAA

In some embodiments, the intronic domain that promotes trans-splicing primarily in the brain comprises or consists of sequences from the gene C19orf25. In some embodiments, the sequences from gene C19orf25 comprise or consist of:

(SEQ ID NO: 426)
ATGGTATGTAAACTGCATCTCAATAAAGCCCCTGAGAGAGGAAAA
GGGCCCTCTGCTCTTTCTGCCTTCCCAGGCATCAGCGGAGAGGAA
AAGGTGACCTCCAGCACCGTCCCCGAGGTGGGAGGACCCGCCTTG
GCCGGAAGTGGAGGCGCCGCGACCGCCCCGCCCCCGCTGTGTTCC
GGCCGCCGAGTGCTCATGCACCACCAGGCTCCCTTAGGTGGACAG
CAATTTTGTGCTCCCGTCAGCCACATGGGTGGCGTGTCCCCTAGT
GCCGTGTTCCCCTTTTCAGAAAGACGAGCCTCGGATGCTTTTCTA
CCCAGCCAGTGGAGCCTGGACTCGAACTCGCTCTCATCGTAGCGC
GTCCAGGTTCGGGTTCGCCTGAGAGGCTGAGATCCGAGTCTCAAG
GGCACTATCCCTTTAAGACGGGGCGGAGCCCCGCGATGAGCGGCG
GGCCGGCGGGAGAGGAGGCCGGAAGTGTGTGTCACGTGTTCGGGG
ACGG

In some embodiments, the intronic domain that promotes trans-splicing primarily in the brain comprises or consists of sequences from the gene SHD. In some embodiments, the sequences from gene SHD comprise or consist of:

(SEQ ID NO: 427)
TCCCTCCTGGAATCCTTCCTAGAGCTGTCTGGGATTTCCTGTTAG
AAACCACATCTTTCAAATAAGGAAAACCAAGACTCAGGGAGTGAC
AGACCCTGCCCTCAGCCCCGCCGGGCTGCCAGGAGGCCCCAGTCA
TAGCAGTGTTGGTTCTGACTTCTGTCTTGTCCTCCAACTGTGCTC
TCCAAGCTATGACCTGTGCCTGACCAGGGGGCTGGCCACAAGAGG
AGAGAATCTGGGGTCCCATCCTTCCTGACCACCCCTCTCTCACTC
CCCTCATTAGGAGGAACTGGGCAAGCCCCCTTATGGGGAAAGAAA
AAAAACGGTTCTGCACTGGAAAATTGGCCAGTGGCTCCCTTTTCC
TCTCCCACCCCCCAAACCTCTCAGTGCCCTCACCCTCAGACAACG
ACAGCTGACAACAGGCAGCCAGTTCAGGCCAGACCCCTCTCACTT
CTGCCCCTCTTCCCGCCACCGCTCCCCCCCACCCCGCCCCCAGAA
GCCA

In some embodiments, the intronic domain that promotes trans-splicing primarily in the brain comprises or consists of sequences from the gene SHD. In some embodiments, the sequences from gene SHD comprise or consist of:

(SEQ ID NO: 428)
TGCGTGGCTGGGTGGCCTGGGGAGACTGGGTGGAGGGGAGGCTCA
GGATGGGCTCTCTGGATCGTGGAGAGCTTATTTTATGTGTGATCA
CAGGAGGGGACTGGGGCACCAGACAGACCCTTCCCAGTCCTCCAG
ACCTCCACCTGCTTGGGGAAGTCTGTTCTTTTTATGTATTTATTT
ATTTTGAGACAGTCTCCCCCTGTCACCCAGGCTGGAGTGCAGTGG
TGGGATCTCGGCTCATTGCAACCTCCACCTCCTGGGGTTCAAGCG
ATTCTCCTGCCTCAGCCTCTCCAGTAGCTGAGATTACAGGTGTGT
GCCACCACACCCGGCTAATTTTTGTATTTTCAGTAGAGACTGGTT
TCACCATGTTAGCCAGGCTGGACTCAAACTCCTGACCTCAGGTGA
TCCTCCCGCCTCTGCCTCCCGAAGTGCTGGGATTACAGGTGTGAG
TCACTGCGCCCGGTGGATATCTGTCCCTTTTCAATGCATAATGCT
GGTT

In some embodiments, the intronic domain that promotes trans-splicing primarily in the brain comprises or consists of sequences from the gene UBXN6. In some embodiments, the sequences from gene UBXN6 comprise or consist of:

(SEQ ID NO: 429)
GTAAGTTCATGGGAGGGGCCTGAGAGGAGACGGGTGAGACAGACG
CGTCCGAGCAGGTTGGGAGAAAGGGCTTCTCTGCGAGGCTTCTCT
GCCAGGTCACTGTGCCACGTGGGTTACGGAACTCTCCTTGGTGTA
GGGCTCAACAGGGTGGAGCCACGAGTCCATGAGCTCTTCGTACTT
GCACAGCCCTAGGCCTGGCATGGAGTGGCCAGGGCGAGGCTGGTT
CTGCTGTCCGCTCTCCCCAGGGTCCCGCCACGAGGGTGGCGTGGC
ATGGGGCACATCGCCCGGACTTCCTGTCCCTCTTGGGTTTAGGAA
TGGGATGGTGAGGCCTCCCTGGCAGCTTGTTAGGAGGAGTAAACC
AAGCGAGCCGACCAGCTCCCAGCAGGGTCCTGGCACACAGTAGGT
GCTCAGCAAACGAAGCTGTGACCTGAGGGTGGAGAGTCTTATGGC
AGGGAGGGAGGGAGGATGTGACACCCCTTGAGTCTCCTCAATTCC
TGAG

In some embodiments, the intronic domain that promotes trans-splicing primarily in the brain comprises or consists of sequences from the gene UBXN6. In some embodiments, the sequences from gene UBXN6 comprise or consist of:

(SEQ ID NO: 430)
TTCTCTCCCTGCGGCAGCTCTAGCCCTTTCCAGACCCCCATCCTC
CTCCCACTCCCCCGGAGAACATTCTGGGCAGAAGGTGCAGACAGT
GCAGGGGCCTGCAGTGGGGAGCAGCTGGGGTGTGGGAAGAACTGC
GGGGAGCCCAGGGGAGACAGGCCACGGGAGGGCAGTGGGCCAGGC
TGGGAAGGGCTCTCTGGCCAAGGGAAGCTTTAGGATTTTGCAGTG
GGAGGGCGTTGTGCAGTGGAGTGATGCTGACGCTCCCTGGCTGAG
GTTCCAGAAGGGTCCCTGTGGCTGCCCAGGGGGGCAGGGTCCATG
GCTGGTTGGGGTGAGGTGCCCTGGGAAATGGCATTTTGTTGGAAA
GACCCAGATATCGGATGAGGCCGGGGTGGGTCTTCATGAGTCATC
TCCTCTTCCACCCAAGATGCTCAGCTGTGGGAGGACTAGATAGGC
AGGGGTGGGACGAGGAGGGAGGGCTGCCCGGGTATGTAAGCAGAG
CCAG

In some embodiments, the intronic domain that promotes trans-splicing primarily in the brain comprises or consists of sequences from the gene TNPO2. In some embodiments, the sequences from gene TNPO2 comprise or consist of:

(SEQ ID NO: 431)
GTGTGTGTAGGGGCCTGTTTCGGTGGGGCTTGGTTGGGTCTGATT
GGGGGAGGCCGCACTGAAAAGTTGAGCGGGGTGGGGGGAGGGGAG
CTCGTTGGGTCCTGACAGGGAGGGGCGTCAGACTTCGGCCTAGAG
GGTTTTCTTGGATCTGACTTCGAGGCGTTGCTGGGATCCGTTGAG
GGGAATGCCCCGGGGGGTGTTTACCCAGGCCGAACTGGGGGAGGC
TGCAAGGATCGGACCAGGGGGTTACTGACACGAGGTGGTGGGGGG
CTCGGCCGAACTTGACTGGGAGAGGCAACAGAGATCCGTCAGCAG
GGGTTACTGCTGACAGGGTCGGAGGGAATTTCAGGGAGAGGCTTG
GCCAGACCTGATTGGGGAAGGCGACTGGGATCGGTCCAGGGGGCT
CCTATAGTGGGTTGGCCGGGCTTGGCTGGCTCTCATTCGAAGAGG
CAGCAGAGACCTACCTGGGGACTGCTTTGGGCCTGATTAAGGGGA
ATCC

In some embodiments, the intronic domain that promotes trans-splicing primarily in the brain comprises or consists of sequences from the gene TNPO2. In some embodiments, the sequences from gene TNPO2 comprise or consist of:

(SEQ ID NO: 432)
GAGGACCGGCTGGCTAGAGAGGCCAAAGAAACGGCAACTCTTTGG
TCACGTGACCGGACCTTGTCTCAGCCCGGGTTTCCCAAGTGCCAG
GGCACGTGTGAAGGTTGAACCTTCCGATTGGCTCGGCGGGACCAG
TTTGAACGGAGTACGGGAGCCGCGGCGGCTCACCTACGGCCGCGT
TTTTCTTTTTTTTTTTTCTTTCTTTCTTTTTTTTTTTTTTTTTTT
TTTTTAATTCTCTCTCTTTGGCTCCCTCCTTCCGCGCGAGTCTCT
GGAGAAGCCGCAGCGCGAGTTGCCGCCGCTGCTGCCCGGGGCCGG
GTAAGTGGGCCTCACTCAGAGCCCGACCCTCTTGGCCCCGGCTTG
CGTCGACCCCCGCCGGGCACCGAGCCTGCGCCGCGCGCGGCCCGG
GCGTCGGGGCCGCGCCCGACCGGGAAAGGCCGGGAAGCCGGTTGG
GCCCGATCCTCCTGGCAGCTAGAACGGGCCGGGCGGGGGAGGGGG
GAAC

In some embodiments, the intronic domain that promotes trans-splicing primarily in the brain comprises or consists of sequences from the gene NFIX. In some embodiments, the sequences from gene NFIX comprise or consist of:

(SEQ ID NO: 433)
TTATTATTTAAACTTCAAACAAGCAAACAACCAAAAAAAAAAAAA
AAAACCAAACAAAACCGAGAGAGCCCATCCTTCTGTCACCTGACT
GAGTGGGAAAAAGGGTGAAGGGGTTGTGGGAGGCTGGGGAAGGGG
TCGCAAGAAGACCCATGTAGCTTTTAACCCTAATGTGGCCGAGAC
AAGCACCTTATTTGTGCTAACAAGAAGTGTTTTGTTCATTATTAC
TGTGATTAACATTAGTATTGGTGTCGATAACAAAGCTGAAATCAC
ATATTTAGGATTTAGGTCTGATTAAAAAATGTTGGGGTGGATGTT
CCAACTGGATCAGGAGAAAAGAAAATGAAAACAGCCTGGGGAGAG
GGAAGCCTGATCTGTTTCCTCACTCGCTTGCTCGTGGATGTCATT
TTCTGTCTTCTTGGGGGCGGCCAAAAATCGACCGGTGTCGGGGAC
CAGAGGCGGCCCCGCACGCCCCCGCGTGTGCGTCCACGGGCGTCT
GTGC

In some embodiments, the intronic domain that promotes trans-splicing primarily in the brain comprises or consists of sequences from the gene NFIX. In some embodiments, the sequences from gene NFIX comprise or consist of:

(SEQ ID NO: 434)
TGTGTGGACGGCAACGTTGTCTGTGCGCGTGTGTGTGAGTGAGTG
AGGGAGAGAGAGAGAGAATAGGTGTGTGTAGAGGCTCCCGGTGCC
TCTGTCTGGCTGCTGAGGCTGAGATGGGAGCAAGTGGCTGGCGAA
GCTGGTGGTGGCTTCAAACCACACTTTCGTAGAACAATCGCAAGA
GAAAATTGTTGGGGGGAGGGAGGAGGAGGAGAAGGCGGTTTTCCT
TGTGCCCCCCCTTCTAACGCTGCTTTTCTCCTTCTCTCTTTCCCC
CTCATCCCGTCTTCCCCTCCTCCCGTCCTCCCTCGCCCCGCATGC
TCCCGGCTTGCCGCCTGCAGGATGAGTTCCACCCGTTCATCGAGG
CACTGCTGCCTCACGTCCGCGCTTTCTCCTACACCTGGTTCAACC
TGCAGGCGCGGAAGCGCAAGTACTTCAAGAAGCATGAAAAGCGGA
TGTCGAAGGACGAGGAGCGGGCGGTGAAGGACGAGCTGCTGGGCG
AGAA

In some embodiments, the intronic domain that promotes trans-splicing primarily in the brain comprises or consists of sequences from the gene RHPN2. In some embodiments, the sequences from gene RHPN2 comprise or consist of:

(SEQ ID NO: 435)
GTAAGTGGGAGCAGCACACCTTCCAAAAGCCTCTGAGCCAGAGAT
CCTTCGTACATCCAGGGTGCTGCACAAAGAGGTCAGATAGCGTTC
TAGACTGGGGTGTGGCAGCCCCCACTTTCGGAAGTGAGAGAACCA
TCAGGTTTGGGGTTGAGTGAGGTGCTAGACTGGAAGGGATGAGCC
CATTTGTTGGGAAATACCTGCAGTGTTGGACAAAAAGGCATTTGT
GAGGCCGGGCACGGTGGCTCACTCCCGTAATCCCAACACTTTGGG
AGGCTGAGGCGGGTGGATCACCTGAGGTCAGTCTGGCCAACATGG
TGAAACCCCGTCTCTGCTAAAAATAAAAAAAAAAATAGCCAGGCG
TGGTGGTGCACACTTGTAATCCTGGCTTCTCAGAGGCTGAGGCAG
AATTGCTTGAACCCGGGAGTTGGACGTTTCAGTGAGCCGAGATCA
CACTACTGTACTCCAGCCCGGCTGACAGAGCAAGACTCTGTCTCA
AAAC

In some embodiments, the intronic domain that promotes trans-splicing primarily in the brain comprises or consists of sequences from the gene RHPN2. In some embodiments, the sequences from gene RHPN2 comprise or consist of:

(SEQ ID NO: 436)
ATGCACCACCACACCCAGCTAGTTTTTGTATTTTTTAGTAGAGAC
AGAGTTTCACTATATGTTGGCCAGGCTGGTCTCGAACTCCTGACC
TCAGCTGATCCACCTGCCTCGGCCTCCCAGAGTGCTGGGATTACA
GACGTAAGCCACCATGCCCGGCTGGAATCATTCATTTCTTTTCAA
GTGGGTATCTTATGGTATTTTAGGGCATGGCTGGGAGCAGTTTTG
TTTTCTCTTCTCAAGACTGAGTGTTTGCAGGATGTCATAGAGTTC
ATGTCTGCAGCTCACAGTGTCATTGCCTGTGTCCCCAGCTCCACG
TACTGGCAGGTGTGCTGCAAGCTGGGTAGGTGCCCTGTGTCCCTG
GGATACCTTAACCGACACTCCTGGCCCTCCTCTGCAAGCTGTGCC
CTGATCCTCCCTGCAGGGACTGGGGATTGGGTCTGCTCACCTAGA
AGCCAGGATACCTGGCTGAGGGCACTTCTCTCCCTCTTCTCTTTG
AACA

In some embodiments, the intronic domain that promotes trans-splicing primarily in the brain comprises or consists of sequences from the gene SCN1B. In some embodiments, the sequences from gene SCN1B comprise or consist of:

(SEQ ID NO: 437)
AGATGGGGCTTGGGAGGATGACTGAGGAACGTGTGTGTGCTCGTG
TGCATCTTGGCTAGAGGCAAAAGCGGGGTCTGGTTGACTCCAGGC
GTGATCCTAGCCCCCCACCCCTGTGCCCTCCCTGCCTGAGGTCAA
GGTGTCTGAGCCCATCTGTGTGCCATCTGTGTTTGTGGGTGTCAG
GGCAGGGGACAGAATCAGGGTCAGGTAAGGGAAGAGAGGCCCAGG
CAGTGACACCTTCCCCTCCCTGGCTACCCCTAGATCCTGCGCTAT
GAGAATGAGGTGTTGCAGCTGGAGGAGGATGAGCGCTTCGAGGGC
CGCGTGGTGTGGAATGGCAGCCGGGGCACCAAAGACCTGCAGGAT
CTGTCTATCTTCATCACCAATGTCACCTACAACCACTCGGGCGAC
TACGAGTGCCACGTCTACCGCCTGCTCTTCTTCGAAAACTACGAG
CACAACACCAGCGTCGTCAAGAAGATCCACATTGAGGTAGTGGAC
AAAG

In some embodiments, the intronic domain that promotes trans-splicing primarily in the brain comprises or consists of sequences from the gene SCN1B. In some embodiments, the sequences from gene SCN1B comprise or consist of:

(SEQ ID NO: 438)
CCGCACACCCCTCTGCACTCCTGCCCCGGGGGAGCCGCGCTGGCC
TGCAGTCTCACTCAACACCTCTGGGGCGGGCCTAGGGGTCCTTTC
TAAAGCTCTCAGGTGAGGCAGTGAAGGGCCACAGGTGCGGGGCCC
ACCCTCTGAGCAGCAAGGGCCTACAGCGCTTTTGAACGTCTCATC
ATTTTGCCTCAGCCACCCTGGACCCCTTTTCTGAGGGAGGTCCGC
CCCTTCTCTGATCCATCTTCTGCTCAGCATCAATTGCAATTGTCC
AGCGCCCTGTGGCCACTGGCTTAGAGTTGGGGAAACAGCCCAGGC
CTTGAAGCAGCTACAAGCCGTGGCAGAAAGGTCTCTCAGGGCCAC
GGCACACACATCCTCATGGGCAACACGCACTGCCCATCTTTTCTG
TGAGATGGGTGCCGGCCTCAGTGCCTTACACGGAGTCCCTTAGTG
AATCCTCTCCCCACCCTCTGGGGTAGGGGCTCTTCCTATCCCCAT
TTTA

In some embodiments, the intronic domain that promotes trans-splicing primarily in the brain comprises or consists of sequences from the gene SAMD4B. In some embodiments, the sequences from gene SAMD4B comprise or consist of:

(SEQ ID NO: 439)
TGTATGGAGTATGCAAGTCTCAAATATCTCCAGCCTGCACTCATT
CATTAATGCATGGAGGCACTATTCTAGGTACTGGAGACAGAGCAG
TGAATAAAACATTCAAGGTCCTGAAATTCATGGAATTTATATTCT
GATGGGGGGAGGAAACAAAAAAATCAGAGTGTGATTCATGCTGTG
AAGGAAATAAAACAGTGTGAAAGAGAGGGTGGGAGTGGCCACTGT
AGTAGACAGGGGGATCAGAAGAGGCCTCTTTTGGGAGGTGACAGT
TGAGCCTGATAACCAGAAAGATGAGAAGGCACCAGTTGTGGGAAG
ATCTGGGAGTGGAACATTCTAATCTAAGCATTGTTACATGCAGAA
AAAGGACCTCTGCAAGGTGGGGAAATTTATCACAGATATTTATTG
AGGGCCTGCTATGTACCAGGTGCTATGCTAGGAATTGGAGATGTA
GCAATGAATAATATAGCTACAGTCTTCCCTTCTCTCATGGATCTT
ACAG

In some embodiments, the intronic domain that promotes trans-splicing primarily in the brain comprises or consists of sequences from the gene SAMD4B. In some embodiments, the sequences from gene SAMD4B comprise or consist of:

(SEQ ID NO: 440)
TGAGCCCAGATCTGATTGATGAGAAGGAACCAACCTTGTGACGGT
CCGGGGAAGAGCATTTCAGGCAGAGAGGTCAGCAGGTGCAAAAGC
CCTGCCATATGTTCAGGGAATCGCAAGGACCAGTGTGGCTAAAGC
CTAAGTGAGGGAGAGGGGTGAGGGTTGGAAAGGCTGACAGAAGCC
AGATTACTTGCAGCCTAAGGGCCACAGTAAGGAGTTTCTATTTTC
ATTTAAGGGGATTGGGAAACTATTAGAAGGGAGTTTTTAAAAGCA
AGACAGCGACATGATCTGATTAATTTTTTTAGAAAGCTCACTTTG
GTTTCTGGGAGGAAAACAGAGGGTCAGGACAGAATCAGGAAGGAA
AGTTAGGAGACAATCCATTTGTCCAGATGAGAAATGGTGACATGA
ATGTGGTGGTGGTGGTGGTAGTGAAGATGTAGAGAAGCAGATATA
TTTTGGGTGTACAGAAAACAGGACTTGATGATGGGATTAGAGAAA
GAGA

In some embodiments, the intronic domain that promotes trans-splicing primarily in the brain comprises or consists of sequences from the gene PAF1. In some embodiments, the sequences from gene PAF1 comprise or consist of:

(SEQ ID NO: 441)
GTATGTAGTAGGCTATACTATGTACGTTTGTGTAAGAGTACTGTA
TGATGTTCACATGATGTATATCTCAGAATGTGTCCCTGTTAAATG
GCCCATGCCCGTATTTATGTAAATACACCGTATAACACACACACA
CAGTGTAGGGCAACCAAACTGGTGCCATGGGAGCCATCCTTCCTT
TCCCCTGCCCTCAATACCTTGAGCTTAGCTGCCCTTATTCAATCT
GTATTTATTCCCATTTACTCTAAGTATTGATGGGGAACCAAGTCA
GACCAGACCCCTCACTGGGGGGCGGGGAAGTGGGAGGTGTGTCTT
TAACTGCATCTTCCACTTCTCTCTCCCAAATTCACCCCCTGCAGG
GTCCGCCTTAGTAAGCGCCGGGCCAAGGCTGGGGTTCAGTCAGGC
ACCAACGCCCTGCTTGTGGTCAAACATCGGGACATGAATGAGAAG
GAACTGGAAGCTCAGGTAACAAGCACCACTGGCTCAGGGCACCCA
TGGC

In some embodiments, the intronic domain that promotes trans-splicing primarily in the brain comprises or consists of sequences from the gene PAF1. In some embodiments, the sequences from gene PAF1 comprise or consist of:

(SEQ ID NO: 442)
ATGAATGTTTTAAATGAAATGCTTAAACCCATGTTCAAAAATGTG
ACTCCAAAACTCTTCCCTTATGGCTCAAAGTTTACTGCTATGCCA
GCACCTCTGGTGTAGAGTTGGGAAGTCTTGAGTTTCTGTTTAAAG
CAGAGATTACAAGAACTGTGACCCCATAGGCCAGGCAGGAAATGT
AAAAGAGCAGTGGCTGGTAGCATCTGAGTGACCTGGAGTTCTGGG
CAACCATGGAGGGCACTATTCACTTCCAGCCCACTGTTGCTGTGC
TGTGGGGCCAGAAATTCCTATCTTCCAAAAGCCCCAAATCCAAAT
GATTTGACATCTCCTGGTTTCTCAAAGTGTTGGTAACTAACTGAC
ACATACAGTAATGCACTGCATGTTTCAGTCAACAATGGACTGCTT
ATGGTGGTCCCTTAAGATAGAATACCGTATCTTTACTATACCTTT
TCTATGTTTAGATACACAAATACTTACTATTGTATTACAATTGCC
TGCA

In some embodiments, the intronic domain that promotes trans-splicing primarily in the brain comprises or consists of sequences from the gene CLPTM1. In some embodiments, the sequences from gene CLPTM1 comprise or consist of:

(SEQ ID NO: 443)
GGCTCAAACATAGATGTCACTTGTCACCAGCCTGGCCAACACGGT
GAAACTCCGTCTCTACTTACAAAAAAAAAAAAAAAATGGGAGGCT
GACGCAGGAGAATTGCTTGAATCCGGGAGGCAGAGGTTGCAGTGA
GCCGAGATCGCGCCACTGCACTCCAGCCTGGGTGACAGAGCGAGA
CTCCGTCTCAAAAAAAAAAAAAAAATTAGCCAGGAGTGGTGGCGT
GCTCCTGTAATCCCAGCTACTCGGGAGGCCGAGGATGCAGTGAGC
CGAGGTTGCAGTGAGCCGAGATCGCGCCATTGCACTCCAGCCTGG
GGGAGAGTGAGGCTCCGTCTAAAAAAGAAAAAAAAAGAGAGAGAG
AGACGTCACTTGTCCAGGATATCTTATCCAGTCAGTAGCACCACA
AGGGTTAGAACCCTGGCCAGACATCCACCCAATTTCACGGGTGAA
CAGTTAACAGCCTCTGAGAGTCTTGTCCTCTCTGGGCCTCCGTAA
CACC

In some embodiments, the intronic domain that promotes trans-splicing primarily in the brain comprises or consists of sequences from the gene CLPTM1. In some embodiments, the sequences from gene CLPTM1 comprise or consist of:

(SEQ ID NO: 444)
TTTGGACCACTGAGGGAACGCGCATGCGTGCTAGGCAGGGCAGTC
CGAAGGCTTGGGGGTGTATCATAGGGTCTCATCGGAGGTTTGTTG
GCCGTAAGACCCCTGGAATTGGAACTCTAAAAACACAGAAAGGAT
ATGAGCTCAGGAGACTTGAACGAAAGAGTTGTCTTAGGAAGAGGG
GTCAGAAGACAAGGGTGCTGGGCAAAAGGCTAGTTCTTCGAATCA
GAATACGGTGGCCAGAAAGGTTCCTCTGACGAAAGAGGGGCGTGG
TTCGAAGCCGTACAGTGGCCGGTAAAGCTCAAGAAACATGGAACG
AAAAGGACGCGAAGAATCGGCAGGGAGAAGCGGACAGGATGTCCC
GAAAGGCTCATATAACCGAAAAGGCGGGTTAATGTGAAGGGACGG
AAGGGACAGAACTTGCGGGAGGCACATGCTGGCCGAGAAAGTCCT
GGAGTTCGGAGCATACAGTGTTTTTATTAGGTGGAAACAAACGGG
CTGG

In some embodiments, the intronic domain that promotes trans-splicing primarily in the brain comprises or consists of sequences from the gene SLC1A5. In some embodiments, the sequences from gene SLC1A5 comprise or consist of:

(SEQ ID NO: 445)
GTATCTAATAACCCTCTGTGTGTCTGTCTCTGCAGAAATATCTTC
CCTTCCAACCTGGTGTCAGCAGCCTTTCGCTCAGTGAGTCCTCAG
GGGTGTCCTGAGGGGATGGGGGTGGACAGGGAGGGATGCCAGCCA
GGGGGAGCCCTTGCTACCTGATTTTGGTTAGTTCCTGGAAAAATG
CATGTCAATGAGAATACGGGCTCAGAGCCTCCTGTGAGTTTTAAT
TCTAGCTGTGTGACTCTGGGTAAATGTGTTTACCTCTCTGAGCCT
CGGTTTTCCACCTCTGGAAAATGGGGGTATTGATCATCCTGCCCC
ATGGGGTTGCCAGCAGATGAAATGAGATCACATTGAAAGGATGTG
GAAGGGCAGGCTTTGAGCATTGTTAGCTTGGCATGGGGTTGGGAG
GCCCTGGTAACGATGGACACTGACTCTTCCCAACCCCACCTACAG
TACTCTACCACCTATGAAGAGAGGAATATCACCGGAACCAGGGTG
AAGG

In some embodiments, the intronic domain that promotes trans-splicing primarily in the brain comprises or consists of sequences from the gene SLC1A5. In some embodiments, the sequences from gene SLC1A5 comprise or consist of:

(SEQ ID NO: 446)
GAGAGAGTCTCACTCGGTCCCAGGCTGGAATGCAGTGGCTTGATC
TTGGTTCACTGCAGCCTCAACCTCCTCGGGCTTGAGTGATCCTCC
CACCTCAGCCTCCAGAGTAGCTGGAACTACAGGCACATGCCACCA
TGCCTGGCTAATTTTTGTATTTTTTGTAGAGACACGGTTTCGTCA
TGTTGCCCAGGTTGGTCTTCAACTCCTGGCTCAAGCGATCTGTCT
GCCTTGGCCTCCCAAAGTGCTGGGATTCCAGGCAAGAGCCATGGC
GCCTTGGCCTTAGCTACTATTTTCATCTTAAGGCAGACACTGTCA
CACAAGAGTAAGGGTAGCCTTGTAAACACAGATGCATATTCATGA
GATATGAAGCCGTCCCTTTTCTCCCTCCCCGCCACCGCCTCCCAA
CACACCCAGATCCTCCCCCATACTCTCAGAGCCGCTGGCAGGCCC
TCCAGGAGGAAGTGGCTGATGCAAGCTGTCCAGGGTATTAAGTTT
GCTG

In some embodiments, the intronic domain that promotes trans-splicing primarily in the brain comprises or consists of sequences from the gene RPL13A. In some embodiments, the sequences from gene RPL13A comprise or consist of:

(SEQ ID NO: 447)
GAGAGGCTGAGGCAGGAGGATCCCTTGAGCCCAGGAAGTGGAGGC
TGCGGTGAGCCATGATCGCCCCACAGCACTCTGGTGTGGACAACA
CAGCGAGACCCTGTCTCACAAAATAAAGTAAGCCCGGACTGAGTG
CGGAAAGGCGGGCCTGGCGGGTCTGGTCTCCCCATGCGGGCCACC
AGAGGCCCTGCAGCCTTCAGTCGCTTGAAGGGGTAATGGCGCTTC
CACTCACAAACATGGCGGACAGAGCGTGTGAACGAGATGAACAGC
CCCTCAAAAATATGGCCGCCGAGGCTGGACGGCCGTGCCCCAGCA
GCACCGCCTCCGCGCCCCACGTGATCTCTCGCCGGGCACAGCGCT
GACCGCGGAGGTCCAACCGGAAGAATGTCCGGATTGGACATTCGG
AAGAGGGCCCGCCTTCCCTGGGGAATCTCTGCGCACGCGCAGAAC
GCTTCGACCAATGAAAACACAGGAAGCCGTCCGCGCAACCGCGTT
GCGT

In some embodiments, the intronic domain that promotes trans-splicing primarily in the brain comprises or consists of sequences from the gene RPL13A. In some embodiments, the sequences from gene RPL13A comprise or consist of:

(SEQ ID NO: 448)
TTTTCCAAGCGGCTGCCGAAGATGGCGGAGGTGCAGGTATGGGCT
CCGCGCGGGCCGGGGCGGCAAGGGGCCGGGTGGGATCCAGGCCGG
AATGGGGTCGCACCCTCTCTGTCTTGCATGTTTTGCGGAGCTTAA
CATCCATAATGAAGCAAAATGGAAGTCTTTTGGGATAAGGGGAAT
CTGTAGGAAATGCCGCGGCTCTCTTTAGGCCTAGGGTTGGCTACA
ATGTGGCATTTCCTTCTCGAGGCGAGGGTGATAGAGCTTCCAGCA
CAGGACAGGTATTTTGCAACTAGATTTTGCTTACCTTGAACTCGG
GTAGTGGGTGGGGGCCCTGGGGTAGAGTCTTGGCCGAATTGGACC
CTTGTGTGTCCTCAGCGATGAGGAACACGCGGAAGTGTGGCCGGG
CGGTCTCGGGGCAATGAGAAACTGAGTTTTGGCCAAAATGGAAGG
TATTTTTATCCCGCTCAGTGCGCGGGCTACTTTGATTTCACGTGG
AGGT

In some embodiments, the intronic domain that promotes trans-splicing primarily in the brain comprises or consists of sequences from the gene NOSIP. In some embodiments, the sequences from gene NOSIP comprise or consist of:

(SEQ ID NO: 449)
GTATGCACCACCACGCCCAGCTAATTTTGTATTTTTAGTAGAGAT
GGGGTTTCTCCATGTTGGTCAGGCTGGTCTCGAATTCCCAACCTC
AGGTGATCCGCCCACCTCGGCCTCCCAAAGTGCTGGGATTACAGG
TGTGAGCCACCTCACCTGGCATTTTTTTTTTTTTCCCCAGAGACA
AGGTCTCACTCTGTCACTCAGTCTGGAGTGCAGTGGTGCAGTGGC
ACGATCATAACTCACTGCAGCCTCAACCTCCTGGGATCAAGTGAT
CCTCCTGCCTCAGCCTCCCAAGTAGCTGGGACCACAGGCGCATGC
CACTATGCCCGGCTAATTTTTTAAATTTTGTTTTGTGGAGATGGA
GTCTTGCTATGTTGCCCATAGCAAGTTCAAGTCTGGTCTCAAAAA
CCATGCTGCTGCTGCTTTTTTTTGGTAGAGACAGGGTTTTGGCAT
GTTGCTCAGGCTGGTCTTGAACTCGTGAGTTCAAGTGATCCTCCT
GCCT

In some embodiments, the intronic domain that promotes trans-splicing primarily in the brain comprises or consists of sequences from the gene NOSIP. In some embodiments, the sequences from gene NOSIP comprise or consist of:

(SEQ ID NO: 450)
GCCATGTTGGCCAGGATGGTCTCGATTTCTTGACCTCATGATCCG
CCCGCTTTGGCCTCCCAAAGTGCTGGGATTACAGGCATGAGCCAC
CGCGCCCCGCCTGCATGGATGTATGTTTTTATTTCTATTGGCAGT
GGAATTGCTGGGTCAAAAGGTGACTATTTAACCTTTTGAGAAACT
GTGACTTTTCCAGAATGGCTGTACCATTTTACATTTCTACCACCA
ATGTATAAGGGTTCCAGTTTCTCCATATCCACACCAGCACTTATT
ATTTATCTTTTTGATTATAGTCATTCTAGGGGGTGTGAACTGGTG
TCTTTTAACATGCTTATTGGCCATTTGTAGATCTTTGAAGAACTG
TCTATTCCAATCCTTTACCCATTTTCAAATTGTGTTAGGTGTCCT
TTCATTATTGAGTTGTAAGTTTTTAATATATTCTCACTGCAATTC
CCTTATCAGATATCCTGATTTTTTTTTTTTTTTTTTTTTTTTTGA
GACA

In some embodiments, the intronic domain that promotes trans-splicing primarily in the brain comprises or consists of sequences from the gene NUP62. In some embodiments, the sequences from gene NUP62 comprise or consist of:

(SEQ ID NO: 451)
AAACCCACCTTGTCCCATCCACATCAGGACATCCCAGCTGGAGTT
CAACCTTCATCCCTTCTGTGGCAGTTAGGAGACTGAATCAAGGTC
CAGAGAAGGTGGAGGAATCCTGATACTGAGCGGTACACACAAGCT
TCTTTCTCCTTCCTTACCTGTTGCGGGCAGGAAGTCCTTCTTGGG
ATCTGACCACCTTCCCTTAGAGCACTTACTGTCCACTCTGCCAGT
TTTTGAGCACTTGCCCTATATTTTCTTGGGCCTATAGGGGCTGTG
TTATCACTGAGGCAGTATACAGAGTAGCAGCTAAGAGCATAGACT
GAGCTTGTCCCACCCGTGGGCCTGCATGCGGCCTAGAACAGCTTT
GAATGCAGCCTAACACAAATTTGTAAACCTTACCAAAACATGCGA
ATTTTTTGCGATTTTTTTTTAAGTTCATCAGCTATCAATAGTGTA
TGTTATGTGTGGCCTAAGACAATTCTTCAATGTGGCCCCAGGAAG
CCAA

In some embodiments, the intronic domain that promotes trans-splicing primarily in the brain comprises or consists of sequences from the gene NUP62. In some embodiments, the sequences from gene NUP62 comprise or consist of:

(SEQ ID NO: 452)
AGACGGGGTTTCACTGTGTTAGCCAGGATGGTCTCAATTTCCTGG
CCTCGTGATCCGCCCGCCTCGGCCTCCCAAAGTGCTGGGATTACA
GGCGTGAGCCATCGCTCCCGGCCTCAGCATATTTTATTAATAGGA
ACTCTGGGGCCCAGTCTCATTTCTTCCTTACCACTTGTAAGAGCC
GCAGACGTTGGACTGTCCAACACCTGCTGTCATTCTTCTGTCTCC
CTCATCCCTCTAAAGTGTCTGTTTCCAGCTATCCTCATCTTCCAT
TCCAAATGATGGGTGCCCAGGCCCCATCCCCTGGCAGGGATTCAG
ACATCGAGGCCCCAGCTGCGTCCTTTTCTCCATCCCTTGCCCTTG
GCTTCCTTTCAAACCGTCCTTCAGCTTCCACTGGATAGGAACTAT
CTCCCAACCCTGCCAAAAATGGAAGATCTCCGTTTTCGTTTAGTC
ACAGGGTAGCCATCTTGCCCCTTCTCATGCACTGGGCCCTCCTCC
ACCA

In some embodiments, the intronic domain that promotes trans-splicing primarily in the brain comprises or consists of sequences from the gene ATF5. In some embodiments, the sequences from gene ATF5 comprise or consist of:

(SEQ ID NO: 453)
GGCCCCAGAGTTCCTATTAATAAAATATGCTGAGGCCGGGAGCGA
TGGCTCACGCCTGTAATCCCAGCACTTTGGGAGGCCGAGGCGGGC
GGATCACGAGGCCAGGAAATTGAGACCATCCTGGCTAACACAGTG
AAACCCCGTCTCTACTAAAAATACAAAAAATTAGCCGGGCGTGGT
GGCGGGCGCCTGTAGTCCCAGCTACTCGGGAGGCTGAGGCAGGAG
AATGGCGTGAACCTGGGAGGCGGAGTTTGCAGTGAGCCGAGATCG
CGCCACTGCACTCCAGCCTGGGCGACAGAGACGGAGAGACTCCGT
CTCAAAAAAAAAAAACAGTTGAACAGGAATGTATGAGTTGAGCTC
TGTGCATAAGGCTTGCCAGACATCGTCTTATTTGAGTCTCATAAA
CCCACCAGGCGAGTTGATTTTTCTTATTTTACAGATAGGGAAATG
AAGTAAGAGAACTTAAGTTATTTGCCCGAGGCCATCCTGGCAGGT
CTGT

In some embodiments, the intronic domain that promotes trans-splicing primarily in the brain comprises or consists of sequences from the gene ATF5. In some embodiments, the sequences from gene ATF5 comprise or consist of:

(SEQ ID NO: 454)
TCGGATCCCCAGCTGAGAGGGAGGAGGGTCCCGGACCCTAGGAGT
GGGAAGGAAAGGCTCGGATCCCCTGATCCCCAGGAGGAGGGGACC
CGGCTGCCTCCCGGTTGGGGCCGCGCGAGGGCGGGGCGCGGAAGG
ATCCGGGAGGGCCGTGCTCCGCCACCCAGTATATATCTGTCCCCA
GTCCCCGGGGCCGCCTCATTCCCTGTCCTCGGATCACAGTCTCTT
CTCACTACAGTGTCGCCGCCTCTGCCTGCGTAGCCCCGGCCATGG
CTCTGTAGCCTCGACCCCTTTGTGCCCCCGGCCCGTCTCCGCGCT
CACCACGCCTGCGCTCTCCGCTCCCACCTTCTTTCTTCAGCCGAG
GCCGCCGCCGCCTCTCCTTGCTGCAGCCATGGAGTGAGTAACCGC
TTACCTCTTCTCCAACTGCCTTTCCCGCCCTTTCGCAGCTCGTGG
CCCTCTTTAGGGAGCCCTGGGGGGTGGGGAGTCGCGAGCGGGGAA
TCAC

In some embodiments, the intronic domain that promotes trans-splicing primarily in the brain comprises or consists of sequences from the gene PTPRA. In some embodiments, the sequences from gene PTPRA comprise or consist of:

(SEQ ID NO: 455)
GTTTTCTTACTTTGTCAATTTCTAATTTTATTGCATTGGGACAAA
AGAATATTGTACTCTTTCTACTGTTGGGGTTTATAAGGGCTGTGG
ATATTTCACTCGCCTTTGAAAAGAAGGTTTTCTCTGTTAGTCTGT
AGAGTTTGGTATGTACCAATTAGATTTTATTACTTATCATTTTGG
TCTTTTGTATCCTTACTTAATTTTGTCCTCTTGAATTTTAATGGA
GCAAAAGACATAAAGTCCTCTAATAACATGCGTTCTGTTTGCATT
CTCATACTTTTTATGAATATTGATGCTGCACTATTTGTGTACCCA
GGGAGAAGGCCAGACCACTGTCCAAAGTTTAGTGAATCTGGGCAG
CCTTGTTTCCCAGTTGTTGGAGGATGCCTCATGGAGGAAAGCATT
CCTAATCCTGGAGCTTGTTTTGTTGTACTCTAATTGAATTGTAAT
GTGTTTCTTTAACCTGAATGAATGTTTCTATTTTTTACTTATTAC
ACAG

In some embodiments, the intronic domain that promotes trans-splicing primarily in the brain comprises or consists of sequences from the gene PTPRA. In some embodiments, the sequences from gene PTPRA comprise or consist of:

(SEQ ID NO: 456)
TGAGCTGCTCACCTTATATCTGTTGTTCCTTTTACACAGTGTACA
GTATTCATTTATTTCCTCTGCTCACAGTCTGTGGTAACCGTGTGC
ATCTGTGGCTGTGTTGTTTGTTTACTTTCCCTTAAGTTATTTCCA
TGTTAATCTCATGGAGAAGAGCAATAGAAACAAGTACTGTATTCA
GTATGTTTTTTAATATAGACTATGGATTCTAACAGCTATGATGTA
TTTTAACAAGTAACAAAATATATCTTACTTTGACATGTCACTTTG
TTAACATTACTTTTTGGTGATATTAGGTCATAATTTCTATACCAT
TAGTTACTTCTGATTTCTAGGCCACAGTTCCCTTTAAATATTCTT
TGTGTTGTTTTTCCCCTAGTGTATAAAATGTCAACCCTTTGTGGC
TTTATATGGATTTTATGGATTTTCAGCCCTTAAATGTAAAGTCTC
TATGGCCTGAGATGTTGTGTCTGTGGTTTAAGCTGGACTGCTGAG
TCCC

In some embodiments, the intronic domain that promotes trans-splicing primarily in the brain comprises or consists of sequences from the gene TPX2. In some embodiments, the sequences from gene TPX2 comprise or consist of:

(SEQ ID NO: 457)
AAAGAACAGACCTTCTTAATTCGAATCCCATCTTCTCCATTTATT
ATGTGATTTTGGACATGTTTGTTCAACTTCTCCTAGACTTTGTTT
TCTTTTTACGACATGGGGATCATAAAGCCGGGATGCAGTTGGGAA
TAAAACCGGATAAAGCTCCCAACGAACCGCGGGTTTCATAAGATC
AGGGCAGGGACTGGGTAAAAGGTTTCAAAGACTCCTGCACTATTG
GGAAGGACGCTACGGGCGAGTCTATGTTGCATGCAGGCGACTCCA
TGTTAAGACGAAGTGAACTCCCTGAACCTTCAAGGGATAATGCAC
AACCATAACGAGAATAATTTTTAAAAATAGAGTTTTCTTCCCTGA
AAACTTGGAGAGGTCCAGGATGCGCGCCATTCTGTGATCTAGGTT
GACTCTCCACCTCTTCAGTTGGTACCACCTTTCCCAAATAGTTTG
GAGCAGAGCACTCCGGTCTTTGTAATAACGTACTCCTAAGAGCTG
TTCC

In some embodiments, the intronic domain that promotes trans-splicing primarily in the brain comprises or consists of sequences from the gene TPX2. In some embodiments, the sequences from gene TPX2 comprise or consist of:

(SEQ ID NO: 458)
TAACGATAATTATTGTTATTACCATGCTAACAGGTTATAAACATT
GACTGAACTAAGCACCCTGCATGCCCGTTCTCATTTAATTTCCTC
GCCAAGCCAATGTAGTTGGTGCTATTCTTGGTCCCTCTTGACAGA
AACTGAGGAAGGAGGAAGGAGGAGGAAACTGAGGCCCAGAATCTA
GATAAATTTCGAGAAATGGGTGATACAGTAGCCATGCCCTCAGGA
AGTGTGGCGTTTGATGGAGGAAAACAGAGGTAGTCACAAATAAGT
ACATTTCAAAACTGTTGTCTAAAACAGGAAGATAATTAGATGGTG
TGAGAACATAATTGAGGGAGAGTTTAAGTCCACTTGGAGTTGTCT
TCTATTTGGGATTTAAAATTCATTATATGTTATCCATATTTGTCC
AGCTCTCTTTTTGTATGGAATCACCAGATATTATTCCCTTTCTCA
TTTCCTTTCTCAACATTTATAGATTGGAGGGGGGAAGGAAAACCA
AACC

In some embodiments, the intronic domain that promotes trans-splicing primarily in the brain comprises or consists of sequences from the gene CBFA2T2. In some embodiments, the sequences from gene CBFA2T2 comprise or consist of:

(SEQ ID NO: 459)
CAAGTCTGGATCTGAGAGGCAGCTTGTGTGAATCATTTACATACA
TTTCTGTTTATATTCCCCAAATTAATTTGCAACTTTATATTTTTC
ATCCAATTATATGTTCAAAACATATTTTTAATAATTGATTTTAAG
AAGGAATTTGTTATATTAGGATTTAAAAACTGGCTCAAAAAACAG
AAGCTATTAAACAATAGCAGAAACCAGACAGTATATAGGACTTTA
AATGGTAATTGCTAAACATTTGAATGTTCCCAAAAATGTGAGTCT
GTGTGTCTCTGTGTGTCTCTACGTGTTATTCCACTAGGGATTTGC
ACAGATAGGTTTTGCACAATATCCTTGCTGTTATTGTCAGCCATA
AACAAATTACACGTATTTAACAAGTTAATGTCAGATTGGCCAGGA
GCCACTGGGAGGTGTGTACTTAGTGACATCAGCTGGCAAAGAGCC
CTGGTAACAGGGAGGGTTGGAAGGGTAGTGGAGAGAGAGGAAGCA
GGAG

In some embodiments, the intronic domain that promotes trans-splicing primarily in the brain comprises or consists of sequences from the gene CBFA2T2. In some embodiments, the sequences from gene CBFA2T2 comprise or consist of:

(SEQ ID NO: 460)
TAAGTACATTTTAATATAGTTTTGGGGGTTAAAGTGCACAATTCT
CTGCCTTTCAAAGCTTTTTACTTCTTTCAAATTACGATGATATGA
ATGTTTCTTGGTATTTGATACAAGAGTTCAGCCTTTCTACTCAGT
TTTATTTTAATGCTTCATTTTATTTATGTTTCTATTTTTCTGTTG
CCAACCTTATATTTGAATGTCATGCTTTTTTGAATGCAAGGTTTA
CTTAATAATTAGATGTAGTATATGAATTCTACAGCTAGAAAAGTA
GTCCCACAAATATTTCTGTAAGGTTTTCCACGTTGGATTGTTCTG
TGATGTTTAGAGGGAATTTTTTTCCCCTTTAGTATGCATAATACA
AATAGGCCATATTAAAAAGATGGCTCATAAAACGCTCTTTTCTAT
TGTCAGATTTTATTTCAGTATATGCAGGCAAATTATAACTAAAAG
AGGTTGAAGCAAAACTAACAGCATCTCTTCGTTTTAGATATATCT
AACA

In some embodiments, the intronic domain that promotes trans-splicing primarily in the brain comprises or consists of sequences from the gene DYNLRB1. In some embodiments, the sequences from gene DYNLRB1 comprise or consist of:

(SEQ ID NO: 461)
AGCAGAGTTCTCGCAATTCCTACTTAAGCAGAATGAAAGCCATCA
AGGGCAACAAAGTGGAGGTGCTGTGTCCTTGGATAGAATGTTGAT
TTCCAGCCAGGCGTGGTGGCTCACGCCTGTAATCTCAGCCCTTTG
GGAGGCCGAGGCGGGCGTATCACCTGAGGTCAGGAGTTCGAGACC
AGCCTGGCCAACATGGTGAAACCTTGTCTCTACTAAAAATACAAA
AATTAGCCTGGTGTGGTGGCGGGCACCTGTAATCCCAGCTATTCG
GGAGGCTGAGGCAGGAGAATCGCTTGAACCAAGGAGGCAGAGGTT
GCAATGAGCCGAGATCACACCACTGCACTCCAGCCTGGACGACAA
GAATGAAACTCGGTCTCAAAAAAAAAAAAAAAATGTTGATTTCCA
AGTCAGCAGAGCCCTGGCAAGCACAGAGCTAGACAGAACGTGCTC
CCCAGCCCTGTCTAGTTCTGATTAAACCATGTGGTCATTTCATTT
ACAG

In some embodiments, the intronic domain that promotes trans-splicing primarily in the brain comprises or consists of sequences from the gene DYNLRB1. In some embodiments, the sequences from gene DYNLRB1 comprise or consist of:

(SEQ ID NO: 462)
TGAGTGCTGCTTTTTTGTATACTCATGGGGCTGATTGCACCATAG
CTGAGAACGTGGAATGCAGAATGAGCCCACCTGGCTTTGAATCTG
GGCCCTACTTAACTTATCAGCTATGTGGTTTTGGGCATAAGCCAC
TTTCTCTGTTAAGTTTTCTCATTTGTAAAATGTGGGTCATAATTC
TGCCTTCCTCTAGGGGAAGCTTTTAGAATAGTGCCTGGCATGAAA
TGCCTATGAATGTCTCAGTTAGTGTGTGTGTGTCTTTAACTTGCG
TCCCATTCTCAGGGCCCCCACATCAGTGTCTCGTTTACATCTCAC
ACAGTGACCCTGCGAGGCAAGGGTTTGCCCAGTTTGCAGATGAGA
AACCCAAAGCTGAGAGAGGTGTGGGGAACCGCCCAAGGTCACCCA
GCTGTGAATGAACGTGGTCAGAACACAGAATCTGAGTTGGTCACA
CTTCCCACTGATCCATGGGGCCTTTAAGCCCTCTGGAAGCTTCCA
TTAA

In some embodiments, the intronic domain that promotes trans-splicing primarily in the brain comprises or consists of sequences from the gene ERGIC3. In some embodiments, the sequences from gene ERGIC3 comprise or consist of:

(SEQ ID NO: 463)
CGGGGGTGCTCTGTATTTGGAAGGATGTATAAATGCAAAGGTACA
GTTGCCCAAGGCCAGAGATCTGGCTGAATTGAGGCCTGGGAGTGA
GTTTCTAACATCCTTGTTGATTGGGGCTGGGGTGGGTAATAATCT
CTTACAGTGTAAAGTTTACAAGTATTTTTCCATCTTAGATCCCAC
GTGATCCTTAGGACAGCCCTTTGCAAAGGAGAACACTGAGGCCCA
GAAAGGGCAACTGATTTGCCCAAGGCCACACAGTCAGGAACTTGG
CTCTTTGAGCTCTAGGCCTCTTGTCCTTCTAGGCCACTATTTTGC
TCCTCTCTGGCCAGATTAGTTCACTCTCGAGGAGGGGCCCAGAAG
GAGGCTTGTTAGCTGGGGCCAGACTGTGTTGCTGAGGCTGAATGT
TCCAGTCTTCCCTCCTGCAACATATCTTGCTCTCATAGTTCTCAT
TGTGAAGCTCTGAATCTTTCTTCTCTCTCCTCCCTCCCCCATGCT
GCAG

In some embodiments, the intronic domain that promotes trans-splicing primarily in the brain comprises or consists of sequences from the gene ERGIC3. In some embodiments, the sequences from gene ERGIC3 comprise or consist of:

(SEQ ID NO: 464)
TAAGTGGCCCTGCCCCCATGGAGGCCAAACCCCTACTTCTGGGGC
AGCCTGCCCTACTAGAATGGCGGGGAGGTTGGATGGGGCTGTGAT
TGGGTCTCCTCTGACCTGGCTGCTGGGTGACTGTAAAAGGTCTGG
GATGTATCTTTGAAGTATGGTTCCTTGCAGCAGGAGGGATTGAGG
CTAGAGGGCAGGAATAAATTGTTCTGGAACAGTTGGTACAGGGTC
CCCATGGGATTTCTTCTGGGGAGCTTTGAGGGTGGAGGCAGGGAA
AGTGTGGATGGCATTCTCAGATCCCACCTCACTTTGAGGTTCAGT
GATTTGCCATCAGCTCTAGTTAGTGAGAGCCCATAGTCGGTGGGT
TTTGTTTCTGGAGATGCAGGCAGGGCCATGGTGGTAGTCAAGGCA
TAGAGTTCCCTGTGGACGTCAGGGAGGAACCAGGGTCTGTTCTAA
AGACTCCTGAGGAGGGCGTGAACTCTCACACAGGCCCAAGGCTTG
CCCT

In some embodiments, the intronic domain that promotes trans-splicing primarily in the brain comprises or consists of sequences from the gene DLGAP4. In some embodiments, the sequences from gene DLGAP4 comprise or consist of:

(SEQ ID NO: 465)
GGCACCCATTCCTAGAGGCAGGAGCCCCAAGTTCTCGGGGGTGGG
GTGGTTCCTGACTGGCGCGGTGTGACCCGGGCCAGTCTCGTTCTC
TCTGGGCCTCAGTTTCCCTCCTCCACGGGGCGGCGATGGTCAACA
CAGGTTCCTGCCGGCCCGCCCCGTAGCCTGGGGTTCCAGGCTCCC
GTGCCACCGCGGCCCGCGCTGTGTGACCTTGTTCCAGTAGCCTGC
CCTCTCTGGACTGGCGGCCCCGGATTTCCAAGCGCTCTTCCGGAT
CGCGACAGAGCGGTCGCCCGCTGCGGAGACCCTCCCCCTTCCCCC
GGGTTGCGGGGGCGTGGGTGGGGGTGGGGCGCCAGGAACGGCGCG
GGCCCCCCCAAGGCCCCCCCAGCCCCGCGCCCGCGGCCCGATCCG
CGCCTGGTTTTGGGCGCAGCCGCACGGCCCCCTCCCCCGCGCTCC
TCTCCCCTCTCCCCGCCCCCTCCCCGCCGCTCTCGGGGAGCCGGG
GCCG

In some embodiments, the intronic domain that promotes trans-splicing primarily in the brain comprises or consists of sequences from the gene DLGAP4. In some embodiments, the sequences from gene DLGAP4 comprise or consist of:

(SEQ ID NO: 466)
TGAGGGCGCGGGGGTGGCGCGCGGCGGCTCCTACCCCCTTCCCGG
GCCGGCGCGCTCCGTCCCTCGGCTAGACCCCCCGATCTGGTCTCC
TCCCCGACAGACCTCCCTGAACCGGGGGACGGCGCTGGCGCAGAA
AAGCCCCCTCCCCTGCCAGTTCTGTCTTCCGATCCCGGGTCCGAG
GGGCCGGCTGTGCGGCTGGGAAGCGCCTTCTCCGGTTGGGATCGC
CCCATCCATGTCTCCCCGGACCCTCAAATCGGGGGCGGCGGCACC
GGGGCCCGGAACCCCTGTCCGGGACCGGATCCCCATTCCGGCTCT
TTTTCCCGCGGACTCCCCCGTACCCCATATCAAGCGGCTGCCAAA
GCCTGGAAGCCCCCTCCTCAGGCCCGCCCCCTAATCCGCAGGGCG
GCCTGGGGGTCAGGCGGACCCCTCCCGTGCCCGGCCAACCTTGGC
GCTTGGGAGAGCAGGGCTATGCCGGCCCGGGCATTCCTCCATTCA
ACCC

In some embodiments, the intronic domain that promotes trans-splicing primarily in the brain comprises or consists of sequences from the gene SOGA1. In some embodiments, the sequences from gene SOGA1 comprise or consist of:

(SEQ ID NO: 467)
GTGAGGCCCACCCCTGCCAAGCCGCTTCCACCCGCAAGGGAGAGT
TGCTACAGAAATGATTCAGAAGCAGGAACTCCTTCAGGTATAGCT
GGATCTAGGTGCTCATCCAGTGAGGTCTGGCACACCATCTCCTTC
AGGTATAGCTGGATCCAGGCACTTAGAGAATGCAGTGGGAACTAG
ATTTTTCTTTCTGCTTCAGCCTGTCAGGCCTGCTTTCCTCTGTGT
TAGCTTTACTCTTAAACAAGCTATTCCCAAATATGGCCACTGTTA
GTTTCATGCTCATATCTTACAGCTTAGCAAGTACAGTGGATTGGC
CAGATATGAGTCATGTGCCCACCCCTGGAGCCTACCCAAACCCAG
GAACCAAGAGGGAGGAACTAAGAGTGGTTCCCATGGGAAAATTGG
GAGGCCTCCAATCAAAAGAAGGAGGAAAGGAAAAGCAATCATTGA
CCCTCACTCCCCATTATAGTTCACTTCATGCAGCCAGGCACCACC
TACC

In some embodiments, the intronic domain that promotes trans-splicing primarily in the brain comprises or consists of sequences from the gene SOGA1. In some embodiments, the sequences from gene SOGA1 comprise or consist of:

(SEQ ID NO: 468)
CCTAACCCTCTTCCAGATCTCACAGCTCCCTCCATCTGCATATTC
CACGGTGTCCCCTGCACCTGCTGCCCCCAAAGACCTGGGCATTTC
AGGTGCTCCTCCCACCAAGAACCTGACCAGCTCATGGGGGTTAAT
GGGGGGCATTGAGTCTAAAGGTTGCACATTCCCTTTCCTGCCACT
CTGAGGTTTGAGGCCAAGAAAACCATCTTGCTGGAATGACCACTA
GTAATCTTTCTGAGTTTTTATGAGGATACTGACATCATGCCTGAA
AAGCCCACAGAAGAGCATGGCTTATAGAAAGAGCTCTTCAAACCC
TTATTGTGTGGAGGGGTTAGGGGTTAGTTCTGAGTGGGGAGGACT
AAGCTGGGTTAGTCCCTAGTGAGGGGTCTTCTGAGGGGACCTCTC
CCCTTTTCCCCGCTGGCACCCAGGAGGGAAGGAGAGAGAAGGGCC
AAGAGGAGTGGCCTCCTGCTGCCAGCTGCTTAGTCTGCTTCTTTC
CGCA

In some embodiments, the intronic domain that promotes trans-splicing primarily in the brain comprises or consists of sequences from the gene DBNDD2. In some embodiments, the sequences from gene DBNDD2 comprise or consist of:

(SEQ ID NO: 469)
CCTTAGGGGCAGAGCCTAGTCTGGAAGTCAGGCCTGTGACTTCAG
AGCAAGGGTTTGGGGTAGATGGCACATGCGTGTGTACGTGTTTGT
GTGGGTGGAGAGAAAAGGAGGGGCGCGGGAGGAGTTGCTTTAGCC
TTTGTCTGGGTTTTGAAGGATGGGAAGGGGTGGGCTGCAGCACTG
GAGGAAGGGAACCCTCCACCCTGAGATCTCTGTCTCTATCCTATC
CTGTCCCTGGCCTTCTGAGGCAAGCGGGGCCAATTAAGGGGAAAA
CGTACCTCCTCCATTTGTGCTGAACCAATCCCTCCAACCCCTCTC
AGGAGGGCATGATATGGAGAGTTGGGCATTGGCTGTGTTCCCTGA
ATACAGAGTATCTCTCTTGTGGTGCCTGGAACTGGCATCCCCTTT
GTGGAGCTTAGGGCAAGCCCCGCCTCTGCATGAGACTTGGTTTGT
GGGACACACTTGGTTTCAGGGAAGGGGAAAGAGGTCACCAAGGGC
AGAG

In some embodiments, the intronic domain that promotes trans-splicing primarily in the brain comprises or consists of sequences from the gene DBNDD2. In some embodiments, the sequences from gene DBNDD2 comprise or consist of:

(SEQ ID NO: 470)
AGCTGACATGGACCCAAATCCTCGGGCCGCCCTGGAGCGCCAGCA
GCTCCGCCTTCGGGAGCGGCAAAAATTCTTCGAGGACATTTTACA
GCCAGAGACAGAGTTTGTCTTTCCTCTGTCCCATCTGCATCTCGA
GTCGCAGAGACGTAAGTCCCAAGTCCTGAGAAGAGGGACTGGGGT
AGGGTAGGGAGGATGTCCTGTGGGTCCTGAATCTTGTGGCACTCT
CTCCCCTCTGGTTTTCTTGGCCCTCTATGCTTCTAACTTGGGACC
TGACATGTAACTCTCACTGTCCTGGTGTGCAGCTTGGGTCCTCCT
GACTTGCCCACTTCTTGATCCGCAGCCCCCATAGGTAGTATCTCA
TCCATGGAAGTGAATGTGGACACACTGGAGCAAGTAGAACTTATT
GACCTTGGGGACCCGGATGCAGCAGATGTGTTCTTGCCTTGCGAA
GATCCTCCACCAACCCCCCAGTCGTCTGGTATGCCCCTCTGCTTT
GGGG

In some embodiments, the intronic domain that promotes trans-splicing primarily in the brain comprises or consists of sequences from the gene TPD52L2. In some embodiments, the sequences from gene TPD52L2 comprise or consist of:

(SEQ ID NO: 471)
AAGGGTGTCCCACATCCTCCCACGGTGCGTGTGCTCCCTCAGCTG
ACGTGATTTGGGGAACACGGGCCGCTCTCGTTCTGTGGAGCTGGC
CAAGCCCAGCTGTCATGCTGTGGCACATTTCACACCTTTGCGCTG
CTGGATGGGGCTGGTTGTGCTCCAGGTGCTGTGGCTTCTCCAAAC
AGGTGTGCCTTGGGGCATAAGGCGTGGACAGGGCGTGGACCCAGG
GGCCTCTTGGGCACAGCCGTGAGGCTGAGCAGTTGCTAGTCATGA
TCTTGCGAGAGCCTCCTGTTGAGTCAGGACTTGAGGAGACTGGAG
ACAGTGCTGCTGTCCCCGGTGGTGTGCGGACTGGAGGAGCCGTCC
TGGAGGGTCTTCCCCTGCTGGGCCTGACTGAGCACGAGCAGGGGG
CCTCCCCTGGGGCTCCCCGAGGAGGGCTGTGAGTGGGTGCTGGCC
TCCCTTGCTTACACTTCGTTTCCTCTTCTCTCCTGCTGCCACCTT
CCAG

In some embodiments, the intronic domain that promotes trans-splicing primarily in the brain comprises or consists of sequences from the gene TPD52L2. In some embodiments, the sequences from gene TPD52L2 comprise or consist of:

(SEQ ID NO: 472)
TAAGGCTGAGCCTGGACACACCTGTGGAAGACTTTTCTCTGAATT
GGGGCAGGGTGGACTCCGGCAGGGCCCTTGGGCGGCTGTGCTAGT
AGAGGGCAGTGAAAGTGGGATCACCCCTTCCAGGACAGCAGTGCC
AGCCAGGTGGTTGCTGCATCTCAGGGAACAAAGCAGCAGCGTGGG
ATCGACGCAGAGGCCCCCCGGAACCCCCCGGCCCTTCTGTGTCCC
CTCCTCGTGCTTCCCTTCAGGCTCACAGACCGCCTGCTGCCGGGT
CTGTGAATAACTGGCCTGGCGTCCAGGCCTGAGTTTACTCCTGAA
AAGGAGCTTTTTTTTTTTTTTAGACGGAGTCTTGCTCTGTCGCCC
AGGTTGGAGTGCCGTGGCGCGATCTCGGCTCACTGCAAGCTCCGC
CTCCCGGGTTCACGCCATTCTCCTGCCTCAGCCTCTCCGAGTAGC
TGGGACTACAGGCGCCCGCCGCCACGCCCGGCTAATTTTTTGTAT
TTTT

In some embodiments, the intronic domain that promotes trans-splicing primarily in the brain comprises or consists of sequences from the gene CCT8. In some embodiments, the sequences from gene CCT8 comprise or consist of:

(SEQ ID NO: 473)
GTGCGTTTTGCCTCCATCCGTAAATGGGGCTAAGGCCCGTCACCC
TTAAAGGAGGTTGTGAGGGTGAAATTGAATAACGTAGATGAAATT
GTCTTGAGAACTGCGACGTCGATTATCACATAGCTCGCGAGTTGT
AGGATGGGGAAGAACGAGAACTAGCCGATCCAGAGAAGAGAGTGG
GAAAAAGGGCCGGGTCTTGGTTGCTTGCTTCCCAGTGAGAAACAT
ACGGCTTTCAGCTTAGTTGACAGAAGCCATGCGTTGTAGCCAAAT
GAGTTCCGGTCCCAACTTATGTTAGCTGGCTTAATCTAAAATGTA
ACTTTCTTGGGTTTTGTTTCCTTCGTCTTTAAATTATGGAGATTG
GACCAAGAGGTGGTGTCTTTGAGTTCTGTGTAATCTGCAGGATAA
GTGATAGAGGGGTGCAGATAACACTTTTGTTTAATGGCCTTTTCT
TGAACGTTAGTTCGGTAGCATAGTTTTAAAAAATATCTTACTGCA
CTAT

In some embodiments, the intronic domain that promotes trans-splicing primarily in the brain comprises or consists of sequences from the gene CCT8. In some embodiments, the sequences from gene CCT8 comprise or consist of:

(SEQ ID NO: 474)
TGACGTTGGTGGCTGGGTCTGGAAGATACGTGGAAATTAAGCTAA
GGATGTGTGGCTTCCAGATCAAAAACCGCAAAAATCTAACGCCGT
GACTACTGACTACGGTCAGAGAGCACAGACTGGAGCAACCTCTCA
CGGCCTGGGCTGTCTGCGCGTGCGTGAGCCAGAAACCCGAGGGGC
TCCCTGGGCCCGCCCTATCGATCGACCCGATCGGGGATCGTCAGC
TTGGTTCTGGCCACAGAGGTTGCTCTTCTCGCGATGCTTCAGACC
TGGCGGCAGGGAAAGGGTGGGCTAATTGGAGAGCCAGGAAGAGCG
TGAGGCGGCCCCACGCTGCTTTCCCAGAAGGCTGTGCGTGCTCCT
CGCTTCCTCCGCGGTCTTCCGAGCGGTCGCGTGAACTGCTTCCTG
CAGGCTGGCCATGGCGCTTCACGTTCCCAAGGCTCCGGGCTTTGC
CCAGATGCTCAAGGAGGGAGCGAAAGTAAGGGCTGAAGGAAAGGA
ATGA

In some embodiments, the intronic domain that promotes trans-splicing primarily in the brain comprises or consists of sequences from the gene TMPRSS3. In some embodiments, the sequences from gene TMPRSS3 comprise or consist of:

(SEQ ID NO: 475)
GTACATCTGGGTCTCTATGTGGTTCTGCAGCTCTTCCTTTGTTTC
AAGAGGATTTGCAATTGCTCATTGAAGCATTCTTATGATGGCTGC
TTTATAATCCTTGTCAGATATTAATAATTCCAACTCCTGATTCAT
GTTGGTGTTGGCATCAGTTGATTATCTTTTCTCATTAAAATTGTG
ATGCTCCTAGCTCTTGGTATGACAAGTGATTTTCAATTGTATCCT
GGACAATGTGGCTCTCATGTTTGCAGACTTGAGTCCTACCTACAT
TTTCAATGTTACCCTGTTTAAATGTAGTTTGTAGATCCTGGCCTA
CAGAATGAATGCACTGATGACTCTGCCAGGTAGATTGGTGGGCTG
TAGTTCCAACAATAATTTTAGAGCCTTCGTGGGCTGTGTGCTTTG
TCTGGTGCCACCTTCTCCCTGTTGGTGCCACCTTCTCCCTGTTGG
TGCCACCTGGAGGCAGAAAGAGCTTCTTGAGGCCAGACCAGCTGG
GGCC

In some embodiments, the intronic domain that promotes trans-splicing primarily in the brain comprises or consists of sequences from the gene TMPRSS3. In some embodiments, the sequences from gene TMPRSS3 comprise or consist of:

(SEQ ID NO: 476)
CATAGCTAATTCCAGTGGACCATGTTATGGCAGAGACAGGGTGAG
ACGGTGGTGCGGGAGGCTAGAGGGAATATGTTGGGGGCCAGGTAC
TGGGTCTCTTCTTAAAATCTCAGGAGTTTAGAGTGCAAGAAAACT
ACAGAAAAGACTTCCAAACACAATGGTTCTCCCTGATATTTTTAC
CCCCTATTTTGTTGCTGTTTGATGAACAGTAGTGCCTGATAGACT
CCTTGCAGTGACCAATGTTGAGTTCAGCCCTCAAATGCCAAGACG
ACCTCAGAGGTGCTGAGAGTGAGGTCTGGGAAACACATGGGGTGT
GGGTCTGTGTTTCTGTAGGGCGTGGCAGTGCAGTTGCATATACAG
CATAAACAAGCAGGGTGATGGGACCACATCTTGCCTGATAACCTC
TCCCACCATCTTCCTAGGTTTTCAGGATACCTGAGGTCAATGAGT
TCAGTGGTTGGACTTTTCCTGTGCTTCACCTCTTGCTCTTCTCAT
TTCA

In some embodiments, the intronic domain that promotes trans-splicing primarily in the brain comprises or consists of sequences from the gene PFKL. In some embodiments, the sequences from gene PFKL comprise or consist of:

(SEQ ID NO: 477)
CGGCTGTCTTTCTGGGGGAGCTTGTCTGTCTGTCTGGGGGGCTTG
GCTTTCTGGGTGTGTGTGGGATGCTTGCTGGGTTCCCGCCCCAGT
TTCAGCTGTAGCAGCCGTGCTCCCATCTATGTGGGGATGTGAGGG
AGGCACTGTTCCTTTAGAGACGGCGTTGCTTGCTCAAGTGGAGAA
GCTGCCAGTGTGGGCAGCTCCAGCTCCCTGCACGCTCTGTGCTCT
GCCCATTCTAACGTCTGTGCCCCTTCTTCTGGATGGCTGCCGGGC
TTGGCTCTGGCCCCCCTGTTGGATCCCCTGGCTGCTGGGTGGGGA
GAGGGCCACCTACTGAACCTGAGGGTGGGTCTGGGTGAGTCCTGG
CTCTGCTGCCACCTGCTGGGCTCTGGAAGGTCCCTCACCCTCCCT
CTGGGATCTCATTGGATTCCTGTAGTGTGGGGATGGTGGCAGATA
CTCTGGCTGCTACGTGAGAACAGTGTGCCGATCCCTGGGGTTTCT
CAAG

In some embodiments, the intronic domain that promotes trans-splicing primarily in the brain comprises or consists of sequences from the gene PFKL. In some embodiments, the sequences from gene PFKL comprise or consist of:

(SEQ ID NO: 478)
TCAGCACCTCCAGCCTCTGCAAGTACAGGGGGGTGGGAGGGCAGG
GGCTTTTGAGGGGCACGAGGCTTGGGGACAGAGGACCCGCATGGC
TTAGGGATGGATGGCATGGTATGGGTGCTGGGAGGGAGGCAGGAA
GAGCAGGGCTGGTCCCAGGGGCAAGCACAAACTCAGGCCTGTTAC
ACGGCCCCAGACCTCACTCAGAAGGGGACCCTGGCCGCTGGCGTG
GGTTTCCTCCTCTCTGAAGATTAGGTGGTCTTGGCTGCCCCTTTT
GTATGAAGTCATGGATATCTTTTGAGATGGGGGCTCTCGGGAAGA
AGGTGTGGGGTACATTAGCACCAGCGTTTCCTGAGTGCCGCCTCC
CCCGTTAATGTCCCTCTCCAGGAAACTGGCTTTGCCAAGGCCCCC
GCTGGGACAGACTGTTTCTTTCACTGCAGTCCTGGGAGCCGAGGG
CAAGGGGACAGGAAAGAGGAAGTGACCTCAGAGCCTGGTGGCACC
AGCA

In some embodiments, the intronic domain that promotes trans-splicing primarily in the brain comprises or consists of sequences from the gene S100B. In some embodiments, the sequences from gene S100B comprise or consist of:

(SEQ ID NO: 479)
GTGAGAAAGAGCCAGGCCAAGAGGACGCTCAGGAAGAAATGGTTC
TTTTCTTTTGGGTGGAACGGAATGGAGGGGTAGAAACTAAGTGGT
AGCTTAAAAAGCCCCTTTAGGACAATTGGCAGCATTTCAGAAGTG
TCAATAGGATGATGTGTTTTAATCTCCACATTACTGCTGCTTTGT
GGACACCTGACTGCATCAGCCCTAGACAGCTAGAGGTGTGTTTTG
CCATAAATCAGAGAAACGTCAGGTTTCATGGCCCAGAAGTGATTG
TTGACATTTTCCCAGCGGTACTACAAATACTGCCTCTTCTGTAAC
TATTTGAAGAGTAAAGATTTTGCTTCCCACTGGGGCTAAAAATGA
TGGAGAACCTAATGCTAGTTTAGTCCTATATCATAAATAAATGCT
TCCCTTTTCTCCTTATATTTCTTCCACCGCCCTGGGTAAGTAACT
CTTCAGTTTTCCAGTTTCCCTCAGTTTGAAGTGCCAGGGTCCCCA
CAGC

In some embodiments, the intronic domain that promotes trans-splicing primarily in the brain comprises or consists of sequences from the gene S100B. In some embodiments, the sequences from gene S100B comprise or consist of:

(SEQ ID NO: 480)
AGGCAGCCCCGTTGAGCCCAGGGCTGACCACAGACAAGCACTACT
GTCCACAGAACTTCACGCCCAGTGGGGCCAGACTTGGAGGATGGC
AGAGGAGAGAAGCTCCAGGGGCCTCTCCATTAGAAACCAACTTGC
AGGCCACTTAATCTCCTATGCTCAGCCTGTACTTGGAAGCTGCTT
GTTCTTGGCTGCACATTTGCTTTGTTTGAATCAATTAATCCAAGT
CTCTCTCCTCTCCCCACCCCCAACCTTGCCTTTAGGGTGACATCA
ATATTCATGTAATAAAGAAATCGCACAAAAAGCTGACTCCCCACT
TCCTGCCCTACAGGCCCTTTTTTCTTCCTCAGCCCATGTGCAATC
CTGGCTCCCAGCAAGTCCCCGGGGCTGCTTGGTCAATGCAGCCCT
GTGTGCAGGCCTGGCAGCCCTGCCACCCCCGCCTTCGGCTCCCAT
TGGCTGCCACGGCCTGCAGTGGGCTGCACCAGGGTTCATCCATCC
TCCC

In some embodiments, the intronic domain that promotes trans-splicing primarily in the brain comprises or consists of sequences from the gene RANBP1. In some embodiments, the sequences from gene RANBP1 comprise or consist of:

(SEQ ID NO: 481)
TACTGGTTTTGAATCGCGGCGCGTTTCCCGCCGCTGGGGTCAGGG
GTCGAGGTTCGGGTCGTGGGGCGGAGGGAAGAGCGGGCGGGCGGG
AGGCGCCGGCGCCAGACGCGGAGGGAAGGAGCTACGAGTAGCCGC
CGAGAGGCCGCGGAGCCAGCGACGACCGACCCAGCCGAGCCGCCG
CCGCCGCCGCGCCCCCATGGCGGCCGCCAAGGTGCCGCCGGGCCC
AAGCGGGGACAGGGTGGGCGGGCGGGGCCGGGGCCGTTATCAGCG
GCCACGTGGGCGGCCCGCGCCGCCGCCACCAACCTCCGCCGGCCT
GCAGGCTCGGCCGTCTGCTGGGCCTCGGTGGCGCGGAGTCGGGGC
GCGTGGGGTTTGGGGGCTCGAGGCCTGCGGAGTTGGGGCGTTTGG
GGGTGCAGGCTCTGCAGAGGCCTGGGGGCTGCAGGGCCCCCGCGT
GACCTTGGGGTGGGTTGGGGAGCGGCCGTCGCCACGCGGGACGGC
CCCA

In some embodiments, the intronic domain that promotes trans-splicing primarily in the brain comprises or consists of sequences from the gene RANBP1. In some embodiments, the sequences from gene RANBP1 comprise or consist of:

(SEQ ID NO: 482)
CATGTGTTCTGGAAGACTCGAACCTAAAATATGATTATTGGTGCT
CTCTTATCCTCTAAATTGGGCCAGGATTTCATGACTTTCACCGGT
ACAATTTATCTAGTTATTAACTTAGTTTTCTGAACTGTGAAAAAA
TTGCTGGCAGAGCTGTGAATCCAGTGTAAATACTAGCAGTCCTGT
AGCGACTTTTTTCGTTTTACTAAGCAAGAGCACAACTATTTCCTA
ATTAGTCAAAACTGAAAGAGTTACATTTTGCTTTAAAGTAATATT
TCAAAATCTATTTCGACCCATCATTGTGTCCTTTGATCTTCACAC
AGCAGGTATCATCTGCATTTTATGGATGAGGAAGAAACTGATGCT
CAAAGAGATGAGGCAGTTTCTGCCAGGCCACTCTCTGCAAGTGTT
GGCGGCTTGTAGGGTCCCCCATCAGTCTGTGCACAGATCTGATTG
ATGTCTGTGAATCTGGGGGAGGGGCCCCAAGTTCTGGTGTCTCAC
TCTG

In some embodiments, the intronic domain that promotes trans-splicing primarily in the brain comprises or consists of sequences from the gene UBE2L3. In some embodiments, the sequences from gene UBE2L3 comprise or consist of:

(SEQ ID NO: 483)
CCTGCCTCAGCCTCCTGAGTAGCAGAGATTACAGGCGCCCGCCAC
CACGCCTGGCTAATTTTTTGTATTTTTAGTAGAGACCGAGTTTCA
CCACGTTGGCCAGGCTGGTCTCGAACTCGTGACCGCGTGATCCAC
CCGCCTCGGTCTCCCAAAGTGCTGGGATTACAGGCGTGAGCCCCC
GCGCCTGGTCAACTATTTTATGCAATCAGTATTTATTATGCTCCT
ACTATGTGCCAGGCACTGGTTGTAACTGCCATCTGATTGGACGAT
CCGTAAACGCTGAGGCCCAGTCTGTGCGGTTCACTTGCGTTCCTC
CACGCGCCCCCCAGCACAGTGGGAAGCACACAGTAGGTGCTCCGC
TCTGCTCCTGTGCCCCGCCCCGCGGCCCCTCCCCCGCTCCAGGAA
GTGCGGGGGCTCCAGCCGCCCGGCCGGCCGCGATGCATTCTGGGG
AAGGAGCAGCACCAAATCCAAGATGGCGGCCAGCAGGAGGCTGAT
GAAG

In some embodiments, the intronic domain that promotes trans-splicing primarily in the brain comprises or consists of sequences from the gene UBE2L3. In some embodiments, the sequences from gene UBE2L3 comprise or consist of:

(SEQ ID NO: 484)
TAAGTTCCAACTCCAGGAAGGCCTGAGGTCGGCCCCGATCGCGGC
GCGGTTCTGCTTCCCTCTCCTCCACTCAGTCATCCCGATCTCCCC
TCCCTGATAAAGTGCCATTTTTGGGGGGATAACGGTTGATTTCAG
TCGTTCGAATCAAAACGGGCCAAAGTTTATAATAATAATTTGGTA
CTTTGGATCTTGAATGCAGCAGGGTGCTTTCTCGCTAATTGGCAC
CTTTTGGTGAGGAACACATGCTAAATTGGCTTAGTACTTTTGCCA
GTGGATTTGCCAAGGTCCTCTGTAGCCCACCCCGCCAGCATCTGT
GAGTCGGCGGCTGGGTAGTGCTGCCGGCCTGCAACGGGCCGGAAA
TTAAAAAAAGGCCCAGGAGCCAGTTTCCTGAGGGGAGATAAAGAG
CTGGGGTTCGTTCTAGGCGGAGCAGTGCTTGGTAGAGCTAGACAT
TGAACTTGGAGCCACAGTCTCTGAGCATTCCCTCCGTCCCTGGGA
ACGT

In some embodiments, the intronic domain that promotes trans-splicing primarily in the brain comprises or consists of sequences from the gene CSNK1E. In some embodiments, the sequences from gene CSNK1E comprise or consist of:

(SEQ ID NO: 485)
GTGATCTGAACCTGAAGTCCGAGGCCCCAAGAGTTCTAAGTAACC
CAGGGGTGGAGGTGGGGTGGGGGATCCCAGCTCTTTCCGATCTGG
GAGATCAGAAAGATGCTCATCTGGGAGAGTGGGACCTCCCCTTTC
AGTCCGCCCACCCCCGAGGACCGCACTTGGAGGGCCGCACTTGGA
GGACCGCGCCTCCAAGGCGGGCGCGGCTGACGCCCGGGGCCCAGG
CCATGTGGGGCGTGCGTGCCGGGAGAAGTGTCCCAGCGCCTCACC
CCGCAAGCATGAGAACCACGAAACCCCCGGGGCCGGGAAACCGGG
GCGGGGCGGGGCGGGGCGGGGCCGGGAAGCGGCGCGGGGCTGGGC
CGGTGACGTCGCGGCCCCGCCCCCGGCCGGCTCCCTCCTCCCGGA
GCGCTCCCCAGCGGAGTCCTCACCTGCAGGCACAGGTGTGCGCAC
TTCGCATCTCGAAGGCCGGCCAGCAAGCTTCAGGTGAGCGCCAGC
AGCA

In some embodiments, the intronic domain that promotes trans-splicing primarily in the brain comprises or consists of sequences from the gene CSNK1E. In some embodiments, the sequences from gene CSNK1E comprise or consist of:

(SEQ ID NO: 486)
GAGACCCCATCTCTACAAAAGATGGAAAAATTAGAAAGGTGTGGT
GCTCGCCTGTAGTCCCAGCTACTCAGGAGACTGAGGTGGGCAGAT
GGCTTGAGCCCAGGAGTTCGAGGCTGCAGTGAGCCGTGATCACAC
CACTGCACTCCAGCCTGGGCAACAGAGCAACACCCTCCCTGCTTC
AAAAAAAAAGTAAATAAAAAGATCTCCACAATGAACAGGTATTCT
GCATGACTTCCTTGGAAGAGATTTCCAGAAATGGAATTGCTGCCC
TAAGGCAAACGCTAATTTACGACTCTTGGGCAGCTGGAACCTGTC
CGACAGAGCGGAGACCCTTATCTCTGCCACCACCAGCAGGCTGAT
ATTTTCAAGAAAAAAATTTGCCAAATACAAGCAGTCTCAGATAAT
ACAAAGAAGATGGAAAGTTTCCGGAGCGCAGGTGTCAGATCCAAA
GATAACTTGACCCAAACACCCCGATCTCCATGTCCCTATCTCTCT
CTTA

In some embodiments, the intronic domain that promotes trans-splicing primarily in the brain comprises or consists of sequences from the gene SUN2. In some embodiments, the sequences from gene SUN2 comprise or consist of:

(SEQ ID NO: 487)
GCTACTCGGATGTGGACCAGCAGAGTTCCAGCTCGCGGCTCCGAA
GCGCCGTCTCACGGGCGGGCTCCTTACTCTGGATGGTGGCCACTT
CGCCAGGTGGGCTGCAACCCGTGCCCAGTTACTTCCATTCTTGGT
GATAGTGCTTTCTTACCCATCTCTCTGCAGTTGGGCATCCTGCCC
ATGCAGAGAGACTGTAGGACCATTGGGGAAAGGTCATTCTAGAAT
AATCACTGGGAGCCCAGAGCTGTGGGGACCCCCTTGCGTGGAAGG
GAAGGAGTAGCTGCAGAGCAGGACAGAGGGTGTGGGCAGGTGGGA
TCCAAGGACCTGGGTCCCGGTAGGGCAGGACAGAGGCATACTGGC
AGGTGGGGGTGGGGTAGGGGCAGGGAGGACGGCAGACGGGACAAA
GGAGCGGTGCCCCCAAACACCATATGGAGCCTTCGGCTTGATGGT
CATTCTTCCAGGCCGGCTCTTCAGACTTCTCTACTGGTGGGCTGG
CACC

In some embodiments, the intronic domain that promotes trans-splicing primarily in the brain comprises or consists of sequences from the gene SUN2. In some embodiments, the sequences from gene SUN2 comprise or consist of:

(SEQ ID NO: 488)
CTCCCTCCTGAGCAGATCCGAGGCTGCTGGGAGGCCTCAGGAAGG
CTGGGCAGGGAGCCCCGGGGAGCTGGAGAGAGGGCAGCATGTGGC
AGGCACAGCGCCCCTCCCATGCCCGGCCCTGGGTGCGCCCCAGCC
TGGCCTGCGGTGGCCCCTGGTGCCCTGGGCTGTGACTGCCCTTCA
CTTGTCTGTCCTCCCCACCCCACGAGTCTATGGCAACCTAAGGCC
AACATCCTTGCATTCAGGCCCATTCCAGCACTTTCCTGTGGGGCT
CACACATGGTGGGTGCTCAGTCATTCTGAGTGGTGTGCATGTCAG
ACCCCAGCACCTAGTTCTGAACAGCAGCTTGAGGGGTGGCTGGTG
AGGAGAGGGATGCAAACAGGAAGCTGTCAGAAGAAACAGAATGTC
AAAGAGCTCCCATTTCATTCCTTACTGCACACCGGGCCTGTGTGG
GGCTGTGGGGATGTGAAGGAGGGTGGGTGACTTGTGGTCTTGGCT
CACA

In some embodiments, the intronic domain that promotes trans-splicing primarily in the brain comprises or consists of sequences from the gene ATF4. In some embodiments, the sequences from gene ATF4 comprise or consist of:

(SEQ ID NO: 489)
GGACCCCGGGATCCCTCTCCCAGCAGCCGCCCATCTAGGGCCCTG
AGCCAATAAGAGCTCGAGATATATATGATTCACCGGGCCTGGGCC
AATCACCTTGACGCGCGCTCTTCGCCAGCACCTCGCGCGATAACC
TGGCGTCCTCGGCCTTCACAATAAAAACTCTTCGCCGGAAAACGA
CCTTTCCCCGCCCACTGCGCTGACACCGGAAGCGAGGCGTGTCTG
GGAGATCACTCCGCGCTCCGGCGGCGAAGGAAAGAACGGACTCTG
ATCATAGAAGCCTAGTAAAGTAGTACACCTCTCTCCTTTCGTGAG
GCCATAAGAACAAACTCCTTTTCTCGTCACAGCTACGCCCTGGGC
ATAAACGGTTGGGGCGTCAAAGGGAGGGAGGGAAGGGAGCGGGCG
GGAGGAGACGGTCACGTGGTCGCGGCGGAAGGATGCGTCTGTGCT
GCGTCCCCATAGAGACGAAGTCTATAAAGGGCCGGCGGGCGGCCA
CGGC

In some embodiments, the intronic domain that promotes trans-splicing primarily in the brain comprises or consists of sequences from the gene ATF4. In some embodiments, the sequences from gene ATF4 comprise or consist of:

(SEQ ID NO: 490)
TTCTACTTTGCCCGCCCACAGATGTAGTTTTCTCTGCGCGTGTGC
GTTTTCCCTCCTCCCCGCCCTCAGGGTCCACGGCCACCATGGCGT
ATTAGGGGCAGCAGTGCCTGCGGCAGCATTGGCCTTTGCAGCGGC
GGCAGCAGCACCAGGCTCTGCAGCGGCAACCCCCAGCGGCTTAAG
CCATGGCGTGAGTACCGGGGCGGGTCGTCCAGCTGTGCTCCTGGG
GCCGGCGCGGGTTTTGGATTGGTGGGGTGCGGCCTGGGGCCAGGG
CGGTGCCGCCAAGGGGGAAGCGATTTAACGAGCGCCCGGGACGCG
TGGTCTTTGCTTGGGTGTCCCCGAGACGCTCGCGTGCCTGGGATC
GGGAAAGCGTAGTCGGGTGCCCGGACTGCTTCCCCAGGAGCCCTA
CAGCCCTCGGACCCCGAGCCCCGCAAGGGTCCCAGGGGTCTTGGC
TGTTGCCCCACGAAACGTGGCAGGAACCAAGATGGCGGCGGCAGG
GCGG

In some embodiments, the intronic domain that promotes trans-splicing primarily in the brain comprises or consists of sequences from the gene KIAA0930. In some embodiments, the sequences from gene KIAA0930 comprise or consist of:

(SEQ ID NO: 491)
GTGGGGGTCGGGAGTGGTGAGGGAAGGGTGGCGCATCTCAGAGCC
CTCGCAAATTTCTGGCCAGAAATGTGGGGGTATGGGCGCAGCTTG
GCCAAGGCCATGCTGCAAGGGCCAGGGTGGCCGTCCTTCCTCACT
CCTGTCTGGCCTCCCTCTGCAGGGTGCTTCAAGGATGACCGCATC
GTCTTCTGGACTTGGATGTTCTCCACCTACTTCATGGAGAAATGG
GCTCCCCGGCAGGACGACATGCTTTTCTATGTGCGCCGGAAGCTG
GCGTACTCCGGCAGCGAAAGCGGTGCAGACGGGAGGAAGGTGAGT
GACCCCCGGCCCCTGCCCCTGCGTCTGTGCCCCAAGCATCCCTGT
GGTGTCTGCTCTCGGTGTACATGTGGGCTTTCCTACAGTGCTCAT
GCATATCAAAGAGGGTGAGTGATATTTATTGAGCACCTACTGTAT
TCCAGGAACTGTGTAGACAGTCTCATGTGCAAATCTGGATGTGCT
GGGT

In some embodiments, the intronic domain that promotes trans-splicing primarily in the brain comprises or consists of sequences from the gene KIAA0930. In some embodiments, the sequences from gene KIAA0930 comprise or consist of:

(SEQ ID NO: 492)
AGAGCTTGGCTGCATATTTTGAAGTGTTGAATCTGTCTAGAGAGG
GGACAGACCCACCTATGTTGATCCTGCTGTGTGCCCGTGGTCTCG
GTTTGTCTGCATGTGCCCCGAGGAACCTGTGGCTCAGAGCCCCCG
CCCATTGTCCCATCACCAGCGGCAGCGCTGGAATGGAAACCCAGA
TCTTCCCTACCAAAGCCTGTGGGCTTTTTCTCTTTTGCAGGAAAA
TATTACAAATATTTATCATAAAGAAATACCATTTGTAATAGAAGG
AGAAGTCCCTGTGAGGATCAGATGAGCACGAAGCTGCGCAGACGT
GGGGCAGGGGTGGGGCGGGTGCTGTCTCCCTGCGGTTCCCGCTTG
CAGCCTTGGCTGGCTGGAGGGCGGCTGTCCCTTTAAGTCGGGTTC
CCGCTGGGACTGCCGCAGCCAGGGGGAGTGGGTGGGGGTGAGAGA
TGTTGGTGAGAGCCCTGCCTCTCCAAAGGCTCCCCCCCAGCCAAG
CCTT

Intronic domains that promote trans-splicing primarily in liver tissue

In some embodiments of the compositions of the disclosure, there may be an intronic domain that promotes trans-splicing primarily in liver tissue.

In some embodiments of the compositions of the disclosure, the intronic domain is derived or isolated from human genes that feature exons that are primarily included in liver tissue during the process of RNA splicing.

In some embodiments, the intronic domain that promotes trans-splicing primarily in the liver comprises or consists of sequences from the gene MASP2. In some embodiments, the sequences from gene MASP2 comprise or consist of:

(SEQ ID NO: 493)
GTGAGTTAAAACAAAGTACACCACGATCAATGACGATTGCTTTGA
AATGTAAACATATTTCTATCTGCAGATATGTGAAAGGAAAGTGAG
AAAATCGTGTGTCAGCATGATTTCTTTTGTCTATCTTTAAATGAT
AAAAAGCCAAGAAATAAGTTTTCAGAGGACAAAAAGGATGGCTAT
GGAAAATGTGATTCTGAATCCCGCCCTTTTTTCTTTCTTTCTTTA
TACTTAACTTTAAATTTACATAAGCGAGCCCTTTGTTTTAAAGCT
AGTTTGGAAAGCTGGTGATTAAAATGGGCAACTTGGCCAGGCGCG
GTGGCTCACACCTGTATTCCCAGCACTTTGGGAGGCAGAGGCAGG
CAGATCACTTGAGGCCAGGAGTTCAAGACCAGCCTGGCCAACGTG
GTGAAACCCCGTCTCCACTAAAAATACAAAAATTAGATGAGGTAG
TAGCCCCGTCTCCACTAAAAATACAAAAATTATTAGGTGGCGGGC
GCCT

In some embodiments, the intronic domain that promotes trans-splicing primarily in the liver comprises or consists of sequences from the gene MASP2. In some embodiments, the sequences from gene MASP2 comprise or consist of:

(SEQ ID NO: 494)
TTCACAACAGCCCTGGGGCAACTATTATCGTCAACCGCCCCCCAT
CTTGCAGATGAAGAAACTGAGACTCAGAGAGGTTACACATAACCA
TGGGGAAATGGAGAGCTAGGATTTAAACACAGCTCCAGTCCCATG
CAAGTCCCAAAGCCTCCCCTGAGCTCTTTTCCTCGGAGTTACACT
CTCTCTTGGTTTTGAGCCATGGCTCTGAAGTTCCTGGCATGGAGC
AGATGTTCTATTTGTGGGGACTGCACCATCTTCCCACATAACCCA
GCTTTCTCACATACCTTTTGCTCAGTCCCAGGGAAAACAAGAAAC
CTTAATTTGCAAAAGCACCTGAGAGTTTAATCTTGCCTTTGGTTT
TAAAGAACCCTACCCCACGTATGGTGAGCTCTCTTTACATGTTAA
GTATTCTTTATAAACTAAGAACTTCAGATGGCTCGATTCAGTCTT
GGCTGCTGTAGACCACATGAAAACCAACAAGCCTGGTTTCCTCTG
CGCA

In some embodiments, the intronic domain that promotes trans-splicing primarily in the liver comprises or consists of sequences from the gene AGMAT. In some embodiments, the sequences from gene AGMAT comprise or consist of:

(SEQ ID NO: 495)
GTAAGGAGATTCCACCAGTGAAGATCCTGGGGGTGGAGACACTTA
ACACCCATTTTCCTGGGACTTGAAGCGAATCTTTAGGAATCAGGA
TAGGATTTTTGTTTGTTTGTTTGAGACGGAGTCTCACTCTGTCGC
CCAGGCTGGAGTGCAGTGGTGCGATCTCGGCTCACTGCAACCTCC
GCCTCCCAGGTTCAAGCGATTCTCCTGCCTCAGCCTCCTGAGTAG
CTGAGATTACAGGCACATGCCACTACGCCCGGCTAATTTTTGTAT
TTTTAGTAGAGATGGGGTTTCACCATGTTGGTCAGGCTGGTCTTG
AACTCCTGATCTCGTGATCCACTCACCTCGGCTTCCCAAAGTGCT
GGGATTACGGGTGTCAGCCACCGCGCCTGGCTGCCAAGGTAGGAT
TTTAAGAGCAGAGCACCTAGGCCTTTTCTGTTCTCTGGGCTGAGG
ATAAGTTTGAATACCCTCTTACTGAAGGGTGATTGATTTTGGCAA
GGGC

In some embodiments, the intronic domain that promotes trans-splicing primarily in the liver comprises or consists of sequences from the gene AGMAT. In some embodiments, the sequences from gene AGMAT comprise or consist of:

(SEQ ID NO: 496)
TGAGATCGCGCCACTGCACTCTAGCCTAGGTGACAAAGTGAGACT
CCATCTCAAAAGAAAAAAAAAAGAGGGGAGGAAGTCCGGACCCAA
GATGAGTTGCCACCTTCTTAGTTCTGGTCCTCGTACCCTCTAGGC
AGAATGGCTCTCAGATAGACGTGTATGACAAGACCCCAATGGAAC
AGACCCAAATCCCTTGCTTTTAGAGCGTGCCAAGGCCAGTCTTCT
GGACAGAGCAATTCAAAAGGTGCTAGCGTGAGCTGCTGTTTCCCA
CTCTTGCTTTAGCAAAGCTTCTCTCTGATTCCAACTCCATCAGTC
AGCTTTGACCCTGCTTCCTTACCACTCATTGTCCCTCCCTTCCTC
ACCTGTAAAATAGGATAGTTCCCTCTTCATGGAGATGGAGGGTCA
AGTGTCATGTGCACACAGAGCTGAGCATGGGGCTGGCAAACAGGA
AATGCCCTGTAAATGCTGCCAGACAGAAACCTCAGTCCCTGTCTC
TGCA

In some embodiments, the intronic domain that promotes trans-splicing primarily in the liver comprises or consists of sequences from the gene MFSD2A. In some embodiments, the sequences from gene MFSD2A comprise or consist of:

(SEQ ID NO: 497)
GCTTCCGCAATGAATTCCAGAATCTACTCCTGGCCATCATGGTGA
GTGGGACCTGAGCAGGGGCGGGCAGCCTGGGCTGAGGTGACATAG
GCTGTGGAATGGTTCTTGGAATAGGCAGAGGATGTTTCTCAGGCT
GGCCCAAGGTCATGAAAGGATGAGGGAGGCTTCTCAGGGTATTTG
GTTGGACCTTGCGAAGGTAGGACTGTGAAAGAAAGAGTTTGAGGT
TAGATGTTTGAGTAGTGAGCACAGCAAAAGCAAGGGCGAGGACAC
CAGGAACTTGGCTCAGAGGTTTGAGAGGGCAGCAGACAAGGTGGG
CCTGGACCAGCTCTGAGCTCCCCTGGGGAGCCGTCAAAGGTGCCT
GTCATCTTCGTTGCTGCCCACATGATGTCATCTGGCTGCTCTTGG
GCAGGGCTGGGAGCCACTTTGGGGTTCTGGGAAGGGCAGGAGGGG
CTCTGTGGCTCTAAAGCACCTCCCTTTAACCCCCTTTGTCCATCC
ACAG

In some embodiments, the intronic domain that promotes trans-splicing primarily in the liver comprises or consists of sequences from the gene MFSD2A. In some embodiments, the sequences from gene MFSD2A comprise or consist of:

(SEQ ID NO: 498)
TGAGTGGGGTTGAAGAGCAGAGCCTGGGTTGAGTTGGGATGTCTG
GTGGGAACCTCCCAGCTGATTCATCTTCCTGCACCCCCTTCCCTA
GTCAGCAGTGCCATTTCTCATCTTGGTGGCCCTCATGGAGAGTAA
CCTCATCATTACATATGCGGTAGCTGTGGCAGCTGGCATCAGTGT
GGCAGCTGCCTTCTTACTACCCTGGTAGGTATATACAGGCCCCCC
TCCTCGGGTATCTCCAGCCCCTAGTCCCCAGTTTTGAAGCTCCTT
AGGGAGAGTTCTATGCAGTGTTCTCCCACAGGCCATTCTGTGGGT
CCAGGTTAGGAGTGGGGGAGGTCTGTCCTGTACAGTTGTACAGGT
AGTGAGCTTTGCAAGAACACCTAGTCAGAGTGAAAAATGGGGGCT
GTAATCTGGCCTGGGCTCCACTTGCCACGCTGAGCACCTCCAGGC
AGGGTTACTTCCTCTTAGAGCAAGAGGCCTTTTCTTATTCATGTG
AAGG

In some embodiments, the intronic domain that promotes trans-splicing primarily in the liver comprises or consists of sequences from the gene EBNA1BP2. In some embodiments, the sequences from gene EBNA1BP2 comprise or consist of:

(SEQ ID NO: 499)
GAAGCGGAAGTCCCAATCAAAAGTTGAGCAGTAGCTGTGAGTTGC
TGGCGCTGGAGCGATTCGGTGGCACGTGGAGCCACGGCGTGGGAG
TAGGGGGCTGAAGGCAGGCAGCAGCGGCCAGGGCCGCCCTCTGCT
AGCCGCTTGGGTCTCGGGATACCCCGTTTCTTCCTGTAGGTGTGG
GACGTGCGTGCGGCGAGATGGACACTCCCCCGCTCTCGGATTCGG
AGTCGGAATCCGATGAATCCCTTGTCACAGACAGAGAGGTAGGTG
TCAGCCGAGCTCGTGGGGCTGGGCGTGGGAGAGGGGGCCTTACAC
CCCTGGGAGTGACCGTGATTGTGTCCTGCAGTTGCAGGATGCGTT
TTCCCGAGGGCTTCTGAAGCCAGGCCTCAATGTCGTGCTAGAGGG
GCCGAAGAAGGCCGTGAACGACGTGGTGAGCGTGGGCACAGGGTC
GGGGGTACTCGGACATGGGATGCGCTTCAGGCGCTGTTGGGCTTG
GGAG

In some embodiments, the intronic domain that promotes trans-splicing primarily in the liver comprises or consists of sequences from the gene EBNA1BP2. In some embodiments, the sequences from gene EBNA1BP2 comprise or consist of:

(SEQ ID NO: 500)
AGTTTCATAATACAAGTCGCCATTTATTTCCTCTACATTTATAAT
ACTGCATATATATTCAAATTGGCCAAAATCTTTCTTTCCAATCAT
CATGCTGGGATTATGGGGTTATATGTGGCACATACCATTCCTGTA
TATGTCAGGATGGAAAAAGTTTTACCTGGAATGAATTTTTGCCAT
TTCTGATCCACATTGTACTTCACACAGTGATTTCCTGAAGGAAAT
ATAATGATCTGTTCATCGAAGTAGAAGATATTGTTGGCCACGTGG
GATCGAAGACCAAAAACATGCAGCGTCTGAGCTACCACGGCTGAC
ATGATCTCCCGCCAAACAGTTCTGCTGAAGACCAAGGCAGCGCTT
CAGAGTGGAGAGCACCGCCCCGCTCCGACCCCGCCCCCGGCCCGG
ACTCGCCCTCCCCGCCCTTTGCTCCTCCCCTTGTCTCTCCCCTTT
CTCCTCCCCTTGTCCCTCCCCTTTCCCCTCCCCTTGTCCCTCCCC
TTTC

In some embodiments, the intronic domain that promotes trans-splicing primarily in the liver comprises or consists of sequences from the gene PRDX1. In some embodiments, the sequences from gene PRDX1 comprise or consist of:

(SEQ ID NO: 501)
GTGAGTGAGACTCCGTCTCAAAAAAATGTGGAATAGAACAAACTT
TAGTTTTAGGAACTAGACTTTATATTGTAGATCTAGTTGCAATTG
ACACGTCCTTGTTT
TATGGGAGGCTACATACTTTAAAGGTTTCCAGGGGAGCAAATAAT
TAATTATGATTTTGTGTATGTGTGTTTGTGGGGGGTAGTCATCAG
GAAATGTCTGAGGCCTTTTGCCTTACCGTGTGGGTCTGGCTAATG
TGTAATGTTTTGACTCAAGATTGTAAAGTCAAATTATTCATCCGG
GGAGAGGATGGTCGTGTAACTGAATCAGCCTCCCAAGATGAACTT
TCCCTCTGGCGTAAGTTTAGGAGTCCTGATATTTTATTTTCCTTT
ACAACAGAAAGCAGCAACCCTGTAATTAAAGAGAACAATGAAGGA
AGCTATCTAAGCCATGAGGGTTACTGAGGATCTGCGCTGGGGTTA
AGGCTGCGAACTTAAGAAATATTTGGATAGACAGT

In some embodiments, the intronic domain that promotes trans-splicing primarily in the liver comprises or consists of sequences from the gene PRDX1. In some embodiments, the sequences from gene PRDX1 comprise or consist of:

(SEQ ID NO: 502)
GCCTCCGGGTTCAAGCGATTCCCCTGCCTCAGCCTCCCAAGTAGC
TAGGACTACAGGCGCGCCACCACACTCGGCTAAGTTTTTGTATTT
TAGCAGAGACAGGGTTTCACCATGTTGGCCAGGATGGTCTCGATC
TCCTGACCTTATGATCCGCCCTCCTAGGTGGCCTAAATCTACATT
TTTACATAAGATAAGGTGAATGTTTGAAGAAAAAGGGAATGGAGG
AAGAAAACAATTATGTGGTGGACTCAGGGTAATCTAGTAGTAAGG
TACTACTAGAAAGGTAGAAAGTAGTCTAGTAGAAAGGTAATCTAG
TAGTGAACATTTATGGAATTTGTGGAATAGAGGCCAGGTGCGGTG
GCTCGCGCTTACATTCCTAGCACTTTGGGAGGCCAAGGCGGGCGG
ATCACCTGAGGTCAGGCGTTGGAGACCAGCCTAGCCAACATGATG
AAACCTCGTCTCTACTAAAAATACAAAAAATTAGCTGGGCGTGGT
GGTG

In some embodiments, the intronic domain that promotes trans-splicing primarily in the liver comprises or consists of sequences from the gene FGGY. In some embodiments, the sequences from gene FGGY comprise or consist of:

(SEQ ID NO: 503)
CTGCCCAAGACCATGGGAACCCACCTCTTGCATCAGTGTGACTTG
GATATGAGACCTGGAGTCAAAGGAGGTCATTTTGGAGCTTTAAAA
TTTGATTGCCTTGCTGGATTTTGGACTTGCCTGGGCCCTGAAACT
CCTTTGTTTTGGCCAATTTCTCCTGGAATGGCTGTATGTACCTAA
TACCTGTACCCGCATTGTATCTAGGAAGTAACTGACTTGTTTTTT
ATTTTACAGGCTCATGGGTGGAAGGGACTTGACTTGTCTCAGATG
AGACTTTGGACTGTGGACTTTTGGATTAATTCTGAAATGAGTAAA
GACTTTGGGGGACTTTTGGGAGGGCATGATTGGTTTTGAAATGTG
AGGACCTGAGATTGGGAGGGGCCAGGGGCGGAATGATAATGATTT
GGCTGTGTCTCTATTCAAATCTCAACTAACTTATATCTCCCAGAA
TTCCCACATGTTGTGGGAGGGACTTAGGGGGAGGGAATTGAATCA
TAAG

In some embodiments, the intronic domain that promotes trans-splicing primarily in the liver comprises or consists of sequences from the gene FGGY. In some embodiments, the sequences from gene FGGY comprise or consist of:

(SEQ ID NO: 504)
TAAGTCAAATTAAACATCCTATTCTTCCCAGTCTCAGGTTTGTCT
TTATCAGCAGCATAAAAATGGACTAATGCAGTAAGTTAGTAACTT
TCCCCCATATTTTTGTTAGTGGAAAGATAGAATAGTTTTTTTTTG
TTTTAATGGAAAGATAGAATAAGAATCTTGGATTCTTTAAGGCCT
GAACTTAGATGCCAGCAGTGGCTGGGAAGGGCACAGGAAGGTGAT
AGTCCATGATGACGGGTTGATACAGACTTACTTAATATATGTTGA
CTATGTTTTATCCTTATTATGCCTTCTTATGCCTTCTTATGCCTC
TTTCTTTACCCTTCACCTTACAGCCAATCCAAGAGTTGGATATCT
CAAAAAAATTAGGAGGCCTGGGTGCAAATGTTTCCTTTCATTTCT
ACCTTTCCTATACCCCTATTAGGATGTGGAGGTGAACAGATGCCT
GAAAATCTTGAAAAGGTTGTCCATGGGTCCAACTCTATATTAACT
AAAT

In some embodiments, the intronic domain that promotes trans-splicing primarily in the liver comprises or consists of sequences from the gene LEPR. In some embodiments, the sequences from gene LEPR comprise or consist of:

(SEQ ID NO: 505)
TCTTTCTCCTTCCCCTTCTCTTCCCTCCCCTCCCATTATATAATC
TATGCTGTTATGTTACCTATTTTATCCACTACCCCATCTTTACTG
GAGGTGTTGTTCTAGTGGACTCTCCAGGAACTCCTCTATCCTCCT
ACTTCAATCAGGACTAGGCTTTCTCTAAAATTACTATAAGCCATC
ACTCTAAACTTTCCCTTAACTGGTTTCTGGTTAGTACTGTTTCCT
GGATTCCTTTCCTTCATCTTTCTTAACTACACCCTTAAGTAACCT
CCTAAGAAGTATGCGATAAGTCTCCATCTCTCCATGCCTGAACTG
CCTTTTATTCTGCCCTCCTCGATTGTTAGTTTGGTGGAGTATCAA
TTCTTAGAAATAGCCTTTCCAAGAGAACATTGAGATAATTCCTGC
CTTAACATATAGTATCCAGTGTTGCTTCTGAGCAGTCAGATACTA
TTGTGATTTTTACTCCTCACTGTTTACTTATAATTTCCTTTTTAA
ACAG

In some embodiments, the intronic domain that promotes trans-splicing primarily in the liver comprises or consists of sequences from the gene LEPR. In some embodiments, the sequences from gene LEPR comprise or consist of:

(SEQ ID NO: 506)
AAAAAAAAAGAAAGAAAAAAAAATTCTCCTCATTTTTGTTGTTTTGAATTT
TCAGTGATGAATCTGGAGTGGTTCTTTTTTTATTTAATGTGGTTATTTTTT
GAAGAGACTTTCAATTCCATGTCTCCTGTCAATTCTAAGAAATTATATTAT
TTCTTTGATAATTTCCTCTCCTCTTGTTTCTTTGTTTCTCTTTCTAGAATC
TGTATGTTGGATGATGGACCCTCTGCGTAGATCTTCCACATCTCTTACCTT
TTATCTCATATTTTCGGTTTCTTTGTTTTGTGTGTGTGTGTGTGTGTGTTA
TATTCTGAGACCTTGCCATACCCTCTTCTTCCAGTGCTTCTGTTGGATTTT
TATATACTATTATTTTTGTAAATTTCCAAGTATTCTTTCTCAGAGTATTCT
GTTCTGGTTTTATGCATATTTTCTTAAATGTTGCTAGAAATACCAATTCGA
GGATTTTTGTTCTTTTATTTTAAAAATTCTTTTTTCTTCT

In some embodiments, the intronic domain that promotes trans-splicing primarily in the liver comprises or consists of sequences from the gene GBP7. In some embodiments, the sequences from gene GBP7 comprise or consist of:

(SEQ ID NO: 507)
AAAGAAAAACTATGAGGTTCTTGCTTCAATTTATCATGGGCTTGAAAGAAC
AGTTTGACAGGGCACTGAGCTGGCAAGGGTTTGAGGATATTGTCAGTGTCT
CATATTTGCTAGTGAGAGTGTGAACTGTTGGAATCATATATGAAATATTTA
AGTGCACATACCTTTGATCTAGCATTTCCACATTTAGAAATCTATCATATA
GATTGATTTACTCAAATTGAAAATGGTATATATAAAATAGTACTTATGTTG
CAGCATTGTTGTAATGCCAAAAGACTGAAAAAAACATTAAAGCCCCTCTTT
CAGGTACTGATAAGGGAAATCTTGGGAAACACTCATAAAGGAATACTCTAC
AGCCACTAAAATGAAGGGAGAAGCTCTATTTGAACTAACAATGACAAGTCT
CCAAGATGTATCATAAGTAAAAAAAAAAAATGATGCACGAAGCATGTATAA
TTACTACCACTGAATTTAGAAAATAATAAATGTATGCATA

In some embodiments, the intronic domain that promotes trans-splicing primarily in the liver comprises or consists of sequences from the gene GBP7. In some embodiments, the sequences from gene GBP7 comprise or consist of:

(SEQ ID NO: 508)
GAAAGTATCAGATTTGGCTTGATATGAAATGATACTGAATGATCTCAAATT
GCTCCATTTGTTTCTGTATCTGCCTGACTCATCCTATACAACTAGAGTAAA
ACATATTCCTTGTCCCAAACTGGAGCTTGACAACCAGTGTTTTATACAGGG
TCAGCAGTCCCCATTATTTGCTAGATGAGCATGGCCTTGCTATATCGGTGG
AAAGGGGAATCAATTAGAATTAAATCTGCATTCCCTCTCAAAGGGCATTCA
TTGTGCTGGAACAAAATTCCTCCCCACCTCCAGTGAGATGGTGCTGAGGAA
ATGTCACAAAGGTGTATATCATGGATACTCCCCTTATTTAGGTCCAGTCAT
CTGGAAATGTCAGACCAACTCATCAAAGATTTTCTAAGATGTTAGAGAAAT
GGACACATGTTTAACTAATGAAATTGTTAAATACAAACCTCGGTCTCTTAA
AAATTGGGGATTTTCAAAAGTAGGCTCTTTTACTTTTATA

In some embodiments, the intronic domain that promotes trans-splicing primarily in the liver comprises or consists of sequences from the gene ENSA. In some embodiments, the sequences from gene ENSA comprise or consist of:

(SEQ ID NO: 509)
GTGGGTGAAACATCCCTGTGGAGGATCCCAGTTATTGAGTCTTAGTCACAG
ATGATAATGTGATGCTGATTTGCATTTACCTTGCTGGAGGCAAGGCAGGCG
TGGGCAGGCTGTCACAGTCATGGAACATGTGTGGGAGCTGTGAGAGTCAGA
AGGAAAATACATTATCCTCATGAACAGGCCCAGTTTCTATTCCTGGTCTGG
GAGAATAATCATTTTGAGGTTGTTGGCTTGTGGTTGATCTCCTGGTGGCTA
GGATAGCCCCATATTTATCCTTCTCTATATCTTATTCATTTCTGTTTCTCC
AGTTCCAGGCTATTGCCCTGTACCCAGTAAGGTCCTCAGTTACCCTTTATG
AATAAGTGATTTTGGTGGAACGATCCTCTACTGAGTGGAGTACAAAGGATG
GCAGGGGCTATCCCTTGACTCCAAAACTATATACTCTAGCTGGGAAATTGC
TTATGTACCTGAAAAAGTCAGAACACACTGGGGAGAAGAC

In some embodiments, the intronic domain that promotes trans-splicing primarily in the liver comprises or consists of sequences from the gene ENSA. In some embodiments, the sequences from gene ENSA comprise or consist of:

(SEQ ID NO: 510)
GAAGTGAGCACCAGCCATTAATTATACTCCTGCAGCAAATGTATTTTTCTT
GTTCAGAGTGGTCCTCCTTACCAAGAGAGAGTCTACTTTCATAGCCATGTA
GCTCCTGATCAGATTTTTAGTTCCCAAAGGTACCTAAAGCTGACTGAGGGA
TTATATTATCGTTTTTTAAAAAAAAAATTTGGCCAGGCGCAGTGGCTCACG
CCTGTAATCCCAGCACTTTGGGAGGCCGAGGCGGGCGGAGCACGAGGTCAG
GAGATCGAGACCATAGTGAAACCCCGTCTCTATTAATAATACAAACACTTA
GCCGGGTGTGGTGGCGGGCGCCTGTAGTCCCAGCTACTCGGGAGGCTGAAG
CAGGAGAATGGCGTGAACCCGGGAGGCGGAGCTTGCAGTGAGCCGAGATCG
TGCCACTGCACTCCAGCCTGGGCAACATAGCGAGGCTCCATCTCAAAAAAA
AATTTTTTTATTTTCCTTACTCTCCTCTGCAATGACTGTA

In some embodiments, the intronic domain that promotes trans-splicing primarily in the liver comprises or consists of sequences from the gene SLC39A1. In some embodiments, the sequences from gene SLC39A1 comprise or consist of:

(SEQ ID NO: 511)
GTGGCCCACGAAGCTACCCAAGACATCTCTGCACAGCCCTAGCCTTTTTGG
CTTCACCCACTCCGTTCGGGAGTTGGGGACCCGGCCTCTACATTCCTTAGG
GGAACTCCAGCTCCAGGTCCTATTTGGAGGCGGCTCCTAGTTTCCTAGTTT
TCTGGGGGCACCAGGTGAAAGGGTGATGAGATATCCATACAGCCTTTCTCA
TGCCTCGGTTTCCCCCGTGCAGTCTCCCTGAATAAGTCACTTGGGAAAGAA
AGTGTGAGAAGAGAGGAATGTTCCTGGTACAGGAGTAGATGAGTCACCCTG
GGTAGGGGAAGGTCACAAAGTGGCTGGGGCCAGGGAGCTGCACTCTGCTGA
CCAAGCTTCCCCATTCCTTGCCCCATCTTCCTTTGCATGATTTTTCCCAAC
CACTCTGGTCTGTCTTCCTAGGTCTGAGAGTCACTGGAGCTACCAGAAGCA
TCATGGGGCCCTGGGGAGAGCCAGAGCTCCTGGTGTGGCG

In some embodiments, the intronic domain that promotes trans-splicing primarily in the liver comprises or consists of sequences from the gene SLC39A1. In some embodiments, the sequences from gene SLC39A1 comprise or consist of:

(SEQ ID NO: 512)
AGACTTGACAATGCGGGGTCTCCCTCTTTTAAAAATATCCATATAATCAAA
GCCTTTGTTGACTGGCCCGCTCCGGCCCCTCAGTCCTCCACAGATGCGCCC
CAGGGCCCCAGCCCCGAGGTCCTTTTCCTAATATCCGCTCTTGCTCTCGCT
CACTCTCCTCAGGTCGCCTGCTCCGCCCGCTCCCGCTCCCGATCTCTGATT
GCTCCTAACTGACGTCACTCCCGGTCTGTCCCCGCCCACTCGGTGCTGCCA
TTGGCAGTCGGTCGTGGGTACGCGGCTCCTGTGGGGGAGGGGGGAGCTATG
GGAGGGGGCGGGAATTCCCGACTCTAGGCCGGAAGCGCGCGGAGACCATGT
AGTGAGACCCTCGCGAGGTGAGTGCGGGCTGTCTGGCAGCGGGGTCCATGT
CGGCTGCCCAGGCCTCCCCGGGCCCGGGAACAGGAGTCCCAGCTCACGCAT
TCTTCATCTGCTTCCCGCGACGCTTGGCGGCCTCGCACCC

In some embodiments, the intronic domain that promotes trans-splicing primarily in the liver comprises or consists of sequences from the gene RAB13. In some embodiments, the sequences from gene RAB13 comprise or consist of:

(SEQ ID NO: 513)
GTAAGGCTCCTAAATTCTAGTCCCTCAACCCCTGGCTCTGACCCTTCCCCC
GTGCATGTCTCTGTATAGTAACCAAACCTCTCACCTCTCTGAGGCCTTCAG
GTAACTCTTCAACTGCTGCCAGCTCCTCATTTTCTTGGTACCATCGCCTTA
GTCTCCCCAAATCCACAAGAACTCTTCTCTATTTTTCTCTGTTCCCCTCTT
CCCCTACCCCCACCACCTAGAACTCTCTCGATCCTATCTCCATGTTTCCTG
TATCCCTGTCCGCCACATCTTTTTCTCCCGATCCGATGCTAGCCTCTTTCT
GTCTTGCTGAGCTTCAAACTCTCCCACTGTGTCCCCACCTCACCATCTACT
TCCCAGGTTCCCTGCCCCAGTCTCCCTGCACCTCAGCTGAACAAGGGTTTG
GGAAGGGTGGAACAAGGTGGCTGGGTGTGCTGGAGGGTGGCCCAAGGACTG
TGATCATGAATATGCAGCCAGCTTGGTGAGGAACGTGGAA

In some embodiments, the intronic domain that promotes trans-splicing primarily in the liver comprises or consists of sequences from the gene RAB13. In some embodiments, the sequences from gene RAB13 comprise or consist of:

(SEQ ID NO: 514)
TCGTCGTGGTTAAACCTCTACTTCCCTTCACCCCAGCCTCTCTGGCCTCAG
AATGGAAGAGTAAGGTCCTAAGAGGGGAGCTACAATTACCGAAAGGCTGTG
GAAAATAGCGAGAGAGACAGGCCGGCCTAAGCTAGGAAGGGAAAACCGAGA
GAACAGAGGCCGGGCTGGCAGCCTTGGCCTGCTCAAAAGGGTGGGCGGGCA
GGGGGTGGAGCCCGGAGGGGTGCCGCCTCTCCTCTTTCCCGGAGCCTGGGC
GGAGAGGGAGGAAAACTTCTTCCTGGCCTGGGCTCCGTGCCGCTCTGTTTG
CCAACCGTCCAGTCCCGCCTACCAGTGCCGGGCGCTCCCCACCCCTCCCCC
GGCTCCCCCGGTGTCCGCCATGGCCAAAGCCTACGACCACCTCTTCAAGTT
GCTGCTGATCGGGGACTCGGGGGTGGGCAAGACTTGTCTGATCATTCGCTT
TGCAGAGGACAACTTCAACAACACTTACATCTCCACCATC

In some embodiments, the intronic domain that promotes trans-splicing primarily in the liver comprises or consists of sequences from the gene FMO4. In some embodiments, the sequences from gene FMO4 comprise or consist of:

(SEQ ID NO: 515)
CTCCCAACGTGCTGGGGTTATAGGCATGAGCCACTATGACCAGCCTAAAAT
GGGGTCATTTTAAGTGTTTACATCTAACCAACTAATAGCTTTAACACAAAC
ATGCACACACACACACACACACACACACGTACAATGTGGAGGGTATACATA
CAATATAATATAAATTATATTATTTATACAGCAAAAGCAGATGAAGAGAGA
CAGGGCAGGCCAACTGGCACAGGTGCAGGTGTCTGGTGAGTGTGGCGCAGG
CTGACAGCTGGGCCATAGAGGCATCTCTCCTCACAATTCTGGCAGTAGAGT
ACCCTGGTTAAGTGAACTGGGCGGGGAATTTTACCTCCAGAAAATTATTTT
CTGTGCTGCATTTCCATTCTGAGCTTTGACTATCATGTGCTCTGAAGTTAG
AGCAGTGGGAGGCTAGAAATGAGTGGGTGGAGAATTTAAATAAAGGAAAAG
CCACATGTGACTTTAGTGTTGTTTGTGATTTTTCCCACAG

In some embodiments, the intronic domain that promotes trans-splicing primarily in the liver comprises or consists of sequences from the gene FMO4. In some embodiments, the sequences from gene FMO4 comprise or consist of:

(SEQ ID NO: 516)
TATGAAATGTAGCTTGCTCTAGGGCAAACTTACAGTATATTCTGTTACAAA
AAAAGGATTCAGAGTATAAAAGCCATTCCTAGAGAAGCAGTATGGCAGGTT
GGAAATAACTCTGCATTAGGAATCAGAAGGATACATTTTTAGTCCTAACTC
TGCCACCAATCAGACGTGGCCTTAGACAAGTCATCTGGGGACACCAGGTCT
TTTCAGTGGAACACACAGCCTGGCCCCCTACCAGTGGCAATCCTGTCTTCA
CTTAGTCCAGGAAATCTATTCTGACTTCACTCTCTTCCTACCTCTCAGGCT
GCTCTTTCTCTGTATTTGCTGGTATCGCCTCTTCCTCACTCCCAAAATTTA
GTGAGCTCCAAACTTAGTCCTCAGGCCTCTTCTCATTCCTATTTACACCCA
CTCCCAGGTGGCCTCATCCAGGCCTGTGGCTTTAAATATCATCATATACTC
AGGGCTCCCAAACTGTGTCTCTGACTCAAATCTTTCTCCA

In some embodiments, the intronic domain that promotes trans-splicing primarily in the liver comprises or consists of sequences from the gene CFHR3. In some embodiments, the sequences from gene CFHR3 comprise or consist of:

(SEQ ID NO: 517)
CAGATGACTATTAGAAAGATTATACCTTTCTACAGTTCTGTTTCTACATTG
TGGGCATAAGAGAAAAATATAAACATAGAATTAAATTAGCAAAATGTGAGT
ACATTTGGAGTGCTTGTAGTCACGGCTTGTCGAATGGGGGAAAGGAGGATA
AGTAACCATGACTCCAATGGAACATGTTAGCATAATCCTTTTTGATATGAA
GTCACTAGGGTGGACACAACATATTTTAGGAAAGAAGAGTTCAGGCTCTAG
AGAAAGACTGCCAGGGTTCACGTTTATCACCTCACCTGATGATCTCTGCTG
TGATGTTGGGGAAGTGCAACTGAACTTATTATATTTTTGAAATGCTAACGT
CAGTATGTAGCACAAGTTAATAACTATTAACTATTTGGATTATTTTATAAT
TTTATTTTATCCTAAACTACTCATTAGGATGCATTTTATTTGCTCATGAAA
GAGAAAATTATGATTGTTAATTGTTTTTTTCTGCTTTCAG

In some embodiments, the intronic domain that promotes trans-splicing primarily in the liver comprises or consists of sequences from the gene CFHR3. In some embodiments, the sequences from gene CFHR3 comprise or consist of:

(SEQ ID NO: 518)
AAAAAAGAAAAGAGGTTTTGCTAACCCTTTCAGAGCATTGGGAACACAGCC
AGAAGTGCATTAAATGTATATATTAACTTGGGCAATGTTGACACTTTAGGA
TGCTGAAGCCAGGTGCAGTGGCACACGCCTGTAATCCTAGCACTTTGAGAG
GCCAAGCTGGCAATCATCAGAGGTCAAAAGTTCAAGACCAGCCTGTTCAAC
ACGGTGAAACCCTGTCTCTACTAAAAATAGAAAAACTAGCTCGGCATGATG
GCGTGCACCTGTAGTCCCAGCTACTCAGGAGGCTGAGGTGGGAGAATCACT
TTAACCAGTGGGGCAGAGGTTGAACTGAGCCAAGATAGTGCCACTGCACTC
CAGCCTGGGCAATAGAGTGAGACTCTGTCTTAAAAATAAATAAATAGGATG
CTGAAAATTCCTATTTAAGGAAATAATTCTTTTTCTGATTTATTTAAGGCT
ACATTTGTATTTTCTTAATACAAATATTTTGAAAGTTTCT

In some embodiments, the intronic domain that promotes trans-splicing primarily in the liver comprises or consists of sequences from the gene C4BPB. In some embodiments, the sequences from gene C4BPB comprise or consist of:

(SEQ ID NO: 519)
TGACCTGACAGGAGGTGGAGCTCAGGCAGCAATGTGAGCAAAGCAGAGTGG
CTATAAATACAGCTGAAGCTTTGCTTGCCTGCCCGCTGCTCACTTTCTGCT
GTGTGGCCCAGTTCCTAATAGTCCGTGGACCGGTACCGGTCTGTGGCCCTG
GGGTTGGGGATCCCTGTTATAGAGCAAAGCACATAAGCACAATGCGTGAGA
ATTGGAAAAGTCTCTAAAATTCTTCTAGGCTCCAAAACTAAACAAACAACA
CTGAACAGATCCATTTTCATTGAGATTCTTCTTTTAGTTCATATTTATTAC
TGGGATAATATTAATAAGTATATTTAAGCCAGAAAATATAAATGATTTGCC
ATGTTGAAAACCCAAATCTTTACTTAAGCTAGCCTGGTTCTGAGCCCCCAT
TCAGAGAGCTAACTGCTGCTCACTGGCTTCAGAGTATATAGCTGGGTCTCA
GTCCTTATGTTGTAACTTGTGAAAAATTTTCTTTCTTCAG

In some embodiments, the intronic domain that promotes trans-splicing primarily in the liver comprises or consists of sequences from the gene C4BPB. In some embodiments, the sequences from gene C4BPB comprise or consist of:

(SEQ ID NO: 520)
TTGGTTTCAGATTTACCTCTTATTGGGCTGAAATTGCCAACAGCAGTTGTA
TAATGCACCATGTGTGTGTGTGTGTGTGTGTGTGTGTGTGTGTGTGTGTAT
GTGTGTGTGTGTGTGTGTGTGTGTTGTTCTCAGAATGTGAAAAGGCCAAAT
GTAGTGGTTGTCTAAGGCCAGAATTAGCAACATATTATTGGAGAATAGAGG
GCTTAATTTCCAGTCTGTAAGGAGAATGGAAAACTCTGATCTCAGCCGAGA
GAGTAACTCGTATTTTAACATGAGACCATGAATTATGAAACCAAAACTAAC
CGCAACTTATGAGGCAAAAATTAACCTTCCATTTTGGAAAAGATGCTTAGA
ATAGTTACATCCTTAAAAATGACCCACTGGGACAGAAGGAGAGCTAGGACC
CAAATTCTCCTCCCAGTCTTGCCCACATCTGGCTTACGGAAACAGGTTTCC
AAAGCTGCAGGAAAATTGCTTACTGCCTCTTGAATGAAAC

In some embodiments, the intronic domain that promotes trans-splicing primarily in the liver comprises or consists of sequences from the gene EPHX1. In some embodiments, the sequences from gene EPHX1 comprise or consist of:

(SEQ ID NO: 521)
AAGGCGGCAGCGGGGTTGGAAACCCACCCCCACACAGTCAACACC
TGCTGTCACACCAGGCTAATCAAGAACACTTTTCTTTGGTGCATT
AACCACCTGCTATTAAATAAAGGGCTGTTACTAGGTGGCAAAGTA
ACACACTGCAGTAGAGCCTATTTGTAATCTGAGATCAGTGGCTGT
AAGGCACCATCCTTGAGCCAAGCAAGTACTAATATTCTTGTTATC
TTAATTAGAATGCAAGAGATCGTGTTTTTAAAATTCTCTTTGAAT
CTGTCACTTTGCCCTCCTCTTCCTGGGTGATACACCTGGTAGTGC
TGGTGGGGGCCATCATAATGCCCCTTGTCCCAGATTCCCTTCTTT
TAGATGGGACTCGAGCACTGATCATTTCAGCCCTGTATCTCTCAG
GTCAGCGTGGTTCAGTTTGCTGTGCAGAGTCCAGGGGAGATAACC
ACGCTGTGCACACATGAGATTGGCTGACTTGGCAGGACTGTGCAA
TTGT

In some embodiments, the intronic domain that promotes trans-splicing primarily in the liver comprises or consists of sequences from the gene EPHX1. In some embodiments, the sequences from gene EPHX1 comprise or consist of:

(SEQ ID NO: 522)
TAAGGAGGGTGGTTTGTAAAGTTCACTGGGGAGGGTGATCCCTCT
TTATTGTTCTAATATTATGCTCGCAGCCTCCTTGACAACATCATG
CAAAATGTGTCTTACTAGCTTCTAGTGCATAAAATATTGGTGGAG
CTCTTCGCTGTGCTGGGCCAGTCACCAGTGCTGGGCACTGTGGGT
CAAGGCAGTGCCCAGGACGGGCACAGCCCTGCCTTCCCAAGCTAT
GGTTTATTGTGGTAGGATCTGCCTGCCCAGTGAAGTGTGTTCAGC
TGCTCCTGTTGCTGGGGGAAGCTCTGCTCTCTGCCTTCGGCTGCC
TCCCACACGCTCCCATGTGTCTCCCAGTCCCTAACCCCTAGTCCT
GCTTTCAATACCGCAGGGGCTTTTAGCTGACTTTTCTCCATCTTC
TTAAAGGGCAGGATTTCCTGGCAGAGACATAACGTCTTTCTGGAA
ATGAAAGAAGCTGATGCATGTTTTTTCTTTTCTGAATTACTTAAA
TAAG

In some embodiments, the intronic domain that promotes trans-splicing primarily in the liver comprises or consists of sequences from the gene RBM34. In some embodiments, the sequences from gene RBM34 comprise or consist of:

(SEQ ID NO: 523)
ATGAAAGCCTGCAATGTACTTTGGAGGGTGGATGGGTGACTTATT
TACTGTGTCAGCAGAATAGACTGAAAAGTTAATACTGATTATCAT
TGCTGGAATCCTAGGCATTCTCTTCCTATGTCCCATCATACACAT
TAGTATGGGAGGTGGGTATGATTGCTGCAAACAGTCTTTGTTCTC
TGAGCACAGACAGATAAAGAATTTATGTCAATTTAATATTAAAAC
TTTATAGAAGTGAGTTGGATTTTACCAGCTCAGCACTCTGAACCA
GTTATAGCCACATTCTTTGGAGCAGGAAATAAGTATCATTTATAT
TTGGTTTTCCCATACATGATTTTTTTTTTTAACTGCATAGGCATA
GTTAGTTGACGTACCATAGTGTACATTTTTGGGCACTATTACATA
ATTAATGTATATTAGAATATAGGAAATGTTTATTCTATTTTTTTT
TTTGAGACAGAGTCTCGCTGTGTCTCACCCAGGCTGGAGTGCAGT
GGTG

In some embodiments, the intronic domain that promotes trans-splicing primarily in the liver comprises or consists of sequences from the gene RBM34. In some embodiments, the sequences from gene RBM34 comprise or consist of:

(SEQ ID NO: 524)
CCTACTTTCCTGAAATGTTCTTAAAAACTGGCAGTAGGCCGGGTG
CGGTGGCTCATGCCTGTAATCCCAGCACTTGAAGAGGCTGATGTG
GTTGCATCACCTGAGGTCAGGAGTTTGAGACCAGACTGGCCAACA
TGGCGAAACCCCGTCTCTACTAAAAATACGAAAATTAGCTGAGCA
TGGTGGCGGGCGCCTGTAATCCCAGCTACTTGGGAGGCTGAGGTA
TGAGAATCACTTGAAGCCGTGGGGGCGGAGGTTGCAGTGAGCTGA
GATCACACCACTGCACTCCAGCCTGGGCGAAAGTGCAAGACTCTG
TCTCAAAAAAAAAAAAAAAAGCCTGGCAGTAGAGGATTATCAGAC
AATTTTTCTTAATTCCTTTTTGTAGCACTTATTTGATGTTCTGAA
GGTCAGCTAATGTGTATTTTTTTTTTTAATTTTAGAAGCTGAAGT
CGTTTTTTAAAGAGTATGGACAAATAGAATCTGTACGATTTCGTT
CTCT

In some embodiments, the intronic domain that promotes trans-splicing primarily in the liver comprises or consists of sequences from the gene KMO. In some embodiments, the sequences from gene KMO comprise or consist of:

(SEQ ID NO: 525)
TCATGAAATTTAAATGTAAGACCTTTTTGTCCAGTATAGATAAGT
TCTGAAATAAGGCAAAAGATAAAGTAAAAAATATATATATAAATA
TATAATGTATGTGCATACATTATATATGTGTGCATATACACACAT
GTACACATCTAGATATGTGCATGTGTGTGTACATATATACCAGGT
ATAATTATACCAGCTATGATTCAAGATTTGAGAACATAAGATGTT
CTCTCTGTCTTTTTCCTTACATATACACACACACGCACGTATTCT
GAAATCTTGAAATATACCTGGTATCAAAAACCTACCTAAGGTTGA
GTTTATAAAGACAGAACACAAAGTGTTTTCTAAGACAACTTCTAT
TTTATGTAGCAATAGATGTGCGTATCATCGTGTAGATGCTACTGG
TTTTAATTACTGTGGAAACTATTATAAAGCTGAGTCCGTTTTATA
GGTTTGCTATATGAATGCACTAATCAATCATATATTCTTTTTTCT
GCAG

In some embodiments, the intronic domain that promotes trans-splicing primarily in the liver comprises or consists of sequences from the gene KMO. In some embodiments, the sequences from gene KMO comprise or consist of:

(SEQ ID NO: 526)
TAGTATAGAATTTTTTATCAACTGTAATTTTGCATTTGTAATTAA
TGTTGTTTATTACTTACATTTGTACTTAGAATGTATCTACCTAAT
TGATCTCTTCATTGTTTAATCCATTTAGTGATTATAGCTTTGAAA
CTTTGGGATTACTGAGGAAAAATCTGAAGGATTTCCAATTATTTA
CTTTGGAAAGGAGATTTAACTTACATGAATGGATAACAAGAGTTT
ATGTGTGGTGGTGCCTGTCATCTCCACTAAAGACAATTTAGGAGG
AAACAGCCCTTCCAGTAAAGCCAAAGAGATTTAGGTTATCTATCA
GGAAGTGATTCAGCCTTTAAGGTCTGTAGTACATTGGAATACGCA
TTATCAAAAGAGAGCATGGGGATGGGCGCAGCGGCTCACACCTGT
AATACCAGCACTTTGGGAGGCCAAAGCGGGAGGATTGCTTGAGCT
CAGGAGTTCAAGACTGATGTGGCCAACATAGCAAGACCTTGCCTC
TACT

In some embodiments, the intronic domain that promotes trans-splicing primarily in the liver comprises or consists of sequences from the gene RMDN2. In some embodiments, the sequences from gene RMDN2 comprise or consist of:

(SEQ ID NO: 527)
ACTTGTCCTAGGAATATCACCAGTTGTTTTTGTTCTTGAAAAGAA
AAGGATAGAGAAAGAAGACTGAGGAAAATGATTATGTAAAGAAAT
CTGAAAACCATAAAGTATCTCATTGCTTTGTAAGCTTTATGACCA
TGGTAATCAATTTTATTTTAATGGCTAAATGCAAAGGCTATGTTT
CCACCCAAAAGATACTTAAACTGATCAAGTGATTCAAATGTGAAA
CAACCAACCTAAATTCTTAGGGCAGTTTGGTTCATAATATTTGAT
TTATTTAAATATATTCTCTTCACTTGTGGTTACTGTTATTTTAAT
ATTGCCCTGTTATTCTCTCATGTTTTTCTCTCAAATCAGGCTGAA
GAACTATGCCCTGGTTATTCTAATCCCAATTACATGTACTTAGCA
AAGGTAATGAAGACCACTGTTCTGCTTTAAGATCACTTTGAACCT
ATCGCATAAAAACGGATTATGTTTAATATTGGTTATTTCTCATTT
CCAG

In some embodiments, the intronic domain that promotes trans-splicing primarily in the liver comprises or consists of sequences from the gene RMDN2. In some embodiments, the sequences from gene RMDN2 comprise or consist of:

(SEQ ID NO: 528)
TAAGTCCAGAAAGTGACAGTGAGTGCTGTTGTCTTGTTAGTACTA
TTCGAGCTCAAAACAGGAATAACTCGCTTAGATACATTTGTAGTT
TTCTCTATTCAATTTTATGTATTTATTTTTATTCTACCTTTATTG
AAGTATAATTGACAAAAACTATATGTATACAGCATGATGTTTTGA
TAGACATATACATTATGAAATGACTACCACAATCAAGCTAATTAA
CACATTCATCGCCTCACATAGTTACTTTTTTTATTGTTGAGAACA
TTTGAGATCCACTCTCTTAGCAAATTTCAAATATACAATACAGTA
TTATTAACTACAGTCACATTGCTGTCCTTTAAATCTCCAGAACCT
ATTCATCCTGCATAACTGAAACTGTGTACCCTAGACCAATATCTC
CCCATTTCTCCCACTCTTCCAGCCCCTGACAGCCACCCTTCTACT
CTCTCCTTCTATGATGGAGTTTTCTTTCTTTTTTTTTTTATTATA
CTTT

In some embodiments, the intronic domain that promotes trans-splicing primarily in the liver comprises or consists of sequences from the gene SPTBN1. In some embodiments, the sequences from gene SPTBN1 comprise or consist of:

(SEQ ID NO: 529)
GCTCGCCGGGCCGCCGCCGCGTGGTTGGCGCCAGCGCACTGGGGC
AGTCGCGCGCGCCCAGGTGCGGGCCGCGTTACCTCAGCAGACGCT
AGAGAGTGAATGAGCCGCGCGGGCCCGGGACCCTTGGGGCTCTTC
ACTCTCCAGGCCGTCCCGTGGGCGCGCTAGCCTTTTCAAGTGATA
GTAATCGGAGACTTTTCCGTTACATGAGGCTCAACAAAAGATTGT
AATTCCAGCAGCTCTGTTTGGGAAGGCACTACCGCGGCGAGTCCA
GGGCCCGGCCGGGGGTCGGCGGCTGCCGGGCGGCTGGGGCGACCG
CGGACCGTGCGGGACCGGTAGGGGGTCGCGGGCCGGCTAGGCTCC
CCCGCGCCCCTCCTCGTGGTATAGCCTGCATTTCTAATACAATGC
TGTTATGCTAATCAGGTGACATCACCGCCCAGCACACGGCGAGTG
GCTCCTGATAAAATTACAAACCGGCGGCCGCCGCGGGCAGCTGGG
AGGA

In some embodiments, the intronic domain that promotes trans-splicing primarily in the liver comprises or consists of sequences from the gene SPTBN1. In some embodiments, the sequences from gene SPTBN1 comprise or consist of:

(SEQ ID NO: 530)
TAAGCCCCCTCCCAAAGGCCGGGCCTGTCCTGGGTGCCAACGGGG
GTTCTTGGAGGCTTTATTTGTGACCCTAATGAAGACGGGCGGCTT
CACAGCTCGGCCCCAGACCCTGTGGTTGGCTGCGGGCACCCCATT
CACGACTTAGGGAAGGCAGTTGCTCATACTCCACTCCAGAAGAGC
TACCATTTGGAGTCCCTGTCTCTCCAGCACCTTTTTGGCTTTCTA
GGTTGTCAGATAACTGCTGCACACACATTTTCTTTACTTTTAGCT
AACTTTACCTCCTAGTAGATCAGTGCTCATAGACTTATTAGTATG
CAGGTATTTGATTCCGGGTCGCCTCTGGCTCCCAGCCCCCAGCCT
CTTACCTCTGGGAAGCTTTAAAGGTGTAAAGTGCCGTGCTCAGAC
CCAGTCCTGTAGAACAATAGCAAAGGGGAGGAGGGAGGGGTCTTT
TTGTTCCTAAGCCTTTGGTAAATTACAGTTTTTTATTCAGAAGCT
TAAT

In some embodiments, the intronic domain that promotes trans-splicing primarily in the liver comprises or consists of sequences from the gene NFU1. In some embodiments, the sequences from gene NFU1 comprise or consist of:

(SEQ ID NO: 531)
GTGGGTAGTGCATCTGATGGAGCCGGCGGGTCCGCCCTGTCGCCG
TAGTTGGGGGTGAGCCCTGAGGCTGTCTGTGCGGGAGCTCGGCGC
TTCTGTGGCCTAATCCGGGCGGACTCTCTGGAGCTTCCTCTCAGG
CTCAGAGACCCAGTTCTTGAGTCTCTGCGGTACGGAAGCTTGTAG
AGACTGGCTGTAGAGCCAGAACGGAGGAGCTGTTTTAAAATCGTG
TGCCTCTCCCGCCCCCAGGCCCTCTACCGGGTGCATTCCCTCCTC
TTCTAAACGCAGAATCAGAATGTCACACAGAAGGGAAGAAAATAC
CTTGTCTCGTTTCCTCATTTTCCCAGTAAGGCCCTGAAAGAGGAG
GTGACATGTCCAAGGTTACACAGTTTCTAAGTGATGGAATTCCGA
CGAGAACCAGGACCCCTAGATTCCCTGCACCCACCACCATTAAGC
CCACTCTGTTCCCTTGTTTTTTCCGCCTCCCTGAGCCCTACCTTT
GAGT

In some embodiments, the intronic domain that promotes trans-splicing primarily in the liver comprises or consists of sequences from the gene NFU1. In some embodiments, the sequences from gene NFU1 comprise or consist of:

(SEQ ID NO: 532)
CGAGAACCTTTTAGAAGGAATGCTCCAGGGAGTCTTTGGGGGCAG
TCACTGAGCTGGGCCTGGAGGCTGAGTAGGGTCCTAGAGGAGGGA
ATGGGTCAGGTTTAGGGGCTGGAAACAGCATTCGCAAGGCAGCAC
CAGCACCAGCTAAAAACCTAGCCAACAGGGAAAACAAAGTACGGA
AACACGCATGCGTTCACAGTGCTGCGTGAATTAACCAAGCGTTGG
CGCAAACGGGAAAGGTTCCCCGGCCTCTCTTGGTCAGGGTGACGC
AGTAGCCTGCAAACCTCGGCGCGTAGGCCACCGCACTTATCCGCA
GCAGGACCGCCCGCAGCCGGTAGGGTGGGCTCTTCCCAGTGCCCG
CCCAGCTACCGGCCAGCCTGCGGCTGCGCAGATCTTTCGTGGTTC
TGTCAGGGAGACCCTTAGGCACTCCGGACTAAGATGGCGGCGACG
GCCAGGCGGGGCTGGGGAGCTGCGGCTGTTGCCGCCGGGCTGCGC
AGGC

In some embodiments, the intronic domain that promotes trans-splicing primarily in the liver comprises or consists of sequences from the gene PCBP1-AS1. In some embodiments, the sequences from gene PCBP1-AS1 comprise or consist of:

(SEQ ID NO: 533)
GTTGGTTGAGGGGTTTAACAAGGCTAAGAAGGTGAGTCAGGTTTA
GACTTTGGAAGTTGAACCACAGGATTTATGCTACATACAGAGGAA
TGATTGTTGCTTCCTCTTATGTTAAGTTGGTATGTCACGTAAAAC
CTTTCAGAGTGCTTTCACGTTACTTGTTTAATCATCACAGACAAC
TTTGCCAGTAGGGTCTTATGCCCATTTTACAGAAGAGCCATTTTT
GGATTAGAGGAGTGAAGTGACTTGCCCAACGTTACACAGCCTGTT
AGAATTTGAGCTGAACCTCTTGACTCTTAGTTATGGATTACACCT
TTCCATGGCTTCTGATGCTTTTGATATGGACTTTGATGTGTGGGA
TAGCTTTACCTTAAACTCAAAAGGCTTTCTAGCCAGTTACTATTT
TTCTGTCCCCAAACAGCCAATGCATTTACCTTAAGTTCCTAACTC
TTGTTTTAGAAAATCTCTTCCCTCTTTTCCCTGATGTTTACATTT
ATGG

In some embodiments, the intronic domain that promotes trans-splicing primarily in the liver comprises or consists of sequences from the gene PCBP1-AS1. In some embodiments, the sequences from gene PCBP1-AS1 comprise or consist of:

(SEQ ID NO: 534)
CTACCTACTGAAATATTTCCATAATTTCCTGTTATGTACTCCTGG
GACTTAGTGCACCTCCTCAGTGAAGCCTTCCCTTTCCCAGCTTCC
CCAGCTCTTAGCCTCCCTCCTTCCCCGAGAGTCTTAACAGTTTCT
CCCTTCGGTAGCCCACGTTTGGACGTTGGTTAAATTGTAATGTGT
CTTGGGGTTGGTAACATGTCATACAGACACCTTGGGTGATGCTGT
GGTCTGGCAAAGAGGTCATTGGACCTTGGAATCTGAGGCTCTGGG
CCCTAGTCCTAGCTCTGCCCCTGTCTAGCTCTGAGACTTCTACAA
CTCATCTGTCAGGGTTTCAGTTCTCCCTTTTATAAAGCAAGAGAT
TAAATGATCTCTAAGGGTCCTTCTACTTATGTCCAGGGACTGATG
ATATTTGATGACTTTGGTTATTTTTCTTTGGCGGGGTGGGAGGTT
TATGTAAATGTTTTCTGCACTTTTGGTAAAATCTCCACTCCCTTC
CTCA

In some embodiments, the intronic domain that promotes trans-splicing primarily in the liver comprises or consists of sequences from the gene IL1R1. In some embodiments, the sequences from gene IL1R1 comprise or consist of:

(SEQ ID NO: 535)
TCCTTAAGTGAAAAATCTGTGAGGAGTTTTCTATCTTTCTAAGTC
TTTGTGGCACACTTATCACATGCATAGTACTATTTCTCAAGTTAC
CCAGGCACACACAGATGTATGGCTTTCAGGAAGCCATACATCTGT
GTAGCTCTGAGGCCCAGCCTTGTTCAACACTTCACTCAGAAGAAT
TGCTGGCCTGATGACCAACAATAATTGTCAACAACTTAAAACTTC
ATTGACGGCTCCTAAAAAAAATCAGTGTGCATGTGTGTGTGTATG
TGTGTGTGCGTGTGTGTGTGTGTGTGTGTATGAGTGTGTGTATAA
CATTCATTACTGCAAACTGGAATGGAGTGGGGCTGGAAGGCAGCC
CAGAAGCAAGGCTCAGAGCCTGGAGCTGCTGACCTCTTGGGGACA
CTGCAGCCCTGGCCTGGCCTACTTTTCTCTTCTCCCATATCTGGA
AAATGAAAGTAGCCTGACGTATCCGGGGACCCGGCCCAAGACTCC
ACCC

In some embodiments, the intronic domain that promotes trans-splicing primarily in the liver comprises or consists of sequences from the gene IL1R1. In some embodiments, the sequences from gene IL1R1 comprise or consist of:

(SEQ ID NO: 536)
TAAGGTTTTAAAGGGCCCGCCCTGGCTTCCTTGGGCCTGTAGTTT
GTGACTGTGTCGTTTTCAGAGTGGGAAGGCCCAGAGTTGGCCCCA
TGGAATTCTTTCTGCTTCTAGGGTGTTTGAAGAGATAGAAAGCCA
GGGTAAGGTTCACCGAACCTCCAAGCAAAAATAAATTTCCAGAAG
GGGCCCCTTTACTCTGAGGACATTGAATAGCAACAAAAACTACCA
CTGAAGAGTCATTTTGTCCCAGGCCCTGTGCTGGAAACTTTGAAC
ACCTGTGTTCACCCCTCCCTCATGACACTCATGAAGATATACCAT
GGCTGTCTTTATTTTCTAGGTGGGAATCTCAGGCTTGAGGGTTGG
GGTGGCTTGGGGAAGGTGTGTATCTAAGAGGTAGCTCTTATTTAT
TTTTACTTCTAGAGATTAAGACACATTCTTTTTCAAGATAGACAT
GGAACTGTCAAAAAGGATTAAAAGTTAAATAAGGATAAGGTTATG
TAAA

In some embodiments, the intronic domain that promotes trans-splicing primarily in the liver comprises or consists of sequences from the gene DBI. In some embodiments, the sequences from gene DBI comprise or consist of:

(SEQ ID NO: 537)
AGCGCGTGCACAGCCAGGCTGCGAAGGTGCAGCGGGCGGGAGGCC
CGTTGGGGGCTCAGCCGGCTGCCAGAAGCTCTCGGGCTCTTTCCT
TCCGTGCCCCTCACTTGCTCATGGGCCCATGCCTAGCCCTGATTC
GTTGGACAGAGCCTTGTGAGCGGGATTTTCCGTTTGGGGATTTCT
AAATCTGCTGCCCACCCCGCAACTGCCGGAAAGTTGCCCATGGGG
TGGACTTCGCTGTGTAGCGGGAGAGGGGTGGGAGTCGAGGGTGCT
TGATGGAGAGATGGGGGAAGGGGTTGCACGGATTGGAGGAGCGAG
GAGACTCAGTCCCCATCCCGAAGCACAGGGCAGGACGTCGCGGCG
GAGTGGGGAAGCGAGGAGTCCGTGGCCGAGAGCTTGGAGGTCAGG
GGAAGTACGGGGCCGGCTGCTCAGAGTGCGGGACGAGGAGAATCG
CGGCCCGGGGAGAGGTGACCCAGGGGCCCCTCCCTTCTCTCCAGT
GTAG

In some embodiments, the intronic domain that promotes trans-splicing primarily in the liver comprises or consists of sequences from the gene DBI. In some embodiments, the sequences from gene DBI comprise or consist of:

(SEQ ID NO: 538)
TCGGTGTTGAACGCGCGGGCCCCAGGGGGAGGGAGGGGACCAACG
GGCTCCGGCGCTGACACCGCGGCACTCATGCCCTGTCCCCTTTCA
GCTGTTTCCAGCATACTGTGCCCCGTCTGTCCTCAGGCCAGGGCT
TCGCTGCAGCCCCGGCCACTCCCTAGTGCCTGGCCCGGTGGTGGC
CAGGCAGTTGGCCGCGCTGCTTCTCCCGCAGAGGGGACCCCCACT
GGGGGCGAAGGCTTGGCCTGCCCTCTTCACTGCTGTATTTCCAGA
CCTGATGCCTGCGTTTGTGAGAGCTCTGGATATATGGTTTTCGAT
TGAATGAGTGAACTGGAGGGGCTTCCCCTTCTTGTGTTGCTGAAT
CTTTCTAGCTGCCCTGTTGGGGCAGGGAGGGGCAGACACACTTCA
GGGGCTGCATTGCCCGAAGGGTGCCACCTTTCCCACCTCTCCATC
CCCGTAACTGGGCTGTCATCAGGCCACAGTAGGATTCTTACCCTC
TCCC

In some embodiments, the intronic domain that promotes trans-splicing primarily in the liver comprises or consists of sequences from the gene HNMT. In some embodiments, the sequences from gene HNMT comprise or consist of:

(SEQ ID NO: 539)
TCCCTAGAGTTGACTATTGCCTTGATTTTGGTTTCTTTTCTTAGT
TCTCCATGTACCTATTACTATTTTGATCCCCAAGCAGATATATTA
ATCTCCTCTCAATATATGCAAACATATCAAGGAATCTGTTTATTT
TACCCCAAGGGAACAACTTTTCTGAAGCCCTCTGTTCTTTTTTTC
AGTTTAGGAAGATCTGCTTTGGCCTGCTATACAACTATTAAGGAG
GGAATTTATTTCACTTTCTGAGAATCATTTTCTTTCTCCATTATT
TGATTCCCTGATTCTATATCTCATGCCTTTCTTTTCTTTGTTTCC
TCGGTCCTTTGTTTCCTGAACAATTCTTTAAGAACTTTAAGAGTG
AACGAGAGGTAAACTTTTTGAGACTTTGTATGTTTAAAAATCCCT
TGGTTCTATGTTAATCGTTTGACTGGATATAGAACTCTATACTGA
AAATGGTTTTGTATAATAGATCTAAAGGTACTTTTCCACTGTCTT
TTAG

In some embodiments, the intronic domain that promotes trans-splicing primarily in the liver comprises or consists of sequences from the gene HNMT. In some embodiments, the sequences from gene HNMT comprise or consist of:

(SEQ ID NO: 540)
CCTTGGCATGTGGAATAACTTTTACATTGCTATGTGTTTTGTTTT
CCCTATTTTGAACTTTTTAGAATCTTCTTACGTCTAGTATTCTGA
AATTTTAAAATACTGAGCTGTGAAATGGTTCCTTTTTACCCATTT
TATGGGTACTCCCTGGGCTATTTCAATTGAGATTCAGATCCTTCA
GTTATGCGGAACTTTGGTAATCATTCTTCACCACTGTTTTCTTTT
TCTGGAACTTTTATTCATCAAATACTTCCTGGATTGATTACCTAA
TTTTCTTATATTTCCAGTTTGCTACAACTTTCCTCTTTCAGCCCA
TGTATTAACTTTTTACTATTATATTTTTAATTTCCAAGAATGTTT
TCTATAGCATTCTTTTCATCATTCCCAGATGTAATACCTTCTTAT
TCATTACAAAATGTTCCTCTGCTCTCTCTGCCTCTTTCCATCTCT
GTTTCTTATGTTAGACAGCTCCCCAACCACCTAAAGAGCCTTGGT
TTTC

In some embodiments, the intronic domain that promotes trans-splicing primarily in the liver comprises or consists of sequences from the gene KYNU. In some embodiments, the sequences from gene KYNU comprise or consist of:

(SEQ ID NO: 541)
GATCTGCCCACCTTGGCATCCCAAAGTGCCAGGATTACAGGCGTG
AGCCACACACCCGGCATGTTTTCTTTTTTTGGAAACAATAACAGG
ATATCAGATTGATAAATGCAATACCAATTTCTGATAATTTATCTT
GCTATATAATACTTCATGGACTTCTTGCACTTTGTGGTGTTTTAT
TAGTCAGGAATAAGGAGAAAACAACTTATAGCTGTGGAAAGAGTA
CAAATTAATTTTTAAAAACTGGAGAATAAATCAAGCAATTCGGCT
TTTGAAGACTTTCACTTGAAAAAACACTTTTTACTTTTTAATTAT
CATTTCTTAATAATAAAAATTATGCTCAAATGCTGCTCTTATGCC
AGATTTTTAAAGAACCTTAATCTGAAAAAAAAAACTATTTTATAA
TGCAAAAGGGCTCACGGTGAAATTTAGATCGGCAATATGAAATTA
CAAATTTATTATTATCGATATGACCTAACTTGTGCCTAACTTGAT
TTAG

In some embodiments, the intronic domain that promotes trans-splicing primarily in the liver comprises or consists of sequences from the gene KYNU. In some embodiments, the sequences from gene KYNU comprise or consist of:

(SEQ ID NO: 542)
TATGCACGCCATTTACTTCTTCCCACCTTACTCCAAACATCACTC
TACTTAAGAGTGTTCTAATTGCATGACTTGTGAAGTACTTTAGCT
TGTGTCTGAGTAAAACTATTTCAAAAGTTAAAAAAAAAAAGAGTA
AACCTTGGGCCAGGCACGGTGGCTCACGCCTGTAATCCCAGCACT
TGGGAGGCTGGAGCAGGGGGATCACGAGGTCAGGAGTTTGAGACC
AGCCTGACAAAAATGGCGAAACCCCATCTCTACTGAAAATACAAA
AATTAGCTGGGCATGGTGGTGGGCACCTGTAATCCCAGCTATTCG
GGAGGCTGAGGCAGAAGAGTCACTTGAACTCGGGCGGTGGAGATT
GCAGTGAGCCAAGATCGTGCCCCTGCACTCCAGCCTGGGCAACAG
AGCAAGACTCCATCTCAAAAAAAAAAAAGCGAGTAAATCTTACAT
TTTTAAAAGAAAATCTGTAATCTTTTTTGCTTCAGGCATCTCTAT
TTAA

In some embodiments, the intronic domain that promotes trans-splicing primarily in the liver comprises or consists of sequences from the gene GRB14. In some embodiments, the sequences from gene GRB14 comprise or consist of:

(SEQ ID NO: 543)
GTAAGTTTGGTATTTTTAATTTTGAGTACTTGTGAATTGGATTTC
TCCTATAAGCAGAAATTATTTATTCCATAGCCACAAAGAGTTCTT
GATTTGGGCTTATCATAAAACAGAAGTGACATTGTTCTATAAATG
AGGTCATCACTGTATTCTAACAGTATCTATATTATACCTGGTACA
TAGTGTGTGTCTGGTATTTGTATATAAATGGACAAATAACGGAAT
AGAGTATCTTGTTCTTAACCAGTGATCACTAATATTACCCATTAT
GTCCTGATTCTAGCATCTCAGATATCAATTGATGGGGGAAAATGA
ACTTCTTTTTAAGTGTCAATATTTTGATTTGAACATATGATTTCG
AAGTATGGTTTATATTATGACATTATGTCAAATGTATTTTTTTAG
GTATGATGCAACATGTATTAAGTAAAAAGCTGTTTCTTAAGTGTT
TTGAACACTTATTTTTTTGTGTGTTTACATCCCTATTGTCCTTTC
TCTA

In some embodiments, the intronic domain that promotes trans-splicing primarily in the liver comprises or consists of sequences from the gene GRB14. In some embodiments, the sequences from gene GRB14 comprise or consist of:

(SEQ ID NO: 544)
AAAAACACTGAGAGAAAAACACTGAAATTGAAAAACACTTTCAAT
TTATTTGAAAGATAACTTTCAAAGAAATTTTCATAACTCAAATGT
GTTTCTTTTTTCCAGAGAAGTATATCAGAGAATTCCCTGGTAGCA
ATGGACTTCTCAGGCCAGAAAAGCAGAGTTATAGAAAATCCCACT
GAAGCCCTTTCAGTTGCGGTTGAAGAAGGACTCGCTTGGAGGGTA
CAAGTAGCTTCACATGCTTCAAAATATTTATGATTTCAGTTTGTT
TCACCTAAAAACTTGAGATTTGTTTTCTCCTTCCTTGCTCAGAAA
AAAGGATGTTTACGCCTGGGCACTCACGGTAGCCCCACTGCCTCT
TCACAGAGCTCTGCCACAAACATGGGTATGTCTGTTCATAGTCAT
TGCCACTGAGTAGACGGATAGACATGGAAAGGACATGTAATGAAA
TTGCAGTCATCTCAAACAAAGCTTTGCATTCATCCATCCTATTTC
TTTA

In some embodiments, the intronic domain that promotes trans-splicing primarily in the liver comprises or consists of sequences from the gene SPATS2L. In some embodiments, the sequences from gene SPATS2L comprise or consist of:

(SEQ ID NO: 545)
TCAGAAACAGTTATTGGTATTGCCAAAAAAAAAAAAAAAAAAGGA
AGCTAAGAAGTCACCCTTCTAAATAAATAAATAAATAAATAAATA
AATAAAAACCTGAATTGGAGAGCTGCCTGCATTTCACTCTACTGC
CTCTGGAAGAAGAGTGTTATCTAAAGTGCTTCCATCATGAATAAT
GATTGAGAAACTGCTTTTACTCTCCCGACTACTGTACTCCCAGGC
AGAAACCCACGGCTGAAGCCCACGATGCCCATGTGTAACTTGGTT
ATTACACACACGTTTGGAGCCCAGTGTGCAGACCCGCAGCGCAGA
GGAGGCGGCGGAACACCCCGGGTTGGTCGGGTTTCCAAGGGCTGA
CCCGAGCTCCAGCACTTTTTCCGCGCCTGATTTTTCAGGTCATTT
TCGAATGGGCTATCTTTGCTTACATTTTGAGCTTGGATCCTCTCC
AGAAAGCATTACTACCTTCTAAAAGGAAAAACATCTCGTATTTGC
AGAC

In some embodiments, the intronic domain that promotes trans-splicing primarily in the liver comprises or consists of sequences from the gene SPATS2L. In some embodiments, the sequences from gene SPATS2L comprise or consist of:

(SEQ ID NO: 546)
TAGGAGAAGATGATTCTCTTGCAACACGTGCGGATTGTGACAAAA
TCTTTCATTAACAAGGGGAGTTTCGGTGAAGTGGAGGTTTGGGGA
AAGGCGAGGAAGTCGGTCTGGAGCAAGCAAGCAAAGTGCGGAAGC
TGTACTGGGATTCTTCTAGAAAGTGGGGTGGGAAAGGAGCTAGGG
AGGGCGTGTGGAGGGACGAGATCTGTGTCAGAACGTGCGTGTGAG
CGGATACAAAACCCGAGAGAGGCGTGAGCAGCGCTGTGTTTGCGA
GCGGGAGCGAGGGGCGCCGGCTGGGGTGTGTGCTCCTGGTGAGTG
ACACCGAGGGGAAGGCGGGCGGGTCGGCGGGCGGGGGCAGCCTGC
GAGGGGCGGGAGTGTCCCTGCGTGGGGCGGCCGAGCCGGAGTTGT
GTCAGTGAAGGAATCCAGTCCGGGGGCCGAGCTGGCTGCGCCCTC
CGCTGCAAGCGCCGGCAGCGCGGGGCGAGCTCCGGACGGCGCGCG
GCCC

In some embodiments, the intronic domain that promotes trans-splicing primarily in the liver comprises or consists of sequences from the gene CPS1. In some embodiments, the sequences from gene CPS1 comprise or consist of:

(SEQ ID NO: 547)
AGGGCTGAGGATCGCTCCATCTTCTCAGCTGTCTTGGATGAGCTG
AAGGTGGCTCAGGCACCTTGGAAAGCTGTTAATACTTTGGTAAGG
AGAGAAACAAGTATCTGTTTCTAATGTTCTATTTTGAAGAGCTGC
AACCTGAATGTTGACTGAATGTTGACTATTGGATATGTTAACTAC
AGGGCAGATAGAATGCATACACTTAGTGAATTTAAAGTTGTTGTA
TATCCATGATTGCAGTTAATGCTGATTATGAGTTGATAGCAGTAT
TCTCATTAAGATTCAGACAATGAAAATAATTTCAAACCAGTTTTG
AACAAGATTTGATATTTAGAATCAGAAGCTAGGAATTTTATGTAT
TTCAGTGTCCAATTTACATTTAATGGTGCCTTTAGAGAATAAAGA
AGCTGGGCTACCACTCGAGCTAATGATACATTCTGTAATTTTATT
GCATATTTTAAACTACTCATACATTTTTATTGTTGTTTCTATTAA
TTAG

In some embodiments, the intronic domain that promotes trans-splicing primarily in the liver comprises or consists of sequences from the gene CPS1. In some embodiments, the sequences from gene CPS1 comprise or consist of:

(SEQ ID NO: 548)
TAACACTGTATATTGTTTTCCAAAACAATGATTCAAAAATTGGGA
GATACAGAAATGCATGCTGTATGCTAATAGCACTAGGGTTCTATA
TGTAGTAATTTATAAATCCATTAACAATTTCCAAACCATCACAGC
CAGTTTAAGGACCAATGTGTGGTCTTCTAGTTTAATGCTTTCCCT
TCAGGGAGTGGCCATAGGAAGACTACAGTTCTTCATCTAGAAGTG
TAGTGTAAGGCATCTCCTTACCAAACAGAGAGGGTAGCTTTATAT
TAAAGAACAGGACAATTTAAAAAAGACACACAAATTGAGGTATCT
CTCGCCAGCGTGTTTTCTGATACCCAGAAATGACAGCTTATCTTT
GACTATTGTCAGCACCTTCTGATAGTGCCTTTGTTTTAAAATTTT
CAGTTTTTGAGATCCGATTGTTTAATAGATAAATTATAAGGGTGA
GGAACAGGAACAGTGAGGATCTTTTATATTAAACTTTAAAACAAA
GTTT

In some embodiments, the intronic domain that promotes trans-splicing primarily in the liver comprises or consists of sequences from the gene PECR. In some embodiments, the sequences from gene PECR comprise or consist of:

(SEQ ID NO: 549)
GTAGGTAAACGATCTATATTGAAATTTATTGACAAATTATGCTTT
CTGATTCCATTTGTGATGTTGATAGAGGTATTTGGTAATAAGCAA
GTATAGCATAAAAGATTGACTTTAAAACATAGATAAAATGGAAAG
CCTAATTTTCTTTTTTTTTTTTTTTTTTTTTTGAGATGGAGTCTC
TGTCGCCCAGGCCGGAGTGCAGTGGCATGATCGTGGCTCACTATA
ACTTCCACCTCCAAGGCTCAAGTGATTCTTGTGCCTCAGCCTCCT
AAGTAGCTGGGACTACAGGCACAAGCCACCATGCCCAGCTAATTT
TTTGGATTTTAGTAGAGATGGGTTTTCACCATATTGCCCTGGGTG
GTCTCGAACTCCTGAGCTCAGGAGATCTGTTCGCCATGGCCTCTC
AAAGTGCTGGGATTACAGATGTGAGCCACCGCACCCGGCCCAGAA
GGTCTAATTATCATTATGGAAGTACTCTTTGATTTACAGCCAGAT
TGTC

In some embodiments, the intronic domain that promotes trans-splicing primarily in the liver comprises or consists of sequences from the gene PECR. In some embodiments, the sequences from gene PECR comprise or consist of:

(SEQ ID NO: 550)
CCCGGTAATTTTTGTATTTTTAGTAGAGATGGGGTTTCACCATGT
TGGCCAGGCTGGTCTTGAACTCTTGACCTCAGGTGATCCGCCCGC
CTTGGCCTCCCAAAGTGCTAGGATTATAGGTGTGAGCCACTGCAC
CTGGCCTCCTTTGTTATTTAAAATCGATGTTAAAAATAACCACAG
GCTTGAAATCCTTAAAATTACCAATGTGTTTTCCTTTGTAATCAA
AGTTCTTTAAAGTATTCCTCTTACGTAGTGATTTGGGGAGCCTTA
TCTGGGTCTATGCCATGTGAGATTTGCTTGATCATCAGCTGTGGC
TTGGATTTAATATTGAAGGACATAGTTTTTTCTTCGTTGTTTAGA
CAGAAGCTCAACAAGACAGACTCCATAGCAAGAAGTCTCTTGTGG
AATTCTGTCAGGTAGTTGGCACACTTGTGAGCTTGATGAACAAGA
GAAATATTCAACAGAAGCTGATTGTCATGTTTATGTTTTGTCTTT
CAAA

In some embodiments, the intronic domain that promotes trans-splicing primarily in the liver comprises or consists of sequences from the gene SP100. In some embodiments, the sequences from gene SP100 comprise or consist of:

(SEQ ID NO: 551)
GTAGTTATTTTCAAATCTTTTCGAAAGGAAAGAAGGAAGGAAGGAAAGAAA
GACGCAGGAATATGTATAAAGGTATAGCAAACATCACACTTAATGGTGCTG
CTAAGTGCTTCTTCCTTGAAATTGGAAATAAGGCAAGGAAGTCCACTCTCA
TCACTTCTCTTCAACATTGTATTCAAACTCCTAACCAGTGAAATGAGACCA
AAAAATAGAAATAATTGTTTGGAAAGAAACAGTAAGTCAGTCTATTTGTTC
ATGATATAATTATTTTCTTTAAAATACAAAGGAAATCTATAAATAATTTAC
TAGAAATACTACATGAATTTAGCAATGCCATAAATTCATGGTTAATATGTT
TCAAACCAATTATATTTTCATATAATAGTAATAAGGAATTTGAAAAGCATT
ATCATTTAAAGTAACACCAAAACAATAAAATAACTAGGAATAAATTTATTA
TTTTTTAAATTTAATTAAATTAAATTTTTTATTTCAATAG

In some embodiments, the intronic domain that promotes trans-splicing primarily in the liver comprises or consists of sequences from the gene SP100. In some embodiments, the sequences from gene SP100 comprise or consist of:

(SEQ ID NO: 552)
TAAGTAAATGGAGAGATATACAATGTTAACAGATTGGAAGCTCAATGTTGT
TGTCATCTCCCCCAAATTTATCTGTAGCTTCAATTCAAATCCAAAAAAAAG
TCCTAAAGTTTTTTCTTGAAGAAATTGACAAACTGATTTGAAAAGATAATG
TATAAAAGCAAAGAATCTAGAAAATCCAAGCCAATCTTTTCTAATTGACTT
CAAGACTTTTGTTAAGCCATGATATTTAAGGCAAAATCTTACCAGCATAAG
GGTAGACAAATAGATCAATGGTATAGCATAGAAAGTACATATATAAACCCA
TGCTAATATTGTAAATTGTTTTTTGAATATTTTCATGACCTTGGAGTAGGC
AAATATTTCTTAGATAGTACACTAAAGTCATTAATTATAAAAGAAAAAAAA
ATAAATTGGATTTCATCAAAATTACAGATTTCTTCCCATCAAAAGACATGA
TTAAGAAAATGAAAGAGAAGCTACAGACTGGGTGAAAATA

In some embodiments, the intronic domain that promotes trans-splicing primarily in the liver comprises or consists of sequences from the gene TRPM8. In some embodiments, the sequences from gene TRPM8 comprise or consist of:

(SEQ ID NO: 553)
CTTACTTTAAGGAAGGTCCCTGGAAGTTGTTCATTTCCTGCATAATAGTCC
GTTGATTCTTGCATTTGCTGCACTTACGTTCTAGTTCATTAAAAGAATCTA
GGCTGTTCCTTGGGGGACTCATCAAAAACCCACATTTTTATGTTATTTCAG
TTTTAATTCAGTTTTTAAAAATTTATGCAGAAACCACTGAGCAAATAAAAT
GCTCAGAAACATTCTGGGTTTTAACCTAGATTGAATGAGTTTGAATGAGTT
GAGCCTTGATCATTCAACCTTAGTCACGCAGCTCACACCGATAAACTGGTC
AGAATACACATCTTTAAGAGAAAAAAACATTCTCAGAGCTGCTTCTATAAA
AAAAAAAATCTCTCAACTTTGGTGGTATATTTGGATCTATTCCCAGTATAT
TCCCAGTATATTTTGAAGCAAACCATATCCTGCCTGAGAATGGAAGTGGGA
AATCTATTCTTAACACAGTGTTCTTTCTTTGTTTTTACAG

In some embodiments, the intronic domain that promotes trans-splicing primarily in the liver comprises or consists of sequences from the gene TRPM8. In some embodiments, the sequences from gene TRPM8 comprise or consist of:

(SEQ ID NO: 554)
GTGTTGCTTTTGTCATTTTATTGAGAGTGACAAAATAAAATTCCTTAATTC
GGTCAGATTCACTTCTATAGGGATTTGGTTCCCAGAGTTACATGAACTGAC
TTCTCTAAGCAGATGACTAGAGCATTCTGATTAAATGCAAGCAAATGAATT
CCCCCCTCTGGTTCTGGTTCTTGTCAACATGGCCAGATCTGAATTCTGTGG
CCCACTTTTTGGTTTCCCCCAAGTCAGGCATCCATCTGTAGCCCCGGGGAC
TCCAGATTCATTGACTCAGAGCCACCCCAATGTGTCATGGGGCTTTTTTGA
GGCCTCATGTGGGCTCACAAACAGGTGCTCCTGCTCTATGTTGGGGTCAAC
ACGAATGTTGATTGTCCACAAACCACTTGACAATGGAGCTTCGGGCCCAGC
AAAGGACATGATTCCCTTGTTGTAGGCATTGTCTCCAGAGTGTTCCTGGGC
CCTCCTGCTCCAAACACACATTCATTCCAGGAAGAATCTG

In some embodiments, the intronic domain that promotes trans-splicing primarily in the liver comprises or consists of sequences from the gene SEPT2. In some embodiments, the sequences from gene SEPT2 comprise or consist of:

(SEQ ID NO: 555)
AGTACATTCCTCACATTTGTGAACAGTTATGTTTCTAATTATATGATGTTC
AAGTTCTGTAGTCTCATAGACCTGGATTCAAATCTTGGCCCTGCTCTGGAT
AGTACTCTGACCTTGGGCAAATTAGAGTTTCTTTGAGTACCCTCAAAGATC
ATACTGCAAACATAGTAGTACTGTAGGTACTAAACACAGAATGATGTTTGT
GAGGTACTTAGCCCAGTACCAGGCCTTATGTAAACACTTAACCGATTGAAT
GGTGTCATATGAATAAGGTCATGACAAGGTTTTTCTTTTTATTTAAAGGTG
AATCAGGTCTAGGAAAATCGACTCTCATAAACAGCCTATTCCTAACTGATC
TGTACCCAGAAAGAGTCATACCTGGAGCAGCAGGTAAAAACATTCTTATGT
TACTGTAAGTGTAATTCTACATGAAAGATGCTGAAGACTGCCTAATTAATA
TTTTATGTCTTAGCTGTGTGTTTTGTTTGTTCTTGTTCAG

In some embodiments, the intronic domain that promotes trans-splicing primarily in the liver comprises or consists of sequences from the gene SEPT2. In some embodiments, the sequences from gene SEPT2 comprise or consist of:

(SEQ ID NO: 556)
TAGGTGTGCTGCACCGAGGTCCTTGGATTTGGGTTGTAGACTTTCTTTGAC
ACGTATCCATGGATCCATCCTCTCTTGAGTTTGTCTGTAAAATGTAATAAG
TAGTTTCTGCCTATATTAAATATTTGATGGAAGGATTAAGTGAGAATGTTT
TACATCAAAAATGCTGTGTAACTAGAAGATTACTAGGATAGATGTTCGAAT
TCATCTACTGTCAGAAAGTTCCAGCACAGGACACCAGCATCCACAAATGAC
TGTTGAATGAAATAGGCACACATAGTGAGAGCCTAAGACTTGGGCTGCTGC
AAGAATATGTAGCCCTTTCAGAATATTTTTGAATTCAGTTTTTAAATGAAT
TCGTAAATTGCTTTCTTTTTCCCAGGAACAGAAATTATAATTTTTAACATT
TGTGTTTTTAACCTTCCCCAGTAGCACAATAATCTTAGCTTTAACCTTCCC
CTTTCTGACTTCTCTGGGATCTTTTTTTCTTAATCCCCAT

In some embodiments, the intronic domain that promotes trans-splicing primarily in the liver comprises or consists of sequences from the gene RPL15. In some embodiments, the sequences from gene RPL15 comprise or consist of:

(SEQ ID NO: 557)
CAGAGACTCCGCAGGGGGAGAGCCCGCGGTGCGTGAAACAAACCTGTTCTC
TCCTCAGACCTCAAAGACAGCGGCTCCACCGCGGTACGCGGCCACCGGCTT
TGGAGCCTGGACCCCAACTTGCCTCCTCTCGCGGAGAGACAGTCGCCGACG
CTCGCTTAGCCGCCGAGACCTCGCCGCCAACTCTCTCACCTCTCGAGACGC
CCAGGCCGCTCAGGCTCGAATCTTGCGGAGCAGGGGGCGGGACAATAGCGG
CCGCGGCGCCCCACTCGGCAGAACTCCGCCACCAGGCGCGATGCCGGAACT
ACATGTCCCATGACGCTCTGGGAGGCCGCAGCTTTCCACCGGAAAGAGGGT
GGCTGAGGTGGGGGAGGAGCCCAAAAGGCATTGTGGGAGTACAGCTCTTTC
CTTTCCGTCTGGCGGCAGCCATCAGGTAGGCTGCGTTGAGGATCTTTGCTC
TTCCATCCGCCTTTGATCGTCTTCCTCTTCAGCCATCCAG

In some embodiments, the intronic domain that promotes trans-splicing primarily in the liver comprises or consists of sequences from the gene RPL15. In some embodiments, the sequences from gene RPL15 comprise or consist of:

(SEQ ID NO: 558)
TCAGGATGCGGGACAGCAAAGGGGTGGAGAGGCGGCCCCCGGGGCGGGTTG
AGCGAAGATGTGATGGCGGCGCGAATTCGAGCTGGGCCCGGATCCTGGAGG
AGGCGTGGGGCTGACGAATGGGGCGCCCGGGACAAGGCAGCCTAAAGTGAG
GCTGCTGGTTGGGTGGGGTAGATGCATTTTCCTCGAGAGTTAATCCTCGGT
GGCCGCAGCAGTACCTTGTCCTGTGATGTCAGCGGCATCTCCTTTCAGGTC
CTCCTTGGGGACGCCGCCTGCCGCAGCCACCCGCCCCCTTGGTGCTCAGTT
CTGGTTCTGTTAATTCGCCCCACCAAAACGTGCCGAGCACCGCTCTGTGCT
GGGGTTGCGGAAGTGACTGAACGCGGCTCCGTGGGCGCAGTGGTGGGGGTT
GGGCTAGCTGTCCCCGGCAGTTGGTGCAGAGCCATTTTCATTCCCGGCGGT
TTCCTTGTTTTTGTTGGGGAACTAAGATGGACGGATACAG

In some embodiments, the intronic domain that promotes trans-splicing primarily in the liver comprises or consists of sequences from the gene AZ12. In some embodiments, the sequences from gene AZ12 comprise or consist of:

(SEQ ID NO: 559)
GTAACGAGCTCTTCTCTTAGGTTTTGGAGTCAGAGTTTGATTTGAATCCTA
CTCTACCCACTCTTCATTCAGTAATCAATATTTATTGAGCAACTTCTACAC
AGTGATGAGTTGGGGACACAAATGATGGGCAAAACAGACAAAGCTTCTGCC
CTCACAGAGCTTAACATTCTTCTAGATTTAACAGGGCTAATTCAACTTTCT
AAGACCTACCTATAAAATATAGATGATACATATTTCATAGAATTGTTCAGA
TAATTAAAAGAGATAATATTAAAAGAATGTAGTGCATTATAGTGGGACATA
TTGGACACTCAAATGTTAATTAATTTGTCTTCTATTCTAAATATACAACAA
ACATAGCCAATTACTGGTATGGAAAGATAGAGTATGCATTAAGACTTCTAT
AATATAATATTAATATGTTGTTGTAATGAATTATACTCAAAGAACTTTTGA
AAATTTATAAATTGAGAAGCTATGAAACTTGCTACTAATA

In some embodiments, the intronic domain that promotes trans-splicing primarily in the liver comprises or consists of sequences from the gene AZ12. In some embodiments, the sequences from gene AZ12 comprise or consist of:

(SEQ ID NO: 560)
TATTCATGTGATCTGAAATAAGGCAAAAGAAATGTTAAAGGGATTAGTTGA
CACAGGAGGGAGATTAGACATTTTGGAAGAAAGCAGGCATAGTGAAACACG
GGTTGGAAACGTGGCTGTAGTTAAATTGCCTATGTTTACATCTCAACTCTC
CACTTACTATGTTACTTTGGAAAAAATACTTAAATTCCTCCTACCTCAGTT
TCTTCATCTATAATGTTCTTGAACAGTGACTGATATTAGTAAGTAACCAAA
TTTTATTTCAAGCAAGGGATAGTTAAAAACTCACTTTCTAGTAATAATTAC
TGATCTAAGATACATGAAATTCAATTGATAATCTTTATTTAGAGTGCTATA
TTTAGTTTTTTATTTTAAACTAAAAAATATTTAAAAGGACTTTTTAGGTTC
TCTTGCATGGTCTAAAGTCCCTCTTATTCTGTTTTTCCCAGGAGGTCTTAA
AAGGTTTTAGCCACAGTTTCTCCTTCCATCATGTTTTTCA

In some embodiments, the intronic domain that promotes trans-splicing primarily in the liver comprises or consists of sequences from the gene IP6K2. In some embodiments, the sequences from gene IP6K2 comprise or consist of:

(SEQ ID NO: 561)
GTAAGAATCCATTAAAGAAATTAATTTTAAATGTGCATTACCTGGTTACAT
AAAGGACTCTAGAACAACTTAGGTTCTTTTGGGACTGTTGAAACATTGTCT
TTAAGGACAGAATGTACTGGGTGCCTTTCTCTGTACTCTGAATATGCATGT
GTATTGCGGTGGGGATGGGGGAGTTGGGGGCCCTCACTTGGTCCTTCTCTG
TTGTTTTTTTCCAGGACAAAGCCAAAGGCCCCTTGTTAGCTGGCCATCCCT
GCCCCATTTTTTCCCCTGGTCCTTTCCCCTGTGGCCACAGGGAAGTGTGGC
CTGAATACCCCACCCCGGCTCCTCTGCACCCAGAGCTGGGGGCCACCTCAG
AAGTGTCATCTCTCTCTGAGCACGCATTCCCCTGCAGCAGTCGAGGACTGA
GCAGATTGAGTGATGCTGGGGCAGAGAGGCCTGGGAGGAAAGGTGTTCAGC
CAGTCGTTTGTAAGGCGCTCGTCGGCACCTGCTGAAACGC

In some embodiments, the intronic domain that promotes trans-splicing primarily in the liver comprises or consists of sequences from the gene IP6K2. In some embodiments, the sequences from gene IP6K2 comprise or consist of:

(SEQ ID NO: 562)
AAGGCCCTGGCAGCCATCCCTGGAGCCCTCACTCCCTCCCACTCTGTGCTT
TCCCTCCCTCTCCTCCTTCTCCTCCTCCTGGCCCTCAGCCCAGCATCTCAC
ACCCTCAGTTTTCAATCCCTGGTAAGATTGCTCAATTCTGTTTTGTTGTGT
GGATTTGAGTTTGATTTTGGTAGAAGTGTAGTTGTGTTTTATGTTCAGGTG
TCTCTTGATCACAGTAACCCATAGTCCCCCACTGGGGGGACGGTGGGGGAA
GACTTTGGGAGGATTTTACCCAGAATACTTGCCGCCTGCTTTTTGTCCTCC
AGGAAACCAGAAGCCCGGGTAATTAGGTAGGCCGGGGAGCAGCGTTGAGTC
AGATGGAATGCAGGAGTGGGAGGCCTGTGCTGATTAGCCCAAGCCCTTTGC
TGTTACAGAATGTCCCCAAAATTGCTATATGAAAGCAACCCTTCGATTCAC
ATATGATAATTTTTTATTTCTTTTTATAATGTATTTTATA

In some embodiments, the intronic domain that promotes trans-splicing primarily in the liver comprises or consists of sequences from the gene MST1. In some embodiments, the sequences from gene MST1 comprise or consist of:

(SEQ ID NO: 563)
GTGAGCACTAGTGACGCTTCCCCCATGACCCTGCCTCAGCCCCCACCCAAA
GGCTGGCTCCCTTAACCCCAGTGAACTTTGTCTTTCAGCTGATGACCAGCC
GCCATCAATCCTGGACCCCCCAGGTTAGGAGTTGGGCCAGTTATGGGTCAG
GCCCTTTAGCCCACGACATCCACACAGTCTGGGTTTCATCCAGCCCACCCC
ATCCTACAGACCAGGTGCAGTTTGAGAAGTGTGGCAAGAGGGTGGATCGGC
TGGATCAGCGGCGTTCCAAGCTGCGCGTGGTTGGGGGCCATCCGGGCAACT
CACCCTGGACAGTCAGCTTGCGGAATCGGTGAGGCACAACTGCCTGTCTCC
CACAGAGAGGAGCTGAGGTTGTGTCCTCTGTGGTTATCCCACTGGGGGCTG
GGAATCTATCCCTGCCCCCAGAGGTCCTAGCCAGAAGATGGCAGGTCTAGC
ATCTGTCCCAGGAGTCTGTTACCTGTCCTAATTCCCCACT

In some embodiments, the intronic domain that promotes trans-splicing primarily in the liver comprises or consists of sequences from the gene MST1. In some embodiments, the sequences from gene MST1 comprise or consist of:

(SEQ ID NO: 564)
GGAGGCTGGCGGGCTAGGGCTGAGTGGAGCGCCTGCTTAGAGACCTTCGGG
AGAACTTCTGCCGGAACCCCGACGGCTCAGAGGCGCCCTGGTGCTTCACAC
TGCGGCCCGGCATGCGCGCGGCCTTTTGCTACCAGATCCGGCGTTGTACAG
ACGACGTGCGGCCCCAGGGTGAGGCCCAAGCTTGGGGGCTACAGAGCCGGG
CTGGAAGCCTGGAACCGGAGGGCCGGGGCGAGGTCTCGGCCTGATGGCTGC
CCGCACCCGCCACAGACTGCTACCACGGCGCAGGGGAGCAGTACCGCGGCA
CGGTCAGCAAGACCCGCAAGGGTGTCCAGTGCCAGCGCTGGTCCGCTGAGA
CGCCGCACAAGCCGCAGTGAGTCCCTGGTGCTCCCGGCCCCGCCAGGGCCC
TAACCCTGGGGCGGCATGCTTTGGTGTCTGGGACCAGAGCCTGGAAATGGT
TGAGACTACCCTGCCACGATTTTGCTCCCGCTTCCGCCTA

In some embodiments, the intronic domain that promotes trans-splicing primarily in the liver comprises or consists of sequences from the gene ITIH3. In some embodiments, the sequences from gene ITIH3 comprise or consist of:

(SEQ ID NO: 565)
ACATCCCATTGCCACCAGCCCCATAAAGGGCTTCCCTGGACGTGCCCGATT
TCCAAAGCCTAATCTTTGTAGTCACATCTCCACCTCTATCTGCCAGTGTTG
GGGCCCCTCTGGGGCCCAGCAGCGCTAAGACAGGAGGAAGGCACGGGGCTG
GGGGAGAAGGAACCCCTAGACCCTGCTCACCACTGCCCTTCGGCTTTTCAG
CCACACCGGTGAGCCCCGCCATGTCCTACCTGAGTGAGTACATGCTGGCAG
CTGCCACTCCTCCCACTGCTTAGAGCCTGCCCCTGCCACATGTCCCTCCAC
CGTGGCTACTCACTCAGCTCTCCGGGATGGGCAGATGGACAATGTGCCCTG
AGGAGATTGCGGGGTGGGAGGGCAATGGCATTAGCGAGCGGGAAGGGGAGG
GGAGGGGCATAAGCTGAACTCCTATGGGGGTCGTCGGGCGCCTCCCAGGGC
CACTTGCTTAGCTCAGCCCTCTCTCCCTTTGTATCTGCAG

In some embodiments, the intronic domain that promotes trans-splicing primarily in the liver comprises or consists of sequences from the gene ITIH3. In some embodiments, the sequences from gene ITIH3 comprise or consist of:

(SEQ ID NO: 566)
TGAGTCCCTGGCTGCTCCTCGGGAAGGCTCAGGGGCCTGGGCACGTGCTC
CTGCCTGTCTCGGAGTCGAGCCCATGGAGGCCAGAAGGCTGGGGGAGGCC
CCAGGTATGATGAGAGGCCCCGGGAGTCAGCTCCTTCTCTAATGTGTCAC
CAGTGGACGGGGATCCCCACTTCATCATCCAAATTCCGGAGAAAGACGAT
GCCCTCTGCTTCAACATCGATGAAGCCCCAGGCACAGTGCTGCGCCTTAT
TCAGGATGCAGTCACAGGTGAGGCTTGTGGGCTAGGGCCGGGGCCAGGGG
GCTCTTCCTGGGAGGGCTTGGGTCCCCCGAGGTAGGCACAGGGAACAGGT
TCTCACTCTGCCCAGTAAGGCATCATCTTCACATGCAAAGAAACCCCTGG
GGGTGGGGAGTGCCCAGGGCCACAGGCTTGCTCAGCCCCACATGCCAGTG
CTATTGGGAGATGATTAGCGTGAATGGACCACCTGGGCCTAGGGTAGGA

In some embodiments, the intronic domain that promotes trans-splicing primarily in the liver comprises or consists of sequences from the gene ITIH4. In some embodiments, the sequences from gene ITIH4 comprise or consist of:

(SEQ ID NO: 567)
GTGACAAAGTAGGAGGTGGGGGGAGGGGAGATTGACAGATTGGGCTCAGT
CCTGGCTGTGGAGGGTAGTGCCTTGCCCAGCTCCTCTGGCTGGTGACAGC
CCTCGGGCCTCAGTTCAGTTGTATCTGATCAGGACTTGACCACACAGAGG
CTTCCTTTTCTCCAAGAAGAGGATGGAATAGACAAGGTAGTTTGAGGGTT
TCTGCCAGGGGGAGGCATGACATCCCTGGGGGGGAGCTTGAGGGAGGTCC
ATTATTGTTCATCTCCCAGCTGTTGGGGGAGAGGCTCCCCAAGTTGGCCG
GGTGTGGGGAGAGGGGACATAGCTGCCCTCTCCGCACCCACTCCTGGGTG
GATCTCAAGCCCTGGCTGTGAAGACCCACACCCCACCTGAGAGAAGACTG
CATCTGTCGATTTCTCCAAAGCTGGAGCTGCTGGCTCCCGGATGAATTTC
AGACCTGGGGTTCTCAGCTCCAGGCAACTTGGACTCCCAGGACCTCCTG

In some embodiments, the intronic domain that promotes trans-splicing primarily in the liver comprises or consists of sequences from the gene ITIH4. In some embodiments, the sequences from gene ITIH4 comprise or consist of:

(SEQ ID NO: 568)
TGAGGAAAGTGAAGCCGGGTCTCCTGTCTCCCCAAATTCATTGCACCTAC
GATATAGTCCAGGATTGGGTAGCAAGATTAAACACAAGCGCTTCAAATTG
ATGAAAACCACCCTGACGGTGTCTGTCTGTGTCTCTGTCTGTCTGTCTCT
CTCTCTCTCTTTGTTTTATCCAGAAAGTAGAAACAGGAATGTCCACTCAG
GTAAGCAGCGAACATCCTCAAAGGCTGTGCTGGGTGGTGGTCTGCTAACA
CAGGGTCCAGGGCACCAGCCCTGTGAGGTTGCTGGCCAGGGCCCAGGTCC
ACCCAGGCCTCTCCAGCCCATCCCTGCTGCCAGGAGGCAGTGTAATCTGA
CCCAGCATGCTGGTGCATTTAGTCGCCTGTGCATTCAAATATTGGCTGTG
CCTCAGGCCCTGTCCCCAGCACTAAGAATACAGTCCCCATCCAAGTGGGG
CCACAGGAAGGTGGGTGCAAGATGCAAACCCAGGTCCCACTGATTTCCA

In some embodiments, the intronic domain that promotes trans-splicing primarily in the liver comprises or consists of sequences from the gene IL17RB. In some embodiments, the sequences from gene IL17RB comprise or consist of:

(SEQ ID NO: 569)
TTTGTGTTAGAAGGGGAACATTTTAAAGAACCTGAGGAAAGTACCATGGA
CTGCATTCAGAGCTTTCTTTTGTTTATCTCCTTAGCATAGCTACTCCTTA
ACCTGGGGTAGCCCAGTAAATACTCTAGGCCGTCCTAAAAACCTAGAGTT
AAACATTAGAGTGTGAAGGGGCCTCACATCGGTCCCAAGTCATCCCCTGC
TTGCAGCAGGGAAGGATGACATCCAGAGACAGATGGAGACCCACCTGTGG
TCTTCAAGTGAGGTTATGCACTGGCTTGAGGATCAGGTATACTGATGGCT
AATCCATGGTCCGTCATAGCCTACAGTGGGACAGCTTAGTTCCCCCCTAA
GCTGTGAAGCAGAACCAGTTCATCTTTTCTTTGAAAGTTTGTCACTTGTA
TATGCATTTAAGGGGAAAATGGTGTTCTGCCTAATTTGGCAAAGGAAAGC
TTGTGGTTTGCCGGCTTCAACGTGACGCTTGGTTTTTTCTCTCCCACAG

In some embodiments, the intronic domain that promotes trans-splicing primarily in the liver comprises or consists of sequences from the gene IL17RB. In some embodiments, the sequences from gene IL17RB comprise or consist of:

(SEQ ID NO: 570)
TAGGTGCATTAGAGAATGGGTATTTGGGGCTTTACATTGAAAGCACAGTT
GGACTTTTAGGAAGCCTAATATAGGCAATAGGTCATCGAAGACCAGACAC
AGGGTGACCCATTGCTTTATTGGCTGATGCAGATGAAGTCTCTGTCTGCT
CGAAGCAGAACTCTGGGGCTCTGTGGGAATGAGGCCGTGTTTCAGACAGG
CTAGCCTCTTGGCCAGTCAAGATGGGACTTGCCATACAAGGTGGAAAGTG
ATGCCTGCCACTCTGCAGAGGGAGATCAAGGCCGTGGAAGGAAGGTCACT
TAGAGTTGGGACCGAGAAGCAAATGTTTCATAGGAGAGCGGGTGTTTGAG
ATGGTGGCATTTGAAGAATGGGTAGGCTTCTGCCCAAAAAATGTGGGTAG
GAAGAGTGAGCTGGAGGAGAGATGGCACCAGCAAAGGTTAGGAGGTGGGA
AAGTGGGGGCTGTGTGGGCCAGCTGGCTGGAATCCAGGGCCAAAGCCTG

In some embodiments, the intronic domain that promotes trans-splicing primarily in the liver comprises or consists of sequences from the gene DNASE1L3. In some embodiments, the sequences from gene DNASE1L3 comprise or consist of:

(SEQ ID NO: 571)
GTGAGGACTGCTGTGTCAGACGAATTCCCATCTAGCAGGGGCAGGCTCTG
GTCCCTGAGTGGGATGAGTACCTCTCCAGGGAGGGGGAGGAGGAAGGGCA
TGTGCAGGCTACCACCACTTACAGCATTCTTTAAGGCCTTTCTTCAGCCC
TAAGTATCCCTTTTGCTAAATCTGCCTGGGTACCCTGCATGTGGGCCTTG
GGAATTGCCACACTTTCCCATTAATAACAGATGCCAACATTTACTGAGTG
AATAGCTAGTGCCAGGCACTGTGCTGAGACTTGACATGGACAAGTTTATT
TAATCCTCCCAATAATCCCTTGAGGTAGCTACAGTACCTTTCTCATTCCC
ACTTTGCAATTGAGAAGTGTAAGGCATAGAGCAGTCAGGATTGTTTCAGT
TGCAAGGGACAGAATCTTAACATGGCTCATGAAAAAAAGGAGCAATTGAT
TGGTTTGTGTAACTGAAATGTCCAGGAGTACTTCAGGCATAGCTGGATC

In some embodiments, the intronic domain that promotes trans-splicing primarily in the liver comprises or consists of sequences from the gene DNASE1L3. In some embodiments, the sequences from gene DNASE1L3 comprise or consist of:

(SEQ ID NO: 572)
CTGGAATGTGCCTGCGAGCTGGAATGTCCAGCACAAATATCTTGGTTAAG
GTACAAGGACATAGAATGTACTATGTGCCTGTGAGCATGTCTAACAGCTA
CATAGGATAGAGCTTAACAAAGAGTTATTAGCACAAAGCAAGGAGACTTG
AAGGAAGTTAGTCTTTAAAGGAAACTATTATTTCTAACACTTATGATTTA
TTCTTTAATAAGAAGGGAAACTTTGAAGAGGAAACTTTTTTACTTTCTAC
AACAGTCTACCAGCTTTCTCTTAGCCATGAAGGGCAACTGGGAGAAATGA
GTCTTTCGTGTACTAAAAATAGGCAGCCTAATGACCCCAAGATCATTAAA
ATCTCGGCCACTGGCTGGACCCTCTGGGAACTGGGGACCCAGATAATGCA
GCCTGTGCCTTCTGGAGGGGACAGCTGGTTTCCCTCCAGCAGTGTTTATG
AGACCTTGACAGGGGAAGTGTGTGACCCCCGCCTCTTTCCTGTTTCTCA

In some embodiments, the intronic domain that promotes trans-splicing primarily in the liver comprises or consists of sequences from the gene MYLK. In some embodiments, the sequences from gene MYLK comprise or consist of:

(SEQ ID NO: 573)
GTGAGTGCCCCCAGGAGCTGCCCCTCCAGGCCATGCCCCCATCCCCACCT
TCCTCCTGGCCCCGGCAGCTCCAGCCCAGCAGCCCTAGATTGTTTGTGTC
CTGCAGCTGAGGCTGCCAAAGCCAGAAGGGAGAGCCTGGAAGGGGAATGA
GGGAGGGAGAGCAGGGGAAGGAGGGGGTGATTTTTTTTTTTTTTTTTTTT
GCACACACCCTTATAAGGCTACTGAAGTCATTACCGATGTAATAAACCAA
CTAAGCAAGCGAGCGCCTCTCACCAATGTTCCCATCTATAAACACAGCCT
GGCCGGCCTCCCCCATCCCCTCCCGCTCCCCTCTACCCTCCCACCCTCAG
GGATTTGCTCTCCCCTGAAGTTTCTTTCTTACCCAGCCCCTTTTCCTCCT
GCTACTTTCCTTTTTCCCTACACTGGTTGCAGGGGGAGGGAGCCTGCCAG
CTGCGAGGACCTCTGTGGTGGAGGAGTTGGTGCTGATGGTGGTGGTAGT

In some embodiments, the intronic domain that promotes trans-splicing primarily in the liver comprises or consists of sequences from the gene MYLK. In some embodiments, the sequences from gene MYLK comprise or consist of:

(SEQ ID NO: 574)
TGAGTGGGGGTTAAGGGTGCTTCCATTCCCGAGCAAGAGAGGGAAAGGAA
CTACTTCTGTGTTAAGGGATCATGGAGTCAGAGTCAAGGTCACTGGGCAG
GCCCACCACCAGGGCCTATCTGTCTAGACCAACCTGCTACATCACAGTTC
CCAGCAGGGTTAGCCTTTCTCCTTCCCCTTTTATTGGTTGAGTGAGTGAG
TGATTGATTGAATGATTATATTATATACTTACAGAAAAGTGTTCCTATTT
CTGGATGCCTGCCTTAGCTGTTGTCCAGACATTCTTTTTTTCTTCTTCTT
TTTAAATAAGAGCTGAGAAAATAAAGGGTGACCACACAGTGAGAGGGTCC
TGCCCTAAATCTAAGGCTGGGGTGAATGTCTCAGTCAAGCCGGCGACTTC
CATCTTCCCCGCTGGCCAGCAGCCTGTGATCAAGTCCACACTGAACAGGC
CCTTTGCCCAGAGGGCCTCTTGATCTTTATTGATCTTTGGAATTCTTCA

In some embodiments, the intronic domain that promotes trans-splicing primarily in the liver comprises or consists of sequences from the gene CDV3. In some embodiments, the sequences from gene CDV3 comprise or consist of:

(SEQ ID NO: 575)
TCGCCGCGCCCGGCAGCGTGAGCGCAGAGCCGGCCTCGACCCCGAGCTC
GGAGCCCCGCGGGCCGCGCCCGCCGCCGGCCCCACCCATCCGGGTCGAG
GAGGCCGAGGCCATGGCTGAGACGGAGGAGCGGAGCCTGGACAACTTCT
TTGCCAAGAGGGACAAGAAGAAGAAGAAGGAGCGGAGCAACCGGGCGGC
GAGTGCCGCGGGCGCAGCGGGCAGCGCCGGCGGAAGCAGTGGAGCCGCG
GGTGCGGCGGGCGGCGGGGCGGGCGCGGGGACCCGGCCGGGTGACGGCG
GGACCGCCAGCGCGGGGGCTGCGGGCCCAGGGGCCGCCACCAAGGCTGT
GACGAAGGTGAGGGGCCGGGAGGCCGGCACTCGGGGCCCGGGCCGCGCG
CCGGCGCCCCATGTGCCCGCCCCCGCCTGCCGGGGAAGCGTCCGGGAGG
GGCCGCGGAGGCCGGGCGGACGGGCGCGGGCCGCCACGTGACGCAGGCT
CTCCACCTC

In some embodiments, the intronic domain that promotes trans-splicing primarily in the liver comprises or consists of sequences from the gene CDV3. In some embodiments, the sequences from gene CDV3 comprise or consist of:

(SEQ ID NO: 576)
TCTGGGGCCGCAGTGCCCAGCACAGAGCAGCGTTTATCGGGTGACGTCTA
GGCTGAGTAATTCCGTGTGCTGACCTCAGCCCAGTAGTTTGAGCTGAATT
TTCGCTTTCAGCAGCCGCATTTGTAGCAGCTCTTTTGTGATGACTTAGTC
TCTAAGCCCGTGAAAGAAAAAGAAAACTCTCGGTGGCAAGGTTGAAGTAG
AAATGTAGTTGTGAAATTCCTATTGACTTCTTCCTTCGGCTTTTCTTAGT
TGCTTAAAGTAACAGCTCCCATGCAGGAACCCAGCGGAAGTATAGTGTTA
GCCTACGAGCATGTTTGTCCTCCATTTAGCCTAGTTTGCCCCAGAACGAA
GCAAGGGCTTTGACCAGAAGACAAGTACATACTTAGTTTAGGTTCCAGCC
ACTTGATCCACTTTTCGTAGTGTGTTATTTGCTTAGTTATATGTCTACAA
ATAGCTTTAATTGATCAAATGGCGTTTGTTTTACAGGACGAAGATGAAT

In some embodiments, the intronic domain that promotes trans-splicing primarily in the liver comprises or consists of sequences from the gene PLSCR4. In some embodiments, the sequences from gene PLSCR4 comprise or consist of:

(SEQ ID NO: 577)
GTATGTATAATTTTTGTAGTGCTCAGCAGTATTTTAATCAGATGGAAATT
TGCATTATCCAATATCTAGTTCCTTCTTTATATTGATCATGTTTTAGTGG
CTCAAAGAAAAAAAAACTGCTCTATTACCCTATCTTCAAGAAAGTAATAA
AGTGAATTATTTTGTATAATTTTATCCTTCTATACATCATATAATCATAA
GCTCTTAGATTTGAAAAGGGCTTACATTTTATCTGGTCAATAGCCCTACA
CTTTACCCATTGTTCCTACTCCACATTTACCTTTCAAAGATGAGTAAAAA
AAGGAGCCAAGAGACTAAATGAGTTAGCCAGGGACACCACAGTCAGCTAA
TCACCTCCAGATCAGGAAATAGCATTTAAACCACAGTCAGCTAATCAGTG
GCAGAGCTGGAAATAGAATCCAAATCTGCCAAATCGTAGTGCAGTGCCCT
CGCTTTGGAAGTAAGAAGAAAAGATCTCAGTCAAAATGATGCCTGTTCT

In some embodiments, the intronic domain that promotes trans-splicing primarily in the liver comprises or consists of sequences from the gene PLSCR4. In some embodiments, the sequences from gene PLSCR4 comprise or consist of:

(SEQ ID NO: 578)
TAAAATAATATAAAATGGGGACACTCCATGTGAGGTTCACCCTGGCTTGT
TTGTTCTCCTTAAGCCACTGAGAGACTTCCCTTTTCTATGGCTGGTCCGT
GTTAATCGTTCTGAACTTAACTAAACAACTACAGGCTATGGATGACATGT
AGAGAGACCAGTGAGTAGGGACTTATCTAGGAACAATCTATAAGCACAGC
TTCTTCTGATGTGATTACTACAGCAAGTAGATGTTTTGTTTTTCTGATAT
AGCAGACAACTTTTTTAAAGTGGTACTGAAAGAAAGTGTTTTTCTAATAT
GGACAACTCTGCTAATATCCATCCGATCAGATGCAGATAAGCTAATTTCA
GTTGGTAAAATTTTTTTGATTCTATTGACTTTCAAAAATATATTTTGGTA
TCAATTGATGCATTTTAATATCTATTTCATCAGTTTATTTTACAGTGGTA
TTTTAGTTCTGTTATTTCTGTCTTCTGATTGCTTTGTTTTATCCCAACA

In some embodiments, the intronic domain that promotes trans-splicing primarily in the liver comprises or consists of sequences from the gene BCHE. In some embodiments, the sequences from gene BCHE comprise or consist of:

(SEQ ID NO: 579)
GTGTGTCTGTTTTTAGTGTTTGGTTATTTAATAGATGATGGGTTATGTAA
AAAAGGGATGAAGTATACTCTCCAAGGCACGGTGATGGAGTTACTATATG
TATCTCTGACTTTAAAATAACTTGGTTTAATGAACACATTATTTTTAAGA
CTTTCTTCTATCAAGTTTAGATATGTTTTGAACTCTTACTATATGACCAG
TACTTTAGTAGTTAAATAAAGAAAACTAAAATATAGATTTCACCCTGTGG
AAATATAAAATAGAAAAAGGAAACTAAAATAGAGCCCTACTTGTTATTCA
GATAATCTTTGTGGAATTAATTAATGGACAAAGAAATAAATATATCAAAT
AATCATAATGTATGGAACTAAAAAATAAGATGCATAAATTGGTAAAAACA
GATTTATTGATAAATAATAGAACCTTGATTTAAATGGAGAAAAAAACTCC
GAAGTTTACATATTTAAAAACACAATAAAAAATTTCTAAATATTCTGAA

In some embodiments, the intronic domain that promotes trans-splicing primarily in the liver comprises or consists of sequences from the gene BCHE. In some embodiments, the sequences from gene BCHE comprise or consist of:

(SEQ ID NO: 580)
TTTCCTGGAGTAATGAGATTATTACATAATAAGATTTGTAATTAAACTTG
GTCTTAAACTCAAGCAGTATTAATAAGAGTATATAGAATGAAATAGATGC
AAAAGATGAGTAAGTGATATGATTCCTTATTGTGAAATTTTTGGGCCTAG
GATAAGGGAAGATAATGAAACAAAAGTAACACCACCCTTTGAGCACGTTT
AAGTGTGAATGAAGATATATTTTATTGAAATAAAAAATTTCTTCTTCCAA
TATGTTAAGCAGAAATGCATATAAAAAATTCAACTTTAAATTTCAATTCT
TACAATTTTAAATAACATAGTGGTTCTCAGTGCTCAAACCACTAAGCCCA
GTTCACATACGTTTCATATCATCAATATATATCCACATTTTTTCTGTCTT
AAATTCTTTAAAGCTCTTGTGAACAGTGTTAGAAAACAATATTCTTTTTA
ATCTAACAATGATTTCAATTTTACTATAATGTCTCTATTTTATTATTTA

In some embodiments, the intronic domain that promotes trans-splicing primarily in the liver comprises or consists of sequences from the gene MASP1. In some embodiments, the sequences from gene MASP1 comprise or consist of:

(SEQ ID NO: 581)
GTGAAGTTCACTTTATAACCTACAGACAGTGACACCTCAGCTCTGCACTGG
GCCTTCCTGCTCCGTATTGTTCCCTTAGGGATTTTGAGGCTTGGGTGGGGG
TTGGTGAGTGGGAAACTAGGAGCCCAGACCCACCTCCAAAGTTCAATGGGA
CTTTGAACCCCAGAAAGCTTGATTTGTTTGTTCTACATGTCATGGACTCTA
GCCTCTAAACACGGTTAAGTTTTTCTGGTAGCTTATTAAAGGCTTAGCTTA
TGAAAATTCTCCCAGAAAACAGCAAGTTTAGACACCCCCATGGTGACAATC
CAGCTGTGCACCATGAGAACTGTACACCAAATTGGCCAGAATGAGCCAGGC
TCTTGAGAACCACAGTCCTTGCAGTAATGAACTTGGCAGTTACACTTAGAA
GTTCATATTCCTATCAGTTCAAGGAAAAAAAAAAAAAACACTTAACTAAAA
GTAAGGAGCCATAGTTTCAAGTCTTGGCTCCCACTAACTT

In some embodiments, the intronic domain that promotes trans-splicing primarily in the liver comprises or consists of sequences from the gene MASP1. In some embodiments, the sequences from gene MASP1 comprise or consist of:

(SEQ ID NO: 582)
AAGGGGTTTTAGAGAATACCAAGTCCAGCCTCCTCATTTTATGGATAGGAA
AACAGAGGCTTAGAAGGGGAGCAGAGAGGAGGGCTGGCTAAAGGCACCATA
GCACATAGTGGACTAGGCTGGGCTTTTTATTTATTCGCTGTTCTTGATTGA
GCACCTACTATGTGACAGCATTGCTATAGGCACTGTGATATAGCAGTGACC
AAGAATGAGATTCTGCTCTCATGGACCCTAGGTTTCAGACTAGTTGAGGGT
GGGACAGAATAGAAAACTAAACAAGTAAGCCAGTAACACATCATAGAAACC
AGTGCCCCTAGCTCTGCTGCAGGTAATCTGTAGCCCAGCTGCCTTTCACGT
CTTCCTCCCCACCACACCACCTTTCTCTGAGAAGAACAATCCAGTCTTTCC
TCCACAGAAGAGGATGATGGAGCTGCAAGCTGCACCCCTCCAAACACCCCA
CAGATAGCTTCTCTGTTCTCACAGTATCTTCTCTCCTCCA

In some embodiments, the intronic domain that promotes trans-splicing primarily in the liver comprises or consists of sequences from the gene IL1RAP. In some embodiments, the sequences from gene IL1RAP comprise or consist of:

(SEQ ID NO: 583)
AACCCAAAAGGACCTGGCCCCAGAAGCTTCTCTCTAAACTGATACAGCATC
CTGGCTCAGAAGTTTAAGCAAAAGAGACATTTTGTTTTGAAGAGAGATCTA
TAAAAAGAAGCCAGTATTGGGATTTGGTCTAGACAATGGGATAGTGTGTAG
CGTAGATGGTATAGTATAGTGTTAGAATCTTACCATTAATACTTTGTAATC
ACTGCATTATAGGTGGCTCAGCTTATCAACCTTCCTCGCCCTCACCTGTAG
TGGGGGTCTGTGGTTGCTTACTTGTCTGATTCCTACACAGTCCATAGCTCT
TGAGTCACTCTCAAATGCTTTGATTTTCTTCTCTCTATTTCTAGTATAAGT
CATAGTAGAACAGAAGATGAAACAAGAACTCAGATTTTGAGCATCAAGAAA
GTTACCTCTGAGGATCTCAAGCGCAGCTATGTCTGTCATGCTAGAAGTGCC
AAAGGCGAAGTTGCCAAAGCAGCCAAGGTGAAGCAGAAAG

In some embodiments, the intronic domain that promotes trans-splicing primarily in the liver comprises or consists of sequences from the gene IL1RAP. In some embodiments, the sequences from gene IL1RAP comprise or consist of:

(SEQ ID NO: 584)
TCAGACTGTGTCTAAAACTTTCTTTGTGTCTCTCACCTAACACAGAATCCC
AGAAAATCCCTTCAAGGATGTTTTCAATGGTCTTGAGAAAAGTAATGATGA
GTTATCACACATGAAATCATGAAATCAGGTCATTCAGAATCCTCCTGGCTC
CTAGTCTATAGAGTGTGTAATATTTCTTCAAAAGTGAATAACATAGCACAT
AATTGTTACATTTTACAGAGACTTAATGCTTTTTTAAAGCTGATGAAAAAC
AGGATTCACCTCAAACTTTCATTACAACTTTAAAACTAAATAGCAAAGGCC
TGAAATGTATATGCTGCGTATGAAGTACTAGTGAATCAGTTCAGGGTAATT
CATTGCTTCTGCTCCTCAGAAGCCAGTGTGAAAAGGAAAGACTGAAGTACA
TGATGTAAGGCAGTTTCCCAAATTGAGTTCTCGGGAACACTACTAAGAATC
AGTATTGTCCTGCAATGCATTATTGTACTCTAGAATCAGA

In some embodiments, the intronic domain that promotes trans-splicing primarily in the liver comprises or consists of sequences from the gene PI4K2B. In some embodiments, the sequences from gene PI4K2B comprise or consist of:

(SEQ ID NO: 585)
ATTTAGAATTCAAAGAAAGAGTCTTAACAACTAAAAAAAAAAAATGAAAAA
AAATTATTTATTGTATTTTATGTCATAAAAAGAATCTGGAAAAGTTCAGAT
AAAATTGCACTGTTACTGAATAAGCAGGATAGGTTTAAAATTTGGCCTCCA
TAATTAAATTCACCTTAATGAATATTTTTAGAAAAACGGCATTCTTTTTCA
GAGTGTCACCTTGAAGACATGTGTTTATTCTTTTTTTAAATTTTCCCAAAA
TAGCTGCTATGCTCCTTGAAAGTCTAGGGGTAAGCATTTTTTATTTGCTAT
GTATTCTTTATGTGATTATTTAAAATTAGTGTATAAAAATGGTTTTCTTGA
TAAATGTGGCTTATCTAAATTAGTGTTAAGTTTTCTTATTGTGTTTACATG
ACATATTTTTTGACAGTACTGCTGAGAAATATAAATATTAATCCCCTTGTT
CTTGTACTTTCTTTTCTAACTATAGTTCTTAAAATTATAG

In some embodiments, the intronic domain that promotes trans-splicing primarily in the liver comprises or consists of sequences from the gene PI4K2B. In some embodiments, the sequences from gene PI4K2B comprise or consist of:

(SEQ ID NO: 586)
TATTGAAGTTCTCTCATTTGTTCACGTAAATCTTGTTCATTTTCAAACTAT
ATTCTTGTTATCCAAATCAAAGCGGTAAACTTTTGTTGCAGAAGTCTTGAA
GCCACTTTCTCTGTCTGTCTTGGCAAACAGATGTAGTATTGACTCTCCTGG
GCAGAATTCATCCTGTGTGGGATAGATTCTGACTCATAGCAGGCTCCTAGA
TCAGTTCCTGTCCCCTAGGGAGGAATTTCCTAGGGGCAGGGCAAAGTCAGA
TATTGGAAATGTTATCTGCATAATGTGAGTAATTAAGGCTTCATTGTATAA
ATTATGTTAGGTGCTTATTTTGACATTATGTAACTCTTAGTGTAGTCTATA
AATACACTAAACTCTTAATTTTTCCTGTGCTATAAAGGAGCAATGACGTAG
ACCAGGGATGTCCAGTCTTTTGGCTTCCCTGGGCCACACTGGAAGAAGAAT
TGTCTTGGGCCACACATAAAAACAGTAACACTAATGACAG

In some embodiments, the intronic domain that promotes trans-splicing primarily in the liver comprises or consists of sequences from the gene SULT1B1. In some embodiments, the sequences from gene SULT1B1 comprise or consist of:

(SEQ ID NO: 587)
GTATACTCCAGACTCCATGTCAGTTTTTTAGTATGGCCCTATCCCTTTACC
CACTCACATAGTTTATTTTTTAAAATATTTTTTGTTTCTTTAACCATATAC
TTGCAACAATCTATACTATACCCACTCACGTTGTACGTAAATCTTCAAAAG
CCATTAAGTTTTAGTCTGAGTTTATATACTCAAGTTTTCCAAACTTGAGCA
GCAACAGAATCCAAACCTCAGCACAATATTACTACTTTTTTTTCCAACTCT
CCCTAGATCTTCAGGCCTTCTGTAAAAACTGAACTTAAGGACGTTAGTCCT
TATTTGAAGATTTGGTATAATTCTTTGTTGTAACATTTGTTCCTGCTTATC
TGCTTTTGGGGAAATTTTATACTTCAAATTCTATTTATTTAATAGCTACAG
TGCTATGTAGATTATGTGTTTTTTTCTTATCTGACTTTTGACAATTTGTGA
TGTGTGAGAAACTTTTCTAAGTTGTTGACTTTATGAATGA

In some embodiments, the intronic domain that promotes trans-splicing primarily in the liver comprises or consists of sequences from the gene SULT1B1. In some embodiments, the sequences from gene SULT1B1 comprise or consist of:

(SEQ ID NO: 588)
TAATGAAAATATAGATAAAACAGCTGATATTATATACAAAGTTAACATCAT
CTATGCTACAGTACCTAATACAACACGATAGCCAGTTTTAGACTTACTAGT
TATTGATACCTCTTTGGCATGAAGAAAACATTTAAATAGTCATTTATAGCA
ATGCACAGGTATAATAAAACTCCAAATTATTAAAGAGGGTATTATTATCTC
TCCTTTTGGAGATGGAGAAAGTGAAGTACAGAGATTCTGAGATACTTTCAA
AGTGTCTCACAATGCTTAAGTGATAGAGTTAGCATTTGAACTCAGGGTGGC
TAACCTCAGAATGTGTATCCTTGAATGTTACACAATACTGACTCTAGTAAT
GTGGTGAAATGCCTGTGATTGTTTCAGAATTTATCTGCATTACATAATGCT
CAAAAATGTCTTGAAAAACTGGTTGGCAAAATAAGGAAACGTAGCCTTTCT
GGAGACTCTAATATTTCCCTATACTTAACTAAATTTCTCA

In some embodiments, the intronic domain that promotes trans-splicing primarily in the liver comprises or consists of sequences from the gene CXCL2. In some embodiments, the sequences from gene CXCL2 comprise or consist of:

(SEQ ID NO: 589)
GTAAGTCTCGCCCATCGCTGCCGCTGCCACTGCTGGGGTCCCCGACTCTCC
CGCTGCCCCAAACCCTGTCCTCAGCCCGACCTCCTGTCTCACGAGATTCCC
TTCTCTCTGCAGAGCCACACTCAAGAATGGGCAGAAAGCTTGTCTCAACCC
CGCATCGCCCATGGTTAAGAAAATCATCGAAAAGATGCTGAAAAAGTAAGT
TATAATTTCCATGTACACAGGCGACTGGAGCTGTTGGTCAGAAATACTGGC
GTCTGCCCCCTAAAAAGTAAATCAGGAAAACCCAGGGTTAGCTGCAGGACT
GAAAAAATTATTATTTTCACAAAGTTGCCATTAAGGTTATTAATCTGTTCT
GGTGCCAGAGGATATTCCCAGTGCCCAGGGTCACCACCCAGGTTCTTCCCT
CCTGCCAGTGAATGTAGGTAAAACTGCTTTCATGTAAGGTCTCATTGGAGT
TTGCCAGTTGTGCTGAAGTGTTTTCCACCTGTTAAAAAAC

In some embodiments, the intronic domain that promotes trans-splicing primarily in the liver comprises or consists of sequences from the gene CXCL2. In some embodiments, the sequences from gene CXCL2 comprise or consist of:

(SEQ ID NO: 590)
ATTCGGGGCAGAAAGAGAACATCCCACAGTTGGCGGGAGTTACGCAAGACA
GTCAGACCCGGACGTCACTCGTGAGTGCCCCGACCCCCCTCCACCCCAGAG
GCGGGGCCATCGCCTTCCTTCCGAACTCGGGATCGATCTGGAGCTCCGGGA
ATTTCCCTGGCCCGGGACTCCGGGCTTTCCAGCCCCAACCATGCATAAAAG
GGGTTCGCCGTTCTCGGAGAGCCACAGAGCCCGGGCCACAGGCAGCTCCTT
GCCAGCTCTCCTCCTCGCACAGCCGCTCGAACCGCCTGCTGAGCCCCATGG
CCCGCGCCACGCTCTCCGCCGCCCCCAGCAATCCCCGGCTCCTGCGGGTGG
CGCTGCTGCTCCTGCTCCTGGTGGCCGCCAGCCGGCGCGCAGCAGGTGGGT
CCCGGCGCCCTGGGGTCCCCGGGCCGGACGCGGCTGGGGTGGGCGCCCCGC
GCCGACAGCCCCGCTCAATCAGCGAGTCTCTTCTTCCCTA

In some embodiments, the intronic domain that promotes trans-splicing primarily in the liver comprises or consists of sequences from the gene HSD17B13. In some embodiments, the sequences from gene HSD17B13 comprise or consist of:

(SEQ ID NO: 591)
GTAAGTACAGCACAGAACACCCAAATACTAAAACACCAATAGAGCTTTTTT
TTTTGCTTTTTTTTTTTTTAGACAGAGTCTCACTCTGTCACCCTGGCTGGA
TTGCGGTGGTTGCAGTGGCATGATCTTGGCTCACTGCAACCTCCGCCTCCT
GGGTTCAAGCAATTCTCATGCCTCAGACCCCCAAGTAACTGGGATTATAGG
TGTGTGCTGCCACACTACACCCAGCTAATTTTTGTATTTTTTGATAGAGAC
AGGTTTCCCCATGTTGGCCAGGCTGGACTCGAACTCCTGACCTCAAGTTAT
CCTCCTGTCTCGGCCTCCCAAAGTGCTGGGATTACAGTCATGAGCCACCAT
GCCTGGCCCAATAGAGCTATTATTATGGAGCATCTTTCAGTTGTGAAAATT
GGCATGGAAACTCTCCATCCCTGGGGAGAACAGTTATTTCCTCTGTTATTT
TCCTACCCAGTCTATAAAAAGAGAGTGATTCATTTTCTCT

In some embodiments, the intronic domain that promotes trans-splicing primarily in the liver comprises or consists of sequences from the gene HSD17B13. In some embodiments, the sequences from gene HSD17B13 comprise or consist of:

(SEQ ID NO: 592)
TTTACCCAGACCCTATTCAAGATGGAGTTGCTCTGATTTAAACGCCTCTGA
CAAAATGACGACCTCAAAACAATCCAGCTTTATGGAATACCTCCACAAGAA
AGAAAGTATACTTAGCTATAGAATTTTCTCCTTGCATCCAACAGGCTTTGA
GATGTCAGATGTTTCCTTCCTGTCCCATGATTAATCCTAGCCATTCCTCTT
TCTTGTCTGGCTCCACTACTCCTTACCATCTAATGCCTCGCCACCATTTTG
ATATTTTGACTAAGTGAGCTATGAAACACACCTACTGGATATGAAAGTATA
AGTTTCTGATAACAAAACATCAACATGGGATGTGGAGGAAGTGGGTAGGGT
GGCATTAATGCAGCAAATCCTGGAATATTTTAAATCTTCATTCTAAATTTA
GTAAAAATATAGGATAATTTTCCTGCCATCATTTACTTATAAAATTAAAAT
TTTAGAAAATAAAAATAATATTTTCCTCTTTTTAATCACA

In some embodiments, the intronic domain that promotes trans-splicing primarily in the liver comprises or consists of sequences from the gene ADH4. In some embodiments, the sequences from gene ADH4 comprise or consist of:

(SEQ ID NO: 593)
GTCAGTTTTTTTTTTCTTCATAGCTTTAAATTCTTTTCCTAGTAGTTCTGA
AATTCTTAGAGTGAAGGTTATTGAAAAGAAATAAAAATAATTTATTCAACC
ATTGAATCCCTAGAGCATATCCAAATACTACATAAAAACTATTAATGACTT
ATAAATAATTTATATAAAAAGGTAATTATAATTTGCAAATTGAAATTTAAT
TTGCATTTGAATGAACCAACATGGAGGAACAAAAAATAAACATTGACCTTA
TTGAGATAATTTCTTATATAGAGTGGAAAATAATCTTAAATTTTTCTTATA
AAGAAAAATTTTAAAATAGATATAAGTACAGAAAATTAAAGATGGAAGGAG
TTTAATCTATTTGAAGCAAACCAAATCTGACCTAATAATTTCATTCATTCA
GCAAATGCTGATTAAACTACTTCGTGTCAAGTGCCATGTCTTTATTTTGCA
TTTTGTCCACATTACAGGTTGGAAAAGTGTAGATTCTATC

In some embodiments, the intronic domain that promotes trans-splicing primarily in the liver comprises or consists of sequences from the gene ADH4. In some embodiments, the sequences from gene ADH4 comprise or consist of:

(SEQ ID NO: 594)
TTTTTTTTTTTTTGAGACAGAGTCTCACTCTGTGACCCAGGCTGCAGTGCA
GTGGTGTGGTCTCGGCTCACTGCAACCTCCTCCTCCTCCCGGGTTCAGGCA
GTTCTCCAGTCTCAGCTTCCCGAGTAGCTGGGACTACAGGCAGGCACAACC
ACACCCAGCTAATTTTTGTATTTTTAGTAGAGACGGGGTTTTGCCATGTTG
GCCAGGCTGGTCTCGAACTCCTGGCCTCAAGTGATCCACCCACCTTGGCCA
CCCAAAGTTCTAGGATTACAGGAATGAGTCACCTGCACCCGGACTGTGCTG
AACATTCTATTTGTAATGGAACACTGGGCCCTCAAGGCTCAATCTACAATG
ATACTATGATGTCATTAGCTTGAATCCCATTATCTTACATGCAGAGTTGGA
TTAATTTTTTTCTAAAGCATTGTACATTGCCACACTGGAGTTCATATTCTA
CTTTTCCACTGACTCAAGTATTCTTTTATGCTCCATCACA

In some embodiments, the intronic domain that promotes trans-splicing primarily in the liver comprises or consists of sequences from the gene ADH6. In some embodiments, the sequences from gene ADH6 comprise or consist of:

(SEQ ID NO: 595)
TTTGAATGTCTTGTTATGGTGTAGCTCGTTGCAAAATTCACCTAATTTTAT
CACAAATGCAGGAACTTTTTTTCCTTCCTGTTAATATACAGTCAAACATAT
TACCAAGTACTGGAGACTATCTATATCACTGTATAACCATTCACTGTTGTT
GCCAGCTTCAAGACCACATTTGAATCCTATCAGGCCCAGTAAACTCTGTGA
ATAGACTCTGTGAACTGTCTCTAAGTCTCACTGTTTTGTCCACATTAAGTG
TCTTGTTTGAAGCACAAAGTAAAATCTTTCTTTCCAATCCCTGTCACTGCA
CCTCCCTAGACAGTCACTACATGGGGCAGTTTATGGACTTCAGCAATGTTT
ACCTAACTCCAGATCCACACTATAGGAGTCCAGCCTTCAGGGAGAACTAGT
TTTTTACATTTTTTGCCTGCATAATGAGAAGCTTTAAGTTTTTCATATTTT
TACCTGAGAACAAGAATAATTACATTCTAGGCTATCACCC

In some embodiments, the intronic domain that promotes trans-splicing primarily in the liver comprises or consists of sequences from the gene ADH6. In some embodiments, the sequences from gene ADH6 comprise or consist of:

(SEQ ID NO: 596)
AAAATACATAATCCTGAAAGTATTTAAGTGTTCACCTACCGTTACTTTTGC
CATTAGCATTGTATTTCCAATATGGATTTTTTTTTTACATGACATATTAAT
GGAAGTCAGCTTATAAAATTGTAGCTCAAGTTTAAAAACATAAGAAGGTAG
TTCACAAATAGGCCATTGTTGTATTTGTTGTTCTACTTTGGGGGAGAAGAG
GAATTATTCATAGGATTCCTAATTCTGAATCCTATCTTTGACCCAAAAAAC
AGAAACCAGCCATAACCCAGAAACTGGCATTCCCCACACGAGTATAAGCTT
GCTTACCTACTTTTTACCCTTGGAGTTTCATGTCATTTTAAAAAATCTTCC
CATGGCTTATTTAATCTTCTCATTTTTCACCTAAAGAGAAAAAAAATCTAG
TTTACTGCTCTACAAAGTGAGAATTTGAAATCCAAAACACAAATGTTGCTC
TAAAAATGTTCCAACCTTTTTTCTCTTCTTCTTTTTTTCA

In some embodiments, the intronic domain that promotes trans-splicing primarily in the liver comprises or consists of sequences from the gene ADH1A. In some embodiments, the sequences from gene ADH1A comprise or consist of:

(SEQ ID NO: 597)
TACTTGTGTCATCTTTGCCATTGAGAAAAGCTATTTCCTATGACTTCCTGC
ATTTTTGGTATCTTCATAATCTTTAGTCATGGAATCCCAGTGGAGGGGACC
CTTCACTTGCCCTGAATATACACATGCTGGGCTATTGTGCTTGTAGTCTCC
TGCATCTATCTCAATTTTCACTGTCGGCATTTTCGCCTTTTTTTTTTTTTA
ATAAAATATACCGAATTCCTAGGGTAAAAGCTGTAGTAGGGTAAAGGATAT
ACTCATATTTACAAGTGGTGAAAGTCCAAGAGTTGTAAATCCAGGAAATTT
CTTAGGAACTCAAATAAAATGTCACATGTTTTCTTACAGTGAAGGAGAATG
TTTTTCTGACATTTATACTATGTCTTTATGGTCAATATACAATTGATTTTT
AAAAATAATAGCTGATTTCATGCTTCATATGACTAAGGTACAATTACTAAT
TGCAAAAACTGAACTATTCCTGGAATTATGTTCAAAAAAT

In some embodiments, the intronic domain that promotes trans-splicing primarily in the liver comprises or consists of sequences from the gene ADH1A. In some embodiments, the sequences from gene ADH1A comprise or consist of:

(SEQ ID NO: 598)
ACTCTATACTAGATATGCGACCAGACCAAGTTACAAATCTTTCTAAGTCTC
AGTGTTTATAATCTCTAAAATCAAAATAATATGAAAATCTACCTCCTAGTG
TTGATGTTTAGGTTGAAGTTTAAGACAGAAAATGCATGAAGAATGTGACAA
ATAGCAAAAACTTATCACAAGTTATTATCCATTGTTGTTTTATATTCCTTA
TTATAACAGGAACGTGGGCACACAACAATGATTTTTTGAAGATGCTAACGT
AGTATTGAAGTCTAAATGACAGGCATACTCAGAATGTTGTGAGTGAGCTTA
AAGTTGAAGAAAACACAAGAGAAAACCAAAAAAGATGAGCTTTCCTTTGAC
TTCACTTGACCCGAATGCATGGGGGTGGGATTAGAGTGGCTGAGAACAGAC
AGATTTTCACTAAGTCATTTCAAATGCTCCTTGGACTCTCACATGGAAGCA
TGTCTAAATGCTAATACAATGTCTCTTCTTTCCTATTGCA

In some embodiments, the intronic domain that promotes trans-splicing primarily in the liver comprises or consists of sequences from the gene NDUFC1. In some embodiments, the sequences from gene NDUFC1 comprise or consist of:

(SEQ ID NO: 599)
GTAAGTGTTAAGAGATCTTTTCAACTGTAAAATATAATATGTCACCTTGGA
TGAGTAGCTGTCTTCTTTAAGCTTCATCTGTAAATTGAGGCTTATAACCAC
GGCATGATATGTGCTCGTTTTGAAGATGAAATAAGACAATGAATGTAAACC
ATATGATACATTAATGCACCGTGCCTGGACCACAGAATTCAATCCAAATGC
TAGTTATTATTCAGCACGACACTTTTCTTTCTTCTGGCTCATGTAGGAAAA
TTTTAATAAACCTCTTGATGAATAGCCGTAGAGATGGGGTGAAAAACCCCC
AGAGGACAGAGAATAGGTAAACTCTTTTTTCTTTTAATTATTTTCTTCCTA
ATTCCACTGTATAGACAGAAGGCTGAGGCCGGGGTGCTAGAGAACCTTGCC
GTGCTGGAATTCACGTTGACGCCCCCACGGAGCTCTGCTGCAGAGCCCTCG
AGTCCCGCACTTCCGGCCGCCAGGTGGCGCTGGTTCTGTT

In some embodiments, the intronic domain that promotes trans-splicing primarily in the liver comprises or consists of sequences from the gene NDUFC1. In some embodiments, the sequences from gene NDUFC1 comprise or consist of:

(SEQ ID NO: 600)
TTTTGGTTTTAAAATTAATGACATAAAGTTTAAAGGATTAGTTTTAAAGGG
TCAAAATGTATTGTCTGTTACCTATAAATATTTTTACTGGTCTTTAGACAT
CAGATTGAACTGAATTGAATGTACAAAGATCTGGCTCAACTGTGCAAGATA
AAACCTATTCCTCAGAACGATGTCTTTAATAGGCAAAACCAAATAAATTGT
TTACTACTACCAGACACTTGTTTAAGCTTCTCAGCAACCCTATAGGTTTTA
TGTTACAGATGTAAAACTTAGGTATAGAGAGGTTAAGTAATTTGCCTAGAA
ATCATAGAGCTAAAAAGTGGTGAGCAGAATGGCTTTGAACGCTGATTCTAC
AGCTTATGGTCTAGGGTGGTCCAAGTCTGTTAGGTGGGCTAATGAGGATCA
CTAGATCTGGAGTCAGAAGACCTCAAACTGTATCCCACCTCTGCCATCTGT
GTGATCTTAAGCCTACCTAAGGCACTAATTCTACTTCACA

In some embodiments, the intronic domain that promotes trans-splicing primarily in the liver comprises or consists of sequences from the gene SORBS2. In some embodiments, the sequences from gene SORBS2 comprise or consist of:

(SEQ ID NO: 601)
GTATAATATCACTAACTCACACAATGTCGCACTTGAATGGCTTCTAGCTCT
TCTGACTGTTTTTGTTCTAAACGTAAAAAGCAAAGGAAAATGAGATCTTGT
AGGAAACATTTTAAATGTGTAATTAAAAACAGCAGTCACTTGCCAAAAATA
GGTTGAGTAACTGCGAGTGTTAATAACTATTCACGTTTTTGTTATCTCATG
AACTTAAGTAGCATAATATTTCATTAAGATAACTCAGAGGTTTTGGGGTTT
TTTTTTTTAACCTAAAAACTTTTGGGGTAGGGGTACAGGGGTACAGGATGA
AAAAAGGGATTTGTAGGAAACTGAATCTTCATTAACTGGATTCTTTGTCAA
AGGTTGTGAACAGAATGATAGAACATTAGTTTTGAAAGAGACCTTTGCTTT
CCTTTAATCCAATCAGCAAATACCCTTCTACTATATATATTTAAACATACC
AAAATCTTGACCAATTAAGATTCACATGTACCCACAGATA

In some embodiments, the intronic domain that promotes trans-splicing primarily in the liver comprises or consists of sequences from the gene SORBS2. In some embodiments, the sequences from gene SORBS2 comprise or consist of:

(SEQ ID NO: 602)
TTTACTGCAAGAATTATTCAAGCTCTCTTTGGGAATTATAACAGCTAGAAG
ATGGTAAAACTTAAATGATATTAACTTAAAAGTATGTTGTACCATAGCCTT
TAATCCTGACATGGAGCTAATCCTTGACTCTAAAAGCCAGATCCAGTGCTT
ATAGATTTAGTATAAATTGAATTTAAATGGTATAAACAAAGTTGAGTTCTG
TGCCTGAGTTTTCACAAAGAAAACCATAAAATGTTAAGTATTAAGTCTTCC
ATGCTTTCACTAAAACAGTGATACCTATTTCTAAGACAGATCCCTGGTCTT
CTTTTGTTTTGTTTCGTTTTGTTTGTTTTAGTTTGAGGGTGGTGTGAAATT
GGCAGGTGCATTCCATGATTACTAATTCTAACTGTTCATCTTTCTCATGGA
TGTCTGGCACATTTCTCTTAAAACGCATGATATTGCACGTGCTTTTCCCGT
TGCCTTGGCCTCTGCTATATCATTTTTTTGCTTCCAGATA

In some embodiments, the intronic domain that promotes trans-splicing primarily in the liver comprises or consists of sequences from the gene KLKB1. In some embodiments, the sequences from gene KLKB1 comprise or consist of:

(SEQ ID NO: 603)
GATATGTACACAACTAAAACAAAACTAAGATATAAGATCATATTAATGTAA
TGATAATAAAAGTGGATTGTCTCCTGTTCTAATTTTAATAGGCACAAGGCA
TTTTTGTTAATCACTTCTTATTAAAGAATTTTTTATAATATTCAGGAAAAT
ACAACAAAAAAACCCTTACATTCCTAAGGCCTCAGAGTCAGAGTAATATAA
AGGAAATGTAAACACATGCTGAGTAATGCAAGCATTTGGCAATGGTGGTGA
CTGGAGCTGGGAGCACAGCTTTTATTTCTCTGAAATAGGAATTTGCCCCTT
AGAGTTAGACCAATTTTGCCTTCCTCTAAATGGCAAACAGTTTGGAGATAC
TTTAAAGGACATTTTTTCACTTAGGATGTTTAGTACTATGAATAATAAATA
GTCACAATTTCCTTAACTATGGTGACAAAATACAAGCAAATTTAGCCTCAT
GTCATTTCCTAAGGAACATCTTCTCTCTGTGAGTTCACAG

In some embodiments, the intronic domain that promotes trans-splicing primarily in the liver comprises or consists of sequences from the gene KLKB1. In some embodiments, the sequences from gene KLKB1 comprise or consist of:

(SEQ ID NO: 604)
CGAGTATGCATGGAAAATCGCATCACAAAGGCGAGTATGCATGGGGAGCAC
TTGCTGCTGTACTTTCATCACTTTTATAGTCTGAGTTCTTAAAAGTTTCGT
TCATTTCCCTCAAAACACTTGAACCTGCAGTTTCAGTAGGTACTGTTCTGC
CAGGTGCAGATTAGTTAAGAGATTAGCAGACTTCTCTGCCTATCTTCTCTT
ACTTTAAAACAAATGTTACCATTGAATCAAGGAAGCAATAGCCATGAGAAA
AAAGAAGGATCTGACGCCTTTGAATGAAGATTCAAAACATGATCTTCATGT
TTTGTATTAGCTTGGAGTAAAATCCACTTGCTGGCAATATAGCCCTTAAGC
TTGTTGCCTCTTCTCTTTGTTTCAGAAACTAGAGCCCTGTTTATTCTGATC
AAGGCTCTGGCCCACTGTCTTTATCTCAGATAACCCACCCTCTTCTGCACA
CAGCATGGAGCTAAGAGAAGGGTGTCTAGTTATGTAATAT

In some embodiments, the intronic domain that promotes trans-splicing primarily in the liver comprises or consists of sequences from the gene C6. In some embodiments, the sequences from gene C6 comprise or consist of:

(SEQ ID NO: 605)
GTAAGAGATACCCTACAGACTGTGTCTGGAAATTGGGAAAACCAGTCTAGT
GTAACCAATTTGATGGAAGAGCTGAGCTTTTTGAAGCACAGACATGAAATA
AATGAATTTTCCACATAACTGATATTCCTGTGCTCTGCTGAATTCCTCCTT
CCCCATTGGGTAGCAAGTTTCTTAATTATCTCTCAGCCCAGTTCCCTGTTT
AAAATGAGATTGTTCATTATAACAGTCTCATTTTGGTTTTAAAGACTTAAC
TTCTGCACATGTAAAAACTTTAAACTCAGTTATATCAACACTTTCCTGTTC
CTCTCACTCAAGCCCAGGGCTTGGAGTGGGGTGGGGAAAACAGGCAAAAGA
AAACTGGTTGGTGAGTGAACACACTACATTGGCTACGTGCCCCCTGTTTTT
TTCTGATCTTCATATAGAAATGCTTGCTTTCCTCTCTTGGGCAGCCATCAT
TCAGAAGATCTCTGTGTGTTTGACACAGACTCCAACGATT

In some embodiments, the intronic domain that promotes trans-splicing primarily in the liver comprises or consists of sequences from the gene C6. In some embodiments, the sequences from gene C6 comprise or consist of:

(SEQ ID NO: 606)
AATCTTTCTAGCTACATCAATTTTTTATTATATGCTTCTCTTTGATAGCCC
TAAGAAAGGCAACAATTCTATATTTATATTAGTGTGCTCATTTGATTAATG
TCTGTCTGCACCCACAAGACTATAAGCTTGGTGAGCACAGGATCTGGGTCA
GTTTATGCTCAATCTACTCTCTCCAGAACCTTACAGAGTGATGAGAATATC
ATGTATACTCAATAAATATTTGAAAAACAAATGAACAAAAATTTCAACAGT
TCCATTTTCTTTAGTTATCTGAACATAATCGCAATTTTAGGTTGGAAAATG
ACACCTCATGTGCACTGGAAAGGACCTTTCAGGTGGAATATTTAATGACAA
TGTATTTAGAGAAGCCAAAATGAATGATCTAACTCCAAGAGATAAATATGA
ATCCTTTACCACTGCCTCTTCTCTGAATTCAAGCTTATCACTTAGAATCCA
TGCTGTGCACTGTTCTTTTCTCCTTTTTTCTTTTGTTTCA

In some embodiments, the intronic domain that promotes trans-splicing primarily in the liver comprises or consists of sequences from the gene MRPS27. In some embodiments, the sequences from gene MRPS27 comprise or consist of:

(SEQ ID NO: 607)
GCAAGTGCCCATTCCAGAGACTCACTCACCTGACATATTACTACTGGAAGT
TGAAAAGTCCCCACATTTATTTGTCTGAACTGCTTAAAAAGTCACCAACAG
GAATTCTTCTCAAACTTGCCATCCAAAATTGAACCCCGCTGGCCCAGTGTT
TTGACTTGTTTATATTCCAGATAGCCACTCACGTAAGTTTATCTTTTTAAT
GCTGATGGGGGCCAAGAAAGGAGTTCATTTGCTTCCTGGCTGGGAAATTCA
GGAATTGCATTTTGGGCAGCATATTTTATAGTATGTGTGTAAGTATTTCAC
TGCTTTTCCTCAGTTTATTTTTTGTGAGAGTTCATTGAAAATGTCCAAGGC
TGGGCACAGTGGCTCATGCCTATAATCCCAGCTCTTTAGGAAGCCGAGGTG
GGAGGATCACTTGAGGCCAGGAGTTTGAGACCAGCCTGAACAACATAGCAA
GACCCTGTCTCTACAGAAATAAAGAAGGAGGCAAAGGCAG

In some embodiments, the intronic domain that promotes trans-splicing primarily in the liver comprises or consists of sequences from the gene MRPS27. In some embodiments, the sequences from gene MRPS27 comprise or consist of:

(SEQ ID NO: 608)
CTAGGCAAGCTCTGTCTTCACCTGAAAATCCTACTCAACACTGGGGACAGA
GATCTCTTTAGCCATGGGATACCAACTTTTAGAACTAGAGCTAGAACTTGA
GAGTACACTTTGTCACTCTTTCTTATGCTCCTCCAAGAAATACACATTTCC
TCAGAATTGTAAGTGTAATCACATTTTATAAGCTGAAATCTCAGAGTACCT
TTAGCCTTCATGAGCACAACCTTGTTGGGGATATTTGGGTTGTATTTGTTT
CTCTTTTCTACACTCACATGTTTGCCTATCTTGACAGCCTGTTGGCCTAGG
CAATAATAAAGCTTCTGTCGAGGTGAGCAAACAGCTGATGGCATAGGCGCA
CAGAGGGGACTCCTACAGTGGCTGGCTGTAATCTGCCAGGCTGGTGGTCAG
AGCAGGCGAGAACAAAAGCCAGTCTGTTTGCAGGCAAATTGGAAAATCAAT
GTGTTAGTTGTATTCTTTTCTCTTTGCCTTTCTTCTGACA

In some embodiments, the intronic domain that promotes trans-splicing primarily in the liver comprises or consists of sequences from the gene DMGDH. In some embodiments, the sequences from gene DMGDH comprise or consist of:

(SEQ ID NO: 609)
GTAAGCATATCATTTTTTTGAAATTAGTGTTTTTTAAGTGCCAAAGAAAGA
AGATATTTAGCCGTAATGTTTCGTACGATGGACATTGTCATGAAGCACTGT
GATGCTCCCAAGTTAAACATTTTGTCTGTTTCTAAGTGACAAAGCCCTCCC
TCTATCACTTTGCTCTTGGTGGATGCGCTATCACTCTGTCCTTGGTTATCC
AAAAAAAGTTGCTTATTTTCTCCATTCTGCATTTTATTTTGCTCCAGATTG
TGTTTTTCCAAATTATGTTTATTTTTCCTTCCTGTATTCACTGTTTCTCTA
TATGAATAACCAGATTCTAGTGTTGAAGTTCATAAGCATCCTCTTTGTGTC
CAATTTGCTTTTTAGGGCAGATTTGATCATTTGGGTTCTCTCTCATCCATT
TCTGATGACTTTTGACTCTAGTTGAAAAAGTTTTTAAATGCTTTTCCTTCT
AGAACATTATTAAACATATTTCATAATCTTCTCTTTTTGT

In some embodiments, the intronic domain that promotes trans-splicing primarily in the liver comprises or consists of sequences from the gene DMGDH. In some embodiments, the sequences from gene DMGDH comprise or consist of:

(SEQ ID NO: 610)
AATAGATGAGACCCATATATAATATTTCACTGATCATTCTATCAACGTATC
CCATCTGAGTTTCATTCTTCTTTGTTTTCCCCACAGTACCTTGCCTGAAGG
CCTAAAACAAAGTAGATATGCAAGAATTAATTGTTGAATGATTGAATCAAA
CATAAATTTCCTGACACCAGATATATTATTTAATGTGAATTTATTAACAGT
ATTAAAAATTAAATATAAATTATAATTGATATATCAGTTGGAATTAGATTT
GTCTTCAAATGTCAGAAACCAAAATAAGAAGGTGTAAACAAGATAGAGGTT
TATTTCTCTTTCTTTTCAGCAGTCCAGGACTTGAATGGTGGCTTTTTCATG
AAGTCTTCAGGGACCCAGTCACTTTCCAGATCAAGAAACACTTTTGAAGTC
CAGTTTTACTAAACGTTTACTAGCAGGATATATTTTTACTGAGTAATAAGT
ATATATATATTTGAGTATTGACTCTGGTATGATTGATTTA

In some embodiments, the intronic domain that promotes trans-splicing primarily in the liver comprises or consists of sequences from the gene ZCCHC9. In some embodiments, the sequences from gene ZCCHC9 comprise or consist of:

(SEQ ID NO: 611)
GAATGCTCAACGAGCACTGAAGGGGGGACATGAGGCACTGCCTCTCACCCA
AGAGAGCCCCCAAATGTCCTTTTCAGACTTTTATCACAGTGACCCACTCCC
ACCGCGCCTTTACCTTGCCTCTGCCTGGAATGTCCTTAACTCCCTCTTGCC
GGCAATTTCACCAGATTTTTGATTTCTTATCCAGTCTCACGGGAAGCCTTT
CCGGCATCTCCCACCCTACCGATCTTCCCGTCTATCTCGCCTGCTACACTG
TGAGTTCAGACAGCACGGATTGAACTCTTCATCTTTTCGTGTCGGCGTCCT
GCACAGGGCCATCCCATCACTAAGCGCGCCACAAACTCAGCCCAGTATTGA
CGCCCACGAGTCGCTTGTAAAGACCGTCTGTGACGCCGGAAGTGACGCGCT
CTCGGGCACTATGTGCTTCCGGTCTCCAATACCGCAGGAGGGCGGTCTTCC
CCGGCTCGCCAACTCGGCTGCTCTGGGGGATTCGTGCGCG

In some embodiments, the intronic domain that promotes trans-splicing primarily in the liver comprises or consists of sequences from the gene ZCCHC9. In some embodiments, the sequences from gene ZCCHC9 comprise or consist of:

(SEQ ID NO: 612)
TGAGGTTCTCAGCCACCAAAGCTGCAGGGTCTCGTCGTTCGTTCCCTCTGG
CCTGGCGGAACTTGCGTGGGAAGCTGGGAGGACAGAAGGCTTAGCCGCTGG
GTCCCTGAGCTGGCTGGAGCGCGGGCTGTCTAGGGGCGGCGACCTCAGCTG
TTGGGCTCCGGCTGCTGGGGGCTGTGCGTCCAGCCGCCCTGGAATCCGCCT
GTGGGTGGGCCGCTTGGCTCTCCCACTTCACCCTCTTGATAACCCAGAGCC
TCCCCCTTCCCACGTACGTAGGGGAAGGATAATCTACTTCCTTTTGAACAG
CTTCGGTCCCCACTATGACAGTCGCATAAACTTCAGCGAAGAGTGCGCTGG
TAAAGTGGCATGGAGTTTCTCACACCAATTCATTGGGTCCACCCTTCAGCT
TCTGTGGAAGGGGTCTTTCTGTTACTAGGAGCTGAGACTTGGGGGGTTGCC
AGGGATGCTGGTCTTAAAAGGTGATAATTTAGGCCGGGCG

In some embodiments, the intronic domain that promotes trans-splicing primarily in the liver comprises or consists of sequences from the gene LEAP2. In some embodiments, the sequences from gene LEAP2 comprise or consist of:

(SEQ ID NO: 613)
GACGATCTGGGGAACTGTAGGCCTTGCTGAAGCATCCTCCCACAGAAAGGA
GATAAGGGCCTTATCCAATTGGCTGTACACAATAGATCAGTGCTTCTTTCC
TAGACAAGGCTGAAAGGGGCCAACATTATTTCTGAAGACTTCATTATTGGA
ATTCTATGGGAGTGATCTCACTGAGCTATTTTGGAATAGAAATGTGGCTAG
TTGCCTGACCTCCCTCAATGGTTTCACGTGGCTTTCAAAGGGAAGGAAGGG
CAGTGCTGACTTTTGGTAAAATGGGCGAAAGGGTCCATGCCAGCAACACAA
TCACTCAAAGTCCAGATGAGGGATCAGTAAATACAACGTGCCTGAAAGGTG
GCCCTTGAGCACATTCCTCCGGTAGACATTAACTTATTAAATTGATTCTGA
TTACAAATATAAACTTTGCCCCCATCTCACCCAGTAACAATGCAAGAGTTG
ATGTCAGTCTATAAAAGGAAGTAGGAACTGTCCCTGGCTT

In some embodiments, the intronic domain that promotes trans-splicing primarily in the liver comprises or consists of sequences from the gene LEAP2. In some embodiments, the sequences from gene LEAP2 comprise or consist of:

(SEQ ID NO: 614)
TAAGGAGGGAAGGATACTTATGTGTGTGTGTGGAGTGTGGAGATGATAGTG
GTGGTGGAACTTGAAAGCTAGATTCAGTCCTGAGGAATGGTTCCTCTGTTC
TGAGTCTACAGCATCTGCGGAATGGAATGATCACTCTTCCAAGGTGTGCAG
CAGGGTGTCAACACTTTCATATCTGAATGTCTTTGCCCTTACAGATAGATG
GCTCCCCAATACCAGAAGTGAGTTCGGCAAAGAGAAGGCCACGGAGAATGA
CCCCATTTTGGAGAGGGGTTTCCCTCAGGCCTATTGGAGCCTCCTGCCGGG
ATGATTCTGAGTGTATCACAAGGCTATGCAGGTACTCCCTGAACCTGGGAG
CAGGGTTGGGCCAGAGAGCCCTGGGAAGCTGGGCAGAGGAGTGACAAGGGG
ACACATGAACCTAAGAATAAAGCTGGGATGGAGGAGTTCTAGACTGAGACT
GGGAGCTCCATGGAAAATCCCTTAGGAGTTAGGAGCCAAG

In some embodiments, the intronic domain that promotes trans-splicing primarily in the liver comprises or consists of sequences from the gene ETF1. In some embodiments, the sequences from gene ETF1 comprise or consist of:

(SEQ ID NO: 615)
GTAAGGGGCCCGCGCTCCCCGGTTTAGATCACTCGGCCCCCCTCGTGGTAG
GCCACCGCTACTTTTGCACCTCTCCCGGGCCCTTAAGTCCAGGTCCTCCCT
CTTCGGGCTCCAGGTGCTGCCACCCGACTCGGGTGCTATTGTGGCAAAGTG
TTCGGCCTTCCTTTCCCAGGGCCCTTCCCTGATTTCCTCTTCTCCCTTCAT
TTCTCAGCCAGGCCTCATTAAAATGGCCTCTCCCTTCTGGGCTTCTGAGAC
CCGGGTGGGTGAAGTTTGGATGGGACGGGAAGCAGAGGTGGCCAGGGACAG
CTTCTTTCTGATTCCTGATTTCTCTGGGTCTTCCACCTGTCCCAAGATGAC
TTCTCCCAAGTCCTCTAATTACCCCGGAGCTTCCTGGTTTTAGCTGTTCCA
GGACCCACCTATAAACTGAAAATGCCAACGTACGCGCGATGTGAGGGGTGC
TGGGTAGGTTTTGGTTAATGAATCTGCATGTGTATGTCCA

In some embodiments, the intronic domain that promotes trans-splicing primarily in the liver comprises or consists of sequences from the gene ETF1. In some embodiments, the sequences from gene ETF1 comprise or consist of:

(SEQ ID NO: 616)
GAGAAGTCGCGGCGGACGCCCAGCTTCTCCTCACTCGCACCGGCCCCCTCC
CTTCCGCCTGCCGCCTTCCACTCCGCCCCTGGCGGAGGAAGTGATGTCACA
GGCCCCATGTGAGCGGATTGCAACACATGCAGCTGCCTGGAGAGAGGGAGC
CGGTGTCCTACGTCAGAGCCGCCGCCGCCGCGGAGCCGCCGCCGGGGAGGA
GCAGCCGCTGCCGCCCAGGACTGGGCCCTTAGGTAAGGCACAGGGAGGGAA
AGGCCGGTGACCTTTGTGGCGAGCTGCACCGGACGGGAGGAGAGGGCCTCT
GGGGGATGGCGAGGAAGGGGGCCGCTCTGCCCCAGCGGCGGCGCTAACTCT
GTGGTCTTGGTGTCTCCGGGCCCCGCCGGGCCGCTCTAGGGAGGAGGAGGC
GAGAAGATGGCGGACGACCCCAGTGCTGCCGACAGGAACGTGGAGATCTGG
AAGATCAAGAAGCTCATTAAGAGCTTGGAGGCGGCCCGCG

In some embodiments, the intronic domain that promotes trans-splicing primarily in the liver comprises or consists of sequences from the gene RNF14. In some embodiments, the sequences from gene RNF14 comprise or consist of:

(SEQ ID NO: 617)
CATTGCTGTCTTTTATTACATCTTTTTATATTAAAAATAAATAAAAATGTC
ATAAATAGTAATTGTGTAAATATAAAAGGCCTTATTAAAAAAACAAATCTT
AGTGCCTCAGTTTTCTCTTCTGTCAAGTGGGCACATAAAATTACCTAAGTC
ATAGGGTCCTGGGCATTTAAATGAGAGAATGTACGTGAAATATTTAGCACA
GTGGAGGGCTAGGCACACGGGGAGACTGCAGTTTAAAAGACTAACCAGGCA
CATGAGACCAAAGACACAAAAGTCTGTTATTTTCCTAGAGTCAAAAACAAA
ACTTATTAGAAGGCTGTCTTTACCCAATCTTGTCCTCAAAATTGACTCTAA
ATCTTTAGTTCACTTTAAACATTCAGTAAATTATTAACACTGCTTTGAAAT
TCTCTTTCTATTCTCTCTATCCTGTGACCTCTGACATGATGTCACAATTCA
CCTTCTGCTCTGACTTTGTGAGGATTTCCCAAGCTGGTTG

In some embodiments, the intronic domain that promotes trans-splicing primarily in the liver comprises or consists of sequences from the gene RNF14. In some embodiments, the sequences from gene RNF14 comprise or consist of:

(SEQ ID NO: 618)
TAGGGTTGCTGTGTGTTTTGCTCTTGGTCTAATTCCTTTGGCTAAACATGA
ATAATTATATTAATGAATGGCTTTGGAGATTCTTCTTCCGTCCTTTGCATT
TTTCCGAGATATGTTTCTGGTTTTGGTATCTATTAGAAGGGTATTTTGGTT
TCTGTTTTGTGACAATCAAGATTTATAGTACAGAAAATTAGAAGTGATTAT
TTCTATTTCAAGGTTCTTTCTGTGGCAGCCAGGTAATCCCTTGGGTGCCCT
GATTTACTCACTTGGTTTTTTTCTCTCTCCTGGGTATAAGACCAACTACAT
AATTTACAAAGCCCAGGGCAAAATAAAAATGCAGCCTCTCATTCAGAAATT
ATTAAGAACTTCAAGAGGGTGAGAGCACAGCATCAAATCAAGAGCAGGGCA
CTTCTTTTTTATTTTAATTTTTCCATAAGTTATTGGGGTACAGGTGGTATT
TGATTACATGAGTAAGTTCTTTAATGGTGATTTGTGAGAT

In some embodiments, the intronic domain that promotes trans-splicing primarily in the liver comprises or consists of sequences from the gene NR3C1. In some embodiments, the sequences from gene NR3C1 comprise or consist of:

(SEQ ID NO: 619)
GTAGGTTATATTTTTATTCCCATTCTATAGCTGGGTAAAGTGAGTTCTAAC
AAAGTTACTTGTTAAAGGTCACTCAGAGGTCAGAGCATCAGAAAAAAAGAC
AATCACAAGGCTGATGTTGTGTGCTGGATAGTTTAAACTGAACAGGAAGAA
AACATTTTTGTGGGCTTTATCTAAAAAGAAATAGTTTGCTCTGAGTTTCTC
AGTTTCATTTATTCAGCAAGTATGTGCCAAGTGCTATTGTAGGCAATGAAG
ATACAGCAGGGAACAAAACAAAACTGACATGGAGCTTCCAACTAGAAAGGA
GAACGAGACAACGCGTTTTAAAATATATATAATGTGTTAGGTAAAAAGTGC
TATAAGGAGAGCTAAAACACAATGAGAGGCTAGAAGAGTGATAGTGGCGGA
GGGGCTTCTCTGAGGAGATGACATTTGATCAGGGGTCTGAATAAAGTAAAG
AGGCCAGTTATGTGAATATCTGGAAGACTCAAGGGGCAGG

In some embodiments, the intronic domain that promotes trans-splicing primarily in the liver comprises or consists of sequences from the gene NR3C1. In some embodiments, the sequences from gene NR3C1 comprise or consist of:

(SEQ ID NO: 620)
TAACTATAAATTGTGCATTCTTGCATAGATCCTCCTTGGGAATGAGAAATT
AGGAAAATCCAGTTGTTAAAATGAATGCCTAAAATCAAAATAAAATTTGTT
TTTCTGGCACCTGCTTGATGACACAGACTAATAACCAATGACAAAATTGCC
CTTGAACCCAAGTTTTCATTTCCTCCTATTGTGTGGTCAGGTTATGTAAGG
GTTTGCTTTCACCCCATTCAAAAGGTACCTCTTCCTCTTCTCTTGCTCCCT
CTCGCCCTCATTCTTGTGCCTATGCAGACATTTGAGTAGAGGCGAATCACT
TTCACTTCTGCTGGGGAAATTGCAACACGCTTCTTTAAATGGCAGAGAGAA
GGAGAAAACTTAGATCTTCTGATACCAAATCACTGGACCTTAGAAGGTCAG
AAATCTTTCAAGCCCTGCAGGACCGTAAAATGCGCATGTGTCCAACGGAAG
CACTGGGGCATGAGTGGGGAAGGAATAGAAACAGAAAGAG

In some embodiments, the intronic domain that promotes trans-splicing primarily in the liver comprises or consists of sequences from the gene ECI2. In some embodiments, the sequences from gene ECI2 comprise or consist of:

(SEQ ID NO: 621)
GTAGTGCTAAAAAGTGACGACCTCACGCGCGCCCTCCTGCACCGCTGGGCC
CGGAGTGGTCAGGATGGGCGCGTCGGGAGGTCAGGGCTATGGTTCCCTGTC
AGCAGTCCCCAAGGTCTCCGCCTGCCTCCGCGCACGCTCCCATCCTCCTGG
GGCCGGACTCCGGGCGGGTGAGCCGCCCCAGCTGAGCACCCGGGCCTGCAC
TTCCCAGCCGGGCTGCTTCTGGGGTGCAAAGCGTCGCTTGCTAAAAGCGAG
GTAAACGCAAGCCGTTATCACTCGTGTGGACTTAACACTCTGCTTCTTGGA
GAACACGTATGCTATAAAGTGAAATGGCCGGGTACAGAGTAACGGCTAAGA
AGCACGCTGCACCTCCCTGGGGTCCCGGGAACCCGCACGAATCTGTGCATA
GGGAACAAAACCGAAACACGGTGGTGGCCTTGAAGTGGTAGAATTATGAGT
GATTTTCCCGACTTTATGTATTTTCTTAAATGGTCGTGTT

In some embodiments, the intronic domain that promotes trans-splicing primarily in the liver comprises or consists of sequences from the gene ECI2. In some embodiments, the sequences from gene ECI2 comprise or consist of:

(SEQ ID NO: 622)
GGCCCCGCCCCCGCATCTGAGCCCCGACTCCTAACCGCCTAGAGCTCCCGC
CCCGCCCACGCACCGACCCCCCCGGCCCCACCCCCGAGCCCCCGCAGCCCC
AGAGGAGGCCACGGGCTAGCGATGGCGAATGCGGCGTGGCTACGCTCTCAG
CCTGGCCCCGCCCCAACCCTGGCCCCGCCCATGCCCCAGGCTCCTGCCTGG
GCTCTGCTCTCGGCCCCGCCCCGCACCCAAGCCCCGGCCCCACCCCCGAGC
CCCCGCAGCCCGGGAGCCTAGGCCCCGCCCCCGAGTCCCGGCTCCACCCCC
GAGACCCGGCCCCACCCCCGAGCCCCCGCAGACCGAGCTAGGCCCCGCCCC
CGAGACCCGGCCCCACCCCCGAGCCCCCGCAGCCCTAGAGCCGCCCAAGGG
ATGGCGATGGCGTACTTGGCTTGGAGACTGGCGCGGCGTTCGTGTCCGAGG
TAAGCTTGGAGTCCCGGCCGTTCGGCCCATGGTCGCCCGC

In some embodiments, the intronic domain that promotes trans-splicing primarily in the liver comprises or consists of sequences from the gene ALDH5A1. In some embodiments, the sequences from gene ALDH5A1 comprise or consist of:

(SEQ ID NO: 623)
TGTTCTTTGTGCTTCTTGTATTTGGATGTCTAGGTCTCTAGCAAGGCTGGA
GAAGTTTTCCTCGATTATTCCCCCAAATATGTTTTCCAAACTTTTAGAATT
CTCTTCTTCCTCAGGAACACGTATTATTCTTAGGTTTGGTTGTTTAACATA
ATCCCAGACTTCTTGGAAGCTTTGTTCATATTTTCTTATGCTTTTTTCTTT
GTCTTTGTTGATTGGGTTAATTTGAAAACCTTGTCTTTGAGCTTTGACGTT
CTTTCTTCTGCTTGTTCAATTCTATTGCTGAGGCTTTCCAGAACATTTTGC
ATTTCTATAAGTGCACCCATTGTTTCCTGGAGTTTTGATTGTTTTTTATTT
ATGCTATCTATTTCATTGAAAACTTCTCCCCTTATTTCTTGTATCTTTTTT
TTTTTTTTTGTGACTTCCTTAAATTGGGCTTCGCCTTTCTTTGTTGCCTCC
CTGATTAGCTTAATAAATAACTATCTGAATTCTTTTTCAG

In some embodiments, the intronic domain that promotes trans-splicing primarily in the liver comprises or consists of sequences from the gene ALDH5A1. In some embodiments, the sequences from gene ALDH5A1 comprise or consist of:

(SEQ ID NO: 624)
TGAGCTCATGTGATTTTTGGGGGGTGTTAAAGAATCTTGTTTTTTCATATT
ATCAGAGTTGGCTTTCTGGTTTCTTCTTATTGGGGTAGGCTCTGTCAGAGG
GAAGGTCTAGGGCTGAAGGCTGTTGTTCAGATTCTTTTGTCCCACGGGATG
TTCCCTTGATGTAGTAGCTCCCCCTTTTCCTAGGGATGTGGCTTCCTGAGA
GCCGAGCTGTAGTGATTGTTATCTGTCTTCTGGATCTAGCCACCCAGCAAG
TCTACCAGGCTCCAGGCTGCTACTGAGGGTTGTCCGCACAGAGTCCTGTGA
TGTGAACCATCTGTGGGTCTCTCAGCCGTAGATACCAGCACAGTATTTGGG
GTGTCTCCCAGGTCCTGCAGGAGCAATCTGCTTCCTTCACAGGATCTGTAT
GCTCTCCCAGTTTTCCTGATTTATTCCTGCAGTCGTTCTGGAGCAAAAGTT
CACAATGCAAGCCTCCACATGCTGTTTTGTCCATCCGAGT

In some embodiments, the intronic domain that promotes trans-splicing primarily in the liver comprises or consists of sequences from the gene APOM. In some embodiments, the sequences from gene APOM comprise or consist of:

(SEQ ID NO: 625)
CAATCCACCGTGCCCCAGCACCTGCATCATACCTGTTCTAAAGCATTTACC
AAGTTGTATTGCAATGGTTTGTCTACACAGCTAGTTTTCCCTCTAACGACT
TCTTCAAGATCAGGGGCTATGTCTTATTCGTTTTTTTATGTCCCCGGGGAT
TAGCTAGTTCTTGGGAAACAACTGGGACTTGGGATTCAAAACAGTTTGCTA
AGTGAATGAATGAGAGGCCCAGTCAAGCTACTTCCCTTCAGTGCTGCACAG
TGCAACAAATAACCAGCTCAGATACAGGTTCAAAGACCACAGGCTTCTCCC
TGGACAGCTCAGCTCTCCTCATAACTCCTGAGAAACCCTGGACATGCCAGG
GTGTACTATGGAGTGGTTGCATCCGGAAGAGGGGGAGGAAGTCCCAAACAC
TAAGTAATCCAGGTTTGGGTTGGAAAACAAGGTTGAAGTTACTCATTAGCA
GGTGAAAGGGTCAAGGGTCGAACGCAAGGGAGCTGAAAGC

In some embodiments, the intronic domain that promotes trans-splicing primarily in the liver comprises or consists of sequences from the gene APOM. In some embodiments, the sequences from gene APOM comprise or consist of:

(SEQ ID NO: 626)
TATGGACTGAGATTGGGGGAAGCCTATGGTGGAGGCTCTGAGGGACTTG
GGTGGATGGCCTAGGATGACTGGAGACCATCTTGGGAAAGGAAGAGAGG
AAGGGGGTGTGAGTGTTGTGATAATGAAAGCAAGAAGAAAAATATCAGT
ACTGTGGCCATCAATGCAGAGGCATGGCAGAATTGGGGGGTGGGGTGGT
TACCCAGGTTGACTGGGGAGGGGCAAAGAGGAAAAGTCATTTAATGACT
CTTTGTCATGGATCCAATCCCCAGTTGGAAAGAGGAAGGCAGCCAACAC
CTCTACCCCTAAATCTTGCTGTTTTGACTGATGAAGAGGTTGAACCCAT
CCTGTGCTGGAACCCACCCTCTTTTGCTCCCTTCATTGTCTCTCCAGTT
CCCAGAGGTCCACTTGGGCCAGTGGTACTTTATCGCAGGGGCAGCTCCC
ACCAAGGAGGAGTTGGCAACTTTTGACCCTGTGGACAACATTGTCTTCA
ATATGGCTG

In some embodiments, the intronic domain that promotes trans-splicing primarily in the liver comprises or consists of sequences from the gene FKBP5. In some embodiments, the sequences from gene FKBP5 comprise or consist of:

(SEQ ID NO: 627)
GTAGGGACCCCATGGAGTACGTGACGCCTGGCCCTCCAGCTGCATGCTC
CAGTTATCAGGACAGAGACCCCTCCCCACCCATTCCCAGGCTTCCCTGG
CTCCCTGCTCTGCTGCTGGGAGGCCCTGTCACCACACATGCGCGTGCAC
GCACTAACATACACACACCCCTCTCAGGCACCCCCGGAAGACCAGCTGC
AGCTGGGGGTGGGGGTGCACGGAGCACAGCCAGGGTTCTCAGATCCCTA
GCCTGCTCTCCCACCCCGCCCTAGCCCCCAACTGCCGAGCTGCTTCTAG
TTTTAAAGCAAAACATGACTCTGTCCTTCCTGCTGCTCAGTGGCAGCAA
CACTGCCTGGGGTTATTTTTAGCAGGGATGCAGGGAAGGCACAGTTCTC
CTTGACTTTAACTAGGGACAACCCCCACCCCTCAGCCAAGACTCACTGG
AGGAGGCCGGGTACGGAAACCCCCCTCCCAGAAAGGGGAAATGGTGTGT
TTTCTGTTC

In some embodiments, the intronic domain that promotes trans-splicing primarily in the liver comprises or consists of sequences from the gene FKBP5. In some embodiments, the sequences from gene FKBP5 comprise or consist of:

(SEQ ID NO: 628)
GGACAGTCATCTTGGACATGCTCTGTGTTCCATATCATGACTTCAAGCC
TTGCCTTTTTCATGTACTACCAGTGGGATCCTAGACTAGCACTTTACCA
CTTTAAGCCTCAGTTTCCTAATTTGTAAATAAGGAGTAATTGTAATCAT
TCCAATATCACAGGCTTGCTGGGAGGATTAAATGAGATCATCCACATCA
GGGTCAGCACAGTGCCCGGAATGGGGTACCTGTGAAATTAATGGTGATT
CTTCTTTATTGTTACCTTGGAAAATGGAGGCCAACTGCAGGATGATCGT
GGCTCCAAGGAGCTGGGGACCCAAGACATGCACAGAGAAAGAGAGACCA
ACAATGTGAGGCCTCTGATGCATGAGACAGGCAGGGGAGTAGTGGGAGG
AGAGAGCAAGAGTGAGCAGCCAGGAGGGCCTCTTCCCTTCCCTTTACTC
AGCCTGTCACTCCTCTGCTAGGATCCTGCATTGACACATCTTGTACTTC
CTGTTTCCA

In some embodiments, the intronic domain that promotes trans-splicing primarily in the liver comprises or consists of sequences from the gene SLC22A7. In some embodiments, the sequences from gene SLC22A7 comprise or consist of:

(SEQ ID NO: 629)
AGGTGTGTCTTAGGCCAGGGCATCAGCTCCCAGCAGGGGAGAGGGATGC
GAGGAGGCCTCATGGCTCAGCAGAAAGAAACCTGGTTAGGATCCAGCTT
TCAGTCCTGAATCCTGGGCTGTCGGCCAGGCCATGGCTGTAGCTGCAAA
TAGGCAAGGCTGACCTAAGGTCTCACAGGACAAAGGGCACCCTGGGAGA
GCTGGCCCCAGGCCCCAGGGGCCTCCTCTTCTTAGGGATCCTGGGAGCT
CTGCTGACCAAGGGGCTAGGGGCTCACCTCCTATGCCATCACCCAGCTA
CCTCATCATGGTAGCCTTGGTCCCTGAACACTAATGGGTAAGCCCCAGA
GCCACGAGCCAGTGACCTCTGAATGGATGGCTTCCCCTTCCCCCAGGCA
CCAGGCAAGACAGGTAGAGGTCCCTGGAGACCTGATGGCTGGGGAGGGC
CCAGTCCTGGGCCAGCCCCTGAGGGCTGGATTCTGGCTGCAGGCTCTCC
AGTCCATCC

In some embodiments, the intronic domain that promotes trans-splicing primarily in the liver comprises or consists of sequences from the gene SLC22A7. In some embodiments, the sequences from gene SLC22A7 comprise or consist of:

(SEQ ID NO: 630)
TACTTAAGCCCAGAAGTTGAGAGACATGTGAGAGGGATGGGCCTGCCAT
TGCCTTGGGTGGTTACTGTGTAGGCATTAGATGTATTACTTTACCTCTT
AAAGTCTCAGTTTACCAATCTGTAGATGGGGGCTATGAATATAAGGCAC
TTTTAGTTCATGGCTTGTAGTAACAGGATCCTACACAAGAACTGTTATC
ATCATCATTAATTGGGAGGTGATTAAGGCAGGGAAGGGCTAAAGTGGCT
TCAGGGAGTTGAGACAAAGAGCTGAGAAGGCAATTTCTGCAGCAAGAGG
TGGAGAAACAATAGAGGCCTTCTTTTCTCCCTTCCTCTTTTCCAGTCCC
AGGTCGGTATTTACATAATCCATCTGGAGGTGGAATGTCGGTGGAGGCA
GTCTCCCTGGGAGGCAGCAGGTCGAGGCCTTCCTTGGGAAGAAGCTGAG
GCTGCAGGACTGGGGAGGGACAAAGTTTCCTATTCCCCAAGCTGGCGTG
AATCGTTGG

In some embodiments, the intronic domain that promotes trans-splicing primarily in the liver comprises or consists of sequences from the gene MLI P. In some embodiments, the sequences from gene MLIP comprise or consist of:

(SEQ ID NO: 631)
TCACTACAGATGCTCCTGTATTGTTGCTTACAATTTATATACTAAGGAT
AAAGTTACTGATATTTTGAAATAACTGTGTGTTTTCTAGTTCTTTATTT
GGACACAGTGTTTGCATGTATTTATACCTCTAAATGTTCTCTATGAGCA
TGACCAACTGATTATTGAGATTGGCAGGTGCTAGCGCCCTTCAATCAGC
TTCTCATTGCTGCACAATCTGGCTTTTCTCTTCTTTTCACAGAATATAT
AATTGGAATACATGCATGAAAAGAAAAGAGGGAAAACACATATCAGTGG
CTTTTTTTCTTTTTCTTTTTCTTTTTTTCACTTCTACTGATTCCATTTC
AGCAGTTAGTATGACCTCTCATAATGTTCCAAATCTAAAAATTGTCAAA
ACAGAAATCTACTCTTCGGAGCTGACACAATTCCTCAATTTTGGAATGA
CTCTGCTTTACCTCTTTTTAGAATTTCTTCTTTCTGCGTGAGTGTGTGT
GTGTTGTTT

In some embodiments, the intronic domain that promotes trans-splicing primarily in the liver comprises or consists of sequences from the gene MLI P. In some embodiments, the sequences from gene MLIP comprise or consist of:

(SEQ ID NO: 632)
TAAAAGGAAGTCTTTGCTTAATGCTTTCAGTAACTTTTAAAAGCAGTGT
ACGGTGCTGGTGGTATTTGCTGCAAGGATGTGAGGGAGAGCTGCCCAGT
GAAGATAAATTTGTATGTATATTGAAATGAAATGCTATCTTCACTTAGA
GAAAAGGGTGAGCTGATAAAGACAAAACTGCTCATTGGGCAAAGAAAAT
GCTCAGGATTGCCCTTGGCACAGTGATAATGTAAGATACCCTGTAATGC
CTTAACAGGAGGCTAGAGGCCATCTGCGAGTGTGGGTGTCAACATCTGA
AGGAAGAGAACTTTTAGGGAAATATAAGAAAGGAAGGCATTACAGCCCA
AAACTGAGCAAAGGAGCTGTTAGCCTGCGTGGCATGCGGGAAAACAGTT
TCTCAAATTATTAGGCTATCCGGTGAGAAGATGACGTTCTTAGAGATTT
TGAGCATTTCAAAGTGTATTTAGAAAAAGATTTTAATCCTTTTATCTCA
TATTTCTAT

In some embodiments, the intronic domain that promotes trans-splicing primarily in the liver comprises or consists of sequences from the gene ENPP1. In some embodiments, the sequences from gene ENPP1 comprise or consist of:

(SEQ ID NO: 633)
TAGGAGCAGCGTGCCTATGGAAAATAGAAAATAGGTTCTTTTTGTTTTT
GTTCTTGTTTTTTGCTTCAGAAATAATTATAGAAATCTGTGTTTAGAAG
CAGTCCTAAGAAGTTACATGCAAAGAAGTACCAGTCTTGGAATGAGAGT
GAATCATACACATACACTCCCAGTTAATATCCTAGGTGGTTTGGGGCAA
ATAACTGGGCTTTCACATTCGGTCTAAAAATGTGATAAGACATTCTTCT
TTGCTCACATAATGATATTGTAATATATAAATAAGAAGAAACATTAACC
AAAATTTGGGTGACGGAAAAGATTTTGAAAAAAGTGAAGTGATAGGTAC
AGCTGAAATTCTGTCTTACCTATCAGATCTTCAACTAATATGAGTGCTA
CACCCATGTTTAACGAATTTAACCTTGGAAGTGAAAGAAGTTCTGCTCT
GCATATTAAATTTTTTGTTAAAGTTACAGCATGTTTTGGGATTTTTTTT
TTCTCCTAG

In some embodiments, the intronic domain that promotes trans-splicing primarily in the liver comprises or consists of sequences from the gene ENPP1. In some embodiments, the sequences from gene ENPP1 comprise or consist of:

(SEQ ID NO: 634)
TAAGTATATCTCTGTGATAACTTTGAATATGGTCATATTAAGAATACCT
TCCTTTAGGCCGGGCACAGTGGCTCATGCCTGTAATCGCAGCACTTTGG
GAGGCCAAAGTGGGTGGATCACCTGAGGTCAGGAGTTCGAGACCAGCCT
GGCCAACATGGTGAAACCCTAAAAATACATATACGATACTATACTATAT
CTATACTAAAAATACAAAAATTAGCCTGGCTTGGTGGCAGCTGCCTGTA
ATCCCAGCTGCTCTGGAGGCTGAGGTACAAGAATCGCTTGAACCCAGGA
GGCGGAGGTTACAGTGAGCTAAGATCATGCCACTGTACTCCAGCCTGGG
CAACTGAGTGAGACTTGGTCTAAAAAAAAAAAAAAAAAAAAAAGAATAC
CTTCCTTTAAATCATTATATCGCTAAACTAAAGGGAATCACAGTTAATT
TTTTTATGATTAAATCTCCAATGCAGAGCTTTTGTACACAAATGGATTT
TATGGGACT

In some embodiments, the intronic domain that promotes trans-splicing primarily in the liver comprises or consists of sequences from the gene VNN1. In some embodiments, the sequences from gene VNN1 comprise or consist of:

(SEQ ID NO: 635)
GTAAAGAAACAACTTGTGAAAAATTCACTAGTAAACATCAACTTGATTT
ACCTGGGAAAACTTTGTTGATGATCATTGCATAGATCCATGATCAATTC
TTAAGTTTCAGTATAGCTTATTTTTCATCTACTATGGGTATATTTACTG
GGAGAGCAAATATGAATTATGAAGTCACAGAAGTCAGAGCTAGAAAGTA
GCTTAGAAATCATCACATTCAGTGTGAACATCTCTGGTCTCTGACTCCT
CACCAGTGAACAGAAAAATATTTCCCTGTGTAGGTCTGTGATTTGAAAA
CTATATGAGTAAATGGCAAAAGAGAGTCACATCAGTTTAAGATTAATAG
TTTTCCTTTCTCATTGCTAAGATAGCTGATGAGGTTAATGTAGTAAAGT
CCTTAAAGTGTAAGCTGATTGTAATCTAAGAGGTGATATGGCAGGATTT
TAAGTGGTTTAAGTCAGGTCTCGGCTACAGAGATATTAAGTGTGGTGAA
AGCAGCACT

In some embodiments, the intronic domain that promotes trans-splicing primarily in the liver comprises or consists of sequences from the gene VNN1. In some embodiments, the sequences from gene VNN1 comprise or consist of:

(SEQ ID NO: 636)
CATCTCTACTAAAAATACAAAAATTAGCCAGGCGTGGTGGCAGATGCCT
GTGATCCTAGCTACTTATGAGGCTGAGGCGGGAGAATCGCTTGAACCCG
GGAGGTGGAGATTGTGGTAAGCCGAGATCTAGCCATTGCACTCCAGCCT
GGGCAGCAGAGCGAAACTCCATCTGAAAAAAAAAAAAAAGAGAGAAAAA
AAAATGCTTGACTAGAAGCCCAAACCTCACCATTATGTAACATATCCAT
GCAACAAACCTGCATTTGTACCCTTTGAATCTAAAATTAGAAATAAAGA
AAAGAAAAGAAAAAGAAAAAGAAGTGACAGTGCACTGAAAAAAAAGGAA
ATTAAAATGCTTTGGAAAAGAAAATAAATTATAAAAATATAGAAAACAA
AATAAGATTTAAGGGGTGTGGGGGAAGCCCAAATAGTTGTTACTCAGCC
ACTCAGCTCCTCAGCTCCTCTTGCAGGCCCCCCTTTGGATTAAGTTGCA
TTTTTAACA

In some embodiments, the intronic domain that promotes trans-splicing primarily in the liver comprises or consists of sequences from the gene SGK1. In some embodiments, the sequences from gene SGK1 comprise or consist of:

(SEQ ID NO: 637)
GTTCGTACTTACCGTACTATATTTTGCAGATGCCTCAAAGGATTTGGGG
TTACTTGGCATGGGGAAGGCACATAAGGTGGGGTGTAGGAGAGGGTCTC
TGGTTGTAGGTTTCTTAATTTAATGTTTGAAAACAAACATGCAAAAGTC
TGTGTGCAGGTTGATGTTTCTGGGCAGCCTGAGCAAAATTTGCTCTCTC
AAGAGGGAAAGGAACCAGGTGGGAGCAGAGCTAGGCTGGGCTAGGCTAG
TTGAATGGTGGGACATGACATACGGGTGGCACTGGCAATAACAAAGTCA
CATTCTATGAAGATTCCCTGCAAGAGGAAGCAGACATGGGCCAGTTACT
GTGATTTGAAATTGCCTAAACATTGCTTTAGGTTGGCATGTCAATTTCA
GGTACTAGTGTTTTTTTTGTTTTTGTTTTTGTTTTGTTTTTGTTTGTTT
GTTTGTTTTGAGACGGAGTCTCGCTCTGTTGCCAGGCTGGAGTGCAGTG
GCGTGATCT

In some embodiments, the intronic domain that promotes trans-splicing primarily in the liver comprises or consists of sequences from the gene SGK1. In some embodiments, the sequences from gene SGK1 comprise or consist of:

(SEQ ID NO: 638)
AGTAGGTTGCTGGGACAGAACAGGGGGCGCCTGCTCCACCCATCACGTA
TCCGGTTGGAAGAAAAGCCAGTCTCAACAACTTGATTCTCTCGGTTAAA
TGATAAGTTTAGCCGACCATCCGTCTCCAAGTGCTGAACATTAGGGTAG
GGGAGGAAGGAGCACATCAGACGTTCCCTTTCTTTTCCAACCACAAATC
ATACGGTTGCATCTATTCTCTATTCCGCGTAGAACCCTCCGACCTGAAT
GCCACGTTCCTCCCGGAATGCATGCCACAGAGCCTCTGAGAAAATAAAT
CCTGTGCATGTGTTTTGAATTTTTTCCGGACTGATTTGTGAGGATAATC
AGTTAAGTTACATTTCTTGTGTGCGTGCTGGGGGTGTAATAAAGCTAGG
ACACTGATGACTGTAATTTTCTGAGTGATGTCGCAATGGGGAGAATAAA
TGGCCCAGTGTTGAACTTTGCAGCTTGCTCCCTTCCTGTACTTAAATTG
CCTTGATAT

In some embodiments, the intronic domain that promotes trans-splicing primarily in the liver comprises or consists of sequences from the gene PLG. In some embodiments, the sequences from gene PLG comprise or consist of:

(SEQ ID NO: 639)
ATATCCCCAGAGTGGGTGTTGACTGCTGCCCACTGCTTGGAGAAGTATG
TTTAGGGGACAATTGACATGAAGTCTTGTCTTAAATACTTTTTCTGTCC
TTCTTTTCCTCCTTTCCTCCTTTCCTTTCTCACTCTTCCTCCCTTCCTT
CTCTGGCTGTGACACTAGGGACCAGGCCAGGGCAATTGGATAAGAGAGA
AGGGAAGGGTTTCTAGAAAGAAACTGCAGAGGAAAGACACAGTACAGAT
GATTTTGTGGGCCTGAATAAACTGCAGAACAGAGCTGTTCACTACCATA
GGCTGTATCAGTCTCTGCCCAAACAGCCCAAGAACATTCCTTAACTGCC
TGTTTCAAGCAAATCATGAATTTTGCTTCTTGCCACTCAGAAGTCACTA
ATTCTGAGTGGCCAAGGGTGTCAGGGAGACAGCACCAATTTCATGGCAC
AGAGGTTACCTGAAGGGGCTGGACCATATTTTCCTCTTGACGTCCTCAT
CTTTTCTAG

In some embodiments, the intronic domain that promotes trans-splicing primarily in the liver comprises or consists of sequences from the gene PLG. In some embodiments, the sequences from gene PLG comprise or consist of:

(SEQ ID NO: 640)
TACTCGTTCACCTGTGGTCTTCACCCCACGCTGGTGAAGATATTTGCTT
TATGTCTGGGTTTTATGGGCCATGGCCACTGCATGGCAGTGGGGAGGAA
CTGTCTATCACATGAAAGGCTCAAGGGCTTTGGGGACAGCATCAATCTT
CAACCCTAGCCCTGCCACATGCTAGCTGTGCTCTTGAGAAAGGCAGCAG
GACTCCGTTTTCTCATGTGGAAAAAGAGTTGAAATGAGGTACTCTGTTA
CTCCTAGAACTCACTTAATGTTCACCAGTTCATACACATTCATGATCAG
AGAACGATTCAGTTATTCCAGGCTGACAATTCCCCCTTCATCATAATAT
GTTTAAGAGAATCATATAAGACTATATTTGTTTCAAAGCACTTTAAAAA
CCACAAGATCGAGTTGGGTGTCTGGTGTGGGTGCCTGTAATCCCAGCTA
CTTGGGAGGCTGAGGCAGGAGGGTCACTTGAGTCCCGGAGTTTGAGGCT
GCAGTGAGT

In some embodiments, the intronic domain that promotes trans-splicing primarily in the liver comprises or consists of sequences from the gene ABCB4. In some embodiments, the sequences from gene ABCB4 comprise or consist of:

(SEQ ID NO: 641)
GGTCTCGAATCCTGACCTTGTGATCCGCCTGCCTCGGCCTCCCAA
AGTGCTGGGATTACAGGCGTGAGCCACCTTGCCCGGCTGATAGTG
GTTATTTTGAAGTAATGGAATATTTTATTCTAAATTTTCTAAAAT
GATTATATATTTTATAAAGTAAAGCATTCAAAAGAAAAAAGCTGC
ACAAATTACATTTTTAGCACATTAGGAATGAAAAACTGGAAAATA
CTCAAAAGGGAACTAGTTAAACAGAAGATTAGAAAATTTAAATAC
TTATGAATTTTTTTACAGCATGGGAACCCATTTGTGTTATATTTA
AAAAGCAGAACATAACTATGTAGATTAAATGTTGATTATACAAAA
TACACATGCATAAAGTTCAAAGATTCTAAGATACTAGAAATTAAA
AGTTGATTAGAAAGGTAACATTTTCAAATTTACTTGAATGTTAAA
ACTAATTTATACAATTTTTGGGATAAGGTGTCTGTCTGATTTATC
AATA

In some embodiments, the intronic domain that promotes trans-splicing primarily in the liver comprises or consists of sequences from the gene GSAP. In some embodiments, the sequences from gene GSAP comprise or consist of:

(SEQ ID NO: 642)
GTACGCAAGTATTTCTCACTTTTATCATGGTGTACAAAGAGTGGA
AACAACTCTGTTGTAGCAAAACCCTTTTGAAATGCACTAGTTCAA
ATCAGTTCTCACCAGCTTCAACACCATCTAGCTTAATTCTATACT
CAGCATTTAAAGTTATGCCCCCCTGCATATGTACCCTTTAATGTA
TAATAAAAGCTAAAATTAAAACAAAGTTATGCCCGTTACTTTACC
CATCCTCTTTCCCTGGCACATGGTTGGTGACAGGGGAGGGACAAA
TGAGCCCAGTACTGCCAAGAAGGGGAATTTCACTTAATACAATGT
GTAACATATGTTCCTACTCTGATTAAATTCTAGGCATTAGGCCGA
TGGTCACATTCATATTTATGGGGCTTTCTACAAGGCATAGCATTT
CATGATTAGCATGAACCCTTGTGTAGTACTCAAAGCCACTGCTTA
GGACAAATGGAGCATCACAGATGAGCTCCTTGGCTGACATGCTGC
TTTG

In some embodiments, the intronic domain that promotes trans-splicing primarily in the liver comprises or consists of sequences from the gene GSAP. In some embodiments, the sequences from gene GSAP comprise or consist of:

(SEQ ID NO: 643)
TGAGGACACTGCAATTTGGAGAGGGTGGGTAACTTGTGTAGGGTA
ACACAACTAGTAATGTATAGAATCTGGACTTGATACCAGTCTTAC
TTCACAGCTCATGATCATGACCTCCATTCTGTGTCTCTTTTCTTC
TGACTGTCCACTGTTGGCTCTTGCGTTGTGACAGGCCTGGAGCAA
ACCCTGTAGCACTTTACCTTCACTGATGGATGGGGGTCTGTAAGT
GTGAGTGGTGGGGCCAGTTAGCTGGGCTGGGGAGGCTGAGCTGGG
GCACTGCCCACACAAACAGAGGATGCTTTGTTAGTTTCTGGTGCA
CATTTTCAGTCTTTCTATTTTGGGTTCAGTGCTTCAGAATTGACC
TCAGAAAGGCTAGTTGTGTGTCTGGATGCAGCCTGAATGCCAGCT
GCCCTGAAATGCTTATCTGAGTTGTGGAGGCCTAGCTGTGGGGCC
CTTGAGGGAAGGAATGGAGCAATGGTTAATGGACTGTCTTCACCC
TCCA

In some embodiments, the intronic domain that promotes trans-splicing primarily in the liver comprises or consists of sequences from the gene ABCB4. In some embodiments, the sequences from gene ABCB4 comprise or consist of:

(SEQ ID NO: 644)
ATCTGACTTGTAAAATAAGTTTCTGGTTTGTCATCATAAAACATA
GGTATGTCTGTCTCATTCTAGATCAATTTAGAAGACCCAATTTGG
GGTTTATTACGGCATTTGAAATTTGAGAGTTAGAAGTGACACCTG
TCTTGATGAAGTATTGAAACAACAGGCTAGCTCTCCCTAAGTTAT
TAGAAACATCCAGAAAAAGAACAAAATTCAAGGTAAGTACAGGTG
TAGAAATATAGAAGCCACAGACATAGGGAAATGGTCTTATGGTCC
TGAATGGGTTGGGTAGACAGGAAAGTTTGGGAATAATTTTCCCAA
GTACAAAAGAAAAATAAATTGTTCAATAGCCTCATCTCTCTTGGT
AATTTTCTAAATCATTCTATTGTGGAATCAAAGTTTTTTAAATGA
ATGAATTTCCAGCTTTATCATCCAACATCAAGAGAAAATGAAATT
GCCCTACCGAGCTAACTCTGAAAGCACATATCGGTTCCATAAAGC
AAGA

In some embodiments, the intronic domain that promotes trans-splicing primarily in the liver comprises or consists of sequences from the gene STEAP1. In some embodiments, the sequences from gene STEAP1 comprise or consist of:

(SEQ ID NO: 645)
AAACTCTGTGTTGAAAAAGTCATCTATTTTAAAATTTTTATTATA
ATTACACTAGGGCTTGGTTGTCAGTCGGTTTTATATTGAGAAATG
TTTTTTAGGCTGAAAATAAGGTACAACTTTAGCAACATAAGAAAC
ATTTTGTTCATGGACGAGGCCATATTTCAAACTTCTACCACCCTC
ACAAGATTACTTTTTAATATTGTTGTTCTTCTCCTTGTTTACCAA
TATGCATGCACATTACAGTTCTATCCAAGTAAACCATCAAATAAT
TATTAGAAAATTAATAGATTATGGAAGTGTTGCATTCAAGAAAAA
AAGTGATAGCTCCTATGTCATTTGTCTTAGAGGAATGCAAAAAAA
AAAAAAGAAAAAGTCATAAATTTGTGGAGACCTGTTATCAGGGCT
TCATAGTAGGCACAGGGAAGAGTGTAGAAGGAGATTCACATTCAG
GAAATAACTGTTGTTTGCATTTCTTCTTTCTTTATTTACCTTCTG
GTAG

In some embodiments, the intronic domain that promotes trans-splicing primarily in the liver comprises or consists of sequences from the gene STEAP1. In some embodiments, the sequences from gene STEAP1 comprise or consist of:

(SEQ ID NO: 646)
TAAATAATATATAAAATAACCCTAAGAGGTAAATCTTCTTTTTGT
GTTTATGATATAGAATATGTTGACTTTACCCCATAAAAAATAACA
AATGTTTTTCAACAGCAAAGATCTTATACTTGTTCCAATTAATAA
TGTGCTCTCCTGTTGTTTTCCCTATTGCTTCTAATTAGGACAAGT
GTTTCCTAGACATAAATAAAAGGCATTAAAATATTCTTTGTTTTT
TTTTTTTGTTTGTTTGTTTTTTGTTTGTTTGTTTGTTTTTTTGAG
ATGAAGTCTCGCTCTGTTGCCCATGCTGGAGTACAGTGGCACGAT
CTCGGCTCACTGCAACCTGCGCCTCCTGGGTTCAGGCGATTCTCT
TGCCTCAGCCTCCTGAGTAGCTGGGATTACAGGCACCCATCACCA
TGTCCAGCTAATTTTTGTATTTTTAGTAGAGACAGGGTTTTCCCA
TGTTGGCCAGGCTGGTCTCGATCTCCTGACCTCAAATGATCCGCC
CACC

In some embodiments, the intronic domain that promotes trans-splicing primarily in the liver comprises or consists of sequences from the gene PON1. In some embodiments, the sequences from gene PON1 comprise or consist of:

(SEQ ID NO: 647)
GTAACAATATTTACTATTTATTAAAACCAAAACCAAACTTAAAAC
AAAAACAGAAAAACATGCCAATAAATGAATTATTTTTTCTCATTC
AAACAAAGCATAGTCATGAATAATGTTCTTACTCTGGGTTTTAAA
CTTACCCTCTATGTTAATTGAACTTTATAGTAAAACCACTTAAAT
ACTTGAGAATGCTAAAATATTTAAGATTTGTAAAGCATCTAATTT
TGTAAAATACTAACATTCACATTATCCCTATAATTACCATAATAA
TAATATCCAGCCCTGATCATTATAGCTTTTCCATTTTAAACGATT
TTCAAGACTCGGGCATTTACAGGAACAGATTTTTCTAACCCAAGG
CATCTGTAAGCTATAGGTCCAGTGACAGAAAGTCACCATGGTCAG
CTTTCTGCCGGTCCCATTATCTTCAATGCCTCCTGTCCGCTGGTT
CCTCTGTTCTGTGTTCAAAATGCTCCTCTGCATGTTGGTCCCTTT
CAAC

In some embodiments, the intronic domain that promotes trans-splicing primarily in the liver comprises or consists of sequences from the gene PON1. In some embodiments, the sequences from gene PON1 comprise or consist of:

(SEQ ID NO: 648)
TCTTATATGGGAACACAATATATAAAACAACAGCTTTATTGTTTT
GATTGAAGGCTAAGAAAGGAGTAGTTCCTCTGGCCCACCAAAACT
CTGTCTTTGAGATGCTTCCAAGGACCTACCAGGGAACAGTTTAAA
AGCCATAAGCAATATTCTCTCTGTATTCCTTGCTTATATCATTGA
AGACTATTATTAATTGCCTTTTTGGTCTGCTCTTCCATTTTTGAT
CTTTCTCATGGGCTGGAAGCTGTAGTGTCGTGCACATATGACTTT
GCTGCAGGGGATGAAAGGGTTTGTGTGGGGTGGCCTAGTGGAGCA
TGTGGCTTCCAGAAGACTTCTCCTGGGGACCCATTACAGAAGCTA
ACTAAGAGGCTTCCTATGTGGCCATCTGGCTAGCCCTGTGGTTTG
GAGCAGTCAACATGAACCCATGGGCCATGCAGCGCCTCTATTAGC
ATTTGAAGTACTGGTATTGTGCTTGATTTTTCTCCTCCATGGTTT
ATAA

In some embodiments, the intronic domain that promotes trans-splicing primarily in the liver comprises or consists of sequences from the gene TFR2. In some embodiments, the sequences from gene TFR2 comprise or consist of:

(SEQ ID NO: 649)
GTGAGTGCGCGCGCGGGCGGGGGGCGCGCGGGGAGGGGGACGCGG
CTCGGGGCGCTCACCAGCCCCGCTCCCCAGGGCTCACCCCAACAC
CCTGCACTGGGTCGATGAGGCCGGGAAGGTCGGAGAGCAGCTGCC
GCTGGAGGACCCTGACGTCTACTGCCCCTACAGCGCCATCGGCAA
CGTCACGGTGAGCACCCCCTGCCCGCTCCTAGGATGGGGCGCGGC
ACGAGCCCTTTCCTGGGTCTCGAAGGAGGGACGCGGGGAGCGCGC
CTCACCGTCCTCGTGTCCCCAGGGAGAGCTGGTGTACGCCCACTA
CGGGCGGCCCGAAGACCTGCAGGACCTGCGGGCCAGGGGCGTGGA
TCCAGTGGGCCGCCTGCTGCTGGTGCGCGTGGGGGTGATCAGCTT
CGCCCAGAAGGTAAGTGTCCCTGGACAGAGGGAGGGCTTGAGCCC
GGGAACCGGAGGGTCGGACTGCCAGTGAGAATCGTTCAGGGTGCT
GTGA

In some embodiments, the intronic domain that promotes trans-splicing primarily in the liver comprises or consists of sequences from the gene TFR2. In some embodiments, the sequences from gene TFR2 comprise or consist of:

(SEQ ID NO: 650)
AGTCTCCGGCGCCGCCCTGGTGGTGATGGGAGAAAATGGGTCCGC
GGAGGGCCCTGCACTGCGGCTCGCGCCTGTAATGCCAGCACTTTG
GGAGGCCGAGGCGGGCAGATCACCTGAGATCAGGAGTTTGAGACC
AACCTGGTCAACATGGTGAAACCCGTCTCTACTAAAAATACAAAA
ATTAGCCGGGCGTGATGGCACGTGCCTGTAATCCCTGATACGAGG
GAGGCCGAGGCAGGAAAATGGCTTGAACCAGGAAGGCGGAAGTTG
CAGAGAGCCGAGATTGGTCCACTGCACTCCAGCCTGGGCGACAGA
GCGACACTCCGTCTCAAAAAAAAAAAAAACCGCCCCTGCCCCTGG
CGACCCCACGTCTCTGGCATCCTTCCCTCTTCCCTCCCTCTCCTC
CGGGCGCCCAGAAAAGTCCCCACCTCTCCCCGCTTA

In some embodiments, the intronic domain that promotes trans-splicing primarily in the liver comprises or consists of sequences from the gene SLC39A14. In some embodiments, the sequences from gene SLC39A14 comprise or consist of:

(SEQ ID NO: 651)
ATCTCGGCTCACTGCAACCTCCGCCTCCTGGGTTCAAGCGATTCT
CCTGCCTCAGCCTCCTGAGTAGCTGGGATTTCAGGCACGCGCCAC
CACGCCCAGCTAATTTTTTGTATTTAGTAGAGATGGGGTTTCACC
ATGTTAGTCAGGCCGGTCTCGAACTCCTGACCTCAGGTGATCCAC
CTTCCTCAGCCTCCCAAAGTGCTGGGATTATAGGCTTGCGCCACC
GCACCTGGCCTACTTGTATGGTTTTTAGGCTTCCATTGCTGTTTG
CATCCGTCTCTCCATGGAAAAGGTGTTTCTCTTGGATAGCAATTT
TGATAGTTTCCCTAGGCTTCTCCGCAGCATCTGGTGAGAGATGGA
CTCAGCAAATGTTTACTGATGGGTTGGTGGATCTCAGTCTGCCCC
ATCACACCTGAATTCAGAACAGGGCCGCCCCAGGCAGCAGGAGTG
TCCCCACCCTCAGTAATAGAGGCCCTCGTCTGTTCTTGTTCCTCC
ACAG

In some embodiments, the intronic domain that promotes trans-splicing primarily in the liver comprises or consists of sequences from the gene SLC39A14. In some embodiments, the sequences from gene SLC39A14 comprise or consist of:

(SEQ ID NO: 652)
TGCAGTAGAACAGAAACTTGCGCGTCCTCTTTCCCTGGGGCGGCA
CAGCTGAGCCAATCCCCTCCTCCCCTACCCTGGGCTCCAAGGCAC
CCAATGACTGCCACCTGAGAAATGAACCTGTCCCTCGTATCAAAG
CCACAAACGACTTCTAACTCTGGATCTCAGGGCCCACCTCTCACT
CCTCTGGCCTCTGCAGGTGCTGTCAGATGCAAGGTTCTTCCAATT
TCCCTCCTCCTGGGGACTTGACAGTCTCCCCTCCCTTGTTAAGCC
CCATACATAAGGCATCAAGGGAAAGCAGCAGCTATCGACTGCCGT
CCATGCCTTTGGTGCTAAGTACTTGCTCTATTCTAAGGAGAAATC
CTGGCAGTACTCAGGTCTGGGCACCAAGAGCTTATCCATCTGGAA
TGAGATGGAAGGAAAAGAGGTTACCAAGTGATATGTAGAATTGGC
CTAAAAAGCGAGTGTTCTTTAATTTTGAGCAGGCCATCGTTTCTA
GCAT

In some embodiments, the intronic domain that promotes trans-splicing primarily in the liver comprises or consists of sequences from the gene RBPMS. In some embodiments, the sequences from gene RBPMS comprise or consist of:

(SEQ ID NO: 653)
CCTTAGGAGTCGCCCAGCAGCGAGACAGCTGTGGATCCCCTCCAG
GCGCATGGGAGGCTCACAAGGCAAGGACTCTGATGGTTTTGGCTT
CCACCTCCTGTACGGTGGCCAGTGCTGTGAGGGTGGTTGGTCCCA
CAGGGAAACCTGTGAACAGAGTCATGTCTGGGGCCCCAGGGACAT
GGCTTCAACCCCTCACCTGGCAGTCCTGCCTCCCAAAAGTAAGGC
AGGAGTCCGGAGATTCCCATTGCTTGCTTCTGTGAATCTCAGGTC
ACTTTAGCACTTGACCCCTGACCCCAGACTGCCTTGTCCTTGAGG
CCTGGGATTCCTAAGGTATATACTGTCTGCTCCAGAGCTGTGGCT
GTGGCTATCCGGAAAACGACAGCTTTGAAGGACTGCAGAGCTTGC
CTTTGGGGCTGTTCTGCCCAAGGCCTTCCATTGTTTTCAGACATA
AATGAAATTGTTAATCCTCTGACATTTACCTTTTCCTCTCAACCT
GCAG

In some embodiments, the intronic domain that promotes trans-splicing primarily in the liver comprises or consists of sequences from the gene RBPMS. In some embodiments, the sequences from gene RBPMS comprise or consist of:

(SEQ ID NO: 654)
TGAGGCTTTCTAGGAGCACCTGGCTTAGCTCTCACAGGAGGAGGG
GCGGACGGGGAGGCCAGGCCTCATGCTCACAGCGCCAGCCTCCTG
TGAGAGTGGGCTGGGTCTCCTTTCCTTTCCCAGGCCCTTCCTGGA
GGCGATTTCTTTCTTTCTTTCTTTCTTTCCTTTTCTTTTCTTTTC
TTTTCTTCTCTCTCTCTCTCTCTCTCTCTCCCCTCTCTCCTCTCT
CTCTCTCTCTCTCTCTTTTCTTTCTTTTCTTTCTTTTCTTTCTTC
TTTTTTTTTTCTGAGGCAGAGTCTCGCTCTGTCGCCCAGGCTGGA
GTGCAGTGGTGTGATCTCGGCTCACTGCAAGCTCCGCCTCCCGGG
TTCATGCCATTCTCCTGCCTCAGCCTCCCAAGTAGCTGGGACTAC
AGGCGCCTGTCACCACGCCTGGCTCATTTTTTGTATTTTTAGTAG
AGACGGGGTTTCACTGTGTTAGCCAGGATGGTCTCGATCTCATGA
CCTT

In some embodiments, the intronic domain that promotes trans-splicing primarily in the liver comprises or consists of sequences from the gene DPYS. In some embodiments, the sequences from gene DPYS comprise or consist of:

(SEQ ID NO: 655)
GTAAGTCTAAGTTCTTGTATTCCTTGGCTTCATTGTGGAGAAAAT
GCACTAACTTGCACAGAAATAATGCTTTGGCATTAATCAGCCGCT
TCTCCTTAGATGACTTCTTTAGCTGTGCTCAGGTATTTTGATCCT
GTACACAAGATAAAGGTTTTGATGTGCAAATATTCCTTTGTTTTT
CTTGGCATAGAGGATGTAGAAGTTGTGTTGCATTCTCTGACAATG
GATCTGTATATAAAACCAATATGAATTTACCACTTCAGTGCCTGG
CAAATGCCCGAGTTCACATGGAAGAAAGGTAAAGCGTGATGAAGG
AAATAAAAGGCTTAGTTTGAAAAGGGTTGAATTGAAAACTCAAAA
TAATTACAGCCTTTTTAAAGCTTTCAGATAGAAAAATTCCAGCAC
AAAGGATGTCACTTTCAGTTAGCCAAATTTTCCAGTTGAGTTCAT
CTTCAGCCTATCATTCCTAGTAGATTCCAGCTTTTTGTTTCCAAT
TTCC

In some embodiments, the intronic domain that promotes trans-splicing primarily in the liver comprises or consists of sequences from the gene DPYS. In some embodiments, the sequences from gene DPYS comprise or consist of:

(SEQ ID NO: 656)
GCACCACTGCACTCCAGCCTGGGCAACGAGAGTGAAATTCCATCT
CAAAAAAAAAAAAAAAAAGTGTTGTCACCTATTCTCACTCTATAA
TATTCTATCTAATGTCTACATGATTATCTCATAAATCTTAGGTCT
GTAATAAATAAGTCAGATCATAAATGTGGTATTGTCAAACTTTAA
AGGAGAATCGAAAATACCATTACCAAAATTTAGCTGTCACCTATA
ACAATTTTTGTGATTCTAGGATGAATGGGAAGAATTAGTGTGGAT
GCTAAGTAGTGGGGTTTCTGAAAAGTCATCTTCAGGCTTCCATAT
GTTACCCCATCATCTCCATCATTGGCATTATGACTGTTTAATACT
TACCAGCATCCTCAGATGCTCTACAATATAAGAAATTAGGTGCAG
TTCTGTTTGTTTAAGAGATGCAAGTCTTGTCATTTTATCGATAGG
ATACTTTTTAGTATTTGATCATTAGAATGATTCTTTGAATTGCTT
TACA

In some embodiments, the intronic domain that promotes trans-splicing primarily in the liver comprises or consists of sequences from the gene RCL1. In some embodiments, the sequences from gene RCL1 comprise or consist of:

(SEQ ID NO: 657)
TATTGGGATGGGTGAGAGGAGGAAGATGCTTTTTGGGAGTCAGAT
CTGTTTAATTTTGAGCAAGTGTCTTAGTTTTTTTGTCTTAGTTGT
CTTTAATGTAAGTCCTAGAACTCGGAGCCATGGCCCTCAACACAG
GGTTACTGTGAGCATAAAGTAAGGTGATAATAGATGTGAAAGAAC
TTTGGGAGCCGTGAGCTCTGTGGGATTATGTGCTAGGATGGTGAC
ATCTCTTGTGCTTTCCTGGGGACAGTGGGCAGCCTCAGACCTCAG
AAGGACAGCACCTGCTTGGTCCAAAGGGTATGAGATTCAGGACTG
TTTGTCTAAGCTGTCTGTGGTCAAAAGTACATTCTGGACTGAAGA
GATGGTGATGTAACTGGGCTGTGTGTGTATTGAAAAATCCCTCTT
TGCTCTTTCCTCATTTCAGGTTTGTCCTTTGAGTCAAGTTAAATA
TTTATTGAGCATTTCTGAGTACAAAAGTGGTGTATTGGAGAGTTG
GGAG

In some embodiments, the intronic domain that promotes trans-splicing primarily in the liver comprises or consists of sequences from the gene RCL1. In some embodiments, the sequences from gene RCL1 comprise or consist of:

(SEQ ID NO: 658)
TAAGTTCAAGTGGA
GCTAACACTTGGGTGCCGGCTGGAGGGTTTCAGGAGAGAGATT
GGGACCTCGGGCTGGGAGAATGGGGCAAAGCAGCTCAGGTTGAGG
AATTTGGTCAGTTTGGATGGGTGGAAGGAGTTGATGGTGCAGAGC
TGAAGATGGACACAACTGAGCAGGCTGGGGGCTCTAACAACAAGA
TATGGGAGGACCAGGAGCCACCGAGAGACTGTCACCAGCATGAGG
AAGATTGGTCTTTTAGTATGAAGTTCAGGATGGGTCAAAAAAGGG
AGCTAGATACCAGATCAGGTCATGTATCGTCTCTGAGCACTTTGT
TTCTGCAGTTCAGATGAAAATTAAATATGTTATTGTCAGCAGTTC
TTGAACATTGGGCTGGATTGGGCAGATTATATACCTCGTTCTACC
TGTTGGACTTGTCACGTGGGGTTTGTATCAGGGTAGTGGGAAGGT
CACATGTTATTTCTCCATTTTTTTAGTTCCAAATATA

In some embodiments, the intronic domain that promotes trans-splicing primarily in the liver comprises or consists of sequences from the gene TOMM5. In some embodiments, the sequences from gene TOMM5 comprise or consist of:

(SEQ ID NO: 659)
GTAGCGGGGAGCAAGGAGGGTGCGCGTTGAAGGCGACGGGCATAT
GCGCGGTTTGAGGTCTGCGGGTCAGGGTCCGGGCACTGAAGTGCA
CGTGGTGGCCGGCGCGCGTGTTTAATCATCCTGGTTCTCCCAGCT
TGAAGAATGTTTGAGCTGCCGGGCGCGTTGGCTCACACCTGTAAT
CCCACCCAGCACTTTGTGAAGCCGAGGCGGGCGGATCATTTGGGG
TCAGGAGTTCGTGGTGGCGGGCGCCTGTAATCCCAGCTCCTCGGG
AGGCTAAGGTGGGAGGATTGCTTGAATCCAGGAGGCGGAGGTTGC
AGTGAGCCGAGATCGCGACACTGCACTCCAGCTTGAGTGACAGAG
CAAGACTCCATCTCAAAAAAAAGAATGTTTGAGCTGAAAGAGCTC
AAGTAATTATCCCTAGAGGAACTCCCTCGTTTTTGCAGACAGAGA
AACAGAGAAACAGAACCATAGTTCTAGAAGGAAGAAACAGCCTTC
TTGG

In some embodiments, the intronic domain that promotes trans-splicing primarily in the liver comprises or consists of sequences from the gene TOMM5. In some embodiments, the sequences from gene TOMM5 comprise or consist of:

(SEQ ID NO: 660)
AGGCTGGAGCGCAGTGGCGCGATCTCGGCTAACTGCAACATCCCC
CTCCTGGGTTCAAGCGATTTTCCTGCCTCAGCCTCCCGAGTAGCT
GGGATTGTAAGCACGCGCCACCGAGCCGGGCTGATTTTTGTATGA
CAGATGTTATTAAAGACAGCGTGGAGAAACCAGAGCCTGCGTTGC
TGAGCTTGCGGCCGCCCGCTTCACACCGCACTTCAGATCAGCTGG
GTCAGGACGTGGATTGTCCCGTGCAGGCATCCATCGTCGAGTGTG
GCAACGAGAGGGCGGGAACAGGAAGTGAGGATAGGCCGAGTTCCG
GGCGCGAGGCGGCCACCGTGGAGAGCAGAGCGCGGCGGCTGGAAG
CTGCTAAGTCAGAGCCGCGATGTTCCGGATTGAGGGCCTCGCGCC
GAAGCTGGACCCGGAGGAGATGAAACGGAAGATGCGCGAGGATGT
GATCTCCTCCATACGGAACTTTCTCATCTACGTGGCCCTCCTGCG
AGTC

In some embodiments, the intronic domain that promotes trans-splicing primarily in the liver comprises or consists of sequences from the gene CBWD5. In some embodiments, the sequences from gene CBWD5 comprise or consist of:

(SEQ ID NO: 661)
GTAACTAACCATCCCAGTCACAAAATGCCGTGAACTCTGGTGTCA
TGGGATTTTGCGGGCTGCCGGTGCCTCTTCTCCTGAGGCATTGGG
GCCTCATGATCAAGAGCAAATGTCTTTGAGGTAGGGGCTGGGTCA
CTTGCTTCTCGTATGGGTTACCTCTCATCTTGGGAGTTAATGATG
CCCGGTCCCCTCTGCGTTAGATTCCCATTTCTCAAGTGAGATGGA
AAGTTAGCCTTTGAAACAAGGCTTTGAAGCTAAGACACGCCCCCC
TAATTGTCTCTGTGACCTCTTACCAAGTTATCTTACCTTCCTGAC
GCTCATCTACCTCACCTGAAAATGGAAAAATACCTGTCACCTGTG
AAATAGGGCCGGAACAACCTACCTATTGCCATTAAAGGATGTCCC
GTCTGTAAAATGTCCGACACGTGGGGCTTTCGTCAGCAGATTCCC
CTTCCCCAGGAGCCAAAGTCCTCCAGCCAGTTGGCAGGAAGTGCT
CCTA

In some embodiments, the intronic domain that promotes trans-splicing primarily in the liver comprises or consists of sequences from the gene CBWD5. In some embodiments, the sequences from gene CBWD5 comprise or consist of:

(SEQ ID NO: 662)
CTGAGGTCCAAATAAATAATGAGGAAAAAGTAAATAAAAGTGATG
CATGAATACTAAAAATTTGGAATTACATATATAATATGTAATAAT
AAAAAAAGAAAAAATTGTCACTGGTTATCCTTCAGGTATAAAAAT
TACAATTAAAATTGTAATGTTATAACCTTCCATTCTTTCCCCTAT
GAATTAAAACTTTTTTTTTTAACGAGACTAAAAAATCAAGTTGAT
AAAATCTATGCCCTGTGGGAACTGGTTAGGTCAGGTTACCCACTC
CAGATTTCCAGCAGGCTAGCCTCAGCTCTACCTACAGCCAGTAGC
TTATTAAGGCTCTAGGCGCCAGTGACACCTTCAGACAGGTGGTTA
GTGCGCCCTAAGGGCTCTCCGCAGCGTCGCTCAGGTTCACAGAAC
ACGCCCAGGGGCGTGTCCAGCTGTCGTCGGGGAGAGCCCACCTCC
CCGGGGGGTGTGGCTAAGGGACGAGGCAGTTCTCGTCCAGAGCCC
AGGT

In some embodiments, the intronic domain that promotes trans-splicing primarily in the liver comprises or consists of sequences from the gene TJP2. In some embodiments, the sequences from gene TJP2 comprise or consist of:

(SEQ ID NO: 663)
AGAAAAGCAAGACAGACTGAGAAAGTGTGGCTTAGCCGCGGTATG
GAGGATGGATGGTAGCACAGATAGGCTGCAGGGCCCCTAATTTGA
GTTCCTGCACAAAAACGACCATAATATCAACATTGTACCAAAGCC
AAGTGATCTCAGGGAGAAAAATAAAAGATGCCTTCCCTGGCGAGC
ACATCAAGGTTTCAGTCGCTTGGCTGTGTCCTTGGGAATTTTCTT
GAGTCCCCACTCCCAGGAGCCACATCAGACCCCACTGACCGTCCA
ACACAAATTCCTCTAGATGGAAGGGATGGATGATGACCCCGAAGA
CCGCATGTCCTACTTAACCGCCATGGGCGCGGACTATCTGAGTTG
CGACAGCCGCCTCATCAGTGACTTTGAAGACACGGACGGTGAAGG
AGGCGCCTACACTGACAATGAGCTGGATGAGCCAGCCGAGGAGCC
GCTGGTGTCGTCCATCACCCGCTCCTCGGAGCCGGTGCAGCACGA
GGAG

In some embodiments, the intronic domain that promotes trans-splicing primarily in the liver comprises or consists of sequences from the gene TJP2. In some embodiments, the sequences from gene TJP2 comprise or consist of:

(SEQ ID NO: 664)
GCATAAGGAAACCCAGCCCAGAGCCACGAGCTCAGATGAGGAGGG
CTGCTAGCAGCGATCAACTTAGGGACAATAGCCCGCCCCCAGCAT
TCAAGCCAGAGCCGCCCAAGGTACGTGGCTGGGAAGCCCAGGATG
GGAAGGAAGAGGAAGCAGATGCCTCTGAAGCCTCCTGGACGGCCA
GGGAGGAGCATGCACACTGAGATGGTGTTTAATTACGGTTCTGAC
TCACTGTGTTCTCTATTAGAGCCTATCTTTTGGCCTGTTGGGTGA
ACTCCCAAATTAGGGAAGGGCAAAAAGGAAGGAAAGGCAATATTA
GGAGAGCATATGATTTTAGGACTGTTAGTGTTTATTTAATTTACT
CTTCTTGTTAAAAAAAGATTGTAAAGAAAAAGATTGAAAAATACT
GTACTGTATACCCAGAATCACCAATCGCAGAACGTGTTTTTCTTT
TGCTGTCCAGCTAAAGTTTTCTGCCCAGTCTGATTTAGATGCACC
AGAT

In some embodiments, the intronic domain that promotes trans-splicing primarily in the liver comprises or consists of sequences from the gene ALDOB. In some embodiments, the sequences from gene ALDOB comprise or consist of:

(SEQ ID NO: 665)
GTAAGGATATTGCCCACTGAGATGAATCATGCCAGTGTAAGCTAG
AAATGAAGGCAAGTAAAGAAGGAAGCAAGGAAAAACAAAACACAA
CTATTACACTGGTCTTAGAGCCACACGTACCTGGTTTTCAATACC
AGCTCCTCCATTTAACAACTGTGACCTTGGGCCAGTCACTGAAAT
ATTGTGAGCCTCAGTTCTCCTATCTCAAAATGGGGATTTCTCAAA
ATGTTGTGGGGATTAACTTAGACAATGTACATAAGGTGCTTACCA
CAGTACCTTGGACAAAGAAGATTAAACTCTGCACCCGGAAGTATT
ATTTTTAGAAAAAAATTAGCAGCAGTAGAGTGGAAAGTCCCCACA
CTTTGAGGTCAGACACACCTGGGTTTGAATCCTGGTTCTATCACA
TGCTACCTAGATAATTTTGCAAGGTACTTCTGCCCAAGTAGACAA
GCATTAACTCACAACATTGGGTTAGCTAAGCAGAATTTGAATCCC
AGCT

In some embodiments, the intronic domain that promotes trans-splicing primarily in the liver comprises or consists of sequences from the gene ALDOB. In some embodiments, the sequences from gene ALDOB comprise or consist of:

(SEQ ID NO: 666)
ACTTCTCAAAAGTGAAAACTAGTATCCATTGAATAAGAGTTTTTA
CCATTCCAAGCATTCATTTAATTCTCATAACCTCTGGTAGTAGAC
TCAATTATTACCCTTATTTAGAGATGAAGTGAGAAAACTGAACTA
CGAAGAGTAACATAACTTGCCCACACAACAAGGCAAGGTCAGAGC
CAGTTTAGGCTAATTCTCAAGCTCATGACTTTTGCCTTTGCTATG
TAGAGAGTAGAGGTTGTATCCTTAATTCTCTTTACCTCCTGTACT
AACACAACACCTGGTGCATGCAGGTGCTCAATAAATGTTAGGGTA
GACACTCTCACAGTGTACCTTCTAGGTAACTTCCCTTCTGCTATG
GACTGAACTGGGTCCCTCGCACTAATACACTTTACTAATTCAACT
CCTTGATCTGTAAAAGATGCAAACTGTTAGTTAGTTCAAGAGTTC
TGCTTGTGTTTTCCTGACATCTGACACCTGTTAATTATTTGTATC
CACA

In some embodiments, the intronic domain that promotes trans-splicing primarily in the liver comprises or consists of sequences from the gene PTGR1. In some embodiments, the sequences from gene PTGR1 comprise or consist of:

(SEQ ID NO: 667)
GTAAGTAGACCTCCTGCTTTCTTTCCTTCCCTTTTCTCCCTCCTC
TAACCCCCTTGCTTTCTTCCTGCCAAGTTTCTAGACGCCCCAGTG
GAGGAGGTTTGCTCTTAGACCTCCTCCCCTTACGTCGAAATGACC
GCTGGGTCTTGCACTCGGAGGATCTTGGGAGCCGACGTCCCGACG
CCTCCCGCCCCCGCAGTTCCTTGGAGAGCTTGGAGCCGCGCGCCG
GAGGGAATAGGAAAGCTTGGTTACAACCCGGGACACCCGGTAGCT
TCCTGCACCCGGGAGGGGGCTGGCCAGGGAACTTGCTGGACTGCA
CAACTGGGGGAGGGTAGAAGAAGAAAGATCGGGGTGGGGATTCGG
GAATGGGCAAGCGGGCGGCGGGCGGCGGGAGGCGAGGGTGGGAGC
AAAGGGCGAGCGGGCAAAGGCCGGGCGCCGCGCCAGGCCCCCTCC
TCGGGCGGGCGAGACTTTCCCGGGACCTGGCCCCTCTGCGGCCAG
ACCC

In some embodiments, the intronic domain that promotes trans-splicing primarily in the liver comprises or consists of sequences from the gene PTGR1. In some embodiments, the sequences from gene PTGR1 comprise or consist of:

(SEQ ID NO: 668)
GAAGCCAGGTCAAGGCCTGATGTTTGCTAGGGTGTGATGGGTGGG
GTGCCTCAGTGGCAAGAGTGCTCACTCCTGCCTGGGGGAGTCTGG
CAAGGGTTCACAGAGGTGACCTTGAATTGGCTGGTGGAAGGCGAG
TAGGTGTTCAGCAGGCGGACTGGGGACAGACAGGCAGGGTGTCTG
CACCGCAGCCCTGGGCGAAAGCTTGCACTGCGGGCAAGCGGCAGG
GCTGACGGGGAGCCCAGGGATCGGAGCCAGCACCGGGGGCTTATT
AGGGCTGCGGCCGCCCCAGGCCCTGCCTTTGGACCCTTCCGGCCT
GCTTGGCAGATGCCACGGTTGGTTCCCCCACGGGGCATATTTCCG
CCGGGCTCCAACTTAGGACCCCAGGCGGAGGTCCAGTGTCAGCTG
GACAAAGCTCTGTTAGGAATTTTCCTATCAGGAGTCCCTAACCCC
CTACACGCACACACACGGGCGCGCGGGCGCGCACGCACACACACG
CACA

In some embodiments, the intronic domain that promotes trans-splicing primarily in the liver comprises or consists of sequences from the gene ORM1. In some embodiments, the sequences from gene ORM1 comprise or consist of:

(SEQ ID NO: 669)
AGCCTCCTACCTCTGCTGGAAGCCCAGATCCCATTGTGTGCCAAC
CTAGTACCGGTGCCCATCACCAACGCCACCCTGGACCGGGTGAGT
GCCTGGGCTAGCCCTGTCCTGAGCACATGGGCAGCTGCCTCCCTT
CTCTGGGCTTCCCTTTCCCTGCTGGCTGTGGTCGGACCCCCACTC
CCGGCTCTGCCTTTTTCTCTTCTGGGTCCCCAGGGTGAAATTCTC
ACCAGCCCAGGGGACTCTGGAGGCACCCCCTGCCTCCAAACACAG
AAGCCTCACTGCAGAGTCCTTCACGGAGGACGGTTCTGTGCTGGG
CCTGGAGGGGCTGCCTGGGGGGCAATGACTGATCCTCAGGGTGAG
CTCCTGCATGCGCACTGCCCACCAGGGGCCTCATCTCCCCATCTG
CAAAATCAGGGAGAGATCTGCCTGAGTCTCCTCCCAGCTGACAGT
CAAAGATTCAGCATCAAGCCCCCATCACCAGCTCCCACCTTCTCC
CCAG

In some embodiments, the intronic domain that promotes trans-splicing primarily in the liver comprises or consists of sequences from the gene ORM1. In some embodiments, the sequences from gene ORM1 comprise or consist of:

(SEQ ID NO: 670)
TGAGAGCCCCCATTCCAATGCACCCCCATCTCAGCTTCTGGCCAG
AAGACCTGAGCAAGTCCCTCCTTCTTCCTGGCCTTGGCCTTCCCA
TGGGTGGAACCGGGAGGGCTGGCTTTAATCTCCACCAGACTCTTG
CCCCAGGACTGTGATGGGCGATTGGCCACTTCTCAATAATCTTCC
TGTTTTCCTTCCGCCTTCTGTTTGGCTTTAGACAGGACCAGTGCA
TCTATAACACCACCTACCTGAATGTCCAGCGGGAAAATGGGACCA
TCTCCAGATACGGTGAGGGCCAGCCCTCAGGCAGGAGGGTTCACT
GTGGGAACAGGGCAGGCCAGCATAAGGTGGGGGCTGGATGTAGAG
CCCTGGAGGCTTTGGGCACAGAGAAATAACCACTAACATTTTTGA
GCTCTAACGATGTGCTCAGAAACAACTCTAAGAGGACACTGAGAG
AATTAGAGGAGGAAAAAGAAGAACAGAGACCTCAAATAGTTTCCC
CAAG

In some embodiments, the intronic domain that promotes trans-splicing primarily in the liver comprises or consists of sequences from the gene ORM2. In some embodiments, the sequences from gene ORM2 comprise or consist of:

(SEQ ID NO: 671)
TAGTTTCCCCAAGGTCACACAGCTTATAATTAGAACTAGAATTGG
AACTCCAGGCTGGCTTCAGATCTGCCTCTCTCTCACGCCCTCTTT
AAGATCCTTTGCAAACCAATGGTAGAAGCCTGTATGTTGGAGAGG
TGGTACCTTCAACTATGTCCCCCATCACCGCAGAGGTGGCACATG
GCAGGGATCTGATGGAGCTGAACTGACATCATTTAGCATCCCGAG
CCTCCTCTCTGGGCCTCATTTTCCTCCTCTGTAAAACGGGGAGAA
AGGCCCTGACAGCCACAGTCTGTGTGAGGCTCCTGAGATCTCATG
TACAGAAAGTGCTTGGCGTGGAGCTGGGCACGCAGCAGGGGCTGG
GCACACGGTGGCCCAAAGGAGACCCGGGCCTTCACTGATGGGCTT
TGTGGCCCCGGACACACCTAGGACTCCTCACCTGTAAGACAGGCA
CCATTGTGCCATCCCATGTTCTCACCCAGAGGCTCTTTTTCTCTT
CCAG

In some embodiments, the intronic domain that promotes trans-splicing primarily in the liver comprises or consists of sequences from the gene ORM2. In some embodiments, the sequences from gene ORM2 comprise or consist of:

(SEQ ID NO: 672)
TAGGCATGCTTAGCAGCCCCAAACTCATGCCCCTCTCAGGCCTCA
CCCCCCATTCACCCACCCCTGGGCTGGCCCCTAGAACCCCAGCCC
TCCCTGGCCTCCCGCCGGGCCCCACCATGTCCCCAGTCAGTCTCC
TTGCTCCCCCTGCAGCTGACAAGCCAGAGACGACCAAGGAGCAAC
TGGGAGAGTTCTACGAAGCTCTCGACTGCTTGTGCATTCCCAGGT
CAGATGTCATGTACACCGACTGGAAAAAGGTAAACGCAAGGGATT
GGACAGTGCCCACCTTGTCCATGGCCCAACTTGGGCAGCCCCAGA
GGCCCAGAGCAGGAAAGCTGCCAGGCAAGGCTGCACAGCTAGGCA
GATCTTCTGCTTTTAGGCACCTGCCTCACTGTAGGGACAGCTGAG
CTCTACAGAGGCCCAGGGGTGGTGGATGAGAGCCCAGGAGGGAGA
AGTCCCTGTGAAACCAGGGAGGACCTGAAAGCTAACAGGAGGGAA
CAGC

In some embodiments, the intronic domain that promotes trans-splicing primarily in the liver comprises or consists of sequences from the gene C5. In some embodiments, the sequences from gene C5 comprise or consist of:

(SEQ ID NO: 673)
GTAAGTAAAACAAGCTATCTTCATTTTTCAATGCATCTTAGTTAT
GACGAATGAAGAGTTAAATTTAACAGATTCTGTTAACTTGAAAAT
AACTTGCTACTTCTGAGAACAGCAAGTGAATAGTATCATCTCTGA
ATGAGAAAGTCATGGAAGCATTTTGATTTGATGAGTAGCTAACAA
CATGACAGCTTTTATGGAATTGTTATTTATTAAAATTTACGTTAA
GTGATTATTCAAATTCCTCTTCAAAAAAGCACAAAAGAAACTCCA
GATAATAAACTTTCTTGACATTTTTTGCTTATTTTAACTTGGTGT
TTAGTTTTCTTTGTGTTTTTCATAGACTATACACAGTTAAACCAG
GGTTATTTAGGAGTCAATCTAATACTGTGATCTGAAAATCTGTTG
TTGTAGTGACTTCTACCATACATTTATGAGCTGACTTTTCACAAT
TTTTAGTGTCATTATTGAGATAATATGCATTTTAAGTAATGTTTT
AAAA

In some embodiments, the intronic domain that promotes trans-splicing primarily in the liver comprises or consists of sequences from the gene C5. In some embodiments, the sequences from gene C5 comprise or consist of:

(SEQ ID NO: 674)
CTAGGTTATGCTCAGACCATTTGCAGCACCAAGGACACTGCCATT
CAGGTCACCAAAAAAAGATCATCTTGTGAGCCCTGTCTAAACCAG
GCCCCTTCTAAAATCATTTCAGTGTGAAACTGAGTCCAGGTGGGT
CTTCATCAGTATTAGAGTGGACCTGCCAGATGTATGCTTGCAGAT
GATGCAACATCAAAGCAAATGCCCTGGGCAAGAGAAGAAAGGCCA
AAGCAATGGGCTTACTGGTTTCTAGCTTTGAGCCACTCTTCATAG
TGCCAAATTCCCATTTTAGGGTCCCTTTATTTGTATTAATAAAAA
TAAATTCCCTGGGTGTAAGTTCCTGTAAAGTACTCCCTATCTGTA
TTCCTGGGCATGAAAGGGTGTTTTAGTTCCAATTCTAGTTTTCCC
ACTTAGAGGTTCACCTTTCTGACATTAATAATTAACCAGATTATC
TGAATTCTTCAAGTTCAGTTTATTTAAAAGGAGACTATCCTCAAA
AGTG

In some embodiments, the intronic domain that promotes trans-splicing primarily in the liver comprises or consists of sequences from the gene DMD. In some embodiments, the sequences from gene DMD comprise or consist of:

(SEQ ID NO: 675)
CGTCTTCACCTCTTTTCAACCTGCTCCTCTTTTTTGCCTTAACTC
TGATGGCAGTCACACAACTGTGTGTTTTTCTTTTAATTCACTTTA
CACCTTGGTTTGGCTATTGCTTTCCATGGTTCATACTTAATTGGC
TGGATTTTATTTATTTTGACCTTTCAGTAAATTTTTTTGCGGCTG
AGTTTGCGTGTGTCTCCTTCACCACCTCATTTTTTGTTTTGCAGT
CCCGTTACTCTGATCAACTTCTGGCCAGTAGATTCTGCGTGAGTA
CTTTTTTTGCTGAAGGGTGCTGCTACCACCAACACATTCGCTCGC
TTGGTTCTCTTTTGTTCTGTTTATTTGTTTGTTTTCTTTTGTTTT
GTTTTGTTTTATGGACATATTAATTCCCCTTCCTTCATTTCAGTT
TTTTTTTTTCTTTTCTTGATGAGACTTTGAAAGTACCATCTACAC
AGATCAGGAGAATCAGAGTGGGTTTTTTGGTTCCTCCACCAGGTG
AAAT

In some embodiments, the intronic domain that promotes trans-splicing primarily in the liver comprises or consists of sequences from the gene DMD. In some embodiments, the sequences from gene DMD comprise or consist of:

(SEQ ID NO: 676)
AAAATACCATAAAGTGTGGTAAAATTTAATGTCACCTTTTTGACC
TTAAGACCTATAAACTACCATTTATTGAGTAGCCTAGTAAGCTTG
TATGTTTCTTTTATTTATCATAACAACGTGTTGAGGTGTTGTTGC
TCCCATTTTACTGATGATCAGAGCCTCTGAGAAGTAAATAACTCA
GCCAAATTAGTACATGCCAGGCCTGGTTTCAGAGCCCCATTTGTC
TGATTCCAAAGCCTTTGCTCACAACCACAACACTCTCCTTAATTA
CAAAACAAGTGTCATGGGGCAGAAGACTGGAGTGGTCATTAGTTT
TGAAATCATCCTGTCCTAAATCTGATCTCACCATGATCTCCCTTT
TAGACTACATCAGGAGAAGATGTTCGAGACTTTGCCAAGGTACTA
AAAAACAAATTTCGAACCAAAAGGTATTTTGCGAAGCATCCCCGA
ATGGGCTACCTGCCAGTGCAGACTGTCTTAGAGGGGGACAACATG
GAAA

In some embodiments, the intronic domain that promotes trans-splicing primarily in the liver comprises or consists of sequences from the gene GJB1. In some embodiments, the sequences from gene GJB1 comprise or consist of:

(SEQ ID NO: 677)
TTGCCCAGGCTGGTCTCAAACTCCTGGCTTCAAGTGATCCTCCCG
CCTTGGCCAAAGTGCTGGGATTACAGGCATGAGCCACCATGCCCG
GCCCTGTCATACAGTTTTAGAGGCTGGGAGCTAGAAGGGTTGACC
AGGATGGCAGCCTGTACAGGATCCCTCTCTGCAGCTTGCCCGCAC
TGTGGATCTGCCTAAGGCCAGGGCATGGGGGAAGCCTGAGGGATG
CTGAGGAGCACAGGTCCAGCTCAGTCTCCCAGAGAGACTAAACAT
GGGTGGGTGACAATGATGCCTCTCTTCCTCTGGGACAAGGCAGTG
ACTCCAGGCTATGTGATCTGTCTCTCCAAACCCATAGGGAGCTCA
GAGGATCAATCTTGTTTAATGATTGTATGTGGGAGGAGGTGGGGC
TGGTTAATGTTTGTCTGGTTCCTCTTGGGCTGTGGGCCATTTCTG
GCTCTGGCCCTGGGTTCGTTGTCCCTCCCCTGGGCTGGGCTGACA
GTAC

In some embodiments, the intronic domain that promotes trans-splicing primarily in the liver comprises or consists of sequences from the gene GJB1. In some embodiments, the sequences from gene GJB1 comprise or consist of:

(SEQ ID NO: 678)
TGAGGAGACTGGGGGCTGAGGGAGGTGGGCCAGGGAGGGCTGATG
TGGCACAGTGGGCCGGAAATAGGGGCCGGGTCTTGCCTTCTACTG
ACCTCCGCCTTCAGCCACTTAGGTCCCTTCCATGCTTCCCTCTGA
AGCTGGGGTGAGGCCCCTCCTGCCAGGTGCCAGACTTGCTTAGCC
TCTCTCTGCAGCCTCTTTCCAGGTTCAACATTGCTTTTCCTCTCA
CTTTTTTTCCTCTGGCCAAAGATTCCCCTTATTCCCATAGGGCCA
GATTCTCAGGAAGACTTGAGGGACCCCCTAGGTGCAAGGTGGCCA
AGTTTGTCTCTCTGAGACATTTCCTGCCACACAGGGTTAGGGTTT
AGTGGGAATGTTGGGCACTGCCAGGTGCTGTTTTCCTTCCTTGAT
TACAAGGCAAGCCAGGCATGCACCCAACACGGGGCTGCGAGTGGA
TGAATAAAGCGTGCTCAGCGGGCTGCAGAGGGCTAGCCAACAGGG
CCAA

In some embodiments, the intronic domain that promotes trans-splicing primarily in the liver comprises or consists of sequences from the gene TXLNGY. In some embodiments, the sequences from gene TXLNGY comprise or consist of:

(SEQ ID NO: 679)
AGAAGAAATTGTCCTGGATCATACAATAAGCTAGGATTTGCTCCA
GACCATTTTTTTCATTTTATCAGGCTACCTACAATTAAGGAATTT
CAGAAGATTTTGTGAACAAAAAATGTCCAGGAATTATGTGTGTAA
TAAATGAATTTGATATTTTGATCTCCTTATCCATCTTTTTCTTGA
TAAAATATCAGCTTTTTTTTTTCCAATGTAGGGCAGAAGCAAGAA
GTACAGATATTTTGATGGGGACAGCCCACGTCTGATAGGGTGGGA
CATGGGGCAGCAGTTAGAGGGTTGTGCTCTTTCTAGTGTGGGATA
GTTTGCAAGATGATATGTTGTAGCCACTTCTGCAGTGACGGGGAC
AGAAGGAGTCAGTGTTCTTAACTACAGTGGTAGCAGCAGAAAAGA
AGGGTAAGCAGGGTTCTAGAAATTTGTGTTATGTTTTCTCCCCAC
TGTATTTATTTCTTTGGTTAGTGGTGCAAGAAATTCTGTTTTCCT
GTAG

In some embodiments, the intronic domain that promotes trans-splicing primarily in the liver comprises or consists of sequences from the gene TXLNGY. In some embodiments, the sequences from gene TXLNGY comprise or consist of:

(SEQ ID NO: 680)
TAAGTTCTACTTCTTGGAAAAAAAAAAAAGATCGCCAAAATTGTA
ACCAAAGCCATTTAAAACAAACTCCTCACTTAACATAGTGCCAGG
GCATGCAATTTTGTTTCTTACTCTCTGGATGTGGGATATGCGAGT
GTGTGTGTGTGTGTGTGTGTGTGTGTATTAAGCTTTCTGTTTCTG
ATGAAATTTTCATATGAAATTTACTGGAGATAAGATTAAATTAGT
GGAAAAACAAGATAATTTTGCTTTCTATGTAGTGGTCACCAAGTT
AAAGAATTGTGCCAGTTATTAGGCAAGATAGGCTTTGAGATTTAG
GGACAGTTAAATTTTACACCAACTTAGTGAACATGAGACTTCTAC
CTAGTGTTACATTTCATTTTTAAATAAGCAATTTAAAAATTGGTA
AATGATTTGTTTACTTTGATTATATTCTGGTATAATTTTCTGACA
AAATTATCTGTGTCTTGGTCAGCATTGTTGCTAGAATATGTATTC
AGAT

In some embodiments, the intronic domain that promotes trans-splicing primarily in the liver comprises or consists of sequences from the gene MICU1. In some embodiments, the sequences from gene MICU1 comprise or consist of:

(SEQ ID NO: 681)
GTACTTTTTAAATTGATCTTTGAAGGCTTGGGCTCTTTAATGGTA
GTTTATAAATTATTAAAATTTATTCATTAAAATATTACTCTCACA
TTTCTGGTGGCTGGCAGTCTTAAAAATATAAATTAAACTGAGTTG
TAGTAATTAAATATAAATTAAGCTGAATTATAAACATCTTTTGGT
TTAAAATAACTAATATAACTAACGAAGACCACAGGGTGCTCTTTA
TTTTTACAGTACATTTTTTCTATACTTCCTTTGTTTGAAAAATGG
GGTGTAATTTTTGGTAGTCCCTCTTACCAAACCCTCAAATTAGGT
ATCTTATTATTAGTACAATGGATTATCTTCCTAAATACCTTTGTC
TTTAACTGCATTCTGAACCAAACCAACAAATACATTAAAAATTAT
GTCAGAATAACGTATAGACGATTCCATTATCAATGTTTCTAATCC
TGGGAATTCTATTTTAGAACAGCCATGGCACAAACATCATACAGA
GTAC

In some embodiments, the intronic domain that promotes trans-splicing primarily in the liver comprises or consists of sequences from the gene MICU1. In some embodiments, the sequences from gene MICU1 comprise or consist of:

(SEQ ID NO: 682)
AGGTTAAGAAATTTTGTTGTAATCACTCTTGTTAAGCATAAGAGA
TACTTAATTAATTGAGTCACTTAAATAGTCTTACTTGGAGGCCGG
GTCTGGTGGCTCATGCTTGTAATCCCAGCACTTTGAGAGGCAGAG
GCAGGAGGATCACTTGAGCAACCGGGAGTTCAAGACCAGCCTGAG
CAACATAGCAAGACCTCATCTCTATGAAAACATTTAAAAGCAGTA
GTAGTCCTAGCTACTCAGGAGACTGACGTGGGAAGATCGCTTGAT
CCCAGGAGTTGGAGGTTGTAGTGAGCTAAGATTGTGACACTACTC
CAGCCTGGGTGACAGAGTGAGACCCTGTCTCTAAAACAAAAACAA
AAAAGTCTTATTTGGGATATTATGTTCCTACTTGAACTGTAACAG
GACTGATGAGACAACAGTAACTTCAAGTTAAAAATTAATCTTTTT
TTCTTTTGAAATTTCTTACATAATAAATACAAATAAGCAGTTGCT
TGGT

In some embodiments, the intronic domain that promotes trans-splicing primarily in the liver comprises or consists of sequences from the gene VDAC2. In some embodiments, the sequences from gene VDAC2 comprise or consist of:

(SEQ ID NO: 683)
GGCGGCCAAGCCGGAGGCCCGCGCCGGACTCGGAGTCGGCCCTGG
CTTCCTAAGATGGCCGCGCTGCCCCGCGGCCCCTCGCCCCTCTCT
GCCCGGGATCTCCCTTTGGCCCTTTGACCCCAGCTTACCGCACTT
CTTGTCCCTCCCGCAGATTCCCCTCTTCCCGCGGCCTCGCCATGG
CGACCCACGGACAGACTTGCGCGCGTCGTAAGTAAAGCTGGGATC
TCTGCGGGATGGGGCGGCGGAGAGTCGAAGCCTGTCGACCAGAAA
CCCAGTTTGTTTCCCCCTATCTTTCCAAGAACTTGGAAATTCGGC
TGCTTTTGGTGGTCGCCCCACCGTCTGACCACCTCCTCTGCGGAG
GAGGGGCTGGGCTGCTGGATTTCAAATGGGGAACAAGGCCCGGGG
AGTTGGTCTGTGGCCGCATGGACTTTTCAGCCTTTGTACATGTCT
TTGACGTATAGAAGTAAAAAGTTGTTAATTGGGGATTCTGCCTTT
GGAG

In some embodiments, the intronic domain that promotes trans-splicing primarily in the liver comprises or consists of sequences from the gene VDAC2. In some embodiments, the sequences from gene VDAC2 comprise or consist of:

(SEQ ID NO: 684)
TAAGTTTGAAGCGCTATTTGATATTTAGTTGATGTAGACATTTGA
GTGAAACACACATGGCAGTATTTTTTCCAAGTTTCAATATTTAAA
TTCCCAGTATTAAATTCTCTTCCCACTCCAGGGTGTTGGTTTTCC
CCTCAGCGAAGATATTAAAGGACGTATCAGGGTAAAGGGCCTAGT
CTTAGGTTTCGGTTTTATCTAATTTAGTTAAATCAGCTCATCTCA
GTGAATTTTGGCAGATACATAAATATCCATACACACAAATACTTG
TTTGATTCTTTGTACTTGTCCAGTCCTAGGAAGTGAGGCTGTAGG
TAATAGACTGTTTTTCCGTTTTGAAACGAATTCCCCAAGTTTGTA
TAATCATGGTTATCTGTTTTAGAAATAATTCGTTGGTGGTATCTC
CTTTATGGGTCGTTATAGTTCTATTGTAGCTCCTGACCGCATAAA
AAACATTCCAACAAATGGGATTGCTTGAATTTTAATATCAGCAGT
GGGT

In some embodiments, the intronic domain that promotes trans-splicing primarily in the liver comprises or consists of sequences from the gene CYP2C8. In some embodiments, the sequences from gene CYP2C8 comprise or consist of:

(SEQ ID NO: 685)
GTAAGTCTGCCTTATGTTCCTCCAGCCAATTGCAAAGGGTAAGTT
ATTTGACTGCTATTTTTAGACAAAATATATTCCTGGAAGCACACT
ATTATAATAGATCATTGTAAAGCAAAATACTCCCTCTGAACTTCT
TTGATGTTTCTTTTGTCTTCCTATTTTTTTTTTTTTTGAGACGGA
GTCTCGCTCTATTGCCCAGGCTGGAGTGCAGTGGCACTATCTCCA
CTCACTGCAAGCTCTGCCTCCCAGGTTCACACCATTCTCCTGCCT
CAGCCTCCCCCGAGTAACTGGGACTACAGGTGCCCTCCACCATGC
CCGGCTAATTTTTTGTATGTTTTAGTAGAGACAGGGTTTCACCGT
GTTAGCCAGAATGGTCTCAGTCTCCTGACCTCATGATCTGCCTGC
CTTGGCCTCCCAAAGTGCTGGGATTACATGTGTGAGCCACCGTAC
CTGGCCTCTTTTGTCTTTCTAAGTCTGTCATTGTCAGAAATAGCG
GAGT

In some embodiments, the intronic domain that promotes trans-splicing primarily in the liver comprises or consists of sequences from the gene CYP2C8. In some embodiments, the sequences from gene CYP2C8 comprise or consist of:

(SEQ ID NO: 686)
TATACATTTATTAGCTAATAATTTGATATATGTATGGTAATTCAA
CATGTATGAGTTATATTCACTATTTCATGTTTAGGCAGCTGTATT
TTAAGTGAACTATACTAAATATTTGAAAGGCTTTTGTTATCAAGG
GCTAAGTCTCCTATTTTTTGATATAGCATTACAATGTACATTTTT
TATACACAAAATATAGAATACACTGATTTCCCTCAAGGTCATAAA
TTCCCAACTGGTCATTAATCTGAGAATATTGAATTTTGAGTATAT
TCTAACATAGAATCATTTACTTCAGTGTTTCTCCATCATCACAGC
ACATTGGAACAACCAGGGACTTTTAATTAAAAATACCTGGGCTCC
AATCCAATACAATTAAACCAGAATCTCCTAGATTGGCACTGGAAA
GAAGGAGTAGGACAAAAGAACATTTTATTTCTATCCATGGGCCAA
AGTCCACTCAGAAAAAAAGTATAAATTGGATCTAGGTGATTGTTT
ACTT

In some embodiments, the intronic domain that promotes trans-splicing primarily in the liver comprises or consists of sequences from the gene GPAM. In some embodiments, the sequences from gene GPAM comprise or consist of:

(SEQ ID NO: 687)
GTGAGTGAAGACCCTTCAGTGTTGCCTGGTCTCTTTTCTTGAGCG
TTTTCATGAAAACGTAACCCCCCGTGTTTTTCTGTTAGATAATGT
TTGACCCATTGTTGATACTTTATATTCCACTCCTGGATTTCAGTG
ACCACCAAACCAATATGTCATTTACTATCTTGGTTTGCTCTCTGG
GTCACTGCTCCACTGAATCTATCCTGCAGCTATGAGCCATGATAG
GACAGATTGCTCAATGTTCTCAGGCAGTCACATCCAAAGCTAGTG
AGCACGTATTGTGTGTGAGGCACTGGGTCAGTGGTTTGTAAATAA
TTAACACACCCACCTGTAGATGATGGAACCACAGATGGTAAGAGT
CAGAAGCAGCCTTAAAAATCATCTGCTCGGCCAGGTGCGGTGGCT
CACGCCTGTAATCTCAGCACTTTGGGAGGCTGAGGCGGGCAGATC
ACGAGGTCAGGAGATCGAGACCATCCTGGCTAACGTGGAGAAACC
CCGT

In some embodiments, the intronic domain that promotes trans-splicing primarily in the liver comprises or consists of sequences from the gene GPAM. In some embodiments, the sequences from gene GPAM comprise or consist of:

(SEQ ID NO: 688)
GCCTAAAACACATAGTGGTATCTGCTTTAATTTGGGTGATAGCCC
CTTGCTTGAGGGAATGTGGCTATAATTTTAGTTTTCAAACGAAAA
TGTAATCTCCTGTTATATCACTGTTTACAATGAGTAGTTACTGAA
TTACCGTTTGTATAGCATTACCCTTATTTTTCAGGAGAGGTAGAG
TGTCTTGCTATCCAGGAGCTGAAAATCTAACTGAGATGAGACATA
CACAGAGCAGCAAAGTGAGTGGAGGGCCTCATTTGTGATTAAGAG
AAAAGAGAGGAAGGAGAGAAGAGGGGGCTTATGTCTTTGGAGAAG
GTTTTTGATGCAGTTGGGATCATATGGAATCATAAGAATTTAGTC
TTATGGGATTTACTTAAATTTCTCCAAATACAGGCCATTATTTTT
GATGTATTAATAATCTCTTAAGGTTATTCAAGCCAGTTGAAGTTT
TTCTTGGAGACTTTGAGCTGGCAACTTCATTGTTGCTTTTCTTTT
CTTA

In some embodiments, the intronic domain that promotes trans-splicing primarily in the liver comprises or consists of sequences from the gene ACSL5. In some embodiments, the sequences from gene ACSL5 comprise or consist of:

(SEQ ID NO: 689)
GCTACCTATTAGCTACATAAAGTATCTTTCTCTCCACCTCTTATT
TCCTACAGGTGACCCCAAAGGAGCCATGATAACCCATCAAAATAT
TGTTTCAAATGCTGCTGCCTTTCTCAAATGTGTGGAGGTCAGTGG
TCAGTTGAAAAAGAGGCTCTCATTAAAATGTGAATCTGTCATAAG
ATTTTTATTTTTGAGGTTAGAGCAGGCAGACACACACACACACAT
ACACACACACACACACACACGTTAAAGGCTGGAGTGTGGACAAAC
ACCAGCGTGTTCTTTGTTTAGAGGACTGTACATTTTCAAGCGTTG
GTCTTTTAGGATCACTGCAAATTTCTACACTATATGCAGTTCAAG
GCATCTCAGATCTACACAGTGTACCGCCTATACAGCTGTCTGTGG
TAGTCCTGGGAGGAGGTAGTTTTAAAATCTCCATTGGAATTGAAA
GTTGGCATTAATCTCATGTTGCCTCCTCTTACTTCCCTGGCCTAC
ATAG

In some embodiments, the intronic domain that promotes trans-splicing primarily in the liver comprises or consists of sequences from the gene ACSL5. In some embodiments, the sequences from gene ACSL5 comprise or consist of:

(SEQ ID NO: 690)
TGAGTGTTCTGTGTTCCTAGCAGTATGTGGCAAGGGGGTCCTGAC
TTGCTATCACATGTTTATTCCAAAGGCAGCTACACAGTATTTACT
GTTCTTTCTCCGGTGTGAGAGGTGCAGGCAAGGAGTTGGCTCATG
GGAGCCCTAATCATGGCATGGCCCCTCATTTCCTGCAGAGTCTTC
CTCTGCTAAGTCTGCATGTGTCCAGGGAGGATAGTACTTGCTCCT
GCCTTCATGGGGGTGTGTGAGTCTAGTGACATCACAGCTAGGAGC
TTACTATCTATCTGCCCTTAATTCTGAGCTGACACAAGACATTGT
CTTCTTTTTAAATTTTATTTTGGTTAGAATTTTTGAAAAGATCTA
GGATTAGAGAAAAGGAAGTTCCCTACAGAGGATCAGGGAAGGAGC
ATTTGATCTACATAAGGTGTGAGATGTCCCCCGCGCCGGTTTTTG
CACACCTGAGTGTGTAAGGTGGTGCTGTCAGCATGGTGCATACCG
GAGG

In some embodiments, the intronic domain that promotes trans-splicing primarily in the liver comprises or consists of sequences from the gene HABP2. In some embodiments, the sequences from gene HABP2 comprise or consist of:

(SEQ ID NO: 691)
TGAGGTTTAAAACAAAACTTTTGAAGCCAACAAGCAACTGTGGGC
AACACTTCAAAGGGCCCAGAATCAATGTTTGTCAATAGCAGTTTT
GCATCCTTTAATTTATGTCTCATGAATATAAAGTGTGATGTTATT
AGGAGCCCATCCATGCCTTAATTCTGTTTGAGATTTGATGAACCA
GAAGTCTATTAACATTCTGTACAAATCAGCACTGGCAGAGGCCTG
TGGCACAGGCTTCACCTGATCTTTTATCCTGAGGCTGGGATGCTA
GTCACGCTGTCAGGCTTCCTGCTCGGAACACCCAGCAGCTGTCAT
GTGCACCGTGGAGCCAGGGGGCTATGTCAGCTACTTTCTTTGGAG
AGTTTCTCCCCAGAAGAGAAACCACTTCATGACTCAGCCCAGCCC
CAAAGTTGCCATTTGTTTAATCAAGATAGTTAGTTTGCAAAACAT
TAAACATTAAACAGTATTTGATGCCTAGTTAATAATTTGCATCCT
CTTC

In some embodiments, the intronic domain that promotes trans-splicing primarily in the liver comprises or consists of sequences from the gene HABP2. In some embodiments, the sequences from gene HABP2 comprise or consist of:

(SEQ ID NO: 692)
TAAGTGTTCTTTTCTAATATTTGTAGTTGAGAGACTCCGAAGTTT
ACTAGGAGGACCAAGCTAGGCTGGGAGTGATGAGAAACTTTAGAG
TGAAAGGCTGGAGTTGCCAAGTTGGAGATGTTCTAACTACAAGAA
CCAATGCAGGCCTGTGGCTTGGTTTCTTTTGAGCAGCCACTCCCA
TGGGAACAGGTGTCTGACTTGAGCCATCTTGGAGCGGAGCCTCCA
TGAATGCATTCCAGCAAAGGAGACTGGGGCAAGAAAATGTGCCCC
AGGGCATGGATTGTAATCTGTCATGGATCTCATAGACCCCTTTGA
GAAGCCATGGAAAGCTATAAACTCTCTCCCCAGGAAAATATACCT
ATCTATTGGTGCTGACACACACAGTTTTCCAGCGATTTGTGGCTG
ACACTAGGGTAAGAAGCAATGTGCTATGGCCTTAGAGGGGAAGCC
TGCTGACTGCAAAAGATCCTGGCAGTGGGTCTGCATAAATAGTTT
GGAT

In some embodiments, the intronic domain that promotes trans-splicing primarily in the liver comprises or consists of sequences from the gene IGF2. In some embodiments, the sequences from gene IGF2 comprise or consist of:

(SEQ ID NO: 693)
GTAGGATTTGAGCTGTGTTTCCCGCCCTGATCCTCTCTCCTCTGG
CGGCCGGAGCCTCCGTAGGCTCCAAGCCTGGCCCAGATTCGGCCC
GGCGCAGCCGGCCTTCCGCGCGTCCCGCACCTGGCGGGGGCTCCG
GGGCTCCGGCGCGGCACCGGGGGGCGCTCGGGATCTGGCTGAGGC
TCCAAGCGCCGCGTGGCCGGCTCCTCCTGCTGGGGCAGGTGGCGG
CTGCGCGCCCCGCCCGAGCCCAGGGGCCCCCTCAGCCGCAACAAC
CAGCAAGGACCCCCCGACTCAGCCCCAAGCCACCTGCATCTGCAC
TCAGACGGGGCGCACCCGCAGTGCAGCCTCCTGGTGGGGCGCTGG
GAGCCCGCCTGCCCCTGCCTGCCCGGAGACCCCAGCTCACGAGCA
CAGGCCGCCCGGGCACCCCAGAAACCCGGGATGGGGCCCCTGAAT
TCTCTAGAACGGGCATTCAGCATGGCCTTGGCGCTCTGCGGCTCC
CTGC

In some embodiments, the intronic domain that promotes trans-splicing primarily in the liver comprises or consists of sequences from the gene IGF2. In some embodiments, the sequences from gene IGF2 comprise or consist of:

(SEQ ID NO: 694)
CCGCATCTGCTGCTGTCCCGCCGGTGCTGGCGCTCGTCTCCGGCT
GCCGCCGGGGAGGCCGGCGTGGGGCGCGGGACACGGCTGCGGACT
TGCGGCTGGCGGCTGCGCTCGCTCCTGCTGGGCGCCCCGAAATCC
GCGCCACTTTCGTTTGCTCATTGCAAAGATCTCATTTGTGGGGAA
AGCGGCTGGAGGGTCCCAAAGTGGGGCGGGCAGGGGGCTGGGGCG
AGGGACGCGGAGGAGAGGCGCTCCCGCCGGGCGGTAAAGTGCCTC
TAGCCCGCGGGCCTAGGACTCCGCCGGGAGGCGCGCGCGGAGCGC
GGGCGAAGTGATTGATGGCGGAGCGAGGGGGGCGAGGGGGGCCAG
GGGGGCGCGAGATTCCGCCGGCGGCCCCTTCCCCTTGGCTAGGCT
TAGGCGGCGGGGGGCTGGCGGGGTGCGGGATTTTGTGCGTGGTTT
TTGACTTGGTAAAAATCACAGTGCTTTCTTACATCGTTCAAACTC
TCCA

In some embodiments, the intronic domain that promotes trans-splicing primarily in the liver comprises or consists of sequences from the gene PSMA1. In some embodiments, the sequences from gene PSMA1 comprise or consist of:

(SEQ ID NO: 695)
GTGAGAATATCTTTGCTTTTTGATAAAACACAGAATGCATAGATT
TCACATCTCACTATGTGAGCCCCCACAAAACAAAACCTAGAGGAG
CATGTGAGAAAAACGCATCTCTAGGCTAGAAAACAAAAAGAGAAA
AAAAGAAAAATGTCTGTGTAAGAAAGCAAAGGAAAGAAGATGCTC
AAATCCAGCAAATCATACCAGCCTTGTCATAGGCACAAAAAGAAA
GGGATTAACCTAAGTTACAAGGTGAATTATCATAAAATCAGATGA
ACCTTTCTGAAAAATAGTCATGGGACAAAGGAGGGATATATAACA
CTAGAGGCTTAACCCTTTCCTGCAGAGAAAAAAAATGTAACTGAA
AGAAATGCCACTCAAGGCTTGTGACATTCAAAGTGAAGACAAGAT
AAGGTCTTATGTAGTACATGACATAGGTGTAGACACTCACTAGAC
TGAAAGGAGTACATGTTTCCAAAGCAGCTTTAATTCACGTCTCTG
CTGA

In some embodiments, the intronic domain that promotes trans-splicing primarily in the liver comprises or consists of sequences from the gene PSMA1. In some embodiments, the sequences from gene PSMA1 comprise or consist of:

(SEQ ID NO: 696)
TGACCTAATTGTATTCATTTGTCAAGAGGCCGGTTCCATATCGGA
GACAGGCTGTATTCATAGATGATTTTAAGGACAGTTTAAGGCATA
GAGTTTTACTCTAAAAACTGAGAAACAAAAAATCCTTCATTGTAG
AATTTGTTTTGTTGCCCTAGCTCTACTATTTATACTTTTCCACTT
TAGCTATGCACAGAACATAGCTAATTTTCATCTCAATTTTTCAAT
TCTTTTGCTATATAATTGTTAACATTTATTGACCACTAACCTCAT
TTTAAGGACTGTGCTAAGTATTTTATATACTTTCCTGGGGAGGCA
CTTATTCCTCACTTTTGATATTTCCCTCCACGTGGGGAACATCCA
TTAGAGTAACTCTCCTGGCCTGTATCTACTTACCCCCAGGAGAGG
AGAAGAGAGTTTGGTGAGTTTGAGATGAGGAACAGGATGTCTGAT
GAAGATCTAATATAACTCATGTTCCTTTTACCTTCTCCATTGTCT
TTCA

In some embodiments, the intronic domain that promotes trans-splicing primarily in the liver comprises or consists of sequences from the gene DEPDC7. In some embodiments, the sequences from gene DEPDC7 comprise or consist of:

(SEQ ID NO: 697)
TGCCTTTATGGTATTCACCATTCATTGTTAATTCCTAGTCTTGCT
TTTGCTTTTGGAAATAATTTTGAATAAGTGCACTTTTTAACTCAA
ACTGTAACTTGAGTTTTGTTCCCTTTAATATAGTCATATTTGGTC
AATTAAAAGCAGTTTTTTTCTTTTGGCTAAAGTGAATGGGAAGAC
GGAAATAGCTTTAGAAGCTACCCAGCTCCTTCTAAAGCTTTTAGA
TTTCCAAAATAGAGAAGAATTTAGAAGACTACTGTATTTCATGGC
TGTTGCAGCAAATCCTTCTGAGTTTAAATTACAGAAAGAAGTAAG
TCTTTTGTTTAACTGTTAGTTGTATTTAATATCCTTAATACTTAC
AGGTTAAGTATTAGATTATTCCATCTTCAGCATCAAGTATGCTGA
AATGTATAATAGCTATTAATATTTAATATTCCCTTTTATATTAAA
CTTGTCTCTTAAATATATACTATTGGATATATTTGTTTTATTTTT
ATAG

In some embodiments, the intronic domain that promotes trans-splicing primarily in the liver comprises or consists of sequences from the gene DEPDC7. In some embodiments, the sequences from gene DEPDC7 comprise or consist of:

(SEQ ID NO: 698)
TAAAATTGTGAAAGTAGTTAAATTGAGCTTATGTAATAGATCACT
AGATTAAAATGCATTTGAATAATGTCCCATGTCCCTTCTTTTTCT
TAAGATTCCTGGAACTCTACATAAAATTGTAAGTGTTAAGCTTAT
GGCCATACAGAACGGAAGAGATCCAAATAGAGATGCAGGTAATCT
GAGAAATATTATTTTCATGATCTTCCAAAGACAAATTTCAGGTGT
TTTTTGAAAATGTTTTGCTATAAGTAGTTGGACTTTAATTTCAAC
CAAATTTAAAACATTCTCCTCAGAGTTATTCATACAAAGCAACTT
TACAAGTGTTAGAAAGTCAGCAGTGCTTACCATCTTCAGTGCATA
TTACAAAATGGGGAAGAAAAACATAAAGACAAGAATATTGCTTGA
TTTTTCTGCTTAAATATGAGGAATTGAGTCATTAACTGAACTTTT
TACTTTTAAACTTTAAGGATATATTTATTGCCAGAGAATTGATCA
ACGT

In some embodiments, the intronic domain that promotes trans-splicing primarily in the liver comprises or consists of sequences from the gene CTNND1. In some embodiments, the sequences from gene CTNND1 comprise or consist of:

(SEQ ID NO: 699)
TGAGAGAGGAGACCACAATAGAACACTGGATCGATCGGGGGATCT
AGGCGACATGGAGCCATTGAAGGGAACAACACCCTTGATGGTAAA
TTCTCTTTATATACTGCTATCTGTCTAAGGCTAGTTCTATTTTGA
AGCCTATGTGTTTTGTGAAACACAAAAAAAAGTACAGAGAAAGAT
CGCATCCTTTTCTGGTAGGGGTTTTCAGGAAAAAGTAAGAGTTCT
GACTCATGTTGGGATTTCTTGGGCCGTTATTCTGCAGTGGTCAAA
ATGGGGGAAGCATGTCTGTAAAAGTGTTACTGATATGACTAACAC
TAACTGATCTACTTTCAAACATTACCTTTTTCCTCTCCCTCCCTG
TTTATGAATGTTTTGCCCTTCTCTTACTATTATATCTTGTTTTGT
TTGAGTTTACAAATTTTCAGAGTTTCCCTAGCTCTGGCACACACT
CATGAGGTTCCTGTCTCTACTCATACTAACAAATTGCATGTGTTC
ACAG

In some embodiments, the intronic domain that promotes trans-splicing primarily in the liver comprises or consists of sequences from the gene CTNND1. In some embodiments, the sequences from gene CTNND1 comprise or consist of:

(SEQ ID NO: 700)
TATGTCCTTCAGTCACCCCCAAGATTGTTCTTGAGGAAGGAGGCT
CCTGAGTGCCAGCAACTTGTCAATCACGCTAATCTGATCAGGCCA
GAGACTAAGTAGTCTCAGCCACATGGGGCTCGAGGGGTTCTGCTT
TTGTGATTATTACCTCTTACGAGTCTGGGACATGCAGCTGGTGAT
CTGATTCTCCGTTATGTGCTGCTTTCCTTTCTCCAGATTTGCTCA
ACTTTTTTGGGGGGGGTCTCCTTAATCCCAACCCCATTAACATTC
TCTCTTTCTCTTTTTTCTCCACAGCAGAAGATTTAGCACCACTAT
CTCCGTTCCATCTGGGCTTATATGTACTTTTATTTTTTGGTGGTG
AAATTGACTGATGATTTTCCTTTTTCTTCGCTGGACTATTGTGCC
AACTGCCAGGCTGCCTCCTGCCCTTACAGCCCTAAGTGGCTGCCT
TCTTTCCATCAACTCCCAACTTCTTCCTGTGAAGTTTAATTGTCT
CAAC

In some embodiments, the intronic domain that promotes trans-splicing primarily in the liver comprises or consists of sequences from the gene MS4A6A. In some embodiments, the sequences from gene MS4A6A comprise or consist of:

(SEQ ID NO: 701)
GTGAGCAAGGTTGGAGGTGATTCTGCCCCTCTCCCCAGGCTTTCTGTGT
GAGTCCATTCCTCCTCTCAGATTTATTGTTAGAACTTAAGACAAGCCAA
TTACATTTCATAATGTCTGTGTCATTCAGACGCTGAGAACTAATCCAGC
CTTCTAAATGGTGCTTCTTAACTCCTTTCTTCAACAGGAGAGTTAATGT
GTGGAATTAAGTGCAGAATCCATGGCCCTCTGTGGTGACGGTGATGGTT
CAGGTTGTGTTTCTGGGTTCATTCTGGAAGCTCCCCCAAGGAAAGGAGG
AAGGAAGCTTGCAGGGTGGGGCTTTGCCATTGCCCTGACTGGCTCTGGT
TTCCTTGCCTGATACATTGAAGTCAGCTTCCAAAGAATGCCACCCAAGG
TTTTGAAGGGGCACAGTGCCTCTGTCCTCACAAAACCAGCCTGCATATG
AGTTTACTGAATTCATTTAGGTCCTGCTGAATCTTTCCCATTTGTTCCT
TCTCTGTTT

In some embodiments, the intronic domain that promotes trans-splicing primarily in the liver comprises or consists of sequences from the gene MS4A6A. In some embodiments, the sequences from gene MS4A6A comprise or consist of:

(SEQ ID NO: 702)
TTCTGGTTGTTACACATAGGGTGGGATTGTGCTGGCATAGGCCAGGGAG
TCTGCTAAGCATCCTACAGTGCACAGGATAGCCACCTACTGTAAAGAAT
TACCCAGTCCAAAGTGGCATTAGTAGCTTTTTTGAGAAACCTGTCCCAG
ATCATTAATCAGAACGTGTTATTGATATACCAGTCTTCTCCCTTGTAGT
TAGGTTGGTAGAATTCAGACTCTGGAAAAAACTGGGAATGTAAAAGGCT
GAATGAGTGTAAGCTGAAAGACAAGAGAAAGGAGAATTGTTTTAAGCTT
ACTAAAGCCAAAATGGAAAAAAAAAGAGGGGGGAGGATTGGGGGGTGAG
GAAGACAGTCAACAGACCGCTTTTAGGCCATCATTCCGAAGGGTTCTGT
GCTAGGAACCTTACACGTGTTGTCTCACATAATCCTTAAAACTCAGAAA
TGATTTTAAATTAAATAGCACAGAGAGGAAAACTGACTGGAGAGGGAGC
CTTTGCTTA

In some embodiments, the intronic domain that promotes trans-splicing primarily in the liver comprises or consists of sequences from the gene SLCO2B1. In some embodiments, the sequences from gene SLCO2B1 comprise or consist of:

(SEQ ID NO: 703)
AAAATGGGGCTAATGTACACCTCGCTCATAGGAGTGATGTGGGGTTGCA
GGGCAGAGTTACTGAGCCCGTAGTAAGCGCTCCATAAATGCCAGCCACT
GGTGTTGTCTGGCAGGAGCTGGCGTTACAGTGGTTGGGGATGCTGGGGA
AGTGTCTGACTGTCCCAGCTCCAGGTCTTGCCAACTGTAGGAGTAAGGG
GAGAATTTGATGGGACCAGTGGGTGGAGGAAGAGGAGTGATGTCTGTGA
CCAGAAACATACAGAGGTCAGCACAGGACCCTGATGGTCAGTGGGTGTG
TTGTCTTGTATATGGGACCAGGCTCTGCAGGTTGAAAAAGGATGGGGTA
CGTCCCTGAGCCCCCAGCCTAGATCATCAGATGGGAGAGAGGCTGGCTC
TGGGTGGTGGGAGAGAAGGGCATAATAAGAACCCTTCAACGTTGATGCA
TGGCCCTTGGGGGCTGGAACCCCTCCAACCTCATGTTGCCCATGTCTCT
GCTTGTTAG

In some embodiments, the intronic domain that promotes trans-splicing primarily in the liver comprises or consists of sequences from the gene SLCO2B1. In some embodiments, the sequences from gene SLCO2B1 comprise or consist of:

(SEQ ID NO: 704)
TAAAGATCTTGCTTGGGACCATTGGTGGTGGTGATGGGGTCCAGATGCC
CACTGTGTGCCAGGCCTGGGGCACAGGCATGAGCGGAATTCAATCCCTG
ATCTTAGGGCTCACAAGCTCATGGGGTGACAGACCTGGCCCAGCTCTCC
TAGAGCGGGGTATAGAGGCAGAGTCTAGACAGCCCGTCAGGAAGCCACC
TCGGATGCAGGGGTGGGGCGGGCTTGAGGGCCAGACAGCCAAGAGGCCC
CAGTAACCCAGAGCCCCTGAGGGGTTGTACATGCCAGGGAGGGGGCAGG
GTAGGACGGTGGGCCTTCATTGTCCCCTGAGCACCACCTCCCTCAGCCT
GGATGCCCAGCCCCGTGATCCACGGCAGCGCCATCGACACCACCTGTGT
GCACTGGGCCCTGAGCTGTGGGCGTCGAGCTGTCTGTCGCTACTACAAT
AATGACCTGCTCCGAAACCGGTGAGACCTGGTTTGATGGCTTGTGGGAA
GGGTGCTGG

In some embodiments, the intronic domain that promotes trans-splicing primarily in the liver comprises or consists of sequences from the gene PICALM. In some embodiments, the sequences from gene PICALM comprise or consist of:

(SEQ ID NO: 705)
GTAAGTAAATAAGGAGAGCTTCAATAGTTCCATGGTAGAGAGAGGCCTG
CTGGAAGGTTCAGAAAATAGGCCGTTTGGAACCTTCTCCTTTAAGCCAG
GAGGGCGGCTTTGGGAGGCGTCTTGAAATACATTCTTGACTCTGCTGGG
AAAGCTCGGGGAGCCAGAGGCCTAATACCCCCCACTTCAGGGGAAGCTG
TGAAGGCAGCTGCACCTCCTTTGTCAAGCTGTTGGTTTCAACCACAGAC
ACGAGGACACACTTGAGGTGGGGGGATAAGGGGAGAAGCCTGCCGCCCC
GCCCCACCCCCGCCAGCACACCGCCCTCGCCATTCCTCTCCCGCCCCGC
CCCGCCCGGCGTCTGTGACCCCGCGGGCCGAGCCTCAGGCGACCTGTTG
GCAGGGAGAGTCACGTGATGCGCCGGCTGTGCACTCCCCTCGGGCGGGC
CTCCTGTTGCGCCCGCCTGGCGAGTTCCATCACGTAACGCGCGGGCGCC
GCCCAGCTC

In some embodiments, the intronic domain that promotes trans-splicing primarily in the liver comprises or consists of sequences from the gene PICALM. In some embodiments, the sequences from gene PICALM comprise or consist of:

(SEQ ID NO: 706)
TATTTTGGATTTGGCTGGCAGATGGTGTACACAGACTTTCTGAGAATGT
TAGCATTTCATTTGAAACTAGCAAGATTACATGTGACTATCATGGTAAC
TTAGCACTCACATATTTTTCAACTTATCCCTAACCCTGGGTTGAATGTA
GAGGCTCTAATAACATATAAAAGGCTTTGTTAATGTATGGTCTGGGTCT
CATTGAATAGTATTTCAGGATGTAATACTTCTTTTAAAATGTCCACAGG
GCAGTGTCAACGTCTTTCCATGGAAATCTGGCTCACCCAATAAGAGAAA
AGAGGCCAAAGGTAAGAAAGACTAAACCAGGGGAGATGGCAGGCAGTGA
TGTCTGGACTGTTTGAATGGGACTTCTCCATTTACCGCCCCTCTCACTT
CCTTCTATGGTCTGGACCTTTAATCACCACAGCAGTCAGCCATGTAGGG
TCAAGCTAATCGCTGTTGACATCATCCTACTACTTCCCTTTTCAAATTC
CAGTGAAGC

In some embodiments, the intronic domain that promotes trans-splicing primarily in the liver comprises or consists of sequences from the gene SLCO1B3. In some embodiments, the sequences from gene SLCO1B3 comprise or consist of:

(SEQ ID NO: 707)
TTTAGTATGTGACTTGCAAATTTTTCTTGACAGCCAGACAAAATGTATT
GGGTAAAAGGAATTGCTGTATATAAGCCTTTATTAATGTGGTGTAAGGC
GATGAAGACAAAGTAGCATCCTGTAGTTCTATGATAAGGCCTCAATCTT
TTAGTGAGCCTGTACCTCTGGACTGTGAACTTCAAACTTGCTTCTCAGT
TTCTGTCTCCCACTCCTTAGGTAGAATGAGATGGCTTGAGGGGGCTGGA
GTTATGTATTTTCCTCCCTCCCAAAGGAAGGCTAGAGACAGCTGGGGTT
TAGTTTCTGTAGCTTCATATGTTTTTTTAATAATAGAAAAATATCTTAT
GCCCCCAATGAAAATAATAAATAGAATTGTTCTCTTAATATTTGTATGT
GTAACTTGTTTTTCTTTCTTTTAAGATATGCATACTGGGGAGAAAAAAA
TGTAAGATATTTTACACATTTAAAATAATAATCGACTCTCTATTTTCTC
TTTTCACAG

In some embodiments, the intronic domain that promotes trans-splicing primarily in the liver comprises or consists of sequences from the gene SLCO1B3. In some embodiments, the sequences from gene SLCO1B3 comprise or consist of:

(SEQ ID NO: 708)
ACCCATTGATAAGATGTGTCTTCATTGCATCTGTCTGTTCTCTGGGAGA
AGCAAAATCTGTGTTACTTCCTTGCATACTTGTTTGTTTATTCATTGTG
CACAAGAAGATATCTTCCACATCTTCACAAGCTGGATAAGAAATAGAGA
AAATGAGATCAAATATTTAAATATTGCATTTTCTGGTAGTGATGCAGGA
TATTTCTTGGCCTCTTCACTGGACTCACAATAGAGGTGCCCTGTTTATT
TGGTCTGCCATGCTCAACCCCTTGTGGGAGGGAGAGTGTGAGTGACGTA
GTGCAGGACCATGTGGGCTGTCCTGGTCATCGACACAGAAACAAGCTCT
GTGAGTTTCTCATGGCCAGACCAGGTGCGAGTGAGTGAATGCTGGACCT
GGGCAACCGCTCCAGGCACTGAAACGAGTGAGTTACATGCAGGGCTGGC
AGCCAGGCAAGGTGCAAGGGTGCTCAGGATCCTGAAGCCCCAGAGGGGT
TGTTATGGT

In some embodiments, the intronic domain that promotes trans-splicing primarily in the liver comprises or consists of sequences from the gene ANO6. In some embodiments, the sequences from gene ANO6 comprise or consist of:

(SEQ ID NO: 709)
AATTACTGATGCCTGAGGCCACCTCCAGAGATCTGGTTTAATTATTGGA
ATGCAGCCTCCACTGGGATTTCTAAAAGCTCCCAGAAGATGTTATTGTG
CACCAGGGGTAGTGAACCACAGACCTGAGATGTCAGGGTCCTGAGAGCT
CATCTCATCCAGTGGTTCTCAGCCCTGGCTGCAGATTAGCATCATCTGG
AGAGCTCATGAAAGAAGCACTGAGGTCCCACCACCTGAAACGACCTTTC
AGAGGAAGGAGCCATTCTTTTTAGAAGGTGCTCAGGTGATTTTGATGCA
CAGCAAGAGTTAAGGTTAACTGACATCTTAGCCATCCCATGTTATAGAT
AAGGAAAATGAGAGCAAAAATGGTCAAATGGCTTGTACGAGGTCAGGTC
CTTGTTGTTACAGAGGCAGGTCTGATGCCGCCTCCTGCTCTGTCTGCAG
CCTAACACCATCCCTTATATTGGCCTGTTGTATTTATTGATGCCTCCTC
ATCCTTCAG

In some embodiments, the intronic domain that promotes trans-splicing primarily in the liver comprises or consists of sequences from the gene ANO6. In some embodiments, the sequences from gene ANO6 comprise or consist of:

(SEQ ID NO: 710)
TAAGATCCCCTTCCTTTGTATTTGAACAGCATCCTTCCTTCTTTTTCCC
CTCCGACTGTCAGTTCCGGGAGAACAGGCCTGTCTTTCCTGTTATTTCT
GTAACAGTAGTGGGGTTTCAGATAATATTTGTTGAATTGAAGTTCTGTA
ACTTTTTATTCCTCAGTCCACATGGATTGCTGAAAATAATCCATTCCTA
TATGCATGCTTCAGAAAAGAACTTCAGAAAGCAAAAGTTATTTTTTTTT
TGAGACAGTCTCACTCCGTCACCCATGCTGGAGTGCAGTGGTGCAATCT
CAGCTCACTGCAACCTCTGCTTCCCAAGTAGCTGGAATTACAGGTGCAT
GCCACCACACCCAGCTAATTTTTGTATTTTTAGTAGAGACAGGGTTTTG
CTATGTTGACCTGGCTGGTCTTGAACTCCTGACCTCAAGTGATCCACCC
ACCTCGGCCTCCCAAAGTGTGGGGATTACAGGTGTGGGCCACCATGCCC
GGCTGCAAA

In some embodiments, the intronic domain that promotes trans-splicing primarily in the liver comprises or consists of sequences from the gene KRT8. In some embodiments, the sequences from gene KRT8 comprise or consist of:

(SEQ ID NO: 711)
GTGGGTGTGGGTACCTCTGACCGGACCTGCTTCCCTATCCCTGGGACCT
GGGGTGGGGACGGTGGGAGCCCCCTGAAGCCCCTTGGGACTTGGGGTCC
TGTTGTTCTGGGCCAAGAAGGGCTAGGAGTTGGTCCTGACACCCCATTT
GTCTGGGTACAGGCTGGAGTCTGGGATGCAGAACATGAGTATTCATACG
AAGACCACCAGCGGCTATGCAGGTGGGTGTCCCAGGGCCCTGGGATGAG
GGCGGGAGGCAGGGCCCAGGGAGGCCTCAGCTCCAGGGAGGGGGCTGTG
CTCAGTCGCTCACAGTGACCTCAGCCTGAGCACTCATGTTCTTGGGAGA
ATCCTAGGGTGGGGAGGCACATATTCAGGACCCCAGTAATAACTTTATT
ACTTAGTAACTTTACTATTAGAAAGCACCCAAATAAACCATAGCTGTGT
GCCAGGCACTGTGCTAAGTATCCTACAGGTTTTATGTGATTTATTTTAT
TTATTAATT

In some embodiments, the intronic domain that promotes trans-splicing primarily in the liver comprises or consists of sequences from the gene KRT8. In some embodiments, the sequences from gene KRT8 comprise or consist of:

(SEQ ID NO: 712)
AAGGGTGTGGAGGGGAAGCACCTGGAAACTGCTCATGTCTCCCTG
ATCTGCTTCCTTAGTCTCGTTTATTTATTTATTTATTTTTGAGAC
AGAGTCTTGCTCTGTCGCCCAGGCTGGAGTGCAGTGGCGTGATCT
CGGCTCACTGCAAGCTCCGCCTCCTGGGTTCACACTATTCTCCTG
ACTCAGCCTCCTGAGTAGCTGGGACTACAGGCGCCCGCCACCAGG
CTGGCTAATTTTTTTTGTATTTTTACTAGAGACGGGGTTTCACTG
TGTTAGCCAGGATGGCCTCGATCTCCTGACCTTGTGATCTGCCCG
CCTCGGCCTCCCAAAGTGCTGGGATTACAGGCATGAGCCACTGTG
CCCGGCCCTTAGTCTCATTTAATTTGAGCTGGGGGGAGTCAGCCT
AGGTGGTGGTGGGGAGGACCCTGAGGGAGGGTGGGAGCACGGGAG
AAGAGAAGGCATACCCAACCTGACCTACTTACCTGTCCCCTACCC
CACA

In some embodiments, the intronic domain that promotes trans-splicing primarily in the liver comprises or consists of sequences from the gene CSAD. In some embodiments, the sequences from gene CSAD comprise or consist of:

(SEQ ID NO: 713)
GTGCATACAGTCCCCTTCTTCCCAAATCCCGCCCTTCAAACCATT
GGTCCAGAGAATCATCATGGACTAGGGAGTTTAACATGGCTGGGG
TAGGAGGCTGAGGGGAGGGGGACTGGAGAGAGGATTGATTGGAGC
TGGGAGGTCAGACATGGCAGTTCTTCCTGATGCTGGGGTGAGGGT
GAGACCAAAGCCCCTTTGCTGGGGACCTGGAAAAGGAGGAAGGGC
TCTGGTTGGGCAGCTGGAGGAGGAGCCACTGACCTGCCTTTGCTC
CACCCCATCCAGGGCTGACTCTGTGGCCTGGAATCCCCACAAGCT
CCTCGCAGCAGGCCTGCAATGCTCTGCACTTCTTCTCCAGGATAC
CTCGGTGGGTCTGCCCCTGCCCCTGCACCAGCCCCACCTCTCTCT
GGAGCTCTGCCTTCTCTATCCCACCTTAGATAAAGTTCAGGCCTT
TCTCTATTCTCTTTACCATCCCTTCTGAAGCTGCATCTCAGCTAC
CACC

In some embodiments, the intronic domain that promotes trans-splicing primarily in the liver comprises or consists of sequences from the gene CSAD. In some embodiments, the sequences from gene CSAD comprise or consist of:

(SEQ ID NO: 714)
CACTATGCCTGACTCAATTTTTTTTCAATAGCATTGGATCTTCCA
GTAAAACATTCAAGTTCTTTGAGAGTATGAGCTTTGGAAATGAAA
ACCTAGGTTCGAAGTCCATCTCTGCAGCTTCTGCTTGGATCTTTG
AATCTCAGTGTCCCTGGCTGCACATTAAAAAGGACAGTAACACAT
TGTTGTGAGGTTGAAATGGGCCATTTTAGGCATGCAAAGAGCTTA
GAGCTGGGCCCAGCATCAGGCTCTGAGTAGCTTTTGAGATGACTT
TACATCTTTCTCCCTCTACATTTTGGCAACAGGGTGCTGTGCCGT
TCCTGGTCAGTGCCACCTCTGGCACCACTGTGCTAGGGGCCTTTG
ACCCCCTGGAGGCAATTGCTGATGTGTGCCAGCGTCATGGGCTAT
GGCTGCATGTGGATGTGAGTGGGACTTCGGCTTGGGGGTGCGGTG
GGGTGGGCTGAGCCAAGGCCAAGGCCCACGTTGTTCCTCCTGCTC
CACA

In some embodiments, the intronic domain that promotes trans-splicing primarily in the liver comprises or consists of sequences from the gene RDH16. In some embodiments, the sequences from gene RDH16 comprise or consist of:

(SEQ ID NO: 715)
GTAACCACCCAGGTTTGTCAAGTCTGTCTGTGTCTTCCCTCTGTG
TGAAGGGACCACCTCCTGTCTGCTCCCTGGGAAGATTCCTACAGT
GTCTTTCCAGGTTGAAGTAACAGTAGCTTTCTAATATTTCTCACT
TAACAGGCTGCCTGAACCCCATCTCTTTTCAATCCTTAACCTCTC
CCGACATTGGAGGTGTCTAGTCTTGGGGAGAGTTTGCCCTGATGA
GATTTTCTCTCCAGAATTCCTCTCTATCCTCTCCCTCCTGTCTAA
GCAGAAGTCATGTGTTGTTTGATGCTAAGAGCCTGGGTTAGAGAT
CTGGAGACTTGCTGGAGCATCGTTGAGAAGGGAGCACAGGGCTGC
AAGTGTGCACGGAGGCTGTGCCTGTGCTGGAGCCAGGCTTATGTT
CCCAGCACTTTACCTTGTTTTTGCTAGGGCCCATCAGCGTCCACT
GAGAGCTACGATCATCTCCATCTTCAGAGGAGAGTATCAGCTTAA
GGAC

In some embodiments, the intronic domain that promotes trans-splicing primarily in the liver comprises or consists of sequences from the gene RDH16. In some embodiments, the sequences from gene RDH16 comprise or consist of:

(SEQ ID NO: 716)
TTCTCATCCTAGACAGCCTGACCGTGCCCTAGTTTTCAGAGGAGT
GATTGATTCTGGGGTGCGGAGCAGGATGAGGAAACTCAGGCTCAG
AGAGGGATTTCCACCTAATGTGCCTGGAAACCATGGGTAGCTACC
CAATAAGGCCTTTCTATGTTCAGAAGGTCATGGACCAGGAGTGTT
GGTATCGGAGCTTTGTAGAGACAGTTTTGACCCCTGTACTTAGAG
CAGAGGGGTGTGTTTGCCATGGAGAGTGTGTGTGGGCAGCTGTGT
GTAAGCCTGTGTGAGTTCTGTGTATGTTCCTGTGTGAATATATGT
GTGTGCTCCTTCATGGACACAGCATGCATGTAGTGGGCATAGCAA
GGCAGAGAGAAATCAGGAGACTAGGGGCACATTGCCCAGTAGGAA
GATAGTATCTTCCTACTGGGCAGGACTTGAGGTGTGCAAAGGTGT
GCATGAGTACTGCAAAAGCAGTTCAAAGAGCAGAGAGGTGGGGCC
AGAA

In some embodiments, the intronic domain that promotes trans-splicing primarily in the liver comprises or consists of sequences from the gene MON2. In some embodiments, the sequences from gene MON2 comprise or consist of:

(SEQ ID NO: 717)
AAGCAGTCTACCTTACTTTTTTTTTTTTACTATATTGTTCTGAAG
TCAGGTTATTGCAAGAATAATCCATATTTCATATATTTTTAATGT
AGAAGATAATTGACTTATTTGGGGACAGGTGGAAAAGGGCTTTAT
CATCTATTCGTACTGATTTACTTTGCAGATACAAACTGTATAAAT
AACTTTGAATCATTTTTACCATTCTTTCTCATTTGTTTGCCCTTT
CTGTTGTTCATTCCTGTCCAGTCCATTCTTTACATGATAGCCGGA
AATGATTTTTCCATAGTAGGGCTTTGATCATTTATTTCCTCATCC
TCCCAAAGCAAAAAAATATTTTTCATTTACCGTTTCCTACCAAAT
TGCAAAGAATACTCCAGTCTGACTCCATTCTAGTCACACTTTATT
TCCCACAATAGATTTCATTTCCCCTACTCTGCACTAAACTAAAAT
TCTCAATCTTCCCAAATTTTTTCAACCTTTTGTTTATTTCATTAT
CCAG

In some embodiments, the intronic domain that promotes trans-splicing primarily in the liver comprises or consists of sequences from the gene MON2. In some embodiments, the sequences from gene MON2 comprise or consist of:

(SEQ ID NO: 718)
TAAATAATTTTTCTTTTCATTGAATGCAAAGCTTTTTTATGTGTA
CTTGTCTCGAAGCACTCTATTTGTATTACAATTATGCATGTACAT
GACTCATCTCCCTTTACATTGTTAAGATTTTTGAGGGGCTTTTCA
GACCATGTTTTGTTCTATTTTGAATCCTCTACAACATTGCTTTTC
AAAGTATGGTTTGTTGGACCAGCCATTGATTGGCTTCAACAGTTT
TGTTACTGGTCTGGGACAAGATAGTGAGCTGGCATCAGAATATAA
ATCAGATGTGTCACTAAACACACTGCTTGGTTCAGCTGACTTTTT
TTTATACAAGAAGACATTGGTAAAGAAGAAAACTGTGTGTTGGTT
TACATTCTGGCACAAGCTTCTTATTTTGTAGCAGATTGGCACCTT
AAGGAGCATTACTTTTAATATCCAAGAAAAAAGTAGATTCCTAGT
AAATTACTAATGAATGAGTTGTAGGATATTTGTTCCTGTCAACTA
TTAT

In some embodiments, the intronic domain that promotes trans-splicing primarily in the liver comprises or consists of sequences from the gene BTG1. In some embodiments, the sequences from gene BTG1 comprise or consist of:

(SEQ ID NO: 719)
CTTCTGACTGTTATTTATGAGCCTTTTCATGGTATTTATCATTTT
AGTATTGAAGATGCAGTGTTCTTTTGGGAATTACGTATGCTTAGA
AGAGAGTACTGATGCTCAAAGGTGAAATGATTTGAATCTAGAGTT
GGATAAAAAGGGAAAGGATGGGGAAAATGAAAACTGGGAAGCTAA
ACTGAGAAGGAATTGCAAGGTTTGGTGATAAAAATTCATTTGCTA
TGGTCATAGTTGTGTATCGAGAGGTTTGCACAGCACAGTCTCTTG
GCAGAGTTTAGGAATCTCTACTCTGGGGATTCTGCAGTTCATGAA
TCGTCAGGGAGACTCTGCATTCAATTTGTTTCTGTGAACATGCAG
GTGAAAGTGGTAGTACAGGTGGAACTTCATGGGAACTGGTTTTGG
GGGAGGTGTGGATGGAAAGCACAAATGTCTCAGTATTTTCTAAAG
GGAGACTATTTTTAAAGGTGTCTTGTTACAAACCACTTGATTATA
GCCT

In some embodiments, the intronic domain that promotes trans-splicing primarily in the liver comprises or consists of sequences from the gene BTG1. In some embodiments, the sequences from gene BTG1 comprise or consist of:

(SEQ ID NO: 720)
CACACCATTCTCGAGAGTTAGAATTCGTAGACCCGCAACACATGG
ACTACCCTTGCCAATTGTTATGGTTTTATTTTTAAACCTATTCTG
TGAAAAAGCTCCTCGGTATTTCAAACTTGAATTACAATAAAAACA
CCAGGTAGGGTTGTTTTTAAATGGGTCTCACCCCAGTGCTGACTA
CTGTTGTTTACCAACTACATTTCATCTGGTAACTCCATTGAAACT
AAGTACCTCAACCATTTTTTTTTTTTATCATAAAGCCTTTTTTCA
AATGGAAAGTTGTTTTTGAAGATCTTGACATCAGACTTTTTCAAT
CTTTGAAAATGATGGAAACAGTTAATAAAAATGTTTATTTTAGAA
TTCAGGTGATGTCTGGCTTGTGAGGAGCTGTTTATAAGTTTGTCT
TTGAAAAAGGCTTGCACACATCACTCTGAGACCAGCTGTTTCCTG
TGACTTTAACCTTACCTTCCCCAGACAATTGAGCTGAACCTGTAT
CTGC

In some embodiments, the intronic domain that promotes trans-splicing primarily in the liver comprises or consists of sequences from the gene HAL. In some embodiments, the sequences from gene HAL comprise or consist of:

(SEQ ID NO: 721)
TTATGGTGTCGCTTTATTCTTAGGTATTTATAGCAAAGTTAAGCG
CCTCTAACAGCAGTTAATCAAGCTTCTGAAGGATTCAGTTGACAG
ATGTCAACCGTTTTAAGAACAATATGCTTGACTCAGTACACTCTT
TTTCCCATTGGCTAGCAACAAAAGAGTTGGAGGTATCAGGATTTC
CTCCCCAGCCCCGCAGGCTCACCTGGTCTTTGTCACAAGTTGTCT
CAGGGCAGCTGGAGGAGGCAAACCCTAAAGAACAAGGGCTTTTCA
AGTCCCTGTTGTGCCACGTTCGCTAATGTCCTGTTGGCTAAAGCG
AGTCACGTTCCAGGCCCAGAGTCAATTTGGGAGGGTAACACTCAA
GGGCATGGGCATGTGATTTGACGTGATCCATGGGCCTGTATCTGA
CAATCTACCACATGCATAAAATCCCATCACAGGCTGGGTGCAGTG
GCTCATGCCTGTAATCCCAGCACTTTGGGAGGCCAAGGTGAGTGG
ATCA

In some embodiments, the intronic domain that promotes trans-splicing primarily in the liver comprises or consists of sequences from the gene HAL. In some embodiments, the sequences from gene HAL comprise or consist of:

(SEQ ID NO: 722)
AGCTACTCAGGAGGCTGAGGCACAAGAATTGCTTGAACCTGGAAG
GTGGAGGTTGCAGTGAGCTGAGATCATGCTACTGCACTCCAGCCT
GGGCGACAGAGTGAGACTCTGTCTCACAAAAAAAGAAAAAAAACA
AAAAAACAAGAAAAAAACCACATAGGTACTGTTAAGACCCTGGAG
CAACTATCCTTCACTCTATCCACACTGAATTCTCTGGGTTTTGTT
ATTTGCTTGCTTGTTTTGCTTAGGAAAAGTATGCAGCTTAAAATC
TTTATGATGACTTTAGAAAGTTTTGGACTTTGTATATTTACAGTC
TTTAATGCAACCAAAGACTAATTGACACAGGTCATGTGTCAAATG
GCTTAAAATAAAAGCAAGCAAACAGCAGCTGTGTCTGGTCATGTA
TAGAGTATGTTTTGACTTCGGATGAAGTTGATGTTTAATAGCATG
TGGAAACAATGTCGTCAATATTTCAACTTTGTTTTGTCCTTGCTT
TTCA

In some embodiments, the intronic domain that promotes trans-splicing primarily in the liver comprises or consists of sequences from the gene NR1H4. In some embodiments, the sequences from gene NR1H4 comprise or consist of:

(SEQ ID NO: 723)
ATTTGGGTGTCGAATGATGCCATGCTCATGATTTGTAGAGCATAA
TAACAATGTTAGCCTTAGGCCATTTTTATACATTATCTTGAGGCA
GAAGCTAGTTGTTACTTTGCTTTACCATAAAAATTATGACTTGTG
ACTAAAAAACTATTCAGCAGAACCTTCTAAAGTTATGTATGGATA
TGTCCTATGACCCAGTGATTTCACTTCTGAGAATATACTCAACAG
AAAGGATGCTCACGTCCACCCTACAGGTTATCTTTAGGGGTTTTG
ATTGGAAGGGGAATTGGGAGGCTTCTGGGAAGCATTACATGTGTT
GTATCTTGATCTCGTTATAATTACGTGTGTGTGCATAAAAATTCA
TCAGGTTGAACACTTAATTTTCACATTTTGTGTACAACATTTAAC
TTACACTTCAAAATAGTTAACGTTGCTATAATTATGCTGAATTAA
TGCTTTTCCACTTTTTAATTTTGTCATTTTATTTTAAACTTCAAA
ACAG

In some embodiments, the intronic domain that promotes trans-splicing primarily in the liver comprises or consists of sequences from the gene NR1H4. In some embodiments, the sequences from gene NR1H4 comprise or consist of:

(SEQ ID NO: 724)
TTTCTTTGGGAACAAAATTTGATAAATATACAAACTCATTTCTTT
AAATGTTGAAAGACATCCTATTGAATCTGAGGGGGTAATTTACAG
AAAGATACTGTCTCTCGCACTTTTGTCATCTCTGGAAAAGTCCTG
GTGAATAGTCATTCATTTGTAATAGCCCTCTGATCATTTCCTTTT
CTTTGAAGTTCAAAGAGAATAGCAAACAGAAAAGTTAACAATTTT
CACATAAAATATTCCCTCAAGCAATCCTTTCCCCCATAGTTAGTC
TTGAGGCAGGATTCACAAATTCAGAGTATGCAGCTAACTGCAGTG
ATGGGACCTTCGCTTTCCTCTCTTAAAAGTGAGGAAATGATTGGC
TTCATTGTATCCAATCATCTTTCTGTTTTGTTTTGCTTTTGAAAA
CATTCCTACAGCTGGGAGCACAGCTGCTGTATACTTCCCCCAAAG
CAGGAATTAGTTCATTTCCTTCCTTCATTGTTACACTGTTGCATT
CTTC

In some embodiments, the intronic domain that promotes trans-splicing primarily in the liver comprises or consists of sequences from the gene IGF1. In some embodiments, the sequences from gene IGF1 comprise or consist of:

(SEQ ID NO: 725)
ACCTTTTAGAGAATCTCGCTAAGAAACATGGAGAAAACGGAAAAG
AAAAATGTAATGCCCTAGAAAGCGCAAAGAAAGACAGTGGCAAAA
ATGAAAAAAAAAAATAAAAATTATAAAAGAGGCAAAAAAAGACAC
ACTATTCTCTGCCTCTAAAACACAATTAAATAAAAGAATTTAAAT
AAAAATTAAGGCTTCTATATGCATTTTTAAATTTTGTATGAATCT
GTTATGGAAGAATTGCCTATGTCAATATATGTTCAGAGTTAAATA
TTAGCCCCAAATGCTCAGCAAGACTGAATTGTGTCATAGAAGTTC
CCAGATTCCCTTTTCCCGCAATGTCATTGGAGGCTGCATTTCTTA
GTCAAGTCCAGGGTTTAGGCCAAAGGGCATCCGGTATTGCCTAAA
ACCCTGTGAGGTCTGTGAGGTAACTTTTGAGAAGAGGTCACTGCA
CTCTTCATCTTTTTTGCACTTTGGAATCAGATATAAAAGATGTAT
AAGT

In some embodiments, the intronic domain that promotes trans-splicing primarily in the liver comprises or consists of sequences from the gene IGF1. In some embodiments, the sequences from gene IGF1 comprise or consist of:

(SEQ ID NO: 726)
TGGTCTTAAACCTCAGCTGAGGCTGGAAGAGGACCATGGTGGCTT
ATTGGAGATGTGACCCCAGGGAGCCCCTCTGAAGGATGGAAGGGG
ACTGGGCAAGACCCAACACACACAGAACACAGTAGCCACTGGCCA
GGCAGGAAGCAAGGATCTCAGAAAAGACTTTTAGGTGAATGTGGC
AGGAAAGCGTGCTTGCTGGGGCAAAGGCAGATTCATTCTTTCTCT
TCCCAGGTGACCCAGCGCCTCTTGGTTTCTAACTGGGGAGGGGGT
AGGTGTCAAGAGATGAGTCCCAAAGTTCTGGAATGGTGGGTCTTG
TGACTGAGGTCTAGACCCCTCTCCAGCATGAGTGCTGTCTCCTGC
ATCATATGGAGCCTGGGCATTCTGAGCTCATTCAAAGGGACACCA
TGGGAACCACTTGTTCTCAATGCAATTATTTTTGTGATGTTTACA
GTATCAGCCCCCATCTACCAACAAGAACACGAAGTCTCAGAGAAG
GAAA

In some embodiments, the intronic domain that promotes trans-splicing primarily in the liver comprises or consists of sequences from the gene HPD. In some embodiments, the sequences from gene HPD comprise or consist of:

(SEQ ID NO: 727)
TACTGTGTCTGTGTTTGTGTTGCGCATAAGGCAGTAGTGCCTTAT
GGCAGCCAGGGGGAGGCCAGGGCGGCCGCCAGCGCCGCGGGACCG
AGGAGCTGCGGGTGCCAGCGCTTTGCCAGCGGGACCACGCGGAGA
GGGGAATTCTGGAACCAGGGAAGGAAGGGGACTTGCTCTGGAGGG
AAAGACGGATGACACTGCTTTTGTAGAGAGATAAACGCGATTCAG
ACAATAAAGCAAGCGATGAGCCGGGCGTGGTGGCTCACACCTGTA
ATTTCAGCGCTTTGGGAGGCAGAGGTGGGAGGATCGCTTGAGGCC
AGGAGTTCGAGCCCAGCCCGGTAACATAGCAAGACCCCGACCCCC
CTGCGTCTACACAAATAACAAATAAAAATTAGCCTGGCGTAGTGG
CGCACACCTGTGGTCCCAGCTACTCAGGAGGCTGAGGTGGGAGGA
TCGATTGATGGACAGAGGTCTAAGCTGCAGCGAGCCCTGATCCTG
ACAC

In some embodiments, the intronic domain that promotes trans-splicing primarily in the liver comprises or consists of sequences from the gene HPD. In some embodiments, the sequences from gene HPD comprise or consist of:

(SEQ ID NO: 728)
ACAGCGCAAGGCTCCGTCTCAAAAAAGAAAAAGAAAGTGTCCTGA
TAATGTCTGCGGGGAGGATGGATTGTAAGGATACAAGATTAGAAG
CGGGCAGCAGTCTTGAATGGTGCCTTAGGAACTGTAGTAGGGGGA
TAGTAATAGGACAGTTTGGGGATATATTTGGAGGTGGCTGAACTG
CCAGAAAAAGAGGTCATTTCCAGACTTTTGGGGTGCAGGAGCCCC
TGTGGACTTTCCAAGGATCTCACCGTGCCAGCCTCCCCTAGCAGA
GCCCCCTGTCTCCCGCCTAGGAGCTGAAAATCCTGGTGGACTACG
ACGAGAAAGGCTACCTCCTGCAGATCTTCACCAAACCGGTGCAGG
ACCGGCCCACGCTCTTCCTGGAAGTCATCCAGCGCCACAACCACC
AGGTACTGCTTGTCCCCGGCAGGCCCGAGGGGACAGGCAGCTAGC
ACACTCGGTTTTCTGGGTCCTTGGCTCATCTCCTCTCTTTCCCGC
CACA

In some embodiments, the intronic domain that promotes trans-splicing primarily in the liver comprises or consists of sequences from the gene PHF11. In some embodiments, the sequences from gene PHF11 comprise or consist of:

(SEQ ID NO: 729)
CTTCTCCAGTCGTTCCAGTCCTCCCCGGCCCTCTACTTGAGTGGG
TGGGTCTTCTGATGGGTTCCTGCCCTTGCGTGTGTGTATAGCTCC
AAACGGTGTGCCAGGCTCAGGGCAAGGTCCTGAGCATACAAAGAT
GAGTTAGGCAGACCCTGCCCTAGAGACTCTACTAGGCAACTTTAA
TGAGGAGGTCAGAACCACAGAGGTACAGATGAGAGACTCCTGACT
CCCAGTCTTCAGGGAAAGCTTTTTGAGGAGAGGATGGCTGGGCTT
ACCCTTTTTTTTTTTTTTTTTTTTTGAGAGGAGTTTCACTCTTGT
TGCCCAGGCTGGAGTGCAAAGGCGCTATCTCGGCCCACTGCAACC
TCTGCCTCCCGGGTTCAAGCGATTCTCCTGCCTCAGCCTCCCGAG
TGGATGGGATTACAGGCATGTGCCACCACGCCCAGCTAATTTTGT
ATTTTTAGTAGAGAGGGTTTCTCCATGTTTCATGGGCTTACCTTT
AAAG

In some embodiments, the intronic domain that promotes trans-splicing primarily in the liver comprises or consists of sequences from the gene PHF11. In some embodiments, the sequences from gene PHF11 comprise or consist of:

(SEQ ID NO: 730)
TAAGTCACGGGAGATGCGCTTCCATCCTGACCTCTTCCTCAAACT
CCAGACTGGTATAACCAACTTCTTCCATTTCTGAAATGATCTCAA
ACTGAGTATGTCCCAAAGAGAATTCTTGTTTCCTCCACTGTATTC
AGGTACTCCAAACCTGCTCAACCACCAGCCCATTCCATTCGCCCA
GGTGCTCCTTGCAAAAACCTTAGTGGCTCCTCAGGGCCTCTCTTT
CCTCCGGACACCCCTTCTAATGCATCAGTCGGGCCAGCCAGTTCT
GTGTTCAGCCTGACTGTATCTGGAGCCTGACTGCTTCTCACCACT
TCCATTACTGCCACCCAAGCCTACGCCATCATGATCTTCTGTCTG
GACTATTGCTTTCCTGGCTTCTATTCTAGCCTTTAATACAGGCAG
GCCTGCAGAGCGGCCAAAATGCTTTTTGAAAAACAAAAAGCAAAT
GTGATTCTCCTACTCAAAACCCACCAATAGTCTCCCATGACTCTC
AGAA

In some embodiments, the intronic domain that promotes trans-splicing primarily in the liver comprises or consists of sequences from the gene CCNB1IP1. In some embodiments, the sequences from gene CCNB1IP1 comprise or consist of:

(SEQ ID NO: 731)
TTAAGTATCATTTGTTACTATTATTTTCTCTACTGAGCGTGGAAA
GCTGGCTGAAAATTCCAGAGTGTAATTTCTGTGGGTCTGTTTCTC
TAAAACTTGCCCCTCACGCAGCTTGTACAGAAGTTAGATTGGCGC
TTACTCCCAGCTGGTGGAAGATTCGGAATTAGCTGCGCCTCTGCT
GCGTGAGAGAGGGCTCAGCTTTAAAGCTTCCCAGCTCCTCTGCTT
TCCCGCTGCCCCCTACTCTGCCGGATGTAAAATAGGCCTCTTAAT
CTTAACTGTGGCTGCCAGGTGACATACCTGAGGAAACTCACTCAG
CGGGGCTATTGGGATAAAGGTGTGCGAGTGTTCTTTTCTATCGAC
CTGGAGGGCCAAGGCAACTTTTTTTTTTTTCCTTCTGGGACGGAG
TCCGTCGCCGGCTGGAGTGCAATGGCGCGATCCCGGCTCACTGCA
AGCTCCGCCTCCCGGGTTCAAACGAGTCTCCTGCCTCAGCCTCCT
GAGT

In some embodiments, the intronic domain that promotes trans-splicing primarily in the liver comprises or consists of sequences from the gene CCNB1IP1. In some embodiments, the sequences from gene CCNB1IP1 comprise or consist of:

(SEQ ID NO: 732)
AAAGACGGAGGGTCGCTCTTGTTGCCGTGGCTGGAGTGCAATGGT
GCGATCTCGGCTCACTGCAACCTTTGCCTCGTGGGTTCAAGTGAT
TCTCCTGCCTCAGCCTCCCGAGTAGCTGGGATTGCAGGCGTCCAC
CACCACGCCCAGCTAATTTTTGTATTTTAGTAGAGAGAGGGTTTC
ACCGTGTTGGCCACGCTGGTCGGGAACTCCTGACCTCAGGTGATT
TGCCCGCCTCAGCCTCCCAAAGTGTTGGGATTACAGGCGTGAGCC
ACCGCGCCCGGCCCACTATCGCTTAATTTAAAGAGCTGTAATGCA
TGTTTGTAATGCAGACTCAAGGAAAATTATAATTTCCTTAAATGT
GTATAGAGGATGTGTGAGAGTGGAAAGATGTCTATTCCAAGGTGG
ATAAAAAATAAAATGGTGGACTACCTCAACATGGAGGTGCCAGCT
GTCCAATTACAGCTGTCACTCTGTTTCTGGGAATTTCTCCCGATT
CTTA

In some embodiments, the intronic domain that promotes trans-splicing primarily in the liver comprises or consists of sequences from the gene PSMB5. In some embodiments, the sequences from gene PSMB5 comprise or consist of:

(SEQ ID NO: 733)
AGGACTACTTGCATGACCAAAGCCAAGATGGCAGGTACGTCCTTT
TAGAATTTAAGGTTAGTGAAGCCATCTATTTCCCCGACCCCCTTC
AGTGAAAATGGTCTTTCGCATCTGGCCCTTCTTTTTGGAGGCGTG
CTTGCCAGCAGTCAAAATGGCTCCATTCCGGAATAGATTTAATAG
GAAGTGAAGCTGTGACGGCGAGGCGTTGCCCGGCCTATCTTTGCT
AGGCGTTCTCAGAATTAGTTCTTTCTGCCCACACTAGACATGGCG
CTTGCCAGCGTGTTGGAGAGACCGCTACCGGTGAACCAGCGCGGG
TTTTTCGGACTTGGGGGTCGTGCAGATCTGCTGGATCTAGGTCCA
GGGAGTCTCAGTGATGGTCTGAGCCTGGCCGCGCCAGGCTGGGGT
GTCCCAGAAGAGCCAGGAATCGAAATGCTTCATGGAACAACCACC
CTGGCCTTCAAGGTGTGGAGCCAGCCCCCTTGCCAGGCTGAGTAC
TGAA

In some embodiments, the intronic domain that promotes trans-splicing primarily in the liver comprises or consists of sequences from the gene PSMB5. In some embodiments, the sequences from gene PSMB5 comprise or consist of:

(SEQ ID NO: 734)
TCTCAACCGTCCTCCCGTCTCTGAGATGGTAAATTACTACTGACA
CTTATTCAGGTCTGAACTGAGAATCTAGTTTGTGGATTAACTGGA
CCCTTTGCTTTTTCTTCTTCCATCTGCCTTTTAGGCAATTCATTA
CTTATTCGAATTTCCACCAGAAGGTAGGAGTCAAGAGAACGAATA
TTGGATATTAATAACCGGTTACTGTTAAAGAAGGGTTGCTGTATG
GCGACTGACAACAAGAGGTATAGCACTTTTATTTTGTTGCGTTGT
TTTACCACTAATTGCTGTGGTTTAACTAATATAGGGAAATGGCCT
CAGAATGGATCATTATTTTACCCAATTTTAGAAGGTGCCCTTAAA
CAAGATGGCAACCTCAGGCGCGTTAACAAGGTGCTGAAGCAGCAG
CGATGTAACCTGTTGCTTGGACACCAGGCTGGAGTGGGAGGACTC
ACCGCTAAGGGTTCAGAACCGATTTTCAACCAGTCACGCCCTTCT
CAAA

In some embodiments, the intronic domain that promotes trans-splicing primarily in the liver comprises or consists of sequences from the gene CTAGE5. In some embodiments, the sequences from gene CTAGE5 comprise or consist of:

(SEQ ID NO: 735)
AAGTCCGGACGTGGTTGGCAGGGCGCACCGGCGCGTGCCCCGCAG
GCCATTTTTCTGACTGGAATGACGGCGGCGGCTGGCTTCTCGGGG
CTGCCGGGGTCTCCCGGGGTACGCCGCCGTCAGGACTTCTGCAGG
GCGCAGACAGCAGGCTCCAGCGAGGCTGCGGAAGGAAAGCCTTGG
GTGTCGGGGCTGGGCCTGGCGTCGGCCCCTGGGTCGGGGGGGTGG
GGGTTGCGCCGAGACGCCTCTAGGAGAAGTCTCGCCGCCGCCGCC
ACCGCGGCCGCCCGAGGCGGGTGCCCTTGGGACATAGCCCTGGTC
TTTGGGGTTGTGCGGCTCACACGACAGCGCGGAGTATGAGGCCGA
ATCTCATCCTCTAGTCCCAAGCCTCTCCACTACCAGGGCTGGGTG
GCTTCGCCAGGTAGAGCGCCGGCCCCTTTAAGAGCAAACGACCCG
GACACGTCTGCGAAGCTTGCGCGAAGAAGGGGAAGTTTGCGGCTG
TCCG

In some embodiments, the intronic domain that promotes trans-splicing primarily in the liver comprises or consists of sequences from the gene CTAGE5. In some embodiments, the sequences from gene CTAGE5 comprise or consist of:

(SEQ ID NO: 736)
TGAGCCCAGGCGCGATGAGTGTTACGTGGCCCAGGGGTCGCGGGA
GCCGCCGGGGGAAGGAAGCAGTAGACCGGCTTTGGGGTCTAAGCC
GCCGTGGTCAGAGGTCCCGAAGCCAGTCCTCGTCTGCTGCCCGGC
TCCCGCCCGTGTTCGAGGCAGTAGTTAGGCTCGTAGGGCGCTTGA
GTGGTTTTTGTGTGATGGAGGAGGACCTGGGGCTCTGGGAAGGTG
AAGAAAGCACTTGCTTAGCTGGACTTTGCAAAGATGTAAATGTTT
CCCGATTCCTAAAACTAATTAGCCTTTCCGCTATGTTGTTTATTT
TGAGCAGAGACACGTGGTTCCTTCGACTGTGAGAAACTTGAGGAC
TGTTCCTTTAAATTAGTTACATTTACTGATGTAACTTCTCGTTGA
ACCTTGAAAGAACACCTGAGGTTGGGCGGGGAGTTGAAATTTTGA
TTCTGCATTTTTTTTTTTTTTGGTAAAGAATGAATGCCCTTTTGA
GATG

In some embodiments, the intronic domain that promotes trans-splicing primarily in the liver comprises or consists of sequences from the gene SERPINA10. In some embodiments, the sequences from gene SERPINA10 comprise or consist of:

(SEQ ID NO: 737)
GTACTTTGATAATGTTCTGCTCTCCCAAGGCCACAGGGCCCTACG
ATTGTCTCTCCCTTTCCTTTCGTTAGGCCAGCATATGATTAACGC
TACGTGATTTTCTATGAATGTGTTTTCACGTTTCAAAAACAGATT
GATACACATATTGAACAGTGCCAGACGCTGTCATTTGAGGCCCTT
CCCTGGTATCCTATGTGCTTGTAGTCCTTATTATTTTCAGAGCAC
TCTACATAGCTCCCCTCTGACACTTAGAAGCATAGGGTCTTTCCA
AAAAACAGGGGGCTGGGGGATTATCTGGGGGATTTAGGATTGCAT
CATTGCTCCTTCATTTTTACTTTTTGACCAACTCTCTGCCCTTAG
ATTCCTATTATAGAAAATAGGGACACTCCACCTACTACAGTGTTA
GAGGCTAAATGAGACAATGAATGTAAAGTGCCCAGATGGGCTTGG
CACATAGCAGACACTGAGTATCTATTGTTTACTTGTTCTTCCAAA
CTGC

In some embodiments, the intronic domain that promotes trans-splicing primarily in the liver comprises or consists of sequences from the gene SERPINA10. In some embodiments, the sequences from gene SERPINA10 comprise or consist of:

(SEQ ID NO: 738)
TCCCCCTTCCTTGCCTAACGGGCATGCAGGCACTCCAATCCCCAG
CCAAACAGGGCACTGGGCAAGGCCGGCCACCCATCTGGATGGGCA
GCCTGACGACCAGATGGTCAGGGCAGTGAATGAAGCAGATCAAGG
AAAGGTGTGTGAGGACCCCTGATTCCACCTGCTTGGACCCCCACC
TTCTGTGCTGCCTCCTGCTCCCAGAGTGGACTCTCTTGCCCTGGC
CCTCAGGGAGGAGACGGGATGAATGAAAACGGGGTCAGGACTGAG
AGCTGCCTGCCGGCCTGGCAGGGAATGGGAACTGGAGGAGGTTTT
GCTCTGTGAAATAATGTCCCCTCTTTGGGTGAGCAAATGTCACCC
ACACTTGCTCTAGGTCTCCCTGGGGCAGGGCTAACCTACTTGAGC
CACAGGAAGGAGGCAGGGTCCCTGAAGAAGCTTTTACTATCCACA
AAGACATTTTAGGAGGCATTAAAACCATCTCTATCCTCTCCTCTC
CACA

In some embodiments, the intronic domain that promotes trans-splicing primarily in the liver comprises or consists of sequences from the gene SERPINA5. In some embodiments, the sequences from gene SERPINA5 comprise or consist of:

(SEQ ID NO: 739)
TCCCCTCATTCTTCTGAGCCCCAATTCAGTGATTGGTGCAATCAC
AGGCTTGGCTACAGTGACCCATTCATTGCAGGCATGGTGAGTCTC
TCAATCCCTCTCATTTCCACTAGAATCTAACTGTTGGGATCTATG
ACCCAGTCAGCATAGCAGGCCTGTGGGGAGCTCTCAGGTTCAAGC
ATATGCCCCCCCTAATCTACAAGAAATTAGCTGCAGAAAACCAAG
GAATAGAACCTGGAAAAAGAGAGGGTTTGCTAGAGCTGTCCCTTT
CCCTGTCTCTGGAATGCCAACAATAGGGAGGCTCTTTGGTCTTGT
CTCTCAGGAGTGCCCATGCCATTCCAGGAAAATGATGGCCCAGCT
GGTGGTGTAAGGCTTGGGGGGCAGCGAGTGGGCATCGTGGTGAAA
GCCTCGGGATCAGGGAGCTGCGTCTGCAGGCAGGCCTGCTGGCCG
GAAACCTGCCAGGAAAGGAAGGGGCTGTCCCGGGGCGGGGCCAGG
GAGG

In some embodiments, the intronic domain that promotes trans-splicing primarily in the liver comprises or consists of sequences from the gene SERPINA5. In some embodiments, the sequences from gene SERPINA5 comprise or consist of:

(SEQ ID NO: 740)
TAAGTACCGATGCCGCAAAGGGAGGTCCCCAGGGCTTGAGGGCAT
GTGAGGCGAGGAGAGGATGGACTCTAGAGTTTTGGGGTTTGGGGT
CTGCAAAGCTCTGAAGGAGTCTCATCTCTGCAGTTTCAGGTATCC
AAGGCAGCAGAGGTGAGTGGGTCCCCCGAGCTCTGTGACCTTATG
CTCCACACTAACTCTGGCAGAGCCTCCGTTTCCTCATAGGTAAGA
TGGAAATAATTACACCCTCTGGATGGTGTGACTGAAGATTAAATA
CAGCGGGTGCTCTCACTCAGCACATCTGGCCATGTCTGCAGACAC
ATTTGGTTGCCACAACTGGAAGGGGGGTGGGGGTTAGTGACATCT
AGAGGCCAGCGATGCTGCTGATGATCCCACAATGCCCAGGACAAG
ATCACAAAGCATCATCCTGTTCAAAAGGTCAACAGGATCAAGGTT
GAGAGACCCTGAAATAAGGCCATGGGGACAAAATGTCGGCTGGAT
AGGA

In some embodiments, the intronic domain that promotes trans-splicing primarily in the liver comprises or consists of sequences from the gene PPP2R5C. In some embodiments, the sequences from gene PPP2R5C comprise or consist of:

(SEQ ID NO: 741)
AGCGTGGTGGCGGGTGCCTGTAATCCCAGCTACTAGGGAGGTTGA
GGCAGGAGAATCACTTAAACCTGGAGACGGAGGTTGCAGTGAGCC
GAGATCACGCCACTGCACACCAGCCTGGGCAGCAGAGTGAGACTC
TGTCTCAAAAAAAAAAAAAAAAGTGAGCAATATCTGTGGTTAATC
TCAAATAGGGTGGCTTTCCGTGTCATTTCCCTTTAGTTGTTTTTA
AATGCAGATGTTGCTTTAATTAAATAATATTTGAAATAAGTGGGG
TCCCAACTATAACCTTATATCTTATTGTTTTTAAATGTTGATATT
TTGGTGTTTTCAGTAGAATTTGAGCTGTTAACAGTGATTAGGTTT
TTTCAGTTTGTCATCGATGTATGTGACCTCGTTATAATACACATG
GATTTTTGTCTGCTGGTTGTGTTACTCATGAGCAATGTGTTCTAA
TTATGGTATTTTAACTTATAACTTATGAAAAAGTTATGTTTCACT
CTAG

In some embodiments, the intronic domain that promotes trans-splicing primarily in the liver comprises or consists of sequences from the gene PPP2R5C. In some embodiments, the sequences from gene PPP2R5C comprise or consist of:

(SEQ ID NO: 742)
TGGGTGGTGAGGGTGTCTGTATATGTGTGTTTACATTTGTAGACA
GTTTCTGTACATGTATAATTAGGAATGGGTTGTCGGTTTTGGTTT
TTTTCCCAGCTTTATGTAATTGGTCAGACTTCTCAAAGGGGGGTA
GCACTTGATAATCGCTCTCTTGGAACTCTGAAGCCTTTGCATGGA
GCAGTGTTCACCCTGGGCAGCCTGTGGCCCCTGGGGTCACAGAGC
TGCTTTCCAGGGGGTTTGCAAGGCCGTCACTGCCTCATAAGATTA
CCAAGATCTAATTTACCTCTCACTGTGTGGACGTTTGCCCTGGTG
GTGCAAAGGCAATGGTGAGGAATATGGCGCCTTTCTTTCATCGAG
CAGGACAGCGGTGTTCAGCTAGCAACACACCAGTCTGACCCAAGA
ATGTCCTCGATAAAGCATGAAGTTACTGGTTTTTTAAGTCTCAAC
TCTTTTGTATACCTTTTTAATTTTGTTTTTGTTTGGGTTTGTTTT
TAGT

In some embodiments, the intronic domain that promotes trans-splicing primarily in the liver comprises or consists of sequences from the gene SERF2. In some embodiments, the sequences from gene SERF2 comprise or consist of:

(SEQ ID NO: 743)
AGGATCCGGCGTCCGGAGCCCGGGATCACCTGCCTAGCGGGCTTC
ACTTGGAGAGGTGGGGAGGCCAGAGCTTGCGGAAGCCGCTCCCTC
TCCCCTGCACCTTCTCCAGGGTCGCCGGCTGAGGCCCAAGGTCCC
TAGGGGCAGTGACTAGAGAAGAAGGGGGCCGGCCGTACCTGTTGC
CGCTGACACTCTTGCAGCGCCCGCAGGGCAGCGTCGTTGAGCCTG
AGCAGTAAGAGGCTAGTCCGGGCAGCTTGCGTGAAGCAGAGCCGG
AGCTGTCCTCTCAGAGGCTCCTGGAGCTCCTCCATGGCGACGAGT
TCGAGTGCAAGCAGCACGGGGGCCACAGGCGAGGGCCACCACCGC
CACCTCCTCTGTTCAGGGTTTGGTTGGCACACCCGGTAACCCACG
TCTGCCCCACCCTCCGCCAGGAACCCGCCCCCGGCCCCACCTCCC
GCCAGCGCCACCCCAAGCTTTCCCAGCCGCGTGGGCGGCGCTTCG
CTCT

In some embodiments, the intronic domain that promotes trans-splicing primarily in the liver comprises or consists of sequences from the gene SERF2. In some embodiments, the sequences from gene SERF2 comprise or consist of:

(SEQ ID NO: 744)
TAATTGGGGCCCTGCGGTTGTGGTGGCAGCAGTGAAAGTTGGCTG
TCACCGACTGGAGCGCCCCCAGTTGGTGGGCAGGGATACGGGCGC
GGGTAGTGGCAGGAAAGTTGCAGGGCAGAGAGCCAGAACCCAGTC
CTAGGTCTCACCTCGTGGCCTAAACACGCGTCAGAGTGGTGAGAC
CGCTCCCTGAGGAGGGCCAGGAGCTGCCATGAAGTCGCCAGGCAT
TCCAGAGGGCCAGATCCTGGTGCACTGAGACCCCAGCACCAACCT
CTGGCCAGCCCTTGGTGTCTCCACTTGTTCCTGTTTGATTATGTT
TCCCAAGAGCCCCGATGTTGTCCTGTTTCCTAAACAGGATCCCTC
TGTTAAATCCCTGGACCTGCACAATCCCCGGGAACCTTAACGTCC
TCTACTACAAATAAAATTACCTATACATTTGTCAAAACTCATAAA
AAAAAAATACATTTTTTTTTTTTTTTTTGAGACGAAGTCTTGCTC
TGTC

In some embodiments, the intronic domain that promotes trans-splicing primarily in the liver comprises or consists of sequences from the gene AQP9. In some embodiments, the sequences from gene AQP9 comprise or consist of:

(SEQ ID NO: 745)
GCCCGCTGCTGTGGCAAGAATGGCCTGGGGTCAGTGCTAGCTGTG
GCAGTGGCTGCAATACCCCTGGCTCTAAGGGCATCTTCAGTATAC
TGCAACACCCTCAAATTCTGGAGTGGTATTAGGGGTTACTGAGAG
CCCTCTGGGAATTATAAATTATTCCTAGAGCAAGACCAAGCCCCA
CTTTGGCTTACAGTTGACCTCGAAAACTCCAAGAATTCCCCCAAA
AGAGAACTCATTCCCAGGACCCTATCGAAGAATAATTTGTCCAAG
TTTGCAGTGGGCCATTCAGAGAGGCCAAAGGCCTTCTTCCTTTGA
CCCTCCATCCTGGGATTCACCTGGGCATCTCTAAAACTATCATTC
CTCGGAAGTAGAGAGAGACAAATGACATGGCTATGCCAAGCTCAA
TCTGAGTGACTGGTTGTTTAGCACAAGGCTTGGCACAGGCCTCCT
TCAACTCTTCCCAGGGAACACTAATGTGCCAGAGCTTTCTCTTTC
ATAG

In some embodiments, the intronic domain that promotes trans-splicing primarily in the liver comprises or consists of sequences from the gene AQP9. In some embodiments, the sequences from gene AQP9 comprise or consist of:

(SEQ ID NO: 746)
TAAGTGTAGATTCAACAAAGACTTAACTTTGGTGAAAAGATATGC
TCTCATAAGGGGAATGCATTGGAGAATTAGAGACCAATCAGAAAG
GAGAGATTATATTTCCAAAGGCAGAGGTTGCTGGGCATAACCCAA
GCTTTCTTGATAATCGTTTTACCTTTACAAGGAGCTGATACCACC
AGAAAAGTTTTCTCCTTCCTCTTTAATACGTACACTTGTTTAACA
TTAGTTGAGATCCAGAAAAGAGTGAATGACTAAACATTTCAATCC
CTGTAGACACAAATGGACTTCACTTCACTATTGACTCTTTTGAAC
AAGTGGTCTAAGTGTGGAGGCCACAAGAGAAGATATTTCTATTTC
AAATCAAAGACTGTGAAGGGTGGTAGTTGAGCCTAGGGGATCATC
AATCCTCTCTGTCTCACCATGTGCATAGGTTTATTTATTTAAACT
CTCAAGGTGGCTAGAAGTATCTCTGCTTATTGACCTTTAAACAAG
CTTC

In some embodiments, the intronic domain that promotes trans-splicing primarily in the liver comprises or consists of sequences from the gene ANPEP. In some embodiments, the sequences from gene ANPEP comprise or consist of:

(SEQ ID NO: 747)
GTAATCCAGCCCTGCCTTATGGGCAGCAGCCCCCACCCCATACTC
CTAGCTCCTGGCCCATGTTCTGAGGGCTGTGATGGGCCATTCCAT
TCAACAAATGGACTTGACTTCTTTGGTTCCAATCTCCACTTTGCT
GCTGCTTCGCTGTGTGACTCTGGGCAAGTTACCATCCCGCTCTGG
TCTCTAGTTCCTCTTCTATAAAATGAGGCTAAGAGCAATACTCCA
TTAGTTATGTGAGGATTAAAGGAGAGAATCCTAGGCCAGGCATGG
TGGCTCATGCCTGTAATCCCAGCACTTTGGGAGGCCGAGGCGACC
TGGGGTCAGGAGTTCGAGACCAGCCTGACAAACATGGAGAAACCC
CATTTCTACTAAAAATACAAAATTAGCCAGGTGTGGTGGTGCATG
CCTGTAATCCCAGCTACTCTGGAGGCTGGGGCAGGAGAATTGCTT
GAACCTGGGAGGCAGAGGTTGCAGTGAGCCAAGATCGCACCATTG
CACT

In some embodiments, the intronic domain that promotes trans-splicing primarily in the liver comprises or consists of sequences from the gene ANPEP. In some embodiments, the sequences from gene ANPEP comprise or consist of:

(SEQ ID NO: 748)
CGAGAATCTGCAGGAGATGCCCTGGATGGCAGGGACTGGAGGGTC
CCACTGATGGCCACGCCCTCTCGCTCCACCCACAGGCTGTGAACA
ACCGGTCCATCCAACTCCCCACCACCGTGCGGGACATCATGAACC
GCTGGACCCTGCAGATGGGCTTCCCGGTCATCACGGTGGATACCA
GCACGGGGACCCTTTCCCAGGAGCACTTCCTCCTTGACCCCGATT
CCAATGTTACCCGCCCCTCAGAATTCAAGTGAGCAGATGGGGTCA
CTGAGGCATGGTCAGGTGGGTGGGGTAGAGGGCAGTTACTAAGGG
ACCATAGTAGGACTGGCCTCCTGTGGTCCCAGCCCCAGCCACTGT
AGACAGACCCTCCCTCCAGAGCCAGGAGTTCAGGGGCACTCGGTG
CCAGAGCTGGGCAGAAGCTGGGAGCCTGGGCAGCTGCTCCTCACT
GGCAGTGACCACACCGCCATCTCTCACCACCTCTGGATCTTTAAT
TGCA

In some embodiments, the intronic domain that promotes trans-splicing primarily in the liver comprises or consists of sequences from the gene PCSK6. In some embodiments, the sequences from gene PCSK6 comprise or consist of:

(SEQ ID NO: 749)
ATGAAAATGAAAATGACAGATCTGAGGAAGAGGGAGAGCTCTCTT
GAAACCCCTTTCCCAGATTTGCCCTCAGTTTTAGGAATGAGGTAC
GCTTGGGCATTCCTGTTGCCTGCATGGTTCGGCATTCTGGCAGCC
AGGTTCGCCCCAGAATAGATAAGTGTGTTTGATTTTCGAACATTT
GACTTTATTACCTATTTCCAGAAACACTTCGTGTTCAAACTACGT
CTCTACAGGCATTTCTACTTGGTCTTTTTCTCCCCTGACATTTCA
CAGAGTCCTAACTGTGCTGCATGTATAACTTGTTCTTTCTTTTTG
TTACTTGCATAGCAAGCATGTTTCTATGTCATTAAATATCCTTTG
TAAGCAGCATTTTATAGATTTTTATAATACTTCACCAACAGAAGC
ACCATAATTTATTTAAGCATCTCTCTATTGTTGGACATGTAGATT
GCTTTCCATTTTTTCCTATTATGAGCGGACAGCTTAGTACAAAAA
GCCT

In some embodiments, the intronic domain that promotes trans-splicing primarily in the liver comprises or consists of sequences from the gene PCSK6. In some embodiments, the sequences from gene PCSK6 comprise or consist of:

(SEQ ID NO: 750)
CACTTCCTTGCCTCGGCTCCCCCAGCCTCCCCCACGGTGAAGCCT
GGGCGTTGGAATGGCTGCTGACGTTCCCACCGGGGAGGAGCAGCG
GAGGCGCAGGGGCCGGTCCGACCCAGCGGGACCCCGGGGGGGCGG
GGGTTGTGGCTTTTCCCTCCACTTCCAGGTGAGGCTTGGTATCAG
GCCTCCCCAGCATCTGGCTGCCTGGAGGGGGCAGGTAACACAACC
CAGAAGGAAGGTTTACCCAAGGAAAGACAGGACATAAAGTGTATG
CCCTTCCTGTCTGAGGGCACATTGGTCTCACAAGGCCTGTTTGGA
AGACTTCCCCCAGCTAAGCAACCACCCCGTTTCTTTGTGGAGCTG
CAGCCAGCTTGAAACACAGCTCCTTCCATTTGTCAACCCCGGGCG
CTTCCTCCTGCTGCCCTCCACTCCTGTTGCCCTGGGATTGCACCT
CCCAATAAAGCTTCAGCCCATGGGCTTCACTTCAGTCCCTTGTTT
TCCA

In some embodiments, the intronic domain that promotes trans-splicing primarily in the liver comprises or consists of sequences from the gene ACSM5. In some embodiments, the sequences from gene ACSM5 comprise or consist of:

(SEQ ID NO: 751)
GTTAACTATGGAAAGAAAAGAAGAAAGACAGGCAAGAAAGAGGGA
AGAAAATAGGAAGAAAATGAAGAAGGAAGGGAGGGAGGAAATAAA
GGAAAGAGAAAGGAAAAGAAGGAGGAAAGGAAGGGAAAAAGATGA
ATGGAAGGAAGGAAATAGGGAACAAAGAAGAAGGGAGGAGAGAGG
AAAATGGAGAATGAAATAACGGGGAAAAGAATGAAAAAAAGGAGG
AAAGAAAATGAAGGAGGGAAGGAAGAAAACAGGAAAGGGGAAAGA
AAGGAAAGAAAGAGGAAAGGAAGGAGTGAGCAAGGAAGAAAAAAG
AAAAGAAAGAAGGGAGGAAGAATGGAGAGAAATGGAGAGAGGAGA
AGGGAAAGAAAGAAGAAAGTGAGGGAGGAGAAAAAAGAGGATGAG
AGAAAGAGTTATCAGGCAGGGTGAGAGAAAGGCATGGCCCAGGAA
AGAAGCTAAGTGGAAAGGCTCTTCTTTATCTGTACTGCGTTCTCC
CCAG

In some embodiments, the intronic domain that promotes trans-splicing primarily in the liver comprises or consists of sequences from the gene ACSM5. In some embodiments, the sequences from gene ACSM5 comprise or consist of:

(SEQ ID NO: 752)
TAGCAGCCTCCCCCAACTTCTGGCAAGGCCTGGCATGGAGAGGAG
AAAGACACACAGGCCTGGTTGGCACGGGCTGCTGGGCTGCTGGGA
GAGGCCCAGGGTGTAGACACTCACGTATGCACCTCTGTGATGATT
TCCTTACCAGATTGTGGATGATGAGGGCAACGTCCTGCCTCCTGG
AGAAGAGGGGAATGTTGCCGTCCGTATCAGACCCACTCGGCCCTT
CTGTTTCTTCAATTGCTATTTGGTAAGAGACGGGGAACAGCCGTT
CTCATGACAGTGACTGTGTGCTAAGCATTGTGCACCACACTTTGT
AAATATCTATTTGTTTAATCCTTACAATGACTGCATGAGGTAGGT
TGCTACTATTATTAATAAAGTTCTGTTATTGATAAGGAAGACTAT
TATTGATAAGGAAGCTACTACAAAGGGGAAGTGACTTATCCAGCA
TCATGCAAATAGTACAGGCAGGACATGGGATTTAAATGTGGGTGT
GTCT

In some embodiments, the intronic domain that promotes trans-splicing primarily in the liver comprises or consists of sequences from the gene ACSM3. In some embodiments, the sequences from gene ACSM3 comprise or consist of:

(SEQ ID NO: 753)
GCTGTGGATCAGAGGACCCTGTGCCAGATCATACAACTCCTTGCA
GTTCATGTAAGGACTCGGATTTTACCTGGAGTGGAAAAAGAAGCA
CTGAAAGATTTGAGCAGGGGAGTAACCTGATAGCGTTTATGTTTA
GTCCTGCCACTTCGACAGATAAACGCACCAATGGGCTTGATGAGA
TTTAGGCCAACCCATAACCGCCCCTCAACTTCTTTCCTTTCAATT
TCAAAACTCCTCTATGGCTTCCTCCATCTGTTCTTCCTTCTGAGA
AGTGCTCTCTCTGCCCCTTTACAGAACTAACCACTTCGGCAACTC
CTTGGACACTTTCCTTCTTGTTAATAATTTGCTTTCTCCGCCCCT
CAAAAGCTTGCTGTTTCTGTAAATCATTACCTGTAAGAGGAACCG
CTGGGAGTCCTGTAAACTTTAGCCCAGAGCTTGGCTCCTCCTCCA
GAATGTCTCCACCAATCAAGGAAAGTGTTTTGGGCCAGTCTTGCT
CCTC

In some embodiments, the intronic domain that promotes trans-splicing primarily in the liver comprises or consists of sequences from the gene ACSM3. In some embodiments, the sequences from gene ACSM3 comprise or consist of:

(SEQ ID NO: 754)
TGAGTACCTCATCTCCCCTCCTGTGTAAGATTTCCTGAACTGGCA
CATCTGTTTTTTGAGCAAAGATAACAAACAGATGAACAAAACCAA
CAATCAAAAATGCTGTCATTAAAGTCTTGGGCAGCCAAAGTTTCT
CTCAGAATTTCTCAGTTGTGTGATACTATCTATTAAGTGATGAGG
AGTATGCACACACAAAAGGCTATAAATGTAGCAGCTGAGTTTTCA
TGTTGAGCCTTTTGGTGCTATTTGATTTTTTGAAAAACTATGTAC
ATGTATTAAGTTGATAAATTTTTTTTTTAATTTTAATTGAACCAG
ATGCGGTGGCTCAAGCCTGTAATCCCACCACTTTAGGAGGCTATG
GTGGGCAGATGCAGATCACTTGAGGCCAGGAGTTCGAGACCAGCT
TGGCCAACATGGTGAAACCCTATCTCTACTGAAAATAAAAAATTA
GCCAGGTGTGGTGGCAGGTGCCTGTAACCCCAGCTACTTGGGAGC
CTGA

In some embodiments, the intronic domain that promotes trans-splicing primarily in the liver comprises or consists of sequences from the gene TMED6. In some embodiments, the sequences from gene TMED6 comprise or consist of:

(SEQ ID NO: 755)
GTCTGTTTTCATTACCATGGCTATTATAATAATCATAGGGTCCTA
TGAAATTACAGAAATAATGAGGAAGTATACAGAGATGTTTTAACA
GTAGGAAAAAAATTCAGCCTTTAACACCAAAAACCAGTGTTCCTG
GAGGCTGCTGACCTCCAAGGATGCCAAGTGGCAGTCTACTGACGA
TTTGGGGGCAACACTCCAGGTGAGCATTCTAAGTCTAGGGTTCAG
CCGCTCAATTTTATCTCATCATCTCCAGTTAATCAGATTCTGGGA
AAGTGACCAAAAAACAGAGACCCATTTATAATACAGAATTCCAAT
TAACAAATGGATTACAGGGAAGCATGTCTTTCCAGTCATCAGAAT
ATTTGAGTCAGTTACTAAAAGCAGATTCAGAACAAAAGAAAATGC
ATGATATCCATGTGAGATATCCACACTCCACAGCAAAGCACTCCT
CCAGCCAGGTCAAGCCTGTCCCACTTTGGAGAGCAGCAGTGTCTT
AACT

In some embodiments, the intronic domain that promotes trans-splicing primarily in the liver comprises or consists of sequences from the gene TMED6. In some embodiments, the sequences from gene TMED6 comprise or consist of:

(SEQ ID NO: 756)
TATAGGCGCACGCCACCATGCCAGGCTAATTTTTGTATTTTTTAA
GTAGAGACAGGGTTTCACCATGTTAGCCAGGCTGGTCTCGAACTC
CTGACCTTGTAATCTGCCCACCTCGGCCTCCCAAAGTGCTGGGAT
TACAGGCGTGAGCCACCGCGCCCGGCCCAGAATCATACATTTTAG
TGGATCCACACTAGCTGTGTGTTCCGAGCAAGGCATGTCTTCTTC
ACACGGGTGCTGTTGGCATTCTGGGCAGTTCTTTGTCCTGCTGTA
CTGTTTCTTACATTGCAGACGTTCAGCAATGCTGTGCTGCCTCCC
CTTAACACCATCACTGTGATACCTCAGAATCCTCCAATCAACTCC
AAACCCCAGCAGGGGGTTGGGCAGTCCCACCCCAGACAGAGACCG
CTGTGCTGATGACCGTGGGACGTCAATCTTACCACTCAGCTGGCT
TCCTCGTGATGTAGCAGGGGTTCTGCTACCTATTTAATGTGTCTT
GGCA

In some embodiments, the intronic domain that promotes trans-splicing primarily in the liver comprises or consists of sequences from the gene HPR. In some embodiments, the sequences from gene HPR comprise or consist of:

(SEQ ID NO: 757)
AGGGGGCTGGGCGTGATGGCTCACACCTGTAATCTCAACACTTTG
GGAGGCTAAGGCGGGTGGATCACTTGAGGGTGGGAGTTTGAGATC
AGCCAGAGCAACATGGAGAAACCTCGTCTCTACTAAAAATACAAA
AAATTAGCCAGGCATGGTGATATATGCCTGTAATCCCAGCTACTC
GGGAGGCTGAGGCAGGAGAATTGCTTGAACCTCGGAGGTAGAAGT
TGTGGTGAGCTGAGATCATGCCATTGTACTCCAGCCTGGGCAACA
GGAGTGAAACTCTGTCTCAAAAAAAACAAAAAACAAAAAAGAACA
CCACGGGAAAATCAAGTGTGAAGCAAGAGCTCAACTCTTAACAGG
GGCAATGTTTGGAGTTTTGTTACTGGAAAAGATAGAGACCTTACC
AGGTCCAAAGTTTGTAGACACAGGAATTACGAAATGGAGAAGGGG
GAGAAGTGAGCTAGTGGCAGCATAAAAAGACCAGCAGATGCCCCA
CAGC

In some embodiments, the intronic domain that promotes trans-splicing primarily in the liver comprises or consists of sequences from the gene HPR. In some embodiments, the sequences from gene HPR comprise or consist of:

(SEQ ID NO: 758)
TGGGTCCACAGCTTTCCCTCCTGCCTTTCCTCTGGTTCTTTATTT
CAGTCTTTTTTGCATACATTGGTACAGATGCAGAAATAGAACAAA
GAAACAGGGCAAATGGGCTAAATTATAGTGAACCAAAGGGCTTAG
TGTGTTAAATCTTCTCCTTTTCTGCATCCATAGAAGACAGTGCTG
CTGTCTTTCCCAGGAGATAAGATTTACTCTCAGGAGTGTCTTTTT
CCTTGAGGTTACGTTTTTGTCTTTGTAGGGTATGTCATCAGCTCC
CGTGGTAGGCTTCCTGGCATTCGGAATATATTTACTAGCAGATAT
TTTCCTCTTTAAAAATGTATAATAAGAAAGACTAATAGTAACACA
TTTGAATGACACAATTAATTGACTAGTACTTGGGATACACACTAG
TACCTGGGATACATCTAATTAAGACACTTAGATCTTAAAAAATAA
AAAGACTTTTTGAAATGTTGAAATAATAAGACTAGAAACTTTTTT
TTTT

In some embodiments, the intronic domain that promotes trans-splicing primarily in the liver comprises or consists of sequences from the gene SERPINF2. In some embodiments, the sequences from gene SERPINF2 comprise or consist of:

(SEQ ID NO: 759)
TGTCCCCCAGGCTGGAGTGCAGTGGCTTGATCTTGGCTCATGGCA
ACCTCTGCCTCCCGGGTTCAAGCAATTCTCCTGCCTCAGCCTCCC
GAGTTGCTGCGATTACAAGCGCCCGTCACCACAGAAGCCCAGCTC
ATTTTTGTATTTTTAGTAGAGATGAGGTTTTGCCATGTTGGTCAG
GCTGGTCTTGAACTCCTGACCTCAGGTGATCTGCCTGCCTCGGCC
TCCCAAAGTGCTAGGATTACAGGCGTGAGCCCCCATGCCCGGCCA
CACTGCTAGAAACTTTAAAAGCATCTCATTCGGTTACGTGTCCGC
CTGCAGTGGGACAGGTAGTATGACTCCACCCCACTGTGAGGAAGT
GGAGGAGCAGTGGGGTCAGTCACACGCCTGGCAGGATGGCCAGGA
GCCCCATTGTCTGCCTTAGGAGCACCTGCTGGCCCCACCCCCACT
TAGCTTCGGGGCTTTCTGTCCTCATGCTCTTCCCTTCCCTTTTCT
GTAG

In some embodiments, the intronic domain that promotes trans-splicing primarily in the liver comprises or consists of sequences from the gene SERPINF2. In some embodiments, the sequences from gene SERPINF2 comprise or consist of:

(SEQ ID NO: 760)
TAAGGAGGAGGGTGCGGGCGAGCCCCCGAGGTCAGGCTGGGCAGG
GCGGGTAAGGAGGAAGCGTCTGGCTGGCAGTGGAGCTGAGTCAGA
GCTGTCCTGTGTCCTGCTCTTTTCCAGGGCTTCGGGAGCGGGGAG
AGGGTTGAATATGAGCCCCCAGACCCTCTGTCCTAGAGGAAGTTG
GGGTGGCCCGGGGAATGGGGCTCTGCTTTGCTGCCCAAGACCTCG
CATTGCAAGGAGAGTCTGGGTAGAGAGCCTGGGATGGCATGTGAG
CCTCGCTGGCTTCCCTGGAAGGGGAAGGAGGAGAAGCTGGGGTGT
CACACTGTTAAAGAAATGCTGGGTGGAGTGGCTCATGCCTGTAAT
CCCAGCACGTTGGGAAGCTGAAGTGGGTGGATTGCTTGAGCCCGG
GAGTCCGACACCAGCCTGTGCAACACATTGAAACTCCGTCCCTAC
AAAAAATACAAAAATTAGCCAGGTGTGGTGCATGCTACTGGGGAG
GTTG

In some embodiments, the intronic domain that promotes trans-splicing primarily in the liver comprises or consists of sequences from the gene SLC13A5. In some embodiments, the sequences from gene SLC13A5 comprise or consist of:

(SEQ ID NO: 761)
CTGTGCTGCTGACTTGTCTCTGGTTTTCTGCAGTGTGGGGAGCCC
AGGGAGGTGGATGAATGAACAGTTAGTTACGCCCTGCCCACCTGC
TGGGTGCCAGGCCTTCCTGTCCCTGTTGAATCCACTAGTTATCTC
GTTCGATCTTTGCAGCAACCCTGTGAGATAGGAAGGTGTTATTAT
CTTGCTTTGTCTTTCAAAAAAAAAGCGAGGCTCAGGGAGGCCAAG
GGAAGTGTCCAAAGTCACACATCAAGTTACTGGCAGTTACAGTTC
CAACCAAGAGCTTCCAACTCCATACCCCCTGCTCCTTCTGCTAGC
CATGAAGGGCTTTGGCCTTATAGGGCTTGTAGGGAAAGGTGAGTG
GCCAAGAGCAAGTCCATGCCAAGGGAAGATCTCCAAACATGAGTC
CCTGTCTGTTGCCTCCCCTGAGATAGGCACAGGACAAGTGATCAA
TGAGACAGGGTGGTCCTTGCCCTAAGAAGCAAAGTGTTTGGTTGG
GGAG

In some embodiments, the intronic domain that promotes trans-splicing primarily in the liver comprises or consists of sequences from the gene SLC13A5. In some embodiments, the sequences from gene SLC13A5 comprise or consist of:

(SEQ ID NO: 762)
CCATGGACTGCAAACTTGACTCCAATCCCCAGTAAAATATCTACA
ACAGGGTAGTGAAGCGATGGTTAGTGACCATGAGGGAAGCTTGCA
GAGCAGGCATCAGAAAGAGCCTGAGGAGGTCCACAGGGAAGCTGG
CACGTCCTTGTAGGATAGTTAAGGCACTGGGGTGAGCAATGAACC
TGGACTCACGGAACACTGGGCTCTGTGACCGTTTCCCTGAATGGC
CTAAGCTGTTGCCTCCTGTCACTTCTCTGAGGTCATTTTCCAAAT
GCGCACGGGCATAGAGAACCCATCCACTCTGCCTACTTCCCAGGG
ATGCCTTGAGCACTGAGGATACCTGGGGGACATGAAGTCGCACTG
TCCTGGGGGTCGGGACACCCCAGCCAGGGACAGAGCATGGCACAG
GGACATCGAGGCCCAGTGAGCCGACCCTTTGTCCTCCTCTCTGAG
AGCACTAGTCCCCAGCAGGCCTCAGGGTGCTGACTCTGTCTCTTT
TCCA

In some embodiments, the intronic domain that promotes trans-splicing primarily in the liver comprises or consists of sequences from the gene ASGR2. In some embodiments, the sequences from gene ASGR2 comprise or consist of:

(SEQ ID NO: 763)
GTGAGGCCACGCTTCCCACAGGCAGGAGGAAGCCCTTGGAAGGCC
TGCTCTGGGAGGCTGGATCAGGCTTATTTTATCCATTTTTTTTTT
TTTTTTGAGATGGAGTCTCGCTCTGTCGCCCAAGCTGGAGTGCAG
TGGCACGATCTCGGCTCACTGCAAGCTCCGCCTTCTGGGTTCACG
CCATTCTCCTGCCTCGGCCTCCCAAAGTGCTGGGATTACAGGCAC
GTGCCACCATGCCCGGCTAATTTTTTTGTATTTTTAGTAGAGACG
GGGTTTCACCGTCTTAGCCAGGCTGGTCTGGAACTCCTGACCTCA
TGATCCGCCCACCTCCGCCTCCCAAAGTGGTGGGATTACAGGCGT
GAACCACCGCGTCTGGCTGAACTTCAAAGTATTTTAATGCCACAG
TAGTTAAGCCATATGATACAGGGGCCAGAATATATGTATTGTTTG
CATTACATCAGGCTCTGTTAGTACTTTACAGCATACTGTGAGTAC
GAAC

In some embodiments, the intronic domain that promotes trans-splicing primarily in the liver comprises or consists of sequences from the gene ASGR2. In some embodiments, the sequences from gene ASGR2 comprise or consist of:

(SEQ ID NO: 764)
TGTGTAGTGTGGTGTATATGTGTGTGTTGTGTGTGTATGCATGTG
TGGTGGTGTGTGTGAGATGAGTCTGTGTGTGTGCGGTATGGTATA
TGTGTGTGTACGCATGTGATGTGTGTGGTGTGTCTCTTGGTGTGT
TTGTGTGTGTGTGATGGATCTGTGTGTGGTGTGGTCTATGTGTGG
TGTACATGTCTGTGGTGTGTGTGTGGTATGGTGTGTGGTGTGGTC
TATGCATGGTGTACGTGTCTGTGGGTGTATATGCTTCCCCAGCCA
CAGGCTGGAGCCTAGCAGGTGGGGACCACACCTGCCGGCAGCACT
TTCAGCCTGTCTGTCCTAGATCACGATGCCCTGCTCTTCCATCTG
AAGCACTTCCCCGTGGACCTGCGCTTCGTGGCCTGCCAGATGGAG
CTCCTCCACAGCAACGGTAGGGGTGGGGCCGTCTCCAGGCCCAGT
GTGCCTGCCCCTCCGGCTGGATCTCATCTCCCTGCCCCCTGTCCC
CTCA

In some embodiments, the intronic domain that promotes trans-splicing primarily in the liver comprises or consists of sequences from the gene TMEM220. In some embodiments, the sequences from gene TMEM220 comprise or consist of:

(SEQ ID NO: 765)
GTCAGGCGGCCGCGGTGGGAGGGGGACCCGCCCGGCTCCCAGCTC
CGCCCCCTGGCCTAGCGGGCCGTGTAACTGACTAGCGGGCAGAGT
CGCAGGGGCGCACAGTTAGTGGGGAGGACGGGACTTGGGCTTTCT
GCTTGGGGAGCCCCCACTTTCTCCCCCGTCTAAGAGACGCGGATG
TGTTCATGGAAGCGAGTCCCATACCAGGGCTCACACCCTGGAAGG
GGCTGGTTCCGCGCGTGGAACTTCCCAGGGGACCTCAGAAGAAGC
GGCCCTGGCTCCTGCGTTAACGTCCGGCCCGCGGACGACACTGAA
CATATCTCAGGGCCTAGGAGACGAAGCCGACCAAAACCCTTTATA
GCATCATTAAACTTTTTAGTCGGCTTGGGGACCCCCTTCATAATC
ATTTCCCTCATTTCTTGAGACTGGTGGGGGTGGAGGGCCCTTAAA
ATACCCGTTCTGGGTTGGCGAGTCCTCTCCAGAGCTCCCGTGAAG
GCTT

In some embodiments, the intronic domain that promotes trans-splicing primarily in the liver comprises or consists of sequences from the gene TMEM220. In some embodiments, the sequences from gene TMEM220 comprise or consist of:

(SEQ ID NO: 766)
TGAGCCGTATAAGATGTTAATATAACTGTCGTCACTCTAGAAATT
ACCATAATGCTTACAGGGATTTGAAAGTTCCTGAGCTTTGGAAGG
AAATGCATAAATACTAAACAGAACACACACACACACACACACACA
CACACACACACACACACACACATCTCTTTTTCTTTCTCTTTCTTA
TTTTTGTAAAAACAATTTTTATCCTTTTTTTTTTGAGGAGTCTCG
CTTTGTTGCCCAGGCTGGAGTGCAGTGGCACAATCTGGGCTCACT
GCAACCTCCGCCGCCCGGGTTCAAGCAATTCTCCTGCCTCAGCCT
CCTGAGTAGCTGGGATTACAGGCTCCCGCCACCACGCCCGACTAA
TTTTTTCGTATTTTTAGTAGAGATACGGGGTTTCACCATGTTGGC
CAGGCTGCTTTCGAACTCCTGACCTCAGGTGATCCGCCCACCTCG
GCCTCCCACAGTGCTGGGATTACAGGCGTGAGCCACTGCGCCCAG
CCCT

In some embodiments, the intronic domain that promotes trans-splicing primarily in the liver comprises or consists of sequences from the gene NCOR1. In some embodiments, the sequences from gene NCOR1 comprise or consist of:

(SEQ ID NO: 767)
TCTGAATTTTCAGAGTGAGAATTGTTGTCCTGTCCTTGAAGCAAA
TCACCTTCAAGTATTACAGTGTAATATTCACATTCTCTGGTAGAT
GAGAGACACAACTTTGTGTAGTGGATATTTCCAATCAGACGCTGG
CTAAGCCTTCACCTTGGCATTTGCCAGCTGTGTGGCCACAGGCAA
GTCACTTAACCTTTCATAGCTTTAGTTTTCTCATCTGTAAAATGA
AAATAATAGTACACGTCTACCTTGCTGTGGTTGTAAAGTTAAGGA
AGGAGCATTCGGTATTGTTATATAACAGGTGGCTCTGCATTATTT
TATGTATAATGTAGCACGTTATTACATATTAATTAGTGTATTAGT
ACTGTATATACACAGTAGATACTATGGAGGTACCTGTTTTCATTG
TTACATCACTGTTTTTATTTCAAGTGATGGATTTGAGTTGGTCCG
TTGCTGTTTTAGTTGGTGTGTGATACACTTTTACTTTCTTTAAAA
AAAG

In some embodiments, the intronic domain that promotes trans-splicing primarily in the liver comprises or consists of sequences from the gene NCOR1. In some embodiments, the sequences from gene NCOR1 comprise or consist of:

(SEQ ID NO: 768)
GGCATGCCTCACTGCACCCAGCTAAATTTTTGTATTTTTGGTACA
AAGATACAAAATACCAGCCAGGCTGGTCTTGAACTCCCGACCTCA
GGTGATCTGCCTGCCTCGGCCTCCCAAAGTGATGGGATTACAGGC
ATGAGCCACTGCAATGCCTTAAGCATTATTCTTGAAGATAACTTT
GATTAAAAAATGATAAAATCAATTCAACCAATAACATGACAGAAA
TAAGCATTACATTACTTGTTAAGAGATGGCAATTATTACATTGGT
TGTTTGCATATTTCGATTTATTTTCTTCCTATAGGTATCCTGCAC
CCCATGTAACATACCAATTGGAACCCCAGTGAGCGGCTATGCTCT
CTACCAGCGACACATTAAAGCAATGCATGAGTCAGCACTCCTGGA
GGAGCAGCGGCAGAGACAAGAACAGATAGATTTGGAATGTAGAAG
TTCTACAAGTCCATGTGGCACATCCAAGAGTCCAAACAGAGAGTG
GGAA

In some embodiments, the intronic domain that promotes trans-splicing primarily in the liver comprises or consists of sequences from the gene UBB. In some embodiments, the sequences from gene UBB comprise or consist of:

(SEQ ID NO: 769)
TAAACTGCCTCTCGGGAGGTTGGGCGCGGCGAACTACTTTCAGCG
GCGCACGGAGACGGCGTCTACGTGAGGGGTGATAAGTGACGCAAC
ACTCGTTGCATAAATTTGCGCTCCGCCAGCCCGGAGCATTTAGGG
GCGGTTGGCTTTGTTGGGTGAGCTTGTTTGTGTCCCTGTGGGTGG
ACGTGGTTGGTGATTGGCAGGATCCTGGTATCCGCTAACAGGTAC
TGGCCCGCAGCCGTAACGACCTTGGGGGGGTGTGAGAGGGGGGAA
TGGGTGAGGTCAAGGTGGAGGCTTCTTGGGGTTGGGTGGGCCGCT
GAGGGGAGGGCGTGGGGGAGGGGAGGGCGAGGTGACGCGGCGCTG
GGCCTTTCCGGGACAGTGGGCCTTGTTGACCTGAGGGGGGCGAGG
GCGGTTGGCGCGCGCGCGCGTTGACGGAAACTAACGGACGCCTAA
CCGATCGGCGATTCTGTCGAGTTTACTTCGCGGGGAAGGCGGAAA
AGAG

In some embodiments, the intronic domain that promotes trans-splicing primarily in the liver comprises or consists of sequences from the gene UBB. In some embodiments, the sequences from gene UBB comprise or consist of:

(SEQ ID NO: 770)
GTTTGTGTGGTTTCTGGAAGCCTTTACTTTGGAATCCCAGTGTGA
GAAAGGTGCCCCTTCTTGTGTTTCAATGGGATTTTTATTTCGCGA
GTCTTGTGGGTTTGGTTTTGTTTTCAGTTTGCCTAACACCGTGCT
TAGGTTTGAGGCAGATTGGAGTTCGGTCGGGGGAGTTTGAATATC
CGGAACAGTTAGTGGGGAAAGCTGTGGACGCTTGGTAAGAGAGCG
CTCTGGATTTTCCGCTGTTGACGTTGAAACCTTGAATGACGAATT
TCGTATTAAGTGACTTAGCCTTGTAAAATTGAGGGGAGGCTTGCG
GAATATTAACGTATTTAAGGCATTTTGAAGGAATAGTTGCTAATT
TTGAAGAATATTAGGTGTAAAAGCAAGAAATACAATGATCCTGAG
GTGACACGCTTATGTTTTACTTTTAAACTAGGTCAAAATGCAGAT
CTTCGTGAAAACCCTTACCGGCAAGACCATCACCCTTGAGGTGGA
GCCC

In some embodiments, the intronic domain that promotes trans-splicing primarily in the liver comprises or consists of sequences from the gene LRRC75A-AS1. In some embodiments, the sequences from gene LRRC75A-AS1 comprise or consist of:

(SEQ ID NO: 771)
CCTGAGGTCAGGAGTTCAAGACCAGCCTGGCTGGCCGACATGGTG
AAACCCCATCTCTACCAAAAATACAAAAATTAGCCGGGTGTGGTG
GCGTGTACTTGTAATCCCAGCTACTTTGGGGGCTGAGCCAGGAGA
ATCGCTTGAACCCGGGAGGCGGAGGTTGTAGTGAGTCGAAATCGT
GCCACTGCACTCCAGCCTGGGCAACAGCGAGACTCCATCTCAAAA
AAAAAAAAAAAATTACAACCTACTGAGAAGCTAATTAAGATTTGT
AGTCCTCAATAATTTACAAAAACTTAGAAAAATTACCTCTAATTA
GGTCTTAAGGGGCTTTTGTTAGGATGGACTTAAATTTGTAAAGTC
ATTAATTGACTGTCATCAGAAATGCCTGTCCTGCTCTGCATCTGC
AGTCTCTAAGGTGTATGCGCCTTCTCCATTCCCCCAGGTTTTGAG
AACAGAGTGCTGTTTTAGAGCTGGCAGCAGCATCTCAGCCCAAGA
GAAG

In some embodiments, the intronic domain that promotes trans-splicing primarily in the liver comprises or consists of sequences from the gene LRRC75A-AS1. In some embodiments, the sequences from gene LRRC75A-AS1 comprise or consist of:

(SEQ ID NO: 772)
AAAAAAAGATATCAAATGATGAAATCACCCAAAATAGCTGGAATT
ACCGGCAGATTGTGTAGTGGTGAACCTATGGTTTTCTGAAGATAT
CTCTTACCCTGTGATTGACTGTCAGTTGGTGCAGAAAGTGAGATT
GAAATGTAGTTTCTTTGCAGGTTATATTCCCAGAGGATGTCAGTC
CCAAGGACCAGTAGCTGCCATCAGTTTGGATTCTGAAAACTAACT
GGCATCAACACTGGGTGTAGAAACATGCTTGCCTTATGTATCAGA
GGACATGCTCAGCAGATCCAAGAGATATATTTGGCAACTTTTTCT
AGAAAAGGCACATTGGGTATCATTCATTACATTCTTGAGTTTTTT
TGGGTTTTTTTTTTTTTTTTTGAGACAGTCTTGCTGTATTGCCCA
GGCTGGAGTGTGGTGGCACAATCACAGCTCATTGCATCCTCAATC
ACCCAGGCCTAAGCAATCCTCCCACCTTGTAGCTGGGACTACAGC
TCAC

In some embodiments, the intronic domain that promotes trans-splicing primarily in the liver comprises or consists of sequences from the gene SKAP1. In some embodiments, the sequences from gene SKAP1 comprise or consist of:

(SEQ ID NO: 773)
GTAAGGACAGAAAGTGTGGGGCTTAAATAGAAAGTCAATTTTTTC
ATTCTTGATTTAGTCACCATAAACAGAATTATTCCATGTCATACA
CTTCCCATGGCTCCCTCACAATATAAATTCCCATTTATTCATAAT
TGTTAGGAAAAGTTTTAAATGAAAATCCAGATTCAGGAACCATTT
TCACATTTAAAAATACAAGGCTAGGCCGGGCGTGGTGGCTCACGC
CTGCAATCCTAGCACTTTGGGAGGCCGAGGCTGGTGAACTGCCTC
AGCTCAGGAGTTCAAGACCAGCCTGGGCAACATGCTGAAACCCAG
TCTCTACTAAACTACAGAAAATTAACCAGGTGTGGTAGCACATGC
CTGTAGTCCCAGCTACTTGGGAGGCTGAGGCATGAGAATTGCTTG
AACCCGGGAGGCGGAGCTTGCAGTGAGCTAAGATCACGCCACTGC
ACTCCAGCCTGGCGATGGAGCAAGACTCCATCTCAAAAATAAATA
AATA

In some embodiments, the intronic domain that promotes trans-splicing primarily in the liver comprises or consists of sequences from the gene SKAP1. In some embodiments, the sequences from gene SKAP1 comprise or consist of:

(SEQ ID NO: 774)
CAAAGTTCCTATGGACAAAGAAGGCCGAAAAGTGAGCTGTGATCC
AGAAGCCTGCTGGCTGACTAAGCCTTTCTTGTAAATAGGCAGAAG
TCACAGATTTACTCTGATCCTTCAAATGCATAATGAAGAGAAAAA
CTAGATAGTTAGTTGCCCTGGATCAGGAAAGTCTGGCGGGAAACC
TCTGCACTGCTTACTTACAAACCAAGTGAAAAATTACCTGCCTGA
TTTTCTTTTATTAATAAATAAATATATTTATCATATTGTTGAACG
GCATCCTACTAGGAGTGATCTGTAAATATTTAATGTTAGTTAATG
ATCTCCTTCTCCCATAGTCCTTTGCTCATGCTGAGTGTTTGGCTG
TATCTCATCTCAACTCCCCTTAATCTGAGGTTTCCTAGGGGCAAT
ATCAGAACCCTGGTTTCCATTTGCAGCAAAAACATGGGTACCTCG
GAAAAAATAGAGTCCTTTTAACTTTGATTTCTCCTCTCTTTGATC
AGTA

In some embodiments, the intronic domain that promotes trans-splicing primarily in the liver comprises or consists of sequences from the gene TOM1L1. In some embodiments, the sequences from gene TOM1L1 comprise or consist of:

(SEQ ID NO: 775)
ATTCACCAACAGTTCAGTGATACTTGAATTCTGGTCTTCTTTCCC
AATCAGGCTGCTCCTATTATTCAGAGTCCTCAAATACTTGCTCTA
TGCATTCCGTCCACCTTTAAGTGACATTCAGTGGAGAGACAGAGT
GCAGTACGTTTGCTCCGCTTTATCCTGGACCGGAGACCTCACAGC
TTTCTTCTTAACCTGTGTTCTATACTACTTCTCATCCTTTAAGAA
ACGATGGGGTGGTAGCTACACTGATGGAAAGACTAAAGGCTCTGG
TCTTAGTGTTCCGTGCACACTCCACAGTCTCAGGGCTCCCACCAC
CCATGGCATCATAACCTGATGTGCACTAAAACCAATTGTTTTATA
AACAGTTATTACAAAATGACACTTTATGCAGTCTGAAGGTACCTT
TTAGGTTTTGTTTTTAGACTAGGAAAATTATCTTACTTTTTGCAT
TTGTGTTCTCACTGTAGGGTAATCATTCTGTGTTATCACACTATC
CTAG

In some embodiments, the intronic domain that promotes trans-splicing primarily in the liver comprises or consists of sequences from the gene TOM1L1. In some embodiments, the sequences from gene TOM1L1 comprise or consist of:

(SEQ ID NO: 776)
TAAGTAACAAAACTCTTTTCTAGCAGTGCATCTAGTCAGCAGATT
ATTGTAGCCAGAAAAGAACTCATGGTCATTAATTCCCATGTTCTG
CTCAGTATTGTGTTGATGCGCAGCCCCTGTTAGCAAGATGGTAAG
GATCAAAATTTTTTTGGAGTTTGTATTTGCTATCTCCACTTCCTT
TCCTTGTATTCTCTTTGACTTCAGACATGCTCTTCTACCACTCTA
TGGACATAACTCTTGTCAAGGTCACTGGTGACTTCTGTATTGTGA
AATTTAGAACTGCAGTTCCATAGTCCCTCATTTTATCAGCAACAA
ATGACAAAGTTGATCACTTCCTCCTTCTTGCACACATGGCTTTCA
GACACCAGTCACTTTGGTTCTTCTCCTGCCTCACGTTCATTTCTT
CTCCATCTCTTTTGGTGGCTCTTCCTCCTCTACTGACCTTTTCTT
GTTGGAGACCCCAGGGATCAGGCTTCAGACTTTTCTATCTACATT
CATT

In some embodiments, the intronic domain that promotes trans-splicing primarily in the liver comprises or consists of sequences from the gene ABCA6. In some embodiments, the sequences from gene ABCA6 comprise or consist of:

(SEQ ID NO: 777)
GTAAGTTGATACATTTATTACCACTTTCTCTGCTTTAGATCTTAG
CTATATGTAGCTAATATTAATGCCCTAGTAGGTTACTGCAATCAC
TACTTTCATGTCCATTGTGTGTGTGCATGTGTGTGTCCTTGTGTG
TACTTGAAATAGTTTCTATGGAGTGAAAAAGTTATGTAATCCAAA
TAAAATTTGACTTGCCTTTTGCATAAAGGTCTGAACTAATGAAAT
GATGTATCAAAAAGAAAAAACAATGCTGACTCTAAAATATCAAAG
TCTTTTCTGTATAATTATTGAGGTTATTTTTGAGGGGTGGACCCA
ATTTTTTTTCCCTTAAGGGTGCTCATCACATGGTAGCTGATCACA
ATATCTCTTCTCCCTCTAATATTCTGTCTCATACTTACTTCTCGT
TTCACTCCGCTCTGCCTTCTCTGAACAAGAACCTCTAATTTCCAT
CTTCCAGGTTGTTTTCTGCAGAAAGTATGTAGATGCTCCTTCTTA
CATT

In some embodiments, the intronic domain that promotes trans-splicing primarily in the liver comprises or consists of sequences from the gene ABCA6. In some embodiments, the sequences from gene ABCA6 comprise or consist of:

(SEQ ID NO: 778)
TCTGCCTTCTAGGTTCAAGCGATTTTTCTGCCTCAGCCTCCCGAG
TAGCTGGGACTACAGGCATGCACCACCATGCCTGGCTAATTTTTT
TGCATTTTTAGTAGAGACGGGTTTCACCATGTTGGTCGGGCTGGT
CTGGAACTCTTGACCTCAAATGATCTGCCCACCTCAGCCTCCCAA
AGTGCTGAAATTACAGGCATGAGCCACCGTGCCTGGCCTTGGCAT
CCTTTCTATTGGTTGGTGCCTATACCAGTGTTCAGCATCTATCAT
CTCTGTATGCATATATGGTAAAGGTAACGAAGATGAATGGCAATT
ATAAAGAAGGAGAATTGCGATTGAAAAAGGATATGCAATGAGCTT
CTGCTGTAGGATTAACAGTATAGTTATTAATCTAGAAGGTACAAG
GGTAGTCATTACATTGTTCTTTAGATGCTTTTGTATATATTAAAT
ATTTTGTAAATAAGTTAACATAATTTATATAACTTTTTGTCTTTA
TTTA

In some embodiments, the intronic domain that promotes trans-splicing primarily in the liver comprises or consists of sequences from the gene AFMID. In some embodiments, the sequences from gene AFMID comprise or consist of:

(SEQ ID NO: 779)
GCTTGGGAGGCTGAGGCAGGAGGATCGCTTGAGCCCAGGAGGTCG
AGGCTACAGTGATCCATGATCGCACCATTGCACTCCAGCCTGGGC
GACAGAGCGAGGCCCTATCGCTTAGAAAACAAAAAGTTACATGAA
GTTCCCCCAAGGTCCCGTAGCTAGCATGTGATGTGGCAGGGAGAG
AGCCTGTCCGCTTCACTCTCTCACATGGGACCAGAAAGGGGTGAT
GCTGTGCGTGGACAGGACATCTGGCAAGTGGTGTGTCCAGGACAC
AGGAAGCAGCGTAGTGGCCAAGCTGCCTCCAGCTGTCACATCTGT
GTGTCTCTGCAGTAAGGATGAGTCTGCCTTCATGGTCCACCCGCT
GACGGCACAGGGAGTGGCCGTGGTAATAGTGGCTTACGGCATCGC
CCCCAAAGGTAATAGGAGTGGTTGCTGCAGGTCCGAGGGCCGGTG
GGCTTTAGGAGGAAGTGCATCCCTGACCCAGCTCATGCTCTCTGT
GCAG

In some embodiments, the intronic domain that promotes trans-splicing primarily in the liver comprises or consists of sequences from the gene AFMID. In some embodiments, the sequences from gene AFMID comprise or consist of:

(SEQ ID NO: 780)
TGGGTGTTGCCAGTAGATTTTCTTCCTGTTGGACCTCAGTGGGTG
GGAGGACAGTGCAATCAGTACCTAGGATACAGCCAAGCTGCCCCT
CTGGCCTGTGGAGCAGAAAGCAAATTGGGACTCTTCGAAGGGGGC
CTGATGTTGTGGGGAAACTGCGTGCAAATCCAGCTCTCTGCTCTG
ACCCCTCCCAGGGGAATTTACCTGTGTGGACACTCAGCCGGGGCC
CACCTGGCTGCCATGATGCTCCTGGCCGACTGGACCAAGCATGGG
GTCACGCCCAACCTCAGAGGTTTCCATGGGAGCTACAGCCTGGCT
GGGCAACCTTCATCTCCCCATGAGCCTTGGGGTTTGGGCCAACTG
CTTGAAATCCTCCCGGCCATGAGTGGCTTGACCGCAGGGCTTCGA
GCCCTCTGAGCAAGGCCTTCTCTGCCTCCTCTGTGCCCCTTCCCC
TGGTCCTGCCCCTCTGGCGGTGGGGGTGGGCTGGCCCTGCCTTGC
TCCG

In some embodiments, the intronic domain that promotes trans-splicing primarily in the liver comprises or consists of sequences from the gene TTC39C. In some embodiments, the sequences from gene TTC39C comprise or consist of:

(SEQ ID NO: 781)
AAGAACTGTTGATCCCAAAAGGGAAAAATTAGACATAGGGTGTGA
CGTGTCTAGATACCTCCACCTGCGCTCATGCACACATTCCTGCGC
CATAAAGGATTGATTTTTTTTTTTTAACCTTGGTGTTTTGTGTTT
GAGCTGTGTGCTAACAGTCAGTGTCAATGTCTGCCTTCTGAGCTG
TGGGCGATTCCCATTGTTGTGAGGTTTCCTCCAACTGGACGTTAA
GTTACTGAGAACAGGAATCAGGGATGCGATTGGTGCGTGTCTTCT
GTGGCTACCTGGTCAGCATTCATAAACGGTGATAATGGATTGAAT
GCTGCAGTTTCAACCTTCCTGACAACAGAGGAGAGTGTTACTTTG
GGGCATGAACAGTTATTGATCCAGCTGGGCCTTGGTTAATGAGTC
ATTTGGCTTACAGTGTTCCCGAGTCACAGGCTGTCCTGAAGATCA
CCTGCTGACTCATTGGTGCCCTTGTTTCCCCGAGGCCTCCTGCAG
TGAC

In some embodiments, the intronic domain that promotes trans-splicing primarily in the liver comprises or consists of sequences from the gene TTC39C. In some embodiments, the sequences from gene TTC39C comprise or consist of:

(SEQ ID NO: 782)
TATGTGACATTTAGCATTTCTCAATGCCTAAGATTAAACTGATGC
TTTGAATCCTGATCATGCTTTTGAGCTGCTCCTCTTGGGTACTTG
TGGCATCGTGTTGGCAGACCCTGAGTGACATCTTCAGCGGCGGGA
AAACGAAATGTATTTCACTGGGAGTAAGTAGAGCGGACATTTGAG
AGTTTGCTGGGTCCCAGACAGTGTTATGTGAATGAAAGTGTTGGA
AATGTAATCTGGATCCTCAGGGAATTTCTGAAGTGTCGGGAGAGG
AGATTTGATGAGCAGAAGCAATTGGAGAACAGCATTCTAGGGTAT
AGAAGCAAACTATCAACTGTATGGTGAAGAGCTCACTGAGAACTG
GGCTAATGGAGCGGCTTCGTGCATTCACTCAGTAGCGATTGAGAG
ACTTCTGGGCCAGGGCTGGGCGCACACGGGGATGAAATATGGTCC
CTTCCCTTTCCACACCTGTGTGGTAACAAGGGGCTAGATTAGCAA
ACAA

In some embodiments, the intronic domain that promotes trans-splicing primarily in the liver comprises or consists of sequences from the gene MOCOS. In some embodiments, the sequences from gene MOCOS comprise or consist of:

(SEQ ID NO: 783)
GTTCAGTACTGAAGACCTAAACATTTGTCACAAGGGCAGTACCAT
CCACTATTTACACACATTTAAAATTATTAATGTGAGGTCCAGGTT
TGCCAGTTATAACAAACTTGTTTTCTTAAAAGTGAGTTAGTGGTG
CCAGGCGTGGTGGCTCATGCCTGTAATCCCAGCACTTTGGGAGGC
TGAGGCAGGTGGATTACTTGAGCCCAGGAGCTCAAGACAAGCCTG
GGCAACATGGTGAAACTCCATCTCTGCAAAAAATACAAAAATTAG
TCAGGCATGGTGGTGAATGCCTGTCGTCCCAGCTACTTGGGAGGC
TGAGGTGAGAGGATCACCTGAGCCCAGGAGGTTGAGGCTGCAGTG
AGCTGTGATCATACCACTGCACTCCAGCCTGGGTGACAGAGTGAG
TGAGACCGTGTCTCAAAAAAAAAAAAAAAAAAAAGAGTGAATTGA
GAATTTACATAAAGCTCATGATTCTCTTGTGTTTCATGATTGATT
TGAG

In some embodiments, the intronic domain that promotes trans-splicing primarily in the liver comprises or consists of sequences from the gene MOCOS. In some embodiments, the sequences from gene MOCOS comprise or consist of:

(SEQ ID NO: 784)
TGGGTTTTCTTTCCATCCTGCTGACTTTATTACAACTCATCCTAGT
TCCAGACGAGAGCAATGTAGTGTGACAAAGTCCATGCACTGTTGA
AGAGGCCACCAGGCCCTCAGCCACATGCCTTTCCACCTTCATTCA
CCCTCGTTTTTCAGTCCTCTTTACTCAGCCAAAAGTGGTAACTTT
CAAATGGGAAGAAAGAGGCCAGTTCTTCTGATGGCAGGTGGGAAA
CTGTTGGGTCTTTGCCATAGTAGAATAAGCTGTTCACTTCACTTC
CTGGTCCACTAAAATGTCCAGAGTTGCACTGAGCACTCAGGCATG
GTGGTAGGTGGTTATCAGACGGGGTGTCACAGATGGACAACAGGA
ACCAAATGTTACTCCCAGAGCAGCACTGTGGGGAATGGATTCAAT
CGTTGGGTGTTTGGAAGACGGTGCTGGTTTTTCCTTGAAAGCACA
TCTGCATCCCCATTTTTATTTATTTTTTTGAGGTGGGGTCTCGCT
CTT

In some embodiments, the intronic domain that promotes trans-splicing primarily in the liver comprises or consists of sequences from the gene PET100. In some embodiments, the sequences from gene PET100 comprise or consist of:

(SEQ ID NO: 785)
CTAATGTCTGCCTCCCATAAGCCGACCCTGCCCTGATGTGGGGCT
CTGGAGGCCTTTGGCTCGTGTCCCATTTGTGACTTTTCCTTACAG
AGGGAGCTGTGGCCACCTGAGAAGGTAAGTGATCTCTTCTTCCTG
CCAGAGGGATGGAGGAGGCTGGATTCTTGTATCCGGGATGGGTTT
TAGATGAAAATTTTAGGCCAGGCGCAGTGGCTCATGCTTGTAATC
CCAGGGCATGGGAGGCTGAGGTGGGTAGATCACTTGAGGTCAGGA
GTTCAAAACCAGCCTGGCCAACATGGCGAAACCCCATGTCTACAG
AAATACAAAACTTAGCCAGGCATGGTGGCAGGCGCCTATAATTCC
AGCTGCTTGGGAGGCTGAGGGAGAAGAATCACTTGAACCCAGGAG
GTGGAGGTTGCAGTGAGCCCAAGATCACGCCACTGCACTCCAGCC
TGGGCAATAGAGTGAGACTTTGTCTCAAAAAGAAACATTTTACTC
TCAG

In some embodiments, the intronic domain that promotes trans-splicing primarily in the liver comprises or consists of sequences from the gene PET100. In some embodiments, the sequences from gene PET100 comprise or consist of:

(SEQ ID NO: 786)
TAATTGGCAGGGGGCTTCCAGTGAAGACACTGAACCCTGAAGCCG
TGGTCTGGGGCAGGGTGGTGGGAGAGGTGGGCGGGGGGGGGTGGT
CCCGGCTCTGAGGTGTGTGCCCCTCCCCCCTTCCTGTCCCACCCG
CTTCTCCACCCCTAGCTTCAAGAGATAGAGGAATTCAAAGAGAGG
TTACGGAAGCGGCGGGAGGAGAAGCTCCTTCGCGACGCCCAGCAG
AACTCCTGAGGCCTCCAAGTGGGAGTCCTAGCCCCTCCCCTGATG
AAATATACATATACTCAGTTCCTTGTTATTCATTTAAGTGTTTTA
TTCTTTTATCAGTTTTTGGGGGCCGAGTGAGACCCAGGATGCCTC
AGGCCCTCAGGGGGCTTGTGTCGGGGGCTGGGGGCTGCTGTTCCG
ACGGAAGCCGAGAGCGGTCGGGTCCTGAGGGGTGAGCAGGGGTTG
GGGACTGAGGGTCCCAGCTCGTTTCTGTGCTCCGTCCTGTGGATG
AAGA

In some embodiments, the intronic domain that promotes trans-splicing primarily in the liver comprises or consists of sequences from the gene PRKACA. In some embodiments, the sequences from gene PRKACA comprise or consist of:

(SEQ ID NO: 787)
GTGAGGCTGAGTCCTGTTACTATAGCAACTTCCTAGGCACACTCTGCCC
CTTAAGTCAGGGAGGGGCTGCAGCGGGGTCTGGGGCACAGGCGAAGAGA
AGAAGGATGTGAACCCCTGGCTGGCCCTGGAAACGCCTCCTGTACAGCC
CCGGGCTGTGCGGCCTGGGGAAGAGAGAGGGGATCGGTGGCCCAGTTCA
GACAAGGGAGGATGGTCCACCCGGCAGGGGGCCAGCTCAGAGAGGGGTG
GTGGAGTGGTCCATCCCAGTGTTGGGGTTCAGCTCAGACTGTAGGATGA
GCCATCCCAGTGTTGGGGTCCAGCTCAGAATGGGGGTCGTTCCAGCCAG
TGTTGGGGTCCAGCTCAGAGTTGTGTCATTTAGGTAAGCCCAGAGCAGG
GAATCCAGGCTCCACTGGAGGAATCAGCCAGCCTCGAGTAGGGGGCCCA
GCCCAGGCTGGGCATTCCTGCCCCAGAATTCCCAGGACAGCCAGATTTC
AAAAGGGCA

In some embodiments, the intronic domain that promotes trans-splicing primarily in the liver comprises or consists of sequences from the gene PRKACA. In some embodiments, the sequences from gene PRKACA comprise or consist of:

(SEQ ID NO: 788)
CAGCGGCCCCAGGGGGCAGGCGCCCAGGTCAGACAGCCAAGTGGGGCTC
AGGTCGTGGCTGGCACTGGCTGAAGATACCCCTGGGAGCTGGCCCACAG
GGGAAGGGGCAGAGCTAGTAAAAGGGAGCCCCCGATCCCCAGGACACAA
GCAGATCCTGGCAAAGAGAGAAAAGCACCCCCCAAGAAGACCCTAGATG
GGAAGAACTAGTGCCGTGTAACATCCTATGGATGCCTAAGACGGCCAAA
CACCTTCTGTGCCTTGGCAGTTGTATGGGTGCCCAAGAGGTCTTATCAA
GGCAGCTCTCCCCTTAGCGCAACCGCGTCTACAGAAACTCGGAGGGCCC
TATCTGTCCCTCCTCCACACCCAGTGGCCTCTGGGTTGGGTTTCTCTTC
CTGCTCCCACCCCACGGCTCCCTAGCTCCCCCTGCAGGCAGGGTTCTGG
GGACAGACAGCCGAACAGACACGGCAGGTCTCATGAGCCTTCCCAGCCA
CCGTAGTGC

In some embodiments, the intronic domain that promotes trans-splicing primarily in the liver comprises or consists of sequences from the gene SLC27A5. In some embodiments, the sequences from gene SLC27A5 comprise or consist of:

(SEQ ID NO: 789)
GTCCGAGGCAGGGTTTCTCGGGGCGGGGCTGGCGCGGGGCCACGGACGC
TTTCAGATCCTAGAGGGAGTCAGGCTGAAGAGGCGGGACTTTCCTGTCG
TAAGAGGGCGGAACTTGGAGTCAGGACTTCTCACGCGAAGGCGGGGCTT
CTCGATTTTCGAGGGGGCGGGGCTGACTGGGCCTCCTGATCCCCAAAAG
GGGCGGGGCGGGGGCAAGAGGCGGGGCCTCTCCTTCCCGGGGGCGTGGT
CACAAGCGCCGGCTCCCTCCTTGCAGATGGAAGGGCGAGAACGTGTCCA
CGCACGAGGTGGAGGGCGTGTTGTCGCAGGTGGACTTCTTGCAACAGGT
TAACGTGTATGGCGTGTGCGTGCCAGGTGCGGAGGGTTCTCATTTTAAA
CTTCCAACAGCTTTGTGGGTGGAGGGGTCGTTATTCTGATTTTACAGGT
GGGGAAGTGCTAGGGCCCAGAGAGGTTTAGAGACTTGCTGGCTCTCACA
GCCAGTGAG

In some embodiments, the intronic domain that promotes trans-splicing primarily in the liver comprises or consists of sequences from the gene SLC27A5. In some embodiments, the sequences from gene SLC27A5 comprise or consist of:

(SEQ ID NO: 790)
TTACCACCATCTGGGGCCTCCGCCTCAGCTTCTCGGCTCTGTCCAGCTC
CCCTGCCTTGATCTCTTGCTCTGATTTACTGGGTCTCTGGGTGTCCATC
TGATTTGTTCTCTTGAGCTTTCTGTGGAAGACGGCATTCCAGCCATTGG
TCCCTTGTCCTGTACCTTCTGTTAGTGTCTTCTCCCAGGGCCTTGCAAG
GTCCTGGTCATATCTATTCTCACTGCCATTTGTCTGTCCGTCTTCCATC
CCTTGTTATCCTTTTCCTGTTTCTCATGGGCTCCCCATACCGCCTTCTC
TATACTTCTCTCTCATTTTCTGTGTCGCTTGTCGCAGTCTCTCAAGGTC
TCTGAATGTCTCTCTCATCTCTGGATCTCCCTGTCTGTGTCTTTGGGTC
TGTCCTTGGCAGGGTGCTGGGTGATGAAGAGTCCACCGTTGTTGAAAAC
CTCCAGCAGGGGGCTTGGCTGGTGGGCTCCAGGGGTTCTTGTTCCTGCT
CCTACCCCA

In some embodiments, the intronic domain that promotes trans-splicing primarily in the liver comprises or consists of sequences from the gene ACSS2. In some embodiments, the sequences from gene ACSS2 comprise or consist of:

(SEQ ID NO: 791)
CACTTGATGAGGTCTGCACCTTCTCAGTGATGTCCACTCCCACAGTGTA
GTTGGCAGCTCCTCACTGGAGTTTGAGTCCTCTGGTAGGGTCCACGTCT
CCTTAGTAAAGTCTATATCCTCTGTATCTCTTAGGGGAAATCCATGTCT
CCTCACTAGGGTAAGTGCCCTTCAAAGCCATTCATGTCCTATTAGCAGG
GCCCAGGGCCCTTCAGTCAGATCTGAGAACCCCCAGCAACATCTGTGCT
CCCTTAGTGGAGTCCATGTACTGTCAGTGAAGTGTGTGTCCCTTCGGTG
CAGTGAATATCTCCTTTATGAAAGTATCTGACCCCTTGGTGGGGTCTAT
GTCCACCCCAAGAGGTCTGTACTCCTTCAGTGGGCCCTCAGATTCCCGC
CCTCCTTGGGATTGGGCAGACTCTCGGTCTCCCATAGAGAGATTTTTTT
TCAGCTTAGGGTGTGCAAGGTTTACAGTCAGCTGGGCAAAGTGCAGGGA
AGTAGCTGG

In some embodiments, the intronic domain that promotes trans-splicing primarily in the liver comprises or consists of sequences from the gene ACSS2. In some embodiments, the sequences from gene ACSS2 comprise or consist of:

(SEQ ID NO: 792)
TGAGAGATGGAGGAGGGTCCTGCCTGACTTCTTTCTGGGGTGGAGGGGC
TAGAGAGAAGCTAGAGGTAATTAAGCCCAGGTTGTGGTGGTGGTGGTGG
GGGTTTCCCTTGGTGGAGTAAAGAGATTGTGAAGGAAAGAAGAGAATGT
GGGCCAGGTGTGGTGGCTCACGCCTGTAATCCTAGCACTTTGGGAGCCC
AAGGCGGGCAGATCACCTGAGGTCAGGAGTTCAAGACCAGCCTGGACAA
CATGGTGAAATCCCATCTCTACTAAAAATACAAAAATTAGCTGGGTGTG
GTGGTGTGCACCTGTAATTCCAGCTACTCGGGAGGCTGAGGCAGGAGAA
TTGCTTGAATCCAGGAGGCAGAGGTTGCAGTCAGCCGAGATTGTGCCAT
TGCACTCCAGCCTGGGCGACAGAGCGAGAGACTCCATCTCAAAACAAAA
ACAACAACAAAAAAAGAGAATGTGAATGATTTTTCCTATTTGCACAGAC
TTTGATATT

In some embodiments, the intronic domain that promotes trans-splicing primarily in the liver comprises or consists of sequences from the gene LBP. In some embodiments, the sequences from gene LBP comprise or consist of:

(SEQ ID NO: 793)
CCAAGGCAGAAAATCGCTTGAGCTCAGGAGTTCAAGACCAGCCTGGGCA
ACGTGGTAAAACCTGTCTCCACAAAAAAATACAAAAATTAGCCAGGCAT
GATGGCACATGCCTGTAGTCTCAGCTACTTCAGGGGCTGAGTTGGGAGG
ATGGCTTCAGGCCAGGCGACAGAGGTTGCAGTGAGCTGAGATTGTGCCA
CTGCACTCCAGCCTGGGTGACAAGGTGAGATCCTGTCCCCCACCCCCCC
GCAAAAGAAAAGAAAAATGTGGATTCTCAAGCCGCTTCCCCAAATCTGC
TGAATGTGATCATGCGATTGGGTGCCGCAGGCACTGGGAAGCCAGACAC
ACCGAATCAGGCTGGATGTGCTGGGACACAGCAGGGCGCCGGGAAGGGG
TGGAGAGGGCTCCCTTTGTGGACTGGCCTGACCAGGGACCAGGGCCTGG
CTTTCCCAGGCACCCGGCCATCCCCAAGTTCAGTGGCAGACTGTGCTTC
TCTCCCCAG

In some embodiments, the intronic domain that promotes trans-splicing primarily in the liver comprises or consists of sequences from the gene LBP. In some embodiments, the sequences from gene LBP comprise or consist of:

(SEQ ID NO: 794)
TAAGAAGGTCCAAGCCTCTGGCCTCCCCCGAGCTTGGCGAGGGCTGAAT
GGAAGACCTCACTGACCAATGGCCCTGGACCAGGCCATGGGGGCTGGTT
TAGACGTACTTGGGCCCAAAGCCCAGGGAGGACAAATCATTTGCCCAGG
TCATGGAGCTGTTGTGGGGCACGCTGGGGACAAAGCTCCAAGTCCGTTC
GGTTGGGCAGAAAATGGTCAACTGCTTGACCAAGGGTTAAACAGCAGGG
GCTAAACAGAGACTCCAGCCCCTCCCTCTGCTCCACCTTGCTCAGTGCC
CCACACTGCCCTGCATTCCACCAAGGTCCGAAAGGAAACCATTCCTCAG
GCGCCTGCAAGAGACCAGGCAGGAAAGCTCAGCGAGGGCGTGGGCCAGG
TGGGAACTGGGCGAGCTCATGAGCACCAGCCCTGGATGGGGAGGCAGCA
ACACTGTCAGACTGGCCTGTTAAAAAGGAGTATGTGAAATCTTCAGAGT
GTATCATCT

In some embodiments, the intronic domain that promotes trans-splicing primarily in the liver comprises or consists of sequences from the gene PABPC1L. In some embodiments, the sequences from gene PABPC1L comprise or consist of:

(SEQ ID NO: 795)
AATCCCTCCATGCCAAGGTAAGCAGGAGCCTGGGCAGCAGGGAGCCCGA
GGGGGCAGGAGGAGAGCTAACGACAAGGGCTCTCCTAGTGGAGCCAGTG
GATAGCTGTTTGGCCAGATAAAGTGTTTCTTCTTGCTAATACCTGAAGG
CCTGCTGAATCTTAAGAGTGGAAGGGGCCTTAGGTAAGGGTTTGCTACT
TCAGCCCTTCCGTAGACTAATTCTGTGTTGTTCTGTAGGTAGGTCTTTT
GGTACAAGGAAGTGTGTTGTGACTCAGTGTAAGGGTTAAGATTTTTCCA
GCTGTCCAGCAATGTACCTCTGTGTCAAAGGTGACAGACAGGGCCATGG
GATCTTGCAAGCAGTGGTGGATGGGAGGGTTCCACAGAAACCCTGTGGA
TGGAGGAGGATCCTGTATCCAGGATGGTGAGCGAGGGGGAGATCAAGGC
TCTTCTGTGACCTGCTAAGATAGCTGCACTAAGCCCCCCCGCCACTCAT
GTCTCACAG

In some embodiments, the intronic domain that promotes trans-splicing primarily in the liver comprises or consists of sequences from the gene PABPC1L. In some embodiments, the sequences from gene PABPC1L comprise or consist of:

(SEQ ID NO: 796)
TGGGTGGAATGGTGACAGAAGCAGCTGAGCCCGGGAGAAGCTGTAAGGA
AATAGGCAGGGACTGACTTTCACAAACACTGAGAATTGCGCCGTGTTCC
TGGCTTTCTCTGAAGCACCAAAGAGTTTGGAGGAAGTTGTGAGAATCAG
AGGAGATGATTAACTGTGATCGGAATGGGGGGTGGTGGAGGGTGAAGGA
GGTGCCCTCTTGCCAGCTGCCCTGCCTGGCTTCATGCAGAATAGTTTGG
GCTTTCCCAGGGCCCTGCCTGGCTCCATTCTGCCTGCCTGAAGGCCTGG
CCAGGATGTAACCACCTTCTTTATTGAAGACTGGGTGTCTGCCATACTT
TAAAAACACTTATTTTTACCTTTTTGTCCTCAGAAAAGGAAATCCTCGC
TTCCATGGCTGCCAAAAGGACAGTGTTTCTGGCTCTCAGCCCTAAGGCC
CTGCAAACTCTAACTTATTTCCCAATTAGTCTGTATCTATACTTGGGCT
CTGTATGTG

In some embodiments, the intronic domain that promotes trans-splicing primarily in the liver comprises or consists of sequences from the gene UBE2V1. In some embodiments, the sequences from gene UBE2V1 comprise or consist of:

(SEQ ID NO: 797)
GTATGCTTGCAGGTTTTGCTGTTTCCTTTCAAATACCATATGTAGTACT
ATTTAATATTTTTCTTGTAATCAACTGACTCTTTTGCTTACATTTATTT
TAAAGGAAACTTTTACCATTATTATAAATGGGAAGTTAGTATATTACTT
ACCCTAAATAAGTTATTCTTAAACATAAATACGTGACTGTAAAACGAAG
TGTTCAATCACATACCACCGAAAGTCCTCTAACATACCACTACCAACTC
TGTCCTTCAGTTGCTTGGACCAAAAACCTGGGAGCTGTAGTTGACTCTT
GTCTCTCACACCTCACAGGCAACCTGATAGCAAATCTCCACAGCTCAGA
CCTCTGAAATGCCTAGAATTACGCCACTCCTCACTACTTTCCTGCTATC
ACCCTGGTCCAAACGGGTATTATCTTTTGCTAGGATTATTGCAGTAGCC
TCCTAGCTGCTTCTGCCCTTGCTCCCTTCACTCTCATCCTCACACAGCA
GTCTGAGGG

In some embodiments, the intronic domain that promotes trans-splicing primarily in the liver comprises or consists of sequences from the gene UBE2V1. In some embodiments, the sequences from gene UBE2V1 comprise or consist of:

(SEQ ID NO: 798)
AACAGCTGTTAACATCTTGTTCTGTATCCTTACCAACTCTATAAAGCTC
CTTAAGATGTCACTTATCATCTATATGAGTTTGCTAGTGCTGTCATAAC
AAAATACCATAGACTGGGTGGGGCTTAAACAACAAATTTCTCACAGCTC
TAGAGACCAGAAGCCCAAGATCAAGGTGTCAGCAGGTTTTGTCACATTC
TGAGGTACTGGGAGTTAGGACTTAAGTATATAAATTTGAGGGGAACACA
GTTCAGCCCAGCCCATAGCACGATCTGTGAACAAGTACCTCAGTCATCA
GCCTTCCTATCAATATGATTCTTTTGATGTTCATTTTTATTAGTGCAGG
CTTGTAAATTAACCAGTAGTAATGTAGTGACTTTTCCCCCGCTAGCCTA
ATTAGGATTATTAAAGATTTAGATTGAACCCTTTCTGAACAATAGTTGT
GTTGTCTGGCTTTTTGCCTATAATGTCACCACTATCTTTCAGTTTTATG
TTTGCTACA

Intronic domains that promote trans-splicing primarily in heart tissue

In some embodiments of the compositions of the disclosure, there may be an intronic domain that promotes trans-splicing primarily in heart tissue.

In some embodiments of the compositions of the disclosure, the intronic domain is derived or isolated from human genes that feature exons that are primarily included in heart tissue during the process of RNA splicing.

In some embodiments, the intronic domain that promotes trans-splicing primarily in the heart comprises or consists of sequences from the gene MASP2. In some embodiments, the sequences from gene MASP2 comprise or consist of:

(SEQ ID NO: 799)
GTGAGTTAAAACAAAGTACACCACGATCAATGACGATTGCTTTGAAATG
TAAACATATTTCTATCTGCAGATATGTGAAAGGAAAGTGAGAAAATCGT
GTGTCAGCATGATTTCTTTTGTCTATCTTTAAATGATAAAAAGCCAAGA
AATAAGTTTTCAGAGGACAAAAAGGATGGCTATGGAAAATGTGATTCTG
AATCCCGCCCTTTTTTCTTTCTTTCTTTATACTTAACTTTAAATTTACA
TAAGCGAGCCCTTTGTTTTAAAGCTAGTTTGGAAAGCTGGTGATTAAAA
TGGGCAACTTGGCCAGGCGCGGTGGCTCACACCTGTATTCCCAGCACTT
TGGGAGGCAGAGGCAGGCAGATCACTTGAGGCCAGGAGTTCAAGACCAG
CCTGGCCAACGTGGTGAAACCCCGTCTCCACTAAAAATACAAAAATTAG
ATGAGGTAGTAGCCCCGTCTCCACTAAAAATACAAAAATTATTAGGTGG
CGGGCGCCT

In some embodiments, the intronic domain that promotes trans-splicing primarily in the heart comprises or consists of sequences from the gene MASP2. In some embodiments, the sequences from gene MASP2 comprise or consist of:

(SEQ ID NO: 800)
TTCACAACAGCCCTGGGGCAACTATTATCGTCAACCGCCCCCCATCTTG
CAGATGAAGAAACTGAGACTCAGAGAGGTTACACATAACCATGGGGAAA
TGGAGAGCTAGGATTTAAACACAGCTCCAGTCCCATGCAAGTCCCAAAG
CCTCCCCTGAGCTCTTTTCCTCGGAGTTACACTCTCTCTTGGTTTTGAG
CCATGGCTCTGAAGTTCCTGGCATGGAGCAGATGTTCTATTTGTGGGGA
CTGCACCATCTTCCCACATAACCCAGCTTTCTCACATACCTTTTGCTCA
GTCCCAGGGAAAACAAGAAACCTTAATTTGCAAAAGCACCTGAGAGTTT
AATCTTGCCTTTGGTTTTAAAGAACCCTACCCCACGTATGGTGAGCTCT
CTTTACATGTTAAGTATTCTTTATAAACTAAGAACTTCAGATGGCTCGA
TTCAGTCTTGGCTGCTGTAGACCACATGAAAACCAACAAGCCTGGTTTC
CTCTGCGCA

In some embodiments, the intronic domain that promotes trans-splicing primarily in the heart comprises or consists of sequences from the gene AGMAT. In some embodiments, the sequences from gene AGMAT comprise or consist of:

(SEQ ID NO: 801)
GTAAGGAGATTCCACCAGTGAAGATCCTGGGGGTGGAGACACTTAACAC
CCATTTTCCTGGGACTTGAAGCGAATCTTTAGGAATCAGGATAGGATTT
TTGTTTGTTTGTTTGAGACGGAGTCTCACTCTGTCGCCCAGGCTGGAGT
GCAGTGGTGCGATCTCGGCTCACTGCAACCTCCGCCTCCCAGGTTCAAG
CGATTCTCCTGCCTCAGCCTCCTGAGTAGCTGAGATTACAGGCACATGC
CACTACGCCCGGCTAATTTTTGTATTTTTAGTAGAGATGGGGTTTCACC
ATGTTGGTCAGGCTGGTCTTGAACTCCTGATCTCGTGATCCACTCACCT
CGGCTTCCCAAAGTGCTGGGATTACGGGTGTCAGCCACCGCGCCTGGCT
GCCAAGGTAGGATTTTAAGAGCAGAGCACCTAGGCCTTTTCTGTTCTCT
GGGCTGAGGATAAGTTTGAATACCCTCTTACTGAAGGGTGATTGATTTT
GGCAAGGGC

In some embodiments, the intronic domain that promotes trans-splicing primarily in the heart comprises or consists of sequences from the gene AGMAT. In some embodiments, the sequences from gene AGMAT comprise or consist of:

(SEQ ID NO: 802)
TGAGATCGCGCCACTGCACTCTAGCCTAGGTGACAAAGTGAGACTCCAT
CTCAAAAGAAAAAAAAAAGAGGGGAGGAAGTCCGGACCCAAGATGAGTT
GCCACCTTCTTAGTTCTGGTCCTCGTACCCTCTAGGCAGAATGGCTCTC
AGATAGACGTGTATGACAAGACCCCAATGGAACAGACCCAAATCCCTTG
CTTTTAGAGCGTGCCAAGGCCAGTCTTCTGGACAGAGCAATTCAAAAGG
TGCTAGCGTGAGCTGCTGTTTCCCACTCTTGCTTTAGCAAAGCTTCTCT
CTGATTCCAACTCCATCAGTCAGCTTTGACCCTGCTTCCTTACCACTCA
TTGTCCCTCCCTTCCTCACCTGTAAAATAGGATAGTTCCCTCTTCATGG
AGATGGAGGGTCAAGTGTCATGTGCACACAGAGCTGAGCATGGGGCTGG
CAAACAGGAAATGCCCTGTAAATGCTGCCAGACAGAAACCTCAGTCCCT
GTCTCTGCA

In some embodiments, the intronic domain that promotes trans-splicing primarily in the heart comprises or consists of sequences from the gene MFSD2A. In some embodiments, the sequences from gene MFSD2A comprise or consist of:

(SEQ ID NO: 803)
GCTTCCGCAATGAATTCCAGAATCTACTCCTGGCCATCATGGTGAGTGG
GACCTGAGCAGGGGCGGGCAGCCTGGGCTGAGGTGACATAGGCTGTGGA
ATGGTTCTTGGAATAGGCAGAGGATGTTTCTCAGGCTGGCCCAAGGTCA
TGAAAGGATGAGGGAGGCTTCTCAGGGTATTTGGTTGGACCTTGCGAAG
GTAGGACTGTGAAAGAAAGAGTTTGAGGTTAGATGTTTGAGTAGTGAGC
ACAGCAAAAGCAAGGGCGAGGACACCAGGAACTTGGCTCAGAGGTTTGA
GAGGGCAGCAGACAAGGTGGGCCTGGACCAGCTCTGAGCTCCCCTGGGG
AGCCGTCAAAGGTGCCTGTCATCTTCGTTGCTGCCCACATGATGTCATC
TGGCTGCTCTTGGGCAGGGCTGGGAGCCACTTTGGGGTTCTGGGAAGGG
CAGGAGGGGCTCTGTGGCTCTAAAGCACCTCCCTTTAACCCCCTTTGTC
CATCCACAG

In some embodiments, the intronic domain that promotes trans-splicing primarily in the heart comprises or consists of sequences from the gene MFSD2A. In some embodiments, the sequences from gene MFSD2A comprise or consist of:

(SEQ ID NO: 804)
TGAGTGGGGTTGAAGAGCAGAGCCTGGGTTGAGTTGGGATGTCTGGTGG
GAACCTCCCAGCTGATTCATCTTCCTGCACCCCCTTCCCTAGTCAGCAG
TGCCATTTCTCATCTTGGTGGCCCTCATGGAGAGTAACCTCATCATTAC
ATATGCGGTAGCTGTGGCAGCTGGCATCAGTGTGGCAGCTGCCTTCTTA
CTACCCTGGTAGGTATATACAGGCCCCCCTCCTCGGGTATCTCCAGCCC
CTAGTCCCCAGTTTTGAAGCTCCTTAGGGAGAGTTCTATGCAGTGTTCT
CCCACAGGCCATTCTGTGGGTCCAGGTTAGGAGTGGGGGAGGTCTGTCC
TGTACAGTTGTACAGGTAGTGAGCTTTGCAAGAACACCTAGTCAGAGTG
AAAAATGGGGGCTGTAATCTGGCCTGGGCTCCACTTGCCACGCTGAGCA
CCTCCAGGCAGGGTTACTTCCTCTTAGAGCAAGAGGCCTTTTCTTATTC
ATGTGAAGG

In some embodiments, the intronic domain that promotes trans-splicing primarily in the heart comprises or consists of sequences from the gene EBNA1BP2. In some embodiments, the sequences from gene EBNA1BP2 comprise or consist of:

(SEQ ID NO: 805)
GAAGCGGAAGTCCCAATCAAAAGTTGAGCAGTAGCTGTGAGTTGCTGGC
GCTGGAGCGATTCGGTGGCACGTGGAGCCACGGCGTGGGAGTAGGGGGC
TGAAGGCAGGCAGCAGCGGCCAGGGCCGCCCTCTGCTAGCCGCTTGGGT
CTCGGGATACCCCGTTTCTTCCTGTAGGTGTGGGACGTGCGTGCGGCGA
GATGGACACTCCCCCGCTCTCGGATTCGGAGTCGGAATCCGATGAATCC
CTTGTCACAGACAGAGAGGTAGGTGTCAGCCGAGCTCGTGGGGCTGGGC
GTGGGAGAGGGGGCCTTACACCCCTGGGAGTGACCGTGATTGTGTCCTG
CAGTTGCAGGATGCGTTTTCCCGAGGGCTTCTGAAGCCAGGCCTCAATG
TCGTGCTAGAGGGGCCGAAGAAGGCCGTGAACGACGTGGTGAGCGTGGG
CACAGGGTCGGGGGTACTCGGACATGGGATGCGCTTCAGGCGCTGTTGG
GCTTGGGAG

In some embodiments, the intronic domain that promotes trans-splicing primarily in the heart comprises or consists of sequences from the gene EBNA1BP2. In some embodiments, the sequences from gene EBNA1BP2 comprise or consist of:

(SEQ ID NO: 806)
AGTTTCATAATACAAGTCGCCATTTATTTCCTCTACATTTATAATACTG
CATATATATTCAAATTGGCCAAAATCTTTCTTTCCAATCATCATGCTGG
GATTATGGGGTTATATGTGGCACATACCATTCCTGTATATGTCAGGATG
GAAAAAGTTTTACCTGGAATGAATTTTTGCCATTTCTGATCCACATTGT
ACTTCACACAGTGATTTCCTGAAGGAAATATAATGATCTGTTCATCGAA
GTAGAAGATATTGTTGGCCACGTGGGATCGAAGACCAAAAACATGCAGC
GTCTGAGCTACCACGGCTGACATGATCTCCCGCCAAACAGTTCTGCTGA
AGACCAAGGCAGCGCTTCAGAGTGGAGAGCACCGCCCCGCTCCGACCCC
GCCCCCGGCCCGGACTCGCCCTCCCCGCCCTTTGCTCCTCCCCTTGTCT
CTCCCCTTTCTCCTCCCCTTGTCCCTCCCCTTTCCCCTCCCCTTGTCCC
TCCCCTTTC

In some embodiments, the intronic domain that promotes trans-splicing primarily in the heart comprises or consists of sequences from the gene PRDX1. In some embodiments, the sequences from gene PRDX1 comprise or consist of:

(SEQ ID NO: 807)
GTGAGTGAGACTCCGTCTCAAAAAAATGTGGAATAGAACAAACTTTAGT
TTTAGGAACTAGACTTTATATTGTAGATCTAGTTGCAATTGACACGTCC
TTGTTTTATGGGAGGCTACATACTTTAAAGGTTTCCAGGGGAGCAAATA
ATTAATTATGATTTTGTGTATGTGTGTTTGTGGGGGGTAGTCATCAGGA
AATGTCTGAGGCCTTTTGCCTTACCGTGTGGGTCTGGCTAATGTGTAAT
GTTTTGACTCAAGATTGTAAAGTCAAATTATTCATCCGGGGAGAGGATG
GTCGTGTAACTGAATCAGCCTCCCAAGATGAACTTTCCCTCTGGCGTAA
GTTTAGGAGTCCTGATATTTTATTTTCCTTTACAACAGAAAGCAGCAAC
CCTGTAATTAAAGAGAACAATGAAGGAAGCTATCTAAGCCATGAGGGTT
ACTGAGGATCTGCGCTGGGGTTAAGGCTGCGAACTTAAGAAATATTTGG
ATAGACAGT

In some embodiments, the intronic domain that promotes trans-splicing primarily in the heart comprises or consists of sequences from the gene PRDX1. In some embodiments, the sequences from gene PRDX1 comprise or consist of:

(SEQ ID NO: 808)
GCCTCCGGGTTCAAGCGATTCCCCTGCCTCAGCCTCCCAAGTAGCTAGG
ACTACAGGCGCGCCACCACACTCGGCTAAGTTTTTGTATTTTAGCAGAG
ACAGGGTTTCACCATGTTGGCCAGGATGGTCTCGATCTCCTGACCTTAT
GATCCGCCCTCCTAGGTGGCCTAAATCTACATTTTTACATAAGATAAGG
TGAATGTTTGAAGAAAAAGGGAATGGAGGAAGAAAACAATTATGTGGTG
GACTCAGGGTAATCTAGTAGTAAGGTACTACTAGAAAGGTAGAAAGTAG
TCTAGTAGAAAGGTAATCTAGTAGTGAACATTTATGGAATTTGTGGAAT
AGAGGCCAGGTGCGGTGGCTCGCGCTTACATTCCTAGCACTTTGGGAGG
CCAAGGCGGGCGGATCACCTGAGGTCAGGCGTTGGAGACCAGCCTAGCC
AACATGATGAAACCTCGTCTCTACTAAAAATACAAAAAATTAGCTGGGC
GTGGTGGTG

In some embodiments, the intronic domain that promotes trans-splicing primarily in the heart comprises or consists of sequences from the gene FGGY. In some embodiments, the sequences from gene FGGY comprise or consist of:

(SEQ ID NO: 809)
CTGCCCAAGACCATGGGAACCCACCTCTTGCATCAGTGTGACTTGGATA
TGAGACCTGGAGTCAAAGGAGGTCATTTTGGAGCTTTAAAATTTGATTG
CCTTGCTGGATTTTGGACTTGCCTGGGCCCTGAAACTCCTTTGTTTTGG
CCAATTTCTCCTGGAATGGCTGTATGTACCTAATACCTGTACCCGCATT
GTATCTAGGAAGTAACTGACTTGTTTTTTATTTTACAGGCTCATGGGTG
GAAGGGACTTGACTTGTCTCAGATGAGACTTTGGACTGTGGACTTTTGG
ATTAATTCTGAAATGAGTAAAGACTTTGGGGGACTTTTGGGAGGGCATG
ATTGGTTTTGAAATGTGAGGACCTGAGATTGGGAGGGGCCAGGGGCGGA
ATGATAATGATTTGGCTGTGTCTCTATTCAAATCTCAACTAACTTATAT
CTCCCAGAATTCCCACATGTTGTGGGAGGGACTTAGGGGGAGGGAATTG
AATCATAAG

In some embodiments, the intronic domain that promotes trans-splicing primarily in the heart comprises or consists of sequences from the gene FGGY. In some embodiments, the sequences from gene FGGY comprise or consist of:

(SEQ ID NO: 810)
TAAGTCAAATTAAACATCCTATTCTTCCCAGTCTCAGGTTTGTCTTTAT
CAGCAGCATAAAAATGGACTAATGCAGTAAGTTAGTAACTTTCCCCCAT
ATTTTTGTTAGTGGAAAGATAGAATAGTTTTTTTTTGTTTTAATGGAAA
GATAGAATAAGAATCTTGGATTCTTTAAGGCCTGAACTTAGATGCCAGC
AGTGGCTGGGAAGGGCACAGGAAGGTGATAGTCCATGATGACGGGTTGA
TACAGACTTACTTAATATATGTTGACTATGTTTTATCCTTATTATGCCT
TCTTATGCCTTCTTATGCCTCTTTCTTTACCCTTCACCTTACAGCCAAT
CCAAGAGTTGGATATCTCAAAAAAATTAGGAGGCCTGGGTGCAAATGTT
TCCTTTCATTTCTACCTTTCCTATACCCCTATTAGGATGTGGAGGTGAA
CAGATGCCTGAAAATCTTGAAAAGGTTGTCCATGGGTCCAACTCTATAT
TAACTAAAT

In some embodiments, the intronic domain that promotes trans-splicing primarily in the heart comprises or consists of sequences from the gene LEPR. In some embodiments, the sequences from gene LEPR comprise or consist of:

(SEQ ID NO: 811)
TCTTTCTCCTTCCCCTTCTCTTCCCTCCCCTCCCATTATATAATCTATG
CTGTTATGTTACCTATTTTATCCACTACCCCATCTTTACTGGAGGTGTT
GTTCTAGTGGACTCTCCAGGAACTCCTCTATCCTCCTACTTCAATCAGG
ACTAGGCTTTCTCTAAAATTACTATAAGCCATCACTCTAAACTTTCCCT
TAACTGGTTTCTGGTTAGTACTGTTTCCTGGATTCCTTTCCTTCATCTT
TCTTAACTACACCCTTAAGTAACCTCCTAAGAAGTATGCGATAAGTCTC
CATCTCTCCATGCCTGAACTGCCTTTTATTCTGCCCTCCTCGATTGTTA
GTTTGGTGGAGTATCAATTCTTAGAAATAGCCTTTCCAAGAGAACATTG
AGATAATTCCTGCCTTAACATATAGTATCCAGTGTTGCTTCTGAGCAGT
CAGATACTATTGTGATTTTTACTCCTCACTGTTTACTTATAATTTCCTT
TTTAAACAG

In some embodiments, the intronic domain that promotes trans-splicing primarily in the heart comprises or consists of sequences from the gene LEPR. In some embodiments, the sequences from gene LEPR comprise or consist of:

(SEQ ID NO: 812)
AAAAAAAAAGAAAGAAAAAAAAATTCTCCTCATTTTTGTTGTTTTGAAT
TTTCAGTGATGAATCTGGAGTGGTTCTTTTTTTATTTAATGTGGTTATT
TTTTGAAGAGACTTTCAATTCCATGTCTCCTGTCAATTCTAAGAAATTA
TATTATTTCTTTGATAATTTCCTCTCCTCTTGTTTCTTTGTTTCTCTTT
CTAGAATCTGTATGTTGGATGATGGACCCTCTGCGTAGATCTTCCACAT
CTCTTACCTTTTATCTCATATTTTCGGTTTCTTTGTTTTGTGTGTGTGT
GTGTGTGTGTTATATTCTGAGACCTTGCCATACCCTCTTCTTCCAGTGC
TTCTGTTGGATTTTTATATACTATTATTTTTGTAAATTTCCAAGTATTC
TTTCTCAGAGTATTCTGTTCTGGTTTTATGCATATTTTCTTAAATGTTG
CTAGAAATACCAATTCGAGGATTTTTGTTCTTTTATTTTAAAAATTCTT
TTTTCTTCT

In some embodiments, the intronic domain that promotes trans-splicing primarily in the heart comprises or consists of sequences from the gene GBP7. In some embodiments, the sequences from gene GBP7 comprise or consist of:

(SEQ ID NO: 813)
AAAGAAAAACTATGAGGTTCTTGCTTCAATTTATCATGGGCTTGAAAGA
ACAGTTTGACAGGGCACTGAGCTGGCAAGGGTTTGAGGATATTGTCAGT
GTCTCATATTTGCTAGTGAGAGTGTGAACTGTTGGAATCATATATGAAA
TATTTAAGTGCACATACCTTTGATCTAGCATTTCCACATTTAGAAATCT
ATCATATAGATTGATTTACTCAAATTGAAAATGGTATATATAAAATAGT
ACTTATGTTGCAGCATTGTTGTAATGCCAAAAGACTGAAAAAAACATTA
AAGCCCCTCTTTCAGGTACTGATAAGGGAAATCTTGGGAAACACTCATA
AAGGAATACTCTACAGCCACTAAAATGAAGGGAGAAGCTCTATTTGAAC
TAACAATGACAAGTCTCCAAGATGTATCATAAGTAAAAAAAAAAAATGA
TGCACGAAGCATGTATAATTACTACCACTGAATTTAGAAAATAATAAAT
GTATGCATA

In some embodiments, the intronic domain that promotes trans-splicing primarily in the heart comprises or consists of sequences from the gene GBP7. In some embodiments, the sequences from gene GBP7 comprise or consist of:

(SEQ ID NO: 814)
GAAAGTATCAGATTTGGCTTGATATGAAATGATACTGAATGATCTCAAA
TTGCTCCATTTGTTTCTGTATCTGCCTGACTCATCCTATACAACTAGAG
TAAAACATATTCCTTGTCCCAAACTGGAGCTTGACAACCAGTGTTTTAT
ACAGGGTCAGCAGTCCCCATTATTTGCTAGATGAGCATGGCCTTGCTAT
ATCGGTGGAAAGGGGAATCAATTAGAATTAAATCTGCATTCCCTCTCAA
AGGGCATTCATTGTGCTGGAACAAAATTCCTCCCCACCTCCAGTGAGAT
GGTGCTGAGGAAATGTCACAAAGGTGTATATCATGGATACTCCCCTTAT
TTAGGTCCAGTCATCTGGAAATGTCAGACCAACTCATCAAAGATTTTCT
AAGATGTTAGAGAAATGGACACATGTTTAACTAATGAAATTGTTAAATA
CAAACCTCGGTCTCTTAAAAATTGGGGATTTTCAAAAGTAGGCTCTTTT
ACTTTTATA

In some embodiments, the intronic domain that promotes trans-splicing primarily in the heart comprises or consists of sequences from the gene ENSA. In some embodiments, the sequences from gene ENSA comprise or consist of:

(SEQ ID NO: 815)
GTGGGTGAAACATCCCTGTGGAGGATCCCAGTTATTGAGTCTTAGTCAC
AGATGATAATGTGATGCTGATTTGCATTTACCTTGCTGGAGGCAAGGCA
GGCGTGGGCAGGCTGTCACAGTCATGGAACATGTGTGGGAGCTGTGAGA
GTCAGAAGGAAAATACATTATCCTCATGAACAGGCCCAGTTTCTATTCC
TGGTCTGGGAGAATAATCATTTTGAGGTTGTTGGCTTGTGGTTGATCTC
CTGGTGGCTAGGATAGCCCCATATTTATCCTTCTCTATATCTTATTCAT
TTCTGTTTCTCCAGTTCCAGGCTATTGCCCTGTACCCAGTAAGGTCCTC
AGTTACCCTTTATGAATAAGTGATTTTGGTGGAACGATCCTCTACTGAG
TGGAGTACAAAGGATGGCAGGGGCTATCCCTTGACTCCAAAACTATATA
CTCTAGCTGGGAAATTGCTTATGTACCTGAAAAAGTCAGAACACACTGG
GGAGAAGAC

In some embodiments, the intronic domain that promotes trans-splicing primarily in the heart comprises or consists of sequences from the gene ENSA. In some embodiments, the sequences from gene ENSA comprise or consist of:

(SEQ ID NO: 816)
GAAGTGAGCACCAGCCATTAATTATACTCCTGCAGCAAATGTATTTTTC
TTGTTCAGAGTGGTCCTCCTTACCAAGAGAGAGTCTACTTTCATAGCCA
TGTAGCTCCTGATCAGATTTTTAGTTCCCAAAGGTACCTAAAGCTGACT
GAGGGATTATATTATCGTTTTTTAAAAAAAAAATTTGGCCAGGCGCAGT
GGCTCACGCCTGTAATCCCAGCACTTTGGGAGGCCGAGGCGGGCGGAGC
ACGAGGTCAGGAGATCGAGACCATAGTGAAACCCCGTCTCTATTAATAA
TACAAACACTTAGCCGGGTGTGGTGGCGGGCGCCTGTAGTCCCAGCTAC
TCGGGAGGCTGAAGCAGGAGAATGGCGTGAACCCGGGAGGCGGAGCTTG
CAGTGAGCCGAGATCGTGCCACTGCACTCCAGCCTGGGCAACATAGCGA
GGCTCCATCTCAAAAAAAAATTTTTTTATTTTCCTTACTCTCCTCTGCA
ATGACTGTA

In some embodiments, the intronic domain that promotes trans-splicing primarily in the heart comprises or consists of sequences from the gene SLC39A1. In some embodiments, the sequences from gene SLC39A1 comprise or consist of:

(SEQ ID NO: 817)
GTGGCCCACGAAGCTACCCAAGACATCTCTGCACAGCCCTAGCCTTTTT
GGCTTCACCCACTCCGTTCGGGAGTTGGGGACCCGGCCTCTACATTCCT
TAGGGGAACTCCAGCTCCAGGTCCTATTTGGAGGCGGCTCCTAGTTTCC
TAGTTTTCTGGGGGCACCAGGTGAAAGGGTGATGAGATATCCATACAGC
CTTTCTCATGCCTCGGTTTCCCCCGTGCAGTCTCCCTGAATAAGTCACT
TGGGAAAGAAAGTGTGAGAAGAGAGGAATGTTCCTGGTACAGGAGTAGA
TGAGTCACCCTGGGTAGGGGAAGGTCACAAAGTGGCTGGGGCCAGGGAG
CTGCACTCTGCTGACCAAGCTTCCCCATTCCTTGCCCCATCTTCCTTTG
CATGATTTTTCCCAACCACTCTGGTCTGTCTTCCTAGGTCTGAGAGTCA
CTGGAGCTACCAGAAGCATCATGGGGCCCTGGGGAGAGCCAGAGCTCCT
GGTGTGGCG

In some embodiments, the intronic domain that promotes trans-splicing primarily in the heart comprises or consists of sequences from the gene SLC39A1. In some embodiments, the sequences from gene SLC39A1 comprise or consist of:

(SEQ ID NO: 818)
AGACTTGACAATGCGGGGTCTCCCTCTTTTAAAAATATCCATATAATCA
AAGCCTTTGTTGACTGGCCCGCTCCGGCCCCTCAGTCCTCCACAGATGC
GCCCCAGGGCCCCAGCCCCGAGGTCCTTTTCCTAATATCCGCTCTTGCT
CTCGCTCACTCTCCTCAGGTCGCCTGCTCCGCCCGCTCCCGCTCCCGAT
CTCTGATTGCTCCTAACTGACGTCACTCCCGGTCTGTCCCCGCCCACTC
GGTGCTGCCATTGGCAGTCGGTCGTGGGTACGCGGCTCCTGTGGGGGAG
GGGGGAGCTATGGGAGGGGGCGGGAATTCCCGACTCTAGGCCGGAAGCG
CGCGGAGACCATGTAGTGAGACCCTCGCGAGGTGAGTGCGGGCTGTCTG
GCAGCGGGGTCCATGTCGGCTGCCCAGGCCTCCCCGGGCCCGGGAACAG
GAGTCCCAGCTCACGCATTCTTCATCTGCTTCCCGCGACGCTTGGCGGC
CTCGCACCC

In some embodiments, the intronic domain that promotes trans-splicing primarily in the heart comprises or consists of sequences from the gene RAB13. In some embodiments, the sequences from gene RAB13 comprise or consist of:

(SEQ ID NO: 819)
GTAAGGCTCCTAAATTCTAGTCCCTCAACCCCTGGCTCTGACCCTTCCC
CCGTGCATGTCTCTGTATAGTAACCAAACCTCTCACCTCTCTGAGGCCT
TCAGGTAACTCTTCAACTGCTGCCAGCTCCTCATTTTCTTGGTACCATC
GCCTTAGTCTCCCCAAATCCACAAGAACTCTTCTCTATTTTTCTCTGTT
CCCCTCTTCCCCTACCCCCACCACCTAGAACTCTCTCGATCCTATCTCC
ATGTTTCCTGTATCCCTGTCCGCCACATCTTTTTCTCCCGATCCGATGC
TAGCCTCTTTCTGTCTTGCTGAGCTTCAAACTCTCCCACTGTGTCCCCA
CCTCACCATCTACTTCCCAGGTTCCCTGCCCCAGTCTCCCTGCACCTCA
GCTGAACAAGGGTTTGGGAAGGGTGGAACAAGGTGGCTGGGTGTGCTGG
AGGGTGGCCCAAGGACTGTGATCATGAATATGCAGCCAGCTTGGTGAGG
AACGTGGAA

In some embodiments, the intronic domain that promotes trans-splicing primarily in the heart comprises or consists of sequences from the gene RAB13. In some embodiments, the sequences from gene RAB13 comprise or consist of:

(SEQ ID NO: 820)
TCGTCGTGGTTAAACCTCTACTTCCCTTCACCCCAGCCTCTCTGGCCTC
AGAATGGAAGAGTAAGGTCCTAAGAGGGGAGCTACAATTACCGAAAGGC
TGTGGAAAATAGCGAGAGAGACAGGCCGGCCTAAGCTAGGAAGGGAAAA
CCGAGAGAACAGAGGCCGGGCTGGCAGCCTTGGCCTGCTCAAAAGGGTG
GGCGGGCAGGGGGTGGAGCCCGGAGGGGTGCCGCCTCTCCTCTTTCCCG
GAGCCTGGGCGGAGAGGGAGGAAAACTTCTTCCTGGCCTGGGCTCCGTG
CCGCTCTGTTTGCCAACCGTCCAGTCCCGCCTACCAGTGCCGGGCGCTC
CCCACCCCTCCCCCGGCTCCCCCGGTGTCCGCCATGGCCAAAGCCTACG
ACCACCTCTTCAAGTTGCTGCTGATCGGGGACTCGGGGGTGGGCAAGAC
TTGTCTGATCATTCGCTTTGCAGAGGACAACTTCAACAACACTTACATC
TCCACCATC

In some embodiments, the intronic domain that promotes trans-splicing primarily in the heart comprises or consists of sequences from the gene FMO4. In some embodiments, the sequences from gene FMO4 comprise or consist of:

(SEQ ID NO: 821)
CTCCCAACGTGCTGGGGTTATAGGCATGAGCCACTATGACCAGCCTAAA
ATGGGGTCATTTTAAGTGTTTACATCTAACCAACTAATAGCTTTAACAC
AAACATGCACACACACACACACACACACACACGTACAATGTGGAGGGTA
TACATACAATATAATATAAATTATATTATTTATACAGCAAAAGCAGATG
AAGAGAGACAGGGCAGGCCAACTGGCACAGGTGCAGGTGTCTGGTGAGT
GTGGCGCAGGCTGACAGCTGGGCCATAGAGGCATCTCTCCTCACAATTC
TGGCAGTAGAGTACCCTGGTTAAGTGAACTGGGCGGGGAATTTTACCTC
CAGAAAATTATTTTCTGTGCTGCATTTCCATTCTGAGCTTTGACTATCA
TGTGCTCTGAAGTTAGAGCAGTGGGAGGCTAGAAATGAGTGGGTGGAGA
ATTTAAATAAAGGAAAAGCCACATGTGACTTTAGTGTTGTTTGTGATTT
TTCCCACAG

In some embodiments, the intronic domain that promotes trans-splicing primarily in the heart comprises or consists of sequences from the gene FMO4. In some embodiments, the sequences from gene FMO4 comprise or consist of:

(SEQ ID NO: 822)
TATGAAATGTAGCTTGCTCTAGGGCAAACTTACAGTATATTCTGTTACA
AAAAAAGGATTCAGAGTATAAAAGCCATTCCTAGAGAAGCAGTATGGCA
GGTTGGAAATAACTCTGCATTAGGAATCAGAAGGATACATTTTTAGTCC
TAACTCTGCCACCAATCAGACGTGGCCTTAGACAAGTCATCTGGGGACA
CCAGGTCTTTTCAGTGGAACACACAGCCTGGCCCCCTACCAGTGGCAAT
CCTGTCTTCACTTAGTCCAGGAAATCTATTCTGACTTCACTCTCTTCCT
ACCTCTCAGGCTGCTCTTTCTCTGTATTTGCTGGTATCGCCTCTTCCTC
ACTCCCAAAATTTAGTGAGCTCCAAACTTAGTCCTCAGGCCTCTTCTCA
TTCCTATTTACACCCACTCCCAGGTGGCCTCATCCAGGCCTGTGGCTTT
AAATATCATCATATACTCAGGGCTCCCAAACTGTGTCTCTGACTCAAAT
CTTTCTCCA

In some embodiments, the intronic domain that promotes trans-splicing primarily in the heart comprises or consists of sequences from the gene CFHR3. In some embodiments, the sequences from gene CFHR3 comprise or consist of:

(SEQ ID NO: 823)
CAGATGACTATTAGAAAGATTATACCTTTCTACAGTTCTGTTTCTACAT
TGTGGGCATAAGAGAAAAATATAAACATAGAATTAAATTAGCAAAATGT
GAGTACATTTGGAGTGCTTGTAGTCACGGCTTGTCGAATGGGGGAAAGG
AGGATAAGTAACCATGACTCCAATGGAACATGTTAGCATAATCCTTTTT
GATATGAAGTCACTAGGGTGGACACAACATATTTTAGGAAAGAAGAGTT
CAGGCTCTAGAGAAAGACTGCCAGGGTTCACGTTTATCACCTCACCTGA
TGATCTCTGCTGTGATGTTGGGGAAGTGCAACTGAACTTATTATATTTT
TGAAATGCTAACGTCAGTATGTAGCACAAGTTAATAACTATTAACTATT
TGGATTATTTTATAATTTTATTTTATCCTAAACTACTCATTAGGATGCA
TTTTATTTGCTCATGAAAGAGAAAATTATGATTGTTAATTGTTTTTTTC
TGCTTTCAG

In some embodiments, the intronic domain that promotes trans-splicing primarily in the heart comprises or consists of sequences from the gene CFHR3. In some embodiments, the sequences from gene CFHR3 comprise or consist of:

(SEQ ID NO: 824)
AAAAAAGAAAAGAGGTTTTGCTAACCCTTTCAGAGCATTGGGAACACAG
CCAGAAGTGCATTAAATGTATATATTAACTTGGGCAATGTTGACACTTT
AGGATGCTGAAGCCAGGTGCAGTGGCACACGCCTGTAATCCTAGCACTT
TGAGAGGCCAAGCTGGCAATCATCAGAGGTCAAAAGTTCAAGACCAGCC
TGTTCAACACGGTGAAACCCTGTCTCTACTAAAAATAGAAAAACTAGCT
CGGCATGATGGCGTGCACCTGTAGTCCCAGCTACTCAGGAGGCTGAGGT
GGGAGAATCACTTTAACCAGTGGGGCAGAGGTTGAACTGAGCCAAGATA
GTGCCACTGCACTCCAGCCTGGGCAATAGAGTGAGACTCTGTCTTAAAA
ATAAATAAATAGGATGCTGAAAATTCCTATTTAAGGAAATAATTCTTTT
TCTGATTTATTTAAGGCTACATTTGTATTTTCTTAATACAAATATTTTG
AAAGTTTCT

In some embodiments, the intronic domain that promotes trans-splicing primarily in the heart comprises or consists of sequences from the gene C4BPB. In some embodiments, the sequences from gene C4BPB comprise or consist of:

(SEQ ID NO: 825)
TGACCTGACAGGAGGTGGAGCTCAGGCAGCAATGTGAGCAAAGCAGAGT
GGCTATAAATACAGCTGAAGCTTTGCTTGCCTGCCCGCTGCTCACTTTC
TGCTGTGTGGCCCAGTTCCTAATAGTCCGTGGACCGGTACCGGTCTGTG
GCCCTGGGGTTGGGGATCCCTGTTATAGAGCAAAGCACATAAGCACAAT
GCGTGAGAATTGGAAAAGTCTCTAAAATTCTTCTAGGCTCCAAAACTAA
ACAAACAACACTGAACAGATCCATTTTCATTGAGATTCTTCTTTTAGTT
CATATTTATTACTGGGATAATATTAATAAGTATATTTAAGCCAGAAAAT
ATAAATGATTTGCCATGTTGAAAACCCAAATCTTTACTTAAGCTAGCCT
GGTTCTGAGCCCCCATTCAGAGAGCTAACTGCTGCTCACTGGCTTCAGA
GTATATAGCTGGGTCTCAGTCCTTATGTTGTAACTTGTGAAAAATTTTC
TTTCTTCAG

In some embodiments, the intronic domain that promotes trans-splicing primarily in the heart comprises or consists of sequences from the gene C4BPB. In some embodiments, the sequences from gene C4BPB comprise or consist of:

(SEQ ID NO: 826)
TTGGTTTCAGATTTACCTCTTATTGGGCTGAAATTGCCAACAGCAGTTG
TATAATGCACCATGTGTGTGTGTGTGTGTGTGTGTGTGTGTGTGTGTGT
GTATGTGTGTGTGTGTGTGTGTGTGTGTTGTTCTCAGAATGTGAAAAGG
CCAAATGTAGTGGTTGTCTAAGGCCAGAATTAGCAACATATTATTGGAG
AATAGAGGGCTTAATTTCCAGTCTGTAAGGAGAATGGAAAACTCTGATC
TCAGCCGAGAGAGTAACTCGTATTTTAACATGAGACCATGAATTATGAA
ACCAAAACTAACCGCAACTTATGAGGCAAAAATTAACCTTCCATTTTGG
AAAAGATGCTTAGAATAGTTACATCCTTAAAAATGACCCACTGGGACAG
AAGGAGAGCTAGGACCCAAATTCTCCTCCCAGTCTTGCCCACATCTGGC
TTACGGAAACAGGTTTCCAAAGCTGCAGGAAAATTGCTTACTGCCTCTT
GAATGAAAC

In some embodiments, the intronic domain that promotes trans-splicing primarily in the heart comprises or consists of sequences from the gene EPHX1. In some embodiments, the sequences from gene EPHX1 comprise or consist of:

(SEQ ID NO: 827)
AAGGCGGCAGCGGGGTTGGAAACCCACCCCCACACAGTCAACACCTGCT
GTCACACCAGGCTAATCAAGAACACTTTTCTTTGGTGCATTAACCACCT
GCTATTAAATAAAGGGCTGTTACTAGGTGGCAAAGTAACACACTGCAGT
AGAGCCTATTTGTAATCTGAGATCAGTGGCTGTAAGGCACCATCCTTGA
GCCAAGCAAGTACTAATATTCTTGTTATCTTAATTAGAATGCAAGAGAT
CGTGTTTTTAAAATTCTCTTTGAATCTGTCACTTTGCCCTCCTCTTCCT
GGGTGATACACCTGGTAGTGCTGGTGGGGGCCATCATAATGCCCCTTGT
CCCAGATTCCCTTCTTTTAGATGGGACTCGAGCACTGATCATTTCAGCC
CTGTATCTCTCAGGTCAGCGTGGTTCAGTTTGCTGTGCAGAGTCCAGGG
GAGATAACCACGCTGTGCACACATGAGATTGGCTGACTTGGCAGGACTG
TGCAATTGT

In some embodiments, the intronic domain that promotes trans-splicing primarily in the heart comprises or consists of sequences from the gene EPHX1. In some embodiments, the sequences from gene EPHX1 comprise or consist of:

(SEQ ID NO: 828)
TAAGGAGGGTGGTTTGTAAAGTTCACTGGGGAGGGTGATCCCTCTTTAT
TGTTCTAATATTATGCTCGCAGCCTCCTTGACAACATCATGCAAAATGT
GTCTTACTAGCTTCTAGTGCATAAAATATTGGTGGAGCTCTTCGCTGTG
CTGGGCCAGTCACCAGTGCTGGGCACTGTGGGTCAAGGCAGTGCCCAGG
ACGGGCACAGCCCTGCCTTCCCAAGCTATGGTTTATTGTGGTAGGATCT
GCCTGCCCAGTGAAGTGTGTTCAGCTGCTCCTGTTGCTGGGGGAAGCTC
TGCTCTCTGCCTTCGGCTGCCTCCCACACGCTCCCATGTGTCTCCCAGT
CCCTAACCCCTAGTCCTGCTTTCAATACCGCAGGGGCTTTTAGCTGACT
TTTCTCCATCTTCTTAAAGGGCAGGATTTCCTGGCAGAGACATAACGTC
TTTCTGGAAATGAAAGAAGCTGATGCATGTTTTTTCTTTTCTGAATTAC
TTAAATAAG

In some embodiments, the intronic domain that promotes trans-splicing primarily in the heart comprises or consists of sequences from the gene RBM34. In some embodiments, the sequences from gene RBM34 comprise or consist of:

(SEQ ID NO: 829)
ATGAAAGCCTGCAATGTACTTTGGAGGGTGGATGGGTGACTTATTTACT
GTGTCAGCAGAATAGACTGAAAAGTTAATACTGATTATCATTGCTGGAA
TCCTAGGCATTCTCTTCCTATGTCCCATCATACACATTAGTATGGGAGG
TGGGTATGATTGCTGCAAACAGTCTTTGTTCTCTGAGCACAGACAGATA
AAGAATTTATGTCAATTTAATATTAAAACTTTATAGAAGTGAGTTGGAT
TTTACCAGCTCAGCACTCTGAACCAGTTATAGCCACATTCTTTGGAGCA
GGAAATAAGTATCATTTATATTTGGTTTTCCCATACATGATTTTTTTTT
TTAACTGCATAGGCATAGTTAGTTGACGTACCATAGTGTACATTTTTGG
GCACTATTACATAATTAATGTATATTAGAATATAGGAAATGTTTATTCT
ATTTTTTTTTTTGAGACAGAGTCTCGCTGTGTCTCACCCAGGCTGGAGT
GCAGTGGTG

In some embodiments, the intronic domain that promotes trans-splicing primarily in the heart comprises or consists of sequences from the gene RBM34. In some embodiments, the sequences from gene RBM34 comprise or consist of:

(SEQ ID NO: 830)
CCTACTTTCCTGAAATGTTCTTAAAAACTGGCAGTAGGCCGGGTGCGGT
GGCTCATGCCTGTAATCCCAGCACTTGAAGAGGCTGATGTGGTTGCATC
ACCTGAGGTCAGGAGTTTGAGACCAGACTGGCCAACATGGCGAAACCCC
GTCTCTACTAAAAATACGAAAATTAGCTGAGCATGGTGGCGGGCGCCTG
TAATCCCAGCTACTTGGGAGGCTGAGGTATGAGAATCACTTGAAGCCGT
GGGGGCGGAGGTTGCAGTGAGCTGAGATCACACCACTGCACTCCAGCCT
GGGCGAAAGTGCAAGACTCTGTCTCAAAAAAAAAAAAAAAAGCCTGGCA
GTAGAGGATTATCAGACAATTTTTCTTAATTCCTTTTTGTAGCACTTAT
TTGATGTTCTGAAGGTCAGCTAATGTGTATTTTTTTTTTTAATTTTAGA
AGCTGAAGTCGTTTTTTAAAGAGTATGGACAAATAGAATCTGTACGATT
TCGTTCTCT

In some embodiments, the intronic domain that promotes trans-splicing primarily in the heart comprises or consists of sequences from the gene KMO. In some embodiments, the sequences from gene KMO comprise or consist of:

(SEQ ID NO: 831)
TCATGAAATTTAAATGTAAGACCTTTTTGTCCAGTATAGATAAGTTCTG
AAATAAGGCAAAAGATAAAGTAAAAAATATATATATAAATATATAATGT
ATGTGCATACATTATATATGTGTGCATATACACACATGTACACATCTAG
ATATGTGCATGTGTGTGTACATATATACCAGGTATAATTATACCAGCTA
TGATTCAAGATTTGAGAACATAAGATGTTCTCTCTGTCTTTTTCCTTAC
ATATACACACACACGCACGTATTCTGAAATCTTGAAATATACCTGGTAT
CAAAAACCTACCTAAGGTTGAGTTTATAAAGACAGAACACAAAGTGTTT
TCTAAGACAACTTCTATTTTATGTAGCAATAGATGTGCGTATCATCGTG
TAGATGCTACTGGTTTTAATTACTGTGGAAACTATTATAAAGCTGAGTC
CGTTTTATAGGTTTGCTATATGAATGCACTAATCAATCATATATTCTTT
TTTCTGCAG

In some embodiments, the intronic domain that promotes trans-splicing primarily in the heart comprises or consists of sequences from the gene KMO. In some embodiments, the sequences from gene KMO comprise or consist of:

(SEQ ID NO: 832)
TAGTATAGAATTTTTTATCAACTGTAATTTTGCATTTGTAATTAA
TGTTGTTTATTACTTACATTTGTACTTAGAATGTATCTACCTAAT
TGATCTCTTCATTGTTTAATCCATTTAGTGATTATAGCTTTGAAA
CTTTGGGATTACTGAGGAAAAATCTGAAGGATTTCCAATTATTTA
CTTTGGAAAGGAGATTTAACTTACATGAATGGATAACAAGAGTTT
ATGTGTGGTGGTGCCTGTCATCTCCACTAAAGACAATTTAGGAGG
AAACAGCCCTTCCAGTAAAGCCAAAGAGATTTAGGTTATCTATCA
GGAAGTGATTCAGCCTTTAAGGTCTGTAGTACATTGGAATACGCA
TTATCAAAAGAGAGCATGGGGATGGGCGCAGCGGCTCACACCTGT
AATACCAGCACTTTGGGAGGCCAAAGCGGGAGGATTGCTTGAGCT
CAGGAGTTCAAGACTGATGTGGCCAACATAGCAAGACCTTGCCTC
TACT

In some embodiments, the intronic domain that promotes trans-splicing primarily in the heart comprises or consists of sequences from the gene RMDN2. In some embodiments, the sequences from gene RMDN2 comprise or consist of:

(SEQ ID NO: 833)
ACTTGTCCTAGGAATATCACCAGTTGTTTTTGTTCTTGAAAAGAA
AAGGATAGAGAAAGAAGACTGAGGAAAATGATTATGTAAAGAAAT
CTGAAAACCATAAAGTATCTCATTGCTTTGTAAGCTTTATGACCA
TGGTAATCAATTTTATTTTAATGGCTAAATGCAAAGGCTATGTTT
CCACCCAAAAGATACTTAAACTGATCAAGTGATTCAAATGTGAAA
CAACCAACCTAAATTCTTAGGGCAGTTTGGTTCATAATATTTGAT
TTATTTAAATATATTCTCTTCACTTGTGGTTACTGTTATTTTAAT
ATTGCCCTGTTATTCTCTCATGTTTTTCTCTCAAATCAGGCTGAA
GAACTATGCCCTGGTTATTCTAATCCCAATTACATGTACTTAGCA
AAGGTAATGAAGACCACTGTTCTGCTTTAAGATCACTTTGAACCT
ATCGCATAAAAACGGATTATGTTTAATATTGGTTATTTCTCATTT
CCAG

In some embodiments, the intronic domain that promotes trans-splicing primarily in the heart comprises or consists of sequences from the gene RMDN2. In some embodiments, the sequences from gene RMDN2 comprise or consist of:

(SEQ ID NO: 834)
TAAGTCCAGAAAGTGACAGTGAGTGCTGTTGTCTTGTTAGTACTA
TTCGAGCTCAAAACAGGAATAACTCGCTTAGATACATTTGTAGTT
TTCTCTATTCAATTTTATGTATTTATTTTTATTCTACCTTTATTG
AAGTATAATTGACAAAAACTATATGTATACAGCATGATGTTTTGA
TAGACATATACATTATGAAATGACTACCACAATCAAGCTAATTAA
CACATTCATCGCCTCACATAGTTACTTTTTTTATTGTTGAGAACA
TTTGAGATCCACTCTCTTAGCAAATTTCAAATATACAATACAGTA
TTATTAACTACAGTCACATTGCTGTCCTTTAAATCTCCAGAACCT
ATTCATCCTGCATAACTGAAACTGTGTACCCTAGACCAATATCTC
CCCATTTCTCCCACTCTTCCAGCCCCTGACAGCCACCCTTCTACT
CTCTCCTTCTATGATGGAGTTTTCTTTCTTTTTTTTTTTATTATA
CTTT

In some embodiments, the intronic domain that promotes trans-splicing primarily in the heart comprises or consists of sequences from the gene SPTBN1. In some embodiments, the sequences from gene SPTBN1 comprise or consist of:

(SEQ ID NO: 835)
GCTCGCCGGGCCGCCGCCGCGTGGTTGGCGCCAGCGCACTGGGGC
AGTCGCGCGCGCCCAGGTGCGGGCCGCGTTACCTCAGCAGACGCT
AGAGAGTGAATGAGCCGCGCGGGCCCGGGACCCTTGGGGCTCTTC
ACTCTCCAGGCCGTCCCGTGGGCGCGCTAGCCTTTTCAAGTGATA
GTAATCGGAGACTTTTCCGTTACATGAGGCTCAACAAAAGATTGT
AATTCCAGCAGCTCTGTTTGGGAAGGCACTACCGCGGCGAGTCCA
GGGCCCGGCCGGGGGTCGGCGGCTGCCGGGCGGCTGGGGCGACCG
CGGACCGTGCGGGACCGGTAGGGGGTCGCGGGCCGGCTAGGCTCC
CCCGCGCCCCTCCTCGTGGTATAGCCTGCATTTCTAATACAATGC
TGTTATGCTAATCAGGTGACATCACCGCCCAGCACACGGCGAGTG
GCTCCTGATAAAATTACAAACCGGCGGCCGCCGCGGGCAGCTGGG
AGGA

In some embodiments, the intronic domain that promotes trans-splicing primarily in the heart comprises or consists of sequences from the gene SPTBN1. In some embodiments, the sequences from gene SPTBN1 comprise or consist of:

(SEQ ID NO: 836)
TAAGCCCCCTCCCAAAGGCCGGGCCTGTCCTGGGTGCCAACGGGG
GTTCTTGGAGGCTTTATTTGTGACCCTAATGAAGACGGGCGGCTT
CACAGCTCGGCCCCAGACCCTGTGGTTGGCTGCGGGCACCCCATT
CACGACTTAGGGAAGGCAGTTGCTCATACTCCACTCCAGAAGAGC
TACCATTTGGAGTCCCTGTCTCTCCAGCACCTTTTTGGCTTTCTA
GGTTGTCAGATAACTGCTGCACACACATTTTCTTTACTTTTAGCT
AACTTTACCTCCTAGTAGATCAGTGCTCATAGACTTATTAGTATG
CAGGTATTTGATTCCGGGTCGCCTCTGGCTCCCAGCCCCCAGCCT
CTTACCTCTGGGAAGCTTTAAAGGTGTAAAGTGCCGTGCTCAGAC
CCAGTCCTGTAGAACAATAGCAAAGGGGAGGAGGGAGGGGTCTTT
TTGTTCCTAAGCCTTTGGTAAATTACAGTTTTTTATTCAGAAGCT
TAAT

In some embodiments, the intronic domain that promotes trans-splicing primarily in the heart comprises or consists of sequences from the gene NFU1. In some embodiments, the sequences from gene NFU1 comprise or consist of:

(SEQ ID NO: 837)
GTGGGTAGTGCATCTGATGGAGCCGGCGGGTCCGCCCTGTCGCCG
TAGTTGGGGGTGAGCCCTGAGGCTGTCTGTGCGGGAGCTCGGCGC
TTCTGTGGCCTAATCCGGGCGGACTCTCTGGAGCTTCCTCTCAGG
CTCAGAGACCCAGTTCTTGAGTCTCTGCGGTACGGAAGCTTGTAG
AGACTGGCTGTAGAGCCAGAACGGAGGAGCTGTTTTAAAATCGTG
TGCCTCTCCCGCCCCCAGGCCCTCTACCGGGTGCATTCCCTCCTC
TTCTAAACGCAGAATCAGAATGTCACACAGAAGGGAAGAAAATAC
CTTGTCTCGTTTCCTCATTTTCCCAGTAAGGCCCTGAAAGAGGAG
GTGACATGTCCAAGGTTACACAGTTTCTAAGTGATGGAATTCCGA
CGAGAACCAGGACCCCTAGATTCCCTGCACCCACCACCATTAAGC
CCACTCTGTTCCCTTGTTTTTTCCGCCTCCCTGAGCCCTACCTTT
GAGT

In some embodiments, the intronic domain that promotes trans-splicing primarily in the heart comprises or consists of sequences from the gene NFU1. In some embodiments, the sequences from gene NFU1 comprise or consist of:

(SEQ ID NO: 838)
CGAGAACCTTTTAGAAGGAATGCTCCAGGGAGTCTTTGGGGGCAG
TCACTGAGCTGGGCCTGGAGGCTGAGTAGGGTCCTAGAGGAGGGA
ATGGGTCAGGTTTAGGGGCTGGAAACAGCATTCGCAAGGCAGCAC
CAGCACCAGCTAAAAACCTAGCCAACAGGGAAAACAAAGTACGGA
AACACGCATGCGTTCACAGTGCTGCGTGAATTAACCAAGCGTTGG
CGCAAACGGGAAAGGTTCCCCGGCCTCTCTTGGTCAGGGTGACGC
AGTAGCCTGCAAACCTCGGCGCGTAGGCCACCGCACTTATCCGCA
GCAGGACCGCCCGCAGCCGGTAGGGTGGGCTCTTCCCAGTGCCCG
CCCAGCTACCGGCCAGCCTGCGGCTGCGCAGATCTTTCGTGGTTC
TGTCAGGGAGACCCTTAGGCACTCCGGACTAAGATGGCGGCGACG
GCCAGGCGGGGCTGGGGAGCTGCGGCTGTTGCCGCCGGGCTGCGC
AGGC

In some embodiments, the intronic domain that promotes trans-splicing primarily in the heart comprises or consists of sequences from the gene PCBP1-AS1. In some embodiments, the sequences from gene PCBP1-AS1 comprise or consist of:

(SEQ ID NO: 839)
GTTGGTTGAGGGGTTTAACAAGGCTAAGAAGGTGAGTCAGGTTTA
GACTTTGGAAGTTGAACCACAGGATTTATGCTACATACAGAGGAA
TGATTGTTGCTTCCTCTTATGTTAAGTTGGTATGTCACGTAAAAC
CTTTCAGAGTGCTTTCACGTTACTTGTTTAATCATCACAGACAAC
TTTGCCAGTAGGGTCTTATGCCCATTTTACAGAAGAGCCATTTTT
GGATTAGAGGAGTGAAGTGACTTGCCCAACGTTACACAGCCTGTT
AGAATTTGAGCTGAACCTCTTGACTCTTAGTTATGGATTACACCT
TTCCATGGCTTCTGATGCTTTTGATATGGACTTTGATGTGTGGGA
TAGCTTTACCTTAAACTCAAAAGGCTTTCTAGCCAGTTACTATTT
TTCTGTCCCCAAACAGCCAATGCATTTACCTTAAGTTCCTAACTC
TTGTTTTAGAAAATCTCTTCCCTCTTTTCCCTGATGTTTACATTT
ATGG

In some embodiments, the intronic domain that promotes trans-splicing primarily in the heart comprises or consists of sequences from the gene PCBP1-AS1. In some embodiments, the sequences from gene PCBP1-AS1 comprise or consist of:

(SEQ ID NO: 840)
CTACCTACTGAAATATTTCCATAATTTCCTGTTATGTACTCCTGG
GACTTAGTGCACCTCCTCAGTGAAGCCTTCCCTTTCCCAGCTTCC
CCAGCTCTTAGCCTCCCTCCTTCCCCGAGAGTCTTAACAGTTTCT
CCCTTCGGTAGCCCACGTTTGGACGTTGGTTAAATTGTAATGTGT
CTTGGGGTTGGTAACATGTCATACAGACACCTTGGGTGATGCTGT
GGTCTGGCAAAGAGGTCATTGGACCTTGGAATCTGAGGCTCTGGG
CCCTAGTCCTAGCTCTGCCCCTGTCTAGCTCTGAGACTTCTACAA
CTCATCTGTCAGGGTTTCAGTTCTCCCTTTTATAAAGCAAGAGAT
TAAATGATCTCTAAGGGTCCTTCTACTTATGTCCAGGGACTGATG
ATATTTGATGACTTTGGTTATTTTTCTTTGGCGGGGTGGGAGGTT
TATGTAAATGTTTTCTGCACTTTTGGTAAAATCTCCACTCCCTTC
CTCA

In some embodiments, the intronic domain that promotes trans-splicing primarily in the heart comprises or consists of sequences from the gene IL1R1. In some embodiments, the sequences from gene IL1R1 comprise or consist of:

(SEQ ID NO: 841)
TCCTTAAGTGAAAAATCTGTGAGGAGTTTTCTATCTTTCTAAGTC
TTTGTGGCACACTTATCACATGCATAGTACTATTTCTCAAGTTAC
CCAGGCACACACAGATGTATGGCTTTCAGGAAGCCATACATCTGT
GTAGCTCTGAGGCCCAGCCTTGTTCAACACTTCACTCAGAAGAAT
TGCTGGCCTGATGACCAACAATAATTGTCAACAACTTAAAACTTC
ATTGACGGCTCCTAAAAAAAATCAGTGTGCATGTGTGTGTGTATG
TGTGTGTGCGTGTGTGTGTGTGTGTGTGTATGAGTGTGTGTATAA
CATTCATTACTGCAAACTGGAATGGAGTGGGGCTGGAAGGCAGCC
CAGAAGCAAGGCTCAGAGCCTGGAGCTGCTGACCTCTTGGGGACA
CTGCAGCCCTGGCCTGGCCTACTTTTCTCTTCTCCCATATCTGGA
AAATGAAAGTAGCCTGACGTATCCGGGGACCCGGCCCAAGACTCC
ACCC

In some embodiments, the intronic domain that promotes trans-splicing primarily in the heart comprises or consists of sequences from the gene IL1R1. In some embodiments, the sequences from gene IL1R1 comprise or consist of:

(SEQ ID NO: 842)
TAAGGTTTTAAAGGGCCCGCCCTGGCTTCCTTGGGCCTGTAGTTT
GTGACTGTGTCGTTTTCAGAGTGGGAAGGCCCAGAGTTGGCCCCA
TGGAATTCTTTCTGCTTCTAGGGTGTTTGAAGAGATAGAAAGCCA
GGGTAAGGTTCACCGAACCTCCAAGCAAAAATAAATTTCCAGAAG
GGGCCCCTTTACTCTGAGGACATTGAATAGCAACAAAAACTACCA
CTGAAGAGTCATTTTGTCCCAGGCCCTGTGCTGGAAACTTTGAAC
ACCTGTGTTCACCCCTCCCTCATGACACTCATGAAGATATACCAT
GGCTGTCTTTATTTTCTAGGTGGGAATCTCAGGCTTGAGGGTTGG
GGTGGCTTGGGGAAGGTGTGTATCTAAGAGGTAGCTCTTATTTAT
TTTTACTTCTAGAGATTAAGACACATTCTTTTTCAAGATAGACAT
GGAACTGTCAAAAAGGATTAAAAGTTAAATAAGGATAAGGTTATG
TAAA

In some embodiments, the intronic domain that promotes trans-splicing primarily in the heart comprises or consists of sequences from the gene DBI. In some embodiments, the sequences from gene DBI comprise or consist of:

(SEQ ID NO: 843)
AGCGCGTGCACAGCCAGGCTGCGAAGGTGCAGCGGGCGGGAGGCC
CGTTGGGGGCTCAGCCGGCTGCCAGAAGCTCTCGGGCTCTTTCCT
TCCGTGCCCCTCACTTGCTCATGGGCCCATGCCTAGCCCTGATTC
GTTGGACAGAGCCTTGTGAGCGGGATTTTCCGTTTGGGGATTTCT
AAATCTGCTGCCCACCCCGCAACTGCCGGAAAGTTGCCCATGGGG
TGGACTTCGCTGTGTAGCGGGAGAGGGGTGGGAGTCGAGGGTGCT
TGATGGAGAGATGGGGGAAGGGGTTGCACGGATTGGAGGAGCGAG
GAGACTCAGTCCCCATCCCGAAGCACAGGGCAGGACGTCGCGGCG
GAGTGGGGAAGCGAGGAGTCCGTGGCCGAGAGCTTGGAGGTCAGG
GGAAGTACGGGGCCGGCTGCTCAGAGTGCGGGACGAGGAGAATCG
CGGCCCGGGGAGAGGTGACCCAGGGGCCCCTCCCTTCTCTCCAGT
GTAG

In some embodiments, the intronic domain that promotes trans-splicing primarily in the heart comprises or consists of sequences from the gene DBI. In some embodiments, the sequences from gene DBI comprise or consist of:

(SEQ ID NO: 844)
TCGGTGTTGAACGCGCGGGCCCCAGGGGGAGGGAGGGGACCAACG
GGCTCCGGCGCTGACACCGCGGCACTCATGCCCTGTCCCCTTTCA
GCTGTTTCCAGCATACTGTGCCCCGTCTGTCCTCAGGCCAGGGCT
TCGCTGCAGCCCCGGCCACTCCCTAGTGCCTGGCCCGGTGGTGGC
CAGGCAGTTGGCCGCGCTGCTTCTCCCGCAGAGGGGACCCCCACT
GGGGGCGAAGGCTTGGCCTGCCCTCTTCACTGCTGTATTTCCAGA
CCTGATGCCTGCGTTTGTGAGAGCTCTGGATATATGGTTTTCGAT
TGAATGAGTGAACTGGAGGGGCTTCCCCTTCTTGTGTTGCTGAAT
CTTTCTAGCTGCCCTGTTGGGGCAGGGAGGGGCAGACACACTTCA
GGGGCTGCATTGCCCGAAGGGTGCCACCTTTCCCACCTCTCCATC
CCCGTAACTGGGCTGTCATCAGGCCACAGTAGGATTCTTACCCTC
TCCC

In some embodiments, the intronic domain that promotes trans-splicing primarily in the heart comprises or consists of sequences from the gene HNMT. In some embodiments, the sequences from gene HNMT comprise or consist of:

(SEQ ID NO: 845)
TCCCTAGAGTTGACTATTGCCTTGATTTTGGTTTCTTTTCTTAGT
TCTCCATGTACCTATTACTATTTTGATCCCCAAGCAGATATATTA
ATCTCCTCTCAATATATGCAAACATATCAAGGAATCTGTTTATTT
TACCCCAAGGGAACAACTTTTCTGAAGCCCTCTGTTCTTTTTTTC
AGTTTAGGAAGATCTGCTTTGGCCTGCTATACAACTATTAAGGAG
GGAATTTATTTCACTTTCTGAGAATCATTTTCTTTCTCCATTATT
TGATTCCCTGATTCTATATCTCATGCCTTTCTTTTCTTTGTTTCC
TCGGTCCTTTGTTTCCTGAACAATTCTTTAAGAACTTTAAGAGTG
AACGAGAGGTAAACTTTTTGAGACTTTGTATGTTTAAAAATCCCT
TGGTTCTATGTTAATCGTTTGACTGGATATAGAACTCTATACTGA
AAATGGTTTTGTATAATAGATCTAAAGGTACTTTTCCACTGTCTT
TTAG

In some embodiments, the intronic domain that promotes trans-splicing primarily in the heart comprises or consists of sequences from the gene HNMT. In some embodiments, the sequences from gene HNMT comprise or consist of:

(SEQ ID NO: 846)
CCTTGGCATGTGGAATAACTTTTACATTGCTATGTGTTTTGTTTT
CCCTATTTTGAACTTTTTAGAATCTTCTTACGTCTAGTATTCTGA
AATTTTAAAATACTGAGCTGTGAAATGGTTCCTTTTTACCCATTT
TATGGGTACTCCCTGGGCTATTTCAATTGAGATTCAGATCCTTCA
GTTATGCGGAACTTTGGTAATCATTCTTCACCACTGTTTTCTTTT
TCTGGAACTTTTATTCATCAAATACTTCCTGGATTGATTACCTAA
TTTTCTTATATTTCCAGTTTGCTACAACTTTCCTCTTTCAGCCCA
TGTATTAACTTTTTACTATTATATTTTTAATTTCCAAGAATGTTT
TCTATAGCATTCTTTTCATCATTCCCAGATGTAATACCTTCTTAT
TCATTACAAAATGTTCCTCTGCTCTCTCTGCCTCTTTCCATCTCT
GTTTCTTATGTTAGACAGCTCCCCAACCACCTAAAGAGCCTTGGT
TTTC

In some embodiments, the intronic domain that promotes trans-splicing primarily in the heart comprises or consists of sequences from the gene KYNU. In some embodiments, the sequences from gene KYNU comprise or consist of:

(SEQ ID NO: 847)
GATCTGCCCACCTTGGCATCCCAAAGTGCCAGGATTACAGGCGTG
AGCCACACACCCGGCATGTTTTCTTTTTTTGGAAACAATAACAGG
ATATCAGATTGATAAATGCAATACCAATTTCTGATAATTTATCTT
GCTATATAATACTTCATGGACTTCTTGCACTTTGTGGTGTTTTAT
TAGTCAGGAATAAGGAGAAAACAACTTATAGCTGTGGAAAGAGTA
CAAATTAATTTTTAAAAACTGGAGAATAAATCAAGCAATTCGGCT
TTTGAAGACTTTCACTTGAAAAAACACTTTTTACTTTTTAATTAT
CATTTCTTAATAATAAAAATTATGCTCAAATGCTGCTCTTATGCC
AGATTTTTAAAGAACCTTAATCTGAAAAAAAAAACTATTTTATAA
TGCAAAAGGGCTCACGGTGAAATTTAGATCGGCAATATGAAATTA
CAAATTTATTATTATCGATATGACCTAACTTGTGCCTAACTTGAT
TTAG

In some embodiments, the intronic domain that promotes trans-splicing primarily in the heart comprises or consists of sequences from the gene KYNU. In some embodiments, the sequences from gene KYNU comprise or consist of:

(SEQ ID NO: 848)
TATGCACGCCATTTACTTCTTCCCACCTTACTCCAAACATCACTC
TACTTAAGAGTGTTCTAATTGCATGACTTGTGAAGTACTTTAGCT
TGTGTCTGAGTAAAACTATTTCAAAAGTTAAAAAAAAAAAGAGTA
AACCTTGGGCCAGGCACGGTGGCTCACGCCTGTAATCCCAGCACT
TGGGAGGCTGGAGCAGGGGGATCACGAGGTCAGGAGTTTGAGACC
AGCCTGACAAAAATGGCGAAACCCCATCTCTACTGAAAATACAAA
AATTAGCTGGGCATGGTGGTGGGCACCTGTAATCCCAGCTATTCG
GGAGGCTGAGGCAGAAGAGTCACTTGAACTCGGGCGGTGGAGATT
GCAGTGAGCCAAGATCGTGCCCCTGCACTCCAGCCTGGGCAACAG
AGCAAGACTCCATCTCAAAAAAAAAAAAGCGAGTAAATCTTACAT
TTTTAAAAGAAAATCTGTAATCTTTTTTGCTTCAGGCATCTCTAT
TTAA

In some embodiments, the intronic domain that promotes trans-splicing primarily in the heart comprises or consists of sequences from the gene GRB14. In some embodiments, the sequences from gene GRB14 comprise or consist of:

(SEQ ID NO: 849)
GTAAGTTTGGTATTTTTAATTTTGAGTACTTGTGAATTGGATTTC
TCCTATAAGCAGAAATTATTTATTCCATAGCCACAAAGAGTTCTT
GATTTGGGCTTATCATAAAACAGAAGTGACATTGTTCTATAAATG
AGGTCATCACTGTATTCTAACAGTATCTATATTATACCTGGTACA
TAGTGTGTGTCTGGTATTTGTATATAAATGGACAAATAACGGAAT
AGAGTATCTTGTTCTTAACCAGTGATCACTAATATTACCCATTAT
GTCCTGATTCTAGCATCTCAGATATCAATTGATGGGGGAAAATGA
ACTTCTTTTTAAGTGTCAATATTTTGATTTGAACATATGATTTCG
AAGTATGGTTTATATTATGACATTATGTCAAATGTATTTTTTTAG
GTATGATGCAACATGTATTAAGTAAAAAGCTGTTTCTTAAGTGTT
TTGAACACTTATTTTTTTGTGTGTTTACATCCCTATTGTCCTTTC
TCTA

In some embodiments, the intronic domain that promotes trans-splicing primarily in the heart comprises or consists of sequences from the gene GRB14. In some embodiments, the sequences from gene GRB14 comprise or consist of:

(SEQ ID NO: 850)
AAAAACACTGAGAGAAAAACACTGAAATTGAAAAACACTTTCAAT
TTATTTGAAAGATAACTTTCAAAGAAATTTTCATAACTCAAATGT
GTTTCTTTTTTCCAGAGAAGTATATCAGAGAATTCCCTGGTAGCA
ATGGACTTCTCAGGCCAGAAAAGCAGAGTTATAGAAAATCCCACT
GAAGCCCTTTCAGTTGCGGTTGAAGAAGGACTCGCTTGGAGGGTA
CAAGTAGCTTCACATGCTTCAAAATATTTATGATTTCAGTTTGTT
TCACCTAAAAACTTGAGATTTGTTTTCTCCTTCCTTGCTCAGAAA
AAAGGATGTTTACGCCTGGGCACTCACGGTAGCCCCACTGCCTCT
TCACAGAGCTCTGCCACAAACATGGGTATGTCTGTTCATAGTCAT
TGCCACTGAGTAGACGGATAGACATGGAAAGGACATGTAATGAAA
TTGCAGTCATCTCAAACAAAGCTTTGCATTCATCCATCCTATTTC
TTTA

In some embodiments, the intronic domain that promotes trans-splicing primarily in the heart comprises or consists of sequences from the gene SPATS2L. In some embodiments, the sequences from gene SPATS2L comprise or consist of:

(SEQ ID NO: 851)
TCAGAAACAGTTATTGGTATTGCCAAAAAAAAAAAAAAAAAAGGA
AGCTAAGAAGTCACCCTTCTAAATAAATAAATAAATAAATAAATA
AATAAAAACCTGAATTGGAGAGCTGCCTGCATTTCACTCTACTGC
CTCTGGAAGAAGAGTGTTATCTAAAGTGCTTCCATCATGAATAAT
GATTGAGAAACTGCTTTTACTCTCCCGACTACTGTACTCCCAGGC
AGAAACCCACGGCTGAAGCCCACGATGCCCATGTGTAACTTGGTT
ATTACACACACGTTTGGAGCCCAGTGTGCAGACCCGCAGCGCAGA
GGAGGCGGCGGAACACCCCGGGTTGGTCGGGTTTCCAAGGGCTGA
CCCGAGCTCCAGCACTTTTTCCGCGCCTGATTTTTCAGGTCATTT
TCGAATGGGCTATCTTTGCTTACATTTTGAGCTTGGATCCTCTCC
AGAAAGCATTACTACCTTCTAAAAGGAAAAACATCTCGTATTTGC
AGAC

In some embodiments, the intronic domain that promotes trans-splicing primarily in the heart comprises or consists of sequences from the gene SPATS2L. In some embodiments, the sequences from gene SPATS2L comprise or consist of:

(SEQ ID NO: 852)
TAGGAGAAGATGATTCTCTTGCAACACGTGCGGATTGTGACAAAA
TCTTTCATTAACAAGGGGAGTTTCGGTGAAGTGGAGGTTTGGGGA
AAGGCGAGGAAGTCGGTCTGGAGCAAGCAAGCAAAGTGCGGAAGC
TGTACTGGGATTCTTCTAGAAAGTGGGGTGGGAAAGGAGCTAGGG
AGGGCGTGTGGAGGGACGAGATCTGTGTCAGAACGTGCGTGTGAG
CGGATACAAAACCCGAGAGAGGCGTGAGCAGCGCTGTGTTTGCGA
GCGGGAGCGAGGGGCGCCGGCTGGGGTGTGTGCTCCTGGTGAGTG
ACACCGAGGGGAAGGCGGGCGGGTCGGCGGGCGGGGGCAGCCTGC
GAGGGGCGGGAGTGTCCCTGCGTGGGGCGGCCGAGCCGGAGTTGT
GTCAGTGAAGGAATCCAGTCCGGGGGCCGAGCTGGCTGCGCCCTC
CGCTGCAAGCGCCGGCAGCGCGGGGCGAGCTCCGGACGGCGCGCG
GCCC

In some embodiments, the intronic domain that promotes trans-splicing primarily in the heart comprises or consists of sequences from the gene CPS1. In some embodiments, the sequences from gene CPS1 comprise or consist of:

(SEQ ID NO: 853)
AGGGCTGAGGATCGCTCCATCTTCTCAGCTGTCTTGGATGAGCTG
AAGGTGGCTCAGGCACCTTGGAAAGCTGTTAATACTTTGGTAAGG
AGAGAAACAAGTATCTGTTTCTAATGTTCTATTTTGAAGAGCTGC
AACCTGAATGTTGACTGAATGTTGACTATTGGATATGTTAACTAC
AGGGCAGATAGAATGCATACACTTAGTGAATTTAAAGTTGTTGTA
TATCCATGATTGCAGTTAATGCTGATTATGAGTTGATAGCAGTAT
TCTCATTAAGATTCAGACAATGAAAATAATTTCAAACCAGTTTTG
AACAAGATTTGATATTTAGAATCAGAAGCTAGGAATTTTATGTAT
TTCAGTGTCCAATTTACATTTAATGGTGCCTTTAGAGAATAAAGA
AGCTGGGCTACCACTCGAGCTAATGATACATTCTGTAATTTTATT
GCATATTTTAAACTACTCATACATTTTTATTGTTGTTTCTATTAA
TTAG

In some embodiments, the intronic domain that promotes trans-splicing primarily in the heart comprises or consists of sequences from the gene CPS1. In some embodiments, the sequences from gene CPS1 comprise or consist of:

(SEQ ID NO: 854)
TAACACTGTATATTGTTTTCCAAAACAATGATTCAAAAATTGGGA
GATACAGAAATGCATGCTGTATGCTAATAGCACTAGGGTTCTATA
TGTAGTAATTTATAAATCCATTAACAATTTCCAAACCATCACAGC
CAGTTTAAGGACCAATGTGTGGTCTTCTAGTTTAATGCTTTCCCT
TCAGGGAGTGGCCATAGGAAGACTACAGTTCTTCATCTAGAAGTG
TAGTGTAAGGCATCTCCTTACCAAACAGAGAGGGTAGCTTTATAT
TAAAGAACAGGACAATTTAAAAAAGACACACAAATTGAGGTATCT
CTCGCCAGCGTGTTTTCTGATACCCAGAAATGACAGCTTATCTTT
GACTATTGTCAGCACCTTCTGATAGTGCCTTTGTTTTAAAATTTT
CAGTTTTTGAGATCCGATTGTTTAATAGATAAATTATAAGGGTGA
GGAACAGGAACAGTGAGGATCTTTTATATTAAACTTTAAAACAAA
GTTT

In some embodiments, the intronic domain that promotes trans-splicing primarily in the heart comprises or consists of sequences from the gene PECR. In some embodiments, the sequences from gene PECR comprise or consist of:

(SEQ ID NO: 855)
GTAGGTAAACGATCTATATTGAAATTTATTGACAAATTATGCTTT
CTGATTCCATTTGTGATGTTGATAGAGGTATTTGGTAATAAGCAA
GTATAGCATAAAAGATTGACTTTAAAACATAGATAAAATGGAAAG
CCTAATTTTCTTTTTTTTTTTTTTTTTTTTTTGAGATGGAGTCTC
TGTCGCCCAGGCCGGAGTGCAGTGGCATGATCGTGGCTCACTATA
ACTTCCACCTCCAAGGCTCAAGTGATTCTTGTGCCTCAGCCTCCT
AAGTAGCTGGGACTACAGGCACAAGCCACCATGCCCAGCTAATTT
TTTGGATTTTAGTAGAGATGGGTTTTCACCATATTGCCCTGGGTG
GTCTCGAACTCCTGAGCTCAGGAGATCTGTTCGCCATGGCCTCTC
AAAGTGCTGGGATTACAGATGTGAGCCACCGCACCCGGCCCAGAA
GGTCTAATTATCATTATGGAAGTACTCTTTGATTTACAGCCAGAT
TGTC

In some embodiments, the intronic domain that promotes trans-splicing primarily in the heart comprises or consists of sequences from the gene PECR. In some embodiments, the sequences from gene PECR comprise or consist of:

(SEQ ID NO: 856)
CCCGGTAATTTTTGTATTTTTAGTAGAGATGGGGTTTCACCATGT
TGGCCAGGCTGGTCTTGAACTCTTGACCTCAGGTGATCCGCCCGC
CTTGGCCTCCCAAAGTGCTAGGATTATAGGTGTGAGCCACTGCAC
CTGGCCTCCTTTGTTATTTAAAATCGATGTTAAAAATAACCACAG
GCTTGAAATCCTTAAAATTACCAATGTGTTTTCCTTTGTAATCAA
AGTTCTTTAAAGTATTCCTCTTACGTAGTGATTTGGGGAGCCTTA
TCTGGGTCTATGCCATGTGAGATTTGCTTGATCATCAGCTGTGGC
TTGGATTTAATATTGAAGGACATAGTTTTTTCTTCGTTGTTTAGA
CAGAAGCTCAACAAGACAGACTCCATAGCAAGAAGTCTCTTGTGG
AATTCTGTCAGGTAGTTGGCACACTTGTGAGCTTGATGAACAAGA
GAAATATTCAACAGAAGCTGATTGTCATGTTTATGTTTTGTCTTT
CAAA

In some embodiments, the intronic domain that promotes trans-splicing primarily in the heart comprises or consists of sequences from the gene SP100. In some embodiments, the sequences from gene SP100 comprise or consist of:

(SEQ ID NO: 857)
GTAGTTATTTTCAAATCTTTTCGAAAGGAAAGAAGGAAGGAAGGA
AAGAAAGACGCAGGAATATGTATAAAGGTATAGCAAACATCACAC
TTAATGGTGCTGCTAAGTGCTTCTTCCTTGAAATTGGAAATAAGG
CAAGGAAGTCCACTCTCATCACTTCTCTTCAACATTGTATTCAAA
CTCCTAACCAGTGAAATGAGACCAAAAAATAGAAATAATTGTTTG
GAAAGAAACAGTAAGTCAGTCTATTTGTTCATGATATAATTATTT
TCTTTAAAATACAAAGGAAATCTATAAATAATTTACTAGAAATAC
TACATGAATTTAGCAATGCCATAAATTCATGGTTAATATGTTTCA
AACCAATTATATTTTCATATAATAGTAATAAGGAATTTGAAAAGC
ATTATCATTTAAAGTAACACCAAAACAATAAAATAACTAGGAATA
AATTTATTATTTTTTAAATTTAATTAAATTAAATTTTTTATTTCA
ATAG

In some embodiments, the intronic domain that promotes trans-splicing primarily in the heart comprises or consists of sequences from the gene SP100. In some embodiments, the sequences from gene SP100 comprise or consist of:

(SEQ ID NO: 858)
TAAGTAAATGGAGAGATATACAATGTTAACAGATTGGAAGCTCAA
TGTTGTTGTCATCTCCCCCAAATTTATCTGTAGCTTCAATTCAAA
TCCAAAAAAAAGTCCTAAAGTTTTTTCTTGAAGAAATTGACAAAC
TGATTTGAAAAGATAATGTATAAAAGCAAAGAATCTAGAAAATCC
AAGCCAATCTTTTCTAATTGACTTCAAGACTTTTGTTAAGCCATG
ATATTTAAGGCAAAATCTTACCAGCATAAGGGTAGACAAATAGAT
CAATGGTATAGCATAGAAAGTACATATATAAACCCATGCTAATAT
TGTAAATTGTTTTTTGAATATTTTCATGACCTTGGAGTAGGCAAA
TATTTCTTAGATAGTACACTAAAGTCATTAATTATAAAAGAAAAA
AAAATAAATTGGATTTCATCAAAATTACAGATTTCTTCCCATCAA
AAGACATGATTAAGAAAATGAAAGAGAAGCTACAGACTGGGTGAA
AATA

In some embodiments, the intronic domain that promotes trans-splicing primarily in the heart comprises or consists of sequences from the gene TRPM8. In some embodiments, the sequences from gene TRPM8 comprise or consist of:

(SEQ ID NO: 859)
CTTACTTTAAGGAAGGTCCCTGGAAGTTGTTCATTTCCTGCATAA
TAGTCCGTTGATTCTTGCATTTGCTGCACTTACGTTCTAGTTCAT
TAAAAGAATCTAGGCTGTTCCTTGGGGGACTCATCAAAAACCCAC
ATTTTTATGTTATTTCAGTTTTAATTCAGTTTTTAAAAATTTATG
CAGAAACCACTGAGCAAATAAAATGCTCAGAAACATTCTGGGTTT
TAACCTAGATTGAATGAGTTTGAATGAGTTGAGCCTTGATCATTC
AACCTTAGTCACGCAGCTCACACCGATAAACTGGTCAGAATACAC
ATCTTTAAGAGAAAAAAACATTCTCAGAGCTGCTTCTATAAAAAA
AAAAATCTCTCAACTTTGGTGGTATATTTGGATCTATTCCCAGTA
TATTCCCAGTATATTTTGAAGCAAACCATATCCTGCCTGAGAATG
GAAGTGGGAAATCTATTCTTAACACAGTGTTCTTTCTTTGTTTTT
ACAG

In some embodiments, the intronic domain that promotes trans-splicing primarily in the heart comprises or consists of sequences from the gene TRPM8. In some embodiments, the sequences from gene TRPM8 comprise or consist of:

(SEQ ID NO: 860)
GTGTTGCTTTTGTCATTTTATTGAGAGTGACAAAATAAAATTCCT
TAATTCGGTCAGATTCACTTCTATAGGGATTTGGTTCCCAGAGTT
ACATGAACTGACTTCTCTAAGCAGATGACTAGAGCATTCTGATTA
AATGCAAGCAAATGAATTCCCCCCTCTGGTTCTGGTTCTTGTCAA
CATGGCCAGATCTGAATTCTGTGGCCCACTTTTTGGTTTCCCCCA
AGTCAGGCATCCATCTGTAGCCCCGGGGACTCCAGATTCATTGAC
TCAGAGCCACCCCAATGTGTCATGGGGCTTTTTTGAGGCCTCATG
TGGGCTCACAAACAGGTGCTCCTGCTCTATGTTGGGGTCAACACG
AATGTTGATTGTCCACAAACCACTTGACAATGGAGCTTCGGGCCC
AGCAAAGGACATGATTCCCTTGTTGTAGGCATTGTCTCCAGAGTG
TTCCTGGGCCCTCCTGCTCCAAACACACATTCATTCCAGGAAGAA
TCTG

In some embodiments, the intronic domain that promotes trans-splicing primarily in the heart comprises or consists of sequences from the gene SEPT2. In some embodiments, the sequences from gene SEPT2 comprise or consist of:

(SEQ ID NO: 861)
AGTACATTCCTCACATTTGTGAACAGTTATGTTTCTAATTATATG
ATGTTCAAGTTCTGTAGTCTCATAGACCTGGATTCAAATCTTGGC
CCTGCTCTGGATAGTACTCTGACCTTGGGCAAATTAGAGTTTCTT
TGAGTACCCTCAAAGATCATACTGCAAACATAGTAGTACTGTAGG
TACTAAACACAGAATGATGTTTGTGAGGTACTTAGCCCAGTACCA
GGCCTTATGTAAACACTTAACCGATTGAATGGTGTCATATGAATA
AGGTCATGACAAGGTTTTTCTTTTTATTTAAAGGTGAATCAGGTC
TAGGAAAATCGACTCTCATAAACAGCCTATTCCTAACTGATCTGT
ACCCAGAAAGAGTCATACCTGGAGCAGCAGGTAAAAACATTCTTA
TGTTACTGTAAGTGTAATTCTACATGAAAGATGCTGAAGACTGCC
TAATTAATATTTTATGTCTTAGCTGTGTGTTTTGTTTGTTCTTGT
TCAG

In some embodiments, the intronic domain that promotes trans-splicing primarily in the heart comprises or consists of sequences from the gene SEPT2. In some embodiments, the sequences from gene SEPT2 comprise or consist of:

(SEQ ID NO: 862)
TAGGTGTGCTGCACCGAGGTCCTTGGATTTGGGTTGTAGACTTTC
TTTGACACGTATCCATGGATCCATCCTCTCTTGAGTTTGTCTGTA
AAATGTAATAAGTAGTTTCTGCCTATATTAAATATTTGATGGAAG
GATTAAGTGAGAATGTTTTACATCAAAAATGCTGTGTAACTAGAA
GATTACTAGGATAGATGTTCGAATTCATCTACTGTCAGAAAGTTC
CAGCACAGGACACCAGCATCCACAAATGACTGTTGAATGAAATAG
GCACACATAGTGAGAGCCTAAGACTTGGGCTGCTGCAAGAATATG
TAGCCCTTTCAGAATATTTTTGAATTCAGTTTTTAAATGAATTCG
TAAATTGCTTTCTTTTTCCCAGGAACAGAAATTATAATTTTTAAC
ATTTGTGTTTTTAACCTTCCCCAGTAGCACAATAATCTTAGCTTT
AACCTTCCCCTTTCTGACTTCTCTGGGATCTTTTTTTCTTAATCC
CCAT

In some embodiments, the intronic domain that promotes trans-splicing primarily in the heart comprises or consists of sequences from the gene RPL15. In some embodiments, the sequences from gene RPL15 comprise or consist of:

(SEQ ID NO: 863)
CAGAGACTCCGCAGGGGGAGAGCCCGCGGTGCGTGAAACAAACCT
GTTCTCTCCTCAGACCTCAAAGACAGCGGCTCCACCGCGGTACGC
GGCCACCGGCTTTGGAGCCTGGACCCCAACTTGCCTCCTCTCGCG
GAGAGACAGTCGCCGACGCTCGCTTAGCCGCCGAGACCTCGCCGC
CAACTCTCTCACCTCTCGAGACGCCCAGGCCGCTCAGGCTCGAAT
CTTGCGGAGCAGGGGGCGGGACAATAGCGGCCGCGGCGCCCCACT
CGGCAGAACTCCGCCACCAGGCGCGATGCCGGAACTACATGTCCC
ATGACGCTCTGGGAGGCCGCAGCTTTCCACCGGAAAGAGGGTGGC
TGAGGTGGGGGAGGAGCCCAAAAGGCATTGTGGGAGTACAGCTCT
TTCCTTTCCGTCTGGCGGCAGCCATCAGGTAGGCTGCGTTGAGGA
TCTTTGCTCTTCCATCCGCCTTTGATCGTCTTCCTCTTCAGCCAT
CCAG

In some embodiments, the intronic domain that promotes trans-splicing primarily in the heart comprises or consists of sequences from the gene RPL15. In some embodiments, the sequences from gene RPL15 comprise or consist of:

(SEQ ID NO: 864)
TCAGGATGCGGGACAGCAAAGGGGTGGAGAGGCGGCCCCCGGGGC
GGGTTGAGCGAAGATGTGATGGCGGCGCGAATTCGAGCTGGGCCC
GGATCCTGGAGGAGGCGTGGGGCTGACGAATGGGGCGCCCGGGAC
AAGGCAGCCTAAAGTGAGGCTGCTGGTTGGGTGGGGTAGATGCAT
TTTCCTCGAGAGTTAATCCTCGGTGGCCGCAGCAGTACCTTGTCC
TGTGATGTCAGCGGCATCTCCTTTCAGGTCCTCCTTGGGGACGCC
GCCTGCCGCAGCCACCCGCCCCCTTGGTGCTCAGTTCTGGTTCTG
TTAATTCGCCCCACCAAAACGTGCCGAGCACCGCTCTGTGCTGGG
GTTGCGGAAGTGACTGAACGCGGCTCCGTGGGCGCAGTGGTGGGG
GTTGGGCTAGCTGTCCCCGGCAGTTGGTGCAGAGCCATTTTCATT
CCCGGCGGTTTCCTTGTTTTTGTTGGGGAACTAAGATGGACGGAT
ACAG

In some embodiments, the intronic domain that promotes trans-splicing primarily in the heart comprises or consists of sequences from the gene AZ12. In some embodiments, the sequences from gene AZ12 comprise or consist of:

(SEQ ID NO: 865)
GTAACGAGCTCTTCTCTTAGGTTTTGGAGTCAGAGTTTGATTTGA
ATCCTACTCTACCCACTCTTCATTCAGTAATCAATATTTATTGAG
CAACTTCTACACAGTGATGAGTTGGGGACACAAATGATGGGCAAA
ACAGACAAAGCTTCTGCCCTCACAGAGCTTAACATTCTTCTAGAT
TTAACAGGGCTAATTCAACTTTCTAAGACCTACCTATAAAATATA
GATGATACATATTTCATAGAATTGTTCAGATAATTAAAAGAGATA
ATATTAAAAGAATGTAGTGCATTATAGTGGGACATATTGGACACT
CAAATGTTAATTAATTTGTCTTCTATTCTAAATATACAACAAACA
TAGCCAATTACTGGTATGGAAAGATAGAGTATGCATTAAGACTTC
TATAATATAATATTAATATGTTGTTGTAATGAATTATACTCAAAG
AACTTTTGAAAATTTATAAATTGAGAAGCTATGAAACTTGCTACT
AATA

In some embodiments, the intronic domain that promotes trans-splicing primarily in the heart comprises or consists of sequences from the gene AZ12. In some embodiments, the sequences from gene AZ12 comprise or consist of:

(SEQ ID NO: 866)
TATTCATGTGATCTGAAATAAGGCAAAAGAAATGTTAAAGGGATT
AGTTGACACAGGAGGGAGATTAGACATTTTGGAAGAAAGCAGGCA
TAGTGAAACACGGGTTGGAAACGTGGCTGTAGTTAAATTGCCTAT
GTTTACATCTCAACTCTCCACTTACTATGTTACTTTGGAAAAAAT
ACTTAAATTCCTCCTACCTCAGTTTCTTCATCTATAATGTTCTTG
AACAGTGACTGATATTAGTAAGTAACCAAATTTTATTTCAAGCAA
GGGATAGTTAAAAACTCACTTTCTAGTAATAATTACTGATCTAAG
ATACATGAAATTCAATTGATAATCTTTATTTAGAGTGCTATATTT
AGTTTTTTATTTTAAACTAAAAAATATTTAAAAGGACTTTTTAGG
TTCTCTTGCATGGTCTAAAGTCCCTCTTATTCTGTTTTTCCCAGG
AGGTCTTAAAAGGTTTTAGCCACAGTTTCTCCTTCCATCATGTTT
TTCA

In some embodiments, the intronic domain that promotes trans-splicing primarily in the heart comprises or consists of sequences from the gene IP6K2. In some embodiments, the sequences from gene IP6K2 comprise or consist of:

(SEQ ID NO: 867)
GTAAGAATCCATTAAAGAAATTAATTTTAAATGTGCATTACCTGG
TTACATAAAGGACTCTAGAACAACTTAGGTTCTTTTGGGACTGTT
GAAACATTGTCTTTAAGGACAGAATGTACTGGGTGCCTTTCTCTG
TACTCTGAATATGCATGTGTATTGCGGTGGGGATGGGGGAGTTGG
GGGCCCTCACTTGGTCCTTCTCTGTTGTTTTTTTCCAGGACAAAG
CCAAAGGCCCCTTGTTAGCTGGCCATCCCTGCCCCATTTTTTCCC
CTGGTCCTTTCCCCTGTGGCCACAGGGAAGTGTGGCCTGAATACC
CCACCCCGGCTCCTCTGCACCCAGAGCTGGGGGCCACCTCAGAAG
TGTCATCTCTCTCTGAGCACGCATTCCCCTGCAGCAGTCGAGGAC
TGAGCAGATTGAGTGATGCTGGGGCAGAGAGGCCTGGGAGGAAAG
GTGTTCAGCCAGTCGTTTGTAAGGCGCTCGTCGGCACCTGCTGAA
ACGC

In some embodiments, the intronic domain that promotes trans-splicing primarily in the heart comprises or consists of sequences from the gene IP6K2. In some embodiments, the sequences from gene IP6K2 comprise or consist of:

(SEQ ID NO: 868)
AAGGCCCTGGCAGCCATCCCTGGAGCCCTCACTCCCTCCCACTCT
GTGCTTTCCCTCCCTCTCCTCCTTCTCCTCCTCCTGGCCCTCAGC
CCAGCATCTCACACCCTCAGTTTTCAATCCCTGGTAAGATTGCTC
AATTCTGTTTTGTTGTGTGGATTTGAGTTTGATTTTGGTAGAAGT
GTAGTTGTGTTTTATGTTCAGGTGTCTCTTGATCACAGTAACCCA
TAGTCCCCCACTGGGGGGACGGTGGGGGAAGACTTTGGGAGGATT
TTACCCAGAATACTTGCCGCCTGCTTTTTGTCCTCCAGGAAACCA
GAAGCCCGGGTAATTAGGTAGGCCGGGGAGCAGCGTTGAGTCAGA
TGGAATGCAGGAGTGGGAGGCCTGTGCTGATTAGCCCAAGCCCTT
TGCTGTTACAGAATGTCCCCAAAATTGCTATATGAAAGCAACCCT
TCGATTCACATATGATAATTTTTTATTTCTTTTTATAATGTATTT
TATA

In some embodiments, the intronic domain that promotes trans-splicing primarily in the heart comprises or consists of sequences from the gene MST1. In some embodiments, the sequences from gene MST1 comprise or consist of:

(SEQ ID NO: 869)
GTGAGCACTAGTGACGCTTCCCCCATGACCCTGCCTCAGCCCCCA
CCCAAAGGCTGGCTCCCTTAACCCCAGTGAACTTTGTCTTTCAGC
TGATGACCAGCCGCCATCAATCCTGGACCCCCCAGGTTAGGAGTT
GGGCCAGTTATGGGTCAGGCCCTTTAGCCCACGACATCCACACAG
TCTGGGTTTCATCCAGCCCACCCCATCCTACAGACCAGGTGCAGT
TTGAGAAGTGTGGCAAGAGGGTGGATCGGCTGGATCAGCGGCGTT
CCAAGCTGCGCGTGGTTGGGGGCCATCCGGGCAACTCACCCTGGA
CAGTCAGCTTGCGGAATCGGTGAGGCACAACTGCCTGTCTCCCAC
AGAGAGGAGCTGAGGTTGTGTCCTCTGTGGTTATCCCACTGGGGG
CTGGGAATCTATCCCTGCCCCCAGAGGTCCTAGCCAGAAGATGGC
AGGTCTAGCATCTGTCCCAGGAGTCTGTTACCTGTCCTAATTCCC
CACT

In some embodiments, the intronic domain that promotes trans-splicing primarily in the heart comprises or consists of sequences from the gene MST1. In some embodiments, the sequences from gene MST1 comprise or consist of:

(SEQ ID NO: 870)
GGAGGCTGGCGGGCTAGGGCTGAGTGGAGCGCCTGCTTAGAGACC
TTCGGGAGAACTTCTGCCGGAACCCCGACGGCTCAGAGGCGCCCT
GGTGCTTCACACTGCGGCCCGGCATGCGCGCGGCCTTTTGCTACC
AGATCCGGCGTTGTACAGACGACGTGCGGCCCCAGGGTGAGGCCC
AAGCTTGGGGGCTACAGAGCCGGGCTGGAAGCCTGGAACCGGAGG
GCCGGGGCGAGGTCTCGGCCTGATGGCTGCCCGCACCCGCCACAG
ACTGCTACCACGGCGCAGGGGAGCAGTACCGCGGCACGGTCAGCA
AGACCCGCAAGGGTGTCCAGTGCCAGCGCTGGTCCGCTGAGACGC
CGCACAAGCCGCAGTGAGTCCCTGGTGCTCCCGGCCCCGCCAGGG
CCCTAACCCTGGGGCGGCATGCTTTGGTGTCTGGGACCAGAGCCT
GGAAATGGTTGAGACTACCCTGCCACGATTTTGCTCCCGCTTCCG
CCTA

In some embodiments, the intronic domain that promotes trans-splicing primarily in the heart comprises or consists of sequences from the gene ITIH3. In some embodiments, the sequences from gene ITIH3 comprise or consist of:

(SEQ ID NO: 871)
ACATCCCATTGCCACCAGCCCCATAAAGGGCTTCCCTGGACGTGC
CCGATTTCCAAAGCCTAATCTTTGTAGTCACATCTCCACCTCTAT
CTGCCAGTGTTGGGGCCCCTCTGGGGCCCAGCAGCGCTAAGACAG
GAGGAAGGCACGGGGCTGGGGGAGAAGGAACCCCTAGACCCTGCT
CACCACTGCCCTTCGGCTTTTCAGCCACACCGGTGAGCCCCGCCA
TGTCCTACCTGAGTGAGTACATGCTGGCAGCTGCCACTCCTCCCA
CTGCTTAGAGCCTGCCCCTGCCACATGTCCCTCCACCGTGGCTAC
TCACTCAGCTCTCCGGGATGGGCAGATGGACAATGTGCCCTGAGG
AGATTGCGGGGTGGGAGGGCAATGGCATTAGCGAGCGGGAAGGGG
AGGGGAGGGGCATAAGCTGAACTCCTATGGGGGTCGTCGGGCGCC
TCCCAGGGCCACTTGCTTAGCTCAGCCCTCTCTCCCTTTGTATCT
GCAG

In some embodiments, the intronic domain that promotes trans-splicing primarily in the heart comprises or consists of sequences from the gene ITIH3. In some embodiments, the sequences from gene ITIH3 comprise or consist of:

(SEQ ID NO: 872)
TGAGTCCCTGGCTGCTCCTCGGGAAGGCTCAGGGGCCTGGGCACG
TGCTCCTGCCTGTCTCGGAGTCGAGCCCATGGAGGCCAGAAGGCT
GGGGGAGGCCCCAGGTATGATGAGAGGCCCCGGGAGTCAGCTCCT
TCTCTAATGTGTCACCAGTGGACGGGGATCCCCACTTCATCATCC
AAATTCCGGAGAAAGACGATGCCCTCTGCTTCAACATCGATGAAG
CCCCAGGCACAGTGCTGCGCCTTATTCAGGATGCAGTCACAGGTG
AGGCTTGTGGGCTAGGGCCGGGGCCAGGGGGCTCTTCCTGGGAGG
GCTTGGGTCCCCCGAGGTAGGCACAGGGAACAGGTTCTCACTCTG
CCCAGTAAGGCATCATCTTCACATGCAAAGAAACCCCTGGGGGTG
GGGAGTGCCCAGGGCCACAGGCTTGCTCAGCCCCACATGCCAGTG
CTATTGGGAGATGATTAGCGTGAATGGACCACCTGGGCCTAGGGT
AGGA

In some embodiments, the intronic domain that promotes trans-splicing primarily in the heart comprises or consists of sequences from the gene ITIH4. In some embodiments, the sequences from gene ITIH4 comprise or consist of:

(SEQ ID NO: 873)
GTGACAAAGTAGGAGGTGGGGGGAGGGGAGATTGACAGATTGGGC
TCAGTCCTGGCTGTGGAGGGTAGTGCCTTGCCCAGCTCCTCTGGC
TGGTGACAGCCCTCGGGCCTCAGTTCAGTTGTATCTGATCAGGAC
TTGACCACACAGAGGCTTCCTTTTCTCCAAGAAGAGGATGGAATA
GACAAGGTAGTTTGAGGGTTTCTGCCAGGGGGAGGCATGACATCC
CTGGGGGGGAGCTTGAGGGAGGTCCATTATTGTTCATCTCCCAGC
TGTTGGGGGAGAGGCTCCCCAAGTTGGCCGGGTGTGGGGAGAGGG
GACATAGCTGCCCTCTCCGCACCCACTCCTGGGTGGATCTCAAGC
CCTGGCTGTGAAGACCCACACCCCACCTGAGAGAAGACTGCATCT
GTCGATTTCTCCAAAGCTGGAGCTGCTGGCTCCCGGATGAATTTC
AGACCTGGGGTTCTCAGCTCCAGGCAACTTGGACTCCCAGGACCT
CCTG

In some embodiments, the intronic domain that promotes trans-splicing primarily in the heart comprises or consists of sequences from the gene ITIH4. In some embodiments, the sequences from gene ITIH4 comprise or consist of:

(SEQ ID NO: 874)
TGAGGAAAGTGAAGCCGGGTCTCCTGTCTCCCCAAATTCATTGCA
CCTACGATATAGTCCAGGATTGGGTAGCAAGATTAAACACAAGCG
CTTCAAATTGATGAAAACCACCCTGACGGTGTCTGTCTGTGTCTC
TGTCTGTCTGTCTCTCTCTCTCTCTTTGTTTTATCCAGAAAGTAG
AAACAGGAATGTCCACTCAGGTAAGCAGCGAACATCCTCAAAGGC
TGTGCTGGGTGGTGGTCTGCTAACACAGGGTCCAGGGCACCAGCC
CTGTGAGGTTGCTGGCCAGGGCCCAGGTCCACCCAGGCCTCTCCA
GCCCATCCCTGCTGCCAGGAGGCAGTGTAATCTGACCCAGCATGC
TGGTGCATTTAGTCGCCTGTGCATTCAAATATTGGCTGTGCCTCA
GGCCCTGTCCCCAGCACTAAGAATACAGTCCCCATCCAAGTGGGG
CCACAGGAAGGTGGGTGCAAGATGCAAACCCAGGTCCCACTGATT
TCCA

In some embodiments, the intronic domain that promotes trans-splicing primarily in the heart comprises or consists of sequences from the gene IL17RB. In some embodiments, the sequences from gene IL17RB comprise or consist of:

(SEQ ID NO: 875)
TTTGTGTTAGAAGGGGAACATTTTAAAGAACCTGAGGAAAGTACC
ATGGACTGCATTCAGAGCTTTCTTTTGTTTATCTCCTTAGCATAG
CTACTCCTTAACCTGGGGTAGCCCAGTAAATACTCTAGGCCGTCC
TAAAAACCTAGAGTTAAACATTAGAGTGTGAAGGGGCCTCACATC
GGTCCCAAGTCATCCCCTGCTTGCAGCAGGGAAGGATGACATCCA
GAGACAGATGGAGACCCACCTGTGGTCTTCAAGTGAGGTTATGCA
CTGGCTTGAGGATCAGGTATACTGATGGCTAATCCATGGTCCGTC
ATAGCCTACAGTGGGACAGCTTAGTTCCCCCCTAAGCTGTGAAGC
AGAACCAGTTCATCTTTTCTTTGAAAGTTTGTCACTTGTATATGC
ATTTAAGGGGAAAATGGTGTTCTGCCTAATTTGGCAAAGGAAAGC
TTGTGGTTTGCCGGCTTCAACGTGACGCTTGGTTTTTTCTCTCCC
ACAG

In some embodiments, the intronic domain that promotes trans-splicing primarily in the heart comprises or consists of sequences from the gene IL17RB. In some embodiments, the sequences from gene IL17RB comprise or consist of:

(SEQ ID NO: 876)
TAGGTGCATTAGAGAATGGGTATTTGGGGCTTTACATTGAAAGCA
CAGTTGGACTTTTAGGAAGCCTAATATAGGCAATAGGTCATCGAA
GACCAGACACAGGGTGACCCATTGCTTTATTGGCTGATGCAGATG
AAGTCTCTGTCTGCTCGAAGCAGAACTCTGGGGCTCTGTGGGAAT
GAGGCCGTGTTTCAGACAGGCTAGCCTCTTGGCCAGTCAAGATGG
GACTTGCCATACAAGGTGGAAAGTGATGCCTGCCACTCTGCAGAG
GGAGATCAAGGCCGTGGAAGGAAGGTCACTTAGAGTTGGGACCGA
GAAGCAAATGTTTCATAGGAGAGCGGGTGTTTGAGATGGTGGCAT
TTGAAGAATGGGTAGGCTTCTGCCCAAAAAATGTGGGTAGGAAGA
GTGAGCTGGAGGAGAGATGGCACCAGCAAAGGTTAGGAGGTGGGA
AAGTGGGGGCTGTGTGGGCCAGCTGGCTGGAATCCAGGGCCAAAG
CCTG

In some embodiments, the intronic domain that promotes trans-splicing primarily in the heart comprises or consists of sequences from the gene DNASE1L3. In some embodiments, the sequences from gene DNASE1L3 comprise or consist of:

(SEQ ID NO: 877)
GTGAGGACTGCTGTGTCAGACGAATTCCCATCTAGCAGGGGCAGGCTCT
GGTCCCTGAGTGGGATGAGTACCTCTCCAGGGAGGGGGAGGAGGAAGGG
CATGTGCAGGCTACCACCACTTACAGCATTCTTTAAGGCCTTTCTTCAG
CCCTAAGTATCCCTTTTGCTAAATCTGCCTGGGTACCCTGCATGTGGGC
CTTGGGAATTGCCACACTTTCCCATTAATAACAGATGCCAACATTTACT
GAGTGAATAGCTAGTGCCAGGCACTGTGCTGAGACTTGACATGGACAAG
TTTATTTAATCCTCCCAATAATCCCTTGAGGTAGCTACAGTACCTTTCT
CATTCCCACTTTGCAATTGAGAAGTGTAAGGCATAGAGCAGTCAGGATT
GTTTCAGTTGCAAGGGACAGAATCTTAACATGGCTCATGAAAAAAAGGA
GCAATTGATTGGTTTGTGTAACTGAAATGTCCAGGAGTACTTCAGGCAT
AGCTGGATC

In some embodiments, the intronic domain that promotes trans-splicing primarily in the heart comprises or consists of sequences from the gene DNASE1L3. In some embodiments, the sequences from gene DNASE1L3 comprise or consist of:

(SEQ ID NO: 878)
CTGGAATGTGCCTGCGAGCTGGAATGTCCAGCACAAATATCTTGGTTAA
GGTACAAGGACATAGAATGTACTATGTGCCTGTGAGCATGTCTAACAGC
TACATAGGATAGAGCTTAACAAAGAGTTATTAGCACAAAGCAAGGAGAC
TTGAAGGAAGTTAGTCTTTAAAGGAAACTATTATTTCTAACACTTATGA
TTTATTCTTTAATAAGAAGGGAAACTTTGAAGAGGAAACTTTTTTACTT
TCTACAACAGTCTACCAGCTTTCTCTTAGCCATGAAGGGCAACTGGGAG
AAATGAGTCTTTCGTGTACTAAAAATAGGCAGCCTAATGACCCCAAGAT
CATTAAAATCTCGGCCACTGGCTGGACCCTCTGGGAACTGGGGACCCAG
ATAATGCAGCCTGTGCCTTCTGGAGGGGACAGCTGGTTTCCCTCCAGCA
GTGTTTATGAGACCTTGACAGGGGAAGTGTGTGACCCCCGCCTCTTTCC
TGTTTCTCA

In some embodiments, the intronic domain that promotes trans-splicing primarily in the heart comprises or consists of sequences from the gene MYLK. In some embodiments, the sequences from gene MYLK comprise or consist of:

(SEQ ID NO: 879)
GTGAGTGCCCCCAGGAGCTGCCCCTCCAGGCCATGCCCCCATCCCCACC
TTCCTCCTGGCCCCGGCAGCTCCAGCCCAGCAGCCCTAGATTGTTTGTG
TCCTGCAGCTGAGGCTGCCAAAGCCAGAAGGGAGAGCCTGGAAGGGGAA
TGAGGGAGGGAGAGCAGGGGAAGGAGGGGGTGATTTTTTTTTTTTTTTT
TTTTGCACACACCCTTATAAGGCTACTGAAGTCATTACCGATGTAATAA
ACCAACTAAGCAAGCGAGCGCCTCTCACCAATGTTCCCATCTATAAACA
CAGCCTGGCCGGCCTCCCCCATCCCCTCCCGCTCCCCTCTACCCTCCCA
CCCTCAGGGATTTGCTCTCCCCTGAAGTTTCTTTCTTACCCAGCCCCTT
TTCCTCCTGCTACTTTCCTTTTTCCCTACACTGGTTGCAGGGGGAGGGA
GCCTGCCAGCTGCGAGGACCTCTGTGGTGGAGGAGTTGGTGCTGATGGT
GGTGGTAGT

In some embodiments, the intronic domain that promotes trans-splicing primarily in the heart comprises or consists of sequences from the gene MYLK. In some embodiments, the sequences from gene MYLK comprise or consist of:

(SEQ ID NO: 880)
TGAGTGGGGGTTAAGGGTGCTTCCATTCCCGAGCAAGAGAGGGAAAGGA
ACTACTTCTGTGTTAAGGGATCATGGAGTCAGAGTCAAGGTCACTGGGC
AGGCCCACCACCAGGGCCTATCTGTCTAGACCAACCTGCTACATCACAG
TTCCCAGCAGGGTTAGCCTTTCTCCTTCCCCTTTTATTGGTTGAGTGAG
TGAGTGATTGATTGAATGATTATATTATATACTTACAGAAAAGTGTTCC
TATTTCTGGATGCCTGCCTTAGCTGTTGTCCAGACATTCTTTTTTTCTT
CTTCTTTTTAAATAAGAGCTGAGAAAATAAAGGGTGACCACACAGTGAG
AGGGTCCTGCCCTAAATCTAAGGCTGGGGTGAATGTCTCAGTCAAGCCG
GCGACTTCCATCTTCCCCGCTGGCCAGCAGCCTGTGATCAAGTCCACAC
TGAACAGGCCCTTTGCCCAGAGGGCCTCTTGATCTTTATTGATCTTTGG
AATTCTTCA

In some embodiments, the intronic domain that promotes trans-splicing primarily in the heart comprises or consists of sequences from the gene CDV3. In some embodiments, the sequences from gene CDV3 comprise or consist of:

(SEQ ID NO: 881)
TCGCCGCGCCCGGCAGCGTGAGCGCAGAGCCGGCCTCGACCCCGAGCTC
GGAGCCCCGCGGGCCGCGCCCGCCGCCGGCCCCACCCATCCGGGTCGAG
GAGGCCGAGGCCATGGCTGAGACGGAGGAGCGGAGCCTGGACAACTTCT
TTGCCAAGAGGGACAAGAAGAAGAAGAAGGAGCGGAGCAACCGGGCGGC
GAGTGCCGCGGGCGCAGCGGGCAGCGCCGGCGGAAGCAGTGGAGCCGCG
GGTGCGGCGGGCGGCGGGGCGGGCGCGGGGACCCGGCCGGGTGACGGCG
GGACCGCCAGCGCGGGGGCTGCGGGCCCAGGGGCCGCCACCAAGGCTGT
GACGAAGGTGAGGGGCCGGGAGGCCGGCACTCGGGGCCCGGGCCGCGCG
CCGGCGCCCCATGTGCCCGCCCCCGCCTGCCGGGGAAGCGTCCGGGAGG
GGCCGCGGAGGCCGGGCGGACGGGCGCGGGCCGCCACGTGACGCAGGCT
CTCCACCTC

In some embodiments, the intronic domain that promotes trans-splicing primarily in the heart comprises or consists of sequences from the gene CDV3. In some embodiments, the sequences from gene CDV3 comprise or consist of:

(SEQ ID NO: 882)
TCTGGGGCCGCAGTGCCCAGCACAGAGCAGCGTTTATCGGGTGACGTCT
AGGCTGAGTAATTCCGTGTGCTGACCTCAGCCCAGTAGTTTGAGCTGAA
TTTTCGCTTTCAGCAGCCGCATTTGTAGCAGCTCTTTTGTGATGACTTA
GTCTCTAAGCCCGTGAAAGAAAAAGAAAACTCTCGGTGGCAAGGTTGAA
GTAGAAATGTAGTTGTGAAATTCCTATTGACTTCTTCCTTCGGCTTTTC
TTAGTTGCTTAAAGTAACAGCTCCCATGCAGGAACCCAGCGGAAGTATA
GTGTTAGCCTACGAGCATGTTTGTCCTCCATTTAGCCTAGTTTGCCCCA
GAACGAAGCAAGGGCTTTGACCAGAAGACAAGTACATACTTAGTTTAGG
TTCCAGCCACTTGATCCACTTTTCGTAGTGTGTTATTTGCTTAGTTATA
TGTCTACAAATAGCTTTAATTGATCAAATGGCGTTTGTTTTACAGGACG
AAGATGAAT

In some embodiments, the intronic domain that promotes trans-splicing primarily in the heart comprises or consists of sequences from the gene PLSCR4. In some embodiments, the sequences from gene PLSCR4 comprise or consist of:

(SEQ ID NO: 883)
GTATGTATAATTTTTGTAGTGCTCAGCAGTATTTTAATCAGATGGAAAT
TTGCATTATCCAATATCTAGTTCCTTCTTTATATTGATCATGTTTTAGT
GGCTCAAAGAAAAAAAAACTGCTCTATTACCCTATCTTCAAGAAAGTAA
TAAAGTGAATTATTTTGTATAATTTTATCCTTCTATACATCATATAATC
ATAAGCTCTTAGATTTGAAAAGGGCTTACATTTTATCTGGTCAATAGCC
CTACACTTTACCCATTGTTCCTACTCCACATTTACCTTTCAAAGATGAG
TAAAAAAAGGAGCCAAGAGACTAAATGAGTTAGCCAGGGACACCACAGT
CAGCTAATCACCTCCAGATCAGGAAATAGCATTTAAACCACAGTCAGCT
AATCAGTGGCAGAGCTGGAAATAGAATCCAAATCTGCCAAATCGTAGTG
CAGTGCCCTCGCTTTGGAAGTAAGAAGAAAAGATCTCAGTCAAAATGAT
GCCTGTTCT

In some embodiments, the intronic domain that promotes trans-splicing primarily in the heart comprises or consists of sequences from the gene PLSCR4. In some embodiments, the sequences from gene PLSCR4 comprise or consist of:

(SEQ ID NO: 884)
TAAAATAATATAAAATGGGGACACTCCATGTGAGGTTCACCCTGGCTTG
TTTGTTCTCCTTAAGCCACTGAGAGACTTCCCTTTTCTATGGCTGGTCC
GTGTTAATCGTTCTGAACTTAACTAAACAACTACAGGCTATGGATGACA
TGTAGAGAGACCAGTGAGTAGGGACTTATCTAGGAACAATCTATAAGCA
CAGCTTCTTCTGATGTGATTACTACAGCAAGTAGATGTTTTGTTTTTCT
GATATAGCAGACAACTTTTTTAAAGTGGTACTGAAAGAAAGTGTTTTTC
TAATATGGACAACTCTGCTAATATCCATCCGATCAGATGCAGATAAGCT
AATTTCAGTTGGTAAAATTTTTTTGATTCTATTGACTTTCAAAAATATA
TTTTGGTATCAATTGATGCATTTTAATATCTATTTCATCAGTTTATTTT
ACAGTGGTATTTTAGTTCTGTTATTTCTGTCTTCTGATTGCTTTGTTTT
ATCCCAACA

In some embodiments, the intronic domain that promotes trans-splicing primarily in the heart comprises or consists of sequences from the gene BCHE. In some embodiments, the sequences from gene BCHE comprise or consist of:

(SEQ ID NO: 885)
GTGTGTCTGTTTTTAGTGTTTGGTTATTTAATAGATGATGGGTTATGTA
AAAAAGGGATGAAGTATACTCTCCAAGGCACGGTGATGGAGTTACTATA
TGTATCTCTGACTTTAAAATAACTTGGTTTAATGAACACATTATTTTTA
AGACTTTCTTCTATCAAGTTTAGATATGTTTTGAACTCTTACTATATGA
CCAGTACTTTAGTAGTTAAATAAAGAAAACTAAAATATAGATTTCACCC
TGTGGAAATATAAAATAGAAAAAGGAAACTAAAATAGAGCCCTACTTGT
TATTCAGATAATCTTTGTGGAATTAATTAATGGACAAAGAAATAAATAT
ATCAAATAATCATAATGTATGGAACTAAAAAATAAGATGCATAAATTGG
TAAAAACAGATTTATTGATAAATAATAGAACCTTGATTTAAATGGAGAA
AAAAACTCCGAAGTTTACATATTTAAAAACACAATAAAAAATTTCTAAA
TATTCTGAA

In some embodiments, the intronic domain that promotes trans-splicing primarily in the heart comprises or consists of sequences from the gene BCHE. In some embodiments, the sequences from gene BCHE comprise or consist of:

(SEQ ID NO: 886)
TTTCCTGGAGTAATGAGATTATTACATAATAAGATTTGTAATTAAACTT
GGTCTTAAACTCAAGCAGTATTAATAAGAGTATATAGAATGAAATAGAT
GCAAAAGATGAGTAAGTGATATGATTCCTTATTGTGAAATTTTTGGGCC
TAGGATAAGGGAAGATAATGAAACAAAAGTAACACCACCCTTTGAGCAC
GTTTAAGTGTGAATGAAGATATATTTTATTGAAATAAAAAATTTCTTCT
TCCAATATGTTAAGCAGAAATGCATATAAAAAATTCAACTTTAAATTTC
AATTCTTACAATTTTAAATAACATAGTGGTTCTCAGTGCTCAAACCACT
AAGCCCAGTTCACATACGTTTCATATCATCAATATATATCCACATTTTT
TCTGTCTTAAATTCTTTAAAGCTCTTGTGAACAGTGTTAGAAAACAATA
TTCTTTTTAATCTAACAATGATTTCAATTTTACTATAATGTCTCTATTT
TATTATTTA

In some embodiments, the intronic domain that promotes trans-splicing primarily in the heart comprises or consists of sequences from the gene MASP1. In some embodiments, the sequences from gene MASP1 comprise or consist of:

(SEQ ID NO: 887)
GTGAAGTTCACTTTATAACCTACAGACAGTGACACCTCAGCTCTGCACT
GGGCCTTCCTGCTCCGTATTGTTCCCTTAGGGATTTTGAGGCTTGGGTG
GGGGTTGGTGAGTGGGAAACTAGGAGCCCAGACCCACCTCCAAAGTTCA
ATGGGACTTTGAACCCCAGAAAGCTTGATTTGTTTGTTCTACATGTCAT
GGACTCTAGCCTCTAAACACGGTTAAGTTTTTCTGGTAGCTTATTAAAG
GCTTAGCTTATGAAAATTCTCCCAGAAAACAGCAAGTTTAGACACCCCC
ATGGTGACAATCCAGCTGTGCACCATGAGAACTGTACACCAAATTGGCC
AGAATGAGCCAGGCTCTTGAGAACCACAGTCCTTGCAGTAATGAACTTG
GCAGTTACACTTAGAAGTTCATATTCCTATCAGTTCAAGGAAAAAAAAA
AAAAACACTTAACTAAAAGTAAGGAGCCATAGTTTCAAGTCTTGGCTCC
CACTAACTT

In some embodiments, the intronic domain that promotes trans-splicing primarily in the heart comprises or consists of sequences from the gene MASP1. In some embodiments, the sequences from gene MASP1 comprise or consist of:

(SEQ ID NO: 888)
AAGGGGTTTTAGAGAATACCAAGTCCAGCCTCCTCATTTTATGGATAGG
AAAACAGAGGCTTAGAAGGGGAGCAGAGAGGAGGGCTGGCTAAAGGCAC
CATAGCACATAGTGGACTAGGCTGGGCTTTTTATTTATTCGCTGTTCTT
GATTGAGCACCTACTATGTGACAGCATTGCTATAGGCACTGTGATATAG
CAGTGACCAAGAATGAGATTCTGCTCTCATGGACCCTAGGTTTCAGACT
AGTTGAGGGTGGGACAGAATAGAAAACTAAACAAGTAAGCCAGTAACAC
ATCATAGAAACCAGTGCCCCTAGCTCTGCTGCAGGTAATCTGTAGCCCA
GCTGCCTTTCACGTCTTCCTCCCCACCACACCACCTTTCTCTGAGAAGA
ACAATCCAGTCTTTCCTCCACAGAAGAGGATGATGGAGCTGCAAGCTGC
ACCCCTCCAAACACCCCACAGATAGCTTCTCTGTTCTCACAGTATCTTC
TCTCCTCCA

In some embodiments, the intronic domain that promotes trans-splicing primarily in the heart comprises or consists of sequences from the gene IL1RAP. In some embodiments, the sequences from gene IL1RAP comprise or consist of:

(SEQ ID NO: 889)
AACCCAAAAGGACCTGGCCCCAGAAGCTTCTCTCTAAACTGATACAGCA
TCCTGGCTCAGAAGTTTAAGCAAAAGAGACATTTTGTTTTGAAGAGAGA
TCTATAAAAAGAAGCCAGTATTGGGATTTGGTCTAGACAATGGGATAGT
GTGTAGCGTAGATGGTATAGTATAGTGTTAGAATCTTACCATTAATACT
TTGTAATCACTGCATTATAGGTGGCTCAGCTTATCAACCTTCCTCGCCC
TCACCTGTAGTGGGGGTCTGTGGTTGCTTACTTGTCTGATTCCTACACA
GTCCATAGCTCTTGAGTCACTCTCAAATGCTTTGATTTTCTTCTCTCTA
TTTCTAGTATAAGTCATAGTAGAACAGAAGATGAAACAAGAACTCAGAT
TTTGAGCATCAAGAAAGTTACCTCTGAGGATCTCAAGCGCAGCTATGTC
TGTCATGCTAGAAGTGCCAAAGGCGAAGTTGCCAAAGCAGCCAAGGTGA
AGCAGAAAG

In some embodiments, the intronic domain that promotes trans-splicing primarily in the heart comprises or consists of sequences from the gene IL1RAP. In some embodiments, the sequences from gene IL1RAP comprise or consist of:

(SEQ ID NO: 890)
TCAGACTGTGTCTAAAACTTTCTTTGTGTCTCTCACCTAACACAGAATC
CCAGAAAATCCCTTCAAGGATGTTTTCAATGGTCTTGAGAAAAGTAATG
ATGAGTTATCACACATGAAATCATGAAATCAGGTCATTCAGAATCCTCC
TGGCTCCTAGTCTATAGAGTGTGTAATATTTCTTCAAAAGTGAATAACA
TAGCACATAATTGTTACATTTTACAGAGACTTAATGCTTTTTTAAAGCT
GATGAAAAACAGGATTCACCTCAAACTTTCATTACAACTTTAAAACTAA
ATAGCAAAGGCCTGAAATGTATATGCTGCGTATGAAGTACTAGTGAATC
AGTTCAGGGTAATTCATTGCTTCTGCTCCTCAGAAGCCAGTGTGAAAAG
GAAAGACTGAAGTACATGATGTAAGGCAGTTTCCCAAATTGAGTTCTCG
GGAACACTACTAAGAATCAGTATTGTCCTGCAATGCATTATTGTACTCT
AGAATCAGA

In some embodiments, the intronic domain that promotes trans-splicing primarily in the heart comprises or consists of sequences from the gene PI4K2B. In some embodiments, the sequences from gene PI4K2B comprise or consist of:

(SEQ ID NO: 891)
ATTTAGAATTCAAAGAAAGAGTCTTAACAACTAAAAAAAAAAAATGAAA
AAAAATTATTTATTGTATTTTATGTCATAAAAAGAATCTGGAAAAGTTC
AGATAAAATTGCACTGTTACTGAATAAGCAGGATAGGTTTAAAATTTGG
CCTCCATAATTAAATTCACCTTAATGAATATTTTTAGAAAAACGGCATT
CTTTTTCAGAGTGTCACCTTGAAGACATGTGTTTATTCTTTTTTTAAAT
TTTCCCAAAATAGCTGCTATGCTCCTTGAAAGTCTAGGGGTAAGCATTT
TTTATTTGCTATGTATTCTTTATGTGATTATTTAAAATTAGTGTATAAA
AATGGTTTTCTTGATAAATGTGGCTTATCTAAATTAGTGTTAAGTTTTC
TTATTGTGTTTACATGACATATTTTTTGACAGTACTGCTGAGAAATATA
AATATTAATCCCCTTGTTCTTGTACTTTCTTTTCTAACTATAGTTCTTA
AAATTATAG

In some embodiments, the intronic domain that promotes trans-splicing primarily in the heart comprises or consists of sequences from the gene PI4K2B. In some embodiments, the sequences from gene PI4K2B comprise or consist of:

(SEQ ID NO: 892)
TATTGAAGTTCTCTCATTTGTTCACGTAAATCTTGTTCATTTTCA
AACTATATTCTTGTTATCCAAATCAAAGCGGTAAACTTTTGTTGC
AGAAGTCTTGAAGCCACTTTCTCTGTCTGTCTTGGCAAACAGATG
TAGTATTGACTCTCCTGGGCAGAATTCATCCTGTGTGGGATAGAT
TCTGACTCATAGCAGGCTCCTAGATCAGTTCCTGTCCCCTAGGGA
GGAATTTCCTAGGGGCAGGGCAAAGTCAGATATTGGAAATGTTAT
CTGCATAATGTGAGTAATTAAGGCTTCATTGTATAAATTATGTTA
GGTGCTTATTTTGACATTATGTAACTCTTAGTGTAGTCTATAAAT
ACACTAAACTCTTAATTTTTCCTGTGCTATAAAGGAGCAATGACG
TAGACCAGGGATGTCCAGTCTTTTGGCTTCCCTGGGCCACACTGG
AAGAAGAATTGTCTTGGGCCACACATAAAAACAGTAACACTAATG
ACAG

In some embodiments, the intronic domain that promotes trans-splicing primarily in the heart comprises or consists of sequences from the gene SULT1B1. In some embodiments, the sequences from gene SULT1B1 comprise or consist of:

(SEQ ID NO: 893)
GTATACTCCAGACTCCATGTCAGTTTTTTAGTATGGCCCTATCCC
TTTACCCACTCACATAGTTTATTTTTTAAAATATTTTTTGTTTCT
TTAACCATATACTTGCAACAATCTATACTATACCCACTCACGTTG
TACGTAAATCTTCAAAAGCCATTAAGTTTTAGTCTGAGTTTATAT
ACTCAAGTTTTCCAAACTTGAGCAGCAACAGAATCCAAACCTCAG
CACAATATTACTACTTTTTTTTCCAACTCTCCCTAGATCTTCAGG
CCTTCTGTAAAAACTGAACTTAAGGACGTTAGTCCTTATTTGAAG
ATTTGGTATAATTCTTTGTTGTAACATTTGTTCCTGCTTATCTGC
TTTTGGGGAAATTTTATACTTCAAATTCTATTTATTTAATAGCTA
CAGTGCTATGTAGATTATGTGTTTTTTTCTTATCTGACTTTTGAC
AATTTGTGATGTGTGAGAAACTTTTCTAAGTTGTTGACTTTATGA
ATGA

In some embodiments, the intronic domain that promotes trans-splicing primarily in the heart comprises or consists of sequences from the gene SULT1B1. In some embodiments, the sequences from gene SULT1B1 comprise or consist of:

(SEQ ID NO: 894)
TAATGAAAATATAGATAAAACAGCTGATATTATATACAAAGTTAA
CATCATCTATGCTACAGTACCTAATACAACACGATAGCCAGTTTT
AGACTTACTAGTTATTGATACCTCTTTGGCATGAAGAAAACATTT
AAATAGTCATTTATAGCAATGCACAGGTATAATAAAACTCCAAAT
TATTAAAGAGGGTATTATTATCTCTCCTTTTGGAGATGGAGAAAG
TGAAGTACAGAGATTCTGAGATACTTTCAAAGTGTCTCACAATGC
TTAAGTGATAGAGTTAGCATTTGAACTCAGGGTGGCTAACCTCAG
AATGTGTATCCTTGAATGTTACACAATACTGACTCTAGTAATGTG
GTGAAATGCCTGTGATTGTTTCAGAATTTATCTGCATTACATAAT
GCTCAAAAATGTCTTGAAAAACTGGTTGGCAAAATAAGGAAACGT
AGCCTTTCTGGAGACTCTAATATTTCCCTATACTTAACTAAATTT
CTCA

In some embodiments, the intronic domain that promotes trans-splicing primarily in the heart comprises or consists of sequences from the gene CXCL2. In some embodiments, the sequences from gene CXCL2 comprise or consist of:

(SEQ ID NO: 895)
GTAAGTCTCGCCCATCGCTGCCGCTGCCACTGCTGGGGTCCCCGA
CTCTCCCGCTGCCCCAAACCCTGTCCTCAGCCCGACCTCCTGTCT
CACGAGATTCCCTTCTCTCTGCAGAGCCACACTCAAGAATGGGCA
GAAAGCTTGTCTCAACCCCGCATCGCCCATGGTTAAGAAAATCAT
CGAAAAGATGCTGAAAAAGTAAGTTATAATTTCCATGTACACAGG
CGACTGGAGCTGTTGGTCAGAAATACTGGCGTCTGCCCCCTAAAA
AGTAAATCAGGAAAACCCAGGGTTAGCTGCAGGACTGAAAAAATT
ATTATTTTCACAAAGTTGCCATTAAGGTTATTAATCTGTTCTGGT
GCCAGAGGATATTCCCAGTGCCCAGGGTCACCACCCAGGTTCTTC
CCTCCTGCCAGTGAATGTAGGTAAAACTGCTTTCATGTAAGGTCT
CATTGGAGTTTGCCAGTTGTGCTGAAGTGTTTTCCACCTGTTAAA
AAAC

In some embodiments, the intronic domain that promotes trans-splicing primarily in the heart comprises or consists of sequences from the gene CXCL2. In some embodiments, the sequences from gene CXCL2 comprise or consist of:

(SEQ ID NO: 896)
ATTCGGGGCAGAAAGAGAACATCCCACAGTTGGCGGGAGTTACGC
AAGACAGTCAGACCCGGACGTCACTCGTGAGTGCCCCGACCCCCC
TCCACCCCAGAGGCGGGGCCATCGCCTTCCTTCCGAACTCGGGAT
CGATCTGGAGCTCCGGGAATTTCCCTGGCCCGGGACTCCGGGCTT
TCCAGCCCCAACCATGCATAAAAGGGGTTCGCCGTTCTCGGAGAG
CCACAGAGCCCGGGCCACAGGCAGCTCCTTGCCAGCTCTCCTCCT
CGCACAGCCGCTCGAACCGCCTGCTGAGCCCCATGGCCCGCGCCA
CGCTCTCCGCCGCCCCCAGCAATCCCCGGCTCCTGCGGGTGGCGC
TGCTGCTCCTGCTCCTGGTGGCCGCCAGCCGGCGCGCAGCAGGTG
GGTCCCGGCGCCCTGGGGTCCCCGGGCCGGACGCGGCTGGGGTGG
GCGCCCCGCGCCGACAGCCCCGCTCAATCAGCGAGTCTCTTCTTC
CCTA

In some embodiments, the intronic domain that promotes trans-splicing primarily in the heart comprises or consists of sequences from the gene HSD17B13. In some embodiments, the sequences from gene HSD17B13 comprise or consist of:

(SEQ ID NO: 897)
GTAAGTACAGCACAGAACACCCAAATACTAAAACACCAATAGAGC
TTTTTTTTTTGCTTTTTTTTTTTTTAGACAGAGTCTCACTCTGTC
ACCCTGGCTGGATTGCGGTGGTTGCAGTGGCATGATCTTGGCTCA
CTGCAACCTCCGCCTCCTGGGTTCAAGCAATTCTCATGCCTCAGA
CCCCCAAGTAACTGGGATTATAGGTGTGTGCTGCCACACTACACC
CAGCTAATTTTTGTATTTTTTGATAGAGACAGGTTTCCCCATGTT
GGCCAGGCTGGACTCGAACTCCTGACCTCAAGTTATCCTCCTGTC
TCGGCCTCCCAAAGTGCTGGGATTACAGTCATGAGCCACCATGCC
TGGCCCAATAGAGCTATTATTATGGAGCATCTTTCAGTTGTGAAA
ATTGGCATGGAAACTCTCCATCCCTGGGGAGAACAGTTATTTCCT
CTGTTATTTTCCTACCCAGTCTATAAAAAGAGAGTGATTCATTTT
CTCT

In some embodiments, the intronic domain that promotes trans-splicing primarily in the heart comprises or consists of sequences from the gene HSD17B13. In some embodiments, the sequences from gene HSD17B13 comprise or consist of:

(SEQ ID NO: 898)
TTTACCCAGACCCTATTCAAGATGGAGTTGCTCTGATTTAAACGC
CTCTGACAAAATGACGACCTCAAAACAATCCAGCTTTATGGAATA
CCTCCACAAGAAAGAAAGTATACTTAGCTATAGAATTTTCTCCTT
GCATCCAACAGGCTTTGAGATGTCAGATGTTTCCTTCCTGTCCCA
TGATTAATCCTAGCCATTCCTCTTTCTTGTCTGGCTCCACTACTC
CTTACCATCTAATGCCTCGCCACCATTTTGATATTTTGACTAAGT
GAGCTATGAAACACACCTACTGGATATGAAAGTATAAGTTTCTGA
TAACAAAACATCAACATGGGATGTGGAGGAAGTGGGTAGGGTGGC
ATTAATGCAGCAAATCCTGGAATATTTTAAATCTTCATTCTAAAT
TTAGTAAAAATATAGGATAATTTTCCTGCCATCATTTACTTATAA
AATTAAAATTTTAGAAAATAAAAATAATATTTTCCTCTTTTTAAT
CACA

In some embodiments, the intronic domain that promotes trans-splicing primarily in the heart comprises or consists of sequences from the gene ADH4. In some embodiments, the sequences from gene ADH4 comprise or consist of:

(SEQ ID NO: 899)
GTCAGTTTTTTTTTTCTTCATAGCTTTAAATTCTTTTCCTAGTAG
TTCTGAAATTCTTAGAGTGAAGGTTATTGAAAAGAAATAAAAATA
ATTTATTCAACCATTGAATCCCTAGAGCATATCCAAATACTACAT
AAAAACTATTAATGACTTATAAATAATTTATATAAAAAGGTAATT
ATAATTTGCAAATTGAAATTTAATTTGCATTTGAATGAACCAACA
TGGAGGAACAAAAAATAAACATTGACCTTATTGAGATAATTTCTT
ATATAGAGTGGAAAATAATCTTAAATTTTTCTTATAAAGAAAAAT
TTTAAAATAGATATAAGTACAGAAAATTAAAGATGGAAGGAGTTT
AATCTATTTGAAGCAAACCAAATCTGACCTAATAATTTCATTCAT
TCAGCAAATGCTGATTAAACTACTTCGTGTCAAGTGCCATGTCTT
TATTTTGCATTTTGTCCACATTACAGGTTGGAAAAGTGTAGATTC
TATC

In some embodiments, the intronic domain that promotes trans-splicing primarily in the heart comprises or consists of sequences from the gene ADH4. In some embodiments, the sequences from gene ADH4 comprise or consist of:

(SEQ ID NO: 900)
TTTTTTTTTTTTTGAGACAGAGTCTCACTCTGTGACCCAGGCTGC
AGTGCAGTGGTGTGGTCTCGGCTCACTGCAACCTCCTCCTCCTCC
CGGGTTCAGGCAGTTCTCCAGTCTCAGCTTCCCGAGTAGCTGGGA
CTACAGGCAGGCACAACCACACCCAGCTAATTTTTGTATTTTTAG
TAGAGACGGGGTTTTGCCATGTTGGCCAGGCTGGTCTCGAACTCC
TGGCCTCAAGTGATCCACCCACCTTGGCCACCCAAAGTTCTAGGA
TTACAGGAATGAGTCACCTGCACCCGGACTGTGCTGAACATTCTA
TTTGTAATGGAACACTGGGCCCTCAAGGCTCAATCTACAATGATA
CTATGATGTCATTAGCTTGAATCCCATTATCTTACATGCAGAGTT
GGATTAATTTTTTTCTAAAGCATTGTACATTGCCACACTGGAGTT
CATATTCTACTTTTCCACTGACTCAAGTATTCTTTTATGCTCCAT
CACA

In some embodiments, the intronic domain that promotes trans-splicing primarily in the heart comprises or consists of sequences from the gene ADH6. In some embodiments, the sequences from gene ADH6 comprise or consist of:

(SEQ ID NO: 901)
TTTGAATGTCTTGTTATGGTGTAGCTCGTTGCAAAATTCACCTAA
TTTTATCACAAATGCAGGAACTTTTTTTCCTTCCTGTTAATATAC
AGTCAAACATATTACCAAGTACTGGAGACTATCTATATCACTGTA
TAACCATTCACTGTTGTTGCCAGCTTCAAGACCACATTTGAATCC
TATCAGGCCCAGTAAACTCTGTGAATAGACTCTGTGAACTGTCTC
TAAGTCTCACTGTTTTGTCCACATTAAGTGTCTTGTTTGAAGCAC
AAAGTAAAATCTTTCTTTCCAATCCCTGTCACTGCACCTCCCTAG
ACAGTCACTACATGGGGCAGTTTATGGACTTCAGCAATGTTTACC
TAACTCCAGATCCACACTATAGGAGTCCAGCCTTCAGGGAGAACT
AGTTTTTTACATTTTTTGCCTGCATAATGAGAAGCTTTAAGTTTT
TCATATTTTTACCTGAGAACAAGAATAATTACATTCTAGGCTATC
ACCC

In some embodiments, the intronic domain that promotes trans-splicing primarily in the heart comprises or consists of sequences from the gene ADH6. In some embodiments, the sequences from gene ADH6 comprise or consist of:

(SEQ ID NO: 902)
AAAATACATAATCCTGAAAGTATTTAAGTGTTCACCTACCGTTAC
TTTTGCCATTAGCATTGTATTTCCAATATGGATTTTTTTTTTACA
TGACATATTAATGGAAGTCAGCTTATAAAATTGTAGCTCAAGTTT
AAAAACATAAGAAGGTAGTTCACAAATAGGCCATTGTTGTATTTG
TTGTTCTACTTTGGGGGAGAAGAGGAATTATTCATAGGATTCCTA
ATTCTGAATCCTATCTTTGACCCAAAAAACAGAAACCAGCCATAA
CCCAGAAACTGGCATTCCCCACACGAGTATAAGCTTGCTTACCTA
CTTTTTACCCTTGGAGTTTCATGTCATTTTAAAAAATCTTCCCAT
GGCTTATTTAATCTTCTCATTTTTCACCTAAAGAGAAAAAAAATC
TAGTTTACTGCTCTACAAAGTGAGAATTTGAAATCCAAAACACAA
ATGTTGCTCTAAAAATGTTCCAACCTTTTTTCTCTTCTTCTTTTT
TTCA

In some embodiments, the intronic domain that promotes trans-splicing primarily in the heart comprises or consists of sequences from the gene ADH1A. In some embodiments, the sequences from gene ADH1A comprise or consist of:

(SEQ ID NO: 903)
TACTTGTGTCATCTTTGCCATTGAGAAAAGCTATTTCCTATGACT
TCCTGCATTTTTGGTATCTTCATAATCTTTAGTCATGGAATCCCA
GTGGAGGGGACCCTTCACTTGCCCTGAATATACACATGCTGGGCT
ATTGTGCTTGTAGTCTCCTGCATCTATCTCAATTTTCACTGTCGG
CATTTTCGCCTTTTTTTTTTTTTAATAAAATATACCGAATTCCTA
GGGTAAAAGCTGTAGTAGGGTAAAGGATATACTCATATTTACAAG
TGGTGAAAGTCCAAGAGTTGTAAATCCAGGAAATTTCTTAGGAAC
TCAAATAAAATGTCACATGTTTTCTTACAGTGAAGGAGAATGTTT
TTCTGACATTTATACTATGTCTTTATGGTCAATATACAATTGATT
TTTAAAAATAATAGCTGATTTCATGCTTCATATGACTAAGGTACA
ATTACTAATTGCAAAAACTGAACTATTCCTGGAATTATGTTCAAA
AAAT

In some embodiments, the intronic domain that promotes trans-splicing primarily in the heart comprises or consists of sequences from the gene ADH1A. In some embodiments, the sequences from gene ADH1A comprise or consist of:

(SEQ ID NO: 904)
ACTCTATACTAGATATGCGACCAGACCAAGTTACAAATCTTTCTA
AGTCTCAGTGTTTATAATCTCTAAAATCAAAATAATATGAAAATC
TACCTCCTAGTGTTGATGTTTAGGTTGAAGTTTAAGACAGAAAAT
GCATGAAGAATGTGACAAATAGCAAAAACTTATCACAAGTTATTA
TCCATTGTTGTTTTATATTCCTTATTATAACAGGAACGTGGGCAC
ACAACAATGATTTTTTGAAGATGCTAACGTAGTATTGAAGTCTAA
ATGACAGGCATACTCAGAATGTTGTGAGTGAGCTTAAAGTTGAAG
AAAACACAAGAGAAAACCAAAAAAGATGAGCTTTCCTTTGACTTC
ACTTGACCCGAATGCATGGGGGTGGGATTAGAGTGGCTGAGAACA
GACAGATTTTCACTAAGTCATTTCAAATGCTCCTTGGACTCTCAC
ATGGAAGCATGTCTAAATGCTAATACAATGTCTCTTCTTTCCTAT
TGCA

In some embodiments, the intronic domain that promotes trans-splicing primarily in the heart comprises or consists of sequences from the gene NDUFC1. In some embodiments, the sequences from gene NDUFC1 comprise or consist of:

(SEQ ID NO: 905)
GTAAGTGTTAAGAGATCTTTTCAACTGTAAAATATAATATGTCAC
CTTGGATGAGTAGCTGTCTTCTTTAAGCTTCATCTGTAAATTGAG
GCTTATAACCACGGCATGATATGTGCTCGTTTTGAAGATGAAATA
AGACAATGAATGTAAACCATATGATACATTAATGCACCGTGCCTG
GACCACAGAATTCAATCCAAATGCTAGTTATTATTCAGCACGACA
CTTTTCTTTCTTCTGGCTCATGTAGGAAAATTTTAATAAACCTCT
TGATGAATAGCCGTAGAGATGGGGTGAAAAACCCCCAGAGGACAG
AGAATAGGTAAACTCTTTTTTCTTTTAATTATTTTCTTCCTAATT
CCACTGTATAGACAGAAGGCTGAGGCCGGGGTGCTAGAGAACCTT
GCCGTGCTGGAATTCACGTTGACGCCCCCACGGAGCTCTGCTGCA
GAGCCCTCGAGTCCCGCACTTCCGGCCGCCAGGTGGCGCTGGTTC
TGTT

In some embodiments, the intronic domain that promotes trans-splicing primarily in the heart comprises or consists of sequences from the gene NDUFC1. In some embodiments, the sequences from gene NDUFC1 comprise or consist of:

(SEQ ID NO: 906)
TTTTGGTTTTAAAATTAATGACATAAAGTTTAAAGGATTAGTTTT
AAAGGGTCAAAATGTATTGTCTGTTACCTATAAATATTTTTACTG
GTCTTTAGACATCAGATTGAACTGAATTGAATGTACAAAGATCTG
GCTCAACTGTGCAAGATAAAACCTATTCCTCAGAACGATGTCTTT
AATAGGCAAAACCAAATAAATTGTTTACTACTACCAGACACTTGT
TTAAGCTTCTCAGCAACCCTATAGGTTTTATGTTACAGATGTAAA
ACTTAGGTATAGAGAGGTTAAGTAATTTGCCTAGAAATCATAGAG
CTAAAAAGTGGTGAGCAGAATGGCTTTGAACGCTGATTCTACAGC
TTATGGTCTAGGGTGGTCCAAGTCTGTTAGGTGGGCTAATGAGGA
TCACTAGATCTGGAGTCAGAAGACCTCAAACTGTATCCCACCTCT
GCCATCTGTGTGATCTTAAGCCTACCTAAGGCACTAATTCTACTT
CACA

In some embodiments, the intronic domain that promotes trans-splicing primarily in the heart comprises or consists of sequences from the gene SORBS2. In some embodiments, the sequences from gene SORBS2 comprise or consist of:

(SEQ ID NO: 907)
GTATAATATCACTAACTCACACAATGTCGCACTTGAATGGCTTCT
AGCTCTTCTGACTGTTTTTGTTCTAAACGTAAAAAGCAAAGGAAA
ATGAGATCTTGTAGGAAACATTTTAAATGTGTAATTAAAAACAGC
AGTCACTTGCCAAAAATAGGTTGAGTAACTGCGAGTGTTAATAAC
TATTCACGTTTTTGTTATCTCATGAACTTAAGTAGCATAATATTT
CATTAAGATAACTCAGAGGTTTTGGGGTTTTTTTTTTTAACCTAA
AAACTTTTGGGGTAGGGGTACAGGGGTACAGGATGAAAAAAGGGA
TTTGTAGGAAACTGAATCTTCATTAACTGGATTCTTTGTCAAAGG
TTGTGAACAGAATGATAGAACATTAGTTTTGAAAGAGACCTTTGC
TTTCCTTTAATCCAATCAGCAAATACCCTTCTACTATATATATTT
AAACATACCAAAATCTTGACCAATTAAGATTCACATGTACCCACA
GATA

In some embodiments, the intronic domain that promotes trans-splicing primarily in the heart comprises or consists of sequences from the gene SORBS2. In some embodiments, the sequences from gene SORBS2 comprise or consist of:

(SEQ ID NO: 908)
TTTACTGCAAGAATTATTCAAGCTCTCTTTGGGAATTATAACAGC
TAGAAGATGGTAAAACTTAAATGATATTAACTTAAAAGTATGTTG
TACCATAGCCTTTAATCCTGACATGGAGCTAATCCTTGACTCTAA
AAGCCAGATCCAGTGCTTATAGATTTAGTATAAATTGAATTTAAA
TGGTATAAACAAAGTTGAGTTCTGTGCCTGAGTTTTCACAAAGAA
AACCATAAAATGTTAAGTATTAAGTCTTCCATGCTTTCACTAAAA
CAGTGATACCTATTTCTAAGACAGATCCCTGGTCTTCTTTTGTTT
TGTTTCGTTTTGTTTGTTTTAGTTTGAGGGTGGTGTGAAATTGGC
AGGTGCATTCCATGATTACTAATTCTAACTGTTCATCTTTCTCAT
GGATGTCTGGCACATTTCTCTTAAAACGCATGATATTGCACGTGC
TTTTCCCGTTGCCTTGGCCTCTGCTATATCATTTTTTTGCTTCCA
GATA

In some embodiments, the intronic domain that promotes trans-splicing primarily in the heart comprises or consists of sequences from the gene KLKB1. In some embodiments, the sequences from gene KLKB1 comprise or consist of:

(SEQ ID NO: 909)
GATATGTACACAACTAAAACAAAACTAAGATATAAGATCATATTA
ATGTAATGATAATAAAAGTGGATTGTCTCCTGTTCTAATTTTAAT
AGGCACAAGGCATTTTTGTTAATCACTTCTTATTAAAGAATTTTT
TATAATATTCAGGAAAATACAACAAAAAAACCCTTACATTCCTAA
GGCCTCAGAGTCAGAGTAATATAAAGGAAATGTAAACACATGCTG
AGTAATGCAAGCATTTGGCAATGGTGGTGACTGGAGCTGGGAGCA
CAGCTTTTATTTCTCTGAAATAGGAATTTGCCCCTTAGAGTTAGA
CCAATTTTGCCTTCCTCTAAATGGCAAACAGTTTGGAGATACTTT
AAAGGACATTTTTTCACTTAGGATGTTTAGTACTATGAATAATAA
ATAGTCACAATTTCCTTAACTATGGTGACAAAATACAAGCAAATT
TAGCCTCATGTCATTTCCTAAGGAACATCTTCTCTCTGTGAGTTC
ACAG

In some embodiments, the intronic domain that promotes trans-splicing primarily in the heart comprises or consists of sequences from the gene KLKB1. In some embodiments, the sequences from gene KLKB1 comprise or consist of:

(SEQ ID NO: 910)
CGAGTATGCATGGAAAATCGCATCACAAAGGCGAGTATGCATGGG
GAGCACTTGCTGCTGTACTTTCATCACTTTTATAGTCTGAGTTCT
TAAAAGTTTCGTTCATTTCCCTCAAAACACTTGAACCTGCAGTTT
CAGTAGGTACTGTTCTGCCAGGTGCAGATTAGTTAAGAGATTAGC
AGACTTCTCTGCCTATCTTCTCTTACTTTAAAACAAATGTTACCA
TTGAATCAAGGAAGCAATAGCCATGAGAAAAAAGAAGGATCTGAC
GCCTTTGAATGAAGATTCAAAACATGATCTTCATGTTTTGTATTA
GCTTGGAGTAAAATCCACTTGCTGGCAATATAGCCCTTAAGCTTG
TTGCCTCTTCTCTTTGTTTCAGAAACTAGAGCCCTGTTTATTCTG
ATCAAGGCTCTGGCCCACTGTCTTTATCTCAGATAACCCACCCTC
TTCTGCACACAGCATGGAGCTAAGAGAAGGGTGTCTAGTTATGTA
ATAT

In some embodiments, the intronic domain that promotes trans-splicing primarily in the heart comprises or consists of sequences from the gene C6. In some embodiments, the sequences from gene C6 comprise or consist of:

(SEQ ID NO: 911)
GTAAGAGATACCCTACAGACTGTGTCTGGAAATTGGGAAAACCAG
TCTAGTGTAACCAATTTGATGGAAGAGCTGAGCTTTTTGAAGCAC
AGACATGAAATAAATGAATTTTCCACATAACTGATATTCCTGTGC
TCTGCTGAATTCCTCCTTCCCCATTGGGTAGCAAGTTTCTTAATT
ATCTCTCAGCCCAGTTCCCTGTTTAAAATGAGATTGTTCATTATA
ACAGTCTCATTTTGGTTTTAAAGACTTAACTTCTGCACATGTAAA
AACTTTAAACTCAGTTATATCAACACTTTCCTGTTCCTCTCACTC
AAGCCCAGGGCTTGGAGTGGGGTGGGGAAAACAGGCAAAAGAAAA
CTGGTTGGTGAGTGAACACACTACATTGGCTACGTGCCCCCTGTT
TTTTTCTGATCTTCATATAGAAATGCTTGCTTTCCTCTCTTGGGC
AGCCATCATTCAGAAGATCTCTGTGTGTTTGACACAGACTCCAAC
GATT

In some embodiments, the intronic domain that promotes trans-splicing primarily in the heart comprises or consists of sequences from the gene C6. In some embodiments, the sequences from gene C6 comprise or consist of:

(SEQ ID NO: 912)
AATCTTTCTAGCTACATCAATTTTTTATTATATGCTTCTCTTTGA
TAGCCCTAAGAAAGGCAACAATTCTATATTTATATTAGTGTGCTC
ATTTGATTAATGTCTGTCTGCACCCACAAGACTATAAGCTTGGTG
AGCACAGGATCTGGGTCAGTTTATGCTCAATCTACTCTCTCCAGA
ACCTTACAGAGTGATGAGAATATCATGTATACTCAATAAATATTT
GAAAAACAAATGAACAAAAATTTCAACAGTTCCATTTTCTTTAGT
TATCTGAACATAATCGCAATTTTAGGTTGGAAAATGACACCTCAT
GTGCACTGGAAAGGACCTTTCAGGTGGAATATTTAATGACAATGT
ATTTAGAGAAGCCAAAATGAATGATCTAACTCCAAGAGATAAATA
TGAATCCTTTACCACTGCCTCTTCTCTGAATTCAAGCTTATCACT
TAGAATCCATGCTGTGCACTGTTCTTTTCTCCTTTTTTTTTTGTT
TCA

In some embodiments, the intronic domain that promotes trans-splicing primarily in the heart comprises or consists of sequences from the gene MRPS27. In some embodiments, the sequences from gene MRPS27 comprise or consist of:

(SEQ ID NO: 913)
GCAAGTGCCCATTCCAGAGACTCACTCACCTGACATATTACTACT
GGAAGTTGAAAAGTCCCCACATTTATTTGTCTGAACTGCTTAAAA
AGTCACCAACAGGAATTCTTCTCAAACTTGCCATCCAAAATTGAA
CCCCGCTGGCCCAGTGTTTTGACTTGTTTATATTCCAGATAGCCA
CTCACGTAAGTTTATCTTTTTAATGCTGATGGGGGCCAAGAAAGG
AGTTCATTTGCTTCCTGGCTGGGAAATTCAGGAATTGCATTTTGG
GCAGCATATTTTATAGTATGTGTGTAAGTATTTCACTGCTTTTCC
TCAGTTTATTTTTTGTGAGAGTTCATTGAAAATGTCCAAGGCTGG
GCACAGTGGCTCATGCCTATAATCCCAGCTCTTTAGGAAGCCGAG
GTGGGAGGATCACTTGAGGCCAGGAGTTTGAGACCAGCCTGAACA
ACATAGCAAGACCCTGTCTCTACAGAAATAAAGAAGGAGGCAAAG
GCAG

In some embodiments, the intronic domain that promotes trans-splicing primarily in the heart comprises or consists of sequences from the gene MRPS27. In some embodiments, the sequences from gene MRPS27 comprise or consist of:

(SEQ ID NO: 914)
CTAGGCAAGCTCTGTCTTCACCTGAAAATCCTACTCAACACTGGG
GACAGAGATCTCTTTAGCCATGGGATACCAACTTTTAGAACTAGA
GCTAGAACTTGAGAGTACACTTTGTCACTCTTTCTTATGCTCCTC
CAAGAAATACACATTTCCTCAGAATTGTAAGTGTAATCACATTTT
ATAAGCTGAAATCTCAGAGTACCTTTAGCCTTCATGAGCACAACC
TTGTTGGGGATATTTGGGTTGTATTTGTTTCTCTTTTCTACACTC
ACATGTTTGCCTATCTTGACAGCCTGTTGGCCTAGGCAATAATAA
AGCTTCTGTCGAGGTGAGCAAACAGCTGATGGCATAGGCGCACAG
AGGGGACTCCTACAGTGGCTGGCTGTAATCTGCCAGGCTGGTGGT
CAGAGCAGGCGAGAACAAAAGCCAGTCTGTTTGCAGGCAAATTGG
AAAATCAATGTGTTAGTTGTATTCTTTTCTCTTTGCCTTTCTTCT
GACA

In some embodiments, the intronic domain that promotes trans-splicing primarily in the heart comprises or consists of sequences from the gene DMGDH. In some embodiments, the sequences from gene DMGDH comprise or consist of:

(SEQ ID NO: 915)
GTAAGCATATCATTTTTTTGAAATTAGTGTTTTTTAAGTGCCAAA
GAAAGAAGATATTTAGCCGTAATGTTTCGTACGATGGACATTGTC
ATGAAGCACTGTGATGCTCCCAAGTTAAACATTTTGTCTGTTTCT
AAGTGACAAAGCCCTCCCTCTATCACTTTGCTCTTGGTGGATGCG
CTATCACTCTGTCCTTGGTTATCCAAAAAAAGTTGCTTATTTTCT
CCATTCTGCATTTTATTTTGCTCCAGATTGTGTTTTTCCAAATTA
TGTTTATTTTTCCTTCCTGTATTCACTGTTTCTCTATATGAATAA
CCAGATTCTAGTGTTGAAGTTCATAAGCATCCTCTTTGTGTCCAA
TTTGCTTTTTAGGGCAGATTTGATCATTTGGGTTCTCTCTCATCC
ATTTCTGATGACTTTTGACTCTAGTTGAAAAAGTTTTTAAATGCT
TTTCCTTCTAGAACATTATTAAACATATTTCATAATCTTCTCTTT
TTGT

In some embodiments, the intronic domain that promotes trans-splicing primarily in the heart comprises or consists of sequences from the gene DMGDH. In some embodiments, the sequences from gene DMGDH comprise or consist of:

(SEQ ID NO: 916)
AATAGATGAGACCCATATATAATATTTCACTGATCATTCTATCAA
CGTATCCCATCTGAGTTTCATTCTTCTTTGTTTTCCCCACAGTAC
CTTGCCTGAAGGCCTAAAACAAAGTAGATATGCAAGAATTAATTG
TTGAATGATTGAATCAAACATAAATTTCCTGACACCAGATATATT
ATTTAATGTGAATTTATTAACAGTATTAAAAATTAAATATAAATT
ATAATTGATATATCAGTTGGAATTAGATTTGTCTTCAAATGTCAG
AAACCAAAATAAGAAGGTGTAAACAAGATAGAGGTTTATTTCTCT
TTCTTTTCAGCAGTCCAGGACTTGAATGGTGGCTTTTTCATGAAG
TCTTCAGGGACCCAGTCACTTTCCAGATCAAGAAACACTTTTGAA
GTCCAGTTTTACTAAACGTTTACTAGCAGGATATATTTTTACTGA
GTAATAAGTATATATATATTTGAGTATTGACTCTGGTATGATTGA
TTTA

In some embodiments, the intronic domain that promotes trans-splicing primarily in the heart comprises or consists of sequences from the gene ZCCHC9. In some embodiments, the sequences from gene ZCCHC9 comprise or consist of:

(SEQ ID NO: 917)
GAATGCTCAACGAGCACTGAAGGGGGGACATGAGGCACTGCCTCT
CACCCAAGAGAGCCCCCAAATGTCCTTTTCAGACTTTTATCACAG
TGACCCACTCCCACCGCGCCTTTACCTTGCCTCTGCCTGGAATGT
CCTTAACTCCCTCTTGCCGGCAATTTCACCAGATTTTTGATTTCT
TATCCAGTCTCACGGGAAGCCTTTCCGGCATCTCCCACCCTACCG
ATCTTCCCGTCTATCTCGCCTGCTACACTGTGAGTTCAGACAGCA
CGGATTGAACTCTTCATCTTTTCGTGTCGGCGTCCTGCACAGGGC
CATCCCATCACTAAGCGCGCCACAAACTCAGCCCAGTATTGACGC
CCACGAGTCGCTTGTAAAGACCGTCTGTGACGCCGGAAGTGACGC
GCTCTCGGGCACTATGTGCTTCCGGTCTCCAATACCGCAGGAGGG
CGGTCTTCCCCGGCTCGCCAACTCGGCTGCTCTGGGGGATTCGTG
CGCG

In some embodiments, the intronic domain that promotes trans-splicing primarily in the heart comprises or consists of sequences from the gene ZCCHC9. In some embodiments, the sequences from gene ZCCHC9 comprise or consist of:

(SEQ ID NO: 918)
TGAGGTTCTCAGCCACCAAAGCTGCAGGGTCTCGTCGTTCGTTCC
CTCTGGCCTGGCGGAACTTGCGTGGGAAGCTGGGAGGACAGAAGG
CTTAGCCGCTGGGTCCCTGAGCTGGCTGGAGCGCGGGCTGTCTAG
GGGCGGCGACCTCAGCTGTTGGGCTCCGGCTGCTGGGGGCTGTGC
GTCCAGCCGCCCTGGAATCCGCCTGTGGGTGGGCCGCTTGGCTCT
CCCACTTCACCCTCTTGATAACCCAGAGCCTCCCCCTTCCCACGT
ACGTAGGGGAAGGATAATCTACTTCCTTTTGAACAGCTTCGGTCC
CCACTATGACAGTCGCATAAACTTCAGCGAAGAGTGCGCTGGTAA
AGTGGCATGGAGTTTCTCACACCAATTCATTGGGTCCACCCTTCA
GCTTCTGTGGAAGGGGTCTTTCTGTTACTAGGAGCTGAGACTTGG
GGGGTTGCCAGGGATGCTGGTCTTAAAAGGTGATAATTTAGGCCG
GGCG

In some embodiments, the intronic domain that promotes trans-splicing primarily in the heart comprises or consists of sequences from the gene LEAP2. In some embodiments, the sequences from gene LEAP2 comprise or consist of:

(SEQ ID NO: 919)
GACGATCTGGGGAACTGTAGGCCTTGCTGAAGCATCCTCCCACAG
AAAGGAGATAAGGGCCTTATCCAATTGGCTGTACACAATAGATCA
GTGCTTCTTTCCTAGACAAGGCTGAAAGGGGCCAACATTATTTCT
GAAGACTTCATTATTGGAATTCTATGGGAGTGATCTCACTGAGCT
ATTTTGGAATAGAAATGTGGCTAGTTGCCTGACCTCCCTCAATGG
TTTCACGTGGCTTTCAAAGGGAAGGAAGGGCAGTGCTGACTTTTG
GTAAAATGGGCGAAAGGGTCCATGCCAGCAACACAATCACTCAAA
GTCCAGATGAGGGATCAGTAAATACAACGTGCCTGAAAGGTGGCC
CTTGAGCACATTCCTCCGGTAGACATTAACTTATTAAATTGATTC
TGATTACAAATATAAACTTTGCCCCCATCTCACCCAGTAACAATG
CAAGAGTTGATGTCAGTCTATAAAAGGAAGTAGGAACTGTCCCTG
GCTT

In some embodiments, the intronic domain that promotes trans-splicing primarily in the heart comprises or consists of sequences from the gene LEAP2. In some embodiments, the sequences from gene LEAP2 comprise or consist of:

(SEQ ID NO: 920)
TAAGGAGGGAAGGATACTTATGTGTGTGTGTGGAGTGTGGAGATG
ATAGTGGTGGTGGAACTTGAAAGCTAGATTCAGTCCTGAGGAATG
GTTCCTCTGTTCTGAGTCTACAGCATCTGCGGAATGGAATGATCA
CTCTTCCAAGGTGTGCAGCAGGGTGTCAACACTTTCATATCTGAA
TGTCTTTGCCCTTACAGATAGATGGCTCCCCAATACCAGAAGTGA
GTTCGGCAAAGAGAAGGCCACGGAGAATGACCCCATTTTGGAGAG
GGGTTTCCCTCAGGCCTATTGGAGCCTCCTGCCGGGATGATTCTG
AGTGTATCACAAGGCTATGCAGGTACTCCCTGAACCTGGGAGCAG
GGTTGGGCCAGAGAGCCCTGGGAAGCTGGGCAGAGGAGTGACAAG
GGGACACATGAACCTAAGAATAAAGCTGGGATGGAGGAGTTCTAG
ACTGAGACTGGGAGCTCCATGGAAAATCCCTTAGGAGTTAGGAGC
CAAG

In some embodiments, the intronic domain that promotes trans-splicing primarily in the heart comprises or consists of sequences from the gene ETF1. In some embodiments, the sequences from gene ETF1 comprise or consist of:

(SEQ ID NO: 921)
GTAAGGGGCCCGCGCTCCCCGGTTTAGATCACTCGGCCCCCCTCG
TGGTAGGCCACCGCTACTTTTGCACCTCTCCCGGGCCCTTAAGTC
CAGGTCCTCCCTCTTCGGGCTCCAGGTGCTGCCACCCGACTCGGG
TGCTATTGTGGCAAAGTGTTCGGCCTTCCTTTCCCAGGGCCCTTC
CCTGATTTCCTCTTCTCCCTTCATTTCTCAGCCAGGCCTCATTAA
AATGGCCTCTCCCTTCTGGGCTTCTGAGACCCGGGTGGGTGAAGT
TTGGATGGGACGGGAAGCAGAGGTGGCCAGGGACAGCTTCTTTCT
GATTCCTGATTTCTCTGGGTCTTCCACCTGTCCCAAGATGACTTC
TCCCAAGTCCTCTAATTACCCCGGAGCTTCCTGGTTTTAGCTGTT
CCAGGACCCACCTATAAACTGAAAATGCCAACGTACGCGCGATGT
GAGGGGTGCTGGGTAGGTTTTGGTTAATGAATCTGCATGTGTATG
TCCA

In some embodiments, the intronic domain that promotes trans-splicing primarily in the heart comprises or consists of sequences from the gene ETF1. In some embodiments, the sequences from gene ETF1 comprise or consist of:

(SEQ ID NO: 922)
GAGAAGTCGCGGCGGACGCCCAGCTTCTCCTCACTCGCACCGGCC
CCCTCCCTTCCGCCTGCCGCCTTCCACTCCGCCCCTGGCGGAGGA
AGTGATGTCACAGGCCCCATGTGAGCGGATTGCAACACATGCAGC
TGCCTGGAGAGAGGGAGCCGGTGTCCTACGTCAGAGCCGCCGCCG
CCGCGGAGCCGCCGCCGGGGAGGAGCAGCCGCTGCCGCCCAGGAC
TGGGCCCTTAGGTAAGGCACAGGGAGGGAAAGGCCGGTGACCTTT
GTGGCGAGCTGCACCGGACGGGAGGAGAGGGCCTCTGGGGGATGG
CGAGGAAGGGGGCCGCTCTGCCCCAGCGGCGGCGCTAACTCTGTG
GTCTTGGTGTCTCCGGGCCCCGCCGGGCCGCTCTAGGGAGGAGGA
GGCGAGAAGATGGCGGACGACCCCAGTGCTGCCGACAGGAACGTG
GAGATCTGGAAGATCAAGAAGCTCATTAAGAGCTTGGAGGCGGCC
CGCG

In some embodiments, the intronic domain that promotes trans-splicing primarily in the heart comprises or consists of sequences from the gene RNF14. In some embodiments, the sequences from gene RNF14 comprise or consist of:

(SEQ ID NO: 923)
CATTGCTGTCTTTTATTACATCTTTTTATATTAAAAATAAATAAA
AATGTCATAAATAGTAATTGTGTAAATATAAAAGGCCTTATTAAA
AAAACAAATCTTAGTGCCTCAGTTTTCTCTTCTGTCAAGTGGGCA
CATAAAATTACCTAAGTCATAGGGTCCTGGGCATTTAAATGAGAG
AATGTACGTGAAATATTTAGCACAGTGGAGGGCTAGGCACACGGG
GAGACTGCAGTTTAAAAGACTAACCAGGCACATGAGACCAAAGAC
ACAAAAGTCTGTTATTTTCCTAGAGTCAAAAACAAAACTTATTAG
AAGGCTGTCTTTACCCAATCTTGTCCTCAAAATTGACTCTAAATC
TTTAGTTCACTTTAAACATTCAGTAAATTATTAACACTGCTTTGA
AATTCTCTTTCTATTCTCTCTATCCTGTGACCTCTGACATGATGT
CACAATTCACCTTCTGCTCTGACTTTGTGAGGATTTCCCAAGCTG
GTTG

In some embodiments, the intronic domain that promotes trans-splicing primarily in the heart comprises or consists of sequences from the gene RNF14. In some embodiments, the sequences from gene RNF14 comprise or consist of:

(SEQ ID NO: 924)
TAGGGTTGCTGTGTGTTTTGCTCTTGGTCTAATTCCTTTGGCTAA
ACATGAATAATTATATTAATGAATGGCTTTGGAGATTCTTCTTCC
GTCCTTTGCATTTTTCCGAGATATGTTTCTGGTTTTGGTATCTAT
TAGAAGGGTATTTTGGTTTCTGTTTTGTGACAATCAAGATTTATA
GTACAGAAAATTAGAAGTGATTATTTCTATTTCAAGGTTCTTTCT
GTGGCAGCCAGGTAATCCCTTGGGTGCCCTGATTTACTCACTTGG
TTTTTTTCTCTCTCCTGGGTATAAGACCAACTACATAATTTACAA
AGCCCAGGGCAAAATAAAAATGCAGCCTCTCATTCAGAAATTATT
AAGAACTTCAAGAGGGTGAGAGCACAGCATCAAATCAAGAGCAGG
GCACTTCTTTTTTATTTTAATTTTTCCATAAGTTATTGGGGTACA
GGTGGTATTTGATTACATGAGTAAGTTCTTTAATGGTGATTTGTG
AGAT

In some embodiments, the intronic domain that promotes trans-splicing primarily in the heart comprises or consists of sequences from the gene NR3C1. In some embodiments, the sequences from gene NR3C1 comprise or consist of:

(SEQ ID NO: 925)
GTAGGTTATATTTTTATTCCCATTCTATAGCTGGGTAAAGTGAGT
TCTAACAAAGTTACTTGTTAAAGGTCACTCAGAGGTCAGAGCATC
AGAAAAAAAGACAATCACAAGGCTGATGTTGTGTGCTGGATAGTT
TAAACTGAACAGGAAGAAAACATTTTTGTGGGCTTTATCTAAAAA
GAAATAGTTTGCTCTGAGTTTCTCAGTTTCATTTATTCAGCAAGT
ATGTGCCAAGTGCTATTGTAGGCAATGAAGATACAGCAGGGAACA
AAACAAAACTGACATGGAGCTTCCAACTAGAAAGGAGAACGAGAC
AACGCGTTTTAAAATATATATAATGTGTTAGGTAAAAAGTGCTAT
AAGGAGAGCTAAAACACAATGAGAGGCTAGAAGAGTGATAGTGGC
GGAGGGGCTTCTCTGAGGAGATGACATTTGATCAGGGGTCTGAAT
AAAGTAAAGAGGCCAGTTATGTGAATATCTGGAAGACTCAAGGGG
CAGG

In some embodiments, the intronic domain that promotes trans-splicing primarily in the heart comprises or consists of sequences from the gene NR3C1. In some embodiments, the sequences from gene NR3C1 comprise or consist of:

(SEQ ID NO: 926)
TAACTATAAATTGTGCATTCTTGCATAGATCCTCCTTGGGAATGA
GAAATTAGGAAAATCCAGTTGTTAAAATGAATGCCTAAAATCAAA
ATAAAATTTGTTTTTCTGGCACCTGCTTGATGACACAGACTAATA
ACCAATGACAAAATTGCCCTTGAACCCAAGTTTTCATTTCCTCCT
ATTGTGTGGTCAGGTTATGTAAGGGTTTGCTTTCACCCCATTCAA
AAGGTACCTCTTCCTCTTCTCTTGCTCCCTCTCGCCCTCATTCTT
GTGCCTATGCAGACATTTGAGTAGAGGCGAATCACTTTCACTTCT
GCTGGGGAAATTGCAACACGCTTCTTTAAATGGCAGAGAGAAGGA
GAAAACTTAGATCTTCTGATACCAAATCACTGGACCTTAGAAGGT
CAGAAATCTTTCAAGCCCTGCAGGACCGTAAAATGCGCATGTGTC
CAACGGAAGCACTGGGGCATGAGTGGGGAAGGAATAGAAACAGAA
AGAG

In some embodiments, the intronic domain that promotes trans-splicing primarily in the heart comprises or consists of sequences from the gene ECI2. In some embodiments, the sequences from gene ECI2 comprise or consist of:

(SEQ ID NO: 927)
GTAGTGCTAAAAAGTGACGACCTCACGCGCGCCCTCCTGCACCGC
TGGGCCCGGAGTGGTCAGGATGGGCGCGTCGGGAGGTCAGGGCTA
TGGTTCCCTGTCAGCAGTCCCCAAGGTCTCCGCCTGCCTCCGCGC
ACGCTCCCATCCTCCTGGGGCCGGACTCCGGGCGGGTGAGCCGCC
CCAGCTGAGCACCCGGGCCTGCACTTCCCAGCCGGGCTGCTTCTG
GGGTGCAAAGCGTCGCTTGCTAAAAGCGAGGTAAACGCAAGCCGT
TATCACTCGTGTGGACTTAACACTCTGCTTCTTGGAGAACACGTA
TGCTATAAAGTGAAATGGCCGGGTACAGAGTAACGGCTAAGAAGC
ACGCTGCACCTCCCTGGGGTCCCGGGAACCCGCACGAATCTGTGC
ATAGGGAACAAAACCGAAACACGGTGGTGGCCTTGAAGTGGTAGA
ATTATGAGTGATTTTCCCGACTTTATGTATTTTCTTAAATGGTCG
TGTT

In some embodiments, the intronic domain that promotes trans-splicing primarily in the heart comprises or consists of sequences from the gene ECI2. In some embodiments, the sequences from gene ECI2 comprise or consist of:

(SEQ ID NO: 928)
GGCCCCGCCCCCGCATCTGAGCCCCGACTCCTAACCGCCTAGAGC
TCCCGCCCCGCCCACGCACCGACCCCCCCGGCCCCACCCCCGAGC
CCCCGCAGCCCCAGAGGAGGCCACGGGCTAGCGATGGCGAATGCG
GCGTGGCTACGCTCTCAGCCTGGCCCCGCCCCAACCCTGGCCCCG
CCCATGCCCCAGGCTCCTGCCTGGGCTCTGCTCTCGGCCCCGCCC
CGCACCCAAGCCCCGGCCCCACCCCCGAGCCCCCGCAGCCCGGGA
GCCTAGGCCCCGCCCCCGAGTCCCGGCTCCACCCCCGAGACCCGG
CCCCACCCCCGAGCCCCCGCAGACCGAGCTAGGCCCCGCCCCCGA
GACCCGGCCCCACCCCCGAGCCCCCGCAGCCCTAGAGCCGCCCAA
GGGATGGCGATGGCGTACTTGGCTTGGAGACTGGCGCGGCGTTCG
TGTCCGAGGTAAGCTTGGAGTCCCGGCCGTTCGGCCCATGGTCGC
CCGC

In some embodiments, the intronic domain that promotes trans-splicing primarily in the heart comprises or consists of sequences from the gene ALDH5A1. In some embodiments, the sequences from gene ALDH5A1 comprise or consist of:

(SEQ ID NO: 929)
TGTTCTTTGTGCTTCTTGTATTTGGATGTCTAGGTCTCTAGCAAG
GCTGGAGAAGTTTTCCTCGATTATTCCCCCAAATATGTTTTCCAA
ACTTTTAGAATTCTCTTCTTCCTCAGGAACACGTATTATTCTTAG
GTTTGGTTGTTTAACATAATCCCAGACTTCTTGGAAGCTTTGTTC
ATATTTTCTTATGCTTTTTTCTTTGTCTTTGTTGATTGGGTTAAT
TTGAAAACCTTGTCTTTGAGCTTTGACGTTCTTTCTTCTGCTTGT
TCAATTCTATTGCTGAGGCTTTCCAGAACATTTTGCATTTCTATA
AGTGCACCCATTGTTTCCTGGAGTTTTGATTGTTTTTTATTTATG
CTATCTATTTCATTGAAAACTTCTCCCCTTATTTCTTGTATCTTT
TTTTTTTTTTTTGTGACTTCCTTAAATTGGGCTTCGCCTTTCTTT
GTTGCCTCCCTGATTAGCTTAATAAATAACTATCTGAATTCTTTT
TCAG

In some embodiments, the intronic domain that promotes trans-splicing primarily in the heart comprises or consists of sequences from the gene ALDH5A1. In some embodiments, the sequences from gene ALDH5A1 comprise or consist of:

(SEQ ID NO: 930)
TGAGCTCATGTGATTTTTGGGGGGTGTTAAAGAATCTTGTTTTTT
CATATTATCAGAGTTGGCTTTCTGGTTTCTTCTTATTGGGGTAGG
CTCTGTCAGAGGGAAGGTCTAGGGCTGAAGGCTGTTGTTCAGATT
CTTTTGTCCCACGGGATGTTCCCTTGATGTAGTAGCTCCCCCTTT
TCCTAGGGATGTGGCTTCCTGAGAGCCGAGCTGTAGTGATTGTTA
TCTGTCTTCTGGATCTAGCCACCCAGCAAGTCTACCAGGCTCCAG
GCTGCTACTGAGGGTTGTCCGCACAGAGTCCTGTGATGTGAACCA
TCTGTGGGTCTCTCAGCCGTAGATACCAGCACAGTATTTGGGGTG
TCTCCCAGGTCCTGCAGGAGCAATCTGCTTCCTTCACAGGATCTG
TATGCTCTCCCAGTTTTCCTGATTTATTCCTGCAGTCGTTCTGGA
GCAAAAGTTCACAATGCAAGCCTCCACATGCTGTTTTGTCCATCC
GAGT

In some embodiments, the intronic domain that promotes trans-splicing primarily in the heart comprises or consists of sequences from the gene APOM. In some embodiments, the sequences from gene APOM comprise or consist of:

(SEQ ID NO: 931)
CAATCCACCGTGCCCCAGCACCTGCATCATACCTGTTCTAAAGCA
TTTACCAAGTTGTATTGCAATGGTTTGTCTACACAGCTAGTTTTC
CCTCTAACGACTTCTTCAAGATCAGGGGCTATGTCTTATTCGTTT
TTTTATGTCCCCGGGGATTAGCTAGTTCTTGGGAAACAACTGGGA
CTTGGGATTCAAAACAGTTTGCTAAGTGAATGAATGAGAGGCCCA
GTCAAGCTACTTCCCTTCAGTGCTGCACAGTGCAACAAATAACCA
GCTCAGATACAGGTTCAAAGACCACAGGCTTCTCCCTGGACAGCT
CAGCTCTCCTCATAACTCCTGAGAAACCCTGGACATGCCAGGGTG
TACTATGGAGTGGTTGCATCCGGAAGAGGGGGAGGAAGTCCCAAA
CACTAAGTAATCCAGGTTTGGGTTGGAAAACAAGGTTGAAGTTAC
TCATTAGCAGGTGAAAGGGTCAAGGGTCGAACGCAAGGGAGCTGA
AAGC

In some embodiments, the intronic domain that promotes trans-splicing primarily in the heart comprises or consists of sequences from the gene APOM. In some embodiments, the sequences from gene APOM comprise or consist of:

(SEQ ID NO: 932)
TATGGACTGAGATTGGGGGAAGCCTATGGTGGAGGCTCTGAGGGA
CTTGGGTGGATGGCCTAGGATGACTGGAGACCATCTTGGGAAAGG
AAGAGAGGAAGGGGGTGTGAGTGTTGTGATAATGAAAGCAAGAAG
AAAAATATCAGTACTGTGGCCATCAATGCAGAGGCATGGCAGAAT
TGGGGGGTGGGGTGGTTACCCAGGTTGACTGGGGAGGGGCAAAGA
GGAAAAGTCATTTAATGACTCTTTGTCATGGATCCAATCCCCAGT
TGGAAAGAGGAAGGCAGCCAACACCTCTACCCCTAAATCTTGCTG
TTTTGACTGATGAAGAGGTTGAACCCATCCTGTGCTGGAACCCAC
CCTCTTTTGCTCCCTTCATTGTCTCTCCAGTTCCCAGAGGTCCAC
TTGGGCCAGTGGTACTTTATCGCAGGGGCAGCTCCCACCAAGGAG
GAGTTGGCAACTTTTGACCCTGTGGACAACATTGTCTTCAATATG
GCTG

In some embodiments, the intronic domain that promotes trans-splicing primarily in the heart comprises or consists of sequences from the gene FKBP5. In some embodiments, the sequences from gene FKBP5 comprise or consist of:

(SEQ ID NO: 933)
GTAGGGACCCCATGGAGTACGTGACGCCTGGCCCTCCAGCTGCAT
GCTCCAGTTATCAGGACAGAGACCCCTCCCCACCCATTCCCAGGC
TTCCCTGGCTCCCTGCTCTGCTGCTGGGAGGCCCTGTCACCACAC
ATGCGCGTGCACGCACTAACATACACACACCCCTCTCAGGCACCC
CCGGAAGACCAGCTGCAGCTGGGGGTGGGGGTGCACGGAGCACAG
CCAGGGTTCTCAGATCCCTAGCCTGCTCTCCCACCCCGCCCTAGC
CCCCAACTGCCGAGCTGCTTCTAGTTTTAAAGCAAAACATGACTC
TGTCCTTCCTGCTGCTCAGTGGCAGCAACACTGCCTGGGGTTATT
TTTAGCAGGGATGCAGGGAAGGCACAGTTCTCCTTGACTTTAACT
AGGGACAACCCCCACCCCTCAGCCAAGACTCACTGGAGGAGGCCG
GGTACGGAAACCCCCCTCCCAGAAAGGGGAAATGGTGTGTTTTCT
GTTC

In some embodiments, the intronic domain that promotes trans-splicing primarily in the heart comprises or consists of sequences from the gene FKBP5. In some embodiments, the sequences from gene FKBP5 comprise or consist of:

(SEQ ID NO: 934)
GGACAGTCATCTTGGACATGCTCTGTGTTCCATATCATGACTTCA
AGCCTTGCCTTTTTCATGTACTACCAGTGGGATCCTAGACTAGCA
CTTTACCACTTTAAGCCTCAGTTTCCTAATTTGTAAATAAGGAGT
AATTGTAATCATTCCAATATCACAGGCTTGCTGGGAGGATTAAAT
GAGATCATCCACATCAGGGTCAGCACAGTGCCCGGAATGGGGTAC
CTGTGAAATTAATGGTGATTCTTCTTTATTGTTACCTTGGAAAAT
GGAGGCCAACTGCAGGATGATCGTGGCTCCAAGGAGCTGGGGACC
CAAGACATGCACAGAGAAAGAGAGACCAACAATGTGAGGCCTCTG
ATGCATGAGACAGGCAGGGGAGTAGTGGGAGGAGAGAGCAAGAGT
GAGCAGCCAGGAGGGCCTCTTCCCTTCCCTTTACTCAGCCTGTCA
CTCCTCTGCTAGGATCCTGCATTGACACATCTTGTACTTCCTGTT
TCCA

In some embodiments, the intronic domain that promotes trans-splicing primarily in the heart comprises or consists of sequences from the gene SLC22A7. In some embodiments, the sequences from gene SLC22A7 comprise or consist of:

(SEQ ID NO: 935)
AGGTGTGTCTTAGGCCAGGGCATCAGCTCCCAGCAGGGGAGAGGG
ATGCGAGGAGGCCTCATGGCTCAGCAGAAAGAAACCTGGTTAGGA
TCCAGCTTTCAGTCCTGAATCCTGGGCTGTCGGCCAGGCCATGGC
TGTAGCTGCAAATAGGCAAGGCTGACCTAAGGTCTCACAGGACAA
AGGGCACCCTGGGAGAGCTGGCCCCAGGCCCCAGGGGCCTCCTCT
TCTTAGGGATCCTGGGAGCTCTGCTGACCAAGGGGCTAGGGGCTC
ACCTCCTATGCCATCACCCAGCTACCTCATCATGGTAGCCTTGGT
CCCTGAACACTAATGGGTAAGCCCCAGAGCCACGAGCCAGTGACC
TCTGAATGGATGGCTTCCCCTTCCCCCAGGCACCAGGCAAGACAG
GTAGAGGTCCCTGGAGACCTGATGGCTGGGGAGGGCCCAGTCCTG
GGCCAGCCCCTGAGGGCTGGATTCTGGCTGCAGGCTCTCCAGTCC
ATCC

In some embodiments, the intronic domain that promotes trans-splicing primarily in the heart comprises or consists of sequences from the gene SLC22A7. In some embodiments, the sequences from gene SLC22A7 comprise or consist of:

(SEQ ID NO: 936)
TACTTAAGCCCAGAAGTTGAGAGACATGTGAGAGGGATGGGCCTG
CCATTGCCTTGGGTGGTTACTGTGTAGGCATTAGATGTATTACTT
TACCTCTTAAAGTCTCAGTTTACCAATCTGTAGATGGGGGCTATG
AATATAAGGCACTTTTAGTTCATGGCTTGTAGTAACAGGATCCTA
CACAAGAACTGTTATCATCATCATTAATTGGGAGGTGATTAAGGC
AGGGAAGGGCTAAAGTGGCTTCAGGGAGTTGAGACAAAGAGCTGA
GAAGGCAATTTCTGCAGCAAGAGGTGGAGAAACAATAGAGGCCTT
CTTTTCTCCCTTCCTCTTTTCCAGTCCCAGGTCGGTATTTACATA
ATCCATCTGGAGGTGGAATGTCGGTGGAGGCAGTCTCCCTGGGAG
GCAGCAGGTCGAGGCCTTCCTTGGGAAGAAGCTGAGGCTGCAGGA
CTGGGGAGGGACAAAGTTTCCTATTCCCCAAGCTGGCGTGAATCG
TTGG

In some embodiments, the intronic domain that promotes trans-splicing primarily in the heart comprises or consists of sequences from the gene MLIP. In some embodiments, the sequences from gene MLIP comprise or consist of:

(SEQ ID NO: 937)
TCACTACAGATGCTCCTGTATTGTTGCTTACAATTTATATACTAA
GGATAAAGTTACTGATATTTTGAAATAACTGTGTGTTTTCTAGTT
CTTTATTTGGACACAGTGTTTGCATGTATTTATACCTCTAAATGT
TCTCTATGAGCATGACCAACTGATTATTGAGATTGGCAGGTGCTA
GCGCCCTTCAATCAGCTTCTCATTGCTGCACAATCTGGCTTTTCT
CTTCTTTTCACAGAATATATAATTGGAATACATGCATGAAAAGAA
AAGAGGGAAAACACATATCAGTGGCTTTTTTTCTTTTTCTTTTTC
TTTTTTTCACTTCTACTGATTCCATTTCAGCAGTTAGTATGACCT
CTCATAATGTTCCAAATCTAAAAATTGTCAAAACAGAAATCTACT
CTTCGGAGCTGACACAATTCCTCAATTTTGGAATGACTCTGCTTT
ACCTCTTTTTAGAATTTCTTCTTTCTGCGTGAGTGTGTGTGTGTT
GTTT

In some embodiments, the intronic domain that promotes trans-splicing primarily in the heart comprises or consists of sequences from the gene MLIP. In some embodiments, the sequences from gene MLIP comprise or consist of:

(SEQ ID NO: 938)
TAAAAGGAAGTCTTTGCTTAATGCTTTCAGTAACTTTTAAAAGCA
GTGTACGGTGCTGGTGGTATTTGCTGCAAGGATGTGAGGGAGAGC
TGCCCAGTGAAGATAAATTTGTATGTATATTGAAATGAAATGCTA
TCTTCACTTAGAGAAAAGGGTGAGCTGATAAAGACAAAACTGCTC
ATTGGGCAAAGAAAATGCTCAGGATTGCCCTTGGCACAGTGATAA
TGTAAGATACCCTGTAATGCCTTAACAGGAGGCTAGAGGCCATCT
GCGAGTGTGGGTGTCAACATCTGAAGGAAGAGAACTTTTAGGGAA
ATATAAGAAAGGAAGGCATTACAGCCCAAAACTGAGCAAAGGAGC
TGTTAGCCTGCGTGGCATGCGGGAAAACAGTTTCTCAAATTATTA
GGCTATCCGGTGAGAAGATGACGTTCTTAGAGATTTTGAGCATTT
CAAAGTGTATTTAGAAAAAGATTTTAATCCTTTTATCTCATATTT
CTAT

In some embodiments, the intronic domain that promotes trans-splicing primarily in the heart comprises or consists of sequences from the gene ENPP1. In some embodiments, the sequences from gene ENPP1 comprise or consist of:

(SEQ ID NO: 939)
TAGGAGCAGCGTGCCTATGGAAAATAGAAAATAGGTTCTTTTTGT
TTTTGTTCTTGTTTTTTGCTTCAGAAATAATTATAGAAATCTGTG
TTTAGAAGCAGTCCTAAGAAGTTACATGCAAAGAAGTACCAGTCT
TGGAATGAGAGTGAATCATACACATACACTCCCAGTTAATATCCT
AGGTGGTTTGGGGCAAATAACTGGGCTTTCACATTCGGTCTAAAA
ATGTGATAAGACATTCTTCTTTGCTCACATAATGATATTGTAATA
TATAAATAAGAAGAAACATTAACCAAAATTTGGGTGACGGAAAAG
ATTTTGAAAAAAGTGAAGTGATAGGTACAGCTGAAATTCTGTCTT
ACCTATCAGATCTTCAACTAATATGAGTGCTACACCCATGTTTAA
CGAATTTAACCTTGGAAGTGAAAGAAGTTCTGCTCTGCATATTAA
ATTTTTTGTTAAAGTTACAGCATGTTTTGGGATTTTTTTTTTCTC
CTAG

In some embodiments, the intronic domain that promotes trans-splicing primarily in the heart comprises or consists of sequences from the gene ENPP1. In some embodiments, the sequences from gene ENPP1 comprise or consist of:

(SEQ ID NO: 940)
TAAGTATATCTCTGTGATAACTTTGAATATGGTCATATTAAGAAT
ACCTTCCTTTAGGCCGGGCACAGTGGCTCATGCCTGTAATCGCAG
CACTTTGGGAGGCCAAAGTGGGTGGATCACCTGAGGTCAGGAGTT
CGAGACCAGCCTGGCCAACATGGTGAAACCCTAAAAATACATATA
CGATACTATACTATATCTATACTAAAAATACAAAAATTAGCCTGG
CTTGGTGGCAGCTGCCTGTAATCCCAGCTGCTCTGGAGGCTGAGG
TACAAGAATCGCTTGAACCCAGGAGGCGGAGGTTACAGTGAGCTA
AGATCATGCCACTGTACTCCAGCCTGGGCAACTGAGTGAGACTTG
GTCTAAAAAAAAAAAAAAAAAAAAAAGAATACCTTCCTTTAAATC
ATTATATCGCTAAACTAAAGGGAATCACAGTTAATTTTTTTATGA
TTAAATCTCCAATGCAGAGCTTTTGTACACAAATGGATTTTATGG
GACT

In some embodiments, the intronic domain that promotes trans-splicing primarily in the heart comprises or consists of sequences from the gene VNN1. In some embodiments, the sequences from gene VNN1 comprise or consist of:

(SEQ ID NO: 941)
GTAAAGAAACAACTTGTGAAAAATTCACTAGTAAACATCAACTTG
ATTTACCTGGGAAAACTTTGTTGATGATCATTGCATAGATCCATG
ATCAATTCTTAAGTTTCAGTATAGCTTATTTTTCATCTACTATGG
GTATATTTACTGGGAGAGCAAATATGAATTATGAAGTCACAGAAG
TCAGAGCTAGAAAGTAGCTTAGAAATCATCACATTCAGTGTGAAC
ATCTCTGGTCTCTGACTCCTCACCAGTGAACAGAAAAATATTTCC
CTGTGTAGGTCTGTGATTTGAAAACTATATGAGTAAATGGCAAAA
GAGAGTCACATCAGTTTAAGATTAATAGTTTTCCTTTCTCATTGC
TAAGATAGCTGATGAGGTTAATGTAGTAAAGTCCTTAAAGTGTAA
GCTGATTGTAATCTAAGAGGTGATATGGCAGGATTTTAAGTGGTT
TAAGTCAGGTCTCGGCTACAGAGATATTAAGTGTGGTGAAAGCAG
CACT

In some embodiments, the intronic domain that promotes trans-splicing primarily in the heart comprises or consists of sequences from the gene VNN1. In some embodiments, the sequences from gene VNN1 comprise or consist of:

(SEQ ID NO: 942)
CATCTCTACTAAAAATACAAAAATTAGCCAGGCGTGGTGGCAGAT
GCCTGTGATCCTAGCTACTTATGAGGCTGAGGCGGGAGAATCGCT
TGAACCCGGGAGGTGGAGATTGTGGTAAGCCGAGATCTAGCCATT
GCACTCCAGCCTGGGCAGCAGAGCGAAACTCCATCTGAAAAAAAA
AAAAAAGAGAGAAAAAAAAATGCTTGACTAGAAGCCCAAACCTCA
CCATTATGTAACATATCCATGCAACAAACCTGCATTTGTACCCTT
TGAATCTAAAATTAGAAATAAAGAAAAGAAAAGAAAAAGAAAAAG
AAGTGACAGTGCACTGAAAAAAAAGGAAATTAAAATGCTTTGGAA
AAGAAAATAAATTATAAAAATATAGAAAACAAAATAAGATTTAAG
GGGTGTGGGGGAAGCCCAAATAGTTGTTACTCAGCCACTCAGCTC
CTCAGCTCCTCTTGCAGGCCCCCCTTTGGATTAAGTTGCATTTTT
AACA

In some embodiments, the intronic domain that promotes trans-splicing primarily in the heart comprises or consists of sequences from the gene SGK1. In some embodiments, the sequences from gene SGK1 comprise or consist of:

(SEQ ID NO: 943)
GTTCGTACTTACCGTACTATATTTTGCAGATGCCTCAAAGGATTT
GGGGTTACTTGGCATGGGGAAGGCACATAAGGTGGGGTGTAGGAG
AGGGTCTCTGGTTGTAGGTTTCTTAATTTAATGTTTGAAAACAAA
CATGCAAAAGTCTGTGTGCAGGTTGATGTTTCTGGGCAGCCTGAG
CAAAATTTGCTCTCTCAAGAGGGAAAGGAACCAGGTGGGAGCAGA
GCTAGGCTGGGCTAGGCTAGTTGAATGGTGGGACATGACATACGG
GTGGCACTGGCAATAACAAAGTCACATTCTATGAAGATTCCCTGC
AAGAGGAAGCAGACATGGGCCAGTTACTGTGATTTGAAATTGCCT
AAACATTGCTTTAGGTTGGCATGTCAATTTCAGGTACTAGTGTTT
TTTTTGTTTTTGTTTTTGTTTTGTTTTTGTTTGTTTGTTTGTTTT
GAGACGGAGTCTCGCTCTGTTGCCAGGCTGGAGTGCAGTGGCGTG
ATCT

In some embodiments, the intronic domain that promotes trans-splicing primarily in the heart comprises or consists of sequences from the gene SGK1. In some embodiments, the sequences from gene SGK1 comprise or consist of:

(SEQ ID NO: 944)
AGTAGGTTGCTGGGACAGAACAGGGGGCGCCTGCTCCACCCATCA
CGTATCCGGTTGGAAGAAAAGCCAGTCTCAACAACTTGATTCTCT
CGGTTAAATGATAAGTTTAGCCGACCATCCGTCTCCAAGTGCTGA
ACATTAGGGTAGGGGAGGAAGGAGCACATCAGACGTTCCCTTTCT
TTTCCAACCACAAATCATACGGTTGCATCTATTCTCTATTCCGCG
TAGAACCCTCCGACCTGAATGCCACGTTCCTCCCGGAATGCATGC
CACAGAGCCTCTGAGAAAATAAATCCTGTGCATGTGTTTTGAATT
TTTTCCGGACTGATTTGTGAGGATAATCAGTTAAGTTACATTTCT
TGTGTGCGTGCTGGGGGTGTAATAAAGCTAGGACACTGATGACTG
TAATTTTCTGAGTGATGTCGCAATGGGGAGAATAAATGGCCCAGT
GTTGAACTTTGCAGCTTGCTCCCTTCCTGTACTTAAATTGCCTTG
ATAT

In some embodiments, the intronic domain that promotes trans-splicing primarily in the heart comprises or consists of sequences from the gene PLG. In some embodiments, the sequences from gene PLG comprise or consist of:

(SEQ ID NO: 945)
ATATCCCCAGAGTGGGTGTTGACTGCTGCCCACTGCTTGGAGAAG
TATGTTTAGGGGACAATTGACATGAAGTCTTGTCTTAAATACTTT
TTCTGTCCTTCTTTTCCTCCTTTCCTCCTTTCCTTTCTCACTCTT
CCTCCCTTCCTTCTCTGGCTGTGACACTAGGGACCAGGCCAGGGC
AATTGGATAAGAGAGAAGGGAAGGGTTTCTAGAAAGAAACTGCAG
AGGAAAGACACAGTACAGATGATTTTGTGGGCCTGAATAAACTGC
AGAACAGAGCTGTTCACTACCATAGGCTGTATCAGTCTCTGCCCA
AACAGCCCAAGAACATTCCTTAACTGCCTGTTTCAAGCAAATCAT
GAATTTTGCTTCTTGCCACTCAGAAGTCACTAATTCTGAGTGGCC
AAGGGTGTCAGGGAGACAGCACCAATTTCATGGCACAGAGGTTAC
CTGAAGGGGCTGGACCATATTTTCCTCTTGACGTCCTCATCTTTT
CTAG

In some embodiments, the intronic domain that promotes trans-splicing primarily in the heart comprises or consists of sequences from the gene PLG. In some embodiments, the sequences from gene PLG comprise or consist of:

(SEQ ID NO: 946)
TACTCGTTCACCTGTGGTCTTCACCCCACGCTGGTGAAGATATTT
GCTTTATGTCTGGGTTTTATGGGCCATGGCCACTGCATGGCAGTG
GGGAGGAACTGTCTATCACATGAAAGGCTCAAGGGCTTTGGGGAC
AGCATCAATCTTCAACCCTAGCCCTGCCACATGCTAGCTGTGCTC
TTGAGAAAGGCAGCAGGACTCCGTTTTCTCATGTGGAAAAAGAGT
TGAAATGAGGTACTCTGTTACTCCTAGAACTCACTTAATGTTCAC
CAGTTCATACACATTCATGATCAGAGAACGATTCAGTTATTCCAG
GCTGACAATTCCCCCTTCATCATAATATGTTTAAGAGAATCATAT
AAGACTATATTTGTTTCAAAGCACTTTAAAAACCACAAGATCGAG
TTGGGTGTCTGGTGTGGGTGCCTGTAATCCCAGCTACTTGGGAGG
CTGAGGCAGGAGGGTCACTTGAGTCCCGGAGTTTGAGGCTGCAGT
GAGT

In some embodiments, the intronic domain that promotes trans-splicing primarily in the heart comprises or consists of sequences from the gene ABCB4. In some embodiments, the sequences from gene ABCB4 comprise or consist of:

(SEQ ID NO: 947)
GGTCTCGAATCCTGACCTTGTGATCCGCCTGCCTCGGCCTCCCAA
AGTGCTGGGATTACAGGCGTGAGCCACCTTGCCCGGCTGATAGTG
GTTATTTTGAAGTAATGGAATATTTTATTCTAAATTTTCTAAAAT
GATTATATATTTTATAAAGTAAAGCATTCAAAAGAAAAAAGCTGC
ACAAATTACATTTTTAGCACATTAGGAATGAAAAACTGGAAAATA
CTCAAAAGGGAACTAGTTAAACAGAAGATTAGAAAATTTAAATAC
TTATGAATTTTTTTACAGCATGGGAACCCATTTGTGTTATATTTA
AAAAGCAGAACATAACTATGTAGATTAAATGTTGATTATACAAAA
TACACATGCATAAAGTTCAAAGATTCTAAGATACTAGAAATTAAA
AGTTGATTAGAAAGGTAACATTTTCAAATTTACTTGAATGTTAAA
ACTAATTTATACAATTTTTGGGATAAGGTGTCTGTCTGATTTATC
AATA

In some embodiments, the intronic domain that promotes trans-splicing primarily in the heart comprises or consists of sequences from the gene GSAP. In some embodiments, the sequences from gene GSAP comprise or consist of:

(SEQ ID NO: 948)
GTACGCAAGTATTTCTCACTTTTATCATGGTGTACAAAGAGTGGA
AACAACTCTGTTGTAGCAAAACCCTTTTGAAATGCACTAGTTCAA
ATCAGTTCTCACCAGCTTCAACACCATCTAGCTTAATTCTATACT
CAGCATTTAAAGTTATGCCCCCCTGCATATGTACCCTTTAATGTA
TAATAAAAGCTAAAATTAAAACAAAGTTATGCCCGTTACTTTACC
CATCCTCTTTCCCTGGCACATGGTTGGTGACAGGGGAGGGACAAA
TGAGCCCAGTACTGCCAAGAAGGGGAATTTCACTTAATACAATGT
GTAACATATGTTCCTACTCTGATTAAATTCTAGGCATTAGGCCGA
TGGTCACATTCATATTTATGGGGCTTTCTACAAGGCATAGCATTT
CATGATTAGCATGAACCCTTGTGTAGTACTCAAAGCCACTGCTTA
GGACAAATGGAGCATCACAGATGAGCTCCTTGGCTGACATGCTGC
TTTG

In some embodiments, the intronic domain that promotes trans-splicing primarily in the heart comprises or consists of sequences from the gene GSAP. In some embodiments, the sequences from gene GSAP comprise or consist of:

(SEQ ID NO: 949)
TGAGGACACTGCAATTTGGAGAGGGTGGGTAACTTGTGTAGGGTA
ACACAACTAGTAATGTATAGAATCTGGACTTGATACCAGTCTTAC
TTCACAGCTCATGATCATGACCTCCATTCTGTGTCTCTTTTCTTC
TGACTGTCCACTGTTGGCTCTTGCGTTGTGACAGGCCTGGAGCAA
ACCCTGTAGCACTTTACCTTCACTGATGGATGGGGGTCTGTAAGT
GTGAGTGGTGGGGCCAGTTAGCTGGGCTGGGGAGGCTGAGCTGGG
GCACTGCCCACACAAACAGAGGATGCTTTGTTAGTTTCTGGTGCA
CATTTTCAGTCTTTCTATTTTGGGTTCAGTGCTTCAGAATTGACC
TCAGAAAGGCTAGTTGTGTGTCTGGATGCAGCCTGAATGCCAGCT
GCCCTGAAATGCTTATCTGAGTTGTGGAGGCCTAGCTGTGGGGCC
CTTGAGGGAAGGAATGGAGCAATGGTTAATGGACTGTCTTCACCC
TCCA

In some embodiments, the intronic domain that promotes trans-splicing primarily in the heart comprises or consists of sequences from the gene ABCB4. In some embodiments, the sequences from gene ABCB4 comprise or consist of:

(SEQ ID NO: 950)
ATCTGACTTGTAAAATAAGTTTCTGGTTTGTCATCATAAAACATA
GGTATGTCTGTCTCATTCTAGATCAATTTAGAAGACCCAATTTGG
GGTTTATTACGGCATTTGAAATTTGAGAGTTAGAAGTGACACCTG
TCTTGATGAAGTATTGAAACAACAGGCTAGCTCTCCCTAAGTTAT
TAGAAACATCCAGAAAAAGAACAAAATTCAAGGTAAGTACAGGTG
TAGAAATATAGAAGCCACAGACATAGGGAAATGGTCTTATGGTCC
TGAATGGGTTGGGTAGACAGGAAAGTTTGGGAATAATTTTCCCAA
GTACAAAAGAAAAATAAATTGTTCAATAGCCTCATCTCTCTTGGT
AATTTTCTAAATCATTCTATTGTGGAATCAAAGTTTTTTAAATGA
ATGAATTTCCAGCTTTATCATCCAACATCAAGAGAAAATGAAATT
GCCCTACCGAGCTAACTCTGAAAGCACATATCGGTTCCATAAAGC
AAGA

In some embodiments, the intronic domain that promotes trans-splicing primarily in the heart comprises or consists of sequences from the gene STEAP1. In some embodiments, the sequences from gene STEAP1 comprise or consist of:

(SEQ ID NO: 951)
AAACTCTGTGTTGAAAAAGTCATCTATTTTAAAATTTTTATTATA
ATTACACTAGGGCTTGGTTGTCAGTCGGTTTTATATTGAGAAATG
TTTTTTAGGCTGAAAATAAGGTACAACTTTAGCAACATAAGAAAC
ATTTTGTTCATGGACGAGGCCATATTTCAAACTTCTACCACCCTC
ACAAGATTACTTTTTAATATTGTTGTTCTTCTCCTTGTTTACCAA
TATGCATGCACATTACAGTTCTATCCAAGTAAACCATCAAATAAT
TATTAGAAAATTAATAGATTATGGAAGTGTTGCATTCAAGAAAAA
AAGTGATAGCTCCTATGTCATTTGTCTTAGAGGAATGCAAAAAAA
AAAAAAGAAAAAGTCATAAATTTGTGGAGACCTGTTATCAGGGCT
TCATAGTAGGCACAGGGAAGAGTGTAGAAGGAGATTCACATTCAG
GAAATAACTGTTGTTTGCATTTCTTCTTTCTTTATTTACCTTCTG
GTAG

In some embodiments, the intronic domain that promotes trans-splicing primarily in the heart comprises or consists of sequences from the gene STEAP1. In some embodiments, the sequences from gene STEAP1 comprise or consist of:

(SEQ ID NO: 952)
TAAATAATATATAAAATAACCCTAAGAGGTAAATCTTCTTTTTGTGTTT
ATGATATAGAATATGTTGACTTTACCCCATAAAAAATAACAAATGTTTT
TCAACAGCAAAGATCTTATACTTGTTCCAATTAATAATGTGCTCTCCTG
TTGTTTTCCCTATTGCTTCTAATTAGGACAAGTGTTTCCTAGACATAAA
TAAAAGGCATTAAAATATTCTTTGTTTTTTTTTTTTGTTTGTTTGTTTT
TTGTTTGTTTGTTTGTTTTTTTGAGATGAAGTCTCGCTCTGTTGCCCAT
GCTGGAGTACAGTGGCACGATCTCGGCTCACTGCAACCTGCGCCTCCTG
GGTTCAGGCGATTCTCTTGCCTCAGCCTCCTGAGTAGCTGGGATTACAG
GCACCCATCACCATGTCCAGCTAATTTTTGTATTTTTAGTAGAGACAGG
GTTTTCCCATGTTGGCCAGGCTGGTCTCGATCTCCTGACCTCAAATGAT
CCGCCCACC

In some embodiments, the intronic domain that promotes trans-splicing primarily in the heart comprises or consists of sequences from the gene PON1. In some embodiments, the sequences from gene PON1 comprise or consist of:

(SEQ ID NO: 953)
GTAACAATATTTACTATTTATTAAAACCAAAACCAAACTTAAAACAAAA
ACAGAAAAACATGCCAATAAATGAATTATTTTTTCTCATTCAAACAAAG
CATAGTCATGAATAATGTTCTTACTCTGGGTTTTAAACTTACCCTCTAT
GTTAATTGAACTTTATAGTAAAACCACTTAAATACTTGAGAATGCTAAA
ATATTTAAGATTTGTAAAGCATCTAATTTTGTAAAATACTAACATTCAC
ATTATCCCTATAATTACCATAATAATAATATCCAGCCCTGATCATTATA
GCTTTTCCATTTTAAACGATTTTCAAGACTCGGGCATTTACAGGAACAG
ATTTTTCTAACCCAAGGCATCTGTAAGCTATAGGTCCAGTGACAGAAAG
TCACCATGGTCAGCTTTCTGCCGGTCCCATTATCTTCAATGCCTCCTGT
CCGCTGGTTCCTCTGTTCTGTGTTCAAAATGCTCCTCTGCATGTTGGTC
CCTTTCAAC

In some embodiments, the intronic domain that promotes trans-splicing primarily in the heart comprises or consists of sequences from the gene PON1. In some embodiments, the sequences from gene PON1 comprise or consist of:

(SEQ ID NO: 954)
TCTTATATGGGAACACAATATATAAAACAACAGCTTTATTGTTTTGATT
GAAGGCTAAGAAAGGAGTAGTTCCTCTGGCCCACCAAAACTCTGTCTTT
GAGATGCTTCCAAGGACCTACCAGGGAACAGTTTAAAAGCCATAAGCAA
TATTCTCTCTGTATTCCTTGCTTATATCATTGAAGACTATTATTAATTG
CCTTTTTGGTCTGCTCTTCCATTTTTGATCTTTCTCATGGGCTGGAAGC
TGTAGTGTCGTGCACATATGACTTTGCTGCAGGGGATGAAAGGGTTTGT
GTGGGGTGGCCTAGTGGAGCATGTGGCTTCCAGAAGACTTCTCCTGGGG
ACCCATTACAGAAGCTAACTAAGAGGCTTCCTATGTGGCCATCTGGCTA
GCCCTGTGGTTTGGAGCAGTCAACATGAACCCATGGGCCATGCAGCGCC
TCTATTAGCATTTGAAGTACTGGTATTGTGCTTGATTTTTCTCCTCCAT
GGTTTATAA

In some embodiments, the intronic domain that promotes trans-splicing primarily in the heart comprises or consists of sequences from the gene TFR2. In some embodiments, the sequences from gene TFR2 comprise or consist of:

(SEQ ID NO: 955)
GTGAGTGCGCGCGCGGGCGGGGGGCGCGCGGGGAGGGGGACGCGGCTCG
GGGCGCTCACCAGCCCCGCTCCCCAGGGCTCACCCCAACACCCTGCACT
GGGTCGATGAGGCCGGGAAGGTCGGAGAGCAGCTGCCGCTGGAGGACCC
TGACGTCTACTGCCCCTACAGCGCCATCGGCAACGTCACGGTGAGCACC
CCCTGCCCGCTCCTAGGATGGGGCGCGGCACGAGCCCTTTCCTGGGTCT
CGAAGGAGGGACGCGGGGAGCGCGCCTCACCGTCCTCGTGTCCCCAGGG
AGAGCTGGTGTACGCCCACTACGGGCGGCCCGAAGACCTGCAGGACCTG
CGGGCCAGGGGCGTGGATCCAGTGGGCCGCCTGCTGCTGGTGCGCGTGG
GGGTGATCAGCTTCGCCCAGAAGGTAAGTGTCCCTGGACAGAGGGAGGG
CTTGAGCCCGGGAACCGGAGGGTCGGACTGCCAGTGAGAATCGTTCAGG
GTGCTGTGA

In some embodiments, the intronic domain that promotes trans-splicing primarily in the heart comprises or consists of sequences from the gene TFR2. In some embodiments, the sequences from gene TFR2 comprise or consist of:

(SEQ ID NO: 956)
AGTCTCCGGCGCCGCCCTGGTGGTGATGGGAGAAAATGGGTCCGCGGAG
GGCCCTGCACTGCGGCTCGCGCCTGTAATGCCAGCACTTTGGGAGGCCG
AGGCGGGCAGATCACCTGAGATCAGGAGTTTGAGACCAACCTGGTCAAC
ATGGTGAAACCCGTCTCTACTAAAAATACAAAAATTAGCCGGGCGTGAT
GGCACGTGCCTGTAATCCCTGATACGAGGGAGGCCGAGGCAGGAAAATG
GCTTGAACCAGGAAGGCGGAAGTTGCAGAGAGCCGAGATTGGTCCACTG
CACTCCAGCCTGGGCGACAGAGCGACACTCCGTCTCAAAAAAAAAAAAA
AAAAAAAAAAAAAAAGCGTCCGCGGGGAGCGCTCTTTTCCTAAACTCAG
GAACCCCTCGCCGCCCCTGCCCCTGGCGACCCCACGTCTCTGGCATCCT
TCCCTCTTCCCTCCCTCTCCTCCGGGCGCCCAGAAAAGTCCCCACCTCT
CCCCGCTTA

In some embodiments, the intronic domain that promotes trans-splicing primarily in the heart comprises or consists of sequences from the gene SLC39A14. In some embodiments, the sequences from gene SLC39A14 comprise or consist of:

(SEQ ID NO: 957)
ATCTCGGCTCACTGCAACCTCCGCCTCCTGGGTTCAAGCGATTCTCCTG
CCTCAGCCTCCTGAGTAGCTGGGATTTCAGGCACGCGCCACCACGCCCA
GCTAATTTTTTGTATTTAGTAGAGATGGGGTTTCACCATGTTAGTCAGG
CCGGTCTCGAACTCCTGACCTCAGGTGATCCACCTTCCTCAGCCTCCCA
AAGTGCTGGGATTATAGGCTTGCGCCACCGCACCTGGCCTACTTGTATG
GTTTTTAGGCTTCCATTGCTGTTTGCATCCGTCTCTCCATGGAAAAGGT
GTTTCTCTTGGATAGCAATTTTGATAGTTTCCCTAGGCTTCTCCGCAGC
ATCTGGTGAGAGATGGACTCAGCAAATGTTTACTGATGGGTTGGTGGAT
CTCAGTCTGCCCCATCACACCTGAATTCAGAACAGGGCCGCCCCAGGCA
GCAGGAGTGTCCCCACCCTCAGTAATAGAGGCCCTCGTCTGTTCTTGTT
CCTCCACAG

In some embodiments, the intronic domain that promotes trans-splicing primarily in the heart comprises or consists of sequences from the gene SLC39A14. In some embodiments, the sequences from gene SLC39A14 comprise or consist of:

(SEQ ID NO: 958)
TGCAGTAGAACAGAAACTTGCGCGTCCTCTTTCCCTGGGGCGGCACAGC
TGAGCCAATCCCCTCCTCCCCTACCCTGGGCTCCAAGGCACCCAATGAC
TGCCACCTGAGAAATGAACCTGTCCCTCGTATCAAAGCCACAAACGACT
TCTAACTCTGGATCTCAGGGCCCACCTCTCACTCCTCTGGCCTCTGCAG
GTGCTGTCAGATGCAAGGTTCTTCCAATTTCCCTCCTCCTGGGGACTTG
ACAGTCTCCCCTCCCTTGTTAAGCCCCATACATAAGGCATCAAGGGAAA
GCAGCAGCTATCGACTGCCGTCCATGCCTTTGGTGCTAAGTACTTGCTC
TATTCTAAGGAGAAATCCTGGCAGTACTCAGGTCTGGGCACCAAGAGCT
TATCCATCTGGAATGAGATGGAAGGAAAAGAGGTTACCAAGTGATATGT
AGAATTGGCCTAAAAAGCGAGTGTTCTTTAATTTTGAGCAGGCCATCGT
TTCTAGCAT

In some embodiments, the intronic domain that promotes trans-splicing primarily in the heart comprises or consists of sequences from the gene RBPMS. In some embodiments, the sequences from gene RBPMS comprise or consist of:

(SEQ ID NO: 959)
CCTTAGGAGTCGCCCAGCAGCGAGACAGCTGTGGATCCCCTCCAGGCGC
ATGGGAGGCTCACAAGGCAAGGACTCTGATGGTTTTGGCTTCCACCTCC
TGTACGGTGGCCAGTGCTGTGAGGGTGGTTGGTCCCACAGGGAAACCTG
TGAACAGAGTCATGTCTGGGGCCCCAGGGACATGGCTTCAACCCCTCAC
CTGGCAGTCCTGCCTCCCAAAAGTAAGGCAGGAGTCCGGAGATTCCCAT
TGCTTGCTTCTGTGAATCTCAGGTCACTTTAGCACTTGACCCCTGACCC
CAGACTGCCTTGTCCTTGAGGCCTGGGATTCCTAAGGTATATACTGTCT
GCTCCAGAGCTGTGGCTGTGGCTATCCGGAAAACGACAGCTTTGAAGGA
CTGCAGAGCTTGCCTTTGGGGCTGTTCTGCCCAAGGCCTTCCATTGTTT
TCAGACATAAATGAAATTGTTAATCCTCTGACATTTACCTTTTCCTCTC
AACCTGCAG

In some embodiments, the intronic domain that promotes trans-splicing primarily in the heart comprises or consists of sequences from the gene RBPMS. In some embodiments, the sequences from gene RBPMS comprise or consist of:

(SEQ ID NO: 960)
TGAGGCTTTCTAGGAGCACCTGGCTTAGCTCTCACAGGAGGAGGGGCGG
ACGGGGAGGCCAGGCCTCATGCTCACAGCGCCAGCCTCCTGTGAGAGTG
GGCTGGGTCTCCTTTCCTTTCCCAGGCCCTTCCTGGAGGCGATTTCTTT
CTTTCTTTCTTTCTTTCCTTTTCTTTTCTTTTCTTTTCTTCTCTCTCTC
TCTCTCTCTCTCTCCCCTCTCTCCTCTCTCTCTCTCTCTCTCTCTTTTC
TTTCTTTTCTTTCTTTTCTTTCTTCTTTTTTTTTTCTGAGGCAGAGTCT
CGCTCTGTCGCCCAGGCTGGAGTGCAGTGGTGTGATCTCGGCTCACTGC
AAGCTCCGCCTCCCGGGTTCATGCCATTCTCCTGCCTCAGCCTCCCAAG
TAGCTGGGACTACAGGCGCCTGTCACCACGCCTGGCTCATTTTTTGTAT
TTTTAGTAGAGACGGGGTTTCACTGTGTTAGCCAGGATGGTCTCGATCT
CATGACCTT

In some embodiments, the intronic domain that promotes trans-splicing primarily in the heart comprises or consists of sequences from the gene DPYS. In some embodiments, the sequences from gene DPYS comprise or consist of:

(SEQ ID NO: 961)
GTAAGTCTAAGTTCTTGTATTCCTTGGCTTCATTGTGGAGAAAATGCAC
TAACTTGCACAGAAATAATGCTTTGGCATTAATCAGCCGCTTCTCCTTA
GATGACTTCTTTAGCTGTGCTCAGGTATTTTGATCCTGTACACAAGATA
AAGGTTTTGATGTGCAAATATTCCTTTGTTTTTCTTGGCATAGAGGATG
TAGAAGTTGTGTTGCATTCTCTGACAATGGATCTGTATATAAAACCAAT
ATGAATTTACCACTTCAGTGCCTGGCAAATGCCCGAGTTCACATGGAAG
AAAGGTAAAGCGTGATGAAGGAAATAAAAGGCTTAGTTTGAAAAGGGTT
GAATTGAAAACTCAAAATAATTACAGCCTTTTTAAAGCTTTCAGATAGA
AAAATTCCAGCACAAAGGATGTCACTTTCAGTTAGCCAAATTTTCCAGT
TGAGTTCATCTTCAGCCTATCATTCCTAGTAGATTCCAGCTTTTTGTTT
CCAATTTCC

In some embodiments, the intronic domain that promotes trans-splicing primarily in the heart comprises or consists of sequences from the gene DPYS. In some embodiments, the sequences from gene DPYS comprise or consist of:

(SEQ ID NO: 962)
GCACCACTGCACTCCAGCCTGGGCAACGAGAGTGAAATTCCATCTCAAA
AAAAAAAAAAAAAAGTGTTGTCACCTATTCTCACTCTATAATATTCTAT
CTAATGTCTACATGATTATCTCATAAATCTTAGGTCTGTAATAAATAAG
TCAGATCATAAATGTGGTATTGTCAAACTTTAAAGGAGAATCGAAAATA
CCATTACCAAAATTTAGCTGTCACCTATAACAATTTTTGTGATTCTAGG
ATGAATGGGAAGAATTAGTGTGGATGCTAAGTAGTGGGGTTTCTGAAAA
GTCATCTTCAGGCTTCCATATGTTACCCCATCATCTCCATCATTGGCAT
TATGACTGTTTAATACTTACCAGCATCCTCAGATGCTCTACAATATAAG
AAATTAGGTGCAGTTCTGTTTGTTTAAGAGATGCAAGTCTTGTCATTTT
ATCGATAGGATACTTTTTAGTATTTGATCATTAGAATGATTCTTTGAAT
TGCTTTACA

In some embodiments, the intronic domain that promotes trans-splicing primarily in the heart comprises or consists of sequences from the gene RCL1. In some embodiments, the sequences from gene RCL1 comprise or consist of:

(SEQ ID NO: 963)
TATTGGGATGGGTGAGAGGAGGAAGATGCTTTTTGGGAGTCAGATCTGT
TTAATTTTGAGCAAGTGTCTTAGTTTTTTTGTCTTAGTTGTCTTTAATG
TAAGTCCTAGAACTCGGAGCCATGGCCCTCAACACAGGGTTACTGTGAG
CATAAAGTAAGGTGATAATAGATGTGAAAGAACTTTGGGAGCCGTGAGC
TCTGTGGGATTATGTGCTAGGATGGTGACATCTCTTGTGCTTTCCTGGG
GACAGTGGGCAGCCTCAGACCTCAGAAGGACAGCACCTGCTTGGTCCAA
AGGGTATGAGATTCAGGACTGTTTGTCTAAGCTGTCTGTGGTCAAAAGT
ACATTCTGGACTGAAGAGATGGTGATGTAACTGGGCTGTGTGTGTATTG
AAAAATCCCTCTTTGCTCTTTCCTCATTTCAGGTTTGTCCTTTGAGTCA
AGTTAAATATTTATTGAGCATTTCTGAGTACAAAAGTGGTGTATTGGAG
AGTTGGGAG

In some embodiments, the intronic domain that promotes trans-splicing primarily in the heart comprises or consists of sequences from the gene RCL1. In some embodiments, the sequences from gene RCL1 comprise or consist of:

(SEQ ID NO: 964)
TAAGTTCAAGTGGAGCTAACACTTGGGTGCCGGCTGGAGGGTTTCAGGA
GAGAGATTGGGACCTCGGGCTGGGAGAATGGGGCAAAGCAGCTCAGGTT
GAGGAATTTGGTCAGTTTGGATGGGTGGAAGGAGTTGATGGTGCAGAGC
TGAAGATGGACACAACTGAGCAGGCTGGGGGCTCTAACAACAAGATATG
GGAGGACCAGGAGCCACCGAGAGACTGTCACCAGCATGAGGAAGATTGG
TCTTTTAGTATGAAGTTCAGGATGGGTCAAAAAAGGGAGCTAGATACCA
GATCAGGTCATGTATCGTCTCTGAGCACTTTGTTTCTGCAGTTCAGATG
AAAATTAAATATGTTATTGTCAGCAGTTCTTGAACATTGGGCTGGATTG
GGCAGATTATATACCTCGTTCTACCTGTTGGACTTGTCACGTGGGGTTT
GTATCAGGGTAGTGGGAAGGTCACATGTTATTTCTCCATTTTTTTAGTT
CCAAATATA

In some embodiments, the intronic domain that promotes trans-splicing primarily in the heart comprises or consists of sequences from the gene TOMM5. In some embodiments, the sequences from gene TOMM5 comprise or consist of:

(SEQ ID NO: 965)
GTAGCGGGGAGCAAGGAGGGTGCGCGTTGAAGGCGACGGGCATATGCGC
GGTTTGAGGTCTGCGGGTCAGGGTCCGGGCACTGAAGTGCACGTGGTGG
CCGGCGCGCGTGTTTAATCATCCTGGTTCTCCCAGCTTGAAGAATGTTT
GAGCTGCCGGGCGCGTTGGCTCACACCTGTAATCCCACCCAGCACTTTG
TGAAGCCGAGGCGGGCGGATCATTTGGGGTCAGGAGTTCGTGGTGGCGG
GCGCCTGTAATCCCAGCTCCTCGGGAGGCTAAGGTGGGAGGATTGCTTG
AATCCAGGAGGCGGAGGTTGCAGTGAGCCGAGATCGCGACACTGCACTC
CAGCTTGAGTGACAGAGCAAGACTCCATCTCAAAAAAAAGAATGTTTGA
GCTGAAAGAGCTCAAGTAATTATCCCTAGAGGAACTCCCTCGTTTTTGC
AGACAGAGAAACAGAGAAACAGAACCATAGTTCTAGAAGGAAGAAACAG
CCTTCTTGG

In some embodiments, the intronic domain that promotes trans-splicing primarily in the heart comprises or consists of sequences from the gene TOMM5. In some embodiments, the sequences from gene TOMM5 comprise or consist of:

(SEQ ID NO: 966)
AGGCTGGAGCGCAGTGGCGCGATCTCGGCTAACTGCAACATCCCCCTCC
TGGGTTCAAGCGATTTTCCTGCCTCAGCCTCCCGAGTAGCTGGGATTGT
AAGCACGCGCCACCGAGCCGGGCTGATTTTTGTATGACAGATGTTATTA
AAGACAGCGTGGAGAAACCAGAGCCTGCGTTGCTGAGCTTGCGGCCGCC
CGCTTCACACCGCACTTCAGATCAGCTGGGTCAGGACGTGGATTGTCCC
GTGCAGGCATCCATCGTCGAGTGTGGCAACGAGAGGGCGGGAACAGGAA
GTGAGGATAGGCCGAGTTCCGGGCGCGAGGCGGCCACCGTGGAGAGCAG
AGCGCGGCGGCTGGAAGCTGCTAAGTCAGAGCCGCGATGTTCCGGATTG
AGGGCCTCGCGCCGAAGCTGGACCCGGAGGAGATGAAACGGAAGATGCG
CGAGGATGTGATCTCCTCCATACGGAACTTTCTCATCTACGTGGCCCTC
CTGCGAGTC

In some embodiments, the intronic domain that promotes trans-splicing primarily in the heart comprises or consists of sequences from the gene CBWD5. In some embodiments, the sequences from gene CBWD5 comprise or consist of:

(SEQ ID NO: 967)
GTAACTAACCATCCCAGTCACAAAATGCCGTGAACTCTGGTGTCA
TGGGATTTTGCGGGCTGCCGGTGCCTCTTCTCCTGAGGCATTGGG
GCCTCATGATCAAGAGCAAATGTCTTTGAGGTAGGGGCTGGGTCA
CTTGCTTCTCGTATGGGTTACCTCTCATCTTGGGAGTTAATGATG
CCCGGTCCCCTCTGCGTTAGATTCCCATTTCTCAAGTGAGATGGA
AAGTTAGCCTTTGAAACAAGGCTTTGAAGCTAAGACACGCCCCCC
TAATTGTCTCTGTGACCTCTTACCAAGTTATCTTACCTTCCTGAC
GCTCATCTACCTCACCTGAAAATGGAAAAATACCTGTCACCTGTG
AAATAGGGCCGGAACAACCTACCTATTGCCATTAAAGGATGTCCC
GTCTGTAAAATGTCCGACACGTGGGGCTTTCGTCAGCAGATTCCC
CTTCCCCAGGAGCCAAAGTCCTCCAGCCAGTTGGCAGGAAGTGCT
CCTA

In some embodiments, the intronic domain that promotes trans-splicing primarily in the heart comprises or consists of sequences from the gene CBWD5. In some embodiments, the sequences from gene CBWD5 comprise or consist of:

(SEQ ID NO: 968)
CTGAGGTCCAAATAAATAATGAGGAAAAAGTAAATAAAAGTGATG
CATGAATACTAAAAATTTGGAATTACATATATAATATGTAATAAT
AAAAAAAGAAAAAATTGTCACTGGTTATCCTTCAGGTATAAAAAT
TACAATTAAAATTGTAATGTTATAACCTTCCATTCTTTCCCCTAT
GAATTAAAACTTTTTTTTTTAACGAGACTAAAAAATCAAGTTGAT
AAAATCTATGCCCTGTGGGAACTGGTTAGGTCAGGTTACCCACTC
CAGATTTCCAGCAGGCTAGCCTCAGCTCTACCTACAGCCAGTAGC
TTATTAAGGCTCTAGGCGCCAGTGACACCTTCAGACAGGTGGTTA
GTGCGCCCTAAGGGCTCTCCGCAGCGTCGCTCAGGTTCACAGAAC
ACGCCCAGGGGCGTGTCCAGCTGTCGTCGGGGAGAGCCCACCTCC
CCGGGGGGTGTGGCTAAGGGACGAGGCAGTTCTCGTCCAGAGCCC
AGGT

In some embodiments, the intronic domain that promotes trans-splicing primarily in the heart comprises or consists of sequences from the gene TJP2. In some embodiments, the sequences from gene TJP2 comprise or consist of:

(SEQ ID NO: 969)
AGAAAAGCAAGACAGACTGAGAAAGTGTGGCTTAGCCGCGGTATG
GAGGATGGATGGTAGCACAGATAGGCTGCAGGGCCCCTAATTTGA
GTTCCTGCACAAAAACGACCATAATATCAACATTGTACCAAAGCC
AAGTGATCTCAGGGAGAAAAATAAAAGATGCCTTCCCTGGCGAGC
ACATCAAGGTTTCAGTCGCTTGGCTGTGTCCTTGGGAATTTTCTT
GAGTCCCCACTCCCAGGAGCCACATCAGACCCCACTGACCGTCCA
ACACAAATTCCTCTAGATGGAAGGGATGGATGATGACCCCGAAGA
CCGCATGTCCTACTTAACCGCCATGGGCGCGGACTATCTGAGTTG
CGACAGCCGCCTCATCAGTGACTTTGAAGACACGGACGGTGAAGG
AGGCGCCTACACTGACAATGAGCTGGATGAGCCAGCCGAGGAGCC
GCTGGTGTCGTCCATCACCCGCTCCTCGGAGCCGGTGCAGCACGA
GGAG

In some embodiments, the intronic domain that promotes trans-splicing primarily in the heart comprises or consists of sequences from the gene TJP2. In some embodiments, the sequences from gene TJP2 comprise or consist of:

(SEQ ID NO: 970)
GCATAAGGAAACCCAGCCCAGAGCCACGAGCTCAGATGAGGAGGG
CTGCTAGCAGCGATCAACTTAGGGACAATAGCCCGCCCCCAGCAT
TCAAGCCAGAGCCGCCCAAGGTACGTGGCTGGGAAGCCCAGGATG
GGAAGGAAGAGGAAGCAGATGCCTCTGAAGCCTCCTGGACGGCCA
GGGAGGAGCATGCACACTGAGATGGTGTTTAATTACGGTTCTGAC
TCACTGTGTTCTCTATTAGAGCCTATCTTTTGGCCTGTTGGGTGA
ACTCCCAAATTAGGGAAGGGCAAAAAGGAAGGAAAGGCAATATTA
GGAGAGCATATGATTTTAGGACTGTTAGTGTTTATTTAATTTACT
CTTCTTGTTAAAAAAAGATTGTAAAGAAAAAGATTGAAAAATACT
GTACTGTATACCCAGAATCACCAATCGCAGAACGTGTTTTTCTTT
TGCTGTCCAGCTAAAGTTTTCTGCCCAGTCTGATTTAGATGCACC
AGAT

In some embodiments, the intronic domain that promotes trans-splicing primarily in the heart comprises or consists of sequences from the gene ALDOB. In some embodiments, the sequences from gene ALDOB comprise or consist of:

(SEQ ID NO: 971)
GTAAGGATATTGCCCACTGAGATGAATCATGCCAGTGTAAGCTAG
AAATGAAGGCAAGTAAAGAAGGAAGCAAGGAAAAACAAAACACAA
CTATTACACTGGTCTTAGAGCCACACGTACCTGGTTTTCAATACC
AGCTCCTCCATTTAACAACTGTGACCTTGGGCCAGTCACTGAAAT
ATTGTGAGCCTCAGTTCTCCTATCTCAAAATGGGGATTTCTCAAA
ATGTTGTGGGGATTAACTTAGACAATGTACATAAGGTGCTTACCA
CAGTACCTTGGACAAAGAAGATTAAACTCTGCACCCGGAAGTATT
ATTTTTAGAAAAAAATTAGCAGCAGTAGAGTGGAAAGTCCCCACA
CTTTGAGGTCAGACACACCTGGGTTTGAATCCTGGTTCTATCACA
TGCTACCTAGATAATTTTGCAAGGTACTTCTGCCCAAGTAGACAA
GCATTAACTCACAACATTGGGTTAGCTAAGCAGAATTTGAATCCC
AGCT

In some embodiments, the intronic domain that promotes trans-splicing primarily in the heart comprises or consists of sequences from the gene ALDOB. In some embodiments, the sequences from gene ALDOB comprise or consist of:

(SEQ ID NO: 972)
ACTTCTCAAAAGTGAAAACTAGTATCCATTGAATAAGAGTTTTTA
CCATTCCAAGCATTCATTTAATTCTCATAACCTCTGGTAGTAGAC
TCAATTATTACCCTTATTTAGAGATGAAGTGAGAAAACTGAACTA
CGAAGAGTAACATAACTTGCCCACACAACAAGGCAAGGTCAGAGC
CAGTTTAGGCTAATTCTCAAGCTCATGACTTTTGCCTTTGCTATG
TAGAGAGTAGAGGTTGTATCCTTAATTCTCTTTACCTCCTGTACT
AACACAACACCTGGTGCATGCAGGTGCTCAATAAATGTTAGGGTA
GACACTCTCACAGTGTACCTTCTAGGTAACTTCCCTTCTGCTATG
GACTGAACTGGGTCCCTCGCACTAATACACTTTACTAATTCAACT
CCTTGATCTGTAAAAGATGCAAACTGTTAGTTAGTTCAAGAGTTC
TGCTTGTGTTTTCCTGACATCTGACACCTGTTAATTATTTGTATC
CACA

In some embodiments, the intronic domain that promotes trans-splicing primarily in the heart comprises or consists of sequences from the gene PTGR1. In some embodiments, the sequences from gene PTGR1 comprise or consist of:

(SEQ ID NO: 973)
GTAAGTAGACCTCCTGCTTTCTTTCCTTCCCTTTTCTCCCTCCTC
TAACCCCCTTGCTTTCTTCCTGCCAAGTTTCTAGACGCCCCAGTG
GAGGAGGTTTGCTCTTAGACCTCCTCCCCTTACGTCGAAATGACC
GCTGGGTCTTGCACTCGGAGGATCTTGGGAGCCGACGTCCCGACG
CCTCCCGCCCCCGCAGTTCCTTGGAGAGCTTGGAGCCGCGCGCCG
GAGGGAATAGGAAAGCTTGGTTACAACCCGGGACACCCGGTAGCT
TCCTGCACCCGGGAGGGGGCTGGCCAGGGAACTTGCTGGACTGCA
CAACTGGGGGAGGGTAGAAGAAGAAAGATCGGGGTGGGGATTCGG
GAATGGGCAAGCGGGCGGCGGGCGGCGGGAGGCGAGGGTGGGAGC
AAAGGGCGAGCGGGCAAAGGCCGGGCGCCGCGCCAGGCCCCCTCC
TCGGGCGGGCGAGACTTTCCCGGGACCTGGCCCCTCTGCGGCCAG
ACCC

In some embodiments, the intronic domain that promotes trans-splicing primarily in the heart comprises or consists of sequences from the gene PTGR1. In some embodiments, the sequences from gene PTGR1 comprise or consist of:

(SEQ ID NO: 974)
GAAGCCAGGTCAAGGCCTGATGTTTGCTAGGGTGTGATGGGTGGG
GTGCCTCAGTGGCAAGAGTGCTCACTCCTGCCTGGGGGAGTCTGG
CAAGGGTTCACAGAGGTGACCTTGAATTGGCTGGTGGAAGGCGAG
TAGGTGTTCAGCAGGCGGACTGGGGACAGACAGGCAGGGTGTCTG
CACCGCAGCCCTGGGCGAAAGCTTGCACTGCGGGCAAGCGGCAGG
GCTGACGGGGAGCCCAGGGATCGGAGCCAGCACCGGGGGCTTATT
AGGGCTGCGGCCGCCCCAGGCCCTGCCTTTGGACCCTTCCGGCCT
GCTTGGCAGATGCCACGGTTGGTTCCCCCACGGGGCATATTTCCG
CCGGGCTCCAACTTAGGACCCCAGGCGGAGGTCCAGTGTCAGCTG
GACAAAGCTCTGTTAGGAATTTTCCTATCAGGAGTCCCTAACCCC
CTACACGCACACACACGGGCGCGCGGGCGCGCACGCACACACACG
CACA

In some embodiments, the intronic domain that promotes trans-splicing primarily in the heart comprises or consists of sequences from the gene ORM1. In some embodiments, the sequences from gene ORM1 comprise or consist of:

(SEQ ID NO: 975)
AGCCTCCTACCTCTGCTGGAAGCCCAGATCCCATTGTGTGCCAAC
CTAGTACCGGTGCCCATCACCAACGCCACCCTGGACCGGGTGAGT
GCCTGGGCTAGCCCTGTCCTGAGCACATGGGCAGCTGCCTCCCTT
CTCTGGGCTTCCCTTTCCCTGCTGGCTGTGGTCGGACCCCCACTC
CCGGCTCTGCCTTTTTCTCTTCTGGGTCCCCAGGGTGAAATTCTC
ACCAGCCCAGGGGACTCTGGAGGCACCCCCTGCCTCCAAACACAG
AAGCCTCACTGCAGAGTCCTTCACGGAGGACGGTTCTGTGCTGGG
CCTGGAGGGGCTGCCTGGGGGGCAATGACTGATCCTCAGGGTGAG
CTCCTGCATGCGCACTGCCCACCAGGGGCCTCATCTCCCCATCTG
CAAAATCAGGGAGAGATCTGCCTGAGTCTCCTCCCAGCTGACAGT
CAAAGATTCAGCATCAAGCCCCCATCACCAGCTCCCACCTTCTCC
CCAG

In some embodiments, the intronic domain that promotes trans-splicing primarily in the heart comprises or consists of sequences from the gene ORM1. In some embodiments, the sequences from gene ORM1 comprise or consist of:

(SEQ ID NO: 976)
TGAGAGCCCCCATTCCAATGCACCCCCATCTCAGCTTCTGGCCAG
AAGACCTGAGCAAGTCCCTCCTTCTTCCTGGCCTTGGCCTTCCCA
TGGGTGGAACCGGGAGGGCTGGCTTTAATCTCCACCAGACTCTTG
CCCCAGGACTGTGATGGGCGATTGGCCACTTCTCAATAATCTTCC
TGTTTTCCTTCCGCCTTCTGTTTGGCTTTAGACAGGACCAGTGCA
TCTATAACACCACCTACCTGAATGTCCAGCGGGAAAATGGGACCA
TCTCCAGATACGGTGAGGGCCAGCCCTCAGGCAGGAGGGTTCACT
GTGGGAACAGGGCAGGCCAGCATAAGGTGGGGGCTGGATGTAGAG
CCCTGGAGGCTTTGGGCACAGAGAAATAACCACTAACATTTTTGA
GCTCTAACGATGTGCTCAGAAACAACTCTAAGAGGACACTGAGAG
AATTAGAGGAGGAAAAAGAAGAACAGAGACCTCAAATAGTTTCCC
CAAG

In some embodiments, the intronic domain that promotes trans-splicing primarily in the heart comprises or consists of sequences from the gene ORM2. In some embodiments, the sequences from gene ORM2 comprise or consist of:

(SEQ ID NO: 977)
TAGTTTCCCCAAGGTCACACAGCTTATAATTAGAACTAGAATTGG
AACTCCAGGCTGGCTTCAGATCTGCCTCTCTCTCACGCCCTCTTT
AAGATCCTTTGCAAACCAATGGTAGAAGCCTGTATGTTGGAGAGG
TGGTACCTTCAACTATGTCCCCCATCACCGCAGAGGTGGCACATG
GCAGGGATCTGATGGAGCTGAACTGACATCATTTAGCATCCCGAG
CCTCCTCTCTGGGCCTCATTTTCCTCCTCTGTAAAACGGGGAGAA
AGGCCCTGACAGCCACAGTCTGTGTGAGGCTCCTGAGATCTCATG
TACAGAAAGTGCTTGGCGTGGAGCTGGGCACGCAGCAGGGGCTGG
GCACACGGTGGCCCAAAGGAGACCCGGGCCTTCACTGATGGGCTT
TGTGGCCCCGGACACACCTAGGACTCCTCACCTGTAAGACAGGCA
CCATTGTGCCATCCCATGTTCTCACCCAGAGGCTCTTTTTCTCTT
CCAG

In some embodiments, the intronic domain that promotes trans-splicing primarily in the heart comprises or consists of sequences from the gene ORM2. In some embodiments, the sequences from gene ORM2 comprise or consist of:

(SEQ ID NO: 978)
TAGGCATGCTTAGCAGCCCCAAACTCATGCCCCTCTCAGGCCTCA
CCCCCCATTCACCCACCCCTGGGCTGGCCCCTAGAACCCCAGCCC
TCCCTGGCCTCCCGCCGGGCCCCACCATGTCCCCAGTCAGTCTCC
TTGCTCCCCCTGCAGCTGACAAGCCAGAGACGACCAAGGAGCAAC
TGGGAGAGTTCTACGAAGCTCTCGACTGCTTGTGCATTCCCAGGT
CAGATGTCATGTACACCGACTGGAAAAAGGTAAACGCAAGGGATT
GGACAGTGCCCACCTTGTCCATGGCCCAACTTGGGCAGCCCCAGA
GGCCCAGAGCAGGAAAGCTGCCAGGCAAGGCTGCACAGCTAGGCA
GATCTTCTGCTTTTAGGCACCTGCCTCACTGTAGGGACAGCTGAG
CTCTACAGAGGCCCAGGGGTGGTGGATGAGAGCCCAGGAGGGAGA
AGTCCCTGTGAAACCAGGGAGGACCTGAAAGCTAACAGGAGGGAA
CAGC

In some embodiments, the intronic domain that promotes trans-splicing primarily in the heart comprises or consists of sequences from the gene C5. In some embodiments, the sequences from gene C5 comprise or consist of:

(SEQ ID NO: 979)
GTAAGTAAAACAAGCTATCTTCATTTTTCAATGCATCTTAGTTAT
GACGAATGAAGAGTTAAATTTAACAGATTCTGTTAACTTGAAAAT
AACTTGCTACTTCTGAGAACAGCAAGTGAATAGTATCATCTCTGA
ATGAGAAAGTCATGGAAGCATTTTGATTTGATGAGTAGCTAACAA
CATGACAGCTTTTATGGAATTGTTATTTATTAAAATTTACGTTAA
GTGATTATTCAAATTCCTCTTCAAAAAAGCACAAAAGAAACTCCA
GATAATAAACTTTCTTGACATTTTTTGCTTATTTTAACTTGGTGT
TTAGTTTTCTTTGTGTTTTTCATAGACTATACACAGTTAAACCAG
GGTTATTTAGGAGTCAATCTAATACTGTGATCTGAAAATCTGTTG
TTGTAGTGACTTCTACCATACATTTATGAGCTGACTTTTCACAAT
TTTTAGTGTCATTATTGAGATAATATGCATTTTAAGTAATGTTTT
AAAA

In some embodiments, the intronic domain that promotes trans-splicing primarily in the heart comprises or consists of sequences from the gene C5. In some embodiments, the sequences from gene C5 comprise or consist of:

(SEQ ID NO: 980)
CTAGGTTATGCTCAGACCATTTGCAGCACCAAGGACACTGCCATT
CAGGTCACCAAAAAAAGATCATCTTGTGAGCCCTGTCTAAACCAG
GCCCCTTCTAAAATCATTTCAGTGTGAAACTGAGTCCAGGTGGGT
CTTCATCAGTATTAGAGTGGACCTGCCAGATGTATGCTTGCAGAT
GATGCAACATCAAAGCAAATGCCCTGGGCAAGAGAAGAAAGGCCA
AAGCAATGGGCTTACTGGTTTCTAGCTTTGAGCCACTCTTCATAG
TGCCAAATTCCCATTTTAGGGTCCCTTTATTTGTATTAATAAAAA
TAAATTCCCTGGGTGTAAGTTCCTGTAAAGTACTCCCTATCTGTA
TTCCTGGGCATGAAAGGGTGTTTTAGTTCCAATTCTAGTTTTCCC
ACTTAGAGGTTCACCTTTCTGACATTAATAATTAACCAGATTATC
TGAATTCTTCAAGTTCAGTTTATTTAAAAGGAGACTATCCTCAAA
AGTG

In some embodiments, the intronic domain that promotes trans-splicing primarily in the heart comprises or consists of sequences from the gene DMD. In some embodiments, the sequences from gene DMD comprise or consist of:

(SEQ ID NO: 981)
CGTCTTCACCTCTTTTCAACCTGCTCCTCTTTTTTGCCTTAACTC
TGATGGCAGTCACACAACTGTGTGTTTTTCTTTTAATTCACTTTA
CACCTTGGTTTGGCTATTGCTTTCCATGGTTCATACTTAATTGGC
TGGATTTTATTTATTTTGACCTTTCAGTAAATTTTTTTGCGGCTG
AGTTTGCGTGTGTCTCCTTCACCACCTCATTTTTTGTTTTGCAGT
CCCGTTACTCTGATCAACTTCTGGCCAGTAGATTCTGCGTGAGTA
CTTTTTTTGCTGAAGGGTGCTGCTACCACCAACACATTCGCTCGC
TTGGTTCTCTTTTGTTCTGTTTATTTGTTTGTTTTCTTTTGTTTT
GTTTTGTTTTATGGACATATTAATTCCCCTTCCTTCATTTCAGTT
TTTTTTTTTCTTTTCTTGATGAGACTTTGAAAGTACCATCTACAC
AGATCAGGAGAATCAGAGTGGGTTTTTTGGTTCCTCCACCAGGTG
AAAT

In some embodiments, the intronic domain that promotes trans-splicing primarily in the heart comprises or consists of sequences from the gene DMD. In some embodiments, the sequences from gene DMD comprise or consist of:

(SEQ ID NO: 982)
AAAATACCATAAAGTGTGGTAAAATTTAATGTCACCTTTTTGACC
TTAAGACCTATAAACTACCATTTATTGAGTAGCCTAGTAAGCTTG
TATGTTTCTTTTATTTATCATAACAACGTGTTGAGGTGTTGTTGC
TCCCATTTTACTGATGATCAGAGCCTCTGAGAAGTAAATAACTCA
GCCAAATTAGTACATGCCAGGCCTGGTTTCAGAGCCCCATTTGTC
TGATTCCAAAGCCTTTGCTCACAACCACAACACTCTCCTTAATTA
CAAAACAAGTGTCATGGGGCAGAAGACTGGAGTGGTCATTAGTTT
TGAAATCATCCTGTCCTAAATCTGATCTCACCATGATCTCCCTTT
TAGACTACATCAGGAGAAGATGTTCGAGACTTTGCCAAGGTACTA
AAAAACAAATTTCGAACCAAAAGGTATTTTGCGAAGCATCCCCGA
ATGGGCTACCTGCCAGTGCAGACTGTCTTAGAGGGGGACAACATG
GAAA

In some embodiments, the intronic domain that promotes trans-splicing primarily in the heart comprises or consists of sequences from the gene GJB1. In some embodiments, the sequences from gene GJB1 comprise or consist of:

(SEQ ID NO: 983)
TTGCCCAGGCTGGTCTCAAACTCCTGGCTTCAAGTGATCCTCCCG
CCTTGGCCAAAGTGCTGGGATTACAGGCATGAGCCACCATGCCCG
GCCCTGTCATACAGTTTTAGAGGCTGGGAGCTAGAAGGGTTGACC
AGGATGGCAGCCTGTACAGGATCCCTCTCTGCAGCTTGCCCGCAC
TGTGGATCTGCCTAAGGCCAGGGCATGGGGGAAGCCTGAGGGATG
CTGAGGAGCACAGGTCCAGCTCAGTCTCCCAGAGAGACTAAACAT
GGGTGGGTGACAATGATGCCTCTCTTCCTCTGGGACAAGGCAGTG
ACTCCAGGCTATGTGATCTGTCTCTCCAAACCCATAGGGAGCTCA
GAGGATCAATCTTGTTTAATGATTGTATGTGGGAGGAGGTGGGGC
TGGTTAATGTTTGTCTGGTTCCTCTTGGGCTGTGGGCCATTTCTG
GCTCTGGCCCTGGGTTCGTTGTCCCTCCCCTGGGCTGGGCTGACA
GTAC

In some embodiments, the intronic domain that promotes trans-splicing primarily in the heart comprises or consists of sequences from the gene GJB1. In some embodiments, the sequences from gene GJB1 comprise or consist of:

(SEQ ID NO: 984)
TGAGGAGACTGGGGGCTGAGGGAGGTGGGCCAGGGAGGGCTGATG
TGGCACAGTGGGCCGGAAATAGGGGCCGGGTCTTGCCTTCTACTG
ACCTCCGCCTTCAGCCACTTAGGTCCCTTCCATGCTTCCCTCTGA
AGCTGGGGTGAGGCCCCTCCTGCCAGGTGCCAGACTTGCTTAGCC
TCTCTCTGCAGCCTCTTTCCAGGTTCAACATTGCTTTTCCTCTCA
CTTTTTTTCCTCTGGCCAAAGATTCCCCTTATTCCCATAGGGCCA
GATTCTCAGGAAGACTTGAGGGACCCCCTAGGTGCAAGGTGGCCA
AGTTTGTCTCTCTGAGACATTTCCTGCCACACAGGGTTAGGGTTT
AGTGGGAATGTTGGGCACTGCCAGGTGCTGTTTTCCTTCCTTGAT
TACAAGGCAAGCCAGGCATGCACCCAACACGGGGCTGCGAGTGGA
TGAATAAAGCGTGCTCAGCGGGCTGCAGAGGGCTAGCCAACAGGG
CCAA

In some embodiments, the intronic domain that promotes trans-splicing primarily in the heart comprises or consists of sequences from the gene TXLNGY. In some embodiments, the sequences from gene TXLNGY comprise or consist of:

(SEQ ID NO: 985)
AGAAGAAATTGTCCTGGATCATACAATAAGCTAGGATTTGCTCCA
GACCATTTTTTTCATTTTATCAGGCTACCTACAATTAAGGAATTT
CAGAAGATTTTGTGAACAAAAAATGTCCAGGAATTATGTGTGTAA
TAAATGAATTTGATATTTTGATCTCCTTATCCATCTTTTTCTTGA
TAAAATATCAGCTTTTTTTTTTCCAATGTAGGGCAGAAGCAAGAA
GTACAGATATTTTGATGGGGACAGCCCACGTCTGATAGGGTGGGA
CATGGGGCAGCAGTTAGAGGGTTGTGCTCTTTCTAGTGTGGGATA
GTTTGCAAGATGATATGTTGTAGCCACTTCTGCAGTGACGGGGAC
AGAAGGAGTCAGTGTTCTTAACTACAGTGGTAGCAGCAGAAAAGA
AGGGTAAGCAGGGTTCTAGAAATTTGTGTTATGTTTTCTCCCCAC
TGTATTTATTTCTTTGGTTAGTGGTGCAAGAAATTCTGTTTTCCT
GTAG

In some embodiments, the intronic domain that promotes trans-splicing primarily in the heart comprises or consists of sequences from the gene TXLNGY. In some embodiments, the sequences from gene TXLNGY comprise or consist of:

(SEQ ID NO: 986)
TAAGTTCTACTTCTTGGAAAAAAAAAAAAGATCGCCAAAATTGTA
ACCAAAGCCATTTAAAACAAACTCCTCACTTAACATAGTGCCAGG
GCATGCAATTTTGTTTCTTACTCTCTGGATGTGGGATATGCGAGT
GTGTGTGTGTGTGTGTGTGTGTGTGTATTAAGCTTTCTGTTTCTG
ATGAAATTTTCATATGAAATTTACTGGAGATAAGATTAAATTAGT
GGAAAAACAAGATAATTTTGCTTTCTATGTAGTGGTCACCAAGTT
AAAGAATTGTGCCAGTTATTAGGCAAGATAGGCTTTGAGATTTAG
GGACAGTTAAATTTTACACCAACTTAGTGAACATGAGACTTCTAC
CTAGTGTTACATTTCATTTTTAAATAAGCAATTTAAAAATTGGTA
AATGATTTGTTTACTTTGATTATATTCTGGTATAATTTTCTGACA
AAATTATCTGTGTCTTGGTCAGCATTGTTGCTAGAATATGTATTC
AGAT

In some embodiments, the intronic domain that promotes trans-splicing primarily in the heart comprises or consists of sequences from the gene MICU1. In some embodiments, the sequences from gene MICU1 comprise or consist of:

(SEQ ID NO: 987)
GTACTTTTTAAATTGATCTTTGAAGGCTTGGGCTCTTTAATGGTA
GTTTATAAATTATTAAAATTTATTCATTAAAATATTACTCTCACA
TTTCTGGTGGCTGGCAGTCTTAAAAATATAAATTAAACTGAGTTG
TAGTAATTAAATATAAATTAAGCTGAATTATAAACATCTTTTGGT
TTAAAATAACTAATATAACTAACGAAGACCACAGGGTGCTCTTTA
TTTTTACAGTACATTTTTTCTATACTTCCTTTGTTTGAAAAATGG
GGTGTAATTTTTGGTAGTCCCTCTTACCAAACCCTCAAATTAGGT
ATCTTATTATTAGTACAATGGATTATCTTCCTAAATACCTTTGTC
TTTAACTGCATTCTGAACCAAACCAACAAATACATTAAAAATTAT
GTCAGAATAACGTATAGACGATTCCATTATCAATGTTTCTAATCC
TGGGAATTCTATTTTAGAACAGCCATGGCACAAACATCATACAGA
GTAC

In some embodiments, the intronic domain that promotes trans-splicing primarily in the heart comprises or consists of sequences from the gene MICU1. In some embodiments, the sequences from gene MICU1 comprise or consist of:

(SEQ ID NO: 988)
AGGTTAAGAAATTTTGTTGTAATCACTCTTGTTAAGCATAAGAGA
TACTTAATTAATTGAGTCACTTAAATAGTCTTACTTGGAGGCCGG
GTCTGGTGGCTCATGCTTGTAATCCCAGCACTTTGAGAGGCAGAG
GCAGGAGGATCACTTGAGCAACCGGGAGTTCAAGACCAGCCTGAG
CAACATAGCAAGACCTCATCTCTATGAAAACATTTAAAAGCAGTA
GTAGTCCTAGCTACTCAGGAGACTGACGTGGGAAGATCGCTTGAT
CCCAGGAGTTGGAGGTTGTAGTGAGCTAAGATTGTGACACTACTC
CAGCCTGGGTGACAGAGTGAGACCCTGTCTCTAAAACAAAAACAA
AAAAGTCTTATTTGGGATATTATGTTCCTACTTGAACTGTAACAG
GACTGATGAGACAACAGTAACTTCAAGTTAAAAATTAATCTTTTT
TTCTTTTGAAATTTCTTACATAATAAATACAAATAAGCAGTTGCT
TGGT

In some embodiments, the intronic domain that promotes trans-splicing primarily in the heart comprises or consists of sequences from the gene VDAC2. In some embodiments, the sequences from gene VDAC2 comprise or consist of:

(SEQ ID NO: 989)
GGCGGCCAAGCCGGAGGCCCGCGCCGGACTCGGAGTCGGCCCTGG
CTTCCTAAGATGGCCGCGCTGCCCCGCGGCCCCTCGCCCCTCTCT
GCCCGGGATCTCCCTTTGGCCCTTTGACCCCAGCTTACCGCACTT
CTTGTCCCTCCCGCAGATTCCCCTCTTCCCGCGGCCTCGCCATGG
CGACCCACGGACAGACTTGCGCGCGTCGTAAGTAAAGCTGGGATC
TCTGCGGGATGGGGCGGCGGAGAGTCGAAGCCTGTCGACCAGAAA
CCCAGTTTGTTTCCCCCTATCTTTCCAAGAACTTGGAAATTCGGC
TGCTTTTGGTGGTCGCCCCACCGTCTGACCACCTCCTCTGCGGAG
GAGGGGCTGGGCTGCTGGATTTCAAATGGGGAACAAGGCCCGGGG
AGTTGGTCTGTGGCCGCATGGACTTTTCAGCCTTTGTACATGTCT
TTGACGTATAGAAGTAAAAAGTTGTTAATTGGGGATTCTGCCTTT
GGAG

In some embodiments, the intronic domain that promotes trans-splicing primarily in the heart comprises or consists of sequences from the gene VDAC2. In some embodiments, the sequences from gene VDAC2 comprise or consist of:

(SEQ ID NO: 990)
TAAGTTTGAAGCGCTATTTGATATTTAGTTGATGTAGACATTTGA
GTGAAACACACATGGCAGTATTTTTTCCAAGTTTCAATATTTAAA
TTCCCAGTATTAAATTCTCTTCCCACTCCAGGGTGTTGGTTTTCC
CCTCAGCGAAGATATTAAAGGACGTATCAGGGTAAAGGGCCTAGT
CTTAGGTTTCGGTTTTATCTAATTTAGTTAAATCAGCTCATCTCA
GTGAATTTTGGCAGATACATAAATATCCATACACACAAATACTTG
TTTGATTCTTTGTACTTGTCCAGTCCTAGGAAGTGAGGCTGTAGG
TAATAGACTGTTTTTCCGTTTTGAAACGAATTCCCCAAGTTTGTA
TAATCATGGTTATCTGTTTTAGAAATAATTCGTTGGTGGTATCTC
CTTTATGGGTCGTTATAGTTCTATTGTAGCTCCTGACCGCATAAA
AAACATTCCAACAAATGGGATTGCTTGAATTTTAATATCAGCAGT
GGGT

In some embodiments, the intronic domain that promotes trans-splicing primarily in the heart comprises or consists of sequences from the gene CYP2C8. In some embodiments, the sequences from gene CYP2C8 comprise or consist of:

(SEQ ID NO: 991)
GTAAGTCTGCCTTATGTTCCTCCAGCCAATTGCAAAGGGTAAGTT
ATTTGACTGCTATTTTTAGACAAAATATATTCCTGGAAGCACACT
ATTATAATAGATCATTGTAAAGCAAAATACTCCCTCTGAACTTCT
TTGATGTTTCTTTTGTCTTCCTATTTTTTTTTTTTTTGAGACGGA
GTCTCGCTCTATTGCCCAGGCTGGAGTGCAGTGGCACTATCTCCA
CTCACTGCAAGCTCTGCCTCCCAGGTTCACACCATTCTCCTGCCT
CAGCCTCCCCCGAGTAACTGGGACTACAGGTGCCCTCCACCATGC
CCGGCTAATTTTTTGTATGTTTTAGTAGAGACAGGGTTTCACCGT
GTTAGCCAGAATGGTCTCAGTCTCCTGACCTCATGATCTGCCTGC
CTTGGCCTCCCAAAGTGCTGGGATTACATGTGTGAGCCACCGTAC
CTGGCCTCTTTTGTCTTTCTAAGTCTGTCATTGTCAGAAATAGCG
GAGT

In some embodiments, the intronic domain that promotes trans-splicing primarily in the heart comprises or consists of sequences from the gene CYP2C8. In some embodiments, the sequences from gene CYP2C8 comprise or consist of:

(SEQ ID NO: 992)
TATACATTTATTAGCTAATAATTTGATATATGTATGGTAATTCAA
CATGTATGAGTTATATTCACTATTTCATGTTTAGGCAGCTGTATT
TTAAGTGAACTATACTAAATATTTGAAAGGCTTTTGTTATCAAGG
GCTAAGTCTCCTATTTTTTGATATAGCATTACAATGTACATTTTT
TATACACAAAATATAGAATACACTGATTTCCCTCAAGGTCATAAA
TTCCCAACTGGTCATTAATCTGAGAATATTGAATTTTGAGTATAT
TCTAACATAGAATCATTTACTTCAGTGTTTCTCCATCATCACAGC
ACATTGGAACAACCAGGGACTTTTAATTAAAAATACCTGGGCTCC
AATCCAATACAATTAAACCAGAATCTCCTAGATTGGCACTGGAAA
GAAGGAGTAGGACAAAAGAACATTTTATTTCTATCCATGGGCCAA
AGTCCACTCAGAAAAAAAGTATAAATTGGATCTAGGTGATTGTTT
ACTT

In some embodiments, the intronic domain that promotes trans-splicing primarily in the heart comprises or consists of sequences from the gene GPAM. In some embodiments, the sequences from gene GPAM comprise or consist of:

(SEQ ID NO: 993)
GTGAGTGAAGACCCTTCAGTGTTGCCTGGTCTCTTTTCTTGAGCG
TTTTCATGAAAACGTAACCCCCCGTGTTTTTCTGTTAGATAATGT
TTGACCCATTGTTGATACTTTATATTCCACTCCTGGATTTCAGTG
ACCACCAAACCAATATGTCATTTACTATCTTGGTTTGCTCTCTGG
GTCACTGCTCCACTGAATCTATCCTGCAGCTATGAGCCATGATAG
GACAGATTGCTCAATGTTCTCAGGCAGTCACATCCAAAGCTAGTG
AGCACGTATTGTGTGTGAGGCACTGGGTCAGTGGTTTGTAAATAA
TTAACACACCCACCTGTAGATGATGGAACCACAGATGGTAAGAGT
CAGAAGCAGCCTTAAAAATCATCTGCTCGGCCAGGTGCGGTGGCT
CACGCCTGTAATCTCAGCACTTTGGGAGGCTGAGGCGGGCAGATC
ACGAGGTCAGGAGATCGAGACCATCCTGGCTAACGTGGAGAAACC
CCGT

In some embodiments, the intronic domain that promotes trans-splicing primarily in the heart comprises or consists of sequences from the gene GPAM. In some embodiments, the sequences from gene GPAM comprise or consist of:

(SEQ ID NO: 994)
GCCTAAAACACATAGTGGTATCTGCTTTAATTTGGGTGATAGCCC
CTTGCTTGAGGGAATGTGGCTATAATTTTAGTTTTCAAACGAAAA
TGTAATCTCCTGTTATATCACTGTTTACAATGAGTAGTTACTGAA
TTACCGTTTGTATAGCATTACCCTTATTTTTCAGGAGAGGTAGAG
TGTCTTGCTATCCAGGAGCTGAAAATCTAACTGAGATGAGACATA
CACAGAGCAGCAAAGTGAGTGGAGGGCCTCATTTGTGATTAAGAG
AAAAGAGAGGAAGGAGAGAAGAGGGGGCTTATGTCTTTGGAGAAG
GTTTTTGATGCAGTTGGGATCATATGGAATCATAAGAATTTAGTC
TTATGGGATTTACTTAAATTTCTCCAAATACAGGCCATTATTTTT
GATGTATTAATAATCTCTTAAGGTTATTCAAGCCAGTTGAAGTTT
TTCTTGGAGACTTTGAGCTGGCAACTTCATTGTTGCTTTTCTTTT
CTTA

In some embodiments, the intronic domain that promotes trans-splicing primarily in the heart comprises or consists of sequences from the gene ACSL5. In some embodiments, the sequences from gene ACSL5 comprise or consist of:

(SEQ ID NO: 995)
GCTACCTATTAGCTACATAAAGTATCTTTCTCTCCACCTCTTATT
TCCTACAGGTGACCCCAAAGGAGCCATGATAACCCATCAAAATAT
TGTTTCAAATGCTGCTGCCTTTCTCAAATGTGTGGAGGTCAGTGG
TCAGTTGAAAAAGAGGCTCTCATTAAAATGTGAATCTGTCATAAG
ATTTTTATTTTTGAGGTTAGAGCAGGCAGACACACACACACACAT
ACACACACACACACACACACGTTAAAGGCTGGAGTGTGGACAAAC
ACCAGCGTGTTCTTTGTTTAGAGGACTGTACATTTTCAAGCGTTG
GTCTTTTAGGATCACTGCAAATTTCTACACTATATGCAGTTCAAG
GCATCTCAGATCTACACAGTGTACCGCCTATACAGCTGTCTGTGG
TAGTCCTGGGAGGAGGTAGTTTTAAAATCTCCATTGGAATTGAAA
GTTGGCATTAATCTCATGTTGCCTCCTCTTACTTCCCTGGCCTAC
ATAG

In some embodiments, the intronic domain that promotes trans-splicing primarily in the heart comprises or consists of sequences from the gene ACSL5. In some embodiments, the sequences from gene ACSL5 comprise or consist of:

(SEQ ID NO: 996)
TGAGTGTTCTGTGTTCCTAGCAGTATGTGGCAAGGGGGTCCTGAC
TTGCTATCACATGTTTATTCCAAAGGCAGCTACACAGTATTTACT
GTTCTTTCTCCGGTGTGAGAGGTGCAGGCAAGGAGTTGGCTCATG
GGAGCCCTAATCATGGCATGGCCCCTCATTTCCTGCAGAGTCTTC
CTCTGCTAAGTCTGCATGTGTCCAGGGAGGATAGTACTTGCTCCT
GCCTTCATGGGGGTGTGTGAGTCTAGTGACATCACAGCTAGGAGC
TTACTATCTATCTGCCCTTAATTCTGAGCTGACACAAGACATTGT
CTTCTTTTTAAATTTTATTTTGGTTAGAATTTTTGAAAAGATCTA
GGATTAGAGAAAAGGAAGTTCCCTACAGAGGATCAGGGAAGGAGC
ATTTGATCTACATAAGGTGTGAGATGTCCCCCGCGCCGGTTTTTG
CACACCTGAGTGTGTAAGGTGGTGCTGTCAGCATGGTGCATACCG
GAGG

In some embodiments, the intronic domain that promotes trans-splicing primarily in the heart comprises or consists of sequences from the gene HABP2. In some embodiments, the sequences from gene HABP2 comprise or consist of:

(SEQ ID NO: 997)
TGAGGTTTAAAACAAAACTTTTGAAGCCAACAAGCAACTGTGGGC
AACACTTCAAAGGGCCCAGAATCAATGTTTGTCAATAGCAGTTTT
GCATCCTTTAATTTATGTCTCATGAATATAAAGTGTGATGTTATT
AGGAGCCCATCCATGCCTTAATTCTGTTTGAGATTTGATGAACCA
GAAGTCTATTAACATTCTGTACAAATCAGCACTGGCAGAGGCCTG
TGGCACAGGCTTCACCTGATCTTTTATCCTGAGGCTGGGATGCTA
GTCACGCTGTCAGGCTTCCTGCTCGGAACACCCAGCAGCTGTCAT
GTGCACCGTGGAGCCAGGGGGCTATGTCAGCTACTTTCTTTGGAG
AGTTTCTCCCCAGAAGAGAAACCACTTCATGACTCAGCCCAGCCC
CAAAGTTGCCATTTGTTTAATCAAGATAGTTAGTTTGCAAAACAT
TAAACATTAAACAGTATTTGATGCCTAGTTAATAATTTGCATCCT
CTTC

In some embodiments, the intronic domain that promotes trans-splicing primarily in the heart comprises or consists of sequences from the gene HABP2. In some embodiments, the sequences from gene HABP2 comprise or consist of:

(SEQ ID NO: 998)
TAAGTGTTCTTTTCTAATATTTGTAGTTGAGAGACTCCGAAGTTT
ACTAGGAGGACCAAGCTAGGCTGGGAGTGATGAGAAACTTTAGAG
TGAAAGGCTGGAGTTGCCAAGTTGGAGATGTTCTAACTACAAGAA
CCAATGCAGGCCTGTGGCTTGGTTTCTTTTGAGCAGCCACTCCCA
TGGGAACAGGTGTCTGACTTGAGCCATCTTGGAGCGGAGCCTCCA
TGAATGCATTCCAGCAAAGGAGACTGGGGCAAGAAAATGTGCCCC
AGGGCATGGATTGTAATCTGTCATGGATCTCATAGACCCCTTTGA
GAAGCCATGGAAAGCTATAAACTCTCTCCCCAGGAAAATATACCT
ATCTATTGGTGCTGACACACACAGTTTTCCAGCGATTTGTGGCTG
ACACTAGGGTAAGAAGCAATGTGCTATGGCCTTAGAGGGGAAGCC
TGCTGACTGCAAAAGATCCTGGCAGTGGGTCTGCATAAATAGTTT
GGAT

In some embodiments, the intronic domain that promotes trans-splicing primarily in the heart comprises or consists of sequences from the gene IGF2. In some embodiments, the sequences from gene IGF2 comprise or consist of:

(SEQ ID NO: 999)
GTAGGATTTGAGCTGTGTTTCCCGCCCTGATCCTCTCTCCTCTGG
CGGCCGGAGCCTCCGTAGGCTCCAAGCCTGGCCCAGATTCGGCCC
GGCGCAGCCGGCCTTCCGCGCGTCCCGCACCTGGCGGGGGCTCCG
GGGCTCCGGCGCGGCACCGGGGGGCGCTCGGGATCTGGCTGAGGC
TCCAAGCGCCGCGTGGCCGGCTCCTCCTGCTGGGGCAGGTGGCGG
CTGCGCGCCCCGCCCGAGCCCAGGGGCCCCCTCAGCCGCAACAAC
CAGCAAGGACCCCCCGACTCAGCCCCAAGCCACCTGCATCTGCAC
TCAGACGGGGCGCACCCGCAGTGCAGCCTCCTGGTGGGGCGCTGG
GAGCCCGCCTGCCCCTGCCTGCCCGGAGACCCCAGCTCACGAGCA
CAGGCCGCCCGGGCACCCCAGAAACCCGGGATGGGGCCCCTGAAT
TCTCTAGAACGGGCATTCAGCATGGCCTTGGCGCTCTGCGGCTCC
CTGC

In some embodiments, the intronic domain that promotes trans-splicing primarily in the heart comprises or consists of sequences from the gene IGF2. In some embodiments, the sequences from gene IGF2 comprise or consist of:

(SEQ ID NO: 1000)
CCGCATCTGCTGCTGTCCCGCCGGTGCTGGCGCTCGTCTCCGGCT
GCCGCCGGGGAGGCCGGCGTGGGGCGCGGGACACGGCTGCGGACT
TGCGGCTGGCGGCTGCGCTCGCTCCTGCTGGGCGCCCCGAAATCC
GCGCCACTTTCGTTTGCTCATTGCAAAGATCTCATTTGTGGGGAA
AGCGGCTGGAGGGTCCCAAAGTGGGGCGGGCAGGGGGCTGGGGCG
AGGGACGCGGAGGAGAGGCGCTCCCGCCGGGCGGTAAAGTGCCTC
TAGCCCGCGGGCCTAGGACTCCGCCGGGAGGCGCGCGCGGAGCGC
GGGCGAAGTGATTGATGGCGGAGCGAGGGGGGCGAGGGGGGCCAG
GGGGGCGCGAGATTCCGCCGGCGGCCCCTTCCCCTTGGCTAGGCT
TAGGCGGCGGGGGGCTGGCGGGGTGCGGGATTTTGTGCGTGGTTT
TTGACTTGGTAAAAATCACAGTGCTTTCTTACATCGTTCAAACTC
TCCA

In some embodiments, the intronic domain that promotes trans-splicing primarily in the heart comprises or consists of sequences from the gene PSMA1. In some embodiments, the sequences from gene PSMA1 comprise or consist of:

(SEQ ID NO: 1001)
GTGAGAATATCTTTGCTTTTTGATAAAACACAGAATGCATAGATT
TCACATCTCACTATGTGAGCCCCCACAAAACAAAACCTAGAGGAG
CATGTGAGAAAAACGCATCTCTAGGCTAGAAAACAAAAAGAGAAA
AAAAGAAAAATGTCTGTGTAAGAAAGCAAAGGAAAGAAGATGCTC
AAATCCAGCAAATCATACCAGCCTTGTCATAGGCACAAAAAGAAA
GGGATTAACCTAAGTTACAAGGTGAATTATCATAAAATCAGATGA
ACCTTTCTGAAAAATAGTCATGGGACAAAGGAGGGATATATAACA
CTAGAGGCTTAACCCTTTCCTGCAGAGAAAAAAAATGTAACTGAA
AGAAATGCCACTCAAGGCTTGTGACATTCAAAGTGAAGACAAGAT
AAGGTCTTATGTAGTACATGACATAGGTGTAGACACTCACTAGAC
TGAAAGGAGTACATGTTTCCAAAGCAGCTTTAATTCACGTCTCTG
CTGA

In some embodiments, the intronic domain that promotes trans-splicing primarily in the heart comprises or consists of sequences from the gene PSMA1. In some embodiments, the sequences from gene PSMA1 comprise or consist of:

(SEQ ID NO: 1002)
TGACCTAATTGTATTCATTTGTCAAGAGGCCGGTTCCATATCGGA
GACAGGCTGTATTCATAGATGATTTTAAGGACAGTTTAAGGCATA
GAGTTTTACTCTAAAAACTGAGAAACAAAAAATCCTTCATTGTAG
AATTTGTTTTGTTGCCCTAGCTCTACTATTTATACTTTTCCACTT
TAGCTATGCACAGAACATAGCTAATTTTCATCTCAATTTTTCAAT
TCTTTTGCTATATAATTGTTAACATTTATTGACCACTAACCTCAT
TTTAAGGACTGTGCTAAGTATTTTATATACTTTCCTGGGGAGGCA
CTTATTCCTCACTTTTGATATTTCCCTCCACGTGGGGAACATCCA
TTAGAGTAACTCTCCTGGCCTGTATCTACTTACCCCCAGGAGAGG
AGAAGAGAGTTTGGTGAGTTTGAGATGAGGAACAGGATGTCTGAT
GAAGATCTAATATAACTCATGTTCCTTTTACCTTCTCCATTGTCT
TTCA

In some embodiments, the intronic domain that promotes trans-splicing primarily in the heart comprises or consists of sequences from the gene DEPDC7. In some embodiments, the sequences from gene DEPDC7 comprise or consist of:

(SEQ ID NO: 1003)
TGCCTTTATGGTATTCACCATTCATTGTTAATTCCTAGTCTTGCT
TTTGCTTTTGGAAATAATTTTGAATAAGTGCACTTTTTAACTCAA
ACTGTAACTTGAGTTTTGTTCCCTTTAATATAGTCATATTTGGTC
AATTAAAAGCAGTTTTTTTCTTTTGGCTAAAGTGAATGGGAAGAC
GGAAATAGCTTTAGAAGCTACCCAGCTCCTTCTAAAGCTTTTAGA
TTTCCAAAATAGAGAAGAATTTAGAAGACTACTGTATTTCATGGC
TGTTGCAGCAAATCCTTCTGAGTTTAAATTACAGAAAGAAGTAAG
TCTTTTGTTTAACTGTTAGTTGTATTTAATATCCTTAATACTTAC
AGGTTAAGTATTAGATTATTCCATCTTCAGCATCAAGTATGCTGA
AATGTATAATAGCTATTAATATTTAATATTCCCTTTTATATTAAA
CTTGTCTCTTAAATATATACTATTGGATATATTTGTTTTATTTTT
ATAG

In some embodiments, the intronic domain that promotes trans-splicing primarily in the heart comprises or consists of sequences from the gene DEPDC7. In some embodiments, the sequences from gene DEPDC7 comprise or consist of:

(SEQ ID NO: 1004)
TAAAATTGTGAAAGTAGTTAAATTGAGCTTATGTAATAGATCACT
AGATTAAAATGCATTTGAATAATGTCCCATGTCCCTTCTTTTTCT
TAAGATTCCTGGAACTCTACATAAAATTGTAAGTGTTAAGCTTAT
GGCCATACAGAACGGAAGAGATCCAAATAGAGATGCAGGTAATCT
GAGAAATATTATTTTCATGATCTTCCAAAGACAAATTTCAGGTGT
TTTTTGAAAATGTTTTGCTATAAGTAGTTGGACTTTAATTTCAAC
CAAATTTAAAACATTCTCCTCAGAGTTATTCATACAAAGCAACTT
TACAAGTGTTAGAAAGTCAGCAGTGCTTACCATCTTCAGTGCATA
TTACAAAATGGGGAAGAAAAACATAAAGACAAGAATATTGCTTGA
TTTTTCTGCTTAAATATGAGGAATTGAGTCATTAACTGAACTTTT
TACTTTTAAACTTTAAGGATATATTTATTGCCAGAGAATTGATCA
ACGT

In some embodiments, the intronic domain that promotes trans-splicing primarily in the heart comprises or consists of sequences from the gene CTNND1. In some embodiments, the sequences from gene CTNND1 comprise or consist of:

(SEQ ID NO: 1005)
TGAGAGAGGAGACCACAATAGAACACTGGATCGATCGGGGGATCT
AGGCGACATGGAGCCATTGAAGGGAACAACACCCTTGATGGTAAA
TTCTCTTTATATACTGCTATCTGTCTAAGGCTAGTTCTATTTTGA
AGCCTATGTGTTTTGTGAAACACAAAAAAAAGTACAGAGAAAGAT
CGCATCCTTTTCTGGTAGGGGTTTTCAGGAAAAAGTAAGAGTTCT
GACTCATGTTGGGATTTCTTGGGCCGTTATTCTGCAGTGGTCAAA
ATGGGGGAAGCATGTCTGTAAAAGTGTTACTGATATGACTAACAC
TAACTGATCTACTTTCAAACATTACCTTTTTCCTCTCCCTCCCTG
TTTATGAATGTTTTGCCCTTCTCTTACTATTATATCTTGTTTTGT
TTGAGTTTACAAATTTTCAGAGTTTCCCTAGCTCTGGCACACACT
CATGAGGTTCCTGTCTCTACTCATACTAACAAATTGCATGTGTTC
ACAG

In some embodiments, the intronic domain that promotes trans-splicing primarily in the heart comprises or consists of sequences from the gene CTNND1. In some embodiments, the sequences from gene CTNND1 comprise or consist of:

(SEQ ID NO: 1006)
TATGTCCTTCAGTCACCCCCAAGATTGTTCTTGAGGAAGGAGGCT
CCTGAGTGCCAGCAACTTGTCAATCACGCTAATCTGATCAGGCCA
GAGACTAAGTAGTCTCAGCCACATGGGGCTCGAGGGGTTCTGCTT
TTGTGATTATTACCTCTTACGAGTCTGGGACATGCAGCTGGTGAT
CTGATTCTCCGTTATGTGCTGCTTTCCTTTCTCCAGATTTGCTCA
ACTTTTTTGGGGGGGGTCTCCTTAATCCCAACCCCATTAACATTC
TCTCTTTCTCTTTTTTCTCCACAGCAGAAGATTTAGCACCACTAT
CTCCGTTCCATCTGGGCTTATATGTACTTTTATTTTTTGGTGGTG
AAATTGACTGATGATTTTCCTTTTTCTTCGCTGGACTATTGTGCC
AACTGCCAGGCTGCCTCCTGCCCTTACAGCCCTAAGTGGCTGCCT
TCTTTCCATCAACTCCCAACTTCTTCCTGTGAAGTTTAATTGTCT
CAAC

In some embodiments, the intronic domain that promotes trans-splicing primarily in the heart comprises or consists of sequences from the gene MS4A6A. In some embodiments, the sequences from gene MS4A6A comprise or consist of:

(SEQ ID NO: 1007)
GTGAGCAAGGTTGGAGGTGATTCTGCCCCTCTCCCCAGGCTTTCT
GTGTGAGTCCATTCCTCCTCTCAGATTTATTGTTAGAACTTAAGA
CAAGCCAATTACATTTCATAATGTCTGTGTCATTCAGACGCTGAG
AACTAATCCAGCCTTCTAAATGGTGCTTCTTAACTCCTTTCTTCA
ACAGGAGAGTTAATGTGTGGAATTAAGTGCAGAATCCATGGCCCT
CTGTGGTGACGGTGATGGTTCAGGTTGTGTTTCTGGGTTCATTCT
GGAAGCTCCCCCAAGGAAAGGAGGAAGGAAGCTTGCAGGGTGGGG
CTTTGCCATTGCCCTGACTGGCTCTGGTTTCCTTGCCTGATACAT
TGAAGTCAGCTTCCAAAGAATGCCACCCAAGGTTTTGAAGGGGCA
CAGTGCCTCTGTCCTCACAAAACCAGCCTGCATATGAGTTTACTG
AATTCATTTAGGTCCTGCTGAATCTTTCCCATTTGTTCCTTCTCT
GTTT

In some embodiments, the intronic domain that promotes trans-splicing primarily in the heart comprises or consists of sequences from the gene MS4A6A. In some embodiments, the sequences from gene MS4A6A comprise or consist of:

(SEQ ID NO: 1008)
TTCTGGTTGTTACACATAGGGTGGGATTGTGCTGGCATAGGCCAG
GGAGTCTGCTAAGCATCCTACAGTGCACAGGATAGCCACCTACTG
TAAAGAATTACCCAGTCCAAAGTGGCATTAGTAGCTTTTTTGAGA
AACCTGTCCCAGATCATTAATCAGAACGTGTTATTGATATACCAG
TCTTCTCCCTTGTAGTTAGGTTGGTAGAATTCAGACTCTGGAAAA
AACTGGGAATGTAAAAGGCTGAATGAGTGTAAGCTGAAAGACAAG
AGAAAGGAGAATTGTTTTAAGCTTACTAAAGCCAAAATGGAAAAA
AAAAGAGGGGGGAGGATTGGGGGGTGAGGAAGACAGTCAACAGAC
CGCTTTTAGGCCATCATTCCGAAGGGTTCTGTGCTAGGAACCTTA
CACGTGTTGTCTCACATAATCCTTAAAACTCAGAAATGATTTTAA
ATTAAATAGCACAGAGAGGAAAACTGACTGGAGAGGGAGCCTTTG
CTTA

In some embodiments, the intronic domain that promotes trans-splicing primarily in the heart comprises or consists of sequences from the gene SLCO2B1. In some embodiments, the sequences from gene SLCO2B1 comprise or consist of:

(SEQ ID NO: 1009)
AAAATGGGGCTAATGTACACCTCGCTCATAGGAGTGATGTGGGGT
TGCAGGGCAGAGTTACTGAGCCCGTAGTAAGCGCTCCATAAATGC
CAGCCACTGGTGTTGTCTGGCAGGAGCTGGCGTTACAGTGGTTGG
GGATGCTGGGGAAGTGTCTGACTGTCCCAGCTCCAGGTCTTGCCA
ACTGTAGGAGTAAGGGGAGAATTTGATGGGACCAGTGGGTGGAGG
AAGAGGAGTGATGTCTGTGACCAGAAACATACAGAGGTCAGCACA
GGACCCTGATGGTCAGTGGGTGTGTTGTCTTGTATATGGGACCAG
GCTCTGCAGGTTGAAAAAGGATGGGGTACGTCCCTGAGCCCCCAG
CCTAGATCATCAGATGGGAGAGAGGCTGGCTCTGGGTGGTGGGAG
AGAAGGGCATAATAAGAACCCTTCAACGTTGATGCATGGCCCTTG
GGGGCTGGAACCCCTCCAACCTCATGTTGCCCATGTCTCTGCTTG
TTAG

In some embodiments, the intronic domain that promotes trans-splicing primarily in the heart comprises or consists of sequences from the gene SLCO2B1. In some embodiments, the sequences from gene SLCO2B1 comprise or consist of:

(SEQ ID NO: 1010)
TAAAGATCTTGCTTGGGACCATTGGTGGTGGTGATGGGGTCCAGA
TGCCCACTGTGTGCCAGGCCTGGGGCACAGGCATGAGCGGAATTC
AATCCCTGATCTTAGGGCTCACAAGCTCATGGGGTGACAGACCTG
GCCCAGCTCTCCTAGAGCGGGGTATAGAGGCAGAGTCTAGACAGC
CCGTCAGGAAGCCACCTCGGATGCAGGGGTGGGGCGGGCTTGAGG
GCCAGACAGCCAAGAGGCCCCAGTAACCCAGAGCCCCTGAGGGGT
TGTACATGCCAGGGAGGGGGCAGGGTAGGACGGTGGGCCTTCATT
GTCCCCTGAGCACCACCTCCCTCAGCCTGGATGCCCAGCCCCGTG
ATCCACGGCAGCGCCATCGACACCACCTGTGTGCACTGGGCCCTG
AGCTGTGGGCGTCGAGCTGTCTGTCGCTACTACAATAATGACCTG
CTCCGAAACCGGTGAGACCTGGTTTGATGGCTTGTGGGAAGGGTG
CTGG

In some embodiments, the intronic domain that promotes trans-splicing primarily in the heart comprises or consists of sequences from the gene PICALM. In some embodiments, the sequences from gene PICALM comprise or consist of:

(SEQ ID NO: 1011)
GTAAGTAAATAAGGAGAGCTTCAATAGTTCCATGGTAGAGAGAGG
CCTGCTGGAAGGTTCAGAAAATAGGCCGTTTGGAACCTTCTCCTT
TAAGCCAGGAGGGCGGCTTTGGGAGGCGTCTTGAAATACATTCTT
GACTCTGCTGGGAAAGCTCGGGGAGCCAGAGGCCTAATACCCCCC
ACTTCAGGGGAAGCTGTGAAGGCAGCTGCACCTCCTTTGTCAAGC
TGTTGGTTTCAACCACAGACACGAGGACACACTTGAGGTGGGGGG
ATAAGGGGAGAAGCCTGCCGCCCCGCCCCACCCCCGCCAGCACAC
CGCCCTCGCCATTCCTCTCCCGCCCCGCCCCGCCCGGCGTCTGTG
ACCCCGCGGGCCGAGCCTCAGGCGACCTGTTGGCAGGGAGAGTCA
CGTGATGCGCCGGCTGTGCACTCCCCTCGGGCGGGCCTCCTGTTG
CGCCCGCCTGGCGAGTTCCATCACGTAACGCGCGGGCGCCGCCCA
GCTC

In some embodiments, the intronic domain that promotes trans-splicing primarily in the heart comprises or consists of sequences from the gene PICALM. In some embodiments, the sequences from gene PICALM comprise or consist of:

(SEQ ID NO: 1012)
TATTTTGGATTTGGCTGGCAGATGGTGTACACAGACTTTCTGAGA
ATGTTAGCATTTCATTTGAAACTAGCAAGATTACATGTGACTATC
ATGGTAACTTAGCACTCACATATTTTTCAACTTATCCCTAACCCT
GGGTTGAATGTAGAGGCTCTAATAACATATAAAAGGCTTTGTTAA
TGTATGGTCTGGGTCTCATTGAATAGTATTTCAGGATGTAATACT
TCTTTTAAAATGTCCACAGGGCAGTGTCAACGTCTTTCCATGGAA
ATCTGGCTCACCCAATAAGAGAAAAGAGGCCAAAGGTAAGAAAGA
CTAAACCAGGGGAGATGGCAGGCAGTGATGTCTGGACTGTTTGAA
TGGGACTTCTCCATTTACCGCCCCTCTCACTTCCTTCTATGGTCT
GGACCTTTAATCACCACAGCAGTCAGCCATGTAGGGTCAAGCTAA
TCGCTGTTGACATCATCCTACTACTTCCCTTTTCAAATTCCAGTG
AAGC

In some embodiments, the intronic domain that promotes trans-splicing primarily in the heart comprises or consists of sequences from the gene SLCO1B3. In some embodiments, the sequences from gene SLCO1B3 comprise or consist of:

(SEQ ID NO: 1013)
TTTAGTATGTGACTTGCAAATTTTTCTTGACAGCCAGACAAAATG
TATTGGGTAAAAGGAATTGCTGTATATAAGCCTTTATTAATGTGG
TGTAAGGCGATGAAGACAAAGTAGCATCCTGTAGTTCTATGATAA
GGCCTCAATCTTTTAGTGAGCCTGTACCTCTGGACTGTGAACTTC
AAACTTGCTTCTCAGTTTCTGTCTCCCACTCCTTAGGTAGAATGA
GATGGCTTGAGGGGGCTGGAGTTATGTATTTTCCTCCCTCCCAAA
GGAAGGCTAGAGACAGCTGGGGTTTAGTTTCTGTAGCTTCATATG
TTTTTTTAATAATAGAAAAATATCTTATGCCCCCAATGAAAATAA
TAAATAGAATTGTTCTCTTAATATTTGTATGTGTAACTTGTTTTT
CTTTCTTTTAAGATATGCATACTGGGGAGAAAAAAATGTAAGATA
TTTTACACATTTAAAATAATAATCGACTCTCTATTTTCTCTTTTC
ACAG

In some embodiments, the intronic domain that promotes trans-splicing primarily in the heart comprises or consists of sequences from the gene SLCO1B3. In some embodiments, the sequences from gene SLCO1B3 comprise or consist of:

(SEQ ID NO: 1014)
ACCCATTGATAAGATGTGTCTTCATTGCATCTGTCTGTTCTCTGG
GAGAAGCAAAATCTGTGTTACTTCCTTGCATACTTGTTTGTTTAT
TCATTGTGCACAAGAAGATATCTTCCACATCTTCACAAGCTGGAT
AAGAAATAGAGAAAATGAGATCAAATATTTAAATATTGCATTTTC
TGGTAGTGATGCAGGATATTTCTTGGCCTCTTCACTGGACTCACA
ATAGAGGTGCCCTGTTTATTTGGTCTGCCATGCTCAACCCCTTGT
GGGAGGGAGAGTGTGAGTGACGTAGTGCAGGACCATGTGGGCTGT
CCTGGTCATCGACACAGAAACAAGCTCTGTGAGTTTCTCATGGCC
AGACCAGGTGCGAGTGAGTGAATGCTGGACCTGGGCAACCGCTCC
AGGCACTGAAACGAGTGAGTTACATGCAGGGCTGGCAGCCAGGCA
AGGTGCAAGGGTGCTCAGGATCCTGAAGCCCCAGAGGGGTTGTTA
TGGT

In some embodiments, the intronic domain that promotes trans-splicing primarily in the heart comprises or consists of sequences from the gene ANO6. In some embodiments, the sequences from gene ANO6 comprise or consist of:

(SEQ ID NO: 1015)
AATTACTGATGCCTGAGGCCACCTCCAGAGATCTGGTTTAATTAT
TGGAATGCAGCCTCCACTGGGATTTCTAAAAGCTCCCAGAAGATG
TTATTGTGCACCAGGGGTAGTGAACCACAGACCTGAGATGTCAGG
GTCCTGAGAGCTCATCTCATCCAGTGGTTCTCAGCCCTGGCTGCA
GATTAGCATCATCTGGAGAGCTCATGAAAGAAGCACTGAGGTCCC
ACCACCTGAAACGACCTTTCAGAGGAAGGAGCCATTCTTTTTAGA
AGGTGCTCAGGTGATTTTGATGCACAGCAAGAGTTAAGGTTAACT
GACATCTTAGCCATCCCATGTTATAGATAAGGAAAATGAGAGCAA
AAATGGTCAAATGGCTTGTACGAGGTCAGGTCCTTGTTGTTACAG
AGGCAGGTCTGATGCCGCCTCCTGCTCTGTCTGCAGCCTAACACC
ATCCCTTATATTGGCCTGTTGTATTTATTGATGCCTCCTCATCCT
TCAG

In some embodiments, the intronic domain that promotes trans-splicing primarily in the heart comprises or consists of sequences from the gene ANO6. In some embodiments, the sequences from gene ANO6 comprise or consist of:

(SEQ ID NO: 1016)
TAAGATCCCCTTCCTTTGTATTTGAACAGCATCCTTCCTTCTTTT
TCCCCTCCGACTGTCAGTTCCGGGAGAACAGGCCTGTCTTTCCTG
TTATTTCTGTAACAGTAGTGGGGTTTCAGATAATATTTGTTGAAT
TGAAGTTCTGTAACTTTTTATTCCTCAGTCCACATGGATTGCTGA
AAATAATCCATTCCTATATGCATGCTTCAGAAAAGAACTTCAGAA
AGCAAAAGTTATTTCTCACTGCAACCTCTGCTTCCCAAGTAGCTG
GAATTACAGGTGCATGCCACCACACCCAGCTAATTTTTGTATTTT
TAGTAGAGACAGGGTTTTGCTATGTTGACCTGGCTGGTCTTGAAC
TCCTGACCTCAAGTGATCCACCCACCTCGGCCTCCCAAAGTGTGG
GGATTACAGGTGTGGGCCACCATGCCCGGCTGCAAA

In some embodiments, the intronic domain that promotes trans-splicing primarily in the heart comprises or consists of sequences from the gene KRT8. In some embodiments, the sequences from gene KRT8 comprise or consist of:

(SEQ ID NO: 1017)
GTGGGTGTGGGTACCTCTGACCGGACCTGCTTCCCTATCCCTGGG
ACCTGGGGTGGGGACGGTGGGAGCCCCCTGAAGCCCCTTGGGACT
TGGGGTCCTGTTGTTCTGGGCCAAGAAGGGCTAGGAGTTGGTCCT
GACACCCCATTTGTCTGGGTACAGGCTGGAGTCTGGGATGCAGAA
CATGAGTATTCATACGAAGACCACCAGCGGCTATGCAGGTGGGTG
TCCCAGGGCCCTGGGATGAGGGCGGGAGGCAGGGCCCAGGGAGGC
CTCAGCTCCAGGGAGGGGGCTGTGCTCAGTCGCTCACAGTGACCT
CAGCCTGAGCACTCATGTTCTTGGGAGAATCCTAGGGTGGGGAGG
CACATATTCAGGACCCCAGTAATAACTTTATTACTTAGTAACTTT
ACTATTAGAAAGCACCCAAATAAACCATAGCTGTGTGCCAGGCAC
TGTGCTAAGTATCCTACAGGTTTTATGTGATTTATTTTATTTATT
AATT

In some embodiments, the intronic domain that promotes trans-splicing primarily in the heart comprises or consists of sequences from the gene KRT8. In some embodiments, the sequences from gene KRT8 comprise or consist of:

(SEQ ID NO: 1018)
AAGGGTGTGGAGGGGAAGCACCTGGAAACTGCTCATGTCTCCCTG
ATCTGCTTCCTTAGTCTCGTTTATTTATTTATTTATTTTTGAGAC
AGAGTCTTGCTCTGTCGCCCAGGCTGGAGTGCAGTGGCGTGATCT
CGGCTCACTGCAAGCTCCGCCTCCTGGGTTCACACTATTCTCCTG
ACTCAGCCTCCTGAGTAGCTGGGACTACAGGCGCCCGCCACCAGG
CTGGCTAATTTTTTTTGTATTTTTACTAGAGACGGGGTTTCACTG
TGTTAGCCAGGATGGCCTCGATCTCCTGACCTTGTGATCTGCCCG
CCTCGGCCTCCCAAAGTGCTGGGATTACAGGCATGAGCCACTGTG
CCCGGCCCTTAGTCTCATTTAATTTGAGCTGGGGGGAGTCAGCCT
AGGTGGTGGTGGGGAGGACCCTGAGGGAGGGTGGGAGCACGGGAG
AAGAGAAGGCATACCCAACCTGACCTACTTACCTGTCCCCTACCC
CACA

In some embodiments, the intronic domain that promotes trans-splicing primarily in the heart comprises or consists of sequences from the gene CSAD. In some embodiments, the sequences from gene CSAD comprise or consist of:

(SEQ ID NO: 1019)
GTGCATACAGTCCCCTTCTTCCCAAATCCCGCCCTTCAAACCATT
GGTCCAGAGAATCATCATGGACTAGGGAGTTTAACATGGCTGGGG
TAGGAGGCTGAGGGGAGGGGGACTGGAGAGAGGATTGATTGGAGC
TGGGAGGTCAGACATGGCAGTTCTTCCTGATGCTGGGGTGAGGGT
GAGACCAAAGCCCCTTTGCTGGGGACCTGGAAAAGGAGGAAGGGC
TCTGGTTGGGCAGCTGGAGGAGGAGCCACTGACCTGCCTTTGCTC
CACCCCATCCAGGGCTGACTCTGTGGCCTGGAATCCCCACAAGCT
CCTCGCAGCAGGCCTGCAATGCTCTGCACTTCTTCTCCAGGATAC
CTCGGTGGGTCTGCCCCTGCCCCTGCACCAGCCCCACCTCTCTCT
GGAGCTCTGCCTTCTCTATCCCACCTTAGATAAAGTTCAGGCCTT
TCTCTATTCTCTTTACCATCCCTTCTGAAGCTGCATCTCAGCTAC
CACC

In some embodiments, the intronic domain that promotes trans-splicing primarily in the heart comprises or consists of sequences from the gene CSAD. In some embodiments, the sequences from gene CSAD comprise or consist of:

(SEQ ID NO: 1020)
CACTATGCCTGACTCAATTTTTTTTCAATAGCATTGGATCTTCCA
GTAAAACATTCAAGTTCTTTGAGAGTATGAGCTTTGGAAATGAAA
ACCTAGGTTCGAAGTCCATCTCTGCAGCTTCTGCTTGGATCTTTG
AATCTCAGTGTCCCTGGCTGCACATTAAAAAGGACAGTAACACAT
TGTTGTGAGGTTGAAATGGGCCATTTTAGGCATGCAAAGAGCTTA
GAGCTGGGCCCAGCATCAGGCTCTGAGTAGCTTTTGAGATGACTT
TACATCTTTCTCCCTCTACATTTTGGCAACAGGGTGCTGTGCCGT
TCCTGGTCAGTGCCACCTCTGGCACCACTGTGCTAGGGGCCTTTG
ACCCCCTGGAGGCAATTGCTGATGTGTGCCAGCGTCATGGGCTAT
GGCTGCATGTGGATGTGAGTGGGACTTCGGCTTGGGGGTGCGGTG
GGGTGGGCTGAGCCAAGGCCAAGGCCCACGTTGTTCCTCCTGCTC
CACA

In some embodiments, the intronic domain that promotes trans-splicing primarily in the heart comprises or consists of sequences from the gene RDH16. In some embodiments, the sequences from gene RDH16 comprise or consist of:

(SEQ ID NO: 1021)
GTAACCACCCAGGTTTGTCAAGTCTGTCTGTGTCTTCCCTCTGTG
TGAAGGGACCACCTCCTGTCTGCTCCCTGGGAAGATTCCTACAGT
GTCTTTCCAGGTTGAAGTAACAGTAGCTTTCTAATATTTCTCACT
TAACAGGCTGCCTGAACCCCATCTCTTTTCAATCCTTAACCTCTC
CCGACATTGGAGGTGTCTAGTCTTGGGGAGAGTTTGCCCTGATGA
GATTTTCTCTCCAGAATTCCTCTCTATCCTCTCCCTCCTGTCTAA
GCAGAAGTCATGTGTTGTTTGATGCTAAGAGCCTGGGTTAGAGAT
CTGGAGACTTGCTGGAGCATCGTTGAGAAGGGAGCACAGGGCTGC
AAGTGTGCACGGAGGCTGTGCCTGTGCTGGAGCCAGGCTTATGTT
CCCAGCACTTTACCTTGTTTTTGCTAGGGCCCATCAGCGTCCACT
GAGAGCTACGATCATCTCCATCTTCAGAGGAGAGTATCAGCTTAA
GGAC

In some embodiments, the intronic domain that promotes trans-splicing primarily in the heart comprises or consists of sequences from the gene RDH16. In some embodiments, the sequences from gene RDH16 comprise or consist of:

(SEQ ID NO: 1022)
TTCTCATCCTAGACAGCCTGACCGTGCCCTAGTTTTCAGAGGAGT
GATTGATTCTGGGGTGCGGAGCAGGATGAGGAAACTCAGGCTCAG
AGAGGGATTTCCACCTAATGTGCCTGGAAACCATGGGTAGCTACC
CAATAAGGCCTTTCTATGTTCAGAAGGTCATGGACCAGGAGTGTT
GGTATCGGAGCTTTGTAGAGACAGTTTTGACCCCTGTACTTAGAG
CAGAGGGGTGTGTTTGCCATGGAGAGTGTGTGTGGGCAGCTGTGT
GTAAGCCTGTGTGAGTTCTGTGTATGTTCCTGTGTGAATATATGT
GTGTGCTCCTTCATGGACACAGCATGCATGTAGTGGGCATAGCAA
GGCAGAGAGAAATCAGGAGACTAGGGGCACATTGCCCAGTAGGAA
GATAGTATCTTCCTACTGGGCAGGACTTGAGGTGTGCAAAGGTGT
GCATGAGTACTGCAAAAGCAGTTCAAAGAGCAGAGAGGTGGGGCC
AGAA

In some embodiments, the intronic domain that promotes trans-splicing primarily in the heart comprises or consists of sequences from the gene MON2. In some embodiments, the sequences from gene MON2 comprise or consist of:

(SEQ ID NO: 1023)
AAGCAGTCTACCTTACTTTTTTTTTTTTACTATATTGTTCTGAAG
TCAGGTTATTGCAAGAATAATCCATATTTCATATATTTTTAATGT
AGAAGATAATTGACTTATTTGGGGACAGGTGGAAAAGGGCTTTAT
CATCTATTCGTACTGATTTACTTTGCAGATACAAACTGTATAAAT
AACTTTGAATCATTTTTACCATTCTTTCTCATTTGTTTGCCCTTT
CTGTTGTTCATTCCTGTCCAGTCCATTCTTTACATGATAGCCGGA
AATGATTTTTCCATAGTAGGGCTTTGATCATTTATTTCCTCATCC
TCCCAAAGCAAAAAAATATTTTTCATTTACCGTTTCCTACCAAAT
TGCAAAGAATACTCCAGTCTGACTCCATTCTAGTCACACTTTATT
TCCCACAATAGATTTCATTTCCCCTACTCTGCACTAAACTAAAAT
TCTCAATCTTCCCAAATTTTTTCAACCTTTTGTTTATTTCATTAT
CCAG

In some embodiments, the intronic domain that promotes trans-splicing primarily in the heart comprises or consists of sequences from the gene MON2. In some embodiments, the sequences from gene MON2 comprise or consist of:

(SEQ ID NO: 1024)
TAAATAATTTTTCTTTTCATTGAATGCAAAGCTTTTTTATGTGTA
CTTGTCTCGAAGCACTCTATTTGTATTACAATTATGCATGTACAT
GACTCATCTCCCTTTACATTGTTAAGATTTTTGAGGGGCTTTTCA
GACCATGTTTTGTTCTATTTTGAATCCTCTACAACATTGCTTTTC
AAAGTATGGTTTGTTGGACCAGCCATTGATTGGCTTCAACAGTTT
TGTTACTGGTCTGGGACAAGATAGTGAGCTGGCATCAGAATATAA
ATCAGATGTGTCACTAAACACACTGCTTGGTTCAGCTGACTTTTT
TTTATACAAGAAGACATTGGTAAAGAAGAAAACTGTGTGTTGGTT
TACATTCTGGCACAAGCTTCTTATTTTGTAGCAGATTGGCACCTT
AAGGAGCATTACTTTTAATATCCAAGAAAAAAGTAGATTCCTAGT
AAATTACTAATGAATGAGTTGTAGGATATTTGTTCCTGTCAACTA
TTAT

In some embodiments, the intronic domain that promotes trans-splicing primarily in the heart comprises or consists of sequences from the gene BTG1. In some embodiments, the sequences from gene BTG1 comprise or consist of:

(SEQ ID NO: 1025)
CTTCTGACTGTTATTTATGAGCCTTTTCATGGTATTTATCATTTT
AGTATTGAAGATGCAGTGTTCTTTTGGGAATTACGTATGCTTAGA
AGAGAGTACTGATGCTCAAAGGTGAAATGATTTGAATCTAGAGTT
GGATAAAAAGGGAAAGGATGGGGAAAATGAAAACTGGGAAGCTAA
ACTGAGAAGGAATTGCAAGGTTTGGTGATAAAAATTCATTTGCTA
TGGTCATAGTTGTGTATCGAGAGGTTTGCACAGCACAGTCTCTTG
GCAGAGTTTAGGAATCTCTACTCTGGGGATTCTGCAGTTCATGAA
TCGTCAGGGAGACTCTGCATTCAATTTGTTTCTGTGAACATGCAG
GTGAAAGTGGTAGTACAGGTGGAACTTCATGGGAACTGGTTTTGG
GGGAGGTGTGGATGGAAAGCACAAATGTCTCAGTATTTTCTAAAG
GGAGACTATTTTTAAAGGTGTCTTGTTACAAACCACTTGATTATA
GCCT

In some embodiments, the intronic domain that promotes trans-splicing primarily in the heart comprises or consists of sequences from the gene BTG1. In some embodiments, the sequences from gene BTG1 comprise or consist of:

(SEQ ID NO: 1026)
CACACCATTCTCGAGAGTTAGAATTCGTAGACCCGCAACACATGG
ACTACCCTTGCCAATTGTTATGGTTTTATTTTTAAACCTATTCTG
TGAAAAAGCTCCTCGGTATTTCAAACTTGAATTACAATAAAAACA
CCAGGTAGGGTTGTTTTTAAATGGGTCTCACCCCAGTGCTGACTA
CTGTTGTTTACCAACTACATTTCATCTGGTAACTCCATTGAAACT
AAGTACCTCAACCATTTTTTTTTTTTATCATAAAGCCTTTTTTCA
AATGGAAAGTTGTTTTTGAAGATCTTGACATCAGACTTTTTCAAT
CTTTGAAAATGATGGAAACAGTTAATAAAAATGTTTATTTTAGAA
TTCAGGTGATGTCTGGCTTGTGAGGAGCTGTTTATAAGTTTGTCT
TTGAAAAAGGCTTGCACACATCACTCTGAGACCAGCTGTTTCCTG
TGACTTTAACCTTACCTTCCCCAGACAATTGAGCTGAACCTGTAT
CTGC

In some embodiments, the intronic domain that promotes trans-splicing primarily in the heart comprises or consists of sequences from the gene HAL. In some embodiments, the sequences from gene HAL comprise or consist of:

(SEQ ID NO: 1027)
TTATGGTGTCGCTTTATTCTTAGGTATTTATAGCAAAGTTAAGCG
CCTCTAACAGCAGTTAATCAAGCTTCTGAAGGATTCAGTTGACAG
ATGTCAACCGTTTTAAGAACAATATGCTTGACTCAGTACACTCTT
TTTCCCATTGGCTAGCAACAAAAGAGTTGGAGGTATCAGGATTTC
CTCCCCAGCCCCGCAGGCTCACCTGGTCTTTGTCACAAGTTGTCT
CAGGGCAGCTGGAGGAGGCAAACCCTAAAGAACAAGGGCTTTTCA
AGTCCCTGTTGTGCCACGTTCGCTAATGTCCTGTTGGCTAAAGCG
AGTCACGTTCCAGGCCCAGAGTCAATTTGGGAGGGTAACACTCAA
GGGCATGGGCATGTGATTTGACGTGATCCATGGGCCTGTATCTGA
CAATCTACCACATGCATAAAATCCCATCACAGGCTGGGTGCAGTG
GCTCATGCCTGTAATCCCAGCACTTTGGGAGGCCAAGGTGAGTGG
ATCA

In some embodiments, the intronic domain that promotes trans-splicing primarily in the heart comprises or consists of sequences from the gene HAL. In some embodiments, the sequences from gene HAL comprise or consist of:

(SEQ ID NO: 1028)
AGCTACTCAGGAGGCTGAGGCACAAGAATTGCTTGAACCTGGAAG
GTGGAGGTTGCAGTGAGCTGAGATCATGCTACTGCACTCCAGCCT
GGGCGACAGAGTGAGACTCTGTCTCACAAAAAAAGAAAAAAAACA
AAAAAACAAGAAAAAAACCACATAGGTACTGTTAAGACCCTGGAG
CAACTATCCTTCACTCTATCCACACTGAATTCTCTGGGTTTTGTT
ATTTGCTTGCTTGTTTTGCTTAGGAAAAGTATGCAGCTTAAAATC
TTTATGATGACTTTAGAAAGTTTTGGACTTTGTATATTTACAGTC
TTTAATGCAACCAAAGACTAATTGACACAGGTCATGTGTCAAATG
GCTTAAAATAAAAGCAAGCAAACAGCAGCTGTGTCTGGTCATGTA
TAGAGTATGTTTTGACTTCGGATGAAGTTGATGTTTAATAGCATG
TGGAAACAATGTCGTCAATATTTCAACTTTGTTTTGTCCTTGCTT
TTCA

In some embodiments, the intronic domain that promotes trans-splicing primarily in the heart comprises or consists of sequences from the gene NR1H4. In some embodiments, the sequences from gene NR1H4 comprise or consist of:

(SEQ ID NO: 1029)
ATTTGGGTGTCGAATGATGCCATGCTCATGATTTGTAGAGCATAA
TAACAATGTTAGCCTTAGGCCATTTTTATACATTATCTTGAGGCA
GAAGCTAGTTGTTACTTTGCTTTACCATAAAAATTATGACTTGTG
ACTAAAAAACTATTCAGCAGAACCTTCTAAAGTTATGTATGGATA
TGTCCTATGACCCAGTGATTTCACTTCTGAGAATATACTCAACAG
AAAGGATGCTCACGTCCACCCTACAGGTTATCTTTAGGGGTTTTG
ATTGGAAGGGGAATTGGGAGGCTTCTGGGAAGCATTACATGTGTT
GTATCTTGATCTCGTTATAATTACGTGTGTGTGCATAAAAATTCA
TCAGGTTGAACACTTAATTTTCACATTTTGTGTACAACATTTAAC
TTACACTTCAAAATAGTTAACGTTGCTATAATTATGCTGAATTAA
TGCTTTTCCACTTTTTAATTTTGTCATTTTATTTTAAACTTCAAA
ACAG

In some embodiments, the intronic domain that promotes trans-splicing primarily in the heart comprises or consists of sequences from the gene NR1H4. In some embodiments, the sequences from gene NR1H4 comprise or consist of:

(SEQ ID NO: 1030)
TTTCTTTGGGAACAAAATTTGATAAATATACAAACTCATTTCTTT
AAATGTTGAAAGACATCCTATTGAATCTGAGGGGGTAATTTACAG
AAAGATACTGTCTCTCGCACTTTTGTCATCTCTGGAAAAGTCCTG
GTGAATAGTCATTCATTTGTAATAGCCCTCTGATCATTTCCTTTT
CTTTGAAGTTCAAAGAGAATAGCAAACAGAAAAGTTAACAATTTT
CACATAAAATATTCCCTCAAGCAATCCTTTCCCCCATAGTTAGTC
TTGAGGCAGGATTCACAAATTCAGAGTATGCAGCTAACTGCAGTG
ATGGGACCTTCGCTTTCCTCTCTTAAAAGTGAGGAAATGATTGGC
TTCATTGTATCCAATCATCTTTCTGTTTTGTTTTGCTTTTGAAAA
CATTCCTACAGCTGGGAGCACAGCTGCTGTATACTTCCCCCAAAG
CAGGAATTAGTTCATTTCCTTCCTTCATTGTTACACTGTTGCATT
CTTC

In some embodiments, the intronic domain that promotes trans-splicing primarily in the heart comprises or consists of sequences from the gene IGF1. In some embodiments, the sequences from gene IGF1 comprise or consist of:

(SEQ ID NO: 1031)
ACCTTTTAGAGAATCTCGCTAAGAAACATGGAGAAAACGGAAAAG
AAAAATGTAATGCCCTAGAAAGCGCAAAGAAAGACAGTGGCAAAA
ATGAAAAAAAAAAATAAAAATTATAAAAGAGGCAAAAAAAGACAC
ACTATTCTCTGCCTCTAAAACACAATTAAATAAAAGAATTTAAAT
AAAAATTAAGGCTTCTATATGCATTTTTAAATTTTGTATGAATCT
GTTATGGAAGAATTGCCTATGTCAATATATGTTCAGAGTTAAATA
TTAGCCCCAAATGCTCAGCAAGACTGAATTGTGTCATAGAAGTTC
CCAGATTCCCTTTTCCCGCAATGTCATTGGAGGCTGCATTTCTTA
GTCAAGTCCAGGGTTTAGGCCAAAGGGCATCCGGTATTGCCTAAA
ACCCTGTGAGGTCTGTGAGGTAACTTTTGAGAAGAGGTCACTGCA
CTCTTCATCTTTTTTGCACTTTGGAATCAGATATAAAAGATGTAT
AAGT

In some embodiments, the intronic domain that promotes trans-splicing primarily in the heart comprises or consists of sequences from the gene IGF1. In some embodiments, the sequences from gene IGF1 comprise or consist of:

(SEQ ID NO: 1032)
TGGTCTTAAACCTCAGCTGAGGCTGGAAGAGGACCATGGTGGCTT
ATTGGAGATGTGACCCCAGGGAGCCCCTCTGAAGGATGGAAGGGG
ACTGGGCAAGACCCAACACACACAGAACACAGTAGCCACTGGCCA
GGCAGGAAGCAAGGATCTCAGAAAAGACTTTTAGGTGAATGTGGC
AGGAAAGCGTGCTTGCTGGGGCAAAGGCAGATTCATTCTTTCTCT
TCCCAGGTGACCCAGCGCCTCTTGGTTTCTAACTGGGGAGGGGGT
AGGTGTCAAGAGATGAGTCCCAAAGTTCTGGAATGGTGGGTCTTG
TGACTGAGGTCTAGACCCCTCTCCAGCATGAGTGCTGTCTCCTGC
ATCATATGGAGCCTGGGCATTCTGAGCTCATTCAAAGGGACACCA
TGGGAACCACTTGTTCTCAATGCAATTATTTTTGTGATGTTTACA
GTATCAGCCCCCATCTACCAACAAGAACACGAAGTCTCAGAGAAG
GAAA

In some embodiments, the intronic domain that promotes trans-splicing primarily in the heart comprises or consists of sequences from the gene HPD. In some embodiments, the sequences from gene HPD comprise or consist of:

(SEQ ID NO: 1033)
TACTGTGTCTGTGTTTGTGTTGCGCATAAGGCAGTAGTGCCTTAT
GGCAGCCAGGGGGAGGCCAGGGCGGCCGCCAGCGCCGCGGGACCG
AGGAGCTGCGGGTGCCAGCGCTTTGCCAGCGGGACCACGCGGAGA
GGGGAATTCTGGAACCAGGGAAGGAAGGGGACTTGCTCTGGAGGG
AAAGACGGATGACACTGCTTTTGTAGAGAGATAAACGCGATTCAG
ACAATAAAGCAAGCGATGAGCCGGGCGTGGTGGCTCACACCTGTA
ATTTCAGCGCTTTGGGAGGCAGAGGTGGGAGGATCGCTTGAGGCC
AGGAGTTCGAGCCCAGCCCGGTAACATAGCAAGACCCCGACCCCC
CTGCGTCTACACAAATAACAAATAAAAATTAGCCTGGCGTAGTGG
CGCACACCTGTGGTCCCAGCTACTCAGGAGGCTGAGGTGGGAGGA
TCGATTGATGGACAGAGGTCTAAGCTGCAGCGAGCCCTGATCCTG
ACAC

In some embodiments, the intronic domain that promotes trans-splicing primarily in the heart comprises or consists of sequences from the gene HPD. In some embodiments, the sequences from gene HPD comprise or consist of:

(SEQ ID NO: 1034)
ACAGCGCAAGGCTCCGTCTCAAAAAAGAAAAAGAAAGTGTCCTGA
TAATGTCTGCGGGGAGGATGGATTGTAAGGATACAAGATTAGAAG
CGGGCAGCAGTCTTGAATGGTGCCTTAGGAACTGTAGTAGGGGGA
TAGTAATAGGACAGTTTGGGGATATATTTGGAGGTGGCTGAACTG
CCAGAAAAAGAGGTCATTTCCAGACTTTTGGGGTGCAGGAGCCCC
TGTGGACTTTCCAAGGATCTCACCGTGCCAGCCTCCCCTAGCAGA
GCCCCCTGTCTCCCGCCTAGGAGCTGAAAATCCTGGTGGACTACG
ACGAGAAAGGCTACCTCCTGCAGATCTTCACCAAACCGGTGCAGG
ACCGGCCCACGCTCTTCCTGGAAGTCATCCAGCGCCACAACCACC
AGGTACTGCTTGTCCCCGGCAGGCCCGAGGGGACAGGCAGCTAGC
ACACTCGGTTTTCTGGGTCCTTGGCTCATCTCCTCTCTTTCCCGC
CACA

In some embodiments, the intronic domain that promotes trans-splicing primarily in the heart comprises or consists of sequences from the gene PHF11. In some embodiments, the sequences from gene PHF11 comprise or consist of:

(SEQ ID NO: 1035)
CTTCTCCAGTCGTTCCAGTCCTCCCCGGCCCTCTACTTGAGTGGG
TGGGTCTTCTGATGGGTTCCTGCCCTTGCGTGTGTGTATAGCTCC
AAACGGTGTGCCAGGCTCAGGGCAAGGTCCTGAGCATACAAAGAT
GAGTTAGGCAGACCCTGCCCTAGAGACTCTACTAGGCAACTTTAA
TGAGGAGGTCAGAACCACAGAGGTACAGATGAGAGACTCCTGACT
CCCAGTCTTCAGGGAAAGCTTTTTGAGGAGAGGATGGCTGGGCTT
ACCCTTTTTTTTTTTTTTTTTTTTTGAGAGGAGTTTCACTCTTGT
TGCCCAGGCTGGAGTGCAAAGGCGCTATCTCGGCCCACTGCAACC
TCTGCCTCCCGGGTTCAAGCGATTCTCCTGCCTCAGCCTCCCGAG
TGGATGGGATTACAGGCATGTGCCACCACGCCCAGCTAATTTTGT
ATTTTTAGTAGAGAGGGTTTCTCCATGTTTCATGGGCTTACCTTT
AAAG

In some embodiments, the intronic domain that promotes trans-splicing primarily in the heart comprises or consists of sequences from the gene PHF11. In some embodiments, the sequences from gene PHF11 comprise or consist of:

(SEQ ID NO: 1036)
TAAGTCACGGGAGATGCGCTTCCATCCTGACCTCTTCCTCAAACT
CCAGACTGGTATAACCAACTTCTTCCATTTCTGAAATGATCTCAA
ACTGAGTATGTCCCAAAGAGAATTCTTGTTTCCTCCACTGTATTC
AGGTACTCCAAACCTGCTCAACCACCAGCCCATTCCATTCGCCCA
GGTGCTCCTTGCAAAAACCTTAGTGGCTCCTCAGGGCCTCTCTTT
CCTCCGGACACCCCTTCTAATGCATCAGTCGGGCCAGCCAGTTCT
GTGTTCAGCCTGACTGTATCTGGAGCCTGACTGCTTCTCACCACT
TCCATTACTGCCACCCAAGCCTACGCCATCATGATCTTCTGTCTG
GACTATTGCTTTCCTGGCTTCTATTCTAGCCTTTAATACAGGCAG
GCCTGCAGAGCGGCCAAAATGCTTTTTGAAAAACAAAAAGCAAAT
GTGATTCTCCTACTCAAAACCCACCAATAGTCTCCCATGACTCTC
AGAA

In some embodiments, the intronic domain that promotes trans-splicing primarily in the heart comprises or consists of sequences from the gene CCNB1IP1. In some embodiments, the sequences from gene CCNB1IP1 comprise or consist of:

(SEQ ID NO: 1037)
TTAAGTATCATTTGTTACTATTATTTTCTCTACTGAGCGTGGAAA
GCTGGCTGAAAATTCCAGAGTGTAATTTCTGTGGGTCTGTTTCTC
TAAAACTTGCCCCTCACGCAGCTTGTACAGAAGTTAGATTGGCGC
TTACTCCCAGCTGGTGGAAGATTCGGAATTAGCTGCGCCTCTGCT
GCGTGAGAGAGGGCTCAGCTTTAAAGCTTCCCAGCTCCTCTGCTT
TCCCGCTGCCCCCTACTCTGCCGGATGTAAAATAGGCCTCTTAAT
CTTAACTGTGGCTGCCAGGTGACATACCTGAGGAAACTCACTCAG
CGGGGCTATTGGGATAAAGGTGTGCGAGTGTTCTTTTCTATCGAC
CTGGAGGGCCAAGGCAACTTTTTTTTTTTTCCTTCTGGGACGGAG
TCCGTCGCCGGCTGGAGTGCAATGGCGCGATCCCGGCTCACTGCA
AGCTCCGCCTCCCGGGTTCAAACGAGTCTCCTGCCTCAGCCTCCT
GAGT

In some embodiments, the intronic domain that promotes trans-splicing primarily in the heart comprises or consists of sequences from the gene CCNB1IP1. In some embodiments, the sequences from gene CCNB1IP1 comprise or consist of:

(SEQ ID NO: 1038)
AAAGACGGAGGGTCGCTCTTGTTGCCGTGGCTGGAGTGCAATGGT
GCGATCTCGGCTCACTGCAACCTTTGCCTCGTGGGTTCAAGTGAT
TCTCCTGCCTCAGCCTCCCGAGTAGCTGGGATTGCAGGCGTCCAC
CACCACGCCCAGCTAATTTTTGTATTTTAGTAGAGAGAGGGTTTC
ACCGTGTTGGCCACGCTGGTCGGGAACTCCTGACCTCAGGTGATT
TGCCCGCCTCAGCCTCCCAAAGTGTTGGGATTACAGGCGTGAGCC
ACCGCGCCCGGCCCACTATCGCTTAATTTAAAGAGCTGTAATGCA
TGTTTGTAATGCAGACTCAAGGAAAATTATAATTTCCTTAAATGT
GTATAGAGGATGTGTGAGAGTGGAAAGATGTCTATTCCAAGGTGG
ATAAAAAATAAAATGGTGGACTACCTCAACATGGAGGTGCCAGCT
GTCCAATTACAGCTGTCACTCTGTTTCTGGGAATTTCTCCCGATT
CTTA

In some embodiments, the intronic domain that promotes trans-splicing primarily in the heart comprises or consists of sequences from the gene PSMB5. In some embodiments, the sequences from gene PSMB5 comprise or consist of:

(SEQ ID NO: 1039)
AGGACTACTTGCATGACCAAAGCCAAGATGGCAGGTACGTCCTTT
TAGAATTTAAGGTTAGTGAAGCCATCTATTTCCCCGACCCCCTTC
AGTGAAAATGGTCTTTCGCATCTGGCCCTTCTTTTTGGAGGCGTG
CTTGCCAGCAGTCAAAATGGCTCCATTCCGGAATAGATTTAATAG
GAAGTGAAGCTGTGACGGCGAGGCGTTGCCCGGCCTATCTTTGCT
AGGCGTTCTCAGAATTAGTTCTTTCTGCCCACACTAGACATGGCG
CTTGCCAGCGTGTTGGAGAGACCGCTACCGGTGAACCAGCGCGGG
TTTTTCGGACTTGGGGGTCGTGCAGATCTGCTGGATCTAGGTCCA
GGGAGTCTCAGTGATGGTCTGAGCCTGGCCGCGCCAGGCTGGGGT
GTCCCAGAAGAGCCAGGAATCGAAATGCTTCATGGAACAACCACC
CTGGCCTTCAAGGTGTGGAGCCAGCCCCCTTGCCAGGCTGAGTAC
TGAA

In some embodiments, the intronic domain that promotes trans-splicing primarily in the heart comprises or consists of sequences from the gene PSMB5. In some embodiments, the sequences from gene PSMB5 comprise or consist of:

(SEQ ID NO: 1040)
TCTCAACCGTCCTCCCGTCTCTGAGATGGTAAATTACTACTGACA
CTTATTCAGGTCTGAACTGAGAATCTAGTTTGTGGATTAACTGGA
CCCTTTGCTTTTTCTTCTTCCATCTGCCTTTTAGGCAATTCATTA
CTTATTCGAATTTCCACCAGAAGGTAGGAGTCAAGAGAACGAATA
TTGGATATTAATAACCGGTTACTGTTAAAGAAGGGTTGCTGTATG
GCGACTGACAACAAGAGGTATAGCACTTTTATTTTGTTGCGTTGT
TTTACCACTAATTGCTGTGGTTTAACTAATATAGGGAAATGGCCT
CAGAATGGATCATTATTTTACCCAATTTTAGAAGGTGCCCTTAAA
CAAGATGGCAACCTCAGGCGCGTTAACAAGGTGCTGAAGCAGCAG
CGATGTAACCTGTTGCTTGGACACCAGGCTGGAGTGGGAGGACTC
ACCGCTAAGGGTTCAGAACCGATTTTCAACCAGTCACGCCCTTCT
CAAA

In some embodiments, the intronic domain that promotes trans-splicing primarily in the heart comprises or consists of sequences from the gene CTAGE5. In some embodiments, the sequences from gene CTAGE5 comprise or consist of:

(SEQ ID NO: 1041)
AAGTCCGGACGTGGTTGGCAGGGCGCACCGGCGCGTGCCCCGCAG
GCCATTTTTCTGACTGGAATGACGGCGGCGGCTGGCTTCTCGGGG
CTGCCGGGGTCTCCCGGGGTACGCCGCCGTCAGGACTTCTGCAGG
GCGCAGACAGCAGGCTCCAGCGAGGCTGCGGAAGGAAAGCCTTGG
GTGTCGGGGCTGGGCCTGGCGTCGGCCCCTGGGTCGGGGGGGTGG
GGGTTGCGCCGAGACGCCTCTAGGAGAAGTCTCGCCGCCGCCGCC
ACCGCGGCCGCCCGAGGCGGGTGCCCTTGGGACATAGCCCTGGTC
TTTGGGGTTGTGCGGCTCACACGACAGCGCGGAGTATGAGGCCGA
ATCTCATCCTCTAGTCCCAAGCCTCTCCACTACCAGGGCTGGGTG
GCTTCGCCAGGTAGAGCGCCGGCCCCTTTAAGAGCAAACGACCCG
GACACGTCTGCGAAGCTTGCGCGAAGAAGGGGAAGTTTGCGGCTG
TCCG

In some embodiments, the intronic domain that promotes trans-splicing primarily in the heart comprises or consists of sequences from the gene CTAGE5. In some embodiments, the sequences from gene CTAGE5 comprise or consist of:

(SEQ ID NO: 1042)
TGAGCCCAGGCGCGATGAGTGTTACGTGGCCCAGGGGTCGCGGGA
GCCGCCGGGGGAAGGAAGCAGTAGACCGGCTTTGGGGTCTAAGCC
GCCGTGGTCAGAGGTCCCGAAGCCAGTCCTCGTCTGCTGCCCGGC
TCCCGCCCGTGTTCGAGGCAGTAGTTAGGCTCGTAGGGCGCTTGA
GTGGTTTTTGTGTGATGGAGGAGGACCTGGGGCTCTGGGAAGGTG
AAGAAAGCACTTGCTTAGCTGGACTTTGCAAAGATGTAAATGTTT
CCCGATTCCTAAAACTAATTAGCCTTTCCGCTATGTTGTTTATTT
TGAGCAGAGACACGTGGTTCCTTCGACTGTGAGAAACTTGAGGAC
TGTTCCTTTAAATTAGTTACATTTACTGATGTAACTTCTCGTTGA
ACCTTGAAAGAACACCTGAGGTTGGGCGGGGAGTTGAAATTTTGA
TTCTGCATTTTTTTTTTTTTTGGTAAAGAATGAATGCCCTTTTGA
GATG

In some embodiments, the intronic domain that promotes trans-splicing primarily in the heart comprises or consists of sequences from the gene SERPINA10. In some embodiments, the sequences from gene SERPINA10 comprise or consist of:

(SEQ ID NO: 1043)
GTACTTTGATAATGTTCTGCTCTCCCAAGGCCACAGGGCCCTACG
ATTGTCTCTCCCTTTCCTTTCGTTAGGCCAGCATATGATTAACGC
TACGTGATTTTCTATGAATGTGTTTTCACGTTTCAAAAACAGATT
GATACACATATTGAACAGTGCCAGACGCTGTCATTTGAGGCCCTT
CCCTGGTATCCTATGTGCTTGTAGTCCTTATTATTTTCAGAGCAC
TCTACATAGCTCCCCTCTGACACTTAGAAGCATAGGGTCTTTCCA
AAAAACAGGGGGCTGGGGGATTATCTGGGGGATTTAGGATTGCAT
CATTGCTCCTTCATTTTTACTTTTTGACCAACTCTCTGCCCTTAG
ATTCCTATTATAGAAAATAGGGACACTCCACCTACTACAGTGTTA
GAGGCTAAATGAGACAATGAATGTAAAGTGCCCAGATGGGCTTGG
CACATAGCAGACACTGAGTATCTATTGTTTACTTGTTCTTCCAAA
CTGC

In some embodiments, the intronic domain that promotes trans-splicing primarily in the heart comprises or consists of sequences from the gene SERPINA10. In some embodiments, the sequences from gene SERPINA10 comprise or consist of:

(SEQ ID NO: 1044)
TCCCCCTTCCTTGCCTAACGGGCATGCAGGCACTCCAATCCCCAG
CCAAACAGGGCACTGGGCAAGGCCGGCCACCCATCTGGATGGGCA
GCCTGACGACCAGATGGTCAGGGCAGTGAATGAAGCAGATCAAGG
AAAGGTGTGTGAGGACCCCTGATTCCACCTGCTTGGACCCCCACC
TTCTGTGCTGCCTCCTGCTCCCAGAGTGGACTCTCTTGCCCTGGC
CCTCAGGGAGGAGACGGGATGAATGAAAACGGGGTCAGGACTGAG
AGCTGCCTGCCGGCCTGGCAGGGAATGGGAACTGGAGGAGGTTTT
GCTCTGTGAAATAATGTCCCCTCTTTGGGTGAGCAAATGTCACCC
ACACTTGCTCTAGGTCTCCCTGGGGCAGGGCTAACCTACTTGAGC
CACAGGAAGGAGGCAGGGTCCCTGAAGAAGCTTTTACTATCCACA
AAGACATTTTAGGAGGCATTAAAACCATCTCTATCCTCTCCTCTC
CACA

In some embodiments, the intronic domain that promotes trans-splicing primarily in the heart comprises or consists of sequences from the gene SERPINA5. In some embodiments, the sequences from gene SERPINA5 comprise or consist of:

(SEQ ID NO: 1045)
TCCCCTCATTCTTCTGAGCCCCAATTCAGTGATTGGTGCAATCAC
AGGCTTGGCTACAGTGACCCATTCATTGCAGGCATGGTGAGTCTC
TCAATCCCTCTCATTTCCACTAGAATCTAACTGTTGGGATCTATG
ACCCAGTCAGCATAGCAGGCCTGTGGGGAGCTCTCAGGTTCAAGC
ATATGCCCCCCCTAATCTACAAGAAATTAGCTGCAGAAAACCAAG
GAATAGAACCTGGAAAAAGAGAGGGTTTGCTAGAGCTGTCCCTTT
CCCTGTCTCTGGAATGCCAACAATAGGGAGGCTCTTTGGTCTTGT
CTCTCAGGAGTGCCCATGCCATTCCAGGAAAATGATGGCCCAGCT
GGTGGTGTAAGGCTTGGGGGGCAGCGAGTGGGCATCGTGGTGAAA
GCCTCGGGATCAGGGAGCTGCGTCTGCAGGCAGGCCTGCTGGCCG
GAAACCTGCCAGGAAAGGAAGGGGCTGTCCCGGGGCGGGGCCAGG
GAGG

In some embodiments, the intronic domain that promotes trans-splicing primarily in the heart comprises or consists of sequences from the gene SERPINA5. In some embodiments, the sequences from gene SERPINA5 comprise or consist of:

(SEQ ID NO: 1046)
TAAGTACCGATGCCGCAAAGGGAGGTCCCCAGGGCTTGAGGGCAT
GTGAGGCGAGGAGAGGATGGACTCTAGAGTTTTGGGGTTTGGGGT
CTGCAAAGCTCTGAAGGAGTCTCATCTCTGCAGTTTCAGGTATCC
AAGGCAGCAGAGGTGAGTGGGTCCCCCGAGCTCTGTGACCTTATG
CTCCACACTAACTCTGGCAGAGCCTCCGTTTCCTCATAGGTAAGA
TGGAAATAATTACACCCTCTGGATGGTGTGACTGAAGATTAAATA
CAGCGGGTGCTCTCACTCAGCACATCTGGCCATGTCTGCAGACAC
ATTTGGTTGCCACAACTGGAAGGGGGGTGGGGGTTAGTGACATCT
AGAGGCCAGCGATGCTGCTGATGATCCCACAATGCCCAGGACAAG
ATCACAAAGCATCATCCTGTTCAAAAGGTCAACAGGATCAAGGTT
GAGAGACCCTGAAATAAGGCCATGGGGACAAAATGTCGGCTGGAT
AGGA

In some embodiments, the intronic domain that promotes trans-splicing primarily in the heart comprises or consists of sequences from the gene PPP2R5C. In some embodiments, the sequences from gene PPP2R5C comprise or consist of:

(SEQ ID NO: 1047)
AGCGTGGTGGCGGGTGCCTGTAATCCCAGCTACTAGGGAGGTTGA
GGCAGGAGAATCACTTAAACCTGGAGACGGAGGTTGCAGTGAGCC
GAGATCACGCCACTGCACACCAGCCTGGGCAGCAGAGTGAGACTC
TGTCTCAAAAAAAAAAAAAAAAGTGAGCAATATCTGTGGTTAATC
TCAAATAGGGTGGCTTTCCGTGTCATTTCCCTTTAGTTGTTTTTA
AATGCAGATGTTGCTTTAATTAAATAATATTTGAAATAAGTGGGG
TCCCAACTATAACCTTATATCTTATTGTTTTTAAATGTTGATATT
TTGGTGTTTTCAGTAGAATTTGAGCTGTTAACAGTGATTAGGTTT
TTTCAGTTTGTCATCGATGTATGTGACCTCGTTATAATACACATG
GATTTTTGTCTGCTGGTTGTGTTACTCATGAGCAATGTGTTCTAA
TTATGGTATTTTAACTTATAACTTATGAAAAAGTTATGTTTCACT
CTAG

In some embodiments, the intronic domain that promotes trans-splicing primarily in the heart comprises or consists of sequences from the gene PPP2R5C. In some embodiments, the sequences from gene PPP2R5C comprise or consist of:

(SEQ ID NO: 1048)
TGGGTGGTGAGGGTGTCTGTATATGTGTGTTTACATTTGTAGACA
GTTTCTGTACATGTATAATTAGGAATGGGTTGTCGGTTTTGGTTT
TTTTCCCAGCTTTATGTAATTGGTCAGACTTCTCAAAGGGGGGTA
GCACTTGATAATCGCTCTCTTGGAACTCTGAAGCCTTTGCATGGA
GCAGTGTTCACCCTGGGCAGCCTGTGGCCCCTGGGGTCACAGAGC
TGCTTTCCAGGGGGTTTGCAAGGCCGTCACTGCCTCATAAGATTA
CCAAGATCTAATTTACCTCTCACTGTGTGGACGTTTGCCCTGGTG
GTGCAAAGGCAATGGTGAGGAATATGGCGCCTTTCTTTCATCGAG
CAGGACAGCGGTGTTCAGCTAGCAACACACCAGTCTGACCCAAGA
ATGTCCTCGATAAAGCATGAAGTTACTGGTTTTTTAAGTCTCAAC
TCTTTTGTATACCTTTTTAATTTTGTTTTTGTTTGGGTTTGTTTT
TAGT

In some embodiments, the intronic domain that promotes trans-splicing primarily in the heart comprises or consists of sequences from the gene SERF2. In some embodiments, the sequences from gene SERF2 comprise or consist of:

(SEQ ID NO: 1049)
AGGATCCGGCGTCCGGAGCCCGGGATCACCTGCCTAGCGGGCTTC
ACTTGGAGAGGTGGGGAGGCCAGAGCTTGCGGAAGCCGCTCCCTC
TCCCCTGCACCTTCTCCAGGGTCGCCGGCTGAGGCCCAAGGTCCC
TAGGGGCAGTGACTAGAGAAGAAGGGGGCCGGCCGTACCTGTTGC
CGCTGACACTCTTGCAGCGCCCGCAGGGCAGCGTCGTTGAGCCTG
AGCAGTAAGAGGCTAGTCCGGGCAGCTTGCGTGAAGCAGAGCCGG
AGCTGTCCTCTCAGAGGCTCCTGGAGCTCCTCCATGGCGACGAGT
TCGAGTGCAAGCAGCACGGGGGCCACAGGCGAGGGCCACCACCGC
CACCTCCTCTGTTCAGGGTTTGGTTGGCACACCCGGTAACCCACG
TCTGCCCCACCCTCCGCCAGGAACCCGCCCCCGGCCCCACCTCCC
GCCAGCGCCACCCCAAGCTTTCCCAGCCGCGTGGGCGGCGCTTCG
CTCT

In some embodiments, the intronic domain that promotes trans-splicing primarily in the heart comprises or consists of sequences from the gene SERF2. In some embodiments, the sequences from gene SERF2 comprise or consist of:

(SEQ ID NO: 1050)
TAATTGGGGCCCTGCGGTTGTGGTGGCAGCAGTGAAAGTTGGCTG
TCACCGACTGGAGCGCCCCCAGTTGGTGGGCAGGGATACGGGCGC
GGGTAGTGGCAGGAAAGTTGCAGGGCAGAGAGCCAGAACCCAGTC
CTAGGTCTCACCTCGTGGCCTAAACACGCGTCAGAGTGGTGAGAC
CGCTCCCTGAGGAGGGCCAGGAGCTGCCATGAAGTCGCCAGGCAT
TCCAGAGGGCCAGATCCTGGTGCACTGAGACCCCAGCACCAACCT
CTGGCCAGCCCTTGGTGTCTCCACTTGTTCCTGTTTGATTATGTT
TCCCAAGAGCCCCGATGTTGTCCTGTTTCCTAAACAGGATCCCTC
TGTTAAATCCCTGGACCTGCACAATCCCCGGGAACCTTAACGTCC
TCTACTACAAATAAAATTACCTATACATTTGTCAAAACTCATAAA
AAAAAAATACATTTTTTTTTTTTTTTTTGAGACGAAGTCTTGCTC
TGTC

In some embodiments, the intronic domain that promotes trans-splicing primarily in the heart comprises or consists of sequences from the gene AQP9. In some embodiments, the sequences from gene AQP9 comprise or consist of:

(SEQ ID NO: 1051)
GCCCGCTGCTGTGGCAAGAATGGCCTGGGGTCAGTGCTAGCTGTG
GCAGTGGCTGCAATACCCCTGGCTCTAAGGGCATCTTCAGTATAC
TGCAACACCCTCAAATTCTGGAGTGGTATTAGGGGTTACTGAGAG
CCCTCTGGGAATTATAAATTATTCCTAGAGCAAGACCAAGCCCCA
CTTTGGCTTACAGTTGACCTCGAAAACTCCAAGAATTCCCCCAAA
AGAGAACTCATTCCCAGGACCCTATCGAAGAATAATTTGTCCAAG
TTTGCAGTGGGCCATTCAGAGAGGCCAAAGGCCTTCTTCCTTTGA
CCCTCCATCCTGGGATTCACCTGGGCATCTCTAAAACTATCATTC
CTCGGAAGTAGAGAGAGACAAATGACATGGCTATGCCAAGCTCAA
TCTGAGTGACTGGTTGTTTAGCACAAGGCTTGGCACAGGCCTCCT
TCAACTCTTCCCAGGGAACACTAATGTGCCAGAGCTTTCTCTTTC
ATAG

In some embodiments, the intronic domain that promotes trans-splicing primarily in the heart comprises or consists of sequences from the gene AQP9. In some embodiments, the sequences from gene AQP9 comprise or consist of:

(SEQ ID NO: 1052)
TAAGTGTAGATTCAACAAAGACTTAACTTTGGTGAAAAGATATGC
TCTCATAAGGGGAATGCATTGGAGAATTAGAGACCAATCAGAAAG
GAGAGATTATATTTCCAAAGGCAGAGGTTGCTGGGCATAACCCAA
GCTTTCTTGATAATCGTTTTACCTTTACAAGGAGCTGATACCACC
AGAAAAGTTTTCTCCTTCCTCTTTAATACGTACACTTGTTTAACA
TTAGTTGAGATCCAGAAAAGAGTGAATGACTAAACATTTCAATCC
CTGTAGACACAAATGGACTTCACTTCACTATTGACTCTTTTGAAC
AAGTGGTCTAAGTGTGGAGGCCACAAGAGAAGATATTTCTATTTC
AAATCAAAGACTGTGAAGGGTGGTAGTTGAGCCTAGGGGATCATC
AATCCTCTCTGTCTCACCATGTGCATAGGTTTATTTATTTAAACT
CTCAAGGTGGCTAGAAGTATCTCTGCTTATTGACCTTTAAACAAG
CTTC

In some embodiments, the intronic domain that promotes trans-splicing primarily in the heart comprises or consists of sequences from the gene ANPEP. In some embodiments, the sequences from gene ANPEP comprise or consist of:

(SEQ ID NO: 1053)
GTAATCCAGCCCTGCCTTATGGGCAGCAGCCCCCACCCCATACTC
CTAGCTCCTGGCCCATGTTCTGAGGGCTGTGATGGGCCATTCCAT
TCAACAAATGGACTTGACTTCTTTGGTTCCAATCTCCACTTTGCT
GCTGCTTCGCTGTGTGACTCTGGGCAAGTTACCATCCCGCTCTGG
TCTCTAGTTCCTCTTCTATAAAATGAGGCTAAGAGCAATACTCCA
TTAGTTATGTGAGGATTAAAGGAGAGAATCCTAGGCCAGGCATGG
TGGCTCATGCCTGTAATCCCAGCACTTTGGGAGGCCGAGGCGACC
TGGGGTCAGGAGTTCGAGACCAGCCTGACAAACATGGAGAAACCC
CATTTCTACTAAAAATACAAAATTAGCCAGGTGTGGTGGTGCATG
CCTGTAATCCCAGCTACTCTGGAGGCTGGGGCAGGAGAATTGCTT
GAACCTGGGAGGCAGAGGTTGCAGTGAGCCAAGATCGCACCATTG
CACT

In some embodiments, the intronic domain that promotes trans-splicing primarily in the heart comprises or consists of sequences from the gene ANPEP. In some embodiments, the sequences from gene ANPEP comprise or consist of:

(SEQ ID NO: 1054)
CGAGAATCTGCAGGAGATGCCCTGGATGGCAGGGACTGGAGGGTC
CCACTGATGGCCACGCCCTCTCGCTCCACCCACAGGCTGTGAACA
ACCGGTCCATCCAACTCCCCACCACCGTGCGGGACATCATGAACC
GCTGGACCCTGCAGATGGGCTTCCCGGTCATCACGGTGGATACCA
GCACGGGGACCCTTTCCCAGGAGCACTTCCTCCTTGACCCCGATT
CCAATGTTACCCGCCCCTCAGAATTCAAGTGAGCAGATGGGGTCA
CTGAGGCATGGTCAGGTGGGTGGGGTAGAGGGCAGTTACTAAGGG
ACCATAGTAGGACTGGCCTCCTGTGGTCCCAGCCCCAGCCACTGT
AGACAGACCCTCCCTCCAGAGCCAGGAGTTCAGGGGCACTCGGTG
CCAGAGCTGGGCAGAAGCTGGGAGCCTGGGCAGCTGCTCCTCACT
GGCAGTGACCACACCGCCATCTCTCACCACCTCTGGATCTTTAAT
TGCA

In some embodiments, the intronic domain that promotes trans-splicing primarily in the heart comprises or consists of sequences from the gene PCSK6. In some embodiments, the sequences from gene PCSK6 comprise or consist of:

(SEQ ID NO: 1055)
ATGAAAATGAAAATGACAGATCTGAGGAAGAGGGAGAGCTCTCTT
GAAACCCCTTTCCCAGATTTGCCCTCAGTTTTAGGAATGAGGTAC
GCTTGGGCATTCCTGTTGCCTGCATGGTTCGGCATTCTGGCAGCC
AGGTTCGCCCCAGAATAGATAAGTGTGTTTGATTTTCGAACATTT
GACTTTATTACCTATTTCCAGAAACACTTCGTGTTCAAACTACGT
CTCTACAGGCATTTCTACTTGGTCTTTTTCTCCCCTGACATTTCA
CAGAGTCCTAACTGTGCTGCATGTATAACTTGTTCTTTCTTTTTG
TTACTTGCATAGCAAGCATGTTTCTATGTCATTAAATATCCTTTG
TAAGCAGCATTTTATAGATTTTTATAATACTTCACCAACAGAAGC
ACCATAATTTATTTAAGCATCTCTCTATTGTTGGACATGTAGATT
GCTTTCCATTTTTTCCTATTATGAGCGGACAGCTTAGTACAAAAA
GCCT

In some embodiments, the intronic domain that promotes trans-splicing primarily in the heart comprises or consists of sequences from the gene PCSK6. In some embodiments, the sequences from gene PCSK6 comprise or consist of:

(SEQ ID NO: 1056)
CACTTCCTTGCCTCGGCTCCCCCAGCCTCCCCCACGGTGAAGCCT
GGGCGTTGGAATGGCTGCTGACGTTCCCACCGGGGAGGAGCAGCG
GAGGCGCAGGGGCCGGTCCGACCCAGCGGGACCCCGGGGGGGCGG
GGGTTGTGGCTTTTCCCTCCACTTCCAGGTGAGGCTTGGTATCAG
GCCTCCCCAGCATCTGGCTGCCTGGAGGGGGCAGGTAACACAACC
CAGAAGGAAGGTTTACCCAAGGAAAGACAGGACATAAAGTGTATG
CCCTTCCTGTCTGAGGGCACATTGGTCTCACAAGGCCTGTTTGGA
AGACTTCCCCCAGCTAAGCAACCACCCCGTTTCTTTGTGGAGCTG
CAGCCAGCTTGAAACACAGCTCCTTCCATTTGTCAACCCCGGGCG
CTTCCTCCTGCTGCCCTCCACTCCTGTTGCCCTGGGATTGCACCT
CCCAATAAAGCTTCAGCCCATGGGCTTCACTTCAGTCCCTTGTTT
TCCA

In some embodiments, the intronic domain that promotes trans-splicing primarily in the heart comprises or consists of sequences from the gene ACSM5. In some embodiments, the sequences from gene ACSM5 comprise or consist of:

(SEQ ID NO: 1057)
GTTAACTATGGAAAGAAAAGAAGAAAGACAGGCAAGAAAGAGGGAAGAA
AATAGGAAGAAAATGAAGAAGGAAGGGAGGGAGGAAATAAAGGAAAGAG
AAAGGAAAAGAAGGAGGAAAGGAAGGGAAAAAGATGAATGGAAGGAAGG
AAATAGGGAACAAAGAAGAAGGGAGGAGAGAGGAAAATGGAGAATGAAA
TAACGGGGAAAAGAATGAAAAAAAGGAGGAAAGAAAATGAAGGAGGGAA
GGAAGAAAACAGGAAAGGGGAAAGAAAGGAAAGAAAGAGGAAAGGAAGG
AGTGAGCAAGGAAGAAAAAAGAAAAGAAAGAAGGGAGGAAGAATGGAGA
GAAATGGAGAGAGGAGAAGGGAAAGAAAGAAGAAAGTGAGGGAGGAGAA
AAAAGAGGATGAGAGAAAGAGTTATCAGGCAGGGTGAGAGAAAGGCATG
GCCCAGGAAAGAAGCTAAGTGGAAAGGCTCTTCTTTATCTGTACTGCGT
TCTCCCCAG

In some embodiments, the intronic domain that promotes trans-splicing primarily in the heart comprises or consists of sequences from the gene ACSM5. In some embodiments, the sequences from gene ACSM5 comprise or consist of:

(SEQ ID NO: 1058)
TAGCAGCCTCCCCCAACTTCTGGCAAGGCCTGGCATGGAGAGGAGAAAG
ACACACAGGCCTGGTTGGCACGGGCTGCTGGGCTGCTGGGAGAGGCCCA
GGGTGTAGACACTCACGTATGCACCTCTGTGATGATTTCCTTACCAGAT
TGTGGATGATGAGGGCAACGTCCTGCCTCCTGGAGAAGAGGGGAATGTT
GCCGTCCGTATCAGACCCACTCGGCCCTTCTGTTTCTTCAATTGCTATT
TGGTAAGAGACGGGGAACAGCCGTTCTCATGACAGTGACTGTGTGCTAA
GCATTGTGCACCACACTTTGTAAATATCTATTTGTTTAATCCTTACAAT
GACTGCATGAGGTAGGTTGCTACTATTATTAATAAAGTTCTGTTATTGA
TAAGGAAGACTATTATTGATAAGGAAGCTACTACAAAGGGGAAGTGACT
TATCCAGCATCATGCAAATAGTACAGGCAGGACATGGGATTTAAATGTG
GGTGTGTCT

In some embodiments, the intronic domain that promotes trans-splicing primarily in the heart comprises or consists of sequences from the gene ACSM3. In some embodiments, the sequences from gene ACSM3 comprise or consist of:

(SEQ ID NO: 1059)
GCTGTGGATCAGAGGACCCTGTGCCAGATCATACAACTCCTTGCAGTTC
ATGTAAGGACTCGGATTTTACCTGGAGTGGAAAAAGAAGCACTGAAAGA
TTTGAGCAGGGGAGTAACCTGATAGCGTTTATGTTTAGTCCTGCCACTT
CGACAGATAAACGCACCAATGGGCTTGATGAGATTTAGGCCAACCCATA
ACCGCCCCTCAACTTCTTTCCTTTCAATTTCAAAACTCCTCTATGGCTT
CCTCCATCTGTTCTTCCTTCTGAGAAGTGCTCTCTCTGCCCCTTTACAG
AACTAACCACTTCGGCAACTCCTTGGACACTTTCCTTCTTGTTAATAAT
TTGCTTTCTCCGCCCCTCAAAAGCTTGCTGTTTCTGTAAATCATTACCT
GTAAGAGGAACCGCTGGGAGTCCTGTAAACTTTAGCCCAGAGCTTGGCT
CCTCCTCCAGAATGTCTCCACCAATCAAGGAAAGTGTTTTGGGCCAGTC
TTGCTCCTC

In some embodiments, the intronic domain that promotes trans-splicing primarily in the heart comprises or consists of sequences from the gene ACSM3. In some embodiments, the sequences from gene ACSM3 comprise or consist of:

(SEQ ID NO: 1060)
TGAGTACCTCATCTCCCCTCCTGTGTAAGATTTCCTGAACTGGCACATC
TGTTTTTTGAGCAAAGATAACAAACAGATGAACAAAACCAACAATCAAA
AATGCTGTCATTAAAGTCTTGGGCAGCCAAAGTTTCTCTCAGAATTTCT
CAGTTGTGTGATACTATCTATTAAGTGATGAGGAGTATGCACACACAAA
AGGCTATAAATGTAGCAGCTGAGTTTTCATGTTGAGCCTTTTGGTGCTA
TTTGATTTTTTGAAAAACTATGTACATGTATTAAGTTGATAAATTTTTT
TTTTAATTTTAATTGAACCAGATGCGGTGGCTCAAGCCTGTAATCCCAC
CACTTTAGGAGGCTATGGTGGGCAGATGCAGATCACTTGAGGCCAGGAG
TTCGAGACCAGCTTGGCCAACATGGTGAAACCCTATCTCTACTGAAAAT
AAAAAATTAGCCAGGTGTGGTGGCAGGTGCCTGTAACCCCAGCTACTTG
GGAGCCTGA

In some embodiments, the intronic domain that promotes trans-splicing primarily in the heart comprises or consists of sequences from the gene TMED6. In some embodiments, the sequences from gene TMED6 comprise or consist of:

(SEQ ID NO: 1061)
GTCTGTTTTCATTACCATGGCTATTATAATAATCATAGGGTCCTATGAA
ATTACAGAAATAATGAGGAAGTATACAGAGATGTTTTAACAGTAGGAAA
AAAATTCAGCCTTTAACACCAAAAACCAGTGTTCCTGGAGGCTGCTGAC
CTCCAAGGATGCCAAGTGGCAGTCTACTGACGATTTGGGGGCAACACTC
CAGGTGAGCATTCTAAGTCTAGGGTTCAGCCGCTCAATTTTATCTCATC
ATCTCCAGTTAATCAGATTCTGGGAAAGTGACCAAAAAACAGAGACCCA
TTTATAATACAGAATTCCAATTAACAAATGGATTACAGGGAAGCATGTC
TTTCCAGTCATCAGAATATTTGAGTCAGTTACTAAAAGCAGATTCAGAA
CAAAAGAAAATGCATGATATCCATGTGAGATATCCACACTCCACAGCAA
AGCACTCCTCCAGCCAGGTCAAGCCTGTCCCACTTTGGAGAGCAGCAGT
GTCTTAACT

In some embodiments, the intronic domain that promotes trans-splicing primarily in the heart comprises or consists of sequences from the gene TMED6. In some embodiments, the sequences from gene TMED6 comprise or consist of:

(SEQ ID NO: 1062)
TATAGGCGCACGCCACCATGCCAGGCTAATTTTTGTATTTTTTAAGTAG
AGACAGGGTTTCACCATGTTAGCCAGGCTGGTCTCGAACTCCTGACCTT
GTAATCTGCCCACCTCGGCCTCCCAAAGTGCTGGGATTACAGGCGTGAG
CCACCGCGCCCGGCCCAGAATCATACATTTTAGTGGATCCACACTAGCT
GTGTGTTCCGAGCAAGGCATGTCTTCTTCACACGGGTGCTGTTGGCATT
CTGGGCAGTTCTTTGTCCTGCTGTACTGTTTCTTACATTGCAGACGTTC
AGCAATGCTGTGCTGCCTCCCCTTAACACCATCACTGTGATACCTCAGA
ATCCTCCAATCAACTCCAAACCCCAGCAGGGGGTTGGGCAGTCCCACCC
CAGACAGAGACCGCTGTGCTGATGACCGTGGGACGTCAATCTTACCACT
CAGCTGGCTTCCTCGTGATGTAGCAGGGGTTCTGCTACCTATTTAATGT
GTCTTGGCA

In some embodiments, the intronic domain that promotes trans-splicing primarily in the heart comprises or consists of sequences from the gene HPR. In some embodiments, the sequences from gene HPR comprise or consist of:

(SEQ ID NO: 1063)
AGGGGGCTGGGCGTGATGGCTCACACCTGTAATCTCAACACTTTGGGAG
GCTAAGGCGGGTGGATCACTTGAGGGTGGGAGTTTGAGATCAGCCAGAG
CAACATGGAGAAACCTCGTCTCTACTAAAAATACAAAAAATTAGCCAGG
CATGGTGATATATGCCTGTAATCCCAGCTACTCGGGAGGCTGAGGCAGG
AGAATTGCTTGAACCTCGGAGGTAGAAGTTGTGGTGAGCTGAGATCATG
CCATTGTACTCCAGCCTGGGCAACAGGAGTGAAACTCTGTCTCAAAAAA
AACAAAAAACAAAAAAGAACACCACGGGAAAATCAAGTGTGAAGCAAGA
GCTCAACTCTTAACAGGGGCAATGTTTGGAGTTTTGTTACTGGAAAAGA
TAGAGACCTTACCAGGTCCAAAGTTTGTAGACACAGGAATTACGAAATG
GAGAAGGGGGAGAAGTGAGCTAGTGGCAGCATAAAAAGACCAGCAGATG
CCCCACAGC

In some embodiments, the intronic domain that promotes trans-splicing primarily in the heart comprises or consists of sequences from the gene HPR. In some embodiments, the sequences from gene HPR comprise or consist of:

(SEQ ID NO: 1064)
TGGGTCCACAGCTTTCCCTCCTGCCTTTCCTCTGGTTCTTTATTTCAGT
CTTTTTTGCATACATTGGTACAGATGCAGAAATAGAACAAAGAAACAGG
GCAAATGGGCTAAATTATAGTGAACCAAAGGGCTTAGTGTGTTAAATCT
TCTCCTTTTCTGCATCCATAGAAGACAGTGCTGCTGTCTTTCCCAGGAG
ATAAGATTTACTCTCAGGAGTGTCTTTTTCCTTGAGGTTACGTTTTTGT
CTTTGTAGGGTATGTCATCAGCTCCCGTGGTAGGCTTCCTGGCATTCGG
AATATATTTACTAGCAGATATTTTCCTCTTTAAAAATGTATAATAAGAA
AGACTAATAGTAACACATTTGAATGACACAATTAATTGACTAGTACTTG
GGATACACACTAGTACCTGGGATACATCTAATTAAGACACTTAGATCTT
AAAAAATAAAAAGACTTTTTGAAATGTTGAAATAATAAGACTAGAAACT
TTTTTTTTT

In some embodiments, the intronic domain that promotes trans-splicing primarily in the heart comprises or consists of sequences from the gene SERPINF2. In some embodiments, the sequences from gene SERPINF2 comprise or consist of:

(SEQ ID NO: 1065)
TGTCCCCCAGGCTGGAGTGCAGTGGCTTGATCTTGGCTCATGGCAACCT
CTGCCTCCCGGGTTCAAGCAATTCTCCTGCCTCAGCCTCCCGAGTTGCT
GCGATTACAAGCGCCCGTCACCACAGAAGCCCAGCTCATTTTTGTATTT
TTAGTAGAGATGAGGTTTTGCCATGTTGGTCAGGCTGGTCTTGAACTCC
TGACCTCAGGTGATCTGCCTGCCTCGGCCTCCCAAAGTGCTAGGATTAC
AGGCGTGAGCCCCCATGCCCGGCCACACTGCTAGAAACTTTAAAAGCAT
CTCATTCGGTTACGTGTCCGCCTGCAGTGGGACAGGTAGTATGACTCCA
CCCCACTGTGAGGAAGTGGAGGAGCAGTGGGGTCAGTCACACGCCTGGC
AGGATGGCCAGGAGCCCCATTGTCTGCCTTAGGAGCACCTGCTGGCCCC
ACCCCCACTTAGCTTCGGGGCTTTCTGTCCTCATGCTCTTCCCTTCCCT
TTTCTGTAG

In some embodiments, the intronic domain that promotes trans-splicing primarily in the heart comprises or consists of sequences from the gene SERPINF2. In some embodiments, the sequences from gene SERPINF2 comprise or consist of:

(SEQ ID NO: 1066)
TAAGGAGGAGGGTGCGGGCGAGCCCCCGAGGTCAGGCTGGGCAGGGCGG
GTAAGGAGGAAGCGTCTGGCTGGCAGTGGAGCTGAGTCAGAGCTGTCCT
GTGTCCTGCTCTTTTCCAGGGCTTCGGGAGCGGGGAGAGGGTTGAATAT
GAGCCCCCAGACCCTCTGTCCTAGAGGAAGTTGGGGTGGCCCGGGGAAT
GGGGCTCTGCTTTGCTGCCCAAGACCTCGCATTGCAAGGAGAGTCTGGG
TAGAGAGCCTGGGATGGCATGTGAGCCTCGCTGGCTTCCCTGGAAGGGG
AAGGAGGAGAAGCTGGGGTGTCACACTGTTAAAGAAATGCTGGGTGGAG
TGGCTCATGCCTGTAATCCCAGCACGTTGGGAAGCTGAAGTGGGTGGAT
TGCTTGAGCCCGGGAGTCCGACACCAGCCTGTGCAACACATTGAAACTC
CGTCCCTACAAAAAATACAAAAATTAGCCAGGTGTGGTGCATGCTACTG
GGGAGGTTG

In some embodiments, the intronic domain that promotes trans-splicing primarily in the heart comprises or consists of sequences from the gene SLC13A5. In some embodiments, the sequences from gene SLC13A5 comprise or consist of:

(SEQ ID NO: 1067)
CTGTGCTGCTGACTTGTCTCTGGTTTTCTGCAGTGTGGGGAGCCCAGGG
AGGTGGATGAATGAACAGTTAGTTACGCCCTGCCCACCTGCTGGGTGCC
AGGCCTTCCTGTCCCTGTTGAATCCACTAGTTATCTCGTTCGATCTTTG
CAGCAACCCTGTGAGATAGGAAGGTGTTATTATCTTGCTTTGTCTTTCA
AAAAAAAAGCGAGGCTCAGGGAGGCCAAGGGAAGTGTCCAAAGTCACAC
ATCAAGTTACTGGCAGTTACAGTTCCAACCAAGAGCTTCCAACTCCATA
CCCCCTGCTCCTTCTGCTAGCCATGAAGGGCTTTGGCCTTATAGGGCTT
GTAGGGAAAGGTGAGTGGCCAAGAGCAAGTCCATGCCAAGGGAAGATCT
CCAAACATGAGTCCCTGTCTGTTGCCTCCCCTGAGATAGGCACAGGACA
AGTGATCAATGAGACAGGGTGGTCCTTGCCCTAAGAAGCAAAGTGTTTG
GTTGGGGAG

In some embodiments, the intronic domain that promotes trans-splicing primarily in the heart comprises or consists of sequences from the gene SLC13A5. In some embodiments, the sequences from gene SLC13A5 comprise or consist of:

(SEQ ID NO: 1068)
CCATGGACTGCAAACTTGACTCCAATCCCCAGTAAAATATCTACAACAG
GGTAGTGAAGCGATGGTTAGTGACCATGAGGGAAGCTTGCAGAGCAGGC
ATCAGAAAGAGCCTGAGGAGGTCCACAGGGAAGCTGGCACGTCCTTGTA
GGATAGTTAAGGCACTGGGGTGAGCAATGAACCTGGACTCACGGAACAC
TGGGCTCTGTGACCGTTTCCCTGAATGGCCTAAGCTGTTGCCTCCTGTC
ACTTCTCTGAGGTCATTTTCCAAATGCGCACGGGCATAGAGAACCCATC
CACTCTGCCTACTTCCCAGGGATGCCTTGAGCACTGAGGATACCTGGGG
GACATGAAGTCGCACTGTCCTGGGGGTCGGGACACCCCAGCCAGGGACA
GAGCATGGCACAGGGACATCGAGGCCCAGTGAGCCGACCCTTTGTCCTC
CTCTCTGAGAGCACTAGTCCCCAGCAGGCCTCAGGGTGCTGACTCTGTC
TCTTTTCCA

In some embodiments, the intronic domain that promotes trans-splicing primarily in the heart comprises or consists of sequences from the gene ASGR2. In some embodiments, the sequences from gene ASGR2 comprise or consist of:

(SEQ ID NO: 1069)
GTGAGGCCACGCTTCCCACAGGCAGGAGGAAGCCCTTGGAAGGCCTGCT
CTGGGAGGCTGGATCAGGCTTATTTTATCCATTTTTTTTTTTTTTTTGA
GATGGAGTCTCGCTCTGTCGCCCAAGCTGGAGTGCAGTGGCACGATCTC
GGCTCACTGCAAGCTCCGCCTTCTGGGTTCACGCCATTCTCCTGCCTCG
GCCTCCCAAAGTGCTGGGATTACAGGCACGTGCCACCATGCCCGGCTAA
TTTTTTTGTATTTTTAGTAGAGACGGGGTTTCACCGTCTTAGCCAGGCT
GGTCTGGAACTCCTGACCTCATGATCCGCCCACCTCCGCCTCCCAAAGT
GGTGGGATTACAGGCGTGAACCACCGCGTCTGGCTGAACTTCAAAGTAT
TTTAATGCCACAGTAGTTAAGCCATATGATACAGGGGCCAGAATATATG
TATTGTTTGCATTACATCAGGCTCTGTTAGTACTTTACAGCATACTGTG
AGTACGAAC

In some embodiments, the intronic domain that promotes trans-splicing primarily in the heart comprises or consists of sequences from the gene ASGR2. In some embodiments, the sequences from gene ASGR2 comprise or consist of:

(SEQ ID NO: 1070)
TGTGTAGTGTGGTGTATATGTGTGTGTTGTGTGTGTATGCATGTGTGGT
GGTGTGTGTGAGATGAGTCTGTGTGTGTGCGGTATGGTATATGTGTGTG
TACGCATGTGATGTGTGTGGTGTGTCTCTTGGTGTGTTTGTGTGTGTGT
GATGGATCTGTGTGTGGTGTGGTCTATGTGTGGTGTACATGTCTGTGGT
GTGTGTGTGGTATGGTGTGTGGTGTGGTCTATGCATGGTGTACGTGTCT
GTGGGTGTATATGCTTCCCCAGCCACAGGCTGGAGCCTAGCAGGTGGGG
ACCACACCTGCCGGCAGCACTTTCAGCCTGTCTGTCCTAGATCACGATG
CCCTGCTCTTCCATCTGAAGCACTTCCCCGTGGACCTGCGCTTCGTGGC
CTGCCAGATGGAGCTCCTCCACAGCAACGGTAGGGGTGGGGCCGTCTCC
AGGCCCAGTGTGCCTGCCCCTCCGGCTGGATCTCATCTCCCTGCCCCCT
GTCCCCTCA

In some embodiments, the intronic domain that promotes trans-splicing primarily in the heart comprises or consists of sequences from the gene TMEM220. In some embodiments, the sequences from gene TMEM220 comprise or consist of:

(SEQ ID NO: 1071)
GTCAGGCGGCCGCGGTGGGAGGGGGACCCGCCCGGCTCCCAGCTCCGCC
CCCTGGCCTAGCGGGCCGTGTAACTGACTAGCGGGCAGAGTCGCAGGGG
CGCACAGTTAGTGGGGAGGACGGGACTTGGGCTTTCTGCTTGGGGAGCC
CCCACTTTCTCCCCCGTCTAAGAGACGCGGATGTGTTCATGGAAGCGAG
TCCCATACCAGGGCTCACACCCTGGAAGGGGCTGGTTCCGCGCGTGGAA
CTTCCCAGGGGACCTCAGAAGAAGCGGCCCTGGCTCCTGCGTTAACGTC
CGGCCCGCGGACGACACTGAACATATCTCAGGGCCTAGGAGACGAAGCC
GACCAAAACCCTTTATAGCATCATTAAACTTTTTAGTCGGCTTGGGGAC
CCCCTTCATAATCATTTCCCTCATTTCTTGAGACTGGTGGGGGTGGAGG
GCCCTTAAAATACCCGTTCTGGGTTGGCGAGTCCTCTCCAGAGCTCCCG
TGAAGGCTT

In some embodiments, the intronic domain that promotes trans-splicing primarily in the heart comprises or consists of sequences from the gene TMEM220. In some embodiments, the sequences from gene TMEM220 comprise or consist of:

(SEQ ID NO: 1072)
TGAGCCGTATAAGATGTTAATATAACTGTCGTCACTCTAGAAATT
ACCATAATGCTTACAGGGATTTGAAAGTTCCTGAGCTTTGGAAGG
AAATGCATAAATACTAAACAGAACACACACACACACACACACACA
CACACACACACACACACACACATCTCTTTTTCTTTCTCTTTCTTA
TTTTTGTAAAAACAATTTTTATCCTTTTTTTTTTGAGGAGTCTCG
CTTTGTTGCCCAGGCTGGAGTGCAGTGGCACAATCTGGGCTCACT
GCAACCTCCGCCGCCCGGGTTCAAGCAATTCTCCTGCCTCAGCCT
CCTGAGTAGCTGGGATTACAGGCTCCCGCCACCACGCCCGACTAA
TTTTTTCGTATTTTTAGTAGAGATACGGGGTTTCACCATGTTGGC
CAGGCTGCTTTCGAACTCCTGACCTCAGGTGATCCGCCCACCTCG
GCCTCCCACAGTGCTGGGATTACAGGCGTGAGCCACTGCGCCCAG
CCCT

In some embodiments, the intronic domain that promotes trans-splicing primarily in the heart comprises or consists of sequences from the gene NCOR1. In some embodiments, the sequences from gene NCOR1 comprise or consist of:

(SEQ ID NO: 1073)
TCTGAATTTTCAGAGTGAGAATTGTTGTCCTGTCCTTGAAGCAAA
TCACCTTCAAGTATTACAGTGTAATATTCACATTCTCTGGTAGAT
GAGAGACACAACTTTGTGTAGTGGATATTTCCAATCAGACGCTGG
CTAAGCCTTCACCTTGGCATTTGCCAGCTGTGTGGCCACAGGCAA
GTCACTTAACCTTTCATAGCTTTAGTTTTCTCATCTGTAAAATGA
AAATAATAGTACACGTCTACCTTGCTGTGGTTGTAAAGTTAAGGA
AGGAGCATTCGGTATTGTTATATAACAGGTGGCTCTGCATTATTT
TATGTATAATGTAGCACGTTATTACATATTAATTAGTGTATTAGT
ACTGTATATACACAGTAGATACTATGGAGGTACCTGTTTTCATTG
TTACATCACTGTTTTTATTTCAAGTGATGGATTTGAGTTGGTCCG
TTGCTGTTTTAGTTGGTGTGTGATACACTTTTACTTTCTTTAAAA
AAAG

In some embodiments, the intronic domain that promotes trans-splicing primarily in the heart comprises or consists of sequences from the gene NCOR1. In some embodiments, the sequences from gene NCOR1 comprise or consist of:

(SEQ ID NO: 1074)
GGCATGCCTCACTGCACCCAGCTAAATTTTTGTATTTTTGGTACA
AAGATACAAAATACCAGCCAGGCTGGTCTTGAACTCCCGACCTCA
GGTGATCTGCCTGCCTCGGCCTCCCAAAGTGATGGGATTACAGGC
ATGAGCCACTGCAATGCCTTAAGCATTATTCTTGAAGATAACTTT
GATTAAAAAATGATAAAATCAATTCAACCAATAACATGACAGAAA
TAAGCATTACATTACTTGTTAAGAGATGGCAATTATTACATTGGT
TGTTTGCATATTTCGATTTATTTTCTTCCTATAGGTATCCTGCAC
CCCATGTAACATACCAATTGGAACCCCAGTGAGCGGCTATGCTCT
CTACCAGCGACACATTAAAGCAATGCATGAGTCAGCACTCCTGGA
GGAGCAGCGGCAGAGACAAGAACAGATAGATTTGGAATGTAGAAG
TTCTACAAGTCCATGTGGCACATCCAAGAGTCCAAACAGAGAGTG
GGAA

In some embodiments, the intronic domain that promotes trans-splicing primarily in the heart comprises or consists of sequences from the gene UBB. In some embodiments, the sequences from gene UBB comprise or consist of:

(SEQ ID NO: 1075)
TAAACTGCCTCTCGGGAGGTTGGGCGCGGCGAACTACTTTCAGCG
GCGCACGGAGACGGCGTCTACGTGAGGGGTGATAAGTGACGCAAC
ACTCGTTGCATAAATTTGCGCTCCGCCAGCCCGGAGCATTTAGGG
GCGGTTGGCTTTGTTGGGTGAGCTTGTTTGTGTCCCTGTGGGTGG
ACGTGGTTGGTGATTGGCAGGATCCTGGTATCCGCTAACAGGTAC
TGGCCCGCAGCCGTAACGACCTTGGGGGGGTGTGAGAGGGGGGAA
TGGGTGAGGTCAAGGTGGAGGCTTCTTGGGGTTGGGTGGGCCGCT
GAGGGGAGGGCGTGGGGGAGGGGAGGGCGAGGTGACGCGGCGCTG
GGCCTTTCCGGGACAGTGGGCCTTGTTGACCTGAGGGGGGCGAGG
GCGGTTGGCGCGCGCGCGCGTTGACGGAAACTAACGGACGCCTAA
CCGATCGGCGATTCTGTCGAGTTTACTTCGCGGGGAAGGCGGAAA
AGAG

In some embodiments, the intronic domain that promotes trans-splicing primarily in the heart comprises or consists of sequences from the gene UBB. In some embodiments, the sequences from gene UBB comprise or consist of:

(SEQ ID NO: 1076)
GTTTGTGTGGTTTCTGGAAGCCTTTACTTTGGAATCCCAGTGTGA
GAAAGGTGCCCCTTCTTGTGTTTCAATGGGATTTTTATTTCGCGA
GTCTTGTGGGTTTGGTTTTGTTTTCAGTTTGCCTAACACCGTGCT
TAGGTTTGAGGCAGATTGGAGTTCGGTCGGGGGAGTTTGAATATC
CGGAACAGTTAGTGGGGAAAGCTGTGGACGCTTGGTAAGAGAGCG
CTCTGGATTTTCCGCTGTTGACGTTGAAACCTTGAATGACGAATT
TCGTATTAAGTGACTTAGCCTTGTAAAATTGAGGGGAGGCTTGCG
GAATATTAACGTATTTAAGGCATTTTGAAGGAATAGTTGCTAATT
TTGAAGAATATTAGGTGTAAAAGCAAGAAATACAATGATCCTGAG
GTGACACGCTTATGTTTTACTTTTAAACTAGGTCAAAATGCAGAT
CTTCGTGAAAACCCTTACCGGCAAGACCATCACCCTTGAGGTGGA
GCCC

In some embodiments, the intronic domain that promotes trans-splicing primarily in the heart comprises or consists of sequences from the gene LRRC75A-AS1. In some embodiments, the sequences from gene LRRC75A-AS1 comprise or consist of:

(SEQ ID NO: 1077)
CCTGAGGTCAGGAGTTCAAGACCAGCCTGGCTGGCCGACATGGTG
AAACCCCATCTCTACCAAAAATACAAAAATTAGCCGGGTGTGGTG
GCGTGTACTTGTAATCCCAGCTACTTTGGGGGCTGAGCCAGGAGA
ATCGCTTGAACCCGGGAGGCGGAGGTTGTAGTGAGTCGAAATCGT
GCCACTGCACTCCAGCCTGGGCAACAGCGAGACTCCATCTCAAAA
AAAAAAAAAAAATTACAACCTACTGAGAAGCTAATTAAGATTTGT
AGTCCTCAATAATTTACAAAAACTTAGAAAAATTACCTCTAATTA
GGTCTTAAGGGGCTTTTGTTAGGATGGACTTAAATTTGTAAAGTC
ATTAATTGACTGTCATCAGAAATGCCTGTCCTGCTCTGCATCTGC
AGTCTCTAAGGTGTATGCGCCTTCTCCATTCCCCCAGGTTTTGAG
AACAGAGTGCTGTTTTAGAGCTGGCAGCAGCATCTCAGCCCAAGA
GAAG

In some embodiments, the intronic domain that promotes trans-splicing primarily in the heart comprises or consists of sequences from the gene LRRC75A-AS1. In some embodiments, the sequences from gene LRRC75A-AS1 comprise or consist of:

(SEQ ID NO: 1078)
AAAAAAAGATATCAAATGATGAAATCACCCAAAATAGCTGGAATT
ACCGGCAGATTGTGTAGTGGTGAACCTATGGTTTTCTGAAGATAT
CTCTTACCCTGTGATTGACTGTCAGTTGGTGCAGAAAGTGAGATT
GAAATGTAGTTTCTTTGCAGGTTATATTCCCAGAGGATGTCAGTC
CCAAGGACCAGTAGCTGCCATCAGTTTGGATTCTGAAAACTAACT
GGCATCAACACTGGGTGTAGAAACATGCTTGCCTTATGTATCAGA
GGACATGCTCAGCAGATCCAAGAGATATATTTGGCAACTTTTTCT
AGAAAAGGCACATTGGGTATCATTCATTACATTCTTGAGTTTTTT
TGGGTTTTTTTTTTTTTTTTTGAGACAGTCTTGCTGTATTGCCCA
GGCTGGAGTGTGGTGGCACAATCACAGCTCATTGCATCCTCAATC
ACCCAGGCCTAAGCAATCCTCCCACCTTGTAGCTGGGACTACAGC
TCAC

In some embodiments, the intronic domain that promotes trans-splicing primarily in the heart comprises or consists of sequences from the gene SKAP1. In some embodiments, the sequences from gene SKAP1 comprise or consist of:

(SEQ ID NO: 1079)
GTAAGGACAGAAAGTGTGGGGCTTAAATAGAAAGTCAATTTTTTC
ATTCTTGATTTAGTCACCATAAACAGAATTATTCCATGTCATACA
CTTCCCATGGCTCCCTCACAATATAAATTCCCATTTATTCATAAT
TGTTAGGAAAAGTTTTAAATGAAAATCCAGATTCAGGAACCATTT
TCACATTTAAAAATACAAGGCTAGGCCGGGCGTGGTGGCTCACGC
CTGCAATCCTAGCACTTTGGGAGGCCGAGGCTGGTGAACTGCCTC
AGCTCAGGAGTTCAAGACCAGCCTGGGCAACATGCTGAAACCCAG
TCTCTACTAAACTACAGAAAATTAACCAGGTGTGGTAGCACATGC
CTGTAGTCCCAGCTACTTGGGAGGCTGAGGCATGAGAATTGCTTG
AACCCGGGAGGCGGAGCTTGCAGTGAGCTAAGATCACGCCACTGC
ACTCCAGCCTGGCGATGGAGCAAGACTCCATCTCAAAAATAAATA
AATA

In some embodiments, the intronic domain that promotes trans-splicing primarily in the heart comprises or consists of sequences from the gene SKAP1. In some embodiments, the sequences from gene SKAP1 comprise or consist of:

(SEQ ID NO: 1080)
CAAAGTTCCTATGGACAAAGAAGGCCGAAAAGTGAGCTGTGATCC
AGAAGCCTGCTGGCTGACTAAGCCTTTCTTGTAAATAGGCAGAAG
TCACAGATTTACTCTGATCCTTCAAATGCATAATGAAGAGAAAAA
CTAGATAGTTAGTTGCCCTGGATCAGGAAAGTCTGGCGGGAAACC
TCTGCACTGCTTACTTACAAACCAAGTGAAAAATTACCTGCCTGA
TTTTCTTTTATTAATAAATAAATATATTTATCATATTGTTGAACG
GCATCCTACTAGGAGTGATCTGTAAATATTTAATGTTAGTTAATG
ATCTCCTTCTCCCATAGTCCTTTGCTCATGCTGAGTGTTTGGCTG
TATCTCATCTCAACTCCCCTTAATCTGAGGTTTCCTAGGGGCAAT
ATCAGAACCCTGGTTTCCATTTGCAGCAAAAACATGGGTACCTCG
GAAAAAATAGAGTCCTTTTAACTTTGATTTCTCCTCTCTTTGATC
AGTA

In some embodiments, the intronic domain that promotes trans-splicing primarily in the heart comprises or consists of sequences from the gene TOM1L1. In some embodiments, the sequences from gene TOM comprise or consist of:

(SEQ ID NO: 1081)
ATTCACCAACAGTTCAGTGATACTTGAATTCTGGTCTTCTTTCCC
AATCAGGCTGCTCCTATTATTCAGAGTCCTCAAATACTTGCTCTA
TGCATTCCGTCCACCTTTAAGTGACATTCAGTGGAGAGACAGAGT
GCAGTACGTTTGCTCCGCTTTATCCTGGACCGGAGACCTCACAGC
TTTCTTCTTAACCTGTGTTCTATACTACTTCTCATCCTTTAAGAA
ACGATGGGGTGGTAGCTACACTGATGGAAAGACTAAAGGCTCTGG
TCTTAGTGTTCCGTGCACACTCCACAGTCTCAGGGCTCCCACCAC
CCATGGCATCATAACCTGATGTGCACTAAAACCAATTGTTTTATA
AACAGTTATTACAAAATGACACTTTATGCAGTCTGAAGGTACCTT
TTAGGTTTTGTTTTTAGACTAGGAAAATTATCTTACTTTTTGCAT
TTGTGTTCTCACTGTAGGGTAATCATTCTGTGTTATCACACTATC
CTAG

In some embodiments, the intronic domain that promotes trans-splicing primarily in the heart comprises or consists of sequences from the gene TOM1L1. In some embodiments, the sequences from gene TOM comprise or consist of:

(SEQ ID NO: 1082)
TAAGTAACAAAACTCTTTTCTAGCAGTGCATCTAGTCAGCAGATT
ATTGTAGCCAGAAAAGAACTCATGGTCATTAATTCCCATGTTCTG
CTCAGTATTGTGTTGATGCGCAGCCCCTGTTAGCAAGATGGTAAG
GATCAAAATTTTTTTGGAGTTTGTATTTGCTATCTCCACTTCCTT
TCCTTGTATTCTCTTTGACTTCAGACATGCTCTTCTACCACTCTA
TGGACATAACTCTTGTCAAGGTCACTGGTGACTTCTGTATTGTGA
AATTTAGAACTGCAGTTCCATAGTCCCTCATTTTATCAGCAACAA
ATGACAAAGTTGATCACTTCCTCCTTCTTGCACACATGGCTTTCA
GACACCAGTCACTTTGGTTCTTCTCCTGCCTCACGTTCATTTCTT
CTCCATCTCTTTTGGTGGCTCTTCCTCCTCTACTGACCTTTTCTT
GTTGGAGACCCCAGGGATCAGGCTTCAGACTTTTCTATCTACATT
CATT

In some embodiments, the intronic domain that promotes trans-splicing primarily in the heart comprises or consists of sequences from the gene ABCA6. In some embodiments, the sequences from gene ABCA6 comprise or consist of:

(SEQ ID NO: 1083)
GTAAGTTGATACATTTATTACCACTTTCTCTGCTTTAGATCTTAG
CTATATGTAGCTAATATTAATGCCCTAGTAGGTTACTGCAATCAC
TACTTTCATGTCCATTGTGTGTGTGCATGTGTGTGTCCTTGTGTG
TACTTGAAATAGTTTCTATGGAGTGAAAAAGTTATGTAATCCAAA
TAAAATTTGACTTGCCTTTTGCATAAAGGTCTGAACTAATGAAAT
GATGTATCAAAAAGAAAAAACAATGCTGACTCTAAAATATCAAAG
TCTTTTCTGTATAATTATTGAGGTTATTTTTGAGGGGTGGACCCA
ATTTTTTTTCCCTTAAGGGTGCTCATCACATGGTAGCTGATCACA
ATATCTCTTCTCCCTCTAATATTCTGTCTCATACTTACTTCTCGT
TTCACTCCGCTCTGCCTTCTCTGAACAAGAACCTCTAATTTCCAT
CTTCCAGGTTGTTTTCTGCAGAAAGTATGTAGATGCTCCTTCTTA
CATT

In some embodiments, the intronic domain that promotes trans-splicing primarily in the heart comprises or consists of sequences from the gene ABCA6. In some embodiments, the sequences from gene ABCA6 comprise or consist of:

(SEQ ID NO: 1084)
TCTGCCTTCTAGGTTCAAGCGATTTTTCTGCCTCAGCCTCCCGAG
TAGCTGGGACTACAGGCATGCACCACCATGCCTGGCTAATTTTTT
TGCATTTTTAGTAGAGACGGGTTTCACCATGTTGGTCGGGCTGGT
CTGGAACTCTTGACCTCAAATGATCTGCCCACCTCAGCCTCCCAA
AGTGCTGAAATTACAGGCATGAGCCACCGTGCCTGGCCTTGGCAT
CCTTTCTATTGGTTGGTGCCTATACCAGTGTTCAGCATCTATCAT
CTCTGTATGCATATATGGTAAAGGTAACGAAGATGAATGGCAATT
ATAAAGAAGGAGAATTGCGATTGAAAAAGGATATGCAATGAGCTT
CTGCTGTAGGATTAACAGTATAGTTATTAATCTAGAAGGTACAAG
GGTAGTCATTACATTGTTCTTTAGATGCTTTTGTATATATTAAAT
ATTTTGTAAATAAGTTAACATAATTTATATAACTTTTTGTCTTTA
TTTA

In some embodiments, the intronic domain that promotes trans-splicing primarily in the heart comprises or consists of sequences from the gene AFMID. In some embodiments, the sequences from gene AFMID comprise or consist of:

(SEQ ID NO: 1085)
GCTTGGGAGGCTGAGGCAGGAGGATCGCTTGAGCCCAGGAGGTCG
AGGCTACAGTGATCCATGATCGCACCATTGCACTCCAGCCTGGGC
GACAGAGCGAGGCCCTATCGCTTAGAAAACAAAAAGTTACATGAA
GTTCCCCCAAGGTCCCGTAGCTAGCATGTGATGTGGCAGGGAGAG
AGCCTGTCCGCTTCACTCTCTCACATGGGACCAGAAAGGGGTGAT
GCTGTGCGTGGACAGGACATCTGGCAAGTGGTGTGTCCAGGACAC
AGGAAGCAGCGTAGTGGCCAAGCTGCCTCCAGCTGTCACATCTGT
GTGTCTCTGCAGTAAGGATGAGTCTGCCTTCATGGTCCACCCGCT
GACGGCACAGGGAGTGGCCGTGGTAATAGTGGCTTACGGCATCGC
CCCCAAAGGTAATAGGAGTGGTTGCTGCAGGTCCGAGGGCCGGTG
GGCTTTAGGAGGAAGTGCATCCCTGACCCAGCTCATGCTCTCTGT
GCAG

In some embodiments, the intronic domain that promotes trans-splicing primarily in the heart comprises or consists of sequences from the gene AFMID. In some embodiments, the sequences from gene AFMID comprise or consist of:

(SEQ ID NO: 1086)
TGGGTGTTGCCAGTAGATTTTCTTCCTGTTGGACCTCAGTGGGTG
GGAGGACAGTGCAATCAGTACCTAGGATACAGCCAAGCTGCCCCT
CTGGCCTGTGGAGCAGAAAGCAAATTGGGACTCTTCGAAGGGGGC
CTGATGTTGTGGGGAAACTGCGTGCAAATCCAGCTCTCTGCTCTG
ACCCCTCCCAGGGGAATTTACCTGTGTGGACACTCAGCCGGGGCC
CACCTGGCTGCCATGATGCTCCTGGCCGACTGGACCAAGCATGGG
GTCACGCCCAACCTCAGAGGTTTCCATGGGAGCTACAGCCTGGCT
GGGCAACCTTCATCTCCCCATGAGCCTTGGGGTTTGGGCCAACTG
CTTGAAATCCTCCCGGCCATGAGTGGCTTGACCGCAGGGCTTCGA
GCCCTCTGAGCAAGGCCTTCTCTGCCTCCTCTGTGCCCCTTCCCC
TGGTCCTGCCCCTCTGGCGGTGGGGGTGGGCTGGCCCTGCCTTGC
TCCG

In some embodiments, the intronic domain that promotes trans-splicing primarily in the heart comprises or consists of sequences from the gene TTC39C. In some embodiments, the sequences from gene TTC39C comprise or consist of:

(SEQ ID NO: 1087)
AAGAACTGTTGATCCCAAAAGGGAAAAATTAGACATAGGGTGTGACGTG
TCTAGATACCTCCACCTGCGCTCATGCACACATTCCTGCGCCATAAAGG
ATTGATTTTTTTTTTTTAACCTTGGTGTTTTGTGTTTGAGCTGTGTGCT
AACAGTCAGTGTCAATGTCTGCCTTCTGAGCTGTGGGCGATTCCCATTG
TTGTGAGGTTTCCTCCAACTGGACGTTAAGTTACTGAGAACAGGAATCA
GGGATGCGATTGGTGCGTGTCTTCTGTGGCTACCTGGTCAGCATTCATA
AACGGTGATAATGGATTGAATGCTGCAGTTTCAACCTTCCTGACAACAG
AGGAGAGTGTTACTTTGGGGCATGAACAGTTATTGATCCAGCTGGGCCT
TGGTTAATGAGTCATTTGGCTTACAGTGTTCCCGAGTCACAGGCTGTCC
TGAAGATCACCTGCTGACTCATTGGTGCCCTTGTTTCCCCGAGGCCTCC
TGCAGTGAC

In some embodiments, the intronic domain that promotes trans-splicing primarily in the heart comprises or consists of sequences from the gene TTC39C. In some embodiments, the sequences from gene TTC39C comprise or consist of:

(SEQ ID NO: 1088)
TATGTGACATTTAGCATTTCTCAATGCCTAAGATTAAACTGATGCTTTG
AATCCTGATCATGCTTTTGAGCTGCTCCTCTTGGGTACTTGTGGCATCG
TGTTGGCAGACCCTGAGTGACATCTTCAGCGGCGGGAAAACGAAATGTA
TTTCACTGGGAGTAAGTAGAGCGGACATTTGAGAGTTTGCTGGGTCCCA
GACAGTGTTATGTGAATGAAAGTGTTGGAAATGTAATCTGGATCCTCAG
GGAATTTCTGAAGTGTCGGGAGAGGAGATTTGATGAGCAGAAGCAATTG
GAGAACAGCATTCTAGGGTATAGAAGCAAACTATCAACTGTATGGTGAA
GAGCTCACTGAGAACTGGGCTAATGGAGCGGCTTCGTGCATTCACTCAG
TAGCGATTGAGAGACTTCTGGGCCAGGGCTGGGCGCACACGGGGATGAA
ATATGGTCCCTTCCCTTTCCACACCTGTGTGGTAACAAGGGGCTAGATT
AGCAAACAA

In some embodiments, the intronic domain that promotes trans-splicing primarily in the heart comprises or consists of sequences from the gene MOCOS. In some embodiments, the sequences from gene MOCOS comprise or consist of:

(SEQ ID NO: 1089)
GTTCAGTACTGAAGACCTAAACATTTGTCACAAGGGCAGTACCATCCAC
TATTTACACACATTTAAAATTATTAATGTGAGGTCCAGGTTTGCCAGTT
ATAACAAACTTGTTTTCTTAAAAGTGAGTTAGTGGTGCCAGGCGTGGTG
GCTCATGCCTGTAATCCCAGCACTTTGGGAGGCTGAGGCAGGTGGATTA
CTTGAGCCCAGGAGCTCAAGACAAGCCTGGGCAACATGGTGAAACTCCA
TCTCTGCAAAAAATACAAAAATTAGTCAGGCATGGTGGTGAATGCCTGT
CGTCCCAGCTACTTGGGAGGCTGAGGTGAGAGGATCACCTGAGCCCAGG
AGGTTGAGGCTGCAGTGAGCTGTGATCATACCACTGCACTCCAGCCTGG
GTGACAGAGTGAGTGAGACCGTGTCTCAAAAAAAAAAAAAAAAAAAAGA
GTGAATTGAGAATTTACATAAAGCTCATGATTCTCTTGTGTTTCATGAT
TGATTTGAG

In some embodiments, the intronic domain that promotes trans-splicing primarily in the heart comprises or consists of sequences from the gene MOCOS. In some embodiments, the sequences from gene MOCOS comprise or consist of:

(SEQ ID NO: 1090)
TGGGTTTTCTTTCCATCCTGCTGACTTTATTACAACTCATCCTAGTTCC
AGACGAGAGCAATGTAGTGTGACAAAGTCCATGCACTGTTGAAGAGGCC
ACCAGGCCCTCAGCCACATGCCTTTCCACCTTCATTCACCCTCGTTTTT
CAGTCCTCTTTACTCAGCCAAAAGTGGTAACTTTCAAATGGGAAGAAAG
AGGCCAGTTCTTCTGATGGCAGGTGGGAAACTGTTGGGTCTTTGCCATA
GTAGAATAAGCTGTTCACTTCACTTCCTGGTCCACTAAAATGTCCAGAG
TTGCACTGAGCACTCAGGCATGGTGGTAGGTGGTTATCAGACGGGGTGT
CACAGATGGACAACAGGAACCAAATGTTACTCCCAGAGCAGCACTGTGG
GGAATGGATTCAATCGTTGGGTGTTTGGAAGACGGTGCTGGTTTTTCCT
TGAAAGCACATCTGCATCCCCATTTTTATTTATTTTTTTGAGGTGGGGT
CTCGCTCTT

In some embodiments, the intronic domain that promotes trans-splicing primarily in the heart comprises or consists of sequences from the gene PET100. In some embodiments, the sequences from gene PET100 comprise or consist of:

(SEQ ID NO: 1091)
CTAATGTCTGCCTCCCATAAGCCGACCCTGCCCTGATGTGGGGCTCTGG
AGGCCTTTGGCTCGTGTCCCATTTGTGACTTTTCCTTACAGAGGGAGCT
GTGGCCACCTGAGAAGGTAAGTGATCTCTTCTTCCTGCCAGAGGGATGG
AGGAGGCTGGATTCTTGTATCCGGGATGGGTTTTAGATGAAAATTTTAG
GCCAGGCGCAGTGGCTCATGCTTGTAATCCCAGGGCATGGGAGGCTGAG
GTGGGTAGATCACTTGAGGTCAGGAGTTCAAAACCAGCCTGGCCAACAT
GGCGAAACCCCATGTCTACAGAAATACAAAACTTAGCCAGGCATGGTGG
CAGGCGCCTATAATTCCAGCTGCTTGGGAGGCTGAGGGAGAAGAATCAC
TTGAACCCAGGAGGTGGAGGTTGCAGTGAGCCCAAGATCACGCCACTGC
ACTCCAGCCTGGGCAATAGAGTGAGACTTTGTCTCAAAAAGAAACATTT
TACTCTCAG

In some embodiments, the intronic domain that promotes trans-splicing primarily in the heart comprises or consists of sequences from the gene PET100. In some embodiments, the sequences from gene PET100 comprise or consist of:

(SEQ ID NO: 1092)
TAATTGGCAGGGGGCTTCCAGTGAAGACACTGAACCCTGAAGCCGTGGT
CTGGGGCAGGGTGGTGGGAGAGGTGGGCGGGGGGGGGTGGTCCCGGCTC
TGAGGTGTGTGCCCCTCCCCCCTTCCTGTCCCACCCGCTTCTCCACCCC
TAGCTTCAAGAGATAGAGGAATTCAAAGAGAGGTTACGGAAGCGGCGGG
AGGAGAAGCTCCTTCGCGACGCCCAGCAGAACTCCTGAGGCCTCCAAGT
GGGAGTCCTAGCCCCTCCCCTGATGAAATATACATATACTCAGTTCCTT
GTTATTCATTTAAGTGTTTTATTCTTTTATCAGTTTTTGGGGGCCGAGT
GAGACCCAGGATGCCTCAGGCCCTCAGGGGGCTTGTGTCGGGGGCTGGG
GGCTGCTGTTCCGACGGAAGCCGAGAGCGGTCGGGTCCTGAGGGGTGAG
CAGGGGTTGGGGACTGAGGGTCCCAGCTCGTTTCTGTGCTCCGTCCTGT
GGATGAAGA

In some embodiments, the intronic domain that promotes trans-splicing primarily in the heart comprises or consists of sequences from the gene PRKACA. In some embodiments, the sequences from gene PRKACA comprise or consist of:

(SEQ ID NO: 1093)
GTGAGGCTGAGTCCTGTTACTATAGCAACTTCCTAGGCACACTCTGCCC
CTTAAGTCAGGGAGGGGCTGCAGCGGGGTCTGGGGCACAGGCGAAGAGA
AGAAGGATGTGAACCCCTGGCTGGCCCTGGAAACGCCTCCTGTACAGCC
CCGGGCTGTGCGGCCTGGGGAAGAGAGAGGGGATCGGTGGCCCAGTTCA
GACAAGGGAGGATGGTCCACCCGGCAGGGGGCCAGCTCAGAGAGGGGTG
GTGGAGTGGTCCATCCCAGTGTTGGGGTTCAGCTCAGACTGTAGGATGA
GCCATCCCAGTGTTGGGGTCCAGCTCAGAATGGGGGTCGTTCCAGCCAG
TGTTGGGGTCCAGCTCAGAGTTGTGTCATTTAGGTAAGCCCAGAGCAGG
GAATCCAGGCTCCACTGGAGGAATCAGCCAGCCTCGAGTAGGGGGCCCA
GCCCAGGCTGGGCATTCCTGCCCCAGAATTCCCAGGACAGCCAGATTTC
AAAAGGGCA

In some embodiments, the intronic domain that promotes trans-splicing primarily in the heart comprises or consists of sequences from the gene PRKACA. In some embodiments, the sequences from gene PRKACA comprise or consist of:

(SEQ ID NO: 1094)
CAGCGGCCCCAGGGGGCAGGCGCCCAGGTCAGACAGCCAAGTGGGGCTC
AGGTCGTGGCTGGCACTGGCTGAAGATACCCCTGGGAGCTGGCCCACAG
GGGAAGGGGCAGAGCTAGTAAAAGGGAGCCCCCGATCCCCAGGACACAA
GCAGATCCTGGCAAAGAGAGAAAAGCACCCCCCAAGAAGACCCTAGATG
GGAAGAACTAGTGCCGTGTAACATCCTATGGATGCCTAAGACGGCCAAA
CACCTTCTGTGCCTTGGCAGTTGTATGGGTGCCCAAGAGGTCTTATCAA
GGCAGCTCTCCCCTTAGCGCAACCGCGTCTACAGAAACTCGGAGGGCCC
TATCTGTCCCTCCTCCACACCCAGTGGCCTCTGGGTTGGGTTTCTCTTC
CTGCTCCCACCCCACGGCTCCCTAGCTCCCCCTGCAGGCAGGGTTCTGG
GGACAGACAGCCGAACAGACACGGCAGGTCTCATGAGCCTTCCCAGCCA
CCGTAGTGC

In some embodiments, the intronic domain that promotes trans-splicing primarily in the heart comprises or consists of sequences from the gene SLC27A5. In some embodiments, the sequences from gene SLC27A5 comprise or consist of:

(SEQ ID NO: 1095)
GTCCGAGGCAGGGTTTCTCGGGGCGGGGCTGGCGCGGGGCCACGGACGC
TTTCAGATCCTAGAGGGAGTCAGGCTGAAGAGGCGGGACTTTCCTGTCG
TAAGAGGGCGGAACTTGGAGTCAGGACTTCTCACGCGAAGGCGGGGCTT
CTCGATTTTCGAGGGGGCGGGGCTGACTGGGCCTCCTGATCCCCAAAAG
GGGCGGGGCGGGGGCAAGAGGCGGGGCCTCTCCTTCCCGGGGGCGTGGT
CACAAGCGCCGGCTCCCTCCTTGCAGATGGAAGGGCGAGAACGTGTCCA
CGCACGAGGTGGAGGGCGTGTTGTCGCAGGTGGACTTCTTGCAACAGGT
TAACGTGTATGGCGTGTGCGTGCCAGGTGCGGAGGGTTCTCATTTTAAA
CTTCCAACAGCTTTGTGGGTGGAGGGGTCGTTATTCTGATTTTACAGGT
GGGGAAGTGCTAGGGCCCAGAGAGGTTTAGAGACTTGCTGGCTCTCACA
GCCAGTGAG

In some embodiments, the intronic domain that promotes trans-splicing primarily in the heart comprises or consists of sequences from the gene SLC27A5. In some embodiments, the sequences from gene SLC27A5 comprise or consist of:

(SEQ ID NO: 1096)
TTACCACCATCTGGGGCCTCCGCCTCAGCTTCTCGGCTCTGTCCAGCTC
CCCTGCCTTGATCTCTTGCTCTGATTTACTGGGTCTCTGGGTGTCCATC
TGATTTGTTCTCTTGAGCTTTCTGTGGAAGACGGCATTCCAGCCATTGG
TCCCTTGTCCTGTACCTTCTGTTAGTGTCTTCTCCCAGGGCCTTGCAAG
GTCCTGGTCATATCTATTCTCACTGCCATTTGTCTGTCCGTCTTCCATC
CCTTGTTATCCTTTTCCTGTTTCTCATGGGCTCCCCATACCGCCTTCTC
TATACTTCTCTCTCATTTTCTGTGTCGCTTGTCGCAGTCTCTCAAGGTC
TCTGAATGTCTCTCTCATCTCTGGATCTCCCTGTCTGTGTCTTTGGGTC
TGTCCTTGGCAGGGTGCTGGGTGATGAAGAGTCCACCGTTGTTGAAAAC
CTCCAGCAGGGGGCTTGGCTGGTGGGCTCCAGGGGTTCTTGTTCCTGCT
CCTACCCCA

In some embodiments, the intronic domain that promotes trans-splicing primarily in the heart comprises or consists of sequences from the gene ACSS2. In some embodiments, the sequences from gene ACSS2 comprise or consist of:

(SEQ ID NO: 1097)
CACTTGATGAGGTCTGCACCTTCTCAGTGATGTCCACTCCCACAGTGTA
GTTGGCAGCTCCTCACTGGAGTTTGAGTCCTCTGGTAGGGTCCACGTCT
CCTTAGTAAAGTCTATATCCTCTGTATCTCTTAGGGGAAATCCATGTCT
CCTCACTAGGGTAAGTGCCCTTCAAAGCCATTCATGTCCTATTAGCAGG
GCCCAGGGCCCTTCAGTCAGATCTGAGAACCCCCAGCAACATCTGTGCT
CCCTTAGTGGAGTCCATGTACTGTCAGTGAAGTGTGTGTCCCTTCGGTG
CAGTGAATATCTCCTTTATGAAAGTATCTGACCCCTTGGTGGGGTCTAT
GTCCACCCCAAGAGGTCTGTACTCCTTCAGTGGGCCCTCAGATTCCCGC
CCTCCTTGGGATTGGGCAGACTCTCGGTCTCCCATAGAGAGATTTTTTT
TCAGCTTAGGGTGTGCAAGGTTTACAGTCAGCTGGGCAAAGTGCAGGGA
AGTAGCTGG

In some embodiments, the intronic domain that promotes trans-splicing primarily in the heart comprises or consists of sequences from the gene ACSS2. In some embodiments, the sequences from gene ACSS2 comprise or consist of:

(SEQ ID NO: 1098)
TGAGAGATGGAGGAGGGTCCTGCCTGACTTCTTTCTGGGGTGGAGGGGC
TAGAGAGAAGCTAGAGGTAATTAAGCCCAGGTTGTGGTGGTGGTGGTGG
GGGTTTCCCTTGGTGGAGTAAAGAGATTGTGAAGGAAAGAAGAGAATGT
GGGCCAGGTGTGGTGGCTCACGCCTGTAATCCTAGCACTTTGGGAGCCC
AAGGCGGGCAGATCACCTGAGGTCAGGAGTTCAAGACCAGCCTGGACAA
CATGGTGAAATCCCATCTCTACTAAAAATACAAAAATTAGCTGGGTGTG
GTGGTGTGCACCTGTAATTCCAGCTACTCGGGAGGCTGAGGCAGGAGAA
TTGCTTGAATCCAGGAGGCAGAGGTTGCAGTCAGCCGAGATTGTGCCAT
TGCACTCCAGCCTGGGCGACAGAGCGAGAGACTCCATCTCAAAACAAAA
ACAACAACAAAAAAAGAGAATGTGAATGATTTTTCCTATTTGCACAGAC
TTTGATATT

In some embodiments, the intronic domain that promotes trans-splicing primarily in the heart comprises or consists of sequences from the gene LBP. In some embodiments, the sequences from gene LBP comprise or consist of:

(SEQ ID NO: 1099)
CCAAGGCAGAAAATCGCTTGAGCTCAGGAGTTCAAGACCAGCCTGGGCA
ACGTGGTAAAACCTGTCTCCACAAAAAAATACAAAAATTAGCCAGGCAT
GATGGCACATGCCTGTAGTCTCAGCTACTTCAGGGGCTGAGTTGGGAGG
ATGGCTTCAGGCCAGGCGACAGAGGTTGCAGTGAGCTGAGATTGTGCCA
CTGCACTCCAGCCTGGGTGACAAGGTGAGATCCTGTCCCCCACCCCCCC
GCAAAAGAAAAGAAAAATGTGGATTCTCAAGCCGCTTCCCCAAATCTGC
TGAATGTGATCATGCGATTGGGTGCCGCAGGCACTGGGAAGCCAGACAC
ACCGAATCAGGCTGGATGTGCTGGGACACAGCAGGGCGCCGGGAAGGGG
TGGAGAGGGCTCCCTTTGTGGACTGGCCTGACCAGGGACCAGGGCCTGG
CTTTCCCAGGCACCCGGCCATCCCCAAGTTCAGTGGCAGACTGTGCTTC
TCTCCCCAG

In some embodiments, the intronic domain that promotes trans-splicing primarily in the heart comprises or consists of sequences from the gene LBP. In some embodiments, the sequences from gene LBP comprise or consist of:

(SEQ ID NO: 1100)
TAAGAAGGTCCAAGCCTCTGGCCTCCCCCGAGCTTGGCGAGGGCTGAAT
GGAAGACCTCACTGACCAATGGCCCTGGACCAGGCCATGGGGGCTGGTT
TAGACGTACTTGGGCCCAAAGCCCAGGGAGGACAAATCATTTGCCCAGG
TCATGGAGCTGTTGTGGGGCACGCTGGGGACAAAGCTCCAAGTCCGTTC
GGTTGGGCAGAAAATGGTCAACTGCTTGACCAAGGGTTAAACAGCAGGG
GCTAAACAGAGACTCCAGCCCCTCCCTCTGCTCCACCTTGCTCAGTGCC
CCACACTGCCCTGCATTCCACCAAGGTCCGAAAGGAAACCATTCCTCAG
GCGCCTGCAAGAGACCAGGCAGGAAAGCTCAGCGAGGGCGTGGGCCAGG
TGGGAACTGGGCGAGCTCATGAGCACCAGCCCTGGATGGGGAGGCAGCA
ACACTGTCAGACTGGCCTGTTAAAAAGGAGTATGTGAAATCTTCAGAGT
GTATCATCT

In some embodiments, the intronic domain that promotes trans-splicing primarily in the heart comprises or consists of sequences from the gene PABPC1L. In some embodiments, the sequences from gene PABPC1L comprise or consist of:

(SEQ ID NO: 1101)
AATCCCTCCATGCCAAGGTAAGCAGGAGCCTGGGCAGCAGGGAGCCCGA
GGGGGCAGGAGGAGAGCTAACGACAAGGGCTCTCCTAGTGGAGCCAGTG
GATAGCTGTTTGGCCAGATAAAGTGTTTCTTCTTGCTAATACCTGAAGG
CCTGCTGAATCTTAAGAGTGGAAGGGGCCTTAGGTAAGGGTTTGCTACT
TCAGCCCTTCCGTAGACTAATTCTGTGTTGTTCTGTAGGTAGGTCTTTT
GGTACAAGGAAGTGTGTTGTGACTCAGTGTAAGGGTTAAGATTTTTCCA
GCTGTCCAGCAATGTACCTCTGTGTCAAAGGTGACAGACAGGGCCATGG
GATCTTGCAAGCAGTGGTGGATGGGAGGGTTCCACAGAAACCCTGTGGA
TGGAGGAGGATCCTGTATCCAGGATGGTGAGCGAGGGGGAGATCAAGGC
TCTTCTGTGACCTGCTAAGATAGCTGCACTAAGCCCCCCCGCCACTCAT
GTCTCACAG

In some embodiments, the intronic domain that promotes trans-splicing primarily in the heart comprises or consists of sequences from the gene PABPC1L. In some embodiments, the sequences from gene PABPC1L comprise or consist of:

(SEQ ID NO: 1102)
TGGGTGGAATGGTGACAGAAGCAGCTGAGCCCGGGAGAAGCTGTA
AGGAAATAGGCAGGGACTGACTTTCACAAACACTGAGAATTGCGC
CGTGTTCCTGGCTTTCTCTGAAGCACCAAAGAGTTTGGAGGAAGT
TGTGAGAATCAGAGGAGATGATTAACTGTGATCGGAATGGGGGGT
GGTGGAGGGTGAAGGAGGTGCCCTCTTGCCAGCTGCCCTGCCTGG
CTTCATGCAGAATAGTTTGGGCTTTCCCAGGGCCCTGCCTGGCTC
CATTCTGCCTGCCTGAAGGCCTGGCCAGGATGTAACCACCTTCTT
TATTGAAGACTGGGTGTCTGCCATACTTTAAAAACACTTATTTTT
ACCTTTTTGTCCTCAGAAAAGGAAATCCTCGCTTCCATGGCTGCC
AAAAGGACAGTGTTTCTGGCTCTCAGCCCTAAGGCCCTGCAAACT
CTAACTTATTTCCCAATTAGTCTGTATCTATACTTGGGCTCTGTA
TGTG

In some embodiments, the intronic domain that promotes trans-splicing primarily in the heart comprises or consists of sequences from the gene UBE2V1. In some embodiments, the sequences from gene UBE2V1 comprise or consist of:

(SEQ ID NO: 1103)
GTATGCTTGCAGGTTTTGCTGTTTCCTTTCAAATACCATATGTAG
TACTATTTAATATTTTTCTTGTAATCAACTGACTCTTTTGCTTAC
ATTTATTTTAAAGGAAACTTTTACCATTATTATAAATGGGAAGTT
AGTATATTACTTACCCTAAATAAGTTATTCTTAAACATAAATACG
TGACTGTAAAACGAAGTGTTCAATCACATACCACCGAAAGTCCTC
TAACATACCACTACCAACTCTGTCCTTCAGTTGCTTGGACCAAAA
ACCTGGGAGCTGTAGTTGACTCTTGTCTCTCACACCTCACAGGCA
ACCTGATAGCAAATCTCCACAGCTCAGACCTCTGAAATGCCTAGA
ATTACGCCACTCCTCACTACTTTCCTGCTATCACCCTGGTCCAAA
CGGGTATTATCTTTTGCTAGGATTATTGCAGTAGCCTCCTAGCTG
CTTCTGCCCTTGCTCCCTTCACTCTCATCCTCACACAGCAGTCTG
AGGG

In some embodiments, the intronic domain that promotes trans-splicing primarily in the heart comprises or consists of sequences from the gene UBE2V1. In some embodiments, the sequences from gene UBE2V1 comprise or consist of:

(SEQ ID NO: 1104)
AACAGCTGTTAACATCTTGTTCTGTATCCTTACCAACTCTATAAA
GCTCCTTAAGATGTCACTTATCATCTATATGAGTTTGCTAGTGCT
GTCATAACAAAATACCATAGACTGGGTGGGGCTTAAACAACAAAT
TTCTCACAGCTCTAGAGACCAGAAGCCCAAGATCAAGGTGTCAGC
AGGTTTTGTCACATTCTGAGGTACTGGGAGTTAGGACTTAAGTAT
ATAAATTTGAGGGGAACACAGTTCAGCCCAGCCCATAGCACGATC
TGTGAACAAGTACCTCAGTCATCAGCCTTCCTATCAATATGATTC
TTTTGATGTTCATTTTTATTAGTGCAGGCTTGTAAATTAACCAGT
AGTAATGTAGTGACTTTTCCCCCGCTAGCCTAATTAGGATTATTA
AAGATTTAGATTGAACCCTTTCTGAACAATAGTTGTGTTGTCTGG
CTTTTTGCCTATAATGTCACCACTATCTTTCAGTTTTATGTTTGC
TACA

Intronic Domains that Promote Trans-Splicing Primarily in Muscle Tissue

In some embodiments of the compositions of the disclosure, there may be an intronic domain that promotes trans-splicing primarily in muscle tissue.

In some embodiments of the compositions of the disclosure, the intronic domain is derived or isolated from human genes that feature exons that are primarily included in muscle tissue during the process of RNA splicing.

In some embodiments, the intronic domain that promotes trans-splicing primarily in the muscle comprises or consists of sequences from the gene CDK11B. In some embodiments, the sequences from gene CDK11B comprise or consist of:

(SEQ ID NO: 1105)
GTAAGAACCTCCCTGGAGACAGAGCCCCCTCCTTGTCTGTCTCCG
AGTGCAGCAGCCCTCTCCTAAGACAGCCGGTCCTGCGCAGCGTGG
GGCACGCCAGGGTCTGGGAGGCCGCCTGATTCGTTGGCCGCTCCA
GGCTGTGCCGGTGGGGCCCGCATTCCTCAGCGAGGCACAGTGCTG
ACCAAGGGCGTCCTGAGTGCCCAGCAGGGCCGAGGAGCTGGGAAG
CCCAGGACCAGTTCCCCACAGGGGAGGCCCGTCAGATGGTGGCTC
CGGGCCTGTCCCTGCAGAGCAGAGGCCTGGCCCAGCCTCCGACAG
GCAGTGAGGTTATGTCCTCAGCCTGGTGGACGTGGATGTGGGCTT
GTCCCCCACATGACACCCGACGCCTCCTCCCAGCTCAGCATCCGG
GCTCTGTCTCAGCCGAGCCTGCCAGCACTCTTAGGCCTCGGTGCC
CTGTGTCCAGGGGACGAGAGCCCAGCGCCAGCACCTCCCAGCGGG
ACAC

In some embodiments, the intronic domain that promotes trans-splicing primarily in the muscle comprises or consists of sequences from the gene CDK11B. In some embodiments, the sequences from gene CDK11B comprise or consist of:

(SEQ ID NO: 1106)
TTAGAAACCGCAGAGAAGTAGCTGGCGGGCGGGGGAGTTGAAACT
CCCTTCTATCTAGGACTCTCTCGGGACACCTCCACATCGATTCTT
TTTGTTTGTTTTTTTGAGATGGAATCTTGATCTGTTGCCCAGGCT
GGAGTGCAGTGGCATGATCTCGGCTCACTGCAACCTCTGCCTCCC
GGGTTCAAGTGATCCTCCCGCCTCAGCCTCCCAAGTAGCTGGGAG
TACAGGTGTGCGCCAGCACGCCCGGATAATTTTTTTATTTTTAGT
CGAGACGGGGTTTCACCACGTTGGCCAGGATGGTCTCGAACTCCT
GACCTCAGGTGATCCACCCGCCTCGGCCTCCCAAAGCGCTGGGAT
TACAGGCGTGAGCCACCGCGCCCGGCCTGCATATCCATTCTTGGG
AACACTTGTTGCTTAGCTGAAGCGGAGCCCGCATCCTGCCTGTGG
CAGCACTCGCCCCGGTGCTGGTCTGAGCAGACGCCTCCTTTCTCT
TGCA

In some embodiments, the intronic domain that promotes trans-splicing primarily in the muscle comprises or consists of sequences from the gene HP1BP3. In some embodiments, the sequences from gene HP1BP3 comprise or consist of:

(SEQ ID NO: 1107)
GTGAGTGTGCGGAGCGCCGAGGCTCGCGTTCCGCCGCTGCTTCCG
TCGCCGGGGCGGGCGCCGCGGTGTCCCTGCCGAGCGTGAGCCCCG
AACTGTCACCGCGCCGAGCCCCAGCCTCATCCGTCGGTGTCCGCG
GTCCGAGCTTTCCCCGCCTCGCTGGCGGCCCGCGCTCTCCCCCGC
CGTGCGCGTTCGCCGCTGTGGCCTGTAGCTACCTCGCCTCGCTGC
TTTGTCTCCGCTGCTCTTGCCGTCCTTCCCGCGGCCTTCTGCGGG
CGGGGCTGTCGGCTGTCGCTTTGCTGCTCCGCGACGGCGGCCTTG
AGTCTCGCTCCCCCGGTGGGAAACGTAGAACTGGTTACTCGCCAC
CTTAGGCGGTGGGGTTTGAATGAGAGCGGTGCCTCGGTAGGTCGT
CAGAAAGTTACAGTCGCCCATCTGAGCGTTTGTAAGAGATGGAGG
CAGGAGTGGTGAGGTGAGAGCCCCAGATCTGAGGGTTGGTACTGC
ATTA

In some embodiments, the intronic domain that promotes trans-splicing primarily in the muscle comprises or consists of sequences from the gene HP1BP3. In some embodiments, the sequences from gene HP1BP3 comprise or consist of:

(SEQ ID NO: 1108)
CAGGACCGAACAGGCGGCCGGGGAGTGGGCGCGGGCTCGCTGGGC
CCCGAAGGCGAGTGGCGGAGGCCGCTCGCCCGGGGCGCCGACGCC
GCTCCCAGAGAGGAGGGGGCGGGGCGCCCGGGTCCCCGGAGGTGG
CGCCTTTTGTCTGCCTTACCCGGCCGCTCCGCCCCGCTCCCTTCC
TCTCTCCTGCGACCTAGCCCCCTGCCCTTTTGGTGGACTCCTCCT
CAGGGGCCCTCCGGCGCGCGCGCTCCCGCCCTTGTTTTGTTTGGA
TTTGGGGGAGGAGGGGGAGCTCTTCTCTGGCCGTGCGACCCTGCG
TCCCAGCGCGCCTGCGTCGGCGTCCTCTGCCCGCCCCGATTGGCT
GAGGGCTGCGCATGCTCCGAGGCCCGCCGCGGTCCGGCGTCCGCC
CGCGCCCTCCCCTCACCCCACTCCCTCAGCGGTTTTCGGAGCCGG
TTCGAAAGATGCCGGCGGCGGTGACTGGCTGCGGCGGGCCCGCCC
CCCG

In some embodiments, the intronic domain that promotes trans-splicing primarily in the muscle comprises or consists of sequences from the gene FNDC5. In some embodiments, the sequences from gene FNDC5 comprise or consist of:

(SEQ ID NO: 1109)
TCCCTCAGCCCCAGTGAACGTCACCGTCAGGCACCTCAAGGCCAAC
TCTGCAGTGGTGAGCTGGGATGTTCTGGAGGATGAGGTTGTCATC
GGATTTGCCATCTCCCAGCAGGTAATGCTCCACTGTCCTTAGGAG
GGAAGGACACATCAAGACAGTAGTGGCAGATGGGGGTAGGAGTGT
GGCAGTGGTGGTGATGGAGAAATAGAAAGGAAATAGGAACTTGGG
GCAAGGGAGATGAACAGCAATTTGGACATATGTCTGAAAATTGGG
GCCCCATTCAGACATATGTCTGAAAATTCTCTAGAAGAGAATCAG
TCAGTGAGGGAGGGTTTGGGAACTTTCAAGTGGAACACAGAAATT
AAGGTTTTGATCTCAGAGACATCTGCATTTAGGACCTAGCTCCAG
TGCTTACTAGATGTGTGGCCTTGGGCAACTTCCCCAACCTCTCTG
AACCTGTTTCCTTCCCTGTAAAATGGAGATATTGATAGCGGAGGT
GAT

In some embodiments, the intronic domain that promotes trans-splicing primarily in the muscle comprises or consists of sequences from the gene FNDC5. In some embodiments, the sequences from gene FNDC5 comprise or consist of:

(SEQ ID NO: 1110)
TCCCAGTCCAGGGCTCACCCCAGCCTGACACTTCTCGATCTCTGG
GTCTTTCTCCTTCGCGCAAGGTCACTCATTGGACAGGGCAGTATT
GTCAAAAGAGCACTGGACTGGGGAGTCCTTTTGTCTGCTTTTCAT
CAGCTAACCTTCAGTTTCTGTCCACCTATGTCCCCTACATAACTC
TCTCCACAACTCTAACCACCCATCCCTTAGATAGGGCCCCGGCCC
AGGCCCATCACTTTGAGCCTTGGACCCCTCATGGGGCCGGCCTGT
GGCCAGCAGGGGCTTCCGTCCCAGTCATGAGGAGCCTGGGGCCTC
TTGGGCTTGGTTCTTCAACCCACTGAAGAGCTGAGAGCCATGTCA
CTGGACCCCACTTGAGGCCTTAGTTTCTTCCTTCCCCTCCTGCAT
GGCCTTCCCCAGGTTTCCCCTTCTTACTTATGAGACAGTGGGTCT
GGGCCTCACAATAGGGGATGCTCAGGCTTAGGTGCCTCCCCGTCC
CCCA

In some embodiments, the intronic domain that promotes trans-splicing primarily in the muscle comprises or consists of sequences from the gene RAP1A. In some embodiments, the sequences from gene RAP1A comprise or consist of:

(SEQ ID NO: 1111)
TGCGCGGCTCCTTTAAGCGGACTCCGGAACGCCGTGGGGAAGGCG
GGGCCGAAGCCCAGCCATCGCCAACTTGGAGGGGCGGGGCAGAAC
CAGGGGGCGGGGCCTGCGTGGTGCGTGCGTGCGCGTTCTGGAGGA
GGCGCCGCCGCCGCTCCCGAGGCCCCTGCCGCCGCCGCTCCCGCT
GCTGTCGCCGCGCAGAGCCGGAGCAGGAGCCACGGCCGAGAGGAG
GGAGGAGGAGGAGGAGGAGGTGGAGGAGGTGGAGGAGGTGGAGGA
GGCGCCGGACCGGGGGGGTGAGTAAGGGGCGGGGAGCTCCAGACG
GCCCCAGAGGGAGCGGGGGGCGCGGGTCGGCCGAGCCGAGGGTGA
GGCGGCTCATGTCGCCGGTCAGTCGCCTGGCTCCCCCTTCCTCCT
CCGCGTTCCATCGGTGTCGGGGCGGCGGCCCGGGGGCCTGGCCGG
CGGGAGGGGCCGGAGATCTGGGAGGCCCGGCCGCCGCCAGGGCCA
GAGC

In some embodiments, the intronic domain that promotes trans-splicing primarily in the muscle comprises or consists of sequences from the gene RAP1A. In some embodiments, the sequences from gene RAP1A comprise or consist of:

(SEQ ID NO: 1112)
TTGGACATCGCTGCCTTCCTCCTCTTGCATGTCCCCTGTCTAGGG
TCAGAGCTTCCTTCAAACCCAGCTCTCCCTTTTGGGCCCGCGGGG
GCGGGGGGGAGGGACACACGGGGAACGGGGTTGGAGGGTTGGGTG
AATTCTCTGTCCTCTGCTGTAGGAGTGCGGGTAGACTGGAGCATT
GCTGTCTCCGGGCTCACTTCTGTCAGGGTTCAGGGTCTCCCTTTC
CTTCCTTTATTCCCCCAGTTAATAGGGCTGACATCTACATCCCAC
TCCACCTTCCAGCCCCTTTCCCGCCTTCTTTACCCTCATGTCTTC
CGTAGCTTACCATGTGTTCGCCTTTTCAGTGTTACTTTTCATGGG
TCTCTGCTTCTTCCCTGAATTTTGTTAGGGAAGAGGTCACGGTGT
GATTCTTGTTATCCTTCAAACTAGCGGCTTGGTAAGAGTATCTCT
TGCCCCCTTAATCAGGAAGTTAAGGACGGTGTCTTCAGCCGAGTT
GTGT

In some embodiments, the intronic domain that promotes trans-splicing primarily in the muscle comprises or consists of sequences from the gene TMOD4. In some embodiments, the sequences from gene TMOD4 comprise or consist of:

(SEQ ID NO: 1113)
GTGAGTGTGTTTGGGGAGCATGGTCCCCAGGGCTCTGGGCAGGCA
GTCCTAGTGTCTGGGAGAAGGTAGAAAAGGGTGAAATTGATGATT
CTGGGAGAGGACATGCTGCATGAGTGATAAGAAAACCTGTGGAGT
GCCAGGTGAGGTGGCTCATGCCTGTAATCCCAGCATTTAGGGAGG
CCGAGGCAGGTGGATCACCTGAGGTCAGGAGTTCAAGACCAGCCT
GGCCAACATAGTGAAACCTTGTCTCTACTAAAACTACAAAAATTA
GCTGGTTGCATGCCTGTAATCCCAGATATTATACTTGGGAAGCAG
AGGCACAAGAATTTCTTGAACCCGGGAGGTCAAGGCTGCTGTGAG
TTGAGATTGTGCCACTGCATTCCAGCCTAGGTGGATAGAGCGAGA
CTCCATCTCAAAAAAAAAAAAAAAGAAAAGAAAAGAAAACCAATG
GGGAGGAGATTATGGGGAACATGTTGGGGTTCCCTTCCTGCCCTC
ATTC

In some embodiments, the intronic domain that promotes trans-splicing primarily in the muscle comprises or consists of sequences from the gene TMOD4. In some embodiments, the sequences from gene TMOD4 comprise or consist of:

(SEQ ID NO: 1114)
AGAGCGAGACTCCGTCTCAAAAAACAAAAAACAAAAACAAAAACA
ACAACAAAAAAAATTAGCCAGGCATGGCAGCATGCACCTGTAATC
CCAGTTACTTGGGAGGCTGAGGCAGAAGAATTGCTTGAACCCGGG
AGGTGGAGGTGGCAGTGAGCCAAGATCGCACCACTGCACTCCAGC
CTGGGGGACAGAGTGAGACTCCGTCTCGGGGGGAAAAAAAGTGGA
ACCATTCTGGCTGAAAGAGGAGAGTCTCTACAGACTCCTTTATCC
TCTAGTGGCTCATAAGGGACCTTCCCTTAGCCCTTTCTCAATGTT
TTAGAAACTACTGCCTTAAGAAGTTTATGGAAAGGACCCCTGGGG
CTGAAAGAGGGTGGGAAACTTCTTTGGCAAAGTGTAAGTGGTCAG
GCTTTACAAATGCAACAGATTCAGAAATAGGTAGGAGGGAGGCTT
GGAACAGGTTCCTTCTCATTCCCTGTGACTCCTCTTCTTACCCTC
TCCA

In some embodiments, the intronic domain that promotes trans-splicing primarily in the muscle comprises or consists of sequences from the gene DAP3. In some embodiments, the sequences from gene DAP3 comprise or consist of:

(SEQ ID NO: 1115)
GTTTTTGTTTTTTTTTTTTTTTTTTTGAGACAGAGTCTCACTCTG
TCACCTGGGCTAGCGTACAGTGGTGTGATATCAGCTCACTGCAAC
CTCTGGATGCAAGCAATCCTCCTGCCTTAGCCTCCAGAGTAGCTG
GGACTACAGGTGCATATCACCACTCCTGACTAATTTTTTTTTTTT
TTTTTTTTTGAGACACAGTCTTGCTCTGTCGCCCAGGCTGGAGTG
CAGTGGCATGATCTCAGCTCACTGTAAGCTCCGCCTCCCAGTTTC
ATGCCATTCTCCTGCCTCAGCCTCCCAAGTAGCTGGGACTACAGG
CGCCCACCACCACACCTAGCTATTTTTTTGCATTTTTAGTAGAGA
CGGGTTTTCACCGTGTTAGCCAGGACCGTCTCGATCTGACCTCGT
GATCTGCCCGCCTCGGCCTCCCAAAGTGCTGGGATTACAGGCGTG
AGCCACTGCGCGTGGCCTCCGACTAATTTTTTAAAATATTTTTTG
GTAG

In some embodiments, the intronic domain that promotes trans-splicing primarily in the muscle comprises or consists of sequences from the gene DAP3. In some embodiments, the sequences from gene DAP3 comprise or consist of:

(SEQ ID NO: 1116)
TATGAGCCATTACACCTGGCCTATTTTCTTTTTTTTTTTTGAGGT
GGTCTCACTCTGTCATCCATGCTGGAATGTAGTGCATCTCAGCTC
ACTGTGACCTCCACCTCCTCCCAAGCTCAAGTGATCCTCCTGCCT
CGGCCTTCCGAGGAGCTGGGACTACAGGCATGCACTACCATGCCC
TGCTAATTTTTATATTTTTTGTAAAGACAGGGTCTCACTTTGTTG
CTGAGGCCAGTCTTGAACTCCTGGGCTCAAGCAATCCACCCACCT
CAGCCCCGAGAAGTGTTCCTCCCAAAGTGTGGGGATTGTACATGT
GAGCCACTGTGTCCCGCCTGACTTTTTTATTTTCCCAGTATGATG
ATTGTGAATGGTATCTGGTTTTCATCATTTTAATTAGCATTTCCC
TGATTTGTAATGAGGTTCAAAAATCTTTTCATGGGTTCACTAGCC
TTTTGTGTTTCGTTTTCTGTGAAATGCCTGTTCATGCCTTTTGCT
CATT

In some embodiments, the intronic domain that promotes trans-splicing primarily in the muscle comprises or consists of sequences from the gene TNNI1. In some embodiments, the sequences from gene TNNI1 comprise or consist of:

(SEQ ID NO: 1117)
GTAAGCGTCTGCAGCCCAGAACTTCAGATGTAGGTTGATCCCAGG
CAGAGAACCTGGGGCTTTTGTTTAAGGAGAAAGCGAGGTCTTGGA
GCTGGAGAAAACCATTTCTTGCCTGTGCAGTTGGTTTGTTGGTAG
AACATTCCTAAAATACTCCCACAGCATTTTGTCCCACAGAATGGC
ATCTCTCCTGCAAAACTGTTGTGAGAGGTGAAACTTTTTCCTGGC
CATTTGGAGGCTGTGAGCAAGGAGCAGAATCTTCCGTGAGGTTCC
TTAGGAGCCCCAGGGCAGAGCCTAAGGGATGATGTTTTCACACAG
GATGCCCAGCAGAGTGTGGGAGGGAGAGCGCACCCAGGGGAAGGG
GTGGCCCTGGGATCAGAGAGCCAGGGTGAGTGCTCAAAGTCAGCA
ATGCCTGCAGCGGAAGAGAAGGAGGGTGCCTGACGCGCAGGACTC
CCAGGGAGCCTAGAAGGCAGGGCCAGAGCTCCCTGCTGGCCAAGG
AAAC

In some embodiments, the intronic domain that promotes trans-splicing primarily in the muscle comprises or consists of sequences from the gene TNNI1. In some embodiments, the sequences from gene TNNI1 comprise or consist of:

(SEQ ID NO: 1118)
CTCTTGGGCTTTTGGGGCATGGGTTCAGGGGCCTCATTTCCATAT
TGCCTTATAAGAGATGCAGGGTTAGCCCAATGGTCCTCTTCCCCC
AGCTGCTACTTGCCCCTCTGGGCCTTCACTGTGGCCCTTTGCTCT
CCCTCATTCCCCCTCCCAGTCCCATCTCTGCTCAGTCCCCACACC
TGGTCTGGCTGCTCATTCTTTCCCTTTCTGCAGCTCGAGTCTCCC
CAGGTGGGTGCTGGTTTCACTCAGTTGGTGGCACCTATGTGCACA
GAATTTGCCTCTGCTCCTGAGCCACAAATTCACATGGCTTTCCGC
CTATGTGCCTGTTGTGTGCATTGCAGCACGCATCTGCCCTGTGAG
GTGTGTGTGTGTGTGTGTGTGTGTGTGTGTGTGTGTGTGTGTGTG
AAGGGTGGGAGGGAGGAGGGGCAGCAGGAGGGGGCAGTGGGTCTG
TTCTATTTTTACCAGCCAGTTGCTGCTGGACACAGTTTTCATAGC
CTCC

In some embodiments, the intronic domain that promotes trans-splicing primarily in the muscle comprises or consists of sequences from the gene OBSCN. In some embodiments, the sequences from gene OBSCN comprise or consist of:

(SEQ ID NO: 1119)
TCCCAAGGGCCCACCTCTTCATCCATAGGGGGTTAGTATTTCAGC
ATATGGTCTTTGGGGGGCATCTCAACATTTAGTCCATAAGAGAGG
GTAAGTCCAGCCTCAGTCACTCCATCTTGACTAGAAACACTAGTC
CTAGCTTCTGGTTTTGAGGCCTGTATCTTTTCATCCCATTATTCC
CTAGTAGGATGGAACTGGAAGGTTCTACCACTATCACTGTTTCTT
CTGAGGCCCCTGGATCATTCGGTATTAGTTTGTTGTGTCAAAGAG
GCTTGTAGTTAGTTATAGGCCTTGAAGAATGGGGCTAGTAATAGC
CTGAGGTGGAGAGGCTACAATGTTGCTGTTTCCCCTGTGACTTCC
TAGCTCCCAAGTCTAAGTGGCTCCCGGGCAAGGCTCTGTAGTCAG
GACCTCTGAGGGCCGAGGCCATCCTGGTGCGGTAGCTTATGCTGG
GGCAGATGGGATGGCAGACCTTGCTCCAGCTCCCCTCTTCCTTCC
CCAG

In some embodiments, the intronic domain that promotes trans-splicing primarily in the muscle comprises or consists of sequences from the gene OBSCN. In some embodiments, the sequences from gene OBSCN comprise or consist of:

(SEQ ID NO: 1120)
TAGGGGCTGCCAGAGGTGGGCTGTGCCCCATGGACTGACGTCGCA
GGGCCTCTCGGGGAGTGATGCTGCTGGGTGAGGGCAGGGCTTGGT
CTGACAGCATCCAATCCTGGAGCCTTCCTCCAGGCCCTGGCGGTC
TCTGTCCACTCAGTCAGCAAACAGGGGCTTCCAGCGTCATCCATG
CCCAAGCCCCTCAGCACCCCTCCCGCCCTGCTCCCCTCGATGCCC
TGCTTTATGGCTGACCCCTTTTCTGTTCTGCCAGCCGGGAGCAGG
AGGGAACAGAGTCCACCACTGATGAGGGCCAGCTGCCCCAGGTGG
TGGAGGAGCTGAGAGACCTCCAGGTGGCCCCTGGCACACGCCTGG
CCAAGTTCCAGCTCAAGGTGAAAGGTGAGAAGGAGAGGGAGTGGG
GACACAGCCATGGCAGGATGGAGGCCAAGGAGCCATACCCCATCA
GCCACAATTGAAAGGAGACCCCAGCAGCGGGGTCAGGCAGTCACT
CATC

In some embodiments, the intronic domain that promotes trans-splicing primarily in the muscle comprises or consists of sequences from the gene KANSL3. In some embodiments, the sequences from gene KANSL3 comprise or consist of:

(SEQ ID NO: 1121)
GTGAGAGCAGCGTTGGCTTCGAGGGGAGTGGCGGGCCGTGGGGAT
CGCGGCCGTGGTTGGGACGGCCGGGGGTCTCGGCGAGGGCTTCAG
GGCTCCCGGGGCATGCGGGGGCCCGGGCTAGGGCCGGCTGCCGGT
GCTGGTCGGGCGGGGCGGGTCGGGCCGGGGGCGGGCTGGCAGGGG
CCGCGTCTGGGCCTCCGTGACGCGGACGCCGAGCCCTGCCCCAGG
GAGCGGGAGGCGGGAGGCGGGGGCCAGGCGGCTCCTCCTCGTCGC
GAACTCTGGATGACCTTGGCCAAATCGCTTCAGTTCCCCGAGCTG
GCGACTTCGTCTGTAAAATGCGAGTCATAGCCCTGTCTCCTAGAT
GGGCCGTGTGGAGGCTCAGTGGCGGGAACGCCTGCGGGGCAGCCG
GGGTCCTGGGCGGTTTGCTAGGGACGCCTCACTGGGGGTGAGGGT
GAAAGGGCTTCCACGGGGATCATTTTGTCACAGGCTTTGTTGGGG
GGGC

In some embodiments, the intronic domain that promotes trans-splicing primarily in the muscle comprises or consists of sequences from the gene KANSL3. In some embodiments, the sequences from gene KANSL3 comprise or consist of:

(SEQ ID NO: 1122)
CCTCCCGGGTAGCTGGGACTACAGGCATACACCACTATCACACAC
ACCTAATTTCTAAAATTTTTTGTAGATACGGGGGTCTCATCATAT
TGGCCAGGCTCATCTCAAACTCCTGAGCTCAAGAGGTCCTCCCGC
CTAGGCCTCCCAAAGCGCTGGGATTACAGGCGTGAGCCACATTAG
GTAAATTCTTAATGAGTAAATTCAGGTGGCTGCCCTGGTGGAGGT
GGACCGTTGGGTGCTCGACCGCCCAGAGGGCACAAAGGACCAACA
CGGTGGGGGTGGCGAGAACTGGGGCCGTAGAAGCCAGCCTAGTCC
TCCGCCCTCGCTTTCCCTACTGAATATGCGCGAGGGGGAAACTCC
AGCCTAGTTGTTGACTTTCTGCTTCGGACATGCTCGCGTGTTCTT
GGCTCTCCTCTTCCGGACAGAGGCGCCGGAGCTTCATCCGTTCCG
GGCTTTGCCGCTCACCGCCTCTCCCTTCCCAGGCCGTAACCCGCT
TCCG

In some embodiments, the intronic domain that promotes trans-splicing primarily in the muscle comprises or consists of sequences from the gene NEB. In some embodiments, the sequences from gene NEB comprise or consist of:

(SEQ ID NO: 1123)
GTAGTGCCCACTTGGTCTTTTCTTTCCCTCTTAATCCAAATAATT
GCACTAGTGACACTCATTAATGTGTTCCTCCTACCATTTACATCA
CCACCTAACTGTGGGTTTCAAATTGGGTTCTCATGTGCTTGGTAC
TACCACCTAATCTATTACCCATGTTAATTCTTCTAATTTTTTATT
TGAATATTACCTTAGCATTCTATTAACTTCAAGCTTTCATTTGAT
ATTTAATGTTAACTGATCATGCTTATACCCTGCCTGTATTCATAT
TTTTACTTACTAAACTGTGAACCCTTTAAAAATTGGAATTGTTTT
TTATTTTCTTCTTTGCCTGAGAGAATCAATCTGGGCTTTCTTGTC
AATGTTGTTGCTTTTCTTTCATGATATAGAAAGAGTACAGGAAAG
ATCTAGAGGAAAGCATCCGTGGGAAGGGCCTCACTGAAATGGAAG
ATACACCTGACATGCTAAGAGCAAAGAATGCCACTCAAATCCTCA
ATGA

In some embodiments, the intronic domain that promotes trans-splicing primarily in the muscle comprises or consists of sequences from the gene NEB. In some embodiments, the sequences from gene NEB comprise or consist of:

(SEQ ID NO: 1124)
CCAGCCTGGGCATCATAGCAAGACCCCATATTAAAAAAACAGGTC
ATGATTAGGAAAAAAAGCAAGTTAGCATTTGGTAGCCTTAGGCTC
TCCTTCCACTGCGATTTTTTTCCCCTTGCCTCACTGACTTGTAAT
TCTGAAATAAAGTCTTTTGTCACCTTAAGAAAGGTGTGGGAAAGT
TCAGAAGAGACAACAGAGGATTCAGAGTCCTGATGGTGCATCCAT
CCTTCCACTCTGTGAAGGGCCTGCCCCAGTCACAACATTAGACTG
ATGAGTGAGGGGCCCATCAAAGTGTCTCAGCCTGAATTTCCTTGT
GGCCATTTGCTCCAGACAAAGTATTTCAAGTACAATCATACATGT
TTCAAATGTGTGACCATTAGCAGAACTATGATTAAGAGCCCATAC
ATAGAAAATGCTGTTTTTTTTTTCTTCCTGAGATGGAGAGATATA
TTGTAATAAAGATTTTTTTTTAACTTGTCTTTCCTTCCTACTTAC
ATTA

In some embodiments, the intronic domain that promotes trans-splicing primarily in the muscle comprises or consists of sequences from the gene SEPT2. In some embodiments, the sequences from gene SEPT2 comprise or consist of:

(SEQ ID NO: 1125)
GGGGGACGCGCCGCTCGTCCCATACTCTCGGGCGGTGGTCGGCTG
GCTCTGGGGTGCCGGCGGGGCGGTGTGGGGGAGGGGCGGCCGAGC
CCAGGCTCTTCGGCACTTATCGTGGGCTCCCCCGTCCCGGGCGGT
CCTAGTGCCGGCGACAGTGGCTCCGACACGGGCAGGCCCGGGCGC
GGGAGTGGGGATTCCGGTGACCCCTTGTTGACCCTGCGGGAATTC
CTCTGCACTGTCGTCGGTGCTGGGCCTCGGGGCGTCGAGTGCGGT
CGAGGTCGGGAGGTTTGGTGCTTGGAGGGAGGATACAGGTTTAGG
CCCGACAAACACTTAGAGCTTGTGTGGCCATCTTGTCTTTCTAAC
GGTGCCATTTCCTCCCTGAGGCGTGGAGGACTGGCCAGCCCCCAA
ACCTCCAAGCCCATCGGCTGGATGCCGTGGATAAGCGAGGGGAGA
GCGACTAGGCCCTGTCTGCGGGTACCTTCGGCGAGGAGAGGCGAC
GAAG

In some embodiments, the intronic domain that promotes trans-splicing primarily in the muscle comprises or consists of sequences from the gene SEPT2. In some embodiments, the sequences from gene SEPT2 comprise or consist of:

(SEQ ID NO: 1126)
CACGGACAGGCAGCAGGGGGTAGGGTATAGGGTTGGAGGCCGCCG
AGTTGGCCCGGGGATGAGGAGCAAACCTGTGGCTTCCGTGGTCTC
AGTGGAGTCCCAACTGTGACCTGGCCTACTGGCCCAGACAAACCT
GGCTTGCTTTTTTACCTTTTCGGTCTAATTCTTGTCAGTGCCACC
AGACCTTGTCCTCTCTCCACGGAAACACACGAACCAACTACACTG
AACTTCTTGTAGTTCAAACTTCTGCACCTTTGGCCATTGCCCAAA
CTCTTAAATCTGGCTACTTCCCACCTTAACTGCCTTCTCCTTACC
TAGTCTGTGCTTGTGGGATTCCGCCCAAATTAGATTTCCTCCAGA
CAGGCTTAGGTGTTTACCTGATGTTCCAGATGGTATTTGTCCAGT
GGACTATTGGTGCTGTGAGGGCAGGGCACATGTTGTCACCCATAC
TCATTGCCTCGGTTGGTGTCTAGCACCTAGTAGGTTCTCAAATAC
TTTT

In some embodiments, the intronic domain that promotes trans-splicing primarily in the muscle comprises or consists of sequences from the gene SLC6A6. In some embodiments, the sequences from gene SLC6A6 comprise or consist of:

(SEQ ID NO: 1127)
GTTCCTTGGGACCTCCTTCTGCATTCTAACTCGGGGGTCTGCAAA
CAGTAACAACAATGGTGGTGACAGCAGCCTTGACTTACGGGCTTG
CCATGTAACTGGCACTGGGCTGAATCGCCACTCTTTGAACCTGGG
AAATAGCTCTATGGGGTAGGGCAAAGGAGCCCATTTTATAGATGT
GAAAACTATCTATAAACTGCAGGAGTGATATTTACTTGCCCACTG
TCATTCCAGGGTTGGAATCCAGATCCATCTGCTTCTGAATCCTCT
TAACCCTGATGCTAGGTCGCTGGGAGGTGGGGTGTTTGATTTCTG
GGGCTCCTTGCAGCTCAGTACTGCCTTGAAAGGCACCTTGGATTC
GAGCATAAATGTTCCATTTGCATGTGCAAGGTCCCGTCCCCAGGC
CATCGCCAGGTGGACATAACCCTCGCTGCCTCCTCCAGGAAGCCA
GCTCTGACAGCCCTCCTCTCTTTCCTTGCCACCTCCCTCCCCCTC
ATAG

In some embodiments, the intronic domain that promotes trans-splicing primarily in the muscle comprises or consists of sequences from the gene SLC6A6. In some embodiments, the sequences from gene SLC6A6 comprise or consist of:

(SEQ ID NO: 1128)
CAAGTGTTGCGCCGGCGGGCCTGGTGGACTTTAGAAATGATGATG
ATGATGTTTAAAGAAAAAGAGAAGTAGGGAGCGTGGCTTCCTTGT
GTTGTGAATTAACTTGCTCCCTGACATATGTGTAACTTAGGGACT
GTATGCTGCTGGGATGCCTGAACAGTGGTACCAGTTTTGTGTCTG
GCTTCGCAATTTTTTCCATCCTGGGCTTCATGGCACAAGAGCAAG
GGGTGGACATTGCTGATGTGGCTGAGTCAGGTACGGTGGTATCGG
TGGCAGTGGTGGTGGGCACTGCCACCTAGTGTGTAAGCCAAGTAC
TGCAGGCATGAAGCCAGACCCCAGGGGGCTTTGAGGGGGGACGAG
CCTGGTTTCTAAAATGGACCCCCCCCCCGCCACCAAGATATCCCC
CAAATTTCAAAGAGTACAAAGCCAAATTTTAAACATTTGTATTGC
AATTTGGCCCTGATGGGTGGCAAAAGCATGGGGGGTGGTTAAGTT
GGGG

In some embodiments, the intronic domain that promotes trans-splicing primarily in the muscle comprises or consists of sequences from the gene THOC7. In some embodiments, the sequences from gene THOC7 comprise or consist of:

(SEQ ID NO: 1129)
GTGTGCCGGGAGCGTGGTCGGTGGGCTGGGAACACACGTGGGTGG
CCGAGACGGCTAGAAGCGGTGCAAGGGCGGGTGGGACCAGAGGGA
GTCCGGGTTTGTCTAGGGGCGCGGTGAGGACCCAGGAGCCGCGAA
GGGTGGACAGTGGTGCCGGGCTTAGGGCTGGGGGCCTGGAAGGTT
TGTGGAGGTCTGGGAGGGAGTGGGAGCTCCAGGGACGGGGCGGGA
ATTTCGGGACTCGTCTACAAGGGGTGTCAGGATACAGAGCGTGAC
ACCAGGAGGGGTCCTGGGGCATATTGGGAAGCACTCACAGACAGT
GGAGTCGATGGCAATGAGGCCTTGAACTGCGAGTGGATTGTGGGT
GAGAAGGAGGAGCAGGGAATTATTTATAAAAGGAATAGATAGGGA
TGAGGTATCCGGAGGGTGATCAGGAAGTGTACCTGGGAGATTTAC
GGGGGTAAGGAGGAGCCAGTATAGTGATGAGACCGTCTAGGGTAG
ATCA

In some embodiments, the intronic domain that promotes trans-splicing primarily in the muscle comprises or consists of sequences from the gene THOC7. In some embodiments, the sequences from gene THOC7 comprise or consist of:

(SEQ ID NO: 1130)
GGGGCGGCGGGGCCGCAGTCACCTTCCCGGGGAAGAAGCCGGGAC
CCCGACTGCGGGCGGCCCGCAGCTCCCGCCGCACCCCCGCAGCCC
TGGCGGCGTCCGGGCGAGCTCACCTGGCGGCTGGCTGCGGCCTCC
GGGGCGGCCTCTGGGCGTGGGCCGGGCGGGGGCTGCAAGGGGGGC
GGCGGGAGCCCGGGCTGCAGCCGGGGCCGCCGCCGGCCGCCAAGC
CTCGCCCCAACTCGCGCCGGCCGCGGCCGCCCCCGGGGAGTACCG
GGAGCGCGGGGAGCGCGGGTCCCGCGGCGGCGGCGGCGGCGGCGG
CGGCGCGGCGCGGCGCGGCTCAGGCGTCGGAGCAGGCGGCCGCGG
GCGCCGCCGCCTCCTCCTCCATGGCTGTTTACCCGGCTGCATTGT
GGGAGTTTGACCTCCGCCGCCGCCAACCGCCGCCTCAGCTTGCGC
CGCCGCCGCCGCCGCCGCCGCCGCGCACGCCATGGGAGCCGTGAC
TGAC

In some embodiments, the intronic domain that promotes trans-splicing primarily in the muscle comprises or consists of sequences from the gene MBNL1. In some embodiments, the sequences from gene MBNL1 comprise or consist of:

(SEQ ID NO: 1131)
AACACGTCAGACTCATTTACAAGAATGCTAGATTGTGCATATGTG
GCATGTTAATAAGTAACCTTTGGTAGAACTATTATGATGGATTCA
ATACAATCCATCTATGGTGCAAATCAATGATAAAGAACTCTCATG
CGTCTACCATGTATGACTGTAGAATTGCTGGTGTAAGCAGAACTA
TAGCAGAGCAAATTTCAACATGCCCTTGGCCACCTGGGTTGTAGG
GGACACTTTACTTTTTGCATTGTGCTGTGACCATTTCATGCATAA
TCTTGGATCTTCATACTATTTGACAACCAAAAGAAAGAAAAGGGA
ATGGATATATATATACACACATATATTATATCTACATATACATAC
ATATGTATATGCCTACAGTACATTAGCAATGCTGGAAATAGCTGA
TGCCACAAAATGCAGCCTGCATGCATGCAGAAGTTTATAGCTGGC
CCTTCATCTGCATCGATTGGTGTTGAAGGTCCTTGGTATGTTTCG
ACAG

In some embodiments, the intronic domain that promotes trans-splicing primarily in the muscle comprises or consists of sequences from the gene MBNL1. In some embodiments, the sequences from gene MBNL1 comprise or consist of:

(SEQ ID NO: 1132)
TATGAGAAGCTTCATTATGCTTGGAGCACATTTTCGTGCCATTTG
CCAATGCTGTAAAATTGTGTAAAAATGTCAGCTGAGAACTGGAAA
TAAAACAGGGACATATTAATATACACGCCAGACTGTGGATTGTAG
TATGTTGAATTAGCCTAAGATGTCTAAATTGAAAGAACAGCAACA
AAATGCCTTTGTGTATCTGATTTCATTATTCTCCCAACAACATTG
CTGAAATGGGATTAGTATCATTTTATAGAAGAGACTTAAGCATAG
AGTAATGAGTTGCTTTGCTCAGGGTCACACAGCCAGTAAATGGAT
CTCTGGGGCTAGCACCCGTGTCATCAGTCCTCTGACAGTCTCAAT
CCATTGTGCTGCCTCTCTCAGGTTAGCAGGCATTAATTTCAGGTT
GAAGAATCAAGCTACAAATCCGAAGTTCTTAACTTTGGGAACTAT
TGAAATAAGTCAACTCTATATTGGTTTCTTATTGTATTTTCTCTC
AAGT

In some embodiments, the intronic domain that promotes trans-splicing primarily in the muscle comprises or consists of sequences from the gene SPARCL1. In some embodiments, the sequences from gene SPARCL1 comprise or consist of:

(SEQ ID NO: 1133)
GTACGTACCATCTTACTCCTTAAAATGTTTGGAACTTTTTGTTTA
AAAACTCAAACTCAAAGTGAAATCTCAATTTGTCACAATGGTGGT
TTTTTTGTTTGTTTTGGGATTTTTTTAGTTTTTGTCCCCCTTCCT
TACACCTTTGTGTAAGAGGTAAAATGGAGAATTTGCAAAATAACT
AGAAAAAAGCTGGAGTGGGAGCCAGGAAATCTAGCTTCAATCCCA
GCTCCCCTTCTACAGAGCTCAGTGATCTCCAGCATGTCACACTGT
AGGCTTTCTAGGCCTGTTCTTCATCTGCCACATCCTGGGGTTGTT
GTAAAGATCAAATAAGGTAATATCCCATTTGTAAATTTATTTGGT
AAACCCAAAGCACTATGGCAATAGAAATGTCAAGTATATTTTATA
TGCCATTACCCGTTCTAAATAAAGTAAACACCCATTCCATTGTTA
ATATTCTAAATTTCTCCTTGTTATATGTAGTCATTTTCAGAAGGA
TCAC

In some embodiments, the intronic domain that promotes trans-splicing primarily in the muscle comprises or consists of sequences from the gene SPARCL1. In some embodiments, the sequences from gene SPARCL1 comprise or consist of:

(SEQ ID NO: 1134)
GCATTCACTAGGAGGATAGAATGTTTTAAATTATCATCGCTAATA
GAGTCCACATATTTACTTTAAGCATGAATTACCATATATAAAATA
TTTTATTTTAATACAATAAAAATCATGGAATATATTTTAGTGGGT
TTAGTAGTTTAAGGGGAAAAGATAGCAGCAATTTAGGATTCATAC
AATTAATGGAATTATTTTCAATTTTAGAATCCTAAAATATTAGGG
TTAGAGGATCATCTAGGTCTCACTCTTCATTGTTCATCACTTGAG
AAAACTGTATCTAAGAAAGGGCAAGTGACTTTCCCAAGGTCTCCT
GGCAGTCAGGGACACTGCTAGAGCCAGAATCCAGCTCTCCTGACA
GTTTAATGCTTTTCTTGTTTTAAGGTGTTTTTCAATGTATTAGAG
TTTTGGGGCAGCGGGGGGAGGTTGCTCCATGTTTTTACTTAAATA
CATTTTTCTTTCATGGAAAATTGAATTCCTATGCTTTTTGCCTTG
TTTA

In some embodiments, the intronic domain that promotes trans-splicing primarily in the muscle comprises or consists of sequences from the gene PALLD. In some embodiments, the sequences from gene PALLD comprise or consist of:

(SEQ ID NO: 1135)
AAAAGAAATATACTGCATGATGATTTGTGCTTTTCGATTTAATGT
CACACAATCGTTTTTACACTGTATGTGCTTTCCCTCCCCACAGCC
CCAGTCCCAGCCCCAGTCTCGCAGTTCCATTCCTTCAGGGCAGCT
TCCATTCTACCGTGAGTTTTTATTGCTTTCCCCTAAAACGCTGCC
CTGGAGTCGCGTTTATTGGACACTCTTTAGTCTGTAAAGGAAAAA
AACACAAACACCGGAGGAGAATCATCCTAACAAGCCGACAACATA
TCTATGGGCCTAAAAAACTTAAATTCTTTCCCGTTATTAGCACTT
CTCGATCGTTCCAGGAAAGCCCGATTTGTAGCTTGGCTGCCCCAG
GAAGGGGCCTGCGACCAGAAAGATGCAAAGGGCGGGACAAGGTTA
CGAGTGGGTGCGGGGAAGCCTCCCTAACCTGGGGGCCGCGTCCCA
GAGCTGCGAGCCACGCCTCCTCTCTCCCGCCCGCCGCGCCGCCGG
ACTC

In some embodiments, the intronic domain that promotes trans-splicing primarily in the muscle comprises or consists of sequences from the gene PALLD. In some embodiments, the sequences from gene PALLD comprise or consist of:

(SEQ ID NO: 1136)
TAGGCGCGGGGAATCGGCCCTGAGGCTGGTGGAAGAAATGTGTGA
ATTAAAGGGAGTGCAGCGTGCAGGGTGGGCCGCGAGTCGGGAGTG
CAGGGCTCGGGACAGGGTGGGGGCGGGGAGGACAAGAGACTCGTC
AGCGCACTTTCCACCCGGCCGGTTCCTTCCCGAGCCCTCTCCCTC
CCCCGGCCCGCGGGAGCACAGATCCCTGAAAGCGGAGCTCCAGAT
AGGAATGTCTGCATCATCTTGGCAGGGCCCAGACGGGAGAAGATG
CTCTGCTGCCACGGACGATCCCTCCCGCTCTCCTAACTCTTCTCT
GAACACCTCGCACCCCCACGGCCCCCCGCCCCTCTCTTAATCTTG
GCCACGCTCCCTGAGGGGTCAGACCTGGGGTGGGAGAGCAGAGAA
AGCGGGGCTGGCAGAGACAGGGGTGAGGGCCGGGAGAGGCGATGA
CATCATTCCCTGGCAGCTGGCGGGGAGAGGGTGGGGACGGAGGGG
TCCC

In some embodiments, the intronic domain that promotes trans-splicing primarily in the muscle comprises or consists of sequences from the gene ASB5. In some embodiments, the sequences from gene ASB5 comprise or consist of:

(SEQ ID NO: 1137)
GTATGTTTATGATAATCCTCTCTTGTTTCAAGTGGAGTTTAACTT
TCATCTGATCCACAATTACATGTTTTCTTTTTAGACTGCTTCAAG
GTAAATATTTTATAAACTTTTGTTAAGGGAAATACTTGTAACTCA
CTTCGTGGCTTTATAAAAAGGAAAATTACTTTTTTAATGTAAATG
TTTGGGTTAAAATCAAGGTATCATTGCAAAAGAGATTAAGGAAGG
CATACTATATATATAATAGGAATATATATATGTAATAGAAATATA
TCTATTCCTATTAAGAAACATTTTAAAAAGTTAAACATTTGATCT
GCCAAGTCATAGTATTAGAGATTAGTTTTAGAAGTAATTGATTGC
ACTTCAGTCGTCCTCTAATGTGTACCTGGATTGTATAATTTTAAA
TTAAATCTTTAGTTATGTGTAAAATAATTGTTATTAAAAAGAGGG
GATTTCAAGATTTGGTTTCTCACAACTTTAATAAGATAACCATTT
GACT

In some embodiments, the intronic domain that promotes trans-splicing primarily in the muscle comprises or consists of sequences from the gene ASB5. In some embodiments, the sequences from gene ASB5 comprise or consist of:

(SEQ ID NO: 1138)
CCATTGGATGAAGGAACCGTGTTAAGTTATTAATCTGTGATGTTC
CAGTGTAATGACTTGTAAGAAAAATGTTTTTTTAAATCAGTAAAA
ATAACATAATTGGTTAATTTGATGAGTAGTCATTAAAATACAAGC
CATGGACTGTGTACTGATTTAGCTCATGGTCTACAAATAGTACAG
TGCCATTTTAAAGCCTTAAATGATAGACAACTTTACTCGATATGA
ATATCATTTACATTAACAATGGGAACCCCATACACTGTCTTCAAA
TTGTTCTTGATAACTAAATATATAAAATGGAAAGGATATGCATAT
ATATTTCAATATCTGGATTTTGAAATTTGGAGCACAGAGGTGGAG
TGTCATTTTATATTGAGAATGGAAAGTCAGGAGACTTGTGACACG
TGATGCAATTGCTTCCATGGAATATTTTCAGACCAATAGGAGTTT
GCGAAGAACTTTTATGGAGTGTATAAATATTTTGAGCTGTAGGCA
TCAC

In some embodiments, the intronic domain that promotes trans-splicing primarily in the muscle comprises or consists of sequences from the gene CMYA5. In some embodiments, the sequences from gene CMYA5 comprise or consist of:

(SEQ ID NO: 1139)
AATATTACTCATTTTTTAAAATTGGAAAATATAGAAAAGCACACA
AAATATCACTCAGATAAATACTGATTACAGTTTTGATCTCTATCC
TTCATTGAAAACATTTAAGTCATTTTTCCTGCTTTAGAGAATTCT
ATTCTGGAAGCTAAGCCATTGGTTGATGAATAGAGAGAAGCAGAG
CATAAGCAACTCATTGCCAGTCCCGCTGATCACAGAGACAGAGCC
AACTGCAAATGAAACCATCCTCCATCCCTGTCCTGACACTGCCCA
TCCCACCCATTCCCCCTCCCCTGCACCCCGCCTTGTGAGCACTTA
TGTCCTTGCTGCCCAGACTGAGACAAAAACAACCTCAGGGAAGCT
GAGGGGTTATGATTTTCAAACGTGGGTAATCTTAATGCTCCTGGT
TGTTTTTTCTTTCTGTTGTTAATTAGCTTCTCTCTCTCTTTCTCT
CTCTCTTTTTCTCTCTTCTCCTCCTCCTTCCTCTCTCTTTCTCCC
TAAG

In some embodiments, the intronic domain that promotes trans-splicing primarily in the muscle comprises or consists of sequences from the gene CMYA5. In some embodiments, the sequences from gene CMYA5 comprise or consist of:

(SEQ ID NO: 1140)
TATATAAAACATGATACAATGTAGTGGCAAACAGCATGACTGGAT
GCTTGCTTTTTAATATTTTGGTGCTTTATGGAAAATGAGCTGCCA
ATTAAAAAATGCATTTAGTGGGCTACAGTATTTTCACTTTTAGAC
ACAGTGCTTTTAAAAGCTAAATGACAGCTTTGAAAGAAAACTGCA
TGTGCTTTAGTTAAATAACAATTATCATAATGTAGATTTTTAACT
GCTTCCTCAAGATGTATTCTGCATTTTCAGCCTAATCTAGGCCAT
CTTTCCTGTCACTGCATATAGAACATTTTGTAGATGCTTAGATGC
ATTGGTTAAACATGAGTATTTGAAAGAATCATTCATATCTGCCTT
GAGTAATTTTATGATTTATGTAATTTTAATGAACTGTTCTATCAT
TTTCTCTTCAAAAGATAGTCATGCATTTATCTTCTCTTTCTGCTC
AGCTAACCTAAATGAATCTCCCTCCTTTGATTTTGTTTTGTTTTG
TTGG

In some embodiments, the intronic domain that promotes trans-splicing primarily in the muscle comprises or consists of sequences from the gene MEF2C. In some embodiments, the sequences from gene MEF2C comprise or consist of:

(SEQ ID NO: 1141)
GTGAGTGCTCACATTTGTTTTTATCATTTACCACAGAGTTACCAT
AAAGGTAAAACTGCCAAACTCCTTGGCCTTAGGTATTTACAGAAT
AGACGTGTATAACAATAATAAAGAGTTAAATGACCACCATACACT
TTATATTATAAGCAATGACATATCATTTTGCTTAAAGATCATGTC
ATCTTTCAGGGCCTACTTTTGCAAGTACTGTATCTTATAAAATTA
ATCTATTTCATGGGAAGTCCTGTGTATCATCAATACTATAATTAG
ACAAAGATTAAAATATGTAATGCTATTGGAATTTGTGTTTCACTG
CTTTTTAGATTTTTCTAAACCACCATGTTCACTTATCATGTTTGT
GTTAAAAGTATTCTGGCTGAGGAACAAATACTATACAGTTGAATA
ATCTTAAGGGGTACATTAATCTATAGCCTTTCTTCATCTCAGGGT
GGGTTGCTGTGGCAACCGCCCCAGTTCACTTTAAAATAATTTTTA
AAGC

In some embodiments, the intronic domain that promotes trans-splicing primarily in the muscle comprises or consists of sequences from the gene MEF2C. In some embodiments, the sequences from gene MEF2C comprise or consist of:

(SEQ ID NO: 1142)
GGCCTTTAGGCAATGCATAAAATAAGCCCTTTCAGTAACCTCAAC
TGCATTGCAAACATTTCCCGATTTGACAAATTGTGTTAGCTTCGG
ATATAATGGATTTGACATTCTTCTGTAATTCTCTATCAGATATAA
AACTGAAGGTACAGAAATACAGATAAACATAGAATCCTTAGATAT
GTGTAAACCTTGTCTTGAAATAACAAATCCTTTGTATATTGCAAA
TTCTCAATCACAGACATTCCACATGCTGTATATTCTTCTTGGGTT
AATGCTGCACCTAGTGAATTTTTGAAATTTAGATATTTGGCACTA
TTTATGTTAAAGATTATTATCACAATGCATGACAATTTCAGTTGT
CTTATATTCAAAACACTTTGGTTTAAAATCAGAATATTGTTACTT
CAGATTAGAAGTTTTAGAAATAAATCTATTTGAAAGTATTTTCAT
ATCAGCATTAACAAAATACTATATACTGTTTTGCACTTCCACATT
TGAA

In some embodiments, the intronic domain that promotes trans-splicing primarily in the muscle comprises or consists of sequences from the gene CSNK1A1. In some embodiments, the sequences from gene CSNK1A1 comprise or consist of:

(SEQ ID NO: 1143)
GAAGTGGCAGTGAAGCTAGAATCTCAGAAGGCCAGGCATCCCCAG
TTGCTGTACGAGAGCAAGCTCTATAAGATTCTTCAAGGTGGGGTT
GGCATCCCCCACATACGGTAAGAACCCGGTGCATATAGGCGATCC
GCTGAGGGAAGCACGGAATGGGGAAGTGGGTAGAGGCAGGTAATG
AGACCAGCTGAATCCGGGTCTTGGTGTTTTAGTTACCTGGGTTCC
CTATAGCTGGTGGTTTCCTGTAAGTCAGTCATCTCAATTACTGAG
TAAATTTCAGTTTGCCAACTTATTTCATTGTATTACACTGCTCCA
TTTCGCTTCCGTCATTCTCACTCTTCCCGAGGCTTTCATTTGCAG
TTATGCGTGCAGGTCATCTTTGATCCCTACGCGGAAGGGTTGGCT
GCTGCCTTTTTTACGGCGGCACTTCTGATGTACTTAGGTTTGTCC
AGTGAAGTGGATTTGAAGGTGCCGAAGCGACCCGGCAGTGTCAGT
TCGA

In some embodiments, the intronic domain that promotes trans-splicing primarily in the muscle comprises or consists of sequences from the gene CSNK1A1. In some embodiments, the sequences from gene CSNK1A1 comprise or consist of:

(SEQ ID NO: 1144)
GGGGTGGGGTTCACGATAACGCTGCGCTTTTACTCATTCCCCCCA
CCACCACCGTGCACCCCCAAAGTGCTCGGGCCTCTCCTCATCGCA
CGCACTCGCCAGGGAGTCCAGGGAGCTAGTTTTCCTGGCTTACGA
ACCTCGTCCGCTGTCTCCCTAAAAGGGTCAAGAACCCGATAGAAA
GGTTGGGGGAACTAGTTTCCCACCTTGTTAAACACGTCCCATAAA
TGTCCCTTAGTCGAGCTTTTTTTTTTTTTAACCGTCTTTTAAGGT
GGAAACGGCAAAATCCTCTTTCAGTGATTCCCATGTTTTTTTTTT
TTTAATTCTTAATTCTACTGGGCCTCTTGTTCCAATTAGAACAGT
TTCCGATCGCCTAGTTCCTTAAAAACCTCTCACTACGTTCAGAGG
TTGCCTCTTTTTTCTGGATGAATCATTTTCCTGCACTTTAGAGGA
CGTACGGATTTCGTCACTACCCCCTGAAGAGGCCTTTGAGGCTGG
TTGC

In some embodiments, the intronic domain that promotes trans-splicing primarily in the muscle comprises or consists of sequences from the gene TRDN. In some embodiments, the sequences from gene TRDN comprise or consist of:

(SEQ ID NO: 1145)
GTAACAGAGTTAATTTAATATAATATTACAAATTTATTAGGAAAT
ATATATTATTGAGAACACAGTACATTAGCTGCATCCCCTTTGCAA
ATATGTAAACCAGAACTGCAGAGGTCACACACATATTTTTAAAAA
CTTGTGTTTATTTGTTTGTCTCTAAAATTATTTCTTCTCATTATT
ACCAAGAAAGTGCTCTGATTACAATTCCCAAAAATCCCAAAGAGA
AAAGAAAGAAAAATGAAAAAAGGTTTTTTTAATGAAAAAGATAAA
AAATATAGAAGGGAAGTAAAGAATATTAAGAAGCACTTGTCCCCA
AATTCTCTGATTCTCTCCTTTCTATTCCTAAAATATATATTTAGT
GCATAATATATCCCAAGCACTTGGTAAACATTGGAGATGATGATG
ACAACAAAAATGGCAAAGCTACAAATAGGAAGTAATATTTGGTAA
TTAGAACTCTGCATTCTGATATTTTCAAATGCTCTGAGGCATATT
CTGC

In some embodiments, the intronic domain that promotes trans-splicing primarily in the muscle comprises or consists of sequences from the gene TRDN. In some embodiments, the sequences from gene TRDN comprise or consist of:

(SEQ ID NO: 1146)
TACTGGAGCCAGTACTTATGTGCTTGAGAGCATAGATATAGTGGT
TCAAATCCAAGGTCTCCTATTTAAAGATCTTGGAAAGTTATTTAA
TCAATTTATGTGAAAAGTAGAAATAATAACAGAACCTATATACCT
CATGGAGTTGTGAGCACTGAATGAGAAAATGGTAGAGAATATTGC
CTGACACGTAGTAAGTAGACAATGTTAGCTATCATTCTTATATCT
TTCTTCTTCAAAGCCCATGTTAGTTTACATGTACCTTTTTTTGTA
TATATGGCATAGTCTGTACTAGTGATAGAAATTCATGGGCTTATT
TTCTATTGTTTACATATTTAGTCCAATGTCTTATAGACAACATAT
GCCTGGAGACATTTGTATTGTGAATATCAACTCAGATGCCCTGTG
ATCTTAAGCCTCAGTATACAAAACACATGAAGAGACTTGCATTTT
TTTCCTCTCTTATTCCTTGTAACTTTTTCTTCCCATGATCTCTCT
TGCA

In some embodiments, the intronic domain that promotes trans-splicing primarily in the muscle comprises or consists of sequences from the gene CCZ1. In some embodiments, the sequences from gene CCZ1 comprise or consist of:

(SEQ ID NO: 1147)
TTGCTATAATAAAGTGAGACAGGATGATATCCATTTGCTGTCTTT
GTAAACGTGTGTCAATGGAAATCGTCACAGAATAATGGACAGTTT
CTGCCAATTCTCTATTCTCTCTGGACTATACACAGAGAGACTAGA
ACAGTGAACCCCTAAGTCCCCATCACACAAGTGAATAAGATTCTG
GTCTTCGTTGCTTCTCCATGTTGTGTTGTCGGGTTTGCAAGAGCA
TTTTAAGTCATCACGTCAGTCACGAACACTTCCCTGTGCCTCTCT
GATGAGGACATCCGTGGTAACCTCCCCACTGGCGCTGTCACACCT
GACAAGGTCAACAGCACTCCCCTCGCATCATGGAATGGGCAGTCT
GTGTCCAGATGGCTCTAGTTGACCAAAAAGCAGCTTCTTACAGTT
GACTTCTCCCCATTGACTTTGAACAGATTGTTATTATACGAAAGT
CTCCTTTTTTCCACCATTTCATAACCTCTGGTTTGTTCTGTCTTC
CCAG

In some embodiments, the intronic domain that promotes trans-splicing primarily in the muscle comprises or consists of sequences from the gene CCZ1. In some embodiments, the sequences from gene CCZ1 comprise or consist of:

(SEQ ID NO: 1148)
TGGGTACGCGCCTTTGTCTGATTTCAGCCTAAGCTTTTTGTACTT
TGCTGCATCCTATTCTTAAGTGATAAATATGTTTTGGGGGTTTCG
GTTGTTTGGGCTTTTTTTGTTGGAGACGGGGTTTCACTCTTGGAG
TGCAGTGGCATGATCATAGCTCACTGTAGCCTCGACCTCCTGGGT
TCAAGTGATCTTCCCACCTTGGCCTCCTAAGTCGCTGGGACTGCC
AGTACACACCGCCGGACCCAGCTAATGTTTTTGTGGTTGTTTGTT
TAGACAGGGTCTCACTATGTTGCCCGGGCTAGTGTCAAACTCCTG
GGCTCAAGCGATCCACCCATCTTAGCCTCCCAAAGTGCTGGGATT
TCGGGCGTGAGCCCCCACACCCAGCCCATTAAATGATAAATGTGT
TGTCATAAGCAATATAGAGGCATAGAGAGGAGCCCTGTTCCCCAT
CTGGGACCCCCCTGTCTCCTTAACTAGCCAGTGCTCATATTTTTT
AAGT

In some embodiments, the intronic domain that promotes trans-splicing primarily in the muscle comprises or consists of sequences from the gene GTF2I. In some embodiments, the sequences from gene GTF2I comprise or consist of:

(SEQ ID NO: 1149)
TATGATAAAAAATAAATAAATTAATTAATTTAAAAAAAAAGACCC
CAAATGTTTGCTTTTAACAAAACTGAATTAAGAGAATCACTGCAG
GCCGGGCGTGGTGGCTCACGCCTGTAATCCCAGCACTTTGGGAGG
CCGAGGCGGGCAGATCACGAGGTCAGGAGATCAAGACCTTCCTGG
CTAACACCGTGAAACCCCATCTCTACTAAAAATACAAAAAGAAAT
TAGCTGGGCATGGTGGCGGGTGCCTGTAGTCCCAGCTACTCAGGA
GGCTGAGGCAGGAGAATGGCGTGAACCTGGGAGGCGAAGCTTGCA
GTGAGCCGAGATCGCGCCACTGCGCTCCAGCCTGGGCGACAGAGC
AAGACTCTGTCTCAAAAAAATAAAACAAAACAAACAAAAAAGAAT
CACTGCAAACAAGAGTACTTTCTAGCAAAATCCATTCTGATTTGC
AACAGCACTGATAAATAACATGGTTATTGGGTTTCTTTTTGTTTT
CCAG

In some embodiments, the intronic domain that promotes trans-splicing primarily in the muscle comprises or consists of sequences from the gene GTF2I. In some embodiments, the sequences from gene GTF2I comprise or consist of:

(SEQ ID NO: 1150)
TAAGTAAAAGCCAGTATTTATGTCTTTAATAACATATCAACAAAG
GGCCATGTCTGAATGAAGTATAGAAGTTCGGGACCAGCCGGGTGC
AGTGGCTCACGCCTGTAATCGCAGCACTTTGGGAGGCCAAGGCGG
GCGGATCAGGAGGTCAGGAGATCGAGACCATCCTGGCTAACACGA
TGAAACCCCATCTCTACTAAAAATACAGGAAAATTAGCCGGGCGT
GGTGGCGGGCGCCTGTAGTCCCAGCTACTCGGGAGGCTGAGGCAG
GGGAATGGCTTGAACCCCAGAGGCGGAGCTTGCAGTGAGCCAAAA
TCGCACCACTGCACTCCAGCCTGGGCGACAGAGTGAGACTCTGTC
TCAAAAAAAAAAAAAAAAGAAGGAAAAAAAGTTCAGGACCTAAAG
AAGGAAGGTCCCAGAAACTGGGTTTCTGTTTCTTTCTACCACTAC
ACTTGCTACTGAAACCAAGCAGATGACTTCATTTCTCTTGGATTC
CACT

In some embodiments, the intronic domain that promotes trans-splicing primarily in the muscle comprises or consists of sequences from the gene POLR2J3. In some embodiments, the sequences from gene POLR2J3 comprise or consist of:

(SEQ ID NO: 1151)
GTGAGTCAATTAAACCTCTTTTCTTCATAAATTACCCAGTTTCTC
ATAGTTCTTTATAGCAGTGTGAAAACAGACTAATGGACCCTTCTG
GTTGAAGGAATGCAGCCATTCTGCTTGTTTGACTATGTCCTTTCT
ATTCATCTCTATTTCCTGGGAGGTGTTTATCCAAGTGCAATAGGA
GGTATTGGTGACCGCACAGTCCCCTCAGTGTTCTGCTAGTAAATA
GTTGAAGGTTGATCATTGATCTTCTGCGTTTTCAGTCTGGCATGG
AAAAGCCCCTGTGCAACTGGTAAAGATATCAATAAGCACCAGGAG
GTATCTAAATCCACCAGGAGCCATAGGCATCACGTTGACATCCAT
TTACCAGTCTTCCCTGGCAAGATTCTTCTGAATTGTGCTGCCTTG
GCCAAAAGAGGTATGGGAGGGGCTGGGCGCAGTGGCTTGTGCCTG
TAATCCCAACATTTTGGGAAACCAATTCAGGTGGATCGTTAGAGG
TCAG

In some embodiments, the intronic domain that promotes trans-splicing primarily in the muscle comprises or consists of sequences from the gene POLR2J3. In some embodiments, the sequences from gene POLR2J3 comprise or consist of:

(SEQ ID NO: 1152)
GAAAAGCCGAGGCAGGGTGATCACGAGGTCAGGAGTTCAAGACCA
GCCTGGCAAACATGGTGAAGCCCTGTTTCTACTAAAAATACAAAA
ATTAGCTAGGCATGGTGGCATGTGCTGTAGTCCCAGCTACTCGGG
AGGCTGAGGCAGGAGAATCGCTTGAACCCGGGAGGCGGAGGTTGC
AGTGAGCCGAGATTGTGCCACTGCACTCCAGCCTAGGTAACAGAG
CAGGACTCCATCTCAGTCAATCAATCAATCAATCAATCTCAGCGG
TTGAACTACCCTTGACATGGTTCAGCTCTGTATCCACACCCAAAT
GTCATGTCAAATTGTAATTCCCAGTGTTGTGGGAGGGACCTGGTG
GGAGGTGATTGGCTCATGGGGGCCGACTTCCCCCTTGCTGTTCTC
GTGATATTGAGTGAGCGCTTGTGGGATCTGGTTGTTTAAAAGCGT
GCAGCCCTCCCACTTCACTCTCTCTGTCTCTCCTGCTCCAACATG
GCCA

In some embodiments, the intronic domain that promotes trans-splicing primarily in the muscle comprises or consists of sequences from the gene ATP6V1F. In some embodiments, the sequences from gene ATP6V1F comprise or consist of:

(SEQ ID NO: 1153)
ACAGGGTCTCACTCCTGTCACCCAGGCTGGATTGCAGTGGCGTGA
TCGCAGCTCACTGCGGCCTCGACTTCCCAGGCTCAAGTGATTCTC
CCACCTCAGCTTCCTAAATAGCTAGGACTACAGCTTCCCAAGTAG
CTGGCACATGCCGCCACATCCAGCCATTTTTTTTTTGTGTGTATA
TATATAGATAGATAGATAGATCCGCCACATCCAGCCATTTTTTTG
TGTGTGTATATATAGATAGAGAGAGAGAGAGAGAGAGAGGGAGAT
GGGGGTCTCACTATGTGGCCCATTCTGGTCTCAAACTCCTGGGTT
CAAGCAATCCACCTGCCTCGGCCTCCCAAAGTGCTGGGATTACAG
GCGTGAGCCATCACAGCTGGCCTTCAGTATCTGCACATAAAAAGG
GATCTGCATACATATAATCTTATCCTTCCCCTTTCTGACACATCA
CTGCATATTGAATACACCAGTGTGCACCCTAATCAATCTTCATTA
CCAG

In some embodiments, the intronic domain that promotes trans-splicing primarily in the muscle comprises or consists of sequences from the gene ATP6V1F. In some embodiments, the sequences from gene ATP6V1F comprise or consist of:

(SEQ ID NO: 1154)
TAGGGTTGACAGAGGGTTGAGCGTGGCAGCCTTTCCCCTTGTCCT
CTGTGCCCTCTGGGTCATGCTCATTCCCCTCCACAGCCCAGCCCA
ACAACTCGGAGAGGGCTGCCTGGGTAGGAGAGACGGCAGCCCCCA
GAGCTGTCCTGGACTCCCTTCTCATCTCTCCCCACAGGCAATTTC
TAAACCGGGATGACATTGGCATCATCCTCATCAACCAGTACATCG
CAGAGATGGTGCGGCATGCCCTGGACGCCCACCAGCAGTCCATCC
CCGCTGTCCTGGAGATCCCCTCCAAGGAGCACCCATATGACGCCG
CCAAGGACTCCATCCTGCGCAGGGCCAGGGGCATGTTCACTGCCG
AAGACCTGCGCTAGGGGACTCCTCATAGCCCTCAGCCCTTCCCTC
GTTTCCAGGCCTCTCCCCAGGCTTGCCATCAGCCTTCTTTACTTT
TTGAGCCTCTGATTTCCAATTCCCTGCTCCTTCCCACTCCATTAA
GAGG

In some embodiments, the intronic domain that promotes trans-splicing primarily in the muscle comprises or consists of sequences from the gene ASAH1. In some embodiments, the sequences from gene ASAH1 comprise or consist of:

(SEQ ID NO: 1155)
GTGGATACTCTTTTATTCTTATGGGGAAATGGAGGCGCACAAGTT
AAATACTGAAATGTTTTAGAAAACTGTTTAACGAGAGGTAAAATG
TGCAATTATGGCTA
AGCTTTAGTCATTAAAATAATTTGGTTTATTAGATTAACCAGTTC
TCTCAAATTCTAATTAGTGCAGGTTTGATGGTAGAGAGGTTTGTG
GGGTCTCATCATCCCCACTTCATTGTAATCCCTTCATTGTCCGTT
GTGTTGTGTTCATCCCCTCTCATCGCGTCTCTGAGATCCATGAAC
CTACCCAGGACTCTAGGCTGAAAAAGAATCAGGAATTTATGGCGG
GTATAGAGGCATGATTTGGGATTGGTTGGATGTTATGAGGTGAGG
AGCAGCTGATTGGAGTTCATAGGGATGAGATGCATATCTCCAACA
GGCCACTGAGTTATCTTTGCTACTAGACATAGAAATCTAAATGCA
CAGTTGAAGGGAGAGGGGAGACTAGTAATACCTGC

In some embodiments, the intronic domain that promotes trans-splicing primarily in the muscle comprises or consists of sequences from the gene ASAH1. In some embodiments, the sequences from gene ASAH1 comprise or consist of:

(SEQ ID NO: 1156)
ACTGTGGTATTTATGTACCTTTGAATGACAAACAGACGTTCTGTT
CCTTTGAGATCATCCTTTTCAATTAAGTGCGTAGATTTGGGCAGC
ATCCATGGGCAGATATGAGGGAAGAAAAAGATAACCTGTTTGGCC
AGCATGACTAGTCAAATCAATGCTGAAGATAAAGGAACTGATAGC
CAGATCACCCCTGAGTCCATCTGAGTCCCATTCAGCATTTCTTTC
CCACTGCTCACGGGATGCCTAGCACTTGCTCTCTTATTTGTATAT
GTGTGTGTTGTGTCACTTGGACTAGTTTCCATGCCACCTAGGTAG
CCATGTGGGTTCAGGTGATGTTGCTGATATTATGAATGGCATGGA
CATTGCAAACACTAAGTCTCAAAAATGATGAAATGATATTTCTTG
TTTGCAGTGAAGTAGGAAAATAAATTGTGATGACTACTGTTGATT
GAATACCAATGCTGCTAAGTGTTTTTTTTATTTTTGCATTTTTCA
TATA

In some embodiments, the intronic domain that promotes trans-splicing primarily in the muscle comprises or consists of sequences from the gene CA3. In some embodiments, the sequences from gene CA3 comprise or consist of:

(SEQ ID NO: 1157)
GGCAAGATAAATTAAGTATTGAGAATGCCTTCTAGACATCTTCCC
CAACCAAATGGGGAAAGTTGAGTATATTTCTTACAGATGCAGGCA
AGGCTTTCAGTTTGACCTTTGCACATGGGATTATTTCATACTTTT
GGTCTTGAATAAAACATTTTTTAACTGCTGCAAATGATTGTATCC
TCTTGCCATTCTCTGTAACTCAGCATAGAAAGTTTGATTAACCTA
ACTGGTGCTCATGTTCTAAATTAAGGTCAGGTTGCCACCAGGTTT
CAGAGCTCTGGTTGATAAAAGGAGATAAATAAATAATTTTATATG
CTTTTTATTTGAGCCACTAGAAATGTTACCAAAATGTTACATGCT
ACTCTTTCAAATTTTCTCAGCTTTTATGAGAGTGCTCAGCAACCT
TAGCCTGTACAATGATTGGGTTTATTGTGGATAATGTTGTACTTA
TTTCCATATTCCAGGAATTTACCTTCTAATCTGTCTTCTATGGTT
TCAG

In some embodiments, the intronic domain that promotes trans-splicing primarily in the muscle comprises or consists of sequences from the gene CA3. In some embodiments, the sequences from gene CA3 comprise or consist of:

(SEQ ID NO: 1158)
TAAAGTAAAAATTGACTATATATTTTCTTCCAAAATGTATTTCCC
TGCCAATGGTTAGCATAGTTTGAGTTTCATTTCCTCATTTTTACC
TTCCTCCTTTCATATGCATTTTATTATTCATTCAACTAATCAAGA
TATTTCTGCAAAGATCAGCTCAAAATGTCTTACTTTTCTCTAAAA
GTGTATCTTTTACCACACTCCAGGACCCCTAGCACTTCTAAATTT
TCCCCAGATGTGGGAGGTGTGTGTGTGTGTGTAGGTGTTGGGGAC
AGAGCCTTAGAGTGAGCTTATTACGGTGGTGGTAAGGGCAAAGGT
GGCATATATTTATTTTCCTTTCTCACTGCTCAACTTCAATTGCAC
TGGAAACCAGAAAAGTCAGTGTTGGGAACCATATGATTCCAAGTC
CCTGGCTGGACATCCCCTCGGCTTGAGGTGGTCACCACTCACGTG
ATCCCAGGCTAAGATAGCACATGGAATGTGTGTGTTGTTGAGCCA
ACCT

In some embodiments, the intronic domain that promotes trans-splicing primarily in the muscle comprises or consists of sequences from the gene YWHAZ. In some embodiments, the sequences from gene YWHAZ comprise or consist of:

(SEQ ID NO: 1159)
GTAGACCGGGGCGAGGCTGCGCGCCGGCGGGAGGGGAGTCCACTG
CGGGGTGAGGTTTTGGGGTGGGGCTCGCCTGGGACCTAGAGCTGA
ATTTCCTGGGCTCCGCGGTGGGAGGAAGGACTTGAATTTTCCTGG
TGGGGGTGGGGGATGGGTCTGACTTTTTTTGCTCCTTGTGAACGG
GCGGGGGAGGTGTGGACGGGGGCTGCAAGTCCAGGTCGGGGTAGA
GAACAGGGTGGTGAGCCCGCAGTCGGCTCCCGTGCTTGATGGACT
AGAACTGGAGGGTCCCTGCGCTCGGTTTGGGGACGCATATCCTGC
TTTGGGGGCGGGTGAGCCTGAGCATAGCCGCTGGTGGCCCGCTCG
GCTGGGTGTGGCTCTCGTCCCCTCCCCCACCTGCAGCTGTCGGTC
CCTTAGAGACGCTGTACACGGTCACCGGCGCTGATCGGTGGCGGG
GAAGGCGCTGGGTGGGAGAGCGGGGTGTGTCCGCGGCGGGGCTGG
TAGT

In some embodiments, the intronic domain that promotes trans-splicing primarily in the muscle comprises or consists of sequences from the gene YWHAZ. In some embodiments, the sequences from gene YWHAZ comprise or consist of:

(SEQ ID NO: 1160)
CGCCCCGCGGTGGCGGCGCCGGGCTGAGGCGGAGTCTCCTCAGCG
GACACGGGCGGCGGCTCCTCTTCCGCTGGGCTCGGGAGGGCGGCT
AGCGGCCGCCTCCGGCTCCTTGCTTCCGCATCGACGCCCCGGCGC
CGATCGGTGCTCGCTCCCCCGTCCTGCCCCCGGGGGTGGGTAGGG
CGCCCACACGCGAGATCCCTGGGGATCTCCCTGTCGCCGGCTCCC
GAAGGTGGACGAGGCATGGCCGGGGCGCCGCCGGCCGCGATCCAT
CCCCACCCTTGTCTCTGCGGGCTTCCACCCCCTCCCTCGCCGCCC
TTCTCGCTTCCGCCCGAGATAATGACTCGGCGGCGGCGGCGGCGG
GCATCGCAGTGGCGCAGGTGAGGCCGCCCCCTCCCCTCTCCCTCG
GCCGCCCCCTCCTTTCCCTCCCCCTCGCGCAGCCTCCGCGCTGGG
CGCTGCGGCGGCGGGACCCGGCCCTCCCCCACCCCGGGAGGCGGT
GGCG

In some embodiments, the intronic domain that promotes trans-splicing primarily in the muscle comprises or consists of sequences from the gene SMARCA2. In some embodiments, the sequences from gene SMARCA2 comprise or consist of:

(SEQ ID NO: 1161)
TAATTGAAGAGGGAGCTAGTTTGCATCTCGTTCTTGCACACCCTG
AGTCATGAAAAACATTTTCTTTTAAGCAAAATTGAAAGAAAGAAA
AGAGAGAAAGAGGGCCAAAAAAAAAAAAAAAAAAAAAAAAGGTTG
CCATCCAGTGGAGGAAGGAGTAATTCCCAGCTTCATTCAGGAAAA
ACGCAGGCTGAAATATGCTTAACTGTTTTAACTTGACTGGGGATT
TTTACCACCATATGGGTTTTTTTTCGTGACATCCGGAGCCAAACG
GGTGCAGTGTGCCTTTAAGAAAAGAGGTGCCTCACTAAACTTCGC
TGTAGTTGTGTTTCACCGTCTCGGTAGGGAAGGAAAGTGTTAATT
TCTTATTTGTACACTTTTTTTCCCCGACTTCCTTCCTATTCACTT
CATTAAATCTAGAGGCAGTTGAGCATGGGAGCCGTCTGTATGTTG
AATTAGGGCTCGCACTCTTGCGCAACACGTCACCAGTCGGAAACT
GGGG

In some embodiments, the intronic domain that promotes trans-splicing primarily in the muscle comprises or consists of sequences from the gene SMARCA2. In some embodiments, the sequences from gene SMARCA2 comprise or consist of:

(SEQ ID NO: 1162)
TCAGTACTTTGTGTGTTTCCTTGTAATTCCAAAACCTAAATTTAA
TAAGAATCTGCACGTATTAGCAGTTGTAATAAAAATTGTTTCAGT
TGTTCTGTTTGCAATTTAATTTGTTTTCGCTTCTTAGCTACTCAC
AGATTTAGAGATTTAAAAGTCCAAATATCACAAGCTTATGCTTTT
TCCCTCTTTTCAAAATTAAGGCGGGGTGAGAGTAGAAATATCCTT
TTTACAACTCCCCCCAACTTCATTTATCTCAGTGAGAAGAAAAAC
AATGAAAAATTTTACAGCATGAAACTGAAAGTGAAGTGTTACAGT
TGTTCTGAATTTTCTTTAAAGAGCAGAATAACATATTATGTTAAA
ATATTTGAATCTTAGAACATGTACATTGCTTTTATTTACATGCAT
TTAATTTTCTTAAAAAATTGTCTTCTGTTTTAAAAATCATTTAGA
CTGCTCCAAACCATGCTGCTATTTTTTAAAAAATAAGGTACAAAA
TACC

In some embodiments, the intronic domain that promotes trans-splicing primarily in the muscle comprises or consists of sequences from the gene PLIN2. In some embodiments, the sequences from gene PLIN2 comprise or consist of:

(SEQ ID NO: 1163)
AGTGTGGTGACTCGGGTGGTCAACCTGCCCTTGGTGAGCTCCACG
TATGACCTCATGTCCTCAGCCTATCTCAGTACAAAGGACCAGTAT
CCCTACCTGAAGTCTGTGTGTGAGATGGCAGAGAACGGTGTGAAG
ACCATCACCTCCGTGGCCATGACCAGTGCTCTGCCCATCATCCAG
AAGCTAGAGCCGCAAAGTGAGTTCTGATTTTTCAAGATGCAAGTC
AAGGGACTGGTCAGTGGCGAGCCCCTGAGCTCACAGCAGTAACAT
CTGGAACTCCTCAGCCCTTCCTGGGGCATACACTGCCTTTACCTA
GAAACTTTCAGGTGACCCTCATTACCGTAAGAGGACTGTTTCTTT
TCTTTTCTTTCCTTTTTTTTTTTTTTTGAGATGGAGTCTCACTCT
ATCACTCAGGCTGGAGTTCAGTGGCGCAGTCTTGGCTCACTGCAA
CCTCTGCCTGCTGGGTTCAAGTGATTCTCCTGCCTCAGCCTCCCG
AGTA

In some embodiments, the intronic domain that promotes trans-splicing primarily in the muscle comprises or consists of sequences from the gene PLIN2. In some embodiments, the sequences from gene PLIN2 comprise or consist of:

(SEQ ID NO: 1164)
CTAATTTTTGTATTTTTAGAAGAGACGGGGTTTCACCACGTTGGT
CAGGCTGGTCTTGAACTCCTGACATCAAGCAGTCCACCCCCCTCG
GCCTCCCAAAGTGCTGGGATTACAGGCGTGAGCCACCGCGTCCGG
CTTGGCTAGCATTTTTGTTAACAGATACTCGGAGAAACAAGAGAA
GTATGTCAACAACTTTACTTCTCCCTCCTGAGTATTGCAAGTTTA
CACATAAGCTCTGAAACTTTGCCTCAAAAGAGGGAGTGGGGTTGT
GTGGGTGTGGGTCTTTCAGGAGGGGGACCTGGAGAGCTGGAGAGA
GGAAAGAAGTGCAGAAGACTACAGGGAGCTCACCCTGCATTTTTC
TCTACTTGTAATCAGCATTTGCTTGGGTTGGGGAATGAATTTGGG
AGAGTCTTATCCCGGTGTTCTGAAATAAGTCTCTTTTCTGCAGAT
TTCTTTCTCCAGGAAGAAAAATGGCATCCGTTGCAGTTGATCCAC
AACC

In some embodiments, the intronic domain that promotes trans-splicing primarily in the muscle comprises or consists of sequences from the gene SURF4. In some embodiments, the sequences from gene SURF4 comprise or consist of:

(SEQ ID NO: 1165)
GTGGCATTCTGAGCAACAGGGAAGCCTTTGAAACAGGCGGTTGGG
TAACTGTTGAGAGCATTCGGGAACTGGGTGGACAGGATCCGATCC
TACAAAGTGAAGGGACACAACTCCCTTCCCTCTTTCTTGATTAAG
CTTGACAGAACAAAAAAACAAAAACAAAAGGTGGTGGTGGTTACG
CAGATTTATGCCTTTTACTTCTAAAAGAATCCCGTTATGTCGACA
AATTAACAGCCTTATCCCCGCAGGGAGAGCCTTGTGTGGCCCACT
TGCCGTTTTTTCCTCGGTTTACTTAATGTGTTCGTGTGGCTTTGC
TAGAGTTTTAAGTAGTGATGGTTAAATTTTTAAATTTTTCTGGAG
GTGGTAGTGAGTCATCAAACTCTGAGGCCTAGACTTATCGGAAGG
CTTGTATATGTGTGGTTTAAAAAAAAAAGAAAAGAGTAAGTGACC
GTTTTCTGATCAGCTATTGAGCTCTTCCGTTCCTCCTGGGGGGTG
ATCT

In some embodiments, the intronic domain that promotes trans-splicing primarily in the muscle comprises or consists of sequences from the gene SURF4. In some embodiments, the sequences from gene SURF4 comprise or consist of:

(SEQ ID NO: 1166)
CCCGACCCAGGCCGCCCGGGCCGCTCCGGAGTCGGCCTCCCAGCG
CAGGGCGCCAGGCCCGGGCCGCCCCCTGACTCCTTGTTCCCACAG
AGGGACCCCCCCGCCCGGGCCCTCTGTCCCGGCTGGACAGCCCCA
AACCCGGCCTCCGGGCCCAGTCTCCCCTGCCCGCTGGGCCGCTCC
CGGAGCCCGGGCCTCGGCGCCCTGCCTGGCTAGCCGCCCCCACCC
TCCAGCCAGCCCGGGGCGCGGGCTGTGGGGGTTTCTTCCCGGGCC
GGCGGCGCGAGTCCTGGCGTTGGGTCGGGTCTGAGGCTCGCGGGG
AGCGCCTCGGCGTTTGGAGCTGCTGGGGGGTCCCCGCACGTTTTT
CCTCTCACTGGGGCTCCCGGAGGCGGGCTGGGAGCTCGGGGACGA
ACGGGGGCCGGGGGTCCTCCCAGAGGAAAGGGGGCGCGCTGGCGT
GCGCCCCAGCTACAGGAAGCCACAGCACCTTTTCCTTTTCTCTTT
CCCA

In some embodiments, the intronic domain that promotes trans-splicing primarily in the muscle comprises or consists of sequences from the gene DNASE1L1. In some embodiments, the sequences from gene DNASE1L1 comprise or consist of:

(SEQ ID NO: 1167)
CCCTATTCAGGCAGGAGCTGCTCTTCTGGGGTATCGCGATCCACT
TAAGGATGAGGCAGACTTGGTGACAAGCTGGTCTGAGCAGGTATG
GGAGCCCCCTGGGGAGACGGAAGAAGGGAGGAAGTTGCCTTCTGC
CTGGGGAGGGTTTGAGAGGGAGAGGGAAGCCTAGGGCTCCCACCA
AGGCTGATATTGACAGCCAGGGTTTGGGGCTGAAGCCAGGAACCG
TCACTCTCTCTGGTTCTTACTGGTAGCCCTCATGGGGGTCCCTGA
CGCCAGAGCCTCCAAGGCTGCATGTGCCAGCCCAGGGCTGCCCAC
ATACCCATGTATATCCCAGAATAGGCACCAGGGTAGGGAACCCAA
ACTAGCATGAGTGACAGAGCAGGTGGTCAGGGAGAAACAGACATC
AAACCCAGCCAGGAGAGAGACACCGCAACAGAGAGACAGAGAAGG
GAAACCAGAGACGAGAGGGAAAGTGAGACAGAGACGGACAGAGCC
TCAG

In some embodiments, the intronic domain that promotes trans-splicing primarily in the muscle comprises or consists of sequences from the gene DNASE1L1. In some embodiments, the sequences from gene DNASE1L1 comprise or consist of:

(SEQ ID NO: 1168)
CCTCCCTTGACTTCACGACTGTGGCCAGATCATGTGTGGACTGTC
CCTCTCTTTGGGTCTCCAGAGCGCTTGCATCAAACACCCCTAACT
CAGAAGTGTGCAGCCACACTGGGACTCAGAACCCAACAACAGGGA
CAGAAGACTCACGCCCTTGGGGTGCCCGGTCTCGTGGCATCAGGC
ATGACTTCCAGCTCCTGCGCCTTCCCCAGCAACTGCTGACTGGGG
ACCCAGACCGGGAGCTGAGCGACGGGCCTGGCGAGCGAAGCTCGG
GGTCTCACTCAGGCACCAGCCCCTCCTTGCCCCAGGCTTGAGTGA
CTCACAGGTCTGTCATGTCCCGCTGGCCCGGGAAGGCGGGGTCTG
GGTGGGAAGGTGGGCGGGCAGGTCAGCAGTTCCCAGGCCTGGTGC
TGGTCCTCCTTGTTGGCGGCGAGGCGGAGACGGGGCGGGCCTGCC
TGTTGGCAAGGGTGGGGCGTCCGGCCCAGCCAGGCCCTTCCTGGC
CGGG

In some embodiments, the intronic domain that promotes trans-splicing primarily in the muscle comprises or consists of sequences from the gene IDI2. In some embodiments, the sequences from gene IDI2 comprise or consist of:

(SEQ ID NO: 1169)
GTACACACAGCATCCAGTCTCAGCTGTGTCCACGGGAGTCCAGGT
TGGCAGCGGGCAGACGGTGAACAAAGTCCAGGCTGGATCTACACC
ATTGACCTCATGAAGCACCATGAGGATGAAGGATGGGGCTGTGGA
TGGGGCTGTGAATGAGCTGTGAATGGGGCTGTGAATGGGGCTGTG
AATGTAGCTGTGGATGGGGCAGTGTGAGTCCCTATGTGTCCCAGG
CAGGGTTGAGACTGTGCAGGAATCTGTCGACCACACACCGAAGAG
GAAATTCAAGCCGGGCGTGGTGGCTCACGCCTGTAATCCCATCAC
TTTGGGAGGCCGAGGCGGGCGGATCACAAGGTCAAGAAATCAAGA
CCATCCTGGCCAACATGGTGAAACCCCGTCTCTATTAAAAGTACA
AAAATTAGCTGGGCGTAGTGGCACACGCCTGTAATCCCAGCTACT
TGGGAGTCAGAGGTTGCAGTGAGCCGGGATTGTGCCACTGCACTC
CAGC

In some embodiments, the intronic domain that promotes trans-splicing primarily in the muscle comprises or consists of sequences from the gene IDI2. In some embodiments, the sequences from gene IDI2 comprise or consist of:

(SEQ ID NO: 1170)
TCTCAGTAAGGAAACTTGGTCAAAGGTATTCCAGCAGTCTTCCTG
TGAAACCATGCTCACTGATCACCACCCTCAAAGTGCAGCCCAGCT
TCCTCAGCCCAGCTGGTGCAGCCACCCAGCCCACAGCCCCACCAC
CTCCTCCCGCCACCCCATGGCATCTCAGGTCCTCTGTGGGTACAA
AACTGAGTAGGAACCAGTCCCTCTCCTAAGACATACTGGGATAGT
GCCACGGGACATGTGTGTGCACATCTGCAGGAAGGGCCGCAGGAG
CGGTCAGGGCCAGCCCCGCCAGGTGGTCATGTGGAAGACCCTCAC
CAAGCCCACAGCCGTGGAGGCTGGAGGCGAGTGCTGCTCTGAGAG
CCCATGACAAACCCACTCCCCCCGCCAGGCTGTCTTCTGCTGATG
ACGGATGATGGTTTTACATGAAGCATCTGCATTTGTATTTAATAT
ATTCACTTTTCAGGAATAAAGAGGGATGTTCCCACATGCATTTTT
TCCA

In some embodiments, the intronic domain that promotes trans-splicing primarily in the muscle comprises or consists of sequences from the gene AKR1C3. In some embodiments, the sequences from gene AKR1C3 comprise or consist of:

(SEQ ID NO: 1171)
ATGTTGCTTTAGTTTCTAAGCAACATAAATAGCTATTCTTAAGCA
TTGGGTTGAATGGATAGAAGAATTAGACTGTTAAAATGAGTTGTA
AACTCTACTGAAGATAATTCAGGTAACATCATAGTTATTACTTAA
TACTAATCTTTACATTTTAAGAATTTACTCCTATCATTCAGTAGA
TGTACAAACTATACATCCAACGTATAATAAAGTTTATAAGGATAG
GTCAGAGCCAAAGGAATCATGAGAAGGAAGAGAATATGTTTGCCA
GTGGTCATAAATTTTGAAGCAGTAGGAAAATATCTAAATACTAGA
TGGCACAAAGTAATAAGATTTGCTCAAGCATTCATTCAAAATCAC
CTCCATTCTTTAACCTCTGCAGCTTTGGTCCACTTTTCATCGACC
AGAGTTGGTCCGACCAGCCTTGGAAAACTCACTGAAGAAAGCTCA
ATTGGACTATGTTGACCTCTATCTTATTCATTCTCCAATGTCTCT
AAAG

In some embodiments, the intronic domain that promotes trans-splicing primarily in the muscle comprises or consists of sequences from the gene AKR1C3. In some embodiments, the sequences from gene AKR1C3 comprise or consist of:

(SEQ ID NO: 1172)
AAGACATGGCTACATTTAAGGATATGAAATAGTCTACAAAAATGT
ATCATTTGTCTATTCTTATCTCTAAACTCCAACACTTTTTTTTAT
TATTGTTGACCCAAACTTTTTCATTGGTGGCGATTATGGCTTTGG
AGCAGCTCAAGGCTCATGGTCATCACCATAAGACAACTCCTCTGG
GTCTCCCAACTTCTGCTGACCCTATGTGATTCACAGTCCTGCCCA
AAGACTATCAACTCTTCAGGTGGTCTGATAATCCTACCGTGTACA
AGAGTACAATCCATTATTTTTGGATATCAAGGTATTTCCAAATCA
AAGAGAGGTACTTCAATAGAGGAAATTATAAGGGCGGAATAAAGT
GACATAAGTCAACTGTGAAACTTGATTAAGACCTAGAAAATGGTC
CTTTTCCATGCATATAATATTTGTAAGAGATTAGAGGAAGCCTGT
CTCCTGAATACATTCCTTATACCTTCATATGTAAAACACTTAGCA
CATA

In some embodiments, the intronic domain that promotes trans-splicing primarily in the muscle comprises or consists of sequences from the gene TIMM23. In some embodiments, the sequences from gene TIMM23 comprise or consist of:

(SEQ ID NO: 1173)
GTAAGTCTCTTGTAACCATCTGATGTAGTGATACTTGAATATTAA
GCTCTGTTGTATTGGTTTGATGAGTACAACCTTGAGATTACAGAT
GGTTAGCATAGTTATGTGCTTTTTTGTCTTTGTTTTTTAATCTTA
ATCAAAATAAAAGCAACTAAGGAATCAGATTACTGACTCTAAGCT
TTTAAAAAGGAGTGATTTTTTTAATCAGTGTCCCTCAGTCAGCTA
GTGCTATGCCAGCCTGACTTCTCAGCTGCACCTGTTACATTCTCC
AACCCTATGTGACTTGCTTTTCAAATTGTTGAGTTGCTAATGGGA
ATTTTGGATTGTTTGGGGGCATGCAGATCCATAGGAAATGGTAAT
GAAGAATAAGCAGCTCATGAGAGTGATGGCTCAAGCTGCCATCAT
TTAACTTGAACTTCTTACTGTCTTGACTTTATGTTTCCGAAATGA
TGAGCCAGATACATTTTAAATAAAATTTTTAGAATTAGAGTTAAT
TCCA

In some embodiments, the intronic domain that promotes trans-splicing primarily in the muscle comprises or consists of sequences from the gene TIMM23. In some embodiments, the sequences from gene TIMM23 comprise or consist of:

(SEQ ID NO: 1174)
TTTCACTTTGATTTAGGTGTTACTGCTGCAGAAAGTTTTATTTTT
GTTTAAGTATAGGTTTGAGTTAATTTAAGGTTTATTTTCTGTGAA
AAACTACTTTGAAATGTTAAAAAGGCAACTTTACAAGATTTGTGT
CTTGAGGGACACTCAGCTTGGTTTTCATTATTATCCTTTTAGGAT
ACAGATGAGTTTATTTTACCTACCGGAGCTAATAAAACCCGGGGC
AGATTTGAGCTGGCCTTCTTTACGATTGGAGGATGTTGCATGACA
GGTGAGTGTTACATACTTTTTTCTCAAGAGTGCTCAGTTCAAGTA
GTTGAGGGTGCGTAAAATCAAAAGCAATTAGAATGTTGGTTTCAG
GGTTTTTTTTTTAATATTTACATTTGTCTTTTTCTATTAAATAAA
AATGAAAAAGAATCAGAAGTGTAGTTATGCAGATTTGAGTTTGTT
TTTTTAATATAAAGCTGTCTTTATCTGGGTGAATTGTTATGATTT
ACCA

In some embodiments, the intronic domain that promotes trans-splicing primarily in the muscle comprises or consists of sequences from the gene MYPN. In some embodiments, the sequences from gene MYPN comprise or consist of:

(SEQ ID NO: 1175)
AGTAAAGGAAGCTTTGTTGTGTTTTAATGCAAACCACACAGTAAA
GCTTTAATTTAATCACCTTAAAATTAGGGGAACAAACAGGAAGTG
TTGTAATAATTGATTGTTTTTTCGTATTTACCTTAATAAATGCCT
TGACCATAGTCCCAGTTTGGTACCAGATAAAGATATTTATACATG
TAATATGCATCCTGTGTATAGATATCTTATGTATTCCATATGCTT
TATTCTTGAGAATTTCTTCACTCTTATTTTCATTTAGACTAAGGA
GAAATCAAGAAAGCCTATTTCCTTAGTCATCTTAGTCATTATCCA
CCCCCACCAGCCCTGCAGGCTAAGAACATCGTGCAGACTGAGTTA
TTAAAGTTGATCTGAAAGAGCAATCTGTTCTAAATGACTGTCCCT
GGATCTGCAACGGAACCCTGTAAATACCCTTCATTACTCTGGCGG
GGCAGTATCACATTTCCTTCATGGCACTTTTGTGTCTGACACAAG
TTAG

In some embodiments, the intronic domain that promotes trans-splicing primarily in the muscle comprises or consists of sequences from the gene MYPN. In some embodiments, the sequences from gene MYPN comprise or consist of:

(SEQ ID NO: 1176)
TAACCACAAATTTACTTCTCAGGATAATGTTTTAAAGTGGGTGTT
CTGTTTAGTTTTTAAAAATGTTTTTAGTTTTTAAAACTAAGTGGG
TTTTGAACTTTTAGTTTTTTTTTTTAATGTTTTTAGTTTTCCCAG
GCAAGCATTTTCAGTAGTTTAGATGAAAAAGAAATTTCTTAGTGA
TTTCATTGTCTTTTTCAATTATATTGTTGTCATGCCTTTTATATA
AGATCTTTTATAATCCACCACCCATCTATTGTTTTGGATAGTCAA
GAACTTGTCTTTGCTGTGAAAATTTTTCTCATTAAAAATGGCTAA
AAAATACATACTTTCTCTGTTTTTCAAAAATACGGCATAGAGGTT
TTGTTTAGTTTTTTCATCTGATACTGGATATGTCAAGATGAATCT
ATGAACTTCAAGCAAAACTTATTGAAGCTTTGGTATATGTGAGTA
ATTTGTTTTAGAGAACTGGAGCCTATAGCGATATAAAAAAATAGC
AAAT

In some embodiments, the intronic domain that promotes trans-splicing primarily in the muscle comprises or consists of sequences from the gene TNNT3. In some embodiments, the sequences from gene TNNT3 comprise or consist of:

(SEQ ID NO: 1177)
CCACCCTCCAGCACCGCCAGAGGCCCCAGGGGAGGGCGGCTGTGC
CCACGGGGCTTGTGCTCTACCCAGGTGCAGCAGGTGTGGGGCGAC
CAGTCCAAGCCAGCCGGGCAAATCCGAGCTCCTTCTCGGGGGTTC
TGGGCAGGCCACCTGTTCCACCTGAGCTTCCACGAGCTCCCCTCC
GACAGAGGCAGGGCGATTACACTCCCTCCCAGAGCTGTCCCCAGG
GGGTCCAGGCATGAGGGACAGCCCTGACCTCACCCTTGGGGCTCT
CCCAGAGGGGCTGGCCCTGAGACGGGCTTCACGGCACAGGACCCA
GGTCCTTCCCATGCTGTGTTCCCAGAGCTGAATCAGGGACCCACT
GGCTTTGGATAGATGCGGCCACAGCGGGCCCCCAGGGGCTCCAGA
GCCTGGAGGGCCATCACCAGGCCAAGCTAGCCCACAGCCGGGCCT
CGATGCCCCAGCCGCCCATCCAGCCTTGTAACCATCTTGGTCTTT
CTAG

In some embodiments, the intronic domain that promotes trans-splicing primarily in the muscle comprises or consists of sequences from the gene TNNT3. In some embodiments, the sequences from gene TNNT3 comprise or consist of:

(SEQ ID NO: 1178)
TAAGTAGCCGGGTCCGCCCTGGGCCAGGCCCGTGGGTGTGCGTTG
CACGGTGAGGTGAACCTCTCGCATGGCAAAAACCTGGATCGCTCA
GGATGCTGGCGGCAGGGGGCCTGGAGCCTTCCTCCTGCCCCCGCT
CTCCCCTCGTGCCTGCAGCCGGGGGCTTCCCAGTGGCCGCTGCCC
TGTTGGCAAGCGAGGAAGGGCCCCCACCTTCCCAGGAGCTCCGCC
TCAAGCCAGCAGCGGCCACAGAGTGCCCCTGGGCACAGGCACGAG
GGCGGGAGCGGCTGGCCCAGGAGCAGCAGGGCCGTTCGGAGCTCT
GAGCTCAGCGGCTGAAGGGAGCTGGAGCTGAGAAGGAGGGGTCCG
CAGGCCTCACCTGCAACCCCTCCCCAGCCCCATCCGTGCTCCGTA
CCCCCCGCCCCACATCCTGCCGCAGGCTGAAGGTAAGTGGCCAGC
TACGGGCTGCTGTGTGTGGCAGTCCAGGCCAGTACAGGCTGGCGA
GAGT

In some embodiments, the intronic domain that promotes trans-splicing primarily in the muscle comprises or consists of sequences from the gene ZBTB16. In some embodiments, the sequences from gene ZBTB16 comprise or consist of:

(SEQ ID NO: 1179)
GCTAGAAGGGGGAGCCCCGGCTGTCAGCCTGGGCGCAGCTGCCTC
CCCAGCCCTTCCTCTTCAGGGCACGGTCGGGGTGAGAGGTGGGGG
GCGTAGCGGTGTGCGGGGGCTGAGGGCGTGAGCAGAGGGGTCGGG
GATCCGGAGGCTTTGTACCGCCAGGGGCTGGCGGAGCAACAGAGC
CCGTGGGTGCTCTTATGTATGCGGACCGGTGCGCGGGCGCAAGAT
AAGGTTGTGGTTTATTTATTTGTGTGTTTATTCGCCGGCCGGCTG
GGAAGCTAGAATCGGAGGAGCTGACGAGTAGATCTGGGGGCGGAG
GGGAGCAGGACTGGGACTGCTTACGTTTTGTTTCTCTTTGAGAAA
ACGTGGTGGGCTTTTTCTTGATTGGACTTGATCCCCACCCCCCTT
TTGCAGGGGAGGGAGGGAAGCTCCAGAGGGTCTGCAGCGCTGCGG
GCCCTCCTCGGCTCTCGGCGGGACCGGCGGTGACACCGGAGCTCG
CCGT

In some embodiments, the intronic domain that promotes trans-splicing primarily in the muscle comprises or consists of sequences from the gene ZBTB16. In some embodiments, the sequences from gene ZBTB16 comprise or consist of:

(SEQ ID NO: 1180)
TGGGTGCCGGTCTCTGCACCTGATGCGTTCGGGATGCCTTTCCCA
CCCTGGCGCGCCCGCCGCTAGCTCGCACAGCGCCTCGCACACTCC
CGCACGCGCTTGAAATGCGCACGGTCCCGCCGGCCCGCGGAACCA
CCCGGACGCACGGAGCGCTCCGCACCGACTCGCTCGCCGCCTCCC
CGAGACGCTCGCACCGTGCTTGGGCCGGGCGCGCTGGCCGCTGGC
GCCGCTGGCCAGAGGCCTGGGACCCAGCCGGTCGCTCCCAGGGGG
TCACGGCCCTGGGTCGGAGAGGGAGGGCGGGCAGACCCCTTCTCG
CCTTTCCTCCCACAACTCGCTGCGGGGCTTTTGTGCTTCCCCTTC
GCCGCGGGGCGGGTCCGCCTCCCCTGCCGCTCTCGCCGCGGAGTC
CAGCCCGCCCGGACTGTCGCCGTTCCTCCCCGTCTCTTTCGCTTT
CCCTCGTCCCTAGCTCAGCTCTCCTTCTTTCAGGAGTCTAGCTCC
TCGG

In some embodiments, the intronic domain that promotes trans-splicing primarily in the muscle comprises or consists of sequences from the gene STAC3. In some embodiments, the sequences from gene STAC3 comprise or consist of:

(SEQ ID NO: 1181)
GTGAGTCTGGGCCTCTGACCACTGGGTGTGGGGGGCTGGGAAGCC
ATTACAGCTGAAGAAGAGTGCTTAAATTGATCTCATTTGTGTGTG
ACTCATTTTTTGCTGGCCAGCACCTGCAGTCCCCAATACCAATAC
CCCCCTAATCCTGGGGTACATTCCTTGCCTGAAACTAGAAAACTG
TCTAGTTTGCTCTAGCGCCCCCCTACTCTAGAGAGGGTGACAAGT
CCTTTTAACATTGGGTTTTCTTCACTCCCTCCCTCAGCTACAGCG
GCTAAAGCAGTTACTCAGGAAGGGTTCTACAGGGACAAAGGAGAT
GGAACTTCCCCCAGAGCCCCAGGCCAATGGGGAGGCAGTGGGAGC
TGGGGGTGGGCCCATCTACTACATCTATGAGGAAGAGGAAGAGGA
AGAAGAGGAGGAGGAGGAGCCACCCCCAGAACCTCCTAAGCTGGT
CAACGATAAGCCCCACAAATTCAAAGATCACTTCTTCAAGAAGCC
AAAG

In some embodiments, the intronic domain that promotes trans-splicing primarily in the muscle comprises or consists of sequences from the gene STAC3. In some embodiments, the sequences from gene STAC3 comprise or consist of:

(SEQ ID NO: 1182)
GATTACAGGCATGCACCACCACACCCGGCCATGTTAAGCTTTTTT
CTAGACCTCCATGAAATCTGTTAATAAAACCTGCCAAAGGATCTG
TATTTGGCCCAGTGCAGTGGCTCACGCCTGTCTCAACAATTTGGG
AGGTTGAAACAGGAAGATTGCTTGAGCTAGTTCGGGACCAGCCTA
GGCAACAAAGCAAGACCATGTCTCTACAAAAAATAAAAAAGTAGC
CAGGTAGTGTGCACCTTGTGTTCCAGCTACTCAGGAAGCTGAGGT
AGGGGGATCGCTTCAGGCTGGGAGGTCGAGGCTGCAGTGAGCCAT
GATCACGCCACTGCACTCCAGCCTGGGTGACAGAGTGAGACCCCA
TCTCAACAGTAACAACAGCAAAATCTCTCATCACATTTCTCCGAA
AGCCTCCCCTTTGAGGACCACCCTACTGCAAGGGACAGAAAGGGA
GAGAAGAATGAAAGGGATTAGATTTCATAGTGGGGTCCAACCTCT
TGCA

In some embodiments, the intronic domain that promotes trans-splicing primarily in the muscle comprises or consists of sequences from the gene BEST3. In some embodiments, the sequences from gene BEST3 comprise or consist of:

(SEQ ID NO: 1183)
GTAAGTGCATTTTTAAAAAATCTCTAATATTTTCCAGCCTGAGCA
ACATAGTGAGACCTTGTCTCTATAAAAAAAAAAAAAGAAATAAAA
TAATTTAAAGTAGCCAACTGTGATGTCACCTGTAGTCCTAGCTAC
TTGGGGAGGCTGCAGTTGGAGGATTGCTTGAGCCCAGGGGTTCAA
GGCTGCAGTGAGCTGTGATTGTGCCATTGCACTCCAGCCTGGGTG
ACAAAGTGAAGCCCTGTCTCAAGAAAAATAATTAATTAATTCACG
AAAATTAAAATAAATATCTCCAATATCTAGGAAATGAACAGATAG
ATCACTGTTCTAATCTTTTTACCTTTTTTGGCATTTTTTTCTACC
TAGGCTTAGTATGTAAGTCAGTCTCTGTTCAGTGGTTTTAACACA
GCAATCACCCCTAAGTATAATGAGCTTTAAATAAATGAGTTGAGG
CCTGAAGTAAATCAGGTACAGTTCTATAATAATTTCATTAAGTCT
GAGC

In some embodiments, the intronic domain that promotes trans-splicing primarily in the muscle comprises or consists of sequences from the gene BEST3. In some embodiments, the sequences from gene BEST3 comprise or consist of:

(SEQ ID NO: 1184)
TGAGCAGTATAGACTTCTTGTTATGTTTTTCCAAACAAAGAAATG
AACAAGAAACCAGGGCATATAAGAAGTGATGTCAAGTAAAAATGT
GTTTACAGGGTTAACAGAATGAAAAGCGGAATCTTCACCTTCCTG
TAATATTTGGGGTTAAGTAGGTGCTATCAGGTAAAACAACACCAC
AATTGTGAGCAAAGAATAAAAAGATGGCTCCCCTCCCTCCAGCTA
AGAGGCAACTCCACTCTAGAGTGTGTGGTTTGATCCCGGAGTGTG
GGAGGCTTCAAGCCCCACTTATCCTCATCGCCAAGTGCTCTCGAG
TAGTAAACAACAGATACAACTCTCTGTGGCAGCTGTGGTCATGGC
AAGTATGAACTACAGACATTTGTATATTCACTCATTTAGACTGTG
TCCTAGCCCAATCTAACTCTCATCTGCCCAAAAAAACTTCTCCTT
TTTATTTAATTTACATCTGAGTTATTCTAACAATATAACAATGAT
TTTA

In some embodiments, the intronic domain that promotes trans-splicing primarily in the muscle comprises or consists of sequences from the gene MYBPC1. In some embodiments, the sequences from gene MYBPC1 comprise or consist of:

(SEQ ID NO: 1185)
ATCTTGACTTTTGTTTCTTAAATACTGAGAGCAATTAATCATGAG
GTTTTCTGGGTAGAAATGAGTTCTCAGATAGTACATATATAGGCT
TTTATGTCTGTGTCCAAACGAATGTGATATATGAATAAGGTCTGC
CATTCACTTTAGTTACAAAATCAGGACTTGAAAACCACTTCCATT
ATTGATTTCATCAAGGTTTGATTTTTAGCATTCATTTTTTCCTAA
CCTACAGTCACATAAAAAATTTGCAGTGTTCCATTTTTAAATCTG
TGCTTCCATTTGGATTACCCTACTCTGTCAACTTTTTATTTATTA
TTTTTTCTTTCTTTGCCTGAAAAACTCAGGTTTCACTTTTCAGGT
ACCAAGTTTTCTAAGGCTTGCTCAGCAGTTGAAAGTGTATTGTGT
GGTTTCTCATCTTCTTTTTCAGATTTCTCTTTTCTCAGGTTACTT
GGGATGAAGTCTTTTCTCCTTGACTTTTTACTCCTATTTCTTGTT
TCAG

In some embodiments, the intronic domain that promotes trans-splicing primarily in the muscle comprises or consists of sequences from the gene MYBPC1. In some embodiments, the sequences from gene MYBPC1 comprise or consist of:

(SEQ ID NO: 1186)
TTGGGATAATTTCTACCCTTTTCCCTGCTTTTAAATATATTATTC
ATTGACGGTAGAGGAAAAGAAAAGGCAGTGAGCCTCCTTGCTGAG
TCAGTGCACAGAGCAGGGGCAACAGTTTTCGCCTGTGCTGGCACC
AAGAAGGAAATGTTGGCTTTAATGACAATTTTTTGTTTTACGTGA
GAAACTTCTTAGAAAGAGGCATTCTTTGAGAAATAAATAAGATTG
GTAAATCTTGGGTATAAATGTTAGGGGTCAACATATCTGTCTTCC
ATTTCTCACTATATATCCACCCTTTTCCCTGTTCTTTAAATTTAG
ATATTTGGATTTTAAAATTTTTCTTTTCTTTTTTGAGACTGAGTC
TCTCTCTATTGCCCAGGCTGGAGTGCAGTGGTGCTATCTCGGCTC
ACTGCAACCTGTGCCTCCCAGGTTCAAGTGATTCTCGTGCCTCAG
CCTCCTGAGTAGCTGGGATTACAGGTGTGTGCCACCAAGCTGGGC
TAAT

In some embodiments, the intronic domain that promotes trans-splicing primarily in the muscle comprises or consists of sequences from the gene COMMD6. In some embodiments, the sequences from gene COMMD6 comprise or consist of:

(SEQ ID NO: 1187)
GTAGCCGTTGCCATTGTAATGAAAGCTCCTCTTCCCCACCCTGCA
CTAGCCCGAAGAATGCAGATCAAGGATTGTTGGGCTTTGATTAGG
CGACAGTTATTCGAAGCCAGGAACACGGTATTTTAAAGTGTTGGA
TAAAGAAGTTAGGAAAATTTGCAGAGATAGCACCGCAAACATTTT
CTTAGTCTCTAAGTTATTTTTGAAGATGGAGTATAGCATTTTAGC
CTTTTAGAGTATGCATTTATAATGCTAGGAATATGAATTTGACTT
TTAAAGTATATCTTAGCCCCATTTCTGTTTCAAGTGCTGCTGTGC
TGTTGTAGCCAATAAACTATTTGTAGGCAAATAATTTATAACACA
CTTCCCAGAATAGGAGTACTAAAACTAGAGTATTTTTAGGCCAGG
AGTTACCCAAGTCCATACAAAATGTAAGAAAGGGATGAGGTAAGA
AATAATTAGAATACAGGGGCTGAAAGAGGTGTCCATTGACTGCTG
TCTG

In some embodiments, the intronic domain that promotes trans-splicing primarily in the muscle comprises or consists of sequences from the gene COMMD6. In some embodiments, the sequences from gene COMMD6 comprise or consist of:

(SEQ ID NO: 1188)
GGTTGGGATTGAGGAGCCGCGCAGCGGGATGCTGTGCCCCGCCCC
GCTCCAGTCTGGAAGAGCCCACGTGTCACTGTTAGATGGGGGAAG
CGGCATCCTAAATTTAATTAGCGCTTCTCTTCTCAAAGTATCTCC
CAAATCTTTGGCCTTTTTCCATAAAATAATTTTCTTTTAGCTCAA
TGTCTCCTAAGATATATGTTGAATTAAGAAAAAATATGAGTCACA
TAAAGATGCTTTTTTCAGCTTCCCCTGCTACGTCCGCCCCATGCG
CCAGGCCGCTAGGGGCAGGACGTGGAAGCGGAAGCGCTGGTCTGG
GGGCGTTCTCTCGAGTCCGGGCCGCAAGTCCCAGACGCTGCCCAT
GGAGGCGTCCAGCGAGCCGCCGCTGGATGCTAAGTCCGATGTGAG
TTCCAACCTGGAAGGAGAGTGGGCAGCGAGGCTCTGGAGCAAGAA
AGATGTTTCTCTAAATTGTGTTTCTGCTTCCGTTTCAGGTCACCA
ACCA

In some embodiments, the intronic domain that promotes trans-splicing primarily in the muscle comprises or consists of sequences from the gene SLC7A8. In some embodiments, the sequences from gene SLC7A8 comprise or consist of:

(SEQ ID NO: 1189)
ATCAACCTGCTGTTCCCCATCATCTACTTGCTGTTCTGGGCCTTC
CTGCTGGTCTTCAGCCTGTGGTCAGAGCCGGTGGTGTGTGGCATT
GGCCTGGCCATCATGCTGACAGGAGTGCCTGTCTATTTCCTGGGT
GTTTACTGGCAACACAAGCCCAAGTGTTTCAGTGACTTCATTGGT
GAGTTGTTGGAGGCTCTGGCTGGCTTCAGGGCCTCCTGGGCTGGA
ATGCAGTGGTCAGTGATGGGGTTGGGGAGCCACCAGGGCCAGGAG
ATCTAGGCTCACCTTCTCCTCAGTCCACAGAGCCCTGGAAGAGTC
CAGAGCAGGGGAAGCAGATGCCTTTTCCCAGAGACCTCTGAGGGT
AGAGAACAGCTGGCATTTCCACACAGACCTTAGACCCGCAAGAAT
AGGCAACCTGGGCTGGGCACAGTGGCTCACGCCTGTAATCCCAGC
ACTTTGGGAGGCTGAGACAGAGGATCACTTGAGCCCAGGAGGTTG
AGAC

In some embodiments, the intronic domain that promotes trans-splicing primarily in the muscle comprises or consists of sequences from the gene SLC7A8. In some embodiments, the sequences from gene SLC7A8 comprise or consist of:

(SEQ ID NO: 1190)
AAAATCTGCTAGAGGGAAGAGGAGGTAGAATAACCCCATGTCCCC
ACCACACACATCCCTTCTGAGCGAGGAAGACAAGGAAGAGAAAGG
CAGCCGAATCAGGACTGCCCCTGGAGCCCTGAAGAGGATACTCTC
AGGAACTCCCCACATGTGGCCCATAAGGAGCTGACATTTGCCATA
TTGTGCCCACATAAGGGCCCTGCACAGAAGTGGGCAAGGACCAGG
GAGCCTGAGTGACTCGAATCGTCTCCTGCCTGCACCTTGTGCATT
GCCACACCTCCCCTCCACCCTAGGACCTCAGCAGACACACCCATG
TGCCAGCAAGCTTCTCCATCCCTGTGGGCTTCTCTGGCTGCCACG
GGCACATGGCATGCATGATGAGGCCTTGTCTAACCACACCCAAGA
GCGAGGACATGGGCAAGGAGGATCGTTTCCTCCCTAACTCCTCCC
CATCCTCTTCCTGTCTGACCGACCATGTTCATCAACATTTGTTTA
TATA

In some embodiments, the intronic domain that promotes trans-splicing primarily in the muscle comprises or consists of sequences from the gene MYH7. In some embodiments, the sequences from gene MYH7 comprise or consist of:

(SEQ ID NO: 1191)
TCCTGACTCCAGACTCTTCTTCACTGCCCTGAGGACCCGGGGATG
GGGTCTGGACTAGGGGAGGTGGAAATGCAGGGGAGTGGGGGCAAA
TTCAAAGGCCTGGCCTAGGAGGCCTTCGTGGAGCAGCACTGAAAA
CCCCTTTCACTTCCCCCAAACCAGCCAAATGCAGCAGAGGCGACA
CCTCTCCTCCCAGCCTTCTCCCTGGTCCAGCCAGGATTTAAAGGA
GATTTAGGGAGACCCCTAAAGTTCACCTAGAGCACCCTGAGCCCA
TCTACTGTGGACCTGCACCCCAGCTTCCGTCAACCCTGCCCCACC
CACCACCATGGATACTGCCCAGGGCTGGGCAAAGGGCAGCGGTTC
GGGGTGTGGGTGCTGGCCAGTGCCCTTTCTGAGCCCGCAGAAGTG
TGAACCACTGGGCCAGGTGAGGCAATGGCTGCCTAAGTGCCAGTC
CCAGGCGAGTCTCTGAGAGTACACAAGGATTCACAGCTGTAGCCT
GCAT

In some embodiments, the intronic domain that promotes trans-splicing primarily in the muscle comprises or consists of sequences from the gene MYH7. In some embodiments, the sequences from gene MYH7 comprise or consist of:

(SEQ ID NO: 1192)
CTCCTGACCTTGTGATCCACTCGCCTCAGCCTCCCAAAGTACGGGATTAC
AGGCGTGAGCCACCACTCCCAGCTCAGCTCTAAGTTTCAGTGGTTCTGGG
AATTTCAGCCTTAGGGAGGGGTGCCACCCTAGAAGACCCTAATTTATATA
CAACCAGTATATCCCAGTTTCCCCCCAACTGCTAGTTTCCAGTTCCCTTC
CACCTAATTTTGTCCTGCAATCTTGTAACTCAAACCTAAAATGGACATCC
CAGAGAACCCGGGCCTGGCTTTGTTCTCGCCCAGGCAACTTCCCTCTGAC
CAACCCCAGAACCCTACCCTCCAGAACTTGCCCCCCGAACATCGATCCCT
GGAAACACATGTGCTGCTGCCACAGGGGCTTCCACCTGCCCACAAAGCAG
GCCCCCCACCATCAGACCCCTCTCACCTTTGTTCCCATGCCCTGTCCCTG
CCCAATACCATCTCTCCAAGGACTGATTGGACTTTGTTTCCTTTCAAAA

In some embodiments, the intronic domain that promotes trans-splicing primarily in the muscle comprises or consists of sequences from the gene CCPG1. In some embodiments, the sequences from gene CCPG1 comprise or consist of:

(SEQ ID NO: 1193)
ACGGCATGCACTGCGGCGCCCAGCCGGTCACCGCCTGCCGGCTGCAGACG
CCTGCGAGCAGGTTGTTTTTATAAGAGGCGTCATTGGCGCCCGAGCTGTG
ACCGCCGCCACTGGGGCAGCCAGCACAATCGGGCGGAGGTGGCGCTGCCC
CTTCAGGTACTGTACTAGGCGGGCTTAGACCCCGGGAGGGGGTTTGTGCC
GACTGTCTGAGTCCCTGGCCGACACGCTCGGCTCAGTAGAGGAGCAGTGG
CGGGGTCCGCCTCCTAGGCTGGGGCTGCTCGGGCGGGGACTTGACGCCTG
GTGTCAGCGGCCGCACGGGCGACCTCCTCGGGCCGTGCGCGAGTGCACAG
CTCCGGAGGCCCGAGCCGACCCTGGGGCGTCCGGTCCGGTGGTCTTGCAG
CCTCCAAACCCCGAGTGCTATACCGAACTGCGCGCCAAGGGTGGGAGAGC
TGACGGCCTGGGCCACCCTTCTTCCTTCACTGGGCAGGCTTTGAGGTGC

In some embodiments, the intronic domain that promotes trans-splicing primarily in the muscle comprises or consists of sequences from the gene CCPG1. In some embodiments, the sequences from gene CCPG1 comprise or consist of:

(SEQ ID NO: 1194)
CAGTAGGGTATGAATGCATACAATCTCGATGGTCTTTACACCTTCAGAGG
CTATTCGGACTTAAGGAAACGTGCATTTACAAGTGTAAATGTAAAAGCTC
AGTAGTTGCCTTGTAAAGTGCTTTTCGGGGTTGTGGGGTTGGGGGTGGTA
GGAGTAGGTGGTGCCTCAAATTATACTAGCGAATACCAACATTCCCAAAC
ATCTAGCCCTCTGCCTAAACTCACCCGGTTGCGGTCTGTCATTTTCGCTC
TTCTTTGTTAATTTTTTCGGCAAAGTCAGTTTTCATGCAGCAGATGGCAA
GAAAGCCGGTGAACGCCTAGGGACGTGGCCTTTTAACCGCACCCAATTAA
AGCAGCAAACAGAAGATGAATAGCTGGGTGGATCAGTATGTGTAAACCTA
TCCGTTTAATCCGGGGAGCTGTTTTCCCGGGTGAAACCACGAATTCTTTC
TGGAGGTTGCAACTCGTAACCATAGCAACTGGAAGTGGTCGCCGGCTCC

In some embodiments, the intronic domain that promotes trans-splicing primarily in the muscle comprises or consists of sequences from the gene NOMO1. In some embodiments, the sequences from gene NOMO1 comprise or consist of:

(SEQ ID NO: 1195)
GATTTCATAAGGACCTGTGTTCAGGTCGTGGTGGAAAGACAGGAGGGGGC
ACGTGAGACCTTGCAGAGGTGGAATCCACAGGACTGATCGATGCTGGAGG
AGCGGGGCGGGGCATCCAAAGTGATGCCGGGGTGTGCATGGGAGACTGGA
GGAAGACGTGGTTCACCAAAATGGGGGCATCTGGAGGGGAGGAGATGGCA
CCGGGGTAAGGGTGGGGGGCTTCTAGAAGCTTGAGTCAGTGACCTTTTAA
CGAGGGAAGTAAGTCTCTAGACTTTTCTGTTGTTTGACACTCAGCAGTGT
GTTGGGAACACAACAGAGAAGACAGATGTGGGTAAAGTGAGAGGGAGCCT
GGCTGGCACACAGGACCGGGGCACTGAGTGTTGGGAGGTGGGCGGCAGGA
GCAGACATGGTAGGTGGTGGCCGGCGCAGCAGAAGGAGTCTTTGTGGCTC
TGGCACAGGAGCACCTTCGAGCACCTTCTTCTTGTTCTTTGTTTTCTAG

In some embodiments, the intronic domain that promotes trans-splicing primarily in the muscle comprises or consists of sequences from the gene NOMO1. In some embodiments, the sequences from gene NOMO1 comprise or consist of:

(SEQ ID NO: 1196)
TGGGCTGACACAGCAGCCCCAGGCTGATGGCCAGCGCTCTTTTTGGATCA
CAGAGAGAAATGGAGAGGAACTGATGACTATATGAGAACATGGCAGCTTT
TGGTCCAAGTATTCTGGTGTACATGTAAGGATACACTGTTGTTTTTGGCT
TATCTTCTGTTTGTGTGTTTTTGTTTTACAGTTAAAACAAAAGCTGATGG
CTCATTCCGCCTTGAGAACATAACCACAGGGACATACACCATCCATGCTC
AGAAAGAGCACCTCTACTTTGAAACGGTCACCATCAAAATTGCACCGAAC
ACACCTCAGCTGGCTGACATTATTGCAACAGGGTAAGCTTATCGTGTGGA
TTTGGAAGCGCCAGTAAATATGCTGGCAGCCAGTGTAGAACCGAATGACC
TGTGATCTGTGTGTCTTTGCCGAGCTTAAGAACTTAAGAAAGATTAGTAC
TTTTTTTTTTTTTTTTTTGAGAGACAGAGTCTTGCCCTGTTGCCCAGGC

In some embodiments, the intronic domain that promotes trans-splicing primarily in the muscle comprises or consists of sequences from the gene NOMO2. In some embodiments, the sequences from gene NOMO2 comprise or consist of:

(SEQ ID NO: 1197)
GTGAGACTTGGAATGTGTTTTCTTTGGGGACTTTTTTTTCATCCTGTGTC
CAGAAGTATCTTGTGGTGGCCCCAAGACTGCTAGGAGTGGGTTGGGCGAG
AGGGCTGGGGGTGGGGCTCTGGAGCCCTCCCAGTTCTTACCTTTAATTGG
TAGAGTTTGCCTTTTTTCTAGCCAACAGGTCAGATCAAGGATCTGTGGCA
CAATGGAGTTGGAAAACCATGGGTTTGGTAGGGCTTTTCCCTAGCAGTAT
GTGGCCATGGCTTACGCCAGGGCTCAAGCCAAACGCCTATGGAGCCAGAA
GAGCAGTATGAACGGGGGAGAGGAGGCTGTGCGAAATGATAGGGAGTGGT
GAGGGCTGTGGAGTGGGCACGGCCTGTGGAAAGGGACAGCCTGTCACTTG
GTTCCAGCTTATTGTAGCCATGGGGGAAGACAGAGTCAGGGTTGCCAGAT
CCTTGAAATTTTAAAAAGAGAAGTCAGAAATTCCCACTTCCCAGAAATA

In some embodiments, the intronic domain that promotes trans-splicing primarily in the muscle comprises or consists of sequences from the gene NOMO2. In some embodiments, the sequences from gene NOMO2 comprise or consist of:

(SEQ ID NO: 1198)
AAAAAAGATACACATCTATAGATAGAGAGATGCATACATATATACATGTA
TACACACAGATACACACATATGTGTATTTTTTGTAGACTTATACAGAATC
ACTTTAGAGGAAGAGTAATTGGAAAGATTGGGTTTAGAAAAATGGACCAT
TTTCTACTGAATATATGGAATGAAAGAGGAAGTGAGTTATTTCCATGTTA
TTGAAAGCTCCATTATTTCAAATAAAGCCTAGAAAGAATAAAATAAAGCT
TTAGCCTTCTTAAATTACAATTTTCCAGAGCATCCCTCAAAATGTATGAG
AAAAGAAATTGATGGCAGCTTATCCCTCCGGCAGGAAGGTGTGGTTTGAA
AGTGGGCCGGCCACACCGAGTTGCCTGGGTCATTGAGGCACAGTTCTCCC
CTGAACTATTTTGCCCCATAATTGTTGGTAAACGGGAAACACTTGGGTCC
TTCAGATAACCATGAGAATATCTCATTCGTGCCTGTTCTTGCCATACTA

In some embodiments, the intronic domain that promotes trans-splicing primarily in the muscle comprises or consists of sequences from the gene ATP2A1. In some embodiments, the sequences from gene ATP2A1 comprise or consist of:

(SEQ ID NO: 1199)
CTCCCAAAGTGCTGGGGTTACAGGTGTGAGCCACCACGCCTGGCCCAAAG
ACCCCTTTTCCAAGTAAGGTAACATTTACAGGCTCCAGGGATTAGGATCT
GGTATCTTTGCGGACCATTATTGTTTGTTTGTTTAATAGAGACAGAGTCT
CACTCTTGTTGCCCAGGCTGGAGTGTGGTAGCACAATCATAACTCACTGT
AATCTCGAACTCCTGGATTCAAACAATCCTCTTGTCCCAGCCTCCTGGGT
AACTGGTACCACAGGCAATCACCACCACACCCAGCTAATTTTTAAATATT
TTCAGAGGAGTCTCACTGTGTTTTCCAGGCTGGTCTCAAACTCCTGGCCT
CAAGTGATCCTCCCGCCTCAGCCTCCCAAAGTGCTAGGATTATAGGTGTG
CACCAACGTGCCCAGCTGGGGGCTACTATTTAGCCCCTTGCAGGTTCCCT
CACACCCTCCCCTTGCAGGTTCCCTCACACCCTCCCTCCCTCCCCACAG

In some embodiments, the intronic domain that promotes trans-splicing primarily in the muscle comprises or consists of sequences from the gene ATP2A1. In some embodiments, the sequences from gene ATP2A1 comprise or consist of:

(SEQ ID NO: 1200)
TCAGGAGCAGTGTGGGCAGCGCGCTCAGTCAGAAGGCTGCCTGTGGGGGT
TAAATGGGCCCTCCAAAGATAGAGGTCTCCCTTCATCACTGCTGGCTTCT
CATCTGGGCCTGCAAAATGCTCAAAGGGCAGTAGAACGCCATCCTGTCTG
CCATGAACGGGATCAGAGAGGACCCTTGTGCCCCAGCCAGACAGCTCACT
CTGACCACCATCCACGCACCAGGCACTGCCACACATCGTCTCTCATCCCT
CACAATAGCCCATTCAGAGGATGGTCTCATTATCTTCATTTTTTTAATTA
AAAAAAATTATTATTATTTTGAGACAGAGTCTCACTCTGTCGCCCAGGCT
GGAGTGCAGTGGTGTGATCTTGGCTCACTGCAACCTCTGCCTCCCGGGTT
CAAGCGATTCATCTGCCTCAGCCTCCCTAGTAGCTGGGACTACAGGTGCC
TGCCACCACGCCTGGCTAATTTTTTTGCATTTGTAATAGAGACAGGGTT

In some embodiments, the intronic domain that promotes trans-splicing primarily in the muscle comprises or consists of sequences from the gene ALDOA. In some embodiments, the sequences from gene ALDOA comprise or consist of:

(SEQ ID NO: 1201)
GGACTCTTGGCCGGCTGGACGGCAGCTCCTCTGGAGGGCCAGAAAAGAGA
GGGGCTAGTGCTCGGGCAGGTGCCCTGGCTTCCCTTCCCCTCCACACGTC
AACGATTCTATTTGAAGTTGGGCAGGGGGGTGGCGCTGCTCACCACACAC
AAGTGTTATAGGAGGAGTCTGGCCCTTGAGTACCGGGTACGCAGGGGTGC
CTCAACCACACTCCGTCCACGGACTCTCCGTTATTTTAGGAGGTGAGTGT
AGTGCCAGTATCTACTCTCCTTCTTAAAAAAAACCAGGGCTCCAGAGAAT
CAGAACAGCCACCATCACCGCAGGGAGTCAAGGGAGGAGGGAGATTAGAG
AAGGAGCCAGGGAGGGTGGCAGGGAGGCCACGTGATCCGAGTCCCCTCAC
CCCTTTCCTTCCCACAGGTCCCTGGCCAAAGATTTATTTCTCTTGACAAC
CAAGGGCCTCCGTCTGGATTTCCAAGGAAGAATTTCCTCTGAAGCACCG

In some embodiments, the intronic domain that promotes trans-splicing primarily in the muscle comprises or consists of sequences from the gene ALDOA. In some embodiments, the sequences from gene ALDOA comprise or consist of:

TGGGCCCCTTGCAGGACCGGGCCGGGGTGGGGATGGGGTTGGGGTTGGGC
CAACAGGGTCCAGATGGGGTCCAGGTGAGGAGGGGAGATTTGGACGATAG
GAGCAGGGGGCTCAGCATCTGGGAGGCAGATCAGTTCGGGGACGGATTTT
CTTTTGGAGAAGGAAGTCAGGCTCAAGGAAGACGTTTGGCAGGAACTGTG
ACCCCGCATGCCAGAGGCCGAGCAGCGGCCGGTGCATAGCCGCGCATTCT
GGTTTTCTGTGGCGCAGAGGACTACCAGCCTGGCTGCGGCGGCCCGGCGG
AGAGCGCGCACGCATGCGCGACCCAGCCCGGCCTGCCAGCCTGGAACTCG
GATGGGGAGGTCTCGCCTCCGCGCGCCGCTAGTTCCGCCGCCTTCT

(SEQ ID NO: 1202)
CGCGCTCCCTGCGTGTCCTCTCGATGCCCTTTCCTCCGCCTCCCTTACGC
CTGGGCCAGTGACAGGGTGTCGGCTCCGCGCCGATTCAGCCCGCGGGCGA
GGC

In some embodiments, the intronic domain that promotes trans-splicing primarily in the muscle comprises or consists of sequences from the gene CHRNB1. In some embodiments, the sequences from gene CHRNB1 comprise or consist of:

(SEQ ID NO: 1203)
TCAGCTGGGTTTTAGGTACCCAAAAAGGGCCTGTCTAGATTTTTTCAGAA
AAACGTGGAGTGCTAGGGGCAGCCTGGAAAAGATGGGGAACCTGCTAGTG
AACTAGGAGGGAGACTTCCATAGCCTCAGACTTGGATAGGGTAGGCTGAG
GGGGCCCTAAGGGAGGGACTAAGGCTCCAAGGCAGGTCACTTTTCCTTAG
GCTGTTCTACTTCTGGCTTGTTGCAAGAGGAGTAGATGCCCCCTCACCCA
CACAAACCCCACTCAGTCTCCACCCAACTCCTGGCACTGCTCCCAGGGGA
TCGGGTCTCCACTCCAGCTTTCTCAATTAAAGACGATTTATACAACTGAA
GTGCTGTCTGTCATCTGTCGGTGGCGGGCTTGGCAGTTGCTCGAGGCAGG
ATCAAGTCCCGGGGGCGGGGCGGGTGGGCTGGCGGCTGTCCTCCAGCAAC
AGGTGCACATTCCCGGGCTCCTCGTCACTTCCCCTGTGCTGGCGGTCCC

In some embodiments, the intronic domain that promotes trans-splicing primarily in the muscle comprises or consists of sequences from the gene CHRNB1. In some embodiments, the sequences from gene CHRNB1 comprise or consist of:

(SEQ ID NO: 1204)
TAAGTGTAGGCCCCGAAGGGGCAGTGACGGGGCCAGCGGTCGTGGCCAGG
CACCAGGGCTGCACTTATTCTCTCCTCCCCCAGGCGTCCGCGGCTCGGAG
GCGGAGGGTCGACTCCGGGAGAAACTTTTCTCTGGCTATGATAGCTCCGT
GCGGCCAGCGCGGGAGGTGGGAGACCGTGTCAGGGTCAGCGTTGGTCTCA
TCCTGGCGCAACTCATCAGCCTGGTGAGGGCGCGCGGGGGGTGGAGGTCA
GGCCAGCCGACCGGCCGGGGGCGTGGCTTTAGGCAAGGCCGGACCAGGGA
CAGGCTGGGGGCGGGGCCTGGGACGAGACCAGGCTGAGATGGACCAGCCT
TTGGTGAAGATTGGATCGAAATCAGACCAATGGACAAGCTCTGGCCGTGG
GTGGTGGACGGGCCTGGAGTAGAACTGGGTAGGGTGAAGGACGGACCTGT
GATCGGACCTTAAAGTGGGGCTGGGGACGAGGCAGGGGCTGGGGGACGG

In some embodiments, the intronic domain that promotes trans-splicing primarily in the muscle comprises or consists of sequences from the gene DHRS7C. In some embodiments, the sequences from gene DHRS7C comprise or consist of:

(SEQ ID NO: 1205)
GTAAGCTGGCTTTGCTCCCACCTGTGCCCATTTCTGCCCCCAGGGACTGG
CAGGATGGCAGGGTGGCAGGGCGGCAGGGCGCTGTGTGGGCAAACCTGCT
CGGTGGGAGGGGATCGATCCTGTGTTCTCTCCCAGCTTCTATCATTAACC
AGCAAGTTTCCCATCTTTTGGAGGCTGCAGATTCCTGATAAATGAGGGTG
GTGGAATATCTAGGGTTGCAAACTCACAGCCTAAGGGCTAAAACTGGTCT
GCAGATGTGATTTGTTTTGGCTTCATGGGGTTTCCAGAAATTTTGAAAAT
TCAAAGCCTTTAGCTGGGGCAATGTACTGGTTGTGAGCCCAGACTCAGGA
GCTGGGTTCAGTTCCTGGCTCCAGCAATGACTAGCTGGTTACTTCACTTC
TCTGCCTTAAACTTCTCATCTGTAAGGCGAGGATAACAGCAGTCTCTACC
TCATGCTGCCATTGTGAGGAATAAACGAGTTCATAGATGGATAGCACTT

In some embodiments, the intronic domain that promotes trans-splicing primarily in the muscle comprises or consists of sequences from the gene DHRS7C. In some embodiments, the sequences from gene DHRS7C comprise or consist of:

(SEQ ID NO: 1206)
AGGATGTTTGTGCTGAAAATCCCAGGTTTGGATCCCTCGTGCTCAGTCC
CAAACTGCAGAGGAAACCTCTAATACTCAGGCAAGCCAGGAGAGCTTTG
GCAAGAGCCCTGAAATCAGACCCGGCCAGCATGTGTTCCTGAATATCAG
AATAGACACTATATTTACCCTGACAGCCTTTTAGTGCAACTGAAAGCTA
TAAATAACCATTAGGGCCCAAGCAAGTGGCCTTAACACATGGATTTTCT
TCCAAAAATGCAGACCCATTTTAATTAAGTTTGTAATTAACCACTGGGG
AGGGCAGGCCCCCTGGATTCGGTCTGCTTTCGGAGACACTGTGAGTAAC
TTCCTATTTGTTGAACATTTGGGGATTAGCACGCCCACTGGGTGTTCAG
CTTGGAGGCTTGCACAGAGCTGAGCTCCCTGCAGCCTTGGGCCTCCCCC
TGCCCTGGGAGTCCTGATCAGCGTCTCTTTGCAAAGCCAATCCCCTTTT
ACTCCGTTG

In some embodiments, the intronic domain that promotes trans-splicing primarily in the muscle comprises or consists of sequences from the gene NPEPPS. In some embodiments, the sequences from gene NPEPPS comprise or consist of:

(SEQ ID NO: 1207)
GTGCTGGGATTACAGGCGTGAGCCACCACGCCTGGCCACAACAAAGAAT
TTTACCAGAAGTGGGTATGATTTATATCTTAGTTTGAATTGCTACTTAC
CTTTGGGGGAACAGACCTTTCCTTGATGTATCTTTAACACTGAAATCTT
GGCAAGTTGCCAAGTCTCAAATGTTCATGAGTAAGACTGCAATATCACA
ATATCATAGTCACACAGGAATCTGATAGAAAAGCATCACATTTATAAAG
CCTTCTTTCCTGTACATCACTGACGTTTTGTGAATTTTAAAGAATTTGT
AATTATTTTTAAGGAGCATATTTAATGTAGATAATGTAGCCTAGAATAG
GCTCATTTGAAATGAAACTCTTGCTAATAGGAACTTAATTCACCAAATT
AAGAATATTTTGCTTTTGTAGAGATTTTGCTCTTGAAAATGTTGCAGTC
TTGATTTCGTCTTGTCAGTCCAGTCAGAATTGTAAAGTAATTTTTTTTC
TCATTCCAG

In some embodiments, the intronic domain that promotes trans-splicing primarily in the muscle comprises or consists of sequences from the gene NPEPPS. In some embodiments, the sequences from gene NPEPPS comprise or consist of:

(SEQ ID NO: 1208)
TAAGAGACATAGCTTTTGTAAAATCTCGTGATGAATATAGTGACATCTG
ACTTCCTCCAGAGAAATTTATTGTATGCCATTTTTCCCCTCATTGTTAT
TAGCAGATTAAGTATTAAGAGCTTTTTTTTTTTTTTTCCTTTGAGACGG
AGTCTTACTCTATTGCCCAGGCTGGAGAGTAGTGGCATGATCTTGGCTC
ACTACAACCTCCACCTTCCAGGTTCAAGCGATTCTCCTGCGTCGGCCTC
CCGAGTAGCTGGGATTACAGGTGCCTACCACCACACCGGCTAATTTTTT
TGTATTTTTAGTAGAGACAAGGTTTCGCCATGTTGGCCAGGCTGGTCTT
GAACTCCTGATCTCAAGCGATCTGCCCACCCTGGCCTCCCAAAGTGGTA
GGATTACAGGCAGGAGCCACCGCGCCCAGCCGTATTAAGAGCTTTTTAA
AGAGGTTTGTTGCAACTTTTGGGCCACCAGCATTTCCTTCTGCGGAAGT
TGGGCTACG

In some embodiments, the intronic domain that promotes trans-splicing primarily in the muscle comprises or consists of sequences from the gene KPNB1. In some embodiments, the sequences from gene KPNB1 comprise or consist of:

(SEQ ID NO: 1209)
GGCCTGAGGGCGGCGGTGCAGCGGGAGGGAGAAAGAGGGAGGGAAGGGA
AGTTAGGTTGCGCGGCGCTGCGTGTTCAGCAGGGGTGGGGGCGGGGGAG
TCGGGCCTAGGGGCCATGTGGAAGCTCCGGGCCCGGCCGCCCGCCCTGT
CTGACCCCGGCCGGCTTCTCCTTCCTATCCCGTGGCCCCTGTGGCTCTC
CCCACCTATTTCCTAGAGCTCTGCCCTTTCCTAGCTACCTTGGTCTGTA
CTCATGGCCAGGCCTGGCACCCGACTGCCTCTTAACCCATTTCCACTGA
TATTTTTCCCTCTTTGCTCGGTTATTCCTCTCTTGGGGTTTTTTCTTTT
TCTTTCTTTCTTTTTTTTTTTTCTTCTGACCTGGATGAATAGAGACCTA
ATCACTAGGGATCTGCACCCAGAAAACAGACTGAGGCTCATCTTTGAGA
TGGGGCCTGTTTGCTGATGAAATCACAGGGGAAAGTTCCTCGTTTTTCA
TATTTTAAA

In some embodiments, the intronic domain that promotes trans-splicing primarily in the muscle comprises or consists of sequences from the gene KPNB1. In some embodiments, the sequences from gene KPNB1 comprise or consist of:

(SEQ ID NO: 1210)
TAATAGGAATTAAGTCTGAGTAACACTAACCATATGTGTTCCTTTGTGT
TATATAGGCAAAATGTCAACATCAACAAAGTGACAAGGCTTATTTTATA
CAGACTGCCTTTGAATGATTTTTTTGTTGTTTTTCATATGTGCCAATTT
TTACAACTAACCTGTTTATCCTTTCCTAGAAGAGATTTTTCCACTAGTA
TAGGCTGATAATTCCATTTAGGGTATTATTCCTGTTTCTGCTTTTGCAA
ATTTTGCAAAAAGCTTTTTATCTTATTCCTAACATTCTTAACATTCTTT
AGTGGTACATAATTAATAAAACTTTTAAGAATTATCTTCTGCTGTGATT
GCAGAGCTTGTTAGCAGTAAGAAAAATGTGTCTGCTTTTGACTTTTCCT
GCTAACTTTGCTGTTTGGGCACGGTTTTTGGTGGAGAGGTGTGTGTGTG
CTAAATTTTTAAACAAAGGGAGTAGTTTTTGAGGCCTGTGGATTACAAA
AGGTTACAA

In some embodiments, the intronic domain that promotes trans-splicing primarily in the muscle comprises or consists of sequences from the gene SPAG9. In some embodiments, the sequences from gene SPAG9 comprise or consist of:

(SEQ ID NO: 1211)
GTAAGGCTCTTCTCAAGGACAAAAAATATGATATCGTAAGCTGCATCAT
GTTGACTGTGCATTTGTTGCAATCCTCTGCCAAATTTAAATAATGGGGA
ATTATTTAGAGACTTGAAAATGACCTGGATCCCCATTAAATAAGTTTCT
GATTGGCTGGTTTTGTGATTGTAAGAAACTTTACAAAGTGAAGATGAGA
AATTTAGTGCTTGTAGGGTTTGAGTAATATTGTGTATGTATTGAGCTTG
AGTTATGTTTGTGCTAGAAGTACTATGTGTTTACTGTGATTAAAACTTA
CCCTTTTTTGGTGAATCTGCTATTTAATGGAAGTGTCAATTAAAAAAGA
AATAGACCCGAAAGTAGTTGTTTAAATTGAGCTTAATCAGAAAATGTAT
AAAATGTGTGTTAACATGGTAATATGTGTTCCAGTACTTGATTATAATA
AGCTGTTTTTAGAACTGGCATACTAAGCTTCCATTCACATCACACAGCC
ATAACCTTG

In some embodiments, the intronic domain that promotes trans-splicing primarily in the muscle comprises or consists of sequences from the gene SPAG9. In some embodiments, the sequences from gene SPAG9 comprise or consist of:

(SEQ ID NO: 1212)
TGTTGGCTGTCTAGCACATATTACAGTCCTCTGTCGGTAGACTAAGCTC
TTTGAGGGAGGGATGATATTTTATTTTTCTTAGTAGAATTAAAGTCAAT
CTTTAGCAGATTGACTGACACATGGTAGGTCAAAGTAACTACAGTATTT
ATTGAACTTAATTGTTGACTCCCCTCTGTGTATCCCATGAACTTCTTGG
GTCTCTTGTTCTTCAGAAAGAAAGGTAGAATACCTCACTGATAAATGTT
TCTTTTCTTTTCTTTTTGAATGTAGGGCATCACGAGAAAATCCAGCCAT
GCAGGAAAAAAAAAGGTCAAGCATTTGGCAGTTGTAAGTATTGAAAAAC
AAAGATGTTAGACAGCACATGAGACAAGAAAATTACAGTCAAAATTTAT
TTCCAAGAATGATTTTATGTCCATTTTCTTTCCTTCATTTTTTTTTTTT
AAACTAGGGGTAATGGTTAAGTAGCTAAACTGAACTTTTTTTTTTTTTT
TAATATATA

In some embodiments, the intronic domain that promotes trans-splicing primarily in the muscle comprises or consists of sequences from the gene MAFG. In some embodiments, the sequences from gene MAFG comprise or consist of:

(SEQ ID NO: 1213)
GTATTGGGGGCTGGGCACCTGGACAGCAGGCTGGGGCGGGCTGTGCGGC
AAGGGCTCTTGTGAACTGGGCCGGGGGCGGGGGGGGGGGGGTGCGGCGA
GGGTGGTCTCCAGGGTACTGACCTGCTCCGTGTCTTGCAGAGAGCGCCT
GCTCGCTGTGCCCCCGGGTTATGACGACCCCCAATAAAGGAAACAAGGC
CTTGAAGGTGAGTGGGCAGGGACAGGAGCCTGTGGGCTGGGGTCGGGGG
GCACAGTGGGCGTGCCCGCTTGGCCTGTGACCACCTGCACTGTGCCCCA
GGTGAAGCGGGAGCCGGGTGAGAATGGCACCAGCCTGACGGATGAGGAG
CTGGTGACCATGTCGGTGCGGGAGCTGAACCAGCACCTGCGGGGCCTGT
CCAAGGAGGAGATCGTCCAGCTGAAGCAGCGCCGGCGCACGCTCAAGAA
CCGCGGCTACGCTGCCAGCTGCCGCGTGAAGCGGGTGACGCAGAAGGAG
GAGCTGGAG

In some embodiments, the intronic domain that promotes trans-splicing primarily in the muscle comprises or consists of sequences from the gene MAFG. In some embodiments, the sequences from gene MAFG comprise or consist of:

(SEQ ID NO: 1214)
AAGCCACAGGATGGTTACTCCATCAGTGGAGGAGGCCGACTGGGAGGTC
TAGAGGGGCCTCGGGCCAGGCTCCTCCCAGCTGATGGCCTGCCACCTGG
AAGGTGGGGGAACTCCCTGGTCAGTCCTGCCAACAGACCAGAGCTAGAT
GGACGGCCCACCCCCAGCTTTTCAAAGTTAGGAGGCTGGGAGACTCGGG
GTTCCCTGTAGCCTCCTCTCTGGCCAGGTCCCCCGTGGGGCCCCTGTGC
ACCCAGGGAGCAGGGGGCTTTGTCCGAGGCCGGCTCAGGGGAGGTGGCT
GTAGAAGGTAGGACCCTGGAAGATGCACACACAGGGTCACTGGCTGGCC
CTCACCTGCTATTGGCCAGGCTGGGGGCTGCGGCTCTCGTGGGGCGCTG
TTGGCTGGCGGGCCAGGGAGTGCGGGCGCGGCGGGGAGGCAGAGGCAAT
GTGCTGAGTCAGCGTGACTCGCCAGGAACAGCCGCGCTCTGGGCAGTGC
CGGGGTTAT

In some embodiments, the intronic domain that promotes trans-splicing primarily in the muscle comprises or consists of sequences from the gene RYR1. In some embodiments, the sequences from gene RYR1 comprise or consist of:

(SEQ ID NO: 1215)
GTCTGTGAAGCAGAGGTCAGAGGTGTTTGAAGGAAAAGGCAGGAGCGAG
GACTGTGTGATGGTGGAGGCAGGAGGTGGGGCTTCCTTTGGGGTTGGAG
GGATGTGGGGTCATCTGCAGAGGAGGACACAGAACAGGAGATTCCTGTC
CTTGGAGCAGGGTCAGTGTGTCTCAGGGGAGAGGCATGGGGGTGAGGCT
TCCTATGAGATTAGGTGGAACACGGGTTAGTGGGGAGGGTTAGGGTTAG
GGAAGTTTCTATGGAGTCTGGGTGGGTCTGAGGTGTTTGAGGGAGGAGG
CACAGAAGAGGTGTTTTCGTGATGTTGGTCTAGATTCAGTGTGTCTGAG
GGGGATGGCAGAGGGCAGGGCCTTCCTGTGGGGTGGGGGTGGGGTCTGG
CGTCTCAAGAGTGTGGGCATCCAGACTAGGGGAGGGAGTGTGGCAGGGA
ATGTTGCTGGGGTGGGGGGGTCTTCTGACCCCTCACTTACATCCCCCTC
CCACCCCAG

In some embodiments, the intronic domain that promotes trans-splicing primarily in the muscle comprises or consists of sequences from the gene RYR1. In some embodiments, the sequences from gene RYR1 comprise or consist of:

(SEQ ID NO: 1216)
TGAGGACCCCACCTGGGGGTGGGCGGGGTGGCAGAGATGGGCGAGAGGA
CCCAGGGGTCGTTTAGGGCACGGTGGCAAGGATGGGTGAGAGGACCCGG
GGGTCGCTTACCATCTGCTCTTCTTCCTCTCTCTGCCCTGCCCCCCACA
GGCCCTCAATTTGGATAAGTGGGTAGGTCCGGCCTTGGATGTCACCTGT
CCTTCCACCCCTCCTGACCCCAGGCAGGCCCGTCTCTCAGGCCCACGCC
CTGCACACAGGGGCTGGAGGGTCCTCCCCCACCCCAGTTGTCAGGGGCT
GTGGAGCCCAGAACAGGCCAGGCAGGAAGGAAGGAAGGGGATGGGGGCT
TCTGAGCCCCTGTCCCCGACGCCTGCTCCTTTCTCCTCTTTCCAGTCCT
TTCCCCCAACCCTGATCCCAGCTCCTCTCCGCATGGGACCTCTCCCACC
TCCCCCATCCCAAGCGTGAGTCCCTCTGGTCCAGGAGTGGGTCCACCAT
GTGATCACC

In some embodiments, the intronic domain that promotes trans-splicing primarily in the muscle comprises or consists of sequences from the gene MYLK2. In some embodiments, the sequences from gene MYLK2 comprise or consist of:

(SEQ ID NO: 1217)
TGAATATCCCCTCCTGGGAGTGGGGAGGGGTCCTGTGGTTCTGTCCCTA
GGGGTCCTGCTTAATTCCCTTGTCTTAGGCATGGTTATCATCACATTCA
GTGCTGGGGAGTGTAGTTCTGACATTCAGTTTCCTCTGTCCTGTGAATT
GCCTCTACCGCCTTGTCCCCTGCAGAAACCAGAACCCTGCCACAATTCA
TACAAGACTGGAGTAAGAGAGACCCTCAAAGACTGCCTAGGTTGAACAT
ATCTTCTCTAGGCTTCTGGGGTCCGAAGTTCCCCTTACTCCATGGGCTT
GGGTCTAGCCAAGGGCAGGGTTGAGATGGCAGGCAGAAAGGCTAAAGGT
GTCAGGCAAAAGGCTGTGTATTTCCTAGGACATCCAGTATTTTCCTGTT
CTTCTTAATATAATTATTGGTAAGATATGAAAAGATGACTAATAACCAG
ATTGGAGGATCATTGATAATATTTTCATAACCATAATATAGCACATGTG
TGATGCCAT

In some embodiments, the intronic domain that promotes trans-splicing primarily in the muscle comprises or consists of sequences from the gene MYLK2. In some embodiments, the sequences from gene MYLK2 comprise or consist of:

(SEQ ID NO: 1218)
CAACAGGTGCCAAGCTGGGGCAGGAGATGGAGGGAGGAGCTTGGGAAGG
GGGGTTTTGAATCCAGGACTGGGCAGGTTCCTCAGTGGGAGTTCTGTGC
CCCAGCCTTGTTTGCATGGTGCACGGGGGACCCAGAAATCAGAGGAATC
TCTGGCTAGGGTTGGGCTGGGTCCAGGGCCTGTATGAAGCCCTTCCTGG
AGAGACCAGGAAAATGTGAAGCCACCAGGGATGGCTGCTGGGGATGCAC
CTAAACAGTCAGGGCAAGGATGATGGGGGCCACCAAGGCCCACCCAAAG
GGCTGGAAGCCTCCCACAGAGGCCCTCCAAGGTCGTGGCAGCTCATCTC
TAGGGGCCATCAAGTGGCCCCAGAGACTCAGTGGGACAAGCATTTGCAA
GTTGTTGCCTGGGTGGGAACCTGGGCCCAGAGCTGGTCTCTGCCCAGCC
TGGGTGAGAGGGAAGGAAAGGAGGGTGGATCCTGATGGGTGTCTCACCT
CCTCTGCAG

In some embodiments, the intronic domain that promotes trans-splicing primarily in the muscle comprises or consists of sequences from the gene CLTCL1. In some embodiments, the sequences from gene CLTCL1 comprise or consist of:

(SEQ ID NO: 1219)
GTAAACCCGATGGGATTGTTAGAAAGGAATCTTACCTCATTTTAACTGC
TTTGTTACCTTAAATATTGAACAAGTGTCACTAAGCTCATGGCGAAGTG
CTCCTCAATAGTATTCCACTTAATTCCAAAAGTTTTAGCAAAAGTTATG
TTTAGTGTGCCCTAGTAAGAGGCCCCTGAGCACCAGAGCTTCTTGGTAA
CATCACCCACAGCGCCAGACACACCGAGAGGAAGAAAATGCCCAGGCCT
TTGTCTTCATGCGACTAGGAACGCACAACAGCACTGTGCATGTTTCCCA
GGAAGGCTGTGTGGCCCCCTTGCCTGAGGGCCTGCTGCAGTGGTGCTTC
CGGTCATCCGACGACACAGGCTCGGGGGTTTTCCTCCTGTGAAACACAT
CAATGATAGAGGAGCTGATAATTAATAGAATTTTACTTCCTTGAAATCA
GGGAGTCTTGGGTGCTTAAAGCCTTCTAGTTATCTTGAAAAATATAGAA
TCTTCTTAT

In some embodiments, the intronic domain that promotes trans-splicing primarily in the muscle comprises or consists of sequences from the gene CLTCL1. In some embodiments, the sequences from gene CLTCL1 comprise or consist of:

(SEQ ID NO: 1220)
TTGATGATATCTAGGGAAATGTTTAAGCAAAAACTTTTGGCCAGTTCTT
TTTTCTTGCCTTTTGGAGAAATCAGGTTTTGTGTTTCCTCCCTTTTTCC
CTTCCACTTCCCTCGCCTTCACCTCTCCAAGACCTCCTTCCTCCCAAAG
CTCTCCTCGGTGGTGGGCTTATCTGTGACACCTTATCTGATGGTGTTCT
TTGGGAGTTCACCCCCAACTCATCTCCTCCTGTCTGTATCACTTGTGGC
GTCCTTGACACTGCCTAGTGTCATCCTGCACATGTCTTATCTCCACTGT
GGGCTGGTAATCTCTCAAGGAGAGTTGATTCACAGGCTTGGGTGGATCT
ATTACAGGCGTAGAAAAGCAAGCTAATGTTTCCTTCAAGTGTTTACCTA
ATGTTAACTTTTTTCTAAATTTTATTTAACTTCTCCTCTTTTGCCTTTG
GCTACAGTCAGTCCATTTCAGAGGAAAAAAATTTAATCAAATGCTTTTT
TTCTGTTTA

Replacement domains

Compositions comprising replacement domains disclosed herein include any strategies where replacement or insertion of RNA sequences could be an effective therapy. Exemplary replacement domains include without limitation sequences derived or isolated from the following genes (with gene accession IDs in brackets and associated diseases in parentheses) such as TNFRSF13B [ENSG00000240505] (common variable immune deficiency); ADA, CECR1 [ENSG00000196839, ENSG00000093072] (Adenosine deaminase deficiency); IL2RG [ENSG00000147168] (X-linked severe combined immunodeficiency); HBB [ENSG00000244734] (Beta-thassalemia); HBA1, HBA2 [ENSG00000206172, ENSG00000188536] (alpha-thassalemia); U2AF1 [ENSG00000160201] (myelodysplastic syndrome); SOD1, TARDBP, FUS, MATR3, SOD1, C9ORF72 [ENSG00000142168, ENSG00000120948, ENSG00000089280, ENSG00000015479, ENSG00000142168, ENSG00000147894] (Amyotrophic lateral sclerosis); MAPT, PGRN [ENSG00000186868, ENSG00000030582] (Frontotemporal dementia with parkinsonism); CDH23, MYO7A, USH2A [ENSG00000107736, ENSG00000137474, ENSG00000042781] (Usher's syndrome); GALC [ENSG00000054983] (Krabbe disease); SMPD1, NPC1, NPC2 [ENSG00000166311, ENSG00000141458, ENSG00000119655] (Niemann Pick disease); PRNP [ENSG00000171867] (prion disease); SCN1A [ENSG00000144285] (Dravet syndrome); PINK1, ATPGAP2 [ENSG00000158828] (early-onset Parkinson's disease); ATXN1, ATXN2, ATXN3, PLEKHG4, SPTBN2, CACNA1A, ATXN7, TTBK2, PPP2R2B, KCNC3, PRKCG, ITPR1, TBP, KCND1, FGF14 [ENSG00000124788, ENSG00000204842, ENSG00000066427, ENSG00000196155, ENSG00000173898, ENSG00000141837, ENSG00000163635, ENSG00000128881, ENSG00000156475, ENSG00000131398, ENSG00000126583, ENSG00000150995, ENSG00000112592, ENSG00000102057, ENSG00000102466] (spinocerebellar ataxias); SCN1A, SCN2A, CACNA1A, GRIN2B, GRIN2A, MECP2, FOXG1, SLC6A1, PRRT2, PTEN, KCNQ2, KCNQ3, STARD7, CLRN1 [ENSG00000144285, ENSG00000136531, ENSG00000141837, ENSG00000273079, ENSG00000183454, ENSG00000169057, ENSG00000176165, ENSG00000157103, ENSG00000167371, ENSG00000171862, ENSG00000075043, ENSG00000184156, ENSG00000084090, ENSG00000163646] (genetic epilepsy disorders); ATM [ENSG00000149311] (Ataxia-telangiectasia); GLB1 [ENSG00000170266] (GM1 gangliosidosis); GBA [ENSG00000177628] (Gaucher disease); GM2A [ENSG00000196743] (GM2 gangliosidosis); UBE3A [ENSG00000114062] (Angelman syndrome); SLC2A1 [ENSG00000117394] (glucose transporter deficiency type 1); LAM P2 [ENSG00000005893] (Danon disease); GLA [ENSG00000102393] (Fabry disease); PKD1, PKD2 [ENSG00000008710, ENSG00000118762] (Autosomal dominant polycystic kidney disease); GAA [ENSG00000171298] (Pompe disease); PCSK9, LDLR, APOB, APOE [ENSG00000169174, ENSG00000130164, ENSG00000084674, ENSG00000130203] (Familial hypercholesterolemia); MYOC, OPTN, TBK1, WDR36, CYPIB1 [ENSG00000034971, ENSG00000123240, ENSG00000183735, ENSG00000134987, ENSG00000138061] (Open Angle Glaucoma); IDUA [ENSG00000127415] (Hurler syndrome or Mucopolysaccharidosis 1); IDS [ENSG00000010404] (Hunter syndrome or Mucopolysaccharidosis 2); CLN3 [ENSG00000188603] (Batten disease); DMD [ENSG00000198947] (Duchenne muscular dystrophy); LMNA [ENSG00000160789] (Limb-girdle muscular dystrophy type 1B); DYSF [ENSG00000135636] (Limb-girdle muscular dystrophy type 2B); SGCA [ENSG00000108823] (Limb-girdle muscular dystrophy type 2D); SGCB [ENSG00000163069] (Limb-girdle muscular dystrophy type 2E); SGCG [ENSG00000102683] (Limb-girdle muscular dystrophy type 2C); SGCD [ENSG00000170624] (Limb-girdle muscular dystrophy type 2F); DUX4 [ENSG00000260596] (Facioscapulohumeral muscular dystrophy); F9 [ENSG00000101981] (Hemophilia B); F8 [ENSG00000185010] (Hemophilia A); USHA2A, RPGR, RP2, RHO, PRPF31, USH1F, PRPF3, PRPF6 [ENSG00000156313, ENSG00000102218, ENSG00000163914, ENSG00000105618, ENSG00000150275, ENSG00000117360, ENSG00000101161] (Retinitis pigmentosa); CFTR [ENSG00000001626] (cystic fibrosis); GJB2, GJB6, STRC, DFNA1, WFS1 [ENSG00000165474, ENSG00000121742, ENSG00000242866, ENSG00000131504, ENSG00000109501] (autosomal dominant hearing impairment); POU3F3 [ENSG00000198914] (nonsyndromic hearing loss).

In some embodiments, the replacement domain is codon optimized.

In addition to sequences derived from human genes, Replacement Domains can comprise sequences derived from other organisms in order to alter the stability, translation, processing, or localization of a target RNA. Exemplary replacement domains derived from non-human sources include without limitation sequences that increase protein production such as those derived or isolated from Woodchuck Hepatitis Virus (WHV) Post-transcriptional Regulatory Element (WPRE), triplex from MALAT1, the PRE of Hepatitis B virus (HPRE), and an iron response element of the form CAGYCX (Y=U or A; X=U, C, or A).

### Antisense Domains

In some embodiments of the compositions of the disclosure, a pathogenic RNA molecule is a target RNA. In some embodiments, the target RNA comprises a target sequence that is complementary to an antisense domain of the trans-splicing RNA of the disclosure.

In some embodiments of the compositions and methods of the disclosure, the target sequence comprises or consists of between 5 and 500 nucleotides. In some embodiments, the target sequence comprises or consists of between 50 and 250 nucleotides. In some embodiments, the target sequence comprises or consists of between 5 and 50 nucleotides.

In some embodiments of the compositions and methods of the disclosure, a target sequence is contained within a single contiguous stretch of the target RNA. In some embodiments, the target sequence may consist of comprise of one or more nucleotides that are not spread among a single contiguous stretch of the target RNA.

In some embodiments of the disclosure, a antisense domain of the disclosure binds to a target sequence. In some embodiments of the disclosure, a antisense domain of the disclosure binds to a target RNA.

### Vectors

In some embodiments of the compositions and methods of the disclosure, a vector comprises a trans-splicing nucleic acid of the disclosure. In some embodiments, the vector comprises at least one trans-splicing nucleic acid of the disclosure. In some embodiments, the vector comprises one or more trans-splicing nucleic acid(s) of the disclosure. In some embodiments, the vector comprises two or more trans-splicing nucleic acids of the disclosure.

In some embodiments of the compositions and methods of the disclosure, a vector of the disclosure is a viral vector. In some embodiments, the viral vector comprises a sequence isolated or derived from a retrovirus. In some embodiments, the viral vector comprises a sequence isolated or derived from a lentivirus. In some embodiments, the viral vector comprises a sequence isolated or derived from an adenovirus. In some embodiments, the viral vector comprises a sequence isolated or derived from an adeno-associated virus (AAV). In some embodiments, the viral vector is replication incompetent. In some embodiments, the viral vector is isolated or recombinant. In some embodiments, the viral vector is self-complementary.

In some embodiments of the compositions and methods of the disclosure, the viral vector comprises a sequence isolated or derived from an adeno-associated virus (AAV). In some embodiments, the viral vector comprises an inverted terminal repeat sequence or a capsid sequence that is isolated or derived from an AAV of serotype AAV1, AAV2, AAV3, AAV4, AAV5, AAV6, AAV7, AAV8, AAV9, AAV10, AAV11 or AAV12. In some embodiments, the viral vector is replication incompetent. In some embodiments, the viral vector is isolated or recombinant (rAAV). In some embodiments, the viral vector is self-complementary (scAAV).

In some embodiments of the compositions and methods of the disclosure, a vector of the disclosure is a non-viral vector. In some embodiments, the vector comprises or consists of a nanoparticle, a micelle, a liposome or lipoplex, a polymersome, a polyplex or a dendrimer.

In some embodiments, the vector is an expression vector or recombinant expression system. As used herein, the term“recombinant expression system” refers to a genetic construct for the expression of certain genetic material formed by recombination.

In some embodiments, the liposome, lipoplex, or nanoparticle can further comprise a non-cationic lipid, a PEG conjugated lipid, a sterol, or any combination thereof.

In some embodiments, the the liposome, lipoplex, or nanoparticle further comprises a non-cationic lipid, wherein the non-ionic lipid is selected from the group consisting of distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, I-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine and non-cationic lipids described, for example, in WO2017/099823 or US2018/0028664.

In some embodiments, the liposome, lipoplex, or nanoparticle further comprises a conjugated lipid, wherein the conjugated lipid, wherein the conjugated-lipid is selected from the group consisting of PEG-diacylglycerol (DAG) (such as I-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-O-(2′,3′-di(tetradecanoyloxy)propyl-1-O-(w-methoxy(polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt.

In some embodiments, the liposome, lipoplex, or nanoparticle further comprises cholesterol or a cholesterol derivative.

In some embodiments, the liposome, lipoplex, or nanoparticle further comprises an ionizable lipid, a non-cationic lipid, a conjugated lipid that inhibits aggregation of particles, and a sterol. The amount of the ionizable lipid, the non-cationic lipid, the conjugated lipid that inhibits aggregation of particles, and the sterol can be varied independently. In some embodiments, the lipid nanoparticle comprises an ionizable lipid in an amount from about 20 mol % to about 90 mol % of the total lipid present in the particle, a non-cationic lipid in an amount from about 5 mol % to about 30 mol % of the total lipid present in the particle, a conjugated lipid that inhibits aggregation of particles in an amount from about 0.5 mol % to about 20 mol % of the total lipid present in the particle, and a sterol in an amount from about 20 mol % to about 50 mol % of the total lipid present in the particle.

The ratio of total lipid to DNA vector can be varied as desired. For example, the total lipid to DNA vector (mass or weight) ratio can be from about 10:1 to about 30:1.

In some embodiments of the compositions and methods of the disclosure, an expression vector, viral vector or non-viral vector provided herein, includes without limitation, an expression control element. An“expression control element” as used herein refers to any sequence that regulates the expression of a coding sequence, such as a gene. Exemplary expression control elements include but are not limited to promoters, enhancers, microRNAs, post-transcriptional regulatory elements, polyadenylation signal sequences, and introns. Expression control elements may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. In some embodiments, expression control by a promoter is tissue-specific. Non-limiting exemplary promoters include CMV, CBA, CAG, Cbh, EF-1a, PGK, UBC, GUSB, UCOE, hAAT, TBG, Desmin, MCK, C5-12, NSE, Synapsin, PDGF, MecP2, CaMKII, mGluR2, NFL, NFH, n132, PPE, ENK, EAAT2, GFAP, MBP, and U6 promoters. An“enhancer” is a region of DNA that can be bound by activating proteins to increase the likelihood or frequency of transcription. Non-limiting exemplary enhancers and posttranscriptional regulatory elements include the CMV enhancer and WPRE.

### Nucleic Acids

Provided herein are the nucleic acid sequences encoding the trans-splicing nucleic acids disclosed herein for use in gene transfer and expression techniques described herein. It should be understood, although not always explicitly stated that the sequences provided herein can be used to provide the expression product as well as substantially identical sequences that produce a protein that has the same biological properties. These“biologically equivalent” or“biologically active” or“equivalent” polypeptides are encoded by equivalent polynucleotides as described herein. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical nucleic acid sequence to the reference nucleic acid sequence when compared using sequence identity methods run under default conditions. Specific sequences are provided as examples of particular embodiments. Additionally, an equivalent polynucleotide is one that hybridizes under stringent conditions to the reference polynucleotide or its complement.

The nucleic acid sequences (e.g., polynucleotide sequences) disclosed herein may be codon-optimized which is a technique well known in the art. Codon optimization refers to the fact that different cells differ in their usage of particular codons. This codon bias corresponds to a bias in the relative abundance of particular tRNAs in the cell type. By altering the codons in the sequence to match with the relative abundance of corresponding tRNAs, it is possible to increase expression. It is also possible to decrease expression by deliberately choosing codons for which the corresponding tRNAs are known to be rare in a particular cell type. Codon usage tables are known in the art for mammalian cells, as well as for a variety of other organisms. Based on the genetic code, nucleic acid sequences coding for various replacement domains can be generated. In some embodiments, such a sequence is optimized for expression in a host or target cell, such as a host cell used to express the trans-splicing RNA containing a replacement domain in which the disclosed methods are practiced (such as in a mammalian cell, e.g., a human cell). Codon preferences and codon usage tables for a particular species can be used to engineer isolated nucleic acid molecules encoding a replacement domain (such as one encoding a protein having at least 80%, at least 85%, at least 90%, at least 92%, at least 95%, at least 96%, at least 97%, at least 98%, at least 99%, or 100% sequence identity to its corresponding wild-type protein) that takes advantage of the codon usage preferences of that particular species. For example, the replacement domains disclosed herein can be designed to have codons that are preferentially used by a particular organism of interest. In one example, a replacement domain nucleic acid sequence is optimized for expression in human cells, such as one having at least 70%, at least 80%, at least 85%, at least 90%, at least 92%, at least 95%, at least 98%, or at least 99% sequence identity to its corresponding wild-type or originating nucleic acid sequence. In some embodiments, an isolated trans-splicing nucleic acid molecule encoding at least one replacement domain (which can be part of a vector) includes at least one replacement domain coding sequence that is codon optimized for expression in a eukaryotic cell, or at least one replacement domain coding sequence codon optimized for expression in a human cell. In one embodiment, such a codon optimized replacement domain coding sequence has at least 80%, at least 85%, at least 90%, at least 92%, at least 95%, at least 96%, at least 97%, at least 98%, at least 99%, or 100% sequence identity to its corresponding wild-type or originating sequence. In another embodiment, a eukaryotic cell codon optimized nucleic acid sequence encodes a replacement domain having at least 85%, at least 90%, at least 92%, at least 95%, at least 96%, at least 97%, at least 98%, at least 99%, or 100% sequence identity to its corresponding wild-type or originating protein. In another embodiment, a variety of clones containing functionally equivalent nucleic acids may be routinely generated, such as nucleic acids which differ in sequence but which encode the same replacement domain protein sequence. Silent mutations in the coding sequence result from the degeneracy (i.e., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC; aspartic acid can be encoded by GAT or GAC; cysteine can be encoded by TGT or TGC; alanine can be encoded by GCT, GCC, GCA, or GCG; glutamine can be encoded by CAA or CAG; tyrosine can be encoded by TAT or TAC; and isoleucine can be encoded by ATT, ATC, or ATA. Tables showing the standard genetic code can be found in various sources (see, for example, Stryer, 1988, Biochemistry, 3.sup.rd Edition, W.H.5 Freeman and Co., NY).

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PC reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC;

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

### Methods of Use

The disclosure provides a method of modifying the composition of a target RNA of the disclosure primarily in a specific tissue comprising contacting the composition and the target RNA under conditions suitable for trans-splicing among the composition and the target RNA.

The disclosure provides a method of modifying the composition of a target RNA of the disclosure primarily in a specific tissue comprising contacting the composition and a cell comprising the target RNA under conditions suitable for trans-splicing among the composition and the target RNA.

The disclosure provides a method of modifying the composition of a protein encoded by a target RNA of the disclosure primarily in a specific tissue comprising contacting the composition and the target RNA under conditions suitable for trans-splicing among the composition and the target RNA.

The disclosure provides a method of modifying the composition of a protein encoded by a target RNA of the disclosure primarily in a specific tissue comprising contacting the composition and a cell comprising the target RNA under conditions suitable for trans-splicing among the composition and the target RNA.

The disclosure provides a method of modifying the composition of a target RNA of the disclosure primarily in liver tissue comprising contacting the composition and a liver cell comprising the target RNA under conditions suitable for trans-splicing among the composition and the target RNA.

The disclosure provides a method of modifying the composition of a protein encoded by a target RNA of the disclosure primarily in liver tissue comprising contacting the composition and a liver cell comprising the target RNA under conditions suitable for trans-splicing among the composition and the target RNA.

The disclosure provides a method of modifying the composition of a target RNA of the disclosure primarily in muscle tissue comprising contacting the composition and a muscle cell comprising the target RNA under conditions suitable for trans-splicing among the composition and the target RNA.

The disclosure provides a method of modifying the composition of a protein encoded by a target RNA of the disclosure primarily in muscle tissue comprising contacting the composition and a muscle cell comprising the target RNA under conditions suitable for trans-splicing among the composition and the target RNA.

The disclosure provides a method of modifying the composition of a target RNA of the disclosure primarily in brain tissue comprising contacting the composition and a brain cell comprising the target RNA under conditions suitable for trans-splicing among the composition and the target RNA.

The disclosure provides a method of modifying the composition of a protein encoded by a target RNA of the disclosure primarily in brain tissue comprising contacting the composition and a brain cell comprising the target RNA under conditions suitable for trans-splicing among the composition and the target RNA.

The disclosure provides a method of modifying the composition of a target RNA of the disclosure primarily in heart tissue comprising contacting the composition and a heart cell comprising the target RNA under conditions suitable for trans-splicing among the composition and the target RNA.

The disclosure provides a method of modifying the composition of a protein encoded by a target RNA of the disclosure primarily in heart tissue comprising contacting the composition and a heart cell comprising the target RNA under conditions suitable for trans-splicing among the composition and the target RNA.

The disclosure provides a method of modifying the composition of a target RNA of the disclosure primarily in retina tissue comprising contacting the composition and a retina cell comprising the target RNA under conditions suitable for trans-splicing among the composition and the target RNA.

The disclosure provides a method of modifying the composition of a protein encoded by a target RNA of the disclosure primarily in retina tissue comprising contacting the composition and a retina cell comprising the target RNA under conditions suitable for trans-splicing among the composition and the target RNA.

The disclosure provides a method of modifying the composition of a target RNA of the disclosure primarily in a specific tissue comprising contacting the composition and a cell comprising the target RNA under conditions suitable for trans-splicing among the composition and the target RNA. In some embodiments, the cell is in vivo, in vitro, ex vivo or in situ. In some embodiments, the composition comprises a vector comprising composition comprising a guide RNA of the disclosure and a fusion protein of the disclosure. In some embodiments, the vector is an AAV.

The disclosure provides a method of modifying the composition of a protein encoded by a target RNA of the disclosure primarily in a specific tissue comprising contacting the composition and a cell comprising the target RNA under conditions suitable for trans-splicing among the composition and the target RNA. In some embodiments, the cell is in vivo, in vitro, ex vivo or in situ. In some embodiments, the composition comprises a vector comprising composition comprising a guide RNA of the disclosure and a fusion protein of the disclosure. In some embodiments, the vector is an AAV.

The disclosure provides a method of treating a disease or disorder comprising administering to a subject a therapeutically effective amount of a composition of the disclosure.

The disclosure provides a method of treating a disease or disorder comprising administering to a subject a therapeutically effective amount of a composition of the disclosure, wherein the composition comprises a vector comprising composition comprising a guide RNA of the disclosure and a fusion protein of the disclosure and wherein the composition modifies a level of expression of an RNA molecule of the disclosure or a protein encoded by the RNA molecule.

The disclosure provides a method of treating a disease or disorder comprising administering to a subject a therapeutically effective amount of a composition of the disclosure, wherein the composition comprises a vector comprising composition comprising a guide RNA of the disclosure and a fusion protein of the disclosure and wherein the composition modifies an activity of a protein encoded by an RNA molecule.

In some embodiments of the compositions and methods of the disclosure, a disease or disorder of the disclosure includes, but is not limited to, a genetic disease or disorder. In some embodiments, the genetic disease or disorder is a single-gene disease or disorder. In some embodiments, the single-gene disease or disorder is an autosomal dominant disease or disorder, an autosomal recessive disease or disorder, an X-chromosome linked (X-linked) disease or disorder, an X-linked dominant disease or disorder, an X-linked recessive disease or disorder, a Y-linked disease or disorder or a mitochondrial disease or disorder. In some embodiments, the singe-gene disease or disorder is, but not limited to, common variable immune deficiency, Adenosine deaminase deficiency, X-linked severe combined immunodeficiency, Beta-thassalemia, alpha-thassalemia, myelodysplastic syndrome, Amyotrophic lateral sclerosis, Frontotemporal dementia with parkinsonism, Usher's syndrome, Krabbe disease, Niemann Pick disease, prion disease, Dravet syndrome, early-onset Parkinson's disease, spinocerebellar ataxias, genetic epilepsy disorders, Ataxia-telangiectasia, GM1 gangliosidosis, Gaucher disease, GM2 gangliosidosis, Angelman syndrome, glucose transporter deficiency type 1, Danon disease, Fabry disease, Autosomal dominant polycystic kidney disease, Pompe disease, Familial hypercholesterolemia, Open Angle Glaucoma, Hurler syndrome or Mucopolysaccharidosis 1, Hunter syndrome or Mucopolysaccharidosis 2, Batten disease, Duchenne muscular dystrophy, Limb-girdle muscular dystrophy type 1B, Limb-girdle muscular dystrophy type 2B, Limb-girdle muscular dystrophy type 2D, Limb-girdle muscular dystrophy type 2E, Limb-girdle muscular dystrophy type 2C, Limb-girdle muscular dystrophy type 2F, Facioscapulohumeral muscular dystrophy, Hemophilia B, Hemophilia A, Retinitis pigmentosa, cystic fibrosis, autosomal dominant hearing impairment, and non-syndromic hearing loss. In some embodiments, the genetic disease or disorder is a multiple-gene disease or disorder. In some embodiments, the genetic disease or disorder is a multiple-gene disease or disorder. In some embodiments, the single-gene disease or disorder is an autosomal dominant disease or disorder including, but not limited to, Huntington's disease, neurofibromatosis type 1, neurofibromatosis type 2, Marfan syndrome, hereditary nonpolyposis colorectal cancer, hereditary multiple exostoses, Von Willebrand disease, and acute intermittent porphyria. In some embodiments, the single-gene disease or disorder is an autosomal recessive disease or disorder including, but not limited to, Albinism, Medium-chain acyl-CoA dehydrogenase deficiency, cystic fibrosis, sickle-cell disease, Tay-Sachs disease, Niemann-Pick disease, spinal muscular atrophy, and Roberts syndrome. In some embodiments, the single-gene disease or disorder is X-linked disease or disorder including, but not limited to, muscular dystrophy, Duchenne muscular dystrophy, Hemophilia, Adrenoleukodystrophy (ALD), Rett syndrome, and Hemophilia A. In some embodiments, the single-gene disease or disorder is a mitochondrial disorder including, but not limited to, Leber's hereditary optic neuropathy.

In some embodiments of the compositions and methods of the disclosure, a disease or disorder of the disclosure includes, but is not limited to, an immune disease or disorder. In some embodiments, the immune disease or disorder is an immunodeficiency disease or disorder including, but not limited to, B-cell deficiency, T-cell deficiency, neutropenia, asplenia, complement deficiency, acquired immunodeficiency syndrome (AIDS) and immunodeficiency due to medical intervention (immunosuppression as an intended or adverse effect of a medical therapy). In some embodiments, the immune disease or disorder is an autoimmune disease or disorder including, but not limited to, Achalasia, Addison's disease, Adult Still's disease, Agammaglobulinemia, Alopecia areata, Amyloidosis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, Vogt-Koyanagi-Harada Disease, or Wegener's granulomatosis.

In some embodiments of the compositions and methods of the disclosure, a disease or disorder of the disclosure includes, but is not limited to, an inflammatory disease or disorder.

In some embodiments of the compositions and methods of the disclosure, a disease or disorder of the disclosure includes, but is not limited to, a metabolic disease or disorder.

In some embodiments of the compositions and methods of the disclosure, a disease or disorder of the disclosure includes, but is not limited to, a degenerative or a progressive disease or disorder. In some embodiments, the degenerative or a progressive disease or disorder includes, but is not limited to, amyotrophic lateral sclerosis (ALS), Huntington's disease, Alzheimer's disease, and aging.

In some embodiments of the compositions and methods of the disclosure, a disease or disorder of the disclosure includes, but is not limited to, an infectious disease or disorder.

In some embodiments of the compositions and methods of the disclosure, a disease or disorder of the disclosure includes, but is not limited to, a pediatric or a developmental disease or disorder.

In some embodiments of the compositions and methods of the disclosure, a disease or disorder of the disclosure includes, but is not limited to, a cardiovascular disease or disorder.

In some embodiments of the compositions and methods of the disclosure, a disease or disorder of the disclosure includes, but is not limited to, a proliferative disease or disorder. In some embodiments, the proliferative disease or disorder is a cancer. In some embodiments, the cancer includes, but is not limited to, Acute Lymphoblastic Leukemia (ALL), Acute Myeloid Leukemia (AML), Adrenocortical Carcinoma, AIDS-Related Cancers, Kaposi Sarcoma (Soft Tissue Sarcoma), AIDS-Related Lymphoma (Lymphoma), Primary CNS Lymphoma (Lymphoma), Anal Cancer, Appendix Cancer, Gastrointestinal Carcinoid Tumors, Astrocytomas, Atypical Teratoid/Rhabdoid Tumor, Central Nervous System (Brain Cancer), Basal Cell Carcinoma, Bile Duct Cancer, Bladder Cancer, Bone Cancer, Ewing Sarcoma, Osteosarcoma, Malignant Fibrous Histiocytoma, Brain Tumors, Breast Cancer, Burkitt Lymphoma, Carcinoid Tumor, Carcinoma, Cardiac (Heart) Tumors, Embryonal Tumors, Germ Cell Tumor, Primary CNS Lymphoma, Cervical Cancer, Cholangiocarcinoma, Chordoma, Chronic Lymphocytic Leukemia (CLL), Chronic Myelogenous Leukemia (CML), Chronic Myeloproliferative Neoplasms, Colorectal Cancer, Craniopharyngioma, Cutaneous T-Cell Lymphoma, Ductal Carcinoma In Situ, Embryonal Tumors, Endometrial Cancer (Uterine Cancer), Ependymoma, Esophageal Cancer, Esthesioneuroblastoma (Head and Neck Cancer), Ewing Sarcoma (Bone Cancer), Extracranial Germ Cell Tumor, Extragonadal Germ Cell Tumor, Eye Cancer, Childhood Intraocular Melanoma, Intraocular Melanoma, Retinoblastoma, Fallopian Tube Cancer, Fibrous Histiocytoma of Bone, Malignant, and Osteosarcoma, Gallbladder Cancer, Gastric (Stomach) Cancer, Gastrointestinal Carcinoid Tumor, Gastrointestinal Stromal Tumors (GIST) (Soft Tissue Sarcoma), Childhood Gastrointestinal Stromal Tumors, Germ Cell Tumors, Childhood Extracranial Germ Cell Tumors, Extragonadal Germ Cell Tumors, Ovarian Germ Cell Tumors, Testicular Cancer, Gestational Trophoblastic Disease, Hairy Cell Leukemia, Head and Neck Cancer, Heart Tumors, Hepatocellular (Liver) Cancer, Histiocytosis, Hodgkin Lymphoma, Hypopharyngeal Cancer (Head and Neck Cancer), Intraocular Melanoma, Islet Cell Tumors, Pancreatic Neuroendocrine Tumors, Kaposi Sarcoma (Soft Tissue Sarcoma), Kidney (Renal Cell) Cancer, Langerhans Cell Histiocytosis, Laryngeal Cancer (Head and Neck Cancer), Leukemia, Lip and Oral Cavity Cancer (Head and Neck Cancer), Liver Cancer, Lung Cancer (Non-Small Cell and Small Cell), Childhood Lung Cancer, Lymphoma, Male Breast Cancer, Malignant Fibrous Histiocytoma of Bone and Osteosarcoma, Melanoma, Merkel Cell Carcinoma (Skin Cancer), Mesothelioma, Metastatic Squamous Neck Cancer with Occult Primary (Head and Neck Cancer), Midline Tract Carcinoma With NUT Gene Changes, Mouth Cancer (Head and Neck Cancer), Multiple Endocrine Neoplasia Syndromes, Multiple Myeloma/Plasma Cell Neoplasms, Mycosis Fungoides (Lymphoma), Myelodysplastic Syndromes, Myelodysplastic/Myeloproliferative Neoplasms, Nasal Cavity and Paranasal Sinus Cancer (Head and Neck Cancer), Nasopharyngeal Cancer (Head and Neck Cancer), Neuroblastoma, Non-Hodgkin Lymphoma, Non-Small Cell Lung Cancer, Oral Cancer, Lip and Oral Cavity Cancer and Oropharyngeal Cancer, Osteosarcoma and Malignant Fibrous Histiocytoma of Bone, Ovarian Cancer, Pancreatic Cancer, Pancreatic Neuroendocrine Tumors (Islet Cell Tumors), Papillomatosis, Paraganglioma, Parathyroid Cancer, Penile Cancer, Pharyngeal Cancer (Head and Neck Cancer), Pheochromocytoma, Plasma Cell Neoplasm/Multiple Myeloma, Pleuropulmonary Blastoma, Pregnancy and Breast Cancer, Primary Central Nervous System (CNS) Lymphoma, Primary Peritoneal Cancer, Prostate Cancer, Rectal Cancer, Recurrent Cancer, Renal Cell (Kidney) Cancer, Retinoblastoma, Rhabdomyosarcoma, Childhood (Soft Tissue Sarcoma), Salivary Gland Cancer (Head and Neck Cancer), Sarcoma, Childhood Rhabdomyosarcoma (Soft Tissue Sarcoma), Childhood Vascular Tumors (Soft Tissue Sarcoma), Ewing Sarcoma (Bone Cancer), Kaposi Sarcoma (Soft Tissue Sarcoma), Osteosarcoma (Bone Cancer), Uterine Sarcoma, S√©zary Syndrome, Lymphoma, Skin Cancer, Small Cell Lung Cancer, Small Intestine Cancer, Soft Tissue Sarcoma, Squamous Cell Carcinoma of the Skin, Squamous Neck Cancer, Stomach (Gastric) Cancer, T-Cell Lymphoma, Testicular Cancer, Throat Cancer (Head and Neck Cancer), Nasopharyngeal Cancer, Oropharyngeal Cancer, Hypopharyngeal Cancer, Thymoma and Thymic Carcinoma, Thyroid Cancer, Transitional Cell Cancer of the Renal Pelvis and Ureter, Renal Cell Cancer, Urethral Cancer, Uterine Sarcoma, Vaginal Cancer, Vascular Tumors (Soft Tissue Sarcoma), Vulvar Cancer, Wilms Tumor and Other Childhood Kidney Tumors.

In some embodiments of the compositions and methods of the disclosure, a disease or disorder of the disclosure includes, but is not limited to, a proliferative disease or disorder. In some embodiments, the proliferative disease or disorder is a cancer. In some embodiments, the cancer involves the present of a gene fusion that produces a chimeric RNA with sequences derived from two genes due to a deletion or translocation of DNA. Gene fusions pairs include but are not limited to: MAN2A1 and FER, DNAJB1 and PRKACA, BCR-ABL1, TMPRSS2 and ERG, EWSR1 and FLI1, PML and RARA, EML4 and ALK, KIAA1549 and BRAF, CCDC6 and RET, SS18 and SSX1, RUNX1 and RUNX1T1, PAX3 and FOXO1, NCOA4 and RET, ETV6 and RUNX1, FUS and DDIT3, SS18 and SSX2, NPM1 and ALK, KMT2A and AFF1, TCF3 and PBX1, STIL and TALI, COL1A1 and PDGFB, CRTC1 and MAML2, NAB2 and STATE, EWSR1 and ATF1, ETV6 and NTRK3, EWSR1 and ERG, EWSR1 and WT1, DNAJB1 and PRKACA, PAX7 and FOXO1, FUS and CREB3L2, CBFA2T3 and GLIS2, PAX8 and PPARG, KMT2A and MLLT1, EWSR1 and NR4A3, KMT2A and MLLT3, ASPSCR1 and TFE3, HMGA2 and LPP, JAZF1 and SUZ12, KIF5B and RET, FUS and ERG, SLC45A3 and ERG, NUP214 and ABL1, SET and NUP214, CD74 and ROS1, ETV6 and ABL1, TPM3 and NTRK1, PRKAR1A and RET, EWSR1 and CREB1, KMT2A and AFDN, EWSR1 and DDIT3, CLTC and ALK, ETV6 and PDGFRB, TPM3 and ALK, KMT2A and MLLT10, TMPRSS2 and ETV1, BRD4 and NUTM1, NUP98 and KDM5A, RANBP2 and ALK, CTNNB1 and PLAG1, KMT2A and ELL, TAF15 and NR4A3, FGFR3 and TACC3, PCM1 and JAK2, YWHAE and NUTM2B, STRN and ALK, CRTC3 and MAML2, CDH11 and USP6, CDKN2D and WDFY2, CIC and DUX4, SLC34A2 and ROS1, ATIC and ALK, CD74 and NRG1, MYB and NFIB, PRCC and TFE3, KIF5B and ALK, TMPRSS2 and ETV4, KMT2A and SEPT9, EWSR1 and POU5F1, FGFR1 and PLAG1, MN1 and ETV6, TBL1XR1 and TP63, KMT2A and EPS15, SLC45A3 and ELK4, DHH and RHEBL1, HEY1 and NCOA2, EZR and ROS1, GOPC and ROS1, HMGA2 and WIF1, KMT2A and CREBBP, SS18 and SSX4B, FAM131B and BRAF, EWSR1 and FEV, EWSR1 and PBX1, TPM4 and ALK, SND1 and BRAF, ACTB and GLI1, KMT2A and KNL1, KMT2A and SEPT6, SDC4 and ROS1, TFG and ALK, HNRNPA2B1 and ETV1, PTPRK and RSPO3, JAZF1 and PHF1, HMGA2 and RAD51B, KMT2A and MLLT11, TPR and NTRK1, AKAP9 and BRAF, FUS and CREB3L1, ETV6 and JAK2, HMGA2 and NFIB, KMT2A and AFF3, CHCHD7 and PLAG1, VTI1A and TCF7L2, LIFR and PLAG1, EWSR1 and ETV1, SRGAP3 and RAF1, KMT2A and AFF4, MEAF6 and PHF1, PAX3 and NCOA1, HAS2 and PLAG1, EWSR1 and NFATC2, HIP1 and ALK, GOLGA5 and RET, BCR and JAK2, EWSR1 and ETV4, DCTN1 and ALK, MBTD1 and CXorf67, NDRG1 and ERG, CARS and ALK, SFPQ and TFE3, KMT2A and ARHGAP26, KMT2A and EP300, KMT2A and TET1, PAX5 and JAK2, PPFIBP1 and ALK, YWHAE and NUTM2A, LRIG3 and ROS1, TFG and NTRK1, TPM3 and ROS1, SLC45A3 and ETV1, ERC1 and RET, SEC16A and NOTCH1, KTN1 and RET, SEC31A and JAK2, TCEA1 and PLAG1, QKI and NTRK2, RNF130 and BRAF, EIF3E and RSPO2, EWSR1 and ZNF444, LMNA and NTRK1, PPFIBP1 and ROS1, PWWP2A and ROS1, EWSR1 and YY1, FUS and ATF1, PAX3 and NCOA2, ZC3H7B and BOOR, BRD3 and NUTM1, CANT1 and ETV4, CIC and FOXO4, COL1A1 and USP6, EWSR1 and ZNF384, KMT2A and ABI1, KMT2A and ACTN4, KMT2A and CEP170B, KMT2A and FOXO3, KMT2A and GAS7, KMT2A and MLLT6, KMT2A and SEPT2, KMT2A and SEPT5, MSN and ALK, VCL and ALK, EZR and ERBB4, RELCH and RET, SLC3A2 and NRG1, TRIM24 and BRAF, KLC1 and ALK, ARID1A and MAST2, GPBP1L1 and MAST2, NFIX and MAST1, NOTCH1 and GABBR2, TADA2A and MAST1, ZNF700 and MAST1, TRIM24 and RET, TRIM33 and RET, SSBP2 and JAK2, KMT2A and EEFSEC, CLCN6 and BRAF, GNAI1 and BRAF, MKRN1 and BRAF, NACC2 and NTRK2, FGFR1 and TACC1, TRIM27 and RET, HMGA2 and FHIT, HOOK3 and RET, PCM1 and RET, CEP89 and BRAF, CLIP1 and ROS1, ERC1 and ROS1, HLA and A and ROS1, LSM14A and BRAF, MYO5A and ROS1, SHTN1 and ROS1, TP53 and NTRK1, TPM3 and ROS1, ZCCHC8 and ROS1, FGFR3 and BAIAP2L1, KLK2 and ETV1, ACSL3 and ETV1, NUP107 and LGR5, HMGA2 and CCNB1IP1, HMGA2 and COX6C, GATM and BRAF, HACL1 and RAF1, HERPUD1 and BRAF, ZSCAN30 and BRAF, SLC45A3 and BRAF, HMGA2 and LHFPL6, COL1A2 and PLAG1, ESRP1 and RAF1, IRF2BP2 and CDX1, TFG and NR4A3, CLTC and TFE3, EWSR1 and MYB, NONO and TFE3, FCHSD1 and BRAF, HMGA2 and EBF1, ACBD6 and RRP15, AGPAT5 and MCPH1, AGTRAP and BRAF, ARFIP1 and FHDC1, ATG4C and FBXO38, BBS9 and PKD1 L1, CENPK and KMT2A, CNBP and USP6, DDX5 and ETV4, EIF3K and CYP39A1, EPC1 and PHF1, ERO1A and FERMT2, ETV6 and ITPR2, EWSR1 and NFATC1, EWSR1 and PATZ1, EWSR1 and SMARCA5, EWSR1 and SP3, FBXL18 and RNF216, FGFR1 and ZNF703, FN1 and ALK, FUS and FEV, GMDS and PDE8B, HMGA2 and ALDH2, IL6R and ATP8B2, INTS4 and GAB2, JPT1 and USH1G, KLK2 and ETV4, KMT2A and ABI2, KMT2A and ARHGEF12, KMT2A and BTBD18, KMT2A and CASP8AP2, KMT2A and CBL, KMT2A and CIP2A, KMT2A and CT45A2, KMT2A and DAB2IP, KMT2A and FOXO4, KMT2A and FRYL, KMT2A and GMPS, KMT2A and GPHN, KMT2A and LASP1, KMT2A and LPP, KMT2A and MAPRE1, KMT2A and MYO1F, KMT2A and NCKIPSD, KMT2A and NRIP3, KMT2A and PDS5A, KMT2A and PICALM, KMT2A and PRRC1, KMT2A and SARNP, KMT2A and SH3GL1, KMT2A and SORBS2, KMT2A and TOP3A, KMT2A and ZFYVE19, MBOAT2 and PRKCE, MIA2 and GEMIN2, NF1 and ASIC2, NFIA and EHF, NTN1 and ACLY, OMD and USP6, PLA2R1 and RBMS1, PLXND1 and TMCC1, RAF1 and DAZL, RBM14 and PACS1, RGS22 and SYCP1, SEC31A and ALK, SEPT8 and AFF4, SLC22A1 and CUTA, SLC26A6 and PRKAR2A, SLC45A3 and ETV5, SQSTM1 and ALK, SS18L1 and SSX1, SSH2 and SUZ12, SUSD1 and PTBP3, TCF12 and NR4A3, TECTA and TBCEL, THRAP3 and USP6, TMPRSS2 and ETV5, TPR and ALK, UBE2L3 and KRAS, WDCP and ALK, SS18 and USP6

In some embodiments of the methods of the disclosure, a subject of the disclosure has been diagnosed with the disease or disorder. In some embodiments, the subject of the disclosure presents at least one sign or symptom of the disease or disorder. In some embodiments, the subject has a biomarker predictive of a risk of developing the disease or disorder. In some embodiments, the biomarker is a genetic mutation.

In some embodiments of the methods of the disclosure, a subject of the disclosure is female. In some embodiments of the methods of the disclosure, a subject of the disclosure is male. In some embodiments, a subject of the disclosure has two XX or XY chromosomes. In some embodiments, a subject of the disclosure has two XX or XY chromosomes and a third chromosome, either an X or a Y.

In some embodiments of the methods of the disclosure, a subject of the disclosure is a neonate, an infant, a child, an adult, a senior adult, or an elderly adult. In some embodiments of the methods of the disclosure, a subject of the disclosure is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 days old. In some embodiments of the methods of the disclosure, a subject of the disclosure is at least 1, 2, 3, 4, 5, 6, 7, 8, 9, 10, 11 or 12 months old. In some embodiments of the methods of the disclosure, a subject of the disclosure is at least 1, 2, 3, 4, 5, 6, 7, 8, 9, 10, 15, 20, 25, 30, 35, 40, 45, 50, 55, 60, 65, 70, 75, 80, 85, 90, 95, 100 or any number of years or partial years in between of age.

In some embodiments of the methods of the disclosure, a subject of the disclosure is a mammal. In some embodiments, a subject of the disclosure is a non-human mammal.

In some embodiments of the methods of the disclosure, a subject of the disclosure is a human.

In some embodiments of the methods of the disclosure, a therapeutically effective amount comprises a single dose of a composition of the disclosure. In some embodiments, a therapeutically effective amount comprises a therapeutically effective amount comprises at least one dose of a composition of the disclosure. In some embodiments, a therapeutically effective amount comprises a therapeutically effective amount comprises one or more dose(s) of a composition of the disclosure.

In some embodiments of the methods of the disclosure, a therapeutically effective amount eliminates a sign or symptom of the disease or disorder. In some embodiments, a therapeutically effective amount reduces a severity of a sign or symptom of the disease or disorder.

In some embodiments of the methods of the disclosure, a therapeutically effective amount eliminates the disease or disorder.

In some embodiments of the methods of the disclosure, a therapeutically effective amount prevents an onset of a disease or disorder. In some embodiments, a therapeutically effective amount delays the onset of a disease or disorder. In some embodiments, a therapeutically effective amount reduces the severity of a sign or symptom of the disease or disorder. In some embodiments, a therapeutically effective amount improves a prognosis for the subject.

In some embodiments of the methods of the disclosure, a composition of the disclosure is administered to the subject systemically. In some embodiments, the composition of the disclosure is administered to the subject by an intravenous route. In some embodiments, the composition of the disclosure is administered to the subject by an injection or an infusion.

In some embodiments of the methods of the disclosure, a composition of the disclosure is administered to the subject locally. In some embodiments, the composition of the disclosure is administered to the subject by an intraosseous, intraocular, intracerebrospinal or intraspinal route. In some embodiments, the composition of the disclosure is administered directly to the cerebral spinal fluid of the central nervous system. In some embodiments, the composition of the disclosure is administered directly to a tissue or fluid of the eye and does not have bioavailability outside of ocular structures. In some embodiments, the composition of the disclosure is administered to the subject by an injection or an infusion.

In some embodiments, the compositions comprising the trans-splicing RNAs disclosed herein are formulated as pharmaceutical compositions. Briefly, pharmaceutical compositions for use as disclosed herein may comprise a fusion protein(s) or a polynucleotide encoding the fusion protein(s), optionally comprised in an AAV, which is optionally also immune orthogonal, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the disclosure may be formulated for oral, intravenous, topical, enteral, intraocular, and/or parenteral administration. In certain embodiments, the compositions of the present disclosure are formulated for intravenous administration.

### EXAMPLE EMBODIMENTS

Embodiment 1: A trans-splicing nucleic acid, comprising: (a) one or more replacement domains that encode a therapeutic sequence operably linked to; (b) one or more intronic domains that promote RNA splicing of the replacement domain primarily in a specific tissue or cell type; and (c) one or more antisense domains that promote binding to a target RNA molecule.

Embodiment 2: The composition of embodiment 1, wherein the intronic domains promote trans-splicing primarily in brain tissue and are derived or isolated from genes selected from the group consisting of: UBB, GFAP, STMN1, RPL13A, UCHL1, TUBB2B, ALDOA, LRRC75A-AS1, DYNLRB1, TPI1, PKM, NDRG2, MT3, RTN3, SKP1, RTN1, NRGN, DBNDD2, BSCL2, ALDOC, S100B, GNG3, FXYD6, PRDX1, HSP90AA1, SRP14, ATF4, NDUFB8, CAPZB, ATF5, ADD1, ERGIC3, GUK1, DCTN1, GSN, MORF4L1, GNAI2, SPP1, CRIP2, RNASE1, EIF4G2, PAQR6, DCTN3, CALB2, COMMD6, MEST, STXBP1, CLDND1, SH3GLB2, NREP, ABCA2, SUN2, UBXN6, DBI, CDK2AP1, NFIX, SF1, HNRNPF, GPM6B, SQSTM1, MOBP, ANAPC11, POLD2, SAMD4B, IST1, TPD52, SHD, STK25, ABR, PQBP1, PHB, SEPT9, S100A13, APBB1, RANBP1, ARHGEF2, DLGAP4, GNAO1, SEZ6L2, FDFT1, CBX1, EIF4G1, FAM213A, MAPK8IP1, PSME1, CCAR2, TKT, SYN1, OGDH, OCIAD1, SLC3A2, SLC1A5, PTTG1, DNM1, MAP6, KDM1A, PTN, FN1, CSNK1E, TMEM9, MATR3, AHCYL1, ATP6V0A1, CDIP1, CYFIP2, SLIRP, SCG5, TPX2, DMTN, SSBP1, PFKL, KIAA0930, CAMTA1, PRC1, CHTF8, TMPRSS3, BIN1, CCT8, AMZ2, TM7SF2, SEC11A, CLPTM1, AMPD2, DHX9, GAB2, CLTC, PSME3, RPP21, DYNC1I2, CDC20, NOSIP, ATP13A2, AIF1L, SURF4, PTPRN2, STK24, SDR39U1, HMOX2, UBAP2L, NRCAM, CCDC3, GPT2, TN P02, NRXN2, PPP1R7, CAMK2A, UBE2L6, DBNL, ADAR, RNF220, MCM2, DTNA, PDIA6, PSMG2, URM1, SNX17, SV2A, SPG21, REEP2, HSD17B4, GSTO1, TRAPPC3, ZNF385A, SHISA5, FAM107B, MID1IP1, SERPINE2, CDK5RAP3, COMMD4, ANAPC15, CABP1, PTPRA, NPTN, RERE, VPS29, SPTBN2, MT1G, DTX3, GOLGA7, TPD52L2, EPB41L3, SPIN1, FARP1, NUP62, CNOT1, TMEM108, DUT, MAPRE2, PSD, NEBL, UBE2L3, KIF5C, TCF19, WDR54, CPSF7, GIT1, L1CAM, ITPR3, RHPN2, HMGA1, KCNAB2, HID1, SHF, CD99L2, C19ORF25, PAF1, B4GALT2, PTGES2, CAPG, SEMA4D, TTYH2, EXOC7, CNDP1, TACC2, BACE1, GABBR1, LMNB1, SLAIN1, GLG1, APBA2, CADM3, PC, PHLDB1, HHATL, SOGA1, CNTNAP2, RFC2, MAPK8IP3, CD200, REC8, TACC3, SYT13, SRP68, KLC4, CORO1C, ATPAF1, NARF, AP3B2, NUP93, FAM102A, SCN1B, NDUFS2, CBFA2T2, PSMA5, EPB41, and BCAR1.

Embodiment 3: The composition of embodiment 2, wherein the intronic domains promote trans-splicing primarily in brain tissue and are selected from the group consisting of SEQ ID NO 1-SEQ ID NO 492.

Embodiment 4: The composition of embodiments 2 or 3, wherein the replacement domain is derived or isolated from a human gene a selected from the group consisting of: SOD1, TARDBP, FUS, MATR3, SOD1, C9ORF72 (Amyotrophic lateral sclerosis); MAPT, PGRN (Frontotemporal dementia with parkinsonism); CDH23, MYO7A, USH2A (Usher's syndrome); GALC (Krabbe disease); SMPD1, NPC1, NPC2 (Niemann Pick disease); PRNP (prion disease); SCN1A (Dravet syndrome); PINK1, ATPGAP2 (early-onset Parkinson's disease); ATXN1, ATXN2, ATXN3, PLEKHG4, SPTBN2, CACNA1A, ATXN7, TTBK2, PPP2R2B, KCNC3, PRKCG, ITRP1, TBP, KCND1, FGF14 (spinocerebellar ataxias); SCN1A, SCN2A, CACNA1A, GRIN2B, GRIN2A, MECP2, FOXG1, SLC6A1, PRRT2, PTEN, KCNQ2, KCNQ3, STARD7, CLRN1 (genetic epilepsy disorders); ATM (Ataxia-telangiectasia); GLB1 (GM1 gangliosidosis); GBA (Gaucher disease); GM2A (GM2 gangliosidosis); UBE3A (Angelman syndrome); SLC2A1 (glucose transporter deficiency type 1); GLA (Fabry disease); MYOC, OPTN, TBK1, WDR36, CYPIB1 (Open Angle Glaucoma); IDUA (Hurler syndrome or Mucopolysaccharidosis 1); IDS (Hunter syndrome or Mucopolysaccharidosis 2); and CLN3 (Batten disease).

Embodiment 5: The composition of any one of embodiments 2-4, wherein the antisense domain is complementary to sequences derived or isolated from a human gene selected from the group consisting of: OD1, TARDBP, FUS, MATR3, SOD1, C9ORF72 (Amyotrophic lateral sclerosis); MAPT, PGRN (Frontotemporal dementia with parkinsonism); CDH23, MYO7A, USH2A (Usher's syndrome); GALC (Krabbe disease); SMPD1, NPC1, NPC2 (Niemann Pick disease); PRNP (prion disease); SCN1A (Dravet syndrome); PINK1, ATPGAP2 (early-onset Parkinson's disease); ATXN1, ATXN2, ATXN3, PLEKHG4, SPTBN2, CACNA1A, ATXN7, TTBK2, PPP2R2B, KCNC3, PRKCG, ITRP1, TBP, KCND1, FGF14 (spinocerebellar ataxics); SCN1A, SCN2A, CACNA1A, GRIN2B, GRIN2A, MECP2, FOXG1, SLC6A1, PRRT2, PTEN, KCNQ2, KCNQ3, STARD7, CLRN1 (genetic epilepsy disorders); ATM (Ataxia-telangiectasia); GLB1 (GM1 gangliosidosis); GBA (Gaucher disease); GM2A (GM2 gangliosidosis); UBE3A (Angelman syndrome); SLC2A1 (glucose transporter deficiency type 1); GLA (Fabry disease); MYOC, OPTN, TBK1, WDR36, CYPIB1 (Open Angle Glaucoma); IDUA (Hurler syndrome or Mucopolysaccharidosis 1); IDS (Hunter syndrome or Mucopolysaccharidosis 2); and CLN3 (Batten disease).

Embodiment 6: The composition of embodiment 1, wherein the intronic domains promote trans-splicing primarily in liver tissue and are derived or isolated from genes selected from the group consisting of: ORM1, ORM2, PLG, ALDOB, CYP2C8, CPS1, ADH4, PON1, CFHR3, C6, DBI, ITIH3, ITIH4, C4BPB, PTGR1, C5, LBP, PRDX1, HPD, UBB, HPR, ADH6, RPL15, CXCL2, DPYS, HAL, SLC39A14, AQP9, SERPINA10, ADH1A, HABP2, SERPINF2, ECI2, APOM, KLKB1, ACSM5, SLCO1B3, ASGR2, IL1RAP, PSMA1, GRB14, LRRC75A-AS1, BCHE, EPHX1, DMGDH, MST1, ACSM3, SEPT2, TFR2, NDUFC1, NR1H4, AFMID, HNMT, TTC39C, RDH16, GPAM, SLC13A5, SERF2, PICALM, PCSK6, TOM1L1, ANPEP, EBNA1BP2, RCL1, DEPDC7, BTG1, KRT8, ABCA6, GJB1, KMO, HSD17B13, LEAP2, PECR, LEPR, CTNND1, ENSA, MASP2, KYNU, ABCB4, DNASE1L3, ACSL5, UBE2V1, SPTBN1, RBPMS, RAB13, IL1R1, SERPINA5, FGGY, TMEM220, ACSS2, VNN1, CTAGE5, MS4A6A, MFSD2A, SKAP1, VDAC2, SLC22A7, PPP2R5C, SORBS2, CDV3, CSAD, TRPM8, TOMM5, SLCO2B1, MICU1, NCOR1, GSAP, CBWD5, ENPP1, SPATS2L, FKBP5, NR3C1, IGF2, IGF1, PLSCR4, MYLK, SLC27A5, ALDH5A1, TXLNGY, PSMB5, MOCOS, PET100, SGK1, AZI2, TJP2, FMO4, STEAP1, DMD, RNF14, RBM34, PABPC1L, ZCCHC9, ANO6, AGMAT, MASP1, IL17RB, MRPS27, MLIP, MON2, ETF1, SULT1B1, GBP7, PRKACA, PI4K2B, SP100, TMED6, NFU1, RMDN2, IP6K2, CCNB1IP1, PCBP1-AS1, SLC39A1, and PHF11

Embodiment 7: The composition of embodiment 6, wherein the intronic domains promote trans-splicing primarily in liver tissue and are selected from the group consisting of SEQ ID NO 493-SEQ ID NO 798.

Embodiment 8: The composition of any one of embodiments 6-7, wherein the replacement domain is derived or isolated from a human gene a selected from the group consisting of: GLB1 (GM1 gangliosidosis); GBA (Gaucher disease); GM2A (GM2 gangliosidosis); PCSK9, LDLR, APOB, APOE (Familial hypercholesterolemia); GAA (Pompe disease); MYOC, OPTN, TBK1, WDR36, CYPIB1 (Open Angle Glaucoma); IDS (Hunter syndrome or Mucopolysaccharidosis 2); IDUA (Hurler syndrome or Mucopolysaccharidosis 1); CLN3 (Batten disease); F9 (Hemophilia B); and F8 (Hemophilia A).

Embodiment 9: The composition of any one of embodiments 6-8, wherein the antisense domain is complementary to sequences derived or isolated from a human gene selected from the group consisting of: GLB1 (GM1 gangliosidosis); GBA (Gaucher disease); GM2A (GM2 gangliosidosis); PCSK9, LDLR, APOB, APOE (Familial hypercholesterolemia); GAA (Pompe disease); MYOC, OPTN, TBK1, WDR36, CYPIB1 (Open Angle Glaucoma); IDS (Hunter syndrome or Mucopolysaccharidosis 2); IDUA (Hurler syndrome or Mucopolysaccharidosis 1); CLN3 (Batten disease); F9 (Hemophilia B); and F8 (Hemophilia A).

Embodiment 10: The composition of embodiment 1, wherein the intronic domains promote trans-splicing primarily in heart tissue and are derived or isolated from genes selected from the group consisting of: TNNI3, TNNT2, MB, FHL2, CSRP3, FHL1, UBB, MYOM2, CD36, GSN, PDHA1, CRIP2, POPDC2, SORBS2, CPT1B, RYR2, ANXA11, ITGA7, SORBS1, NDUFC1, NEXN, MYH7B, ABLIM1, NEBL, CAST, NME2, CYB5R3, TRDN, TACC2, NDUFA5, MYL12B, FHOD3, CALCOCO2, NCAM1, GPD1L, SGCA, AQP1, AKAP1, ARL6IP4, LRRC39, TTN, TSC22D1, PPP1R12B, OBSL1, MYO15B, PKIG, HMOX2, CDK18, BTNL9, ITGB1BP2, SEPT2, CAV2, DTNA, C15orf41, ATP1A1, BCO2, MYL4, ADCY5, CLIC5, SLC8A1, RRAD, AKAP13, AK3, MYO1C, FGF12, LAMA5, ADAM15, WNK2, SUN1, KLHL7, RNF207, HIF3A, HSPG2, LPCAT4, ADH1B, ENY2, RGS3, FRMD3, CCNB1IP1, PHC2, STAB1, HAND2-AS1, PTPN3, PARVB, PKD1, SPEG, SYNE2, ZNF638, SEPT4, DAG1, SLC12A7, FBXO18, FAM107A, RBM17, MYPN, GRIP2, SCN5A, WFS1, RABGAP1L, SYNE1, HLA-DRB5, SNRPN, MEG3, RAPGEF3, SEC13, C1QTNF1, UCKL1, FRY, IP6K2, CUX1, ANK3, MYH14, ADCY4, C3orf18, MYLK3, PXN, TSPAN18, PDE1C, C6orf48, BCS1L, ATG4D, HRAS, TENM2, PLXNB1, N4BP2L2, TNXB, LTBP1, MAPK8IP3, COL1A1, GOAT, ACOT11, MTUS2, VPS45, and KCNIP2.

Embodiment 11: The composition of embodiment 10, wherein the intronic domains promote trans-splicing primarily in heart tissue and are selected from the group consisting of SEQ ID NO 799-SEQ ID NO 1104.

Embodiment 12: The composition of embodiments 10 or 11, wherein the replacement domain is derived or isolated from a human gene a selected from the group consisting of: LAMP2 (Danon disease); GLA (Fabry disease); SLC2A1 (glucose transporter deficiency type 1); UBE3A (Angelman syndrome); MYOC, OPTN, TBK1, WDR36, CYPIB1 (Open Angle Glaucoma); IDUA (Hurler syndrome or Mucopolysaccharidosis 1); IDS (Hunter syndrome or Mucopolysaccharidosis 2); CLN3 (Batten disease); LMNA (Limb-girdle muscular dystrophy type 1B); DMD (Duchenne muscular dystrophy); DYSF (Limb-girdle muscular dystrophy type 2B); SGCB (Limb-girdle muscular dystrophy type 2E); SGCG (Limb-girdle muscular dystrophy type 2C); SGCA (Limb-girdle muscular dystrophy type 2D); SGCD (Limb-girdle muscular dystrophy type 2F); DUX4, D4Z4 (Facioscapulohumeral muscular dystrophy); F9 (Hemophilia B); F8 (Hemophilia A).

Embodiment 13: The composition of any one of embodiments 10-12, wherein the antisense domain is complementary to sequences derived or isolated from a human gene selected from the group consisting of: LAMP2 (Danon disease); GLA (Fabry disease); SLC2A1 (glucose transporter deficiency type 1); UBE3A (Angelman syndrome); MYOC, OPTN, TBK1, WDR36, CYPIB1 (Open Angle Glaucoma); IDUA (Hurler syndrome or Mucopolysaccharidosis 1); IDS (Hunter syndrome or Mucopolysaccharidosis 2); CLN3 (Batten disease); LMNA (Limb-girdle muscular dystrophy type 1B); DMD (Duchenne muscular dystrophy); DYSF (Limb-girdle muscular dystrophy type 2B); SGCB (Limb-girdle muscular dystrophy type 2E); SGCG (Limb-girdle muscular dystrophy type 2C); SGCA (Limb-girdle muscular dystrophy type 2D); SGCD (Limb-girdle muscular dystrophy type 2F); DUX4, D4Z4 (Facioscapulohumeral muscular dystrophy); F9 (Hemophilia B); F8 (Hemophilia A).

Embodiment 14: The composition of embodiment 1, wherein the intronic domains promote trans-splicing primarily in retinal tissue and are derived or isolated from genes selected from the group consisting of: ADGRV1, AMPH, ATP1B2, BSG, CASK, CC2D2A, CEP290, CLTC, DMD, DOC2B, EFR3A, ERP44, FARP2, FN3KRP, GRAMD1B, GSS, HCN1, IFT140, IMPDH1, INTS13, KCTD5, KIF1B, KIF1B, KMT2D, MADD, MAN2A2, MARCH1, MSI2, NAP1L4, PPP3CC, PROM1, PTPRF, PTPRF, RIMS2, SLC17A7, SLC4A7, STXBP5, TTC8, UNC13B, UNC13B, WDR47, and YKT6.

Embodiment 15: The composition of embodiment 14, wherein the replacement domain is derived or isolated from a human gene a selected from the group consisting of: USHA2A, RPGR, RP2, RHO, PRPF31, USH1F, PRPF3, PRPF6 (Retinitis pigmentosa)

Embodiment 16: The composition of embodiments 14 or 15, wherein the antisense domain is complementary to sequences derived or isolated from a human gene selected from the group consisting of: USHA2A, RPGR, RP2, RHO, PRPF31, USH1F, PRPF3, PRPF6 (Retinitis pigmentosa)

Embodiment 17: The composition of embodiment 1, wherein the intronic domains promote trans-splicing primarily in muscle tissue and are derived or isolated from genes selected from the group consisting of: ALDOA, TNNT3, MYBPC1, ATP2A1, NEB, STAC3, CMYA5, MYH7, TNNI1, CA3, ASB5, PLIN2, TRDN, OBSCN, RYR1, RAP1A, SPARCL1, CSNK1A1, NPEPPS, AKR1C3, IDI2, MBNL1, FNDC5, TMOD4, ATP6V1F, DAP3, KPNB1, NOMO2, BEST3, HP1BP3, SEPT2, TIMM23, MYPN, DHRS7C, PALLD, POLR2J3, KANSL3, THOC7, MEF2C, NOMO1, YWHAZ, ZBTB16, SPAG9, CCZ1, MYLK2, ASAH1, GTF2I, CCPG1, SURF4, SMARCA2, CLTCL1, SLC6A6, CHRNB1, MAFG, COMMD6, SLC7A8, CDK11B, and DNASE1L1

Embodiment 18: The composition of embodiment 17, wherein the intronic domains promote trans-splicing primarily in muscle tissue and are selected from the group consisting of SEQ ID NO 1105-SEQ ID NO 1220.

Embodiment 19: The composition of embodiments 17 or 18, wherein the replacement domain is derived or isolated from a human gene a selected from the group consisting of: LAMP2 (Danon disease); GLA (Fabry disease); SLC2A1 (glucose transporter deficiency type 1); UBE3A (Angelman syndrome); MYOC, OPTN, TBK1, WDR36, CYPIB1 (Open Angle Glaucoma); IDUA (Hurler syndrome or Mucopolysaccharidosis 1); IDS (Hunter syndrome or Mucopolysaccharidosis 2); CLN3 (Batten disease); LMNA (Limb-girdle muscular dystrophy type 1B); DMD (Duchenne muscular dystrophy); DYSF (Limb-girdle muscular dystrophy type 2B); SGCB (Limb-girdle muscular dystrophy type 2E); SGCG (Limb-girdle muscular dystrophy type 2C); SGCA (Limb-girdle muscular dystrophy type 2D); SGCD (Limb-girdle muscular dystrophy type 2F); DUX4, D4Z4 (Facioscapulohumeral muscular dystrophy); F9 (Hemophilia B); and F8 (Hemophilia A)

Embodiment 20: The composition of any one of embodiments 17-19, wherein the antisense domain is complementary to sequences derived or isolated from a human gene selected from the group consisting of: LAMP2 (Danon disease); GLA (Fabry disease); SLC2A1 (glucose transporter deficiency type 1); UBE3A (Angelman syndrome); MYOC, OPTN, TBK1, WDR36, CYPIB1 (Open Angle Glaucoma); IDUA (Hurler syndrome or Mucopolysaccharidosis 1); IDS (Hunter syndrome or Mucopolysaccharidosis 2); CLN3 (Batten disease); LMNA (Limb-girdle muscular dystrophy type 1B); DMD (Duchenne muscular dystrophy); DYSF (Limb-girdle muscular dystrophy type 2B); SGCB (Limb-girdle muscular dystrophy type 2E); SGCG (Limb-girdle muscular dystrophy type 2C); SGCA (Limb-girdle muscular dystrophy type 2D); SGCD (Limb-girdle muscular dystrophy type 2F); DUX4, D4Z4 (Facioscapulohumeral muscular dystrophy); F9 (Hemophilia B); and F8 (Hemophilia A)

Embodiment 21: The composition of embodiment 1, wherein the the intronic domains carry binding sites that are preferentially-targeted by RNA-binding proteins with disease-causing mutations.

Embodiment 22: The composition of any one of embodiments 1-21, wherein the intronic domains carry binding sites for RNA-binding proteins selected from the group consisting of: DDX39, DDX3X, DDX3Y, DDX41, DDX5, DHX16, DHX8, DHX9, INTS6, DDX48, HNRNPA0, HNRNPA1, HNRNPA2B1, HNRPA3, HNRPAB, HNRPC, HNRPDL, HNRPF, HNRPH1, HNRPH2, HNRPLL, HNRPM, HNRPUL1, PTBP1, PTBP2, RBMXL2, SYNCRIP, CLK3, CLK4, SRPK1, SRPK2, SRPK3, ARS2, BUB3, C7ORF30, CIRBP, CRNKL1, CSDA, DNAJC8, EEF1A1, EIF3S10, FMR1, GRSF1, GTL3, IGF2BP3, IK, ILF2, ILF3, MATR3, MBNL1, MBNL2, MBNL3, MOV10, MSI1, MSI2, MYEF2, NHP2L1, PABPC1, PCBP1, PCBP2, PCBP4, PPIE, PPIL2, PPIL2, PPM1G, PPWD1, PRPF6, PSEN1, RALY, RBM15, RBM9, RBMS1, RBMX, RDBP, ROD1, SSB, THOC3, THOC4, TNPO1, TOP1MT, ZFP207, EFTUD2, LSM1, LSM2, LSM3, LSM4, LSM7, LSM8, NA, SART1, SF3A1, SF3A2, SF3A3, SF3B1, SF3B2, SNRPA, SNRPA1, SNRPB, SNRPC, SNRPD1, SNRPD2, SNRPG, SNRPN, TXNL4, U2AF1L1, U2AF1L2, USP39, WDR57, PRPF31, PRPF4, PRPF40A, RBM17, NA, A2BP1, ACIN1, ASCC3L1, BCAS2, BRUNOL4, BUD13, BUD31, CD2BP2, CDC40, CDC5L, CRKRS, CUGBP2, ELAVL1, ELAVL1, ELAVL1, ELAVL2, ELAVL3, ELAVL4, FUSIP1, HSPA1B, HSPA5, HSPA8, NCBP2, NONO, NOVA1, NUDT21, PLRG1, PRPF19, PSIP1, RBM8A, RBMS1, SMU1, SNW1, TCERG1, TIA1, TIAL1, TNRC4, WBP11, WTAP, XAB2, YBX1, SFRS1, SFRS10, SFRS16, SFRS3, SFRS4, and SFRS6

Embodiment 23: The composition of any one of embodiments 1-22, wherein the trans-splicing RNA comprises an untranslated region that increases that alters the processing of the trans-splicing nucleic acid.

Embodiment 24: the composition of any one of embodiments 1-23, wherein the sequence comprising the trans-splicing nucleic acid comprises a sequence that is bound by an RNA-binding protein that increases the trans-splicing efficiency.

Embodiment 25: the composition of any of one embodiments 1-24, wherein the trans-splicing nucleic acid is RNA, DNA, a DNA/RNA hybrid, nucleic acid analog, a chemically-modified nucleic acid, or a chimera composed of two or more nucleic acids or nucleic acid analogs.

Embodiment 26: the composition of any of one embodiments 1-25, wherein the wherein the trans-splicing nucleic acid molecule further comprises a heterologous promoter.

Embodiment 27: the composition of any of one embodiments 1-26, wherein the promoter is isolated or derived from a promoter capable of driving expression of a transfer RNA (tRNA).

### EXAMPLES

### Example 1: Methods for Identification of Intronic Domains that Promote Trans-Splicing in Muscle

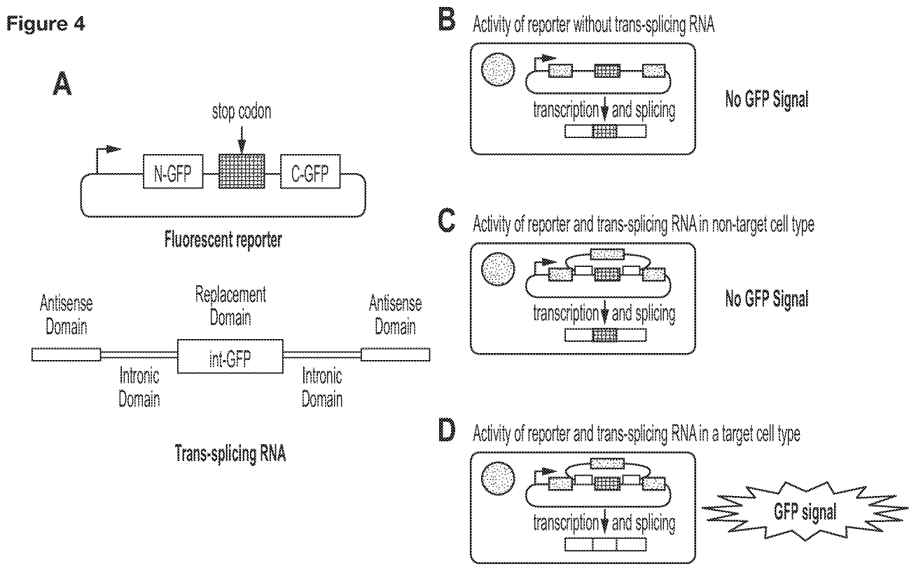

In order to identify Intronic Domains that will promote trans-splicing in muscle but not other tissues, the following experimental strategy is conducted: 1) analysis of unbiased RNA sequencing data from muscle, brain, liver, and kidney, and 2) identification of exons that are included primarily in muscle (“muscle-specific exons”), then 3) extraction of intronic sequences that are adjacent to the muscle-specific exons (“muscle-specific introns”). These muscle-specific introns promote inclusion of adjacent exons during RNA splicing, but RNA trans-splicing is less-efficient and most intronic sequences do not function efficiently in the context of trans-splicing. In order to identify muscle-specific introns that function in the context of RNA trans-splicing (“muscle-specific trans-splicing introns” or MSTI's), candidate MSTI's are selected from the group of muscle-specific introns and compared in a trans-splicing tissue culture experiment. The readout for the trans-splicing tissue culture experiment relies on a reporter that carries a GFP coding sequence interrupted by an exon carrying a stop codon and flanked by two introns (FIG. 4A). When transfected into a mammalian cell line, this reporter does not produce active GFP protein (FIG. 4B). The candidate MSTI's are placed in a trans-splicing RNA vector carrying antisense sequences that target the reporter and an exon that contains no stop codon (FIG. 4A). Successful trans-splicing among the trans-splicing vector and the reporter results in production of active GFP protein (FIG. 4B). In this manner, GFP expression serves as a proxy for the efficiency associated with RNA trans-splicing afforded by the MSTI. GFP expression among experiments conducted in muscle cell lines compared to brain, liver, and kidney cell lines or primary cells is assessed. MSTI's will be identified based on the following criteria: both 1) high GFP expression in muscle cell lines or primary cells, and 2) low GFP expression in brain, liver, and kidney cell lines or primary cells. These MSTI's serve as Intronic Domains that promote tissue-specific trans-splicing in muscle but not other tissues.

### Example 2: Identification of Intronic Domains that Promote Trans-Splicing in Brain, Liver, or Kidney Tissue

In order to identify Intronic Domains that will promote trans-splicing in other tissues such as brain, liver, or kidney, a similar experiment as above is conducted. Here, introns that promote tissue-specific exon inclusion in the respective tissue will be compared in a similar assay. The candidate tissue-specific intronic sequences are similarly placed in a trans-splicing RNA vector and successful tissue-specific intronic sequences are identified based upon the following criteria: both 1) high GFP expression in cells derived from the chosen cell lines or primary cells, and 2) low GFP expression in cell lines or primary cells derived from other tissues. Intronic sequences that fit these criteria will serve as Intronic Domains that promote tissue-specific trans-splicing in a chosen tissue but not other tissues.

### Example 3: Identification of a Therapeutic Trans-Splicing System to Treat Limb-Girdle Muscular Dystrophy (LGMD) that is Active Exclusively in Muscle Tissue

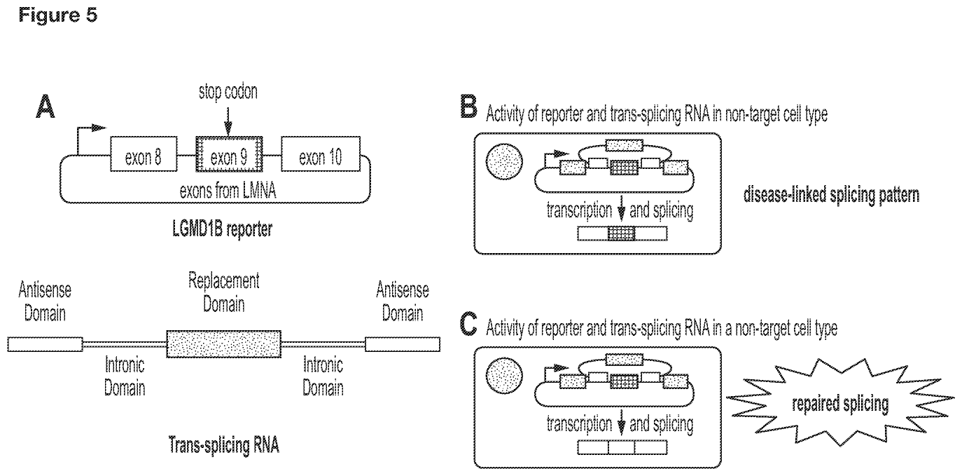

LGMD1B is a devastating muscle disease that causes progressive muscle wasting and death. The disease is caused by mutations in the gene LMNA that cause dysfunctional behavior of the gene (specifically, inappropriate inclusion of exon 9 which carries a stop codon). In order to identify trans-splicing systems that address this dysfunctional inclusion of exon 9 in LMNA in order to treat LGMD1B, we compare tens of systems that replace exon 9 with a short exon that carries no stop codons. We constructs 50+ trans-splicing RNA constructs that carry introns that are effectively spliced in muscle but not other tissues (SEQ ID NO 1105-SEQ ID NO 1220) and carry a replacement a reporter that carries a replacement for exon 9 from LMNA gene (FIG. 5A). Next, we Then, we co-transfect this reporter and individual trans-splicing RNA constructs in human cells lines derived from muscle or another non-muscle cell-type such as HEK cells. Next, we conduct RT-PCR measurements in both cell types primers flanking exon 9 to identify degree of replacement of exon 9 by the trans-splicing RNA replacement sequence. Finally, high-quality trans-splicing RNA systems are identified by the following metric: 1) high levels of replacement sequence inclusion in the LMNA reporter in muscle cells (>50%), with 2) low levels of replacement sequence inclusion in the LMNA reporter in HEK cells (<20%).

### Cells and Tissues

In some embodiments of the compositions and methods of the disclosure, a cell of the disclosure is a eukaryotic cell. In some embodiments, the cell is a mammalian cell. In some embodiments, the cell is a bovine, murine, feline, equine, porcine, canine, simian, or human cell. In some embodiments, the cell is a non-human mammalian cell such as a non-human primate cell. In some embodiments, a cell of the disclosure is a somatic cell. In some embodiments, a cell of the disclosure is a germline cell. In some embodiments, a germline cell of the disclosure is not a human cell.

In some embodiments of the compositions and methods of the disclosure, a cell of the disclosure is a stem cell. In some embodiments, a cell of the disclosure is an embryonic stem cell. In some embodiments, an embryonic stem cell of the disclosure is not a human cell. In some embodiments, a cell of the disclosure is a multipotent stem cell or a pluripotent stem cell. In some embodiments, a cell of the disclosure is an adult stem cell. In some embodiments, a cell of the disclosure is an induced pluripotent stem cell (iPSC). In some embodiments, a cell of the disclosure is a hematopoietic stem cell (HSC).

In some embodiments of the compositions and methods of the disclosure, an immune cell of the disclosure is a lymphocyte. In some embodiments, an immune cell of the disclosure is a T lymphocyte (also referred to herein as a T-cell). Exemplary T-cells of the disclosure include, but are not limited to, naïve T cells, effector T cells, helper T cells, memory T cells, regulatory T cells (Tregs) and Gamma delta T cells. In some embodiments, an immune cell of the disclosure is a B lymphocyte. In some embodiments, an immune cell of the disclosure is a natural killer cell. In some embodiments, an immune cell of the disclosure is an antigen-presenting cell.

In some embodiments of the compositions and methods of the disclosure, a muscle cell of the disclosure is a myoblast or a myocyte. In some embodiments, a muscle cell of the disclosure is a cardiac muscle cell, skeletal muscle cell or smooth muscle cell. In some embodiments, a muscle cell of the disclosure is a striated cell.

In some embodiments of the compositions and methods of the disclosure, a somatic cell of the disclosure is an epithelial cell. In some embodiments, an epithelial cell of the disclosure forms a squamous cell epithelium, a cuboidal cell epithelium, a columnar cell epithelium, a stratified cell epithelium, a pseudostratified columnar cell epithelium or a transitional cell epithelium. In some embodiments, an epithelial cell of the disclosure forms a gland including, but not limited to, a pineal gland, a thymus gland, a pituitary gland, a thyroid gland, an adrenal gland, an apocrine gland, a holocrine gland, a merocrine gland, a serous gland, a mucous gland and a sebaceous gland. In some embodiments, an epithelial cell of the disclosure contacts an outer surface of an organ including, but not limited to, a lung, a spleen, a stomach, a pancreas, a bladder, an intestine, a kidney, a gallbladder, a liver, a larynx or a pharynx. In some embodiments, an epithelial cell of the disclosure contacts an outer surface of a blood vessel or a vein.

In some embodiments of the compositions and methods of the disclosure, a brain cell of the disclosure is a neuronal cell. In some embodiments, a neuron cell of the disclosure is a neuron of the central nervous system. In some embodiments, a neuron cell of the disclosure is a neuron of the brain or the spinal cord. In some embodiments, a neuron cell of the disclosure is a neuron of a cranial nerve or an optic nerve. In some embodiments, a neuron cell of the disclosure is a neuron of the peripheral nervous system. In some embodiments, a neuron cell of the disclosure is a neuroglial or a glial cell. In some embodiments, a glial of the disclosure is a glial cell of the central nervous system including, but not limited to, oligodendrocytes, astrocytes, ependymal cells, and microglia. In some embodiments, a glial of the disclosure is a glial cell of the peripheral nervous system including, but not limited to, Schwann cells and satellite cells.

In some embodiments of the compositions and methods of the disclosure, a liver cell of the disclosure is a hepatocytes. In some embodiments, a liver cell of the disclosure is a hepatic stellate cell. In some embodiments, a liver cell of the disclosure is Kupffer cell. In some embodiments, a liver cell of the disclosure is a sinusoidal endothelial cells.

In some embodiments of the compositions and methods of the disclosure, a retinal cell of the disclosure is a photoreceptor. In some embodiments, a photoreceptor cell of the disclosure is a rod. In some embodiments, a retinal cell of the disclosure is cone. In some embodiments, a retinal cell of the disclosure is a bipolar cell. In some embodiments, a retinal cell of the disclosure is a ganglion cell. In some embodiments, a retinal cell of the disclosure is a horizontal cell. In some embodiments, a retinal cell of the disclosure is an amacrine cell.

In some embodiments of the compositions and methods of the disclosure, a heart cell of the disclosure is a cardiomyocyte. In some embodiments, a heart cell of the disclosure is a cardiac pacemaker cell.

In some embodiments of the compositions and methods of the disclosure, a somatic cell of the disclosure is a primary cell.

In some embodiments of the compositions and methods of the disclosure, a somatic cell of the disclosure is a cultured cell.

In some embodiments of the compositions and methods of the disclosure, a somatic cell of the disclosure is in vivo, in vitro, ex vivo or in situ.

In some embodiments of the compositions and methods of the disclosure, a somatic cell of the disclosure is autologous or allogeneic.

### INCORPORATION BY REFERENCE

Every document cited herein, including any cross referenced or related patent or application is hereby incorporated herein by reference in its entirety unless expressly excluded or otherwise limited. The citation of any document is not an admission that it is prior art with respect to any invention disclosed or embodimented herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

### OTHER EMBODIMENTS

While particular embodiments of the disclosure have been illustrated and described, various other changes and modifications can be made without departing from the spirit and scope of the disclosure. The scope of the appended claims includes all such changes and modifications that are within the scope of this disclosure.

1.-31. (canceled)

32. A trans-splicing nucleic acid, comprising: (a) one or more replacement domains that encode a therapeutic sequence; (b) one or more intronic domains, wherein the one or more intronic domains promote ribonucleic acid (RNA) splicing of the replacement domain primarily in a specific tissue or cell type, and wherein the one or more intronic domains comprise one or more RNA-binding protein sites configured to promote tissue-specific splicing patterns; and (c) one or more antisense domains that promote binding to a target RNA molecule.

33. The trans-splicing nucleic acid of claim 32, wherein the intronic domains comprise one or more sequences that are preferentially targeted by RNA-binding proteins carrying disease-associated mutations.

34. The trans-splicing nucleic acid of claim 32, further comprising a 3′ untranslated region that improves a trans-splicing efficiency of the trans-splicing nucleic acid to the target RNA.

35. The trans-splicing nucleic acid of claim 32, further comprising a 5′ untranslated region that improves the trans-splicing efficiency of the trans-splicing nucleic acid to the target RNA.

36. The trans-splicing nucleic acid of claim 32, further comprising an RNA-binding protein that strengthens the interaction among the trans-splicing nucleic acid molecule and the target RNA molecule, thereby increasing a trans-splicing efficiency of the trans-splicing nucleic acid to the target RNA.

37. The trans-splicing nucleic acid of claim 32, wherein the trans-splicing nucleic acid comprises RNA, DNA, a DNA/RNA hybrid, a nucleic acid analog, a chemically-modified nucleic acid, or a chimera composed of two or more nucleic acids or nucleic acid analogs.

38. A method for modifying a target RNA molecule in a cell, the method comprising administering a trans-splicing nucleic acid to the cell, wherein the trans-splicing nucleic acid comprises (a) one or more replacement domains that encode a therapeutic sequence; (b) one or more intronic domains, wherein the one or more intronic domains promote ribonucleic acid (RNA) splicing of the replacement domain primarily in a specific tissue or cell type, and wherein the one or more intronic domains comprise one or more RNA-binding protein sites configured to promote tissue-specific splicing patterns; and (c) one or more antisense domains that promote binding to the target RNA molecule.

39. The method of claim 38, wherein the one or more intronic domains comprise one or more sequences that are preferentially targeted by RNA-binding proteins carrying disease-associated mutations.

40. The method of claim 38, wherein the trans-splicing nucleic acid further comprises a 3′ untranslated region that improves the trans-splicing efficiency of the trans-splicing nucleic acid to the target RNA.

41. The method of claim 38, wherein the trans-splicing nucleic acid further comprises a 5′ untranslated region that improves the trans-splicing efficiency of the trans-splicing nucleic acid to the target RNA.

42. The method of claim 38, further comprising providing an RNA-binding protein that strengthens the interaction among the trans-splicing nucleic acid and the target RNA molecule, and wherein a strengthening of the interaction among the trans-splicing nucleic acid molecule and the target RNA molecule increases a trans-splicing efficiency of the trans-splicing nucleic acid to the target RNA.

43. The method of claim 38, wherein the trans-splicing nucleic acid comprises RNA, DNA, a DNA/RNA hybrid, a nucleic acid analog, a chemically-modified nucleic acid, or a chimera composed of two or more nucleic acids or nucleic acid analogs.

44. The method of claim 38, further comprising inserting the therapeutic sequence into the target RNA.

45. The method of claim 44, wherein the therapeutic sequence is not inserted into a target RNA in a second tissue or a second cell type.

46. A trans-splicing nucleic acid, comprising: (a) one or more replacement domains that encode a therapeutic sequence; (b) one or more intronic domains configured to promote ribonucleic acid (RNA) splicing of the one or more replacement domains primarily to a target RNA in a specific tissue or a specific cell type, and wherein the one or more intronic domains comprise a sequence configured to be bound by an RNA-binding protein that increases a trans-splicing efficiency of the therapeutic sequence to the target RNA; and (c) one or more antisense domains that promote binding to a target RNA molecule.

47. The trans-splicing nucleic acid of claim 46, wherein the one or more intronic domains comprise a sequence preferentially targeted by an RNA-binding protein carrying one or more disease-associated mutations.

48. The trans-splicing nucleic acid of claim 46, further comprising a 3′ untranslated region that improves the trans-splicing efficiency of the therapeutic sequence to the target RNA.

49. The trans-splicing nucleic acid of claim 46, further comprising a 5′ untranslated region that improves the trans-splicing efficiency of the therapeutic sequence to the target RNA.

50. The trans-splicing nucleic acid of claim 46, further comprising the RNA-binding protein that increases the trans-splicing efficiency of the therapeutic sequence to the target RNA.

51. The trans-splicing nucleic acid of claim 46, wherein the trans-splicing nucleic acid comprises RNA, DNA, a DNA/RNA hybrid, a nucleic acid analog, a chemically-modified nucleic acid, or a chimera composed of two or more nucleic acids or nucleic acid analogs.
